SEQUENCE LISTING (26 sequences)

SEQ ID NO: 1 - DNA, 34 bp, synthetic construct
  source 1..34: mol_type = other DNA
  misc_feature 1..34: note = Synthetic
gaacattaacctgcgctccagcattgaagataac

SEQ ID NO: 2 - DNA, 13 bp, synthetic construct
  source 1..13: mol_type = other DNA
  misc_feature 1..13: note = Synthetic
cagataagtgcag

SEQ ID NO: 3 - DNA, 200 bp, synthetic construct
  misc_feature 1..200: note = Synthetic
  source 1..200: mol_type = other DNA
ttcaaggtccaatctctgctcaatgattaacctcggtacaagaggtgtactttgtaccctggacgatcaactgccgacgctgtactggttaataattaaccagggttgacggggtagcccgcatagaatatgccccaagcactaccctaagatacacctgtacaatctcatccctatcccaacgccgcatcagcgatgcg

SEQ ID NO: 4 - DNA, 199 bp, synthetic construct
  misc_feature 1..199: note = Synthetic
  source 1..199: mol_type = other DNA
tgtcaatagtactacacctgatcaaaggtcaatagcggtacttacgcaacggcagtgattgctgtgattcaacatccagacaggtggtcctaccttgaccttctatcaatagtaggtgataggctatggcaacagagcatccctctttgatggggtgagtaatggtccaaaggtcggtaccagggcaaacatagactgt

SEQ ID NO: 5 - DNA, 200 bp, synthetic construct
  misc_feature 1..200: note = Synthetic
  source 1..200: mol_type = other DNA
tgcggtgcacctgtaccatttggcaacaatagccctttgatgcagtgattcattgtggtgacacaaggtgataagggtcaaaggtgcactgatttatgactcaatgacccactcttgctagacccactagtcaaatagtcaatgacagcattgagtggaatgttaccctttgatcttctttgacggcacctgtactttgg

SEQ ID NO: 6 - DNA, 200 bp, synthetic construct
  misc_feature 1..200: note = Synthetic
  source 1..200: mol_type = other DNA
gctgtcgcctggcaggtaactattaggacggccccgacagtttgcccattcttctcagtacgcattctacttgagtgtttactaaggtctgtgcaggggcaaaggttcaatgggcccctttcagagggattactcagcaggttgcctaacctgagtgagtcttatgttaatggttaaccgtggtatgaaccaagatcaat

SEQ ID NO: 7 - DNA, 200 bp, synthetic construct
  source 1..200: mol_type = other DNA
  misc_feature 1..200: note = Synthetic
tctcgtcatctaatcgcggaggaatctcagcctcttaacgaagaaatctcagcatcctcggggatttgacgcatcctcggaggatgcgtcctgaggaacttccttgacgaaaacggaagacgtcttcgaaggatgcgccccggatttccaggaatttagagtaatgcaggcgtttgttcaatggtcgattaaaactaaat

SEQ ID NO: 8 - DNA, 198 bp, synthetic construct
  misc_feature 1..198: note = Synthetic
  source 1..198: mol_type = other DNA
tcgcatcacggaagaagatgccttaaggacttccagtagcatccttatcccggaacaatgaagatgcatcacttgagctacgcatttaatacgatgggtggagccgtaatatggctcctttaaggatgtattgatgccttagattacgtcttcaagtatgacgtgatgctaataaatcacagcctacgcattggtacc

SEQ ID NO: 9 - DNA, 201 bp, synthetic construct
  source 1..201: mol_type = other DNA
  misc_feature 1..201: note = Synthetic
acggcgaggccgatggcacgtgacggatgccgacttcgtcagtctgatggcatcgaggaatcatattgtattccgcctggcatcctccagtcgtggcacggttcaggatacatacgtatctccgggatgcgtcgcaagacgtgcctggcgtctatacgtcacctccttacgccttggcatctttctccatccttggaaaat

SEQ ID NO: 10 - protein, 401 aa, synthetic construct
  REGION 1..401: note = IscB Protein
MSTDATLIRTTPSHAEADATDTLVATPLMPPRRVISPWPGPGEGQSLMRIPVVDIRGMALMPCTPAKARHLLKSGNARPKRNKLGLFYVQLSYEQEPDNQSLVAGVDPGSKFEGLSVVGTKDTVLNLMVEAPDHVKGAVQTRRTMRRARRQRKWRRPKRFHNRLNRMQRIPPSTRSRWEAKARIVAHLRTILPFTDVVVEDVQAVTRKGKGGTWNGSFSPVQVGKEHLYRLLRAMGLTLHLREGWQTKELREQHGLKKTKSKSKQSFESHAVDSWVLAASISGAEHPTCTRLWYMVPAILHRRQLHRLQASKGGVRKPYGGTRSLGVKRGTLVEHKKYGRCTVGGVDRKRNTISLHEYRTNTRLTQAAKVETCRVLTWLSWRSWLLRGKRTSSKGKGSHSS

SEQ ID NO: 11 - protein, 432 aa, synthetic construct
  REGION 1..432: note = IscB Protein
MQPAKQQNWVFQINGDKQPLDMINPGRCRELQNRGKLASFRRFPYVVIQQQTIENPQTKEYILKIDPGSQWTGFAIQCGNDILFRAELNHRGEAIKFDLVKRAWFRRGRRSRNLRYRKKRLNRAKPEGWLAPSIRHRVLTVETWIKRFMRYCPIAWIEIEQVRFDTQKLANPEIDGVEYQQGELQGYEVREYLLQKWGRKCAYCGTENVPLEVEHIQSKSKGGSSRIGNLTLACHVCNVKKGNLDVRDFLAKSPDILNQVLENSTKPLKDAAAVNSTRYAIVKMAKSICENVKCSSGARTKMNRVRQGLEKTHSLDAACVGESGASIRVLTDRPLLITCKGHGSRQSIRVNASGFPAVKNAKTVFTHIAAGDVVRFTIGKDRKKAQAGTYTARVKTPTPKGFEVLIDGARISLSTMSNVVFVHRSDGYGYEL

SEQ ID NO: 12 - protein, 461 aa, synthetic construct
  REGION 1..461: note = IscB Protein
MAVFVIDKHKRPLMPCSEKRARLLLERGRAVVHRQVPFVIRLKDRTVQHSAVQPLRVALDPGSRATGMALVREKNTVDTGTGEVYRERIALNLFELVHRGHRIREQLDQRRNFRRRRRGANLRYRAPRFDNRRRPPGWLAPSLQHRVDTTMAWVRRLCRWAPASAIGIETVRFDTQRLQNPEISGVEYQQGALAGCEVREYLLEKWGRKCAYCGAENVPLEIEHIVPKSRGGSDRVSNLALACRACNQAKGNRDVRAFLADQPERLARILAQAKAPLKDAAAVNATRWALYRALVDTGLPVEAGTGGRTKWNRTRLGLPKTHALDALCVGQVDQVRHWRVPVLGIRCAGRGSYRRTRLTRHGFPRGYLTRNKSAFGFQTGDLIRAVVTKGKKAGTYLGRIAIRASGSFNIQTPMGVVQGIHHRFCTLLQRADGYGYFVQPKPTEAALSSPRLKAGVSSAGN

SEQ ID NO: 13 - protein, 439 aa, synthetic construct
  REGION 1..439: note = IscB Protein
MTTNVVFVIDTNQKPLQPCSAAVARKLLLRGKAAMFRRYPAVIILKKEVDSVGKPKIELRIDPGSKYTGFALVDSKDNADFIIWGTELEHRGAAICKELTKRSAIRRSRRNRKTRYRKKRFERRKPEGWLAPSLQHRVDTTLTWVKRICKFVPIMSISVEQVKFDLQKLENSDIQGIEYQQGTLAGYTLREALLEHWGRKCAYCDVENVFLEIEHIYPKSKGGSDKFSNLTLACHKCNINKGNKSIDEFLLSDHKRLEQIKLHQKKTLKDAAAVNATRKKLVTTLQEKTFLNVLVSDGASTKMTRLSSSLAKRHWIDAGCVNTTLIVILKTLQPLQVKCNGHGNKQFVTMDAYGFPRKSYEPKKVRKDWKAGDIIRVTKKDGTMLMGRVKKAAKKLVYIPFGGKEASFSSENAKAIHRSDGYRYSFAAIDSELLQKMAT

SEQ ID NO: 14 - protein, 419 aa, synthetic construct
  REGION 1..419: note = IscB Protein
MPNKYAFVLDSKGKLLDPTKSKKAWYLIRKGKASLVEEYPLIIKLKREVPKDQVNSDKLILGIDDGTKKVGFALVQKCQTKNKVLFKAVMEQRQDVSKKMEERRGYRRYRRSHKRYRPARFDNRSSSKRKGRIPPSILQKKQAILRVVNKLKKYIRIDKIVLEDVSIDIRKLTEGRELYNWEYQESNRLDENLRKATLYRDDCTCQLCGTTETMLHAHHIMPRRDGGADSIYNLITLCKACHKDKVDNNEYQYKDQFLAIIDSKELSDLKSASHVMQGKTWLRDKLSKIAQLEITSGGNTANKRIDYEIEKSHSNDAICTTGLLPVDNIDDIKEYYIKPLRKKSKAKIKELKCFRQRDLVKYTKRNGETYTGYITSLRIKNNKYNSKVCNFSTLKGKIFRGYGFRNLTLLNRPKGLMIV

SEQ ID NO: 15 - protein, 1409 aa, synthetic construct
  REGION 1..1409: note = IscB Protein
MLFNKCIIISINLDFSNKEKCMTKPYSIGLDIGTNSVGWAVITDNYKVPSKKMKVLGNTSKKYIKKNLLGVLLFDSGITAEGRRLKRTARRRYTRRRNRILYLQEIFSTEMATLDDAFFQRLDDSFLVPDDKRDSKYPIFGNLVEEKVYHDEFPTIYHLRKYLADSTKKADLRLVYLALAHMIKYRGHFLIEGEFNSKNNDIQKNFQDFLDTYNAIFESDLSLENSKQLEEIVKDKISKLEKKDRILKLFPGEKNSGIFSEFLKLIVGNQADFRKCFNLDEKASLHFSKESYDEDLETLLGYIGDDYSDVFLKAKKLYDAILLSGFLTVTDNETEAPLSSAMIKRYNEHKEDLALLKEYIRNISLKTYNEVFKDDTKNGYAGYIDGKTNQEDFYVYLKNLLAEFEGADYFLEKIDREDFLRKQRTFDNGSIPYQIHLQEMRAILDKQAKFYPFLAKNKERIEKILTFRIPYYVGPLARGNSDFAWSIRKRNEKITPWNFEDVIDKESSAEAFINRMTSFDLYLPEEKVLPKHSLLYETFNVYNELTKVRFIAESMRDYQFLDSKQKKDIVRLYFKDKRKVTDKDIIEYLHAIYGYDGIELKGIEKQFNSSLSTYHDLLNIINDKEFLDDSSNEAIIEEIIHTLTIFEDREMIKQRLSKFENIFDKSVLKKLSRRHYTGWGKLSAKLINGIRDEKSGNTILDYLIDDGISNRNFMQLIHDDALSFKKKIQKAQIIGDEDKGNIKEVVKSLPGSPAIKKGILQSIKIVDELVKVMGGRKPESIVVEMARENQYTNQGKSNSQQRLKRLEKSLKELGSKILKENIPAKLSKIDNNALQNDRLYLYYLQNGKDMYTGDDLDIDRLSNYDIDHIIPQAFLKDNSIDNKVLVSSASNRGKSDDFPSLEVVKKRKTFWYQLLKSKLISQRKFDNLTKAERGGLLPEDKAGFIQRQLVETRQITKHVARLLDEKFNNKKDENNRAVRTVKIITLKSTLVSQFRKDFELYKVREINDFHHAHDAYLNAVIASALLKKYPKLEPEFVYGDYPKYNSFRERKSATEKVYFYSNIMNIFKKSISLADGRVIERPLIEVNEETGESVWNKESDLATVRRVLSYPQVNVVKKVEEQNHGLDRGKPKGLFNANLSSKPKPNSNENLVGAKEYLDPKKYGGYAGISNSFAVLVKGTIEKGAKKKITNVLEFQGISILDRINYRKDKLNFLLEKGYKDIELIIELPKYSLFELSDGSRRMLASILSTNNKRGEIHKGNQIFLSQKFVKLLYHAKRISNTINENHRKYVENHKKEFEELFYYILEFNENYVGAKKNGKLLNSAFQSWQNHSIDELCSSFIGPTGSERKGLFELTSRGSAADFEFLGVKIPRYRDYTPSSLLKDATLIHQSVTGLYETRIDLAKLGEG

SEQ ID NO: 16 - protein, 1053 aa, synthetic construct
  REGION 1..1053: note = IscB Protein
MKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 17 - protein, 1365 aa, synthetic construct
  REGION 1..1365: note = IscB Protein
KYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 18 - protein, 288 aa, synthetic construct
  REGION 1..288: note = Synthetic
MDPIRSRTPSPARELLSGPQPDGVQPTADRGVSPPAGGPLDGLPARRTMSRTRLPSPPAPSPAFSADSFSDLLRQFDPSLFNTSLFDSLPPFGAHHTEAATGEWDEVQSGLRAADAPPPTMRVAVTAARPPRAKPAPRRRAAQPSDASPAAQVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLN

SEQ ID NO: 19 - protein, 183 aa, synthetic construct
  REGION 1..183: note = Synthetic
RPALESIVAQLSRPDPALAALTNDHLVALACLGGRPALDAVKKGLPHAPALIKRTNRRIPERTSHRVADHAQVVRVLGFFQCHSHPAQAFDDAMTQFGMSRHGLLQLFRRVGVTELEARSGTLPPASQRWDRILQASGMKRAKPSPTSTQTPDQASLHAFADSLERDLDAPSPMHEGDQTRAS

SEQ ID NO: 20 - protein, 10 aa, synthetic construct
  VARIANT 6..7: note = X can be any naturally occurring amino acid
  REGION 1..10: note = Synthetic
  VARIANT 2..4: note = X can be any naturally occurring amino acid
  VARIANT 9: note = X can be any naturally occurring amino acid
LXXXLXXLXL

SEQ ID NO: 21 - protein, 4 aa, synthetic construct
  REGION 1..4: note = Synthetic
KDEL

SEQ ID NO: 22 - protein, 15 aa, synthetic construct
  REGION 1..15: note = Synthetic
GGGGGGGGGGGGGGG

SEQ ID NO: 23 - protein, 15 aa, synthetic construct
  REGION 1..15: note = Synthetic
GGGGSGGGGSGGGGS

SEQ ID NO: 24 - protein, 5 aa, synthetic construct
  REGION 1..5: note = Synthetic
TEFCA

SEQ ID NO: 25 - DNA, 10 bp, synthetic construct
  misc_feature 1..10: note = Synthetic
  source 1..10: mol_type = other DNA
ccccctcccc

SEQ ID NO: 26 - DNA, 15 bp, synthetic construct
  source 1..15: mol_type = other DNA
  misc_feature 1..15: note = Synthetic
  variation 1: note = N is G or C
ncggaagtgacgtca

(19) United States
(12) Patent Application Publication
(10) Pub. No.: US 2026/0055408 A1
(43) Pub. Date: Feb. 26, 2026

(54) CELL-SPECIFIC CIS-REGULATORY ELEMENTS, USES THEREOF, AND METHODS OF GENERATING THE SAME

(71) Applicants: The Broad Institute, Inc., Cambridge, MA (US); President and Fellows of Harvard College, Cambridge, MA (US); Jackson Labs, Bar Harbor, ME (US); Yale University, New Haven, CT (US)

(72) Inventors: Pardis Sabeti, Cambridge, MA (US); Rodrigo Castro, Bar Harbor, ME (US); Ryan Tewhey, Bar Harbor, ME (US); Sagar Gosai, Cambridge, MA (US); Steven Reilly, New Haven, CT (US)

(21) Appl. No.: 19/316,097

(22) Filed: Sep. 2, 2025

Related U.S. Application Data
(63) Continuation of application No. PCT/US2024/018183, filed on Mar. 1, 2024.
(60) Provisional application No. 63/449,531, filed on Mar. 2, 2023.

(51) Int. Cl. C12N 15/113; C12Q 1/6897; G16B 40/20; G16B 40/30

(52) U.S. Cl. CPC C12N 15/113; C12Q 1/6897; G16B 40/20; G16B 40/30

(57) ABSTRACT

Described in certain embodiments herein are computer implemented methods, systems, and computer program products that can be used to identify or engineered cell specific cis-regulatory elements (CREs). Also described herein are cell specific CREs and uses thereof.

### CROSS-REFERENCE TO RELATED APPLICATIONS

This application is a continuation application of PCT/US2024/018183, filed Mar. 1, 2024, which claims the benefit of and priority to U.S. Provisional Patent Application No. 63/449,531, filed on Mar. 2, 2023, the contents of which are incorporated by reference herein in its entirety.

### STATEMENT REGARDING FEDERALLY SPONSORED RESEARCH

This invention was made with government support under Grant Nos. HG009435, HG011329, and HG010669 awarded by the National Institutes of Health. The government has certain rights in the invention.

### SEQUENCE LISTING

This application contains a sequence listing filed in electronic form as an XML file entitled “BROD-5815US_ST26.xml”, created on Aug. 26, 2025, and having a size of 41,550 bytes. The content of the sequence listing is incorporated herein in its entirety.

### TECHNICAL FIELD

The subject matter disclosed herein is generally directed to methods and techniques for identifying and generating cis-regulatory elements (CREs), including cell-type specific and tissue specific CREs, and uses of the CREs.

### BACKGROUND

Gene regulation is fundamental to the identity and survival of every cell. While less than 2% of the human genome is dedicated to protein-coding sequence, at least 19% of the genome is associated with open chromatin or transcription factor binding. However, despite their prevalence in the genome, relatively few cis-regulatory elements (CREs) have been directly shown to regulate a target gene. Quantifying the gene-regulatory potential of DNA at nucleotide resolution remains a difficult problem in genomics. Massively parallel reporter assays (MPRAs) directly characterize cis-regulatory function of DNA sequences with the sensitivity required to measure the impacts of genetic variants accurately. However, it remains intractable to test every element in the human genome using MPRAs. As such there exists a pressing need for methods and techniques for harnessing the regulatory protentional of nucleic acid sequences, particularly in cell or tissue or specific manner.

Citation or identification of any document in this application is not an admission that such a document is available as prior art to the present invention.

### SUMMARY

Described in certain example embodiments herein are computer-implemented method to identify or design cis-regulatory elements with cell-type, cell state, tissue type, and/or environment specific activity comprising (a) receiving, by one or more computing devices, one or more nucleic acid sequences; (b) transferring, by one or more computing devices, the one or more nucleic acid sequences to a deployed machine learning network; (c) processing the one or more nucleic acid sequences with the deployed machine learning network, the deployed machine learning network generated and deployed from a training machine learning network trained on CRE-activity from a massively parallel reporter assay (MPRA) data set that provides empirical cell, tissue, or environment specific and non-specific MPRA CRE-activity measurements to the model; (d) generating, by the deployed machine learning network, a prediction of the CRE activity of the one or more nucleic acid sequences; and (e) transmitting, by one or more computing devices, the predicted CRE activity to a user device associated with a user.

In certain example embodiments, the CRE activity is cell type, cell state, tissue type, or environment specific MPRA CRE-activity.

In certain example embodiments, the one or more nucleic acid sequences is a genome or a portion thereof or an epigenome or portion thereof.

In certain example embodiments, the one or more nucleic acid sequence is a DNA sequence generated from a suitable DNA sequence generation algorithm, optionally evolutionary, probabilistic, simulated annealing, or gradient based updates with random momentum (GRUM).

In certain example embodiments, processing further comprises iterative cell, tissue, or environment specific regulatory optimization of the one or more nucleic acid sequence, wherein iterative cell, tissue, or environment specific regulatory optimization comprises sequentially modifying the nucleic acid sequence in each iteration.

In certain example embodiments, processing further comprises passing the prediction to a cell, tissue, or environment specific regulatory optimizing objective function that maximizes cell specific regulatory activity.

In certain example embodiments, the cell specific regulatory optimizing objective function maximizes the predicted expression of a given sequence in one cell type, cell state, tissue type, or environment while reducing expression in all other cell types, cell states, tissue types, or environments.

In certain example embodiments, the method further comprises updating the one or more nucleic acid sequences in each iteration based on the output of the cell, tissue, or environment specific regulatory optimizing objective function.

In certain example embodiments, the objective function prioritizes nucleic acid sequences with cell type, cell state, tissue type, or environment specific promoter activity, enhancer activity, silencer activity, or insulator activity.

In certain example embodiments, the cell type, cell state, tissue type, or environment specific regulatory activity comprises promoter activity, enhancer activity, silencer activity, or insulator activity.

In certain example embodiments, the machine learning network comprises a neural network, Bayesian network, random forest, matrix factorization, hidden Markov model, support vector machine, K-means clustering, K-nearest neighbor, linear classifiers, logistic classifiers, or any combination thereof.

In certain example embodiments, the neural network comprises deep learning, a convolutional neural network, or a recurrent neural network.

In certain example embodiments, the neural network comprises the convolutional neural network.

In certain example embodiments, the cell, tissue, or environment specific CRE-activity MPRA data set is obtained from a suitable database, optionally CREs centered on variants from the UK Biobank and/or GTEx.

In certain example embodiments, the cell type, cell state, tissue type, or environment specific CRE-activity MPRA data set comprises a plurality of pairs of reference and alternate alleles.

In certain example embodiments, the cell, tissue, or environment specific engineered CREs are cell type, cell state, tissue type, or environment specific engineered CREs.

In certain example embodiments, the cell type, cell state, tissue type, or environment specific CRE-activity MPRA data set was generated using vertebrate cells or invertebrate cells.

In certain example embodiments, the cell type, cell state, tissue type, or environment specific CRE-activity MPRA data set was generated using mammalian, avian, reptilian, fish, or amphibian cells.

In certain example embodiments, the cell type, cell state, tissue type, or environment specific CRE-activity MPRA data set was generated using human or non-human primate cells.

In certain example embodiments, the cell type, cell state, tissue type, or environment specific CRE-activity MPRA data set was generated using plant cells.

In certain example embodiments, the one or more nucleic acid sequence is 200 bases or less.

In certain example embodiments, the training machine learning network comprises unsupervised learning, supervised learning, semi-supervised learning, reinforcement learning, transfer learning, incremental learning, curriculum learning, learning to learn, contrastive learning, or any combination thereof.

Described in certain example embodiments herein are systems to identify or design cis-regulatory elements with cell-type, cell state, tissue type, and/or environment specific activity, comprising a storage device; and a processor communicatively coupled to the storage device, wherein the processor executes application code instructions that are stored in the storage device to cause the system to (a) receive, by one or more computing devices, one or more nucleic acid sequences; (b) transfer, by one or more computing devices, the one or more nucleic acid sequences to a deployed machine learning network; (c) process the one or more nucleic acid sequences with the deployed machine learning network, the deployed machine learning network generated and deployed from a training machine learning network trained on CRE-activity from a massively parallel reporter assay (MPRA) data set that provides empirical cell, tissue, or environment specific and non-specific MPRA CRE-activity measurements to the model, (d) generate, by the deployed machine learning network, a prediction of the CRE activity of the one or more nucleic acid sequences; and (e) transmit, by one or more computing devices, the predicted CRE activity to a user device associated with a user.

In certain example embodiments, the CRE activity is cell type, cell state, tissue type, or environment specific MPRA CRE-activity.

In certain example embodiments, the one or more nucleic acid sequences is a genome or a portion thereof or an epigenome or portion thereof.

In certain example embodiments, the one or more nucleic acid sequence is a DNA sequence generated from a suitable DNA sequence generation algorithm, optionally evolutionary, probabilistic, simulated annealing, or gradient based updates with random momentum (GRUM).

In certain example embodiments, processing further comprises iterative cell, tissue, or environment specific regulatory optimization of the one or more nucleic acid sequence, wherein iterative cell, tissue, or environment specific regulatory optimization comprises sequentially modifying the nucleic acid sequence in each iteration.

In certain example embodiments, processing further comprises passing the prediction to a cell, tissue, or environment specific regulatory optimizing objective function that maximizes cell specific regulatory activity.

In certain example embodiments, the cell specific regulatory optimizing objective function maximizes the predicted expression of a given sequence in one cell type, cell state, tissue type, or environment while reducing expression in all other cell types, cell states, tissue types, or environments.

In certain example embodiments, the system further comprises updating the one or more nucleic acid sequences in each iteration based on the output of the cell, tissue, or environment specific regulatory optimizing objective function.

In certain example embodiments, the objective function prioritizes nucleic acid sequences with cell type, cell state, tissue type, or environment specific promoter activity, enhancer activity, silencer activity, or insulator activity.

In certain example embodiments, the cell type, cell state, tissue type, or environment specific regulatory activity comprises promoter activity, enhancer activity, silencer activity, or insulator activity.

In certain example embodiments, the machine learning network comprises a neural network, Bayesian network, random forest, matrix factorization, hidden Markov model, support vector machine, K-means clustering, K-nearest neighbor, linear classifiers, logistic classifiers, or any combination thereof.

In certain example embodiments, the neural network comprises deep learning, a convolutional neural network, or a recurrent neural network.

In certain example embodiments, the neural network comprises the convolutional neural network.

In certain example embodiments, the cell, tissue, or environment specific CRE-activity MPRA data set is obtained from a suitable database, optionally CREs centered on variants from the UK Biobank and/or GTEx.

In certain example embodiments, the cell type, cell state, tissue type, or environment specific CRE-activity MPRA data set comprises a plurality of pairs of reference and alternate alleles.

In certain example embodiments, the cell, tissue, or environment specific engineered CREs are cell type, cell state, tissue type, or environment specific engineered CREs.

In certain example embodiments, the cell type, cell state, tissue type, or environment specific CRE-activity MPRA data set was generated using vertebrate cells or invertebrate cells.

In certain example embodiments, the cell type, cell state, tissue type, or environment specific CRE-activity MPRA data set was generated using mammalian, avian, reptilian, fish, or amphibian cells.

In certain example embodiments, the cell type, cell state, tissue type, or environment specific CRE-activity MPRA data set was generated using human or non-human primate cells.

In certain example embodiments, the cell type, cell state, tissue type, or environment specific CRE-activity MPRA data set was generated using plant cells.

In certain example embodiments, the one or more nucleic acid sequence is 200 bases or less.

In certain example embodiments, the training machine learning network comprises unsupervised learning, supervised learning, semi-supervised learning, reinforcement learning, transfer learning, incremental learning, curriculum learning, learning to learn, contrastive learning, or any combination thereof.

Described in certain example embodiments herein are computer program products, comprising a non-transitory computer-readable storage device having computer-executable program instructions embodied thereon that when executed by a computer cause the computer to identify or design cis-regulatory elements with cell-type, cell state, tissue type, and/or environment specific activity, the computer-executable program instructions comprising (a) computer-executable program instructions to receive, by one or more computing devices, one or more nucleic acid sequences; (b) computer-executable program instructions to transfer, by one or more computing devices, the one or more nucleic acid sequences to a deployed machine learning network; (c) computer-executable program instructions to process the one or more nucleic acid sequences with the deployed machine learning network, the deployed machine learning network generated and deployed from a training machine learning network trained on CRE-activity from a massively parallel reporter assay (MPRA) data set that provides empirical cell, tissue, or environment specific and non-specific MPRA CRE-activity measurements to the model, (d) computer-executable program instructions to generate, by the deployed machine learning network, a prediction of the CRE activity of the one or more nucleic acid sequences; and (e) computer-executable program instructions to transmit, by one or more computing devices, the predicted CRE activity to a user device associated with a user.

In certain example embodiments, the CRE activity is cell type, cell state, tissue type, or environment specific MPRA CRE-activity.

In certain example embodiments, the one or more nucleic acid sequences is a genome or a portion thereof or an epigenome or portion thereof.

In certain example embodiments, the one or more nucleic acid sequence is a DNA sequence generated from a suitable DNA sequence generation algorithm, optionally evolutionary, probabilistic, simulated annealing, or gradient based updates with random momentum (GRUM).

In certain example embodiments, processing further comprises iterative cell, tissue, or environment specific regulatory optimization of the one or more nucleic acid sequence, wherein iterative cell, tissue, or environment specific regulatory optimization comprises sequentially modifying the nucleic acid sequence in each iteration.

In certain example embodiments, processing further comprises passing the prediction to a cell, tissue, or environment specific regulatory optimizing objective function that maximizes cell specific regulatory activity.

In certain example embodiments, the cell specific regulatory optimizing objective function maximizes the predicted expression of a given sequence in one cell type, cell state, tissue type, or environment while reducing expression in all other cell types, cell states, tissue types, or environments.

In certain example embodiments, the computer program product further comprises updating the one or more nucleic acid sequences in each iteration based on the output of the cell, tissue, or environment specific regulatory optimizing objective function.

In certain example embodiments, the objective function prioritizes nucleic acid sequences with cell type, cell state, tissue type, or environment specific promoter activity, enhancer activity, silencer activity, or insulator activity.

In certain example embodiments, the cell type, cell state, tissue type, or environment specific regulatory activity comprises promoter activity, enhancer activity, silencer activity, or insulator activity.

In certain example embodiments, the machine learning network comprises a neural network, Bayesian network, random forest, matrix factorization, hidden Markov model, support vector machine, K-means clustering, K-nearest neighbor, linear classifiers, logistic classifiers, or any combination thereof.

In certain example embodiments, the neural network comprises deep learning, a convolutional neural network, or a recurrent neural network.

In certain example embodiments, the neural network comprises the convolutional neural network.

In certain example embodiments, the cell, tissue, or environment specific CRE-activity MPRA data set is obtained from a suitable database, optionally CREs centered on variants from the UK Biobank and/or GTEx.

In certain example embodiments, the cell type, cell state, tissue type, or environment specific CRE-activity MPRA data set comprises a plurality of pairs of reference and alternate alleles.

In certain example embodiments, the cell, tissue, or environment specific engineered CREs are cell type, cell state, tissue type, or environment specific engineered CREs.

In certain example embodiments, the cell type, cell state, tissue type, or environment specific CRE-activity MPRA data set was generated using vertebrate cells or invertebrate cells.

In certain example embodiments, the cell type, cell state, tissue type, or environment specific CRE-activity MPRA data set was generated using mammalian, avian, reptilian, fish, or amphibian cells.

In certain example embodiments, the cell type, cell state, tissue type, or environment specific CRE-activity MPRA data set was generated using human or non-human primate cells.

In certain example embodiments, the cell type, cell state, tissue type, or environment specific CRE-activity MPRA data set was generated using plant cells.

In certain example embodiments, the one or more nucleic acid sequence is 200 bases or less.

In certain example embodiments, the training machine learning network comprises unsupervised learning, supervised learning, semi-supervised learning, reinforcement learning, transfer learning, incremental learning, curriculum learning, learning to learn, contrastive learning, or any combination thereof.

Described in certain example embodiments herein are cis-regulatory elements (CREs), wherein the CREs are identified or designed using a computer implement method, system, and/or computer program products, optionally wherein the CRE is an engineered CRE.

In certain example embodiments, the CRE comprises two or more CREs designed or using a computer implement method, system, and/or computer program products, optionally where one or more of the two or more CREs are an engineered CRE.

In certain example embodiments, the engineered or identified CRE is cell type, cell state, tissue type, and/or environment specific.

In certain example embodiments, the engineered CRE does not have a significant match in a genome of an organism. In certain example embodiments, the organism is a vertebrate or invertebrate. In certain example embodiments, the organism is a mammal, avian, reptile, fish, or amphibian. In certain example embodiments, the organism is a human or non-human primate. In certain example embodiments, the organism is a plant.

In certain example embodiments, the CRE is specific for a diseased or abnormal cell type and/or cell state.

Described in certain example embodiments herein are engineered therapeutic polynucleotide comprising a CRE, optionally an engineered CRE, of any one of the preceding claims; and a therapeutic polynucleotide, wherein the CRE is operatively coupled to the therapeutic polynucleotide.

In certain example embodiments, the therapeutic polynucleotide (a) comprises a replacement gene; (b) encodes a therapeutic gene product; (c) comprises or encodes a genetic modification system or component thereof; (d) comprises or encodes an RNAi molecule; (e) comprises or encodes an aptamer; or (f) any combination of (a)-(e).

Described in certain example embodiments herein engineered reporter polynucleotides comprising a CRE, optionally an engineered CRE and a reporter polynucleotide, wherein the reporter polynucleotide is operatively coupled to the CRE.

In certain example embodiments, expression of the reporter polynucleotide produces a detectable signal.

In certain example embodiments, the reporter polynucleotide (a) encodes a reporter gene product; (b) comprises or encodes a genetic modification system or component thereof; (c) comprises a transcribable barcode; (d) comprises a DNA barcode; (e) comprises a target sequence for a sequence-specific binding molecule or system; (f) comprises a DNA origami reporter system or a component thereof; (g) comprises or encodes an RNAi molecule; (h) comprises or encodes an aptamer; or any combination of (a)-(h).

Described in certain example embodiments herein are vectors and vector systems that comprise one or more CREs of the present invention.

Described in certain example embodiments herein are vectors and vector systems that comprise one or more engineered therapeutic polynucleotides of the present invention and/or an engineered reporter polynucleotide of the present invention.

Described in certain example embodiments herein are delivery vehicles that comprise an engineered therapeutic polynucleotide and/or an engineered reporter polynucleotide the present invention and/or a vector or vector system of the present invention.

Described in certain example embodiments herein are cells that comprise (a) an engineered therapeutic polynucleotide and/or an engineered reporter polynucleotide of the present invention; (b) the vector or vector system of the present invention; (c) the delivery vehicle of the present invention; (d) any combination of (a)-(c).

Described in certain example embodiments herein are pharmaceutical formulations comprising a) an engineered therapeutic polynucleotide and/or an engineered reporter polynucleotide of the present invention; (b) the vector or vector system of the present invention; (c) the delivery vehicle of the present invention; (d) a cell of the present invention; or (e) any combination of (a)-(d); and a pharmaceutically acceptable carrier.

Described in certain example embodiments herein are devices configured to detect a specific cell type and/or cell state of one or more cells comprising an engineered reporter polynucleotide of the present invention and/or a delivery vehicle comprising the same.

In certain example embodiments, the device comprises microfluidic device, a lateral flow device, a tangential flow device, a normal flow device, a micro-electromechanical system, or any combination thereof.

In certain example embodiments, the device further comprises a detection reagent, wherein the detection reagent comprises a sequence-specific binding molecule or system capable of specifically binding the reporter polynucleotide, optionally at the target sequence for a sequence-specific binding molecule or system.

In certain example embodiments, the sequence-specific binding molecule or system comprises a programmable nuclease or system thereof, optionally wherein the programmable nuclease or system thereof is a Cas or Cas-based system, or an OMEGA system.

Described in certain example embodiments herein, are methods of detecting a specific cell type, cell state, tissue type, and/or environment of one or more cells in a sample comprising delivering to one or more cells an engineered reporter polynucleotide of the present invention and/or a delivery vehicle comprising the same under conditions sufficient for expression of the engineered reporter polynucleotide, wherein expression of the reporter polynucleotide occurs substantially only in the specific cell type, cell state, tissue type, and/or environment in which the CRE is active in.

In certain example embodiments, expression of the reporter polynucleotide generates a detectable signal.

In certain example embodiments, the method further comprises contacting the one or more cells with a detection reagent, wherein the detection reagent comprises a sequence-specific binding molecule or system capable of specifically binding the reporter polynucleotide, optionally at the target sequence for a sequence-specific binding molecule or system.

In certain example embodiments, the sequence-specific binding molecule or system comprises a programmable nuclease or system thereof, optionally wherein the programmable nuclease or system thereof is a Cas or Cas-based system, an IscB or IscB system, or an OMEGA system.

In certain example embodiments, binding of the sequence-specific binding molecule or system to specifically binding the reporter polynucleotide produces a detectable signal.

In certain example embodiments, the method further comprises detecting the detectable signal.

In certain example embodiments, the detectable signal indicates a specific cell type, cell state, tissue type, and/or environment.

In certain example embodiments, the detectable signal is an optical signal, a genetic perturbation, a change in gene expression of a target gene, expression of a barcode, change in genotype, change in phenotype, or any combination thereof.

In certain example embodiments, detection comprises optical detection of the detectable signal, DNA sequencing, RNA sequencing, a hybridization-based gene expression analysis, mass-spectrometry, immunodetection, or any combination thereof.

In certain example embodiments, detection comprises a single-cell resolved assay.

In certain example embodiments, the sample comprises a biofluid optionally selected from saliva, urine, blood or portion thereof, sweat, milk, semen, lymph, mucus, or feces.

In certain example embodiments, the sample comprises a tissue or portion thereof.

In certain example embodiments, the method comprises in situ spatial detection of expression of the reporter polynucleotide.

In certain example embodiments, one or more of the steps of the method are performed in vitro, in vivo, in situ, or ex vivo.

Described in certain example embodiments herein are methods of cell type, cell state, tissue type, and/or environment specific delivery of a therapeutic polynucleotide comprising delivering to one or more cells an engineered therapeutic polynucleotide of the present invention, a delivery vehicle comprising the same, or a pharmaceutical formulation thereof under conditions sufficient for expression of the engineered reporter polynucleotide.

In certain example embodiments, expression of the therapeutic polynucleotide occurs substantially only in the specific cell type, cell state, tissue type, and/or environment in which the CRE is active in.

In certain example embodiments, delivering occurs in vivo or ex vivo.

In certain example embodiments, the one or more cells are present in a subject in need thereof.

In certain example embodiments, delivery is systemic or local.

In certain example embodiments, the one or more cells are delivered to a subject in need thereof after delivering to the one or more cells an engineered therapeutic polynucleotide of the present invention, a delivery vehicle comprising the same, or a pharmaceutical formulation thereof.

In certain example embodiments, the one or more cells allogenic to the subject in need thereof or are autologous.

Described in certain example embodiments herein are methods of treating a disease or disorder or a symptom thereof in a subject in need thereof comprising delivering to one or more cells of the subject in need thereof an engineered therapeutic polynucleotide of the present invention, a delivery vehicle comprising the same, or a pharmaceutical formulation thereof under conditions sufficient for expression of the engineered reporter polynucleotide.

In certain example embodiments, expression of the therapeutic polynucleotide occurs substantially only in the specific cell type, cell state, tissue type, and/or environment in which the CRE is active in.

In certain example embodiments, delivering occurs in vivo or ex vivo.

In certain example embodiments, delivery is systemic or local.

In certain example embodiments, the method further comprises delivering the one or more cells to the subject in need thereof after delivering to the one or more cells an engineered therapeutic polynucleotide of any one of claims 78-79, a delivery vehicle comprising the same, or a pharmaceutical formulation thereof.

In certain example embodiments, the therapeutic polynucleotide (a) generates one or more genetic or epigenetic mutations, (b) generates a replacement gene product, (c) modulates gene and/or gene product expression, (d) kills or inhibits the growth or infection by a pathogen, (e) modulates one or more cellular activities, functions, or interactions, (f) kills or inhibits cell growth, differentiation, and/or proliferation, or (g) any combination of (a)-(f) in/of the one or more cells in which the therapeutic polynucleotide is expressed.

In certain example embodiments, the one or more cells comprises or consists of vertebrate cells or invertebrate cells.

In certain example embodiments, the one or more cells comprises or consists of mammalian, avian, reptilian, fish, amphibian cells, or insect cells.

In certain example embodiments, the one or more cells comprises or consists of human or non-human primate cells.

In certain example embodiments, the one or more cells comprises or consists of plant cells.

In certain example embodiments, the one or more cells comprises or consists of prokaryotic cells.

In certain example embodiments, the subject in need thereof is a vertebrate or invertebrate.

In certain example embodiments, the subject in need thereof is a mammal, avian, reptile, fish, amphibian, or insect.

In certain example embodiments, the subject in need thereof is a human or non-human primate.

In certain example embodiments, the one or more cells comprises or consists of plant cells.

These and other aspects, objects, features, and advantages of the example embodiments will become apparent to those having ordinary skill in the art upon consideration of the following detailed description of example embodiments.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings of which:

FIG. 1A-1B—Malinois training summary and test set performance. (FIG. 1A) Schematic of the experimental and modeling strategy. On the left-hand side, MPRA is used to measure CRE activity for many pairs of reference (inverted triangles) and alternate (circles) alleles. For each alt/ref pair, allelic skew is reported as the difference between these values. On the right-hand side, deep learning models are trained to predict MPRA activity directly from digitized (i.e., one-hot encoded) DNA sequences. These models can now predict allelic skew for arbitrary variants without additional experiments. (FIG. 1B) Accuracy of Malinois when predicting MPRA activity on test sequences (i.e., held out from training) from Chromosomes 7 and 13. Accuracy was measured for predictions in K562, HepG2, and SK-N-SH.

FIG. 2A-2D—Concordance of Malinois predictions with an MPRA tiling of the GATA1 locus in K562. (FIG. 2A). Summary of the genomic interval examined by an MPRA tiling screen centered on GATA1 [95], displaying genes and chromatin accessibility as measured by DHS [95]. (FIG. 2B). Aggregate summary of Malinois prediction accuracy compared to the experimental screen. (FIG. 2C-2D). Zoom-ins of the left (FIG. 2C) and right (FIG. 2D) highlighted regions in FIG. 2A. DHS (top), Malinois (middle), and MPRA (bottom) signals are strongly correlated in these regions.

FIG. 3A-3C—Malinois signals compared to DHS, H3K27ac, and STARR-seq data from ENCODE [95]. (FIG. 3A) Distribution of per-chromosome Pearson's correlation coefficients of Malinois with DHS or H3K27ac signal tracks. (FIG. 3B) Distribution of maximum Malinois signal inside of annotated peaks compared to nearest matched signal outside of peaks (Welch's t-test, p≤10-300 for all 3 peak sets). (FIG. 3C) DeepTools analysis of Malinois, STARR-seq, DHS, and H3K27ac signals in K562 at all DHS peaks annotated in K562 at Chomosome 7. The line plots represent signal averages over DHS peaks while the heatmaps display signals at individual peaks. The dip in H3K27ac signal at DHS peaks is a commonly observed pattern due to a depletion of histones at open chromatin [5], [78].

FIG. 4A-4D—Malinois VEP comparison to saturation mutagenesis MPRA from the CAGI5 competition [130]. (FIG. 4A-4D) Left-hand side of every panel reports aggregate accuracy of Malinois VEP when simulating saturation mutagenesis by MPRA. Right-hand side of every panel displaces nucleotide resolution allelic skew predictions; plots are labeled by cell type used in the experiment (K562: FIG. 4A, HepG2: FIGS. 4B-4D). FIG. 4A-4C correspond to experiments done on the PKLR, F9, LDER promoters. FIG. 4D reports an experiment done on a SORTI enhancer.

FIG. 5A-5C—Malinois and Enformer VEP performance on UKBB and GTEx variants [134]. (FIG. 5A) Accuracy of Malinois VEPs for three cell types (K562: left, HepG2: middle, SK-N-SH: right) against the UKBB/GTEx variant test set. (FIG. 5B) Accuracy of Enformer [6], the state-of-the-art chromatin state model, for VEP on the same test set as FIG. 5A. (FIG. 5C) Precision-recall for correct directional identification of variants with empirical absolute skew 0.5 using Malinois and Enformer (K562: upper curve, HepG2: middle curve, SK-N-SH: lower curve).

FIG. 6A-6D—Analysis of large databases of germline and cancer variation in humans. (FIG. 6A) Malinois predicted allelic skew distribution for all gnomAD variants; variants are separated based on overlap with evolutionarily constrained loci (phyloP [49]≥2.0). Variants in constrained loci are predicted to exert significantly larger impacts on CRE activity (Welch's t-test, p≤10-300 for all 3 cell types). (FIG. 6B) Enrichment of variants with large predicted skews (i.e., absolute allelic skew >1.0) in evolutionarily constrained loci. Enrichment odds ratio is reported for all variants (low-opacity bars) and for variants overlapping with DHS peaks in the corresponding cell type (high-opacity). (FIG. 6C) Enrichment of observed variation in Cancer Gene Census Hallmark (CGCH) gene promoters based on predicted CRE activity. Enrichment increases in regions of high predicted CRE activity. FIG. 6D) Enrichment of observed variation in Cancer Gene Census Hallmark (CGCH) gene promoters based on predicted allelic skew in predicted active CREs. Values are normalized by baseline enrichment in predicted strong CREs (i.e., predicted activity ≥1.0).

FIG. 7A-7B—Schematic of CRE sequence engineering process. (FIG. 7A) (SEQ ID NO: 1) Sequences can be iteratively updated to optimize for a predicted function. (FIG. 7B) Example of predicted activity distributions of 4000 random sequences subjected to in silico optimization of cell type specific (CTS) enhancer activity, before and after.

FIG. 8A-8C—Malinois prediction accuracy on engineered sequences. (FIG. 8A) Malinois prediction accuracy for synthetic se-quences in three cell types (Pearson's; K562: r=0.86, HepG2: r=0.76, SK-N-SH: r=0.86). Predicted and observed activity values are clamped within the range [−4, 10] for plotting purposes only. (FIG. 8B) Accuracy of Malinois predictions of entropy computed from predicted activities in each cell type (Pearson's r=0.58); low entropy corresponds to high CTS. (FIG. 8C) Distribution of absolute error in model predictions.

FIG. 9A-9B—Summary of empirical cell type specificity of synthetic sequences. (FIG. 9A) Entropy distribution for each subset of the library. (FIG. 9B) Frequency of observing sequences with entropy H≤0.2.

FIG. 10—Accuracy of GC content as a predictor of CRE activity in MPRA. (top row) GC analysis of test set [134]. (bottom) GC analysis of GATA1 tiling screen.

FIG. 11—Comparison of Malinois predictions in HepG2 and SK-N-SH with DHS signal in the corresponding cell type [95].

FIG. 12A-12B—Deep learning can accurately model cis-regulatory activity of DNA.

FIG. 13A-13E—Malinois design of cell-specific enhancers.

FIG. 14A-14F—Design of synthetic CREs drive desired cell-type specific activity in-vivo.

FIG. 15—A block diagram depicting a portion of a communications and processing architecture of a typical system to acquire one or more nucleic acid sequences from a user or database and perform machine learning resulting in predicted CRE activity, in accordance with certain examples of the technology disclosed herein.

FIG. 16—A block flow diagram depicting methods to identify or design cis-regulatory elements with cell-type, cell state, tissue type, and/or environment specific activity, in accordance with certain examples of the technology disclosed herein.

FIG. 17—A block diagram depicting a computing machine and modules, in accordance with certain examples of the technology disclosed herein.

FIG. 18A-18F—Malinois accurately predicts transcriptional activation by CREs in episomal reporters. (FIG. 18A) Schematic showing non-coding cis-regulatory elements (CREs) in the genome drive gene expression and contribute to cell type specific expression. (FIG. 18B) Overview of how MPRAs enable targeted functional characterization of hundreds of thousands of CREs on transcription in episomal reporters, and can quantify the impact of programmable 200-bp oligonucleotide sequences. MPRAs across multiple cell types enables discovery of cell type-specific activity of CREs. (FIG. 18C) (SEQ ID NO: 2) Schematic showing how deep learning enables modeling of cell type-specific CRE effects directly from nucleotide sequence. Malinois, a deep convolutional neural network, predicts CRE activity in K562 (teal, as represented in greyscale), HepG2 (yellow, as represented in greyscale), and SK-N-SH (red, as represented in greyscale). Contribution scores can be extracted from the model to determine how subsequences drive predicted function in each cell type. (FIG. 18D) Malinois predictions are highly correlated with empirically measured MPRA activity across K562 (teal, as represented in greyscale), HepG2 (yellow, as represented in greyscale), and SK-N-SH (red, as represented in greyscale). Performance for each cell type was measured using Pearson correlation (r) on a test set of sequences withheld from training. Each point corresponds to empirical and predicted activity of a single CRE in the corresponding cell type, and topological lines indicate point density (16.7%, 33.3%, 50%, 66.7%, 83.3%) in the scatter plots. Train/test splits were defined by chromosomes. (FIG. 18F) Malinois activity predictions for sequences centered on K562-specific DHS peaks activate transcription in K562. This pattern of activation is concordant with quantitative signals measured using STARR-seq, DHS-seq, and H3K27ac seq. (FIG. 18E) Malinois predictions recapitulate an MPRA screen of overlapping fragments derived from a 2.1 Mb window centered on the GATA1 gene (Pearson's r=0.91; FIGS. 24A-24D). Purple signal, as represented in greyscale, indicates overlapping signal while blue and red signal, as represented in greyscale, indicate either higher activity measurements or predictions by MPRA or Malinois, respectively, in the window chrX: 48,000,000-49,000,000.

FIG. 19A-19E—CODA effectively designs novel cell type-specific CREs using Malinois predictions. (FIG. 19A) CODA designs synthetic elements by iteratively updating sequences to improve predicted function. Cell type-specific CRE activity of all 200 bp DNA oligos induces a topology over a massive sample space. CODA initializes sequences in this space and uses Malinois to predict local topology. An objective function is used by CODA to direct updates of sequences to move as desired through predicted topology. Updated sequences can be further modified in silico until a stopping criteria is reached and final candidates are proposed for experimental validation. (FIG. 19B) Composition of the MPRA library designed to empirically evaluate candidate cell type-specific CREs. A total of 75,000 sequences were selected from the human genome (green hues, as represented in greyscale) or designed ab initio using CODA (purple hues, as represented in greyscale) to maximize the MinGap score for a target cell type. Aggregated natural and synthetic sequences are indicated by blue and coral coloring as represented in greyscale, respectively. Sequences generated using motif-penalization are delineated by the dotted overlay. (FIG. 19C) Computationally-designed CREs maintain high transcriptional activity in target cells while improving silencing in off-target cells. The three rows of box plots correspond to candidate CREs intended to drive cell type-specific expression in K562, HepG2, and SK-N-SH. Each group of three boxes indicate the distribution of MPRA log₂ fold change (log 2FC) measurements in K562 (teal, as represented in greyscale), HepG2 (yellow, as represented in greyscale), and SK-N-SH (red, as represented in greyscale) for a set of sequences nominated by the indicated design strategy on the x-axis. Boxes demarcate the 25th, 50th, and 75th percentile values, while whiskers indicate the outermost point with 1.5 times the interquartile range from the edges of the boxes. Sequences with a replicate log 2FC standard error greater than 1 in any cell type were not included. (FIG. 19D) CODA-designed synthetic sequences achieve higher overall cell type-specific activity than natural sequences. Box plots display distribution of MinGap scores to quantify cell-specific CRE function and color indicates intended target cell type (K562: teal, as represented in greyscale; HepG2: yellow, as represented in greyscale; SK-N-SH: red, as represented in greyscale). Boxes demarcate the 25th, 50th, and 75th percentile values, while whiskers indicate the outermost point with 1.5 times the interquartile range from the edges of the boxes. Sequences with a replicate log 2FC standard error greater than 1 in any cell type were not included. (FIG. 19E) Top row: propeller plots for each sequence group. The radial distance corresponds to the distance between the maximum and minimum cell type activity values, while the angle of deviation from an axis quantifies the relative activity of the highest off-target cell type (Methods). Teal, yellow, and red areas, as represented in greyscale, represent sequences in which the MinGap:MaxGap ratio is greater than 0.5. Dot shading are associated with the activity in the minimum off-target cell type. Bottom row: percentages of points in each delimited area rounded to the nearest integer. The point count in the center represents sequences with quasi-uniform activity across cell types, while the gray wedges count sequences with a low MinGap. The groups synthetic and synthetic-penalized were randomly sub-sampled to match the size of the two natural groups (see FIG. 40 for full plots).

FIG. 20A-20E—Interpreting CRE syntax in engineered elements. (FIG. 20A) (SEQ ID NO: 3) Malinois contribution scores enable nucleotide resolution interpretation of sequence activity. Shown is a representative synthetic CRE designed to drive HepG2-specific reporter expression. Enriched motifs are demarcated on the upper sequence track. Contribution scores are plotted for each cell type on the lower track (K562: teal, as represented in greyscale, HepG2: yellow, as represented in greyscale, SK-N-SH: red, as represented in greyscale). Positive and negative values indicate segments contribute to transcriptional activation or silencing, respectively, in the corresponding cell type. Motifs with a strong known-motif match have the name of the match in parenthesis preceding their label (Methods). (FIG. 20B) Left heatmap: average contributions of core motifs in K562, HepG2, SK-N-SH (left to right columns). Center bar plot: motif enrichment in synthetic (light gray) and natural (dark gray) sequences. The x-axis represents the percentage of sequences in each group that contain at least one instance of that motif denoted on the y-axis. Right bar plot: motif program association derived from the NMF features matrix. Colors, as represented in greyscale correspond to programs listed in FIG. 20D. (FIG. 20C) Cooccurrences of enriched motifs are more prevalent in synthetic CREs. Co-occurrence percentage indicates the percentage of sequences in each group containing a pair of motifs (Methods; see FIG. 46A-46C for all percentages). Upper and lower triangular percentages correspond to natural and synthetic sequences respectively. Red and blue motif labels, as represented in greyscale, denote motifs with mostly positive or negative contribution, respectively. (FIG. 20D) Specific functional programs drive cell type-specific transcription. Empirical program function calculated using a weighted average of MPRA log 2FC scores based on topic mixture displayed in FIG. 20C. Ten cell type specificity-driving programs were identified using the same criteria applied to identify cell type-specific sequences (bright colored points, as represented in geryscale; 1 for K562, 2 for HepG2, 2 for SK-N-SH). Seven programs are not associated with cell type-specific transcription (pastel points). Program 11 is overplotted by program 8 and program 4 partially obstructs program 9 on the propeller plot. (FIG. 20E) Synthetic and natural sequences show distinct patterns of higher order arrangements of TF binding motifs. Colored bar plots, as represented in greyscale, generated from NMF decomposition of synthetic and natural sequences based on enriched motif content reveal the functional programs used in each sequence. For each sequence, programs colored based on the key in FIG. 20D and are plotted as a fraction of total program content. Note, in a few cases, sequences were not assigned to any program with any frequency yielding a blank bar. Line plots display MPRA log 2FC scores for the above sequences in K562 (teal, as represented in greyscale), HepG2 (yellow, as represented in greyscale), and SK-N-SH (red, as represented in greyscale). Sub-panels are organized into rows by expected target cell type and columns by method used to nominate candidate sequences. Sequences in each panel are sorted by hierarchical clustering based on program content.

FIG. 21A-21H—In vivo validation of synthetic elements using zebrafish and mouse. (FIG. 21A) Prioritization workflow for selecting cell specific CREs for in vivo validation. (FIG. 21B) A synthetic liver-specific CRE drives transgene expression in the larval zebrafish liver. Brightfield, GFP, and merged whole animal imaging 96 hours post-fertilization indicates that the synthetic CRE reproducibly drives transgene expression in zebrafish liver (white arrows). Lateral view, anterior to the left, dorsal up. (FIG. 21C) CODA-designed SK-N-SH-specific CRE drives GFP expression in embryonic zebrafish neurons (white arrows). Brightfield, GFP, and merged imaging of the brain and anterior spinal region of animals 48 hours post-fertilization show transgene expression in the developing brain and spinal cord. Embryo 2 shows additional incidental off-target expression in vascular tissue. Lateral view, anterior to the left, dorsal up. (FIG. 21D) Synthetic SK-N-SH-specific CRE drives transgene expression in 5-week-old postnatal mice. X-Gal staining for LacZ of the medial section of the brain reveals specific transgene expression at layer 6 of the neocortex. (FIG. 21E) LacZ expression in deep cortical layers is neuron-specific. Top panel: representative confocal images of layer 6 neurons, microglia, astrocytes, and merged image demonstrating the absence of transgene in control mice. Lower panel: confocal images show that transgene expression is exclusive to cortical neurons with arrows indicating colocalization between LacZ signal and neurons. Scale bars: 20 um. (FIG. 21F) Box plot showing proportion of neurons, astrocytes, and microglia positive for the transgene. Neurons exclusively express LacZ. ****: adj p<0.0001 for Kruskal-Wallis one-way ANOVA. (FIG. 21G) Synthetic N1 CRE drives specific transgene expression in the brain. LacZ expression by synthetic N1 CRE is measured using RNA-seq and normalized by the expression of LacZ in mice transgenic for the minP empty vector. (FIG. 21H) Nucleotide level effects of synthetic neuronal CRE N1. Top track: Malinois contribution scores reveal the role of ETS and CREB-like binding domains in mediating synthetic CRE activity in neurons. Subsequences of high predicted contribution to SK-N-SH activity overlap with ETS- and CREB-like binding motifs based on visual inspection. Bottom track: Single nucleotide effects measured experimentally using MPRA saturation mutagenesis. Circular points represent the expression change measure by MPRA when only that position is mutated in N1. Letters represent the reference nucleotide of the N1 sequence at that position with the height corresponding to the mean expression change at that position with opposite sign.

FIG. 22—MPRA library reproducibility. Scatter plots compare the log₂ (Fold-Change) (log₂(FC)) of 20,303 sequences shared between the UKBB and GTEx MPRA libraries, two libraries experimentally conducted independently from each other at distinct points of time. The x-axis corresponds to the log₂(FC) as measured in UKBB, and the y-axis corresponds to the log₂(FC) as measured in GTEx. The Pearson's correlation coefficient is shown in the right bottom corner. Oligos with a replicate log₂(FC) standard error greater than 1 were omitted from the comparisons.

FIG. 23—Model schematic. Schematic of the Malinois model architecture. Malinois is composed of 3 convolutional layers, 1 shared linear layer, and 3 independent branches of 4 linear layers—1 branch for activity predictions in each cell type. All hidden layers are followed by rectified linear units while convolutional layers are also separated by pooling operations. Layers with weights inherited from Basset at the initiation of training are indicated.

FIG. 24A-24D—Bayesian optimization effectively finds reasonable hyperparameter settings. (FIG. 24A) Validation and test set performance of models from hyperparameter proposals picked by Bayesian Optimization, in order. Dotted lines indicate test set performance of Malinois. (FIG. 24B) Transfer 1 earning by initializing weights from Basset results in less variation and overall improvement in training outcomes. (FIG. 24C) Duplicating and augmenting the training data by taking the reverse compliments of the input sequences improves modeling accuracy. (FIG. 24D) Replacing fully-connected layers in the decoder segment of CNNs increases variance in fitted model performance, although the top performing branched decoder models show improvement comparatively.

FIG. 25A-25C—Cell type accuracy of model. (FIG. 25A) Cross cell-type activity comparisons between empirical measurements and Malinois predictions organize and correlate similarly to empirical-to-empirical comparisons. Top scatter plots: empirical vs empirical cross-cell-type log₂(FC). Bottom scatter plots: empirical vs predicted cross-cell-type log₂(FC). Pearson correlation coefficients are shown in the left-bottom corner of each scatter plot. (FIG. 25B) Malinois can be used to identify highly active cell type-specific CREs. MinGap scores calculated using Malinois predictions correlate well with MPRA MinGap measurements for sequences in the held-out test set. Points are colored based on correct prediction of maximally active cell type by Malinois. (FIG. 25C) Malinois predictions of cell type associated with maximum CRE function are more accurate for sequences with high empirical specificity. Stacked bar plot displaying number of sequences in the test set falling into discrete bins based on an empirically measured MinGap threshold. Lower boundary of each bin is indicated on the x-axis and hue delineates sequences that are categorized correctly (dark grey) or incorrectly (light gray).

FIG. 26A-26B—Correlation of Malinois predictions and empirical MPRA tiling data. (FIG. 26A) Malinois predictions are highly correlated with empirical MPRA measurements of tiled sequences in the GATA locus (chrX: 47,785,602:49,880,397)^{5, 48-50} in K562 (Pearson's r=0.91, Spearman's p=0.84). X-axis and y-axis correspond to empirical measurements and Malinois predictions, respectively for oligos in the library (n=51242 oligos). Sequences which overlap with oligos from the validation data split used for model selection were removed from this plot and correlation calculations (n=2420 oligos omitted). Additionally, oligos with a replicate log 2FC standard error greater than 1 in any cell type were omitted from the plots. (FIG. 26B) Malinois predictions projected onto the genome are correlated with empirical MPRA projections and DHS signal in regions with active CREs. Pearson's r and Spearman's rho are calculated for the predicted track compared to either DHS (upper) or MPRA (lower).

FIG. 27A-27C—Malinois concordance with DHS/H3K27ac/STARR. (FIG. 27A) Malinois genome-wide predictions correspond well with DHS signal in HepG2. Deeptools plots of Malinois genome-wide predictions and DHS signal centered at DHS peaks in HepG2 cell lines on chromosome 13. (FIG. 27B) DHS signal and Malinois genome-wide predictions are also similar in SK-N-SH. Similar Deeptools plots to a except using SK-N-SH derived data. (FIG. 27C) Malinois genome-wide predictions are significantly associated with candidate CRE mapping (DHS-seq, and H3K27ac ChIP-seq) and orthogonal signals of CRE functional characterization (STARR-seq). Boxplots display average signal generated by Malinois genome-wide predictions within peaks annotated using DHS, H3K27ac, or STARR-seq (orange) compared to paired upstream (blue) and downstream (green) flanking regions. Boxes demarcate the 25th, 50th, and 75th percentile values, while whiskers indicate the outermost point with 1.5 times the interquartile range from the edges of the boxes. Stars indicate a significant (−log 10 p-value >100) for two t-tests comparing signals within peaks and both upstream and downstream regions outside of peaks.

FIG. 28A-28K—Screening sequence design hyperparameters for generating synthetic CREs. Different hyperparameter combinations for Fast SeqProp (FIG. 28A)-(FIG. 28F) and Simulated Annealing (FIG. 28G)-(FIG. 28K) were tested to generate predicted K562-specific synthetic CREs. Predicted log 2-fold-change, predicted minGap activity, 4-mer heterogeneity, and GC content was measured for each sequence and plotted as a function of hyperparameter choices.

FIG. 29A-29B—Example sequence generation trajectory. (FIG. 29A) Fast SeqProp can generate sequences that are predicted to minimize an objective function. A trajectory was generated for 512 sequences using 200 update steps. Top: An example trajectory of a single sequence in the trajectory. Color, as represented in greyscale, represents nucleotide identity along the sequence after each update during the algorithm (A: Green (as represented in greyscale), C: Blue (as represented in greyscale), G: Yellow (as represented in greyscale), T: Red (as represented in greyscale)). Bottom: The predicted objective value of sequences at each step of Fast SeqProp. The mean is indicated by the line and bounds of the 95 percentile data range are shaded light blue, as represented in greyscale. The example displayed above is indicated by the orange line, (as represented in greyscale). (FIG. 29B) Same as FIG. 29A, but generated using 2000 steps of simulated annealing.

FIG. 30A-30B—Motif match scores during penalization. (FIG. 30A) Motifs can be depleted from Fast SeqProp-generated sequences using motif penalization. Motif numbers on the x-axis correspond to the first round in which their matches are penalized during Fast SeqProp, as they were the top match from the previous round. For each target cell type, four independent tracks of penalization were carried out (Methods) to account for potential enrichment effects of the random initialization when generating sequences. (FIG. 30B) Underrepresented motifs are progressively enriched as preferred alternatives are depleted. Box plots capture distribution of motif matches across sequences produced in each round of penalized generation. Motif numbers on the x-axis correspond to the first round in which their matches are penalized during Fast SeqProp. Motifs are specifically depleted in rounds where they are introduced into the penalty calculation, but can gradually rise during preceding rounds. In the y-axis, the motif-presence score of each motif is calculated by summing all the motif-match scores that pass a score threshold in a sequence, and dividing the sum by the score of the motif consensus sequence. Boxes demarcate the 25th, 50th, and 75th percentile values, while whiskers indicate the outermost point with 1.5 times the interquartile range from the edges of the boxes.

FIG. 31A-31C—Annotation of naturally occurring sequences. (FIG. 31A) Sequences nominated by DHS accessibility (DHS-natural) and by Malinois (Malinois-natural) were intersected with ENCODE cCREs (promoter-like sequences, proximal enhancer-like sequences, distal enhancer-like sequences, and CTCF-only) to determine overlap with existing putative regulatory elements. 94% of DHS-natural sequences intersect a cCRE while only 34.2% of Malinois-natural sequences intersect a cCRE suggesting that Malinois may exploit sequences features not captured by typical cCRE measures to select a sequence that drives cell type-specific activity. (FIG. 31B) To explore additional genomic features that may overlap DHS-natural and Malinois-natural sequences were annotated using annotatePeaks.pl from the HOMER suite. Annotations were generated for the whole genome (hg38), the DHS-natural and Malinois-natural libraries as a whole, as well as DHS-natural and Malinois-natural by individual cell type. DHS-natural and Malinois-natural largely resemble the distribution of annotations genome-wide barring an overrepresentation of simple repeats in Malinois-natural sequences driven by SK-N-SH sequences. Despite this, selected sequences seem to be a representative sample of genomic features. (FIG. 31C) DHS-natural and Malinois-natural sequences were intersected to determine overlap between naturally occurring sequences. Notably overlap was minimal between selection methods (0.10%-4.1%) depending on cell type.

FIG. 32A-32C—Predicted library activity. (FIG. 32A) Distribution of projected activity in K562 (teal, as represented in greyscale), HepG2 (gold, as represented in greyscale), and SK-N-SH (red, as represented in greyscale) for candidate CREs predicted to drive K562-specific transcription. Boxes demarcate the 25th, 50th, and 75th percentile values, while whiskers indicate the outermost point with 1.5 times the interquartile range from the edges of the boxes. (FIG. 32B) Same as FIG. 32A, but for candidate CREs predicted to drive HepG2-specific transcription. (FIG. 32C) Same as FIG. 32A and FIG. 32B, but for candidate CREs predicted to drive SK-N-SH-specific transcription.

FIG. 33A-33B—K-mer and Hamming distance. (FIG. 33A) Algorithms for model-guided sequence designs produce diverse, non-degenerate candidate CREs. Box plot displays the distribution of average Levenshtein distance to 4 nearest neighbors for sequences in categories indicated on the x-axis. As a control, we randomly selected 4000 shuffled sequences from the candidate CRE library and 19381 promoter sequences extracted from RefGene by taking the 200 nucleotides upstream of (strand aware) TSS annotations for mRNAs. Malinois-natural results are plotted on aggregate, only using non-repeat element matched sequences, and repeat element matched sequences. Spearman's correlation coefficient was calculated between penalization round number (starting at zero) and average Hamming distances to 4 nearest neighbors. Boxes demarcate the 25th, 50th, and 75th percentile values, while whiskers indicate the 1st and 99th percentile values. (FIG. 33B) Algorithms for model-guided sequence designs produce sequences with diverse, non-redundant 7-mer usage. Plot is the same as a except it displays average L1 distance of 7-mer content between sequences and 4 nearest neighbors, divided by 2. Boxes demarcate the 25th, 50th, and 75th percentile values, while whiskers indicate the 1st and 99th percentile values.

FIG. 34A-34I—Variation in 4-mer content between natural and synthetic cell type specific elements. (FIG. 34A) L1 distance between groups of designed CREs based on marginalized 4-mer frequencies in each group. (FIG. 34B) UMAP embedding of all non-penalized CREs in the designed cell type specific sequence element library colored by synthetic (pink, as represented by greyscale) or natural (blue, as represented by greyscale) provenance. (FIG. 34C) 12,000 random 200-mers embedded in the same UMAP as (FIG. 34A). (FIG. 34D) The subset of points in (FIG. 34A) that are natural CREs selected to be cell type specific based on DHS or Malinois predictions, colored (shaded) by target cell type. (FIG. 34E) A kernel density estimate from the natural CREs in (FIG. 34D) but recolored (reshaded) by if the element was selected using DHS (orange, as represented by greyscale) or Malinois (green, as represented by greyscale). (FIG. 34F) The subset of points in (FIG. 34A) that are synthetic CREs, colored (shaded) by target cell type. (FIG. 34G) A kernel density estimate from synthetic CREs designed by Fast SeqProp, colored (shaded) by target cell type. (FIG. 34H) Same as (FIG. 34G) except from CREs designed by Simulated annealing. (FIG. 34I) Same as (FIG. 34G) except CREs designed by AdaLead. The UMAP region containing 90% of random sequences is indicated by a gray line in (FIG. 34D)-(FIG. 34I).

FIG. 35—MPRA measurements for individual elements are reproducible between different experiments and libraries. MPRA activity measurements made in the training data plotted on the x-axis are highly correlated with later measurements made in the CODA library on the y-axis. Measurements were made in K562 (teal, as represented in greyscale), HepG2 (gold, as represented in greyscale), and SK-N-SH (red, as represented in greyscale).

FIG. 36A-36C—Library prediction validation plots. (FIG. 36A) Prospective Malinois predictions of candidate cell type-specific CRE activity is correlated with experimental measurements across all three tested cell types. The scatter plot corresponds to predictions and measurements made in K562. Solid contour lines demarcate 95% density of points corresponding to candidate CRE expected to drive expression in K562. Dotted contour lines indicate 95% density of CREs expected to drive specific expression in one of the other two cell types. Color (shading) indicates sequence selection or generation method. One-dimensional density estimates along axes share the same line style and color (greyscale) associations. Sequences with a replicate log 2FC standard error greater than 1 in any cell type were omitted from the plots. (FIG. 36B) Same as FIG. 36A, but in HepG2. (FIG. 36C) Same as FIG. 36A, but in SK-N-SH.

FIG. 37—Granular Malinois prediction performance of CODA library. Pearson correlation coefficient values between Malinois activity predictions and MPRA empirical measurements in K562 (teal, as represented in greyscale), HepG2 (gold, as represented in greyscale), and SK-N-SH (red, as represented in greyscale) of the CODA library broken down by method group.

FIG. 38A-38C—Empirical library activity. (FIG. 38A) Empirical log₂(Fold-Change) activity measured in K562 (teal, as represented in greyscale), HepG2 (gold, as represented in greyscale), and SK-N-SH (red, as represented in greyscale) for sequences targeting K562 binned by design method group. Boxes demarcate the 25th, 50th, and 75th percentile values, while whiskers indicate the outermost point with 1.5 times the interquartile range from the edges of the boxes. (FIG. 38B) Same as (FIG. 38A) except sequences targeting HepG2. (FIG. 38C) Same as (FIG. 38A) except sequences targeting SK-N-SH.

FIG. 39A-39C—Library MinGap. (FIG. 39A) Malinois improves identification of CREs with K562-specific activity and synthetic sequence generation enables creation of CREs with enhanced functions. Distribution of MPRA-measured K562-specific activity in various candidate CRE groups. Green and aquamarine lines, as represented in greyscale, indicate median MinGap of DHS-natural and Malinois-natural candidates respectively. Sequences with a replicate log 2FC standard error greater than 1 in any cell type were omitted from the plots. Boxes demarcate the 25th, 50th, and 75th percentile values, while whiskers i ndicate the outermost point with 1.5 times the interquartile range from the edges of the boxes. (FIG. 39B) Same as (FIG. 39A) except quantification of candidate sequences targeting HepG2. (FIG. 39C) Same as (FIG. 39A) except quantification of candidate sequences targeting SK-N-SH.

FIG. 40—Complete propeller plots. Propeller plots of refined synthetic subsets of the library (see FIG. 19E legend for description of coordinate system).

FIG. 41—Cell type activity comparisons. Scatter plots comparing empirical log₂(Fold-Change) activity in each pair of cell types for each design group. Color, as represented in greyscale, indicates the target cell type for which sequences were designed (synthetic) or selected (natural).

FIG. 42A-42F—Contribution block ablation. (FIG. 42A) Predicted activity (labeled as initial) in K562 (teal, as represented in greyscale), HepG2 (gold, as represented in greyscale), and SK-N-SH (red, as represented in greyscale) of the library sequences targeting K562. Activity predictions of disrupted sequences when ablating segments corresponding to negative (gray), positive (dark gray) contribution blocks, or outside blocks (light gray) determined by contribution scores in each cell type. The number above each box denotes the number of sequences for which a contribution block type was found. All initial activity boxes correspond to 25,000 sequences. Boxes demarcate the 25th, 50th, and 75th percentile values, while whiskers indicate the outermost point with 1.5 times the interquartile range from the edges of the boxes. (FIG. 42B) Same as (FIG. 42A) but library sequences targeting HepG2. (FIG. 42C) Same as (FIG. 42A) but library sequences targeting SK-N-SH. (FIG. 42D) Distributions denoting the number of positions disrupted in (FIG. 42A) by negative (gray), positive (dark gray) contribution blocks, or outside blocks (light gray). Boxes demarcate the 25th, 50th, and 75th percentile values, while whiskers indicate the outermost point with 1.5 times the interquartile range from the edges of the boxes. (FIG. 42E) Same as (FIG. 42D) but disrupted in (FIG. 42B). (FIG. 42F) Same as (FIG. 42D) but disrupted in (FIG. 42C).

FIG. 43A-43D—Predicted functionality of core motifs. (FIG. 43A) Information-Content logos of core motifs. The x-axis and y-axis denote positions and bits, respectively. (FIG. 43B) Matches to known human TF binding motifs in JASPAR or HOCOMOCO. An asterisk at the beginning of the name indicates a moderate match with 1<E-value <10. No name (dashes) indicates that any possible match had an E-value <10. Otherwise, the name corresponds to a match with an E-value <1. The symbols +/− at the end of the name indicate the orientation of the match as forward or reverse complement respectively. (FIG. 43C) Activity predictions of sequences consisting of randomly sampled motif instances in the center and randomly background-sampled flanks in K562 (teal, as represented in greyscale), HepG2 (gold, as represented in greyscale), and SK-N-SH (red, as represented in greyscale), along with activity predictions of fully random background-sampled sequences in K562, HepG2, and SK-N-SH (all in light gray). Boxes demarcate the 25th, 50th, and 75th percentile values, while whiskers indicate the outermost point with 1.5 times the interquartile range from the edges of the boxes. (FIG. 43D) Predicted activity effect of disrupting all motif instances in the sequence library binned my motif presence score. Teal, gold, and red boxes, as represented in greyscale, correspond to effects to the predicted activity in K562, HepG2, and SK-N-SH, respectively. The y-axis corresponds to the activity prediction of the original (undisrupted) sequences minus the activity prediction of sequences with disrupted motif instances replaced by randomly background-sampled segments. The integer n below each bin of boxes indicates the number of sequences present in each motif score bin. Boxes demarcate the 25th, 50th, and 75th percentile values, while whiskers indicate the outermost point with 1.5 times the interquartile range from the edges of the boxes.

FIG. 44A-44C—Predicted functionality of TF-MoDISco original patterns. (FIG. 44A) Logos of the patterns found by TF-MoDISco. Names of core motifs forming the pattern are written below. The symbols +/− at the end of the name indicate the orientation of the match as forward or reverse complement respectively. (FIG. 44C) Activity predictions of sequences consisting of randomly sampled motif instances in the center and randomly background-sampled flanks in K562 (teal, as represented in greyscale), HepG2 (gold, as represented in greyscale), and SK-N-SH (red, as represented in greysca), along with activity predictions of fully random background-sampled sequences in K562, HepG2, and SK-N-SH (all in light gray). Boxes demarcate the 25th, 50th, and 75th percentile values, while whiskers indicate the outermost point with 1.5 times the interquartile range from the edges of the boxes. (FIG. 44D) Predicted activity effect of disrupting all motif instances in the sequence library binned my motif presence score. Teal, gold, and red boxes correspond to effects to the predicted activity in K562, HepG2, and SK-N-SH, respectively. The y-axis corresponds to the activity prediction of the original (undisrupted) sequences minus the activity prediction of sequences with disrupted motif instances replaced by randomly background-sampled segments. The integer n below each bin of boxes indicates the number of sequences present in each motif score bin. Boxes demarcate the 25th, 50th, and 75th percentile values, while whiskers indicate the outermost point with 1.5 times the interquartile range from the edges of the boxes.

FIG. 45A-45C—Motif enrichment by cell type target. (FIG. 45A) Motif representation in K562-optimized sequences only. Bar width indicates the fraction of natural (dark gray) or synthetic (light gray) K562-optimized sequences containing the motif. (FIG. 45B) Same as (FIG. 45A) but in HepG2-optimized. (FIG. 45C) Same as (FIG. 45A) but in SK-N-SH-optimized.

FIG. 46A-46C—Motif co-occurrence percentages. (FIG. 46A) Motif co-occurrence representation in K562-optimized sequences only. Color, as represented by greyscale, indicates the fraction of natural (upper triangle) or synthetic (lower triangle) K562-optimized sequences containing a motif pair. (FIG. 46B) Same as FIG. 46A, but in HepG2-optimized. (FIG. 46C) Same as FIG. 46A, but in SK-N-SH-optimized.

FIG. 47A-47D—Type: token. (FIG. 47A) Individual synthetic sequences are composed of more unique enriched sequence motifs than natural sequences. Distribution of unique motifs (types) in each sequence, binned by CRE proposal method. Boxes demarcate the 25th, 50th, and 75th percentile values, while whiskers indicate the outermost point with 1.5 times the interquartile range from the edges of the boxes. (FIG. 47B) Synthetic sequences contain more instances of enriched motifs than natural sequences. Distribution of total motif instances (tokens) in each sequence, binned by CRE proposal method. Boxes demarcate the 25th, 50th, and 75th percentile values, while whiskers indicate the outermost point with 1.5 times the interquartile range from the edges of the boxes. (FIG. 47C) Distribution of type: token in each sequence, binned by CRE proposal method. Boxes demarcate the 25th, 50th, and 75th percentile values, while whiskers indicate the outermost point with 1.5 times the interquartile range from the edges of the boxes. (FIG. 47D) Motif penalization reduces motif redundancy in synthetic CREs. Boxplots are similar to c. except synthetic elements are broken up into more granular bins. Boxes demarcate the 25th, 50th, and 75th percentile values, while whiskers indicate the outermost point with 1.5 times the interquartile range from the edges of the boxes.

FIG. 48A-48B—Full NMF structure plot and top-motif set per program. (FIG. 48A) NMF decomposes sequence libraries and aggregates motifs into 12 distinct functional programs. Various CRE proposal methods favor distinct patterns of program usage. Top-left, grayscale heatmap: Motifs (y-axis) are identified in each sequence (x-axis). Shading indicates the number of motif matches in a sequence, capped at 5 matches. Top-right horizontal bar plot: Frequency of program association for each motif extracted from NMF feature matrix, unit normalized. Y-axis is shared with top-left and ordering was set by clustering motifs using the feature matrix. Program coloring is consistent with FIG. 20D. Bottom, vertical bar plot: Program decomposition of individual sequences, unit normalized. Bottom, colored stips: Demarcation of CRE metadata (i.e., predicted target cell type, generation method, objective function modification) with color, as represented in greyscale, corresponding to legend on the right and side. CREs are clustered within these subsets based on program content. (FIG. 48B) Raw values from the NMF feature matrix for the top 6 motifs associated with each program. Coloring (as represented in greyscale) of program subtitles is consistent with FIG. 20D.

FIG. 49A-49B—Activating, repressing, and ubiquitous program content and usage. (FIG. 49B) Marginal function of each NMF program in each cell type used to generate FIG. 20D. These functional summaries are calculated using a weighted average of motif contributions (FIG. 20B, Methods:Motif contributions) calculated using the unit normalized feature matrix from NMF (Methods). (FIG. 49B) Program content distribution for 12 programs assessed by NMF decomposition. Sequences are grouped by design methodology (x-axis) and intended target cell type (hue). Inset slider indicates average program function over K562, HepG2, and SK-N-SH (average repressive function indicated by blue (as represented in greyscale), averages clipped within +/−1 range). Boxes demarcate the 25th, 50th, and 75th percentile values, while whiskers indicate the outermost point with 1.5 times the interquartile range from the edges of the boxes.

FIG. 50A-50E—Overall program usage. (FIG. 50A) Distribution of total program coefficients for sequences in different design groups. (FIG. 50B) Heterogeneity of program coefficients for each sequence measured by entropy. (FIG. 50C) Aggregating activating program content and collapsing over cell types. (FIG. 50D) Same as FIG. 50C, except repressing programs. (FIG. 50A)-(FIG. 50D) Boxes demarcate the 25th, 50th, and 75th percentile values, while whiskers indicate the outermost point with 1.5 times the interquartile range from the edges of the boxes, outliers are indicated as points. (FIG. 50E) Simultaneous usage of activating and repressing programs and motifs is the favored strategy for synthetic sequence design. Sequences are annotated as activating if composed of at least 1/10^{ths} activating programs and are annotated as repressing if composed of similar repressing program content. The fraction of sequences in each group passing none, strictly one, or both of these criteria are plotted.

FIG. 51A-51H—MPRA models for A549 and HCT116 predict synthetic CREs. Additional MPRA measurements were made in A549 and HCT116 for 318,247 and 442,482 elements and used to model CRE activity in these cell lines, respectively. (FIG. 51A-51B) Pairplot showing distribution of activity for sequences measured in (FIG. 51A) A549 and (FIG. 51B) HCT116 and other cell types. (FIG. 51C-51D) A model trained on sequences with (FIG. 51C) A549 and (FIG. 51D) HCT116 measurements with the same settings as Malinois accurately predicts MPRA measurements of CRE function. Scatterplots show model performance on held out test data. (FIG. 51E) Predicted activity of K562-targeting CREs across 5 cell lines. CREs are separated into frames based on design methodology. Text inset indicates percentage of CREs where the intended target had the highest prediction before and after A549 and HCT116 predictions were considered. (FIG. 51F) Same as (FIG. 51E) except for HepG2-targeting CREs. (FIG. 51G) same as (FIG. 51E) and (FIG. 51F) except for SK-N-SH-targeting CREs. (FIG. 51H) On-target predicted activity of CREs summarized by minGap before and after A549 and HCT116 predictions were included in the calculation. Each frame collects CREs from the five frames to the left. Each box represents CREs from a different design method.

FIG. 52A-52E—Enformer based prioritization of oligos for in vivo tests. (FIG. 52A) Enformer can predict CRE-driven changes in epigenetic and transcription dynamics of transgenes inserted into the H11 safe harbor locus in mice. Three example sequence tracks display predicted DHS signals observed in the livers of 15.5 day old mice. Transgene transcription start site and poly-adenylation signal are indicated by the gray bars. The first track is the predicted signal when the input sequence at the CRE insertion site is all Ns. The second track is an example predicting using a validated HepG2-specific synthetic CRE. The third displays the differential DHS effect. (FIG. 52B) Empirical K562 MinGap measurements are well correlated with Enformer-predicted features of spleen-specific transcriptional activation (Methods). (FIG. 52C) Empirical HepG2 MinGap measurements are also well correlated with Enformer-predicted features of liver-specific transcriptional activation. (FIG. 52D) Empirical SK-N-SH MinGap measurements are also well correlated with Enfomer-predicted features of neural-specific transcriptional activation. (FIG. 52E) Enformer-based cell type matched tissue-specific transcriptional activation predictions (K562 matched to spleen, HepG2 matched to liver, SK-N-SH matched to adult brain). Stars indicate family-wise error rate corrected p-values <1e-4.

FIG. 53A-53F—Malinois contribution scores/Enformer/MPRA results for in vivo sequences. Collection of synthetic sequences prioritized for in vivo validation. Sequences in panels (FIG. 53A-FIG. 53C) (SEQ ID NO: 4-6) and (FIG. 53D-FIG. 53F) (SEQ ID NO: 7-9) are expected to drive expression in liver and neurons, respectively. Left column: Nucleotide sequence, motif matches, and contribution score tracks for each candidate. Right column: Bar plots of empirical MPRA signal (left y-axis) in K562 (teal, as represented in greyscale), HepG2 (gold, as represented in greyscale), and SK-N-SH (red, as represented in greyscale) as well as aggregated Enformer predictions (right y-axis) of epigenetic signals reflecting transcriptional activation in mouse spleen (dim teal, as represented in greyscale), liver (dim gold, as represented in greyscale), neural tissue (dim red, as represented in greyscale), heart, intestine, kidney, limb buds, lung, pancreas, and stomach.

FIG. 54A-54B—A synthetic CRE reproducibly drives expression in zebrafish livers. (FIG. 54A) Expression of control transgene lacking synthetic CRE fails to drive GFP expression 4 days post-fertilization. All 18 control animals fail to show GFP expression. (FIG. 54B) Synthetic CRE drives GFP expression in zebrafish livers and yolk-sacs. Synthetic CRE drives expression in zebrafish livers in 27 out of 36 animals, and yolk-sacs in 32 out of 36 animals.

FIG. 55A-55C—Additional synthetic CREs drive expression in zebrafish gastrointestinal system. (FIG. 55A) Expression of control transgene lacking synthetic CRE fails to drive GFP expression 5 days post-fertilization. All 18 control animals fail to show GFP expression. (FIG. 55B) A second synthetic HepG2-specific CRE sporadically drives GFP expression in the yolk-sac, but not the liver. 8 out of 18 animals show CRE induced expression in the yolk-sacs 5 days post fertilization. (FIG. 55C) A third synthetic HepG2-specific CRE drives expression drives GFP expression in the yolk-sac.

FIGS. 56A-56L—SK-N-SH-specific CREs drive expression in zebrafish neurons or blood vessels. (FIG. 56A) Brightfield image of embryo 48 hours post-fertilization. (FIG. 56B) Control transgene lacking synthetic CRE fails to drive GFP expression in head of developing zebrafish. (FIG. 56C) Brightfield image of embryo transformed with transgene containing SK-N-SH-specific CRE (N3). (FIG. 56D) GFP channel of FIG. 56C. shows transgene expression in neurons. (FIG. 56E) Brightfield image of embryo transformed with transgene containing SK-N-SH-specific CRE. (FIG. 56F) GFP channel of FIG. 56E shows transgene expression in neurons. (FIG. 56G) Merged FIG. 56E and FIG. 56F (FIG. 56H) Zoom in of FIG. 56D. (FIG. 56I) Brightfield image of embryo transformed with another transgene containing SK-N-SH-specific CRE (N4). (FIG. 56J) N4 drives transgene expression in zebrafish blood vessel. (FIG. 56K) Merged FIG. 56I and FIG. 56J. (FIG. 56L) Zoom in of FIG. 56J. Panels FIG. 56A-FIG. 56D, FIG. 56H: Dorsal views, anterior top. Panels FIG. 56E-FIG. 56G, FIG. 56I-FIG. 56L: Anterior to the left, dorsal top.

FIG. 57A-57H—Additional images from mouse transgenic experiments. (FIG. 57D) Synthetic neuronal CRE #1 and minP drive transgene expression in developing mouse forebrains. Day 14.5 mouse embryos whole animal lacZ staining. No control mouse. (FIG. 57H) Biological replicate of FIG. 57D. (FIG. 57C) Control brains without transgene drive minor transcriptional activation in 5 week old mice. Duplicated from FIG. 21D. (FIG. 57G) Biological replicate of FIG. 57C. (FIG. 57B) Neuronal CRE #1 drives transgene expression cortical layer 6 in 5 week old mouse brains in 3 out of 4 animals. First image is duplicated from FIG. 21D. (FIG. 57F) Biological replicate of panel FIG. 57B. (FIG. 57A) Biological replicate of panel FIG. 57B. (FIG. 57E) Biological replicate of panel FIG. 57B.

FIG. 58A-58B—Immunohistochemistry of N1 CRE activity in the mouse cortex. (FIG. 58A) Representative fluorescence and brightfield images showing expression patterns of neuronal marker, NeuN (top left) and LacZ (top right) across the whole brain. Boxed regions represent the somatosensory cortex(S) and visual cortex (V), digitally zoomed in bottom image; scale bars: 1 mm (top images) and 100 μm (bottom images). Arrows indicate LacZ expression in layer 6. (FIG. 58B) Fluorescence intensity profile plots from quantification of LacZ signal intensity across layers in the somatosensory cortex and visual cortex for non-transgenic control (blue, as represented in greyscale) and N1 CRE transgenic mouse (black).

FIG. 59—Projection of efficiency of zero-order Markov chains for model directed sequence design. 200-mers were uniformly randomly sampled (i.e., sampled from a zero-order Markov chain) and tested using Malinois to calculate MinGap for K562 targeting sequences. Applicant plotted the negative MinGap of the cumulatively best 15000 elements collected over 3000000 steps with 2048 samples taken at each step (total of 6.144 billion elements screened). We plot the median (blue line, as represented in greyscale) and 95%-tile interval (blue shaded region, as represented in greyscale) of the negative MinGap trajectory of the best element collection. As a comparison, we designed 15000 elements using Fast SeqProp (52.1 minutes) and Simulated Annealing (31.5 minutes) with the same objective and plotted the median and 95%-tile intervals of predicted MinGap for these groups.

The figures herein are for illustrative purposes only and are not necessarily drawn to scale.

### DETAILED DESCRIPTION OF THE EXAMPLE EMBODIMENTS

Before the present disclosure is described in greater detail, it is to be understood that this disclosure is not limited to particular embodiments described, and as such may, of course, vary. It is also to be understood that the terminology used herein is for the purpose of describing particular embodiments only and is not intended to be limiting.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are cited to disclose and describe the methods and/or materials in connection with which the publications are cited. All such publications and patents are herein incorporated by references as if each individual publication or patent were specifically and individually indicated to be incorporated by reference. Such incorporation by reference is expressly limited to the methods and/or materials described in the cited publications and patents and does not extend to any lexicographical definitions from the cited publications and patents. Any lexicographical definition in the publications and patents cited that is not also expressly repeated in the instant application should not be treated as such and should not be read as defining any terms appearing in the accompanying claims. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Where a range is expressed, a further aspect includes from the one particular value and/or to the other particular value. Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure. For example, where the stated range includes one or both of the limits, ranges excluding either or both of those included limits are also included in the disclosure, e.g., the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y′, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y′, and ‘greater than z’. In addition, the phrase “about ‘x’ to ‘y’”, where ‘x’ and ‘y’ are numerical values, includes “about ‘x’ to about ‘y’”.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

### General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2nd edition (1989) (Sambrook, Fritsch, and Maniatis); Molecular Cloning: A Laboratory Manual, 4th edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2^{nd} edition 2013 (E. A. Greenfield ed.); Animal Cell Culture (1987) (R. I. Freshney, ed.); Benjamin Lewin, Genes IX, published by Jones and Bartlett, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2^{nd} ed., J. Wiley & Sons (New York, N.Y. 1994), March, Advanced Organic Chemistry Reactions, Mechanisms and Structure 4th ed., John Wiley & Sons (New York, N. Y. 1992); and Marten H. Hofker and Jan van Deursen, Transgenic Mouse Methods and Protocols, 2^{nd} edition (2011).

Definitions of common terms and techniques in chemistry and organic chemistry can be found in Smith. Organic Synthesis, published by Academic Press. 2016; Tinoco et al. Physical Chemistry, 5^{th} edition (2013) published by Pearson; Brown et al., Chemistry, The Central Science 14^{th} ed. (2017), published by Pearson, Clayden et al., Organic Chemistry, 2^{nd} ed. 2012, published by Oxford University Press; Carey and Sunberg, Advanced Organic Chemistry, Part A: Structure and Mechanisms, 5^{th} ed. 2008, published by Springer; Carey and Sunberg, Advanced Organic Chemistry, Part B: Reactions and Synthesis, 5^{th} ed. 2010, published by Springer, and Vollhardt and Schore, Organic Chemistry, Structure and Function; 8^{th} ed. (2018) published by W.H. Freeman.

Definitions of common terms, analysis, and techniques in genetics can be found in e.g., Hartl and Clark. Principles of Population Genetics. 4^{th} Ed. 2006, published by Oxford University Press. Published by Booker. Genetics: Analysis and Principles, 7^{th} Ed. 2021, published by McGraw Hill; Isik et al., Genetic Data Analysis for Plant and Animal Breeding. First ed. 2017. published by Springer International Publishing AG; Green, E. L. Genetics and Probability in Animal Breeding Experiments. 2014, published by Palgrave; Bourdon, R. M. Understanding Animal Breeding. 2000 2^{nd} Ed. published by Prentice Hall; Pal and Chakravarty. Genetics and Breeding for Disease Resistance of Livestock. First Ed. 2019, published by Academic Press; Fasso, D. Classification of Genetic Variance in Animals. First Ed. 2015, published by Callisto Reference; Megahed, M. Handbook of Animal Breeding and Genetics, 2013, published by Omniscriptum Gmbh & Co. Kg., LAP Lambert Academic Publishing; Reece. Analysis of Genes and Genomes. 2004, published by John Wiley & Sons. Inc; Deonier et al., Computational Genome Analysis. 5^{th} Ed. 2005, published by Springer-Verlag, New York; Meneely, P. Genetic Analysis: Genes, Genomes, and Networks in Eukaryotes. 3^{rd} Ed. 2020, published by Oxford University Press.

As used herein, the singular forms “a”, “an”, and “the” include both singular and plural referents unless the context clearly dictates otherwise.

As used herein, “about,” “approximately,” “substantially,” and the like, when used in connection with a measurable variable such as a parameter, an amount, a temporal duration, and the like, are meant to encompass variations of and from the specified value including those within experimental error (which can be determined by e.g. given data set, art accepted standard, and/or with e.g. a given confidence interval (e.g. 90%, 95%, or more confidence interval from the mean), such as variations of +/−10% or less, +/−5% or less, +/−1% or less, and +/−0.1% or less of and from the specified value, insofar such variations are appropriate to perform in the disclosed invention. As used herein, the terms “about,” “approximate,” “at or about,” and “substantially” can mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

As used herein, a “biological sample” refers to a sample obtained from, made by, secreted by, excreted by, or otherwise containing part of or from a biologic entity. A biologic sample can contain whole cells and/or live cells and/or cell debris, and/or cell products, and/or virus particles. The biological sample can contain (or be derived from) a “bodily fluid”. The biological sample can be obtained from an environment (e.g., water source, soil, air, and the like). Such samples are also referred to herein as environmental samples. As used herein “bodily fluid” refers to any non-solid excretion, secretion, or other fluid present in an organism and includes, without limitation unless otherwise specified or is apparent from the description herein, amniotic fluid, aqueous humor, vitreous humor, bile, blood or component thereof (e.g. plasma, serum, etc.), breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from an organism, for example by puncture, or other collecting or sampling procedures.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

As used herein, “identity,” refers to a relationship between two or more nucleotide or polypeptide sequences, as determined by comparing the sequences. In the art, “identity” also refers to the degree of sequence relatedness between polynucleotide or polypeptide sequences as determined by the match between strings of such sequences. “Identity” can be readily calculated by known methods, including, but not limited to, those described in (Computational Molecular Biology, Lesk, A. M., Ed., Oxford University Press, New York, 1988; Biocomputing: Informatics and Genome Projects, Smith, D. W., Ed., Academic Press, New York, 1993; Computer Analysis of Sequence Data, Part I, Griffin, A. M., and Griffin, H. G., Eds., Humana Press, New Jersey, 1994; Sequence Analysis in Molecular Biology, von Heinje, G., Academic Press, 1987; and Sequence Analysis Primer, Gribskov, M. and Devereux, J., Eds., M Stockton Press, New York, 1991; and Carillo, H., and Lipman, D., SIAM J. Applied Math. 1988, 48:1073. Preferred methods to determine identity are designed to give the largest match between the sequences tested. Methods to determine identity are codified in publicly available computer programs. The percent identity between two sequences can be determined by using analysis software (e.g., Sequence Analysis Software Package of the Genetics Computer Group, Madison Wis.) that incorporates the Needelman and Wunsch, (J. Mol. Biol., 1970, 48:443-453,) algorithm (e.g., NBLAST, and XBLAST). The default parameters are used to determine the identity for the polypeptides or polynucleotides of the present disclosure, unless stated otherwise.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

All publications, published patent documents, and patent applications cited herein are hereby incorporated by reference to the same extent as though each individual publication, published patent document, or patent application was specifically and individually indicated as being incorporated by reference.

### Overview

Gene regulation is fundamental to the identity and survival of every cell. While less than 2% of the human genome is dedicated to protein-coding sequence, at least 19% of the genome is associated with open chromatin or transcription factor binding. However, despite their prevalence in the genome, relatively few cis-regulatory elements (CREs) have been directly shown to regulate a target gene. Progress towards comprehensive characterization of CREs has potential to decode the DNA sequence-dependent rules underpinning gene regulation. Consolidating these rules into a regulatory grammar can reveal how CRE-gene interaction networks govern normal development and cell biology.

Genetic variants in CREs contribute to phenotypic diversity both within and between species. Therefore, accurate modeling of the regulatory grammar of the genome would revolutionize the interpretation of genetic variants impacting adaptive evolution and disease. Massively parallel reporter assays (MPRA) are an orthogonal technology enabling rapid, direct characterization of hundreds of thousands of CREs and the genetic variants within them. However, MPRA lacks the throughput for dense genome-wide characterization.

In several exemplary embodiments herein, Applicant describes a deep learning model of cis-regulatory activity for discovery of enhancer function, characterization of human variation, and engineering of synthetic CREs. Without being bound by theory, Applicant demonstrates that deep learning models trained on MPRA data can accurately extrapolate CRE function genome-wide. Furthermore, not only can these models accurately predict the consequence of genetic variation on CRE function, Applicant also successfully deployed them to engineer artificial CREs ab initio. Further, the methods and techniques described herein can support elucidation of CRE syntax in the genome. Illuminating the role of non-coding variation in evolution and health will unlock new, highly targeted approaches in medicine.

The embodiments disclosed herein can utilize machine learning to identify or design cis-regulatory elements with cell-type, cell state, tissue type, and/or environment specific activity, as further defined below, which in turn allows for the design and generation of synthetic non-naturally occurring cell type-specific regulatory elements.

Typically, empirical reporter assays, such as massively parallel reporter assays (MPRAs), are required to directly characterize cis-regulatory function of DNA sequences. These methods need to have the sensitivity necessary to accurately measure the impacts of genetic variants. These methods are time-consuming and even more so when used on genomes or iteratively used on modified sequences. In many instances, the sample space for engineered sequences is limited because of the impossible about of time needed.

Conventional systems are not configured to identify or design cis-regulatory elements with cell-type specific activity rapidly and over a large sample space. Typically, conventional systems cannot access real-time infrastructure data when a user is suffering from a pain point. Conventional systems do not facilitate real-time identification or design cis-regulatory elements with cell-type specific activity. The systems do not provide solutions in a manner that is quick and painless for users. Conventional systems are not able to identify or design cis-regulatory elements with cell-type specific activity in real-time from one or more nucleic acid sequences.

Further, conventional methods identify cis-regulatory elements with cell-type specific activity based on human assessments of time consuming empirical reporter assays. Human systems are unable to identify or design cis-regulatory elements with cell-type specific activity from one or more nucleic acid sequences in real time. Unlike a machine learning system or artificial intelligence system, humans are unable to draw the subtle conclusions required to identify or design cis-regulatory elements with cell-type specific activity from one or more nucleic acid sequences. Human systems are unable to create predictive models based on combined data collected from, for example, a suitable database, such as CREs centered on variants from the UK Biobank and/or GTEx.

In one aspect, technologies herein provide methods to use machine learning systems to identify or design cis-regulatory elements with cell-type, cell state, tissue type, and/or environment specific activity from one or more nucleic acid sequences. The machine learning systems uses CRE-activity data set obtained from a suitable database to create models that can predict CRE-activity. Because of the immense amount of data that is acquired, processed, and categorized, any number of human users would be unable to create the predictive models or perform the operations described herein.

This invention represents an advance in computer engineering that represents a substantial advancement over existing practices. The data acquired to prepare the predictive models are technical data relating to CRE-activity data. The outputs of the machine learning systems are not obtainable by humans or by conventional methods. Identifying CRE activity from a one or more nucleic acid sequence creates a predictive system that is a non-conventional, technical, real-world output and benefit that is not obtainable with conventional systems. The methods and systems described herein are more consistent, accurate, and efficient than manual/human analysis, which is prone to bias and doesn't scale to the amount of qualitative data that is generated today.

Standard techniques related to making and using aspects of the invention may or may not be described in detail herein. Various aspects of computing systems and specific computer programs to implement the various technical features described herein are well known.

Other compositions, compounds, methods, features, and advantages of the present disclosure will be or become apparent to one having ordinary skill in the art upon examination of the following drawings, detailed description, and examples. It is intended that all such additional compositions, compounds, methods, features, and advantages be included within this description, and be within the scope of the present disclosure.

### Generating Cis-Regulatory Elements

### Example System Architectures

Turning now to the drawings, in which like numerals represent like (but not necessarily identical) elements throughout the figures, example embodiments are described in detail.

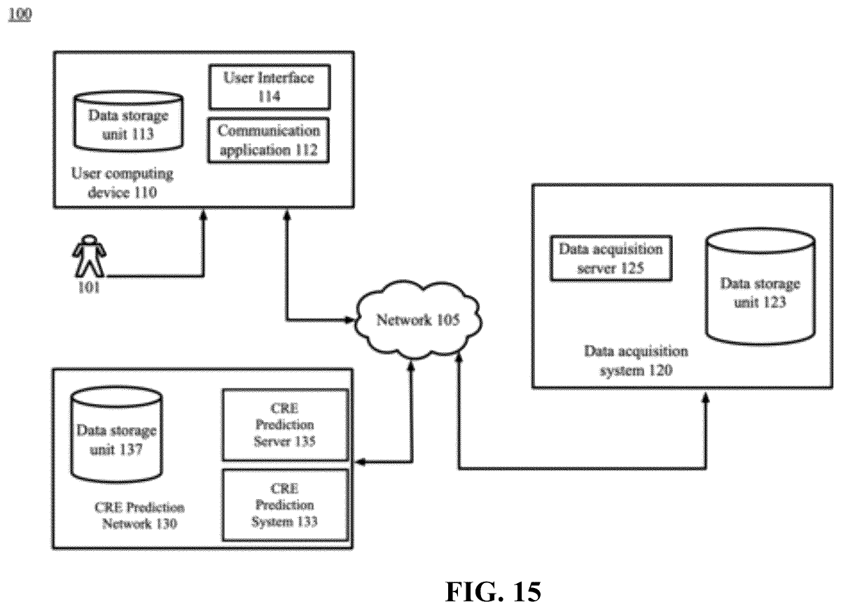

FIG. 15 is a block diagram depicting a system 100 to identify or design cis-regulatory elements with cell-type, cell state, tissue type, and/or environment specific activity and perform machine learning on one or more nucleic acid sequences. In one example embodiment, a user 101 associated with a user computing device 110 must install an application, and or make a feature selection to obtain the benefits of the techniques described herein.

As depicted in FIG. 15, the system 100 includes network computing devices/systems 110, 120, and 130 that are configured to communicate with one another via one or more networks 105 or via any suitable communication technology.

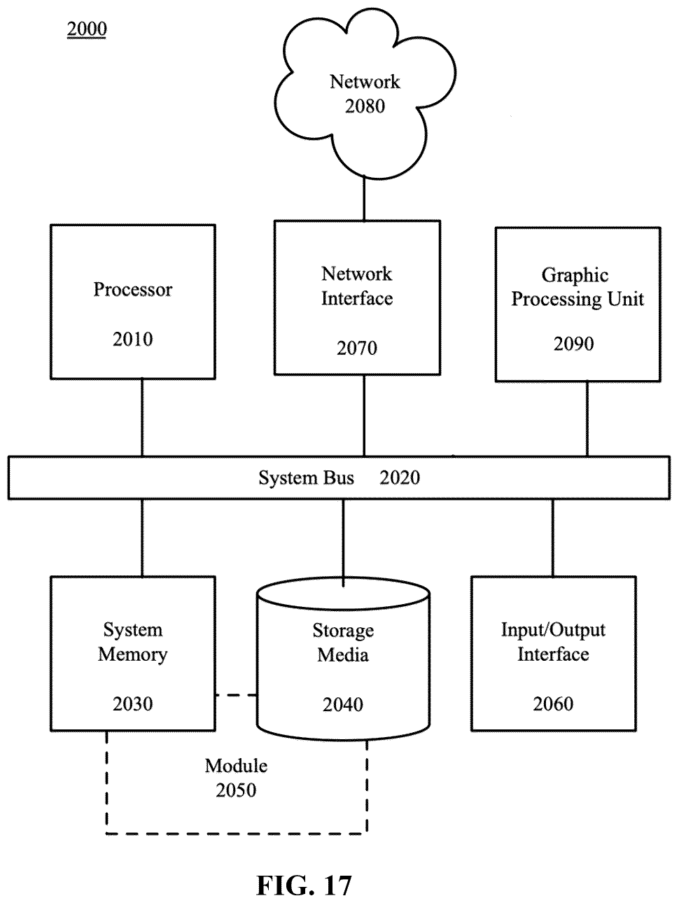

Each network 105 includes a wired or wireless telecommunication means by which network devices/systems (including devices 110, 120, and 130) can exchange data. For example, each network 105 can include any of those described herein such as the network 2080 described in FIG. 17 or any combination thereof or any other appropriate architecture or system that facilitates the communication of signals and data. Throughout the discussion of example embodiments, it should be understood that the terms “data” and “information” are used interchangeably herein to refer to text, images, audio, video, or any other form of information that can exist in a computer-based environment. The communication technology utilized by the devices/systems 110, 120, and 130 may be similar networks to network 105 or an alternative communication technology.

Each network computing device/system 110, 120, and 130 includes a computing device having a communication module capable of transmitting and receiving data over the network 105 or a similar network. For example, each network device/system 110, 120, and 130 can include any computing machine 2000 described herein and found in FIG. 17 or any other wired or wireless, processor-driven device. In the example embodiment depicted in FIG. 15, the network devices/systems 110, 120, and 130 are operated by user 101, data acquisition system operators, and CRE prediction operators, respectively.

The user computing device 110 includes a user interface 114. The user interface 114 may be used to display a graphical user interface and other information to the user 101 to allow the user 101 to interact with the data acquisition system 120, the CRE prediction system 130, and others. The user interface 114 receives user input for data acquisition and/or machine learning and displays results to user 101. In another example embodiment, the user interface 114 may be provided with a graphical user interface by the data acquisition system 120 and or the CRE prediction system 130. The user interface 114 may be accessed by the processor of the user computing device 110. The user interface may display 114 may display a webpage associate with the data acquisition system 120 and/or the CRE prediction system 130. The user interface 114 may be used to provide input, configuration data, and other display direction by the webpage of the data acquisition system 120 and/or the CRE prediction system 130. In another example embodiment, the user interface 114 may be managed by the data acquisition system 120, the CRE prediction system 130, or others. In another example embodiment, the user interface 114 may be managed by the user computing device 110 and be prepared and displayed to the user 101 based on the operations of the user computing device 110.

The user 101 can use the communication application 112 on the user computing device 110, which may be, for example, a web browser application or a stand-alone application, to view, download, upload, or otherwise access documents or web pages through the user interface 114 via the network 105. The user computing device 110 can interact with the web servers or other computing devices connected to the network, including the data acquisition server 125 of the data acquisition system 120 and the CRE prediction server 135 of the CRE prediction system 130. In another example embodiment, the user computing device 110 communicates with devices in the data acquisition system 120 and/or the CRE prediction system 130 via any other suitable technology, including the example computing system described below.

The user computing device 110 also includes a data storage unit 113 accessible by the user interface 114, the communication application 112, or other applications. The example data storage unit 113 can include one or more tangible computer-readable storage devices. The data storage unit 113 can be stored on the user computing device 110 or can be logically coupled to the user computing device 110. For example, the data storage unit 113 can include on-board flash memory and/or one or more removable memory accounts or removable flash memory. In another example embodiments, the data storage unit 113 may reside in a cloud-based computing system.

An example data acquisition system 120 comprises a data storage unit 123 and an acquisition server 125. The data storage unit 123 can include any local or remote data storage structure accessible to the data acquisition system 120 suitable for storing information. The data storage unit 123 can include one or more tangible computer-readable storage devices, or the data storage unit 123 may be a separate system, such as a different physical or virtual machine or a cloud-based storage service.

In one aspect, the data acquisition server 125 communicates with the user computing device 110 and/or the CRE prediction system 130 to transmit requested data. The data may include one or more nucleic acid sequences or predicted CRE activity.

An example CRE prediction system 130 comprises a machine learning system 133, a CRE prediction server 135, and a data storage unit 137. The CRE prediction server 135 communicates with the user computing device 110 and/or the data acquisition system 120 to request and receive data. The data may comprise the data types previously described in reference to the data acquisition server 125.

The CRE prediction system 133 receives an input of data from the CRE prediction server 135. The CRE prediction system 133 can comprise one or more functions to implement any of the mentioned training methods to learn a CRE activity of one or more nucleic acid sequences. In a preferred embodiment, the machine learning program may comprise a convolutional neural network. Any suitable architecture may be applied to learn the complex pattern of sequences that interact with transcription factors to control gene expression.

The data storage unit 137 can include any local or remote data storage structure accessible to the CRE prediction system 130 suitable for storing information. The data storage unit 137 can include one or more tangible computer-readable storage devices, or the data storage unit 137 may be a separate system, such as a different physical or virtual machine or a cloud-based storage service.

In an alternate embodiment, the functions of either or both of the data acquisition system 120 and the CRE prediction system 130 may be performed by the user computing device 110.

It will be appreciated that the network connections shown are examples, and other means of establishing a communications link between the computers and devices can be used. Moreover, those having ordinary skill in the art having the benefit of the present disclosure will appreciate that the user computing device 110, data acquisition system 120, and the CRE prediction system 130 illustrated in FIG. 15 can have any of several other suitable computer system configurations. For example, a user computing device 110 embodied as a mobile phone or handheld computer may not include all the components described above.

In example embodiments, the network computing devices and any other computing machines associated with the technology presented herein may be any type of computing machine such as, but not limited to, those discussed in more detail with respect to FIG. 17. Furthermore, any modules associated with any of these computing machines, such as modules described herein or any other modules (scripts, web content, software, firmware, or hardware) associated with the technology presented herein may by any of the modules discussed in more detail with respect to FIG. 17. The computing machines discussed herein may communicate with one another as well as other computer machines or communication systems over one or more networks, such as network 105. The network 105 may include any type of data or communications network, including any of the network technology discussed with respect to FIG. 17.

### Example Processes

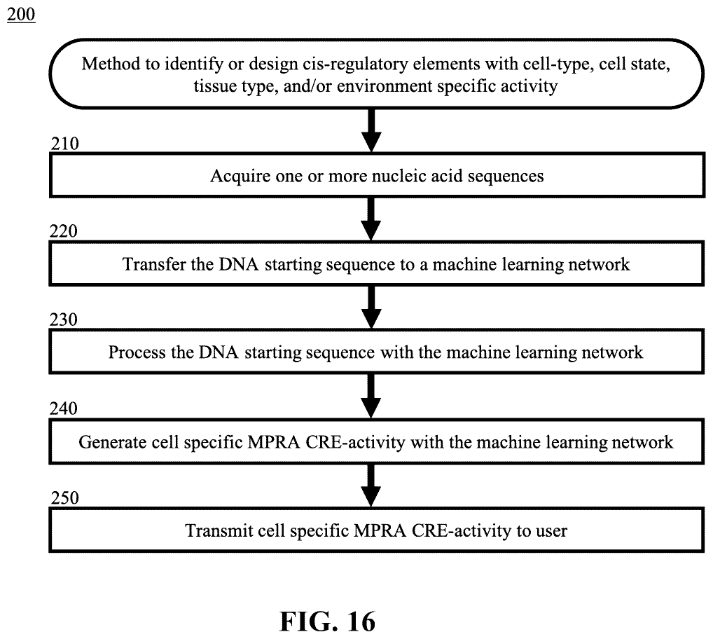

The example methods illustrated in FIG. 16 is described hereinafter with respect to the components of the example architecture 100. The example methods also can be performed with other systems and in other architectures including similar elements.

Referring to FIG. 16, and continuing to refer to FIG. 15 for context, a block flow diagram illustrates methods 200 to identify or design cis-regulatory elements with cell-type, cell state, tissue type, and/or environment specific activity, in accordance with certain examples of the technology disclosed herein.

In block 210, the CRE prediction system 130 receives an input of one or more nucleic acid sequences. The CRE prediction system 130 may receive the one or more nucleic acid sequences from the user computing device 110, the data acquisition system 120, or any other suitable source of the one or more nucleic acid sequences via the network 105 to the CRE prediction system 130, discussed in more detail in other sections herein. The acquisition engine comprises any software or hardware individually or in combination described herein that is capable of communicating with a user device, such as fetching, receiving, or sending information, thereby allowing access to the one or more nucleic acid sequences or predict CRE activity by the CRE prediction system 130 or the data acquisition system 120.

### Sequence Generation Algorithms

In example, embodiments, the initial one or more nucleic acid sequences for the first iteration is a nucleic acid sequence generated from any suitable nucleic acid sequence generation algorithms. Typically, a nucleic acid sequence generation algorithm will generate a nucleic acid sequence of a designated length and nucleotide percentage. Generated nucleic acid sequences may have a nucleotide distribution similar to that of exonic, intronic, or intergenic sequences. In example embodiments, the nucleotide distribution is generated at random. Nucleic acid sequence generation algorithms are well known in the art and briefly described herein. See e.g., Piva F, Principato G. RANDNA: a random DNA sequence generator. In Silico Biol. 2006; 6 (3): 253-8 incorporated herein by reference.

In example embodiments, the sequence generation algorithms is AdaLead, FastSeqProp, simulated annealing, or gradient based updates with random momentum (GRUM).

AdaLead is an evolutionary greedy algorithm, which uses an iterative approach wherein a set of seed sequences are recombined and mutated. Any new sequence meeting a designated threshold is added to the original set. The highest ranking sequences from the set are used for the next iteration. See e.g., Sinai, Sam, et al. “AdaLead: A simple and robust adaptive greedy search algorithm for sequence design.” arXiv preprint arXiv: 2010.02141 (2020) incorporated herein by reference.

Fast SeqProp is a modified activation maximization method, which combines a logit normalization scheme with a softmax straight-through estimator. The method begins with a randomly initialized logit matrix, which is optimized with a discrete nucleotide sampler using scaled, normalized logits ((scaled) as parameters. The gradients are formed using a softmax ST estimator. See e.g., Linder, Johannes, and Georg Seelig. “Fast activation maximization for molecular sequence design.” BMC bioinformatics 22 (2021): 1-20 incorporated herein by reference.

Simulated Annealing (SA) attempts to describe and predict particle rearrangement through a thermal heat bath cycle. SA uses the Metropolis algorithm (MA) to determine whether a given configuration is acceptable at a given thermal state. The MA may also be used to generate sequences of a combinatorial optimization problem. Given an engineered sequence comprising one or more mutations, the MA algorithm can describe and predict the thermal perturbation caused by the one or more mutations. See e.g., Van Laarhoven, Peter J M, et al. Simulated annealing. Springer Netherlands, 1987. incorporated herein by reference.

Gradient-based Updates with Random Momentum (GRUM) uses an un-normalized probability distribution wherein backpropagation to the inputs is enabled by reparameterizing discrete nucleotide sequences using the Gumbel-Softmax trick (i.e., a method to draw sample from a categorical distribution with class probabilities; See e.g., Jang, E., Gu, S., & Poole, B. (2017). Categorical Reparametrization with Gumble-Softmax. In ICLR 2017-Conference Track. Amherst, MA). The reparametrized inputs were then sampled using the No-U-Turn Sampler (i.e., a modified Hamiltonian Monte Carlo (HMC) algorithm; See e.g., Hoffman, Matthew D., and Andrew Gelman. “The No-U-Turn sampler: adaptively setting path lengths in Hamiltonian Monte Carlo.” J. Mach. Learn. Res. 15.1 (2014): 1593-1623. Finally, the discrete DNA sequences were sampled.

In block 220, the one or more nucleic acid sequences is transferred over a network via the transfer engine from the user associated device 100 or the data acquisition system 120 to the CRE prediction system 130. The transfer engine comprises any software or hardware individually or in combination described herein that is capable of moving or transferring the one or more nucleic acid sequences thereby allowing access within the CRE prediction system 130.

In block 230, the CRE prediction system 130 receives input of the one or more nucleic acid sequences and passes the one or more nucleic acid sequences to the CRE prediction server 135 wherein the cis-regulatory elements with cell-type specific activity are identified or designed. The CRE prediction system 133 processes the data of the one or more nucleic acid sequences into output data comprising information containing CRE activity. In example embodiments, the one or more nucleic acid sequences is processed with one or more of the machine learning methods described herein.

Because the design of one or more cell-specific engineered cis-regulatory elements is performed by the machine learning algorithm based on data collected by the data acquisition system 120, human analysis or cataloging is not required. The process is performed automatically by the machine learning system 130 without human intervention, as described in the machine learning section below. The amount of data typically collected includes thousands to tens of thousands of data items for each one or more nucleic acid sequences and CRE-activity. The one or more nucleic acid sequences may include is a genome or a portion thereof, an epigenome or portion thereof, or a nucleic acid sequence generated from a suitable DNA sequence generation algorithm. (e.g., evolutionary, probabilistic, simulated annealing, or gradient based updates with random momentum (GRUM)). Human intervention in the process is not useful or required because the amount of data is too great. A team of humans would not be able to catalog or analyze the data in any useful manner. Moreover, a human cannot obtain one or more nucleic acid sequences and from that data identify cis-regulatory elements with cell-type specific activity.

In block 240, the machine learning output is generated. Within the CRE prediction system 133, the output data from the machine learning system is processed into user comprehensible information comprising CRE activity. In example embodiments, the CRE activity is cell type, cell state, tissue type, or environment specific MPRA CRE-activity. Cell type specific CRE activity may refer to one or more cells that share one or more morphological or phenotypical features that have CRE activity. Cell state specific CRE activity may refer to one or more cell types in a particular reference frame (i.e., time frame) that have CRE activity.

Tissue type specific CRE activity may refer to any of the four types of tissue: connective, epithelial, muscle, or nervous that have CRE activity. In particular, connective tissue may refer to tissue that supports other tissues and binds them together (e.g., bone, blood, and lymph tissues), epithelial tissue may refer to tissue that provides a protective layer (e.g., skin, the linings of internal passages), muscle tissue may refer to striated (i.e., voluntary) muscles (e.g., muscle that moves the skeleton) and/or smooth muscle (e.g., muscles that surround the stomach), nervous tissue is made up of nerve cells (i.e., neurons). Environment specific MPRA CRE-activity may refer to cells cultured under particularly conditions that have CRE activity. In particular, environment specific MPRA CRE-activity may refer to an MPRA assay (or any other similar reporter assay) that is performed with cells under the influence of a particular environmental condition (e.g. a thermal insult, energy insult, radiation, pH insult, osmolarity insult, strain, pressure, etc.) such that the CREs that are identified as active are unique to those particular environmental conditions.

### Objective Function

In example embodiments, wherein processing further comprises passing the prediction to a cell, tissue, or environment specific regulatory optimizing objective function that maximizes cell specific regulatory activity. Generally, an objective function represents a linear optimization problem, for example see the Linear Regression section described herein. The optimization problem refers to any problem seeking a maximized or minimized solution, for example, maximizing predicted expression of a given sequence in one cell type while reducing expression in the other cells. Objective functions are well known in the art and examples of objective functions are further described here. In example embodiments, the objective function is specific for promoter activity, enhancer activity, silencer activity, or insulator activity of cell type, cell state, tissue type, or environment specific regulatory activity. In example embodiments, the objective function maximizes the predicted expression of a given sequence in one cell type, cell state, tissue type, or environment while reducing expression in all other cell types, cell states, tissue types, or environments. In example embodiments, the objective function prioritizes nucleic acid sequences with cell type, cell state, tissue type, or environment specific promoter activity, enhancer activity, silencer activity, or insulator activity.

In example embodiments, processing further comprises iterative cell, tissue, or environment specific regulatory optimization of the one or more nucleic acid sequence, wherein iterative cell, tissue, or environment specific regulatory optimization comprises sequentially modifying the nucleic acid sequence in each iteration. Iterative cell specific regulatory optimization may comprise the steps of a) passing one or more nucleic acid sequence to the machine learning network b) receiving the CRE-activity prediction output c) separating from the one or more nucleic acid sequences, any one or more nucleic acid sequences that are not predicted to have CRE-activity (the remaining set may also be referred to as the new set or iterative set) d) modifying (e.g., substituting, removing, or adding) one or more (e.g., 1, 2, 3, 4, 5, 6, 7, 8, 9, 10, . . . , 100 or any range therein) nucleic acids in the one or more nucleic acid sequences and e) repeating steps (a)-(d) until the remaining one or more nucleic acid sequences have reached a designated threshold for CRE-activity.

In example embodiments, the process further comprises updating the one or more nucleic acid sequences in each iteration based on the output of the cell, tissue, or environment specific regulatory optimizing objective function. In example embodiments, in between steps (c) and (d), the remaining one or more nucleic acid sequences are passed to an objective function as described herein. Similar to step c) above, any of the remaining one or more nucleic acid sequences that do not do not return a maximized value at or above a designated threshold are separated from the remaining one or more nucleic acid sequences. The new remaining one or more nucleic acid sequences are then modified as described in step (d) above.

In block 250, the CRE activity is transmitted back to the user via the network 105. In example embodiments, the resulting user information is stored on the data storage unit 137. In example embodiments, the resulting user information is immediately transmitted to the user's device. In example embodiments, the resulting user information is transmitted across the network 105 to the data acquisition system for subsequent access by the user associated device 100 or CRE prediction system 130.

The ladder diagrams, scenarios, flowcharts and block diagrams in the figures and discussed herein illustrate architecture, functionality, and operation of example embodiments and various aspects of systems, methods, and computer program products of the present invention. Each block in the flowchart or block diagrams can represent the processing of information and/or transmission of information corresponding to circuitry that can be configured to execute the logical functions of the present techniques. Each block in the flowchart or block diagrams can represent a module, segment, or portion of one or more executable instructions for implementing the specified operation or step. In example embodiments, the functions/acts in a block can occur out of the order shown in the figures and nothing requires that the operations be performed in the order illustrated. For example, two blocks shown in succession can executed concurrently or essentially concurrently. In another example, blocks can be executed in the reverse order. Furthermore, variations, modifications, substitutions, additions, or reduction in blocks and/or functions may be used with any of the ladder diagrams, scenarios, flow charts and block diagrams discussed herein, all of which are explicitly contemplated herein.

The ladder diagrams, scenarios, flow charts and block diagrams may be combined with one another, in part or in whole. Coordination will depend upon the required functionality. Each block of the block diagrams and/or flowchart illustration as well as combinations of blocks in the block diagrams and/or flowchart illustrations can be implemented by special purpose hardware-based systems that perform the aforementioned functions/acts or carry out combinations of special purpose hardware and computer instructions. Moreover, a block may represent one or more information transmissions and may correspond to information transmissions among software and/or hardware modules in the same physical device and/or hardware modules in different physical devices.

The present techniques can be implemented as a system, a method, a computer program product, digital electronic circuitry, and/or in computer hardware, firmware, software, or in combinations of them. The system may comprise distinct software modules embodied on a computer readable storage medium; the modules can include, for example, any or all of the appropriate elements depicted in the block diagrams and/or described herein; by way of example and not limitation, any one, some or all of the modules/blocks and or sub-modules/sub-blocks described. The method steps can then be carried out using the distinct software modules and/or sub-modules of the system, as described above, executing on one or more hardware processors such as a CPU or GPU.

The computer program product can include a program tangibly embodied in an information carrier (e.g., computer readable storage medium or media) having computer readable program instructions thereon for execution by, or to control the operation of, data processing apparatus (e.g., a processor) to carry out aspects of one or more embodiments of the present invention. It will be understood that each block of the flowchart illustrations and/or block diagrams, and combinations of blocks in the flowchart illustrations and/or block diagrams, can be implemented by computer readable program instructions.

The computer readable program instructions can be performed on general purpose computing device, special purpose computing device, or other programmable data processing apparatus to produce a machine, such that the instructions, which execute via, at least partially, by one or more processors that are temporarily configured (e.g., by software) or permanently configured to perform the functions/acts specified in the flowchart and/or block diagram block or blocks. The processors, either: temporarily or permanently; or partially configured, may comprise processor-implemented modules. The present techniques referred to herein may, in example embodiments, comprise processor-implemented modules. Functions/acts of the processor-implemented modules may be distributed among the one or more processors. Moreover, the functions/acts of the processor-implements modules may be deployed across a number of machines, where the machines may be located in a single geographical location or distributed across a number of geographical locations.

The computer readable program instructions can also be stored in a computer readable storage medium that can direct one or more computer devices, programmable data processing apparatuses, and/or other devices to carry out the function/acts of the processor-implemented modules. The computer readable storage medium containing all or partial processor-implemented modules stored therein, comprises an article of manufacture including instructions which implement aspects, operations, or steps to be performed of the function/act specified in the flowchart and/or block diagram block or blocks.

Computer readable program instructions described herein can be downloaded to a computer readable storage medium within a respective computing/processing devices from a computer readable storage medium. Optionally, the computer readable program instructions can be downloaded to an external computer device or external storage device via a network. A network adapter card or network interface in each computing/processing device can receive computer readable program instructions from the network and forward the computer readable program instructions for permanent or temporary storage in a computer readable storage medium within the respective computing/processing device.

Computer readable program instructions described herein can be assembler instructions, instruction-set-architecture (ISA) instructions, machine instructions, machine dependent instructions, microcode, firmware instructions, state-setting data, or either source code or object code. The computer readable program instructions can be written in any programming language such as compiled or interpreted languages. In addition, the programming language can be object-oriented programming language (e.g. “C++”) or conventional procedural programming languages (e.g. “C”) or any combination thereof may be used to as computer readable program instructions. The computer readable program instructions can be distributed in any form, for example as a stand-alone program, module, subroutine, or other unit suitable for use in a computing environment. The computer readable program instructions can execute entirely on one computer or on multiple computers at one site or across multiple sites connected by a communication network, for example on user's computer, partly on the user's computer, as a stand-alone software package, partly on the user's computer and partly on a remote computer or entirely on a remote computer or server. If the computer readable program instructions are executed entirely remote, then the remote computer can be connected to the user's computer through any type of network or the connection can be made to an external computer. In examples embodiments, electronic circuitry including, but not limited to, programmable logic circuitry, field-programmable gate arrays (FPGA), or programmable logic arrays (PLA) can execute the computer readable program instructions. Electronic circuitry can utilize state information of the computer readable program instructions to personalize the electronic circuitry, to execute functions/acts of one or more embodiments of the present invention.

Example embodiments described herein include logic or a number of components, modules, or mechanisms. Modules may comprise either software modules or hardware-implemented modules. A software module may be code embodied on a non-transitory machine-readable medium or in a transmission signal. A hardware-implemented module is a tangible unit capable of performing certain operations and may be configured or arranged in a certain manner. In example embodiments, one or more computer systems (e.g., a standalone, client or server computer system) or one or more processors may be configured by software (e.g., an application or application portion) as a hardware-implemented module that operates to perform certain operations as described herein.

In example embodiments, a hardware-implemented module may be implemented mechanically or electronically. In example embodiments, hardware-implemented modules may comprise permanently configured dedicated circuitry or logic to execute certain functions/acts such as a special-purpose processor or logic circuitry (e.g., a field programmable gate array (FPGA) or an application-specific integrated circuit (ASIC)). In example embodiments, hardware-implemented modules may comprise temporary programmable logic or circuitry to perform certain functions/acts. For example, a general-purpose processor or other programmable processor.

The term “hardware-implemented module” encompasses a tangible entity. A tangible entity may be physically constructed, permanently configured, or temporarily or transitorily configured to operate in a certain manner and/or to perform certain functions/acts described herein. Hardware-implemented modules that are temporarily configured need not be configured or instantiated at any one time. For example, if the hardware-implemented modules comprise a general-purpose processor configured using software, then the general-purpose processor may be configured as different hardware-implemented modules at different times.

Hardware-implemented modules can provide, receive, and/or exchange information from/with other hardware-implemented modules. The hardware-implemented modules herein may be communicatively coupled. Multiple hardware-implemented modules operating concurrently, may communicate through signal transmission, for instance appropriate circuits and buses that connect the hardware-implemented modules. Multiple hardware-implemented modules configured or instantiated at different times may communicate through temporarily or permanently archived information, for instance the storage and retrieval of information in memory structures to which the multiple hardware-implemented modules have access. For example, one hardware-implemented module may perform an operation, and store the output of that operation in a memory device to which it is communicatively coupled. Consequently, another hardware-implemented module may, at some time later, access the memory device to retrieve and process the stored information. Hardware-implemented modules may also initiate communications with input or output devices, and can operate on information from the input or output devices.

In example embodiments, the present techniques can be at least partially implemented in a cloud or virtual machine environment.

### Machine Learning

Machine learning is a field of study within artificial intelligence that allows computers to learn functional relationships between inputs and outputs without being explicitly programmed. Machine learning involves a module comprising algorithms that may learn from existing data by analyzing, categorizing, or identifying the data. Such machine-learning algorithms operate by first constructing a model from training data to make predictions or decisions expressed as outputs. In example embodiments, the training data includes data for one or more identified features and one or more outcomes, for example one or more nucleic acid sequences and CRE-activity, respectively. Although example embodiments are presented with respect to a few machine-learning algorithms, the principles presented herein may be applied to other machine-learning algorithms.

Data supplied to a machine learning algorithm can be considered a feature, which can be described as an individual measurable property of a phenomenon being observed. The concept of feature is related to that of an independent variable used in statistical techniques such as those used in linear regression. The performance of a machine learning algorithm in pattern recognition, classification and regression is highly dependent on choosing informative, discriminating, and independent features. Features may comprise numerical data, categorical data, time-series data, strings, graphs, or images. Features of the invention may further comprise one or more nucleic acid sequences. These one or more nucleic acid sequences may include genome or a portion thereof, an epigenome or portion thereof, or a nucleic acid sequence generated from a suitable nucleic sequence generation algorithm.

In general, there are two categories of machine learning problems: classification problems and regression problems. Classification problems, also referred to as categorization problems, aim at classifying items into discrete category values. Training data teaches the classifying algorithm how to classify. In example embodiments, features to be categorized may include one or more nucleic acid sequences, which can be provided to the classifying machine learning algorithm and then placed into categories of, for example, CRE activity. Regression algorithms aim at quantifying and correlating one or more features. Training data teaches the regression algorithm how to correlate the one or more features into a quantifiable value. In example embodiments, features such as one or more nucleic acid sequences can be provided to the regression machine learning algorithm resulting in one or more continuous values, for example CRE activity.

### Embedding

In one example, the machine learning module may use embedding to provide a lower dimensional representation, such as a vector, of features to organize them based off respective similarities. In some situations, these vectors can become massive. In the case of massive vectors, particular values may become very sparse among a large number of values (e.g., a single instance of a value among 50,000 values). Because such vectors are difficult to work with, reducing the size of the vectors, in some instances, is necessary. A machine learning module can learn the embeddings along with the model parameters. In example embodiments, features such as one or more nucleic acid sequences can be mapped to vectors implemented in embedding methods. In example embodiments, embedded semantic meanings are utilized. Embedded semantic meanings are values of respective similarity. For example, the distance between two vectors, in vector space, may imply two values located elsewhere with the same distance are categorically similar. Embedded semantic meanings can be used with similarity analysis to rapidly return similar values. In example embodiments, one or more nucleic acid sequences is embedded. For example, the one or more nucleic acid sequences are reduced to a vector or matrix that represents the length and nucleic acid identity of the one or more nucleic acid sequences. In example embodiments, the methods herein are developed to identify meaningful portions of the vector and extract semantic meanings between that space.

### Training Methods

In example embodiments, the machine learning module can be trained using techniques such as unsupervised, supervised, semi-supervised, reinforcement learning, transfer learning, incremental learning, curriculum learning techniques, and/or learning to learn. Training typically occurs after selection and development of a machine learning module and before the machine learning module is operably in use. In one aspect, the training data used to teach the machine learning module can comprise input data such as one or more nucleic acid sequences (e.g., massively parallel reporter assays (MPRA) data) and the respective target output data such as CRE activity.

### CRE-Activity Database

In example embodiments, the machine learning network is trained on nucleic acid sequences and their corresponding CRE-activity. In example embodiments, the nucleic acid sequences and optionally the CRE-activity are derived from a suitable database. A suitable database comprises nucleic acid sequences, such as a genomic database and optionally the corresponding CRE-activity. If the suitable database does not contain CRE-activity, then the CRE-activity of the nucleic acid sequences from the suitable database may be independently measured.

In example embodiments, the cell, tissue, or environment specific CRE-activity MPRA data set is obtained from a suitable database. In example embodiments, the CRE-activity database comprises UK Biobank and/or GTEx. The UK Biobank is a biomedical database and research resource, containing genetic and health information on half a million UK participants. The database is regularly updated and is globally accessible. The Genotype-Tissue Expression (GTEx) project is a public resource to study tissue-specific gene expression and regulation. GTEx provides open access to data including gene expression, QTLs, and histology images. Currently, samples have been collected from 54 non-diseased tissue sites across approximately 1000 individuals. These samples have been primarily used for molecular assays including WGS, WES, and RNA-Seq. The remaining samples are available in the GTEx Biobank.

In example embodiments, the CRE-activity data is derived from open epigenetic features such as DNase, H3K27ac, or ATAC seq.

### Unsupervised and Supervised Learning

In an example embodiment, unsupervised learning is implemented. Unsupervised learning can involve providing all or a portion of unlabeled training data to a machine learning module. The machine learning module can then determine one or more outputs implicitly based on the provided unlabeled training data. In an example embodiment, supervised learning is implemented. Supervised learning can involve providing all or a portion of labeled training data to a machine learning module, with the machine learning module determining one or more outputs based on the provided labeled training data, and the outputs are either accepted or corrected depending on the agreement to the actual outcome of the training data. In some examples, supervised learning of machine learning system(s) can be governed by a set of rules and/or a set of labels for the training input, and the set of rules and/or set of labels may be used to correct inferences of a machine learning module.

### Semi-Supervised and Reinforcement Learning

In one example embodiment, semi-supervised learning is implemented. Semi-supervised learning can involve providing all or a portion of training data that is partially labeled to a machine learning module. During semi-supervised learning, supervised learning is used for a portion of labeled training data, and unsupervised learning is used for a portion of unlabeled training data. In one example embodiment, reinforcement learning is implemented. Reinforcement learning can involve first providing all or a portion of the training data to a machine learning module and as the machine learning module produces an output, the machine learning module receives a “reward” signal in response to a correct output. Typically, the reward signal is a numerical value and the machine learning module is developed to maximize the numerical value of the reward signal. In addition, reinforcement learning can adopt a value function that provides a numerical value representing an expected total of the numerical values provided by the reward signal over time.

### Transfer Learning

In one example embodiment, transfer learning is implemented. Transfer learning techniques can involve providing all or a portion of a first training data to a machine learning module, then, after training on the first training data, providing all or a portion of a second training data. In example embodiments, a first machine learning module can be pre-trained on data from one or more computing devices. The first trained machine learning module is then provided to a computing device, where the computing device is intended to execute the first trained machine learning model to produce an output. Then, during the second training phase, the first trained machine learning model can be additionally trained using additional training data, where the training data can be derived from kernel and non-kernel data of one or more computing devices. This second training of the machine learning module and/or the first trained machine learning model using the training data can be performed using either supervised, unsupervised, or semi-supervised learning. In addition, it is understood transfer learning techniques can involve one, two, three, or more training attempts. Once the machine learning module has been trained on at least the training data, the training phase can be completed. The resulting trained machine learning model can be utilized as at least one of trained machine learning module.

### Incremental and Curriculum Learning

In one example embodiment, incremental learning is implemented. Incremental learning techniques can involve providing a trained machine learning module with input data that is used to continuously extend the knowledge of the trained machine learning module. Another machine learning training technique is curriculum learning, which can involve training the machine learning module with training data arranged in a particular order, such as providing relatively easy training examples first, then proceeding with progressively more difficult training examples. As the name suggests, difficulty of training data is analogous to a curriculum or course of study at a school.

### Learning to Learn

In one example embodiment, learning to learn is implemented. Learning to learn, or meta-learning, comprises, in general, two levels of learning: quick learning of a single task and slower learning across many tasks. For example, a machine learning module is first trained and comprises of a first set of parameters or weights. During or after operation of the first trained machine learning module, the parameters or weights are adjusted by the machine learning module. This process occurs iteratively on the success of the machine learning module. In another example, an optimizer, or another machine learning module, is used wherein the output of a first trained machine learning module is fed to an optimizer that constantly learns and returns the final results. Other techniques for training the machine learning module and/or trained machine learning module are possible as well.

### Contrastive Learning

In example embodiment, contrastive learning is implemented. Contrastive learning is a self-supervised model of learning in which training data is unlabeled is considered as a form of learning in-between supervised and unsupervised learning. This method learns by contrastive loss, which separates unrelated (i.e., negative) data pairs and connects related (i.e., positive) data pairs. For example, to create positive and negative data pairs, more than one view of a datapoint, such as rotating an image or using a different time-point of a video, is used as input. Positive and negative pairs are learned by solving dictionary look-up problem. The two views are separated into query and key of a dictionary. A query has a positive match to a key and negative match to all other keys. The machine learning module then learns by connecting queries to their keys and separating queries from their non-keys. A loss function, such as those described herein, is used to minimize the distance between positive data pairs (e.g., a query to its key) while maximizing the distance between negative data points. See e.g., Tian, Yonglong, et al. “What makes for good views for contrastive learning?.” Advances in Neural Information Processing Systems 33 (2020): 6827-6839.

### Pre-Trained Learning

In example embodiments, the machine learning module is pre-trained. A pre-trained machine learning model is a model that has been previously trained to solve a similar problem. The pre-trained machine learning model is generally pre-trained with similar input data to that of the new problem. A pre-trained machine learning model further trained to solve a new problem is generally referred to as transfer learning, which is described herein. In some instances, a pre-trained machine learning model is trained on a large dataset of related information. The pre-trained model is then further trained and tuned for the new problem. Using a pre-trained machine learning module provides the advantage of building a new machine learning module with input neurons/nodes that are already familiar with the input data and are more readily refined to a particular problem. For example, a machine learning module previously trained using accessible genomic sites mapped in 164 cell types by DNase-seq (e.g., Kelley, D. R., Snoek, J., & Rinn, J. L. (2016). Basset: Learning the regulatory code of the accessible genome with deep convolutional neural networks. Genome Research, 26 (7), 990-999) may be further trained to estimate CRE activity. See e.g., Diamant N, et al. Patient contrastive learning: A performant, expressive, and practical approach to electrocardiogram modeling. PLOS Comput Biol. 2022 Feb. 14; 18 (2):e1009862.

In some examples, after the training phase has been completed but before producing predictions expressed as outputs, a trained machine learning module can be provided to a computing device where a trained machine learning module is not already resident, in other words, after training phase has been completed, the trained machine learning module can be downloaded to a computing device. For example, a first computing device storing a trained machine learning module can provide the trained machine learning module to a second computing device. Providing a trained machine learning module to the second computing device may comprise one or more of communicating a copy of trained machine learning module to the second computing device, making a copy of trained machine learning module for the second computing device, providing access to trained machine learning module to the second computing device, and/or otherwise providing the trained machine learning system to the second computing device. In example embodiments, a trained machine learning module can be used by the second computing device immediately after being provided by the first computing device. In some examples, after a trained machine learning module is provided to the second computing device, the trained machine learning module can be installed and/or otherwise prepared for use before the trained machine learning module can be used by the second computing device.

After a machine learning model has been trained it can be used to output, estimate, infer, predict, generate, produce, or determine, for simplicity these terms will collectively be referred to as results. A trained machine learning module can receive input data and operably generate results. As such, the input data can be used as an input to the trained machine learning module for providing corresponding results to kernel components and non-kernel components. For example, a trained machine learning module can generate results in response to requests. In example embodiments, a trained machine learning module can be executed by a portion of other software. For example, a trained machine learning module can be executed by a result daemon to be readily available to provide results upon request.

In example embodiments, a machine learning module and/or trained machine learning module can be executed and/or accelerated using one or more computer processors and/or on-device co-processors. Such on-device co-processors can speed up training of a machine learning module and/or generation of results. In some examples, trained machine learning module can be trained, reside, and execute to provide results on a particular computing device, and/or otherwise can make results for the particular computing device.

Input data can include data from a computing device executing a trained machine learning module and/or input data from one or more computing devices. In example embodiments, a trained machine learning module can use results as input feedback. A trained machine learning module can also rely on past results as inputs for generating new results. In example embodiments, input data can comprise one or more nucleic acid sequences and, when provided to a trained machine learning module, results in output data such as CRE activity. As described above, the one or more nucleic acid sequences that provide CRE-activity may be passed to an objective function for further refinement. In the case of an iterative process the one or more nucleic acid sequences that either have CRE-activity or have CRE-activity and pass the objective function are modified and used as new input data for the machine learning.

### Algorithms

Different machine-learning algorithms have been contemplated to carry out the embodiments discussed herein. For example, linear regression (LiR), logistic regression (LoR), Bayesian networks (for example, naive-bayes), random forest (RF) (including decision trees), neural networks (NN) (also known as artificial neural networks), matrix factorization, a hidden Markov model (HMM), support vector machines (SVM), K-means clustering (KMC), K-nearest neighbor (KNN), a suitable statistical machine learning algorithm, and/or a heuristic machine learning system for classifying or evaluating one or more nucleic acid sequences.

### Linear Regression (LiR)

In one example embodiment, linear regression machine learning is implemented. LiR is typically used in machine learning to predict a result through the mathematical relationship between an independent and dependent variable, such as one or more nucleic acid sequences and CRE activity, respectively. A simple linear regression model would have one independent variable (x) and one dependent variable (y). A representation of an example mathematical relationship of a simple linear regression model would be y=mx+b. In this example, the machine learning algorithm tries variations of the tuning variables m and b to optimize a line that includes all the given training data.

The tuning variables can be optimized, for example, with a cost function. A cost function takes advantage of the minimization problem to identify the optimal tuning variables. The minimization problem preposes the optimal tuning variable will minimize the error between the predicted outcome and the actual outcome. An example cost function may comprise summing all the square differences between the predicted and actual output values and dividing them by the total number of input values and results in the average square error.

To select new tuning variables to reduce the cost function, the machine learning module may use, for example, gradient descent methods. An example gradient descent method comprises evaluating the partial derivative of the cost function with respect to the tuning variables. The sign and magnitude of the partial derivatives indicate whether the choice of a new tuning variable value will reduce the cost function, thereby optimizing the linear regression algorithm. A new tuning variable value is selected depending on a set threshold. Depending on the machine learning module, a steep or gradual negative slope is selected. Both the cost function and gradient descent can be used with other algorithms and modules mentioned throughout. For the sake of brevity, both the cost function and gradient descent are well known in the art and are applicable to other machine learning algorithms and may not be mentioned with the same detail.

LiR models may have many levels of complexity comprising one or more independent variables. Furthermore, in an LiR function with more than one independent variable, each independent variable may have the same one or more tuning variables or each, separately, may have their own one or more tuning variables. The number of independent variables and tuning variables will be understood to one skilled in the art for the problem being solved. In example embodiments, one or more nucleic acid sequences is used as the independent variables to train a LiR machine learning module, which, after training, is used to estimate, for example, CRE activity.

### Logistic Regression (LoR)

In one example embodiment, logestic regression machine learning is implemented. Logistic Regression, often considered a LiR type model, is typically used in machine learning to classify information, such as one or more nucleic acid sequences into categories such as CRE activity. LoR takes advantage of probability to predict an outcome from input data. However, what makes LoR different from a LiR is that LoR uses a more complex logistic function, for example a sigmoid function. In addition, the cost function can be a sigmoid function limited to a result between 0 and 1. For example, the sigmoid function can be of the form f(x)=1/(1+e^{-x}), where x represents some linear representation of input features and tuning variables. Similar to LiR, the tuning variable(s) of the cost function are optimized (typically by taking the log of some variation of the cost function) such that the result of the cost function, given variable representations of the input features, is a number between 0 and 1, preferably falling on either side of 0.5. As described in LiR, gradient descent may also be used in LoR cost function optimization and is an example of the process. In example embodiments, one or more nucleic acid sequences are used as the independent variables to train a LoR machine learning module, which, after training, is used to estimate, for example, CRE activity.

### Bayesian Network

In one example embodiment, a Bayesian Network is implemented. BNs are used in machine learning to make predictions through Bayesian inference from probabilistic graphical models. In BNs, input features are mapped onto a directed acyclic graph forming the nodes of the graph. The edges connecting the nodes contain the conditional dependencies between nodes to form a predicative model. For each connected node the probability of the input features resulting in the connected node is learned and forms the predictive mechanism. The nodes may comprise the same, similar or different probability functions to determine movement from one node to another. The nodes of a Bayesian network are conditionally independent of its non-descendants given its parents thus satisfying a local Markov property. This property affords reduced computations in larger networks by simplifying the joint distribution.

There are multiple methods to evaluate the inference, or predictability, in a BN but only two are mentioned for demonstrative purposes. The first method involves computing the joint probability of a particular assignment of values for each variable. The joint probability can be considered the product of each conditional probability and, in some instances, comprises the logarithm of that product. The second method is Markov chain Monte Carlo (MCMC), which can be implemented when the sample size is large. MCMC is a well-known class of sample distribution algorithms and will not be discussed in detail herein.

The assumption of conditional independence of variables forms the basis for Naïve Bayes classifiers. This assumption implies there is no correlation between different input features. As a result, the number of computed probabilities is significantly reduced as well as the computation of the probability normalization. While independence between features is rarely true, this assumption exchanges reduced computations for less accurate predictions, however the predictions are reasonably accurate. In example embodiments, one or more nucleic acid sequences are mapped to the BN graph to train the BN machine learning module, which, after training, is used to estimate CRE activity.

### Random Forest

In one example embodiment, random forest (RF) is implemented. RF consists of an ensemble of decision trees producing individual class predictions. The prevailing prediction from the ensemble of decision trees becomes the RF prediction. Decision trees are branching flowchart-like graphs comprising of the root, nodes, edges/branches, and leaves. The root is the first decision node from which feature information is assessed and from it extends the first set of edges/branches. The edges/branches contain the information of the outcome of a node and pass the information to the next node. The leaf nodes are the terminal nodes that output the prediction. Decision trees can be used for both classification as well as regression and is typically trained using supervised learning methods. Training of a decision tree is sensitive to the training data set. An individual decision tree may become over or under-fit to the training data and result in a poor predictive model. Random forest compensates by using multiple decision trees trained on different data sets. In example embodiments, one or more nucleic acid sequences are used to train the nodes of the decision trees of a RF machine learning module, which, after training, is used to estimate CRE activity.

### Gradient Boosting

In an example embodiment, gradient boosting is implemented. Gradient boosting is a method of strengthening the evaluation capability of a decision tree node. In general, a tree is fit on a modified version of an original data set. For example, a decision tree is first trained with equal weights across its nodes. The decision tree is allowed to evaluate data to identify nodes that are less accurate. Another tree is added to the model and the weights of the corresponding underperforming nodes are then modified in the new tree to improve their accuracy. This process is performed iteratively until the accuracy of the model has reached a defined threshold or a defined limit of trees has been reached. Less accurate nodes are identified by the gradient of a loss function. Loss functions must be differentiable such as a linear or logarithmic functions. The modified node weights in the new tree are selected to minimize the gradient of the loss function. In an example embodiment, a decision tree is implemented to determine a CRE activity and gradient boosting is applied to the tree to improve its ability to accurately determine the CRE activity.

### Neural Networks

In one example embodiment, Neural Networks are implemented. NNs are a family of statistical learning models influenced by biological neural networks of the brain. NNs can be trained on a relatively-large dataset (e.g., 50,000 or more) and used to estimate, approximate, or predict an output that depends on a large number of inputs/features. NNs can be envisioned as so-called “neuromorphic” systems of interconnected processor elements, or “neurons”, and exchange electronic signals, or “messages”. Similar to the so-called “plasticity” of synaptic neurotransmitter connections that carry messages between biological neurons, the connections in NNs that carry electronic “messages” between “neurons” are provided with numeric weights that correspond to the strength or weakness of a given connection. The weights can be tuned based on experience, making NNs adaptive to inputs and capable of learning. For example, an NN for predicting CRE-activity is defined by a set of input neurons that can be given input data such as one or more nucleic acid sequences. The input neuron weighs and transforms the input data and passes the result to other neurons, often referred to as “hidden” neurons. This is repeated until an output neuron is activated. The activated output neuron produces a result. In example embodiments, one or more nucleic acid sequences are used to train the neurons in a NN machine learning module, which, after training, is used to estimate CRE activity.

### Convolutional Autoencoder

In example embodiments, convolutional autoencoder (CAE) is implemented. A CAE is a type of neural network and comprises, in general, two main components. First, the convolutional operator that filters an input signal to extract features of the signal. Second, an autoencoder that learns a set of signals from an input and reconstructs the signal into an output. By combining these two components, the CAE learns the optimal filters that minimize reconstruction error resulting an improved output. CAEs are trained to only learn filters capable of feature extraction that can be used to reconstruct the input. Generally, convolutional autoencoders implement unsupervised learning. In example embodiments, the convolutional autoencoder is a variational convolutional autoencoder. In example embodiments, features from one or more nucleic acid sequences are used as an input signal into a CAE which reconstructs that signal into an output such as a CRE activity.

### Deep Learning

In example embodiments, deep learning is implemented. Deep learning expands the neural network by including more layers of neurons. A deep learning module is characterized as having three “macro” layers: (1) an input layer which takes in the input features, and fetches embeddings for the input, (2) one or more intermediate (or hidden) layers which introduces nonlinear neural net transformations to the inputs, and (3) a response layer which transforms the final results of the intermediate layers to the prediction. In example embodiments, one or more nucleic acid sequences are used to train the neurons of a deep learning module, which, after training, is used to estimate CRE activity.

### Convolutional Neural Network (CNN)

In an example embodiment, a convolutional neural network is implemented. CNNs is a class of NNs further attempting to replicate the biological neural networks, but of the animal visual cortex. CNNs process data with a grid pattern to learn spatial hierarchies of features. Wherein NNs are highly connected, sometimes fully connected, CNNs are connected such that neurons corresponding to neighboring data (e.g., pixels) are connected. This significantly reduces the number of weights and calculations each neuron must perform.

In general, input data, such one or more nucleic acid sequences, comprises of a multidimensional vector. A CNN, typically, comprises of three layers: convolution, pooling, and fully connected. The convolution and pooling layers extract features and the fully connected layer combines the extracted features into an output, such as CRE activity.

In particular, the convolutional layer comprises of multiple mathematical operations such as of linear operations, a specialized type being a convolution. The convolutional layer calculates the scalar product between the weights and the region connected to the input volume of the neurons. These computations are performed on kernels, which are reduced dimensions of the input vector. The kernels span the entirety of the input. The rectified linear unit (i.e., ReLu) applies an elementwise activation function (e.g., sigmoid function) on the kernels.

CNNs can optimized with hyperparameters. In general, there three hyperparameters are used: depth, stride, and zero-padding. Depth controls the number of neurons within a layer. Reducing the depth may increase the speed of the CNN but may also reduce the accuracy of the CNN. Stride determines the overlap of the neurons. Zero-padding controls the border padding in the input.

The pooling layer down-samples along the spatial dimensionality of the given input (i.e., convolutional layer output), reducing the number of parameters within that activation. As an example, kernels are reduced to dimensionalities of 2×2 with a stride of 2, which scales the activation map down to 25%. The fully connected layer uses inter-layer-connected neurons (i.e., neurons are only connected to neurons in other layers) to score the activations for classification and/or regression. Extracted features may become hierarchically more complex as one layer feeds its output into the next layer. See O'Shea, K.; Nash, R. An Introduction to Convolutional Neural Networks. arXiv 2015 and Yamashita, R., et al Convolutional neural networks: an overview and application in radiology. Insights Imaging 9, 611-629 (2018).

### Recurrent Neural Network (RNN)

In an example embodiment, a recurrent neural network is implemented. RNNs are class of NNs further attempting to replicate the biological neural networks of the brain. RNNs comprise of delay differential equations on sequential data or time series data to replicate the processes and interactions of the human brain. RNNs have “memory” wherein the RNN can take information from prior inputs to influence the current output. RNNs can process variable length sequences of inputs by using their “memory” or internal state information. Where NNs may assume inputs are independent from the outputs, the outputs of RNNs may be dependent on prior elements with the input sequence. For example, input such as one or more nucleic acid sequences is received by a RNN, which determines CRE activity. See Sherstinsky, Alex. “Fundamentals of recurrent neural network (RNN) and long short-term memory (LSTM) network.” Physica D: Nonlinear Phenomena 404 (2020): 132306.

### Long Short-Term Memory (LSTM)

In an example embodiment, a Long Short-term Memory is implemented. LSTM are a class of RNNs designed to overcome vanishing and exploding gradients. In RNNs, long term dependencies become more difficult to capture because the parameters or weights either do not change with training or fluctuate rapidly. This occurs when the RNN gradient exponentially decreases to zero, resulting in no change to the weights or parameters, or exponentially increases to infinity, resulting in large changes in the weights or parameters. This exponential effect is dependent on the number of layers and multiplicative gradient. LSTM overcomes the vanishing/exploding gradients by implementing “cells” within the hidden layers of the NN. The “cells” comprise three gates: an input gate, an output gate, and a forget gate. The input gate reduces error by controlling relevant inputs to update the current cell state. The output gate reduces error by controlling relevant memory content in the present hidden state. The forget gate reduces error by controlling whether prior cell states are put in “memory” or forgotten. The gates use activation functions to determine whether the data can pass through the gates. While one skilled in the art would recognize the use of any relevant activation function, example activation functions are sigmoid, tanh, and RELU. See Zhu, Xiaodan, et al. “Long short-term memory over recursive structures.” International Conference on Machine Learning. PMLR, 2015.

### Matrix Factorization

In example embodiments, Matrix Factorization is implemented. Matrix factorization machine learning exploits inherent relationships between two entities drawn out when multiplied together. Generally, the input features are mapped to a matrix F which is multiplied with a matrix R containing the relationship between the features and a predicted outcome. The resulting dot product provides the prediction. The matrix R is constructed by assigning random values throughout the matrix. In this example, two training matrices are assembled. The first matrix X contains training input features and the second matrix Z contains the known output of the training input features. First the dot product of R and X are computed and the square mean error, as one example method, of the result is estimated. The values in R are modulated and the process is repeated in a gradient descent style approach until the error is appropriately minimized. The trained matrix R is then used in the machine learning model. In example embodiments, one or more nucleic acid sequences are used to train the relationship matrix R in a matrix factorization machine learning module. After training, the relationship matrix R and input matrix F, which comprises vector representations of one or more nucleic acid sequences, results in the prediction matrix P comprising CRE activity.

### Hidden Markov Model

In example embodiments, a hidden Markov model is implemented. An HMM takes advantage of the statistical Markov model to predict an outcome. A Markov model assumes a Markov process, wherein the probability of an outcome is solely dependent on the previous event. In the case of HMM, it is assumed an unknown or “hidden” state is dependent on some observable event. An HMM comprises a network of connected nodes. Traversing the network is dependent on three model parameters: start probability; state transition probabilities; and observation probability. The start probability is a variable that governs, from the input node, the most plausible consecutive state. From there each node i has a state transition probability to node j. Typically the state transition probabilities are stored in a matrix Mᵢⱼ wherein the sum of the rows, representing the probability of state i transitioning to state j, equals 1. The observation probability is a variable containing the probability of output o occurring. These too are typically stored in a matrix Nₒⱼ wherein the probability of output o is dependent on state j. To build the model parameters and train the HMM, the state and output probabilities are computed. This can be accomplished with, for example, an inductive algorithm. Next, the state sequences are ranked on probability, which can be accomplished, for example, with the Viterbi algorithm. Finally, the model parameters are modulated to maximize the probability of a certain sequence of observations. This is typically accomplished with an iterative process wherein the neighborhood of states is explored, the probabilities of the state sequences are measured, and model parameters updated to increase the probabilities of the state sequences. In example embodiments, one or more nucleic acid sequences are used to train the nodes/states of the HMM machine learning module, which, after training, is used to estimate CRE activity.

### Support Vector Machine

In example embodiments, support vector machines are implemented. SVMs separate data into classes defined by n-dimensional hyperplanes (n-hyperplane) and are used in both regression and classification problems. Hyperplanes are decision boundaries developed during the training process of a SVM. The dimensionality of a hyperplane depends on the number of input features. For example, a SVM with two input features will have a linear (1-dimensional) hyperplane while a SVM with three input features will have a planer (2-dimensional) hyperplane. A hyperplane is optimized to have the largest margin or spatial distance from the nearest data point for each data type. In the case of simple linear regression and classification a linear equation is used to develop the hyperplane. However, when the features are more complex a kernel is used to describe the hyperplane. A kernel is a function that transforms the input features into higher dimensional space. Kernel functions can be linear, polynomial, a radial distribution function (or gaussian radial distribution function), or sigmoidal. In example embodiments, one or more nucleic acid sequences are used to train the linear equation or kernel function of the SVM machine learning module, which, after training, is used to estimate CRE activity.

### K-Means Clustering

In one example embodiment, K-means clustering is implemented. KMC assumes data points have implicit shared characteristics and “clusters” data within a centroid or “mean” of the clustered data points. During training, KMC adds a number of k centroids and optimizes its position around clusters. This process is iterative, where each centroid, initially positioned at random, is re-positioned towards the average point of a cluster. This process concludes when the centroids have reached an optimal position within a cluster. Training of a KMC module is typically unsupervised. In example embodiments, one or more nucleic acid sequences are used to train the centroids of a KMC machine learning module, which, after training, is used to estimate CRE activity.

### K-Nearest Neighbor

In one example embodiment, K-nearest neighbor is implemented. On a general level, KNN shares similar characteristics to KMC. For example, KNN assumes data points near each other share similar characteristics and computes the distance between data points to identify those similar characteristics but instead of k centroids, KNN uses k number of neighbors. The k in KNN represents how many neighbors will assign a data point to a class, for classification, or object property value, for regression. Selection of an appropriate number of k is integral to the accuracy of KNN. For example, a large k may reduce random error associated with variance in the data but increase error by ignoring small but significant differences in the data. Therefore, a careful choice of k is selected to balance overfitting and underfitting. Concluding whether some data point belongs to some class or property value k, the distance between neighbors is computed. Common methods to compute this distance are Euclidean, Manhattan or Hamming to name a few. In an embodiment, neighbors are given weights depending on the neighbor distance to scale the similarity between neighbors to reduce the error of edge neighbors of one class “out-voting” near neighbors of another class. In one example embodiment, k is 1 and a Markov model approach is utilized. In example embodiments, one or more nucleic acid sequences are used to train a KNN machine learning module, which, after training, is used to estimate CRE activity.

To perform one or more of its functionalities, the machine learning module may communicate with one or more other systems. For example, an integration system may integrate the machine learning module with one or more email servers, web servers, one or more databases, or other servers, systems, or repositories. In addition, one or more functionalities may require communication between a user and the machine learning module.

Any one or more of the module(s) described herein may be implemented using hardware (e.g., one or more processors of a computer/machine) or a combination of hardware and software. For example, any module described herein may configure a hardware processor (e.g., among one or more hardware processors of a machine) to perform the operations described herein for that module. In some example embodiments, any one or more of the modules described herein may comprise one or more hardware processors and may be configured to perform the operations described herein. In certain example embodiments, one or more hardware processors are configured to include any one or more of the modules described herein.

Moreover, any two or more of these modules may be combined into a single module, and the functions described herein for a single module may be subdivided among multiple modules. Furthermore, according to various example embodiments, modules described herein as being implemented within a single machine, database, or device may be distributed across multiple machines, databases, or devices. The multiple machines, databases, or devices are communicatively coupled to enable communications between the multiple machines, databases, or devices. The modules themselves are communicatively coupled (e.g., via appropriate interfaces) to each other and to various data sources, to allow information to be passed between the applications so as to allow the applications to share and access common data.

### Multimodal Translation

In an example embodiment, the machine learning module comprises multimodal translation (MT), also known as multimodal machine translation or multimodal neural machine translation. MT comprises of a machine learning module capable of receiving multiple (e.g. two or more) modalities. Typically, the multiple modalities comprise of information connected to each other.

In example embodiments, the MT may comprise of a machine learning method further described herein. In an example embodiment, the MT comprises a neural network, deep neural network, convolutional neural network, convolutional autoencoder, recurrent neural network, or an LSTM. For example, one or more nucleic acid sequences comprising multiple modalities from a source described herein is embedded as further described herein. The embedded data is then received by the machine learning module. The machine learning module processes the embedded data (e.g. encoding and decoding) through the multiple layers of architecture then determines the CRE-activity corresponding the modalities comprising the input. The machine learning methods further described herein may be engineered for MT wherein the inputs described herein comprise of multiple modalities of one or more nucleic acid sequences. See e.g. Sulubacak, U., Caglayan, O., Grönroos, SA. et al. Multimodal machine translation through visuals and speech. Machine Translation 34, 97-147 (2020) and Huang, Xun, et al. “Multimodal unsupervised image-to-image translation.” Proceedings of the European conference on computer vision (ECCV). 2018.

### Example Computing Device

FIG. 17 depicts a block diagram of a computing machine 2000 and a module 2050 in accordance with certain examples. The computing machine 2000 may comprise, but are not limited to, remote devices, work stations, servers, computers, general purpose computers, Internet/web appliances, hand-held devices, wireless devices, portable devices, wearable computers, cellular or mobile phones, personal digital assistants (PDAs), smart phones, smart watches, tablets, ultrabooks, netbooks, laptops, desktops, multi-processor systems, microprocessor-based or programmable consumer electronics, game consoles, set-top boxes, network PCs, mini-computers, and any machine capable of executing the instructions. The module 2050 may comprise one or more hardware or software elements configured to facilitate the computing machine 2000 in performing the various methods and processing functions presented herein. The computing machine 2000 may include various internal or attached components such as a processor 2010, system bus 2020, system memory 2030, storage media 2040, input/output interface 2060, and a network interface 2070 for communicating with a network 2080.

The computing machine 2000 may be implemented as a conventional computer system, an embedded controller, a laptop, a server, a mobile device, a smartphone, a set-top box, a kiosk, a router or other network node, a vehicular information system, one or more processors associated with a television, a customized machine, any other hardware platform, or any combination or multiplicity thereof. The computing machine 2000 may be a distributed system configured to function using multiple computing machines interconnected via a data network or bus system.

The one or more processor 2010 may be configured to execute code or instructions to perform the operations and functionality described herein, manage request flow and address mappings, and to perform calculations and generate commands. Such code or instructions could include, but is not limited to, firmware, resident software, microcode, and the like. The processor 2010 may be configured to monitor and control the operation of the components in the computing machine 2000. The processor 2010 may be a general purpose processor, a processor core, a multiprocessor, a reconfigurable processor, a microcontroller, a digital signal processor (“DSP”), an application specific integrated circuit (“ASIC”), tensor processing units (TPUs), a graphics processing unit (“GPU”), a field programmable gate array (“FPGA”), a programmable logic device (“PLD”), a radio-frequency integrated circuit (RFIC), a controller, a state machine, gated logic, discrete hardware components, any other processing unit, or any combination or multiplicity thereof. In example embodiments, each processor 2010 can include a reduced instruction set computer (RISC) microprocessor. The processor 2010 may be a single processing unit, multiple processing units, a single processing core, multiple processing cores, special purpose processing cores, co-processors, or any combination thereof. According to certain examples, the processor 2010 along with other components of the computing machine 2000 may be a virtualized computing machine executing within one or more other computing machines. Processors 2010 are coupled to system memory and various other components via a system bus 2020.

The system memory 2030 may include non-volatile memories such as read-only memory (“ROM”), programmable read-only memory (“PROM”), erasable programmable read-only memory (“EPROM”), flash memory, or any other device capable of storing program instructions or data with or without applied power. The system memory 2030 may also include volatile memories such as random-access memory (“RAM”), static random-access memory (“SRAM”), dynamic random-access memory (“DRAM”), and synchronous dynamic random-access memory (“SDRAM”). Other types of RAM also may be used to implement the system memory 2030. The system memory 2030 may be implemented using a single memory module or multiple memory modules. While the system memory 2030 is depicted as being part of the computing machine 2000, one skilled in the art will recognize that the system memory 2030 may be separate from the computing machine 2000 without departing from the scope of the subject technology. It should also be appreciated that the system memory 2030 is coupled to system bus 2020 and can include a basic input/output system (BIOS), which controls certain basic functions of the processor 2010 and/or operate in conjunction with, a non-volatile storage device such as the storage media 2040.

In example embodiments, the computing device 2000 includes a graphics processing unit (GPU) 2090. Graphics processing unit 2090 is a specialized electronic circuit designed to manipulate and alter memory to accelerate the creation of images in a frame buffer intended for output to a display. In general, a graphics processing unit 2090 is efficient at manipulating computer graphics and image processing and has a highly parallel structure that makes it more effective than general-purpose CPUs for algorithms where processing of large blocks of data is done in parallel.

The storage media 2040 may include a hard disk, a floppy disk, a compact disc read only memory (“CD-ROM”), a digital versatile disc (“DVD”), a Blu-ray disc, a magnetic tape, a flash memory, other non-volatile memory device, a solid state drive (“SSD”), any magnetic storage device, any optical storage device, any electrical storage device, any electromagnetic storage device, any semiconductor storage device, any physical-based storage device, any removable and non-removable media, any other data storage device, or any combination or multiplicity thereof. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any other data storage device, or any combination or multiplicity thereof. The storage media 2040 may store one or more operating systems, application programs and program modules such as module 2050, data, or any other information. The storage media 2040 may be part of, or connected to, the computing machine 2000. The storage media 2040 may also be part of one or more other computing machines that are in communication with the computing machine 2000 such as servers, database servers, cloud storage, network attached storage, and so forth. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

The module 2050 may comprise one or more hardware or software elements, as well as an operating system, configured to facilitate the computing machine 2000 with performing the various methods and processing functions presented herein. The module 2050 may include one or more sequences of instructions stored as software or firmware in association with the system memory 2030, the storage media 2040, or both. The storage media 2040 may therefore represent examples of machine or computer readable media on which instructions or code may be stored for execution by the processor 2010. Machine or computer readable media may generally refer to any medium or media used to provide instructions to the processor 2010. Such machine or computer readable media associated with the module 2050 may comprise a computer software product. Each of the operating system, one or more application programs, other program modules, and program data or some combination thereof, may include an implementation of a networking environment. It should be appreciated that a computer software product comprising the module 2050 may also be associated with one or more processes or methods for delivering the module 2050 to the computing machine 2000 via the network 2080, any signal-bearing medium, or any other communication or delivery technology. The module 2050 may also comprise hardware circuits or information for configuring hardware circuits such as microcode or configuration information for an FPGA or other PLD.

The input/output (“I/O”) interface 2060 may be configured to couple to one or more external devices, to receive data from the one or more external devices, and to send data to the one or more external devices. Such external devices along with the various internal devices may also be known as peripheral devices. The I/O interface 2060 may include both electrical and physical connections for coupling in operation the various peripheral devices to the computing machine 2000 or the processor 2010. The I/O interface 2060 may be configured to communicate data, addresses, and control signals between the peripheral devices, the computing machine 2000, or the processor 2010. The I/O interface 2060 may be configured to implement any standard interface, such as small computer system interface (“SCSI”), serial-attached SCSI (“SAS”), fiber channel, peripheral component interconnect (“PCI”), PCI express (PCIe), serial bus, parallel bus, advanced technology attached (“ATA”), serial ATA (“SATA”), universal serial bus (“USB”), Thunderbolt, FireWire, various video buses, and the like. The I/O interface 2060 may be configured to implement only one interface or bus technology. Alternatively, the I/O interface 2060 may be configured to implement multiple interfaces or bus technologies. The I/O interface 2060 may be configured as part of, all of, or to operate in conjunction with, the system bus 2020. The I/O interface 2060 may include one or more buffers for buffering transmissions between one or more external devices, internal devices, the computing machine 2000, or the processor 2010.

The I/O interface 2060 may couple the computing machine 2000 to various input devices including cursor control devices, touch-screens, scanners, electronic digitizers, sensors, receivers, touchpads, trackballs, cameras, microphones, alphanumeric input devices, any other pointing devices, or any combinations thereof. The I/O interface 2060 may couple the computing machine 2000 to various output devices including video displays (The computing device 2000 may further include a graphics display, for example, a plasma display panel (PDP), a light emitting diode (LED) display, a liquid crystal display (LCD), a projector, a cathode ray tube (CRT), or any other display capable of displaying graphics or video), audio generation device, printers, projectors, tactile feedback devices, automation control, robotic components, actuators, motors, fans, solenoids, valves, pumps, transmitters, signal emitters, lights, and so forth. The I/O interface 2060 may couple the computing device 2000 to various devices capable of input and out, such as a storage unit. The devices can be interconnected to the system bus 2020 via a user interface adapter, which can include, for example, a Super I/O chip integrating multiple device adapters into a single integrated circuit.

The computing machine 2000 may operate in a networked environment using logical connections through the network interface 2070 to one or more other systems or computing machines across the network 2080. The network 2080 may include a local area network (“LAN”), a wide area network (“WAN”), an intranet, an Internet, a mobile telephone network, storage area network (“SAN”), personal area network (“PAN”), a metropolitan area network (“MAN”), a wireless network (“WiFi;”), wireless access networks, a wireless local area network (“WLAN”), a virtual private network (“VPN”), a cellular or other mobile communication network, Bluetooth, near field communication (“NFC”), ultra-wideband, wired networks, telephone networks, optical networks, copper transmission cables, or combinations thereof or any other appropriate architecture or system that facilitates the communication of signals and data. The network 2080 may be packet switched, circuit switched, of any topology, and may use any communication protocol. The network 2080 may comprise routers, firewalls, switches, gateway computers and/or edge servers. Communication links within the network 2080 may involve various digital or analog communication media such as fiber optic cables, free-space optics, waveguides, electrical conductors, wireless links, antennas, radio-frequency communications, and so forth.

Information for facilitating reliable communications can be provided, for example, as packet/message sequencing information, encapsulation headers and/or footers, size/time information, and transmission verification information such as cyclic redundancy check (CRC) and/or parity check values. Communications can be made encoded/encrypted, or otherwise made secure, and/or decrypted/decoded using one or more cryptographic protocols and/or algorithms, such as, but not limited to, Data Encryption Standard (DES), Advanced Encryption Standard (AES), a Rivest-Shamir-Adelman (RSA) algorithm, a Diffie-Hellman algorithm, a secure sockets protocol such as Secure Sockets Layer (SSL) or Transport Layer Security (TLS), and/or Digital Signature Algorithm (DSA). Other cryptographic protocols and/or algorithms can be used as well or in addition to those listed herein to secure and then decrypt/decode communications.

The processor 2010 may be connected to the other elements of the computing machine 2000 or the various peripherals discussed herein through the system bus 2020. The system bus 2020 represents one or more of any of several types of bus structures, including a memory bus or memory controller, a peripheral bus, an accelerated graphics port, and a processor or local bus using any of a variety of bus architectures. For example, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus. It should be appreciated that the system bus 2020 may be within the processor 2010, outside the processor 2010, or both. According to certain examples, any of the processor 2010, the other elements of the computing machine 2000, or the various peripherals discussed herein may be integrated into a single device such as a system on chip (“SOC”), system on package (“SOP”), or ASIC device.

Examples may comprise a computer program that embodies the functions described and illustrated herein, wherein the computer program is implemented in a computer system that comprises instructions stored in a machine-readable medium and a processor that executes the instructions. However, it should be apparent that there could be many different ways of implementing examples in computer programming, and the examples should not be construed as limited to any one set of computer program instructions. Further, a skilled programmer would be able to write such a computer program to implement an example of the disclosed examples based on the appended flow charts and associated description in the application text. Therefore, disclosure of a particular set of program code instructions is not considered necessary for an adequate understanding of how to make and use examples. Further, those ordinarily skilled in the art will appreciate that one or more aspects of examples described herein may be performed by hardware, software, or a combination thereof, as may be embodied in one or more computing systems. Moreover, any reference to an act being performed by a computer should not be construed as being performed by a single computer as more than one computer may perform the act.

The examples described herein can be used with computer hardware and software that perform the methods and processing functions described herein. The systems, methods, and procedures described herein can be embodied in a programmable computer, computer-executable software, or digital circuitry. The software can be stored on computer-readable media. For example, computer-readable media can include a floppy disk, RAM, ROM, hard disk, removable media, flash memory, memory stick, optical media, magneto-optical media, CD-ROM, etc. Digital circuitry can include integrated circuits, gate arrays, building block logic, field programmable gate arrays (FPGA), etc.

A “server” may comprise a physical data processing system (for example, the computing device 2000 as shown in FIG. 17) running a server program. A physical server may or may not include a display and keyboard. A physical server may be connected, for example by a network, to other computing devices. Servers connected via a network may operate in the capacity of a server machine or a client machine in a server-client network environment, or as a peer machine in a distributed (e.g., peer-to-peer) network environment. The computing device 2000 can include clients' servers. For example, a client and server can be remote from each other and interact through a network. The relationship of client and server arises by virtue of computer programs in communication with each other, running on the respective computers.

Any two or more devices, two or more software/programs, and any two or more portions of a device or software/program, for simplicity referred to as technology, may be described herein as operably linked. Operably linked may be defined as at least one technology can mediate a function exerted upon at least one other technology such that the two or more technologies function normally. In general, operably linked refers to the ability for at least one technology to communicate with at least one other technology.

The example systems, methods, and acts described in the examples and described in the figures presented previously are illustrative, not intended to be exhaustive, and not meant to be limiting. In alternative examples, certain acts can be performed in a different order, in parallel with one another, omitted entirely, and/or combined between different examples, and/or certain additional acts can be performed, without departing from the scope and spirit of various examples. Plural instances may implement components, operations, or structures described as a single instance. Structures and functionality that may appear as separate in example embodiments may be implemented as a combined structure or component. Similarly, structures and functionality that may appear as a single component may be implemented as separate components. Accordingly, such alternative examples are included in the scope of the following claims, which are to be accorded the broadest interpretation to encompass such alternate examples. The terminology used herein was chosen to best explain the principles of the embodiments, the practical application or technical improvement over technologies found in the marketplace, or to enable others of ordinary skill in the art to understand the embodiments disclosed herein.

### Cis-Regulatory Elements (CREs)

Described in certain example embodiments herein are CREs. In an embodiment, the CREs are identified or engineered using a computer implemented method for identifying CREs and/or designing engineered CREs with a specific activity (e.g., a cell type, cell state, tissue type, and/or environmental specificity or specific activity) of the present invention as described in greater detail elsewhere herein.

In an embodiment the CRE is identified or designed using a method, such as a computer implemented method of the present invention described in greater detail elsewhere herein. In an embodiment, the CRE is an engineered CRE. In an embodiment, the CRE is an identified CRE. In an embodiment, the CRE comprises two or more (e.g., 2, 3, 4, 5, 6, 7, 8, 9, 10 or more) CREs designed using computer implemented method of the present invention described in greater detail elsewhere herein. In an embodiment, one or more of the two or more CREs are an engineered CRE.

In an embodiment, the engineered CRE is cell type, cell state, tissue type, and/or environment specific. In an embodiment, the identified CRE is cell type, cell state, tissue type, and/or environment specific.

In an embodiment, the engineered CRE does not have a significant match in a genome of an organism. In an embodiment, the organism is a vertebrate or invertebrate. In an embodiment, the organism is a mammal, avian, reptile, fish, or amphibian. In an embodiment, the organism is a human or non-human primate. In an embodiment, the organism is a plant. In an embodiment, one or more CREs, optionally one or more engineered CREs, is/are specific for a diseased or abnormal cell type and/or cell state.

In an embodiment, one or more identified and/or engineered CREs are cell-type specific and/or tissue specific CREs. In other words, In an embodiment, one or more CREs have cell type specificity (i.e., specific activity) and/or tissue type specificity. In an embodiment, one or more identified and/or engineered CREs are cell state specific CREs. In other words, In an embodiment, one or more CREs have cell state specificity (i.e., specific activity). In an embodiment, one or more identified and/or engineered CREs are environmental specific CREs. In other words, In an embodiment, one or more CREs have an environmental specificity (i.e., specific activity). Environment here refers to an environment internal or external to a cell. In an embodiment, one or more CREs can be specific to one or more attributes to an internal or external cellular environment, such as an energy (e.g., light, acoustic, magnetic, electromagnetic, or other energy), chemical, or biological stimuli, an osmolarity, heat, cold, radiation, salinity, pressure, strain, humidity, gas content (e.g., partial pressure of CO₂, CO, NO, O₂, etc.), or other internal or external environmental condition.

In an embodiment, the engineered CRE is or contains a polynucleotide set forth in Supplementary Table 2 of Gosai et al. “Machine-guided design of synthetic cell type-specific cis-regulatory elements” BioRxiv doi: doi.org/10.1101/2023.08.08.552077 (2023). In an embodiment, the engineered CRE is or contains a polynucleotide set forth in Supplementary Table 10 of Gosai et al. “Machine-guided design of synthetic cell type-specific cis-regulatory elements” BioRxiv doi: doi.org/10.1101/2023.08.08.552077 (2023).

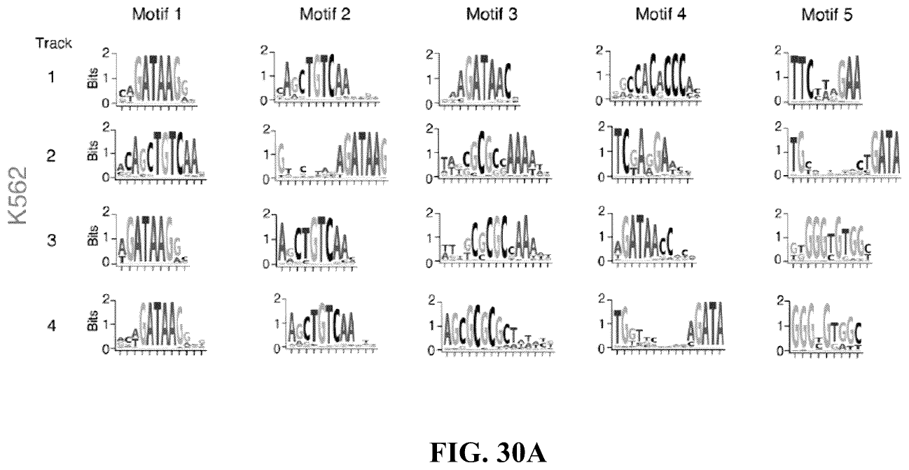

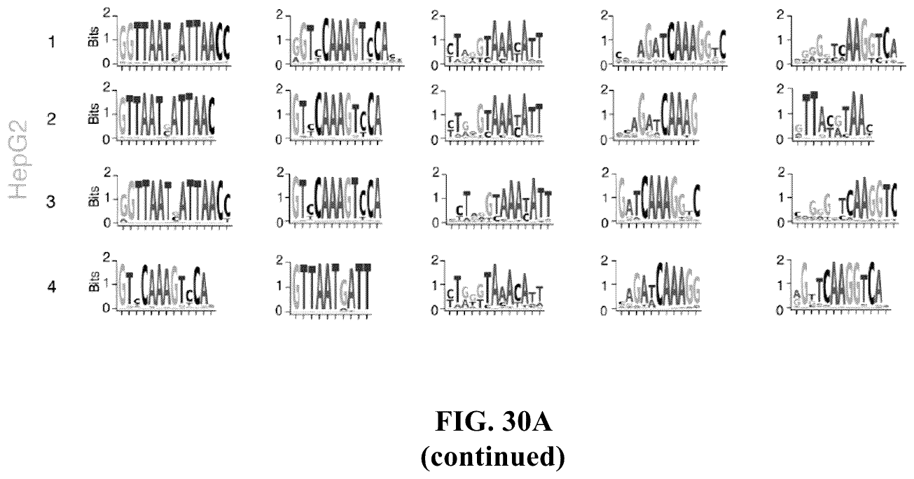

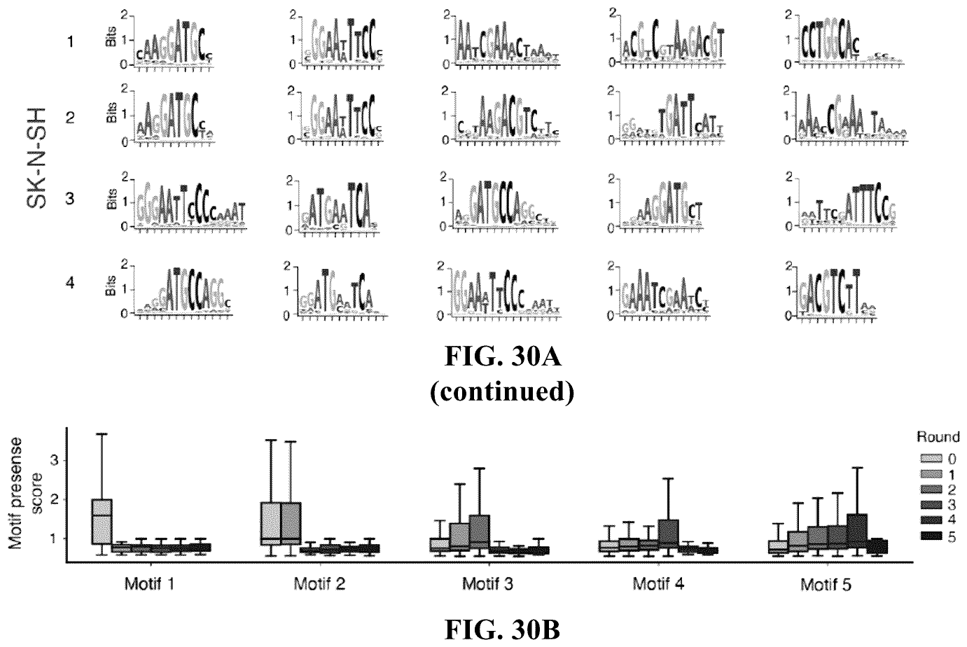

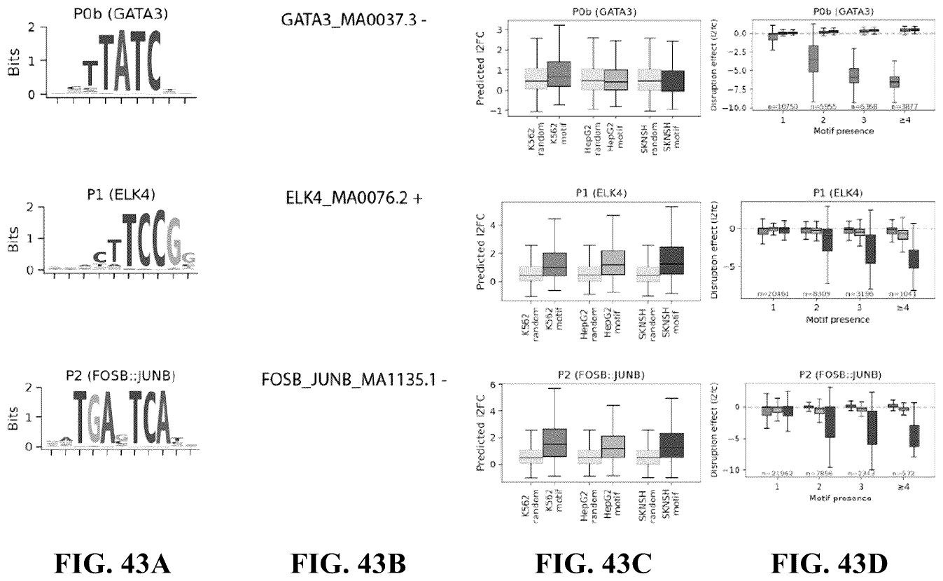

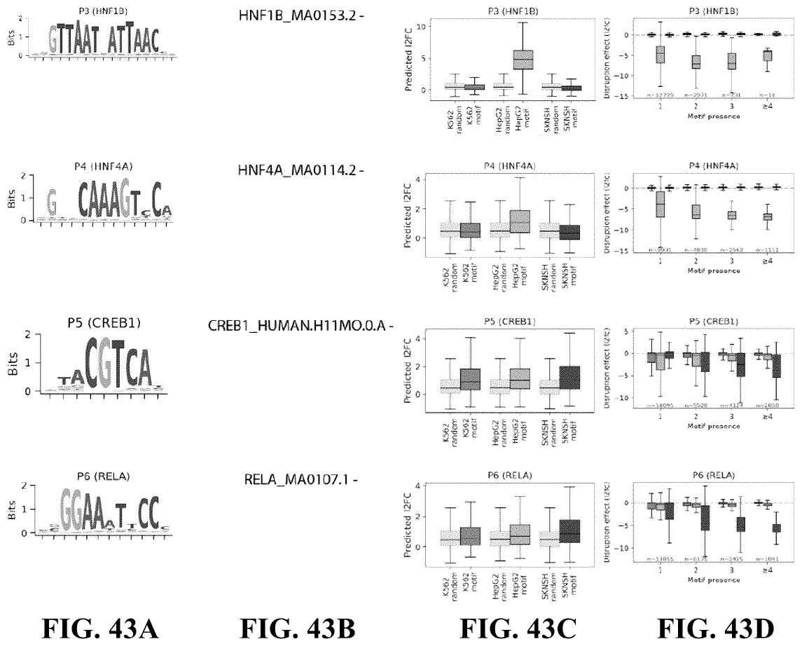

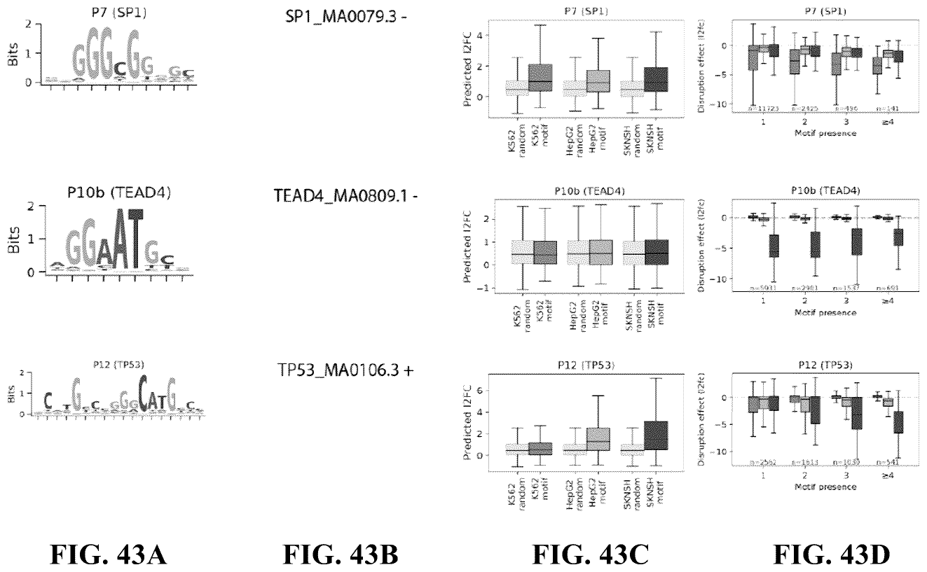

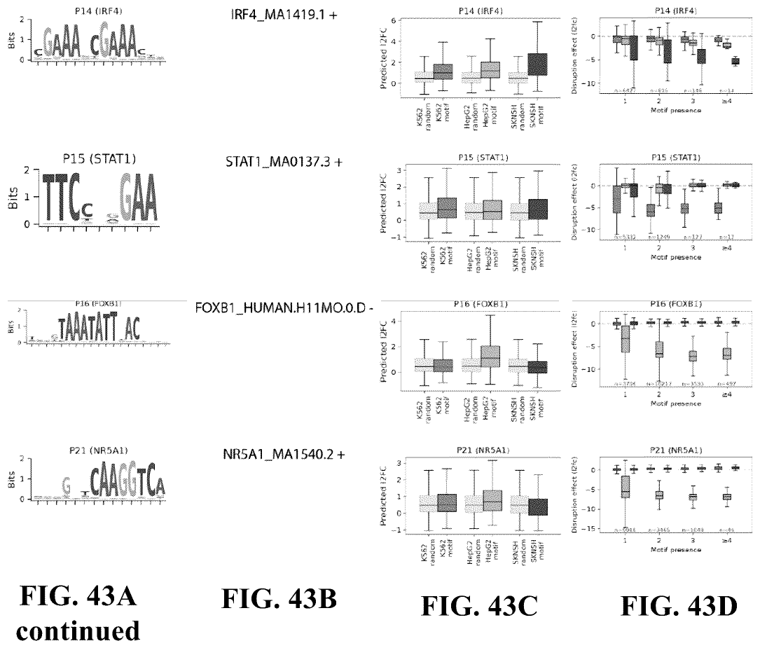

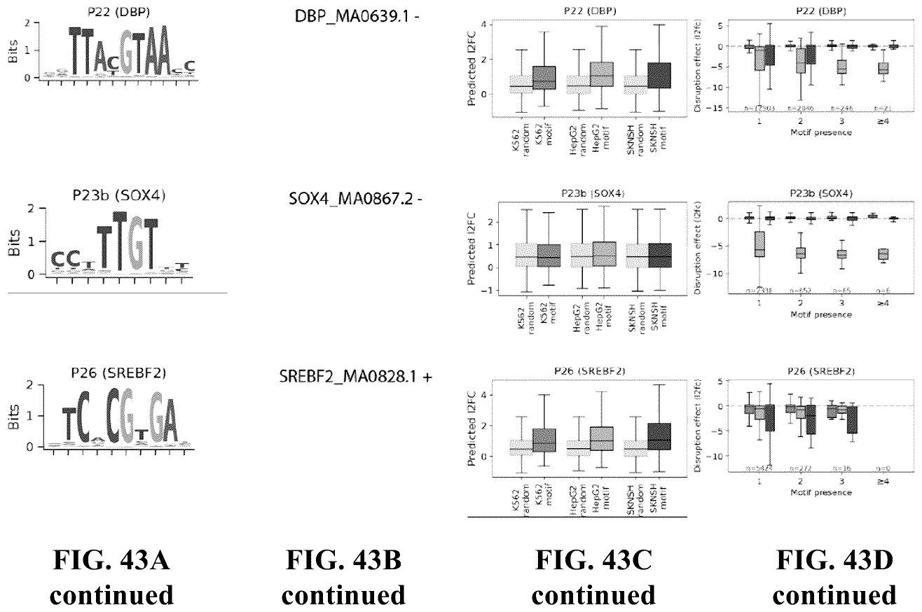

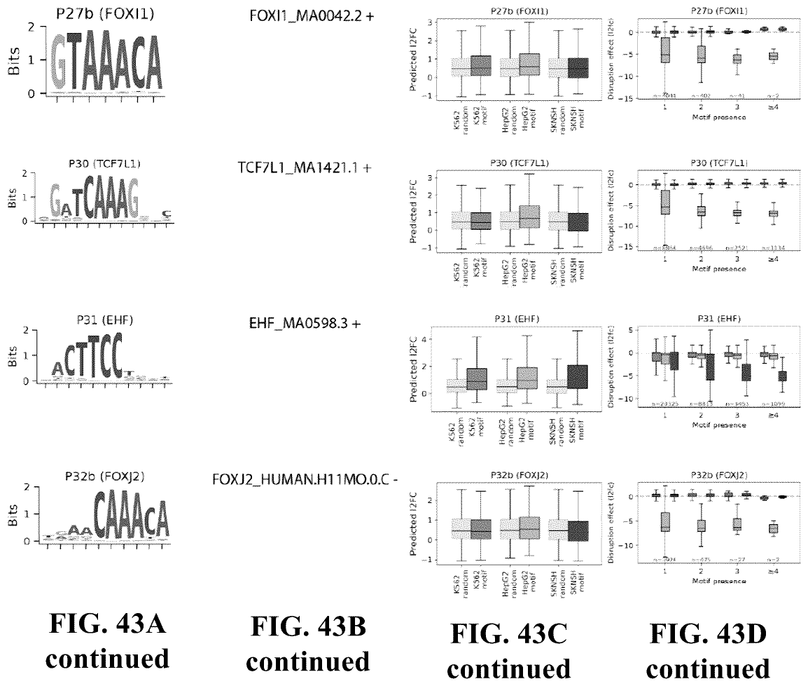

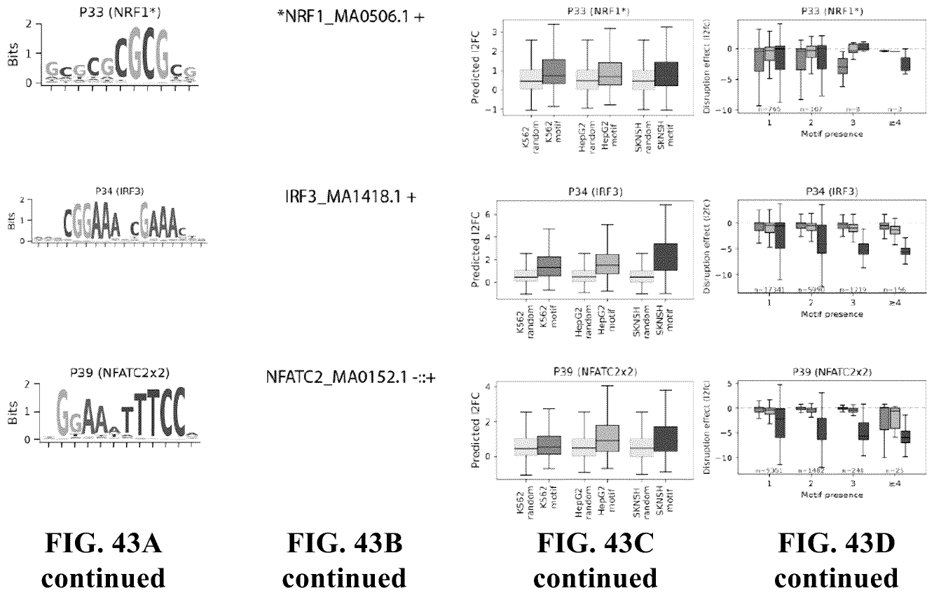

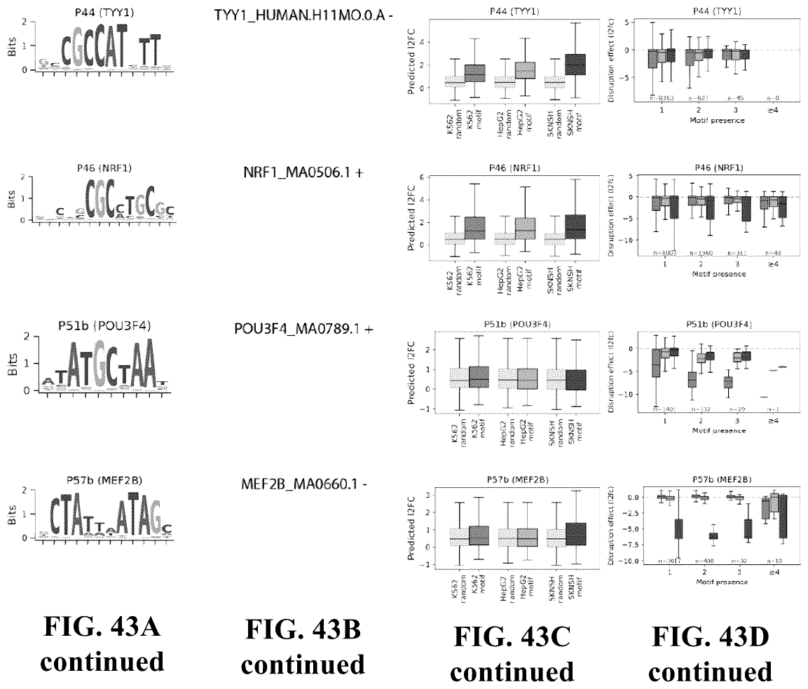

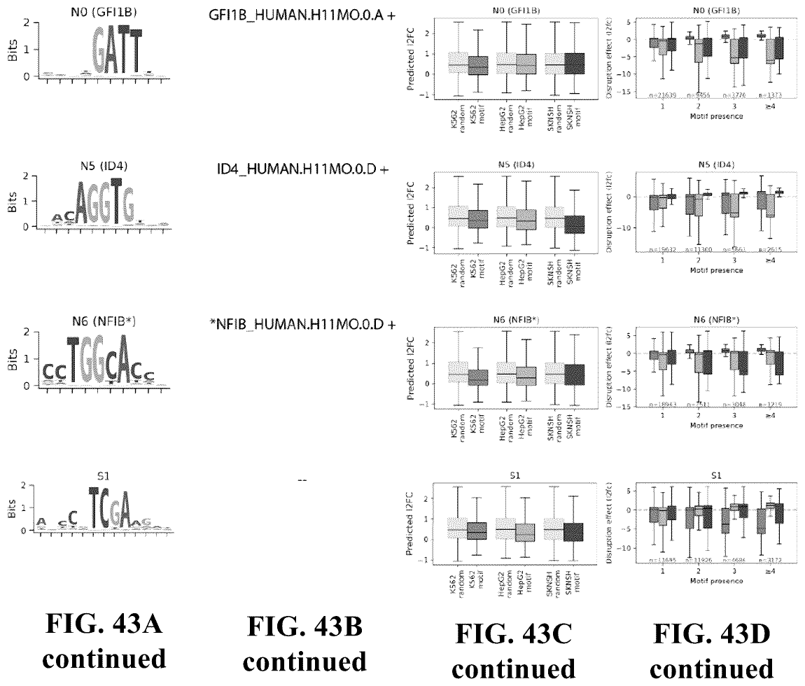

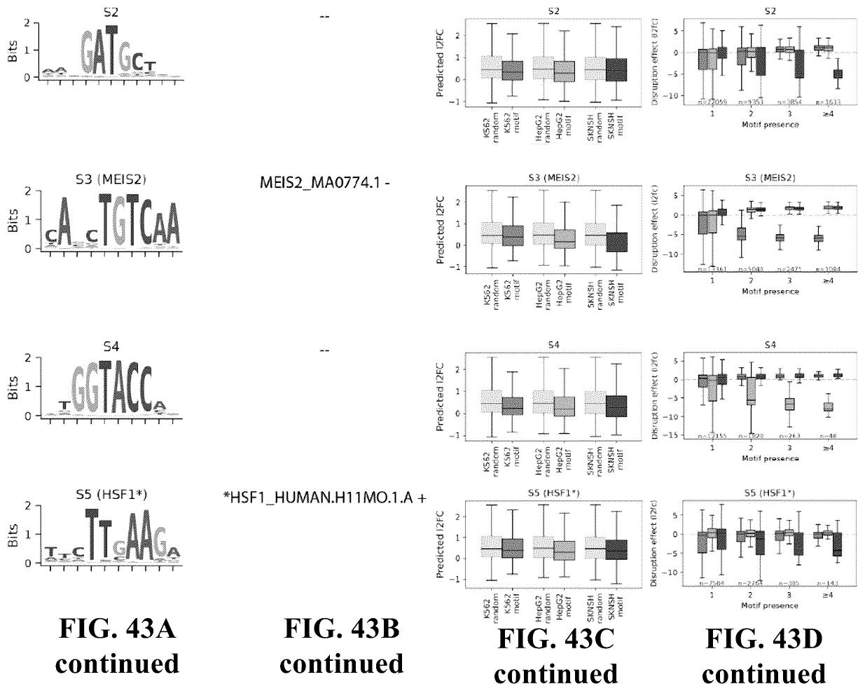

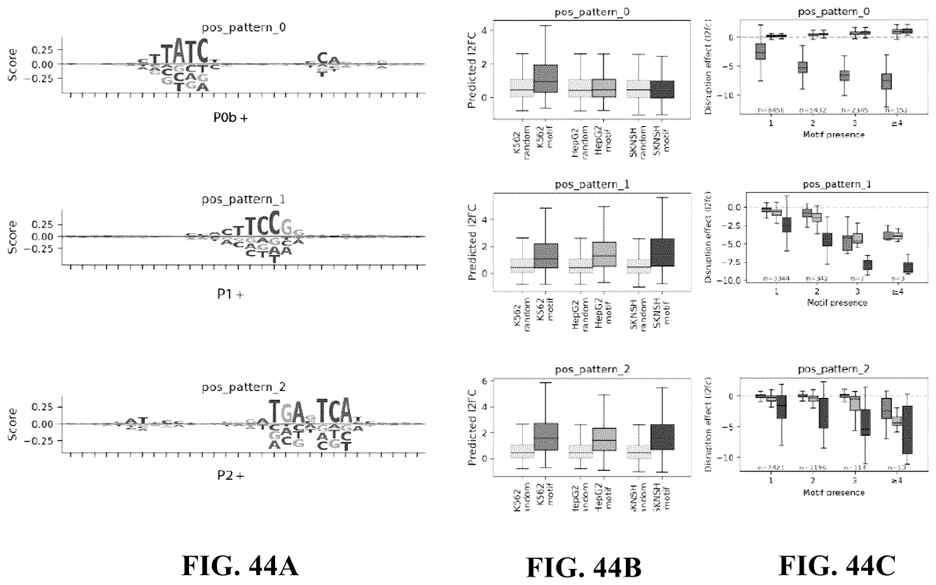

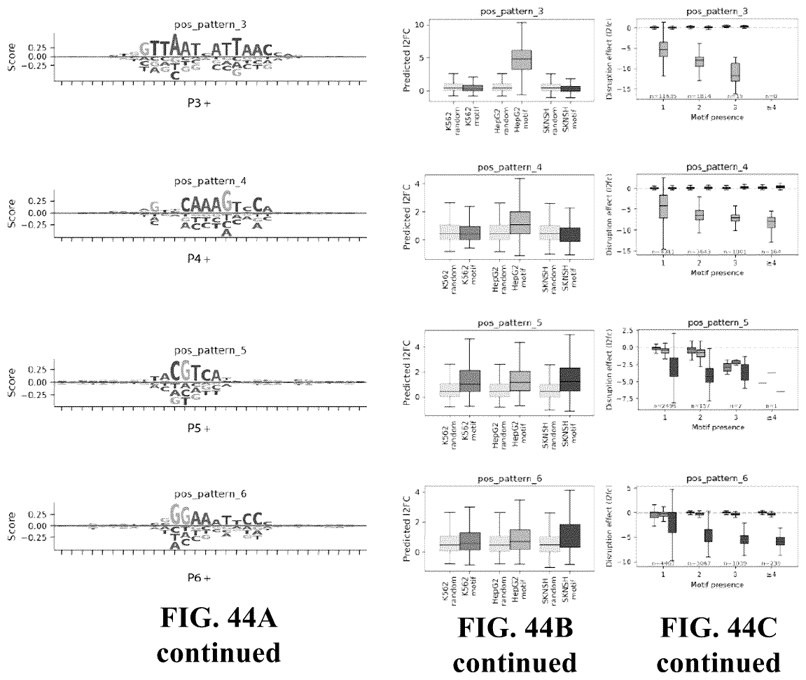

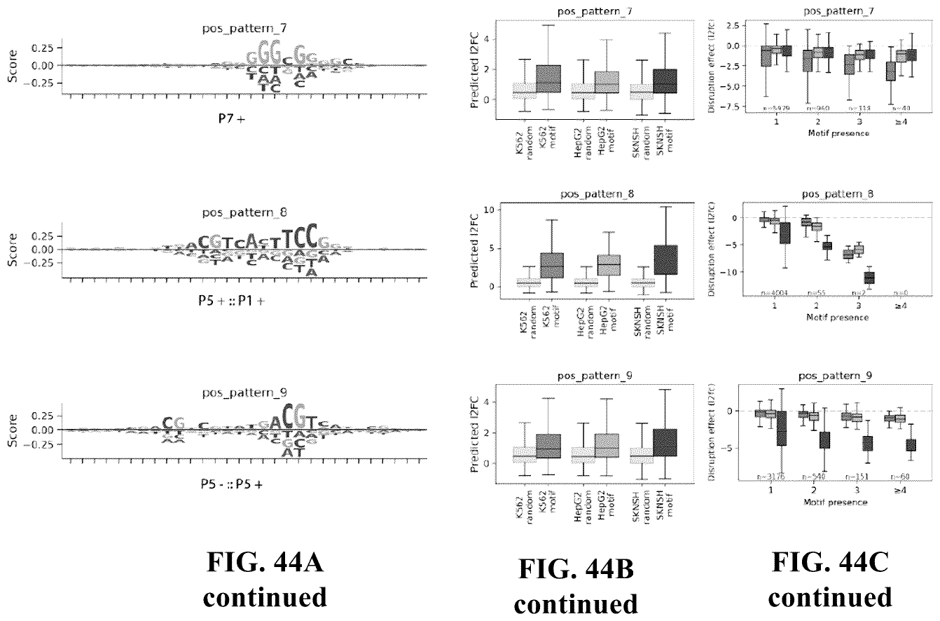

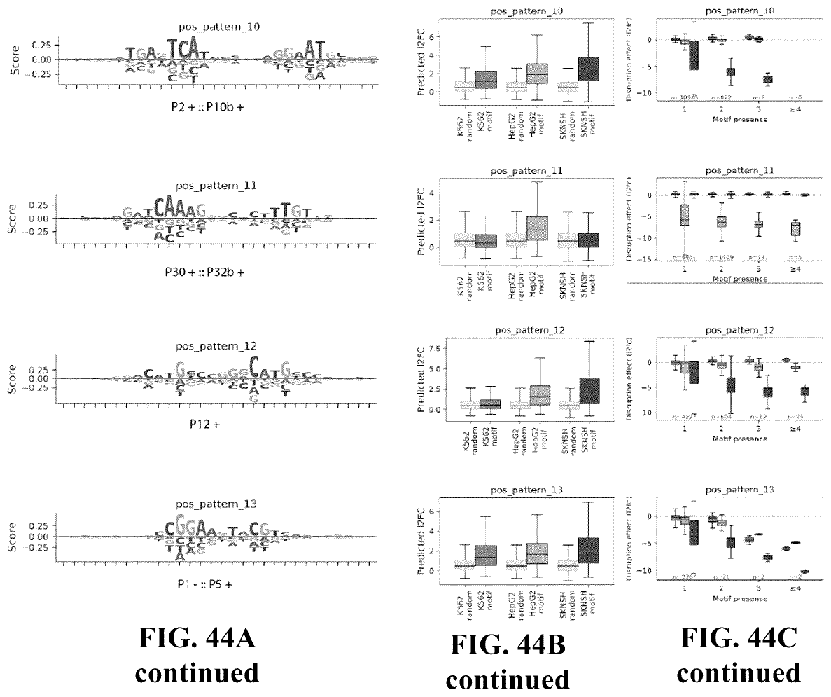

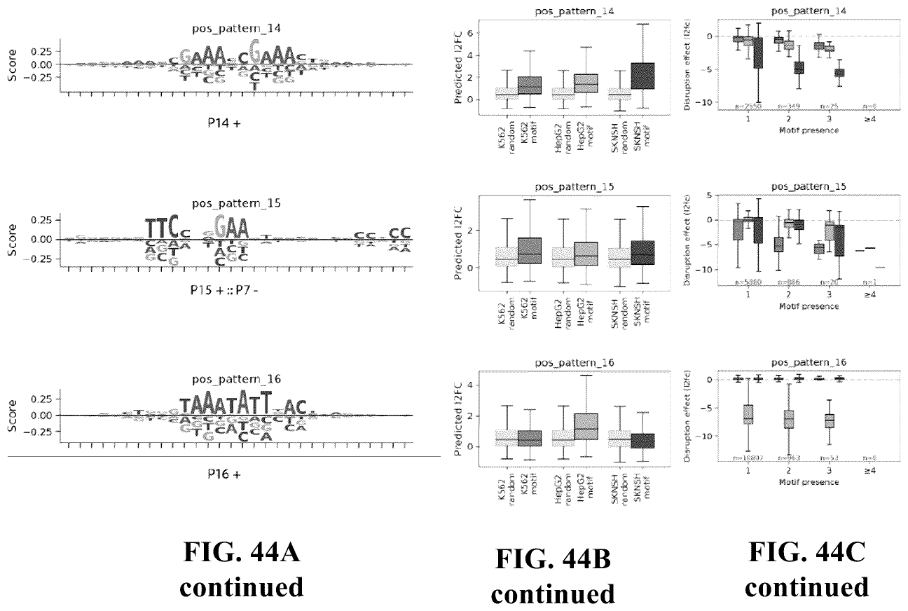

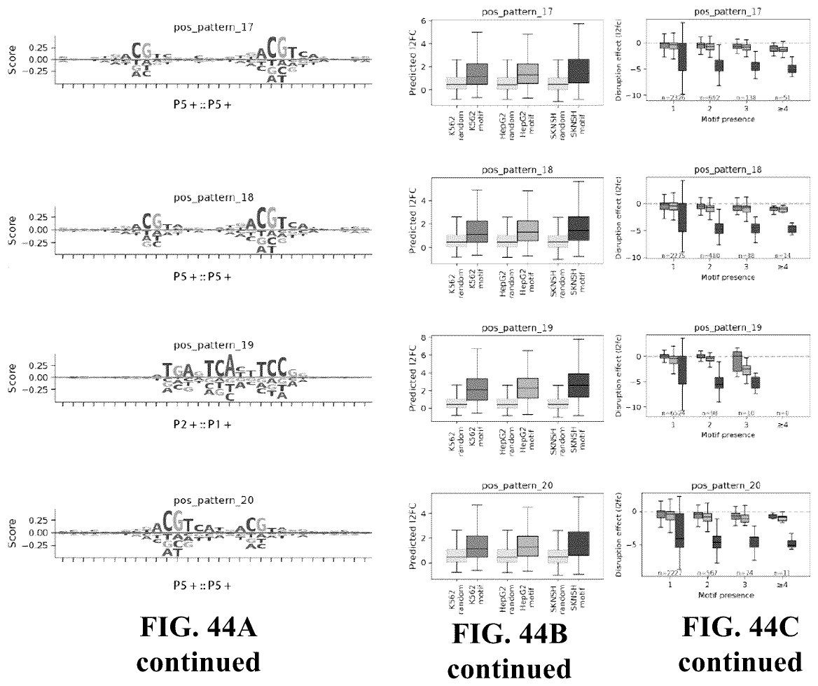

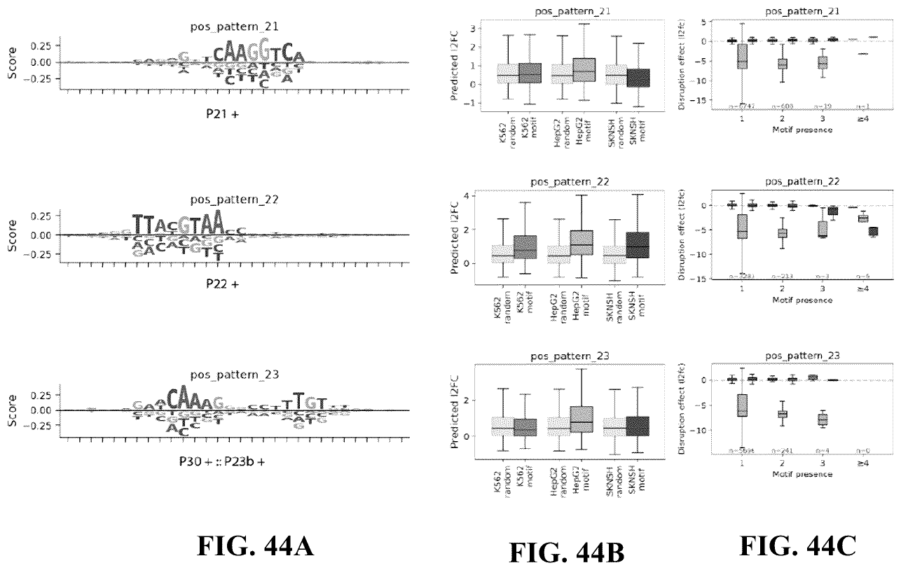

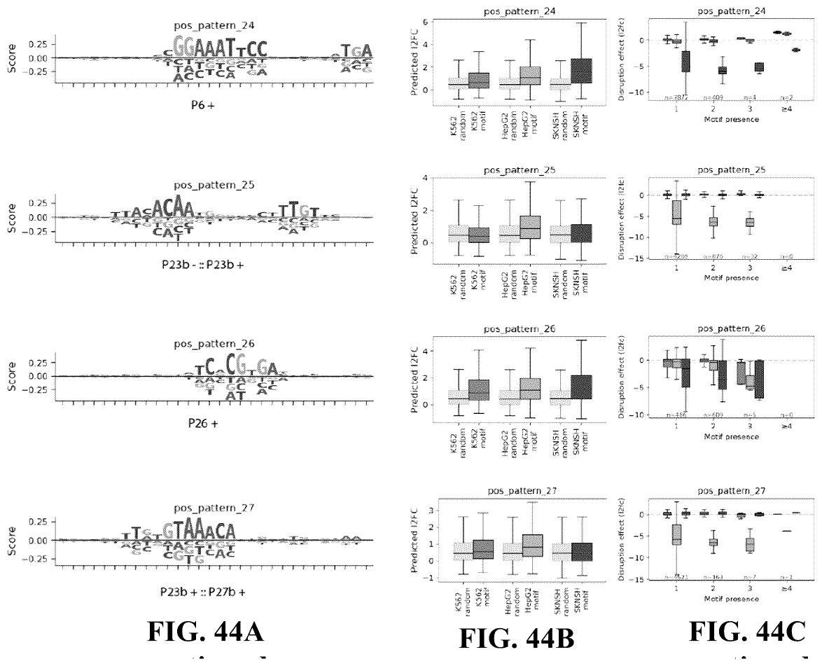

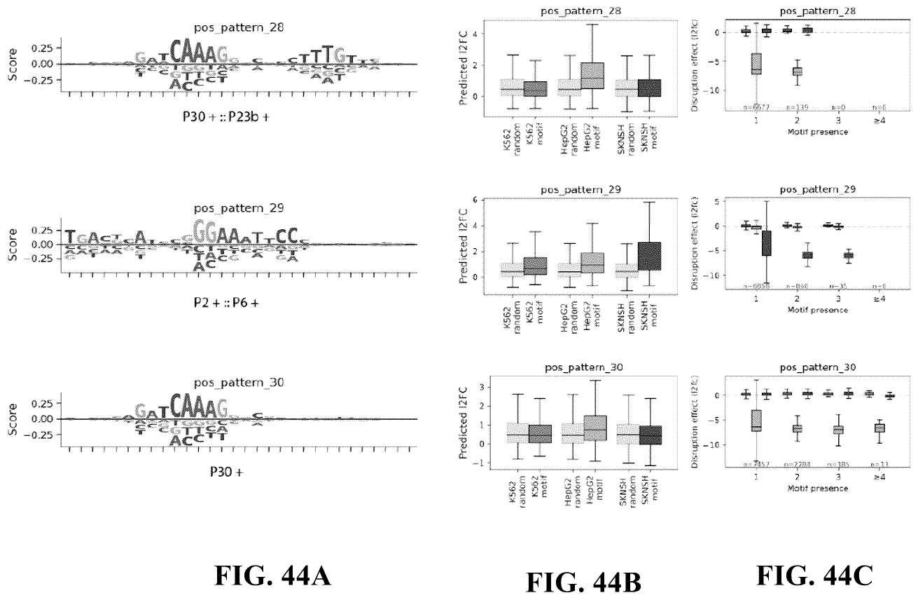

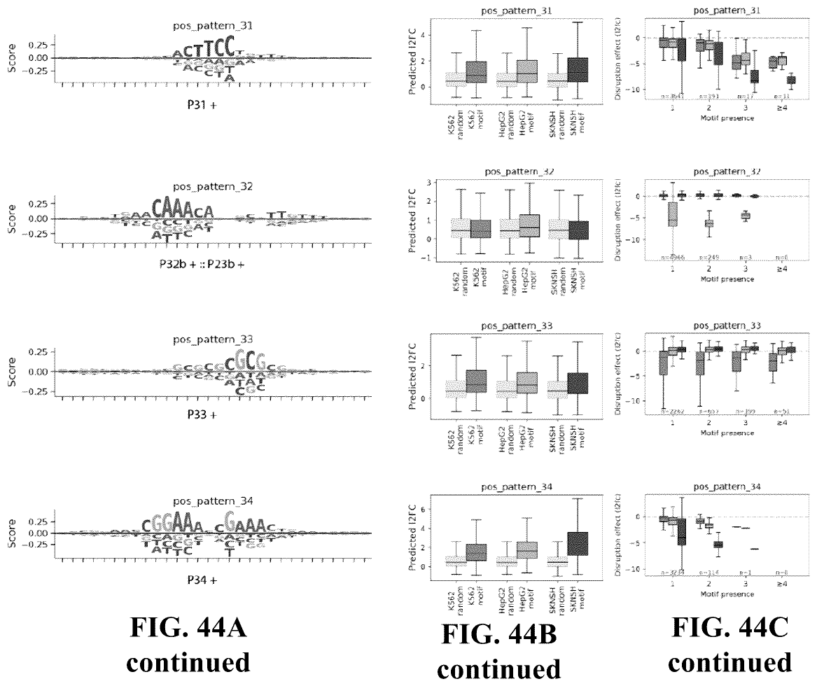

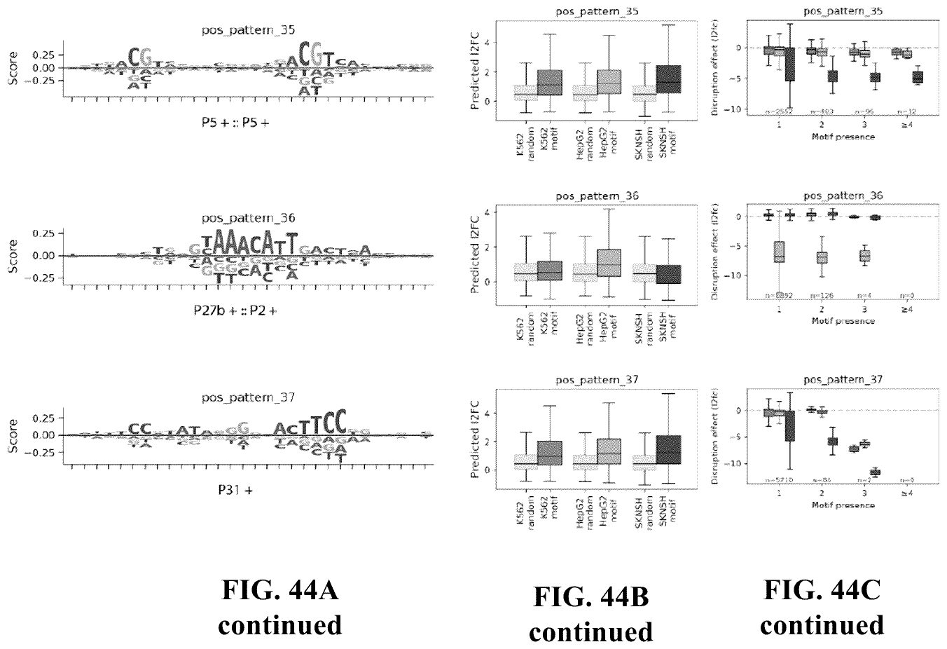

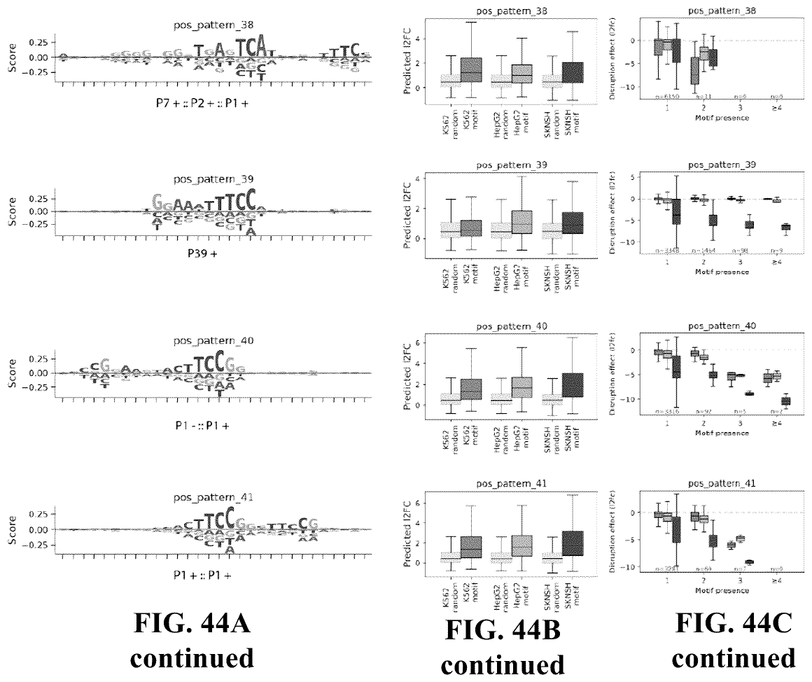

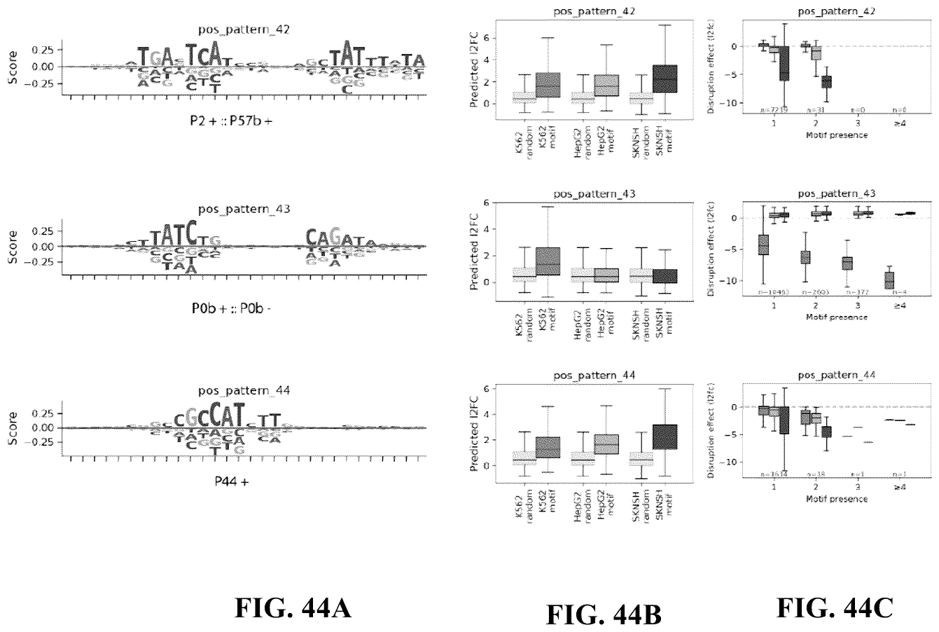

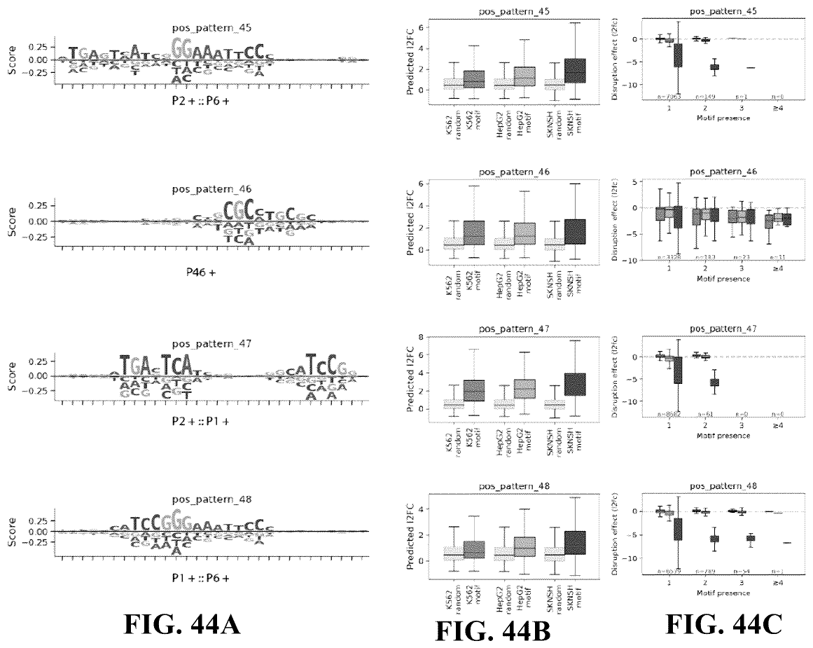

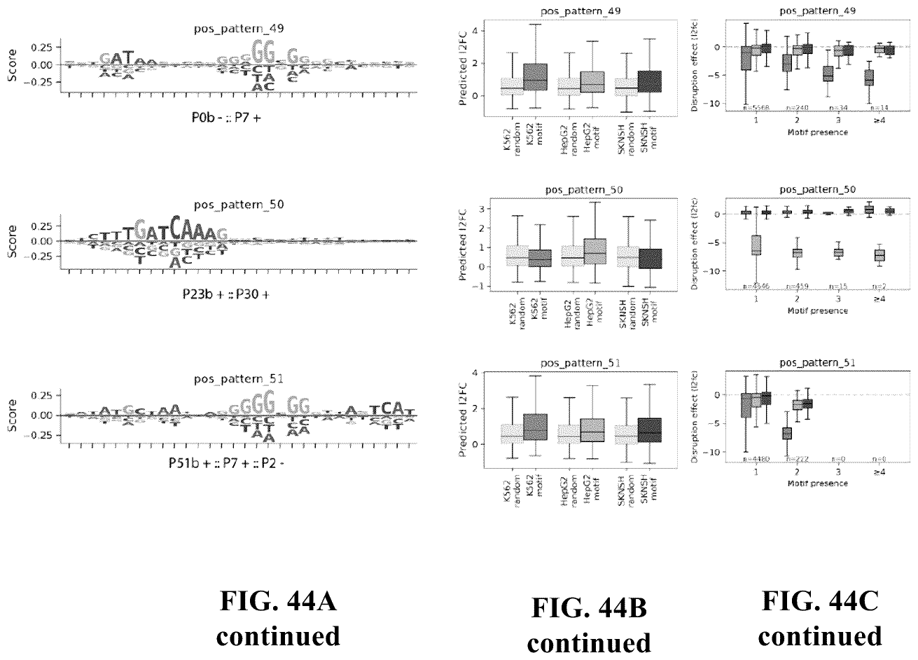

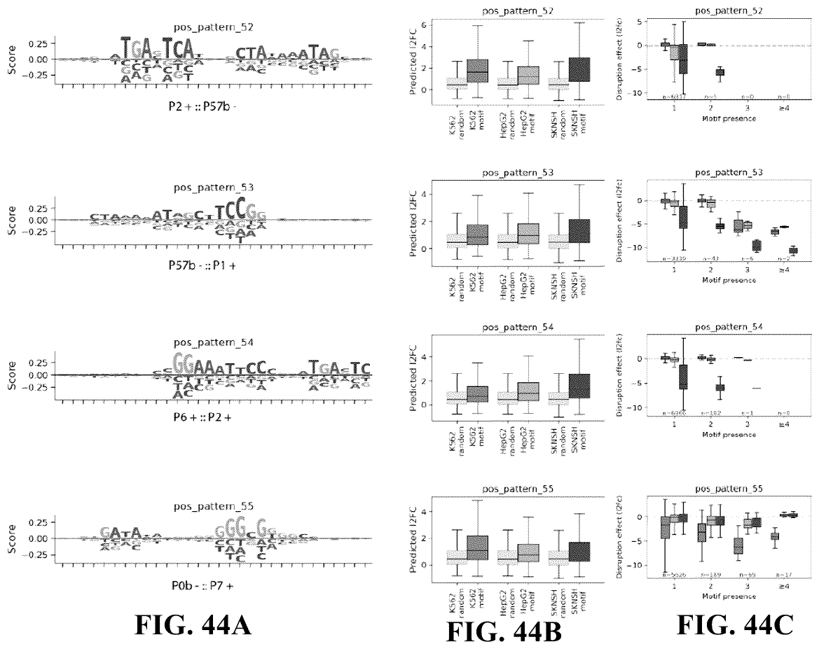

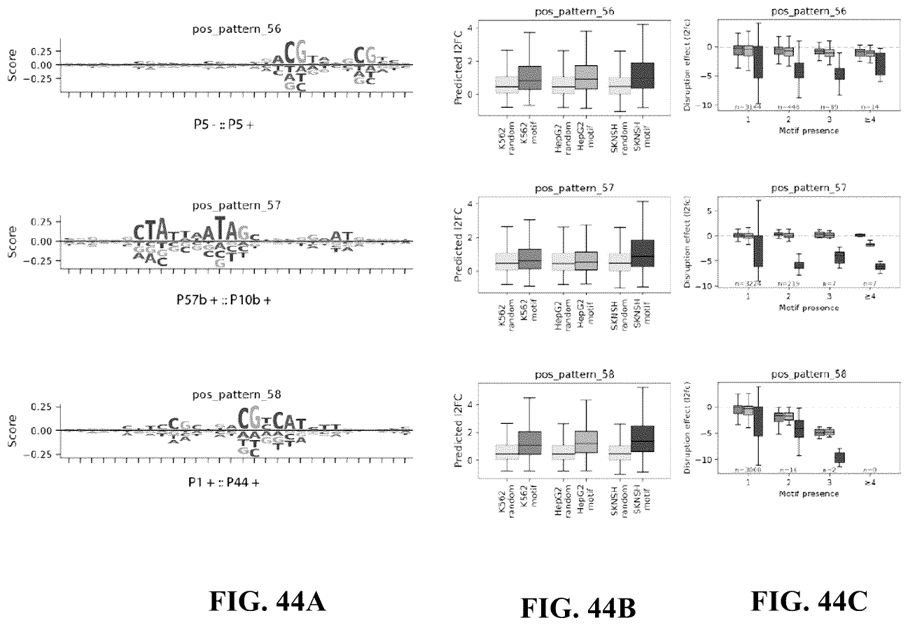

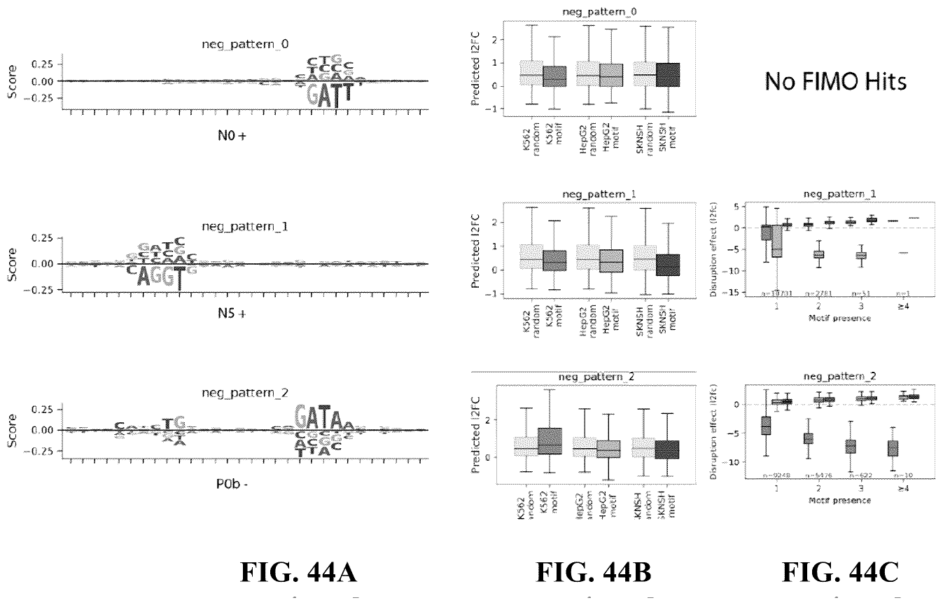

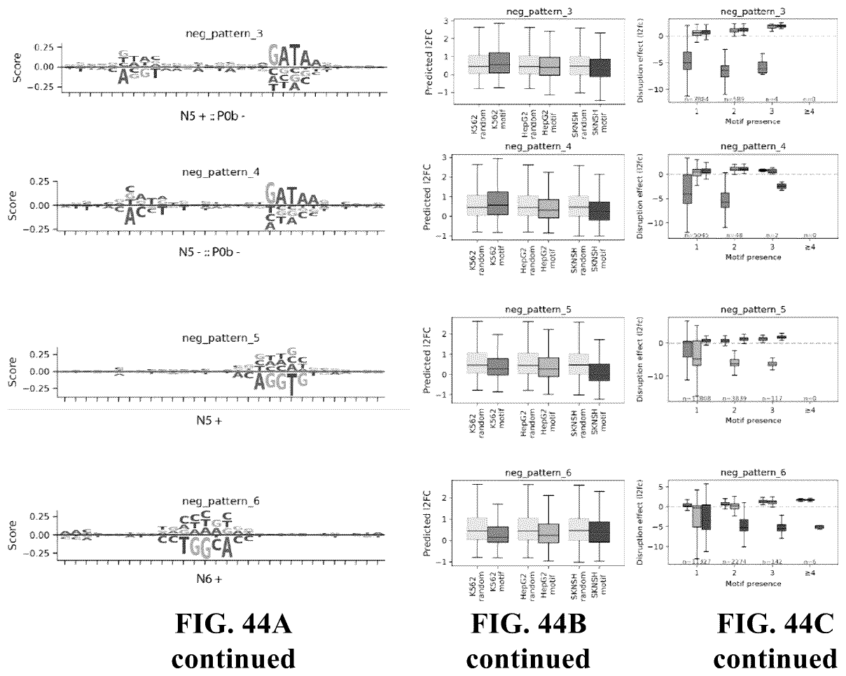

In an embodiment, the engineered CRE contains a motif selected from any motif set forth in FIG. 30A, FIG. 43A, or FIG. 44A. In an embodiment, the engineered CRE contains a motif described in Supplementary Table 7 of Gosi et al. “Machine-guided design of synthetic cell type-specific cis-regulatory elements. Nature. In Review. 2024, which is incorporated by reference as if expressed in its entirety herein).

As used herein, “cell type” refers to the more permanent aspects (e.g., a hepatocyte typically can't on its own turn into a neuron) of a cell's identity. Cell type can be thought of as the permanent characteristic profile or phenotype of a cell. Cell types are often organized in a hierarchical taxonomy, types may be further divided into finer subtypes; such taxonomies are often related to a cell fate map, which reflect key steps in differentiation or other points along a development process. Wagner et al., 2016. Nat Biotechnol. 34 (111): 1145-1160. In an embodiment, the cell type is a diseased or abnormal cell type. As used herein, “cell state” are used to describe transient elements of a cell's identity. Cell state can be thought of as the transient characteristic profile or phenotype of a cell. Cell states arise transiently during time-dependent processes, either in a temporal progression that is unidirectional (e.g., during differentiation, or following an environmental stimulus or disease condition or infection) or in a state vacillation that is not necessarily unidirectional and in which the cell may return to the origin state. Vacillating processes can be oscillatory (e.g., cell-cycle or circadian rhythm) or can transition between states with no predefined order (e.g., due to stochastic, or environmentally controlled, molecular events). These time-dependent processes may occur transiently within a stable cell type (as in a transient environmental response), or may lead to a new, distinct type (as in differentiation). Wagner et al., 2016. Nat Biotechnol. 34 (111): 1145-1160. In an embodiment, the cell state is a disease state.

In this context herein, “specificity” refers to having CRE activity and/or greater CRE activity in one or a few first ith cell types, tissue types, cell states, environments, etc., such as desired cell types, tissue types, cell states, environments, etc. and/or less CRE activity in one or more other second cell types, tissue types, cell states, environments, etc., such as undesired cell types, tissue types, cell states, environments etc. The amount of specific CRE activity in the one or a few first ith cell types, tissue types, cell states, environments, etc. is 0.01-0.1, 0.1-1, 1-100, 100-1,000, 1,000 to 10,000 fold or more greater in the one or a few first ith cell types, tissue types, cell states, environments, etc., as compared to the second cell types, tissue types, cell states, environments, etc., such as undesired cell types, tissue types, cell states, environments etc. In an embodiment, the first ith cell type(s), tissue type(s), cell state(s), environment(s), are those used to generate a MPRA data set of CRE-activity used to train a machine learning network and provides empirical cell (or tissue, or state, or environmental, etc.) specific and non-specific MPRA CRE-activity measurements to a computer implemented model.

As used herein “identified CRE” refers to a CRE that is elucidated by employing the computer implemented model of the present invention to interrogate a nucleic acid input sequence, such as a genome or portion thereof or epigenome or portion thereof, so as to identify sequences in the nucleic acid input sequence with cell type, tissue type, cell state, and/or environment etc., specificity.

As used herein “engineered CRE” refers to a CRE that is designed ab initio by employing the computer implemented model of the present invention so as to generate from an input nucleic acid sequence a nucleic acid sequence having optimized or maximized CRE activity in a specific cell type, tissue type, cell state, environment, etc.

In an embodiment, the identified or engineered CRE is identical to a sequence in a genome. In an embodiment, an engineered CRE does not have a significant match or identity to sequence in a genome of an organism. In an embodiment, an engineered CRE has 0% (meaning no identity) to 50% identity to a sequence in a genome of an organism. In an embodiment an engineered CRE. In an embodiment, even where there is some (i.e., less than 100 percent but greater than 0 percent) identity to a reference genomic sequence, the reference genomic sequence does not have cell type specific, tissue type specific, cell state specific, environment specific, etc. activity, particularly when compared to the engineered CRE. In an embodiment, where the engineered CRE has some identity to a reference genomic sequence the engineered CRE has increased (e.g., 0.01-0.1, 0.1-1, 1-100, 100-1,000, 1,000 to 10,000 fold or more greater) cell type specificity, tissue type specificity, cell state specificity, environment specificity, etc. as compared to the reference genomic sequence. In an embodiment, the reference genome sequence is from a vertebrate or invertebrate. In an embodiment, the reference genome sequence is from a mammal, avian, reptile, fish, or amphibian. In an embodiment, the reference genome sequence is from a human or non-human primate. In an embodiment, the reference genome sequence is from a plant.

In an embodiment, the CRE, such as an engineered CRE, is or contains a polynucleotide as in Supplementary Tables 2 and/or 10 of Gosai et al. “Machine-guided design of synthetic cell cis-regulatory type-specific elements” BioRxiv doi: doi.org/10.1101/2023.08.08.552077 (2023), which are incorporated by reference as if expressed in their entireties herein. In an embodiment, the CRE, such as an engineered CRE, contains a polynucleotide motif as set forth in FIG. 30A, 43A, 44A, and/or described in Supplementary Table 7 of Gosi et al. “Machine-guided design of synthetic cell type-specific cis-regulatory elements. Nature. In Review. 2024, which is incorporated by reference as if expressed in its entirety herein).

In an embodiment, the CREs of the present invention are enhancers. In other words, In an embodiment, the CREs of the present invention have enhancer activity. In an embodiment, the CREs of the present invention are promoters. In other words, In an embodiment, the CREs of the present invention have promoter activity. In an embodiment, the CREs of the present invention are insulators. In other words, In an embodiment, the CREs of the present invention have insulator activity. In an embodiment, the CREs of the present invention are silencers. In other words, In an embodiment, the CREs of the present invention have silencer activity.

In an embodiment the engineered CRE is composed of one or more identified or engineered CREs of the present invention described herein. In an embodiment, the engineered CRE is composed of 2, 3, 4, 5, 6, 7, 8, 9, 10 or more CREs. In such embodiments, the two or more CREs are operatively coupled to each other and/or a nucleic acid that they regulate.

In an embodiment where an engineered CRE contains two or more CREs of the present invention, each of the CREs are the same. In an embodiment where an engineered CRE contains two or more CREs of the present invention, each of the CREs are different. In an embodiment where an engineered CRE contains two or more CREs of the present invention, at least two of the two or more CREs are the same. In an embodiment where an engineered CRE contains two or more CREs of the present invention, at least two of the two or more CREs are different. In an embodiment where an engineered CRE contains two or more CREs of the present invention, the two or more CREs are all enhancers, silencers, insulators, or promoters. In an embodiment where an engineered CRE contains two or more CREs of the present invention, the two or more CREs are each independently selected from an enhancer, a silencer, an insulator, or a promoter. In an embodiment where an engineered CRE contains two or more CREs of the present invention, each of the two or more CREs have a different activity type (e.g., enhancer activity, promoter activity, insulator activity, or silencer activity). In an embodiment where an engineered CRE contains two or more CREs of the present invention, the two or more CREs all have the same activity type. In an embodiment where an engineered CRE contains two or more CREs of the present invention, at least two of the two or more CREs are enhancers, silencers, insulators, or promoters. In an embodiment where an engineered CRE contains two or more CREs of the present invention, at least two of the two or more CREs have a different activity type.

In an embodiment, one or more CREs of the present invention are specifically active in vertebrate cells or invertebrate cells. In an embodiment, one or more CREs of the present invention are specifically active in mammalian, avian, amphibian, or reptile cells. In an embodiment, one or more CREs of the present invention are specifically active in human or non-human primate cells. In an embodiment, one or more CREs of the present invention are specifically active in brain cells, neurons of the central nervous system, neurons of the peripheral nervous system, neuronal support cells (e.g., astrocytes, microglia, dendritic cells, Schwann cells, etc.), blood-brain barrier cells (e.g., endothelial cells, pericytes, astrocytes, microglia), auditory hair cells, supporting cells of the inner ear (e.g., Hensen's cells, Deiter's cells, pillar cells, inner phalangeal cells, and border cells), retinal cells (e.g., rods, cones, retinal ganglion cells, biopolar cells, horizontal cells, and amacrine cells), neuroendocrine cells (e.g., chromophobe cells (including amphophils and melanotrophs)), chromophils (e.g., acidophil cells and basophil cells), Oxyphil cells, pulmonary neuroendocrine cells) parathyroid cells, thyroid cells, pituitary cells, adrenal cells (including, but not limited to, adrenocortical cells, chromaffin cells), kidney cells (e.g., kidney vasculature endothelium cells, glomerular endothelial cells, kidney capillary cells, kidney arteriole and arterial cells, vas afferens cells, vas efference cells, peritubular capillaries, vein and venule cells, ascending vasa recta cells, descending vasa recta cells, mesangial cells, pericytes, kidney smooth muscle cells, kidney juxtaglomerular cells, adult podocytes, podocyte progenitors, proximal convoluted tubule cells, proximal straight tubule cells, proximal tubular progenitors, injured proximal tubular cells, descending loop of Henle cells, ascending thin limb loop of Henle cells, macula densa cells, distal convoluted tubule 1 cells, distal convoluted tubule 2 cells, connecting tubule cells, collecting duct-principal cells, Pan-collecting duct-intercalated cells, collecting duct-intercalated cells (type A), collecting duct-intercalated cells (type B), Collecting duct-transitional cells, immune cells present in the kidney such as macrohpages, neutrophils, basophils, dendritic cells 11b+, dendritic cells 11b−, plasmocytoid dendritic cells, B cells, T cells, CD4 T cells CD8 effector cells, T regulatory cells, Natural Killer T cells, Natural Killer cells (see also, Balzer et al., Annu Rev Physiol. 2022 Feb. 10; 84:507-531), pancreatic cells (e.g., pancreatic islet cells including alpha (produce glucagon), beta (produce insulin and amylin), delta cells (produce somatostatin), gamma cells (produce pancreatic polypeptide), epsilon cells (produce ghrelin) cells; pancreatic acinar cells, and/or pancreatic ductal cells), spleen cells, liver cells (e.g., hepatocytes, hepatic stellate cells, Kupffer cells, and/or liver sinusoidal endothelial cells), cardiac cells (e.g., cardiac fibroblasts, cardiomyocytes, cardiac smooth muscle cells, and cardiac endothelial cells, and/or sinoatrial nodal cells). Intestinal cells (e.g., enterocytes, goblet cells, enteroendocrine cells, Paneth cells, intestinal progenitor cells, intestinal smooth muscle cells, duodenal cells, jejunal cells, ileum cells, and/or colonocytes), hair follicles, skin cells (e.g., basal skin cells, keratinocytes, melanocytes, Langerhans cells, and/or Merkel cells), rectal cells, sweat gland cells (e.g., secretory cells, such as myoepithelial cells and secretory luminal cells, and ductal cells, such as luminal cells and basal cells), lung cells (e.g., epithelial cells, cilia cells, goblet cells, and/or basal cells), bone cells (e.g., osteoblasts, osteocytes, osteoclasts, bone lining cells, and osteogenic cells), periosteum cells, smooth muscle cells, striated muscle cells, tenocytes, ligament fibroblasts, endothelial cells, testicular cells (e.g., germ cells (sperm cells, spermatogonia, spermatids, etc.), Sertoli cells, Leydig cells, peritubular hyoid cells, epidiymal cells, and/or vas deferns cells), prostate cells (e.g., prostate epithelial cells (including luminal secretory cells, basal cells, and neuroendocrine cells) and/or prostate stromal cells (including prostate smooth muscle cells and fibroblasts), bladder cells, urethral cells, uterine cells, oocytes, fallopian tube cells, vaginal cells, cervical cells, blood cells (e.g., erythrocytes), blood progenitor cells, immune cells (e.g., T cells (CD4+ T cells, CD8+ T cells, regulatory T cells, Natural Killer T cells, engineered T cells (e.g., CAR-T cells)), B cells, plasma cells, plasmablasts, natural killer cells, monocytes, macrophages, neutrophils, basophils, eosinophils, dendritic cells, embryonic stem cells, pluripotent stem cells, totipotent stem cells, multipotent stem cells, mesenchymal stem cells, induced pluripotent stem cells, chondrocytes, adipocytes (white and brown adipocytes), stomach cells (including foveolar cells, parietal cells, chief cells, and endocrine/neuroendocrine cells), etc.

In an embodiment, the one or more CREs of the present invention are specifically active in muscle tissue, blood, bone, connective tissue, epithelial tissue, nervous tissue, and/or the like.

In an embodiment, the one or more CREs of the present invention are specifically active in a plant or algal cell. In an embodiment, the one or more CREs of the present invention are specifically active in root cells, stem cells, leaf cells, flower cells, fruit cells, seeds, meristematic cells, parenchyma cells, collenchyma cells, sclerenchyma cells, xylem cells, phloem cells, reproductive cells (e.g., pistal cells, stamen cells) and/or the like.

In an embodiment, the one or more CREs of the present invention are specifically active in a particular cell state. In an embodiment, one or more CREs of the present invention are specifically active in normal, non-diseased cells (i.e., a normal or healthy cell state). In an embodiment, one or more CREs of the present invention are specifically active in abnormal, diseased cells (i.e., a diseased cell state). In an embodiment, the diseased cells are cancer cells, exhausted T cells or exhausted engineered T cells (e.g., CAR-T cells). In an embodiment, the cells exhibit a disease state shown in Table 1.

TABLE 1
DISEASE STATES

| | |
|---|---|
| Disease States | The disease state is an infection (e.g., a fungal infection, a bacterial infection, a |
| | parasite infection, or a viral infection), an organ disease, a blood disease, an |
| | immune system disease, a cancer, a brain and nervous system disease, an endocrine |
| | disease, a pregnancy or childbirth-related disease, an inherited disease, or an |
| | environmentally-acquired disease. |
| Viral Infections | Viral infections and diseases caused by a double-stranded RNA virus, a positive |
| | sense RNA virus, a negative sense RNA virus, a retrovirus, or a combination |
| | thereof, or the viral infection is caused by a Coronaviridae virus, a Picornaviridae |
| | virus, a Caliciviridae virus, a Flaviviridae virus, a Togaviridae virus, a |
| | Bornaviridae, a Filoviridae, a Paramyxoviridae, a Pneumoviridae, a |
| | Rhabdoviridae, an Arenaviridae, a Bunyaviridae, an Orthomyxoviridae, or a |
| | Deltavirus, or the viral infection is caused by Coronavirus, SARS, Poliovirus, |
| | Rhinovirus, Hepatitis A, Norwalk virus, Yellow fever virus, West Nile virus, |
| | Hepatitis C virus, Dengue fever virus, Zika virus, Rubella virus, Ross River virus, |
| | Sindbis virus, Chikungunya virus, Borna disease virus, Ebola virus, Marburg virus, |
| | Measles virus, Mumps virus, Nipah virus, Hendra virus, Newcastle disease virus, |
| | Human respiratory syncytial virus, Rabies virus, Lassa virus, Hantavirus, |
| | Crimean-Congo hemorrhagic fever virus, Influenza, or Hepatitis D virus. |
| Plant Viruses | Disease caused from plant viruses selected from the group comprising Tobacco |
| | mosaic virus (TMV), Tomato spotted wilt virus (TSWV), Cucumber mosaic virus |
| | (CMV), Potato virus Y (PVY), the RT virus Cauliflower mosaic virus (CaMV), |
| | Plum pox virus (PPV), Brome mosaic virus (BMV), Potato virus X (PVX), Citrus |
| | tristeza virus (CTV), Barley yellow dwarf virus (BYDV), Potato leafroll virus |
| | (PLRV), Tomato bushy stunt virus (TBSV), rice tungro spherical virus (RTSV), |
| | rice yellow mottle virus (RYMV), rice hoja blanca virus (RHBV), maize rayado |
| | fino virus (MRFV), maize dwarf mosaic virus (MDMV), sugarcane mosaic virus |
| | (SCMV), Sweet potato feathery mottle virus (SPFMV), sweet potato sunken vein |
| | closterovirus (SPSVV), Grapevine fanleaf virus (GFLV), Grapevine virus A |
| | (GVA), Grapevine virus B (GVB), Grapevine fleck virus (GFkV), Grapevine |
| | leafroll-associated virus-1, -2, and -3, (GLRaV-1, -2, and -3), Arabis mosaic virus |
| | (ArMV), or Rupestris stem pitting-associated virus (RSPaV). |
| DNA Viruses | Diseases caused from DNA viruses from the Family Myoviridae, Podoviridae, |
| | Siphoviridae, Alloherpesviridae, Herpesviridae (including human herpes virus, |
| | and Varicella Zozter virus), Malocoherpesviridae, Lipothrixviridae, Rudiviridae, |
| | Adenoviridae, Ampullaviridae, Ascoviridae, Asfarviridae (including African |
| | swine fever virus), Baculoviridae, Cicaudaviridae, Clavaviridae, Corticoviridae, |
| | Fuselloviridae, Globuloviridae, Guttaviridae, Hytrosaviridae, Iridoviridae, |
| | Maseilleviridae, Mimiviradae, Nudiviridae, Nimaviridae, Pandoraviridae, |
| | Papillomaviridae, Phycodnaviridae, Plasmaviridae, Polydnaviruses, |
| | Polyomaviridae (including Simian virus 40, JC virus, BK virus), Poxviridae |
| | (including Cowpox and smallpox), Sphaerolipoviridae, Tectiviridae, Turriviridae, |
| | Dinodnavirus, Salterprovirus, Rhizidovirus, among others. |
| Retroviruses | Diseases caused by retroviruses that include one or more of, or any combination |
| | of, viruses of the Genus Alpharetrovirus, Betaretrovirus, Gammaretrovirus, |
| | Deltaretrovirus, Epsilonretrovirus, Lentivirus, Spumavirus, or the Family |
| | Metaviridae, Pseudoviridae, and Retroviridae (including HIV), Hepadnaviridae |
| | (including Hepatitis B virus), and Caulimoviridae (including Cauliflower mosaic |
| | virus). |
| Pathogenic | Diseases caused from pathogenic bacteria, including, but not limited to, |
| Bacteria | Acinetobacter baumanii, Actinobacillus sp., Actinomycetes, Actinomyces sp. (such |
| | as Actinomyces israelii and Actinomyces naeslundii), Aeromonas sp. (such as |
| | Aeromonas hydrophila, Aeromonas veronii biovar sobria (Aeromonas sobria), |
| | and Aeromonas caviae), Anaplasma phagocytophilum, Anaplasma marginal, e |
| | Alcaligenes xylosoxidans, Acinetobacter baumanii, Actinobacillus |
| | actinomycetemcomitans, Bacillus sp. (such as Bacillus anthracis, Bacillus cereus, |
| | Bacillus subtilis, Bacillus thuringiensis, and Bacillus stearothermophilus), |
| | Bacteroides sp. (such as Bacteroides fragilis), Bartonella sp. (such as Bartonella |
| | bacilliformis and Bartonella henselae, Bifidobacterium sp., Bordetella sp. (such |
| | as Bordetella pertussis, Bordetella parapertussis, and Bordetella bronchiseptica), |
| | Borrelia sp. (such as Borrelia recurrentis, and Borrelia burgdorferi), Brucella sp. |
| | (such as Brucella abortus, Brucella canis, Brucella melintensis and Brucella suis), |
| | Burkholderia sp. (such as Burkholderia pseudomallei and Burkholderia cepacia), |
| | Campylobacter sp. (such as Campylobacter jejuni, Campylobacter coli, |
| | Campylobacter lari and Campylobacter fetus), Capnocytophaga sp., |
| | Cardiobacterium hominis, Chlamydia trachomatis, Chlamydophila pneumoniae, |
| | Chlamydophila psittaci, Citrobacter sp. Coxiella burnetii, Corynebacterium sp. |
| | (such as, Corynebacterium diphtheriae, Corynebacterium jeikeum and |
| | Corynebacterium), Clostridium sp. (such as Clostridium perfringens, Clostridium |
| | difficile, Clostridium botulinum and Clostridium tetani), Eikenella corrodens, |
| | Enterobacter sp. (such as Enterobacter aerogenes, Enterobacter agglomerans, |
| | Enterobacter cloacae and Escherichia coli, including opportunistic Escherichia |
| | coli, such as enterotoxigenic E. coli, enteroinvasive E. coli, enteropathogenic E. |
| | coli, enterohemorrhagic E. coli, enteroaggregative E. coli and uropathogenic E. |
| | coli) Enterococcus sp. (such as Enterococcus faecalis and Enterococcus faecium) |
| | Ehrlichia sp. (such as Ehrlichia chafeensia and Ehrlichia canis), Erysipelothrix |
| | rhusiopathiae, Eubacterium sp., Francisella tularensis, Fusobacterium |
| | nucleatum, Gardnerella vaginalis, Gemella morbillorum, Haemophilus sp. (such |
| | as Haemophilus influenzae, Haemophilus ducreyi, Haemophilus aegyptius, |
| | Haemophilus parainfluenzae, Haemophilus haemolyticus and Haemophilus |
| | parahaemolyticus, Helicobacter sp. (such as Helicobacter pylori, Helicobacter |
| | cinaedi and Helicobacter fennelliae), Kingella kingii, Klebsiella sp. (such as |
| | Klebsiella pneumoniae, Klebsiella granulomatis and Klebsiella oxytoca), |
| | Lactobacillus sp., Listeria monocytogenes, Leptospira interrogans, Legionella |
| | pneumophila, Leptospira interrogans, Peptostreptococcus sp., Mannheimia |
| | hemolytica, Moraxella catarrhalis, Morganella sp., Mobiluncus sp., Micrococcus |
| | sp., Mycobacterium sp. (such as Mycobacterium leprae, Mycobacterium |
| | tuberculosis, Mycobacterium paratuberculosis, Mycobacterium intracellulare, |
| | Mycobacterium avium, Mycobacterium bovis, and Mycobacterium marinum), |
| | Mycoplasm sp. (such as Mycoplasma pneumoniae, Mycoplasma hominis, and |
| | Mycoplasma genitalium), Nocardia sp. (such as Nocardia asteroides, Nocardia |
| | cyriacigeorgica and Nocardia brasiliensis), Neisseria sp. (such as Neisseria |
| | gonorrhoeae and Neisseria meningitidis), Pasteurella multocida, Plesiomonas |
| | shigelloides. Prevotella sp., Porphyromonas sp., Prevotella melaninogenica, |
| | Proteus sp. (such as Proteus vulgaris and Proteus mirabilis), Providencia sp. |
| | (such as Providencia alcalifaciens, Providencia rettgeri and Providencia stuartii), |
| | Pseudomonas aeruginosa, Propionibacterium acnes, Rhodococcus equi, |
| | Rickettsia sp. (such as Rickettsia rickettsii, Rickettsia akari and Rickettsia |
| | prowazekii, Orientia tsutsugamushi (formerly: Rickettsia tsutsugamushi) and |
| | Rickettsia typhi), Rhodococcus sp., Serratia marcescens, Stenotrophomonas |
| | maltophilia, Salmonella sp. (such as Salmonella enterica, Salmonella typhi, |
| | Salmonella paratyphi, Salmonella enteritidis, Salmonella cholerasuis and |
| | Salmonella typhimurium), Serratia sp. (such as Serratia marcesans and Serratia |
| | liquifaciens), Shigella sp. (such as Shigella dysenteriae, Shigella flexneri, Shigella |
| | boydii and Shigella sonnei), Staphylococcus sp. (such as Staphylococcus aureus, |
| | Staphylococcus epidermidis, Staphylococcus hemolyticus, Staphylococcus |
| | saprophyticus), Streptococcus sp. (such as Streptococcus pneumoniae (for |
| | example chloramphenicol-resistant serotype 4 Streptococcus pneumoniae, |
| | spectinomycin-resistant serotype 6B Streptococcus pneumoniae, streptomycin- |
| | resistant serotype 9V Streptococcus pneumoniae, erythromycin-resistant serotype |
| | 14 Streptococcus pneumoniae, optochin-resistant serotype 14 Streptococcus |
| | pneumoniae, rifampicin-resistant serotype 18C Streptococcus pneumoniae, |
| | tetracycline-resistant serotype 19F Streptococcus pneumoniae, penicillin- |
| | resistant serotype 19F Streptococcus pneumoniae, and trimethoprim-resistant |
| | serotype 23F Streptococcus pneumoniae, chloramphenicol-resistant serotype 4 |
| | Streptococcus pneumoniae, spectinomycin-resistant serotype 6B Streptococcus |
| | pneumoniae, streptomycin-resistant serotype 9V Streptococcus pneumoniae, |
| | optochin-resistant serotype 14 Streptococcus pneumoniae, rifampicin-resistant |
| | serotype 18C Streptococcus pneumoniae, penicillin-resistant serotype 19F |
| | Streptococcus pneumoniae, or trimethoprim-resistant serotype 23F Streptococcus |
| | pneumoniae), Streptococcus agalactiae, Streptococcus mutans, Streptococcus |
| | pyogenes, Group A streptococci, Streptococcus pyogenes, Group B streptococci, |
| | Streptococcus agalactiae, Group C streptococci, Streptococcus anginosus, |
| | Streptococcus equismilis, Group D streptococci, Streptococcus bovis, Group F |
| | streptococci, and Streptococcus anginosus Group G streptococci), Spirillum |
| | minus, Streptobacillus moniliformi, Treponema sp. (such as Treponema carateum, |
| | Treponema petenue, Treponema pallidum and Treponema endemicum, |
| | Tropheryma whippelii, Ureaplasma urealyticum, Veillonella sp., Vibrio sp. (such |
| | as Vibrio cholerae, Vibrio parahemolyticus, Vibrio vulnificus, Vibrio |
| | parahaemolyticus, Vibrio vulnificus, Vibrio alginolyticus, Vibrio mimicus, Vibrio |
| | hollisae, Vibrio fluvialis, Vibrio metchnikovii, Vibrio damsela and Vibrio furnisii), |
| | Yersinia sp. (such as Yersinia enterocolitica, Yersinia pestis, and Yersinia |
| | pseudotuberculosis) and Xanthomonas maltophilia among others. |
| Fungal Microbes | Diseases caused by Aspergillus, Blastomyces, Candidiasis, Coccidiodomycosis, |
| | Cryptococcus neoformans, Cryptococcus gatti, Histoplasma, Mucroymcosis, |
| | Pneumocystis, Sporothrix, fungal eye infections, ringworm, Exserohilum, and |
| | Cladosporium. |
| Fungal Yeasts and | Diseases caused by Aspergillus species, a Geotrichum species, a Saccharomyces |
| Molds | species, a Hansenula species, a Candida species, a Kluyveromyces species, a |
| | Debaryomyces species, a Pichia species, or combination thereof. Example molds |
| | include, but are not limited to, a Penicillium species, a Cladosporium species, a |
| | Byssochlamys species, or a combination thereof. |
| Infectious Disease | Acne- Proprionibacterium acnes |
| Names and Their | Acute bacterial rhinosinusitis- most common = Streptococcus |
| Etiologies | pneumoniae (G+ coccus) and Haemophilus influenzae (G− pleomorphic |
| (A) | rod) |
| | Acute hemorrhagic conjunctivitis (*) - Coxsackie A-24 virus |
| | (Picornavirus: Enterovirus), Enterovirus 70 (Picornavirus: Enterovirus) |
| | Acute hemorrhagic cystitis (*) - Adenovirus 11 and 21 (Adenovirus) |
| | Acute rhinosinusitis- respiratory viruses usually |
| | Acquired Immunodeficiency Sydrome (AIDS) - Human |
| | Immunodeficiency Virus (HIV-1 and HIV-2) (retrovirus) |
| | Acrodermatitis chronica atrophicans (ACA)- late skin manifestation of |
| | latent Lyme disease- Borrelia burgdorferi (Spirochetes) |
| | Adult T-cell Leukemia-Lymphoma (ATLL) - Human T-cell Leukemia |
| | viruses I or II (retrovirus) |
| | African Sleeping Sickness - Trypanosomiasis - African = Trypanosoma |
| | brucei rhodesiense, Trypanosoma brucei gambiense (tsetse fly-borne) |
| | AIDS- Human immunodeficiency virus (HIV) |
| | Alveolar hydatid - Echinococcus multilocularis (larval cestode infection) |
| | Amebiasis - Entamoeba histolytica (protozoan parasite) |
| | Amebic meningoencephalitis- Naegleria fowleri, Acanthamoeba species, |
| | and Balamuthia mandrillaris (protozoan) |
| | Anthrax - Black Bane- Malignant pustule- Wool sorter's disease- |
| | Tanner's disease- Bacillus anthracis (G+ rod: sporulating: aerobic) |
| | Ascariasis - Roundworm infections - Ascaris lumbricoides (intestinal |
| | nematode) |
| | Aseptic meningitis (*)- Coxsackie B virus, Echovirus, Mumps virus, |
| | Coxsackie A virus, Polio virus, (5 most common) then Human |
| | Herpesvirus 1, Arboviruses, Lymphocytic choriomeningitis viruses |
| | (Arenavirus), Encephalomycarditis viruses, Louping Ill virus, |
| | Pseudolymphocytic meningitis virus, Hepatitis viruses, Adenoviruses, |
| | Rhinoviruses. |
| | Athlete's foot - Tinea pedis - Trichophyton spp., and Epidermophyton |
| | floccosum (fungi) |
| | Australian tick typhus- Australian Spotted Fever- Queensland Tick |
| | Typhus- Rickettsia australis, (G−; intracellular bacteria) |
| | Avian Influenza- Bird Flu- Influenza virus A H5N1 |
| (B) | Babesiosis - Babesia microti (protozoan parasite; transmitted by deer |
| | tick) |
| | Bacillary angiomatosis - Bartonella henselae (pleomorphic G−) |
| | Bacterial meningitis- Streptococcus agalactiae, Escherichia coli, |
| | Streptococcus pneumoniae, Neisseria meningitidis, Listeria |
| | monocytogenes, Gram negative rod-shaped bacteria |
| | Bacterial vaginosis- Gardnerella vaginalis, Mycoplasma hominis and |
| | various anaerobic bacteria including Mobiluncus sp., and Prevotella sp. |
| | Balanitis- Candida albicans (yeast)- most common. |
| | Balantidiasis- Balantidium coli (flagellated protozoan) |
| | Bang's disease - Brucellosis - Brucella sp. (G− coccobacillus; zoonoses) |
| | Bartonellosis - Verruga peruana- Carrion's disease - Oroya fever - |
| | Bartonella bacilliformis (weak G− polymorphic) sandfly bites at |
| | elevations of 600 to 2800 meter in Peru, Ecuador and Colombia. |
| | Bay sore - Chiclero's ulcer - Leishmania leishmania mexicana |
| | (protozoan parasite) sandfly |
| | Baylisascaris infection - Racoon roundworm infection- Baylisascaris |
| | procyonis |
| | Beaver fever - giardiasis - Giardia lamblia |
| | Beef tapeworm - Taenia saginata |
| | Bejel - endemic syphilis - Treponema pallidum var. endemicum |
| | Biphasic meningoencephalitis- Central European tick-borne encephalitis- |
| | Czechoslovak tick-borne encephalitis- Diphasic milk fever- Tick-borne |
| | encephalitis- Viral meningoencephalitis- Tick-borne encephalitis virus- |
| | Flaviviridae |
| | Bird Flu- Avian Influenza- Influenza virus A H5N1 |
| | Black Bane- Anthrax- Malignant pustule- Wool sorter's disease- Tanner's |
| | disease- Bacillus anthracis (G+ rod: sporulating: aerobic) |
| | “Black death” (plague) - Yersinia pestis (G− rod: facultative-straight: |
| | zoonoses) |
| | Black piedra- Piedraia hortai (fungal infection of hair shaft) |
| | Blackwater Fever- Malaria- Plasmodium falciparum (sporozoan parasite) |
| | Blastomycosis- Chicago disease- Gilchrist's disease- North American |
| | blastomycosis- Blastomyces dermatitidis (dimorphic fungus) |
| | Blennorrhea of the newborn- Chlamydia trachomatis |
| | Blepharitis- infestation of the eyelash follicle by a mite. This results in an |
| | allergic reaction which leads to an inflammatory reaction and secondary |
| | infection with Staphylococcus aureus or Staphylococcus epidermidis. |
| | Boils - Staphylococcus aureus (G+ coccus) |
| | Bornholm disease (pleurodynia) - Coxsackie B (Picornavirus: |
| | Enterovirus) |
| | Borrelia miyamotoi Disease- Borrelia miyamotoi (G− bacterium; |
| | spirochete) |
| | Botulism - Clostridium botulinum (G+ rod: sporulating: anaerobic) |
| | Boutonneuse fever- Fievre boutonneuse- Tick typhus- Rickettsia conori |
| | (G− intracellular; tick-borne) |
| | Brazilian purpuric fever - Haemophilus aegyptius (G− rod: facultative- |
| | straight: respiratory pathogens) |
| | Break Bone fever- dandy fever- Dengue virus (Flaviviridae) |
| | Brill-Zinsser disease - recrudescent typhus - Rickettsia prowazekii (G− |
| | intracellular; flea-borne) |
| | Bronchitis- Respiratory syncytial virus (Paramyxovirus), Parainfluenza |
| | virus (Paramyxovirus), Influenza virus |
| | Bronchiolitis (*) - Respiratory syncytial virus (Paramyxovirus), |
| | Parainfluenza virus (Paramyxovirus) |
| | Brucellosis - Brucella sp. (G− coccobacillus; zoonoses) |
| | Bubonic plague- Yersinia pestis |
| | Bullous impetigo- Staphylococcus aureus |
| | Buruli ulcers- Mycoburuli ulcers- Mycobacterium ulcerans |
| | Busse-Buschke disease- Cryptococcosis- Torulosis- European |
| | blastomycosis- Cryptococcus neoformans (encapsulated yeast) |
| (C) | California group encephalitis - California encephalitis virus, La Crosse |
| | virus, Jamestown Canyon, Snowshoe hare virus (Bunyavirus) |
| | mosquitoes |
| | Candidiasis- Candidosis- Moniliasis- infection of the mucous membranes |
| | (mouth, esophagus, vagina) caused by the yeast Candida albicans. |
| | Candidosis- Candidiasis- Moniliasis- infection of the mucous membranes |
| | (mouth, esophagus, vagina) caused by the yeast Candida albicans. |
| | Canefield fever- canicola fever- 7-day fever- Weil's disease - |
| | leptospirosis - nanukayami fever- Leptospira interrogans (spiral shaped |
| | bacteria) |
| | Canicola fever- 7-day fever- Weil's disease - leptospirosis - canefield |
| | fever- nanukayami fever- Leptospira interrogans (spiral shaped bacteria) |
| | Capillariasis - Capillaria philippinensis (intestinal nematode) |
| | Carate - Mal del pinto - Pinta - Treponema pallidum var. carateum |
| | Carbuncle - Staphylococcus aureus (G+ coccus) |
| | Carrion's disease - Bartonellosis - Oroya fever - Bartonella bacilliformis |
| | (weak G− polymorphic) sandfly bites at elevations of 600 to 2800 meter |
| | in Peru, Ecuador and Colombia. |
| | Cat Scratch fever - Cat Scratch Disease- Bartonella henselae |
| | (pleomorphic G−) |
| | Cave disease- Darling's Disease- spelunker's disease- Histoplasmosis- |
| | Histoplasma capsulatum (dimorphic fungus) |
| | Central Asian hemorrhagic fever- Congo-Crimean hemorrhagic fever- |
| | Crimean-Congo hemorrhagic fever- Congo fever- Crimean-Congo |
| | hemorrhagic fever virus- Bunyavirus- Nairovirus |
| | Central European tick-borne encephalitis- Diphasic milk fever- Biphasic |
| | meningoencephalitis, Czechoslovak tick-borne encephalitis, Tick-borne |
| | encephalitis, Viral meningoencephalitis, Tick-borne encephalitis virus- |
| | Flaviviridae |
| | Cervical cancer - human papilloma virus (Papovavirus) |
| | Chancroid - Haemophilus ducreyi (G− rod: facultative-straight: |
| | respiratory pathogens) |
| | Chicago disease- Blastomycosis- Gilchrist's disease- North American |
| | blastomycosis- Blastomyces dermatitidis (dimorphic fungus) |
| | Chikungunya fever- Chikungunya virus- Togaviridae- Alphavirus |
| | Chagas disease - Trypanosomiasis - American = Trypanosoma cruzi |
| | (Triatomine bugs = kissing bug or assassin bugs) |
| | Chickenpox - Varicella-Zoster virus (VZV or Human herpes 3 virus) |
| | Chiclero's ulcer - Bay sore - Leishmania leishmania mexicana |
| | (protozoan parasite) sandfly |
| | Chlamydia - Chlamydiae trachomatis (Obligate intracellular) |
| | Chlamydial infection- Chlamydiae trachomatis (Obligate intracellular) |
| | Cholera - Vibrio cholerae (G− rods: facultative-curved: enteric |
| | pathogens) |
| | Chromoblastomycosis - Fonsecaea pedrosoi (fungus) |
| | Clap - Gonorrhea - Neisseria gonorrhoeae (G− cocci) |
| | Clonorchiasis - Liver fluke infection - Clonorchis sinensis (liver flukes) |
| | Coccidioidomycosis- San Joaquin Valley fever, desert rheumatism, |
| | Posada-Wernicke disease- Coccidioides immitis (dimorphic fungus). |
| | Coenurosis - Taenia spp. (larval cestode infection) |
| | Colorado tick fever - Colorado tick fever virus (Reovirus) |
| | Congo fever- Congo-Crimean hemorrhagic fever- Crimean-Congo |
| | hemorrhagic fever- Crimean-Congo hemorrhagic fever virus- Central |
| | Asian hemorrhagic fever- Bunyavirus- Nairovirus |
| | Congo hemorrhagic fever virus- Congo-Crimean hemorrhagic fever- |
| | Crimean- Congo fever- Crimean-Central Asian hemorrhagic fever- |
| | Bunyavirus- Nairovirus |
| | Congo-Crimean hemorrhagic fever- Crimean-Congo hemorrhagic fever- |
| | Congo fever- Crimean-Congo hemorrhagic fever virus- Central Asian |
| | hemorrhagic fever- Bunyavirus- Nairovirus |
| | Condyloma accuminata - Warts - Papilloma virus |
| | Condyloma lata - Treponema pallidum subsp. pallidum (spirochete) |
| | secondary syphilis |
| | Conjunctivitis (*) - Haemophilus aegyptius (G− rod: facultative-straight: |
| | respiratory pathogens), Chlamydiae trachomatis (Obligate intracellular) |
| | Cowpox - vaccinia virus (Poxvirus) |
| | Crabs - Pediculosis - lice |
| | Creutzfeldt-Jakob disease - prion (a protein) |
| | Crimean-Congo hemorrhagic fever- Congo fever- Congo-Crimean |
| | hemorrhagic fever- Crimean-Congo hemorrhagic fever virus- Central |
| | Asian hemorrhagic fever- Bunyavirus- Nairovirus |
| | Croup, infectious - parainfluenza viruses 1-3 (Paramyxovirus) |
| | Cryptococcosis- Busse-Buschke disease- Torulosis- European |
| | blastomycosis- Cryptococcus neoformans (encapsulated yeast) |
| | Cutaneous Larval Migrans - Ancylostoma braziliense (filariform larvae; |
| | parasite) and many other parasitic worms normally found in animals. |
| | Cyclosporiasis- Cyclospora cayetanensis |
| | Cysticercosis - Taenia solium (larval form of the cestode) |
| | Cystic hydatid - Echinococcus granulosus (larval cestode infection) |
| | Cystitis(*) - most common = Escherichia coli, others include Klebsiella |
| | sp, Enterobacter sp., Serratia sp., Proteus sp., Providencia sp., |
| | Morganella sp., Pseudomonas aeruginosa, (the previous organisms are |
| | G− rods), Staphylococcus saprophyticus, Enterococcus sp., |
| | Staphylococcus aureus, Staphylococcus epidermidis, Streptococcus |
| | agalactiae, (G+ cocci), and Candida albicans (yeast) |
| | Czechoslovak tick-borne encephalitis, - Central European tick-borne |
| | encephalitis- Diphasic milk fever- Biphasic meningoencephalitis, Tick- |
| | borne encephalitis, Viral meningoencephalitis, Tick-borne encephalitis |
| | virus- Flaviviridae |
| (D) | Dacryocytitis- Staphylococcus aureus, Staphylococcus epidermidis, |
| | Streptococcus pneumoniae |
| | Dandy fever- Break Bone fever- Dengue virus (Flaviviridae) |
| | Darling's Disease- cave disease- spelunker's disease- Histoplasmosis- |
| | Histoplasma capsulatum (dimorphic fungus) |
| | Deer fly fever, tularemia, lemming fever, rabbit fever, O'Hara disease, |
| | Francis disease, Francisella tularensis (G− rods: facultative-straight: |
| | zoonoses) |
| | Dengue - Break Bone fever- dengue fever - dengue virus (Flavivirus) |
| | Desert rheumatism- Coccidioidomycosis- San Joaquin Valley fever- |
| | Posada-Wernicke disease- Coccidioides immitis (dimorphic fungus). |
| | “Devil's grip”(pleurodynia) - Coxsackie B (Picornavirus: Enterovirus) |
| | Diphasic milk fever- Biphasic meningoencephalitis, Central European |
| | tick-borne encephalitis, Czechoslovak tick-borne encephalitis, Tick- |
| | borne encephalitis, Viral meningoencephalitis, Tick-borne encephalitis |
| | virus- Flaviviridae |
| | Diphtheria - Corynebacterium diphtheriae (G+ rod: non-sporulating: |
| | non-filamentous) |
| | Disseminated Intravascular Coagulation(*) - most commonly |
| | Escherichia coli (G− rod) |
| | Dwarf tapeworm - Hymenolepis nana (intestinal cestode) |
| | Dog tapeworm - Diphylidium caninum (intestinal cestode) |
| | Donovanosis - Granuloma inguinale- Klebsiella granulomatis (G− rod; |
| | Donovan bodies) |
| | Dracontiasis - Guinea Worm - Dirofilaria medinensis (parasitic worm) |
| | Dracunculosis- Dracunculus medinensis (parasite; nematode; “Little |
| | dragon of Medina”) |
| | Duke's disease- viral rash- Coxsackievirus or Echovirus |
| | Dum Dum Disease - Kala Azar - Visceral Leishmaniasis - Leishmania |
| | leishmania donovani, L. leishmania infantum, L. leishmania chagasi |
| | (protozoan parasite) sandfly |
| | Durand-Nicholas-Favre disease - Lymphogranuloma venereum (LGV) - |
| | Chlamydia trachomatis (intracellular G− bacteria; the L serotypes) |
| (E) | Eastern equine encephalitis - EEE virus (Togavirus) |
| | Ebola hemorrhagic fever - Ebola virus (Filovirus) |
| | Ectothrix - fungal infection of the hair shaft - Microsporum, |
| | Trichophyton, and Epidermophyton (fungi) |
| | Ehrlichiosis - Ehrlichia sp. (G− intracellular bacteria) transmitted by ticks |
| | Epidemic typhus- Rickettsia prowazekii, (G− intracellular; spread by lice) |
| | Encephalitis- Mumpsvirus, Human Herpesvirus 1 (Herpes Simplex 1 |
| | Virus), Any of 350 different arboviruses, Enteroviruses (polio, |
| | Coxsackie, ECHO), Adenovirus, Human Immunodeficiency Virus |
| | Endemic Relapsing fever- Borrelia sp. |
| | Endemic syphilis -Bejel - Treponema pallidum var. endemicum |
| | Endophthalmitis- Staphylococcus aureus, Staphylococcus epidermidis, |
| | Bacillus cereus, Streptococcus pneumoniae, Streptococcus pyogenes. |
| | Endothrix - fungal infection of the hair shaft - Microsporum, |
| | Trichophyton, and Epidermophyton (fungi) |
| | Enterobiasis - Pinworm infection - Enterobius vermicularis (intestinal |
| | nematode) |
| | Epidemic Relapsing fever- Borrelia recurrentis |
| | Epiglottitis (*)- Haemophilus influenzae (G− rod: facultative-straight: |
| | respiratory pathogens |
| | Erysipeloid - Erysipelothricosis - Erysipelothrix rhusiopathiae (G+ rod) |
| | Erysipelis- Streptococcus pyogenes |
| | Erythema chronicum migrans - seen in Lyme disease |
| | Erythema marginatum - seen in rheumatic fever |
| | Erythema multiforme - seen in coccidioidomycosis (Coccidioides |
| | immitis) |
| | Erythema nodosum - seen in coccidioidomycosis (Coccidioides immitis) |
| | Erythema nodosum leprosum - Mycobacterium leprae |
| | Erythema infectiosum - (Slapped cheek syndrome; fifth disease) |
| | Parvovirus B19 (Parvovirus) |
| | Erythrasma - Corynebacterium minutissimum |
| | Espundia - Leishmania viannia braziliensis (protozoan parasite) sandfly |
| | Eumycotic mycetoma- Madura foot- Pseudallescheria boydii, Madurella |
| | grisea, Madurella mycetomatis (fungi) |
| | European blastomycosis- Torulosis- Busse-Buschke disease- |
| | Cryptococcosis- Cryptococcus neoformans (encapsulated yeast) |
| | Eyeworm - Loiasis - Loa loa (parasitic worm) |
| | Exanthem subitum - Roseola infantum - Sixth disease - Zahorsky's |
| | disease- “Sudden Rash”, Rose rash of infants, 3-day fever- Human |
| | Herpes virus 6 (HHV-6) |
| (F) | Far Eastern tick-borne encephalitis- Spring-summer encephalitis- |
| | Russian spring-summer encephalitis- Taiga encephalitis- Russian spring- |
| | summer encephalitis virus- Flaviviridae |
| | Fascioliasis - Liver fluke infection - Fasciola hepatica (liver flukes) |
| | Fievre boutonneuse- Tick typhus- Rickettsia conori |
| | “Fifth” disease (erythema infectiosum) - Parvovirus B19 (Parvovirus) |
| | Filatow-Dukes' Disease- Scalded Skin Syndrome- Ritter's Disease- |
| | Staphylococcus aureus- (exfoliative toxin producing strains) |
| | Fish tapeworm - Diphyllobothrium latum |
| | Fitz-Hugh-Curtis syndrome - Perihepatitis - Neisseria gonorrhoeae (G− |
| | cocci) |
| | Five-day fever, Trench fever, Shinbone fever, Wolhynia fever, Quintana |
| | fever, His-Werner disease- Bartonella quintana (G− rod) |
| | Flinders Island Spotted Fever- Rickettsia honei |
| | Flu- Influenza - Influenza viruses A, B, and C (Orthomyxovirus) |
| | Four Corners Disease - Human Pulmonary Syndrome (HPS) - Sin |
| | Nombre Virus (Hantaan virus group; Bunyavirus) |
| | 14-day measles- Rubeola-measles- Morbilli- Hard measles- Rubeola |
| | virus |
| | Frambesia - Yaws -Treponema pallidum var. pertenue |
| | Francis disease, O'Hara disease, deer fly fever, lemming fever, tularemia, |
| | rabbit fever, Francisella tularensis (G− rods: facultative-straight: |
| | zoonoses) |
| | Furunculosis = boil- furuncle- Staphylococcus aureus (G+ coccus) |
| | Folliculitis - Staphylococcus aureus (G+ coccus) |
| (G) | Gas gangrene - Clostridium perfringens (G+ rod: sporulating: anaerobic) |
| | Gastroenteritis - Norwalk virus (Calicivirus), rotavirus (Reovirus) |
| | Genital Herpes- Herpes Simplex Virus-2 (Human Herpes Virus-2) |
| | occasionally HSV-1 (HHV-1) |
| | Genital Warts- Human Papilloma virus (various serotypes) |
| | German measles- Rubella- 3-day measles- Rubella virus |
| | Gerstmann-Straussler-Scheinker (GSS) - - prion (a protein) |
| | Giardiasis - Giardia lamblia |
| | Gilchrist's disease- Chicago disease- Blastomycosis- North American |
| | blastomycosis- Blastomyces dermatitidis (dimorphic fungus) |
| | Gingivostomatitis - HSV-1 (Herpesvirus) |
| | Gingivitis- various anaerobic bacteria in the mouth |
| | Glanders - Burkholderia mallei (used to be named Pseudomonas mallei; |
| | G− rod) |
| | Gnathostomiasis- Gnathostoma spinigerum (third stage larvae of a |
| | nematode (parasitic worm)) |
| | Gonorrhea - Neisseria gonorrhoeae (G− cocci) |
| | Granuloma inguinale - Donovanosis- Klebsiella granulomatis (G− rod) |
| | Guinea Worm - Dracontiasis - Dirofilaria medinensis (parasitic worm) |
| (H) | Hamburger disease- Hemolytic Uremic Syndrome- Escherichia coli |
| | O157 H7 strain. |
| | Hand-foot-mouth disease - Coxsackie A-16 virus (Picornavirus: |
| | Enterovirus) |
| | Hansen's disease - leprosy- Mycobacterium leprae (Acid-fast positive) |
| | Hantaan-Korean hemorrhagic fever - Hantavirus (Bunyavirus) |
| | Hantavirus Pulmonary Syndrome (HPS) - Hantavirus (Bunyavirus) |
| | Hard chancre - syphilis - Treponema pallidum subsp. pallidum |
| | Hard measles- Rubeola- measles- 14-day measles - Morbilli- Rubeola |
| | virus |
| | Haverhill fever - Rat bite fever - Streptobacillus moniliformis (G−; rod) |
| | Heartland fever - Heartland virus (phlebovirus)- transmitted by lone star |
| | tick- only two reported cases in Northwest Missouri |
| | Helicobacterosis - duodenal ulcers - Helicobacter pylori (G− curved rod) |
| | Hemolytic Uremic Syndrome- Hamburger disease- Escherichia coli |
| | O157 H7 strain. |
| | Hepatitis A - hepatitis A virus (Picornavirus: Enterovirus) |
| | Hepatitis B - hepatitis B virus (Hepadnavirus) |
| | Hepatitis C - hepatitis C virus (Flavivirus) |
| | Hepatitis D - hepatitis D virus (Deltavirus) |
| | Hepatitis E - hepatitis E virus (Calicivirus) |
| | Herpangina (*) - Coxsackie A (Picornavirus: Enterovirus), Enterovirus 7 |
| | (Picornavirus: Enterovirus) |
| | Herpes, genital - HSV-2 (Herpesvirus) |
| | Herpes labialis - HSV-1 (Herpesvirus) |
| | Herpes, neonatal - HSV-2 (Herpesvirus) |
| | Hidradenitis - Staphylococcus aureus (G+ coccus) |
| | HIV - human immunodeficiency virus (Retrovirus) |
| | Histoplasmosis - Histoplasma capsulatum (dimorphic fungus) |
| | His-Werner disease, Quintana fever, 5-day fever, Trench fever, Shinbone |
| | fever, Wolhynia fever- Bartonella quintana (G− rod) |
| | Hookworm infections - Ancylostoma duodenale, Necator americanus |
| | (intestinal nematode) |
| | Hordeola- Stye- Staphylococcus aureus |
| | HTLV- associated myelopathy (HAM) - Human T-cell Leukemia viruses |
| | I or II (retrovirus) |
| | Human Pulmonary Syndrome (HPS) - Four Corners Disease - Sin |
| | Nombre Virus (Hantaan virus group; Bunyavirus) |
| | Human monocytic ehrlichiosis - Ehrlichia chaffeensis. (G− intracellular |
| | bacteria) transmitted by ticks |
| | Human granulocytic ehrlichiosis - Ehrlichia equi. (G− intracellular |
| | bacteria) transmitted by ticks |
| | Hydatid cyst - Echinococcus granulosus, Echinococcus multilocularis, |
| | Echinococcus vogeli (larval cestode infection) |
| | Hydrophobia - Rabies - Rabies virus (Rhabdovirus) |
| | Impetigo- Streptococcus pyogenes, Staphylococcus aureus |
| | Inclusion conjunctivitis - Swimming Pool conjunctivitis- Pannus - |
| | Chlamydia trachomatis (G− intracellular) eye infection |
| | Infantile diarrhea- Escherichia coli (ETEC- enterotoxigenic E. coli) |
| | Infectious Mononucleosis - Epstein-Barr virus (Herpesvirus; HHV-4) |
| | Infectious myocarditis (*) - Coxsackie B1-B5 (Picornavirus: |
| | Enterovirus) |
| | Infectious pericarditis (*)- Coxsackie B1-B5 (Picornavirus: Enterovirus) |
| | Influenza- Flu - Influenza viruses A, B, and C (Orthomyxovirus) |
| | Israeli spotted fever - unnamed Rickettsia (G− intracellular; tick-borne) |
| | Isosporiasis- Isospora belli (protozoan) |
| (J) | Japanese B encephalitis virus - JEE virus (Flavivirus) |
| | Jock itch - Tinea cruris - Microsporum, Trichophyton, and |
| | Epidermophyton (fungi) |
| | Jorge Lobo disease - lobomycosis, Lobo's mycosis, Keloidal |
| | blastomycosis - Paracoccidioides loboi (Fungus) |
| | Jungle yellow fever, Yellow fever, Sylvatic yellow fever, Urban yellow |
| | fever, Vomito negro, Yellow Jack, Yellow fever virus- Flaviviridae, |
| | Flavivirus |
| | Junin Argentinian hemorrhagic fever - Juninvirus (Arenavirus) |
| (K) | Kala Azar - Visceral Leishmaniasis - Leishmania leishmania donovani, |
| | L. leishmania infantum, L. leishmania chagasi (protozoan parasite) |
| | sandfly |
| | Keratoconjunctivitis (*) - Viral conjunctivitis- Adenovirus (Adenovirus), |
| | HSV-1 (Herpesvirus) |
| | Kaposi's sarcoma - Human Herpes Virus 8 (Herpesvirus) or Kaposi's |
| | Sarcoma-associated Herpes Virus (KSHV) |
| | Kuru - prion (a protein) |
| | Kyasanur forest disease - KFD virus (flavivirus) tick-borne |
| (L) | LaCrosse encephalitis - LaCross virus (Bunyavirus) |
| | Lassa hemorrhagic fever - Lassavirus (Arenavirus) |
| | Legionnaire's pneumonia - Legionella pneumophila (G− rod: facultative- |
| | straight: respiratory pathogens) |
| | Lemming fever- tularemia, rabbit fever, deer fly fever, O'Hara disease, |
| | Francis disease, Francisella tularensis (G− rods: facultative-straight: |
| | zoonoses) |
| | Leprosy (Hansen's disease) - Mycobacterium leprae (Acid-fast positive) |
| | Leptospirosis -Weil's disease- canicola fever- canefield fever- |
| | nanukayami fever- 7-day fever- Leptospira interrogans (spiral shaped |
| | bacteria) |
| | Lemierre's Syndrome- Fusobacterium necrophorum (G− rod; anaerobe) |
| | Listerosis - Listeria monocytogenes (G+ rod) |
| | Liver fluke infection - Clonorchis sinensis, Opisthorchis viverrini, O. |
| | felineus, Fasciola hepatica (liver flukes) |
| | Lockjaw - Tetanus - Clostridium tetani (G+ rod; anaerobe) |
| | Loiasis - Eyeworm - Loa loa (parasitic worm) |
| | Louping Ill - Flavivirus (arbovirus) ticks |
| | Ludwig's angina- usually a polymicrobial infection (cellulitis of the floor |
| | of the mouth with spread to the submental, sublingual and submandibular |
| | spaces). Bacteria from mouth. |
| | Lung fluke infection - Paragonimus westermani |
| | Lyme disease - Borrelia burgdorferi (Spirochetes) |
| | Lyme-like illness- Masters disease- Southern tick associated rash illness |
| | (STARI)- Borrelia lonestari (possible etiology) |
| | Lymphogranuloma venereum (LGV) - Chlamydia trachomatis |
| | (intracellular G− bacteria; the L serotypes) |
| (M) | Machupo Bolivian hemorrhagic fever - Machupovirus (Arenavirus) |
| | Madura foot- Eumycotic mycetoma- Pseudallescheria boydii, |
| | Madurella grisea, Madurella mycetomatis (fungi) |
| | Malaria - Plasmodium sp. (protozoan parasite) |
| | Mal del pinto - Pinta - Treponema pallidum var. carateum |
| | Malignant pustule- Black Bane- Anthrax- Wool sorter's disease- Tanner's |
| | disease- Bacillus anthracis (G+ rod: sporulating: aerobic) |
| | Malta fever - Brucellosis- Brucella sp. (G− rods: facultative-straight: |
| | zoonoses) |
| | Marburg hemorrhagic fever - Marburg virus (Filovirus) |
| | Masters disease- Southern tick associated rash illness (STARI)- Lyme- |
| | like illness- Borrelia lonestari (possible etiology) |
| | Measles - Morbilli- Hard measles- Rubeola- measles- 14-day measles- |
| | rubeola virus (Paramyxovirus) |
| | Mediterannean spotted fever- Rickettsia coronii, (G−; intracellular |
| | bacteria) |
| | Melioidosis - Whitmore's disease- Burkholderia pseudomallei (used to |
| | be called Pseudomonas pseudomallei; G− rod: aerobic) |
| | MERS (Middle East Respiratory Syndrome)- Coronavirus called |
| | MERS-CoV |
| | Meningitis, aseptic (*) - Coxsackie A and B (Picornavirus: Enterovirus), |
| | Echovirus (Picornavirus: Enterovirus), lymphocytic choriomeningitis |
| | virus (Arenavirus), HSV-2 (Herpesvirus), Mycobacterium tuberculosis |
| | (Acid-fast) |
| | Meningitis, bacterial (*) - Neisseria meningitidis (G− cocci), |
| | Haemophilus influenzae (G− rod: facultative-straight: respiratory |
| | pathogens), Listeria monocytogenes (G+ rod: non-sporulating: non- |
| | filamentous), Streptococcus pneumoniae (G+ cocci), Group B |
| | streptococcus (G+ cocci) |
| | Milker's nodule - Parapoxvirus |
| | Middle East Respiratory Syndrome (MERS)- Coronavirus called MERS- |
| | CoV |
| | Molluscum contagiosum - Molluscipoxvirus (Poxvirus) |
| | Moniliasis- candidiasis- infection of the mucous membranes caused by |
| | the yeast Candida albicans. |
| | Monkeypox- Monkeypox virus- Poxviridae- Chordopoxvirus |
| | Mononucleosis - Epstein-Barr virus (Herpesvirus; HHV-4) |
| | Mononucleosis-like syndrome (*) - Cytomegalovirus (CMV; |
| | Herpesvirus; HHV-5) |
| | Montezuma's Revenge- Traveler's diarrhea - Any number of bacteria |
| | (Escherichia coli, Salmonella, Shigella, Yersinia, Vibrio, etc.), viruses |
| | (Rotaviruses, Norwalk-like agents), or parasites (Giardia, Entamoeba, |
| | Cryptosporidium)that cause diarrhea. |
| | Morbilli- Hard measles- Rubeola- measles- 14-day measles - Rubeola |
| | virus |
| | Mucormycosis- Zygomycosis- Rhizopus arrhizus (fungus) |
| | Multiple Organ Dysfunction Syndrome or MODS (*)- if infectious see |
| | Septic Shock for common causes. |
| | Mumps - mumps virus (Paramyxovirus) |
| | Murine typhus - Rickettsia typhi (G− intracellular; rodents and fleas) |
| | Murray Valley encephalitis - Flavivirus (arbovirus) mosquito |
| | Mycoburuli ulcers- Buruli ulcers- Mycobacterium ulcerans |
| | Mycotic vulvovaginitis- Candida albicans (yeast) |
| | Myositis- Streptococcus pyogenes, Staphylococcus aureus |
| (N) | Nanukayami fever- leptospirosis -Weil's disease- canicola fever- |
| | canefield fever-7-day fever- Leptospira interrogans (spiral shaped |
| | bacteria) |
| | Negishi - Flavivirus (arbovirus) vector unknown |
| | Necrotizing fasciitis- Type 1 = Streptococcus pyogenes: Type 2 = |
| | Staphylococcus aureus |
| | New world spotted fever, Rocky Mountain spotted fever, Sao Paulo |
| | fever - Rickettsia rickettsii (Obligate intracellular) |
| | Nocardiosis - Nocardia (G+: non-sporulating: filamentous) |
| | Nongonococcal urethritis(*) - Chlamydia trachomatis (G−; intracellular |
| | bacteria), Mycoplasma genitalium (bacterium without a cell wall), |
| | Ureaplasma urealyticum (bacterium without a cell wall), Gardnerella |
| | vaginalis (G variable rod), Trichomonas vaginalis (protozoan parasite), |
| | and Herpes Simplex virus (herpes virus) |
| | North American blastomycosis- Gilchrist's disease- Chicago disease- |
| | Blastomycosis- Blastomyces dermatitidis (dimorphic fungus) |
| | North Asian tick typhus - Rickettsia sibirica (G− intracellular; tick-borne) |
| | Norwegian itch - Scabies - Sarcoptes scabiei (parasitic mite) |
| (O) | O'Hara disease, deer fly fever, tularemia, lemming fever, rabbit fever, |
| | Francis disease, Francisella tularensis (G− rods: facultative-straight: |
| | zoonoses) |
| | Omsk hemorrhagic fever - OHF virus (Flavivirus; tick borne) |
| | Onchoceriasis - River Blindness - Onchocerca volvulus (parasitic worm) |
| | Onychomycosis- Tinea unguium - Ringworm of the nails- Trichophyton |
| | sp., and Epidermophyton floccosum (fungi) |
| | Opisthorchiasis - Liver fluke infection - Opisthorchis viverrini, O. |
| | felineus (liver flukes) |
| | Opthalmia neonatorium - Gonorrhea - Neisseria gonorrhoeae (G− cocci) |
| | Ornithosis - Parrot fever - Psittacosis - Chlamydia psittaci (G− |
| | intracellular) |
| | Oral hairy leukoplakia - Epstein Barr Virus (Human Herpes virus 4) |
| | Oriental Spotted Fever - Rickettsia japonica (G− intracellular; tick-borne) |
| | Oriental Sore - Leishmania leishmania major and L. leishmania tropica |
| | (protozoan parasite) sandfly |
| | Orf - Orfvirus (Poxvirus) |
| | Oroya fever - Carrion disease - Bartonellosis - Bartonella bacilliformis |
| | (weak G− polymorphic) sandfly bites at elevations of 600 to 2800 meter |
| | in Peru, Ecuador and Colombia. |
| | Otitis media- Streptococcus pneumoniae, Haemophilus influenzae, |
| | Moraxella catarrhalis, various viruses. |
| | Otitis externa (*) - Pseudomonas aeruginosa (G− rod: aerobic) |
| (P) | Parotitis - Mumps - Mumps virus (paramyxovirus) |
| | Paronychia - Candida albicans (yeast), Herpes Simplex virus (herpes |
| | virus) |
| | Parrot fever - Ornithosis- Psittacosis - Chlamydia psittaci (G− |
| | intracellular) |
| | Pannus - Chlamydia trachomatis (G− intracellular) eye infection |
| | Paragonimiasis - Lung fluke infection - Paragonimus westermani |
| | Paracoccidioidomycosis - Paracoccidioides brasiliensis (dimorphic |
| | fungi) |
| | PCP pneumonia- Pneumonia caused by Pneumocystis carinii |
| | Pediculosis - lice |
| | Peliosis hepatica - Bartonella henselae (pleomorphic G−) |
| | Pelvic Inflammatory Disease (PID) - two most common = Neiserria |
| | gonorrhoeae (G− coccus), Chlamydia trachomatis, then Anaerobic |
| | bacteria (ex. Bacteroides), Facultative Gram negative rods (ex. E. coli), |
| | Mycoplasma hominis, Actinomyces israelii (IUD recipients: G+ rod) |
| | Pertussis - Whooping cough- Bordetella pertussis (G− rods: facultative- |
| | straight: respiratory pathogens) |
| | Pharyngoconjunctival fever (*) - Adenovirus 1-3 and 5 (Adenovirus) |
| | Phaeohyphomycosis(*) - over 75 different species of fungi, most |
| | common = Phaeoaellomyces werneckii and P. hortae |
| | Piedra- Black Piedra = Piedraia hortai, White Piedra = Trichosporon |
| | beigelii |
| | Pigbel- beta-toxin of Clostridium perfringens type C |
| | “Pink eye” conjunctivitis (*) - Haemophilus aegyptius (G− rod: |
| | facultative-straight: respiratory pathogens) and/or Moraxella lacunata |
| | (G− diplococcus) |
| | Pinta - Treponema pallidum var. carateum |
| | Pinworm infection - Enterobiasis - Enterobius vermicularis (intestinal |
| | nematode) |
| | Pitted Keratolysis - Micrococcus sedentarius (G+ coccus) |
| | Pityriasis versicolor- Tinea versicolor- Malassezia furfur (fungus) |
| | Plague - Yersinia pestis (G− rod: facultative-straight: zoonoses) |
| | Pleurodynia - Coxsackie B (Picornavirus: Enterovirus) |
| | Pneumonia, viral (*) - respiratory syncytial virus (Paramyxovirus), CMV |
| | (Herpesvirus) |
| | Pneumocystosis - Pneumocystis carinii (protozoan parasite) |
| | Polio or Poliomyelitis - Polioviruses types I, II, and III (picornavirus) |
| | Polycystic hydatid - Echinococcus vogeli (larval cestode infection) |
| | Pontiac fever - Legionella pneumophila (G− rod: facultative-straight: |
| | respiratory pathogens) |
| | Pork tapeworm - Taenia solium |
| | Posada-Wernicke disease- Desert rheumatism- Coccidioidomycosis- San |
| | Joaquin Valley fever- Coccidioides immitis (dimorphic fungus) |
| | Postanginal septicemia- Lemierre's Syndrome- Fusobacterium |
| | necrophorum (G− rod; anaerobe) |
| | Powassan - Flavivirus (arbovirus) ticks |
| | Progressive multifocal leukencephalopathy - JC virus (Papovavirus) |
| | Progressive Rubella Panencephalitis - Rubella virus (togavirus) |
| | Prostatitis, bacterial(*) - most common = Escherichia coli, Klebsiella sp., |
| | Proteus sp., Pseudomonas sp., Enterobacter sp., Serratia sp., (G− rods), |
| | Enterococcus feacalis (G+ coccus) |
| | Pseudomembranous colitis - Clostridium difficile (G+ rod: sporulating: |
| | anaerobic) |
| | Psittacosis - Chlamydia psittaci (G− intracellular) |
| | Puerperal fever- Streptococcus pyogenes |
| | Pyelonephritis(*) - similar to cystitis |
| | Pylephlebitis - Bateroides fragilis (G− anaerobic rod), |
| | Peptostreptococcus spp (G+ anaerobic cocci), Clostridium spp. (G+ |
| | anaerobic rods), and several of the Enterobacteriaceae (G− rods; ferment |
| | glucose) |
| (Q) | Q fever - Coxiella burnetti (Obligate intracellular: Rickettsia) |
| | Australian tick typhus- Australian Spotted Fever- Queensland Tick |
| | Typhus- Rickettsia australis, (G−; intracellular bacteria) |
| | Quinsy- Peritonsillar abscess- a complication of untreated Strep. throat |
| | (Streptococcus pyogenes) |
| | Quintana fever, 5-day fever, Trench fever, Shinbone fever, Wolhynia |
| | fever, His-Werner disease- Bartonella quintana (G− rod) |
| (R) | Rabies - rabies virus (Rhabdovirus) |
| | Rabbit fever- deer fly fever, tularemia, lemming fever, O'Hara disease, |
| | Francis disease, Francisella tularensis (G− rods: facultative-straight: |
| | zoonoses) |
| | Racoon roundworm infection- Baylisascaris infection - Baylisascaris |
| | procyonis |
| | Rat bite fever - Streptobacillus moniliformis (G−; rod) |
| | Rat tapeworm - Hymenolepis diminuta |
| | Reiter Syndrome (*)- resulting from a nongonococcal sexually |
| | transmitted disease due usually to Chlamydia trachomatis or from an |
| | infectious diarrhea (Shigella, Salmonella, Yersinia). Persons with an |
| | HLA-B27 major histocompatibility complex are more likely to get this |
| | disease. |
| | Relapsing fever- Borrelia recurrentis |
| | Relapsing fever-like disease- Borrelia miyamotoi |
| | Rheumatic fever - Streptococcus pyogenes (nonsuppurative complication |
| | of Strep throat) |
| | Rhodotorulosis - Rhodotorula spp. (fungus) |
| | Rickettsialpox - Rickettsia akari (G−; intracellular) from mite bites |
| | Rift Valley Fever- Rift valley fever virus- Bunyavirus- Phlebovirus |
| | Ringworm - Microsporum, Trichophyton, and Epidermophyton (fungi) |
| | River Blindness - Onchoceriasis - Onchocerca volvulus (parasitic worm) |
| | Ritter's Disease- Filatow-Dukes' Disease, Scalded Skin Syndrome- |
| | Staphylococcus aureus- (exfoliative toxin producing strains) |
| | Rocky Mountain spotted fever, New world spotted fever, Sao Paulo |
| | fever - Rickettsia rickettsii (Obligate intracellular) |
| | Rose Handler's disease - Sporotrichosis - Sporothrix schenckii |
| | (dimorphic fungi) |
| | Rose rash of infants- Sixth disease - Zahorsky's disease - Roseola |
| | infantum - Exanthem subitum - “Sudden Rash”- 3-day fever- Human |
| | Herpes virus 6 (HHV-6) |
| | Roseola - Roseola infantum - Sixth disease - Zahorsky's disease - |
| | Exanthem subitum - Human Herpes virus 6 (HHV-6) |
| | Roundworm infections - Ascariasis - Ascaris lumbricoides (intestinal |
| | nematode) |
| | Rotavirus infections - Rotavirus (reovirus) |
| | Rubella - German measles- 3-day measles- rubella virus (Togavirus) |
| | Rubeola-measles- 14-day measles- Hard measles- Morbilli- Rubeola |
| | virus |
| | Russian spring-summer encephalitis- Far Eastern tick-borne encephalitis- |
| | Spring-summer encephalitis- Taiga encephalitis- Russian spring-summer |
| | encephalitis virus- Flaviviridae |
| (S) | Salmonellosis - Salmonella spp. (G− rod) |
| | San Joaquin Valley fever- Posada-Wernicke disease- Desert rheumatism- |
| | Coccidioidomycosis- Coccidioides immitis (dimorphic fungus). |
| | Sao Paulo Encephalitis - Flavivirus (arbovirus) |
| | Sao Paulo fever, New world spotted fever, Rocky Mountain spotted |
| | fever- Rickettsia rickettsii (Obligate intracellular) |
| | SARS- Severe Acute Respiratory Syndrome- SARS-associated |
| | coronavirus or SARS-CoV |
| | Scabies - Norwegian itch - Sarcoptes scabiei (parasitic mite) |
| | Scarlet fever - Scarlatina- Streptococcus group A (Streptococcus |
| | pyogenes) |
| | Scarlatina- Scarlet fever - Streptococcus group A (Streptococcus |
| | pyogenes) |
| | Scalded Skin Syndrome- Ritter's Disease- Filatow-Dukes' Disease- |
| | Staphylococcus aureus- (exfoliative toxin producing strains) |
| | Schistosomiasis - Schistosoma mansoni, S. japonicum, and S. |
| | haematobium (protozoan parasites; blood flukes) |
| | Scrub typhus - Rickettsia tsutsugamushi (G− intracellular; chigger bite) |
| | Sennetsu fever - Ehrlichiosis - Ehrlichia sp. (G− intracellular bacteria) |
| | transmitted by ticks |
| | Sepsis- See Septic Shock below. |
| | Septic Shock(*) - Most are due to bacterial infections. 50% due to Gram |
| | negative bacteria; 50% due to Gram positive bacteria. It depends on the |
| | location of the site of the initial infection. Most common sites of |
| | infection leading to sepsis are lungs, abdomen, and urinary tract (ex. |
| | urinary tract think Escherichia coli; community acquired pneumonia |
| | think Streptococcus pneumoniae). |
| | 7-day fever- Weil's disease - leptospirosis - canicola fever- canefield |
| | fever- nanukayami fever- Leptospira interrogans (spiral shaped bacteria) |
| | Severe Acute Respiratory Syndrome- SARS-coronavirus or SARS-CoV |
| | Shigellosis - Shigella sp. (G− rod) |
| | Shingles (zoster) - varicella zoster virus (Herpesvirus) |
| | Shipping fever - Pasteurella multocida (G− rods: facultative-straight: |
| | zoonoses) |
| | Siberian tick typhus- Rickettsia sibirica, (G−; intracellular bacteria) |
| | Sinusitis(*) - most common causes overall are respiratory viruses; most |
| | common bacterial causes = Streptococcus pneumoniae (G+ coccus) and |
| | Haemophilus influenzae (G− pleomorphic rod) (renamed and now called |
| | acute rhinosinusitis or acute bacterial rhinosinusitis) |
| | Sixth disease - Zahorsky's disease - Roseola infantum - Exanthem |
| | subitum - “Sudden Rash”- 3-day fever- Rose rash of infants- Human |
| | Herpes virus 6 (HHV-6) and HHV-7 (occasionally) |
| | “Slapped cheek” disease (erythema infectiosum; Fifth disease) - |
| | Parvovirus B19 (Parvovirus) |
| | Sleeping sickness- viral encephalitis - Mumps virus, Human Herpes |
| | virus 1, any of 350 different Arboviruses, Poxvirus, Enteroviruses (polio, |
| | Coxsackie, ECHO), Adenoviruses, Human Immunodeficiency Virus |
| | (retrovirus) |
| | Smallpox - variola virus (Poxvirus) - no naturally acquired cases since |
| | October 1977; Somalia |
| | Snail Fever- Schistosoma (protozoan parasite) |
| | Soft chancre - Chancroid - Haemophilus ducreyi (G− rod: facultative- |
| | straight: respiratory pathogens) |
| | Southern tick associated rash illness (STARI)- Lyme-like illness- |
| | Masters disease- Borrelia lonestari (possible etiology) |
| | Sparganosis - Spirometra sp. (cestode larvae infection) |
| | Spelunker's disease- Cave disease- Darling's Disease- Histoplasmosis- |
| | Histoplasma capsulatum (dimorphic fungus) |
| | Spotted fever- same as meningitis (bacterial) |
| | Sporadic typhus- Rickettsia prowazekii, (G−, intracellular bacterium; |
| | spread by fleas) |
| | Sporotrichosis - Sporothrix schenckii (dimorphic fungi) |
| | Spring-summer encephalitis- Far Eastern tick-borne encephalitis- |
| | Russian spring-summer encephalitis- Taiga encephalitis- Russian spring- |
| | summer encephalitis virus- Flaviviridae |
| | St. Louis encephalitis - SLE virus (Flavivirus) |
| | Strep. throat- Streptococcus pyogenes (G+ coccus). |
| | Stye- Hordeola- Staphylococcus aureus |
| | Strongyloiciasis - Threadworm - Strongyloides stercoralis (intestinal |
| | nematode) |
| | Subacute Sclerosing Panencephalitis (SSPE) - Measles virus |
| | Sudden Acute Respiratory Syndrome- SARS-CoV- Coronavirus |
| | “Sudden Rash”- 3-day fever- Exanthem subitum - Roseola infantum - |
| | Sixth disease - Zahorsky's disease- Rose rash of infants- Human Herpes |
| | virus 6 (HHV-6) |
| | Swimmer's ear- Otitis externa- Pseudomonas aeruginosa (common in |
| | diabetic patients) |
| | Swimmer's Itch - Schistosoma avium (bird schistosomes) (protozoan |
| | parasite) |
| | Swimming Pool conjunctivitis- Inclusion conjunctivitis - Pannus - |
| | Chlamydia trachomatis (G− intracellular) eye infection |
| | Swine flu- Influenza virus H1N1 |
| | Syphilis - Treponema pallidum subsp. pallidum (Spirochetes; bacteria) |
| | Systemic Inflammatory Response Syndrome or SIRS (*)- if infectious |
| | see Septic Shock for common causes. |
| | Sylvatic yellow fever, Yellow Jack, Jungle yellow fever, Yellow fever, |
| | Urban yellow fever, Vomito negro, Yellow fever virus- Flaviviridae, |
| | Flavivirus |
| (T) | Tabes dorsalis - tertiary syphilis - Treponema pallidum subsp. pallidum |
| | (Spirochetes) |
| | Taeniasis - see Tapeworm infections with Taenia species. |
| | Taiga encephalitis- Russian spring-summer encephalitis- Far Eastern |
| | tick-borne encephalitis- Spring-summer encephalitis- Russian spring- |
| | summer encephalitis virus- Flaviviridae |
| | Tanner's disease - Wool sorters' disease- Malignant pustule- Black Bane- |
| | Bacillus anthracis (G+ rod: sporulating: aerobic) |
| | Tapeworm infections - Taenia solium (pork tapeworm), Taenia saginata |
| | (beef tapeworm), Diphyllobothrium latum (fish tapeworm), Hymenolepis |
| | nana (dwarf tapeworm), Hymenolepis diminuta (rat tapeworm), |
| | Diphylidium caninum (dog tapeworm) (intestinal cestodes) |
| | TB- Tuberculosis - Mycobacterium tuberculosis (Acid-fast bacterium) |
| | Temporal lobe encephalitis (*) - HSV-1 (Herpesvirus) |
| | Tetanus - Clostridium tetani (G+ rod: sporulating: anaerobic) |
| | Threadworm infections - Strongyloiciasis - Strongyloides stercoralis |
| | (intestinal nematode) |
| | 3-day fever- Exanthem subitum - Roseola infantum - Sixth disease - |
| | Zahorsky's disease- “Sudden Rash”, Rose rash of infants- Human Herpes |
| | virus 6 (HHV-6) |
| | 3-day measles- German measles- Rubella- Rubella virus |
| | Thrush - Candida albicans (yeast) |
| | Tick-borne encephalitis- Biphasic meningoencephalitis, Central |
| | European tick-borne encephalitis, Czechoslovak tick-borne encephalitis, |
| | Diphasic milk fever, Viral meningoencephalitis, Tick-borne encephalitis |
| | virus- Flaviviridae |
| | Tick typhus- Fievre boutonneuse- Rickettsia conori |
| | Tinea barbae - Trichophyton verrucosum, T. mentagrophytes, T. rubrum, |
| | T. megninii (fungi) |
| | Tinea capitis - Ringworm of the head- Microsporum sp., Trichophyton |
| | sp. (fungi) |
| | Tinea corporis - Ringworm of the body- Microsporum, Trichophyton, |
| | and Epidermophyton floccosum (fungi) |
| | Tinea manuum - Ringworm of the hand- Trichophyton sp., and |
| | Epidermophyton floccosum (fungi) |
| | Tinea cruris - Ringworm of the groin- Candida albicans (yeast), |
| | Trichophyton sp., and Epidermophyton floccosum (fungi) |
| | Tinea nigra- Exophiala werneckii |
| | Tinea pedis - Ringworm of the feet- Trichophyton sp., and |
| | Epidermophyton floccosum(fungi) |
| | Tinea unguium - Onychomycosis- Ringworm of the nails- Trichophyton |
| | sp., and Epidermophyton floccosum (fungi) |
| | Tinea versicolor- Pityriasis versicolor- Malassezia furfur (fungus) |
| | Torulopsosis - Torulopsis glabrata and T. candida (fungus) |
| | Torulosis- Busse-Buschke disease- Cryptococcosis- European |
| | blastomycosis- Cryptococcus neoformans (encapsulated yeast) |
| | Toxic Shock Syndrome - Staphylcoccus aureus (G+ cocci; producing |
| | TSST) and Streptococcus pyogenes (G+ cocci) |
| | Toxoplasmosis - Toxoplasma gondii (protozoan parasite) |
| | Traveler's diarrhea - Any number of bacteria (Escherichia coli (most |
| | common), Salmonella, Shigella, Yersinia, Vibrio, etc.), viruses |
| | (Rotaviruses, Norwalk-like agents), or parasites (Giardia, Entamoeba, |
| | Cryptosporidium) that cause diarrhea. |
| | Trench fever, 5-day fever, Shinbone fever, Wolhynia fever, Quintana |
| | fever, His-Werner disease- Bartonella quintana (G− rod) |
| | Trench mouth or Vincent's disease- Various anaerobic bacteria in the |
| | mouth |
| | Trichinellosis- Trichinella spiralis (nematode parasite) |
| | Trichomoniasis - Vaginitis - Trichomonas vaginalis (protozoan parasite) |
| | Trichomycosis axillaris - Corynebacterium tenuis (G+ rod) |
| | Trichuriasis - Whipworm infection - Trichuris trichiura (intestinal |
| | nematode) |
| | Tropical Spastic Paraparesis (TSP) - Human T-cell Leukemia viruses I or |
| | II (retrovirus) |
| | Trypanosomiasis - African = Trypanosoma brucei rhodesiense, |
| | Trypanosoma brucei gambiense (tsetse fly-borne), American = |
| | Trypanosoma cruzi(Triatomine bugs = kissing bug or assassin bugs) |
| | Tuberculosis - TB- Mycobacterium tuberculosis (Acid-fast bacterium) |
| | Tularemia- lemming fever, rabbit fever, deer fly fever, O'Hara disease, |
| | Francis disease, Francisella tularensis (G− rods: facultative-straight: |
| | zoonoses) |
| | Typhoid fever - Salmonella typhi (G− rod: facultative-straight: enteric |
| | pathogens) |
| | Typhus fever - Rickettsia prowazekii (G− intracellular; louse-borne), |
| | Rickettsia typhi (G− intracellular; flea-borne) |
| (U) | Ulcus molle - Soft chancre - Chancroid - Haemophilus ducreyi (G− rod: |
| | facultative-straight: respiratory pathogens) |
| | Undulant fever - Brucella sp. (G− coccobacillus: zoonoses) |
| | Urban yellow fever, Sylvatic yellow fever, Yellow Jack, Jungle yellow |
| | fever, Yellow fever, Vomito negro, Yellow fever virus- Flaviviridae, |
| | Flavivirus |
| | Urethritis - Herpes Simplex virus, Chlamydia trachomatis, Ureaplasma |
| | urealyticum, Neisseria gonorrhoeae |
| (V) | Vaginosis, bacterial - Peptostreptococccus sp., Bacteriodes sp., |
| | Gardnerella vaginalis, Mobiluncus sp., Mycoplasma sp. (clue cells) |
| | Vaginitis - Candida albicans (yeast; Mycotic vulvovaginitis), |
| | Trichomonas vaginalis (protozoan parasite; Trichomoniasis) |
| | Varicella -chickenpox - Varicella-Zoster virus (VZV or Human herpes 3 |
| | virus) |
| | Venezuelan Equine encephalitis - Togaviridae, Alphavirus |
| | Verruga peruana- Carrion's disease - Bartonellosis - Oroya fever - |
| | Bartonella bacilliformis (weak G− polymorphic) sandfly bites at |
| | elevations of 600 to 2800 meter in Peru, Ecuador and Colombia. |
| | Vincent's disease or Trench mouth- Various anaerobic bacteria in the |
| | mouth |
| | Viral conjunctivitis (*) - Keratoconjunctivitis - Adenovirus |
| | (Adenovirus), HSV-1 (Herpesvirus) |
| | Viral meningoencephalitis- Czechoslovak tick-borne encephalitis, |
| | Central European tick-borne encephalitis, Diphasic milk fever, Biphasic |
| | meningoencephalitis, Tick-borne encephalitis, Tick-borne encephalitis |
| | virus- Flaviviridae |
| | Viral rash- Duke's disease- Coxsackievirus or Echovirus |
| | Visceral Larval Migrans - Toxocara canis (parasitic nematode) |
| | Vomito negro, Urban yellow fever, Sylvatic yellow fever, Yellow Jack, |
| | Jungle yellow fever, Yellow fever, Yellow fever virus- Flaviviridae, |
| | Flavivirus |
| | Vulvovaginitis - Candida albicans (yeast), Trichomonas vaginalis |
| | (protozoan parasite), and the causes of bacterial vaginosis. |
| (W) | Warts - Papilloma viruses |
| | Waterhouse-Friderichsen syndrome - Neisseria meningitidis (G− cocci) |
| | Weil's disease - Leptospirosis - canicola fever- canefield fever- |
| | nanukayami fever- 7-day fever- Leptospira interrogans (spiral shaped |
| | bacteria) |
| | West Nile Fever- West Nile virus- Flavivirus Japanese Encephalitis |
| | Antigenic Complex |
| | Western equine encephalitis - WEE virus, Togaviridae, Alphavirus |
| | Whipple's disease - Tropheryma whippelii (G+ rod a actinomycete) |
| | Whipworm infection - Trichuriasis - Trichuris trichiura |
| | White Piedra- Trichosporon beigelii |
| | Whitmore's disease- Melioidosis - Burkholderia pseudomallei (used to |
| | be called Pseudomonas pseudomallei; G− rod: aerobic) |
| | Whitlow - paronchyia - Herpes simplex virus (herpesvirus) |
| | Whooping cough - Pertussis- Bordetella pertussis (G− small rod) |
| | Winter diarrhea - Rotavirus infections - Rotavirus (reovirus) |
| | Wolhynia fever, His-Werner disease, Quintana fever, 5-day fever, |
| | Trench fever, Shinbone fever- Bartonella quintana (G− rod) |
| | Wool sorters' disease - Anthrax- Tanner's disease- Malignant pustule- |
| | Black Bane- Bacillus anthracis (G+ rod: sporulating: aerobic) |
| (XYZ) | Yaws -Treponema pallidum var. pertenue (spirochete) |
| | Yellow fever, Jungle yellow fever, Sylvatic yellow fever, Urban yellow |
| | fever, Vomito negro, Yellow Jack, Yellow fever virus- Flaviviridae, |
| | Flavivirus |
| | Yellow Jack, Jungle yellow fever, Yellow fever, Sylvatic yellow fever, |
| | Urban yellow fever, Vomito negro, Yellow fever virus- Flaviviridae, |
| | Flavivirus |
| | Yersinosis - Yersinia enterocolitica |
| | Zahorsky's disease - Roseola infantum - Exanthem subitum - Sixth |
| | disease - Human Herpes virus 6 (HHV-6) |
| | Zika virus disease- Zika virus |
| | Zoster - shingles- Varicella-Zoster virus (VZV or Human herpes 3 virus) |
| | Zygomycosis- Mucormycosis- Rhizopus arrhizus (fungus) |
| Autoimmune | Examples of autoimmune diseases or disorders: acute disseminated |
| Diseases | encephalomyelitis (ADEM); Addison's disease; ankylosing spondylitis; |
| | antiphospholipid antibody syndrome (APS); aplastic anemia; autoimmune |
| | gastritis; autoimmune hepatitis; autoimmune thrombocytopenia; Behçet's |
| | disease; coeliac disease; dermatomyositis; diabetes mellitus type I; |
| | Goodpasture's syndrome; Graves' disease; Guillain-Barré syndrome (GBS); |
| | Hashimoto's disease; idiopathic thrombocytopenia purpura; inflammatory bowel |
| | disease (IBD) including Crohn's disease and ulcerative colitis; mixed connective |
| | tissue disease; multiple sclerosis (MS); myasthenia gravis; opsoclonus |
| | myoclonus syndrome (OMS); optic neuritis; Ord's thyroiditis; pemphigus; |
| | pernicious anaemia; polyarteritis nodosa; polymyositis; primary biliary cirrhosis; |
| | primary myoxedema; psoriasis; rheumatic fever; rheumatoid arthritis; Reiter's |
| | syndrome; scleroderma; Sjögren's syndrome; systemic lupus erythematosus; |
| | Takayasu's arteritis; temporal arteritis; vitiligo; warm autoimmune hemolytic |
| | anemia; or Wegener's granulomatosis. The MS may be any clinical variety or |
| | origin, and not limited to mammals. Non-limiting examples may include |
| | Experimental autoimmune encephalomyelitis (EAE), clinically isolated |
| | syndrome (CIS), Relapsing-remitting MS (RRMS), Secondary progressive MS |
| | (SPMS), or Primary progressive MS (PPMS). |
| | Examples of inflammatory diseases or disorders: asthma, allergy, allergic |
| | rhinitis, allergic airway inflammation, atopic dermatitis (AD), chronic obstructive |
| | pulmonary disease (COPD), inflammatory bowel disease (IBD), Irritable bowel |
| | syndrome (IBS), multiple sclerosis, arthritis, psoriasis, eosinophilic esophagitis, |
| | eosinophilic pneumonia, eosinophilic psoriasis, hypereosinophilic syndrome, |
| | graft-versus-host disease, uveitis, cardiovascular disease, pain, multiple sclerosis, |
| | lupus, vasculitis, chronic idiopathic urticaria and Eosinophilic Granulomatosis |
| | with Polyangiitis (Churg-Strauss Syndrome). |
| | The asthma may be allergic asthma, non-allergic asthma, severe refractory |
| | asthma, asthma exacerbations, viral-induced asthma or viral-induced asthma |
| | exacerbations, steroid resistant asthma, steroid sensitive asthma, eosinophilic |
| | asthma or non-eosinophilic asthma and other related disorders characterized by |
| | airway inflammation or airway hyperresponsiveness (AHR). The COPD may be |
| | a disease or disorder associated in part with, or caused by, cigarette smoke, air |
| | pollution, occupational chemicals, allergy or airway hyperresponsiveness. The |
| | allergy may be associated with foods, pollen, mold, dust mites, animals, or |
| | animal dander. The IBD may be ulcerative colitis (UC), Crohn's Disease, |
| | collagenous colitis, lymphocytic colitis, ischemic colitis, diversion colitis, |
| | Behcet's syndrome, infective colitis, indeterminate colitis, and other disorders |
| | characterized by inflammation of the mucosal layer of the large intestine or |
| | colon. The arthritis may be selected from the group consisting of osteoarthritis, |
| | rheumatoid arthritis and psoriatic arthritis. |
| Cancer | Examples of cancer include but are not limited to glioblastoma, melanoma, |
| | non-small cell lung cancer, head-and-neck cancer, prostate cancer, colon cancer, |
| | breast cancer, bladder cancer, ovarian cancer, cervical cancer, endometrial |
| | cancer, renal cancer and pancreatic cancer. |

In an embodiment, the one or more CREs of the present invention are specifically active in a particular metabolic state of a cell and thus can be used to detect cells that have undergone (or not) a metabolic switch. In an embodiment, the one or more CREs of the present invention are specifically active in a particular metabolic state of a cell that corresponds to an epithelial metabolic state and thus would be active in a cell that has not undergone Epithelial to Mesenchymal transition (EMT). In an embodiment, the one or more CREs of the present invention are specifically active in a particular metabolic state of a cell that corresponds to a mesenchymal metabolic state and thus would be active in a cell that has undergone EMT. See e.g., Brabletz et al., Nature Reviews Cancer. 18:128-134 (2018).

### Engineered Polynucleotides

In general, the CREs of the present invention can be operatively coupled to one or more polynucleotides. The one or more polynucleotides can encode one or more gene products. As used herein, “gene product” refers to any polynucleotide, polypeptide, and/or the like that is ultimately produced from transcribing a gene and optionally translating the transcript. As used herein, the term “encode” refers to principle that DNA can be transcribed into RNA, which can then be optionally translated into amino acid sequences that form peptides and polypeptides. Thus, a polynucleotide said to encode a e.g., gene product is a polynucleotide that can be transcribed by an in vitro or in vivo method into an RNA transcript, which in turn can be optionally translated into a polypeptide. It will be appreciated that RNA transcripts can have functionality without being translated into polypeptides. A protein-encoding polynucleotide is a polynucleotide that encodes an RNA product that is translated into the protein.

As used herein, “gene” refers to a hereditary unit corresponding to a sequence of DNA that occupies a specific location on a chromosome and that contains the genetic instruction for a characteristic(s) or trait(s) in an organism. The term gene can refer to translated and/or untranslated regions of a genome. “Gene” can refer to the specific sequence of DNA that is transcribed into an RNA transcript that can be translated into a polypeptide or be a catalytic RNA molecule, including but not limited to, tRNA, siRNA, piRNA, miRNA, long-non-coding RNA and shRNA.

As used interchangeably herein, “operatively linked”, “operably linked”, “operatively coupled”, and “operably coupled” in the context of polynucleotide molecules (e.g., DNA and RNA) vectors, and the like refers in certain contexts to the association (operational and/or physical associate) of one or more polynucleotides and one or more other regulatory and/or other polynucleotides useful for driving, inhibiting, and/or otherwise regulating expression, stabilization, replication, and the like of the transcribed or transcribable regions (coding and/or non-coding) of a nucleic acid that are positioned in the nucleic acid molecule in the appropriate positions relative to the region to be transcribed so as to effect the expression or other characteristic of the region to be transcribed. This same term can be applied to the arrangement of coding sequences, non-coding and/or transcription control elements (e.g., promoters, enhancers, and termination elements), and/or selectable markers in an expression vector. “Operatively linked” can also refer to an indirect attachment (i.e. not a direct fusion) of two or more polynucleotide sequences or polypeptides to each other via a linking molecule (also referred to herein as a linker).

Without being bound by theory, the CREs of the present invention can be used to drive and/or otherwise regulate expression of a polynucleotide to which one or more CREs of the present invention are operatively coupled in a cell type specific, cell state specific, tissue type specific, and/or environment specific manner. As is described in greater detail in the exemplary embodiments below, this can be leveraged for a variety of applications that are dependent upon the polynucleotide that is operatively coupled to the one or more CREs of the present invention. For example, where the polynucleotide component of the engineered polynucleotide of the present invention is therapeutic or encodes a therapeutic gene product, the CREs of the present invention can provide for cell type specific, cell state specific, tissue type specific, and/or environment specific expression and/or regulation of that therapeutic polynucleotide. In other contexts, such as where it is desirable to detect a particular cell type, cell state, tissue type, and/or environment, the polynucleotide component of the engineered polynucleotide of the present invention can encode a reporter transcript or polypeptide and the CREs of the present invention included in the engineered polynucleotide can drive or enhance expression of the reporter polynucleotide in the cell type, cell state, tissue type, and/or environment to be detected so as to produce a detectable signal in those cells.

As used in this context herein, “detectable signal” refers to any change or molecule generated that can be detected or otherwise measured or quantified in response to expression or regulation of the expression of the polynucleotide component of the engineered polynucleotides of the present invention. In an embodiment, the detectable signal is the polynucleotide component itself. For example, In an embodiment, the polynucleotide component can contain a barcode or can otherwise be sequenced so as to allow detection of cell type specific, cell state, tissue type, and/or environment specific expression or regulation of expression by the CRE(s) of the engineered polynucleotide. In an embodiment, the polynucleotide component encodes a reporter protein, such as an optically active or enzymatic protein that can produce an optically detectable signal. In an embodiment, the polynucleotide component encodes a protein that can modify a characteristic (e.g., genotype and/or phenotype) of a cell in which it is expressed. In this case, the signal can be the genotype or phenotypic change.

The engineered polynucleotides of the present invention can be included in vectors or vector systems, delivery vehicles, and/or the like, which are described in greater detail elsewhere herein. The engineered polynucleotides can be delivered, contained, and/or expressed in vitro (e.g., outside of a cell), in vivo (inside a cell and/or in an organism), ex vivo, or in situ.

It will be appreciated that any desired polynucleotide can be operatively coupled to one or more CREs of the present invention using any suitable polynucleotide de novo synthesis technique and/or recombinant engineering technique. To the extent that the polynucleotide component sequence is known or generated it can be operatively coupled to one or more CREs of the present invention and used as described and envisioned herein.

As used herein, “nucleic acid,” “nucleotide sequence,” and “polynucleotide” can be used interchangeably herein and can generally refer to a string of at least two base-sugar-phosphate combinations and refers to, among others, single- and double-stranded DNA, DNA that is a mixture of single- and double-stranded regions, single- and double-stranded RNA, and RNA that is a mixture of single- and double-stranded regions, hybrid molecules comprising DNA and RNA that may be single-stranded or, more typically, double-stranded or a mixture of single- and double-stranded regions. In addition, polynucleotide as used herein can refer to triple-stranded regions comprising RNA or DNA or both RNA and DNA. The strands in such regions can be from the same molecule or from different molecules. The regions may include all of one or more of the molecules, but more typically involve only a region of some of the molecules. One of the molecules of a triple-helical region often is an oligonucleotide. “Polynucleotide” and “nucleic acids” also encompass such chemically, enzymatically, or metabolically modified forms of polynucleotides, as well as the chemical forms of DNA and RNA characteristic of viruses and cells, including simple and complex cells, inter alia. For instance, the term polynucleotide as used herein can include DNAs or RNAs as described herein that contain one or more modified bases. Thus, DNAs or RNAs including unusual bases, such as inosine, or modified bases, such as tritylated bases, to name just two examples, are polynucleotides as the term is used herein. “Polynucleotide”, “nucleotide sequences” and “nucleic acids” also includes PNAs (peptide nucleic acids), phosphorothioates, phosphorodiamidate morpholino oligomers, and other variants of the phosphate backbone of native nucleic acids. Natural nucleic acids have a phosphate backbone, artificial nucleic acids can contain other types of backbones, but contain the same bases. Thus, DNAs or RNAs with backbones modified for stability or for other reasons are “nucleic acids” or “polynucleotides” as that term is intended herein. As used herein, “nucleic acid sequence” and “oligonucleotide” also encompass a nucleic acid and polynucleotide as defined elsewhere herein. In an embodiment, the polynucleotides are codon optimized. Codon optimization of polynucleotides is described elsewhere herein, see e.g., below with respect to “vector polynucleotides”. In an embodiment, the engineered polynucleotides are included in a vector or vector system. In an embodiment, the engineered polynucleotides are not included in a vector or vector system. In an embodiment, the engineered polynucleotides are contained in a delivery vehicle. Delivery vehicles are described in greater detail elsewhere herein.

As used herein, “expression” refers to the process by which polynucleotides are transcribed into RNA transcripts. In the context of mRNA and other translated RNA species, “expression” also refers to the process or processes by which the transcribed RNA is subsequently translated into peptides, polypeptides, or proteins. In some instances, “expression” can also be a reflection of the stability of a given RNA. For example, when one measures RNA, depending on the method of detection and/or quantification of the RNA as well as other techniques used in conjunction with RNA detection and/or quantification, it can be that increased/decreased RNA transcript levels are the result of increased/decreased transcription and/or increased/decreased stability and/or degradation of the RNA transcript. One of ordinary skill in the art will appreciate these techniques and the relation “expression” in these various contexts to the underlying biological mechanisms.

As used herein “increased expression” or “overexpression” are both used to refer to an increased expression of a gene or gene product thereof in a sample as compared to the expression of said gene or gene product in a suitable control. The term “increased expression” preferably refers to 10%, 20%, 30%, 40%, 50%, 60%, 70%, 80%, 90%, 100%, 110%, 120%, 130%, 140%, 150%, 160%, 170%, 180%, 190%, 200%, 210%, 220%, 230%, 240%, 250%, 260%, 270%, 280%, 290%, 300%, 310%, 320%, 330%, 340%, 350%, 360%, 370%, 380%, 390%, 400%, 410%, 420%, 430%, 440%, 450%, 460%, 470%, 480%, 490%, 500%, 510%, 520%, 530%, 540%, 550%, 560%, 570%, 580%, 590%, 600%, 610%, 620%, 630%, 640%, 650%, 660%, 670%, 680%, 690%, 700%, 710%, 720%, 730%, 740%, 750%, 760%, 770%, 780%, 790%, 800%, 810%, 820%, 830%, 840%, 850%, 860%, 870%, 880%, 890%, 900%, 910%, 920%, 930%, 940%, 950%, 960%, 970%, 980%, 990%, 1000%, 1010%, 1020%, 1030%, 1040%, 1050%, 1060%, 1070%, 1080%, 1090%, 1100%, 1110%, 1120%, 1130%, 1140%, 1150%, 1160%, 1170%, 1180%, 1190%, 1200%, 1210%, 1220%, 1230%, 1240%, 1250%, 1260%, 1270%, 1280%, 1290%, 1300%, 1310%, 1320%, 1330%, 1340%, 1350%, 1360%, 1370%, 1380%, 1390%, 1400%, 1410%, 1420%, 1430%, 1440%, 1450%, 1460%, 1470%, 1480%, 1490%, or/to 1500% or more increased expression relative to a suitable control.

As used herein “reduced expression” or “underexpression” refers to a reduced or decreased expression of a gene, such as a gene relating to an antigen processing pathway, or a gene product thereof in sample as compared to the expression of said gene or gene product in a suitable control. As used throughout this specification, “suitable control” is a control that will be instantly appreciated by one of ordinary skill in the art as one that is included such that it can be determined if the variable being evaluated an effect, such as a desired effect or hypothesized effect. One of ordinary skill in the art will also instantly appreciate based on inter alia, the context, the variable(s), the desired or hypothesized effect, what is a suitable or an appropriate control needed. In one embodiment, said control is a sample from a healthy individual or otherwise normal individual. By way of a non-limiting example, if said sample is a sample of a lung tumor and comprises lung tissue, said control is lung tissue of a healthy individual. The term “reduced expression” preferably refers to at least a 25% reduction, e.g., at least a 30%, 40%, 50%, 60%, 70%, 75%, 80%, 85%, 90%, 95%, 98% or 99% reduction, relative to such control.

### Example Engineered Therapeutic Polynucleotides

As previously mentioned, one or more CREs of the present invention can be operatively coupled to one or more polynucleotides, such as one or more therapeutic polynucleotides so as to spatially and/or temporally control expression of the one or more therapeutic polynucleotides. In an embodiment, an engineered therapeutic polynucleotide includes one or more CREs of the present invention and one or more therapeutic polynucleotides, wherein the one or more CREs is/are operatively coupled to the therapeutic polynucleotide. In an embodiment, one or more of the one or more CREs are identified CREs, engineered CREs, or both. In an embodiment, expression or other regulation of expression of the one or more therapeutic polynucleotides is specific to a cell type, cell state, tissue type, and or environment, which is mediated by the one or more CREs of the present invention. It will be appreciated that any therapeutic polynucleotide can be operably coupled to the one or more CREs of the present invention and that such a coupling will be within the skill and expertise of one of ordinary skill in the art in view of the description herein. In some embodiment, the therapeutic polynucleotide component of the engineered therapeutic polynucleotide comprises a replacement gene; encodes a therapeutic gene product; comprises or encodes a genetic modification system or component thereof; comprises or encodes an RNAi molecule; comprises or encodes an aptamer; or any combination thereof.

Exemplary diseases, such as genetic disease which can benefit from a gene or gene product replacement therapy, a therapeutic protein, genetic modification, RNAi therapy, an aptamer, or other therapeutic polynucleotide are described in greater detail elsewhere herein.

As used herein, “replacement gene” refers to a gene or portion thereof that is delivered so as to replace or supplement one or more defective copies of a gene. The replacement gene can produce normal gene products, and thus can relieve the deficiency generated by the one or more defective copies of a gene. In an embodiment, a replacement gene or portion thereof for any gene identified in Tables 5-6 herein can be included in the therapeutic polynucleotide. Other diseases where replacement gene therapies are described elsewhere herein.

In an embodiment, the therapeutic gene product can be an RNA and/or protein. In an embodiment, the RNA can be subsequently translated into protein or is itself a catalytic or functional RNA. In an embodiment, the protein is a replacement protein therapy. The replacement protein therapy can provide functional protein where there is a specific protein deficiency. In an embodiment, the therapeutic protein is an antibody or fragment thereof, affibodies, nanobodies, antigen binding fragments and/or the like. The therapeutic protein can be a protein hormone, neurotransmitter, receptor ligand, signaling protein, and/or the like that can, when expressed in an appropriate cell, provide a biological response.

The term “antibody” is used interchangeably with the term “immunoglobulin” herein, and includes intact antibodies, fragments of antibodies, e.g., Fab, F(ab′)2 fragments, and intact antibodies and fragments that have been mutated either in their constant and/or variable region (e.g., mutations to produce chimeric, partially humanized, or fully humanized antibodies, as well as to produce antibodies with a desired trait, e.g., enhanced binding and/or reduced Immunoglobulin Fc receptor (FcR) binding). “Antibody” includes monovalent and multivalent antibodies. The term “fragment” refers to a part or portion of an antibody or antibody chain comprising fewer amino acid residues than an intact or complete antibody or antibody chain. Fragments can be obtained via chemical or enzymatic treatment of an intact or complete antibody or antibody chain. Fragments can also be obtained by recombinant means. Exemplary fragments include Fab, Fab′, F(ab′)2, Fabc, Fd, dAb, VHH and scFv and/or Fv fragments.

As used herein, a preparation of antibody protein having less than about 50% of non-antibody protein (also referred to herein as a “contaminating protein”), or of chemical precursors, is considered to be “substantially free.” In an embodiment, a preparation of antibody protein having less than about 40%, 30%, 20%, 10% and more preferably 5% (by dry weight), of non-antibody protein, or of chemical precursors is considered to be substantially free. When the antibody protein or biologically active portion thereof is recombinantly produced, it is also preferably substantially free of culture medium, i.e., culture medium represents less than about 30%, preferably less than about 20%, more preferably less than about 10%, and most preferably less than about 5% of the volume or mass of the protein preparation.

As used herein, “nanobody” refers to a single-domain antibody fragment that is capable of specifically binding an antigen. Nanobodies can be engineered to have desired antigen-binding capabilities. Nanobodies can be based on heavy-chain or light-chain domains. See e.g. Arbabi Ghahroudi M, Desmyter A, Wyns L, Hamers R, Muyldermans S (September 1997). “Selection and identification of single domain antibody fragments from camel heavy-chain antibodies”. FEBS Letters. 414 (3): 521-6. doi: 10.1016/S0014-5793 (97) 01062-4; Ward E S, Güssow D, Griffiths A D, Jones P T, Winter G (October 1989). “Binding activities of a repertoire of single immunoglobulin variable domains secreted from Escherichia coli”. Nature. 341 (6242): 544-6 . . . doi: 10.1038/341544a0; Holt L J, Herring C, Jespers L S, Woolven B P, Tomlinson I M (November 2003). “Domain antibodies: proteins for therapy”. Trends in Biotechnology. 21 (11): 484-90. doi: 10.1016/j.tibtech.2003.08.007; Borrebaeck C A, Ohlin M (December 2002). “Antibody evolution beyond Nature”. Nature Biotechnology. 20 (12): 1189-90. doi: 10.1038/nbt1202-1189; Van de Broek B, Devoogdt N, D'Hollander A, Gijs H L, Jans K, Lagae L, et al. (June 2011). “Specific cell targeting with nanobody conjugated branched gold nanoparticles for photothermal therapy”. ACS Nano. 5 (6): 4319-28. doi: 10.1021/nn1023363.

As used herein, the term “antigen-binding fragment” refers to a polypeptide fragment of an immunoglobulin or antibody that binds antigen or competes with intact antibody (i.e., with the intact antibody from which they were derived) for antigen binding (i.e., specific binding). As such these antibodies or fragments thereof are included in the scope of the invention, provided that the antibody or fragment binds specifically to a target molecule.

It is intended that the term “antibody” encompass any Ig class or any Ig subclass (e.g., the IgG1, IgG2, IgG3, and IgG4 subclasses of IgG) obtained from any source (e.g., humans and non-human primates, and in rodents, lagomorphs, caprines, bovines, equines, ovines, etc.).

The term “Ig class” or “immunoglobulin class”, as used herein, refers to the five classes of immunoglobulin that have been identified in humans and higher mammals, IgG, IgM, IgA, IgD, and IgE. The term “Ig subclass” refers to the two subclasses of IgM (H and L), three subclasses of IgA (IgA1, IgA2, and secretory IgA), and four subclasses of IgG (IgG1, IgG2, IgG3, and IgG4) that have been identified in humans and higher mammals. The antibodies can exist in monomeric or polymeric form; for example, IgM antibodies exist in pentameric form, and IgA antibodies exist in monomeric, dimeric, or multimeric form.

The term “IgG subclass” refers to the four subclasses of immunoglobulin class IgG-IgG1, IgG2, IgG3, and IgG4 that have been identified in humans and higher mammals by the heavy chains of the immunoglobulins, V1-γ4, respectively. The term “single-chain immunoglobulin” or “single-chain antibody” (used interchangeably herein) refers to a protein having a two-polypeptide chain structure consisting of a heavy and a light chain, said chains being stabilized, for example, by interchain peptide linkers, which has the ability to specifically bind the antigen. The term “domain” refers to a globular region of a heavy or light chain polypeptide comprising peptide loops (e.g., comprising 3 to 4 peptide loops) stabilized, for example, by a B pleated sheet and/or intrachain disulfide bond. Domains are further referred to herein as “constant” or “variable”, based on the relative lack of sequence variation within the domains of various class members in the case of a “constant” domain, or the significant variation within the domains of various class members in the case of a “variable” domain. Antibody or polypeptide “domains” are often referred to interchangeably in the art as antibody or polypeptide “regions”. The “constant” domains of an antibody light chain are referred to interchangeably as “light chain constant regions”, “light chain constant domains”, “CL” regions or “CL” domains. The “constant” domains of an antibody heavy chain are referred to interchangeably as “heavy chain constant regions”, “heavy chain constant domains”, “CH” regions or “CH” domains). The “variable” domains of an antibody light chain are referred to interchangeably as “light chain variable regions”, “light chain variable domains”, “VL” regions or “VL” domains). The “variable” domains of an antibody heavy chain are referred to interchangeably as “heavy chain variable regions”, “heavy chain variable domains”, “VH” regions or “VH” domains). In an embodiment, the VH domain is a human VH domain.

The term “region” can also refer to a part or portion of an antibody chain or antibody chain domain (e.g., a part or portion of a heavy or light chain or a part or portion of a constant or variable domain, as defined herein), as well as more discrete parts or portions of said chains or domains. For example, light and heavy chains or light and heavy chain variable domains include “complementarity determining regions” or “CDRs” interspersed among “framework regions” or “FRs”, as defined herein.

The term “conformation” refers to the tertiary structure of a protein or polypeptide (e.g., an antibody, antibody chain, domain or region thereof). For example, the phrase “light (or heavy) chain conformation” refers to the tertiary structure of a light (or heavy) chain variable region, and the phrase “antibody conformation” or “antibody fragment conformation” refers to the tertiary structure of an antibody or fragment thereof.

As used herein, “affibody” refers to small (typically around 6.5 kDa) non-immunoglobulin-engineered proteins based on a three-helix bundle domain framework that is based on a 58-amino-acid Z-domain scaffold, derived from one of the IgG-binding domains of staphylococcal protein A and can be engineered for desired target recognition. See e.g., Frejd and Kim. 2017. Exp. Mol. Med. 49 (3):e306; Löfblom J, et al. FEBS Lett. 2010 Jun. 18; 584 (12): 2670-80. doi: 10.1016/j.febslet.2010.04.014. Epub 2010 Apr. 11; and Nygren, P. A. FEBS J. 2008 June; 275 (11): 2668-76.

The term “antibody-like protein scaffolds” or “engineered protein scaffolds” broadly encompasses proteinaceous non-immunoglobulin specific-binding agents, typically obtained by combinatorial engineering (such as site-directed random mutagenesis in combination with phage display or other molecular selection techniques). Usually, such scaffolds are derived from robust and small soluble monomeric proteins (such as Kunitz inhibitors or lipocalins) or from a stably folded extra-membrane domain of a cell surface receptor (such as protein A, fibronectin, or the ankyrin repeat).

Such scaffolds have been extensively reviewed in Binz et al. Engineering novel binding proteins from nonimmunoglobulin domains. Nat Biotechnol 2005, 23:1257-1268; Gebauer and Skerra. Engineered protein scaffolds as next-generation antibody therapeutics. Curr Opin Chem Biol. 2009, 13:245-55; Gill and Damle. Biopharmaceutical drug discovery using novel protein scaffolds. Curr Opin Biotechnol 2006, 17:653-658; Skerra. Engineered protein scaffolds for molecular recognition. J Mol Recognit 2000, 13:167-187; and Skerra. Alternative non-antibody scaffolds for molecular recognition. Curr Opin Biotechnol 2007, 18:295-304; and include without limitation affibodies, based on the Z-domain of staphylococcal protein A, a three-helix bundle of 58 residues providing an interface on two of its alpha-helices (Nygren, Alternative binding proteins: Affibody binding proteins developed from a small three-helix bundle scaffold. FEBS J 2008, 275:2668-2676); engineered Kunitz domains based on a small (ca. 58 residues) and robust, disulfide-crosslinked serine protease inhibitor, typically of human origin (e.g., LACI-D1), which can be engineered for different protease specificities (Nixon and Wood, Engineered protein inhibitors of proteases. Curr Opin Drug Discov Dev 2006, 9:261-268); monobodies or adnectins based on the 10th extracellular domain of human fibronectin III (10Fn3), which adopts an Ig-like beta-sandwich fold (94 residues) with 2-3 exposed loops, but lacks the central disulfide bridge (Koide and Koide, Monobodies: antibody mimics based on the scaffold of the fibronectin type III domain. Methods Mol Biol 2007, 352:95-109); anticalins derived from the lipocalins, a diverse family of eight-stranded beta-barrel proteins (ca. 180 residues) that naturally form binding sites for small ligands by means of four structurally variable loops at the open end, which are abundant in humans, insects, and many other organisms (Skerra, Alternative binding proteins: Anticalins-harnessing the structural plasticity of the lipocalin ligand pocket to engineer novel binding activities. FEBS J 2008, 275:2677-2683); DARPins, designed ankyrin repeat domains (166 residues), which provide a rigid interface arising from typically three repeated beta-turns (Stumpp et al., DARPins: a new generation of protein therapeutics. Drug Discov Today 2008, 13:695-701); avimers (multimerized LDLR-A module) (Silverman et al., Multivalent avimer proteins evolved by exon shuffling of a family of human receptor domains. Nat Biotechnol 2005, 23:1556-1561); and cysteine-rich knottin peptides (Kolmar, Alternative binding proteins: biological activity and therapeutic potential of cystine-knot miniproteins. FEBS J 2008, 275:2684-2690).

In an embodiment, the therapeutic protein is an engineered bifunctional protein, such as degrons, PROTACs, molecular glues, See e.g., Du and Xu et al., Adv. Materials. 33 (48): 2103114 (2021); Modell et al., Cell Chem Biol. 28 (7): 1081-1089 (2021), Sun et al., Signal Transduction and Targeted Therapy, 4:64 (2019); Gao et al., ACS Med Chem Lett. 2020, 11:3, 237-240; Schreiber et al., Cell. 184:3-9 (2021); and Prozillo et al., Biology. 2020. 9 (12): 421.

### Genetic Modifying Agents

In certain embodiments, the one or more modulating agents may be a genetic modifying agent. The genetic modifying agent may comprise a programmable nuclease system (e.g. an RNA-guided system (e.g., CRISPR system, IscB system, or OMEGA system), a zinc finger nuclease system, a TALEN, a meganuclease), an RNAi system, or a combination thereof. In an embodiment, a polynucleotide of the present invention described elsewhere herein can be modified using a genetic modifying agent.

### CRISPR-Cas

In general, a CRISPR-Cas or CRISPR system as used herein and in documents, such as WO 2014/093622 (PCT/US2013/074667), refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or “RNA(s)” as that term is herein used (e.g., RNA(s) to guide Cas, such as Cas9, e.g. CRISPR RNA and transactivating (tracr) RNA or a single guide RNA (sgRNA) (chimeric RNA)), or other sequences and transcripts from a CRISPR locus. In general, a CRISPR system is characterized by elements that promote the formation of a CRISPR complex at the site of a target sequence (also referred to as a protospacer in the context of an endogenous CRISPR system). See, e.g., Shmakov et al. (2015) “Discovery and Functional Characterization of Diverse Class 2 CRISPR-Cas Systems”, Molecular Cell, DOI: dx.doi.org/10.1016/j.molcel.2015.10.008. The term “CRISPR systems” includes any form such as polynucleotides, proteins, and complexes (e.g., RNPs), which are described in greater detail elsewhere herein. The terms “CRISPR-Cas system” and “CRISPR system” are used interchangeably herein.

### Class 1 Systems

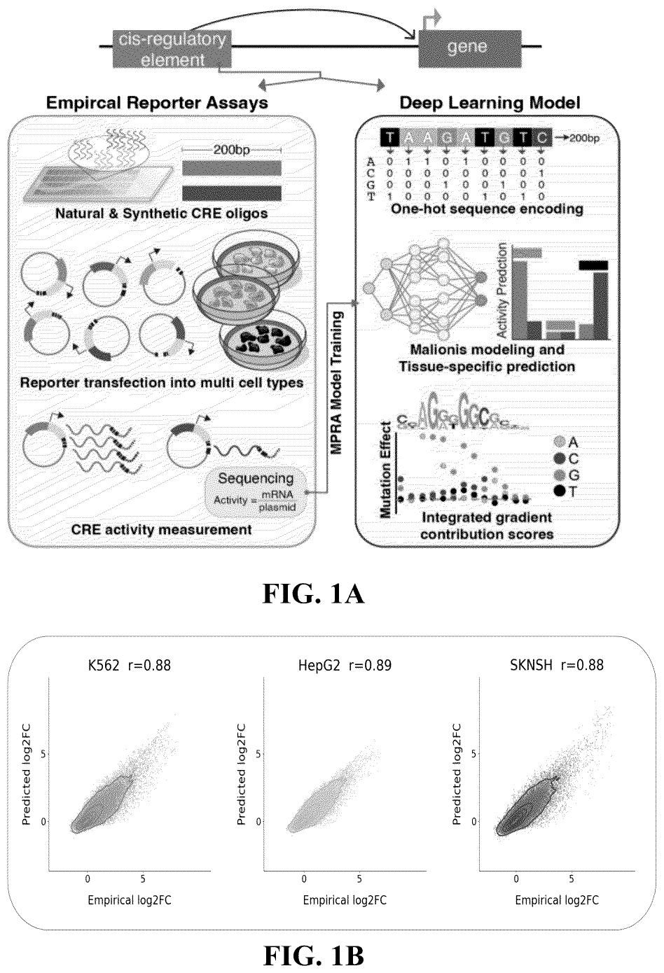

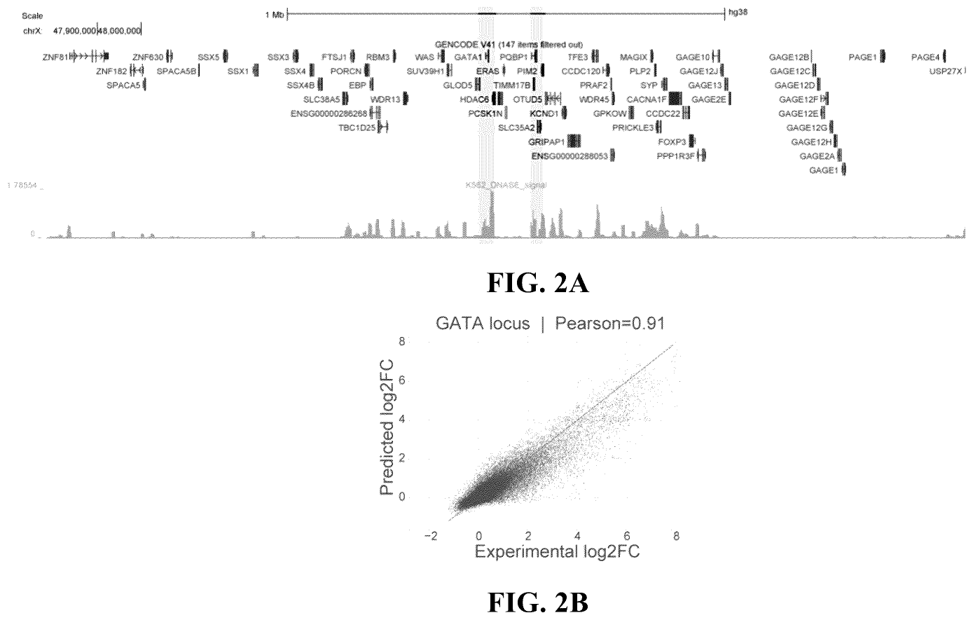

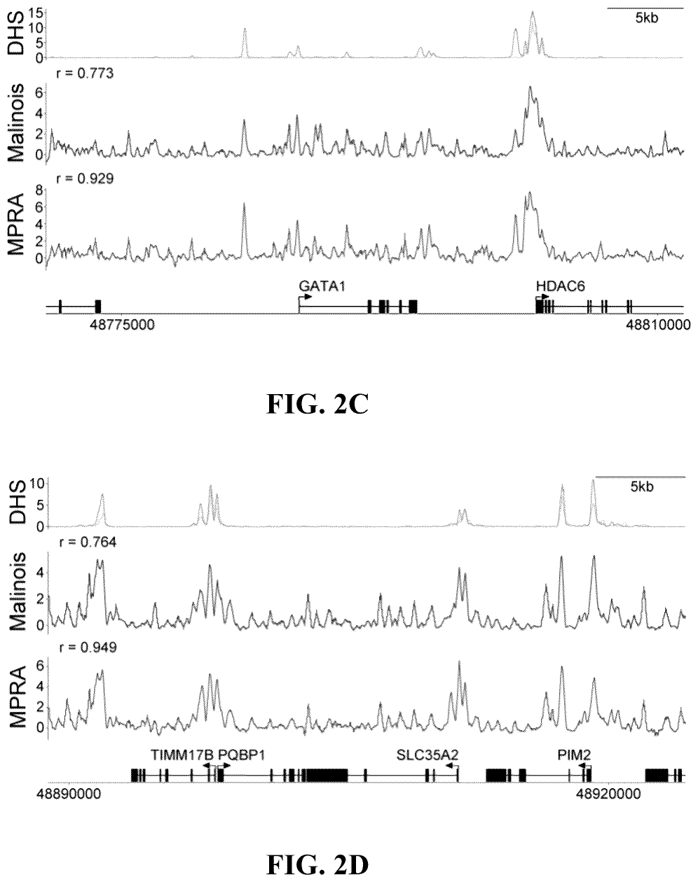

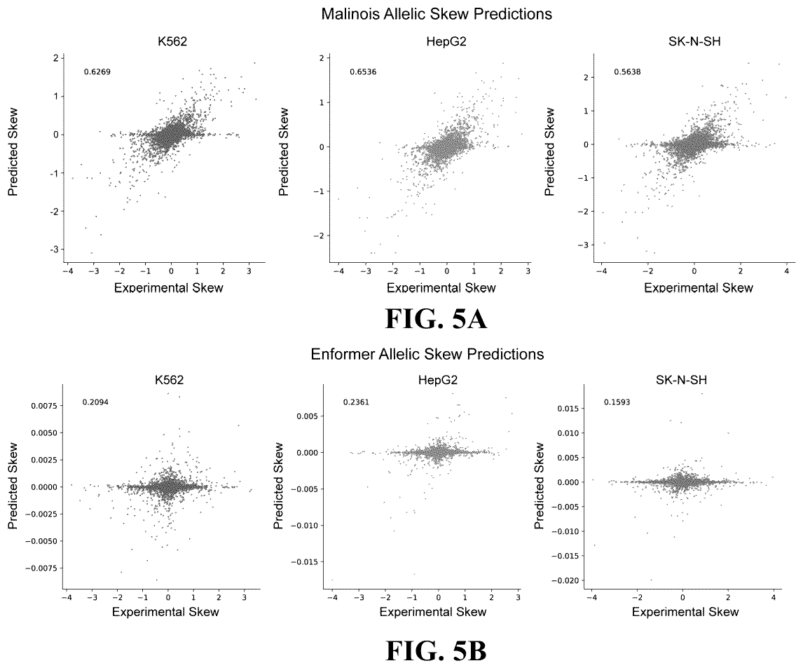

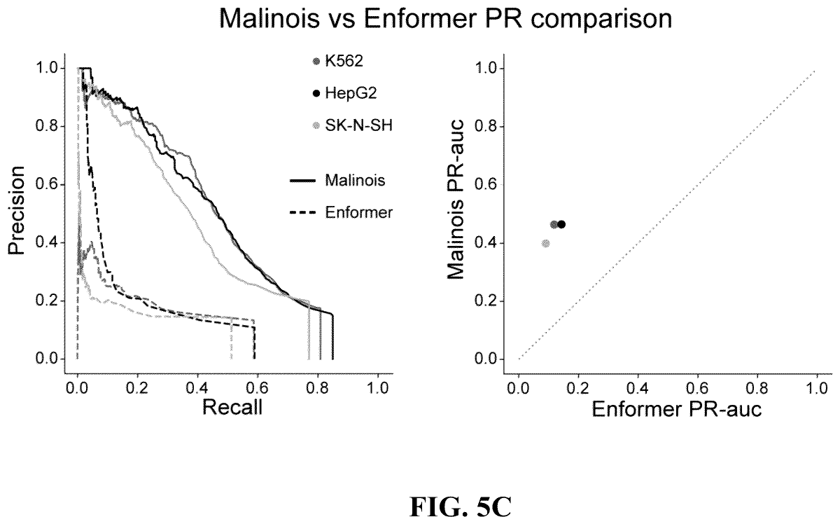

The methods, systems, and tools provided herein may be designed for use with Class 1 CRISPR proteins. In certain example embodiments, the Class 1 system may be Type I, Type III or Type IV Cas proteins as described in Makarova et al. “Evolutionary classification of CRISPR-Cas systems: a burst of class 2 and derived variants” Nature Reviews Microbiology, 18:67-81 (February 2020)., incorporated in its entirety herein by reference, and particularly as described in FIG. 1, p. 326. The Class 1 systems typically use a multi-protein effector complex, which can, In an embodiment, include ancillary proteins, such as one or more proteins in a complex referred to as a CRISPR-associated complex for antiviral defense (Cascade), one or more adaptation proteins (e.g., Cas1, Cas2, RNA nuclease), and/or one or more accessory proteins (e.g. Cas 4, DNA nuclease, Cas3, etc.), CRISPR associated Rossmann fold (CARF) domain-containing proteins, and/or RNA transcriptase. Although Class 1 systems have limited sequence similarity, Class 1 system proteins can be identified by their similar architectures, including one or more Repeat-Associated Mysterious Protein (RAMP) family subunits, e.g., Cas 5, Cas6, Cas7. RAMP proteins are characterized by having one or more RNA recognition motif domains. Large subunits (for example, Cas8 or Cas10) and small subunits (for example, Cas11) are also typical of Class 1 systems. See, e.g., FIGS. 1 and 2. Koonin E V, Makarova K S. 2019 Origins and evolution of CRISPR-Cas systems. Phil. Trans. R. Soc. B 374:20180087, DOI: 10.1098/rstb.2018.0087. In one aspect, Class 1 systems are characterized by the signature protein Cas3. The Cascade in particular Class 1 proteins can comprise a dedicated complex of multiple Cas proteins that binds pre-crRNA and recruits an additional Cas protein, for example, Cas6 or Cas5, which is the nuclease directly responsible for processing pre-crRNA. In one aspect, the Type I CRISPR protein comprises an effector complex with one or more Cas5 subunits and two or more Cas7 subunits. Class 1 subtypes include Type I-A, I-B, I-C, I-U, I-D, I-E, and I-F, Type IV-A and IV-B, IV-C, and Type III-A, III-D, III-B, III-C, III-E, and III-F III-B. See e.g., Marakova et al., Nat. Rev. Microbiol. 18, pages 67-83 (2020). Class 1 systems also include CRISPR-Cas variants, including Type I-A, I-B, I-E, I-F, I-U, and Tye IV variants, which can include variants carried by transposons and plasmids, including versions of subtype I-F encoded by a large family of Tn7-like transposon and smaller groups of Tn7-like transposons that encode similarly degraded subtype I-B systems. Peters et al., PNAS 114 (35) (2017); DOI: 10.1073/pnas.1709035114; see also, Makarova et al, the CRISPR Journal, v. 1, n5, FIG. 5; and Theoretical and Applied Genetics (2022) 135:367-387.

### Class 2 Systems

The compositions, systems, and methods described in greater detail elsewhere herein can be designed and adapted for use with Class 2 CRISPR-Cas systems. Thus, In an embodiment, the CRISPR-Cas system is a Class 2 CRISPR-Cas system. Class 2 systems are distinguished from Class 1 systems in that they have a single, large, multi-domain effector protein. In certain example embodiments, the Class 2 system can be a Type II, Type V, or Type VI system, which are described in Makarova et al. “Evolutionary classification of CRISPR-Cas systems: a burst of class 2 and derived variants” Nature Reviews Microbiology, 18:67-81 (February 2020), incorporated herein by reference. Each type of Class 2 system is further divided into subtypes. See Markova et al. 2020, particularly at FIG. 2. Class 2, Type II systems can be divided into 4 subtypes: II-A, II-B, II-C1, and II-C2. Class 2, Type V systems can be divided into 17 subtypes: V-A, V-B1, V-B2, V-C, V-D, V-E, V-F1, V-F1 (V-U3), V-F2, V-F3, V-G, V-H, V-I, V-K (V-U5), V-U1, V-U2, and V-U4. Class 2, Type VI systems can be divided into 5 subtypes: VI-A, VI-B1, VI-B2, VI-C, and VI-D.

The distinguishing feature of these types is that their effector complexes consist of a single, large, multi-domain protein. Type V systems differ from Type II systems (e.g., Cas9), which contain two nuclear domains (HNH and RuvC) that are each responsible for the cleavage of one strand of the target DNA. The Type V systems (e.g., Cas12) only contain a RuvC-like nuclease domain that cleaves both strands. Type VI (Cas13) are unrelated to the effectors of Type II and V systems and contain two HEPN domains and target RNA. Cas13 proteins also display collateral activity that is triggered by target recognition. Some Type V systems have also been found to possess this collateral activity with single-stranded DNA or RNA. See e.g., Tong et al., Front. Cell. Dev. Biol. 2021, doi.org/10.3389/fcell.2020.622103.

In an embodiment, the Class 2 system is a Type II system. In an embodiment, the Type II CRISPR-Cas system is a II-A CRISPR-Cas system. In an embodiment, the Type II CRISPR-Cas system is a II-B CRISPR-Cas system. In an embodiment, the Type II CRISPR-Cas system is a II-C1 CRISPR-Cas system. In an embodiment, the Type II CRISPR-Cas system is a II-C2 CRISPR-Cas system. In an embodiment, the Type II system is a Cas9 system. In an embodiment, the Type II system includes a Cas9.

In an embodiment, the Class 2 system is a Type V system. In an embodiment, the Type V CRISPR-Cas system is a V-A CRISPR-Cas system. In an embodiment, the Type V CRISPR-Cas system is a V-B1 CRISPR-Cas system. In an embodiment, the Type V CRISPR-Cas system is a V-B2 CRISPR-Cas system. In an embodiment, the Type V CRISPR-Cas system is a V-C CRISPR-Cas system. In an embodiment, the Type V CRISPR-Cas system is a V-D CRISPR-Cas system. In an embodiment, the Type V CRISPR-Cas system is a V-E CRISPR-Cas system. In an embodiment, the Type V CRISPR-Cas system is a V-F1 CRISPR-Cas system. In an embodiment, the Type V CRISPR-Cas system is a V-F1 (V-U3) CRISPR-Cas system. In an embodiment, the Type V CRISPR-Cas system is a V-F2 CRISPR-Cas system. In an embodiment, the Type V CRISPR-Cas system is a V-F3 CRISPR-Cas system. In an embodiment, the Type V CRISPR-Cas system is a V-G CRISPR-Cas system. In an embodiment, the Type V CRISPR-Cas system is a V-H CRISPR-Cas system. In an embodiment, the Type V CRISPR-Cas system is a V-I CRISPR-Cas system. In an embodiment, the Type V CRISPR-Cas system is a V-K (V-U5) CRISPR-Cas system. In an embodiment, the Type V CRISPR-Cas system is a V-U1 CRISPR-Cas system. In an embodiment, the Type V CRISPR-Cas system is a V-U2 CRISPR-Cas system. In an embodiment, the Type V CRISPR-Cas system is a V-U4 CRISPR-Cas system. In an embodiment, the Type V CRISPR-Cas system includes a Cas12a (Cpf1), Cas12b (C2c1), Cas12b2, Cas12c (C2c3), Cas12d (CasY), Cas12e (CasX), Cas12k, Cas14, Cas12f1 (Cas14a), Cas12f2 (Cas14b), Cas12g, Cas12h, Cas12i, C2c4, C2c8, C2c9, C2c10, and/or Cas@.

In an embodiment the Class 2 system is a Type VI system. In an embodiment, the Type VI CRISPR-Cas system is a VI-A CRISPR-Cas system. In an embodiment, the Type VI CRISPR-Cas system is a VI-B1 CRISPR-Cas system. In an embodiment, the Type VI CRISPR-Cas system is a VI-B2 CRISPR-Cas system. In an embodiment, the Type VI CRISPR-Cas system is a VI-C CRISPR-Cas system. In an embodiment, the Type VI CRISPR-Cas system is a VI-D CRISPR-Cas system. In an embodiment, the Type VI CRISPR-Cas system includes a Cas13a (C2c2), Cas13b, Cas13c, and/or Cas13d.

### Guide Molecules

The CRISPR-Cas or Cas-Based system described herein can, In an embodiment, include one or more guide molecules. The terms guide molecule, guide sequence and guide polynucleotide refer to polynucleotides capable of guiding Cas to a target genomic locus and are used interchangeably as in foregoing cited documents such as International Patent Publication No. WO 2014/093622 (PCT/US2013/074667). In general, a guide sequence is any polynucleotide sequence having sufficient complementarity with a target polynucleotide sequence to hybridize with the target sequence and direct sequence-specific binding of a CRISPR complex to the target sequence. The guide molecule can be a polynucleotide.

The ability of a guide sequence (within a nucleic acid-targeting guide RNA) to direct sequence-specific binding of a nucleic acid-targeting complex to a target nucleic acid sequence may be assessed by any suitable assay. For example, the components of a nucleic acid-targeting CRISPR system sufficient to form a nucleic acid-targeting complex, including the guide sequence to be tested, may be provided to a host cell having the corresponding target nucleic acid sequence, such as by transfection with vectors encoding the components of the nucleic acid-targeting complex, followed by an assessment of preferential targeting (e.g., cleavage) within the target nucleic acid sequence, such as by the Surveyor assay (Qui et al. 2004. BioTechniques. 36(4)702-707). Similarly, cleavage of a target nucleic acid sequence may be evaluated in a test tube by providing the target nucleic acid sequence, components of a nucleic acid-targeting complex, including the guide sequence to be tested and a control guide sequence different from the test guide sequence, and comparing binding or rate of cleavage at the target sequence between the test and control guide sequence reactions. Other assays are possible and will occur to those skilled in the art.

In an embodiment, the guide molecule is an RNA. The guide molecule(s) (also referred to interchangeably herein as guide polynucleotide and guide sequence) that are included in the CRISPR-Cas or Cas-based system can be any polynucleotide sequence having sufficient complementarity with a target nucleic acid sequence to hybridize with the target nucleic acid sequence and direct sequence-specific binding of a nucleic acid-targeting complex to the target nucleic acid sequence. In an embodiment, the degree of complementarity, when optimally aligned using a suitable alignment algorithm, can be about or more than about 50%, 60%, 75%, 80%, 85%, 90%, 95%, 97.5%, 99%, or more. Optimal alignment may be determined with the use of any suitable algorithm for aligning sequences, non-limiting examples of which include the Smith-Waterman algorithm, the Needleman-Wunsch algorithm, algorithms based on the Burrows-Wheeler Transform (e.g., the Burrows-Wheeler Aligner), ClustalW, Clustal X, BLAT, Novoalign (Novocraft Technologies; available at novocraft.com), ELAND (Illumina, San Diego, CA), SOAP (available at soap.genomics.org.cn), and Maq (available at maq.sourceforge.net).

A guide sequence, and hence a nucleic acid-targeting guide, may be selected to target any target nucleic acid sequence. The target sequence may be DNA. The target sequence may be any RNA sequence. In an embodiment, the target sequence may be a sequence within an RNA molecule selected from the group consisting of messenger RNA (mRNA), pre-mRNA, ribosomal RNA (rRNA), transfer RNA (tRNA), micro-RNA (miRNA), small interfering RNA (siRNA), small nuclear RNA (snRNA), small nucleolar RNA (snoRNA), double-stranded RNA (dsRNA), non-coding RNA (ncRNA), long non-coding RNA (lncRNA), and small cytoplasmaic RNA (scRNA). In some preferred embodiments, the target sequence may be a sequence within an RNA molecule selected from the group consisting of mRNA, pre-mRNA, and rRNA. In some preferred embodiments, the target sequence may be a sequence within an RNA molecule selected from the group consisting of ncRNA, and lncRNA. In some more preferred embodiments, the target sequence may be a sequence within an mRNA molecule or a pre-mRNA molecule.

In an embodiment, a nucleic acid-targeting guide is selected to reduce the degree of secondary structure within the nucleic acid-targeting guide. In an embodiment, about or less than about 75%, 50%, 40%, 30%, 25%, 20%, 15%, 10%, 5%, 1%, or fewer of the nucleotides of the nucleic acid-targeting guide participate in self-complementary base pairing when optimally folded. Optimal folding may be determined by any suitable polynucleotide folding algorithm. Some programs are based on calculating the minimal Gibbs free energy. An example of one such algorithm is mFold, as described by Zuker and Stiegler (Nucleic Acids Res. 9 (1981), 133-148). Another example folding algorithm is the online webserver RNAfold, developed at Institute for Theoretical Chemistry at the University of Vienna, using the centroid structure prediction algorithm (see e.g., A. R. Gruber et al., 2008, Cell 106 (1): 23-24; and PA Carr and GM Church, 2009, Nature Biotechnology 27 (12): 1151-62).

In certain embodiments, a guide RNA or CRISPR RNA (crRNA) may comprise, consist essentially of, or consist of a direct repeat (DR) sequence and a guide sequence or spacer sequence. In certain embodiments, the guide RNA or crRNA may comprise, consist essentially of, or consist of a direct repeat sequence fused or linked to a guide sequence or spacer sequence. In certain embodiments, the direct repeat sequence may be located upstream (i.e., 5′) from the guide sequence or spacer sequence. In other embodiments, the direct repeat sequence may be located downstream (i.e., 3′) from the guide sequence or spacer sequence.

In certain embodiments, the crRNA comprises a stem-loop, preferably a single stem-loop. In certain embodiments, the direct repeat sequence forms a stem-loop, preferably a single stem-loop.

In certain embodiments, the spacer length of the guide RNA is from 15 to 35 nucelotides (nt). In certain embodiments, the spacer length of the guide RNA is at least 15 nt. In certain embodiments, the spacer length is from 15 to 17 nt, e.g., 15, 16, or 17 nt, from 17 to 20 nt, e.g., 17, 18, 19, or 20 nt, from 20 to 24 nt, e.g., 20, 21, 22, 23, or 24 nt, from 23 to 25 nt, e.g., 23, 24, or 25 nt, from 24 to 27 nt, e.g., 24, 25, 26, or 27 nt, from 27 to 30 nt, e.g., 27, 28, 29, or 30 nt, from 30 to 35 nt, e.g., 30, 31, 32, 33, 34, or 35 nt, or 35 nt or longer.

The “tracrRNA” sequence or analogous terms includes any polynucleotide sequence that has sufficient complementarity with a crRNA sequence to hybridize. In an embodiment, the degree of complementarity between the tracrRNA sequence and crRNA sequence along the length of the shorter of the two when optimally aligned is about or more than about 25%, 30%, 40%, 50%, 60%, 70%, 80%, 90%, 95%, 97.5%, 99%, or higher. In an embodiment, the tracr sequence is about or more than about 5, 6, 7, 8, 9, 10, 11, 12, 13, 14, 15, 16, 17, 18, 19, 20, 25, 30, 40, 50, or more nucleotides in length. In an embodiment, the tracr sequence and crRNA sequence are contained within a single transcript, such that hybridization between the two produces a transcript having a secondary structure, such as a hairpin.

In general, the degree of complementarity is with reference to the optimal alignment of the guide sequence and tracr sequence, along the length of the shorter of the two sequences. Optimal alignment may be determined by any suitable alignment algorithm and may further account for secondary structures, such as self-complementarity within either the guide sequence or tracr sequence. In an embodiment, the degree of complementarity between the tracr sequence and guide sequence along the length of the shorter of the two when optimally aligned is about or more than about 25%, 30%, 40%, 50%, 60%, 70%, 80%, 90%, 95%, 97.5%, 99%, or higher.

In an embodiment, the degree of complementarity between a guide sequence and its corresponding target sequence can be about or more than about 50%, 60%, 75%, 80%, 85%, 90%, 95%, 97.5%, 99%, or 100%. In an embodiment, a guide RNA or sgRNA can be about or more than about 5, 10, 11, 12, 13, 14, 15, 16, 17, 18, 19, 20, 21, 22, 23, 24, 25, 26, 27, 28, 29, 30, 35, 40, 45, 50, 75, 76, 77, 78, 79, 80, 81, 82, 83, 84, 85, 86, 87, 88, 89, 90, 91, 92, 93, 94, 95, 96, 97, 98, 99, 100 or more nucleotides in length. In an embodiment, a guide RNA or sgRNA can be less than about 100, 75, 50, 45, 40, 35, 30, 25, 20, 15, 12, or fewer nucleotides in length; and a tracr RNA can be 30 or 50 nucleotides in length. In an embodiment, the degree of complementarity between a guide sequence and its corresponding target sequence is greater than 94.5% or 95% or 95.5% or 96% or 96.5% or 97% or 97.5% or 98% or 98.5% or 99% or 99.5% or 99.9%, or 100%. Off target is less than 100% or 99.9% or 99.5% or 99% or 99% or 98.5% or 98% or 97.5% or 97% or 96.5% or 96% or 95.5% or 95% or 94.5% or 94% or 93% or 92% or 91% or 90% or 89% or 88% or 87% or 86% or 85% or 84% or 83% or 82% or 81% or 80% complementarity between the sequence and the guide, with it being advantageous that there is 100% or 99.9% or 99.5% or 99% or 99% or 98.5% or 98% or 97.5% or 97% or 96.5% or 96% or 95.5% or 95% or 94.5% complementarity between the sequence and the guide.

In an embodiment according to the invention, the guide RNA (capable of guiding Cas to a target locus) may comprise (1) a guide sequence capable of hybridizing to a genomic target locus in the eukaryotic cell; (2) a tracr sequence; and (3) a tracr mate sequence. All (1) to (3) may reside in a single RNA, i.e., an sgRNA (arranged in a 5′ to 3′ orientation), or the tracr RNA may be a different RNA than the RNA containing the guide and tracr mate sequence. The tracr hybridizes to the tracr mate sequence and directs the CRISPR/Cas complex to the target sequence. Where the tracr RNA is on a different RNA than the RNA containing the guide and tracr mate sequence, the length of each RNA may be optimized to be shortened from their respective native lengths, and each may be independently chemically modified to protect from degradation by cellular ribonucleases or otherwise increase stability.

Many modifications to guide sequences are known in the art and are further contemplated within the context of this invention. Various modifications may be used to increase the specificity of binding to the target sequence and/or increase the activity of the Cas protein and/or reduce off-target effects. Example guide sequence modifications are described in International Patent Application No. PCT US2019/045582, specifically paragraphs [0178][0333]. which is incorporated herein by reference.

### Target Sequences, PAMs, and PFSs

In the context of the formation of a CRISPR complex, “target sequence” refers to a sequence to which a guide sequence is designed to have complementarity, where hybridization between a target sequence and a guide sequence promotes the formation of a CRISPR complex. It will be appreciated that “CRISPR complex” generally refers to a Cas complexed with a guide RNA and optionally a target polynucleotide, and/or other molecules involved in activity of the CRISPR-Cas system. Such a term includes RNPs formed of a Cas protein complexed with a gRNA and those otherwise formed. A target sequence may comprise RNA polynucleotides. The term “target RNA” refers to an RNA polynucleotide being or comprising the target sequence. In other words, the target polynucleotide can be a polynucleotide or a part of a polynucleotide to which a part of the guide sequence is designed to have complementarity with and to which the effector function mediated by the complex comprising the CRISPR effector protein and a guide molecule is to be directed. In an embodiment, a target sequence is located in the nucleus or cytosol of a cell.

The guide sequence can specifically bind a target sequence in a target polynucleotide. The target polynucleotide may be DNA. The target polynucleotide may be RNA. The target polynucleotide can have one or more (e.g., 1, 2, 3, 4, 5, 6, 7, 8, 9, 10, etc. or more) target sequences. The target polynucleotide can be on a vector. The target polynucleotide can be genomic DNA. The target polynucleotide can be episomal. Other forms of the target polynucleotide are described elsewhere herein.

The target sequence may be DNA. The target sequence may be any RNA sequence. In an embodiment, the target sequence may be a sequence within an RNA molecule selected from the group consisting of messenger RNA (mRNA), pre-mRNA, ribosomal RNA (rRNA), transfer RNA (tRNA), micro-RNA (miRNA), small interfering RNA (siRNA), small nuclear RNA (snRNA), small nucleolar RNA (snoRNA), double-stranded RNA (dsRNA), non-coding RNA (ncRNA), long non-coding RNA (lncRNA), and small cytoplasmatic RNA (scRNA). In some preferred embodiments, the target sequence (also referred to herein as a target polynucleotide) may be a sequence within an RNA molecule selected from the group consisting of mRNA, pre-mRNA, and rRNA. In some preferred embodiments, the target sequence may be a sequence within an RNA molecule selected from the group consisting of ncRNA, and lncRNA. In some more preferred embodiments, the target sequence may be a sequence within an mRNA molecule or a pre-mRNA molecule.

### PAM and PES Elements

PAM (protospacer adjacent motif) elements are sequences that can be recognized and bound by Cas proteins. Cas proteins/effector complexes can then unwind the dsDNA at a position adjacent to the PAM element. It will be appreciated that RNA-targeting Cas proteins and systems do not require PAM sequences (Marraffini et al. 2010. Nature. 463:568-571). Instead, many rely on PFSs (protospacer flanking sequence or site), which are discussed elsewhere herein. In certain embodiments, the target sequence should be associated with a PAM or PFS, that is, a short sequence recognized by the CRISPR complex. Depending on the nature of the CRISPR-Cas protein, the target sequence should be selected, such that its complementary sequence in the DNA duplex (also referred to herein as the non-target sequence) is upstream or downstream of the PAM. In an embodiment, the complementary sequence of the target sequence is downstream (3′ of the PAM) or upstream (5′ of the PAM). The precise sequence and length requirements for the PAM differ depending on the Cas protein used, but PAMs are typically 2-5 base pair sequences adjacent to the protospacer (that is, the target sequence). Examples of the natural PAM sequences for different Cas proteins are provided herein below and the skilled person will be able to identify further PAM sequences for use with a given Cas protein.

The ability to recognize different PAM sequences depends on the Cas polypeptide(s) included in the system. See e.g., Gleditzsch et al. 2019. RNA Biology. 16 (4): 504-517. Table 2 (from Gleditzsch et al. 2019) below shows several Cas polypeptides and the PAM sequence they recognize.

TABLE 2
Example PAM Sequences

| Cas Protein | PAM Sequence |
|---|---|
| SpCas9 | NGG/NRG |
| SaCas9 | NGRRT or NGRRN |
| NmeCas9 | NNNNGATT |
| CjCas9 | NNNNRYAC |
| StCas9 | NNAGAAW |
| Cas12a (Cpf1) (including | TTTV |
| LbCpf1 and AsCpf1) | |
| Cas12b (C2c1) | TTT, TTA, and TTC |
| Cas12c (C2c3) | TA |
| Cas12d (CasY) | TA |
| Cas12e (CasX) | TTCN |

In an embodiment, the CRISPR effector protein may recognize a 3′ PAM. In certain embodiments, the CRISPR effector protein may recognize a 3′ PAM which is 5′H, wherein H is A, C or U. In an embodiment, the CRISPR effector protein may recognize a 5′ PAM.

Further, engineering of the PAM Interacting (PI) domain on the Cas protein may allow the programming of PAM specificity to improve target site recognition fidelity, and increase the versatility of the CRISPR-Cas protein, for example as described for Cas9 in Kleinstiver B P et al. Engineered CRISPR-Cas9 nucleases with altered PAM specificities. Nature. 2015 Jul. 23; 523 (7561): 481-5. doi: 10.1038/nature14592. As further detailed herein, the skilled person will understand that Cas12 proteins may be modified analogously. Gao et al, “Engineered Cpf1 Enzymes with Altered PAM Specificities,” bioRxiv 091611; doi: http://dx.doi.org/10.1101/091611 (Dec. 4, 2016) and Gao et al. Nat. Biotechnol. 35, 789-792 (2017). Doench et al. Nat Biotechnol. 2016 February; 34 (2): 184-191 created a pool of sgRNAs, tiling across all possible target sites of a panel of six endogenous mice and three endogenous human genes and quantitatively assessed their ability to produce null alleles of their target gene by antibody staining and flow cytometry. The authors showed that optimization of the PAM improved activity and also provided an online tool for designing sgRNAs. In an embodiment, the CRISPR-Cas system recognizes such an optimized PAM.

PAM sequences can be identified in a polynucleotide using appropriate design tools, which are commercially available as well as online. Such freely available tools include, but are not limited to, CRISPRFinder and CRISPRTarget. Mojica et al. 2009. Microbiol. 155 (Pt. 3): 733-740; Atschul et al. 1990. J. Mol. Biol. 215:403-410; Biswass et al. 2013 RNA Biol. 10:817-827; and Grissa et al. 2007. Nucleic Acid Res. 35: W52-57. Experimental approaches to PAM identification can include, but are not limited to, plasmid depletion assays (Jiang et al. 2013. Nat. Biotechnol. 31:233-239; Esvelt et al. 2013. Nat. Methods. 10:1116-1121; Kleinstiver et al. 2015. Nature. 523:481-485), screening by a high-throughput in vivo model called PAM-SCNAR (Pattanayak et al. 2013. Nat. Biotechnol. 31:839-843 and Leenay et al. 2016. Mol. Cell. 16:253), and negative screening (Zetsche et al. 2015. Cell. 163:759-771).

As previously mentioned, CRISPR-Cas systems that target RNA do not typically rely on PAM sequences. Instead, such systems typically recognize protospacer flanking sites (PFSs) instead of PAMs. Thus, Type VI CRISPR-Cas systems typically recognize protospacer flanking sites (PFSs) instead of PAMs. PFSs represent an analog to PAMs for RNA targets. Type VI CRISPR-Cas systems employ a Cas13. Some Cas13 proteins analyzed to date, such as Cas13a (C2c2) identified from Leptotrichia shahii (LshCas13a) have a specific discrimination against G at 3′end of the target RNA. The presence of a C at the corresponding crRNA repeat site can indicate that nucleotide pairing at this position is rejected. However, some Cas13 proteins (e.g., LwaCas13a and PspCas13b) do not seem to have a PFS preference. See e.g., Gleditzsch et al. 2019. RNA Biology. 16 (4): 504-517.

Some Type VI proteins, such as subtype B, have 5′-recognition of D (G, T, A) and a 3′-motif requirement of NAN or NNA. One example is the Cas13b protein identified in Bergeyella zoohelcum (BzCas13b). See e.g., Gleditzsch et al. 2019. RNA Biology. 16 (4): 504-517.

Overall Type VI CRISPR-Cas systems appear to have less restrictive rules for substrate (e.g., target sequence) recognition than those that target DNA (e.g., Type V and type II).

### Specialized Cas-Based Systems

In an embodiment, the system is a Cas-based system that is capable of performing a specialized function or activity. For example, the Cas protein may be fused, operably coupled to, or otherwise associated with one or more functional domains. In certain example embodiments, the Cas protein may be a catalytically dead Cas protein (“dCas”) and/or have nickase activity. A nickase is a Cas protein that cuts only one strand of a double-stranded target. In such embodiments, the dCas or nickase provides a sequence-specific targeting functionality that positions the functional domain to or proximate to a target sequence. Example functional domains that may be fused to, operably coupled to, or otherwise associated with a Cas protein can be or include, but are not limited to a nuclear localization signal (NLS) domain, a nuclear export signal (NES) domain, a translational activation domain, a transcriptional activation domain (e.g. VP64, p65, MyoD1, HSF1, RTA, and SET7/9), a translation initiation domain, a transcriptional repression domain (e.g., a KRAB domain, NuE domain, NcoR domain, and a SID domain such as a SID4X domain), a nuclease domain (e.g., FokI), a histone modification domain (e.g., a histone acetyltransferase), a light-inducible/controllable domain, a chemically-inducible/controllable domain, a transposase domain, a homologous recombination machinery domain, a recombinase domain, an integrase domain, a deaminase domain, and combinations thereof. Methods for generating catalytically dead Cas9 or a nickase Cas9 (WO 2014/204725, Ran et al. Cell. 2013 Sep. 12; 154(6):1380-1389), Cas12 (Liu et al. Nature Communications, 8, 2095 (2017), and Cas13 (International Patent Publication Nos. WO 2019/005884 and WO2019/060746) are known in the art and incorporated herein by reference.

In an embodiment, the functional domains can have one or more of the following activities: methylase activity, demethylase activity, translation activation activity, translation initiation activity, translation repression activity, transcription activation activity, transcription repression activity, transcription release factor activity, histone modification activity, nuclease activity, single-strand RNA cleavage activity, double-strand RNA cleavage activity, single-strand DNA cleavage activity, double-strand DNA cleavage activity, molecular switch activity, chemical inducibility, light inducibility, and nucleic acid binding activity. In an embodiment, one or more functional domains may comprise epitope tags or reporters. Non-limiting examples of epitope tags include histidine (His) tags, V5 tags, FLAG tags, influenza hemagglutinin (HA) tags, Myc tags, VSV-G tags, and thioredoxin (Trx) tags. Examples of reporters include, but are not limited to, glutathione-S-transferase (GST), horseradish peroxidase (HRP), chloramphenicol acetyltransferase (CAT) beta-galactosidase, beta-glucuronidase, luciferase, green fluorescent protein (GFP), HcRed, DsRed, cyan fluorescent protein (CFP), yellow fluorescent protein (YFP), and auto-fluorescent proteins including blue fluorescent protein (BFP) and mCherry.

One or more functional domain(s) may be positioned at, near, in between, and/or in proximity to a terminus of the effector protein (e.g., a Cas protein). In embodiments having two or more functional domains, each of the two can be positioned at or near or in proximity to a terminus of the effector protein (e.g., a Cas protein). In an embodiment, such as those where the functional domain is operably coupled to the effector protein, one or more functional domains can be tethered or linked via a suitable linker (including, but not limited to, GlySer linkers) to the effector protein (e.g., a Cas protein). When there is more than one functional domain, the functional domains can be the same or different. In an embodiment, all the functional domains are the same. In an embodiment, all of the functional domains are different from each other. In an embodiment, at least two of the functional domains are different from each other. In an embodiment, at least two of the functional domains are the same as each other.

Other suitable functional domains can be found, for example, in International Patent Publication No. WO 2019/018423.

### Split CRISPR-Cas Systems

In an embodiment, the CRISPR-Cas system is a split CRISPR-Cas system. See e.g., Zetsche et al., 2015. Nat. Biotechnol. 33 (2): 139-142 and International Patent Publication WO 2019/018423, the compositions and techniques of which can be used in and/or adapted for use with the present invention. Split CRISPR-Cas proteins are set forth herein and in documents incorporated herein by reference in further detail elsewhere herein. In certain embodiments, each part of a split CRISPR protein is attached to a member of a specific binding pair, and when bound with each other, the members of the specific binding pair maintain the parts of the CRISPR protein in proximity. In certain embodiments, each part of a split CRISPR protein is associated with an inducible binding pair. An inducible binding pair is one which is capable of being switched “on” or “off” by a protein or small molecule that binds to both members of the inducible binding pair. In an embodiment, CRISPR proteins may be preferably split between domains, leaving domains intact. In particular embodiments, said Cas split domains (e.g., RuvC and HNH domains in the case of Cas9) can be simultaneously or sequentially introduced into the cell such that said split Cas domain(s) process the target nucleic acid sequence in the cell. The reduced size of the split Cas compared to the wild-type Cas allows other methods of delivery of the systems to the cells, such as the use of cell-penetrating peptides as described herein.

### DNA and RNA Base Editing

In an embodiment, a polynucleotide can be modified using a base editing system. In an embodiment, a Cas protein is connected or fused to a nucleotide deaminase. Thus, In an embodiment, the Cas-based system can be a base editing system. As used herein, “base editing” refers generally to the process of polynucleotide modification via a CRISPR-Cas-based or Cas-based system that does not include excising nucleotides to make the modification. Base editing can convert base pairs at precise locations without generating excess undesired editing byproducts that can be made using traditional CRISPR-Cas systems.

In certain example embodiments, the nucleotide deaminase may be a DNA base editor used in combination with a DNA binding Cas protein such as, but not limited to, Class 2 Type II and Type V systems. Two classes of DNA base editors are generally known: cytosine base editors (CBEs) and adenine base editors (ABEs). CBEs convert a C·G base pair into a T. A base pair (Komor et al. 2016. Nature. 533:420-424; Nishida et al. 2016. Science. 353; and Li et al. Nat. Biotech. 36:324-327) and ABEs convert an A·T base pair to a G·C base pair. Collectively, CBEs and ABEs can mediate all four possible transition mutations (C to T, A to G, T to C, and G to A). View Rees and Liu. 2018. Nat. Rev. Genet. 19 (12): 770-788, particularly at FIGS. 1b, 2a-2c, 3a-3f, and Table 1. In an embodiment, the base editing system includes a CBE and/or an ABE. In an embodiment, a base editor can modify a polynucleotide. See e.g., Rees and Liu. 2018. Nat. Rev. Gent. 19 (12): 770-788. Base editors also generally do not need a DNA donor template and/or rely on homology-directed repair. Komor et al. 2016. Nature. 533:420-424; Nishida et al. 2016. Science. 353; and Gaudeli et al. 2017. Nature. 551:464-471. Upon binding to a target locus in the DNA, base pairing between the guide RNA of the system and the target DNA strand leads to displacement of a small segment of ssDNA in an “R-loop”. View Nishimasu et al. 2014. Cell. 156:935-949, Lapinaite et al., Science. 369 (6503): 566-572 (2020). DNA bases within the ssDNA bubble are modified by the enzyme component, such as a deaminase. In some systems, the catalytically disabled Cas protein can be a variant or a modified Cas with nickase functionality and can generate a nick in the non-edited DNA strand to induce cells to repair the non-edited strand using the edited strand as a template. Komor et al. 2016. Nature. 533:420-424; Nishida et al. 2016. Science. 353; and Gaudeli et al. 2017. Nature. 551:464-471.

Other Example Type V base editing systems are described in International Patent Publication Nos. WO 2018/213708, WO 2018/213726, and International Patent Applications No. PCT/US2018/067207, PCT/US2018/067225, and PCT/US2018/067307, each of which is incorporated herein by reference.

In certain example embodiments, the base editing system may be an RNA base editing system. As with DNA base editors, a nucleotide deaminase capable of converting nucleotide bases may be fused to a Cas protein. However, in these embodiments, the Cas protein will need to be capable of binding RNA. Example RNA binding Cas proteins include, but are not limited to, RNA-binding Cas9s such as Francisella novicida Cas9 (“FnCas9”), Class 2 Type VI Cas systems, and Cas7-11 (see e.g., Özcan et al., Nature. 597:720-725 (2021)). The nucleotide deaminase may be a cytidine deaminase or an adenosine deaminase, or an adenosine deaminase engineered to have cytidine deaminase activity. In certain example embodiments, the RNA base editor may be used to delete or introduce a post-translational modification site in the expressed mRNA. In contrast to DNA base editors, whose edits are permanent in the modified cell, RNA base editors can provide edits where finer, temporal control may be needed, for example in modulating a particular immune response. Example Type VI RNA-base editing systems are described in Cox et al. 2017. Science 358:1019-1027, International Patent Publication Nos. WO 2019/005884, WO 2019/005886, and WO 2019/071048, and International Patent Application Nos. PCT/US20018/05179 and PCT/US2018/067207, which are incorporated herein by reference. An example FnCas9 system that may be adapted for RNA base editing purposes is described in International Patent Publication No. WO 2016/106236, which is incorporated herein by reference.

An example method for delivery of base-editing systems, including use of a split-intein approach to divide CBE and ABE into reconstitutable halves, is described in Levy et al. Nature Biomedical Engineering doi.org/10.1038/s41441-019-0505-5 (2019), which is incorporated herein by reference.

In an embodiment, the base editor is inhibited by an engineered Acr delivery system or an Acr thereof. In an embodiment, the engineered Acr delivery system of the present invention or an Acr thereof reduces the off-target effects of a base editor system. See e.g., Cells 2020, 9, 1786; doi: 10.3390/cells9081786.

### Prime Editors

In an embodiment, a polynucleotide can be modified using a prime editing system. See e.g. Anzalone et al. 2019. Nature. 576:149-157. Like base editing systems, prime editing systems can be capable of targeted modification of a polynucleotide without generating double-stranded breaks and does not require donor templates. Further, prime editing systems can be capable of all 12 possible combinations of transition and transversion mutations (i.e., A to C, A to T, A to G, C to A, C to T, C to G, T to A, T to G, T to C, G to A, G to T, G to C). Prime editing can operate via a “search-and-replace” methodology and can mediate targeted insertions, deletions, all 12 possible base-to-base conversions and combinations thereof. Generally, a prime editing system, as exemplified by PE1, PE2, and PE3 (Id.), can include a reverse transcriptase fused or otherwise coupled or associated with an RNA-programmable nickase and a prime-editing extended guide RNA (pegRNA) to facilitate direct copying of genetic information from the extension on the pegRNA into the target polynucleotide. Embodiments that can be used with the present invention include these and variants thereof. Prime editing can have the advantage of lower off-target activity than traditional CRISPR-Cas systems along with few byproducts and greater or similar efficiency as compared to traditional CRISPR-Cas systems.

In an embodiment, the prime editing guide molecule can specify both the target polynucleotide information (e.g., sequence) and contain a new polynucleotide cargo that replaces target polynucleotides. To initiate transfer from the guide molecule to the target polynucleotide, the PE system can nick the target polynucleotide at a target side to expose a 3′hydroxyl group, which can prime reverse transcription of an edit-encoding extension region of the guide molecule (e.g., a prime editing guide molecule or peg guide molecule) directly into the target site in the target polynucleotide. See e.g., Anzalone et al. 2019. Nature. 576:149-157, particularly at FIGS. 1b, 1c, related discussion, and Supplementary discussion.

In an embodiment, a prime editing system can be composed of a Cas polypeptide having nickase activity, a reverse transcriptase, and a prime editing guide molecule. The Cas polypeptide can lack nuclease activity. The guide molecule can include a target binding sequence as well as a primer binding sequence and a template containing the edited polynucleotide sequence. The guide molecule, Cas polypeptide, and/or reverse transcriptase can be coupled together or otherwise associate with each other to form an effector complex and edit a target sequence. In an embodiment, the Cas polypeptide is a Class 2, Type V or Type II Cas polypeptide. In an embodiment, the Cas polypeptide is a Cas9 polypeptide (e.g., is a Cas9 nickase). In an embodiment, the Cas polypeptide is fused to the reverse transcriptase. In an embodiment, the Cas polypeptide is linked to the reverse transcriptase.

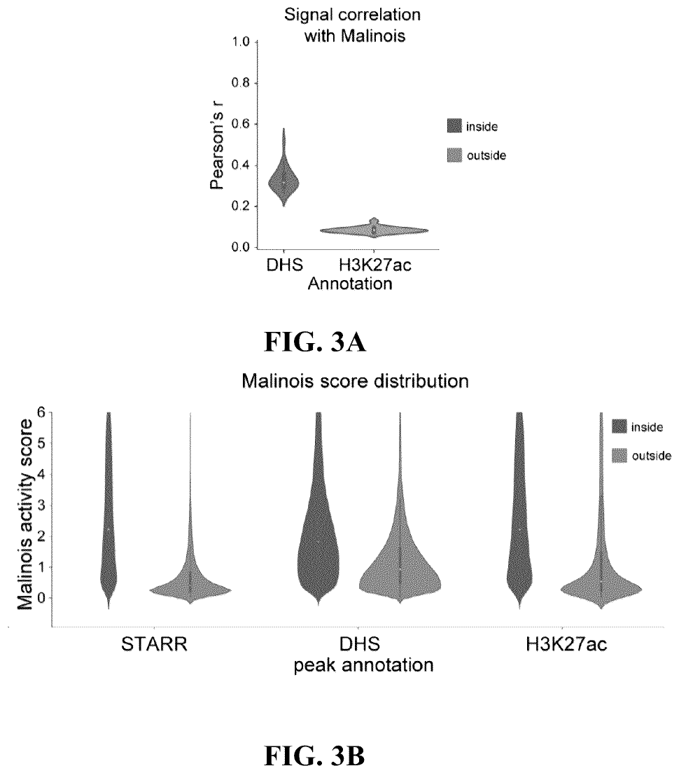

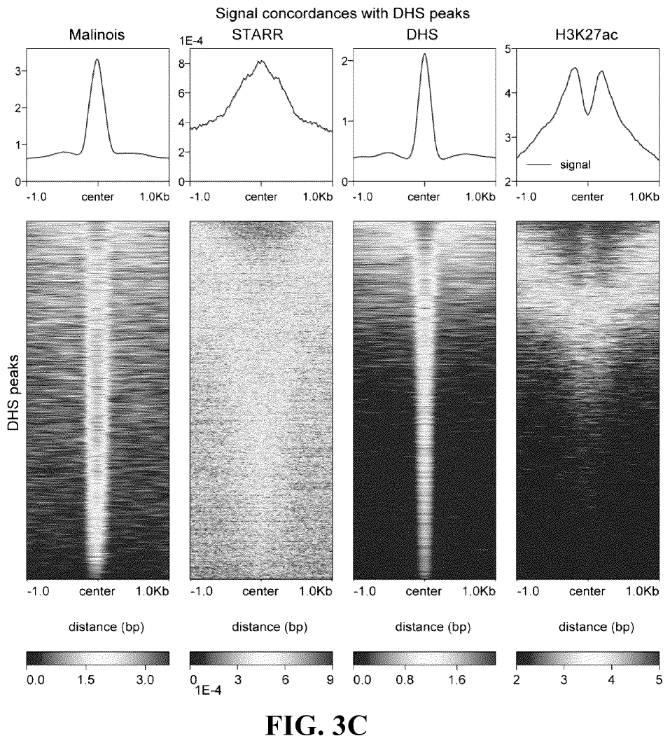

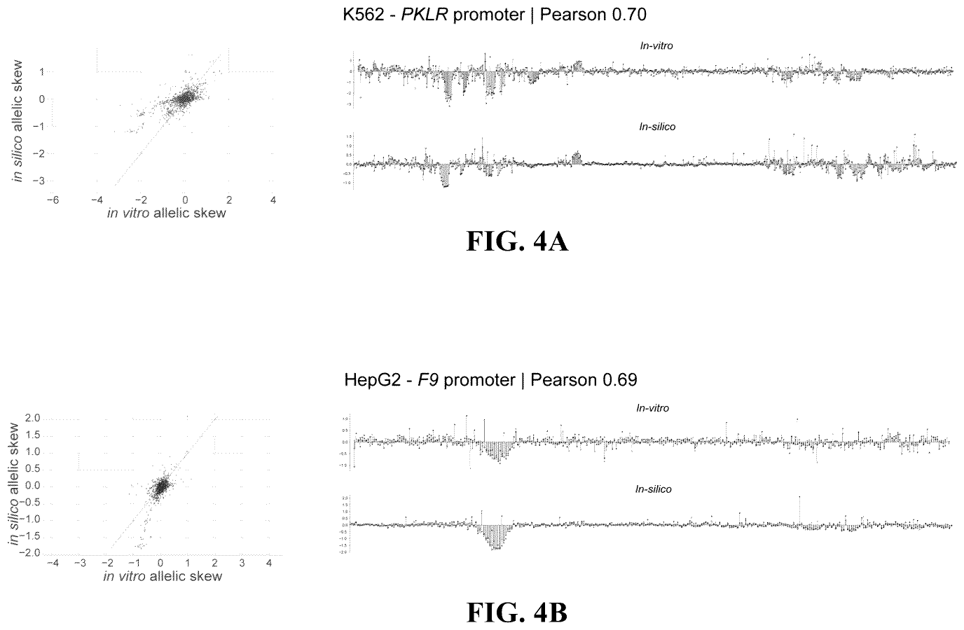

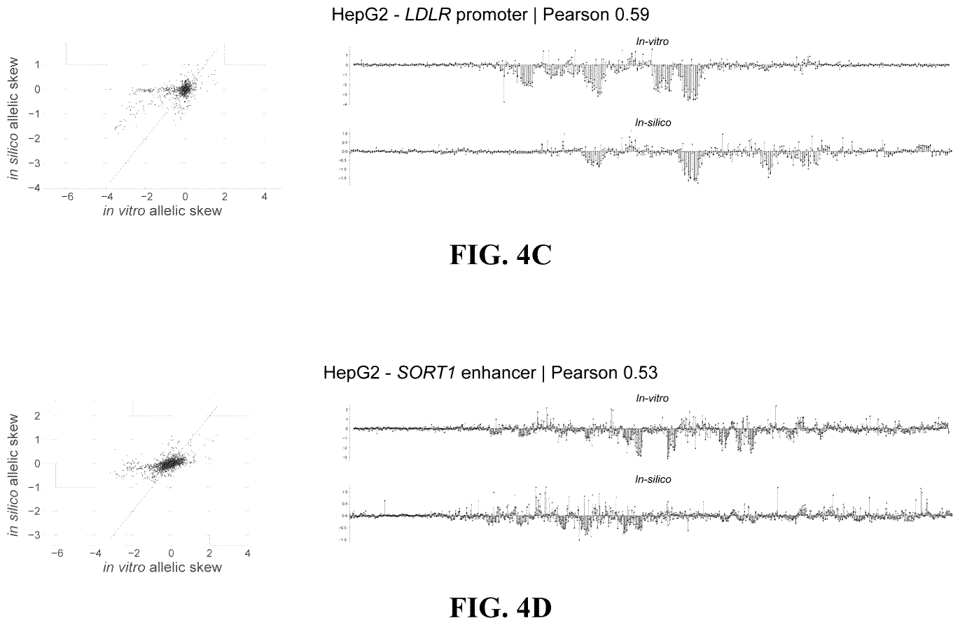

In an embodiment, the prime editing system can be a PE1 system or variant thereof, a PE2 system or variant thereof, or a PE3 (e.g. PE3, PE3b) system. See e.g., Anzalone et al. 2019. Nature. 576:149-157, particularly at pgs. 2-3, FIGS. 2a, 3a-3f, 4a-4b, Extended data FIGS. 3a-3b, 4,

The peg guide molecule can be about 10 to about 200 or more nucleotides in length, such as 10 to/or 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, or 200 or more nucleotides in length. Optimization of the peg guide molecule can be accomplished as described in Anzalone et al. 2019. Nature. 576:149-157, particularly at pg. 3, FIG. 2a-2b, and Extended Data FIGS. 5a-c.

In an embodiment, the genetic modifying system is a PASTE system, such as one described in e.g., Yarnell et al., Nat. Biotech. 2022. doi.org/10.1038/s41587-022-01527-4.

### CRISPR Associated Transposase (CAST) Systems

In an embodiment, the genetic modifying system is a CRISPR Associated Transposase (“CAST”) system. A CAST system can include a Cas protein that is catalytically inactive, or engineered to be catalytically active (e.g., have nickase or nuclease activity), and further comprises a transposase (or subunits thereof) that catalyze RNA-guided DNA transposition. Such systems are able to insert DNA sequences at a target site in a DNA molecule without relying on host cell repair machinery. CAST systems can be Class1 or Class 2 CAST systems. An example Class 1 system is described in Klompe et al. Nature, doi: 10.1038/s41586-019-1323, which is incorporated herein by reference. An example Class 2 system is described in Strecker et al. Science. 10/1126/science. aax9181 (2019), and PCT/US2019/066835 which are incorporated herein by reference.

### IscB Systems

In an embodiment, the nucleic acid-guided nucleases herein may be IscB proteins. An IscB protein may comprise an X domain and a Y domain as described herein. In some examples, the IscB proteins may form a complex with one or more guide molecules. In some cases, the IscB proteins may form a complex with one or more hRNA molecules which serve as a scaffold molecule and comprise guide sequences. In some examples, the IscB proteins are CRISPR-associated proteins, e.g., the loci of the nucleases are associated with an CRISPR array. In some examples, the IscB proteins are not CRISPR-associated.

In some examples, the IscB protein may be homolog or ortholog of IscB proteins described in Kapitonov V V et al., ISC, a Novel Group of Bacterial and Archaeal DNA Transposons That Encode Cas9 Homologs, J Bacteriol. 2015 Dec. 28; 198 (5): 797-807. doi: 10.1128/JB.00783-15, which is incorporated by reference herein in its entirety.

In an embodiment, the IscBs may comprise one or more domains, e.g., one or more of a X domain (e.g., at N-terminus), a RuvC domain, a Bridge Helix domain, and a Y domain (e.g., at C-terminus). In some examples, the nucleic-acid guided nuclease comprises an N-terminal X domain, a RuvC domain (e.g., including a RuvC-I, RuvC-II, and RuvC-III subdomains), a Bridge Helix domain, and a C-terminal Y domain. In some examples, the nucleic-acid guided nuclease comprises an N-terminal X domain, a RuvC domain (e.g., including a RuvC-I, RuvC-II, and RuvC-III subdomains), a Bridge Helix domain, an HNH domain, and a C-terminal Y domain.

In an embodiment, the nucleic acid-guided nucleases may have a small size. For example, the nucleic acid-guided nucleases may be no more than 50, no more than 100, no more than 150, no more than 200, no more than 250, no more than 300, no more than 350, no more than 400, no more than 450, no more than 500, no more than 550, no more than 600, no more than 650, no more than 700, no more than 750, no more than 800, no more than 850, no more than 900, no more than 950, or no more than 1000 amino acids in length.

In some examples, the IscB protein shares at least 80%, at least 85%, at least 90%, at least 95%, at least 99%, or 100% sequence identity with a IscB protein selected from Table

TABLE 3
3

| No. | Proteins | Sequences |
|---|---|---|
| 1 | IscB(−HNH) | 1 mstdatlirt tpshaeadat dtlvatplmp prrvispwpg pgegqslmri pvvdirgmal |
| | EFH81386 | 61 mpctpakarh llksgnarpk rnklglfyvq lsyeqepdnq slvagvdpgs kfeglsvvgt |
| | | 121 kdtvlnlmve apdhvkgavq trrtmrrarr qrkwrrpkrf hnrlnrmqri ppstrsrwea |
| | | 181 karivahlrt ilpftdvvve dvqavtrkgk ggtwngsfsp vqvgkehlyr llramgltlh |
| | | 241 lregwqtkel reqhglkktk skskqsfesh avdswvlaas isgaehptct rlwymvpail |
| | | 301 hrrqlhrlqa skggvrkpyg gtrslgvkrg tlvehkkygr ctvggvdrkr ntislheyrt |
| | | 361 ntrltqaakv etcrvltwls wrswllrgkr tsskgkgshs s (SEQ ID NO: 10) |
| 2 | IscB(+HNH) | 1 mqpakqqnwv fqingdkqpl dminpgrcre lqnrgklasf rrfpyvviqq qtienpqtke |
| | TAE54104.1 | 61 yilkidpgsq wtgfaiqcgn dilfraelnh rgeaikfdlv krawfrrgrr srnlryrkkr |
| | | 121 lnrakpegwl apsirhrvlt vetwikrfmr ycpiawieie qvrfdtqkla npeidgveyq |
| | | 181 qgelqgyevr eyllqkwgrk cayegtenvp levehiqsks kggssrignl tlachvenvk |
| | | 241 kgnldvrdfl akspdilnqv lenstkplkd aaavnstrya ivkmaksice nvkessgart |
| | | 301 kmnrvrqgle kthsldaacv gesgasirvl tdrpllitck ghgsrqsirv nasgfpavkn |
| | | 361 aktvfthiaa gdvvrftigk drkkaqagty tarvktptpk gfevlidgar islstmsnvv |
| | | 421 fvhrsdgygy el (SEQ ID NO: 11) |
| 3 | IscB(+HNH) | 1 mavfvidkhk rplmpcsekr arlllergra vvhrqvpfvi rlkdrtvqhs avqplrvald |
| | WP_038093640.1 | 61 pgsratgmal vrekntvdtg tgevyreria lnlfelvhrg hrireqldqr rnfrrrrrga |
| | | 121 nlryraprfd nrrrppgwla pslqhrvdtt mawvrrlerw apasaigiet vrfdtqrlqn |
| | | 181 peisgveyqq galagcevre yllekwgrkc aycgaenvpl eiehivpksr ggsdrvsnla |
| | | 241 lacracnqak gnrdvrafla dqperlaril aqakaplkda aavnatrwal yralvdtglp |
| | | 301 veagtggrtk wnrtrlglpk thaldalcvg qvdqvrhwrv pvlgircagr gsyrrtrltr |
| | | 361 hgfprgyltr nksafgfqtg dliravvtkg kkagtylgri airasgsfni qtpmgvvqgi |
| | | 421 hhrfctllqr adgygyfvqp kpteaalssp rlkagvssag n (SEQ ID NO: 12) |
| 4 | IscB(+HNH) | 1 mttnvvfvid tnqkplqpcs aavarklllr gkaamfrryp aviilkkevd svgkpkielr |
| | WP_052490348.1 | 61 idpgskytgf alvdskdnad fiiwgteleh rgaaickelt krsairrsrr nrktryrkkr |
| | | 121 ferrkpegwl apslqhrvdt tltwvkrick fvpimsisve qvkfdlqkle nsdiqgieyq |
| | | 181 qgtlagytlr eallehwgrk caycdvenvf leiehiypks kggsdkfsnl tlachkcnin |
| | | 241 kgnksidefl lsdhkrleqi klhqkktlkd aaavnatrkk lvttlqektf lnvlvsdgas |
| | | 301 tkmtrlsssl akrhwidagc vnttlivilk tlqplqvken ghgnkqfvtm daygfprksy |
| | | 361 epkkvrkdwk agdiirvtkk dgtmlmgrvk kaakklvyip fggkeasfss enakaihrsd |
| | | 421 gyrysfaaid sellqkmat (SEQ ID NO: 13) |
| 5 | IscB(+HNH) | 1 mpnkyafvld skgklldptk skkawylirk gkaslveeyp liiklkrevp kdqvnsdkli |
| | WP_015325818.1 | 61 lgiddgtkkv gfalvqkcqt knkvlfkavm eqrqdvskkm eerrgyrryr rshkryrpar |
| | | 121 fdnrssskrk grippsilqk kqailrvvnk lkkyiridki vledvsidir kltegrelyn |
| | | 181 weyqesnrld enlrkatlyr ddcteqlegt tetmlhahhi mprrdggads iynlitlcka |
| | | 241 chkdkvdnne yqykdqflai idskelsdlk sashvmqgkt wlrdklskia qleitsggnt |
| | | 301 ankridyeie kshsndaict tgllpvdnid dikeyyikpl rkkskakike lkcfrqrdlv |
| | | 361 kytkrngety tgyitslrik nnkynskven fstlkgkifr gygfrnltll nrpkglmiv |
| | | (SEQ ID NO: 14) |
| 6 | sp\|G3ECR1\|CAS9 | 1 mlfnkciiis inldfsnkek cmtkpysigl digtnsvgwa vitdnykvps kkmkvlgnts |
| | STRTR | 61 kkyikknllg vllfdsgita egrrlkrtar rrytrrrnri lylqeifste matlddaffq |
| | | 121 rlddsflvpd dkrdskypif gnlveekvyh defptiyhlr kyladstkka dlrlvylala |
| | | 181 hmikyrghfl iegefnsknn diqknfqdfl dtynaifesd lslenskqle eivkdkiskl |
| | | 241 ekkdrilklf pgeknsgifs eflklivgnq adfrkcfnld ekaslhfske sydedletll |
| | | 301 gyigddysdv flkakklyda illsgfltvt dneteaplss amikrynehk edlallkeyi |
| | | 361 rnislktyne vfkddtkngy agyidgktnq edfyvylknl laefegadyf lekidredfl |
| | | 421 rkqrtfdngs ipyqihlqem raildkqakf ypflaknker iekiltfrip yyvgplargn |
| | | 481 sdfawsirkr nekitpwnfe dvidkessae afinrmtsfd lylpeekvlp khsllyetfn |
| | | 541 vyneltkvrf iaesmrdyqf ldskqkkdiv rlyfkdkrkv tdkdiieylh aiygydgiel |
| | | 601 kgiekqfnss lstyhdllni indkefldds sneaiieeii htltifedre mikqrlskfe |
| | | 661 nifdksvlkk lsrrhytgwg klsaklingi rdeksgntil dyliddgisn rnfmqlihdd |
| | | 721 alsfkkkiqk aqiigdedkg nikevvkslp gspaikkgil qsikivdelv kvmggrkpes |
| | | 781 ivvemarenq ytnqgksnsq qrlkrleksl kelgskilke nipaklskid nnalqndrly |
| | | 841 lyylqngkdm ytgddldidr lsnydidhii pqaflkdnsi dnkvlvssas nrgksddfps |
| | | 901 levvkkrktf wyqllkskli sqrkfdnltk aerggllped kagfiqrqlv etrqitkhva |
| | | 961 rlldekfnnk kdennravrt vkiitlkstl vsqfrkdfel ykvreindfh hahdaylnav |
| | | 1021 iasallkkyp klepefvygd ypkynsfrer ksatekvyfy snimnifkks isladgrvie |
| | | 1081 rplievneet gesvwnkesd latvrrvlsy pqvnvvkkve eqnhgldrgk pkglfnanls |
| | | 1141 skpkpnsnen lvgakeyldp kkyggyagis nsfavlvkgt iekgakkkit nvlefqgisi |
| | | 1201 ldrinyrkdk lnfllekgyk dieliielpk yslfelsdgs rrmlasilst nnkrgeihkg |
| | | 1261 nqiflsqkfv kllyhakris ntinenhrky venhkkefee lfyyilefne nyvgakkngk |
| | | 1321 llnsafqswq nhsidelcss figptgserk glfeltsrgs aadfeflgvk ipryrdytps |
| | | 1381 slikdatlih qsvtglyetr idlaklgeg (SEQ ID NO: 15) |
| 7 | sp\|J7RUA5\|CAS9 | 1 mkrnyilgld igitsvgygi idyetrdvid agvrlfkean vennegrrsk rgarrlkrrr |
| | STAAU | 61 rhriqrvkkl lfdynlltdh selsginpye arvkglsqkl seeefsaall hlakrrgvhn |
| | | 121 vneveedtgn elstkeqisr nskaleekyv aelqlerlkk dgevrgsinr fktsdyvkea |
| | | 181 kqllkvqkay hqldqsfidt yidlletrrt yyegpgegsp fgwkdikewy emlmghctyf |
| | | 241 peelrsvkya ynadlynaln dlnnlvitrd enekleyyek fqiienvfkq kkkptlkqia |
| | | 301 keilvneedi kgyrvtstgk peftnlkvyh dikditarke iienaelldq iakiltiyqs |
| | | 361 sediqeeltn lnseltqeei eqisnlkgyt gthnlslkai nlildelwht ndnqiaifnr |
| | | 421 lklvpkkvdl sqqkeipttl vddfilspvv krsfiqsikv inaiikkygl pndiiielar |
| | | 481 eknskdaqkm inemqkrnrq tnerieeiir ttgkenakyl iekiklhdmq egkclyslea |
| | | 541 ipledllnnp fnyevdhiip rsvsfdnsfn nkvlvkqeen skkgnrtpfq ylsssdskis |
| | | 601 yetfkkhiln lakgkgrisk tkkeylleer dinrfsvqkd finrnlvdtr yatrglmnll |
| | | 661 rsyfrvnnld vkvksinggf tsflrrkwkf kkernkgykh haedaliian adfifkewkk |
| | | 721 ldkakkvmen qmfeekqaes mpeieteqey keifitphqi khikdfkdyk yshrvdkkpn |
| | | 781 relindtlys trkddkgntl ivnninglyd kdndklkkli nkspekllmy hhdpqtyqkl |
| | | 841 klimeqygde knplykyyee tgnyltkysk kdngpvikki kyygnklnah lditddypns |
| | | 901 rnkvvklslk pyrfdvyldn gvykfvtvkn ldvikkenyy evnskcyeea kklkkisnqa |
| | | 961 efiasfynnd likingelyr vigvnndlln rievnmidit yreylenmnd krppriikti |
| | | 1021 asktqsikky stdilgnlye vkskkhpqii kkg (SEQ ID NO: 16) |
| 8 | Streptococcus_ | 1 kysigldigt nsvgwavitd eykvpskkfk vlgntdrhsi kknligallf dsgetaeatr |
| | pyogenes_SF370 | 61 lkrtarrryt rrknricylq eifsnemakv ddsffhrlee sflveedkkh erhpifgniv |
| | | 121 devayhekyp tiyhlrkklv dstdkadlrl iylalahmik frghfliegd lnpdnsdvdk |
| | | 181 lfiqlvqtyn qlfeenpina sgvdakails arlsksrrle nliaqlpgek knglfgnlia |
| | | 241 lslgltpnfk snfdlaedak lqlskdtydd dldnllaqig dqyadlflaa knlsdaills |
| | | 301 dilrvnteit kaplsasmik rydehhqdlt llkalvrqql pekykeiffd qskngyagyi |
| | | 361 dggasqeefy kfikpilekm dgteellvkl nredllrkqr tfdngsiphq ihlgelhail |
| | | 421 rrqedfypfl kdnrekieki ltfripyyvg plargnsrfa wmtrkseeti tpwnfeevvd |
| | | 481 kgasaqsfie rmtnfdknlp nekvlpkhsl lyeyftvyne ltkvkyvteg mrkpaflsge |
| | | 541 qkkaivdllf ktnrkvtvkq lkedyfkkie cfdsveisgv edrfnaslgt yhdllkiikd |
| | | 601 kdfldneene diledivltl tlfedremie erlktyahlf ddkvmkqlkr rrytgwgrls |
| | | 661 rklingirdk qsgktildfl ksdgfanrnf mqlihddslt fkediqkaqv sgqgdslheh |
| | | 721 ianlagspai kkgilqtvkv vdelvkvmgr hkpeniviem arenqttqkg qknsrermkr |
| | | 781 ieegikelgs qilkehpven tqlqneklyl yylqngrdmy vdqeldinrl sdydvdhivp |
| | | 841 qsflkddsid nkvltrsdkn rgksdnvpse evvkkmknyw rqllnaklit qrkfdnltka |
| | | 901 ergglseldk agfikrqlve trqitkhvaq ildsrmntky dendklirev kvitlksklv |
| | | 961 sdfrkdfqfy kvreinnyhh ahdaylnavv gtalikkypk lesefvygdy kvydvrkmia |
| | | 1021 kseqeigkat akyffysnim nffkteitla ngeirkrpli etngetgeiv wdkgrdfatv |
| | | 1081 rkvlsmpqvn ivkktevqtg gfskesilpk rnsdkliark kdwdpkkygg fdsptvaysv |
| | | 1141 lvvakvekgk skklksvkel lgitimerss feknpidfle akgykevkkd liiklpkysl |
| | | 1201 felengrkrm lasagelqkg nelalpskyv nflylashye klkgspedne qkqlfveqhk |
| | | 1261 hyldeiieqi sefskrvila danldkvlsa ynkhrdkpir eqaeniihlf tltnlgapaa |
| | | 1321 fkyfdttidr krytstkevl datlihqsit glyetridls qlggd (SEQ ID NO: 17) |
| [symbol] o. | Proteins | Domains and amino acid positions |
| | IscB(−HNH) | X domain: 51-97 |
| | EFH81386 | RuvC-I: 104-118 |
| | | Bridge Helix: 140-160 |
| | | RuvC-II: 169-212 |
| | | RuvC-III: 226-278 |
| | IscB(+HNH) | X domain: 11-56 |
| | TAE54104.1 | RuvC-I: 63-77 |
| | | Bridge Helix: 100-121 |
| | | RuvC-II: 129-172 |
| | | HNH: 211-243 |
| | | RuvC-III: 279-321 |
| | IscB(+HNH) | X domain: 4-50 |
| | WP_038093640.1 | RuvC-I: 57-71 |
| | | Bridge Helix: 108-129 |
| | | RuvC-II: 138-181 |
| | | HNH: 220-252 |
| | IscB(+HNH) | X domain: 7-52 |
| | WP_052490348.1 | RuvC-I: 59-73 |
| | | Bridge Helix: 100-121 |
| | | RuvC-II: 129-172 |
| | | HNH: 211-243 |
| | | RuvC-III: 279-322 |
| | IscB(+HNH) | X domain: 7-52 |
| | WP_015325818.1 | RuvC-I: 61-75 |
| | | Bridge Helix: 101-121 |
| | | RuvC-II: 132-175 |
| | | HNH: 215-247 |
| | | RuvC-III: 284-327 |
| | sp\|G3ECR1\| | RuvC-I: 28-42 |
| | CAS9_STRTR | Bridge Helix: 85-108 |
| | | Rec: 118-736 |
| | | RuvC-II: 750-799 |
| | | HNH: 864-896 |
| | | RuvC-III: 957-1019 |
| | | PAM Interaction (PI): 1119-1409 |
| | sp\|J7RUA5\| | RuvC-I: 7-21 |
| | CAS9_STAAU | Bridge Helix: 49-72 |
| | | Rec: 80-433 |
| | | RuvC-II: 445-493 |
| | | HNH: 553-585 |
| | | RuvC-III: 654-709 |
| | | PAM Interaction (PI): 789-1053 |
| | Streptococcus_ | RuvC-I: 4-18 |
| | pyogenes_ | Bridge Helix: 61-84 |
| | SF370 | Rec: 94-718 |
| | | RuvC-II: 725-774 |
| | | HNH: 833-865 |
| | | RuvC-III: 926-988 |
| | | PAM Interaction (PI): 1099-1365 |

[symbol]

indicates data missing or illegible when filed

### X Domains

In an embodiment, the IscB proteins comprise an X domain, e.g., at its N-terminal.

In certain embodiments, the X domain include the X domains in Table 3. Examples of the X domains also include any polypeptides a structural similarity and/or sequence similarity to a X domain described in the art. In some examples, the X domain may have an amino acid sequence that share at least 50%, at least 55%, at least 60%, at least 5%, at least 70%, at least 75%, at least 80%, at least 85%, at least 90%, at least 95%, at least 99%, or 100% sequence identity with X domains in Table 3.

In some examples, the X domain may be no more than 10, no more than 20, no more than 30, no more than 40, no more than 50, no more than 60, no more than 70, no more than 80, no more than 90, or no more than 100 amino acids in length. For example, the X domain may be no more than 50 amino acids in length, such as comprising 2 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acids in length.

### Y Domain

In an embodiment, the IscB proteins comprise a Y domain, e.g., at its C-terminal.

In certain embodiments, the X domain include Y domains in Table 3. Examples of the Y domain also include any polypeptides a structural similarity and/or sequence similarity to a Y domain described in the art. In some examples, the Y domain may have an amino acid sequence that share at least 50%, at least 55%, at least 60%, at least 5%, at least 70%, at least 75%, at least 80%, at least 85%, at least 90%, at least 95%, at least 99%, or 100% sequence identity with Y domains in Table 3.

### RuvC Domain

In an embodiment, the IscB proteins comprises at least one nuclease domain. In certain embodiments, the IscB proteins comprise at least two nuclease domains. In certain embodiments, the one or more nuclease domains are only active upon presence of a cofactor. In certain embodiments, the cofactor is Magnesium (Mg). In embodiments where more than one nuclease domain is present and the substrate is a double-strand polynucleotide, the nuclease domains each cleave a different strand of the double-strand polynucleotide. In certain embodiments, the nuclease domain is a RuvC domain.

The IscB proteins may comprise a RuvC domain. The RuvC domain may comprise multiple subdomains, e.g., RuvC-I, RuvC-II and RuvC-III. The subdomains may be separated by interval sequences on the amino acid sequence of the protein.

In certain embodiments, examples of the RuvC domain include those in Table 3. Examples of the RuvC domain also include any polypeptides a structural similarity and/or sequence similarity to a RuvC domain described in the art. For example, the RuvC domain may share a structural similarity and/or sequence similarity to a RuvC of Cas9. In some examples, the RuvC domain may have an amino acid sequence that share at least 50%, at least 55%, at least 60%, at least 5%, at least 70%, at least 75%, at least 80%, at least 85%, at least 90%, at least 95%, at least 99%, or 100% sequence identity with RuvC domains in Table 3.

### Bridge Helix

The IscB proteins comprise a bridge helix (BH) domain. The bridge helix domain refers to a helix and arginine rich polypeptide. The bridge helix domain may be located next to anyone of the amino acid domains in the nucleic-acid guided nuclease. In an embodiment, the bridge helix domain is next to a RuvC domain, e.g., next to RuvC-I, RuvC-II, or RuvC-III subdomain. In one example, the bridge helix domain is between a RuvC-1 and RuvC2 subdomains.

The bridge helix domain may be from 10 to 100, from 20 to 60, from 30 to 50, e.g., 35, 36, 37, 38, 39, 40, 41, 42, 43, 44, 45, 46 or 47, 48, 49, or 50 amino acids in length. Examples of bridge helix includes the polypeptide of amino acids 60-93 of the sequence of S. pyogenes Cas9.

In certain embodiments, examples of the BH domain include those in Table 3. Examples of the BH domain also include any polypeptides a structural similarity and/or sequence similarity to a BH domain described in the art. For example, the BH domain may share a structural similarity and/or sequence similarity to a BH domain of Cas9. In some examples, the BH domain may have an amino acid sequence that share at least 50%, at least 55%, at least 60%, at least 5%, at least 70%, at least 75%, at least 80%, at least 85%, at least 90%, at least 95%, at least 99%, or 100% sequence identity with BH domains in Table 3.

### HNH Domain

The IscB proteins comprise an HNH domain. In certain embodiments, at least one nuclease domain shares a substantial structural similarity or sequence similarity to a HNH domain described in the art.

In some examples, the nucleic acid-guided nuclease comprises a HNH domain and a RuvC domain. In the cases where the RuvC domain comprises RuvC-I, RuvC-II, and RuvC-III domain, the HNH domain may be located between the Ruv C II and RuvC III subdomains of the RuvC domain.

In certain embodiments, examples of the HNH domain include those in Table 3. Examples of the HNH domain also include any polypeptides a structural similarity and/or sequence similarity to a HNH domain described in the art. For example, the HNH domain may share a structural similarity and/or sequence similarity to a HNH domain of Cas9. In some examples, the HNH domain may have an amino acid sequence that share at least 50%, at least 55%, at least 60%, at least 5%, at least 70%, at least 75%, at least 80%, at least 85%, at least 90%, at least 95%, at least 99%, or 100% sequence identity with HNH domains in Table 3.

hRNA

In some examples, the IscB proteins capable of forming a complex with one or more hRNA molecules. The hRNA complex can comprise a guide sequence and a scaffold that interacts with the IscB polypeptide. An hRNA molecules may form a complex with an IscB polypeptide nuclease or IscB polypeptide, and direct the complex to bind with a target sequence. In certain example embodiments, the hRNA molecule is a single molecule comprising a scaffold sequence and a spacer sequence. In certain example embodiments, the spacer is 5′ of the scaffold sequence. In certain example embodiments, the hRNA molecule may further comprise a conserved nucleic acid sequence between the scaffold and spacer portions.

As used herein, a heterologous hRNA molecule is an hRNA molecule that is not derived from the same species as the IscB polypeptide nuclease, or comprises a portion of the molecule, e.g. spacer, that is not derived from the same species as the IscB polypeptide nuclease, e.g. IscB protein. For example, a heterologous hRNA molecule of a IscB polypeptide nuclease derived from species A comprises a polynucleotide derived from a species different from species A, or an artificial polynucleotide.

### TALE Nucleases

In an embodiment, a TALE nuclease or TALE nuclease system can be used to modify a polynucleotide. In an embodiment, the methods provided herein use isolated, non-naturally occurring, recombinant or engineered DNA binding proteins that comprise TALE monomers or TALE monomers or half monomers as a part of their organizational structure that enable the targeting of nucleic acid sequences with improved efficiency and expanded specificity.

Naturally occurring TALEs or “wild type TALEs” are nucleic acid binding proteins secreted by numerous species of proteobacteria. TALE polypeptides contain a nucleic acid binding domain composed of tandem repeats of highly conserved monomer polypeptides that are predominantly 33, 34 or 35 amino acids in length and that differ from each other mainly in amino acid positions 12 and 13. In advantageous embodiments the nucleic acid is DNA. As used herein, the term “polypeptide monomers”, “TALE monomers” or “monomers” will be used to refer to the highly conserved repetitive polypeptide sequences within the TALE nucleic acid binding domain and the term “repeat variable di-residues” or “RVD” will be used to refer to the highly variable amino acids at positions 12 and 13 of the polypeptide monomers. As provided throughout the disclosure, the amino acid residues of the RVD are depicted using the IUPAC single letter code for amino acids. A general representation of a TALE monomer which is comprised within the DNA binding domain is X₁₋₁₁-(X₁₂X₁₃)-X₁₄₋₃₃ or ₃₄ or ₃₅, where the subscript indicates the amino acid position and X represents any amino acid. X₁₂X₁₃ indicate the RVDs. In some polypeptide monomers, the variable amino acid at position 13 is missing or absent and in such monomers, the RVD consists of a single amino acid. In such cases the RVD may be alternatively represented as X*, where X represents X₁₂ and (*) indicates that X₁₃ is absent. The DNA binding domain comprises several repeats of TALE monomers and this may be represented as (X₁₋₁₁-(X₁₂X₁₃)-X₁₄₋₃₃ or ₃₄ or ₃₅)_{z}, where in an advantageous embodiment, z is at least 5 to 40. In a further advantageous embodiment, z is at least 10 to 26.

The TALE monomers can have a nucleotide binding affinity that is determined by the identity of the amino acids in its RVD. For example, polypeptide monomers with an RVD of N1 can preferentially bind to adenine (A), monomers with an RVD of NG can preferentially bind to thymine (T), monomers with an RVD of HD can preferentially bind to cytosine (C) and monomers with an RVD of NN can preferentially bind to both adenine (A) and guanine (G). In an embodiment, monomers with an RVD of IG can preferentially bind to T. Thus, the number and order of the polypeptide monomer repeats in the nucleic acid binding domain of a TALE determines its nucleic acid target specificity. In an embodiment, monomers with an RVD of NS can recognize all four base pairs and can bind to A, T, G or C. The structure and function of TALEs is further described in, for example, Moscou et al., Science 326:1501 (2009); Boch et al., Science 326:1509-1512 (2009); and Zhang et al., Nature Biotechnology 29:149-153 (2011).

The polypeptides used in methods of the invention can be isolated, non-naturally occurring, recombinant or engineered nucleic acid-binding proteins that have nucleic acid or DNA binding regions containing polypeptide monomer repeats that are designed to target specific nucleic acid sequences.

As described herein, polypeptide monomers having an RVD of HN or NH preferentially bind to guanine and thereby allow the generation of TALE polypeptides with high binding specificity for guanine containing target nucleic acid sequences. In an embodiment, polypeptide monomers having RVDs RN, NN, NK, SN, NH, KN, HN, NQ, HH, RG, KH, RH and SS can preferentially bind to guanine. In an embodiment, polypeptide monomers having RVDs RN, NK, NQ, HH, KH, RH, SS and SN can preferentially bind to guanine and can thus allow the generation of TALE polypeptides with high binding specificity for guanine containing target nucleic acid sequences. In an embodiment, polypeptide monomers having RVDs HH, KH, NH, NK, NQ, RH, RN and SS can preferentially bind to guanine and thereby allow the generation of TALE polypeptides with high binding specificity for guanine containing target nucleic acid sequences. In an embodiment, the RVDs that have high binding specificity for guanine are RN, NH RH and KH. Furthermore, polypeptide monomers having an RVD of NV can preferentially bind to adenine and guanine. In an embodiment, monomers having RVDs of H*, HA, KA, N*, NA, NC, NS, RA, and S* bind to adenine, guanine, cytosine and thymine with comparable affinity.

The predetermined N-terminal to C-terminal order of the one or more polypeptide monomers of the nucleic acid or DNA binding domain determines the corresponding predetermined target nucleic acid sequence to which the polypeptides of the invention will bind. As used herein the monomers and at least one or more half monomers are “specifically ordered to target” the genomic locus or gene of interest. In plant genomes, the natural TALE-binding sites always begin with a thymine (T), which may be specified by a cryptic signal within the non-repetitive N-terminus of the TALE polypeptide; in some cases, this region may be referred to as repeat 0. In animal genomes, TALE binding sites do not necessarily have to begin with a thymine (T) and polypeptides of the invention may target DNA sequences that begin with T, A, G or C. The tandem repeat of TALE monomers always ends with a half-length repeat or a stretch of sequence that may share identity with only the first 20 amino acids of a repetitive full-length TALE monomer and this half repeat may be referred to as a half-monomer. Therefore, it follows that the length of the nucleic acid or DNA being targeted is equal to the number of full monomers plus two.

As described in Zhang et al., Nature Biotechnology 29:149-153 (2011), TALE polypeptide binding efficiency may be increased by including amino acid sequences from the “capping regions” that are directly N-terminal or C-terminal of the DNA binding region of naturally occurring TALEs into the engineered TALEs at positions N-terminal or C-terminal of the engineered TALE DNA binding region. Thus, in certain embodiments, the TALE polypeptides described herein further comprise an N-terminal capping region and/or a C-terminal capping region.

An exemplary amino acid sequence of a N-terminal capping region is:

(SEQ ID NO: 18)
M D P I R S R T P S P A R E L L S G
P Q P D G V Q P T A D R G V S P P A
G G P L D G L P A R R T M S R T R L
P S P P A P S P A F S A D S F S D L
L R Q F D P S L F N T S L F D S L P
P F G A H H T E A A T G E W D E V Q
S G L R A A D A P P P T M R V A V T
A A R P P R A K P A P R R R A A Q P
S D A S P A A Q V D L R T L G Y S Q
Q Q Q E K I K P K V R S T V A Q H H
E A L V G H G F T H A H I V A L S Q
H P A A L G T V A V K Y Q D M I A A
L P E A T H E A I V G V G K Q W S G
A R A L E A L L T V A G E L R G P P
L Q L D T G Q L L K I A K R G G V T
A V E A V H A W R N A L T G A P L N

An exemplary amino acid sequence of a C-terminal capping region is:

(SEQ ID NO: 19)
R P A L E S I V A Q L S R P D P A L
A A L T N D H L V A L A C L G G R P
A L D A V K K G L P H A P A L I K R
T N R R I P E R T S H R V A D H A Q
V V R V L G F F Q C H S H P A Q A F
D D A M T Q F G M S R H G L L Q L F
R R V G V T E L E A R S G T L P P A
S Q R W D R I L Q A S G M K R A K P
S P T S T Q T P D Q A S L H A F A D
S L E R D L D A P S P M H E G D Q T
R A S

As used herein the predetermined “N-terminus” to “C terminus” orientation of the N-terminal capping region, the DNA binding domain comprising the repeat TALE monomers and the C-terminal capping region provide structural basis for the organization of different domains in the d-TALEs or polypeptides of the invention.

The entire N-terminal and/or C-terminal capping regions are not necessary to enhance the binding activity of the DNA binding region. Therefore, in certain embodiments, fragments of the N-terminal and/or C-terminal capping regions are included in the TALE polypeptides described herein.

In certain embodiments, the TALE polypeptides described herein contain a N-terminal capping region fragment that included at least 10, 20, 30, 40, 50, 54, 60, 70, 80, 87, 90, 94, 100, 102, 110, 117, 120, 130, 140, 147, 150, 160, 170, 180, 190, 200, 210, 220, 230, 240, 250, 260 or 270 amino acids of an N-terminal capping region. In certain embodiments, the N-terminal capping region fragment amino acids are of the C-terminus (the DNA-binding region proximal end) of an N-terminal capping region. As described in Zhang et al., Nature Biotechnology 29:149-153 (2011), N-terminal capping region fragments that include the C-terminal 240 amino acids enhance binding activity equal to the full length capping region, while fragments that include the C-terminal 147 amino acids retain greater than 80% of the efficacy of the full length capping region, and fragments that include the C-terminal 117 amino acids retain greater than 50% of the activity of the full-length capping region.

In an embodiment, the TALE polypeptides described herein contain a C-terminal capping region fragment that included at least 6, 10, 20, 30, 37, 40, 50, 60, 68, 70, 80, 90, 100, 110, 120, 127, 130, 140, 150, 155, 160, 170, 180 amino acids of a C-terminal capping region. In certain embodiments, the C-terminal capping region fragment amino acids are of the N-terminus (the DNA-binding region proximal end) of a C-terminal capping region. As described in Zhang et al., Nature Biotechnology 29:149-153 (2011), C-terminal capping region fragments that include the C-terminal 68 amino acids enhance binding activity equal to the full-length capping region, while fragments that include the C-terminal 20 amino acids retain greater than 50% of the efficacy of the full-length capping region.

In certain embodiments, the capping regions of the TALE polypeptides described herein do not need to have identical sequences to the capping region sequences provided herein. Thus, In an embodiment, the capping region of the TALE polypeptides described herein have sequences that are at least 50%, 60%, 70%, 80%, 85%, 90%, 91%, 92%, 93%, 94%, 95%, 96%, 97%, 98% or 99% identical or share identity to the capping region amino acid sequences provided herein. Sequence identity is related to sequence homology. Homology comparisons may be conducted by eye, or more usually, with the aid of readily available sequence comparison programs. These commercially available computer programs may calculate percent (%) homology between two or more sequences and may also calculate the sequence identity shared by two or more amino acid or nucleic acid sequences. In some preferred embodiments, the capping region of the TALE polypeptides described herein have sequences that are at least 95% identical or share identity to the capping region amino acid sequences provided herein.

Sequence homologies can be generated by any of a number of computer programs known in the art, which include, but are not limited to, BLAST or FASTA. Suitable computer programs for carrying out alignments like the GCG Wisconsin Bestfit package may also be used. Once the software has produced an optimal alignment, it is possible to calculate % homology, preferably % sequence identity. The software typically does this as part of the sequence comparison and generates a numerical result.

In an embodiment described herein, the TALE polypeptides of the invention include a nucleic acid binding domain linked to the one or more effector domains. The terms “effector domain” or “regulatory and functional domain” refer to a polypeptide sequence that has an activity other than binding to the nucleic acid sequence recognized by the nucleic acid binding domain. By combining a nucleic acid binding domain with one or more effector domains, the polypeptides of the invention may be used to target the one or more functions or activities mediated by the effector domain to a particular target DNA sequence to which the nucleic acid binding domain specifically binds.

In an embodiment of the TALE polypeptides described herein, the activity mediated by the effector domain is a biological activity. For example, In an embodiment the effector domain is a transcriptional inhibitor (i.e., a repressor domain), such as an mSin interaction domain (SID). SID4X domain or a Krüppel-associated box (KRAB) or fragments of the KRAB domain. In an embodiment, the effector domain is an enhancer of transcription (i.e., an activation domain), such as the VP16, VP64 or p65 activation domain. In an embodiment, the nucleic acid binding is linked, for example, with an effector domain that includes but is not limited to a transposase, integrase, recombinase, resolvase, invertase, protease, DNA methyltransferase, DNA demethylase, histone acetylase, histone deacetylase, nuclease, transcriptional repressor, transcriptional activator, transcription factor recruiting, protein nuclear-localization signal or cellular uptake signal.

In an embodiment, the effector domain is a protein domain which exhibits activities which include but are not limited to transposase activity, integrase activity, recombinase activity, resolvase activity, invertase activity, protease activity, DNA methyltransferase activity, DNA demethylase activity, histone acetylase activity, histone deacetylase activity, nuclease activity, nuclear-localization signaling activity, transcriptional repressor activity, transcriptional activator activity, transcription factor recruiting activity, or cellular uptake signaling activity. Other preferred embodiments of the invention may include any combination of the activities described herein.

Other preferred tools for genome editing for use in the context of this invention include zinc finger systems and TALE systems. One type of programmable DNA-binding domain is provided by artificial zinc-finger (ZF) technology, which involves arrays of ZF modules to target new DNA-binding sites in the genome. Each finger module in a ZF array targets three DNA bases. A customized array of individual zinc finger domains is assembled into a ZF protein (ZFP).

### Zinc Finger Nucleases

Zinc Finger proteins can comprise a functional domain. The first synthetic zinc finger nucleases (ZFNs) were developed by fusing a ZF protein to the catalytic domain of the Type IIS restriction enzyme FokI. (Kim, Y. G. et al., 1994, Chimeric restriction endonuclease, Proc. Natl. Acad. Sci. U.S.A. 91, 883-887; Kim, Y. G. et al., 1996, Hybrid restriction enzymes: zinc finger fusions to Fok I cleavage domain. Proc. Natl. Acad. Sci. U.S.A. 93, 1156-1160). Increased cleavage specificity can be attained with decreased off target activity by use of paired ZFN heterodimers, each targeting different nucleotide sequences separated by a short spacer. (Doyon, Y. et al., 2011, Enhancing zinc-finger-nuclease activity with improved obligate heterodimeric architectures. Nat. Methods 8, 74-79). ZFPs can also be designed as transcription activators and repressors and have been used to target many genes in a wide variety of organisms. Exemplary methods of genome editing using ZFNs can be found for example in U.S. Pat. Nos. 6,534,261, 6,607,882, 6,746,838, 6,794,136, 6,824,978, 6,866,997, 6,933,113, 6,979,539, 7,013,219, 7,030,215, 7,220,719, 7,241,573, 7,241,574, 7,585,849, 7,595,376, 6,903,185, and 6,479,626, all of which are specifically incorporated by reference.

### Meganucleases

In an embodiment, a meganuclease or system thereof can be used to modify a polynucleotide. Meganucleases, which are endodeoxyribonucleases characterized by a large recognition site (double-stranded DNA sequences of 12 to 40 base pairs). Exemplary methods for using meganucleases can be found in U.S. Pat. Nos. 8,163,514, 8,133,697, 8,021,867, 8,119,361, 8,119,381, 8,124,369, and 8,129,134, which are specifically incorporated herein by reference.

### RNAi

In certain embodiments, the genetic modifying agent is RNAi (e.g., shRNA). As used herein, “gene silencing” or “gene silenced” in reference to an activity of an RNAi molecule, for example a siRNA or miRNA refers to a decrease in the mRNA level in a cell for a target gene by at least about 5%, about 10%, about 20%, about 30%, about 40%, about 50%, about 60%, about 70%, about 80%, about 90%, about 95%, about 99%, about 100% of the mRNA level found in the cell without the presence of the miRNA or RNA interference molecule. In one preferred embodiment, the mRNA levels are decreased by at least about 70%, about 80%, about 90%, about 95%, about 99%, about 100%.

As used herein, the term “RNAi” refers to any type of interfering RNA, including but not limited to, siRNAi, shRNAi, endogenous microRNA and artificial microRNA. For instance, it includes sequences previously identified as siRNA, regardless of the mechanism of down-stream processing of the RNA (i.e. although siRNAs are believed to have a specific method of in vivo processing resulting in the cleavage of mRNA, such sequences can be incorporated into the vectors in the context of the flanking sequences described herein). The term “RNAi” can include both gene silencing RNAi molecules, and also RNAi effector molecules which activate the expression of a gene.

As used herein, a “siRNA” refers to a nucleic acid that forms a double stranded RNA, which double stranded RNA has the ability to reduce or inhibit expression of a gene or target gene when the siRNA is present or expressed in the same cell as the target gene. The double stranded RNA siRNA can be formed by the complementary strands. In one embodiment, a siRNA refers to a nucleic acid that can form a double stranded siRNA. The sequence of the siRNA can correspond to the full-length target gene, or a subsequence thereof. Typically, the siRNA is at least about 15-50 nucleotides in length (e.g., each complementary sequence of the double stranded siRNA is about 15-50 nucleotides in length, and the double stranded siRNA is about 15-50 base pairs in length, preferably about 19-30 base nucleotides, preferably about 20-25 nucleotides in length, e.g., 20, 21, 22, 23, 24, 25, 26, 27, 28, 29, or 30 nucleotides in length).

As used herein “shRNA” or “small hairpin RNA” (also called stem loop) is a type of siRNA. In one embodiment, these shRNAs are composed of a short, e.g. about 19 to about 25 nucleotide, antisense strand, followed by a nucleotide loop of about 5 to about 9 nucleotides, and the analogous sense strand. Alternatively, the sense strand can precede the nucleotide loop structure and the antisense strand can follow.

The terms “microRNA” or “miRNA” are used interchangeably herein are endogenous RNAs, some of which are known to regulate the expression of protein-coding genes at the posttranscriptional level. Endogenous microRNAs are small RNAs naturally present in the genome that are capable of modulating the productive utilization of mRNA. The term artificial microRNA includes any type of RNA sequence, other than endogenous microRNA, which is capable of modulating the productive utilization of mRNA. MicroRNA sequences have been described in publications such as Lim, et al., Genes & Development, 17, p. 991-1008 (2003), Lim et al Science 299, 1540 (2003), Lee and Ambros Science, 294, 862 (2001), Lau et al., Science 294, 858-861 (2001), Lagos-Quintana et al, Current Biology, 12, 735-739 (2002), Lagos Quintana et al, Science 294, 853-857 (2001), and Lagos-Quintana et al, RNA, 9, 175-179 (2003), which are incorporated herein by reference. Multiple microRNAs can also be incorporated into a precursor molecule. Furthermore, miRNA-like stem-loops can be expressed in cells as a vehicle to deliver artificial miRNAs and short interfering RNAs (siRNAs) for the purpose of modulating the expression of endogenous genes through the miRNA and or RNAi pathways.

As used herein, “double stranded RNA” or “dsRNA” refers to RNA molecules that are comprised of two strands. Double-stranded molecules include those comprised of a single RNA molecule that doubles back on itself to form a two-stranded structure. For example, the stem loop structure of the progenitor molecules from which the single-stranded miRNA is derived, called the pre-miRNA (Bartel et al. 2004. Cell 1 16:281-297), comprises a dsRNA molecule.

### Example Engineered Reporter Polynucleotides

As previously described, one or more CREs of the present invention can be operably linked to a reporter polynucleotide so as to allow for cell type, cell state, tissue type, and/or environmental specific CRE-based reporter assays. CRE-Based reporter assays are generally known in the art and the CREs of the present invention can be used in place of conventional CREs in such assays. Described in certain example embodiments, herein are engineered reporter polynucleotides comprising one or more CREs of the present invention, and one or more reporter polynucleotides, wherein the one or more reporter polynucleotides is/are operatively coupled to the one or more of CREs. In an embodiment, one or more of the one or more CREs are identified CREs, engineered CREs, or both.

In an embodiment, expression of the reporter polynucleotide produces a detectable signal. In an embodiment, loss of expression of the reporter is measured as the detectable signal indicative of a desired specific cell type, cell state, tissue type, or environment. This configuration can be employed when one or more CREs is/are a silencer or insulator.

In an embodiment, the reporter polynucleotide encodes a reporter gene product; comprises or encodes a genetic modification system or component thereof; comprises a transcribable barcode; comprises a DNA barcode; comprises a target sequence for a sequence-specific binding molecule or system; comprises a DNA origami reporter system or a component thereof; comprises or encodes an RNAi molecule; comprises or encodes an aptamer; or any combination thereof.

In an embodiment, the reporter gene product is an optically active protein, enzymatic protein, or other protein that can produce a detectable signal when expressed. Examples of such proteins are described elsewhere herein in context with selectable markers and tags in association with the vectors elsewhere herein. In an embodiment the reporter gene product is an antibody, affibody, nanobody, antigen binding fragment, etc. Such molecules are described in greater detail elsewhere herein.

In an embodiment, the reporter polynucleotide comprises or encodes a target sequence for a sequence-specific binding molecule or system. Exemplary sequence-specific binding molecules and/or systems include, without limitation, aptamers, antibodies, RNAi molecules, RNA guided nuclease systems (e.g., CRISPR-Cas, IscB, and OMEGA systems), ZFNs, and/or the like. Such molecules and systems are described in greater detail elsewhere herein. The systems can be configured to detect the target in the reporter polynucleotide, by any conventional system, method, or device, including but not limited to those described herein.

In an embodiment, when a reporter target molecule, such as for a CRISRP-Cas system is expressed in a specific cell in which the CREs of the present invention are expressed, the reporter target molecule can be detected using Cas-13 or Cas12 collateral activity based assay and/or device (See e.g., Mustafa and Makhawi et al., Biotechnology. 2021. 59(3); and Petri and Pattanayak. CRISPR J. 2018. 1 (3): 209, doi.org/10.1089/crispr.2018.29018.kpe). The reporter target sequence can be isolated from the cell in which it is expressed prior to detection. In an embodiment, the target reporter sequence is not isolated from a cell prior to a detection method. Cas13s non-specific RNase activity can be leveraged to cleave reporters upon target recognition, allowing for the design of sensitive and specific diagnostics using Cas13, including single nucleotide variants, detection based on rRNA sequences, screening for drug resistance, monitoring microbe outbreaks, genetic perturbations, and screening of environmental samples, as described, for example, in PCT/US18/054472 filed Oct. 22, 2018 at [0183]-[0327], incorporated herein by reference. Reference is made to WO 2017/219027, WO2018/107129, US20180298445, US 2018-0274017, US 2018-0305773, WO 2018/170340, U.S. application Ser. No. 15/922,837, filed Mar. 15, 2018 entitled “Devices for CRISPR Effector System Based Diagnostics”, PCT/US18/50091, filed Sep. 7, 2018 “Multi-Effector CRISPR Based Diagnostic Systems”, PCT/US18/66940 filed Dec. 20, 2018 entitled “CRISPR Effector System Based Multiplex Diagnostics”, PCT/US18/054472 filed Oct. 4, 2018 entitled “CRISPR Effector System Based Diagnostic”, U.S. Provisional 62/740,728 filed Oct. 3, 2018 entitled “CRISPR Effector System Based Diagnostics for Hemorrhagic Fever Detection”, U.S. Provisional 62/690,278 filed Jun. 26, 2018 and U.S. Provisional 62/767,059 filed Nov. 14, 2018 both entitled “CRISPR Double Nickase Based Amplification, Compositions, Systems and Methods”, U.S. Provisional 62/690,160 filed Jun. 26, 2018 and U.S. Pat. No. 62,767,077 filed Nov. 14, 2018, both entitled “CRISPR/CAS and Transposase Based Amplification Compositions, Systems, And Methods”, U.S. Provisional 62/690,257 filed Jun. 26, 2018 and 62/767,052 filed Nov. 14, 2018 both entitled “CRISPR Effector System Based Amplification Methods, Systems, And Diagnostics”, U.S. Provisional 62/767,076 filed Nov. 14, 2018 entitled “Multiplexing Highly Evolving Viral Variants With SHERLOCK” and 62/767,070 filed Nov. 14, 2018 entitled “Droplet SHERLOCK.” Reference is further made to WO2017/127807, WO2017/184786, WO 2017/184768, WO 2017/189308, WO 2018/035388, WO 2018/170333, WO 2018/191388, WO 2018/213708, WO 2019/005866, PCT/US18/67328 filed Dec. 21, 2018 entitled “Novel CRISPR Enzymes and Systems”, PCT/US18/67225 filed Dec. 21, 2018 entitled “Novel CRISPR Enzymes and Systems” and PCT/US18/67307 filed Dec. 21, 2018 entitled “Novel CRISPR Enzymes and Systems”, U.S. 62/712,809 filed Jul. 31, 2018 entitled “Novel CRISPR Enzymes and Systems”, U.S. 62/744,080 filed Oct. 10, 2018 entitled “Novel Cas12b Enzymes and Systems” and U.S. 62/751,196 filed Oct. 26 2018 entitled “Novel Cas12b Enzymes and Systems”, U.S. Pat. No. 715,640 filed August 7, 2-18 entitled “Novel CRISPR Enzymes and Systems”, WO 2016/205711, U.S. Pat. No. 9,790,490, WO 2016/205749, WO 2016/205764, WO 2017/070605, WO 2017/106657, and WO 2016/149661, WO2018/035387, WO2018/194963, Cox D B T, et al., RNA editing with CRISPR-Cas13, Science. 2017 Nov. 24; 358 (6366): 1019-1027; Gootenberg J S, et al., Multiplexed and portable nucleic acid detection platform with Cas13, Cas12a, and Csm6., Science. 2018 Apr. 27; 360 (6387): 439-444; Gootenberg J S, et al., Nucleic acid detection with CRISPR-Cas13a/C2c2., Science. 2017 Apr. 28; 356 (6336): 438-442; Abudayyeh O O, et al., RNA targeting with CRISPR-Cas13, Nature. 2017 Oct. 12; 550 (7675): 280-284; Smargon A A, et al., Cas13b Is a Type VI-B CRISPR-Associated RNA-Guided RNase Differentially Regulated by Accessory Proteins Csx27 and Csx28. Mol Cell. 2017 Feb. 16; 65 (4): 618-630.e7; Abudayyeh O O, et al., C2c2 is a single-component programmable RNA-guided RNA-targeting CRISPR effector, Science. 2016 Aug. 5; 353 (6299): aaf5573; Yang L, et al., Engineering and optimising deaminase fusions for genome editing. Nat Commun. 2016 Nov. 2; 7:13330, Myrvhold et al., Field deployable viral diagnostics using CRISPR-Cas13, Science 2018 360, 444-448, Shmakov et al. “Diversity and evolution of class 2 CRISPR-Cas systems,” Nat Rev Microbiol. 2017 15 (3): 169-182, each of which is incorporated herein by reference in its entirety.

### Nucleic Acid Barcode, Barcode, and Unique Molecular Identifier (UMI)

As previously described, the reporter polynucleotide can be or encode a barcode. The term “barcode” as used herein refers to a short sequence of nucleotides (for example, DNA or RNA) that is used as an identifier for an associated molecule, such as a target molecule and/or target nucleic acid, or as an identifier of the source of an associated molecule, such as a cell-of-origin. A barcode may also refer to any unique, non-naturally occurring, nucleic acid sequence that may be used to identify the originating source of a nucleic acid fragment. Although it is not necessary to understand the mechanism of an invention, it is believed that the barcode sequence provides a high-quality individual read of a barcode associated with a single cell, a viral vector, labeling ligand (e.g., an aptamer), protein, shRNA, sgRNA or cDNA such that multiple species can be sequenced together. In an embodiment, the barcode is a transcribable barcode.

Barcoding may be performed based on any of the compositions or methods disclosed in patent publication WO 2014047561 A1, Compositions and methods for labeling of agents, incorporated herein in its entirety. In certain embodiments barcoding uses an error correcting scheme (T. K. Moon, Error Correction Coding: Mathematical Methods and Algorithms (Wiley, New York, ed. 1, 2005)). Not being bound by a theory, amplified sequences from single cells can be sequenced together and resolved based on the barcode associated with each cell.

In preferred embodiments, sequencing is performed using unique molecular identifiers (UMI). The term “unique molecular identifiers” (UMI) as used herein refers to a sequencing linker or a subtype of nucleic acid barcode used in a method that uses molecular tags to detect and quantify unique amplified products. A UMI is used to distinguish effects through a single clone from multiple clones. The term “clone” as used herein may refer to a single mRNA or target nucleic acid to be sequenced. The UMI may also be used to determine the number of transcripts that gave rise to an amplified product, or in the case of target barcodes as described herein, the number of binding events. In preferred embodiments, the amplification is by PCR or multiple displacement amplification (MDA).

In certain embodiments, an UMI with a random sequence of between 4 and 20 base pairs is added to a template, which is amplified and sequenced. In preferred embodiments, the UMI is added to the 5′ end of the template. Sequencing allows for high resolution reads, enabling accurate detection of true variants. As used herein, a “true variant” will be present in every amplified product originating from the original clone as identified by aligning all products with a UMI. Each clone amplified will have a different random UMI that will indicate that the amplified product originated from that clone. Background caused by the fidelity of the amplification process can be eliminated because true variants will be present in all amplified products and background representing random error will only be present in single amplification products (See e.g., Islam S. et al., 2014. Nature Methods No: 11, 163-166). Not being bound by a theory, the UMI's are designed such that assignment to the original can take place despite up to 4-7 errors during amplification or sequencing. Not being bound by a theory, an UMI may be used to discriminate between true barcode sequences.

Unique molecular identifiers can be used, for example, to normalize samples for variable amplification efficiency. For example, in various embodiments, featuring a solid or semisolid support (for example a hydrogel bead), to which nucleic acid barcodes (for example a plurality of barcodes sharing the same sequence) are attached, each of the barcodes may be further coupled to a unique molecular identifier, such that every barcode on the particular solid or semisolid support receives a distinct unique molecule identifier. A unique molecular identifier can then be, for example, transferred to a target molecule with the associated barcode, such that the target molecule receives not only a nucleic acid barcode, but also an identifier unique among the identifiers originating from that solid or semisolid support.

A nucleic acid barcode or UMI can have a length of at least, for example, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 60, 70, 80, 90, or 100 nucleotides, and can be in single- or double-stranded form. Target molecule and/or target nucleic acids can be labeled with multiple nucleic acid barcodes in combinatorial fashion, such as a nucleic acid barcode concatemer. Typically, a nucleic acid barcode is used to identify a target molecule and/or target nucleic acid as being from a particular discrete volume, having a particular physical property (for example, affinity, length, sequence, etc.), or having been subject to certain treatment conditions. Target molecule and/or target nucleic acid can be associated with multiple nucleic acid barcodes to provide information about all of these features (and more). Each member of a given population of UMIs, on the other hand, is typically associated with (for example, covalently bound to or a component of the same molecule as) individual members of a particular set of identical, specific (for example, discreet volume-, physical property-, or treatment condition-specific) nucleic acid barcodes. Thus, for example, each member of a set of origin-specific nucleic acid barcodes, or other nucleic acid identifier or connector oligonucleotide, having identical or matched barcode sequences, may be associated with (for example, covalently bound to or a component of the same molecule as) a distinct or different UMI.

As disclosed herein, unique nucleic acid identifiers are used to label the target molecules and/or target nucleic acids, for example origin-specific barcodes and the like. The nucleic acid identifiers, nucleic acid barcodes, can include a short sequence of nucleotides that can be used as an identifier for an associated molecule, location, or condition. In certain embodiments, the nucleic acid identifier further includes one or more unique molecular identifiers and/or barcode receiving adapters. A nucleic acid identifier can have a length of about, for example, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 60, 70, 80, 90, or 100 base pairs (bp) or nucleotides (nt). In certain embodiments, a nucleic acid identifier can be constructed in combinatorial fashion by combining randomly selected indices (for example, about 1, 2, 3, 4, 5, 6, 7, 8, 9, or 10 indexes). Each such index is a short sequence of nucleotides (for example, DNA, RNA, or a combination thereof) having a distinct sequence. An index can have a length of about, for example, 4, 5, 6, 7, 8, 9, 10, 11, 12, 13, 14, 15, 16, 17, 18, 19, 20, 21, 22, 23, 24, or 25 bp or nt. Nucleic acid identifiers can be generated, for example, by split-pool synthesis methods, such as those described, for example, in International Patent Publication Nos. WO 2014/047556 and WO 2014/143158, each of which is incorporated by reference herein in its entirety.

One or more nucleic acid identifiers (for example a nucleic acid barcode) can be attached, or “tagged,” to a target molecule. This attachment can be direct (for example, covalent or noncovalent binding of the nucleic acid identifier to the target molecule) or indirect (for example, via an additional molecule). Such indirect attachments may, for example, include a barcode bound to a specific-binding agent that recognizes a target molecule. In certain embodiments, a barcode is attached to protein G and the target molecule is an antibody or antibody fragment. Attachment of a barcode to target molecules (for example, proteins and other biomolecules) can be performed using standard methods well known in the art. For example, barcodes can be linked via cysteine residues (for example, C-terminal cysteine residues). In other examples, barcodes can be chemically introduced into polypeptides (for example, antibodies) via a variety of functional groups on the polypeptide using appropriate group-specific reagents (see for example drmr.com/abcon). In certain embodiments, barcode tagging can occur via a barcode receiving adapter associate with (for example, attached to) a target molecule, as described herein.

Target molecules can be optionally labeled with multiple barcodes in combinatorial fashion (for example, using multiple barcodes bound to one or more specific binding agents that specifically recognizing the target molecule), thus greatly expanding the number of unique identifiers possible within a particular barcode pool. In certain embodiments, barcodes are added to a growing barcode concatemer attached to a target molecule, for example, one at a time. In other embodiments, multiple barcodes are assembled prior to attachment to a target molecule. Compositions and methods for concatemerization of multiple barcodes are described, for example, in International Patent Publication No. WO 2014/047561, which is incorporated herein by reference in its entirety.

In an embodiment, a nucleic acid identifier (for example, a nucleic acid barcode) may be attached to sequences that allow for amplification and sequencing (for example, SBS3 and P5 elements for Illumina sequencing). In certain embodiments, a nucleic acid barcode can further include a hybridization site for a primer (for example, a single-stranded DNA primer) attached to the end of the barcode. For example, an origin-specific barcode may be a nucleic acid including a barcode and a hybridization site for a specific primer. In particular embodiments, a set of origin-specific barcodes includes a unique primer specific barcode made, for example, using a randomized oligo type NNNNNNNNNNNN.

A nucleic acid identifier can further include a unique molecular identifier and/or additional barcodes specific to, for example, a common support to which one or more of the nucleic acid identifiers are attached. Thus, a pool of target molecules can be added, for example, to a discrete volume containing multiple solid or semisolid supports (for example, beads) representing distinct treatment conditions (and/or, for example, one or more additional solid or semisolid support can be added to the discreet volume sequentially after introduction of the target molecule pool), such that the precise combination of conditions to which a given target molecule was exposed can be subsequently determined by sequencing the unique molecular identifiers associated with it.

Labeled target molecules and/or target nucleic acids associated origin-specific nucleic acid barcodes (optionally in combination with other nucleic acid barcodes as described herein) can be amplified by methods known in the art, such as polymerase chain reaction (PCR). For example, the nucleic acid barcode can contain universal primer recognition sequences that can be bound by a PCR primer for PCR amplification and subsequent high-throughput sequencing. In certain embodiments, the nucleic acid barcode includes or is linked to sequencing adapters (for example, universal primer recognition sequences) such that the barcode and sequencing adapter elements are both coupled to the target molecule. In particular examples, the sequence of the origin specific barcode is amplified, for example using PCR. In an embodiment, an origin-specific barcode further comprises a sequencing adaptor. In an embodiment, an origin-specific barcode further comprises universal priming sites. A nucleic acid barcode (or a concatemer thereof), a target nucleic acid molecule (for example, a DNA or RNA molecule), a nucleic acid encoding a target peptide or polypeptide, and/or a nucleic acid encoding a specific binding agent may be optionally sequenced by any method known in the art, for example, methods of high-throughput sequencing, also known as next generation sequencing or deep sequencing. A nucleic acid target molecule labeled with a barcode (for example, an origin-specific barcode) can be sequenced with the barcode to produce a single read and/or contig containing the sequence, or portions thereof, of both the target molecule and the barcode. Exemplary next generation sequencing technologies include, for example, Illumina sequencing, Ion Torrent sequencing, 454 sequencing, SOLID sequencing, and nanopore sequencing amongst others. In an embodiment, the sequence of labeled target molecules is determined by non-sequencing based methods. For example, variable length probes or primers can be used to distinguish barcodes (for example, origin-specific barcodes) labeling distinct target molecules by, for example, the length of the barcodes, the length of target nucleic acids, or the length of nucleic acids encoding target polypeptides. In other instances, barcodes can include sequences identifying, for example, the type of molecule for a particular target molecule (for example, polypeptide, nucleic acid, small molecule, or lipid). For example, in a pool of labeled target molecules containing multiple types of target molecules, polypeptide target molecules can receive one identifying sequence, while target nucleic acid molecules can receive a different identifying sequence. Such identifying sequences can be used to selectively amplify barcodes labeling particular types of target molecules, for example, by using PCR primers specific to identifying sequences specific to particular types of target molecules. For example, barcodes labeling polypeptide target molecules can be selectively amplified from a pool, thereby retrieving only the barcodes from the polypeptide subset of the target molecule pool.

A nucleic acid barcode can be sequenced, for example, after cleavage, to determine the presence, quantity, or other feature of the target molecule. In certain embodiments, a nucleic acid barcode can be further attached to a further nucleic acid barcode. For example, a nucleic acid barcode can be cleaved from a specific-binding agent after the specific-binding agent binds to a target molecule or a tag (for example, an encoded polypeptide identifier element cleaved from a target molecule), and then the nucleic acid barcode can be ligated to an origin-specific barcode. The resultant nucleic acid barcode concatemer can be pooled with other such concatemers and sequenced. The sequencing reads can be used to identify which target molecules were originally present in which discrete volumes.

### Barcodes Reversibly Coupled to Solid Substrate

In an embodiment, the origin-specific barcodes are reversibly coupled to a solid or semisolid substrate. In an embodiment, the origin-specific barcodes further comprise a nucleic acid capture sequence that specifically binds to the target nucleic acids and/or a specific binding agent that specifically binds to the target molecules. In specific embodiments, the origin-specific barcodes include two or more populations of origin-specific barcodes, wherein a first population comprises the nucleic acid capture sequence and a second population comprises the specific binding agent that specifically binds to the target molecules. In some examples, the first population of origin-specific barcodes further comprises a target nucleic acid barcode, wherein the target nucleic acid barcode identifies the population as one that labels nucleic acids. In some examples, the second population of origin-specific barcodes further comprises a target molecule barcode, wherein the target molecule barcode identifies the population as one that labels target molecules.

Barcode with Cleavage Sites

A nucleic acid barcode may be cleavable from a specific binding agent, for example, after the specific binding agent has bound to a target molecule. In an embodiment, the origin-specific barcode further comprises one or more cleavage sites. In some examples, at least one cleavage site is oriented such that cleavage at that site releases the origin-specific barcode from a substrate, such as a bead, for example a hydrogel bead, to which it is coupled. In some examples, at least one cleavage site is oriented such that the cleavage at the site releases the origin-specific barcode from the target molecule specific binding agent. In some examples, a cleavage site is an enzymatic cleavage site, such an endonuclease site present in a specific nucleic acid sequence. In other embodiments, a cleavage site is a peptide cleavage site, such that a particular enzyme can cleave the amino acid sequence. In still other embodiments, a cleavage site is a site of chemical cleavage.

### Barcode Adapters

In an embodiment, the target molecule is attached to an origin-specific barcode receiving adapter, such as a nucleic acid. In some examples, the origin-specific barcode receiving adapter comprises an overhang and the origin-specific barcode comprises a sequence capable of hybridizing to the overhang. A barcode receiving adapter is a molecule configured to accept or receive a nucleic acid barcode, such as an origin-specific nucleic acid barcode. For example, a barcode receiving adapter can include a single-stranded nucleic acid sequence (for example, an overhang) capable of hybridizing to a given barcode (for example, an origin-specific barcode), for example, via a sequence complementary to a portion or the entirety of the nucleic acid barcode. In certain embodiments, this portion of the barcode is a standard sequence held constant between individual barcodes. The hybridization couples the barcode receiving adapter to the barcode. In an embodiment, the barcode receiving adapter may be associated with (for example, attached to) a target molecule. As such, the barcode receiving adapter may serve as the means through which an origin-specific barcode is attached to a target molecule. A barcode receiving adapter can be attached to a target molecule according to methods known in the art. For example, a barcode receiving adapter can be attached to a polypeptide target molecule at a cysteine residue (for example, a C-terminal cysteine residue). A barcode receiving adapter can be used to identify a particular condition related to one or more target molecules, such as a cell of origin or a discreet volume of origin. For example, a target molecule can be a cell surface protein expressed by a cell, which receives a cell-specific barcode receiving adapter. The barcode receiving adapter can be conjugated to one or more barcodes as the cell is exposed to one or more conditions, such that the original cell of origin for the target molecule, as well as each condition to which the cell was exposed, can be subsequently determined by identifying the sequence of the barcode receiving adapter/barcode concatemer.

Barcode with Capture Moiety

In an embodiment, an origin-specific barcode further includes a capture moiety, covalently or non-covalently linked. Thus, In an embodiment the origin-specific barcode, and anything bound or attached thereto, that include a capture moiety are captured with a specific binding agent that specifically binds the capture moiety. In an embodiment, the capture moiety is adsorbed or otherwise captured on a surface. In specific embodiments, a targeting probe is labeled with biotin, for instance by incorporation of biotin-16-UTP during in vitro transcription, allowing later capture by streptavidin. Other means for labeling, capturing, and detecting an origin-specific barcode include: incorporation of aminoallyl-labeled nucleotides, incorporation of sulfhydryl-labeled nucleotides, incorporation of allyl- or azide-containing nucleotides, and many other methods described in Bioconjugate Techniques (2^{nd} Ed), Greg T. Hermanson, Elsevier (2008), which is specifically incorporated herein by reference. In an embodiment, the targeting probes are covalently coupled to a solid support or other capture device prior to contacting the sample, using methods such as incorporation of aminoallyl-labeled nucleotides followed by 1-Ethyl-3-(3-dimethylaminopropyl) carbodiimide (EDC) coupling to a carboxy-activated solid support, or other methods described in Bioconjugate Techniques. In an embodiment, the specific binding agent has been immobilized for example on a solid support, thereby isolating the origin-specific barcode.

### Other Barcoding Embodiments

DNA barcoding is also a taxonomic method that uses a short genetic marker in an organism's DNA to identify it as belonging to a particular species. It differs from molecular phylogeny in that the main goal is not to determine classification but to identify an unknown sample in terms of a known classification. Kress et al., “Use of DNA barcodes to identify flowering plants” Proc. Natl. Acad. Sci. U.S.A. 102 (23): 8369-8374 (2005). Barcodes are sometimes used in an effort to identify unknown species or assess whether species should be combined or separated. Koch H., “Combining morphology and DNA barcoding resolves the taxonomy of Western Malagasy Liotrigona Moure, 1961” African Invertebrates 51 (2): 413-421 (2010); and Seberg et al., “How many loci does it take to DNA barcode a crocus?” PLOS One 4 (2):e4598 (2009). Barcoding has been used, for example, for identifying plant leaves even when flowers or fruit are not available, identifying the diet of an animal based on stomach contents or feces, and/or identifying products in commerce (for example, herbal supplements or wood). Soininen et al., “Analysing diet of small herbivores: the efficiency of DNA barcoding coupled with high-throughput pyrosequencing for deciphering the composition of complex plant mixtures” Frontiers in Zoology 6:16 (2009).

It has been suggested that a desirable locus for DNA barcoding should be standardized so that large databases of sequences for that locus can be developed. Most of the taxa of interest have loci that are sequenceable without species-specific PCR primers. CBOL Plant Working Group, “A DNA barcode for land plants” PNAS 106 (31): 12794-12797 (2009). Further, these putative barcode loci are believed short enough to be easily sequenced with current technology. Kress et al., “DNA barcodes: Genes, genomics, and bioinformatics” PNAS 105 (8): 2761-2762 (2008). Consequently, these loci would provide a large variation between species in combination with a relatively small amount of variation within a species. Lahaye et al., “DNA barcoding the floras of biodiversity hotspots” Proc Natl Acad Sci USA 105 (8): 2923-2928 (2008).

DNA barcoding is based on a relatively simple concept. For example, most eukaryote cells contain mitochondria, and mitochondrial DNA (mtDNA) has a relatively fast mutation rate, which results in significant variation in mtDNA sequences between species and, in principle, a comparatively small variance within species. A 648-bp region of the mitochondrial cytochrome c oxidase subunit 1 (CO1) gene was proposed as a potential ‘barcode’. As of 2009, databases of CO1 sequences included at least 620,000 specimens from over 58,000 species of animals, larger than databases available for any other gene. Ausubel, J., “A botanical macroscope” Proceedings of the National Academy of Sciences 106 (31): 12569 (2009).

Software for DNA barcoding requires integration of a field information management system (FIMS), laboratory information management system (LIMS), sequence analysis tools, workflow tracking to connect field data and laboratory data, database submission tools and pipeline automation for scaling up to eco-system scale projects. Geneious Pro can be used for the sequence analysis components, and the two plugins made freely available through the Moorea Biocode Project, the Biocode LIMS and Genbank Submission plugins handle integration with the FIMS, the LIMS, workflow tracking and database submission.

Additionally, other barcoding designs and tools have been described (see e.g., Birrell et al., (2001) Proc. Natl Acad. Sci. USA 98, 12608-12613; Giaever, et al., (2002) Nature 418, 387-391; Winzeler et al., (1999) Science 285, 901-906; and Xu et al., (2009) Proc Natl Acad Sci USA. February 17; 106 (7): 2289-94).

### Vectors and Vector Systems

Described in certain example embodiments herein are vector systems comprising one or more vectors comprising one or more CREs of the present invention and/or one or more engineered polynucleotides of the present invention previously described.

In certain embodiments, the vector can contain one or more polynucleotides encoding one or more vectors comprising one or more CREs of the present invention and/or one or more engineered polynucleotides of the present invention previously described. The vectors can be useful in producing bacterial, fungal, yeast, plant cells, animal cells, and/or transgenic and/or otherwise modified organisms as described herein. Within the scope of this disclosure are vectors containing one or more of the polynucleotide sequences described herein. The vectors and/or vector systems can be used, for example, to express one or more polynucleotides in a cell types, cell state, tissue type, or environment specific manner. In an embodiment, expression of the vector or vector system is in a producer cell, so as to produce one or more gene products that can be expressed from the polynucleotide of the engineered polynucleotide of the present invention. In an embodiment, the producer cell produces virus particles, virus like particles or a non-viral delivery vesicle (e.g., an exosome) that contains an engineered polynucleotide and/or gene product encoded by the polynucleotide component of the engineered polynucleotide of the present invention described elsewhere herein. Other uses for the vectors and vector systems described herein are also within the scope of this disclosure. In general, and throughout this specification, the term “vector” refers to a tool that allows or facilitates the transfer of an entity from one environment to another. In some contexts which will be appreciated by those of ordinary skill in the art, “vector” can be a term of art to refer to a nucleic acid molecule capable of transporting another nucleic acid to which it has been linked. A vector can be a replicon, such as a plasmid, phage, or cosmid, into which another DNA segment may be inserted so as to bring about the replication of the inserted segment. Generally, a vector is capable of replication when associated with the proper control elements.

Vectors include, but are not limited to, nucleic acid molecules that are single-stranded, double-stranded, or partially double-stranded; nucleic acid molecules that comprise one or more free ends, no free ends (e.g., circular); nucleic acid molecules that comprise DNA, RNA, or both; and other varieties of polynucleotides known in the art. One type of vector is a “plasmid,” which refers to a circular double-stranded DNA loop into which additional DNA segments can be inserted, such as by standard molecular cloning techniques. Another type of vector is a viral vector, wherein virally-derived DNA or RNA sequences are present in the vector for packaging into a virus (e.g. retroviruses, replication defective retroviruses, adenoviruses, replication defective adenoviruses, and adeno-associated viruses (AAVs)). Viral vectors also include polynucleotides carried by a virus for transfection into a host cell. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell and thereby are replicated along with the host genome. Moreover, certain vectors are capable of directing the expression of genes to which they are operatively-linked. Such vectors are referred to herein as “expression vectors.” Common expression vectors of utility in recombinant DNA techniques are often in the form of plasmids.

Recombinant expression vectors can be composed of a nucleic acid (e.g., a polynucleotide) of the invention in a form suitable for expression of the nucleic acid in a host cell, which means that the recombinant expression vectors include one or more regulatory elements, which can be selected on the basis of the host cells to be used for expression, that is operatively-linked to the nucleic acid sequence to be expressed. Within a recombinant expression vector, “operably linked” and “operatively-linked” are used interchangeably herein and further defined elsewhere herein. In the context of a vector, the term “operably linked” is intended to mean that the nucleotide sequence of interest is linked to the regulatory element(s) in a manner that allows for expression of the nucleotide sequence (e.g., in an in vitro transcription/translation system or in a host cell when the vector is introduced into the host cell). Advantageous vectors include lentiviruses and adeno-associated viruses, and types of such vectors can also be selected for targeting particular types of cells. These and other embodiments of the vectors and vector systems are described elsewhere herein.

In an embodiment, the vector can be a bicistronic vector. In an embodiment, a bicistronic vector comprises one or more CREs of the present invention and/or one or more engineered polynucleotides of the present invention. In an embodiment, a bicistronic vector can be used for one or more engineered polynucleotides described herein. In an embodiment, in addition to or more CREs of the present invention, expression of element(s) of the engineered polynucleotide of the present invention are driven or otherwise regulated by a ubiquitous Pol II promoter, such as beta-actin, CMV, SV40, or another ubiquitous promoter. In an embodiment, in addition to or more CREs of the present invention, expression of element(s) of the engineered polynucleotide of the present invention are driven or otherwise regulated by a tissue-specific Pol II promoter. Where the polynucleotide element of the engineered polynucleotide is an RNA, in addition to one or more CREs of the present invention its expression can be driven by a Pol III promoter, such as a U6 promoter. In an embodiment, the two are combined.

These and others are further detailed and described elsewhere herein.

### Cell-Based Vector Amplification and Expression

Vectors may be introduced and propagated in a prokaryote or prokaryotic cell. In an embodiment, a prokaryote is used to amplify copies of a vector to be introduced into a eukaryotic cell or as an intermediate vector in the production of a vector to be introduced into a eukaryotic cell (e.g., amplifying a plasmid as part of a viral vector packaging system). The vectors can be viral-based or non-viral based. In an embodiment, a prokaryote is used to amplify copies of a vector and express one or more nucleic acids, such as to provide a source of one or more proteins for delivery to a host cell or host organism.

Vectors can be designed for expression of one or more elements of the engineered polynucleotides of the present invention or component thereof described herein (e.g., nucleic acid transcripts, proteins, enzymes, and combinations thereof) in a suitable host cell. In an embodiment, the suitable host cell is a prokaryotic cell. Suitable host cells include, but are not limited to, bacterial cells, yeast cells, insect cells, and mammalian cells. In an embodiment, the suitable host cell is a eukaryotic cell.

In an embodiment, the suitable host cell is a suitable bacterial cell. Suitable bacterial cells include, but are not limited to, bacterial cells from the bacteria of the species Escherichia coli. Many suitable strains of E. coli are known in the art for expression of vectors. These include, but are not limited to Pir1, Stb12, Stb13, Stb14, TOP10, XL1 Blue, XL10 Gold, Rosetta 2 (DE3) (Novagen), NEB® 5-alpha Competent E. coli (High Efficiency) (New England Biolabs), and BL21 (DE3) Competent E. coli (New England Biolabs). In an embodiment, the host cell is a suitable insect cell. Suitable insect cells include those from Spodoptera frugiperda. Suitable strains of S. frugiperda cells include, but are not limited to, Sf9 and Sf21. In an embodiment, the host cell is a suitable yeast cell. In an embodiment, the yeast cell can be from Saccharomyces cerevisiae. In an embodiment, the host cell is a suitable mammalian cell. Many types of mammalian cells have been developed to express vectors. Suitable mammalian cells include, but are not limited to, HEK293, HEK293T, HEK293FT, Chinese Hamster Ovary Cells (CHOs), mouse myeloma cells, HeLa, U2OS, A549, HT1080, CAD, P19, NIH 3T3, L929, N2a, MCF-7, Y79, SO-Rb50, HepG G2, DIKX-X11, J558L, Baby hamster kidney cells (BHK), and chicken embryo fibroblasts (CEFs). Suitable host cells are discussed further in Goeddel, GENE EXPRESSION TECHNOLOGY: METHODS IN ENZYMOLOGY 185, Academic Press, San Diego, Calif. (1990).

In an embodiment, the vector can be a yeast expression vector. Examples of vectors for expression in yeast Saccharomyces cerevisiae include pYepSec1 (Baldari, et al., 1987. EMBO J. 6:229-234), pMFa (Kuijan and Herskowitz, 1982. Cell 30:933-943), pJRY88 (Schultz et al., 1987. Gene 54:113-123), pYES2 (Invitrogen Corporation, San Diego, Calif.), and picZ (InVitrogen Corp, San Diego, Calif.). As used herein, a “yeast expression vector” refers to a nucleic acid that contains one or more sequences encoding an RNA and/or polypeptide and may further contain any desired elements that control the expression of the nucleic acid(s), as well as any elements that enable the replication and maintenance of the expression vector inside the yeast cell. Many suitable yeast expression vectors and features thereof are known in the art; for example, various vectors and techniques are illustrated in Yeast Protocols, 2^{nd} edition, Xiao, W., ed. (Humana Press, New York, 2007) and Buckholz, R. G. and Gleeson, M. A. (1991) Biotechnology (NY) 9 (11): 1067-72. Yeast vectors can contain, without limitation, a centromeric (CEN) sequence, an autonomous replication sequence (ARS), a promoter, such as an RNA Polymerase III promoter, operably linked to a sequence or gene of interest, a terminator such as an RNA polymerase III terminator, an origin of replication, and a marker gene (e.g., auxotrophic, antibiotic, or other selectable markers). Examples of expression vectors for use in yeast may include plasmids, yeast artificial chromosomes, 2μ plasmids, yeast integrative plasmids, yeast replicative plasmids, shuttle vectors, and episomal plasmids.

In an embodiment, the vector is a baculovirus vector or expression vector and can be suitable for expression of polynucleotides and/or proteins in insect cells. In an embodiment, the suitable host cell is an insect cell. Baculovirus vectors available for expression of proteins in cultured insect cells (e.g., SF9 cells) include the pAc series (Smith, et al., 1983. Mol. Cell. Biol. 3:2156-2165) and the pVL series (Lucklow and Summers, 1989. Virology 170:31-39). rAAV (recombinant Adeno-associated viral) vectors are preferably produced in insect cells, e.g., Spodoptera frugiperda Sf9 insect cells, grown in serum-free suspension culture. Serum-free insect cells can be purchased from commercial vendors, e.g., Sigma Aldrich (EX-CELL 405).

In an embodiment, the vector is a mammalian expression vector. In an embodiment, the mammalian expression vector is capable of expressing one or more polynucleotides and/or polypeptides in a mammalian cell. Examples of mammalian expression vectors include, but are not limited to, pCDM8 (Seed, 1987. Nature 329:840) and pMT2PC (Kaufman, et al., 1987. EMBO J. 6:187-195). The mammalian expression vector can include one or more suitable regulatory elements capable of controlling expression of the one or more polynucleotides and/or proteins in the mammalian cell. For example, commonly used promoters are derived from polyoma, adenovirus 2, cytomegalovirus, simian virus 40, and others disclosed herein and known in the art. More details on suitable regulatory elements are described elsewhere herein.

For other suitable expression vectors and vector systems for both prokaryotic and eukaryotic cells see, e.g., Chapters 16 and 17 of Sambrook, et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

In an embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert, et al., 1987. Genes Dev. 1:268-277), lymphoid-specific promoters (Calame and Eaton, 1988. Adv. Immunol. 43:235-275), in particular promoters of T cell receptors (Winoto and Baltimore, 1989. EMBO J. 8:729-733) and immunoglobulins (Baneiji, et al., 1983. Cell 33:729-740; Queen and Baltimore, 1983. Cell 33:741-748), neuron-specific promoters (e.g., the neurofilament promoter; Byrne and Ruddle, 1989. Proc. Natl. Acad. Sci. USA 86:5473-5477), pancreas-specific promoters (Edlund, et al., 1985. Science 230:912-916), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel and Gruss, 1990. Science 249:374-379) and the α-fetoprotein promoter (Campes and Tilghman, 1989. Genes Dev. 3:537-546). With regards to these prokaryotic and eukaryotic vectors, mention is made of U.S. Pat. No. 6,750,059, the contents of which are incorporated by reference herein in their entirety. Other embodiments can utilize viral vectors, with regards to which mention is made of U.S. patent application Ser. No. 13/092,085, the contents of which are incorporated by reference herein in their entirety. Tissue-specific regulatory elements are known in the art and in this regard, mention is made of U.S. Pat. No. 7,776,321, the contents of which are incorporated by reference herein in their entirety. In an embodiment, a regulatory element including but not limited to one or more CREs of the present invention can be operably linked to one or more elements of the engineered polynucleotide (such as one or more polynucleotide components) so as to drive, inhibit, or otherwise regulate expression of the one or more elements of the engineered polynucleotide of the present invention.

In an embodiment, the vector can be a fusion vector or fusion expression vector. In an embodiment, fusion vectors add a number of amino acids to a protein encoded therein, such as to the amino terminus, carboxy terminus, or both of a recombinant protein. Such fusion vectors can serve one or more purposes, such as: (i) to increase expression of recombinant protein; (ii) to increase the solubility of the recombinant protein; and (iii) to aid in the purification of the recombinant protein by acting as a ligand in affinity purification. In an embodiment, expression of polynucleotides (such as non-coding polynucleotides) and proteins in prokaryotes can be carried out in Escherichia coli with vectors containing constitutive or inducible promoters directing the expression of either fusion or non-fusion polynucleotides and/or proteins. In an embodiment, the fusion expression vector can include one or more proteolytic cleavage sites, which can be introduced at the junction of the fusion vector backbone or other fusion moiety and the recombinant polynucleotide or protein to enable separation of the recombinant polynucleotide or protein from the fusion vector backbone or other fusion moiety subsequent to purification of the fusion polynucleotide or protein. Such enzymes, and their cognate recognition sequences, include Factor Xa, thrombin and enterokinase, and TEV protease sites. Example fusion expression vectors include pGEX (Pharmacia Biotech Inc; Smith and Johnson, 1988. Gene 67:31-40), pMAL (New England Biolabs, Beverly, Mass.) and pRIT5 (Pharmacia, Piscataway, N.J.) that fuse glutathione S-transferase (GST), maltose-binding protein, or protein A, respectively, to the target recombinant protein. Examples of suitable inducible non-fusion E. coli expression vectors include pTrc (Amrann et al., (1988) Gene 69:301-315) and pET 11d (Studier et al., GENE EXPRESSION TECHNOLOGY: METHODS IN ENZYMOLOGY 185, Academic Press, San Diego, Calif. (1990) 60-89).

In an embodiment, one or more vectors described herein are introduced into a host cell such that expression of the engineered polynucleotides or components thereof described herein direct formation of a gene product complex in one or more cells, such as one or more specific cell types, cell states, tissue types or cells within a specific environment in which the one or more CREs are specific for.

In an embodiment, two or more polynucleotide elements of the engineered polynucleotides of the present invention can be expressed and/or otherwise regulated from the same or different regulatory element(s) (including but not limited to one or more CREs of the present invention), can be combined in a single vector, with one or more additional vectors providing any components of the system not included in the first vector. Engineered polynucleotides and/or multiple polynucleotide elements that are combined in a single vector may be arranged in any suitable orientation, such as one element located 5′ with respect to (“upstream” of) or 3′ with respect to (“downstream” of) a second element. The coding sequence of one element may be located on the same or opposite strand of the coding sequence of a second element, and oriented in the same or opposite direction. In an embodiment, a single promoter, optionally a CRE of the present invention, drives expression of a transcript embedded within one or more intron sequences (e.g., each in a different intron, two or more in at least one intron, or all in a single intron).

### Cell-Free Vector and Polynucleotide Expression

In an embodiment, one or more CREs and/or one or more engineered polynucleotides and/or component thereof (e.g., a polynucleotide component) of the present invention is included in and optionally expressed by a vector or suitable polynucleotide in a cell-free in vitro system. In other words, the one or more polynucleotide components of the engineered polynucleotide or vector can be transcribed and optionally translated in vitro. In some such embodiments, the CREs can be specific for one or more environment conditions that can be present (or not) in an in vitro, cell-free system. In vitro transcription/translation systems and appropriate vectors are generally known in the art and commercially available. Generally, in vitro transcription and in vitro translation systems replicate the processes of RNA and protein synthesis, respectively, outside of the cellular environment. Vectors and suitable polynucleotides for in vitro transcription can include T7, SP6, and T3 promoters or other regulatory sequences that in addition to the CREs of the present invention can be recognized and acted upon by an appropriate polymerase to transcribe the polynucleotide or one or more regions of a vector.

In vitro translation can be stand-alone (e.g., translation of a purified polyribonucleotide) or linked/coupled to transcription. In an embodiment, the cell-free (or in vitro) translation system can include extracts from rabbit reticulocytes, wheat germ, and/or E. coli. The extracts can include various macromolecular components that are needed for translation of exogenous RNA (e.g., 70S or 80S ribosomes, tRNAs, aminoacyl-tRNA, synthetases, initiation, elongation factors, termination factors, etc.). Other components can be included or added during the translation reaction, including but not limited to, amino acids, energy sources (ATP, GTP), energy regenerating systems (e.g., creatine phosphate and creatine phosphokinase for use in eukaryotic systems) and phosphoenol pyruvate and pyruvate kinase for use in bacterial systems), and other co-factors (e.g., Mg²⁺, K⁺, etc.). As previously mentioned, in vitro translation can be based on RNA or DNA starting material. Some translation systems can utilize an RNA template as starting material (e.g., reticulocyte lysates and wheat germ extracts). Some translation systems can utilize a DNA template as a starting material (e.g., E coli-based systems). In these systems, transcription and translation are coupled and DNA is first transcribed into RNA, which is subsequently translated. Suitable standard and coupled cell-free translation systems are generally known in the art and are commercially available.

### Vector Features

The vectors can include additional features that can confer one or more functionalities to the vector, the polynucleotide to be delivered, a virus particle produced there from, or polypeptide expressed thereof. Such features include, but are not limited to, regulatory elements, selectable markers, molecular identifiers (e.g. molecular barcodes), stabilizing elements, and the like. It will be appreciated by those skilled in the art that the design of the expression vector and additional features included can depend on such factors as the choice of the host cell to be transformed, the level of expression desired, etc.

### Regulatory Elements

In certain embodiments, the polynucleotides and/or vectors thereof described herein of the present invention can include one or more regulatory elements that can be operatively linked to the polynucleotide. In an embodiment, the regulatory element is one or more CREs of the present invention. In an embodiment, one or more additional regulatory elements can be operatively coupled to the one or more polynucleotide components of the engineered polynucleotide and/or CREs of the present invention. The term “regulatory element” is intended to include promoters, enhancers, internal ribosomal entry sites (IRES), other expression control elements (e.g., transcription termination signals, such as polyadenylation signals and poly-U sequences) and cellular localization signals (e.g. nuclear localization signals). Such regulatory elements are described, for example, in Goeddel, GENE EXPRESSION TECHNOLOGY: METHODS IN ENZYMOLOGY 185, Academic Press, San Diego, Calif. (1990). Regulatory elements include those that direct constitutive expression of a nucleotide sequence in many types of host cells and those that direct expression of the nucleotide sequence only in certain host cells (e.g., tissue-specific regulatory sequences). A tissue-specific promoter can direct expression primarily in a desired tissue of interest, such as muscle, neuron, bone, skin, blood, specific organs (e.g., liver, pancreas), or particular cell types (e.g., lymphocytes). Regulatory elements may also direct expression in a temporal-dependent manner, such as in a cell cycle-dependent or developmental stage-dependent manner, which may or may not also be tissue or cell-type specific. In an embodiment, a vector comprises one or more pol III promoters (e.g., 1, 2, 3, 4, 5, or more pol III promoters), one or more pol II promoters (e.g., 1, 2, 3, 4, 5, or more pol II promoters), one or more pol I promoters (e.g., 1, 2, 3, 4, 5, or more pol I promoters), or combinations thereof. Examples of pol III promoters include, but are not limited to, U6 and H1 promoters. Examples of pol II promoters include, but are not limited to, the retroviral Rous sarcoma virus (RSV) LTR promoter (optionally with the RSV enhancer), the cytomegalovirus (CMV) promoter (optionally with the CMV enhancer) (see, e.g., Boshart et al, Cell, 41:521-530 (1985)), the SV40 promoter, the dihydrofolate reductase promoter, the β-actin promoter, the phosphoglycerol kinase (PGK) promoter, and the EF1α promoter. Also encompassed by the term “regulatory element” are enhancer elements, such as woodchuck hepatitis virus post-transcriptional regulator element (WPRE); CMV enhancers; the R-U5′ segment in the long terminal repeat (LTR) of HTLV-I (Mol. Cell. Biol., Vol. 8 (1), p. 466-472, 1988); SV40 enhancer; and the intron sequence between exons 2 and 3 of rabbit β-globin (Proc. Natl. Acad. Sci. USA., Vol. 78 (3), p. 1527-31, 1981).

In an embodiment, the regulatory sequence can be a regulatory sequence described in U.S. Pat. No. 7,776,321, U.S. Pat. Pub. No. 2011/0027239, and International Patent Publication No. WO 2011/028929, the contents of which are incorporated by reference herein in their entirety. In an embodiment, the vector can contain a minimal promoter. In an embodiment, the minimal promoter is the Mecp2 promoter, tRNA promoter, or U6. In a further embodiment, the minimal promoter is tissue specific. In an embodiment, the length of the vector polynucleotide, the minimal promoters, and polynucleotide sequences is less than 4.4 kb.

To express a polynucleotide, the vector can include one or more transcriptional and/or translational initiation regulatory sequences, e.g. promoters, that direct the transcription of the gene and/or translation of the encoded protein in a cell. In an embodiment, a constitutive promoter may be employed. Suitable constitutive promoters for mammalian cells are generally known in the art and include, but are not limited to SV40, CAG, CMV, EF-1α, β-actin, RSV, and PGK. Suitable constitutive promoters for bacterial cells, yeast cells, and fungal cells are generally known in the art, such as a T7 promoter for bacterial expression and an alcohol dehydrogenase promoter for expression in yeast.

In an embodiment, the regulatory element can be a regulated promoter. “Regulated promoter” refers to promoters that direct gene expression not constitutively, but in a temporally- and/or spatially-regulated manner, and includes tissue-specific, tissue-preferred, and inducible promoters. Regulated promoters include conditional promoters and inducible promoters. In an embodiment, conditional promoters can be employed to direct expression of a polynucleotide in a specific cell type, under certain environmental conditions, and/or during a specific state of development. Suitable tissue-specific promoters can include, but are not limited to, liver-specific promoters (e.g. APOA2, SERPIN A1 (hAAT), CYP3A4, and MIR122), pancreatic cell promoters (e.g. INS, IRS2, Pdx1, Alx3, Ppy), cardiac-specific promoters (e.g. Myh6 (alpha MHC), MYL2 (MLC-2v), TNI3 (cTnl), NPPA (ANF), Slc8al (Ncx1)), central nervous system cell promoters (SYN1, GFAP, INA, NES, MOBP, MBP, TH, FOXA2 (HNF3 beta)), skin cell-specific promoters (e.g. FLG, K14, TGM3), immune cell-specific promoters, (e.g. ITGAM, CD43 promoter, CD14 promoter, CD45 promoter, CD68 promoter), urogenital cell-specific promoters (e.g. Pbsn, Upk2, Sbp, Fer114), endothelial cell-specific promoters (e.g. ENG), pluripotent and embryonic germ layer cell-specific promoters (e.g. Oct4, NANOG, Synthetic Oct4, T brachyury, NES, SOX17, FOXA2, MIR122), and muscle cell-specific promoter (e.g. Desmin). Other tissue and/or cell-specific promoters are generally known in the art and are within the scope of this disclosure.

Inducible/conditional promoters can be positively inducible/conditional promoters (e.g. a promoter that activates transcription of the polynucleotide upon appropriate interaction with an activated activator, or an inducer compound, environmental condition, or another stimulus) or a negative/conditional inducible promoter (e.g. a promoter that is repressed by e.g., being bound by a repressor) until the repressor condition of the promotor is removed e.g., when inducer binds a repressor bound to the promoter, stimulating release of the promoter by the repressor or removal of a chemical repressor from the promoter environment. The inducer can be a compound, environmental condition, or another stimulus. Thus, inducible/conditional promoters can be responsive to any suitable stimuli such as chemical, biological, or other molecular agents, temperature, light, and/or pH. Suitable inducible/conditional promoters include, but are not limited to, Tet-On, Tet-Off, Lac promoter, pBad, AlcA, LexA, Hsp70 promoter, Hsp90 promoter, pDawn, XVE/OlexA, GVG, and pOp/LhGR.

Where expression in a plant cell is desired, engineered polynucleotide and/or vector described herein include one or more plant cell specific regulatory elements, including but not limited to one or more e.g., plant cell type specific, plant cell state specific, plant tissue type specific CREs, and/or other regulatory elements, such as a plant promoter, i.e. a promoter operable in plant cells. The use of different types of promoters is envisaged as is further described elsewhere herein.

A constitutive plant promoter is a promoter that is able to express the open reading frame (ORF) that it controls in all or nearly all of the plant tissues during all or nearly all developmental stages of the plant (referred to as “constitutive expression”). In an embodiment, one or more CREs of the present invention is a plant cell specific constitutive promoter. Another non-limiting example of a constitutive promoter is the cauliflower mosaic virus 35S promoter. Different promoters may direct the expression of a gene in different tissues or cell types, or at different stages of development, or in response to different environmental conditions. In an embodiment, one or more CREs of the present invention are Examples of particular plant promoters that can be included in the vectors described herein are found in Kawamata et al., (1997) Plant Cell Physiol 38:792-803; Yamamoto et al., (1997) Plant J 12:255-65; Hire et al, (1992) Plant Mol Biol 20:207-18, Kuster et al, (1995) Plant Mol Biol 29:759-72, and Capana et al., (1994) Plant Mol Biol 25:681-91.

In an embodiment, the vector includes one or more promoters or other regulatory elements that are inducible and that can allow for spatiotemporal control of polynucleotide expression may use a form of energy. In an embodiment, one or more CREs of the present invention have activity under certain environment conditions, such as exposure to a form of energy. Examples of other promoters that are inducible and that can allow for spatiotemporal control of polynucleotide expression may use a form of energy. The form of energy may include but is not limited to sound energy, electromagnetic radiation, chemical energy, and/or thermal energy. Examples of inducible systems include tetracycline-inducible promoters (Tet-On or Tet-Off), small molecule two-hybrid transcription activation systems (FKBP, ABA, etc.), or light-inducible systems (Phytochrome, Light-oxygen-voltage-sensing (LOV) domains, or cryptochrome, such as a Light Inducible Transcriptional Effector (LITE) that directs changes in transcriptional activity in a sequence-specific manner. The components of a light-inducible system may include a light-responsive cytochrome heterodimer (e.g., from Arabidopsis thaliana), and a transcriptional activation/repression domain. In an embodiment, the vector can include one or more of the inducible DNA binding proteins provided in International Patent Publication No. WO 2014/018423 and US Patent Publication Nos., 2015/0291966, 2017/0166903, 2019/0203212, which describe e.g., embodiments of inducible DNA binding proteins and methods of use and can be adapted for use with the present invention.

In an embodiment, transient or inducible expression can be achieved by including, for example, chemical-regulated promoters or other regulatory elements, i.e., whereby the application of an exogenous chemical induces gene expression. In an embodiment, one or more CREs of the present invention have activity under certain environment conditions, such as exposure to a particular chemical. Other chemically responsive promoters and other regulatory elements known in the art can also be included in the engineered polynucleotide and/or vectors described herein. In an embodiment, response to the chemical is to repress or activate or polynucleotide expression Exemplary known chemical-inducible promoters include, but are not limited to, the maize In2-2 promoter, activated by benzene sulfonamide herbicide safeners (De Veylder et al., (1997) Plant Cell Physiol 38:568-77), the maize GST promoter (GST-11-27, WO93/01294), activated by hydrophobic electrophilic compounds used as pre-emergent herbicides, and the tobacco PR-1a promoter (Ono et al., (2004) Biosci Biotechnol Biochem 68:803-7) activated by salicylic acid. Promoters that are regulated by antibiotics, such as tetracycline-inducible and tetracycline-repressible promoters (Gatz et al., (1991) Mol Gen Genet 227:229-37; U.S. Pat. Nos. 5,814,618 and 5,789,156) can also be used herein.

In an embodiment, the polynucleotide, vector or system thereof can include one or more elements capable of translocating and/or expressing an engineered polynucleotide or component thereof (e.g., a non-CRE polynucleotide component) to/in a specific cell component or organelle. Such organelles can include, but are not limited to, nucleus, ribosome, endoplasmic reticulum, Golgi apparatus, chloroplast, mitochondria, vacuole, lysosome, cytoskeleton, plasma membrane, cell wall, peroxisome, centrioles, etc. Such regulatory elements can include, but are not limited to, nuclear localization signals (examples of which are described in greater detail elsewhere herein), any such as those that are annotated in the LocSigDB database (see e.g., Negi et al., 2015. Database. 2015: bav003; doi: 10.1093/database/bav003), nuclear export signals (e.g., LXXXLXXLXL (SEQ ID NO: 20) and others described elsewhere herein), endoplasmic reticulum localization/retention signals (e.g. KDEL (SEQ ID NO: 21), KDXX, KKXX, KXX, and others described elsewhere herein; and see e.g. Liu et al. 2007 Mol. Biol. Cell. 18 (3): 1073-1082 and Gorleku et al., 2011. J. Biol. Chem. 286:39573-39584), mitochondria (see e.g. Cell Reports. 22:2818-2826, particularly at FIG. 2; Doyle et al. 2013. PLOS ONE 8, e67938; Funes et al. 2002. J. Biol. Chem. 277:6051-6058; Matouschek et al. 1997. PNAS USA 85:2091-2095; Oca-Cossio et al., 2003. 165:707-720; Waltner et al., 1996. J. Biol. Chem. 271:21226-21230; Wilcox et al., 2005. PNAS USA 102:15435-15440; Galanis et al., 1991. FEBS Lett 282:425-430), peroxisome (e.g. (S/A/C)-(K/R/H)-(L/A), SLK, (R/K)-(L/V/I)-XXXXX-(H/Q)-(L/A/F). Suitable protein targeting motifs can also be designed or identified using any suitable database or prediction tool, including but not limited to Minimotif Miner (http: minimotifminer.org, http://mitominer.mrc-mbu.cam.ac.uk/release-4.0/embodiment.do?name=Protein % 20MTS), LocDB (see above), PTSs predictor, TargetP-2.0 (cbs.dtu.dk/services/TargetP/), ChloroP (cbs.dtu.dk/services/ChloroP/); NetNES (cbs.dtu.dk/services/NetNES/), Predotar (urgi.versailles.inra.fr/predotar/), and SignalP (cbs.dtu.dk/services/SignalP/).

### Selectable Markers and Tags

The vector and/or engineered polynucleotide of the present invention can include polynucleotide that encodes or is a selectable marker or tag, which can be a polynucleotide or polypeptide. In an embodiment, expression of the selectable markers or tags can be driven or otherwise regulated by one or more CREs of the present invention. In an embodiment, the selectable marker or tag is a polypeptide. In an embodiment, the selectable marker or tag is a polynucleotide barcode or unique molecular identifier (UMI).

It will be appreciated that In an embodiment, polynucleotide encoding such selectable markers or tags can be included in a vector and/or engineered polynucleotide of the present invention and operably coupled to one or more CREs of the present invention so as to allow for cell type, cell state, tissue type, and/or environment specific expression of the selectable marker or tag. Such techniques and methods are described elsewhere herein and will be instantly appreciated by one of ordinary skill in the art in view of this disclosure. Many such selectable markers and tags are generally known in the art and are intended to be within the scope of this disclosure.

Suitable selectable markers and tags include, but are not limited to, affinity tags, such as chitin binding protein (CBP), maltose-binding protein (MBP), glutathione-S-transferase (GST), poly(His) tag; solubilization tags such as thioredoxin (TRX) and poly (NANP), MBP, and GST; chromatography tags such as those consisting of polyanionic amino acids, such as FLAG-tag; epitope tags such as V5-tag, Myc-tag, HA-tag and NE-tag; protein tags that can allow specific enzymatic modification (such as biotinylation by biotin ligase) or chemical modification (such as reaction with FLASH-EDT2 for fluorescence imaging), DNA and/or RNA segments that contain restriction enzyme or other enzyme cleavage sites; DNA segments that encode products that provide resistance against otherwise toxic compounds including antibiotics, such as, spectinomycin, ampicillin, kanamycin, tetracycline, Basta, neomycin phosphotransferase II (NEO), hygromycin phosphotransferase (HPT) and the like; DNA and/or RNA segments that encode products that are otherwise lacking in the recipient cell (e.g., tRNA genes, auxotrophic markers); DNA and/or RNA segments that encode products which can be readily identified (e.g., phenotypic markers such as β-galactosidase, GUS; fluorescent proteins such as green fluorescent protein (GFP), cyan (CFP), yellow (YFP), red (RFP), luciferase, and cell surface proteins); polynucleotides that can generate one or more new primer sites for PCR (e.g., the juxtaposition of two DNA sequences not previously juxtaposed), DNA sequences not acted upon or acted upon by a restriction endonuclease or other DNA modifying enzyme, chemical, etc.; epitope tags (e.g. GFP, FLAG- and His-tags), and, DNA sequences that make a molecular barcode or unique molecular identifier (UMI), DNA sequences required for a specific modification (e.g., methylation) that allows its identification. Other suitable markers will be appreciated by those of skill in the art.

Selectable markers and tags can be operably linked to one or more additional gene products of the engineered polynucleotide and/or vectors described herein via suitable linkers, such as a glycine or glycine serine linkers as short as GS or GG up to (GGGGG)₃ (SEQ ID NO: 22) or (GGGGS)₃ (SEQ ID NO: 23). and other linkers described elsewhere herein.

The vector or vector system can include one or more polynucleotides encoding one or more targeting moieties. In an embodiment, the targeting moiety encoding polynucleotides can be included in the vector or vector system, such as a viral vector system, such that they are expressed within and/or on the virus particle(s) produced such that the virus particles can be targeted to specific cells, tissues, organs, etc. In an embodiment, the targeting moiety encoding polynucleotides can be included in the vector or vector system such that the gene or gene product expressed therefrom include the targeting moiety and can be targeted to specific cells, tissues, organs, etc. In an embodiment, such as non-viral carriers, the targeting moiety can be attached to the carrier (e.g., polymer, lipid, inorganic molecule, etc.) and can be capable of targeting the carrier and any attached or associated gene products from an engineered polynucleotide or vector of the present invention to specific cells, tissues, organs, etc.

### Codon Optimization of Vector Polynucleotides

As described elsewhere herein, the polynucleotide component of the engineered polynucleotide or any one or more regions of the vectors described herein can be codon optimized. In general, codon optimization refers to a process of modifying a nucleic acid sequence for enhanced expression in the host cells of interest by replacing at least one codon (e.g., about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit a particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database” available at kazusa.orjp/codon/and these tables can be adapted in a number of ways. See Nakamura, Y., et al. “Codon usage tabulated from the international DNA sequence databases: status for the year 2000” Nucl. Acids Res. 28:292 (2000). Computer algorithms for codon optimizing a particular sequence for expression in a particular host cell are also available, such as Gene Forge (Aptagen; Jacobus, PA). In an embodiment, one or more codons (e.g., 1, 2, 3, 4, 5, 10, 15, 20, 25, 50, or more, or all codons) in a sequence encoding a gene product corresponds to the most frequently used codon for a particular amino acid. As to codon usage in yeast, reference is made to the online Yeast Genome database available at yeastgenome.org/community/codon_usage.shtml, or Codon selection in yeast, Bennetzen and Hall, J Biol Chem. 1982 Mar. 25; 257 (6): 3026-31. As to codon usage in plants including algae, reference is made to Codon usage in higher plants, green algae, and cyanobacteria, Campbell and Gowri, Plant Physiol. 1990 January; 92 (1): 1-11.; as well as Codon usage in plant genes, Murray et al, Nucleic Acids Res. 1989 Jan. 25; 17 (2): 477-98; or Selection on the codon bias of chloroplast and cyanelle genes in different plant and algal lineages, Morton B R, J Mol Evol. 1998 April; 46 (4): 449-59.

The vector polynucleotide can be codon optimized for expression in a specific cell type, tissue type, organ type, and/or subject type. In an embodiment, a codon-optimized sequence is a sequence optimized for expression in a eukaryote, e.g., humans (i.e. being optimized for expression in a human or human cell), or for another eukaryote, such as another animal (e.g. a mammal or avian) as is described elsewhere herein. Such codon-optimized sequences are within the ambit of the ordinary skilled artisan in view of the description herein. In an embodiment, the polynucleotide is codon optimized for a specific cell type. Such cell types can include, but are not limited to, epithelial cells (including skin cells, cells lining the gastrointestinal tract, cells lining other hollow organs), nerve cells (nerves, brain cells, spinal column cells, nerve support cells (e.g. astrocytes, glial cells, Schwann cells etc.)), muscle cells (e.g. cardiac muscle cells, smooth muscle cells, and skeletal muscle cells), connective tissue cells (fat and other soft tissue padding cells, bone cells, tendon cells, cartilage cells), blood cells, stem cells and other progenitor cells, immune system cells, germ cells, and combinations thereof. Such codon-optimized sequences are within the ambit of the ordinary skilled artisan in view of the description herein. In an embodiment, the polynucleotide is codon optimized for a specific tissue type. Such tissue types can include, but are not limited to, muscle tissue, connective tissue, nervous tissue, and epithelial tissue. Such codon-optimized sequences are within the ambit of the ordinary skilled artisan in view of the description herein. In an embodiment, the polynucleotide is codon optimized for a specific organ. Such organs include, but are not limited to, muscles, skin, intestines, liver, spleen, brain, lungs, stomach, heart, kidneys, gallbladder, pancreas, bladder, thyroid, bone, blood vessels, blood, and combinations thereof. Such codon-optimized sequences are within the ambit of the ordinary skilled artisan in view of the description herein.

In an embodiment, a vector polynucleotide is codon optimized for expression in particular cells, such as prokaryotic or eukaryotic cells. The eukaryotic cells may be those of or derived from a particular organism, such as a plant or a mammal, including but not limited to human, or non-human eukaryote or animal or mammal as discussed herein, e.g., mouse, rat, rabbit, dog, livestock, or non-human mammal or primate.

### Vector Construction

The vectors described herein can be constructed using any suitable process or technique. In an embodiment, one or more suitable recombination and/or cloning methods or techniques can be used to design the vector(s) described herein. Suitable recombination and/or cloning techniques and/or methods can include, but not limited to, those described in U.S. Patent Publication No. US 2004/0171156 A1. Other suitable methods and techniques are described elsewhere herein.

Construction of recombinant AAV vectors is described in a number of publications, including U.S. Pat. No. 5,173,414; Tratschin et al., Mol. Cell. Biol. 5:3251-3260 (1985); Tratschin, et al., Mol. Cell. Biol. 4:2072-2081 (1984); Hermonat & Muzyczka, PNAS 81:6466-6470 (1984); and Samulski et al., J. Virol. 63:03822-3828 (1989). Any of the techniques and/or methods can be used and/or adapted for constructing an AAV or other vector described herein. nullAAV (nAAV) vectors are discussed elsewhere herein.

In an embodiment, a vector comprises one or more insertion sites, such as a restriction endonuclease recognition sequence (also referred to as a “cloning site”). In an embodiment, one or more insertion sites (e.g., about or more than about 1, 2, 3, 4, 5, 6, 7, 8, 9, 10, or more insertion sites) are located upstream and/or downstream of one or more sequence elements of one or more vectors. When multiple different guide polynucleotides are used, such in the context of a CRISPR-Cas system, a single expression construct may be used to target multiple different, corresponding target sequences within a cell. For example, a single vector may comprise about or more than about 1, 2, 3, 4, 5, 6, 7, 8, 9, 10, 15, 20, or more guide polynucleotides. In an embodiment, about or more than about 1, 2, 3, 4, 5, 6, 7, 8, 9, 10, or more guide-polynucleotide-containing vectors may be provided, and optionally delivered to a cell.

Delivery vehicles, vectors, particles, nanoparticles, formulations, and components thereof for expression of one or more elements of the engineered polynucleotides and/or vectors described herein are as used in the foregoing documents, such as International Patent Publication No. WO 2014/093622 (PCT/US2013/074667) and are discussed in greater detail herein.

### Viral Vectors

In an embodiment, the vector is a viral vector. The term of art “viral vector” and as used herein in this context refers to polynucleotide-based vectors that contain one or more elements from or based upon one or more elements of a virus that can be capable of expressing and packaging a polynucleotide, such as an engineered polynucleotide of the present invention or non-CRE component thereof, into a virus particle and producing said virus particle when used alone or with one or more other viral vectors (such as in a viral vector system). Viral vectors and systems thereof can be used for producing viral particles for delivery of and/or expression of an engineered polynucleotide of the present invention or non-CRE component thereof. The viral vector can be part of a viral vector system involving multiple vectors. In an embodiment, systems incorporating multiple viral vectors can increase the safety of these systems. Suitable viral vectors can include retroviral-based vectors, lentiviral-based vectors, adenoviral-based vectors, adeno-associated vectors, helper-dependent adenoviral (HdAd) vectors, hybrid adenoviral vectors, herpes simplex virus-based vectors, poxvirus-based vectors, and Epstein-Barr virus-based vectors. Other embodiments of viral vectors and viral particles produced therefrom are described elsewhere herein. In an embodiment, the viral vectors are configured to produce replication incompetent viral particles for improved safety of these systems.

In certain embodiments, the virus structural component, which can be encoded by one or more polynucleotides in a viral vector or vector system, comprises one or more capsid proteins including an entire capsid. In certain embodiments, such as wherein a viral capsid comprises multiple copies of different proteins, the delivery system can provide one or more of the same protein or a mixture of such proteins. For example, AAV comprises 3 capsid proteins, VP1, VP2, and VP3, thus delivery systems of the invention can comprise one or more of VP1, and/or one or more of VP2, and/or one or more of VP3. Accordingly, the present invention is applicable to a virus within the family Adenoviridae, such as Atadenovirus, e.g., Ovine atadenovirus D, Aviadenovirus, e.g., Fowl aviadenovirus A, Ichtadenovirus, e.g., Sturgeon ichtadenovirus A, Mastadenovirus (which includes adenoviruses such as all human adenoviruses), e.g., Human mastadenovirus C, and Siadenovirus, e.g., Frog siadenovirus A. Target-specific AAV capsid variants can be used or selected. Non-limiting examples include capsid variants selected to bind to chronic myelogenous leukemia cells, human CD34 PBPC cells, breast cancer cells, cells of lung, heart, dermal fibroblasts, melanoma cells, stem cells, glioblastoma cells, coronary artery endothelial cells and keratinocytes. See, e.g., Buning et al, 2015, Current Opinion in Pharmacology 24, 94-104. From teachings herein and knowledge in the art as to modifications of adenovirus (see, e.g., U.S. Pat. Nos. 9,410,129, 7,344,872, 7,256,036, 6,911,199, 6,740,525; Matthews, “Capsid-Incorporation of Antigens into Adenovirus Capsid Proteins for a Vaccine Approach,” Mol Pharm, 8 (1): 3-11 (2011)), as well as regarding modifications of AAV, the skilled person can readily obtain a modified adenovirus that has a large payload protein. Such modified adenovirus systems may be advantageous for embodiments of an engineered polynucleotide or non-CRE component thereof or gene product produced therefrom that may, when considered alone or together, be payload larger than the capacity of a native AAV. As to the viruses related to adenovirus mentioned herein, as well as to the viruses related to AAV mentioned elsewhere herein, the teachings herein as to modifying adenovirus and AAV, respectively, can be applied to those viruses without undue experimentation from this disclosure and the knowledge in the art.

In an embodiment, the viral vector is configured such that when a cargo is packaged the cargo(s) (e.g., an engineered polynucleotide or component thereof such as a non-CRE component and/or a gene product produced therefrom), is external to the capsid or virus particle. In the sense that it is not inside the capsid (enveloped or encompassed with the capsid) but is externally exposed so that it can contact the target cellular component (e.g., DNA, RNA, proteins). In an embodiment, the viral vector is configured such that all the cargo(s) are contained within the capsid after packaging.

### Split Viral Vector Systems

In an embodiment, the viral vector or vector system (be it a retroviral (e.g., AAV) or lentiviral vector) is designed so as to position the cargo(s) (e.g., an engineered polynucleotide or component thereof such as a non-CRE component and/or a gene product produced therefrom), at the internal surface of the capsid. Once formed the cargo(s) will fill most or all of the internal volume of the capsid. In other embodiments, the engineered polynucleotide of the present invention or component thereof may be modified or divided so as to occupy less of the capsid internal volume. Accordingly, in certain embodiments, the engineered polynucleotide of the present invention or component(s) thereof can be divided in two portions, one portion comprised in one viral particle or capsid and the second portion comprised in a second viral particle or capsid. In certain embodiments, by splitting the engineered polynucleotide or component thereof in two portions, space is made available to link one or more additional domains or polynucleotides to one or both of the engineered polynucleotide portions and/or gene product produced therefrom. Such systems can be referred to as “split vector systems” or in the context of the present disclosure a “split system” a “split protein” and the like. This split protein approach is also described elsewhere herein. When the concept is applied to a vector system, it thus describes putting pieces of the split proteins on different vectors thus reducing the payload of any one vector. This approach can facilitate delivery of systems where the total system size is close to or exceeds the packaging capacity of the vector. This is independent of any regulation of a gene product produced from the engineered polynucleotide or vector that can be achieved with a split system or split protein design. In certain embodiments, each part of a split-engineered gene product is attached to a member of a specific binding pair, and when bound with each other, the members of the specific binding pair maintain the parts of the engineered gene product in proximity. In certain embodiments, each part of a split-engineered gene product is associated with an inducible binding pair. An inducible binding pair is one which is capable of being switched “on” or “off” by a protein or small molecule that binds to both members of the inducible binding pair. In general, according to the invention, engineered gene product may preferably split between domains, leaving domains intact.

### Retroviral and Lentiviral Vectors

Retroviral vectors can be composed of cis-acting long terminal repeats (LTRs) with packaging capacity for up to 6-10 kb of foreign sequence. The minimum cis-acting LTRs are those sufficient for replication and packaging of the vectors, which are then used to integrate the therapeutic gene into the target cell to provide permanent transgene expression. Suitable retroviral vectors can include those based upon murine leukemia virus (MuLV), gibbon ape leukemia virus (GaLV), Simian immunodeficiency virus (SIV), human immunodeficiency virus (HIV), and combinations thereof (see, e.g., Buchscher et al., J. Virol. 66:2731-2739 (1992); Johann et al., J. Virol. 66:1635-1640 (1992); Sommnerfelt et al., Virol. 176:58-59 (1990); Wilson et al., J. Virol. 63:2374-2378 (1989); Miller et al., J. Virol. 65:2220-2224 (1991); PCT/US94/05700). The selection of a retroviral gene transfer system may therefore depend on the target tissue.

The tropism of a retrovirus can be altered by incorporating foreign envelope proteins, expanding the potential target population of target cells. Lentiviral vectors are retroviral vectors that are able to transduce or infect non-dividing cells and are described in greater detail elsewhere herein. A retrovirus can also be engineered to allow for conditional expression of the inserted transgene, such that only certain cell types are infected by the lentivirus.

Lentiviruses are complex retroviruses that have the ability to infect and express their genes in both mitotic and post-mitotic cells. Advantages of using a lentiviral approach can include the ability to transduce or infect non-dividing cells and their ability to typically produce high viral titers, which can increase efficiency or efficacy of production and delivery. Suitable lentiviral vectors include, but are not limited to, human immunodeficiency virus (HIV)-based lentiviral vectors, feline immunodeficiency virus (FIV)-based lentiviral vectors, simian immunodeficiency virus (SIV)-based lentiviral vectors, Moloney Murine Leukemia Virus (Mo-MLV), Visna-maedi virus (VMV)-based lentiviral vector, caprine arthritis-encephalitis virus (CAEV)-based lentiviral vector, bovine immune deficiency virus (BIV)-based lentiviral vector, and Equine infectious anemia (EIAV)-based lentiviral vector. In an embodiment, an HIV-based lentiviral vector system can be used. In an embodiment, an FIV-based lentiviral vector system can be used.

In an embodiment, the lentiviral vector is an EIAV-based lentiviral vector or vector system. EIAV vectors have been used to mediate expression, packaging, and/or delivery in other contexts, such as for ocular gene therapy (see, e.g., Balagaan, J Gene Med 2006; 8:275-285). In another embodiment, RetinoStat®, (see, e.g., Binley et al., HUMAN GENE THERAPY 23:980-991 (September 2012)), which describes an equine infectious anemia virus-based lentiviral gene therapy vector that expresses angiostatic proteins endostatin and angiostatin that is delivered via a subretinal injection for the treatment of the wet form of age-related macular-degeneration. Any of these vectors described in these publications can be modified for use with the present invention.

In an embodiment, the lentiviral vector or vector system thereof can be a first-generation lentiviral vector or vector system thereof. First-generation lentiviral vectors can contain a large portion of the lentivirus genome, including the gag and pol genes, other additional viral proteins (e.g. VSV-G) and other accessory genes (e.g. vif, vprm vpu, nef, and combinations thereof), regulatory genes (e.g. tat and/or rev) as well as the gene of interest between the LTRs. First-generation lentiviral vectors can result in the production of virus particles that can be capable of replication in vivo, which may not be appropriate for some instances or applications.

In an embodiment, the lentiviral vector or vector system thereof can be a second-generation lentiviral vector or vector system thereof. Second-generation lentiviral vectors do not contain one or more accessory virulence factors and do not contain all components necessary for virus particle production on the same lentiviral vector. This can result in the production of a replication-incompetent virus particle and thus increase the safety of these systems over first-generation lentiviral vectors. In an embodiment, the second-generation vector lacks one or more accessory virulence factors (e.g., vif, vprm, vpu, nef, and combinations thereof). Unlike the first-generation lentiviral vectors, no single second-generation lentiviral vector includes all features necessary to express and package a polynucleotide into a virus particle. In an embodiment, the envelope and packaging components are split between two different vectors with the gag, pol, rev, and tat genes being contained on one vector and the envelope proteins (e.g. VSV-G) are contained on a second vector. The gene of interest, its promoter, and LTRs can be included on a third vector that can be used in conjunction with the other two vectors (packaging and envelope vectors) to generate a replication-incompetent virus particle.

In an embodiment, the lentiviral vector or vector system thereof can be a third-generation lentiviral vector or vector system thereof. Third-generation lentiviral vectors and vector systems thereof have increased safety over first- and second-generation lentiviral vectors and systems thereof because, for example, the various components of the viral genome are split between two or more different vectors but used together in vitro to make virus particles, they can lack the tat gene (when a constitutively active promoter is included upstream of the LTRs), and they can include one or more deletions in 3′LTR to create self-inactivating (SIN) vectors having disrupted promoter/enhancer activity of the LTR. In an embodiment, a third-generation lentiviral vector system can include (i) a vector plasmid that contains the polynucleotide of interest and upstream promoters that are flanked by 5′ and 3′ LTRs, which can optionally include one or more deletions present in one or both of the LTRs to render the vector self-inactivating; (ii) a “packaging vector(s)” that can contain one or more genes involved in packaging a polynucleotide into a virus particle that is produced by the system (e.g. gag, pol, and rev) and upstream regulatory sequences (e.g. promoter(s)) to drive expression of the features present on the packaging vector, and (iii) an “envelope vector” that contains one or more envelope protein genes and upstream promoters. In certain embodiments, the third-generation lentiviral vector system can include at least two packaging vectors, with the gag-pol being present on a different vector than the rev gene.

In an embodiment, self-inactivating lentiviral vectors with an siRNA targeting a common exon shared by HIV tat/rev, a nucleolar-localizing TAR decoy, and an anti-CCR5-specific hammerhead ribozyme (see, e.g., DiGiusto et al. (2010) Sci Transl Med 2:36ra43) can be used with and/or adapted to the present invention.

In an embodiment, the pseudotype and infectivity or tropism of a lentivirus particle can be tuned by altering the type of envelope protein(s) included in the lentiviral vector or system thereof. As used herein, an “envelope protein” or “outer protein” means a protein exposed at the surface of a viral particle that is not a capsid protein. For example, envelope or outer proteins typically comprise proteins embedded in the envelope of the virus. In an embodiment, a lentiviral vector or vector system thereof can include a VSV-G envelope protein. VSV-G mediates viral attachment to a low-density lipoprotein (LDL) receptor (LDLR) or an LDLR family member present on a host cell, which triggers endocytosis of the viral particle by the host cell. Because LDLR is expressed by a wide variety of cells, viral particles expressing the VSV-G envelope protein can infect or transduce a wide variety of cell types. Other suitable envelope proteins can be incorporated based on the host cell that a user desires to be infected by a virus particle produced from a lentiviral vector or system thereof described herein and can include, but are not limited to, feline endogenous virus envelope protein (RD114) (see e.g., Hanawa et al. Molec. Ther. 2002 5 (3) 242-251), modified Sindbis virus envelope proteins (see e.g., Morizono et al. 2010. J. Virol. 84 (14) 6923-6934; Morizono et al. 2001. J. Virol. 75:8016-8020; Morizono et al. 2009. J. Gene Med. 11:549-558; Morizono et al. 2006 Virology 355:71-81; Morizono et al J. Gene Med. 11:655-663, Morizono et al. 2005 Nat. Med. 11:346-352), baboon retroviral envelope protein (see e.g., Girard-Gagnepain et al. 2014. Blood. 124:1221-1231); Tupaia paramyxovirus glycoproteins (see e.g., Enkirch T. et al., 2013. Gene Ther. 20:16-23); measles virus glycoproteins (see e.g., Funke et al. 2008. Molec. Ther. 16 (8): 1427-1436), rabies virus envelope proteins, MLV envelope proteins, Ebola envelope proteins, baculovirus envelope proteins, filovirus envelope proteins, hepatitis E1 and E2 envelope proteins, gp41 and gp120 of HIV, hemagglutinin, neuraminidase, M2 proteins of influenza virus, and combinations thereof.

In an embodiment, the tropism of the resulting lentiviral particle can be tuned by incorporating cell-targeting peptides into a lentiviral vector such that the cell-targeting peptides are expressed on the surface of the resulting lentiviral particle. In an embodiment, a lentiviral vector can contain an envelope protein that is fused to a cell-targeting protein (see e.g., Buchholz et al. 2015. Trends Biotechnol. 33:777-790; Bender et al. 2016. PLOS Pathog. 12(e1005461); and Friedrich et al. 2013. Mol. Ther. 2013. 21:849-859.

In an embodiment, a split-intein-mediated approach to target lentiviral particles to a specific cell type can be used (see e.g., Chamoun-Emaneulli et al. 2015. Biotechnol. Bioeng. 112:2611-2617, Ramirez et al. 2013. Protein. Eng. Des. Sel. 26:215-233). In these embodiments, a lentiviral vector can contain one-half of a splicing-deficient variant of the naturally split intein from Nostoc punctiforme fused to a cell-targeting peptide and the same or different lentiviral vector can contain the other half of the split intein fused to an envelope protein, such as a binding-deficient, fusion-competent virus envelope protein. This can result in production of a virus particle from the lentiviral vector or vector system that includes a split intein that can function as a molecular Velcro linker to link the cell-binding protein to the pseudotyped lentivirus particle. This approach can be advantageous for use where surface-incompatibilities can restrict the use of, e.g., cell-targeting peptides.

In an embodiment, a covalent-bond-forming protein-peptide pair can be incorporated into one or more of the lentiviral vectors described herein to conjugate a cell-targeting peptide to the virus particle (see e.g., Kasaraneni et al. 2018. Sci. Reports (8) No. 10990). In an embodiment, a lentiviral vector can include an N-terminal PDZ domain of InaD protein (PDZ1) and its pentapeptide ligand (TEFCA) (SEQ ID NO: 24) from NorpA, which can conjugate the cell-targeting peptide to the virus particle via a covalent bond (e.g., a disulfide bond). In an embodiment, the PDZ1 protein can be fused to an envelope protein, which can optionally be binding deficient and/or fusion competent virus envelope protein and included in a lentiviral vector. In an embodiment, the TEFCA (SEQ ID NO: 24) can be fused to a cell-targeting peptide and the TEFCA-CPT (SEQ ID NO: 24) fusion construct can be incorporated into the same or a different lentiviral vector as the PDZ1-envelope protein construct. During virus production, specific interaction between the PDZ1 and TEFCA (SEQ ID NO: 24) facilitates producing virus particles covalently functionalized with the cell targeting peptide and thus capable of targeting a specific cell-type based upon a specific interaction between the cell targeting peptide and cells expressing its binding partner. This approach can be advantageous for use where surface-incompatibilities can restrict the use of, e.g., cell-targeting peptides.

Lentiviral vectors have been disclosed as in the treatment for Parkinson's Disease, see, e.g., US Patent Publication No. 20120295960 and U.S. Pat. Nos. 7,303,910 and 7,351,585. Lentiviral vectors have also been disclosed for the treatment of ocular diseases, see e.g., US Patent Publication Nos. 20060281180, 20090007284, US20110117189; US20090017543; US20070054961, US20100317109. Lentiviral vectors have also been disclosed for delivery to the brain, see, e.g., US Patent Publication Nos. US20110293571; US20110293571, US20040013648, US20070025970, US20090111106, and U.S. Pat. No. 7,259,015. Any of these systems or a variant thereof can be used with the present invention for delivery to and/or production of a gene product in a cell.

In an embodiment, a lentiviral vector system can include one or more transfer plasmids. Transfer plasmids can be generated from various other vector backbones and can include one or more features that can work with other retroviral and/or lentiviral vectors in the system that can, for example, improve safety of the vector and/or vector system, increase virial titers, and/or increase or otherwise enhance expression of the desired insert to be expressed and/or packaged into the viral particle. Suitable features that can be included in a transfer plasmid can include, but are not limited to, 5′LTR, 3′LTR, SIN/LTR, origin of replication (Ori), selectable marker genes (e.g., antibiotic resistance genes), Psi (Ψ), RRE (rev response element), cPPT (central polypurine tract), promoters, WPRE (woodchuck hepatitis post-transcriptional regulatory element), SV40 polyadenylation signal, pUC origin, SV40 origin, F1 origin, and combinations thereof.

In another embodiment, Cocal vesiculovirus envelope pseudotyped retroviral or lentiviral vector particles are contemplated (see, e.g., US Patent Publication No. 20120164118 assigned to the Fred Hutchinson Cancer Research Center). Cocal virus is in the Vesiculovirus genus, and is a causative agent of vesicular stomatitis in mammals. Cocal virus was originally isolated from mites in Trinidad (Jonkers et al., Am. J. Vet. Res. 25:236-242 (1964)), and infections have been identified in Trinidad, Brazil, and Argentina from insects, cattle, and horses. Many of the vesiculoviruses that infect mammals have been isolated from naturally infected arthropods, suggesting that they are vector-borne. Antibodies to vesiculoviruses are common among people living in rural areas where the viruses are endemic and laboratory-acquired; infections in humans usually result in influenza-like symptoms. The Cocal virus envelope glycoprotein shares 71.5% identity at the amino acid level with VSV-G Indiana, and phylogenetic comparison of the envelope gene of vesiculoviruses shows that Cocal virus is serologically distinct from, but most closely related to, VSV-G Indiana strains among the vesiculoviruses. See e.g., Jonkers et al., Am. J. Vet. Res. 25:236-242 (1964) and Travassos da Rosa et al., Am. J. Tropical Med. & Hygiene 33:999-1006 (1984). The Cocal vesiculovirus envelope pseudotyped retroviral vector particles may include for example, lentiviral, alpharetroviral, betaretroviral, gammaretroviral, deltaretroviral, and epsilonretroviral vector particles that may comprise retroviral Gag, Pol, and/or one or more accessory protein(s) and a Cocal vesiculovirus envelope protein. In certain embodiments, the Gag, Pol, and accessory proteins are lentiviral and/or gammaretroviral. In an embodiment, a retroviral vector can contain encoding polypeptides for one or more Cocal vesiculovirus envelope proteins such that the resulting viral or pseudoviral particles are Cocal vesiculovirus envelope pseudotyped.

### Adenoviral Vectors, Helper-Dependent Adenoviral Vectors, and Hybrid Adenoviral Vectors

In an embodiment, the vector can be an adenoviral vector. In an embodiment, the adenoviral vector can include elements such that the virus particle produced using the vector or system thereof can be serotype 2 or serotype 5. In an embodiment, the polynucleotide to be delivered via the adenoviral particle can be up to about 8 kb. Thus, In an embodiment, an adenoviral vector can include a DNA polynucleotide to be delivered that can range in size from about 0.001 kb to about 8 kb. Adenoviral vectors have been used successfully in several contexts (see e.g. Teramato et al. 2000. Lancet. 355:1911-1912; Lai et al. 2002. DNA Cell. Biol. 21:895-913; Flotte et al., 1996. Hum. Gene. Ther. 7:1145-1159; and Kay et al. 2000. Nat. Genet. 24:257-261.

In an embodiment, the vector can be a helper-dependent adenoviral vector or system thereof. These are also referred to in the art as “gutless” or “gutted” vectors and are a modified generation of adenoviral vectors (see e.g. Thrasher et al. 2006. Nature. 443: E5-7). In certain embodiments of the helper-dependent adenoviral vector system one vector (the helper) can contain all the viral genes required for replication but contains a conditional gene defect in the packaging domain. The second vector of the system can contain only the ends of the viral genome, one or more engineered polynucleotides, and the native packaging recognition signal, which can allow selective packaged release from the cells (see e.g., Cideciyan et al. 2009. N Engl J Med. 361:725-727). Helper-dependent adenoviral vector systems have been successful for gene delivery in several contexts (see e.g., Simonelli et al. 2010. J Am Soc Gene Ther. 18:643-650; Cideciyan et al. 2009. N Engl J Med. 361:725-727; Crane et al. 2012. Gene Ther. 19 (4): 443-452; Alba et al. 2005. Gene Ther. 12:18-S27; Croyle et al. 2005. Gene Ther. 12:579-587; Amalfitano et al. 1998. J. Virol. 72:926-933; and Morral et al. 1999. PNAS. 96:12816-12821). The techniques and vectors described in these publications can be adapted for inclusion and delivery of the engineered polynucleotides and/or components thereof described herein. In an embodiment, the polynucleotide to be delivered via the viral particle produced from a helper-dependent adenoviral vector or system thereof can be up to about 37 kb. Thus, In an embodiment, an adenoviral vector can include a DNA polynucleotide to be delivered that can range in size from about 0.001 kb to about 37 kb (see e.g., Rosewell et al. 2011. J. Genet. Syndr. Gene Ther. Suppl. 5:001).

In an embodiment, the vector is a hybrid-adenoviral vector or system thereof. Hybrid adenoviral vectors are composed of the high transduction efficiency of a gene-deleted adenoviral vector and the long-term genome-integrating potential of adeno-associated retroviruses, lentiviruses, and transposon-based gene transfer. In an embodiment, such hybrid vector systems can result in stable transduction and limited integration sites. See e.g., Balague et al. 2000. Blood. 95:820-828; Morral et al. 1998. Hum. Gene Ther. 9:2709-2716; Kubo and Mitani. 2003. J. Virol. 77 (5): 2964-2971; Zhang et al. 2013. PloS One. 8 (10) e76771; and Cooney et al. 2015. Mol. Ther. 23 (4): 667-674), whose techniques and vectors described therein can be modified and adapted for use in the engineered polynucleotides and/or components thereof of the present invention. In an embodiment, a hybrid-adenoviral vector can include one or more features of a retrovirus and/or an adeno-associated virus. In an embodiment, the hybrid-adenoviral vector can include one or more features of a spuma retrovirus or foamy virus (FV). See e.g., Ehrhardt et al. 2007. Mol. Ther. 15:146-156 and Liu et al. 2007. Mol. Ther. 15:1834-1841, whose techniques and vectors described therein can be modified and adapted for use with the engineered polynucleotides and/or components thereof of the present invention. Advantages of using one or more features from the FVs in the hybrid-adenoviral vector or system thereof can include the ability of the viral particles produced therefrom to infect a broad range of cells, a large packaging capacity as compared to other retroviruses, and the ability to persist in quiescent (non-dividing) cells. See also e.g. Ehrhardt et al. 2007. Mol. Ther. 156:146-156 and Shuji et al. 2011. Mol. Ther. 19:76-82, whose techniques and vectors described therein can be modified and adapted for use with the engineered polynucleotides and/or components thereof of the present invention.

### Adeno Associated Viral (AAV) Vectors

In an embodiment, the vector is an adeno-associated virus (AAV) vector. See, e.g., West et al., Virology 160:38-47 (1987); U.S. Pat. No. 4,797,368; WO 93/24641; Kotin, Human Gene Therapy 5:793-801 (1994); and Muzyczka, J. Clin. Invest. 94:1351 (1994). Although similar to adenoviral vectors in some of their features, AAVs have some deficiency in their replication and/or pathogenicity and thus can be safer than adenoviral vectors. In an embodiment, the AAV can integrate into a specific site on chromosome 19 of a human cell with no observable side effects. In an embodiment, the capacity of the AAV vector, system thereof, and/or AAV particles can be up to about 4.7 kb. In an embodiment such as those where a CRISPR-Cas system is delivered as a co-therapy, utilizing homologs of the Cas effector protein that are shorter than e.g., SpCas9 (˜4104 bp) can be utilized, such as those in Table 4.

TABLE 4
Exemplary shorter Cas effector homologs.

| Species | Cas9 Size (bp) |
|---|---|
| Corynebacterium diphtheriae | 3252 |
| Eubacterium ventriosum | 3321 |
| Streptococcus pasteurianus | 3390 |
| Lactobacillus farciminis | 3378 |
| Sphaerochaeta globus | 3537 |
| Azospirillum B510 | 3504 |
| Gluconacetobacter diazotrophicus | 3150 |
| Neisseria cinerea | 3246 |
| Roseburia intestinalis | 3420 |
| Parvibaculum lavamentivorans | 3111 |
| Staphylococcus aureus | 3159 |
| Nitratifractor salsuginis DSM 16511 | 3396 |
| Campylobacter lari CF89-12 | 3009 |
| Campylobacter jejuni | 2952 |
| Streptococcus thermophilus LMD-9 | 3396 |

The AAV vector or system thereof can include one or more regulatory molecules. In an embodiment, the regulatory molecules can be promoters, enhancers, repressors, and the like, which are described in greater detail elsewhere herein. In an embodiment, the AAV vector or system thereof can include one or more polynucleotides that can encode one or more regulatory proteins. In an embodiment, the one or more regulatory proteins can be selected from Rep78, Rep68, Rep52, Rep40, variants thereof, and combinations thereof.

The AAV vector or system thereof can include one or more polynucleotides that can encode one or more capsid proteins. The capsid proteins can be selected from VP1, VP2, VP3, and combinations thereof. The capsid proteins can be capable of assembling into a protein shell of the AAV virus particle. In an embodiment, the AAV capsid can contain 60 capsid proteins. In an embodiment, the ratio of VP1:VP2:VP3 in a capsid can be about 1:1:10.

In an embodiment, the AAV vector or system thereof can include one or more adenovirus helper factors or polynucleotides that can encode one or more adenovirus helper factors. Such adenovirus helper factors can include, but are not limited to, E1A, E1B, E2A, E4ORF6, and VA RNAs. In an embodiment, a producing host cell line expresses one or more of the adenovirus helper factors.

The AAV vector or system thereof can be configured to produce AAV particles having a specific serotype. In an embodiment, the serotype can be AAV-1, AAV-2, AAV-3, AAV-4, AAV-5, AAV-6, AAV-7, AAV-8, AAV-9 or any combinations thereof. In an embodiment, the AAV can be AAV-1, AAV-2, AAV-5 or any combination thereof. One can select the AAV serotype of the AAV with regard to the cells to be targeted; e.g., one can select AAV serotypes 1, 2, 5 or a hybrid capsid AAV-1, AAV-2, AAV-5 or any combination thereof for targeting brain and/or neuronal cells; and one can select AAV-4 for targeting cardiac tissue; and one can select AAV-8 for delivery to the liver. Thus, In an embodiment, an AAV vector or system thereof capable of producing AAV particles capable of targeting the brain and/or neuronal cells can be configured to generate AAV particles having serotypes 1, 2, 5 or a hybrid capsid AAV-1, AAV-2, AAV-5 or any combination thereof. In an embodiment, an AAV vector or system thereof capable of producing AAV particles capable of targeting cardiac tissue can be configured to generate an AAV particle having an AAV-4 serotype. In an embodiment, an AAV vector or system thereof capable of producing AAV particles capable of targeting the liver can be configured to generate an AAV having an AAV-8 serotype. In an embodiment, the AAV vector is a hybrid AAV vector or system thereof. Hybrid AAVs are AAVs that include genomes with elements from one serotype that are packaged into a capsid derived from at least one different serotype. For example, if it is the recombinant AAV2/5 (rAAV2/5) that is to be produced, and if the production method is based on the helper-free, transient transfection method discussed elsewhere herein, all plasmids but the RepCap (pRepCap) plasmid will be the same. In the RepCap plasmid, called pRep2/Cap5, the Rep gene is still derived from AAV-2, while the Cap gene is derived from AAV-5. The production scheme is the same as the above-mentioned approach for AAV-2 production. The resulting rAAV is called rAAV2/5, in which the genome is based on recombinant AAV-2, while the capsid is based on AAV-5. It is assumed the cell or tissue-tropism displayed by this AAV2/5 hybrid virus should be the same as that of AAV-5. This can be applied to generate other hybrid serotypes.

A tabulation of certain AAV serotypes as to these cells can be found in Grimm, D. et al, J. Virol. 82:5887-5911 (2008) at Table 3.

In an embodiment, the AAV vector or system thereof is configured as a “gutless” vector, similar to that described in connection with a retroviral vector. In an embodiment, the “gutless” AAV vector or system thereof can have the cis-acting viral DNA elements involved in genome amplification and packaging in linkage with the heterologous sequences of interest (e.g., an engineered polynucleotide of the present invention or component thereof)

In an embodiment, the AAV vectors are produced in insect cells, e.g., Spodoptera frugiperda Sf9 insect cells, grown in serum-free suspension culture. Serum-free insect cells can be purchased from commercial vendors, e.g., Sigma Aldrich (EX-CELL 405).

In an embodiment, an AAV vector or vector system can contain or consists essentially of one or more polynucleotides encoding one or more components of a CRISPR system. In an embodiment, the AAV vector or vector system can contain a plurality of cassettes comprising or consisting a first cassette comprising or consisting essentially of a promoter, a nucleic acid molecule encoding a CRISPR-associated (Cas) protein (putative nuclease or helicase proteins), e.g., a Cas protein and a terminator, and a two, or more, advantageously up to the packaging size limit of the vector, e.g., in total (including the first cassette) five, cassettes comprising or consisting essentially of a promoter, nucleic acid molecule encoding guide RNA (gRNA) and a terminator (e.g., each cassette schematically represented as Promoter-gRNA1-terminator, Promoter-gRNA2-terminator, . . . . Promoter-gRNA (N)-terminator; where N is a number that can be inserted that is at an upper limit of the packaging size limit of the vector), or two or more individual rAAVs, each containing one or more than one cassette of a CRISPR system, e.g., a first rAAV containing the first cassette comprising or consisting essentially of a promoter, a nucleic acid molecule encoding Cas, e.g., a Cas and a terminator, and a second rAAV containing a plurality of cassettes comprising or consisting essentially of a promoter, nucleic acid molecule encoding guide RNA (gRNA) and a terminator (e.g., each cassette schematically represented as Promoter-gRNA1-terminator, Promoter-gRNA2-terminator, . . . . Promoter-gRNA (N)-terminator; where N is a number that can be inserted that is at an upper limit of the packaging size limit of the vector). As rAAV is a DNA virus, the nucleic acid molecules in the herein discussion concerning AAV or rAAV are advantageously DNA. In an embodiment, the promoter or other regulatory element is a CRE of the present invention or another tissue-specific promoter or another tissue-specific regulatory element. Suitable tissue-specific regulatory elements, including promoters, are described in greater detail elsewhere herein.

In another embodiment, the invention provides a non-naturally occurring or engineered polynucleotide or component thereof or gene product therefrom, optionally CRISPR-Cas system protein or polynucleotide associated with Adeno Associated Virus (AAV), e.g., an AAV comprising a CRISPR-Cas system protein or polynucleotide as a fusion, with or without a linker, to or with an AAV capsid protein such as VP1, VP2, and/or VP3. Incorporation of proteins in viral capsids is described in e.g., Rybniker et al., “Incorporation of Antigens into Viral Capsids Augments Immunogenicity of Adeno-Associated Virus Vector-Based Vaccines,” J Virol. December 2012; 86 (24): 13800-13804, Lux K, et al. 2005; Green fluorescent protein-tagged adeno-associated virus particles allow the study of cytosolic and nuclear trafficking. J. Virol. 79:11776-11787; Munch R C, et al. 2012. “Displaying high-affinity ligands on adeno-associated viral vectors enables tumor cell-specific and safe gene transfer.” Mol. Ther. [doi: 10.1038/mt.2012.186 and Warrington K H, Jr, et al. 2004. Adeno-associated virus type 2 VP2 capsid protein is nonessential and can tolerate large peptide insertions at its N terminus. J. Virol. 78:6595-6609, which can each be adapted for use with the present invention. It will be understood by those skilled in the art that the modifications described herein, if inserted into the AAV capsid gene (cap gene), may result in modifications in the VP1, VP2 and/or VP3 capsid subunits. Alternatively, the capsid subunits can be expressed independently to achieve modification in only one or two of the capsid subunits (VP1, VP2, VP3, VP1+VP2, VP1+VP3, or VP2+VP3). One can modify the cap gene to have expressed at a desired location a non-capsid protein, advantageously a large payload protein, such as a CRISPR-protein or other gene product. Likewise, these can be fusions, with the protein, e.g., a large payload protein such as a CRISPR-protein fused in a manner analogous to prior art fusions. See, e.g., US Patent Publication 20090215879; Nance et al., “Perspective on Adeno-Associated Virus Capsid Modification for Duchenne Muscular Dystrophy Gene Therapy,” Hum Gene Ther. 26 (12): 786-800 (2015) and documents cited therein, incorporated herein by reference. The skilled person, from this disclosure and the knowledge in the art can make and use modified AAV or AAV capsid as in the herein invention, and through this disclosure, one knows now that large payload proteins can be fused to the AAV capsid. In an embodiment, the AAV-capsid recombinant AAVs contain proteins and/or nucleic acid molecule(s) encoding or providing a CRISPR-Cas system or other gene product to a cell. In an embodiment, the CRISPR-Cas system or the gene product is assembled from the nucleic acid molecule(s) contained in the AAV and a protein component on a surface of the capsid, such as outer or inner surface. The instant invention is also applicable to a virus in the genus Dependoparvovirus or in the family Parvoviridae, for instance, AAV, or a virus of Amdoparvovirus, e.g., Carnivore amdoparvovirus 1, a virus of Aveparvovirus, e.g., Galliform aveparvovirus 1, a virus of Bocaparvovirus, e.g., Ungulate bocaparvovirus 1, a virus of Copiparvovirus, e.g., Ungulate copiparvovirus 1, a virus of Dependoparvovirus, e.g., Adeno-associated dependoparvovirus A, a virus of Erythroparvovirus, e.g., Primate erythroparvovirus 1, a virus of Protoparvovirus, e.g., Rodent protoparvovirus 1, a virus of Tetraparvovirus, e.g., Primate tetraparvovirus 1. Thus, a virus within the family Parvoviridae or the genus Dependoparvovirus or any of the other foregoing genera within Parvoviridae is contemplated as within the invention with discussion herein as to AAV applicable to such other viruses.

In an embodiment, a CRISPR-Cas system or component thereof or other gene product is external to the capsid or virus particle in the sense that it is not inside the capsid (enveloped or encompassed with the capsid), but is externally exposed so that it can contact the target cellular component (e.g., DNA, RNA, and/or protein). In an embodiment, a CRISPR-Cas system or component thereof or other gene product is associated with the AAV VP2 domain by way of a fusion protein. In an embodiment, the association may be considered to be a modification of the VP2 domain. In an embodiment, the AAV VP2 domain may be associated (or tethered) to a CRISPR-Cas system or component thereof or other gene product via a connector protein, for example using a system such as the streptavidin-biotin system. In an embodiment, the CRISPR-Cas system or component thereof or another gene product and associated AAV VP2 domain are encoded by a polynucleotide. In one embodiment, the invention provides a non-naturally occurring modified AAV having a VP2-CRISPR-Cas system or component thereof or another gene product capsid protein, wherein the CRISPR-Cas system or component thereof or another gene product is part of or tethered to the VP2 domain. In an embodiment, the CRISPR-Cas system or component thereof or another gene product is fused to the VP2 domain to produce a modified AAV having a VP2-CRISPR-CRISPR-Cas system or component thereof or another gene product fusion capsid protein. In an embodiment, the VP2-CRISPR-Cas system or component thereof or another gene product capsid protein further comprises a linker, whereby the VP2-CRISPR-Cas system or component thereof or another gene product is distanced from the remainder of the AAV. In an embodiment, the VP2-CRISPR-Cas system or component thereof or another gene product capsid protein further comprises at least one protein complex, e.g., CRISPR complex, such as a CRISPR-Cas complex guide RNA that targets a particular cellular polynucleotide target (e.g., a DNA or an RNA molecule).

In one embodiment, the invention provides a non-naturally occurring or engineered composition comprising a CRISPR-Cas system or component thereof or other gene product. In some of such embodiments, the CRISPR-Cas system or component thereof or other gene product is part of or tethered to an AAV capsid domain, i.e., VP1, VP2, or VP3 domain of Adeno-Associated Virus (AAV) capsid. In an embodiment, part of a CRISPR-Cas system or component thereof or other gene product tethered to an AAV capsid domain is associated with an AAV capsid domain. In an embodiment, a CRISPR-Cas system or component thereof or other gene product may be fused to the AAV capsid domain. In an embodiment, the fusion may be to the N-terminal end of the AAV capsid domain. As such, In an embodiment, the CRISPR-Cas system or component thereof or other gene product is fused to the N-terminal end of the AAV capsid domain. In an embodiment, an NLS and/or a linker (such as a GlySer linker) may be positioned between the C-terminal end of the CRISPR-Cas system or component thereof or other gene product and the N-terminal end of the AAV capsid domain. In an embodiment, the fusion may be to the C-terminal end of the AAV capsid domain. In an embodiment, this is not preferred due to the fact that the VP1, VP2, and VP3 domains of AAV are alternative splices of the same RNA and so a C-terminal fusion may affect all three domains. In an embodiment, the AAV capsid domain is truncated. In an embodiment, some or all of the AAV capsid domain is removed. In an embodiment, some of the AAV capsid domain is removed and replaced with a linker (such as a GlySer linker), typically leaving the N-terminal and C-terminal ends of the AAV capsid domain intact, such as the first 2, 5, or 10 amino acids. In this way, the internal (non-terminal) portion of the VP3 domain may be replaced with a linker. It some embodiments, the linker is fused to the CRISPR-Cas system or component thereof or other gene product. A branched linker may be used. In such embodiments, a CRISPR-Cas system or component thereof or other gene product is fused to the end of one of the branches. Without being bound by theory, this allows for some degree of spatial separation between the capsid and the CRISPR-Cas system or component thereof or other gene product. In this way, the CRISPR-Cas system or component thereof or other gene product is part of (or fused to) the AAV capsid domain.

In other embodiments, the CRISPR-Cas system or component thereof or other gene product may be fused in frame within, e.g., internal to, the AAV capsid domain. Thus, In an embodiment, the AAV capsid domain again preferably retains its N-terminal and C-terminal ends. In this case, a linker is preferred, In an embodiment, either at one or both ends of the CRISPR-Cas system or component thereof or other gene product. In this way, the CRISPR-Cas system or component thereof or other gene product is again part of (or fused to) the AAV capsid domain. In certain embodiments, the positioning of the CRISPR enzyme is such that the CRISPR-Cas system or component thereof or other gene product is at the external surface of the viral capsid once formed. In one embodiment, the invention provides a non-naturally occurring or engineered composition comprising a CRISPR-Cas system or component thereof or other gene product or other gene product associated with an AAV capsid domain of the AAV capsid. In this context, “associated” refers, In an embodiment to fused, or In an embodiment bound to, or In an embodiment tethered to. The CRISPR-Cas system or component thereof or other gene product may, In an embodiment, be tethered to the VP1, VP2, or VP3 domain. This may be via a connector protein or tethering system such as the biotin-streptavidin system. In one example, a biotinylation sequence (15 amino acids) could therefore be fused to a CRISPR-Cas system or component thereof or other gene product. When a fusion of the AAV capsid domain, especially the N-terminus of the AAV capsid domain, with streptavidin is also provided, the two will therefore associate with very high affinity. Thus, In an embodiment, provided is a composition or system comprising an engineered CRISPR-Cas system or component thereof or other gene product-biotin fusion and a streptavidin-AAV capsid domain arrangement, such as a fusion. The CRISPR-Cas system or component thereof or other gene product-biotin and streptavidin-AAV capsid domain forms a single complex when the two parts are brought together. NLSs may also be incorporated between the CRISPR-Cas system or component thereof or other gene product and the biotin; and/or between the streptavidin and the AAV capsid domain.

As such, provided is a fusion of a CRISPR-Cas system or component thereof or other gene product with a connector protein specific for a high-affinity ligand for that connector, whereas the AAV VP2 domain is bound to said high-affinity ligand. For example, streptavidin may be the connector fused to the CRISPR-Cas system or component thereof or other gene product, while biotin may be bound to the AAV VP2 domain. Upon co-localization, the streptavidin will bind to the biotin, thus connecting the CRISPR-Cas system or component thereof or other gene product to the AAV VP2 domain. The reverse arrangement is also possible. In an embodiment, a biotinylation sequence (15 amino acids) could therefore be fused to the AAV VP2 domain, especially the N-terminus of the AAV VP2 domain. A fusion of a CRISPR-Cas system or component thereof or other gene product with streptavidin is also preferred, In an embodiment. In an embodiment, the biotinylated AAV capsids with streptavidin-CRISPR-Cas system or component thereof or other gene product(s) are assembled in vitro. This way the AAV capsids should assemble in a straightforward manner and CRISPR-Cas system or component thereof or other gene product-streptavidin fusion can be added after assembly of the capsid. In other embodiments a biotinylation sequence (15 amino acids) could therefore be fused to the CRISPR-Cas system or component thereof or other gene product, together with a fusion of the AAV VP2 domain, especially the N-terminus of the AAV VP2 domain, with streptavidin. For simplicity, a fusion of the CRISPR-Cas system or component thereof or other gene product and the AAV VP2 domain is preferred In an embodiment. In an embodiment, the fusion may be to the N-terminal end of the CRISPR-Cas system or component thereof or other gene product. In other words, In an embodiment, the AAV and the CRISPR-Cas system or component thereof or other gene product are associated via fusion. In an embodiment, the AAV and CRISPR-Cas system or component thereof or other gene product are associated via fusion including a linker. Suitable linkers are discussed herein but include Gly Ser linkers. Fusion to the N-terminus of AAV VP2 domain is preferred, In an embodiment. In an embodiment, a CRISPR-Cas system or component thereof or other gene product comprises at least one Nuclear Localization Signal (NLS). In a further embodiment, the present invention provides compositions comprising the CRISPR-Cas system or component thereof or other gene product and associated AAV VP2 domain or the polynucleotides or vectors described herein. Such compositions and formulations are discussed elsewhere herein.

An alternative tether may be to fuse or otherwise associate the AAV capsid domain to an adaptor protein which binds to or recognizes to a corresponding RNA sequence or motif. In an embodiment, the adaptor is or comprises a binding protein which recognizes and binds (or is bound by) an RNA sequence specific for said binding protein. In an embodiment, a preferred example is the MS2 (see Konermann et al. Nature 517 (7536): 583-588 (2015), cited infra, incorporated herein by reference) binding protein which recognizes and binds (or is bound by) an RNA sequence specific for the MS2 protein. In an embodiment, the RNA sequence specific for a binding protein is a gRNA that can bind a Cas protein.

With the AAV capsid domain associated with the adaptor protein, a CRISPR-Cas system or component thereof or other gene product may, In an embodiment, be tethered to the adaptor protein of the AAV capsid domain. The CRISPR-Cas system or component thereof or other gene product may, In an embodiment, be tethered to the adaptor protein of the AAV capsid domain via the CRISPR-Cas system or component thereof or other gene product being in a complex with a modified guide, see Konermann et al. Id. The modified guide is, In an embodiment, an sgRNA. In an embodiment, the modified guide comprises a distinct RNA sequence; see, e.g., International Patent Application No. PCT/US14/70175, incorporated herein by reference. In an embodiment, the distinct RNA sequence is an aptamer. Thus, corresponding aptamer-adaptor protein systems are preferred. One or more functional domains may also be associated with the adaptor protein. An example of a preferred arrangement would be: [AAV capsid domain-adaptor protein]-[modified guide-CRISPR-Cas system or component thereof or other gene product].

In certain embodiments, the positioning of the CRISPR-Cas system or component thereof or other gene product is such that the CRISPR-Cas system or component thereof or other gene product is at the internal surface of the viral capsid once formed. In one embodiment, the invention provides a non-naturally occurring or engineered composition comprising a CRISPR-Cas system or component thereof or other gene product associated with an internal surface of an AAV capsid domain. Here again, associated may mean In an embodiment fused, or In an embodiment bound to, or In an embodiment tethered to. The CRISPR-Cas system or component thereof or other gene product may, In an embodiment, be tethered to the VP1, VP2, or VP3 domain such that it locates to the internal surface of the viral capsid once formed. This may be via a connector protein or tethering system such as the biotin-streptavidin system as described above and/or elsewhere herein.

In one embodiment, a co-therapy can include a non-naturally occurring CRISPR-Cas system comprising an AAV-Cas protein and a guide RNA that targets a DNA molecule encoding a gene product in a cell, whereby the guide RNA targets the DNA molecule encoding the gene product and the Cas protein cleaves the DNA molecule encoding the gene product, whereby expression of the gene product is altered; and, wherein the Cas protein and the guide RNA do not naturally occur together. The invention comprehends the guide RNA comprising a guide sequence fused to a Trans-activating CRISPR (tracr) sequence. In a preferred embodiment, the Cas protein is a Cas9, a Cas13, or a Cas 12 protein. Other suitable Cas proteins are described elsewhere herein. In an embodiment, the polynucleotide encoding the Cas protein is codon optimized for expression in a eukaryotic cell. In an embodiment, the eukaryotic cell is a mammalian cell and in a more preferred embodiment the mammalian cell is a human cell. In a further embodiment, the expression of the gene product is decreased.

In another embodiment, a co-therapy comprises non-naturally occurring vector system comprising one or more vectors comprising a first regulatory element operably linked to a CRISPR-Cas system guide RNA that targets a DNA molecule encoding a gene product and an AAV-Cas protein. The components may be located on same or different vectors of the system, or may be the same vector whereby the AAV-Cas protein also delivers the RNA of the CRISPR system. The guide RNA targets the DNA molecule encoding the gene product in a cell and the AAV-Cas protein may cleave the DNA molecule encoding the gene product (it may cleave one or both strands or have substantially no nuclease activity), whereby expression of the gene product is altered; and, wherein the AAV-Cas protein and the guide RNA do not naturally occur together. The invention comprehends the guide RNA comprising a guide sequence fused to a tracr sequence. In an embodiment of the invention, the AAV-Cas protein is a type II AAV-CRISPR-Cas protein and in a preferred embodiment the AAV-Cas protein is an AAV-Cas9, AAV-Cas12, or AAV-Cas13 protein. The invention further comprehends the coding for the AAV-Cas protein being codon optimized for expression in a eukaryotic cell. In a preferred embodiment, the eukaryotic cell is a mammalian cell and in a more preferred embodiment, the mammalian cell is a human cell. In a further embodiment of the invention, the expression of the gene product is decreased.

In one embodiment, the invention provides a vector system comprising one or more vectors. In an embodiment, the system comprises a CRISPR-Cas co-therapy that comprises: (a) a first regulatory element operably linked to a tracr mate sequence and one or more insertion sites for inserting one or more guide sequences upstream of the tracr mate sequence, wherein when expressed, the guide sequence directs sequence-specific binding of an AAV-CRISPR complex to a target sequence in a eukaryotic cell, wherein the CRISPR complex comprises a AAV-CRISPR enzyme complexed with (1) the guide sequence that is hybridized to the target sequence, and (2) the tracr mate sequence that is hybridized to the tracr sequence; and (b) said AAV-CRISPR enzyme comprising at least one nuclear localization sequence and/or at least one nuclear export signal (NES); wherein components (a) and (b) are located on or in the same or different vectors of the system. In an embodiment, component (a) further comprises the tracr sequence downstream of the tracr mate sequence under the control of the first regulatory element. In an embodiment, component (a) further comprises two or more guide sequences operably linked to the first regulatory element, wherein when expressed, each of the two or more guide sequences direct sequence-specific binding of an AAV-CRISPR complex to a different target sequence in a eukaryotic cell. In an embodiment, the system comprises the tracr sequence under the control of a third regulatory element, such as a polymerase III promoter. In an embodiment, the tracr sequence exhibits at least 50%, 60%, 70%, 80%, 90%, 95%, or 99% of sequence complementarity along the length of the tracr mate sequence when optimally aligned. Determining optimal alignment is within the purview of one of skill in the art. For example, there are publicly and commercially available alignment algorithms and programs such as, but not limited to, ClustalW, Smith-Waterman in matlab, Bowtie, Geneious, Biopython and SeqMan. In an embodiment, the AAV-CRISPR complex comprises one or more nuclear localization sequences of sufficient strength to drive accumulation of said CRISPR complex in a detectable amount in the nucleus of a eukaryotic cell. Without wishing to be bound by theory, it is believed that a nuclear localization sequence is not necessary for AAV-CRISPR complex activity in eukaryotes, but that including such sequences enhances activity of the system, especially as to targeting nucleic acid molecules in the nucleus and/or having molecules exit the nucleus. In an embodiment, the AAV-CRISPR enzyme is an AAV-Cas enzyme. In an embodiment, the AAV-Cas enzyme is derived from S. pneumoniae, S. pyogenes, S. thermophiles, F. novicida or S. aureus Cas9, Cas12 (e.g., Cas12a), Cas13, etc. (e.g., a Cas protein of one of these organisms modified to have or be associated with at least one AAV) and may include further mutations or alterations or be a chimeric Cas9. The enzyme may be an AAV-Cas9 homolog or ortholog. In an embodiment, the AAV-CRISPR enzyme is codon-optimized for expression in a eukaryotic cell. In an embodiment, the AAV-CRISPR enzyme directs cleavage of one or two strands at the location of the target sequence. In an embodiment, the AAV-CRISPR enzyme lacks DNA strand cleavage activity. In an embodiment, the first regulatory element is a polymerase III promoter. In an embodiment, the second regulatory element is a polymerase II promoter. In an embodiment, the guide sequence is at least 15, 16, 17, 18, 19, 20, 25 nucleotides, or between 10-30, or between 15-25, or between 15-20 nucleotides in length.

In general, In an embodiment, the AAV further comprises a repair template. It will be appreciated that comprises in the phrase “the virus comprises . . . ”, “the AAV comprises . . . ”, “the lentiviral vector LVVO”, “the LVV comprises”, and/or the like may mean encompassed within the viral capsid or that the virus encodes the comprised protein or polynucleotide such as a repair template, gRNA, mRNA, and/or the like. In an embodiment, one or more, preferably two or more guide RNAs, may be comprised/encompassed within the AAV vector. Two may be preferred, In an embodiment, as it allows for multiplexing or dual nickase approaches. Particularly for multiplexing, two or more guides may be used. In fact, In an embodiment, three or more, four or more, five or more, or even six or more guide RNAs may be comprised/encompassed within the AAV. More space has been freed up within the AAV by virtue of the fact that the AAV no longer needs to comprise/encompass the CRISPR enzyme. In each of these instances, a repair template may also be provided comprised/encompassed within the AAV. In an embodiment, the repair template corresponds to or includes the DNA target.

### Herpes Simplex Viral Vectors

In an embodiment, the vector can be a Herpes Simplex Viral (HSV)-based vector or system thereof. HSV systems can include the disabled infections single copy (DISC) viruses, which are composed of a glycoprotein H defective mutant HSV genome. When the defective HSV is propagated in complementing cells, virus particles can be generated that are capable of infecting subsequent cells, permanently replicating their own genome but are not capable of producing more infectious particles. See e.g., 2009. Trobridge. Exp. Opin. Biol. Ther. 9:1427-1436, whose techniques and vectors described therein can be modified and adapted for use in the with the present invention. In an embodiment where an HSV vector or system thereof is utilized, the host cell can be a complementing cell. In an embodiment, the HSV vector or system thereof can be capable of producing virus particles capable of delivering a polynucleotide cargo of up to 150 kb. Thus, In an embodiment, the CRISPR-Cas system or component thereof or other gene product or encoding polynucleotide(s) included in the HSV-based viral vector or system thereof can sum from about 0.001 to about 150 kb. HSV-based vectors and systems thereof have been successfully used in several contexts including various models of neurologic disorders. See e.g., Cockrell et al. 2007. Mol. Biotechnol. 36:184-204; Kafri T. 2004. Mol. Biol. 246:367-390; Balaggan and Ali. 2012. Gene Ther. 19:145-153; Wong et al. 2006. Hum. Gen. Ther. 2002. 17:1-9; Azzouz et al. J. Neruosci. 22L10302-10312; and Betchen and Kaplitt. 2003. Curr. Opin. Neurol. 16:487-493, whose techniques and vectors described therein can be modified and adapted for use in the engineered Acr delivery system and/or CRISPR-Cas co-therapy.

### Poxvirus Vectors

In an embodiment, the vector can be a poxvirus vector or system thereof. In an embodiment, the poxvirus vector can result in cytoplasmic expression of one or more engineered Acr delivery system and/or CRISPR-Cas co-therapy polynucleotides described herein. In an embodiment, the capacity of a poxvirus vector or system thereof can be about 25 kb or more. In an embodiment, a poxvirus vector or system thereof can include one or more CRISPR-Cas system polynucleotides described herein.

### Viral Vectors for Delivery to Plants

The systems and compositions may be delivered to plant cells using viral vehicles. In particular embodiments, the compositions and systems may be introduced in the plant cells using a plant viral vector (e.g., as described in Scholthof et al. 1996, Annu Rev Phytopathol. 1996; 34:299-323). Such viral vector may be a vector from a DNA virus, e.g., geminivirus (e.g., cabbage leaf curl virus, bean yellow dwarf virus, wheat dwarf virus, tomato leaf curl virus, maize streak virus, tobacco leaf curl virus, or tomato golden mosaic virus) or nanovirus (e.g., Faba bean necrotic yellow virus). The viral vector may be a vector from an RNA virus, e.g., tobravirus (e.g., tobacco rattle virus, tobacco mosaic virus), potexvirus (e.g., potato virus X), or hordeivirus (e.g., barley stripe mosaic virus). The replicating genomes of plant viruses may be non-integrative vectors.

### Virus-Like Particles and Vectors

In an embodiment, the vector is a vector that is capable of generating virus-like particles (VLPs). VLPs is a term of art that refers to particles produced from virus proteins, such as capsid or other proteins, but that do not contain the native viral genetic materials. Exemplary VLPs and their production systems and vectors for delivery of an engineered Acr delivery system described herein are described in e.g., Bhat et al., Viruses 14 (2): 383 (2022) doi: 10.3390/v14020383; Hill et al., Curr Protein Pept Sci. (2018) 19 (1): 112-127; Schwarz B et al., Adv Virus Res. 2017. 97:1-60 doi: 10.1016/bs.aivir.2016.09.002; Banskota et al., Cell. 2022. 185 (2): 250-265; Ikwuagwu and Tullman-Ercek. Curr Opin Biotechnol. 2022. 78:102785 doi: 10.1016/j.copbio.2022.102785; Zdanowicz and Chroboczek. Acta Biochim Pol. 2016: 63 (3): 469-473; Suffian and Al-Jamal et al., Adv. Drug Deliv. Rev. 2022. 180:114030 doi: 10.1016/j.addr.2021.114030; and Segel et al., Science. 373:6557 (2021).

Virus Particle Production from Viral Vectors

### Retroviral Production

In an embodiment, one or more viral vectors and/or systems thereof can be delivered to a suitable cell line for production of virus particles containing the polynucleotide or other payload to be delivered to a host cell. Suitable host cells for virus production from viral vectors and systems thereof described herein are known in the art and are commercially available. For example, suitable host cells include HEK 293 cells and its variants (HEK 293T and HEK 293TN cells). In an embodiment, the suitable host cell for virus production from viral vectors and systems thereof described herein can stably express one or more genes involved in packaging (e.g. pol, gag, and/or VSV-G) and/or other supporting genes.

In an embodiment, after delivery of one or more viral vectors to the suitable host cells for virus production from viral vectors and systems thereof, the cells are incubated for an appropriate length of time to allow for viral gene expression from the vectors, packaging of the polynucleotide to be delivered (e.g., an invention engineered Acr delivery system and/or CRISPR-Cas co-therapy polynucleotide), and virus particle assembly, and secretion of mature virus particles into the culture media. Various other methods and techniques are generally known to those of ordinary skill in the art.

Mature virus particles can be collected from the culture media by a suitable method. In an embodiment, this can involve centrifugation to concentrate the virus. The titer of the composition containing the collected virus particles can be obtained using a suitable method. Such methods can include transducing a suitable cell line (e.g. NIH 3T3 cells) and determining transduction efficiency and infectivity in that cell line by a suitable method. Suitable methods include PCR-based methods, flow cytometry, and antibiotic selection-based methods. Various other methods and techniques are generally known to those of ordinary skill in the art. The concentration of virus particles can be adjusted as needed. In an embodiment, the resulting composition containing virus particles can contain 1×10¹-1×10²⁰ particles/mL.

Lentiviruses may be prepared from any lentiviral vector or vector system described herein. In one example embodiment, after cloning pCasES10 (which contains a lentiviral transfer plasmid backbone), HEK293FT at low passage (p=5) can be seeded in a T-75 flask to 50% confluence the day before transfection in DMEM with 10% fetal bovine serum and without antibiotics. After 20 hours, the media can be changed to OptiMEM (serum-free) media and transfection of the lentiviral vectors can be done 4 hours later. Cells can be transfected with 10 μg of lentiviral transfer plasmid (pCasES10) and the appropriate packaging plasmids (e.g., 5 μg of pMD2.G (VSV-g pseudotype), and 7.5ug of psPAX2 (gag/pol/rev/tat)). Transfection can be carried out in 4 mL OptiMEM with a cationic lipid delivery agent (50 μL Lipofectamine 2000 and 100 μl Plus reagent). After 6 hours, the media can be changed to antibiotic-free DMEM with 10% fetal bovine serum. These methods can use serum during cell culture, but serum-free methods are preferred.

Following transfection and allowing the producing cells (also referred to as packaging cells) to package and produce virus particles with packaged cargo, the lentiviral particles can be purified. In an exemplary embodiment, virus-containing supernatants can be harvested after 48 hours. Collected virus-containing supernatants can first be cleared of debris and filtered through a 0.45 μm low protein binding (PVDF) filter. They can then be spun in an ultracentrifuge for 2 hours at 24,000 rpm. The resulting virus-containing pellets can be resuspended in 50 μl of DMEM overnight at 4 degrees C. They can be then aliquoted and used immediately or immediately frozen at-80 degrees C. for storage.

### AAV Particle Production

There are two main strategies for producing AAV particles from AAV vectors and systems thereof, such as those described herein, which depend on how the adenovirus helper factors are provided (helper-v. helper-free). In an embodiment, a method of producing AAV particles from AAV vectors and systems thereof can include adenovirus infection into cell lines that stably harbor AAV replication and capsid encoding polynucleotides along with AAV vector containing the polynucleotide to be packaged and delivered by the resulting AAV particle (e.g. the engineered Acr delivery system and/or CRISPR-Cas system polynucleotide(s)). In an embodiment, a method of producing AAV particles from AAV vectors and systems thereof can be a “helper-free” method, which includes co-transfection of an appropriate producing cell line with three vectors (e.g. plasmid vectors): (1) an AAV vector that contains a polynucleotide of interest (e.g. the engineered Acr delivery system and/or CRISPR-Cas system polynucleotide(s)) between 2 ITRs; (2) a vector that carries the AAV RepCap encoding polynucleotides; and (3) a vector that carries helper polynucleotides. One of skill in the art will appreciate various methods and variations thereof that are both helper- and helper-free and as well as the different advantages of each system.

### Non-Viral Vectors

In an embodiment, the vector is a non-viral vector or vector system. The term of art “Non-viral vector” and as used herein in this context refers to molecules and/or compositions that are vectors but that are not based on one or more components of a virus or virus genome (excluding any nucleotide to be delivered and/or expressed by the non-viral vector) that can be capable of incorporating engineered Acr delivery system polynucleotide(s) and/or CRISPR-Cas polynucleotide(s) and delivering said engineered Acr delivery system polynucleotide(s) and/or CRISPR-Cas polynucleotide(s) to a cell and/or expressing the polynucleotide in the cell. It will be appreciated that this does not exclude vectors containing a polynucleotide designed to target a virus-based polynucleotide that is to be delivered. For example, if a gRNA to be delivered is directed against a virus component and it is inserted or otherwise coupled to an otherwise non-viral vector or carrier, this would not make said vector a “viral vector”. Non-viral vectors can include, without limitation, naked polynucleotides and polynucleotide (non-viral) based vector and vector systems.

### Naked Polynucleotides

In an embodiment one or more engineered Acr delivery system polynucleotide(s) and/or CRISPR-Cas system polynucleotides described elsewhere herein can be included in a naked polynucleotide. The term of art “naked polynucleotide” as used herein refers to polynucleotides that are not associated with another molecule (e.g., proteins, lipids, and/or other molecules) that can often help protect it from environmental factors and/or degradation. As used herein, associated with includes, but is not limited to, linked to, adhered to, adsorbed to, enclosed in, enclosed in or within, mixed with, and the like. Naked polynucleotides that include one or more of the engineered Acr delivery system polynucleotide(s) and/or CRISPR-Cas system polynucleotides described herein can be delivered directly to a host cell and optionally expressed therein. The naked polynucleotides can have any suitable two- and three-dimensional configurations. By way of non-limiting examples, naked polynucleotides can be single-stranded molecules, double-stranded molecules, circular molecules (e.g., plasmids and artificial chromosomes), molecules that contain portions that are single-stranded and portions that are double-stranded (e.g. ribozymes), and the like. In an embodiment, the naked polynucleotide contains only the engineered Acr delivery system polynucleotide(s) and/or CRISPR-Cas system polynucleotide(s) of the present invention. In an embodiment, the naked polynucleotide can contain other nucleic acids and/or polynucleotides in addition to the engineered Acr delivery system polynucleotide(s) and/or CRISPR-Cas system polynucleotide(s) of the present invention. The naked polynucleotides can include one or more elements of a transposon system. Transposons and systems thereof are described in greater detail elsewhere herein.

### Non-Viral Polynucleotide Vectors

In an embodiment, one or more of the engineered Acr delivery system polynucleotide(s) and/or CRISPR-Cas system polynucleotides can be included in a non-viral polynucleotide vector. Suitable non-viral polynucleotide vectors include, but are not limited to, transposon vectors and vector systems, plasmids, bacterial artificial chromosomes, yeast artificial chromosomes, AR (antibiotic resistance)-free plasmids and miniplasmids, circular covalently closed vectors (e.g. minicircles, minivectors, miniknots), linear covalently closed vectors (“dumbbell-shaped”), MIDGE (minimalistic immunologically defined gene expression) vectors, MiLV (micro-linear vector) vectors, Ministrings, mini-intronic plasmids, PSK systems (post-segregationally killing systems), ORT (operator repressor titration) plasmids, and the like. See e.g., Hardee et al. 2017. Genes. 8(2):65.

In an embodiment, the non-viral polynucleotide vector can have a conditional origin of replication. In an embodiment, the non-viral polynucleotide vector can be an ORT plasmid. In an embodiment, the non-viral polynucleotide vector can have a minimalistic immunologically defined gene expression. In an embodiment, the non-viral polynucleotide vector can have one or more post-segregationally killing system genes. In an embodiment, the non-viral polynucleotide vector is AR-free. In an embodiment, the non-viral polynucleotide vector is a minivector. In an embodiment, the non-viral polynucleotide vector includes a nuclear localization signal. In an embodiment, the non-viral polynucleotide vector can include one or more CpG motifs. In an embodiment, the non-viral polynucleotide vectors can include one or more scaffold/matrix attachment regions (S/MARs). See e.g. Mirkovitch et al. 1984. Cell. 39:223-232, Wong et al. 2015. Adv. Genet. 89:113-152, whose techniques and vectors can be adapted for use in the present invention. S/MARs are AT-rich sequences that play a role in the spatial organization of chromosomes through DNA loop base attachment to the nuclear matrix. S/MARs are often found close to regulatory elements such as promoters, enhancers, and origins of DNA replication. The inclusion of one or more S/MARs can facilitate a once-per-cell-cycle replication to maintain the non-viral polynucleotide vector as an episome in daughter cells. In certain embodiments, the S/MAR sequence is located downstream of an actively transcribed polynucleotide (e.g. one or more Acr delivery system polynucleotide(s) and/or CRISPR-Cas system polynucleotide(s) co-therapy of the present invention) included in the non-viral polynucleotide vector. In an embodiment, the S/MAR can be a S/MAR from the beta-interferon gene cluster. See e.g. Verghese et al. 2014. Nucleic Acid Res. 42:e53; Xu et al. 2016. Sci. China Life Sci. 59:1024-1033; Jin et al. 2016. 8:702-711; Koirala et al. 2014. Adv. Exp. Med. Biol. 801:703-709; and Nehlsen et al. 2006. Gene Ther. Mol. Biol. 10:233-244, whose techniques and vectors can be adapted for use in the present invention.

In an embodiment, the non-viral vector is a transposon vector or system thereof. As used herein, “transposon” (also referred to as transposable element) refers to a polynucleotide sequence that is capable of moving from one location in a genome to another. There are several classes of transposons. Transposons include retrotransposons and DNA transposons. Retrotransposons require the transcription of the polynucleotide that is moved (or transposed) in order to transpose the polynucleotide to a new genome or polynucleotide. DNA transposons are those that do not require reverse transcription of the polynucleotide that is moved (or transposed) in order to transpose the polynucleotide to a new genome or polynucleotide. In an embodiment, the non-viral polynucleotide vector can be a retrotransposon vector. In an embodiment, the retrotransposon vector includes long terminal repeats. In an embodiment, the retrotransposon vector does not include long terminal repeats. In an embodiment, the non-viral polynucleotide vector can be a DNA transposon vector. DNA transposon vectors can include a polynucleotide sequence encoding a transposase. In an embodiment, the transposon vector is configured as a non-autonomous transposon vector, meaning that the transposition does not occur spontaneously on its own. In some of these embodiments, the transposon vector lacks one or more polynucleotide sequences encoding proteins required for transposition. In an embodiment, the non-autonomous transposon vectors lack one or more Ac transposable elements.

In an embodiment, a non-viral polynucleotide transposon vector system can include a first polynucleotide vector that contains the Acr delivery system polynucleotide(s) and/or CRISPR-Cas system co-therapy polynucleotide(s) of the present invention flanked on 5′ and 3′ ends by transposon terminal inverted repeats (TIRs) and a second polynucleotide vector that includes a polynucleotide capable of encoding a transposase coupled to a promoter to drive expression of the transposase. When both are expressed in the same cell the transposase can be expressed from the second vector and can transpose the material between the TIRs on the first vector (e.g. the Acr delivery system polynucleotide(s) and/or CRISPR-Cas system polynucleotide(s) of the present invention) and integrate it into one or more positions in the host cell's genome. In an embodiment, the transposon vector or system thereof can be configured as a gene trap. In an embodiment, the TIRs can be configured to flank a strong splice acceptor site followed by a reporter and/or another gene (e.g. one or more of the Acr delivery system polynucleotide(s) and/or CRISPR-Cas system polynucleotide(s) of the present invention) and a strong poly A tail. When transposition occurs while using this vector or system thereof, the transposon can insert into an intron of a gene and the inserted reporter or another gene can provoke a mis-splicing process and as a result, it inactivates the trapped gene.

Any suitable transposon system can be used. Suitable transposon and systems thereof can include Sleeping Beauty transposon system (Tc1/mariner superfamily) (see e.g. Ivics et al. 1997. Cell. 91 (4): 501-510), piggyBac (piggyBac superfamily) (see e.g. Li et al. 2013 110 (25): E2279-E2287 and Yusa et al. 2011. PNAS. 108 (4): 1531-1536), Tol2 (superfamily hAT), Frog Prince (Tc1/mariner superfamily) (see e.g. Miskey et al. 2003 Nucleic Acid Res. 31 (23): 6873-6881) and variants thereof.

### Delivery Vehicles

Described in certain example embodiments herein are delivery vehicles comprising (a) one or more CREs of the present invention, one or more engineered polynucleotides and/or gene products produced therefrom of the present invention, and/or one or more vectors or vector systems of the present invention described herein.

The delivery vehicles may deliver the one or more CREs of the present invention, one or more engineered polynucleotides and/or gene products produced therefrom of the present invention, and/or one or more vectors or vector systems of the present invention into and/or within effective proximity of cells, tissues, organs, or organisms (e.g., animals or plants). As used herein, the term “effective proximity” refers to the distance, region, or area surrounding a reference point, molecule, compound, or object in which a desired effect or activity occurs. The effective proximity can be determined by measuring the desired effect or activity in a representative number of species in the area surrounding the reference point or object. By way of non-limiting examples, an agent can be delivered to a specific point in a tissue of a subject and can be diffused through the surrounding tissue and cause effects in cells at a distance from the initial point of delivery. Cells that are affected by the agent can be determined and thus the region of effective proximity can be determined. Cells within that region are said to be within effective proximity to the initial delivery point. Similarly, if a cell is engineered to produce a product and secretes it into the surrounding environment, cells in the surrounding environment that are affected by the secreted product are said to be within effective proximity to the producing cell (or reference point). Likewise, if two (or more) molecules, compounds, compositions, objects, and/or the like are in effective proximity to one another, such a distance, region, or area can be defined and/or determined by measuring a change in one or more of the molecules, compounds, compositions, objects, and/or the like, a product produced from the molecules, compounds, compositions, objects, and/or the like (e.g., light, heat, or product compound, composition and/or the like). The molecules, compounds, compositions, objects, and/or the like are in “effective proximity” at the physical distance(s), position(s), etc. where a change, reaction, product, and/or the like is produced. In an embodiment, effective proximity ranges from 0 to 1, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1010, 1020, 1030, 1040, 1050, 1060, 1070, 1080, 1090, 1100, 1110, 1120, 1130, 1140, 1150, 1160, 1170, 1180, 1190, 1200, 1210, 1220, 1230, 1240, 1250, 1260, 1270, 1280, 1290, 1300, 1310, 1320, 1330, 1340, 1350, 1360, 1370, 1380, 1390, 1400, 1410, 1420, 1430, 1440, 1450, 1460, 1470, 1480, 1490, 1500, 1510, 1520, 1530, 1540, 1550, 1560, 1570, 1580, 1590, 1600, 1610, 1620, 1630, 1640, 1650, 1660, 1670, 1680, 1690, 1700, 1710, 1720, 1730, 1740, 1750, 1760, 1770, 1780, 1790, 1800, 1810, 1820, 1830, 1840, 1850, 1860, 1870, 1880, 1890, 1900, 1910, 1920, 1930, 1940, 1950, 1960, 1970, 1980, 1990, 2000 angstroms, pm, microns, or mm away from the reference point. In an embodiment, direct contact or bonding (i.e., effective proximity is 0).

In connection with delivery vehicles herein, the one or more CREs of the present invention, one or more engineered polynucleotides and/or gene products produced therefrom of the present invention, and/or one or more vectors or vector systems of the present invention that are carried by the delivery vehicle are referred to as “cargos” for simplicity, The cargos may be packaged, carried, or otherwise associated with the delivery vehicles. The delivery vehicles may be selected based on the types of cargo to be delivered, and/or the mode of delivery (e.g., in vitro and/or in vivo). Examples of delivery vehicles include vectors, viruses (e.g., virus particles), non-viral vehicles, and other delivery reagents described herein.

The delivery vehicles described herein can have a greatest dimension or greatest average dimension (e.g., diameter or greatest average diameter) of less than 100 microns (μm). In an embodiment, the delivery vehicles have a greatest dimension or greatest average dimension of less than 10 μm. In an embodiment, the delivery vehicles may have a greatest dimension or greatest average dimension of less than 2000 nanometers (nm). In an embodiment, the delivery vehicles may have a greatest dimension or greatest average dimension of less than 1000 nanometers (nm). In an embodiment, the delivery vehicles may have a greatest dimension or greatest average dimension (e.g., diameter or average diameter) of less than 900 nm, less than 800 nm, less than 700 nm, less than 600 nm, less than 500 nm, less than 400 nm, less than 300 nm, less than 200 nm, less than 150 nm, or less than 100 nm, less than 50 nm. In an embodiment, the delivery vehicles may have a greatest dimension or greatest average dimension ranging between 25 nm and 200 nm.

In an embodiment, the delivery vehicles may be or comprise particles. For example, the delivery vehicle may be or comprise nanoparticles (e.g., particles with a greatest dimension or greatest average dimension (e.g., diameter or greatest average diameter) no greater than 1000 nm. The particles may be provided in different forms, e.g., as solid particles (e.g., a metal such as silver, gold, iron, titanium), non-metal, lipid-based solids, polymers, suspensions of particles, or combinations thereof. Metal, dielectric, and semiconductor particles may be prepared, as well as hybrid structures (e.g., core-shell particles).

Nanoparticles may also be used to deliver the compositions and systems to cells, as described in WO 2008042156, US20130185823, and WO2015089419. In general, a “nanoparticle” refers to any particle having a diameter of less than 1000 nm. In certain embodiments, nanoparticles of the invention have a greatest dimension or greatest average dimension (e.g., diameter or average diameter) of 500 nm or less. In other embodiments, nanoparticles of the invention have a greatest dimension or greatest average dimension ranging between 25 nm and 200 nm. In other embodiments, nanoparticles of the invention have a greatest dimension or greatest average dimension of 100 nm or less. In other embodiments, nanoparticles of the invention have a greatest dimension or greatest average dimensions ranging between 35 nm and 60 nm. It will be appreciated that reference made herein to particles or nanoparticles can be interchangeable, where appropriate. Nanoparticles made of semiconducting material may also be labeled quantum dots if they are small enough (typically sub 10 nm) that quantization of electronic energy levels occurs. Such nanoscale particles are used in biomedical applications as drug carriers or imaging agents and may be adapted for similar purposes in the present invention. Semi-solid and soft nanoparticles have been manufactured and are within the scope of the present invention. Nanoparticles with one-half hydrophilic and the other half hydrophobic are termed Janus particles and are particularly effective for stabilizing emulsions. They can self-assemble at water/oil interfaces and act as solid surfactants.

Particle characterization (including e.g., characterizing morphology, dimension, etc.) is done using a variety of different techniques. Common techniques are electron microscopy (TEM, SEM), atomic force microscopy (AFM), dynamic light scattering (DLS), X-ray photoelectron spectroscopy (XPS), powder X-ray diffraction (XRD), Fourier transform infrared spectroscopy (FTIR), matrix-assisted laser desorption/ionization time-of-flight mass spectrometry (MALDI-TOF MS), ultraviolet-visible spectroscopy, dual polarization interferometry and nuclear magnetic resonance (NMR). Characterization (dimension measurements) may be made as to native particles (i.e., preloading) or after loading of the cargo (herein cargo refers to e.g., one or more CREs of the present invention, one or more engineered polynucleotides and/or gene products produced therefrom of the present invention, and/or one or more vectors or vector systems of the present invention or any other system described herein e.g., CRISPR-Cas system e.g., CRISPR enzyme or mRNA or guide RNA, or any combination thereof, and may include additional carriers and/or excipients) to provide particles of an optimal size for delivery for any in vitro, ex vivo and/or in vivo application of the present invention. In certain preferred embodiments, particle dimension (e.g., diameter) characterization is based on measurements using dynamic laser scattering (DLS). Mention is made of U.S. Pat. Nos. 8,709,843; 6,007,845; 5,855,913; 5,985,309; 5,543,158; and the publication by James E. Dahlman and Carmen Barnes et al. Nature Nanotechnology (2014) published online 11 May 2014, doi: 10.1038/nnano.2014.84, describing particles, methods of making and using them, and measurements thereof.

### Vectors and Vector Systems

In an embodiment, the delivery vehicle is a vector or vector system. Vectors and vector systems of the present invention are described in greater detail elsewhere herein.

### Non-Vector Delivery Vehicles

The delivery vehicles may comprise non-viral vehicles. In general, methods and vehicles capable of delivering nucleic acids and/or proteins may be used for delivering the systems compositions herein. Examples of non-viral vehicles include lipid nanoparticles, cell-penetrating peptides (CPPs), DNA nanoclews, metal nanoparticles, streptolysin O, multifunctional envelope-type nanodevices (MENDs), lipid-coated mesoporous silica particles, and other inorganic nanoparticles, and those systems described in Hirschenberger et al. 2021. Front. Pharmacol. 12:770283. doi: 10.3389/fphar.2021.770283 and Tian et al., Cell. Rep. 38 (10): 110476 (2022)

### Lipid Particles

The delivery vehicles may comprise lipid particles, e.g., lipid nanoparticles (LNPs) and liposomes. Lipofection is described in e.g., U.S. Pat. Nos. 5,049,386, 4,946,787; and 4,897,355) and lipofection reagents are sold commercially (e.g., Transfectam™ and Lipofectin™). Cationic and neutral lipids that are suitable for efficient receptor-recognition lipofection of polynucleotides include those of Felgner, International Patent Publication Nos. WO 91/17424 and WO 91/16024. The preparation of lipid: nucleic acid complexes, including targeted liposomes such as immunolipid complexes, is well known to one of skill in the art (see, e.g., Crystal, Science 270:404-410 (1995); Blaese et al., Cancer Gene Ther. 2:291-297 (1995); Behr et al., Bioconjugate Chem. 5:382-389 (1994); Remy et al., Bioconjugate Chem. 5:647-654 (1994); Gao et al., Gene Therapy 2:710-722 (1995); Ahmad et al., Cancer Res. 52:4817-4820 (1992); U.S. Pat. Nos. 4,186,183, 4,217,344, 4,235,871, 4,261,975, 4,485,054, 4,501,728, 4,774,085, 4,837,028, and 4,946,787).

### Lipid Nanoparticles (LNPs)

LNPs may encapsulate nucleic acids within cationic lipid particles (e.g., liposomes), and may be delivered to cells with relative ease. In some examples, lipid nanoparticles do not contain any viral components, which helps minimize safety and immunogenicity concerns. Lipid particles may be used for in vitro, ex vivo, and in vivo deliveries. Lipid particles may be used for various scales of cell populations.

In some examples. LNPs may be used for delivering DNA molecules (e.g., those comprising coding sequences of Cas and/or gRNA) and/or RNA molecules (e.g., mRNA of Cas, gRNAs). In an embodiment, LNPs can include and be used to deliver the cargos described herein, which include, but are not limited to one or more CREs of the present invention, one or more engineered polynucleotides and/or gene products produced therefrom of the present invention, and/or one or more vectors or vector systems of the present invention, a CRISPR-Cas system or component thereof and other gene products. In certain cases, LNPs may be used for delivering RNP complexes that can be composed of one or more gene products, including but not limited to CRISPR-Cas system components.

Components in LNPs may comprise cationic lipids 1,2-dilineoyl-3-dimethylammonium-propane (DLinDAP), 1,2-dilinoleyloxy-3-N,N-dimethylaminopropane (DLinDMA), 1,2-dilinoleyloxyketo-N,N-dimethyl-3-aminopropane (DLinK-DMA), 1,2-dilinoleyl-4-(2-dimethylaminoethyl)-[1,3]-dioxolane (DLinKC2-DMA), (3-o-[2″-(methoxypolyethyleneglycol 2000) succinoyl]-1,2-dimyristoyl-sn-glycol (PEG-S-DMG), R-3-[(ro-methoxy-poly(ethylene glycol) 2000) carbamoyl]-1,2-dimyristyloxlpropyl-3-amine (PEG-C-DOMG), and any combination thereof. Preparation of LNPs and encapsulation may be adapted from Rosin et al, Molecular Therapy, vol. 19, no. 12, pages 1286-220 Dec. 2011.

In an embodiment, an LNP delivery vehicle can be used to deliver a virus particles, virus-like particles, proteins, and/or polynucleotides (e.g., DNA, RNA (e.g., mRNA), or ribonucleoprotein (RNP) complex, or one or more other cargos, including but not limited to, one or more CREs of the present invention, one or more engineered polynucleotides and/or gene products produced therefrom of the present invention, and/or one or more vectors or vector systems of the present invention. In an embodiment, the virus particle(s), polynucleotide, and/or RNP can be adsorbed to the lipid particle, such as through electrostatic interactions, and/or can be attached to the liposomes via a linker.

In an embodiment, the LNP contains a nucleic acid, wherein the charge ratio of nucleic acid backbone phosphates to cationic lipid nitrogen atoms is about 1:1.5-7 or about 1:4.

In an embodiment, the LNP also includes a shielding compound, which is removable from the lipid composition under in vivo conditions. In an embodiment, the shielding compound is a biologically-inert compound. In an embodiment, the shielding compound does not carry any charge on its surface or on the molecule as such. In an embodiment, the shielding compounds are polyethylenglycoles (PEGs), hydroxyethylglucose (HEG) based polymers, polyhydroxyethyl starch (polyHES), and/or polypropylene. In an embodiment, the PEG, HEG, polyHES, and polypropylene weigh between about 500 to 10,000 Da or between about 2000 to 5000 Da. In an embodiment, the shielding compound is PEG2000 or PEG5000.

In an embodiment, the LNP can include one or more helper lipids. In an embodiment, the helper lipid can be a phospholipid or a steroid. In an embodiment, the helper lipid is between about 20 mol % to 80 mol % of the total lipid content of the composition. In an embodiment, the helper lipid component is between about 35 mol % to 65 mol % of the total lipid content of the LNP. In an embodiment, the LNP includes lipids at 50 mol % of the LNP, of which the helper lipid is present at 50 mol % of the total lipid content of the LNP.

Other non-limiting, exemplary LNP delivery vehicles are described in U.S. Patent Publication Nos. US20160174546, US20140301951, US20150105538, US20150250725, Wang et al., J. Control Release, 2017 Jan. 31. pii: S0168-3659 (17) 30038-X. doi: 10.1016/j.jconrel.2017.01.037. [Epub ahead of print]; Altnoǧlu et al., Biomater Sci., 4 (12): 1773-80, Nov. 15, 2016; Wang et al., PNAS, 113 (11): 2868-73 Mar. 15, 2016; Wang et al., PloS One, 10 (11): e0141860. doi: 10.1371/journal.pone.0141860. eCollection 2015 Nov. 3, 2015; Takeda et al., Neural Regen Res. 10 (5): 689-90, May 2015; Wang et al., Adv. Healthc Mater., 3 (9): 1398-403, September 2014; and Wang et al., Agnew Chem Int Ed Engl., 53 (11): 2893-8, Mar. 10, 2014; James E. Dahlman and Carmen Barnes et al. Nature Nanotechnology (2014) published online 11 May 2014, doi: 10.1038/nnano.2014.84; Coelho et al., N Engl J Med 2013; 369:819-29; Aleku et al., Cancer Res., 68 (23): 9788-98 (Dec. 1, 2008), Strumberg et al., Int. J. Clin. Pharmacol. Ther., 50 (1): 76-8 (January 2012), Schultheis et al., J. Clin. Oncol., 32 (36): 4141-48 (Dec. 20, 2014), and Fehring et al., Mol. Ther., 22 (4): 811-20 (Apr. 22, 2014); Novobrantseva, Molecular Therapy-Nucleic Acids (2012) 1, e4; doi: 10.1038/mtna.2011.3; WO2012135025; US20140348900; US20140328759; US 20140308304; WO 2005/105152; WO 2006/069782; WO 2007/121947; US 2015/082080; US 20120251618; 7,982,027; 7,799,565; 8,058,069; 8,283,333; 7,901,708; 7,745,651; 7,803,397; 8,101,741; 8,188,263; 7,915,399; 8,236,943 and 7,838,658 and European Pat. Nos 1766035; 1519714; 1781593 and 1664316.

### Liposomes

In an embodiment, a lipid particle may be a liposome. Liposomes are spherical vesicle structures composed of a uni- or multi-lamellar lipid bilayer surrounding internal aqueous compartments and a relatively impermeable outer lipophilic phospholipid bilayer. In an embodiment, liposomes are biocompatible, nontoxic, can deliver both hydrophilic and lipophilic drug molecules, protect their cargo from degradation by plasma enzymes, and transport their load across biological membranes and the blood-brain barrier (BBB).

Liposomes can be made from several different types of lipids, e.g., phospholipids. A liposome may comprise natural phospholipids and lipids such as 1,2-distearoryl-sn-glycero-3-phosphatidyl choline (DSPC), sphingomyelin, egg phosphatidylcholines, monosialoganglioside, or any combination thereof.

Several other additives may be added to liposomes in order to modify their structure and properties. For instance, liposomes may further comprise cholesterol, sphingomyelin, and/or 1,2-dioleoyl-sn-glycero-3-phosphoethanolamine (DOPE), e.g., to increase stability and/or to prevent the leakage of the liposomal inner cargo.

In an embodiment, a liposome delivery vehicle can be used to deliver a virus particle, vector, polynucleotide and/or protein, and/or complex thereof (e.g., an RNP) containing a CRISPR-Cas system and/or component(s) thereof or one or more other gene products. In an embodiment, the virus particle(s) can be adsorbed to the liposome, such as through electrostatic interactions, and/or can be attached to the liposomes via a linker.

In an embodiment, the liposome can be a Trojan Horse liposome (also known in the art as Molecular Trojan Horses), see e.g., http://cshprotocols.cshlp.org/content/2010/4/pdb.prot5407.long, the teachings of which can be applied and/or adapted to generate and/or deliver the cargos described herein.

Other non-limiting, exemplary liposomes can be those as set forth in Wang et al., ACS Synthetic Biology, 1, 403-07 (2012); Wang et al., PNAS, 113 (11) 2868-2873 (2016); Spuch and Navarro, Journal of Drug Delivery, vol. 2011, Article ID 469679, 12 pages, 2011. doi: 10.1155/2011/469679; WO 2008/042973; U.S. Pat. No. 8,071,082; WO 2014/186366; 20160257951; US20160129120; US20160244761; 20120251618; WO2013/093648; Lipofectin (a combination of DOTMA and DOPE), Lipofectase, LIPOFECTAMINE® (e.g., LIPOFECTAMINE®. 2000, LIPOFECTAMINE® 3000, LIPOFECTAMINE® RNAIMAX, LIPOFECTAMINE® LTX), SAINT-RED (Synvolux Therapeutics, Groningen Netherlands), DOPE, Cytofectin (Gilead Sciences, Foster City, Calif.), and Eufectins (JBL, San Luis Obispo, Calif.).

### Stable Micleic-Acid-Lipid Particles (SNALPs)

In an embodiment, the lipid particles may be stable nucleic-acid-lipid particles (SNALPs). SNALPs may comprise an ionizable lipid (e.g., DLinDMA, which iscationic at low pH), a neutral helper lipid (e.g., cholesterol), a diffusible polyethylene glycol (PEG)-lipid, or any combination thereof. In some examples, SNALPs may comprise synthetic cholesterol, dipalmitoylphosphatidylcholine, 3-N-[(w-methoxy polyethylene glycol) 2000) carbamoyl]-1,2-dimyrestyloxypropylamine, and cationic 1,2-dilinoleyloxy-3-N,Ndimethylaminopropane. In some examples, SNALPs may comprise synthetic cholesterol, 1,2-distearoyl-sn-glycero-3-phosphocholine, PEG-CDMA, and 1,2-dilinoleyloxy-3-(N,N-dimethyl)aminopropane (DLinDMAo).

Other non-limiting, exemplary SNALPs that can be used to deliver cargos described herein can be any such SNALPs as described in Morrissey et al., Nature Biotechnology, Vol. 23, No. 8, August 2005, Zimmerman et al., Nature Letters, Vol. 441, 4 May 2006; Geisbert et al., Lancet 2010; 375:1896-905; Judge, J. Clin. Invest. 119:661-673 (2009); and Semple et al., Nature Biotechnology, Volume 28 Number 2 Feb. 2010, pp. 172-177. In an embodiment, the cargos are an RNP, such as a CRISPR-Cas RNP. In other embodiments, the cargo is included as mRNA.

### Other Lipids

The lipid particles may also comprise one or more other types of lipids, e.g., cationic lipids, such as amino lipid 2,2-dilinoleyl-4-dimethylaminoethyl-[1,3]-dioxolane (DLin-KC2-DMA), DLin-KC2-DMA4, C12-200, and co-lipids disteroylphosphatidyl choline, cholesterol, and PEG-DMG.

In an embodiment, the delivery vehicle can be or include a lipidoid, such as any of those set forth in, for example, US20110293703.

In an embodiment, the delivery vehicle can be or include an amino lipid, such as any of those set forth in, for example, Jayaraman, Angew. Chem. Int. Ed. 2012, 51, 8529-8533.

In an embodiment, the delivery vehicle can be or include a lipid envelope, such as any of those set forth in, for example, Korman et al., 2011. Nat. Biotech. 29:154-157.

### Lipoplexes Polyplexes

In an embodiment, the delivery vehicles comprise lipoplexes and/or polyplexes. Lipoplexes may bind to negatively charged cell membranes and induce endocytosis into the cells. Examples of lipoplexes may be complexes comprising lipid(s) and non-lipid components. Examples of lipoplexes and polyplexes include FuGENE-6 reagent, a non-liposomal solution containing lipids and other components, zwitterionic amino lipids (ZALs), Ca2 [symbol] (e.g., forming DNA/Ca²⁺ microcomplexes), polyethenimine (PE1) (e.g., branched PE1), and poly(L-lysine) (PLL).

### Sugar-Based Particles

In an embodiment, the delivery vehicle can be a sugar-based particle. In an embodiment, the sugar-based particles can be or include GalNAc, such as any of those described in WO2014118272; US20020150626; Nair, J K et al., 2014, Journal of the American Chemical Society 136 (49), 16958-16961; Østergaard et al., Bioconjugate Chem., 2015, 26 (8), pp 1451-1455.

### Cell-Penetrating Peptides

In an embodiment, the delivery vehicles comprise cell-penetrating peptides (CPPs). CPPs are short peptides that facilitate cellular uptake of various molecular cargos (e.g., from nanosized particles to small chemical molecules and large fragments of DNA).

CPPs may be of different sizes, amino acid sequences, and charges. In some examples, CPPs can translocate the plasma membrane and facilitate the delivery of various molecular cargos to the cytosolor an organelle. CPPs may be introduced into cells via different mechanisms, e.g., direct penetration in the membrane, endocytosis-mediated entry, and translocation through the formation of a transitory structure.

CPPs may have an amino acid composition that either contains a high relative abundance of positively charged amino acids such as lysine or arginine or has sequences that contain an alternating pattern of polar/charged amino acids and non-polar, hydrophobic amino acids. These two types of structures are referred to as polycationic or amphipathic, respectively. A third class of CPPs is the hydrophobic peptides, containing only apolar residues, with low net charge or with hydrophobic amino acid groups that are crucial for cellular uptake. Another type of CPPs is the trans-activating transcriptional activator (Tat) from Human Immunodeficiency Virus 1 (HIV-1). Examples of CPPs include Penetratin, Tat (48-60), Transportan, and (R-AhX-R4) (Ahx refers to aminohexanoyl), Kaposi fibroblast growth factor (FGF) signal peptide sequence, integrin β3 signal peptide sequence, polyarginine peptide (poly-Arg) sequence, Guanine rich-molecular transporters, and sweet arrow peptide. In an embodiment, the CPP is a cyclic CPP (see e.g., Herce et al., Nat. Chem.9:762-771 (2017)). Examples of CPPs and related applications also include those described in U.S. Pat. No. 8,372,951.

CPPs can be used for in vitro and ex vivo work quite readily, and extensive optimization for each cargo and cell type is usually required. In some examples, CPPs may be covalently attached to the Cas protein directly, which is then complexed with the gRNA and delivered to cells. See e.g., Ramakrishna et al. Genome Res. 2014. 24:1020-1027 and Staahl et al. Nature Biotechnology. 35:431-434 (2017). In some examples, separate delivery of CPP-Cas and CPP-gRNA to multiple cells may be performed. CPPs may also be used to deliver RNPs.

CPPs may be used to deliver the compositions and systems to plants. In some examples, CPPs may be used to deliver the components to plant protoplasts, which are then regenerated to plant cells and further to plants.

### DNA Nanoclews

In an embodiment, the delivery vehicles comprise DNA nanoclews. A DNA nanoclew refers to a sphere-like structure of DNA (e.g., with a shape of a ball of yarn). The nanoclew may be synthesized by rolling circle amplification with palindromic sequences that aid in the self-assembly of the structure. The sphere may then be loaded with a payload. An example of DNA nanoclew is described in Sun W et al, J Am Chem Soc. 2014 Oct. 22; 136 (42): 14722-5; and Sun W et al, Angew Chem Int Ed Engl. 2015 Oct. 5; 54 (41): 12029-33. A DNA nanoclew may have a palindromic sequence to be partially complementary to the gRNA within the Cas:gRNA ribonucleoprotein complex. A DNA nanoclew may be coated, e.g., coated with PE1 to induce endosomal escape.

### Metal Nanoparticles

In an embodiment, the delivery vehicles comprise gold nanoparticles (also referred to AuNPs or colloidal gold). Gold nanoparticles may form a complex with cargos, e.g., Cas:gRNA RNP. Gold nanoparticles may be coated, e.g., coated in a silicate and an endosomal disruptive polymer, PAsp (DET). Examples of gold nanoparticles include AuraSense Therapeutics' Spherical Nucleic Acid (SNA™) constructs, and those described in Mout R, et al. (2017). ACS Nano 11:2452-8; Lee K, et al. (2017). Nat Biomed Eng 1:889-901. Other metal nanoparticles can also be complexed with cargo(s). Such metal particles include tungsten, palladium, rhodium, platinum, and iridium particles. Other non-limiting, exemplary metal nanoparticles are described in US20100129793.

iTOP

In an embodiment, the delivery vehicles comprise iTOP. iTOP refers to a combination of small molecules that drives the highly efficient intracellular delivery of native proteins, independent of any transduction peptide. iTOP may be used for induced transduction by osmocytosis and propanebetaine, using NaCl-mediated hyperosmolality together with a transduction compound (propanebetaine) to trigger macropinocytotic uptake into cells of extracellular macromolecules. Examples of iTOP methods and reagents include those described in D'Astolfo D S, Pagliero R J, Pras A, et al. (2015). Cell 161:674-690.

### Polymer-Based Particles

In an embodiment, the delivery vehicles may comprise polymer-based particles (e.g., nanoparticles). In an embodiment, the polymer-based particles may mimic a viral mechanism of membrane fusion. The polymer-based particles may be a synthetic copy of Influenza virus machinery and form transfection complexes with various types of nucleic acids (siRNA, miRNA, plasmid DNA, shRNA, or mRNA) that cells take up via the endocytosis pathway, a process that involves the formation of an acidic compartment. The low pH in late endosomes acts as a chemical switch that renders the particle surface hydrophobic and facilitates membrane crossing. Once in the cytosol, the particle releases its payload for cellular action. This Active Endosome Escape technology is safe and maximizes transfection efficiency as it is using a natural uptake pathway. In an embodiment, the polymer-based particles may comprise alkylated and carboxyalkylated branched polyethylenimine. In some examples, the polymer-based particles are or comprise Viromers, e.g., ViromerR RNAi, Viromer RED, Viromer mRNA, Viromer CRISPR. Example methods of delivering the systems and compositions herein include those described in Bawage S S et al., Synthetic mRNA expressed Cas13a mitigates RNA virus infections, biorxiv.org/content/10.1101/370460v1.full doi: doi.org/10.1101/370460, Viromer® RED, a powerful tool for transfection of keratinocytes. doi: 10.13140/RG.2.2.16993.61281, Viromer® Transfection-Factbook 2018: technology, product overview, users' data., doi: 10.13140/RG.2.2.23912.16642. Other exemplary and non-limiting polymeric particles are described in US20170079916, US20160367686, US 20110212179, US20130302401, U.S. Pat. Nos. 6,007,845, 5,855,913, 5,985,309, 5,543,158, WO2012135025, US20130252281, US20130245107, US20130244279; US20050019923, 20080267903.

### Streptolysin O (SLO)

The delivery vehicles may be streptolysin O (SLO). SLO is a toxin produced by Group A streptococci that works by creating pores in mammalian cell membranes. SLO may act in a reversible manner, which allows for the delivery of proteins (e.g., up to 100 kDa) to the cytosol of cells without compromising overall viability. Examples of SLO include those described in Sierig G, et al. (2003). Infect Immun 71:446-55; Walev I, et al. (2001). Proc. Natl. Acad. Sci U.S.A. 98:3185-90; Teng K W, et al. (2017). Elife 6:e25460.

### Multifunctional Envelope-Type Nanodevice (MEND)

The delivery vehicles may comprise multifunctional envelope-type nanodevices (MENDs). MENDs may comprise condensed plasmid DNA, a PLL core, and a lipid film shell. A MEND may further comprise a cell-penetrating peptide (e.g., stearyl octaarginine). The cell-penetrating peptide may be in the lipid shell. The lipid envelope may be modified with one or more functional components, e.g., one or more of: polyethylene glycol (e.g., to increase vascular circulation time), ligands for targeting specific tissues/cells, additional cell-penetrating peptides (e.g., for greater cellular delivery), lipids to enhance endosomal escape, and nuclear delivery tags. In some examples, the MEND may be a tetra-lamellar MEND (T-MEND), which may target the cellular nucleus and mitochondria. In certain examples, a MEND may be a PEG-peptide-DOPE-conjugated MEND (PPD-MEND), which may target bladder cancer cells. Examples of MENDs include those described in Kogure K, et al. (2004). J Control Release 98:317-23; Nakamura T, et al. (2012). Acc Chem Res 45:1113-21.

### Lipid-Coated Mesoporous Silica Particles

The delivery vehicles may comprise lipid-coated mesoporous silica particles. Lipid-coated mesoporous silica particles may comprise a mesoporous silica nanoparticle core and a lipid membrane shell. The silica core may have a large internal surface area, leading to high cargo loading capacities. In an embodiment, pore sizes, pore chemistry, and overall particle sizes may be modified for loading different types of cargo. The lipid coating of the particle may also be modified to maximize cargo loading, increase circulation times, and provide precise targeting and cargo release. Examples of lipid-coated mesoporous silica particles include those described in Du X, et al. (2014). Biomaterials 35:5580-90; Durfee P N, et al. (2016). ACS Nano 10:8325-45.

### Inorganic Nanoparticles

The delivery vehicles may comprise inorganic nanoparticles. Examples of inorganic nanoparticles include carbon nanotubes (CNTs) (e.g., as described in Bates K and Kostarelos K. (2013). Adv Drug Deliv Rev 65:2023-33.), bare mesoporous silica nanoparticles (MSNPs) (e.g., as described in Luo G F, et al. (2014). Sci Rep 4:6064), and dense silica nanoparticles (SiNPs) (as described in Luo D and Saltzman W M. (2000). Nat Biotechnol 18:893-5).

### Exosomes

The delivery vehicles may comprise exosomes. Exosomes include membrane-bound extracellular vesicles, which can be used to contain and deliver various types of biomolecules, such as proteins, carbohydrates, lipids, nucleic acids, and complexes thereof (e.g., RNPs). Examples of exosomes include those described in Schroeder A, et al., J. Intern Med. 2010 January; 267 (1): 9-21; E1-Andaloussi S, et al., Nat Protoc. 2012 December; 7 (12): 2112-26; Uno Y, et al., Hum Gene Ther. 2011 June; 22 (6): 711-9; Zou W, et al., Hum Gene Ther. 2011 April; 22 (4): 465-75.

In some examples, the exosome may form a complex (e.g., by binding directly or indirectly) to one or more components of the cargo. In certain examples, a molecule of an exosome may be fused with a first adapter protein and a component of the cargo may be fused with a second adapter protein. The first and the second adapter protein may specifically bind each other, thus associating the cargo with the exosome. Examples of such exosomes include those described in Ye Y, et al., Biomater Sci. 2020 Apr. 28. doi: 10.1039/d0bm00427h.

Other non-limiting, exemplary exosomes include any of those set forth in Alvarez-Erviti et al. 2011, Nat Biotechnol 29:341; E1-Andaloussi et al. (Nature Protocols 7:2112-2126 (2012); and Wahlgren et al. (Nucleic Acids Research, 2012, Vol. 40, No. 17 e130).

### Spherical Nucleic Acids (SNAs)

In an embodiment, the delivery vehicle can be an SNA. SNAs are three-dimensional nanostructures that can be composed of densely functionalized and highly oriented nucleic acids that can be covalently attached to the surface of spherical nanoparticle cores. The core of the spherical nucleic acid can impart the conjugate with specific chemical and physical properties, and it can act as a scaffold for assembling and orienting the oligonucleotides into a dense spherical arrangement that gives rise to many of their functional properties, distinguishing them from all other forms of matter. In an embodiment, the core is a crosslinked polymer. Non-limiting, exemplary SNAs can be any of those set forth in Cutler et al., J. Am. Chem. Soc. 2011 133:9254-9257, Hao et al., Small. 2011 7:3158-3162, Zhang et al., ACS Nano. 2011 5:6962-6970, Cutler et al., J. Am. Chem. Soc. 2012 134:1376-1391, Young et al., Nano Lett. 2012 12:3867-71, Zheng et al., Proc. Natl. Acad. Sci. USA. 2012 109:11975-80, Mirkin, Nanomedicine 2012 7:635-638 Zhang et al., J. Am. Chem. Soc. 2012 134:16488-1691, Weintraub, Nature 2013 495:S14-S16, Choi et al., Proc. Natl. Acad. Sci. USA. 2013 110 (19): 7625-7630, Jensen et al., Sci. Transl. Med. 5, 209ra152 (2013) and Mirkin, et al., and Small, 10:186-192.

### Self-Assembling Nanoparticles

In an embodiment, the delivery vehicle is a self-assembling nanoparticle. The self-assembling nanoparticles can contain one or more polymers. The self-assembling nanoparticles can be PEGylated. Self-assembling nanoparticles are known in the art. Non-limiting, exemplary self-assembling nanoparticles can be any as set forth in Schiffelers et al., Nucleic Acids Research, 2004, Vol. 32, No. 19, Bartlett et al. Proc. Natl. Acad. Sci. USA. Sep. 25, 2007, vol. 104, no. 39; Davis et al., Nature, Vol 464, 15 Apr. 2010.

### Supercharged Proteins

In an embodiment, the delivery vehicle can be a supercharged protein. As used herein “Supercharged proteins” are a class of engineered or naturally occurring proteins with unusually high positive or negative net theoretical charge. Non-limiting, exemplary supercharged proteins can be any of those set forth in Lawrence et al., 2007, Journal of the American Chemical Society 129, 10110-10112 and Fuchs and Raines. ACS Chem. Biol. 2 (3): 167-170 (2007).

### Virus-Like Particles

In an embodiment, the delivery vehicle can be a virus like particles. VLPs is a term of art that refers to particles produced from virus proteins, such as capsid or other proteins, but that do not contain the native viral genetic materials. Exemplary VLPs and their production systems and vectors for delivery of a cargo of the present invention described herein are described in e.g., Bhat et al., Viruses 14 (2): 383 (2022) doi: 10.3390/v14020383; Hill et al., Curr Protein Pept Sci. (2018) 19 (1): 112-127; Schwarz B et al., Adv Virus Res. 2017. 97:1-60 doi: 10.1016/bs.aivir.2016.09.002; Banskota et al., Cell. 2022. 185 (2): 250-265; Ikwuagwu and Tullman-Ercek. Curr Opin Biotechnol. 2022. 78:102785 doi: 10.1016/j.copbio.2022.102785; Zdanowicz and Chroboczek. Acta Biochim Pol. 2016: 63 (3): 469-473; Suffian and Al-Jamal et al., Adv. Drug Deliv. Rev. 2022. 180:114030 doi: 10.1016/j.addr.2021.114030; and Segel et al., Science. 373:6557 (2021).

### Targeted Delivery

In an embodiment, the delivery vehicle can allow for targeted delivery to a specific cell, tissue, organ, or system. In such embodiments, the delivery vehicle can include one or more targeting moieties that can direct targeted delivery of the cargo(s). In an embodiment, the delivery vehicle comprises a targeting moiety.

Exemplary targeting moieties are described in greater detail elsewhere herein and are applicable to targeting moieties that can be included in a delivery vehicle.

### Responsive Delivery

In an embodiment, the delivery vehicle can allow for responsive delivery of the cargo(s). Responsive delivery, as used in this context herein, refers to delivery of cargo(s) by the delivery vehicle in response to an external stimulus. Examples of suitable stimuli include, without limitation, energy (light, heat, cold, and the like), chemical stimuli (e.g., chemical composition, etc.), and biologic or physiologic stimuli (e.g., environmental pH, osmolarity, salinity, biologic molecule, etc.). In an embodiment, the targeting moiety can be responsive to external stimuli and facilitate responsive delivery. In other embodiments, responsiveness is determined by a non-targeting moiety component of the delivery vehicle.

The delivery vehicle can be stimuli-sensitive, e.g., sensitive to externally applied stimuli, such as magnetic fields, ultrasound, or light; and pH-triggering can also be used, e.g., a labile linkage can be used between a hydrophilic moiety such as PEG and a hydrophobic moiety such as a lipid entity of the invention, which is cleaved only upon exposure to the relatively acidic conditions characteristic of a particular environment or microenvironment such as an endocytic vacuole or the acidotic tumor mass. pH-sensitive copolymers can also be incorporated in embodiments of the invention to provide shielding; diortho esters, vinyl esters, cysteine-cleavable lipopolymers, double esters, and hydrazones are a few examples of pH-sensitive bonds that are quite stable at pH 7.5, but are hydrolyzed relatively rapidly at pH 6 and below, e.g., a terminally alkylated copolymer of N-isopropylacrylamide and methacrylic acid that facilitates destabilization of a lipid entity of the invention and release in compartments with decreased pH value; or, the invention comprehends ionic polymers for generation of a pH-responsive lipid entity of the invention (e.g., poly(methacrylic acid), poly(diethylaminoethyl methacrylate), poly(acrylamide) and poly(acrylic acid)).

Temperature-triggered delivery is also within the ambit of the invention. Many pathological areas, such as inflamed tissues and tumors, show distinctive hyperthermia compared with normal tissues. Utilizing this hyperthermia is an attractive strategy in cancer therapy since hyperthermia is associated with increased tumor permeability and enhanced uptake. This technique involves local heating of the site to increase microvascular pore size and blood flow, which, in turn, can result in increased extravasation of embodiments of the invention. A temperature-sensitive lipid entity of the invention can be prepared from thermosensitive lipids or polymers with a low critical solution temperature. Above the low critical solution temperature (e.g., at a site such as the tumor site or inflamed tissue site), the polymer precipitates, disrupting the liposomes to release the cargo. Lipids with a specific gel-to-liquid phase transition temperature are used to prepare these lipid entities of the invention, and a lipid for a thermosensitive embodiment can be dipalmitoylphosphatidylcholine. Thermosensitive polymers can also facilitate destabilization followed by release, and a useful thermosensitive polymer is poly(N-isopropylacrylamide). Another temperature-triggered system can employ lysolipid temperature-sensitive liposomes.

The invention also comprehends redox-triggered delivery. The difference in redox potential between normal and inflamed or tumor tissues, and between the intra- and extracellular environments has been exploited for delivery, e.g., glutathione (GSH) is a reducing agent abundant in cells, especially in the cytosol, mitochondria, and nucleus. The GSH concentrations in blood and extracellular matrix are just one out of 100 to one out of 1000 of the intracellular concentration, respectively. This high redox potential difference caused by GSH, cysteine, and other reducing agents can break the reducible bonds, destabilize a lipid entity of the invention and result in the release of the payload. A disulfide bond can be used as the cleavable/reversible linker in a lipid entity of the invention, because it causes sensitivity to redox owing to the disulfide-to-thiol reduction reaction; a lipid entity of the invention can be made reduction sensitive by using two forms of a disulfide-conjugated multifunctional lipid where cleavage of the disulfide bond (e.g., via tris(2-carboxyethyl) phosphine, dithiothreitol, L-cysteine or GSH), can cause removal of the hydrophilic head group of the conjugate and alter the membrane organization leading to the release of the payload.

Enzymes can also be used as a trigger to release payload. Enzymes, including MMPs (e.g., MMP2), phospholipase A2, alkaline phosphatase, transglutaminase, or phosphatidylinositol-specific phospholipase C, have been found to be overexpressed in certain tissues, e.g., tumor tissues. In the presence of these enzymes, a specially engineered enzyme-sensitive lipid entity of the invention can be disrupted and release the payload. An MMP2-cleavable octapeptide (Gly-Pro-Leu-Gly-Ile-Ala-Gly-Gln) can be incorporated into a linker, and can have an antibody targeting moiety, e.g., antibody 2C5.

The invention also comprehends light- or energy-triggered delivery, e.g., the lipid entity of the invention can be light-sensitive, such that light or energy can facilitate structural and conformational changes, which lead to direct interaction of the lipid entity of the invention with the target cells via membrane fusion, photo-isomerism, photofragmentation or photopolymerization; such a moiety therefore can be a benzoporphyrin photosensitizer. Ultrasound can be a form of energy to trigger delivery; a lipid entity of the invention with a small quantity of a particular gas, including air or a perfluorated hydrocarbon, can be triggered to release with ultrasound, e.g., low-frequency ultrasound (LFUS). Magnetic delivery: A lipid entity of the invention can be magnetized by incorporation of magnetites, such as Fe₃O₄ or γ-Fe₂O₃, e.g., those that are less than 10 nm in size. Triggered delivery then occurs via exposure to a magnetic field.

### Cells and Organisms

Described in certain example embodiments herein is a cell or cell population containing one or more CREs of the present invention and/or one or more engineered polynucleotides and/or vectors described herein that comprises one or more CREs of the present invention. In an embodiment, one or more cells of an organism can contain one or more CREs of the present invention and/or one or more engineered polynucleotides and/or vectors described herein that comprises one or more CREs of the present invention. Such cells or organisms are also referred to herein as modified cells and modified organism, respectively. It will be appreciated that In an embodiment, the engineered polynucleotide of the present invention when expressed may result in a genetic, epigenetic, or other phenotypic change to a cell in which it is expressed. Such modified cells, even if the engineered polynucleotide is no longer present in the cell, are referred to as modified cells. To the extent that such modified cells are present in an organism, the organism can be referred to as a modified organism herein.

In an embodiment, the cell or cell population is a eukaryotic cell or cell population. In an embodiment, the eukaryotic cell or cell population is a mammalian cell or cell population. In an embodiment, the eukaryotic cell or cell population is a non-human mammalian cell or cell population. In an embodiment, the cell or cell population is a human cell or cell population. In an embodiment, the cell or cell population is a plant cell or cell population. In an embodiment, the cell or cell population is a fungal cell or cell population. In an embodiment, the cell or cell population is a prokaryotic cell or cell population. In an embodiment, the cell or cell population is part of an organism. In an embodiment, the organism is a non-human animal. In an embodiment, the organism is a human. In an embodiment, the cell or cell population is ex vivo or in vitro.

Exemplary non-human animal cell(s) are mammalian. Exemplary non-human mammals include, without limitation, non-human primates, canines, felines, swine, bovines, equines, ovines, camelids, ursids, leporids, murines, cricetids, cervids, giraffids, etc.

### Organisms

Also described herein are modified organisms. In an embodiment, the modified organisms can include one or more modified cells as are described elsewhere herein. In an embodiment, organisms are modified in a cell type, cell state, tissue type, specific manner. Without being bound by theory, this can be accomplished by use of the CREs of the present invention to regulate expression of a polynucleotide such that its expression or activity, and thus the modification, is restricted to a particular cell type, cell state, or tissue type. In an embodiment, the modified organism is a non-human mammal. In an embodiment, the modified organism is a modified plant. In an embodiment, the modified organism is an insect. In an embodiment, the modified organism is a fungus. In an embodiment, the modified organism is a fungus. Methods of making modified organisms are described in greater detail elsewhere herein.

The systems and methods described herein can be used in non-animal organisms, e.g., plants, fungi to generated modified non-animal organisms. The system and methods described can be used to generate non-human animal organisms. The system and methods described herein can be used to modify non-germline cells in a human. In an embodiment, the modification is expression of a polynucleotide of interest, gene of interest, and/or allele of interest.

The engineered polynucleotides and/or vectors can be introduced into plants and/or animals and/or cells thereof using any suitable delivery method and/or composition. Exemplary delivery method and/or compositions are described herein and will be appreciated by those of ordinary skill in the art in view of the description herein. Delivery of exogenous genes or modifying agents in the context of non-human animals has been previously demonstrated, such as in non-human primates, chickens (reviewed in Sid and Schusser et al 2018. Front. Genet. Doi.org/10.3389/fgene.2018.00456) and other avians (e.g. Scott et al. 2010. ILAR J. 51 (4): 353-361), cattle (Yum et al., 2016. Scientific Reports. 6:27185 and Tait-Burkard et al. 2018. Genome Biology. 19:2014.), sheep and goats (see e.g. Kalds et al., 2019. Front. Genet. Doi.org//10.3389/fgene.2019.00750), horses (see e.g. West and Gill. 2016. J. Equine Vet. Sci. 41:1-6), dogs (see e.g. D. Duan. Nature Biomedical Engineering. 2018. 2:795-796), reptiles (see e.g. Rasys et al. 2019. Cell Reports. 28:2288-2292), fish (including but not limited to zebrafish, see e.g. Datsomor et al. 2019. Scientific Reports. 9:7533, Liu et al. 2019. Front. Cell. Dev. Biol. doi.org/10.3389/fcell.2019.00013), insects (see e.g. Kotwica-Rolinska et al. 2019. Front. Physiol. doi.org/10.3389/fphys.2019.00891; Gantz and Akbari. 2018. Curr. Opin. Insect. Sci. 28:66-72), rabbits (see e.g. Kawano and Honda. 2017. Methods Mol. Biol. 4630:109-120; Liu et al., 2018. Nature Commun. 9:2717; and Liu et al. 2018. Gene. doi.org/10.1016/j.gene.2018.01.044), mice (see e.g. Hall et al. 2018. Curr Protoc Cell Biol. 81(1):e57), rats (see e.g. Back et al. 2019. Neuron. 102 (1): 105-119), amphibians (see e.g. Nakayama et al. 2013. Genesis. 51 (12): 835-843), nematodes (see e.g. J. B. Lok. 2019. Front. Genet. doi.org/10.3389/fgene.2019.00656), molluscs (see e.g. Abe and Kuroda. 2019. Development. 146: dev175976 doi: 10.1242/dev.175976, geckos, shrimp and other crustaceans (see e.g. Gui et al. Genes Genomes Genetics: 6 (11): 3757-3764), oysters (Yu et al. 2019; Mar. Biotechnol (NY) 21 (3): 301-309. doi: 10.1007/s10126-019-09885-y), and sponges (see e.g. Revilla-i-Domingo et al. 2018. Genetics. 210 (2) 435-443), the teachings of which can be adapted for use with one or more of the modifying agent(s) and/or systems described herein to generate a modified non-human animal or cell thereof.

In an embodiment, the cell or organism is a plant cell or plant or plant part. In general, the term “plant” refers to any photosynthetic, eukaryotic, unicellular, or multicellular organism of the kingdom Plantae characteristically growing by cell division, containing chloroplasts, and having cell walls comprised of cellulose. The term plant encompasses monocotyledonous and dicotyledonous plants. Specifically, the plants are intended to comprise without limitation angiosperm and gymnosperm plants such as acacia, alfalfa, amaranth, apple, apricot, artichoke, ash tree, asparagus, avocado, banana, barley, beans, beet, birch, beech, blackberry, blueberry, broccoli, Brussel's sprouts, cabbage, canola, cantaloupe, carrot, cassava, cauliflower, cedar, a cereal, celery, chestnut, cherry, Chinese cabbage, citrus, clementine, clover, coffee, corn, cotton, cowpea, cucumber, cypress, eggplant, elm, endive, eucalyptus, fennel, figs, fir, geranium, grape, grapefruit, groundnuts, ground cherry, gum hemlock, hickory, kale, kiwifruit, kohlrabi, larch, lettuce, leek, lemon, lime, locust, pine, maidenhair, maize, mango, maple, melon, millet, mushroom, mustard, nuts, oak, oats, oil palm, okra, onion, orange, an ornamental plant or flower or tree, papaya, palm, parsley, parsnip, pea, peach, peanut, pear, peat, pepper, persimmon, pigeon pea, pine, pineapple, plantain, plum, pomegranate, potato, pumpkin, radicchio, radish, rapeseed, raspberry, rice, rye, sorghum, safflower, sallow, soybean, spinach, spruce, squash, strawberry, sugar beet, sugarcane, sunflower, sweet potato, sweet corn, tangerine, tea, tobacco, tomato, trees, triticale, turf grasses, turnips, vine, walnut, watercress, watermelon, wheat, yams, yew, and zucchini. The term plant also encompasses Algae, which are mainly photoautotrophs unified primarily by their lack of roots, leaves, and other organelles that characterize higher plants. Exemplary plant cells include, without limitation, those cells of monocotyledonous and dicotyledonous plants, such as crops including grain crops (e.g., wheat, maize, rice, millet, barley), fruit crops (e.g., tomato, apple, pear, strawberry, orange), forage crops (e.g., alfalfa), root vegetable crops (e.g., carrot, potato, sugar beets, yam), leafy vegetable crops (e.g., lettuce, spinach); flowering plants (e.g., petunia, rose, chrysanthemum), conifers and pine trees (e.g., pine fir, spruce); plants used in phytoremediation (e.g., heavy metal accumulating plants); oil crops (e.g., sunflower, rape seed) and plants used for experimental purposes (e.g., Arabidopsis). Plant cells and tissues that can include the CREs and/or engineered polynucleotide compositions and/or systems of the present invention include, without limitation, roots, stems, leaves, flowers and reproductive structures, undifferentiated meristematic cells, parenchyma, collenchyma, sclerenchyma, xylem, phloem, epidermis, and germplasm. A part of a plant, e.g., a “plant tissue” may be treated according to the methods of the present invention to produce an improved plant. Plant tissue also encompasses plant cells. The term “plant cell” as used herein refers to individual units of a living plant, either in an intact whole plant or in an isolated form grown in in vitro tissue cultures, on media or agar, in suspension in a growth media or buffer or as a part of higher organized units, such as, for example, plant tissue, a plant organ, or a whole plant. A “protoplast” refers to a plant cell that has had its protective cell wall completely or partially removed using, for example, mechanical or enzymatic means resulting in an intact biochemical competent unit of living plant that can reform their cell wall, proliferate, and regenerate into a whole plant under proper growing conditions. This also includes the progeny of plant cells that include one or more of the CREs of the present invention, engineered polynucleotides, and other gene products, compositions and/or systems of the present invention, such as the progeny of a transgenic plant, is one that is born of, begotten by, or derived from a plant to which composition and/or system of the present invention is delivered.

Thus, it will be appreciated that compositions and/or systems of the present invention can be used over a broad range of plants, such as for example with dicotyledonous plants belonging to the orders Magniolales, Illiciales, Laurales, Piperales, Aristochiales, Nymphaeales, Ranunculales, Papeverales, Sarraceniaceae, Trochodendrales, Hamamelidales, Eucomiales, Leitneriales, Myricales, Fagales, Casuarinales, Caryophyllales, Batales, Polygonales, Plumbaginales, Dilleniales, Theales, Malvales, Urticales, Lecythidales, Violales, Salicales, Capparales, Ericales, Diapensales, Ebenales, Primulales, Rosales, Fabales, Podostemales, Haloragales, Myrtales, Cornales, Proteales, San tales, Rafflesiales, Celastrales, Euphorbiales, Rhamnales, Sapindales, Juglandales, Geraniales, Polygalales, Umbellales, Gentianales, Polemoniales, Lamiales, Plantaginales, Scrophulariales, Campanulales, Rubiales, Dipsacales, and Asterales; monocotyledonous plants such as those belonging to the orders Alismatales, Hydrocharitales, Najadales, Triuridales, Commelinales, Eriocaulales, Restionales, Poales, Juncales, Cyperales, Typhales, Bromeliales, Zingiberales, Arecales, Cyclanthales, Pandanales, Arales, Lilliales, and Orchid ales, or with plants belonging to Gymnospermae, e.g., those belonging to the orders Pinales, Ginkgoales, Cycadales, Araucariales, Cupressales and Gnetales. It will also be appreciated that the compositions and/or systems of the present invention can be used over a broad range of plant species, included in the non-limitative list of dicot, monocot or gymnosperm genera hereunder: Atropa, Alseodaphne, Anacardium, Arachis, Beilschmiedia, Brassica, Carthamus, Cocculus, Croton, Cucumis, Citrus, Citrullus, Capsicum, Catharanthus, Cocos, Coffea, Cucurbita, Daucus, Duguetia, Eschscholzia, Ficus, Fragaria, Glaucium, Glycine, Gossypium, Helianthus, Hevea, Hyoscyamus, Lactuca, Landolphia, Linum, Litsea, Lycopersicon, Lupinus, Manihot, Majorana, Malus, Medicago, Nicotiana, Olea, Parthenium, Papaver, Persea, Phaseolus, Pistacia, Pisum, Pyrus, Prunus, Raphanus, Ricinus, Senecio, Sinomenium, Stephania, Sinapis, Solanum, Theobroma, Trifolium, Trigonella, Vicia, Vinca, Vilis, and Vigna; and the genera Allium, Andropogon, Aragrostis, Asparagus, Avena, Cynodon, Elaeis, Festuca, Festulolium, Heterocallis, Hordeum, Lemna, Lolium, Musa, Oryza, Panicum, Pannesetum, Phleum, Poa, Secale, Sorghum, Triticum, Zea, Abies, Cunninghamia, Ephedra, Picea, Pinus, and Pseudotsuga.

It will also be appreciated that the compositions and/or systems of the present invention can be used over a broad range of “algae” or “algae cells”; including for example algae selected from several eukaryotic phyla, including the Rhodophyta (red algae), Chlorophyta (green algae), Phaeophyta (brown algae), Bacillariophyta (diatoms), Eustigmatophyta and dinoflagellates as well as the prokaryotic phylum Cyanobacteria (blue-green algae). The term “algae” includes for example algae selected from: Amphora, Anabaena, Anikstrodesmis, Botryococcus, Chaetoceros, Chlamydomonas, Chlorella, Chlorococcum, Cyclotella, Cylindrotheca, Dunaliella, Emiliana, Euglena, Hematococcus, Isochrysis, Monochrysis, Monoraphidium, Nannochloris, Nannnochloropsis, Navicula, Nephrochloris, Nephroselmis, Nitzschia, Nodularia, Nostoc, Oochromonas, Oocystis, Oscillartoria, Pavlova, Phaeodactylum, Playtmonas, Pleurochrysis, Porhyra, Pseudoanabaena, Pyramimonas, Stichococcus, Synechococcus, Synechocystis, Tetraselmis, Thalassiosira, and Trichodesmium.

A part of a plant, e.g., a “plant tissue” may be treated according to the methods of the present invention to produce an improved plant. Plant tissue also encompasses plant cells. The term “plant cell” as used herein refers to individual units of a living plant, either in an intact whole plant or in an isolated form grown in in vitro tissue cultures, on media or agar, in suspension in a growth media or buffer or as a part of higher organized unites, such as, for example, plant tissue, a plant organ, or a whole plant.

A “protoplast” refers to a plant cell that has had its protective cell wall completely or partially removed using, for example, mechanical or enzymatic means resulting in an intact biochemical competent unit of living plant that can reform their cell wall, proliferate and regenerate grow into a whole plant under proper growing conditions.

The term “transformation” broadly refers to the process by which a plant host is genetically modified by the introduction of DNA by means of Agrobacteria or one of a variety of chemical or physical methods. As used herein, the term “plant host” refers to plants, including any cells, tissues, organs, or progeny of the plants. Many suitable plant tissues or plant cells can be transformed and include, but are not limited to, protoplasts, somatic embryos, pollen, leaves, seedlings, stems, calli, stolons, microtubers, and shoots. A plant tissue also refers to any clone of such a plant, seed, progeny, propagule whether generated sexually or asexually, and descendants of any of these, such as cuttings or seed.

The term “transformed” as used herein, refers to a cell, tissue, organ, or organism into which a foreign DNA molecule, such as a construct, has been introduced. The introduced DNA molecule may be integrated into the genomic DNA of the recipient cell, tissue, organ, or organism such that the introduced DNA molecule is transmitted to the subsequent progeny. In these embodiments, the “transformed” or “transgenic” cell or plant may also include progeny of the cell or plant and progeny produced from a breeding program employing such a transformed plant as a parent in a cross and exhibiting an altered phenotype resulting from the presence of the introduced DNA molecule. Preferably, the transgenic plant is fertile and capable of transmitting the introduced DNA to progeny through sexual reproduction.

The term “progeny”, such as the progeny of a transgenic plant, is one that is born of, begotten by, or derived from a plant or the transgenic plant. The introduced DNA molecule may also be transiently introduced into the recipient cell such that the introduced DNA molecule is not inherited by subsequent progeny and thus not considered “transgenic”. Accordingly, as used herein, a “non-transgenic” plant or plant cell is a plant which does not contain a foreign DNA stably integrated into its genome.

The term “plant promoter” as used herein is a promoter capable of initiating transcription in plant cells, whether or not its origin is a plant cell. Exemplary suitable plant promoters include, but are not limited to, those that are obtained from plants, plant viruses, and bacteria such as Agrobacterium or Rhizobium which comprise genes expressed in plant cells.

As used herein, the term “yeast cell” refers to any fungal cell within the phyla Ascomycota and Basidiomycota. Yeast cells may include budding yeast cells, fission yeast cells, and mold cells. Without being limited to these organisms, many types of yeast used in laboratory and industrial settings are part of the phylum Ascomycota. In an embodiment, the yeast cell is an S. cerevisiae, Kluyveromyces marxianus, or Issatchenkia orientalis cell. Other yeast cells may include without limitation Candida spp. (e.g., Candida albicans), Yarrowia spp. (e.g., Yarrowia lipolytica), Pichia spp. (e.g., Pichia pastoris), Kluyveromyces spp. (e.g., Kluyveromyces lactis and Kluyveromyces marxianus), Neurospora spp. (e.g., Neurospora crassa), Fusarium spp. (e.g., Fusarium oxysporum), and Issatchenkia spp. (e.g., Issatchenkia orientalis, a.k.a. Pichia kudriavzevii and Candida acidothermophilum). In an embodiment, the fungal cell is a filamentous fungal cell. As used herein, the term “filamentous fungal cell” refers to any type of fungal cell that grows in filaments, i.e., hyphae or mycelia. Examples of filamentous fungal cells may include without limitation Aspergillus spp. (e.g., Aspergillus niger), Trichoderma spp. (e.g., Trichoderma reesei), Rhizopus spp. (e.g., Rhizopus oryzae), and Mortierella spp. (e.g., Mortierella isabellina).

In an embodiment, the fungal cell is an industrial strain. As used herein, “industrial strain” refers to any strain of fungal cell used in or isolated from an industrial process, e.g., production of a product on a commercial or industrial scale. Industrial strain may refer to a fungal species that is typically used in an industrial process, or it may refer to an isolate of a fungal species that may be also used for non-industrial purposes (e.g., laboratory research). Examples of industrial processes may include fermentation (e.g., in production of food or beverage products), distillation, biofuel production, production of a compound, and production of a polypeptide. Examples of industrial strains may include, without limitation, JAY270 and ATCC4124.

In an embodiment, the fungal cell is a polyploid cell. As used herein, a “polyploid” cell may refer to any cell whose genome is present in more than one copy. A polyploid cell may refer to a type of cell that is naturally found in a polyploid state, or it may refer to a cell that has been induced to exist in a polyploid state (e.g., through specific regulation, alteration, inactivation, activation, or modification of meiosis, cytokinesis, or DNA replication). A polyploid cell may refer to a cell whose entire genome is polyploid, or it may refer to a cell that is polyploid in a particular genomic locus of interest.

In an embodiment, the fungal cell is a diploid cell. As used herein, a “diploid” cell may refer to any cell whose genome is present in two copies. A diploid cell may refer to a type of cell that is naturally found in a diploid state, or it may refer to a cell that has been induced to exist in a diploid state (e.g., through specific regulation, alteration, inactivation, activation, or modification of meiosis, cytokinesis, or DNA replication). For example, the S. cerevisiae strain S228C may be maintained in a haploid or diploid state. A diploid cell may refer to a cell whose entire genome is diploid, or it may refer to a cell that is diploid in a particular genomic locus of interest. In an embodiment, the fungal cell is a haploid cell. As used herein, a “haploid” cell may refer to any cell whose genome is present in one copy. A haploid cell may refer to a type of cell that is naturally found in a haploid state, or it may refer to a cell that has been induced to exist in a haploid state (e.g., through specific regulation, alteration, inactivation, activation, or modification of meiosis, cytokinesis, or DNA replication). For example, the S. cerevisiae strain S228C may be maintained in a haploid or diploid state. A haploid cell may refer to a cell whose entire genome is haploid, or it may refer to a cell that is haploid in a particular genomic locus of interest.

In an embodiment, are plants and/or plant cells and/or animal, in particular a non-human animal, that can be produced by one or more of the methods described herein, or a progeny thereof. The progeny may be a clone of the produced plant or animal, or may result from sexual reproduction by crossing with other individuals of the same species to introgress further desirable traits into their offspring. The cell may be in vivo or ex vivo in the cases of multicellular organisms, particularly plants, animals and more particularly non-human animals. This is described in greater detail herein.

### Pharmaceutical Formulations

Also described herein are pharmaceutical formulations that can contain an amount, effective amount, and/or least effective amount, and/or therapeutically effective amount of one or more compounds, molecules, compositions, systems, vectors, vector systems, systems, cells, or any combination thereof of the present invention, which are also referred to as the primary active agent or ingredient, and a pharmaceutically acceptable carrier or excipient. As used herein, “pharmaceutical formulation” refers to the combination of an active agent, compound, or ingredient with a pharmaceutically acceptable carrier or excipient, making the composition suitable for diagnostic, therapeutic, or preventive use in vitro, in vivo, or ex vivo. As used herein, “pharmaceutically acceptable carrier or excipient” refers to a carrier or excipient that is useful in preparing a pharmaceutical formulation that is generally safe, non-toxic, and is neither biologically or otherwise undesirable, and includes a carrier or excipient that is acceptable for veterinary use as well as human pharmaceutical use. A “pharmaceutically acceptable carrier or excipient” as used in the specification and claims includes both one and more than one such carrier or excipient. When present, a compound or composition can optionally be present in the pharmaceutical formulation as a pharmaceutically acceptable salt.

In an embodiment, the active ingredient is present as a pharmaceutically acceptable salt of the active ingredient. As used herein, “pharmaceutically acceptable salt” refers to any acid or base addition salt whose counter-ions are non-toxic to the subject to which they are administered in pharmaceutical doses of the salts. Suitable salts include hydrobromide, iodide, nitrate, bisulfate, phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, camphorsulfonate, napthalenesulfonate, propionate, malonate, mandelate, malate, phthalate, and pamoate.

The pharmaceutical formulations described herein can be administered to a subject in need thereof via any suitable method or route. Suitable administration routes can include, but are not limited to auricular (otic), buccal, conjunctival, cutaneous, dental, electro-osmosis, endocervical, endosinusial, endotracheal, enteral, epidural, extra-amniotic, extracorporeal, hemodialysis, infiltration, interstitial, intra-abdominal, intra-amniotic, intra-arterial, intra-articular, intrabiliary, intrabronchial, intrabursal, intracardiac, intracartilaginous, intracaudal, intracavernous, intracavitary, intracerebral, intracisternal, intracorneal, intracoronal (dental), intracoronary, intracorporus cavernosum, intradermal, intradiscal, intraductal, intraduodenal, intradural, intraepidermal, intraesophageal, intragastric, intragingival, intraileal, intralesional, intraluminal, intralymphatic, intramedullary, intrameningeal, intramuscular, intraocular, intraovarian, intrapericardial, intraperitoneal, intrapleural, intraprostatic, intrapulmonary, intrasinal, intraspinal, intrasynovial, intratendinous, intratesticular, intrathecal, intrathoracic, intratubular, intratumor, intratympanic, intrauterine, intravascular, intravenous, intravenous bolus, intravenous drip, intraventricular, intravesical, intravitreal, iontophoresis, irrigation, laryngeal, nasal, nasogastric, occlusive dressing technique, ophthalmic, oral, oropharyngeal, other, parenteral, percutaneous, periarticular, peridural, perineural, periodontal, rectal, respiratory (inhalation), retrobulbar, soft tissue, subarachnoid, subconjunctival, subcutaneous, sublingual, submucosal, topical, transdermal, transmucosal, transplacental, transtracheal, transtympanic, ureteral, urethral, and/or vaginal administration, and/or any combination of the above administration routes, which typically depends on the disease to be treated and/or the active ingredient(s).

Where appropriate, the primary and/or additional active agent compounds, molecules, compositions, vectors, vector systems, systems, cells, or any combination thereof of the present invention can be provided to a subject in need thereof as an ingredient, such as an active ingredient or agent, in a pharmaceutical formulation. As such, also described are pharmaceutical formulations containing one or more of the compounds and salts thereof, or pharmaceutically acceptable salts thereof described herein.

In an embodiment, the gene product under control of one or more CREs of the present invention to be delivered is a replacement protein therapy or genetic modifying system. In an embodiment, the subject has a disease or disorder to be treated with a CRISPR-Cas system or other genetic modifying system or replacement gene or gene product therapy, such as a genetic disease or disorder. Without being bound by theory, it can be desirable to spatially control the activity of the genetic modifying system, gene, or protein therapy, or the amount of genetic modifying system or gene or protein therapy. Without being bound by theory, such control can be achieved In an embodiment, by the particular one or more CREs used to regulate expression of the polynucleotide encoding the genetic modifying system or component thereof, gene therapy and/or protein therapy. As used herein, “agent” refers to any substance, compound, molecule, and the like, which can be biologically active or otherwise can induce a biological and/or physiological effect on a subject to which it is administered to. As used herein, “active agent” or “active ingredient” refers to a substance, compound, or molecule, which is biologically active or otherwise, induces a biological or physiological effect on a subject to which it is administered to. In other words, “active agent” or “active ingredient” refers to a component or components of a composition to which the whole or part of the effect of the composition is attributed. An agent can be a primary active agent, or in other words, the component(s) of a composition to which the whole or part of the effect of the composition is attributed. An agent can be a secondary agent, or in other words, the component(s) of a composition to which an additional part and/or other effect of the composition is attributed.

### Pharmaceutically Acceptable Carriers and Secondary Ingredients and Agents

The pharmaceutical formulation can include a pharmaceutically acceptable carrier. Suitable pharmaceutically acceptable carriers include, but are not limited to water, salt solutions, alcohols, gum arabic, vegetable oils, benzyl alcohols, polyethylene glycols, gelatin, carbohydrates (such as lactose, amylose, or starch), magnesium stearate, talc, silicic acid, viscous paraffin, perfume oil, fatty acid esters, hydroxy methylcellulose, and polyvinyl pyrrolidone, which do not deleteriously react with the active composition.

The pharmaceutical formulations can be sterilized, and if desired, mixed with agents, such as lubricants, preservatives, stabilizers, wetting agents, emulsifiers, salts for influencing osmotic pressure, buffers, coloring, flavoring and/or aromatic substances, and the like which do not deleteriously react with the active compound.

In an embodiment, the pharmaceutical formulation can also include an effective amount of secondary active agents, including but not limited to, biological agents or molecules including, but not limited to, e.g. polynucleotides, amino acids, peptides, polypeptides, antibodies, aptamers, ribozymes, hormones, immunomodulators, antipyretics, anxiolytics, antipsychotics, analgesics, antispasmodics, anti-inflammatories, anti-histamines, anti-infectives, chemotherapeutics, nucleic acid modification systems (e.g. CRISPR-Cas systems), and any combination thereof.

In an embodiment, the secondary agent included in the formulation is a performance modifier. In this context, a “performance modifier” is a compound, composition, or other ingredient that modifies the function and/or activity level of a primary or other secondary active agent. In an embodiment, the performance modifier is an Anti-CRISPR molecule (Acr) (see e.g., Marino et al., Nat. Methods. 2020. 17 (5): 471-479). In an embodiment, the performance modifier is an anti-anti-CRISPR molecule, which is effective to regulate or otherwise modify the activity of a CRISPR-Cas gene product, including but not limited to Acas (see e.g., Stanley et al., Cell. 178 (6): 1452-1464.e13 (2019)) and small molecules (see e.g., Nakamura et al., Nat. Comm. 10, Article number: 194 (2019)).

### Effective Amounts

In an embodiment, the amount of the primary active agent and/or optional secondary agent can be an effective amount, least effective amount, and/or therapeutically effective amount. As used herein, “effective amount” refers to the amount of the primary and/or optional secondary agent included in the pharmaceutical formulation that achieves one or more desired effects. As used herein, “least effective” amount refers to the lowest amount of the primary and/or optional secondary agent that achieves one or more therapeutic or other desired effects. As used herein, “therapeutically effective amount” refers to the amount of the primary and/or optional secondary agent included in the pharmaceutical formulation that achieves one or more therapeutic effects. In an embodiment, the therapeutic effects include, but are not limited, genome modification (e.g., insertion, deletion, substitution, mutation, and/or the like of one or more polynucleotides), epigenome modification, reporter gene expression, exogenous or replacement gene expression, killing or inhibiting the growth of a cell, promoting cell growth and/or differentiation, and/or the like.

The effective amount, least effective amount, and/or therapeutically effective amount of the primary and optional secondary active agent described elsewhere herein contained in the pharmaceutical formulation can be any non-zero amount ranging from about 0 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 pg, ng, μg, mg, or g or be any numerical value or subrange within any of these ranges.

In an embodiment, the effective amount, least effective amount, and/or therapeutically effective amount can be an effective concentration, least effective concentration, and/or therapeutically effective concentration, which can each be any non-zero amount ranging from about 0 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 pM, nM, μM, mM, or M or be any numerical value or subrange within any of these ranges. Similar to effective amount, least effective amount, and therapeutic effective amount, effective concentration, least effective concentration, and/or therapeutically effective concentration is the concentration where a desired effect is achieved, the least concentration at which a desired effect or effects are achieved, or the concentration at which one or more therapeutic effects are achieved, respectively. Exemplary effects and/or therapeutic effects are described in greater detail elsewhere herein.

In other embodiments, the effective amount, least effective amount, and/or therapeutically effective amount of the primary and optional secondary active agent can be any non-zero amount ranging from about 0 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 international units (IU) or be any numerical value or subrange within any of these ranges.

In an embodiment, the primary and/or the optional secondary active agent present in the pharmaceutical formulation can be any non-zero amount ranging from about 0 to 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, 99.9% w/w, v/v, or w/v of the pharmaceutical formulation or be any numerical value or subrange within any of these ranges.

In an embodiment where a cell or cell population is present in the pharmaceutical formulation (e.g., as a primary and/or secondary active agent), the effective amount of cells can be any amount ranging from about 1 or 2 cells to 1×10¹ cells/mL, 1×10²⁰ cells/mL or more, such as about 1×10¹ cells/mL, 1×10² cells/mL, 1×10³ cells/mL, 1×10⁴ cells/mL, 1×10⁵ cells/mL, 1×10⁶ cells/mL, 1×10⁷ s/mL, 1×10⁸ cells/mL, 1×10⁹ cells/mL, 1×10¹⁰ cells/mL, 1×10¹¹ cells/mL, 1×10¹² cells/mL, 1×10¹³ cells/mL, 1×10¹⁴ cells/mL, 1×10¹⁵ cells/mL, 1×10¹⁶ cells/mL, 1×10¹⁷ cells/mL, 1×10¹⁸ cells/mL, 1×10¹⁹ cells/mL, to/or about 1×10²⁰/cells mL or any numerical value or subrange within any of these ranges.

In an embodiment, the amount or effective amount, particularly where an infective particle is being delivered (e.g., a virus particle having the primary or secondary agent as a cargo), the effective amount of virus particles can be expressed as a titer (plaque forming units per unit of volume) or as a MOI (multiplicity of infection). In an embodiment, the effective amount can be about 1×10¹ particles per pL, nL, μL, mL, or L to 1×10²⁰ particles per pL, nL, μL, mL, or L or more, such as about 1×10¹, 1×10², 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, 1×10¹⁶, 1×10¹⁷, 1×10¹⁸, 1×10¹⁹, to/or about 1×10²⁰ particles per pL, nL, μL, mL, or L. In an embodiment, the effective titer can be about 1×10¹ transforming units per pL, nL, μL, mL, or L to 1×10²⁰ transforming units per pL, nL, μL, mL, or L or more, such as about 1×10¹, 1×10², 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, 1×10¹⁶, 1×10¹⁷, 1×10¹⁸, 1×10¹⁹, to/or about 1×10²⁰ transforming units per pL, nL, μL, mL, or L or any numerical value or subrange within these ranges. In an embodiment, the MOI of the pharmaceutical formulation can range from about 0.1 to 10 or more, such as 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10 or more or any numerical value or subrange within these ranges.

In an embodiment, the amount or effective amount of one or more of the active agent(s) described herein contained in the pharmaceutical formulation can range from about 1 μg/kg to about 10 mg/kg based upon the bodyweight of the subject in need thereof or average bodyweight of the specific patient population to which the pharmaceutical formulation can be administered.

In embodiments where there is a secondary agent contained in the pharmaceutical formulation, the effective amount of the secondary active agent will vary depending on the secondary agent, the primary agent, the administration route, subject age, disease, stage of disease, among other things, which can be appreciated by one of ordinary skill in the art.

When optionally present in the pharmaceutical formulation, the secondary active agent can be included in the pharmaceutical formulation or can exist as a stand-alone compound or pharmaceutical formulation that can be administered contemporaneously or sequentially (e.g., before or after with the compound, derivative thereof, or pharmaceutical formulation thereof.

In an embodiment, the effective amount of the secondary active agent, when optionally present, is any non-zero amount ranging from about 0 to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99% w/w, v/v, or w/v of the total active agents present in the pharmaceutical formulation or any numerical value or subrange within these ranges. In additional embodiments, the effective amount of the secondary active agent is any non-zero amount ranging from about 0 to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99% w/w, v/v, or w/v of the total pharmaceutical formulation or any numerical value or subrange within these ranges.

### Dosage Forms

In an embodiment, the pharmaceutical formulations described herein can be provided in a dosage form. The dosage form can be administered to a subject in need thereof. The dosage form can be effective to generate a specific concentration, such as an effective concentration, at a given site in the subject in need thereof. As used herein, “dose,” “unit dose,” or “dosage” can refer to physically discrete units suitable for use in a subject, each unit containing a predetermined quantity of the primary active agent, and optionally present secondary active ingredient, and/or a pharmaceutical formulation thereof calculated to produce the desired response or responses in association with its administration. In an embodiment, the given site is proximal to the administration site. In an embodiment, the given site is distal to the administration site. In some cases, the dosage form contains a greater amount of one or more of the active ingredients present in the pharmaceutical formulation than the final intended amount needed to reach a specific region or location within the subject to account for loss of the active components such as via first and second pass metabolism.

The dosage forms can be adapted for administration by any appropriate route. Appropriate routes include, but are not limited to, oral (including buccal or sublingual), rectal, intraocular, inhaled, intranasal, topical (including buccal, sublingual, or transdermal), vaginal, parenteral, subcutaneous, intramuscular, intravenous, internasal, and intradermal. Other appropriate routes are described elsewhere herein. Such formulations can be prepared by any method known in the art.

Dosage forms adapted for oral administration can be discrete dosage units such as capsules, pellets or tablets, powders or granules, solutions, or suspensions in aqueous or non-aqueous liquids; edible foams or whips, or in oil-in-water liquid emulsions or water-in-oil liquid emulsions. In an embodiment, the pharmaceutical formulations adapted for oral administration also include one or more agents which flavor, preserve, color, or help disperse the pharmaceutical formulation. Dosage forms prepared for oral administration can also be in the form of a liquid solution that can be delivered as a foam, spray, or liquid solution. The oral dosage form can be administered to a subject in need thereof. Where appropriate, the dosage forms described herein can be microencapsulated.

The dosage form can also be prepared to prolong or sustain the release of any ingredient. In an embodiment, compounds, molecules, compositions, vectors, vector systems, cells, or a combination thereof described herein can be the ingredient whose release is delayed. In an embodiment, the primary active agent is the ingredient whose release is delayed. In an embodiment, an optional secondary agent can be the ingredient whose release is delayed. Suitable methods for delaying the release of an ingredient include, but are not limited to, coating or embedding the ingredients in materials, such as polymers, wax, gels, and the like. Delayed release dosage formulations can be prepared as described in standard references such as “Pharmaceutical dosage form tablets,” eds. Liberman et. al. (New York, Marcel Dekker, Inc., 1989), “Remington—The science and practice of pharmacy”, 20^{th} ed., Lippincott Williams & Wilkins, Baltimore, MD, 2000, and “Pharmaceutical dosage forms and drug delivery systems”, 6^{th} Edition, Ansel et al., (Media, PA: Williams and Wilkins, 1995). These references provide information on excipients, materials, equipment, and processes for preparing tablets and capsules and delayed release dosage forms of tablets and pellets, capsules, and granules. The delayed release can be anywhere from about an hour to about 3 months or more.

Examples of suitable coating materials to prolong the release of an ingredient include, but are not limited to, cellulose polymers such as cellulose acetate phthalate, hydroxypropyl cellulose, hydroxypropyl methylcellulose, hydroxypropyl methylcellulose phthalate, and hydroxypropyl methylcellulose acetate succinate; polyvinyl acetate phthalate, acrylic acid polymers and copolymers, and methacrylic resins that are commercially available under the trade name EUDRAGIT® (Roth Pharma, Westerstadt, Germany), zein, shellac, and polysaccharides.

Coatings may be formed with a different ratio of water-soluble polymers, water-insoluble polymers, and/or pH-dependent polymers, with or without water-insoluble/water-soluble non-polymeric excipients, to produce the desired release profile. The coating is either performed on the dosage form (matrix or simple) which includes, but is not limited to, tablets (compressed with or without coated beads), capsules (with or without coated beads), beads, particle compositions, “ingredient as is” formulated as, but is not limited to, a suspension form or as a sprinkle dosage form.

Where appropriate, the dosage forms described herein can be a liposome. In these embodiments, primary active ingredient(s), and/or optional secondary active ingredient(s), and/or pharmaceutically acceptable salt thereof where appropriate are incorporated into a liposome. In embodiments where the dosage form is a liposome, the pharmaceutical formulation is thus a liposomal formulation. The liposomal formulation can be administered to a subject in need thereof.

Dosage forms adapted for topical administration can be formulated as ointments, creams, suspensions, lotions, powders, solutions, pastes, gels, sprays, aerosols, or oils. In an embodiment for treatments of the eye or other external tissues, for example, the mouth or the skin, the pharmaceutical formulations are applied as a topical ointment or cream. When formulated in an ointment, a primary active ingredient, optional secondary active ingredient, and/or pharmaceutically acceptable salt thereof where appropriate can be formulated with a paraffinic or water-miscible ointment base. In other embodiments, the primary and/or secondary active ingredient can be formulated in a cream with an oil-in-water cream base or a water-in-oil base. Dosage forms adapted for topical administration in the mouth include lozenges, pastilles, and mouth washes.

Dosage forms adapted for nasal or inhalation administration include aerosols, solutions, suspension drops, gels, or dry powders. In an embodiment, a primary active ingredient, optional secondary active ingredient, and/or pharmaceutically acceptable salt thereof where appropriate can be in a dosage form adapted for inhalation and is in a particle-size-reduced form that is obtained or obtainable by micronization. In an embodiment, the particle size of the size reduced (e.g., micronized) compound or salt or solvate thereof, is defined by a D₅₀ value of about 0.5 to about 10 microns as measured by an appropriate method known in the art. Dosage forms adapted for administration by inhalation also include particle dusts or mists. Suitable dosage forms wherein the carrier or excipient is a liquid for administration as a nasal spray or drops include aqueous or oil solutions/suspensions of an active (primary and/or secondary) ingredient, which may be generated by various types of metered dose pressurized aerosols, nebulizers, or insufflators. The nasal/inhalation formulations can be administered to a subject in need thereof.

In an embodiment, the dosage forms are aerosol formulations suitable for administration by inhalation. In some of these embodiments, the aerosol formulation contains a solution or fine suspension of a primary active ingredient, secondary active ingredient, and/or pharmaceutically acceptable salt thereof where appropriate and a pharmaceutically acceptable aqueous or non-aqueous solvent. Aerosol formulations can be presented in single or multi-dose quantities in sterile form in a sealed container. For some of these embodiments, the sealed container is a single-dose or multi-dose nasal or an aerosol dispenser fitted with a metering valve (e.g., metered dose inhaler), which is intended for disposal once the contents of the container have been exhausted.

Where the aerosol dosage form is contained in an aerosol dispenser, the dispenser contains a suitable propellant under pressure, such as compressed air, carbon dioxide, or an organic propellant, including but not limited to a hydrofluorocarbon. The aerosol formulation dosage forms in other embodiments are contained in a pump-atomizer. The pressurized aerosol formulation can also contain a solution or a suspension of a primary active ingredient, optional secondary active ingredient, and/or pharmaceutically acceptable salt thereof. In further embodiments, the aerosol formulation also contains co-solvents and/or modifiers incorporated to improve, for example, the stability and/or taste and/or fine particle mass characteristics (amount and/or profile) of the formulation. Administration of the aerosol formulation can be once daily or several times daily, for example, 2, 3, 4, or 8 times daily, in which 1, 2, 3, or more doses are delivered each time. The aerosol formulations can be administered to a subject in need thereof.

For some dosage forms suitable and/or adapted for inhaled administration, the pharmaceutical formulation is a dry powder inhalable formulation. In addition to a primary active agent, optional secondary active ingredient, and/or pharmaceutically acceptable salt thereof where appropriate, such a dosage form can contain a powder base such as lactose, glucose, trehalose, mannitol, and/or starch. In some of these embodiments, a primary active agent, secondary active ingredient, and/or pharmaceutically acceptable salt thereof where appropriate is in a particle-size reduced form.

In further embodiments, a performance modifier, such as L-leucine or another amino acid, cellobiose octaacetate, and/or metal salts of stearic acid, such as magnesium or calcium stearate. In an embodiment, the aerosol formulations are arranged so that each metered dose of aerosol contains a predetermined amount of an active ingredient, such as the one or more of the compositions, compounds, vector(s), molecules, cells, and combinations thereof described herein.

Dosage forms adapted for vaginal administration can be presented as pessaries, tampons, creams, gels, pastes, foams, or spray formulations. Dosage forms adapted for rectal administration include suppositories or enemas. The vaginal formulations can be administered to a subject in need thereof.

Dosage forms adapted for parenteral administration and/or adapted for injection can include aqueous and/or non-aqueous sterile injection solutions, which can contain antioxidants, buffers, bacteriostats, solutes that render the composition isotonic with the blood of the subject, and aqueous and non-aqueous sterile suspensions, which can include suspending agents and thickening agents. The dosage forms adapted for parenteral administration can be presented in single-unit dose or multi-unit dose containers, including but not limited to sealed ampoules or vials. The doses can be lyophilized and re-suspended in a sterile carrier to reconstitute the dose prior to administration. Extemporaneous injection solutions and suspensions can be prepared In an embodiment, from sterile powders, granules, and tablets. The parenteral formulations can be administered to a subject in need thereof.

For some embodiments, the dosage form contains a predetermined amount of a primary active agent, secondary active ingredient, and/or pharmaceutically acceptable salt thereof where appropriate per unit dose. In an embodiment, the predetermined amount of primary active agent, secondary active ingredient, and/or pharmaceutically acceptable salt thereof where appropriate can be an effective amount, a least effective amount, and/or a therapeutically effective amount. In other embodiments, the predetermined amount of a primary active agent, secondary active agent, and/or pharmaceutically acceptable salt thereof where appropriate, can be an appropriate fraction of the effective amount of the active ingredient.

### Co-Therapies and Combination Therapies

In an embodiment, the pharmaceutical formulation(s) described herein are part of a combination treatment or combination therapy. The combination treatment can include the pharmaceutical formulation described herein and an additional treatment modality. The additional treatment modality can be a chemotherapeutic, a genetic modifier, a biological therapeutic, surgery, radiation, diet modulation, environmental modulation, a physical activity modulation, and combinations thereof.

In an embodiment, the co-therapy or combination therapy additionally includes but is not limited to, polynucleotides, amino acids, peptides, polypeptides, antibodies, aptamers, ribozymes, hormones, immunomodulators, antipyretics, anxiolytics, antipsychotics, analgesics, antispasmodics, anti-inflammatories anti-histamines, anti-infectives, chemotherapeutics, genetic modifiers (e.g., CRISPR-Cas systems), and combinations thereof.

### Devices

Described in certain example embodiment herein are devices configured to detect a specific cell type, cell state, tissue type, and/or environment of one or more cells comprising an engineered reporter polynucleotide described in greater detail elsewhere herein, a vector comprising the same, and/or a delivery vehicle comprising the same. In an embodiment, the device comprises microfluidic device, a lateral flow device, a tangential flow device, a normal flow device, a micro-electromechanical system, or any combination thereof. In an embodiment, the device further comprises one or more reagents, including but not limited to detection reagents, wherein the detection reagent comprises a sequence-specific binding molecule or system capable of specifically binding the reporter polynucleotide, optionally at the target sequence for a sequence-specific binding molecule or system. In an embodiment, the sequence-specific binding molecule or system comprises a programmable nuclease or system thereof, optionally wherein the programmable nuclease or system thereof is a Cas or Cas-based system, or an OMEGA system.

In general, the devices can be configured to receive a sample that is composed of one or more cells. Before or after receiving the sample, an engineered reporter polynucleotide is delivered to the one or more cells. Expression or inhibition of the reporter is limited to the particular cell type, state, tissue type, or environment in which the one or more CREs are active in. Detection of a signal produced by the report can occur in the device. The device can be configured to provide an output based on signal detection, which can be direct visible detection of a signal or other output that provides signal information to a user.

### Diagnostic Devices

The assays or component thereof can be carried out on a device, such as tube, capillary, lateral flow strip, chip, cartridge, or another device. The systems and/or assays described herein can be embodied on diagnostic devices. Devices can include very simple devices such as tubes for containing a single sample that contains all the reagents necessary, all within the single tube, to carry out an engineered reporter polynucleotide detection reaction: delivery, e.g., to a cell or a population of cells, of an engineered reporter polynucleotide (e.g., a reporter polynucleotide operatively coupled to an engineered cis-regulator element (CRE), or a delivery system comprising the same) as described herein, expression of the same in the cell or the population of cells, and production of a detectable signal (such as a colometric, turbidity shift, or fluorescent signal). Other devices can be complex fully automated devices that are capable of handling tens to thousands of samples at time. As is described in greater detail elsewhere herein, one or more engineered reporter polynucleotide detection systems (e.g., one or more compositions required to perform the engineered reporter polynucleotide detection reaction) can be included in the device (e.g., sample preparation reagents (e.g., for a sample comprising one or more cells); delivery reagents (e.g., for delivering the one or more engineered reporter polynucleotides, or delivery vehicles of the same, into the one or more cells of the sample); expression reagents (e.g., for inducing expression of the engineered reporter polynucleotides in the cells), and/or detection reagents (e.g., for detecting a signal generated by the expression of the engineered reporter polynucleotides in the cells). In an embodiment, they are included in one or more compartments and/or locations within the device in a free-dried, lyophilized or some other form. Devices can contain or be configured for optical-based readouts, lateral flow readouts, electrical readouts or others that are described herein and will be appreciated in view of the description provided herein.

### Discrete Volumes

In an embodiment the devices can include individual discrete volumes. In certain embodiments, the engineered reporter polynucleotide detection system is comprised in or bound to each discrete volume in the device. Each discrete volume may comprise a different engineered reporter polynucleotide specific for a different cell type, and/or cell state (e.g., a diseased or abnormal cell type and/or cell state). In certain embodiments, a sample is exposed to a solid substrate comprising more than one discrete volume each comprising an engineered reporter polynucleotide specific for a different cell type, and/or cell state. Not being bound by a theory, each engineered reporter polynucleotide will interact with a specific cell type, and/or cell state from the sample and the sample does not need to be divided into separate assays. Thus, a valuable sample may be preserved.

Several substrates and configurations of devices capable of defining multiple individual discrete volumes within the device may be used. As used herein “individual discrete volume” refers to a discrete space, such as a container, receptacle, or other arbitrary defined volume or space that can be defined by properties that prevent and/or inhibit migration of samples and/or reagents, for example a volume or space defined by physical properties such as walls, for example the walls of a well, tube, or a surface of a droplet, which may be impermeable or semipermeable, or as defined by other means such as chemical, diffusion rate limited, electro-magnetic, or light illumination, or any combination thereof that can contain a target molecule and a indexable nucleic acid identifier (for example nucleic acid barcode). By “diffusion rate limited” (for example diffusion defined volumes) is meant spaces that are only accessible to certain molecules or reactions because diffusion constraints effectively defining a space or volume as would be the case for two parallel laminar streams where diffusion will limit the migration of samples and/or reagents from one stream to the other. By “chemical” defined volume or space is meant spaces where only certain target molecules can exist because of their chemical or molecular properties, such as size, where for example gel beads may exclude certain species from entering the beads but not others, such as by surface charge, matrix size or other physical property of the bead that can allow selection of species that may enter the interior of the bead. By “electro-magnetically” defined volume or space is meant spaces where the electro-magnetic properties of the target molecules or their supports such as charge or magnetic properties can be used to define certain regions in a space such as capturing magnetic particles within a magnetic field or directly on magnets. By “optically” defined volume is meant any region of space that may be defined by illuminating it with visible, ultraviolet, infrared, or other wavelengths of light such that only target molecules within the defined space or volume may be labeled. One advantage to the use of non-walled, or semipermeable discrete volumes is that some reagents, such as buffers, chemical activators, or other agents may be passed through the discrete volume, while other materials, such as target molecules, may be maintained in the discrete volume or space. Typically, a discrete volume will include a fluid medium, (for example, an aqueous solution, an oil, a buffer, and/or a media capable of supporting cell growth) suitable for: delivery (e.g., to a cell or a population of cells) of the one or more engineered reporter polynucleotides, or delivery vehicles comprising the same; expression of the same in the cell or the population of cells; and/or providing the detectable signal, under conditions that permit the delivery, expression, and/or detection. Exemplary discrete volumes or spaces useful in the disclosed methods include droplets (for example, microfluidic droplets and/or emulsion droplets), hydrogel beads or other polymer structures (for example poly-ethylene glycol di-acrylate beads or agarose beads), tissue slides (for example, fixed formalin paraffin embedded tissue slides with particular regions, volumes, or spaces defined by chemical, optical, or physical means), microscope slides with regions defined by depositing reagents in ordered arrays or random patterns, tubes (such as, centrifuge tubes, microcentrifuge tubes, test tubes, cuvettes, conical tubes, and the like), bottles (such as glass bottles, plastic bottles, ceramic bottles, Erlenmeyer flasks, scintillation vials and the like), wells (such as wells in a plate), plates, pipettes, or pipette tips among others. In certain embodiments, the compartment is an aqueous droplet in a water-in-oil emulsion. In specific embodiments, any of the applications, methods, or systems described herein requiring exact or uniform volumes may employ the use of an acoustic liquid dispenser.

### Samples

The device can be configured to hold, store, collect, receive, process and/or otherwise manipulate a sample and/or detect a component thereof. In an embodiment, the sample is a solid, semisolid, or liquid. In an embodiment, the sample is a biological sample. In an embodiment, the sample is obtained from a subject. In an embodiment, the sample is a bodily fluid. In an embodiment, the bodily fluid is saliva or nasal secretions. In an embodiment, the sample is not a bodily fluid but contains one or more cells from the subject, such as hair cells, skin cells, solid tissue or portion thereof, or tumor cells. In an embodiment, the sample is obtained from a plant. In an embodiment, the sample is an environmental sample, such as air, soil, water, or a sample of molecules, organisms, viruses, and other particles present on an object surface. In an embodiment, the sample is a feedstuff or foodstuff or component thereof. Other exemplary samples that may be analyzed using the systems and devices described herein include biological samples of a subject or environmental samples. Environmental samples may include surfaces or fluids. The biological samples may include, but are not limited to, saliva, blood, plasma, sera, stool, urine, sputum, mucous, lymph, synovial fluid, spinal fluid, cerebrospinal fluid, sweat, milk, semen, a swab from skin or a mucosal membrane, or combination thereof. In an example embodiment, the environmental sample is taken from a solid surface, such as a surface used in the preparation of food or other sensitive compositions and materials.

A sample for use with the invention may be a biological or environmental sample, such as a surface sample, a fluid sample, or a food sample (fresh fruits or vegetables, meats). Food samples may include a beverage sample, a paper surface, a fabric surface, a metal surface, a wood surface, a plastic surface, a soil sample, a freshwater sample, a wastewater sample, a saline water sample, exposure to atmospheric air or other gas sample, or a combination thereof. For example, household/commercial/industrial surfaces made of any materials including, but not limited to, metal, wood, plastic, rubber, or the like, may be swabbed and tested for contaminants. Soil samples may be tested for the presence of pathogenic bacteria or parasites, or other microbes, both for environmental purposes and/or for human, animal, or plant disease testing. Water samples such as freshwater samples, wastewater samples, or saline water samples can be evaluated for cleanliness and safety, and/or potability, to detect the presence of, for example, Cryptosporidium parvum, Giardia lamblia, or other microbial contamination. In further embodiments, a biological sample may be obtained from a source including, but not limited to, a tissue sample, saliva, blood, plasma, sera, stool, urine, sputum, mucous, lymph, synovial fluid, spinal fluid, cerebrospinal fluid, ascites, pleural effusion, seroma, pus, bile, aqueous or vitreous humor, transudate, exudate, sweat, milk, semen, or swab of skin or a mucosal membrane surface. In some particular embodiments, an environmental sample or biological samples may be crude samples and/or the one or more target molecules may not be purified or amplified from the sample prior to application of the method. Identification of microbes may be useful and/or needed for any number of applications, and thus any type of sample from any source deemed appropriate by one of skill in the art may be used in accordance with the invention.

In particular embodiments, the methods and systems can be utilized for direct detection from patient samples. In an aspect, the methods and systems can further allow for direct detection from patient samples with a visual readout to further facilitate field-deployability. In an aspect, a field depoloyable version can include, for example the lateral flow devices and systems as described herein, and/or colorimetric detection. The methods and systems can be utilized to detect specific cell types and/or cell states of one or more cells in a sample. In an aspect, the sample is from a nasophyringeal swab or a saliva sample.

### Flexible Substrates

In certain example embodiments, the device comprises a flexible material substrate on which a number of spots or discrete volumes may be defined. Flexible substrate materials suitable for use in diagnostics and biosensing are known within the art. The flexible substrate materials may be made of plant derived fibers, such as cellulosic fibers, or may be made from flexible polymers such as flexible polyester films and other polymer types. Within each defined spot, reagents of the system described herein are applied to the individual spots. Each spot may contain the same reagents except for a different engineered reporter polynucleotide or set of engineered reporter polynucleotides to screen for multiple cell types, and/or cell states in a sample at once. Thus, the systems and devices herein may be able to screen samples from multiple sources (e.g. multiple clinical samples from different individuals) for the presence of the same cell types, and/or cell states, or a limited number of cell types, and/or cell states, or aliquots of a single sample (or multiple samples from the same source) for the presence of multiple different cell types, and/or cell states in the sample. In certain example embodiments, the elements of the systems described herein are freeze dried onto the paper or cloth substrate. Example flexible material-based substrates that may be used in certain example devices are disclosed in Pardee et al. Cell. 2016, 165 (5): 1255-66 and Pardee et al. Cell. 2014, 159 (4): 950-54. Suitable flexible material-based substrates for use with biological fluids, including blood are disclosed in International Patent Application Publication No. WO/2013/071301 entitled “Paper based diagnostic test” to Shevkoplyas et al. U.S. Patent Application Publication No. 2011/0111517 entitled “Paper-based microfluidic systems” to Siegel et al. and Shafiee et al. “Paper and Flexible Substrates as Materials for Biosensing Platforms to Detect Multiple Biotargets” Scientific Reports 5:8719 (2015). Further flexible based materials, including those suitable for use in wearable diagnostic devices are disclosed in Wang et al. “Flexible Substrate-Based Devices for Point-of-Care Diagnostics” Cell 34 (11): 909-21 (2016). Further flexible based materials may include nitrocellulose, polycarbonate, methylethyl cellulose, polyvinylidene fluoride (PVDF), polystyrene, or glass (see e.g., US20120238008). In certain embodiments, discrete volumes are separated by a hydrophobic surface, such as but not limited to wax, photoresist, or solid ink.

In an embodiment, the substrate, such as a flexible substrate, is a single use substrate, such as swab, strip, or cloth that is used to swab a surface or sample fluid or is placed in a prepared sample for detection by an assay described herein. Similarly, the single use substrate may be used to swab other surfaces for detection of certain cell type and/or cell state in one or more cells, such as for use in security screening. Single use substrates may also have applications in forensics, where the engineered reporter polynucleotide detection systems are designed to detect, for example specific cell types and/or cell states in one or more cells that may be used to identify a suspect, or to determine the type of biological matter present in a sample. Likewise, the single use substrate could be used to collect a sample from a patient-such as a saliva sample from the mouth- or a swab of the skin.

### Microfluidic Devices

In certain example embodiments, the device is configured as a microfluidic device. It will be appreciated that the microfluidic device can incorporate a chip, cartridge, flexible substrate, lateral flow strip, and/or other components described elsewhere herein. In an embodiment the microfluidic device can be configured to drive a sample through the device such that it contacts one or more engineered reporter polynucleotide detection system reagents (such as those that may be present on a flexible substrate within the device) and thus carries out an engineered reporter polynucleotide detection reaction. In an embodiment, the microfluidic device is configured to generate and/or merge different droplets (i.e., individual discrete volumes). For example, a first set of droplets may be formed containing samples to be screened and a second set of droplets formed containing the elements of the engineered reporter polynucleotide detection systems described herein. The first and second set of droplets are then merged and then diagnostic methods as described herein are carried out on the merged droplet set. Microfluidic devices disclosed herein may be silicone-based chips and may be fabricated using a variety of techniques, including, but not limited to, hot embossing, molding of elastomers, injection molding, LIGA, soft lithography, silicon fabrication and related thin film processing techniques. Suitable materials for fabricating the microfluidic devices include, but are not limited to, cyclic olefin copolymer (COC), polycarbonate, poly(dimethylsiloxane) (PDMS), and poly(methylacrylate) (PMMA). In one embodiment, soft lithography in PDMS may be used to prepare the microfluidic devices. For example, a mold may be made using photolithography which defines the location of flow channels, valves, and filters within a substrate. The substrate material is poured into a mold and allowed to set to create a stamp. The stamp is then sealed to a solid support, such as but not limited to, glass. Due to the hydrophobic nature of some polymers, such as PDMS, which absorbs some proteins and may inhibit certain biological processes, a passivating agent may be necessary (Schoffner et al. Nucleic Acids Research, 1996, 24:375-379). Suitable passivating agents are known in the art and include, but are not limited to, silanes, parylene, n-Dodecyl-b-D-matoside (DDM), pluronic, Tween-20, other similar surfactants, polyethylene glycol (PEG), albumin, collagen, and other similar proteins and peptides.

In certain example embodiments, the system and/or device may be adapted for conversion to a flow-cytometry readout in or allow to sensitive and quantitative measurements of millions of cells in a single experiment and improve upon existing flow-based methods, such as the PrimeFlow assay. In certain example embodiments, cells may be cast in droplets containing unpolymerized gel monomer, which can then be cast into single-cell droplets suitable for analysis by flow cytometry. One or more components of the engineered reporter polynucleotide detection system may be cast into the droplet comprising unpolymerized gel monomer. Upon polymerization of the gel monomer, a bead forms within a droplet. Because gel polymerization is through free-radical formation, the system components become covalently bound to the gel.

An example of microfluidic device that may be used in the context of the invention is described in Hou et al. “Direct Detection and drug-resistance profiling of bacteremias using inertial microfluidics” Lap Chip. 15 (10): 2297-2307 (2016). Further LOC embodiments are described elsewhere herein.

### Lateral Flow Devices

In certain embodiments, the detection assay can be provided on a lateral flow device, as described in International Publication WO 2019/071051, incorporated herein by reference. The lateral flow device can be adapted to detect one or more specific cell types and/or cell states in one or more cells. The lateral flow device may comprise a flexible substrate, such as a paper substrate or a flexible polymer-based substrate, which can include freeze-dried reagents for detection assays with a visual readout of the assay results. See, WO 2019/071051 at [0145]-[0151] and Example 2, specifically incorporated herein by reference. In an aspect, lyophilized reagents can include preferred excipients that aid in rate of reaction, specificity, or other variables. The excipients may comprise trehalose, histidine, and/or glycine. In certain embodiments, the coronavirus assay can be utilized with isothermal amplification reagents, allowing amplification without complex instrumentation that may be unavailable in the field, as described in WO 2019/071051. Accordingly, the assay can be adapted for field diagnostics, including use of visual readout on a lateral flow device, rapid, sensitive detection and can be deployed for early and direct detection. Colorimetric detection can be utilized and may be particularly suited for field deployable applications, as described in International Application PCT/US2019/015726, published as WO2019/148206. In particular, colorimetric detection can be as described in WO2019/148206 at FIGS. 102, 105, 107-111 and [00306]-[00324], incorporated herein by reference.

In one embodiment, the invention provides a lateral flow device comprising a substrate comprising a first end and a second end. The first end may comprise a sample loading portion, a first region comprising a detectable ligand, two or more CRISPR effector systems, two or more detection constructs, and one or more first capture regions, each comprising a first binding agent. The substrate may also comprise two or more second capture regions between the first region of the first end and the second end, each second capture region comprising a different binding agent. Each of the two or more CRISPR effector systems may comprise a CRISPR effector protein and one or more guide sequences, each guide sequence configured to bind one or more expression products of the engineered reporter polynucleotide.

The embodiments disclosed herein are directed to lateral flow detection devices that comprise an engineered reporter polynucleotide detection system described herein. The device may comprise a lateral flow substrate for detecting an engineered reporter polynucleotide detection system reaction. Substrates suitable for use in lateral flow assays are known in the art. These may include but are not necessarily limited to membranes or pads made of cellulose and/or glass fiber, polyesters, nitrocellulose, or absorbent pads (J Saudi Chem Soc 19 (6): 689-705; 2015), and other embodiments further described herein. One or more components of the engineered reporter polynucleotide detection system, i.e., the one or more engineered reporter polynucleotides and corresponding detection reagents, are added to the lateral flow substrate at a defined reagent portion of the lateral flow substrate, typically on one end of the lateral flow substrate. The lateral flow substrate further comprises a sample portion. The sample portion may be equivalent to, continuous with, or adjacent to the reagent portion.

### Lateral Flow Substrate

In an embodiment, the device is a lateral flow device. In an embodiment, the lateral flow device can be composed of an engineered reporter polynucleotide detection system described elsewhere herein and a lateral flow substrate for carrying out the detection reaction in the sample. In certain example embodiments, a lateral flow device comprises a lateral flow substrate on which detection can be performed. Substrates suitable for use in lateral flow assays are known in the art. These may include, but are not necessarily limited to, membranes or pads made of cellulose and/or glass fiber, polyesters, nitrocellulose, or absorbent pads (J Saudi Chem Soc 19 (6): 689-705; 2015).

Lateral support substrates comprise a first and second end, and one or more capture regions that each comprise binding agents. The first end may comprise a sample loading portion, a first region comprising a detectable ligand, two or more CRISPR effector systems, two or more detection constructs, and one or more first capture regions, each comprising a first binding agent. The substrate may also comprise two or more second capture regions between the first region of the first end and the second end, each second capture region comprising a different binding agent. Each of the two or more CRISPR effector systems may comprise a CRISPR effector protein and one or more guide sequences, each guide sequence configured to bind one or more expression products of the engineered reporter polynucleotide. The lateral flow substrates may be configured to detect a CRISPR-Cas collateral activity detection reaction.

Lateral support substrates may be located within a housing (see for example, “Rapid Lateral Flow Test Strips” Merck Millipore 2013). The housing may comprise at least one opening for loading samples and a second single opening or separate openings that allow for reading of detectable signal generated at the first and second capture regions.

The embodiments disclosed herein can be prepared in freeze-dried format for convenient distribution and point-of-care (POC) applications. Such embodiments are useful in multiple scenarios in human health including, for example, disease detection. Accordingly, the lateral substrate comprising one or more of the elements of the system, including engineered reporter polynucleotide, delivery systems of the same, expression reagents, and/or detection reagents may be freeze-dried to the lateral flow substrate and packaged as a ready to use device. Alternatively, all or a portion of the elements of the system may be added to the reagent portion of the lateral flow substrate at the time of using the device.

### First End and Second End of the Substrate

The substrate of the lateral flow device comprises a first and second end. The engineered reporter polynucleotide detection system described herein, i.e., one or more engineered reporter polynucleotides and one or more corresponding detection reagents, is added to the lateral flow substrate at a defined reagent portion of the lateral flow substrate, typically on a first end of the lateral flow substrate. The lateral flow substrate further comprises a sample portion. The sample portion may be equivalent to, continuous with, or adjacent to the reagent portion.

In certain example embodiments, the first end comprises a first region. The first region comprises a detectable ligand, two or more CRISPR effector systems, two or more detection constructs, and one or more first capture regions, each comprising a first binding agent.

### Capture Regions

The lateral flow substrate can comprise one or more capture regions. In embodiments the first end of the lateral flow substrate comprises one or more first capture regions, with two or more second capture regions between the first region of the first end of the substrate and the second end of the substrate. The capture regions may be provided as a capture line, typically a horizontal line running across the device, but other configurations are possible. The first capture region is proximate to and on the same end of the lateral flow substrate as the sample loading portion.

### Binding Agents

Specific binding-integrating molecules comprise any members of binding pairs that can be used in the present invention. Such binding pairs are known to those skilled in the art and include, but are not limited to, antibody-antigen pairs, enzyme-substrate pairs, receptor-ligand pairs, and streptavidin-biotin. In addition to such known binding pairs, novel binding pairs may be specifically designed. A characteristic of binding pairs is the binding between the two members of the binding pair.

A first binding agent that specifically binds a target molecule, such as a barcode or other sequence in the reporter polynucleotide, is fixed or otherwise immobilized to the first capture region. The second capture region is located towards the opposite end of the lateral flow substrate from the first capture region. A second binding agent is fixed or otherwise immobilized at the second capture region. The second binding agent specifically binds the first binding agent and/or target molecule, or the second binding agent may bind a detectable ligand. For example, the detectable ligand may be a particle, such as a colloidal particle, that when it aggregates can be detected visually, and generates a detectable positive signal. The particle may be modified with an antibody that specifically binds the second molecule on the reporter construct. If the reporter construct is not cleaved it will facilitate accumulation of the detectable ligand at the first binding region. If the reporter construct is cleaved the detectable ligand is released to flow to the second binding region. In such an embodiment, the second binding region comprises a second binding agent capable of specifically or non-specifically binding the detectable ligand on the antibody of the detectable ligand. Binding agents can be, for example, antibodies, that recognize a particular affinity tag. Such binding agents can further contain, for example, detectable labels, such as isotope labels and/or nucleic acid barcodes. A barcode is a short sequence of nucleotides (for example, DNA, RNA, or combinations thereof) that is used as an identifier. A nucleic acid barcode may have a length of 4-100 nucleotides and be either single or double-stranded. Methods for identifying cells with barcodes are known in the art. Accordingly, guide RNAs of the CRISPR effector systems described herein may be used to detect the barcode.

### Detectable Ligands

The first region is loaded with a detectable ligand, such as those disclosed herein, for example a gold nanoparticle. The detectable ligand may be a particle, such as a colloidal particle, that when it aggregates can be detected visually. The particle may be modified with an antibody that specifically binds the second molecule on the reporter construct. If the reporter construct is not cleaved it will facilitate accumulation of the detectable ligand at the first binding region. If the reporter construct is cleaved the detectable ligand is released to flow to the second binding region. In such an embodiment, the second binding agent is an agent capable of specifically or non-specifically binding the detectable ligand on the antibody on the detectable ligand. Examples of suitable binding agents for such an embodiment include, but are not limited to, protein A and protein G. In some examples, the detectable ligand is a gold nanoparticle, which may be modified with a first antibody, such as an anti-FITC antibody.

### Lateral Flow Detection Constructs

The first region also comprises a detection construct. In one example embodiment, a RNA detection construct and a CRISPR effector system (a CRISPR effector protein and one or more guide sequences configured to bind to one or more target sequences) as disclosed herein. In one example embodiment, and for purposes of further illustration, the RNA construct may comprise a FAM molecule on a first end of the detection construction and a biotin on a second end of the detection construct. Upstream of the flow of solution from the first end of the lateral flow substrate is a first test band. The test band may comprise a biotin ligand. Accordingly, when the RNA detection construct is present it its initial state, i.e., in the absence of target, the FAM molecule on the first end will bind the anti-FITC antibody on the gold nanoparticle, and the biotin on the second end of the RNA construct will bind the biotin ligand allowing for the detectable ligand to accumulate at the first test, generating a detectable signal. Generation of a detectable signal at the first band indicates the absence of the target ligand. In the presence of target, the CRISPR effector complex forms and the CRISPR effector protein is activated resulting in cleavage of the RND detection construct. In the absence of intact RNA detection construct the colloidal gold will flow past the second strip. The lateral flow device may comprise a second band, upstream of the first band. The second band may comprise a molecule capable of binding the antibody-labeled colloidal gold molecule, for example an anti-rabbit antibody capable of binding a rabbit anti-FITC antibody on the colloidal gold. Therefore, in the presence of one or more targets, the detectable ligand will accumulate at the second band, indicating the presence of the one or more targets in the sample.

In an embodiment, the first end of the lateral flow device comprises two detection constructs and each of the two detection constructs comprises an RNA or DNA oligonucleotide, comprising a first molecule on a first end and a second molecule on a second end. The first molecule and the second molecule may be linked by an RNA or DNA linker.

In an embodiment, the first molecule on the first end of the first detection construct may be FAM and the second molecule on the second end of the first detection construct may be biotin, or vice versa. In an embodiment, the first molecule on the first end of the second detection construct may be FAM and the second molecule on the second end of the second detection construct may be Digoxigenin (DIG), or vice versa.

In an embodiment, the first end may comprise three detection constructs, wherein each of the three detection constructs comprises an RNA or DNA oligonucleotide, comprising a first molecule on a first end and a second molecule on a second end. In specific embodiments, the first and second molecules on the detection constructs comprise Tye 665 and Alexa 488; Tye 665 and FAM, and Tye 665 and Digoxigenin (DIG), respectively.

In an embodiment, the first end of the lateral flow device comprises two or more CRISPR effector systems, also referred to as a CRISPR-Cas or CRISPR system. In an embodiment, such a CRISPR effector system may include a CRISPR effector protein and one or more guide sequences configured to bind to one or more target sequences.

### Samples

When utilizing the detection systems with a lateral flow substrate, samples to be screened are loaded at the sample loading portion of the lateral flow substrate. The samples must be liquid samples or samples dissolved in an appropriate solvent, usually aqueous. The liquid sample reconstitutes the engineered reporter polynucleotide detection reagents such that an engineered reporter polynucleotide detection reaction can occur. The liquid sample begins to flow from the sample portion of the substrate towards the first and second capture regions. Exemplary samples are described in greater detail elsewhere herein. See also WO 2019/071051, which is incorporated by reference herein.

### Cartridges and Chips

The cartridge, also referred to herein as a chip, according to the present invention comprises a series of components of ampoules and chambers that are communicatively coupled with one or more other components on the cartridge. The coupling is typically a fluidic communication, for example, via channels. The cartridge may comprise a membrane that seals one or more of the chambers and/or ampoules. In an aspect, the membrane allows for storage of reagents, buffers and other solid or fluid components which cover and seal the cartridge. The membrane can be configured to be punctured, pierced or otherwise released from sealing or covering one or more components of the cartridge by a means for releasing reagents. In an embodiment, the cartridge contains one or more wells, substrates (e.g., a flexible substrate), or other discrete volumes.

In an embodiment, the device is configured as lab-on-chip (LOC) diagnostic system. In an embodiment, the LOC is configured as a wireless lab-on-chip (LOC) diagnostic sensor system (see e.g., U.S. Pat. No. 9,470,699). In certain embodiments, CRISPR-Cas collateral activity detection assay is performed in a LOC controlled and/or read by a wireless device (e.g., a cell phone, a personal digital assistant (PDA), a tablet) and results and/or reaction are reported to and/or measured by said device. In an embodiment, the LOC may be a microfluidic device. The LOC may be a passive chip, wherein the chip is powered and controlled through a wireless device. In certain embodiments, the LOC includes a microfluidic channel for holding reagents and a channel for introducing a sample. In certain embodiments, a signal from the wireless device delivers power to the LOC and activates mixing of the sample and assay reagents.

Specifically, in the case of the present invention, the system may include an engineered reporter polynucleotide specific for a cell type and/or cells state. Upon activation of the LOC, the microfluidic device may mix the sample and assay reagents. Upon mixing, a sensor detects a signal and transmits the results to the wireless device. In certain embodiments, the unmasking agent is a conductive RNA molecule. The conductive RNA molecule may be attached to the conductive material. Conductive molecules can be conductive nanoparticles, conductive proteins, metal particles that are attached to the protein or latex or other beads that are conductive. In certain embodiments, if DNA or RNA is used then the conductive molecules can be attached directly to the matching DNA or RNA strands. The release of the conductive molecules may be detected across a sensor. The assay may be a one step process. Lab-on-the chip technology is well described in the scientific literature and consists of multiple microfluidic channels, input or chemical wells. Reactions in wells can be measured using radio frequency identification (RFID) tag technology since conductive leads from RFID electronic chip can be linked directly to each of the test wells. An antenna can be printed or mounted in another layer of the electronic chip or directly on the back of the device. Furthermore, the leads, the antenna and the electronic chip can be embedded into the LOC chip, thereby preventing shorting of the electrodes or electronics. Since LOC allows complex sample separation and analyses, this technology allows LOC tests to be done independently of a complex or expensive reader. Rather a simple wireless device such as a cell phone or a PDA can be used. In one embodiment, the wireless device also controls the separation and control of the microfluidics channels for more complex LOC analyses. In one embodiment, a LED and other electronic measuring or sensing devices are included in the LOC-RFID chip. Not being bound by a theory, this technology is disposable and allows complex tests that require separation and mixing to be performed outside of a laboratory.

As noted above, certain embodiments enable the use of an expression product binding beads to concentrate a target expression product but that do not require elution of the isolated expression product. Thus, in certain example embodiments, the cartridge may further comprise an activatable magnet, such as an electro-magnet. A means for activating the magnet may be located on the device, or the means for supplying the magnet or activating the magnet on the cartridge may be provided by a second device, such as those disclosed in further detail below.

The overall size of the device may be between 10, 15, 20, 25, 30, 35, 40, 45, 55, 60, 65, 70, 75, 80, 85, 90, 95, or 100 mm in width, and 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, or 200 mm. The sizing of ampoules, chambers, and channels can be selected to be in line with the reaction volumes discussed herein and to fit within the general size parameters of the overall cartridge.

### Ampoules

The ampoules, also referred to as blisters, allow for storage and release of reagents throughout the cartridge. Ampoules can include liquid or solid reagents, for example, expression reagents in one ampoule and detection reagents in another ampoule. The reagents can be as described elsewhere herein and can be adapted for the use in the cartridge. The ampoule may be sealed by a film that allows for the bursting, puncture or other release of the contents of the ampoules. See, e.g., Becker, H. & Gärtner, C. Microfluidics-enabled diagnostic systems: markets, challenges, and examples. In Microchip Diagnostics: Methods and Protocols (eds Taly, V. et al.) (Springer, New York, 2017); Czurratis et al., doi: 10.1088/0960-1317/25/4/045002. Considerations for ampoules can include as discussed in, for example, Smith, S., et al., Blister pouches for effective reagent storage on microfluidic chips for blood cell counting. Microfluid Nanofluid 20, 163 (2016). DOI: 10.1007/s10404-016-1830-2. In an aspect, the seal is a frangible seal formed of a composite-layer film that is assembled to the cartridge main body or other part of the device. While referred to herein as an ampoule, the ampoule may comprise a cavity on a chip which comprises a sealed film that is opened by the release means.

### Chambers

The chambers on the chip may located and sized for fluidic communication via channels or other communication means with ampoules and/or other chambers on the chip. A chamber for receiving a sample can be provided. The sample can be injected, placed in a receptacle into the chamber for receiving a sample, or otherwise transferred to the chamber. An expression chamber may comprise, for example, capture beads, that may be used for concentration and/or extraction of the desired expression products from the sample. Alternatively, the beads may be comprised in an ampoule comprising lysis reagents that are in fluidic communication with the lysis chamber. An amplification chamber may also be provided with, for example, one or more lyophilized components of the system in the amplification chamber and/or communicatively connected to an ampoule comprising one or more components of the amplification reaction.

When the cartridge comprises a magnet, it may be configured near one or more of the chambers. In an aspect, the magnet is near the expression well, and may be configured such that the device has a means for activating the magnet. Embodiments comprising a magnet in the cartridge may be utilized with methodologies using magnetic beads for extraction of particular target expression products.

### System for Detection Assays

A system configured for use with the cartridge and to perform an assay, also referred to as a sample analysis apparatus, detection system or detection device, is configured system to receive the cartridge and conduct an assay comprising expression of the engineered reporter polynucleotide and detection of target expression products on the cartridge. The system may comprise: a body; a door housing which may be provided in an opened state or a closed state and configured to be coupled to the body of the sample analysis apparatus by a hinge or other closure means; a cartridge accommodating unit included in the detection system and configured to accommodate the cartridge. The system may further comprise one or more means for releasing reagents for expression and/or detection; one or more heating means for expression and/or detection, a means for mixing reagents for expression and/or detection, and/or a means for reading the results of the assay. The device may further comprise a user interface for programming the device and/or readout of the results of the assay.

### Means for Release of Reagents

The system may comprise means for releasing reagents for extraction, amplification and/or detection. Release of reagents can be performed by a crushing, puncturing, applying heat or pressure until burst, cutting, or other means for the opening of the ampoule and release of contents. e.g., Becker, H. & Gärtner, C. Microfluidics-enabled diagnostic systems: markets, challenges, and examples. In Microchip Diagnostics: Methods and Protocols (eds Taly, V. et al.) (Springer, New York, 2017); Czurratis et al., doi: 10.1088/0960-1317/25/4/045002. Mechanical actuators.

### Heating Means

The heating means or heating element can be provided, for example, by electrical or chemical elements. One or more heating means can be utilized, or circuits providing regulation of temperature to one or more locations within the detection device can be utilized. In an embodiment, the device is configured to comprise a heating means for heating the expression and/or detection chambers of the cartridge, sample vessel or other part of the device. In an aspect, the heating element is disposed under the expression and/or detection well. The system can be designed with one or more heating means for expression and/or detection. In an embodiment, the device does not include a power source. In an embodiment, the heating element provides heat of about 65, 60, 55, 50, 45, 40, 39, 38, 37, 36, 35, 34, 33, 32, 31, 30, 29, 28, 27, 26, 25 degrees C. or less. In an embodiment, the device does not contain any heating element.

### Power Sources

In an embodiment, the device can include a power source. The power source can be coupled to one or more of the components of the device. In an embodiment, the power source is electrically coupled to one or more components of the device so as to provide electrical energy to the cone or more components. Suitable power sources that can be incorporated with the device are batteries (single use and rechargeable), solar powered power sources and batteries. In an embodiment, the power source can be coupled to an outside power source (e.g., an electric power grid) so as to recharge the on-board power source. In an embodiment, the device does not include a power source.

### Mixing Means

A means for mixing reagents for expression and/or detection can be provided. A means for mixing reagents may comprise a means for mixing one or more fluids, or a fluid with a solid or lyophilized reaction mixture can also be provided. Means for mixing that disturb the laminar flow can be provided. In an aspect, the mixing means is a passive mixer, in another aspect, the mixing means is an active mixer. See, e.g. Nam-Trung Nguyen and Zhigang Wu 2005 J. Micromech. Microeng. 15 R1, doi: 10.1088/0960-1317/15/2/R01 for discussion of mixing approaches. In an aspect, the active mixer can be based on external sources such as pressure, temperature, hydrodynamics (with electrical or magnetic forces), dielectrophoresis, electrokinetics, or acoustics. Examples of passive mixing means can be provided by use of geometric approaches, such as a curved path or channel, see, e.g., U.S. Pat. No. 7,160,025, or an expansion/contraction of a channel cross section or diameter. When the cartridge is utilized with beads, channels and wells are configured and sized for the flow of beads.

### Means for Reading the Results of the Assay

A means for reading the results of the assay can be provided in the system. The means for reading the results of the assay will depend in part on the type of detectable signal generated by the assay. In particular embodiments, the assay generates a detectable fluorescent or color readout. In these instances, the means for reading the results of the assay will be an optic means, for example a single channel or multi-channel optical means such as a fluorimeter, colorimeter or other spectroscopic sensor.

A combination of means for reading the results of the assay can be utilized, and may include readings such as turbidity, temperature, magnetic, radio, or electrical properties and or optical properties, including scattering, polarization effects, etc.

The system may further comprise a user interface for programming the device and/or readout of the results of the assay. The user interface may comprise an LED screen. The system can be further configured for a USB port that can allow for docking of four or more devices.

In an aspect, the system comprises a means for activating a magnet that is disposed within or on the cartridge.

### Wearable Devices

The systems described herein, may further be incorporated into wearable medical devices that assess biological samples, such as biological fluids or an environmental sample, of a subject or in a subject's environment outside the clinic setting and report the outcome of the assay remotely to a central server accessible by a medical care professional. In an embodiment the device may include the ability to self-sample blood, saliva, sweat, such as the devices disclosed in U.S. Patent Application Publication No. 2015/0342509 entitled “Needle-free Blood Draw to Peeters et al., U.S. Patent Application Publication No. 2015/0065821 entitled “Nanoparticle Phoresies” to Andrew Conrad.

In an embodiment, the device is configured as a dosimeter or badge that serves as a sensor or indicator such that the wearer is notified of exposure to certain microbes or other agents. For example, the systems described herein may be used to detect a particular pathogen. Likewise, aptamer-based embodiments disclosed above may be used to detect both polypeptide as well as other agents, such as chemical agents, to which a specific aptamer may bind. Such a device may be useful for surveillance of soldiers or other military personnel, as well as clinicians, researchers, hospital staff, and the like, in order to provide information relating to exposure to potentially dangerous microbes as quickly as possible, for example for biological or chemical warfare agent detection. In other embodiments, such a surveillance badge may be used for preventing exposure to dangerous microbes or pathogens in immunocompromised patients, burn patients, patients undergoing chemotherapy, children, or elderly individuals.

### Other Device Features

In certain example embodiments, the device may comprise individual wells, such as microplate wells. The size of the microplate wells may be the size of standard 6, 24, 96, 384, 1536, 3456, or 9600 sized wells. In certain example embodiments, the elements of the systems described herein may be freeze dried and applied to the surface of the well prior to distribution and use.

The devices disclosed herein may further comprise inlet and outlet ports, or openings, which in turn may be connected to valves, tubes, channels, chambers, and syringes and/or pumps for the introduction and extraction of fluids into and from the device. The devices may be connected to fluid flow actuators that allow directional movement of fluids within the microfluidic device. Example actuators include, but are not limited to, syringe pumps, mechanically actuated recirculating pumps, electroosmotic pumps, bulbs, bellows, diaphragms, or bubbles intended to force movement of fluids. In certain example embodiments, the devices are connected to controllers with programmable valves that work together to move fluids through the device. In certain example embodiments, the devices are connected to the controllers discussed in further detail below. The devices may be connected to flow actuators, controllers, and sample loading devices by tubing that terminates in metal pins for insertion into inlet ports on the device.

As shown herein the elements of the system are stable when freeze dried or lyophilized, therefore embodiments that do not require a supporting device are also contemplated, i.e., the system may be applied to any surface or fluid that will support the reactions disclosed herein and allow for detection of a positive detectable signal from that surface or solution. In addition to freeze-drying, the systems may also be stably stored and utilized in a pelletized form. Polymers useful in forming suitable pelletized forms are known in the art.

The devices disclosed herein may also include elements of point of care (POC) devices known in the art for analyzing samples by other methods. See, for example St John and Price, “Existing and Emerging Technologies for Point-of-Care Testing” (Clin Biochem Rev. 2014 August; 35 (3): 155-167).

Radio frequency identification (RFID) tag systems include an RFID tag that transmits data for reception by an RFID reader (also referred to as an interrogator). In a typical RFID system, individual objects (e.g., store merchandise) are equipped with a relatively small tag that contains a transponder. The transponder has a memory chip that is given a unique electronic product code. The RFID reader emits a signal activating the transponder within the tag through the use of a communication protocol. Accordingly, the RFID reader is capable of reading and writing data to the tag. Additionally, the RFID tag reader processes the data according to the RFID tag system application. Currently, there are passive and active type RFID tags. The passive type RFID tag does not contain an internal power source, but is powered by radio frequency signals received from the RFID reader. Alternatively, the active type RFID tag contains an internal power source that enables the active type RFID tag to possess greater transmission ranges and memory capacity. The use of a passive versus an active tag is dependent upon the particular application.

Since the electrical conductivity of the surface area can be measured precisely quantitative results are possible on the disposable wireless RFID electro-assays. Furthermore, the test area can be very small allowing for more tests to be done in a given area and therefore resulting in cost savings. In certain embodiments, separate sensors each associated with a different CRISPR effector protein and guide RNA immobilized to a sensor are used to detect multiple target molecules. Not being bound by a theory, activation of different sensors may be distinguished by the wireless device.

In addition to the conductive methods described herein, other methods may be used that rely on RFID or Bluetooth as the basic low-cost communication and power platform for a disposable RFID assay. For example, optical means may be used to assess the presence and level of a given target molecule. In certain embodiments, an optical sensor detects unmasking of a fluorescent masking agent.

In certain embodiments, the device of the present invention may include handheld portable devices for diagnostic reading of an assay (see e.g., Vashist et al., Commercial Smartphone-Based Devices and Smart Applications for Personalized Healthcare Monitoring and Management, Diagnostics 2014, 4 (3), 104-128; mReader from Mobile Assay; and Holomic Rapid Diagnostic Test Reader).

As noted herein, certain embodiments allow detection via colorimetric change which has certain attendant benefits when embodiments are utilized in POC situations and or in resource poor environments where access to more complex detection equipment to readout the signal may be limited. However, portable embodiments disclosed herein may also be coupled with hand-held spectrophotometers that enable detection of signals outside the visible range. An example of a hand-held spectrophotometer device that may be used in combination with the present invention is described in Das et al. “Ultra-portable, wireless smartphone spectrophotometer for rapid, non-destructive testing of fruit ripeness.” Nature Scientific Reports. 2016, 6:32504, DOI: 10.1038/srep32504. Finally, in certain embodiments utilizing quantum dot-based detection constructs, use of a handheld UV light, or other suitable device, may be successfully used to detect a signal owing to the near complete quantum yield provided by quantum dots.

### Spatial Detection

In an embodiment, the method of multiomic analysis described herein can include spatial detection of genomic, epigenomic, transcriptomic, and/or proteomic information of a population of cells, tissues and/or organisms. In an embodiment, one or more oligonucleotide-adorned beads are present on a surface of the substrate or container and are arranged in an ordered array, wherein each oligonucleotide-adorned bead has a unique barcode corresponding to the x,y coordinate of the oligonucleotide-adorned bead in the array. In an embodiment, the method further includes depositing a tissue section comprising the one or more individual cells on the ordered array. In an embodiment, the one or more individual cells are present in a tissue sample and specific binding and fixing occurs in situ. In an embodiment, sequencing the genetically encoded affinity molecule, the genetically encoded sequencing molecule, or both and sequencing the one or more cellular polynucleotides, one or more nuclear polynucleotides, or both occurs in situ.

### Methods of Using the Cis Regulatory Elements

Methods of Specific Detection of Cell Type, Cell State, Tissue Type, and/or Environment

Described in certain example embodiments herein are methods of detecting a specific cell type, cell state, tissue type, and/or environment of one or more cells in a sample comprising delivering to one or more cells an engineered reporter polynucleotide of the present invention, a vector or vector system comprising the same, and/or a delivery vehicle comprising the same under conditions sufficient for expression of the engineered reporter polynucleotide, wherein expression of the reporter polynucleotide occurs substantially only in the specific cell type, cell state, tissue type, and/or environment in which the CRE is active in. Exemplary cell types, states, tissue types, and environmental conditions are discussed elsewhere herein.

In certain example embodiments, expression of the reporter polynucleotide generates a detectable signal. In certain example embodiments, the method further includes contacting the one or more cells with a detection reagent, wherein the detection reagent comprises a sequence-specific binding molecule or system capable of specifically binding the reporter polynucleotide, optionally at the target sequence for a sequence-specific binding molecule or system.

In an embodiment, the sequence-specific binding molecule or system comprises a programmable nuclease or system thereof, optionally wherein the programmable nuclease or system thereof is a Cas or Cas-based system, an IscB or IscB system, or an OMEGA system.

In an embodiment, binding of the sequence-specific binding molecule or system to specifically binding the reporter polynucleotide produces a detectable signal. In an embodiment, the method further comprises detecting the detectable signal. In an embodiment, the detectable signal indicates a specific cell type, cell state, tissue type, and/or environment. In some embodiment, the detectable signal is increased in the specific cell type, cell state, tissue type, and/or environment in which the one or more CREs are active in as compared to cells, tissues, or environments that the CREs are not active in. In some embodiment, the detectable signal is decreased in the specific cell type, cell state, tissue type, and/or environment in which the one or more CREs are active in as compared to cells, tissues, or environments that the CREs are not active in.

In an embodiment, the detectable signal is an optical signal, a genetic perturbation, a change in gene expression of a target gene, expression of a barcode, change in genotype, change in phenotype, or any combination thereof.

In an embodiment, detection comprises optical detection of the detectable signal, DNA sequencing, RNA sequencing, a hybridization-based gene expression analysis, mass-spectrometry, immunodetection, or any combination thereof.

In an embodiment, detection comprises a single-cell resolved assay. Exemplary single-cell resolved assays include any of those described in e.g., Wen and Tang, Precision Clinical Medicine, 2022, 5: pbac002.

In an embodiment, the sample comprises a biofluid optionally selected from saliva, urine, blood or portion thereof, sweat, milk, semen, lymph, mucus, or feces. In an embodiment, the sample comprises a tissue or portion thereof. Other suitable samples are described elsewhere herein, such as e.g., in connection with the devices of the present invention.

In an embodiment, the method comprises in situ spatial detection of expression of the reporter polynucleotide. In an embodiment, the method comprises delivering multiple engineered reporter polynucleotides with different CREs that are active in different cell types such that when used in connection with an in situ spatial detection method, the spatial organization of the cell types, states, etc. within the tissue can be resolved.

In an embodiment, one or more of the steps of the method are performed in vitro, in vivo, in situ, or ex vivo.

### Methods of Specific Delivery of Therapeutic Polynucleotides

As previously discussed, the CREs of the present invention can be leveraged to provide cell type, cell state, tissue type, and/or environment specific delivery/expression of one or more of the therapeutic polynucleotides. In this way, cell type, cell state, tissue type, and/or environment specific treatment of a disease can be achieved.

### Methods of Treatment

In an embodiment, the disease to be treated by one or more engineered therapeutic polynucleotides can be any disease, including but not limited to a genetic disease or disorder, non-genetic disease or disorder or disease caused by infection by a microorganism or virus. Treating Diseases of the Circulatory System

In an embodiment, an engineered therapeutic polynucleotide of the present invention described herein can be used to treat and/or prevent a circulatory system disease. Exemplary diseases are provided, for example, in Tables 4 and 5 as well as a disease identified as being caused or attributed to a mtDNA mutation set forth at mitomap.org. In an embodiment the plasma exosomes of Wahlgren et al. (Nucleic Acids Research, 2012, Vol. 40, No. 17 e130) can be used to deliver the engineered therapeutic polynucleotide of the present invention (e.g., such as one containing a genetic modification system, such as a CRISPR-Cas system, and/or component thereof described herein) to the blood. In an embodiment, the circulatory system disease can be treated by using a lentivirus to deliver the engineered therapeutic polynucleotide of the present invention to modify or treat hematopoietic stem cells (HSCs) in vivo or ex vivo (see e.g. Drakopoulou, “Review Article, The Ongoing Challenge of Hematopoietic Stem Cell-Based Gene Therapy for β-Thalassemia,” Stem Cells International, Volume 2011, Article ID 987980, 10 pages, doi: 10.4061/2011/987980, which can be adapted for use with the engineered therapeutic polynucleotide of the present invention in view of the description herein). In an embodiment, the circulatory system disorder can be treated by correcting HSCs as to the disease using a engineered therapeutic polynucleotide of the present invention or a component thereof, wherein the engineered therapeutic polynucleotide of the present invention optionally comprises a CRISPR-Cas system that optionally includes a suitable HDR repair template (see e.g. Cavazzana, “Outcomes of Gene Therapy for β-Thalassemia Major via Transplantation of Autologous Hematopoietic Stem Cells Transduced Ex Vivo with a Lentiviral BA-T87Q-Globin Vector.”; Cavazzana-Calvo, “Transfusion independence and HMGA2 activation after gene therapy of human β-thalassaemia”, Nature 467, 318-322 (16 Sep. 2010) doi: 10.1038/nature09328; Nienhuis, “Development of Gene Therapy for Thalassemia, Cold Spring Harbor Perspectives in Medicine, doi: 10.1101/cshperspect.a011833 (2012), LentiGlobin BB305, a lentiviral vector containing an engineered β-globin gene (BA-T87Q); and Xie et al., “Seamless gene correction of β-thalassaemia mutations in patient-specific iPSCs using CRISPR/Cas9 and piggyback” Genome Research gr. 173427.114 (2014) genome.org/cgi/doi/10.1101/gr.173427.114 (Cold Spring Harbor Laboratory Press;

Watts, “Hematopoietic Stem Cell Expansion and Gene Therapy” Cytotherapy 13 (10): 1164-1171. doi: 10.3109/14653249.2011.620748 (2011), which can be adapted for use with the CRISPR-Cas systems herein in view of the description herein). In an embodiment, iPSCs can be modified using a engineered therapeutic polynucleotide of the present invention described herein to correct a disease polynucleotide associated with a circulatory disease. In this regard, the teachings of Xu et al. (Sci Rep. 2015 Jul. 9; 5:12065. doi: 10.1038/srep12065) and Song et al. (Stem Cells Dev. 2015 May 1; 24 (9): 1053-65. doi: 10.1089/scd.2014.0347. Epub 2015 Feb. 5) with respect to modifying iPSCs can be adapted for use in view of the description herein with engineered therapeutic polynucleotide of the present invention. In an embodiment, the engineered therapeutic polynucleotide of the present invention comprises a polynucleotide encoding a genetic modifying system or component(s) thereof.

The term “Hematopoietic Stem Cell” or “HSC” refers broadly those cells considered to be an HSC, e.g., blood cells that give rise to all the other blood cells and are derived from mesoderm; located in the red bone marrow, which is contained in the core of most bones. HSCs of the invention include cells having a phenotype of hematopoietic stem cells, identified by small size, lack of lineage (lin) markers, and markers that belong to the cluster of differentiation series, like: CD34, CD38, CD90, CD133, CD105, CD45, and also c-kit, —the receptor for stem cell factor. Hematopoietic stem cells are negative for the markers that are used for detection of lineage commitment, and are, thus, called Lin-; and, during their purification by FACS, a number of up to 14 different mature blood-lineage markers, e.g., CD13 & CD33 for myeloid, CD71 for erythroid, CD19 for B cells, CD61 for megakaryocytic, etc. for humans; and, B220 (murine CD45) for B cells, Mac-1 (CD11b/CD18) for monocytes, Gr-1 for Granulocytes, Ter119 for erythroid cells, I17Ra, CD3, CD4, CD5, CD8 for T cells, etc. Mouse HSC markers: CD34lo/−, SCA-1+, Thyl.1+/lo, CD38+, C-kit+, lin-, and Human HSC markers: CD34+, CD59+, Thyl/CD90+, CD38lo/−, C-kit/CD117+, and lin-. HSCs are identified by markers. Hence in embodiments discussed herein, the HSCs can be CD34+ cells. HSCs can also be hematopoietic stem cells that are CD34−/CD38−. Stem cells that may lack c-kit on the cell surface that are considered in the art as HSCs are within the ambit of the invention, as well as CD133+ cells likewise considered HSCs in the art.

In an embodiment, the treatment or prevention for treating a circulatory system or blood disease can include modifying a human cord blood cell with any modification described herein using an engineered therapeutic polynucleotide of the present invention. In an embodiment, the treatment or prevention for treating a circulatory system or blood disease can include modifying a granulocyte colony-stimulating factor-mobilized peripheral blood cell (mPB) with any modification described herein. In an embodiment, the human cord blood cell or mPB can be CD34+. In an embodiment, the cord blood cell(s) or mPB cell(s) modified can be autologous. In an embodiment, the cord blood cell(s) or mPB cell(s) can be allogenic. In addition to the modification of the disease gene(s), allogenic cells can be further modified using the composition, system, described herein to reduce the immunogenicity of the cells when delivered to the recipient. Such techniques are described elsewhere herein and e.g. Cartier, “MINI-SYMPOSIUM: X-Linked Adrenoleukodystrophypa, Hematopoietic Stem Cell Transplantation and Hematopoietic Stem Cell Gene Therapy in X-Linked Adrenoleukodystrophy,” Brain Pathology 20 (2010) 857-862, which can be adapted for use with the composition, system, herein. The modified cord blood cell(s) or mPB cell(s) can be optionally expanded in vitro. The modified cord blood cell(s) or mPB cell(s) can be derived to a subject in need thereof using any suitable delivery technique.

The engineered therapeutic polynucleotide of the present invention can contain a genetic modifying agent (such as a CRISPR-Cas system) to target genetic locus or loci in HSCs. In an embodiment, the Cas effector(s) can be codon-optimized for a eukaryotic cell and especially a mammalian cell, e.g., a human cell, for instance, HSC, or iPSC and sgRNA targeting a locus or loci in HSC, such as circulatory disease, can be prepared. These may be delivered via particles. The particles may be formed by the Cas effector (e.g., Cas9) protein and the gRNA being admixed. The gRNA and Cas effector (e.g., Cas9) protein mixture can be, for example, admixed with a mixture comprising or consisting essentially of or consisting of surfactant, phospholipid, biodegradable polymer, lipoprotein and alcohol, whereby particles containing the gRNA and Cas effector (e.g. Cas9) protein may be formed. The invention comprehends so making particles and particles from such a method as well as uses thereof. Particles can be used to deliver the engineered therapeutic polynucleotide of the present invention to blood or circulatory system.

In an embodiment, after ex vivo modification the HSCs or iPCS can be expanded prior to administration to the subject. Expansion of HSCs can be via any suitable method such as that described by, Lee, “Improved ex vivo expansion of adult hematopoietic stem cells by overcoming CUL4-mediated degradation of HOXB4.” Blood. 2013 May 16; 121(20): 4082-9. doi: 10.1182/blood-2012-09-455204. Epub 2013 Mar. 21.

In an embodiment, the HSCs or iPSCs modified can be autologous. In an embodiment, the HSCs or iPSCs can be allogenic. In addition to the modification of the disease gene(s), allogenic cells can be further modified using the engineered therapeutic polynucleotide of the present invention (such as one containing a genetic modifying agent or component(s) thereof) described herein to reduce the immunogenicity of the cells when delivered to the recipient. Such techniques are described elsewhere herein and e.g. Cartier, “MINI-SYMPOSIUM: X-Linked Adrenoleukodystrophypa, Hematopoietic Stem Cell Transplantation and Hematopoietic Stem Cell Gene Therapy in X-Linked Adrenoleukodystrophy,” Brain Pathology 20 (2010) 857-862, which can be adapted for use with the CRISPR-Cas system herein.

### Treating Diseases of the Brain

In an embodiment, the engineered therapeutic polynucleotide of the present invention are used to treat diseases of the brain and CNS. Delivery options for the brain include encapsulation of an engineered therapeutic polynucleotide of the present invention into liposomes and conjugating to molecular Trojan horses for trans-blood brain barrier (BBB) delivery. In an embodiment, the engineered therapeutic polynucleotide of the present invention encodes a CRISPR-Cas enzyme and guide RNA in the form of either DNA or RNA Molecular Trojan horses have been shown to be effective for delivery of B-gal expression vectors into the brain of non-human primates. The same approach can be used to delivery vectors containing CRISPR enzyme (e.g., a Cas) and guide RNA. For instance, Xia CF and Boado R J, Pardridge W M (“Antibody-mediated targeting of siRNA via the human insulin receptor using avidin-biotin technology.” Mol Pharm. 2009 May-June; 6 (3): 747-51. doi: 10.1021/mp800194) describes how delivery of short interfering RNA (siRNA) to cells in culture, and in vivo, is possible with combined use of a receptor-specific monoclonal antibody (mAb) and avidin-biotin technology. The authors also report that because the bond between the targeting mAb and the siRNA is stable with avidin-biotin technology, and RNAi effects at distant sites such as brain are observed in vivo following an intravenous administration of the targeted siRNA, the teachings of which can be adapted for use with the engineered therapeutic polynucleotide of the present invention, such as those containing a genetic modifying agent such as a CRISPR-Cas systm. In other embodiments, an artificial virus can be generated for CNS and/or brain delivery. See e.g. Zhang et al. (Mol Ther. 2003 January; 7 (1): 11-8.)), the teachings of which can be adapted for use with the CRISPR-Cas systems herein.

### Treating Hearing Diseases

In an embodiment the engineered therapeutic polynucleotide of the present invention described herein can be used to treat a hearing disease or hearing loss in one or both ears. Deafness is often caused by lost or damaged hair cells that cannot relay signals to auditory neurons. In such cases, cochlear implants may be used to respond to sound and transmit electrical signals to the nerve cells. But these neurons often degenerate and retract from the cochlea as fewer growth factors are released by impaired hair cells.

In an embodiment, the engineered therapeutic polynucleotides of the present invention or modified cells can be delivered to one or both ears for treating or preventing hearing disease or loss by any suitable method or technique. Suitable methods and techniques include, but are not limited to, those set forth in U.S. patent application No. 20120328580 describes injection of a pharmaceutical composition into the ear (e.g., auricular administration), such as into the luminae of the cochlea (e.g., the Scala media, Sc vestibulae, and Sc tympani), e.g., using a syringe, e.g., a single-dose syringe. For example, one or more of the compounds described herein can be administered by intratympanic injection (e.g., into the middle ear), and/or injections into the outer, middle, and/or inner ear; administration in situ, via a catheter or pump (see e.g. McKenna et al., (U.S. Publication No. 2006/0030837) and Jacobsen et al., (U.S. Pat. No. 7,206,639); administration in combination with a mechanical device such as a cochlear implant or a hearing aid, which is worn in the outer ear (see e.g. U.S. Publication No. 2007/0093878, which provides an exemplary cochlear implant suitable for delivery of the the engineered therapeutic polynucleotide of the present invention described herein to the ear). Such methods are routinely used in the art, for example, for the administration of steroids and antibiotics into human ears. Injection can be, for example, through the round window of the ear or through the cochlear capsule. Other inner ear administration methods are known in the art (see, e.g., Salt and Plontke, Drug Discovery Today, 10:1299-1306, 2005). In an embodiment, a catheter or pump can be positioned, e.g., in the ear (e.g., the outer, middle, and/or inner ear) of a patient during a surgical procedure. In an embodiment, a catheter or pump can be positioned, e.g., in the ear (e.g., the outer, middle, and/or inner ear) of a patient without the need for a surgical procedure.

In general, the cell therapy methods described in U.S. patent application 20120328580 can be used to promote complete or partial differentiation of a cell to or towards a mature cell type of the inner ear (e.g., a hair cell) in vitro. Cells resulting from such methods can then be transplanted or implanted into a patient in need of such treatment. The cell culture methods required to practice these methods, including methods for identifying and selecting suitable cell types, methods for promoting complete or partial differentiation of selected cells, methods for identifying complete or partially differentiated cell types, and methods for implanting complete or partially differentiated cells are described below.

Cells suitable for use with the present invention include and/or are in need of treatment, but are not limited to, cells that are capable of differentiating completely or partially into a mature cell of the inner ear, e.g., a hair cell (e.g., an inner and/or outer hair cell), when contacted, e.g., in vitro, with one or more of the compounds described herein. Exemplary cells that are capable of differentiating into a hair cell include, but are not limited to stem cells (e.g., inner ear stem cells, adult stem cells, bone marrow derived stem cells, embryonic stem cells, mesenchymal stem cells, skin stem cells, iPS cells, and fat derived stem cells), progenitor cells (e.g., inner ear progenitor cells), support cells (e.g., Deiters' cells, pillar cells, inner phalangeal cells, tectal cells and Hensen's cells), and/or germ cells. The use of stem cells for the replacement of inner ear sensory cells is described in Li et al., (U.S. Publication No. 2005/0287127) and Li et al., (U.S. patent Ser. No. 11/953,797). The use of bone marrow derived stem cells for the replacement of inner ear sensory cells is described in Edge et al., PCT/US2007/084654. iPS cells are described, e.g., at Takahashi et al., Cell, Volume 131, Issue 5, Pages 861-872 (2007); Takahashi and Yamanaka, Cell 126, 663-76 (2006); Okita et al., Nature 448, 260-262 (2007); Yu, J. et al., Science 318 (5858): 1917-1920 (2007); Nakagawa et al., Nat. Biotechnol. 26:101-106 (2008); and Zaehres and Scholer, Cell 131 (5): 834-835 (2007). Such suitable cells can be identified by analyzing (e.g., qualitatively or quantitatively) the presence of one or more tissue specific genes. For example, gene expression can be detected by detecting the protein product of one or more tissue-specific genes. Protein detection techniques involve staining proteins (e.g., using cell extracts or whole cells) using antibodies against the appropriate antigen. In this case, the appropriate antigen is the protein product of the tissue-specific gene expression. Although, in principle, a first antibody (i.e., the antibody that binds the antigen) can be labeled, it is more common (and improves the visualization) to use a second antibody directed against the first (e.g., an anti-IgG). This second antibody is conjugated either with fluorochromes, or appropriate enzymes for colorimetric reactions, or gold beads (for electron microscopy), or with the biotin-avidin system, so that the location of the primary antibody, and thus the antigen, can be recognized.

The engineered therapeutic polynucleotide of the present invention may be delivered to the ear by direct application of pharmaceutical composition to the outer ear, with compositions modified from US Published application, 20110142917. In an embodiment the pharmaceutical composition is applied to the ear canal. Delivery to the ear may also be referred to as aural or otic delivery.

In an embodiment, the engineered therapeutic polynucleotide of the present invention and/or vectors or vector systems can be delivered to ear via a transfection to the inner ear through the intact round window by a novel proteidic delivery technology which may be applied to the nucleic acid-targeting system of the present invention (see, e.g., Qi et al., Gene Therapy (2013), 1-9). About 40 μl of 10 mM RNA may be contemplated as the dosage for administration to the ear.

According to Rejali et al. (Hear Res. 2007 June; 228 (1-2): 180-7), cochlear implant function can be improved by good preservation of the spiral ganglion neurons, which are the target of electrical stimulation by the implant and brain derived neurotrophic factor (BDNF) has previously been shown to enhance spiral ganglion survival in experimentally deafened ears. Rejali et al. tested a modified design of the cochlear implant electrode that includes a coating of fibroblast cells transduced by a viral vector with a BDNF gene insert. To accomplish this type of ex vivo gene transfer, Rejali et al. transduced guinea pig fibroblasts with an adenovirus with a BDNF gene cassette insert, and determined that these cells secreted BDNF and then attached BDNF-secreting cells to the cochlear implant electrode via an agarose gel, and implanted the electrode in the scala tympani. Rejali et al. determined that the BDNF expressing electrodes were able to preserve significantly more spiral ganglion neurons in the basal turns of the cochlea after 48 days of implantation when compared to control electrodes and demonstrated the feasibility of combining cochlear implant therapy with ex vivo gene transfer for enhancing spiral ganglion neuron survival. Such a system may be applied to the nucleic acid-targeting system of the present invention for delivery to the ear.

In an embodiment, the system set forth in Mukherjea et al. (Antioxidants & Redox Signaling, Volume 13, Number 5, 2010) can be adapted for transtympanic administration of the the engineered therapeutic polynucleotide of the present invention thereof to the ear. In an embodiment, a dosage of about 2 mg to about 4 mg of the engineered therapeutic polynucleotide of the present invention for administration to a human.

In an embodiment, the system set forth in [Jung et al. (Molecular Therapy, vol. 21 no. 4, 834-841 Apr. 2013) can be adapted for vestibular epithelial delivery of the the engineered therapeutic polynucleotide of the present invention to the ear. In an embodiment, a dosage of about 1 to about 30 mg of the engineered therapeutic polynucleotide of the present invention for administration to a human.

### Treating Diseases in Non-Dividing Cells

In an embodiment, a gene or transcript to be corrected is in a non-dividing cell. Exemplary non-dividing cells are muscle cells or neurons. Non-dividing (especially non-dividing, fully differentiated) cell types present issues for gene targeting or genome engineering, for example because homologous recombination (HR) is generally suppressed in the G1 cell-cycle phase. However, while studying the mechanisms by which cells control normal DNA repair systems, Durocher discovered a previously unknown switch that keeps HR “off” in non-dividing cells and devised a strategy to toggle this switch back on. Orthwein et al. (Daniel Durocher's lab at the Mount Sinai Hospital in Ottawa, Canada) recently reported (Nature 16142, published online 9 Dec. 2015) have shown that the suppression of HR can be lifted and gene targeting successfully concluded in both kidney (293T) and osteosarcoma (U2OS) cells. Tumor suppressors, BRCA1, PALB2 and BRAC2 are known to promote DNA DSB repair by HR. They found that formation of a complex of BRCA1 with PALB2-BRAC2 is governed by a ubiquitin site on PALB2, such that action on the site by an E3 ubiquitin ligase. This E3 ubiquitin ligase is composed of KEAP1 (a PALB2-interacting protein) in complex with cullin-3 (CUL3)-RBX1. PALB2 ubiquitylation suppresses its interaction with BRCA1 and is counteracted by the deubiquitylase USP11, which is itself under cell cycle control. Restoration of the BRCA1-PALB2 interaction combined with the activation of DNA-end resection is sufficient to induce homologous recombination in G1, as measured by a number of methods including a CRISPR-Cas9-based gene-targeting assay directed at USP11 or KEAP1 (expressed from a pX459 vector). However, when the BRCA1-PALB2 interaction was restored in resection-competent G1 cells using either KEAP1 depletion or expression of the PALB2-KR mutant, a robust increase in gene-targeting events was detected. These teachings can be adapted for use and/or applied to the engineered therapeutic polynucleotides of the present invention described herein.

Thus, reactivation of HR in cells, especially non-dividing, fully differentiated cell types is preferred, In an embodiment. In an embodiment, promotion of the BRCA1-PALB2 interaction is preferred In an embodiment. In an embodiment, the target ell is a non-dividing cell. In an embodiment, the target cell is a neuron or muscle cell. In an embodiment, the target cell is targeted in vivo. In an embodiment, the cell is in G1 and HR is suppressed. In an embodiment, use of KEAP1 depletion, for example inhibition of expression of KEAP1 activity, is preferred. KEAP1 depletion may be achieved through siRNA, for example as shown in Orthwein et al. Alternatively, expression of the PALB2-KR mutant (lacking all eight Lys residues in the BRCA1-interaction domain is preferred, either in combination with KEAP1 depletion or alone. PALB2-KR interacts with BRCA1 irrespective of cell cycle position. Thus, promotion or restoration of the BRCA1-PALB2 interaction, especially in G1 cells, is preferred In an embodiment, especially where the target cells are non-dividing, or where removal and return (ex vivo gene targeting) is problematic, for example neuron or muscle cells. KEAP1 siRNA is available from ThermoFischer. In an embodiment, a BRCA1-PALB2 complex may be delivered to the G1 cell. In an embodiment, PALB2 deubiquitylation may be promoted for example by increased expression of the deubiquitylase USP11, so it is envisaged that a construct may be provided to promote or up-regulate expression or activity of the deubiquitylase USP11.

### Treating Diseases of the Eye

In an embodiment, the disease to be treated is a disease that affects the eyes. Thus, In an embodiment, the engineered therapeutic polynucleotide of the present invention is delivered to one or both eyes.

The engineered therapeutic polynucleotide of the present invention can be used to correct ocular defects that arise from several genetic mutations further described in Genetic Diseases of the Eye, Second Edition, edited by Elias I. Traboulsi, Oxford University Press, 2012.

In an embodiment, the condition to be treated or targeted is an eye disorder. In an embodiment, the eye disorder may include glaucoma. In an embodiment, the eye disorder includes a retinal degenerative disease. In an embodiment, the retinal degenerative disease is selected from Stargardt disease, Bardet-Biedl Syndrome, Best disease, Blue Cone Monochromacy, Choroidermia, Cone-rod dystrophy, Congenital Stationary Night Blindness, Enhanced S-Cone Syndrome, Juvenile X-Linked Retinoschisis, Leber Congenital Amaurosis, Malattia Leventinesse, Norrie Disease or X-linked Familial Exudative Vitreoretinopathy, Pattern Dystrophy, Sorsby Dystrophy, Usher Syndrome, Retinitis Pigmentosa, Achromatopsia or Macular dystrophies or degeneration, Retinitis Pigmentosa, Achromatopsia, and age related macular degeneration. In an embodiment, the retinal degenerative disease is Leber Congenital Amaurosis (LCA) or Retinitis Pigmentosa. Other exemplary eye diseases are described in greater detail elsewhere herein.

In an embodiment, the engineered therapeutic polynucleotide of the present invention is delivered to the eye, optionally via intravitreal injection or subretinal injection. Intraocular injections may be performed with the aid of an operating microscope. For subretinal and intravitreal injections, eyes may be prolapsed by gentle digital pressure and fundi visualized using a contact lens system consisting of a drop of a coupling medium solution on the cornea covered with a glass microscope slide coverslip. For subretinal injections, the tip of a 10-mm 34-gauge needle, mounted on a 5-μl Hamilton syringe may be advanced under direct visualization through the superior equatorial sclera tangentially towards the posterior pole until the aperture of the needle was visible in the subretinal space. Then, 2 μl of vector suspension may be injected to produce a superior bullous retinal detachment, thus confirming subretinal vector administration. This approach creates a self-sealing sclerotomy allowing the vector suspension to be retained in the subretinal space until it is absorbed by the RPE, usually within 48 h of the procedure. This procedure may be repeated in the inferior hemisphere to produce an inferior retinal detachment. This technique results in the exposure of approximately 70% of neurosensory retina and RPE to the vector suspension. For intravitreal injections, the needle tip may be advanced through the sclera 1 mm posterior to the corneoscleral limbus and 2 μl of vector suspension injected into the vitreous cavity. For intracameral injections, the needle tip may be advanced through a corneoscleral limbal paracentesis, directed towards the central cornea, and 2 μl of vector suspension may be injected. For intracameral injections, the needle tip may be advanced through a corneoscleral limbal paracentesis, directed towards the central cornea, and 2 μl of vector suspension may be injected. These vectors may be injected at titers of either 1.0-1.4×10¹⁰ or 1.0-1.4×10⁹ transducing units (TU)/ml.

In an embodiment, for administration to the eye, lentiviral vectors. In an embodiment, the lentiviral vector is an equine infectious anemia virus (EIAV) vector. Exemplary EIAV vectors for eye delivery are described in Balagaan, J Gene Med 2006; 8:275-285, Published online 21 Nov. 2005 in Wiley InterScience (interscience.wiley.com). DOI: 10.1002/jgm.845; Binley et al., HUMAN GENE THERAPY 23:980-991 (September 2012), which can be adapted for use with the engineered therapeutic polynucleotides of the present invention. In an embodiment, the dosage can be 1.1×10⁵ transducing units per eye (TU/eye) in a total volume of 100 μl.

Other viral vectors can also be used for delivery to the eye, such as AAV vectors, such as those described in Campochiaro et al., Human Gene Therapy 17:167-176 (February 2006), Millington-Ward et al. (Molecular Therapy, vol. 19 no. 4, 642-649 April 2011; Dalkara et al. (Sci Transl Med 5, 189ra76 (2013)), which can be adapted for use with the engineered therapeutic polynucleotides of the present invention. In an embodiment, the dose can range from about 10⁶ to 10^{9.5} particle units. In the context of the Millington-Ward AAV vectors, a dose of about 2×10¹¹ to about 6×10¹³ virus particles can be administered. In the context of Dalkara vectors, a dose of about 1×10¹⁵ to about 1×10¹⁶ vg/ml administered to a human.

In an embodiment, the Sd-rxRNA® system of RXi Pharmaceuticals may be used/and or adapted for delivering the engineered therapeutic polynucleotides of the present invention to the eye. In this system, a single intravitreal administration of 3 μg of sd-rxRNA results in sequence-specific reduction of PPIB mRNA levels for 14 days. The sd-rxRNA® system may be applied to the nucleic acid-targeting system of the present invention, contemplating a dose of about 3 to 20 mg of CRISPR administered to a human.

In other embodiments, the methods of US Patent Publication No. 20130183282, which is directed to methods of cleaving a target sequence from the human rhodopsin gene, may also be modified to the nucleic acid-targeting system of the present invention.

In other embodiments, the methods of US Patent Publication No. 20130202678 for treating retinopathies and sight-threatening ophthalmologic disorders relating to delivering of the Puf-A gene (which is expressed in retinal ganglion and pigmented cells of eye tissues and displays a unique anti-apoptotic activity) to the sub-retinal or intravitreal space in the eye. In particular, desirable targets are zgc: 193933, prdmla, spata2, tex10, rbb4, ddx3, zp2.2, Blimp-1 and HtrA2, all of which may be targeted by the CRISPR-Cas system of the present invention.

Wu (Cell Stem Cell, 13:659-62, 2013) designed a guide RNA that led Cas9 to a single base pair mutation that causes cataracts in mice, where it induced DNA cleavage. Then using either the other wild-type allele or oligos given to the zygotes repair mechanisms corrected the sequence of the broken allele and corrected the cataract-causing genetic defect in mutant mouse. This approach can be adapted to and/or applied to the engineered therapeutic polynucleotides of the present invention.

US Patent Publication No. 20120159653, describes use of zinc finger nucleases to genetically modify cells, animals and proteins associated with macular degeneration (MD), the teachings of which can be applied to and/or adapted for the CRISPR-Cas systems described herein.

One aspect of US Patent Publication No. 20120159653 relates to editing of any chromosomal sequences that encode proteins associated with MD which may be applied to the nucleic acid-targeting system of the present invention.

### Treating Muscle Diseases and Cardiovascular Diseases

In an embodiment, the engineered therapeutic polynucleotides of the present invention can be used to treat and/or prevent a muscle disease and associated circulatory or cardiovascular disease or disorder. The present invention also contemplates a genetic modifying agent, gene therapy, protein therapy, or other therapeutic polynucleotide or gene product produced therefrom, to the heart. For the heart, a myocardium tropic adeno-associated virus (AAVM) is preferred, in particular AAVM41 which showed preferential gene transfer in the heart (see, e.g., Lin-Yanga et al., PNAS, Mar. 10, 2009, vol. 106, no. 10). Administration may be systemic or local. A dosage of about 1-10×10¹⁴ vector genomes is contemplated for systemic administration. See also, e.g., Eulalio et al. (2012) Nature 492:376 and Somasuntharam et al. (2013) Biomaterials 34:7790, the teachings of which can be adapted for and/or applied to the engineered therapeutic polynucleotides of the present invention described herein.

For example, US Patent Publication No. 20110023139, the teachings of which can be adapted for and/or applied to the engineered therapeutic polynucleotides of the present invention, describes use of zinc finger nucleases to genetically modify cells, animals and proteins associated with cardiovascular disease. Cardiovascular diseases generally include high blood pressure, heart attacks, heart failure, and stroke and TIA. Any chromosomal sequence involved in cardiovascular disease or the protein encoded by any chromosomal sequence involved in cardiovascular disease may be utilized in the methods described in this disclosure. The cardiovascular-related proteins are typically selected based on an experimental association of the cardiovascular-related protein to the development of cardiovascular disease. For example, the production rate or circulating concentration of a cardiovascular-related protein may be elevated or depressed in a population having a cardiovascular disorder relative to a population lacking the cardiovascular disorder. Differences in protein levels may be assessed using proteomic techniques including but not limited to Western blot, immunohistochemical staining, enzyme linked immunosorbent assay (ELISA), and mass spectrometry. Alternatively, the cardiovascular-related proteins may be identified by obtaining gene expression profiles of the genes encoding the proteins using genomic techniques including but not limited to DNA microarray analysis, serial analysis of gene expression (SAGE), and quantitative real-time polymerase chain reaction (Q-PCR). Exemplary chromosomal sequences can be found in Table 5.

The engineered therapeutic polynucleotides of the present invention can be used for treating diseases of the muscular system. The present invention also contemplates delivering the engineered therapeutic polynucleotides of the present invention to muscle(s). In an embodiment, the muscle is smooth muscle, cardiac muscle, and/or skeletal muscle.

In an embodiment, the muscle disease to be treated is a muscle dystrophy such as DMD. In an embodiment, the engineered therapeutic polynucleotides of the present invention comprises a polynucleotide encoding a genetic modification system, such as a system capable of RNA modification, which can be used to achieve exon skipping to achieve correction of the diseased gene. In an embodiment, the genetic modification system included or encoded by the therapeutic polynucleotide is a CRISPR-Cas system. As used herein, the term “exon skipping” refers to the modification of pre-mRNA splicing by the targeting of splice donor and/or acceptor sites within a pre-mRNA with one or more complementary antisense oligonucleotide(s) (AONs). By blocking access of a spliceosome to one or more splice donor or acceptor site, an AON may prevent a splicing reaction thereby causing the deletion of one or more exons from a fully-processed mRNA. Exon skipping may be achieved in the nucleus during the maturation process of pre-mRNAs. In some examples, exon skipping may include the masking of key sequences involved in the splicing of targeted exons by using a genetic modifying system (e.g., a CRISPR-Cas system) described herein capable of RNA modification. In an embodiment, exon skipping can be achieved in dystrophin mRNA. In an embodiment, the engineered therapeutic polynucleotides of the present invention (e.g., one comprising or encoding a CRISPR-Cas system or component(s) thereof) can induce exon skipping at exon 1, 2, 3, 4, 5, 6, 7, 8, 10, 11, 12, 13, 14, 15, 16, 17, 18, 19, 20, 21, 22, 23, 24, 25, 26, 27, 28, 29, 30, 31, 32, 33, 34, 45, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or any combination thereof of the dystrophin mRNA. In an embodiment, the engineered therapeutic polynucleotides of the present invention (e.g., one comprising or encoding a CRISPR-Cas system or component(s) thereof) can induce exon skipping at exon 43, 44, 50, 51, 52, 55, or any combination thereof of the dystrophin mRNA. Mutations in these exons, can also be corrected using non-exon skipping polynucleotide modification methods.

In an embodiment, for treatment of a muscle disease, the method of Bortolanza et al. Molecular Therapy vol. 19 no. 11, 2055-264 Nov. 2011) may be applied to an AAV expressing CRISPR Cas and injected into humans at a dosage of about 2 ×10¹⁵ or 2×10¹⁶ vg of vector. The teachings of Bortolanza et al., can be adapted for and/or applied to the engineered therapeutic polynucleotides of the present invention described herein.

In an embodiment, the method of Dumonceaux et al. (Molecular Therapy vol. 18 no. 5, 881-887 May 2010) may be applied to an AAV expressing CRISPR Cas and injected into humans, for example, at a dosage of about 1014 to about 10¹⁵ vg of vector. The teachings of Dumonceaux described herein can be adapted for and/or applied to the engineered therapeutic polynucleotides of the present invention described herein.

In an embodiment, the method of Kinouchi et al. (Gene Therapy (2008) 15, 1126-1130) may be applied to the engineered therapeutic polynucleotides of the present invention and injected into a human, for example, at a dosage of about 500 to 1000 ml of a 40 μM solution into the muscle.

In an embodiment, the method of Hagstrom et al. (Molecular Therapy Vol. 10, No. 2, August 2004) can be adapted for and/or applied to the engineered therapeutic polynucleotides of the present invention and injected at a dose of about 15 to about 50 mg into the great saphenous vein of a human.

### Treating Diseases of the Liver and Kidney

In an embodiment, the engineered therapeutic polynucleotides of the present invention described herein can be used to treat a disease of the kidney or liver. Thus, In an embodiment, delivery and/or expression of the engineered therapeutic polynucleotides of the present invention is to or in the liver or kidney.

Delivery strategies to induce cellular uptake of the therapeutic nucleic acid include physical force or vector systems such as viral-, lipid- or complex-based delivery, or nanocarriers. From the initial applications with less possible clinical relevance, when nucleic acids were addressed to renal cells with hydrodynamic high-pressure injection systemically, a wide range of gene therapeutic viral and non-viral carriers have been applied already to target posttranscriptional events in different animal kidney disease models in vivo (Csaba Révész and Peter Hamar (2011). Delivery Methods to Target RNAs in the Kidney, Gene Therapy Applications, Prof. Chunsheng Kang (Ed.), ISBN: 978-953-307-541-9, InTech, Available from: intechopen.com/books/gene-therapy-applications/delivery-methods-to-target-rnas-inthe-kidney). Delivery methods to the kidney may include those in Yuan et al. (Am J Physiol Renal Physiol 295: F605-F617, 2008). The method of Yuang et al. may be applied to the engineered therapeutic polynucleotides of the present invention, which contemplates a 1-2 g subcutaneous injection of a CRISPR Cas conjugated with cholesterol to a human for delivery to the kidneys. In an embodiment, the method of Molitoris et al. (J Am Soc Nephrol 20:1754-1764, 2009) can be adapted to the engineered therapeutic polynucleotides of the present invention of the present invention and a cumulative dose of 12-20 mg/kg to a human can be used for delivery to the proximal tubule cells of the kidneys. In an embodiment, the methods of Thompson et al. (Nucleic Acid Therapeutics, Volume 22, Number 4, 2012) can be adapted to the engineered therapeutic polynucleotides of the present invention and a dose of up to 25 mg/kg can be delivered via i.v. administration. In an embodiment, the method of Shimizu et al. (J Am Soc Nephrol 21:622-633, 2010) can be adapted to the engineered therapeutic polynucleotides of the present invention and a dose of about of 10-20 μmol CRISPR Cas complexed with nanocarriers in about 1-2 liters of a physiologic fluid for i.p. administration can be used.

Other various delivery vehicles can be used to deliver the engineered therapeutic polynucleotides of the present invention to the kidney such as viral, hydrodynamic, lipid, polymer nanoparticles, aptamers and various combinations thereof (see e.g. Larson et al., Surgery, (August 2007), Vol. 142, No. 2, pp. (262-269); Hamar et al., Proc Natl Acad Sci, (October 2004), Vol. 101, No. 41, pp. (14883-14888); Zheng et al., Am J Pathol, (October 2008), Vol. 173, No. 4, pp. (973-980); Feng et al., Transplantation, (May 2009), Vol. 87, No. 9, pp. (1283-1289); Q. Zhang et al., PloS ONE, (July 2010), Vol. 5, No. 7, e11709, pp. (1-13); Kushibikia et al., J Controlled Release, (July 2005), Vol. 105, No. 3, pp. (318-331); Wang et al., Gene Therapy, (July 2006), Vol. 13, No. 14, pp. (1097-1103); Kobayashi et al., Journal of Pharmacology and Experimental Therapeutics, (February 2004), Vol. 308, No. 2, pp. (688-693); Wolfrum et al., Nature Biotechnology, (September 2007), Vol. 25, No. 10, pp. (1149-1157); Molitoris et al., J Am Soc Nephrol, (August 2009), Vol. 20, No. 8 pp. (1754-1764); Mikhaylova et al., Cancer Gene Therapy, (March 2011), Vol. 16, No. 3, pp. (217-226); Y. Zhang et al., J Am Soc Nephrol, (April 2006), Vol. 17, No. 4, pp. (1090-1101); Singhal et al., Cancer Res, (May 2009), Vol. 69, No. 10, pp. (4244-4251); Malek et al., Toxicology and Applied Pharmacology, (April 2009), Vol. 236, No. 1, pp. (97-108); Shimizu et al., J Am Soc Nephrology, (April 2010), Vol. 21, No. 4, pp. (622-633); Jiang et al., Molecular Pharmaceutics, (May-June 2009), Vol. 6, No. 3, pp. (727-737); Cao et al, J Controlled Release, (June 2010), Vol. 144, No. 2, pp. (203-212); Ninichuk et al., Am J Pathol, (March 2008), Vol. 172, No. 3, pp. (628-637); Purschke et al., Proc Natl Acad Sci, (March 2006), Vol. 103, No. 13, pp. (5173-5178). Others are described in greater detail elsewhere herein.

In an embodiment, delivery is to liver cells. In an embodiment, the liver cell is a hepatocyte. Delivery of engineered therapeutic polynucleotides of the present invention, such as one or more that encode CRISPR protein, such as Cas effector (e.g. Cas9 and/or Cas12) herein may be via viral vectors, especially AAV (and in particular AAV2/6) vectors. These can be administered by intravenous injection. A preferred target for the liver, whether in vitro or in vivo, is the albumin gene. This is a so-called ‘safe harbor” as albumin is expressed at very high levels and so some reduction in the production of albumin following successful gene editing is tolerated. It is also preferred as the high levels of expression seen from the albumin promoter/enhancer allows for useful levels of correct or transgene production (from the inserted donor template) to be achieved even if only a small fraction of hepatocytes are edited. See sites identified by Wechsler et al. (reported at the 57th Annual Meeting and Exposition of the American Society of Hematology—abstract available online at ash.confex.com/ash/2015/webprogram/Paper86495.html and presented on 6 December 2015) which can be adapted for use with the engineered therapeutic polynucleotides of the present invention.

Exemplary liver and kidney diseases that can be treated and/or prevented are described elsewhere herein.

### Treating Epithelial and Lung Diseases

In an embodiment, the disease treated or prevented by the engineered therapeutic polynucleotides of the present invention described herein can be a lung or epithelial disease. The engineered therapeutic polynucleotides of the present invention can be used for treating epithelial and/or lung diseases. The present invention also contemplates delivering the CRISPR-Cas system described herein, e.g., Cas (e.g. Cas9 and/or Cas12) effector systems, to one or both lungs via lung specific expression of an engineered therapeutic polynucleotides of the present invention that encodes one or more components of a genetic modifying system.

In an embodiment, as viral vector can be used to deliver the engineered therapeutic polynucleotides of the present invention thereof to the lungs. In an embodiment, the AAV is an AAV-1, AAV-2, AAV-5, AAV-6, and/or AAV-9 for delivery to the lungs. (see, e.g., Li et al., Molecular Therapy, vol. 17 no. 12, 2067-277 Dec. 2009). In an embodiment, the MOI can vary from 1×10³ to 4×10⁵ vector genomes/cell. In an embodiment, the delivery vector can be an RSV vector as in Zamora et al. (Am J Respir Crit Care Med Vol 183. pp 531-538, 2011. The method of Zamora et al. may be applied to the nucleic acid-targeting system of the present invention and an aerosolized CRISPR Cas, for example with a dosage of 0.6 mg/kg, may be contemplated for the present invention.

Subjects treated for a lung disease may for example receive pharmaceutically effective amount of aerosolized AAV vector system per lung endobronchially delivered while spontaneously breathing. As such, aerosolized delivery is preferred for AAV delivery in general. An adenovirus or an AAV particle may be used for delivery. Suitable gene constructs, each operably linked to one or more regulatory sequences, may be cloned into the delivery vector. In this instance, the following constructs are provided as examples: Cbh or EFla promoter for Cas (Cas (e.g. Cas9 and/or Cas12)), U6 or H1 promoter for guide RNA): A preferred arrangement is to use a CFTRdelta508 targeting guide, a repair template for deltaF508 mutation and a codon optimized Cas (e.g. Cas9 and/or Cas12) enzyme, with optionally one or more nuclear localization signal or sequence(s) (NLS(s)), e.g., two (2) NLSs.

### Treating Diseases of the Skin

The engineered therapeutic polynucleotides of the present invention described herein can be used for the treatment of skin diseases. The present invention also contemplates delivering a genetic modifying system (e.g., a CRISPR-Cas system or component thereof e.g., Cas (e.g. Cas9 and/or Cas12)), to the skin in a cell type specific manner via an engineered therapeutic polynucleotide of the present invention.

In an embodiment, delivery to the skin (intradermal delivery) of the engineered therapeutic polynucleotides of the present invention can be via one or more microneedles or microneedle containing device. For example, In an embodiment the device and methods of Hickerson et al. (Molecular Therapy—Nucleic Acids (2013) 2, e129) can be used and/or adapted to deliver the engineered therapeutic polynucleotides of the present invention, for example, at a dosage of up to 300 μl of 0.1 mg/ml CRISPR-Cas (e.g. Cas9 and/or Cas12) system or other therapeutic polynucleotide to the skin.

In an embodiment, the methods and techniques of Leachman et al. (Molecular Therapy, vol. 18 no. 2, 442-446 Feb. 2010) can be used and/or adapted for delivery of the engineered therapeutic polynucleotides of the present invention described herein to the skin.

In an embodiment, the methods and techniques of Zheng et al. (PNAS, Jul. 24, 2012, vol. 109, no. 30, 11975-11980) can be used and/or adapted for nanoparticle delivery of the engineered therapeutic polynucleotides of the present invention to the skin. In an embodiment, as dosage of about 25 nM applied in a single application can achieve gene knockdown in the skin.

### Treating Cancer

The engineered therapeutic polynucleotides of the present invention can be used for the treatment of cancer. The present invention also contemplates delivering the engineered therapeutic polynucleotides of the present invention, to a cancer cell. Also, as is described elsewhere herein the engineered therapeutic polynucleotides of the present invention can be used to modify an immune cell, such as a CAR or CAR T cell, which can then in turn be used to treat and/or prevent cancer. This is also described in WO2015161276, the disclosure of which is hereby incorporated by reference and described herein below.

Target genes suitable for the treatment or prophylaxis of cancer can include those set forth in Tables 5 and 6 and those identified at mitoMap.org. In an embodiment, target genes for cancer treatment and prevention can also include those described in WO2015048577 the disclosure of which is hereby incorporated by reference and can be adapted for and/or applied to the CRISPR-Cas system described herein.

### Additional Exemplary Diseases

Genetic Diseases and Diseases with a Genetic and/or Epigenetic Aspect

The engineered therapeutic polynucleotides of the present invention can be used to treat and/or prevent a genetic disease or a disease with a genetic and/or epigenetic aspect. The genes and conditions exemplified herein are not exhaustive. In an embodiment, a method of treating and/or preventing a genetic disease can include administering the engineered therapeutic polynucleotides of the present invention to a subject. In an embodiment, where the engineered therapeutic polynucleotides of the present invention are capable of modifying or replacing one or more copies of one or more genes associated with the genetic disease or a disease with a genetic and/or epigenetic aspect in one or more cells of the subject. In an embodiment, modifying one or more copies of one or more genes associated with a genetic disease or a disease with a genetic and/or epigenetic aspect in the subject can eliminate a genetic disease or a symptom thereof in the subject. In an embodiment, modifying one or more copies of one or more genes associated with a genetic disease or a disease with a genetic and/or epigenetic aspect in the subject can decrease the severity of a genetic disease or a symptom thereof in the subject. In an embodiment, the engineered therapeutic polynucleotides of the present invention can modify or replace one or more genes or polynucleotides associated with one or more diseases, including genetic diseases and/or those having a genetic aspect and/or epigenetic aspect, including but not limited to, any one or more set forth in Table 5. It will be appreciated that those diseases and associated genes listed herein are non-exhaustive and non-limiting. Further some genes play roles in the development of multiple diseases.

As described elsewhere herein the therapeutic polynucleotide can be a polynucleotide that can be delivered to a cell and, In an embodiment, be integrated into the genome of the cell. In an embodiment, the engineered therapeutic polynucleotides of the present invention can contain one or more polynucleotides that encode one or more CRISPR-Cas system or other genetic modifying system components. In an embodiment, the engineered therapeutic polynucleotides of the present invention, are expressed in the recipient cell and act to modify the genome of the recipient cell in a sequence specific manner. In an embodiment, the engineered therapeutic polynucleotides of the present invention were packaged and delivered by the engineered AAV capsid particles or other particles and/or compositions described herein can facilitate/mediate genome modification via a method that is not dependent on CRISPR-Cas. Such non-CRISPR-Cas genome modification systems will instantly be appreciated by those of ordinary skill in the art and are also, at least in part, described elsewhere herein. In an embodiment, modification is at a specific target sequence. In other embodiments, modification is at locations that appear to be random throughout the genome.

Examples of disease-associated genes and polynucleotides and disease specific information is available from McKusick-Nathans Institute of Genetic Medicine, Johns Hopkins University (Baltimore, Md.) and National Center for Biotechnology Information, National Library of Medicine (Bethesda, Md.), available on the World Wide Web. Any of these can be appropriate to be treated by one or more of the methods described herein. In an embodiment, the disease that can be treated with the engineered therapeutic polynucleotides of the present invention is a muscle disease or disorder, neuro-muscular disease or disorder, or a cardiomyopathy. In an embodiment, the disease or disorder is selected from any one or more of the following:
(a) an auto immune disease;
(b) a cancer;
(c) a muscular dystrophy;
(d) a neuro-muscular disease;
(e) a sugar or glycogen storage disease;
(f) an expanded repeat disease;
(g) a dominant negative disease;
(h) a cardiomyopathy;
(i) a viral disease;
(j) a progeroid disease; or
(k) any combination thereof.

In an embodiment, the expanded repeat disease is Huntington's disease, a Myotonic Dystrophy, or Facioscapulohumeral muscular dystrophy (FSHD). In an embodiment, the muscular dystrophy is Duchene muscular dystrophy, Becker Muscular dystrophy, a Limb-Girdle muscular dystrophy, an Emery Dreifuss muscular dystrophy, a myotonic dystrophy, or FSHD. In an embodiment, the myotonic dystrophy is Type 1 or Type 2. In an embodiment, the cardiomyopathy is dilated cardiomyopathy, hypertrophic cardiomyopathy, DMD-associated cardiomyopathy, or Dannon disease. In an embodiment, the sugar or glycogen storage disease is a MPS type III disease or Pompe disease. In an embodiment, the MPS type III disease, is MPS Type IIIA, IIIB, IIIC, or IIID. In an embodiment, the neuro-muscular disease is Charcot-Marie-Tooth disease or Friedreich's Ataxia.

More specifically, mutations in these genes and pathways can result in production of improper proteins or proteins in improper amounts which affect function. Such diseases can be treated with the engineered therapeutic polynucleotides of the present invention. Further examples of genes, diseases and proteins are hereby incorporated by reference from U.S. Provisional application 61/736,527 filed Dec. 12, 2012. Such genes, proteins and pathways may be the target polynucleotide of a CRISPR complex or other method of gene modification of the present invention. Examples of disease-associated and/or cell function-associated genes and polynucleotides are listed in Tables 5 and 6 Additional examples are discussed elsewhere herein.

TABLE 5
Exemplary Genetic and Other Diseases and Associated Genes

| | Primary | Additional | |
|---|---|---|---|
| | Tissues or | Tissues/ | |
| | System | Systems | |
| Disease Name | Affected | Affected | Genes |
| Achondroplasia | Bone and | | fibroblast growth factor receptor 3 |
| | Muscle | | (FGFR3) |
| Achromatopsia | eye | | CNGA3, CNGB3, GNAT2, PDE6C, |
| | | | PDE6H, ACHM2, ACHM3, |
| Acute Renal Injury | kidney | | NFkappaB, AATF, p85alpha, FAS, |
| | | | Apoptosis cascade elements (e.g. |
| | | | FASR, Caspase 2, 3, 4, 6, 7, 8, 9, 10, |
| | | | AKT, TNF alpha, IGF1, IGF1R, |
| | | | RIPK1), p53 |
| Age Related Macular | eye | | Abcr; CCL2; CC2; CP |
| Degeneration | | | (ceruloplasmin); Timp3; cathepsinD; |
| | | | VLDLR, CCR2 |
| AIDS | Immune System | | KIR3DL1, NKAT3, NKB1, AMB11, |
| | | | KIR3DS1, IFNG, CXCL12, SDF1 |
| Albinism (including | Skin, hair, eyes, | | TYR, OCA2, TYRP1, and SLC45A2, |
| oculocutaneous albinism (types | | | SLC24A5 and C10orf11 |
| 1-7) and ocular albinism) | | | |
| Alkaptonuria | Metabolism of | Tissues/organs | HGD |
| | amino acids | where homogentisic | |
| | | acid accumulates, | |
| | | particularly | |
| | | cartilage (joints), | |
| | | heart valves, | |
| | | kidneys | |
| alpha-1 antitrypsin | Lung | Liver, skin, | SERPINA1, those set forth in |
| deficiency | | vascular system, | WO2017165862, PiZ allele |
| (AATD or A1AD) | | kidneys, GI | |
| ALS | CNS | | SOD1; ALS2; ALS3; ALS5; |
| | | | ALS7; STEX; FUS; TARDBP; VEGF |
| | | | (VEGF-a; |
| | | | VEGF-b; VEGF-c); DPP6; NEFH, |
| | | | PTGS1, SLC1A2, TNFRSF10B, |
| | | | PRPH, HSP90AA1, CRIA2, IFNG, |
| | | | AMPA2 S100B, FGF2, AOX1, CS, |
| | | | TXN, RAPHJ1, MAP3K5, NBEAL1, |
| | | | GPX1, ICA1L, RAC1, MAPT, ITPR2, |
| | | | ALS2CR4, GLS, ALS2CR8, CNTFR, |
| | | | ALS2CR11, FOLH1, FAM117B, |
| | | | P4HB, CNTF, SQSTM1, STRADB, |
| | | | NAIP, NLR, YWHAQ, SLC33A1, |
| | | | TRAK2, SCA1, NIF3L1, NIF3, |
| | | | PARD3B, COX8A, CDK15, HECW1, |
| | | | HECT, C2, WW 15, NOS1, MET, |
| | | | SOD2, HSPB1, NEFL, CTSB, ANG, |
| | | | HSPA8, RNase A, VAPB, VAMP, |
| | | | SNCA, alpha HGF, CAT, ACTB, |
| | | | NEFM, TH, BCL2, FAS, CASP3, |
| | | | CLU, SMN1, G6PD, BAX, HSF1, |
| | | | RNF19A, JUN, ALS2CR12, HSPA5, |
| | | | MAPK14, APEX1, TXNRD1, NOS2, |
| | | | TIMP1, CASP9, XIAP, GLG1, EPO, |
| | | | VEGFA, ELN, GDNF, NFE2L2, |
| | | | SLC6A3, HSPA4, APOE, PSMB8, |
| | | | DCTN2, TIMP3, KIFAP3, SLC1A1, |
| | | | SMN2, CCNC, STUB1, ALS2, |
| | | | PRDX6, SYP, CABIN1, CASP1, |
| | | | GART, CDK5, ATXN3, RTN4, |
| | | | C1QB, VEGFC, HTT, PARK7, XDH, |
| | | | GFAP, MAP2, CYCS, FCGR3B, CCS, |
| | | | UBL5, MMP9m SLC18A3, TRPM7, |
| | | | HSPB2, AKT1, DEERL1, CCL2, |
| | | | NGRN, GSR, TPPP3, APAF1, |
| | | | BTBD10, GLUD1, CXCR4, S:C1A3, |
| | | | FLT1, PON1, AR, LIF, ERBB3, |
| | | | :GA:S1, CD44, TP53, TLR3, GRIA1, |
| | | | GAPDH, AMPA, GRIK1, DES, |
| | | | CHAT, FLT4, CHMP2B, BAG1, |
| | | | CHRNA4, GSS, BAK1, KDR, GSTP1, |
| | | | OGG1, IL6 |
| Alzheimer's Disease | Brain | | E1; CHIP; UCH; UBB; Tau; LRP; |
| | | | PICALM; CLU; PS1; |
| | | | SORL1; CR1; VLDLR; UBA1; |
| | | | UBA3; CHIP28; AQP1; UCHL1; |
| | | | UCHL3; APP, AAA, CVAP, AD1, |
| | | | APOE, AD2, DCP1, ACE1, MPO, |
| | | | PACIP1, PAXIP1L, PTIP, A2M, |
| | | | BLMH, BMH, PSEN1, AD3, ALAS2, |
| | | | ABCA1, BIN1, BDNF, BTNL8, |
| | | | C1ORF49, CDH4, CHRNB2, |
| | | | CKLFSF2, CLEC4E, CR1L, CSF3R, |
| | | | CST3, CYP2C, DAPK1, ESR1, |
| | | | FCAR, FCGR3B, FFA2, FGA, GAB2, |
| | | | GALP, GAPDHS, GMPB, HP, HTR7, |
| | | | IDE, IF127, IFI6, IFIT2, IL1RN, IL- |
| | | | 1RA, IL8RA, IL8RB, JAG1, KCNJ15, |
| | | | LRP6, MAPT, MARK4, MPHOSPH1, |
| | | | MTHFR, NBN, NCSTN, NIACR2, |
| | | | NMNAT3, NTM, ORM1, P2RY13, |
| | | | PBEF1, PCK1, PICALM, PLAU, |
| | | | PLXNC1, PRNP, PSEN1, PSEN2, |
| | | | PTPRA, RALGPS2, RGSL2, |
| | | | SELENBP1, SLC25A37, SORL1, |
| | | | Mitoferrin-1, TF, TFAM, TNF, |
| | | | TNFRSF10C, UBE1C |
| Amyloidosis | | | APOA1, APP, AAA, CVAP, AD1, |
| | | | GSN, FGA, LYZ, TTR, PALB |
| Amyloid neuropathy | | | TTR, PALB |
| Anemia | Blood | | CDAN1, CDA1, RPS19, DBA, PKLR, |
| | | | PK1, NT5C3, UMPH1, PSN1, RHAG, |
| | | | RH50A, NRAMP2, SPTB, ALAS2, |
| | | | ANH1, ASB, ABCB7, ABC7, ASAT |
| Angelman Syndrome | Nervous system, | | UBE3A |
| | brain | | |
| Attention Deficit Hyperactivity | Brain | | PTCHD1 |
| Disorder (ADHD) | | | |
| Autoimmune lymphoproliferative | Immune system | | TNFRSF6, APT1, FAS, CD95, |
| syndrome | | | ALPS1A |
| Autism, Autism spectrum | Brain | | PTCHD1; Mecp2; BZRAP1; MDGA2; |
| disorders (ASDs), including | | | Sema5A; Neurexin 1; GLO1, RTT, |
| Asperger's and a general | | | PPMX, MRX16, RX79, NLGN3, |
| diagnostic category called | | | NLGN4, KIAA1260, AUTSX2, |
| Pervasive Developmental | | | FMR1, FMR2; FXR1; FXR2; |
| Disorders (PDDs) | | | MGLUR5, ATP10C, CDH10, GRM6, |
| | | | MGLUR6, CDH9, CNTN4, NLGN2, |
| | | | CNTNAP2, SEMA5A, DHCR7, |
| | | | NLGN4X, NLGN4Y, DPP6, NLGN5, |
| | | | EN2, NRCAM, MDGA2, NRXN1, |
| | | | FMR2, AFF2, FOXP2, OR4M2, |
| | | | OXTR, FXR1, FXR2, PAH, |
| | | | GABRA1, PTEN, GABRA5, PTPRZ1, |
| | | | GABRB3, GABRG1, HIRIP3, |
| | | | SEZ6L2, HOXA1, SHANK3, IL6, |
| | | | SHBZRAP1, LAMB1, SLC6A4, |
| | | | SERT, MAPK3, TAS2R1, MAZ, |
| | | | TSC1, MDGA2, TSC2, MECP2, |
| | | | UBE3A, WNT2, see also |
| | | | 20110023145 |
| autosomal dominant polycystic | kidney | liver | PKD1, PKD2 |
| kidney disease (ADPKD) - | | | |
| (includes diseases such as von | | | |
| Hippel-Lindau disease and | | | |
| tubreous sclerosis complex | | | |
| disease) | | | |
| Autosomal Recessive Polycystic | kidney | liver | PKDH1 |
| Kidney Disease (ARPKD) | | | |
| Ataxia-Telangiectasia (a.k.a | Nervous system, | various | ATM |
| Louis Bar syndrome) | immune system | | |
| B-Cell Non-Hodgkin Lymphoma | | | BCL7A, BCL7 |
| Bardet-Biedl syndrome | Eye, | Liver, ear, | ARL6, BBS1, BBS2, BBS4, BBS5, |
| | musculoskeletal | gastrointestinal | BBS7, BBS9, BBS10, BBS12, |
| | system, kidney, | system, brain | CEP290, INPP5E, LZTFL1, MKKS, |
| | reproductive | | MKS1, SDCCAG8, TRIM32, TTC8 |
| | organs | | |
| Bare Lymphocyte Syndrome | blood | | TAPBP, TPSN, TAP2, ABCB3, PSF2, |
| | | | RING11, MHC2TA, C2TA, RFX5, |
| | | | RFXAP, RFX5 |
| Bartter's Syndrome (types I, II, | kidney | | SLC12A1 (type I), KCNJ1 (type II), |
| III, IVA and B, and V) | | | CLCNKB (type III), BSND (type IV |
| | | | A), or both the CLCNKA CLCNKB |
| | | | genes (type IV B), CASR (type V). |
| Becker muscular dystrophy | Muscle | | DMD, BMD, MYF6 |
| Best Disease (Vitelliform | eye | | VMD2 |
| Macular Dystrophy type 2 ) | | | |
| Bleeding Disorders | blood | | TBXA2R, P2RX1, P2X1 |
| Blue Cone Monochromacy | eye | | OPN1LW, OPN1MW, and LCR |
| Breast Cancer | Breast tissue | | BRCA1, BRCA2, COX-2 |
| Bruton's Disease (aka X-linked | Immune system, | | BTK |
| Agammglobulinemia) | specifically B | | |
| | cells | | |
| Cancers (e.g., lymphoma, chronic | Various | | FAS, BID, CTLA4, PDCD1, CBLB, |
| lymphocytic leukemia (CLL), B | | | PTPN6, TRAC, TRBC, those |
| cell acute lymphocytic leukemia | | | described in WO2015048577 |
| (B-ALL), acute lymphoblastic | | | |
| leukemia, acute myeloid | | | |
| leukemia, non-Hodgkin's | | | |
| lymphoma (NHL), diffuse large | | | |
| cell lymphoma (DLCL), multiple | | | |
| myeloma, renal cell carcinoma | | | |
| (RCC), neuroblastoma, colorectal | | | |
| cancer, breast cancer, ovarian | | | |
| cancer, melanoma, sarcoma, | | | |
| prostate cancer, lung cancer, | | | |
| esophageal cancer, hepatocellular | | | |
| carcinoma, pancreatic cancer, | | | |
| astrocytoma, mesothelioma, head | | | |
| and neck cancer, and | | | |
| medulloblastoma | | | |
| Cardiovascular Diseases | heart | Vascular system | IL1B, XDH, TP53, PTGS, MB, IL4, |
| | | | ANGPT1, ABCGu8, CTSK, PTGIR, |
| | | | KCNJ11, INS, CRP, PDGFRB, |
| | | | CCNA2, PDGFB, KCNJ5, KCNN3, |
| | | | CAPN10, ADRA2B, ABCG5, |
| | | | PRDX2, CPAN5, PARP14, MEX3C, |
| | | | ACE, RNF, IL6, TNF, STN, |
| | | | SERPINE1, ALB, ADIPOQ, APOB, |
| | | | APOE, LEP, MTHFR, APOA1, |
| | | | EDN1, NPPB, NOS3, PPARG, PLAT, |
| | | | PTGS2, CETP, AGTR1, HMGCR, |
| | | | IGF1, SELE, REN, PPARA, PON1, |
| | | | KNG1, CCL2, LPL, VWF, F2, |
| | | | ICAM1, TGFB, NPPA, IL10, EPO, |
| | | | SOD1, VCAM1, IFNG, LPA, MPO, |
| | | | ESR1, MAPK, HP, F3, CST3, COG2, |
| | | | MMP9, SERPINC1, F8, HMOX1, |
| | | | APOC3, IL8, PROL1, CBS, NOS2, |
| | | | TLR4, SELP, ABCA1, AGT, LDLR, |
| | | | GPT, VEGFA, NR3C2, IL18, NOS1, |
| | | | NR3C1, FGB, HGF, IL1A, AKT1, |
| | | | LIPC, HSPD1, MAPK14, SPP1, |
| | | | ITGB3, CAT, UTS2, THBD, F10, CP, |
| | | | TNFRSF11B, EGFR, MMP2, PLG, |
| | | | NPY, RHOD, MAPK8, MYC, FN1, |
| | | | CMA1, PLAU, GNB3, ADRB2, |
| | | | SOD2, F5, VDR, ALOX5, HLA- |
| | | | DRB1, PARP1, CD40LG, PON2, |
| | | | AGER, IRS1, PTGS1, ECE1, F7, |
| | | | IRMN, EPHX2, IGFBP1, MAPK10, |
| | | | FAS, ABCB1, JUN, IGFBP3, CD14, |
| | | | PDE5A, AGTR2, CD40, LCAT, |
| | | | CCR5, MMP1, TIMP1, ADM, |
| | | | DYT10, STAT3, MMP3, ELN, USF1, |
| | | | CFH, HSPA4, MMP12, MME, F2R, |
| | | | SELL, CTSB, ANXA5, ADRB1, |
| | | | CYBA, FGA, GGT1, LIPG, HIF1A, |
| | | | CXCR4, PROC, SCARB1, CD79A, |
| | | | PLTP, ADD1, FGG, SAA1, KCNH2, |
| | | | DPP4, NPR1, VTN, KIAA0101, FOS, |
| | | | TLR2, PPIG, IL1R1, AR, CYP1A1, |
| | | | SERPINA1, MTR, RBP4, APOA4, |
| | | | CDKN2A, FGF2, EDNRB, ITGA2, |
| | | | VLA-2, CABIN1, SHBG, HMGB1, |
| | | | HSP90B2P, CYP3A4, GJA1, CAV1, |
| | | | ESR2, LTA, GDF15, BDNF, |
| | | | CYP2D6, NGF, SP1, TGIF1, SRC, |
| | | | EGF, PIK3CG, HLA-A, KCNQ1, |
| | | | CNR1, FBN1, CHKA, BEST1, |
| | | | CTNNB1, IL2, CD36, PRKAB1, TPO, |
| | | | ALDH7A1, CX3CR1, TH, F9, CH1, |
| | | | TF, HFE, IL17A, PTEN, GSTM1, |
| | | | DMD, GATA4, F13A1, TTR, FABP4, |
| | | | PON3, APOC1, INSR, TNFRSF1B, |
| | | | HTR2A, CSF3, CYP2C9, TXN, |
| | | | CYP11B2, PTH, CSF2, KDR, |
| | | | PLA2G2A, THBS1, GCG, RHOA, |
| | | | ALDH2, TCF7L2, NFE2L2, |
| | | | NOTCH1, UGT1A1, IFNA1, PPARD, |
| | | | SIRT1, GNHR1, PAPPA, ARR3, |
| | | | NPPC, AHSP, PTK2, IL13, MTOR, |
| | | | ITGB2, GSTT1, IL6ST, CPB2, |
| | | | CYP1A2, HNF4A, SLC64A, |
| | | | PLA2G6, TNFSF11, SLC8A1, F2RL1, |
| | | | AKR1A1, ALDH9A1, BGLAP, |
| | | | MTTP, MTRR, SULT1A3, RAGE, |
| | | | C4B, P2RY12, RNLS, CREB1, |
| | | | POMC, RAC1, LMNA, CD59, |
| | | | SCM5A, CYP1B1, MIF, MMP13, |
| | | | TIMP2, CYP19A1, CUP21A2, |
| | | | PTPN22, MYH14, MBL2, SELPLG, |
| | | | AOC3, CTSL1, PCNA, IGF2, ITGB1, |
| | | | CAST, CXCL12, IGHE, KCNE1, |
| | | | TFRC, COL1A1, COL1A2, IL2RB, |
| | | | PLA2G10, ANGPT2, PROCR, NOX4, |
| | | | HAMP, PTPN11, SLCA1, IL2RA, |
| | | | CCL5, IRF1, CF:AR, CA:CA, EIF4E, |
| | | | GSTP1, JAK2, CYP3A5, HSPG2, |
| | | | CCL3, MYD88, VIP, SOAT1, |
| | | | ADRBK1, NR4A2, MMP8, NPR2, |
| | | | GCH1, EPRS, PPARGC1A, F12, |
| | | | PECAM1, CCL4, CERPINA34, |
| | | | CASR, FABP2, TTF2, PROS1, CTF1, |
| | | | SGCB, YME1L1, CAMP, ZC3H12A, |
| | | | AKR1B1, MMP7, AHR, CSF1, |
| | | | HDAC9, CTGF, KCNMA1, UGT1A, |
| | | | PRKCA, COMT, S100B, EGR1, PRL, |
| | | | IL15, DRD4, CAMK2G, SLC22A2, |
| | | | CCL11, PGF, THPO, GP6, TACR1, |
| | | | NTS, HNF1A, SST, KCDN1, |
| | | | LOC646627, TBXAS1, CUP2J2, |
| | | | TBXA2R, ADH1C, ALOX12, AHSG, |
| | | | BHMT, GJA4, SLC25A4, ACLY, |
| | | | ALOX5AP, NUMA1, CYP27B1, |
| | | | CYSLTR2, SOD3, LTC4S, UCN, |
| | | | GHRL, APOC2, CLEC4A, |
| | | | KBTBD10, TNC, TYMS, SHC1, |
| | | | LRP1, SOCS3, ADH1B, KLK3, |
| | | | HSD11B1, VKORC1, SERPINB2, |
| | | | TNS1, RNF19A, EPOR, ITGAM, |
| | | | PITX2, MAPK7, FCGR3A, LEEPR, |
| | | | ENG, GPX1, GOT2, HRH1, NR112, |
| | | | CRH, HTR1A, VDAC1, HPSE, |
| | | | SFTPD, TAP2, RMF123, PTK2Bm |
| | | | NTRK2, IL6R, ACHE, GLP1R, GHR, |
| | | | GSR, NQO1, NR5A1, GJB2, |
| | | | SLC9A1, MAOA, PCSK9, FCGR2A, |
| | | | SERPINF1, EDN3, UCP2, TFAP2A, |
| | | | C4BPA, SERPINF2, TYMP, ALPP, |
| | | | CXCR2, SLC3A3, ABCG2, ADA, |
| | | | JAK3, HSPA1A, FASN, FGF1, F11, |
| | | | ATP7A, CR1, GFPA, ROCK1, |
| | | | MECP2, MYLK, BCHE, LIPE, |
| | | | ADORA1, WRN, CXCR3, CD81, |
| | | | SMAD7, LAMC2, MAP3K5, CHGA, |
| | | | IAPP, RHO, ENPP1, PTHLH, NRG1, |
| | | | VEGFC, ENPEP, CEBPB, NAGLU,. |
| | | | F2RL3, CX3CL1, BDKRB1, |
| | | | ADAMTS13, ELANE, ENPP2, CISH, |
| | | | GAST, MYOC, ATP1A2, NF1, GJB1, |
| | | | MEF2A, VCL, BMPR2, TUBB, |
| | | | CDC42, KRT18, HSF1, MYB, |
| | | | PRKAA2, ROCK2, TFP1, PRKG1, |
| | | | BMP2, CTNND1, CTH, CTSS, |
| | | | VAV2, NPY2R, IGFBP2, CD28, |
| | | | GSTA1, PPIA, APOH, S100A8, IL11, |
| | | | ALOX15, FBLN1, NR1H3, SCD, GIP, |
| | | | CHGB, PRKCB, SRD5A1, HSD11B2, |
| | | | CALCRL, GALNT2, ANGPTL4, |
| | | | KCNN4, PIK3C2A, HBEGF, |
| | | | CYP7A1, HLA-DRB5, BNIP3, |
| | | | GCKR, S100A12, PADI4, HSPA14, |
| | | | CXCR1, H19, KRTAP19-3, IDDM2, |
| | | | RAC2, YRY1, CLOCK, NGFR, DBH, |
| | | | CHRNA4, CACNA1C, PRKAG2, |
| | | | CHAT, PTGDS, NR1H2, TEK, |
| | | | VEGFB, MEF2C, MAPKAPK2, |
| | | | TNFRSF11A, HSPA9, CYSLTR1, |
| | | | MAT1A, OPRL1, IMPA1, CLCN2, |
| | | | DLD, PSMA6, PSMB8, CHI3L1, |
| | | | ALDH1B1, PARP2, STAR, LBP, |
| | | | ABCC6, RGS2, EFNB2, GJB6, |
| | | | APOA2, AMPD1, DYSF, |
| | | | FDFT1, EMD2, CCR6, GJB3, IL1RL1, |
| | | | ENTPD1, BBS4, CELSR2, F11R, |
| | | | RAPGEF3, HYAL1, ZNF259, |
| | | | ATOX1, ATF6, KHK, SAT1, GGH, |
| | | | TIMP4, SLC4A4, PDE2A, PDE3B, |
| | | | FADS1, FADS2, TMSB4X, TXNIP, |
| | | | LIMS1, RHOB, LY96, FOXO1, |
| | | | PNPLA2, TRH, GJC1, S:C17A5, FTO, |
| | | | GJD2, PRSC1, CASP12, GPBAR1, |
| | | | PXK, IL33, TRIB1, PBX4, NUPR1, |
| | | | 15-SEP, CILP2, TERC, GGT2, |
| | | | MTCO1, UOX, AVP, ANGPLT3 |
| Cataract | eye | | CRYAA, CRYA1, CRYBB2, CRYB2, |
| | | | PITX3, BFSP2, CP49, CP47, CRYAA, |
| | | | CRYA1, PAX6, AN2, MGDA, |
| | | | CRYBA1, CRYB1, CRYGC, CRYG3, |
| | | | CCL, LIM2, MP19, CRYGD, CRYG4, |
| | | | BFSP2, CP49, CP47, HSF4, CTM, |
| | | | HSF4, CTM, MIP, AQP0, CRYAB, |
| | | | CRYA2, CTPP2, CRYBB1, CRYGD, |
| | | | CRYG4, CRYBB2, CRYB2, CRYGC, |
| | | | CRYG3, CCL, CRYAA, CRYA1, |
| | | | GJA8, CX50, CAE1, GJA3, CX46, |
| | | | CZP3, CAE3, CCM1, CAM, KRIT1 |
| CDKL-5 Deficiencies or | Brain, CNS | | CDKL5 |
| Mediated Diseases | | | |
| Charcot-Marie-Tooth (CMT) | Nervous system | Muscles | PMP22 (CMT1A and E), MPZ |
| disease (Types 1, 2, 3, 4,) | | (dystrophy) | (CMT1B), LITAF (CMT1C), EGR2 |
| | | | (CMT1D), NEFL (CMT1F), GJB1 |
| | | | (CMT1X), MFN2 (CMT2A), KIF1B |
| | | | (CMT2A2B), RAB7A (CMT2B), |
| | | | TRPV4 (CMT2C), GARS (CMT2D), |
| | | | NEFL (CMT2E), GAPD1 (CMT2K), |
| | | | HSPB8 (CMT2L), DYNC1H1, |
| | | | CMT2O), LRSAM1 (CMT2P), |
| | | | IGHMBP2 (CMT2S), MORC2 |
| | | | (CMT2Z), GDAP1 (CMT4A), |
| | | | MTMR2 or SBF2/MTMR13 |
| | | | (CMT4B), SH3TC2 (CMT4C), |
| | | | NDRG1 (CMT4D), PRX (CMT4F), |
| | | | FIG. 4 (CMT4J), NT-3 |
| Chediak-Higashi Syndrome | Immune system | Skin, hair, eyes, | LYST |
| | | neurons | |
| Choroidermia | | | CHM, REP1, |
| Chorioretinal atrophy | eye | | PRDM13, RGR, TEAD1 |
| Chronic Granulomatous Disease | Immune system | | CYBA, CYBB, NCF1, NCF2, NCF4 |
| Chronic Mucocutaneous | Immune system | | AIRE, CARD9, CLEC7A IL12B, |
| Candidiasis | | | IL12B1, IL1F, IL17RA, IL17RC, |
| | | | RORC, STAT1, STAT3, TRAF31P2 |
| Cirrhosis | liver | | KRT18, KRT8, CIRH1A, NAIC, |
| | | | TEX292, KIAA1988 |
| HNPCC: | | | |
| Colon cancer (Familial | Gastrointestinal | | FAP: APC HNPCC: |
| adenomatous polyposis (FAP) | | | MSH2, MLH1, PMS2, SH6, PMS1 |
| and hereditary nonpolyposis | | | |
| colon cancer (HNPCC)) | | | |
| Combined Immunodeficiency | Immune System | | IL2RG, SCIDX1, SCIDX, IMD4); |
| | | | HIV-1 (CCL5, SCYA5, D17S136E, |
| | | | TCP228 |
| Cone(-rod) dystrophy | eye | | AIPL1, CRX, GUA1A, GUCY2D, |
| | | | PITPM3, PROM1, PRPH2, RIMS1, |
| | | | SEMA4A, ABCA4, ADAM9, ATF6, |
| | | | C21ORF2, C8ORF37, CACNA2D4, |
| | | | CDHR1, CERKL, CNGA3, CNGB3, |
| | | | CNNM4, CNAT2, IFT81, KCNV2, |
| | | | PDE6C, PDE6H, POC1B, RAX2, |
| | | | RDH5, RPGRIP1, TTLL5, RetCG1, |
| | | | GUCY2E |
| Congenital Stationary Night | eye | | CABP4, CACNA1F, CACNA2D4, |
| Blindness | | | GNAT1, CPR179, GRK1, GRM6, |
| | | | LRIT3, NYX, PDE6B, RDH5, RHO, |
| | | | RLBP1, RPE65, SAG, SLC24A1, |
| | | | TRPM1, |
| Congenital Fructose Intolerance | Metabolism | | ALDOB |
| Cori's Disease (Glycogen Storage | Various- | | AGL |
| Disease Type III) | wherever | | |
| | glycogen | | |
| | accumulates, | | |
| | particularly | | |
| | liver, heart, | | |
| | skeletal muscle | | |
| Corneal clouding and dystrophy | eye | | APOA1, TGFBI, CSD2, CDGG1, |
| | | | CSD, BIGH3, CDG2, TACSTD2, |
| | | | TROP2, M1S1, VSX1, RINX, PPCD, |
| | | | PPD, KTCN, COL8A2, FECD, |
| | | | PPCD2, PIP5K3, CFD |
| Cornea plana congenital | | | KERA, CNA2 |
| Cri du chat Syndrome, also | | | Deletions involving only band 5p15.2 |
| known as 5p syndrome and cat | | | to the entire short arm of chromosome |
| cry syndrome | | | 5, e.g. CTNND2, TERT, |
| Cystic Fibrosis (CF) | Lungs and | Pancreas, liver, | CTFR, ABCC7, CF, MRP7, SCNN1A, |
| | respiratory | digestive | those described in WO2015157070 |
| | system | system, | |
| | | reproductive | |
| | | system, | |
| | | exocrine, glands, | |
| Diabetic nephropathy | kidney | | Gremlin, 12/15- lipoxygenase, TIM44, |
| Dent Disease (Types 1 and 2) | Kidney | | Type 1: CLCN5, Type 2: ORCL |
| Dentatorubro-Pallidoluysian | CNS, brain, | | Atrophin-1 and Atn1 |
| Atrophy (DRPLA) (aka Haw | muscle | | |
| River and Naito-Oyanagi | | | |
| Disease) | | | |
| Down Syndrome | various | | Chromosome 21 trisomy |
| Drug Addiction | Brain | | Prkce; Drd2; Drd4; ABAT; |
| | | | GRIA2; Grm5; Grin1; Htr1b; Grin2a; |
| | | | Drd3; Pdyn; Gria1 |
| Duane syndrome (Types 1, 2, and | eye | | CHN1, indels on chromosomes 4 and 8 |
| 3, including subgroups A, B and | | | |
| C). Other names for this | | | |
| condition include: Duane's | | | |
| Retraction Syndrome (or DR | | | |
| syndrome), Eye Retraction | | | |
| Syndrome, Retraction Syndrome, | | | |
| Congenital retraction syndrome | | | |
| and Stilling-Turk-Duane | | | |
| Syndrome | | | |
| Duchenne muscular dystrophy | muscle | Cardiovascular, | DMD, BMD, dystrophin gene, intron |
| (DMD) | | respiratory | flanking exon 51 of DMD gene, exon |
| | | | 51 mutations in DMD gene, see also |
| | | | WO2013163628 and US Pat. Pub. |
| | | | 20130145487 |
| Edward's Syndrome | | | Complete or partial trisomy of |
| (Trisomy 18) | | | chromosome 18 |
| Ehlers-Danlos Syndrome (Types | Various | | COL5A1, COL5A2, COL1A1, |
| I-VI) | depending on | | COL3A1, TNXB, PLOD1, COL1A2, |
| | type: including | | FKBP14 and ADAMTS2 |
| | musculoskeletal, | | |
| | eye, vasculature, | | |
| | immune, and | | |
| | skin | | |
| Emery-Dreifuss muscular | muscle | | LMNA, LMN1, EMD2, FPLD, |
| dystrophy | | | CMD1A, HGPS, LGMD1B, LMNA, |
| | | | LMN1, EMD2, FPLD, CMD1A |
| Enhanced S-Cone Syndrome | eye | | NR2E3, NRL |
| Fabry's Disease | Various - | | GLA |
| | including skin, | | |
| | eyes, and | | |
| | gastrointestinal | | |
| | system, kidney, | | |
| | heart, brain, | | |
| | nervous system | | |
| Facioscapulohumeral muscular | muscles | | FSHMD1A, FSHD1A, FRG1, |
| dystrophy | | | |
| Factor H and Factor H-like 1 | blood | | HF1, CFH, HUS |
| Factor V Leiden thrombophilia | blood | | Factor V (F5) |
| and Factor V deficiency | | | |
| Factor V and Factor VII | blood | | MCFD2 |
| deficiency | | | |
| Factor VII deficiency | blood | | F7 |
| Factor X deficiency | blood | | F10 |
| Factor XI deficiency | blood | | F11 |
| Factor XII deficiency | blood | | F12, HAF |
| Factor XIIIA deficiency | blood | | F13A1, F13A |
| Factor XIIIB deficiency | blood | | F13B |
| Familial Hypercholestereolemia | Cardiovascular | | APOB, LDLR, PCSK9 |
| | system | | |
| Familial Mediterranean Fever | Various- | Heart, kidney, | MEFV |
| (FMF) also called recurrent | organs/tissues | brain/CNS, | |
| polyserositis or familial | with serous or | reproductive | |
| paroxysmal polyserositis | synovial | organs | |
| | membranes, | | |
| | skin, joints | | |
| Fanconi Anemia | Various - blood | | FANCA, FACA, FA1, FA, FAA, |
| | (anemia), | | FAAP95, FAAP90, FLJ34064, |
| | immune system, | | FANCC, FANCG, RAD51, BRCA1, |
| | cognitive, | | BRCA2, BRIP1, BACH1, FANCJ, |
| | kidneys, eyes, | | FANCB, FANCD1, FANCD2, |
| | musculoskeletal | | FANCD, FAD, FANCE, FACE, |
| | | | FANCF, FANCI, ERCC4, FANCL, |
| | | | FANCM, PALB2, RAD51C, SLX4, |
| | | | UBE2T, FANCB, XRCC9, PHF9, |
| | | | KIAA1596 |
| Fanconi Syndrome Types I | kidneys | | FRTS1, GATM |
| (Childhood onset) and II (Adult | | | |
| Onset) | | | |
| Fragile X syndrome and related | brain | | FMR1, FMR2; FXR1; FXR2; |
| disorders | | | mGLUR5 |
| Fragile XE Mental Retardation | Brain, nervous | | FMR1 |
| (aka Martin Bell syndrome) | system | | |
| Friedreich Ataxia (FRDA) | Brain, nervous | heart | FXN/X25 |
| | system | | |
| Fuchs endothelial corneal | Eye | | TCF4; COL8A2 |
| dystrophy | | | |
| Galactosemia | Carbohydrate | Various-where | GALT, GALK1, and GALE |
| | metabolism | galactose | |
| | disorder | accumulates - | |
| | | liver, brain, eyes | |
| Gastrointestinal Epithelial | | | CISH |
| Cancer, GI cancer | | | |
| Gaucher Disease (Types 1, 2, and | Fat metabolism | Various-liver, | GBA |
| 3, as well as other unusual forms | disorder | spleen, blood, | |
| that may not fit into these types) | | CNS, skeletal | |
| | | system | |
| Griscelli syndrome | | | |
| Glaucoma | eye | | MYOC, TIGR, GLC1A, JOAG, |
| | | | GPOA, OPTN, GLC1E, FIP2, HYPL, |
| | | | NRP, CYP1B1, GLC3A, OPA1, NTG, |
| | | | NPG, CYP1B1, GLC3A, those |
| | | | described in WO2015153780 |
| Glomerulo sclerosis | kidney | | CC chemokine ligand 2 |
| Glycogen Storage Diseases | Metabolism | | SLC2A2, GLUT2, G6PC, G6PT, |
| Types I-VI -See also Cori's | Diseases | | G6PT1, GAA, LAMP2, LAMPB, |
| Disease, Pompe's Disease, | | | AGL, GDE, GBE1, GYS2, PYGL, |
| McArdle's disease, Hers Disease, | | | PFKM, see also Cori's Disease, |
| and Von Gierke's disease | | | Pompe's Disease, McArdle's disease, |
| | | | Hers Disease, and Von Gierke's |
| | | | disease |
| RBC Glycolytic enzyme | blood | | any mutations in a gene for an enzyme |
| deficiency | | | in the glycolysis pathway including |
| | | | mutations in genes for hexokinases I |
| | | | and II, glucokinase, phosphoglucose |
| | | | isomerase, phosphofructokinase, |
| | | | aldolase Bm triosephosphate |
| | | | isomerease, glyceraldehydee-3- |
| | | | phosphate dehydrogenase, |
| | | | phosphoglycerokinase, |
| | | | phosphoglycerate mutase, enolase I, |
| | | | pyruvate kinase |
| Hartnup's disease | Malabsorption | Various- brain, | SLC6A19 |
| | disease | gastrointestinal, | |
| | | skin, | |
| Hearing Loss | ear | | NOX3, Hes5, BDNF, |
| Hemochromatosis (HH) | Iron absorption | Various- | HFE and H63D |
| | regulation | wherever iron | |
| | disease | accumulates, | |
| | | liver, heart, | |
| | | pancreas, joints, | |
| | | pituitary gland | |
| Hemophagocytic | blood | | PRF1, HPLH2, UNC13D, MUNC13- |
| lymphohistiocytosis disorders | | | 4, HPLH3, HLH3, FHL3 |
| Hemorrhagic disorders | blood | | PI, ATT, F5 |
| Hers disease (Glycogen storage | liver | muscle | PYGL |
| disease Type VI) | | | |
| Hereditary angioedema (HAE) | | | kalikrein B1 |
| Hereditary Hemorrhagic | Skin and | | ACVRL1, ENG and SMAD4 |
| Telangiectasia (Osler-Weber- | mucous | | |
| Rendu Syndrome) | membranes | | |
| Hereditary Spherocytosis | blood | | NK1, EPB42, SLC4A1, SPTA1, and |
| | | | SPTB |
| Hereditary Persistence of Fetal | blood | | HBG1, HBG2, BCL11A, promoter |
| Hemoglobin | | | region of HBG 1 and/or 2 (in the |
| | | | CCAAT box) |
| Hemophilia (hemophilia A | blood | | A: FVIII, F8C, HEMA |
| (Classic) a B (aka Christmas | | | B: FVIX, HEMB, FIX |
| disease) and C) | | | C: F9, F11 |
| Hepatic adenoma | liver | | TCF1, HNF1A, MODY3 |
| Hepatic failure, early onset, and | liver | | SCOD1, SCO1 |
| neurologic disorder | | | |
| Hepatic lipase deficiency | liver | | LIPC |
| Hepatoblastoma, cancer and | liver | | CTNNB1, PDGFRL, PDGRL, PRLTS, |
| carcinomas | | | AXIN1, AXIN, CTNNB1, TP53, P53, |
| | | | LFS1, IGF2R, MPRI, MET, CASP8, |
| | | | MCH5 |
| Hermansky-Pudlak syndrome | Skin, eyes, | | HPS1, HPS3, HPS4, HPS5, HPS6, |
| | blood, lung, | | HPS7, DTNBP1, BLOC1, BLOC1S2, |
| | kidneys, | | BLOC3 |
| | intestine | | |
| HIV susceptibility or infection | Immune system | | IL10, CSIF, CMKBR2, CCR2, |
| | | | CMKBR5, CCCKR5 (CCR5), those in |
| | | | WO2015148670A1 |
| Holoprosencephaly (HPE) | brain | | ACVRL1, ENG, SMAD4 |
| (Alobar, Semilobar, and Lobar) | | | |
| Homocystinuria | Metabolic | Various- | CBS, MTHFR, MTR, MTRR, and |
| | disease | connective | MMADHC |
| | | tissue, muscles, | |
| | | CNS, | |
| | | cardiovascular | |
| | | system | |
| HPV | | | HPV16 and HPV18 E6/E7 |
| HSV1, HSV2, and related | eye | | HSV1 genes (immediate early and late |
| keratitis | | | HSV-1 genes (UL1, 1.5, 5, 6, 8, 9, 12, |
| | | | 15, 16, 18, 19, 22, 23, 26, 26.5, 27, 28, |
| | | | 29, 30, 31, 32, 33, 34, 35, 36, 37, 38, |
| | | | 42, 48, 49.5, 50, 52, 54, S6, RL2, RS1, |
| | | | those described in WO2015153789, |
| | | | WO2015153791 |
| Hunter's Syndrome (aka | Lysosomal | Various- liver, | IDS |
| Mucopolysaccharidosis type II) | storage disease | spleen, eye, | |
| | | joint, heart, | |
| | | brain, skeletal | |
| Huntington's disease (HD) and | Brain, nervous | | HD, HTT, IT15, PRNP, PRIP, JPH3, |
| HD-like disorders | system | | JP3, HDL2, TBP, SCA17, PRKCE; |
| | | | IGF1; EP300; RCOR1; PRKCZ; |
| | | | HDAC4; and TGM2, and those |
| | | | described in WO2013130824, |
| | | | WO2015089354 |
| Hurler's Syndrome (aka | Lysosomal | Various- liver, | IDUA, α-L-iduronidase |
| mucopolysaccharidosis type I H, | storage disease | spleen, eye, | |
| MPS IH) | | joint, heart, | |
| | | brain, skeletal | |
| Hurler-Scheie syndrome (aka | Lysosomal | Various- liver, | IDUA, α-L-iduronidase |
| mucopolysaccharidosis type I H- | storage disease | spleen, eye, | |
| S, MPS I H-S) | | joint, heart, | |
| | | brain, skeletal | |
| hyaluronidase deficiency (aka | Soft and | | HYAL1 |
| MPS IX) | connective | | |
| | tissues | | |
| Hyper IgM syndrome | Immune system | | CD40L |
| Hyper- tension caused renal | kidney | | Mineral corticoid receptor |
| damage | | | |
| Immunodeficiencies | Immune System | | CD3E, CD3G, AICDA, AID, HIGM2, |
| | | | TNFRSF5, CD40, UNG, DGU, |
| | | | HIGM4, TNFSF5, CD40LG, HIGM1, |
| | | | IGM, FOXP3, IPEX, AIID, XPID, |
| | | | PIDX, TNFRSF14B, TACI |
| Inborn errors of metabolism: | Metabolism | Various organs | See also: Carbohydrate metabolism |
| including urea cycle disorders, | diseases, liver | and cells | disorders (e.g. galactosemia), Amino |
| organic acidemias), fatty acid | | | acid Metabolism disorders (e.g. |
| oxidation defects, amino | | | phenylketonuria), Fatty acid |
| acidopathies, carbohydrate | | | metabolism (e.g. MCAD deficiency), |
| disorders, mitochondrial | | | Urea Cycle disorders (e.g. |
| disorders | | | Citrullinemia), Organic acidemias (e.g. |
| | | | Maple Syrup Urine disease), |
| | | | Mitochondrial disorders (e.g. |
| | | | MELAS), peroxisomal disorders (e.g. |
| | | | Zellweger syndrome) |
| Inflammation | Various | | IL-10; IL-1 (IL-1a; IL-1b); IL-13; IL- |
| | | | 17 (IL-17a (CTLA8); IL- |
| | | | 17b; IL-17c; IL-17d; IL-17f); II-23; |
| | | | Cx3cr1; ptpn22; TNFa; |
| | | | NOD2/CARD15 for IBD; IL-6; IL-12 |
| | | | (IL-12a; IL-12b); |
| | | | CTLA4; Cx3cl1 |
| Inflammatory Bowel Diseases | Gastrointestinal | Joints, skin | NOD2, IRGM, LRRK2, ATG5, |
| (e.g. Ulcerative Colitis and | | | ATG16L1, IRGM, GATM, ECM1, |
| Chron's Disease) | | | CDH1, LAMB1, HNF4A, GNA12, |
| | | | IL10, CARD9/15. CCR6, IL2RA, |
| | | | MST1, TNFSF15, REL, STAT3, |
| | | | IL23R, IL12B, FUT2 |
| Interstitial renal fibrosis | kidney | | TGF-β type II receptor |
| Job's Syndrome (aka Hyper IgE | Immune System | | STAT3, DOCK8 |
| Syndrome) | | | |
| Juvenile Retinoschisis | eye | | RS1, XLRS1 |
| Kabuki Syndrome 1 | | | MLL4, KMT2D |
| Kennedy Disease (aka | Muscles, brain, | | SBMA/SMAX1/AR |
| Spinobulbar Muscular Atrophy) | nervous system | | |
| Klinefelter syndrome | Various- | | Extra X chromosome in males |
| | particularly | | |
| | those involved | | |
| | in development | | |
| | of male | | |
| | characteristics | | |
| Lafora Disease | Brain, CNS | | EMP2A and EMP2B |
| Leber Congenital Amaurosis | eye | | CRB1, RP12, CORD2, CRD, CRX, |
| | | | IMPDH1, OTX2, AIPL1, CABP4, |
| | | | CCT2, CEP290, CLUAP1, CRB1, |
| | | | CRX, DTHD1, GDF6, GUCY2D, |
| | | | IFT140, IQCB1, KCNJ13, LCA5, |
| | | | LRAT, NMNAT1, PRPH2, RD3, |
| | | | RDH12, RPE65, RP20, RPGRIP1, |
| | | | SPATA7, TULP1, LCA1, LCA4, |
| | | | GUC2D, CORD6, LCA3, |
| Lesch-Nyhan Syndrome | Metabolism | Various - joints, | HPRT1 |
| | disease | cognitive, brain, | |
| | | nervous system | |
| Leukocyte deficiencies and | blood | | ITGB2, CD18, LCAMB, LAD, |
| disorders | | | EIF2B1, EIF2BA, EIF2B2, EIF2B3, |
| | | | EIF2B5, LVWM, CACH, CLE, |
| | | | EIF2B4 |
| Leukemia | Blood | | TAL1, TCL5, SCL, TAL2, FLT3, |
| | | | NBS1, NBS, ZNFN1A1, IK1, LYF1, |
| | | | HOXD4, HOX4B, BCR, CML, PHL, |
| | | | ALL, ARNT, KRAS2, RASK2, |
| | | | GMPS, AF10, ARHGEF12, LARG, |
| | | | KIAA0382, CALM, CLTH, CEBPA, |
| | | | CEBP, CHIC2, BTL, FLT3, KIT, |
| | | | PBT, LPP, NPM1, NUP214, D9S46E, |
| | | | CAN, CAIN, RUNX1, CBFA2, |
| | | | AML1, WHSC1L1, NSD3, FLT3, |
| | | | AF1Q, NPM1, NUMA1, ZNF145, |
| | | | PLZF, PML, MYL, STAT5B, AF10, |
| | | | CALM, CLTH, ARL11, ARLTS1, |
| | | | P2RX7, P2X7, BCR, CML, PHL, |
| | | | ALL, GRAF, NF1, VRNF, WSS, |
| | | | NFNS, PTPN11, PTP2C, SHP2, NS1, |
| | | | BCL2, CCND1, PRAD1, BCL1, |
| | | | TCRA, GATA1, GF1, ERYF1, NFE1, |
| | | | ABL1, NQO1, DIA4, NMOR1, |
| | | | NUP214, D9S46E, CAN, CAIN |
| Limb-girdle muscular dystrophy | muscle | | LGMD |
| diseases | | | |
| Lowe syndrome | brain, eyes, | | OCRL |
| | kidneys | | |
| Lupus glomerulo- nephritis | kidney | | MAPK1 |
| Machado- | Brain, CNS, | | ATX3 |
| Joseph's Disease (also known as | muscle | | |
| Spinocerebellar ataxia Type 3) | | | |
| Macular degeneration | eye | | ABC4, CBC1, CHM1, APOE, |
| | | | C1QTNF5, C2, C3, CCL2, CCR2, |
| | | | CD36, CFB, CFH, CFHR1, CFHR3, |
| | | | CNGB3, CP, CRP, CST3, CTSD, |
| | | | CX3CR1, ELOVL4, ERCC6, FBLN5, |
| | | | FBLN6, FSCN2, HMCN1, HTRA1, |
| | | | IL6, IL8, PLEKHA1, PROM1, |
| | | | PRPH2, RPGR, SERPING1, TCOF1, |
| | | | TIMP3, TLR3 |
| Macular Dystrophy | eye | | BEST1, C1QTNF5, CTNNA1, |
| | | | EFEMP1, ELOVL4, FSCN2, |
| | | | GUCA1B, HMCN1, IMPG1, OTX2, |
| | | | PRDM13, PROM1, PRPH2, RP1L1, |
| | | | TIMP3, ABCA4, CFH, DRAM2, |
| | | | IMG1, MFSD8, ADMD, STGD2, |
| | | | STGD3, RDS, RP7, PRPH, AVMD, |
| | | | AOFMD, VMD2 |
| Malattia Leventinesse | eye | | EFEMP1, FBLN3 |
| Maple Syrup Urine Disease | Metabolism | | BCKDHA, BCKDHB, and DBT |
| | disease | | |
| Marfan syndrome | Connective | Musculoskeletal | FBN1 |
| | tissue | | |
| Maroteaux-Lamy Syndrome (aka | Musculoskeletal | Liver, spleen | ARSB |
| MPS VI) | system, nervous | | |
| | system | | |
| McArdle's Disease (Glycogen | Glycogen | muscle | PYGM |
| Storage Disease Type V) | storage disease | | |
| Medullary cystic kidney disease | kidney | | UMOD, HNFJ, FJHN, MCKD2, |
| | | | ADMCKD2 |
| Metachromatic leukodystrophy | Lysosomal | Nervous system | ARSA |
| | storage disease | | |
| Methylmalonic acidemia (MMA) | Metabolism | | MMAA, MMAB, MUT, MMACHC, |
| | disease | | MMADHC, LMBRD1 |
| Morquio Syndrome (aka MPS IV | Connective | heart | GALNS |
| A and B) | tissue, skin, | | |
| | bone, eyes | | |
| Mucopolysaccharidosis diseases | Lysosomal | | See also Hurler/Scheie syndrome, |
| (Types I H/S, I H, II, III A B and | storage disease - | | Hurler disease, Sanfillipo syndrome, |
| C, I S, IVA and B, IX, VII, and | affects various | | Scheie syndrome, Morquio syndrome, |
| VI) | organs/tissues | | hyaluronidase deficiency, Sly |
| | | | syndrome, and Maroteaux-Lamy |
| | | | syndrome |
| Muscular Atrophy | muscle | | VAPB, VAPC, ALS8, SMN1, SMA1, |
| | | | SMA2, SMA3, SMA4, BSCL2, |
| | | | SPG17, GARS, SMAD1, CMT2D, |
| | | | HEXB, IGHMBP2, SMUBP2, |
| | | | CATF1, SMARD1 |
| Muscular dystrophy | muscle | | FKRP, MDC1C, LGMD2I, LAMA2, |
| | | | LAMM, LARGE, KIAA0609, |
| | | | MDC1D, FCMD, TTID, MYOT, |
| | | | CAPN3, CANP3, DYSF, LGMD2B, |
| | | | SGCG, LGMD2C, DMDA1, SCG3, |
| | | | SGCA, ADL, DAG2, LGMD2D, |
| | | | DMDA2, SGCB, LGMD2E, SGCD, |
| | | | SGD, LGMD2F, CMD1L, TCAP, |
| | | | LGMD2G, CMD1N, TRIM32, HT2A, |
| | | | LGMD2H, FKRP, MDC1C, LGMD2I, |
| | | | TTN, CMD1G, TMD, LGMD2J, |
| | | | POMT1, CAV3, LGMD1C, SEPN1, |
| | | | SELN, RSMD1, PLEC1, PLTN, EBS1 |
| Myotonic dystrophy (Type 1 and | Muscles | Eyes, heart, | CNBP (Type 2) and DMPK (Type 1) |
| Type 2) | | endocrine | |
| Neoplasia | | | PTEN; ATM; ATR; EGFR; ERBB2; |
| | | | ERBB3; ERBB4; |
| | | | Notch1; Notch2; Notch3; Notch4; |
| | | | AKT; AKT2; AKT3; HIF; |
| | | | HIF1a; HIF3a; Met; HRG; Bcl2; |
| | | | PPAR alpha; PPAR |
| | | | gamma; WT1 (Wilms Tumor); FGF |
| | | | Receptor Family |
| | | | members (5 members: 1, 2, 3, 4, 5); |
| | | | CDKN2a; APC; RB |
| | | | (retinoblastoma); MEN1; VHL; |
| | | | BRCA1; BRCA2; AR |
| | | | (Androgen Receptor); TSG101; IGF; |
| | | | IGF Receptor; Igf1 (4 |
| | | | variants); Igf2 (3 variants); Igf 1 |
| | | | Receptor; Igf 2 Receptor; |
| | | | Bax; Bcl2; caspases family (9 |
| | | | members: |
| | | | 1, 2, 3, 4, 6, 7, 8, 9, 12); Kras; Apc |
| Neurofibromatosis (NF) (NF1, | brain, spinal | | NF1, NF2 |
| formerly Recklinghausen's NF, | cord, nerves, | | |
| and NF2) | and skin | | |
| Niemann-Pick Lipidosis (Types | Lysosomal | Various- where | Types A and B: SMPD1; Type C: |
| A, B, and C) | Storage Disease | sphingomyelin | NPC1 or NPC2 |
| | | accumulates, | |
| | | particularly | |
| | | spleen, liver, | |
| | | blood, CNS | |
| Noonan Syndrome | Various - | | PTPN11, SOS1, RAF1 and KRAS |
| | musculoskeletal, | | |
| | heart, eyes, | | |
| | reproductive | | |
| | organs, blood | | |
| Norrie Disease or X-linked | eye | | NDP |
| Familial Exudative | | | |
| Vitreoretinopathy | | | |
| North Carolina Macular | eye | | MCDR1 |
| Dystrophy | | | |
| Osteogenesis imperfecta (OI) | bones, | | COL1A1, COL1A2, CRTAP, P3H |
| (Types I, II, III, IV, V, VI, VII) | musculoskeletal | | |
| Osteopetrosis | bones | | LRP5, BMND1, LRP7, LR3, OPPG, |
| | | | VBCH2, CLCN7, CLC7, OPTA2, |
| | | | OSTM1, GL, TCIRG1, TIRC7, |
| | | | OC116, OPTB1 |
| Patau's Syndrome | Brain, heart, | | Additional copy of chromosome 13 |
| (Trisomy 13) | skeletal system | | |
| Parkinson's disease (PD) | Brain, nervous | | SNCA (PARK1), UCHL1 (PARK 5), |
| | system | | and LRRK2 (PARK8), (PARK3), |
| | | | PARK2, PARK4, PARK7 (PARK7), |
| | | | PINK1 (PARK6); x-Synuclein, DJ-1, |
| | | | Parkin, NR4A2, NURR1, NOT, |
| | | | TINUR, SNCAIP, TBP, SCA17, |
| | | | NCAP, PRKN, PDJ, DBH, NDUFV2 |
| Pattern Dystrophy of the RPE | eye | | RDS/peripherin |
| Phenylketonuria (PKU) | Metabolism | Various due to | PAH, PKU1, QDPR, DHPR, PTS |
| | disorder | build-up of | |
| | | phenylalanine, | |
| | | phenyl ketones | |
| | | in tissues and | |
| | | CNS | |
| Polycystic kidney and hepatic | Kidney, liver | | FCYT, PKHD1, ARPKD, PKD1, |
| disease | | | PKD2, PKD4, PKDTS, PRKCSH, |
| | | | G19P1, PCLD, SEC63 |
| Pompe's Disease | Glycogen | Various - heart, | GAA |
| | storage disease | liver, spleen | |
| Porphyria (actually refers to a | Various- | | ALAD, ALAS2, CPOX, FECH, |
| group of different diseases all | wherever heme | | HMBS, PPOX, UROD, or UROS |
| having a specific heme | precursors | | |
| production process abnormality) | accumulate | | |
| posterior polymorphous corneal | eyes | | TCF4; COL8A2 |
| dystrophy | | | |
| Primary Hyperoxaluria (e.g. type | Various - eyes, | | LDHA (lactate dehydrogenase A) and |
| 1) | heart, kidneys, | | hydroxyacid oxidase 1 (HAO1) |
| | skeletal system | | |
| Primary Open Angle Glaucoma | eyes | | MYOC |
| (POAG) | | | |
| Primary sclerosing cholangitis | Liver, | | TCF4; COL8A2 |
| | gallbladder | | |
| Progeria (also called Hutchinson- | All | | LMNA |
| Gilford progeria syndrome) | | | |
| Prader-Willi Syndrome | Musculoskeletal | | Deletion of region of short arm of |
| | system, brain, | | chromosome 15, including UBE3A |
| | reproductive | | |
| | and endocrine | | |
| | system | | |
| Prostate Cancer | prostate | | HOXB13, MSMB, GPRC6A, TP53 |
| Pyruvate Dehydrogenase | Brain, nervous | | PDHA1 |
| Deficiency | system | | |
| Kidney/Renal carcinoma | kidney | | RLIP76, VEGF |
| Rett Syndrome | Brain | | MECP2, RTT, PPMX, MRX16, |
| | | | MRX79, CDKL5, STK9, MECP2, |
| | | | RTT, PPMX, MRX16, MRX79, x- |
| | | | Synuclein, DJ-1 |
| Retinitis pigmentosa (RP) | eye | | ADIPOR1, ABCA4, AGBL5, |
| | | | ARHGEF18, ARL2BP, ARL3, ARL6, |
| | | | BEST1, BBS1, BBS2, C2ORF71, |
| | | | C8ORF37, CA4, CERKL, CLRN1, |
| | | | CNGA1, CMGB1, CRB1, CRX, |
| | | | CYP4V2, DHDDS, DHX38, EMC1, |
| | | | EYS, FAM161A, FSCN2, GPR125, |
| | | | GUCA1B, HK1, HPRPF3, HGSNAT, |
| | | | IDH3B, IMPDH1, IMPG2, IFT140, |
| | | | IFT172, KLHL7, KIAA1549, KIZ, |
| | | | LRAT, MAK, MERTK, MVK, NEK2, |
| | | | NUROD1, NR2E3, NRL, OFD1, |
| | | | PDE6A, PDE6B, PDE6G, POMGNT1, |
| | | | PRCD, PROM1, PRPF3, PRPF4, |
| | | | PRPF6, PRPF8, PRPF31, PRPH2, |
| | | | RPB3, RDH12, REEP6, RP39, RGR, |
| | | | RHO, RLBP1, ROM1, RP1, RP1L1, |
| | | | RPY, RP2, RP9, RPE65, RPGR, |
| | | | SAMD11, SAG, SEMA4A, SLC7A14, |
| | | | SNRNP200, SPP2, SPATA7, TRNT1, |
| | | | TOPORS, TTC8, TULP1, USH2A, |
| | | | ZFN408, ZNF513, see also |
| | | | 20120204282 |
| Scheie syndrome (also known as | Various- liver, | | IDUA, α-L-iduronidase |
| mucopolysaccharidosis type I | spleen, eye, | | |
| S(MPS I-S)) | joint, heart, | | |
| | brain, skeletal | | |
| Schizophrenia | Brain | | Neuregulin1 (Nrg1); Erb4 (receptor for |
| | | | Neuregulin); |
| | | | Complexin1 (Cplx1); Tph1 |
| | | | Tryptophan hydroxylase; Tph2 |
| | | | Tryptophan hydroxylase 2; Neurexin |
| | | | 1; GSK3; GSK3a; |
| | | | GSK3b; 5-HTT (Slc6a4); COMT; |
| | | | DRD (Drd1a); SLC6A3; DAOA; |
| | | | DTNBP1; Dao (Dao1); TCF4; |
| | | | COL8A2 |
| Secretase Related Disorders | Various | | APH-1 (alpha and beta); PSEN1; |
| | | | NCSTN; PEN-2; Nos1, Parp1, Nat1, |
| | | | Nat2, CTSB, APP, APH1B, PSEN2, |
| | | | PSENEN, BACE1, ITM2B, CTSD, |
| | | | NOTCH1, TNF, INS, DYT10, |
| | | | ADAM17, APOE, ACE, STN, TP53, |
| | | | IL6, NGFR, IL1B, ACHE, CTNNB1, |
| | | | IGF1, IFNG, NRG1, CASP3, MAPK1, |
| | | | CDH1, APBB1, HMGCR, CREB1, |
| | | | PTGS2, HES1, CAT, TGFB1, ENO2, |
| | | | ERBB4, TRAPPC10, MAOB, NGF, |
| | | | MMP12, JAG1, CD40LG, PPARG, |
| | | | FGF2, LRP1, NOTCH4, MAPK8, |
| | | | PREP, NOTCH3, PRNP, CTSG, EGF, |
| | | | REN, CD44, SELP, GHR, ADCYAP1, |
| | | | INSR, GFAP, MMP3, MAPK10, SP1, |
| | | | MYC, CTSE, PPARA, JUN, TIMP1, |
| | | | IL5, IL1A, MMP9, HTR4, HSPG2, |
| | | | KRAS, CYCS, SMG1, IL1R1, |
| | | | PROK1, MAPK3, NTRK1, IL13, |
| | | | MME, TKT, CXCR2, CHRM1, |
| | | | ATXN1, PAWR, NOTCJ2, M6PR, |
| | | | CYP46A1, CSNK1D, MAPK14, |
| | | | PRG2, PRKCA, L1 CAM, CD40, |
| | | | NR1I2, JAG2, CTNND1, CMA1, |
| | | | SORT1, DLK1, THEM4, JUP, CD46, |
| | | | CCL11, CAV3, RNASE3, HSPA8, |
| | | | CASP9, CYP3A4, CCR3, TFAP2A, |
| | | | SCP2, CDK4, JOF1A, TCF7L2, |
| | | | B3GALTL, MDM2, RELA, CASP7, |
| | | | IDE, FANP4, CASK, ADCYAP1R1, |
| | | | ATF4, PDGFA, C21ORF33, SCG5, |
| | | | RMF123, NKFB1, ERBB2, CAV1, |
| | | | MMP7, TGFA, RXRA, STX1A, |
| | | | PSMC4, P2RY2, TNFRSF21, DLG1, |
| | | | NUMBL, SPN, PLSCR1, UBQLN2, |
| | | | UBQLN1, PCSK7, SPON1, SILV, |
| | | | QPCT, HESS, GCC1 |
| Selective IgA Deficiency | Immune system | | Type 1: MSH5; Type 2: TNFRSF13B |
| Severe Combined | Immune system | | JAK3, JAKL, DCLRE1C, ARTEMIS, |
| Immunodeficiency (SCID) and | | | SCIDA, RAG1, RAG2, ADA, PTPRC, |
| SCID-X1, and ADA-SCID | | | CD45, LCA, IL7R, CD3D, T3D, |
| | | | IL2RG, SCIDX1, SCIDX, IMD4, |
| | | | those identified in US Pat. App. Pub. |
| | | | 20110225664, 20110091441, |
| | | | 20100229252, 20090271881 and |
| | | | 20090222937; |
| Sickle cell disease | blood | | HBB, BCL11A, BCL11Ae, cis- |
| | | | regulatory elements of the B-globin |
| | | | locus, HBG 1/2 promoter, HBG distal |
| | | | CCAAT box region between −92 |
| | | | and −130 of the HBG Transcription |
| | | | Start Site, those described in |
| | | | WO2015148863, WO 2013/126794, |
| | | | US Pat. Pub. 20110182867 |
| Sly Syndrome (aka MPS VII) | | | GUSB |
| Spinocerebellar Ataxias (SCA | | | ATXN1, ATXN2, ATX3 |
| types 1, 2, 3, 6, 7, 8, 12 and 17) | | | |
| Sorsby Fundus Dystrophy | eye | | TIMP3 |
| Stargardt disease | eye | | ABCR, ELOVL4, ABCA4, PROM1 |
| Tay-Sachs Disease | Lysosomal | Various - CNS, | HEX-A |
| | Storage disease | brain, eye | |
| Thalassemia (Alpha, Beta, Delta) | blood | | HBA1, HBA2 (Alpha), HBB (Beta), |
| | | | HBB and HBD (delta), LCRB, |
| | | | BCL11A, BCL11Ae, cis-regulatory |
| | | | elements of the B-globin locus, HBG |
| | | | 1/2 promoter, those described in |
| | | | WO2015148860, US Pat. Pub. |
| | | | 20110182867, 2015/148860 |
| Thymic Aplasia (DiGeorge | Immune system, | | deletion of 30 to 40 genes in the |
| Syndrome; 22q11.2 deletion | thymus | | middle of chromosome 22 at |
| syndrome) | | | a location known as 22q11.2, including |
| | | | TBX1, DGCR8 |
| Transthyretin amyloidosis | liver | | TTR (transthyretin) |
| (ATTR) | | | |
| trimethylaminuria | Metabolism | | FMO3 |
| | disease | | |
| Trinucleotide Repeat Disorders | Various | | HTT; SBMA/SMAX1/AR; |
| (generally) | | | FXN/X25 ATX3; |
| | | | ATXN1; ATXN2; |
| | | | DMPK; Atrophin-1 and Atn1 |
| | | | (DRPLA Dx); CBP (Creb-BP - global |
| | | | instability); VLDLR; Atxn7; Atxn10; |
| | | | FEN1, TNRC6A, PABPN1, JPH3, |
| | | | MED15, ATXN1, ATXN3, TBP, |
| | | | CACNA1A, ATXN80S, PPP2R2B, |
| | | | ATXN7, TNRC6B, TNRC6C, CELF3, |
| | | | MAB21L1, MSH2, TMEM185A, |
| | | | SIX5, CNPY3, RAXE, GNB2, RPL14, |
| | | | ATXN8, ISR, TTR, EP400, GIGYF2, |
| | | | OGG1, STC1, CNDP1, C10ORF2, |
| | | | MAML3, DKC1, PAXIP1, CASK, |
| | | | MAPT, SP1, POLG, AFF2, THBS1, |
| | | | TP53, ESR1, CGGBP1, ABT1, KLK3, |
| | | | PRNP, JUN, KCNN3, BAX, FRAXA, |
| | | | KBTBD10, MBNL1, RAD51, |
| | | | NCOA3, ERDA1, TSC1, COMP, |
| | | | GGLC, RRAD, MSH3, DRD2, CD44, |
| | | | CTCF, CCND1, CLSPN, MEF2A, |
| | | | PTPRU, GAPDH, TRIM22, WT1, |
| | | | AHR, GPX1, TPMT, NDP, ARX, |
| | | | TYR, EGR1, UNG, NUMBL, FABP2, |
| | | | EN2, CRYGC, SRP14, CRYGB, |
| | | | PDCD1, HOXA1, ATXN2L, PMS2, |
| | | | GLA, CBL, FTH1, IL12RB2, OTX2, |
| | | | HOXA5, POLG2, DLX2, AHRR, |
| | | | MANF, RMEM158, see also |
| | | | 20110016540 |
| Turner's Syndrome (XO) | Various - | | Monosomy X |
| | reproductive | | |
| | organs, and sex | | |
| | characteristics, | | |
| | vasculature | | |
| Tuberous Sclerosis | CNS, heart, | | TSC1, TSC2 |
| | kidneys | | |
| Usher syndrome (Types I, II, and | Ears, eyes | | ABHD12, CDH23, CIB2, CLRN1, |
| III) | | | DFNB31, GPR98, HARS, MYO7A, |
| | | | PCDH15, USH1C, USH1G, USH2A, |
| | | | USH11A, those described in |
| | | | WO2015134812A1 |
| Velocardiofacial syndrome (aka | Various - | | Many genes are deleted, COM, TBX1, |
| 22q11.2 deletion syndrome, | skeletal, heart, | | and other are associated with |
| DiGeorge syndrome, conotruncal | kidney, immune | | symptoms |
| anomaly face syndrome (CTAF), | system, brain | | |
| autosomal dominant Opitz G/BB | | | |
| syndrome or Cayler cardiofacial | | | |
| syndrome) | | | |
| Von Gierke's Disease (Glycogen | Glycogen | Various - liver, | G6PC and SLC37A4 |
| Storage Disease type I) | Storage disease | kidney | |
| Von Hippel-Lindau Syndrome | Various - cell | CNS, Kidney, | VHL |
| | growth | Eye, visceral | |
| | regulation | organs | |
| | disorder | | |
| Von Willebrand Disease (Types | blood | | VWF |
| I, II and III) | | | |
| Wilson Disease | Various - | Liver, brains, | ATP7B |
| | Copper Storage | eyes, other | |
| | Disease | tissues where | |
| | | copper builds up | |
| Wiskott-Aldrich Syndrome | Immune System | | WAS |
| Xeroderma Pigmentosum | Skin | Nervous system | POLH |
| XXX Syndrome | Endocrine, brain | | X chromosome trisomy |

In an embodiment, the engineered therapeutic polynucleotides of the present invention can be used treat or prevent a disease in a subject by modifying one or more genes associated with one or more cellular functions, such as any one or more of those in Table 6. In an embodiment, the disease is a genetic disease or disorder. In some of embodiments, the engineered therapeutic polynucleotides of the present invention can modify one or more genes or polynucleotides associated with one or more genetic diseases such as any set forth in Table 6.

TABLE 6
Exemplary Genes controlling Cellular Functions

| CELLULAR | |
|---|---|
| FUNCTION | GENES |
| PI3K/AKT | PRKCE; ITGAM; ITGA5; IRAK1; |
| Signaling | PRKAA2; EIF2AK2; PTEN; EIF4E; |
| | PRKCZ; GRK6; MAPK1; TSC1; |
| | PLK1; AKT2; IKBKB; PIK3CA; |
| | CDK8; CDKNIB; NFKB2; BCL2; |
| | PIK3CB; PPP2R1A; MAPK8; |
| | BCL2L1; MAPK3; TSC2; ITGA1; |
| | KRAS; EIF4EBP1; RELA; PRKCD; |
| | NOS3; PRKAA1; MAPK9; CDK2; |
| | PPP2CA; PIM1; ITGB7; YWHAZ; |
| | ILK; TP53; RAF1; IKBKG; RELB; |
| | DYRK1A; CDKN1A; ITGB1; MAP2K2; |
| | JAK1; AKT1; JAK2; PIK3R1; |
| | CHUK; PDPK1; PPP2R5C; CTNNB1; |
| | MAP2K1; NFKB1; PAK3; ITGB3; |
| | CCND1; GSK3A; FRAP1; SFN; |
| | ITGA2; TTK; CSNK1A1; BRAF; |
| | GSK3B; AKT3; FOXO1; SGK; |
| | HSP90AA1; RPS6KB1 |
| ERK/MAPK | PRKCE; ITGAM; ITGA5; HSPB1; |
| Signaling | IRAK1; PRKAA2; EIF2AK2; RAC1; |
| | RAP1A; TLN1; EIF4E; ELK1; |
| | GRK6; MAPK1; RAC2; PLK1; |
| | AKT2; PIK3CA; CDK8; CREB1; |
| | PRKCI; PTK2; FOS; RPS6KA4; |
| | PIK3CB; PPP2R1A; PIK3C3; MAPK8; |
| | MAPK3; ITGA1; ETS1; KRAS; |
| | MYCN; EIF4EBP1; PPARG; PRKCD; |
| | PRKAA1; MAPK9; SRC; CDK2; |
| | PPP2CA; PIM1; PIK3C2A; ITGB7; |
| | YWHAZ; PPP1CC; KSR1; PXN; |
| | RAF1; FYN; DYRK1A; ITGB1; |
| | MAP2K2; PAK4; PIK3R1; STAT3; |
| | PPP2R5C; MAP2K1; PAK3; ITGB3; |
| | ESR1; ITGA2; MYC; TTK; |
| | CSNK1A1; CRKL; BRAF; ATF4; |
| | PRKCA; SRF; STAT1; SGK |
| Glucocorticoid | RAC1; TAF4B; EP300; SMAD2; |
| Receptor | TRAF6; PCAF; ELK1; MAPK1; |
| Signaling | SMAD3; AKT2; IKBKB; NCOR2; |
| | UBE2I; PIK3CA; CREB1; FOS; |
| | HSPA5; NFKB2; BCL2; MAP3K14; |
| | STAT5B; PIK3CB; PIK3C3; |
| | MAPK8; BCL2L1; MAPK3; TSC22D3; |
| | MAPK10; NRIP1; KRAS; MAPK13; |
| | RELA; STAT5A; MAPK9; NOS2A; |
| | PBX1; NR3C1; PIK3C2A; CDKN1C; |
| | TRAF2; SERPINE1; NCOA3; |
| | MAPK14; TNF; RAF1; IKBKG; |
| | MAP3K7; CREBBP; CDKN1A; |
| | MAP2K2; JAK1; IL8; |
| | NCOA2; AKT1; JAK2; |
| | PIK3R1; CHUK; STAT3; MAP2K1; |
| | NFKB1; TGFBR1; |
| | ESR1; SMAD4; CEBPB; JUN; AR; |
| | AKT3; CCL2; MMP1; |
| | STAT1; IL6; HSP90AA1 |
| Axonal | PRKCE; ITGAM; ROCK1; ITGA5; |
| Guidance | CXCR4; ADAM12; |
| Signaling | IGF1; RAC1; RAP1A; EIF4E; |
| | PRKCZ; NRP1; NTRK2; |
| | ARHGEF7; SMO; ROCK2; MAPK1; |
| | PGF; RAC2; |
| | PTPN11; GNAS; AKT2; PIK3CA; |
| | ERBB2; PRKC1; PTK2; |
| | CFL1; GNAQ; PIK3CB; CXCL12; |
| | PIK3C3; WNT11; |
| | PRKD1; GNB2L1; ABL1; MAPK3; |
| | ITGA1; KRAS; RHOA; |
| | PRKCD; PIK3C2A; ITGB7; GLI2; |
| | PXN; VASP; RAF1; |
| | FYN; ITGB1; MAP2K2; PAK4; |
| | ADAM17; AKT1; PIK3R1; |
| | GLI1; WNT5A; ADAM10; MAP2K1; |
| | PAK3; ITGB3; |
| | CDC42; VEGFA; ITGA2; EPHA8; |
| | CRKL; RND1; GSK3B; |
| | AKT3; PRKCA |
| Ephrin | PRKCE; ITGAM; ROCK1; ITGA5; |
| Receptor | CXCR4; IRAK1; |
| Signaling | PRKAA2; EIF2AK2; RAC1; RAP1A; |
| Actin | GRK6; ROCK2; |
| Cytoskeleton | MAPK1; PGF; RAC2; PTPN11; |
| Signaling | GNAS; PLK1; AKT2; |
| | DOK1; CDK8; CREB1; PTK2; |
| | CFL1; GNAQ; MAP3K14; |
| | CXCL12; MAPK8; GNB2L1; ABL1; |
| | MAPK3; ITGA1; |
| | KRAS; RHOA; PRKCD; PRKAA1; |
| | MAPK9; SRC; CDK2; |
| | PIM1; ITGB7; PXN; RAF1; |
| | FYN; DYRK1A; ITGB1; |
| | MAP2K2; PAK4; AKT1; JAK2; |
| | STAT3; ADAM10; |
| | MAP2K1; PAK3; ITGB3; CDC42; |
| | VEGFA; ITGA2; |
| | EPHA8; TTK; CSNK1A1; CRKL; |
| | BRAF; PTPN13; ATF4; |
| | AKT3; SGK |
| | ACTN4; PRKCE; ITGAM; ROCK1; |
| | ITGA5; IRAK1; |
| | PRKAA2; EIF2AK2; RAC1; INS; |
| | ARHGEF7; GRK6; |
| | ROCK2; MAPK1; RAC2; PLK1; |
| | AKT2; PIK3CA; CDK8; |
| | PTK2; CFL1; PIK3CB; MYH9; |
| | DIAPH1; PIK3C3; MAPK8; |
| | F2R; MAPK3; SLC9A1; ITGA1; |
| | KRAS; RHOA; PRKCD; |
| | PRKAA1; MAPK9; CDK2; PIM1; |
| | PIK3C2A; ITGB7; |
| | PPP1CC; PXN; VIL2; RAF1; |
| | GSN; DYRK1A; ITGB1; |
| | MAP2K2; PAK4; PIP5K1A; PIK3R1; |
| | MAP2K1; PAK3; |
| | ITGB3; CDC42; APC; ITGA2; |
| | TTK; CSNK1A1; CRKL; |
| | BRAF; VAV3; SGK |
| Huntington's | PRKCE; IGF1; EP300; RCOR1; |
| Disease | PRKCZ; HDAC4; TGM2; |
| Signaling | MAPK1; CAPNS1; AKT2; EGFR; |
| | NCOR2; SP1; CAPN2; |
| | PIK3CA; HDAC5; CREB1; PRKC1; |
| | HSPA5; REST; |
| | GNAQ; PIK3CB; PIK3C3; MAPK8; |
| | IGF1R; PRKD1; |
| | GNB2L1; BCL2L1; CAPN1; MAPK3; |
| | CASP8; HDAC2; |
| | HDAC7A; PRKCD; HDAC11; MAPK9; |
| | HDAC9; PIK3C2A; |
| | HDAC3; TP53; CASP9; CREBBP; |
| | AKT1; PIK3R1; |
| | PDPK1; CASP1; APAF1; FRAP1; |
| | CASP2; JUN; BAX; |
| | ATF4; AKT3; PRKCA; CLTC; |
| | SGK; HDAC6; CASP3 |
| Apoptosis | PRKCE; ROCK1; BID; IRAK1; |
| Signaling | PRKAA2; EIF2AK2; BAK1; |
| | BIRC4; GRK6; MAPK1; CAPNS1; |
| | PLK1; AKT2; IKBKB; |
| | CAPN2; CDK8; FAS; NFKB2; |
| | BCL2; MAP3K14; MAPK8; |
| | BCL2L1; CAPN1; MAPK3; CASP8; |
| | KRAS; RELA; |
| | PRKCD; PRKAA1; MAPK9; CDK2; |
| | PIM1; TP53; TNF; |
| | RAF1; IKBKG; RELB; CASP9; |
| | DYRK1A; MAP2K2; |
| | CHUK; APAF1; MAP2K1; NFKB1; |
| | PAK3; LMNA; CASP2; |
| | BIRC2; TTK; CSNK1A1; BRAF; |
| | BAX; PRKCA; SGK; |
| | CASP3; BIRC3; PARP1 |
| B Cell | RAC1; PTEN; LYN; ELK1; |
| Receptor | MAPK1; RAC2; PTPN11; |
| Signaling | AKT2; IKBKB; PIK3CA; CREB1; |
| | SYK; NFKB2; CAMK2A; |
| | MAP3K14; PIK3CB; PIK3C3; MAPK8; |
| | BCL2L1; ABL1; |
| | MAPK3; ETS1; KRAS; MAPK13; |
| | RELA; PTPN6; MAPK9; |
| | EGR1; PIK3C2A; BTK; MAPK14; |
| | RAF1; IKBKG; RELB; |
| | MAP3K7; MAP2K2; AKT1; PIK3R1; |
| | CHUK; MAP2K1; |
| | NFKB1; CDC42; GSK3A; FRAP1; |
| | BCL6; BCL10; JUN; |
| | GSK3B; ATF4; AKT3; VAV3; |
| | RPS6KB1 |
| Leukocyte | ACTN4; CD44; PRKCE; ITGAM; |
| Extravasation | ROCK1; CXCR4; CYBA; |
| Signaling | RAC1; RAP1A; PRKCZ; ROCK2; |
| | RAC2; PTPN11; |
| | MMP14; PIK3CA; PRKC1; PTK2; |
| | PIK3CB; CXCL12; |
| | PIK3C3; MAPK8; PRKD1; ABL1; |
| | MAPK10; CYBB; |
| | MAPK13; RHOA; PRKCD; MAPK9; |
| | SRC; PIK3C2A; BTK; |
| | MAPK14; NOX1; PXN; VIL2; |
| | VASP; ITGB1; MAP2K2; |
| | CTNND1; PIK3R1; CTNNB1; CLDN1; |
| | CDC42; F11R; ITK; |
| | CRKL; VAV3; CTTN; PRKCA; |
| | MMP1; MMP9 |
| Integrin | ACTN4; ITGAM; ROCK1; ITGA5; |
| Signaling | RAC1; PTEN; RAP1A; |
| | TLN1; ARHGEF7; MAPK1; RAC2; |
| | CAPNS1; AKT2; |
| | CAPN2; PIK3CA; PTK2; PIK3CB; |
| | PIK3C3; MAPK8; |
| | CAV1; CAPN1; ABL1; MAPK3; |
| | ITGA1; KRAS; RHOA; |
| | SRC; PIK3C2A; ITGB7; PPP1CC; |
| | ILK; PXN; VASP; |
| | RAF1; FYN; ITGB1; MAP2K2; |
| | PAK4; AKT1; PIK3R1; |
| | TNK2; MAP2K1; PAK3; ITGB3; |
| | CDC42; RND3; ITGA2; |
| | CRKL; BRAF; GSK3B; AKT3 |
| Acute Phase | IRAK1; SOD2; MYD88; TRAF6; |
| Response | ELK1; MAPK1; PTPN11; |
| Signaling | AKT2; IKBKB; PIK3CA; FOS; |
| | NFKB2; MAP3K14; |
| | PIK3CB; MAPK8; RIPK1; MAPK3; |
| | IL6ST; KRAS; |
| | MAPK13; IL6R; RELA; SOCS1; |
| | MAPK9; FTL; NR3C1; |
| | TRAF2; SERPINE1; MAPK14; TNF; |
| | RAF1; PDK1; |
| | IKBKG; RELB; MAP3K7; MAP2K2; |
| | AKT1; JAK2; PIK3R1; |
| | CHUK; STAT3; MAP2K1; NFKB1; |
| | FRAP1; CEBPB; JUN; |
| | AKT3; IL1R1; IL6 |
| PTEN | ITGAM; ITGA5; RAC1; PTEN; |
| Signaling | PRKCZ; BCL2L11; |
| | MAPK1; RAC2; AKT2; EGFR; |
| | IKBKB; CBL; PIK3CA; |
| | CDKN1B; PTK2; NFKB2; BCL2; |
| | PIK3CB; BCL2L1; |
| | MAPK3; ITGA1; KRAS; ITGB7; |
| | ILK; PDGFRB; INSR; |
| | RAF1; IKBKG; CASP9; CDKN1A; |
| | ITGB1; MAP2K2; |
| | AKT1; PIK3R1; CHUK; PDGFRA; |
| | PDPK1; MAP2K1; |
| | NFKB1; ITGB3; CDC42; CCND1; |
| | GSK3A; ITGA2; |
| | GSK3B; AKT3; FOXO1; CASP3; |
| | RPS6KB1 |
| p53 | PTEN; EP300; BBC3; PCAF; |
| Signaling | FASN; BRCA1; GADD45A; |
| | BIRC5; AKT2; PIK3CA; CHEK1; |
| | TP53INP1; BCL2; |
| | PIK3CB; PIK3C3; MAPK8; THBS1; |
| | ATR; BCL2L1; E2F1; |
| | PMAIP1; CHEK2; TNFRSF10B; TP73; |
| | RB1; HDAC9; |
| | CDK2; PIK3C2A; MAPK14; TP53; |
| | LRDD; CDKN1A; |
| | HIPK2; AKT1; PIK3R1; RRM2B; |
| | APAF1; CTNNB1; |
| | SIRT1; CCND1; PRKDC; ATM; |
| | SFN; CDKN2A; JUN; |
| | SNAI2; GSK3B; BAX; AKT3 |
| Aryl | HSPB1; EP300; FASN; TGM2; |
| Hydrocarbon | RXRA; MAPK1; NQO1; |
| Receptor | NCOR2; SP1; ARNT; CDKN1B; |
| Signaling | FOS; CHEK1; |
| | SMARCA4; NFKB2; MAPK8; ALDH1A1; |
| | ATR; E2F1; |
| | MAPK3; NRIP1; CHEK2; RELA; |
| | TP73; GSTP1; RB1; |
| | SRC; CDK2; AHR; NFE2L2; |
| | NCOA3; TP53; TNF; |
| | CDKN1A; NCOA2; APAF1; NFKB1; |
| | CCND1; ATM; ESR1; |
| | CDKN2A; MYC; JUN; ESR2; |
| | BAX; IL6; CYP1B1; |
| | HSP90AA1 |
| Xenobiotic | PRKCE; EP300; PRKCZ; RXRA; |
| Metabolism | MAPK1; NQO1; |
| Signaling | NCOR2; PIK3CA; ARNT; PRKCI; |
| | NFKB2; CAMK2A; |
| | PIK3CB; PPP2R1A; PIK3C3; MAPK8; |
| | PRKD1; ALDH1A1; MAPK3; NRIP1; |
| | KRAS; MAPK13; PRKCD; GSTP1; |
| | MAPK9; NOS2A; ABCB1; AHR; |
| | PPP2CA; FTL; NFE2L2; PIK3C2A; |
| | PPARGC1A; MAPK14; TNF; RAF1; |
| | CREBBP; MAP2K2; PIK3R1; PPP2R5C; |
| | MAP2K1; NFKB1; KEAP1; PRKCA; |
| | EIF2AK3; IL6; CYP1B1; |
| | HSP90AA1 |
| SAPK/JNK | PRKCE; IRAK1; PRKAA2; EIF2AK2; |
| Signaling | RAC1; ELK1; GRK6; MAPK1; |
| | GADD45A; RAC2; PLK1; AKT2; |
| | PIK3CA; FADD; CDK8; PIK3CB; |
| | PIK3C3; MAPK8; RIPK1; |
| | GNB2L1; IRS1; MAPK3; MAPK10; |
| | DAXX; KRAS; PRKCD; PRKAA1; |
| | MAPK9; CDK2; PIM1; PIK3C2A; |
| | TRAF2; TP53; LCK; MAP3K7; |
| | DYRK1A; MAP2K2; PIK3R1; MAP2K1; |
| | PAK3; CDC42; JUN; TTK; CSNK1A1; |
| | CRKL; BRAF; SGK |
| PPAr/RXR | PRKAA2; EP300; INS; SMAD2; |
| Signaling | TRAF6; PPARA; FASN; RXRA; |
| | MAPK1; SMAD3; GNAS; IKBKB; |
| | NCOR2; ABCA1; GNAQ; NFKB2; |
| | MAP3K14; STAT5B; MAPK8; |
| | IRS1; MAPK3; KRAS; RELA; |
| | PRKAA1; PPARGC1A; NCOA3; |
| | MAPK14; INSR; RAF1; |
| | IKBKG; RELB; MAP3K7; |
| | CREBBP; MAP2K2; JAK2; CHUK; |
| | MAP2K1; NFKB1; TGFBR1; SMAD4; |
| | JUN; IL1R1; PRKCA; IL6; |
| | HSP90AA1; ADIPOQ |
| NF-KB | IRAK1; EIF2AK2; EP300; INS; |
| Signaling | MYD88; PRKCZ; TRAF6; |
| | TBK1; AKT2; EGFR; IKBKB; |
| | PIK3CA; BTRC; NFKB2; |
| | MAP3K14; PIK3CB; PIK3C3; |
| | MAPK8; RIPK1; HDAC2; |
| | KRAS; RELA; PIK3C2A; TRAF2; |
| | TLR4; PDGFRB; TNF; |
| | INSR; LCK; IKBKG; RELB; |
| | MAP3K7; CREBBP; AKT1; |
| | PIK3R1; CHUK; PDGFRA; NFKB1; |
| | TLR2; BCL10; GSK3B; AKT3; |
| | TNFAIP3; IL1R1 |
| Neuregulin | ERBB4; PRKCE; ITGAM; ITGA5; |
| Signaling | PTEN; PRKCZ; ELK1; MAPK1; |
| | PTPN11; AKT2; EGFR; ERBB2; |
| | PRKCI; CDKN1B; STAT5B; PRKD1; |
| | MAPK3; ITGA1; KRAS; PRKCD; |
| | STAT5A; SRC; ITGB7; RAF1; |
| | ITGB1; MAP2K2; ADAM17; AKT1; |
| | PIK3R1; PDPK1; MAP2K1; ITGB3; |
| | EREG; FRAP1; PSEN1; ITGA2; |
| | MYC; NRG1; CRKL; AKT3; |
| | PRKCA; HSP90AA1; RPS6KB1 |
| Wnt & Beta | CD44; EP300; LRP6; DVL3; |
| catenin | CSNK1E; GJA1; SMO; AKT2; |
| Signaling | PIN1; CDH1; BTRC; GNAQ; |
| | MARK2; PPP2R1A; WNT11; SRC; |
| | DKK1; PPP2CA; SOX6; SFRP2; |
| | ILK; LEF1; SOX9; TP53; |
| | MAP3K7; CREBBP; TCF7L2; AKT1; |
| | PPP2R5C; WNT5A; LRP5; CTNNB1; |
| | TGFBR1; CCND1; GSK3A; DVL1; |
| | APC; CDKN2A; MYC; CSNK1A1; |
| | GSK3B; AKT3; SOX2 |
| Insulin | PTEN; INS; EIF4E; PTPN1; |
| Receptor | PRKCZ; MAPK1; TSC1; PTPN11; |
| Signaling | AKT2; CBL; PIK3CA; |
| | PRKCI; PIK3CB; PIK3C3; |
| | MAPK8; IRS1; MAPK3; TSC2; |
| | KRAS; EIF4EBP1; SLC2A4; |
| | PIK3C2A ;PPP1CC; INSR; |
| | RAF1; FYN; MAP2K2; JAK1; |
| | AKT1; JAK2; PIK3R1; PDPK1; |
| | MAP2K1; GSK3A; FRAP1; CRKL; |
| | GSK3B; AKT3; FOXO1; SGK; |
| | RPS6KB1 |
| IL-6 | HSPB1; TRAF6; MAPKAPK2; ELK1; |
| Signaling | MAPK1; PTPN11; IKBKB; FOS; |
| | NFKB2; MAP3K14; MAPK8; MAPK3; |
| | MAPK10; IL6ST; KRAS; MAPK13; |
| | IL6R; RELA; SOCS1; MAPK9; |
| | ABCB1; TRAF2; MAPK14; TNF; |
| | RAF1; IKBKG; RELB; MAP3K7; |
| | MAP2K2; IL8; JAK2; CHUK; |
| | STAT3; MAP2K1; NFKB1; CEBPB; |
| | JUN; IL1R1; SRF; IL6 |
| Hepatic | PRKCE; IRAK1; INS; MYD88; |
| Cholestasis | PRKCZ; TRAF6; PPARA; RXRA; |
| | IKBKB; PRKCI; NFKB2; |
| | MAP3K14; MAPK8; PRKD1; |
| | MAPK10; RELA; PRKCD; |
| | MAPK9; ABCB1; TRAF2; TLR4; |
| | TNF; INSR; IKBKG; RELB; |
| | MAP3K7; IL8; CHUK; NR1H2; |
| | TJP2; NFKB1; ESR1; SREBF1; |
| | FGFR4; JUN; IL1R1; PRKCA; |
| | IL6 |
| IGF-1 | IGF1; PRKCZ; ELK1; MAPK1; |
| Signaling | PTPN11; NEDD4; AKT2; |
| | PIK3CA; PRKCI; PTK2; FOS; |
| | PIK3CB; PIK3C3; MAPK8; |
| | IGF1R; IRS1; MAPK3; IGFBP7; |
| | KRAS; PIK3C2A; YWHAZ; PXN; |
| | RAF1; CASP9; MAP2K2; AKT1; |
| | PIK3R1; PDPK1; MAP2K1; IGFBP2; |
| | SFN; JUN; CYR61; AKT3; |
| | FOXO1; SRF; CTGF; RPS6KB1 |
| NRF2-mediated | PRKCE; EP300; SOD2; PRKCZ; |
| Oxidative | MAPK1; SQSTM1; NQO1; PIK3CA; |
| Stress | PRKC1; FOS; PIK3CB; PIK3C3; |
| Response | MAPK8; PRKD1; MAPK3; KRAS; |
| | PRKCD; GSTP1; MAPK9; FTL; |
| | NFE2L2; PIK3C2A; MAPK14; RAF1; |
| | MAP3K7; CREBBP; MAP2K2; AKT1; |
| | PIK3R1; MAP2K1; PPIB; JUN; |
| | KEAP1; GSK3B; ATF4; PRKCA; |
| | EIF2AK3; HSP90AA1 |
| Hepatic | EDN1; IGF1; KDR; FLT1; |
| Fibrosis/Hepatic | SMAD2; FGFR1; MET; PGF; |
| Stellate Cell | SMAD3; EGFR; FAS; CSF1; |
| Activation | NFKB2; BCL2; MYH9; IGF1R; |
| | IL6R; RELA; TLR4; PDGFRB; |
| | TNF; RELB; IL8; PDGFRA; |
| | NFKB1; TGFBR1; SMAD4; |
| | VEGFA; BAX; IL1R1; CCL2; |
| | HGF; MMP1; STAT1; IL6; |
| | CTGF; MMP9 |
| PPAR | EP300; INS; TRAF6; PPARA; |
| Signaling | RXRA; MAPK1; IKBKB; NCOR2; |
| | FOS; NFKB2; MAP3K14; |
| | STAT5B; MAPK3; NRIP1; KRAS; |
| | PPARG; RELA; STAT5A; TRAF2; |
| | PPARGC1A; PDGFRB; TNF; INSR; |
| | RAF1; IKBKG; RELB; MAP3K7; |
| | CREBBP; MAP2K2; CHUK; PDGFRA; |
| | MAP2K1; NFKB1; JUN; IL1R1; |
| | HSP90AA1 |
| Fc Epsilon | PRKCE; RAC1; PRKCZ; LYN; |
| RI Signaling | MAPK1; RAC2; PTPN11; |
| | AKT2; PIK3CA; SYK; PRKCI; |
| | PIK3CB; PIK3C3; MAPK8; |
| | PRKD1; MAPK3; MAPK10; KRAS; |
| | MAPK13; PRKCD; MAPK9; PIK3C2A; |
| | BTK; MAPK14; TNF; RAF1; FYN; |
| | MAP2K2; AKT1; PIK3R1; PDPK1; |
| | MAP2K1; AKT3; VAV3; PRKCA |
| G-Protein | PRKCE; RAP1A; RGS16; MAPK1; |
| Coupled | GNAS; AKT2; IKBKB; PIK3CA; |
| Receptor | CREB1; GNAQ; NFKB2; CAMK2A; |
| Signaling | PIK3CB; PIK3C3; MAPK3; KRAS; |
| | RELA; SRC; PIK3C2A; RAF1; |
| | IKBKG; RELB; FYN; MAP2K2; |
| | AKT1; PIK3R1; CHUK; PDPK1; |
| | STAT3; MAP2K1; NFKB1; BRAF; |
| | ATF4; AKT3; PRKCA |
| Inositol | PRKCE; IRAK1; PRKAA2; EIF2AK2; |
| Phosphate | PTEN; GRK6; |
| Metabolism | MAPK1; PLK1; AKT2; PIK3CA; |
| | CDK8; PIK3CB; PIK3C3; |
| | MAPK8; MAPK3; PRKCD; PRKAA1; |
| | MAPK9; CDK2; |
| | PIM1; PIK3C2A; DYRK1A; MAP2K2; |
| | PIP5K1A; PIK3R1; |
| | MAP2K1; PAK3; ATM; TTK; |
| | CSNK1A1; BRAF; SGK |
| PDGF | EIF2AK2; ELK1; ABL2; MAPK1; |
| Signaling | PIK3CA; FOS; PIK3CB; |
| | PIK3C3; MAPK8; CAV1; ABL1; |
| | MAPK3; KRAS; SRC; PIK3C2A; |
| | PDGFRB; RAF1; MAP2K2; |
| | JAK1; JAK2; PIK3R1; PDGFRA; |
| | STAT3; SPHK1; MAP2K1; MYC; |
| | JUN; CRKL; PRKCA; SRF; |
| | STAT1; SPHK2 |
| VEGF | ACTN4; ROCK1; KDR; FLT1; |
| Signaling | ROCK2; MAPK1; PGF; AKT2; |
| | PIK3CA; ARNT; PTK2; BCL2; |
| | PIK3CB; PIK3C3; BCL2L1; |
| | MAPK3; KRAS; HIF1A; |
| | NOS3; PIK3C2A; PXN; |
| | RAF1; MAP2K2; ELAVL1; AKT1; |
| | PIK3R1; MAP2K1; SFN; |
| | VEGFA; AKT3; FOXO1; PRKCA |
| Natural | PRKCE; RAC1; PRKCZ; MAPK1; |
| Killer Cell | RAC2; PTPN11; |
| Signaling | KIR2DL3; AKT2; PIK3CA; SYK; |
| | PRKCI; PIK3CB; |
| | PIK3C3; PRKD1; MAPK3; KRAS; |
| | PRKCD; PTPN6; |
| | PIK3C2A; LCK; RAF1; FYN; |
| | MAP2K2; PAK4; AKT1; |
| | PIK3R1; MAP2K1; PAK3; AKT3; |
| | VAV3; PRKCA |
| Cell Cycle: | HDAC4; SMAD3; SUV39H1; HDAC5; |
| G1/S | CDKN1B; BTRC; ATR; ABL1; |
| Checkpoint | E2F1; HDAC2; HDAC7A; RB1; |
| Regulation | HDAC11; HDAC9; CDK2; E2F2; |
| | HDAC3; TP53; CDKN1A; CCND1; |
| | E2F4; ATM; RBL2; SMAD4; |
| | CDKN2A; MYC; NRG1; GSK3B; |
| | RBL1; HDAC6 |
| T Cell | RAC1; ELK1; MAPK1; IKBKB; |
| Receptor | CBL; PIK3CA; FOS; NFKB2; |
| Signaling | PIK3CB; PIK3C3; MAPK8; |
| | MAPK3; KRAS; RELA; |
| | PIK3C2A; BTK; LCK; RAF1; |
| | IKBKG; RELB; FYN; MAP2K2; |
| | PIK3R1; CHUK; MAP2K1; |
| | NFKB1; ITK; BCL10; JUN; |
| | VAV3 |
| Death | CRADD; HSPB1; BID; BIRC4; |
| Receptor | TBK1; IKBKB; FADD; FAS; |
| Signaling | NFKB2; BCL2; MAP3K14; |
| | MAPK8; RIPK1; CASP8; |
| | DAXX; TNFRSF10B; RELA; |
| | TRAF2; TNF; IKBKG; RELB; |
| | CASP9; CHUK; APAF1; NFKB1; |
| | CASP2; BIRC2; CASP3; BIRC3 |
| FGF | RAC1; FGFR1; MET; MAPKAPK2; |
| Signaling | MAPK1; PTPN11; AKT2; PIK3CA; |
| | CREB1; PIK3CB; PIK3C3; MAPK8; |
| | MAPK3; MAPK13; PTPN6; PIK3C2A; |
| | MAPK14; RAF1; AKT1; PIK3R1; |
| | STAT3; MAP2K1; FGFR4; CRKL; |
| | ATF4; AKT3; PRKCA; HGF |
| GM-CSF | LYN; ELK1; MAPK1; PTPN11; |
| Signaling | AKT2; PIK3CA; CAMK2A; |
| | STAT5B; PIK3CB; PIK3C3; GNB2L1; |
| | BCL2L1; MAPK3; ETS1; KRAS; |
| | RUNX1; PIM1; PIK3C2A; RAF1; |
| | MAP2K2; AKT1; JAK2; PIK3R1; |
| | STAT3; MAP2K1; CCND1; AKT3; |
| | STAT1 |
| Amyotrophic | BID; IGF1; RAC1; BIRC4; |
| Lateral | PGF; CAPNS1; CAPN2; PIK3CA; |
| Sclerosis | BCL2; PIK3CB; PIK3C3; BCL2L1; |
| Signaling | CAPN1; PIK3C2A; TP53; CASP9; |
| | PIK3R1; RAB5A; CASP1; |
| | APAF1; VEGFA; BIRC2; BAX; |
| | AKT3; CASP3; BIRC3 |
| JAK/Stat | PTPN1; MAPK1; PTPN11; AKT2; |
| Signaling | PIK3CA; STAT5B; PIK3CB; |
| | PIK3C3; MAPK3; KRAS; |
| | SOCS1; STAT5A; PTPN6; |
| | PIK3C2A; RAF1; CDKN1A; |
| | MAP2K2; JAK1; AKT1; JAK2; |
| | PIK3R1; STAT3; MAP2K1; FRAP1; |
| | AKT3; STAT1 |
| Nicotinate | PRKCE; IRAK1; PRKAA2; EIF2AK2; |
| and | GRK6; MAPK1; PLK1; AKT2; |
| Nicotinamide | CDK8; MAPK8; MAPK3; PRKCD; |
| Metabolism | PRKAA1; PBEF1; MAPK9; CDK2; |
| | PIM1; DYRK1A; MAP2K2; |
| | MAP2K1; PAK3; NT5E; TTK; |
| | CSNK1A1; BRAF; SGK |
| Chemokine | CXCR4; ROCK2; MAPK1; PTK2; |
| Signaling | FOS; CFL1; GNAQ; CAMK2A; |
| | CXCL12; MAPK8; MAPK3; |
| | KRAS; MAPK13; RHOA; CCR3; |
| | SRC; PPP1CC; MAPK14; NOX1; |
| | RAF1; MAP2K2; MAP2K1; JUN; |
| | CCL2; PRKCA |
| IL-2 | ELK1; MAPK1; PTPN11; AKT2; |
| Signaling | PIK3CA; SYK; FOS; STAT5B; |
| | PIK3CB; PIK3C3; MAPK8; |
| | MAPK3; KRAS; SOCS1; STAT5A; |
| | PIK3C2A; LCK; RAF1; MAP2K2; |
| | JAK1; AKT1; PIK3R1; MAP2K1; |
| | JUN; AKT3 |
| Synaptic | PRKCE; IGF1; PRKCZ; PRDX6; |
| Long Term | LYN; MAPK1; GNAS; |
| Depression | PRKCI; GNAQ; PPP2R1A; IGF1R; |
| | PRKD1; MAPK3; KRAS; GRN; |
| | PRKCD; NOS3; NOS2A; PPP2CA; |
| | YWHAZ; RAF1; MAP2K2; PPP2R5C; |
| | MAP2K1; PRKCA |
| Estrogen | TAF4B; EP300; CARM1; PCAF; |
| Receptor | MAPK1; NCOR2; SMARCA4; MAPK3; |
| Signaling | NRIP1; KRAS; SRC; NR3C1; |
| | HDAC3; PPARGC1A; RBM9; NCOA3; |
| | RAF1; CREBBP; MAP2K2; NCOA2; |
| | MAP2K1; PRKDC; ESR1; ESR2 |
| Protein | TRAF6; SMURF1; BIRC4; BRCA1; |
| Ubiquitination | UCHL1; NEDD4; CBL; UBE2I; |
| Pathway | BTRC; HSPA5; USP7; USP10; |
| | FBXW7; USP9X; STUB1; USP22; |
| | B2M; BIRC2; PARK2; USP8; |
| | USP1; VHL; HSP90AA1; BIRC3 |
| IL-10 | TRAF6; CCR1; ELK1; IKBKB; |
| Signaling | SP1; FOS; NFKB2; MAP3K14; |
| | MAPK8; MAPK13; RELA; MAPK14; |
| | TNF; IKBKG; RELB; MAP3K7; |
| | JAK1; CHUK; STAT3; NFKB1; |
| | JUN; ILIR1; IL6 |
| VDR/RXR | PRKCE; EP300; PRKCZ; RXRA; |
| Activation | GADD45A; HES1; NCOR2; SP1; |
| | PRKCI; CDKN1B; PRKD1; PRKCD; |
| | RUNX2; KLF4; YY1; NCOA3; |
| | CDKN1A; NCOA2; SPP1; |
| | LRP5; CEBPB; FOXO1; PRKCA |
| TGF-beta | EP300; SMAD2; SMURF1; MAPK1; |
| Signaling | SMAD3; SMAD1; FOS; MAPK8; |
| | MAPK3; KRAS; MAPK9; RUNX2; |
| | SERPINE1; RAF1; MAP3K7; CREBBP; |
| | MAP2K2; MAP2K1; TGFBR1; SMAD4; |
| | JUN; SMAD5 |
| Toll-like | IRAK1; EIF2AK2; MYD88; TRAF6; |
| Receptor | PPARA; ELK1; IKBKB; FOS; |
| Signaling | NFKB2; MAP3K14; MAPK8; MAPK13; |
| | RELA; TLR4; MAPK14; IKBKG; |
| | RELB; MAP3K7; CHUK; NFKB1; |
| | TLR2; JUN |
| p38 MAPK | HSPB1; IRAK1; TRAF6; MAPKAPK2; |
| Signaling | ELK1; FADD; FAS; CREB1; |
| | DDIT3; RPS6KA4; DAXX; MAPK13; |
| | TRAF2; MAPK14; TNF; MAP3K7; |
| | TGFBR1; MYC; ATF4; IL1R1; |
| | SRF; STAT1 |
| Neurotrophin/TRK | NTRK2; MAPK1; PTPN11; PIK3CA; |
| Signaling | CREB1; FOS; PIK3CB; PIK3C3; |
| | MAPK8; MAPK3; KRAS; PIK3C2A; |
| | RAF1; MAP2K2; AKT1; PIK3R1; |
| | PDPK1; MAP2K1; CDC42; JUN; |
| | ATF4 |
| FXR/RXR | INS; PPARA; FASN; RXRA; |
| Activation | AKT2; SDC1; MAPK8; APOB; |
| | MAPK10; PPARG; MTTP; MAPK9; |
| | PPARGC1A; TNF; CREBBP; AKT1; |
| | SREBF1; FGFR4; AKT3; FOXO1 |
| Synaptic | PRKCE; RAP1A; EP300; PRKCZ; |
| Long Term | MAPK1; CREB1; PRKCI; GNAQ; |
| Potentiation | CAMK2A; PRKD1; MAPK3; KRAS; |
| | PRKCD; PPP1CC; RAF1; CREBBP; |
| | MAP2K2; MAP2K1; ATF4; PRKCA |
| Calcium | RAP1A; EP300; HDAC4; MAPK1; |
| Signaling | HDAC5; CREB1; CAMK2A; MYH9; |
| | MAPK3; HDAC2; HDAC7A; HDAC11; |
| | HDAC9; HDAC3; CREBBP; CALR; |
| | CAMKK2; ATF4; HDAC6 |
| EGF Signaling | ELK1; MAPK1; EGFR; PIK3CA; |
| | FOS; PIK3CB; PIK3C3; MAPK8; |
| | MAPK3; PIK3C2A; RAF1; JAK1; |
| | PIK3R1; STAT3; MAP2K1; JUN; |
| | PRKCA; SRF; STAT1 |
| Hypoxia Signaling | EDN1; PTEN; EP300; NQO1; |
| in the | UBE2I; CREB1; ARNT; HIF1A; |
| Cardiovascular | SLC2A4; NOS3; TP53; LDHA; |
| System | AKT1; ATM; VEGFA; JUN; |
| | ATF4; VHL; HSP90AA1 |
| LPS/IL-1 Mediated | IRAK1; MYD88; TRAF6; PPARA; |
| Inhibition of | RXRA; ABCA1; MAPK8; ALDH1A1; |
| RXR Function | GSTP1; MAPK9; ABCB1; TRAF2; |
| | TLR4; TNF; MAP3K7; NR1H2; |
| | SREBF1; JUN; IL1R1 |
| LXR/RXR Activation | FASN; RXRA; NCOR2; ABCA1; |
| | NFKB2; IRF3; RELA; NOS2A; |
| | TLR4; TNF; RELB; LDLR; |
| | NR1H2; NFKB1; SREBF1; IL1R1; |
| | CCL2; IL6; MMP9 |
| Amyloid | PRKCE; CSNK1E; MAPK1; CAPNS1; |
| Processing | AKT2; CAPN2; CAPN1; MAPK3; |
| | MAPK13; MAPT; MAPK14; AKT1; |
| | PSEN1; CSNK1A1; GSK3B; AKT3; |
| | APP |
| IL-4 Signaling | AKT2; PIK3CA; PIK3CB; PIK3C3; |
| | IRS1; KRAS; SOCS1; PTPN6; |
| | NR3C1; PIK3C2A; JAK1; AKT1; |
| | JAK2; PIK3R1; FRAP1; AKT3; |
| | RPS6KB1 |
| Cell Cycle: G2/M DNA | EP300; PCAF; BRCA1; GADD45A; |
| Damage Checkpoint | PLK1; BTRC; CHEK1; ATR; |
| Regulation | CHEK2; YWHAZ; TP53; CDKN1A; |
| | PRKDC; ATM; SFN; CDKN2A |
| Nitric Oxide | KDR; FLT1; PGF; AKT2; |
| Signaling in the | PIK3CA; PIK3CB; PIK3C3; |
| Cardiovascular | CAV1; PRKCD; NOS3; PIK3C2A; |
| System | |
| | AKT1; PIK3R1; VEGFA; AKT3; |
| | HSP90AA1 |
| Purine Metabolism | NME2; SMARCA4; MYH9; RRM2; |
| | ADAR; EIF2AK4; PKM2; ENTPD1; |
| | RAD51; RRM2B; TJP2; RAD51C; |
| | NT5E; POLD1; NME1 |
| cAMP-mediated | RAP1A; MAPK1; GNAS; CREB1; |
| Signaling | CAMK2A; MAPK3; SRC; RAF1; |
| | MAP2K2; STAT3; MAP2K1; BRAF; |
| | ATF4 |
| Mitochondrial | SOD2; MAPK8; CASP8; MAPK10; |
| Dysfunction | MAPK9; CASP9; PARK7; PSEN1; |
| Notch Signaling | PARK2; APP; CASP3 HES1; |
| | JAG1; NUMB; NOTCH4; ADAM17; |
| | NOTCH2; PSEN1; NOTCH3; |
| | NOTCH1; DLL4 |
| Endoplasmic Reticulum | HSPA5; MAPK8; XBP1; TRAF2; |
| Stress Pathway | ATF6; CASP9; ATF4; EIF2AK3; |
| Pyrimidine Metabolism | CASP3 NME2; AICDA; RRM2; |
| | EIF2AK4; ENTPD1; RRM2B; NT5E; |
| | POLD1; NME1 |
| Parkinson's Signaling | UCHL1; MAPK8; MAPK13; MAPK14; |
| | CASP9; PARK7; PARK2; CASP3 |
| Cardiac & Beta | GNAS; GNAQ; PPP2R1A; GNB2L1; |
| Adrenergic Signaling | PPP2CA; PPP1CC; PPP2R5C |
| Glycolysis/Gluconeogenesis | HK2; GCK; GPI; ALDH1A1; PKM2; |
| | LDHA; HK1 |
| Interferon Signaling | IRF1; SOCS1; JAK1; JAK2; IFITM1; |
| | STAT1; IFIT3 |
| Sonic Hedgehog Signaling | ARRB2; SMO; GLI2; DYRK1A; GLI1; |
| | GSK3B; DYRK1B |
| Glycerophospholipid | PLD1; GRN; GPAM; YWHAZ; SPHK1; |
| Metabolism | SPHK2 |
| Phospholipid Degradation | PRDX6; PLD1; GRN; YWHAZ; SPHK1; |
| | SPHK2 |
| Tryptophan Metabolism | SIAH2; PRMT5; NEDD4; ALDH1A1; |
| | CYP1B1; SIAH1 |
| Lysine Degradation | SUV39H1; EHMT2; NSD1; SETD7; |
| | PPP2R5C |
| Nucleotide Excision | ERCC5; ERCC4; XPA; XPC; ERCC1 |
| Repair Pathway | |
| Starch and Sucrose | UCHL1; HK2; GCK; GPI; HK1 |
| Metabolism | |
| Aminosugars Metabolism | NQO1; HK2; GCK; HK1 |
| Arachidonic Acid | PRDX6; GRN; YWHAZ; CYP1B1 |
| Metabolism | |
| Circadian Rhythm | CSNK1E; CREB1; ATF4; NR1D1 |
| Signaling | |
| Coagulation System | BDKRB1; F2R; SERPINE1; F3 |
| Dopamine Receptor | PPP2R1A; PPP2CA; PPP1CC; PPP2R5C |
| Signaling | |
| Glutathione Metabolism | IDH2; GSTP1; ANPEP; IDH1 |
| Glycerolipid Metabolism | ALDH1A1; GPAM; SPHK1; SPHK2 |
| Linoleic Acid Metabolism | PRDX6; GRN; YWHAZ; CYP1B1 |
| Methionine Metabolism | DNMT1; DNMT3B; AHCY; DNMT3A |
| Pyruvate Metabolism | GLO1; ALDH1A1; PKM2; LDHA |
| Arginine and Proline | ALDH1A1; NOS3; NOS2A |
| Metabolism | |
| Eicosanoid Signaling | PRDX6; GRN; YWHAZ |
| Fructose and Mannose | HK2; GCK; HK1 |
| Metabolism | |
| Galactose Metabolism | HK2; GCK; HK1 |
| Stilbene, Coumarine and | PRDX6; PRDX1; TYR |
| Lignin Biosynthesis | |
| Antigen Presentation | CALR; B2M |
| Pathway | |
| Biosynthesis of Steroids | NQO1; DHCR7 |
| Butanoate Metabolism | ALDH1A1; NLGN1 |
| Citrate Cycle | IDH2; IDH1 |
| Fatty Acid Metabolism | ALDH1A1; CYP1B1 |
| Glycerophospholipid | PRDX6; CHKA |
| Metabolism | |
| Histidine Metabolism | PRMT5; ALDH1A1 |
| Inositol Metabolism | ERO1L; APEX1 |
| Metabolism of Xenobiotics | GSTP1; CYP1B1 |
| by Cytochrome p450 | |
| Methane Metabolism | PRDX6; PRDX1 |
| Phenylalanine Metabolism | PRDX6; PRDX1 |
| Propanoate Metabolism | ALDH1A1; LDHA |
| Selenoamino Acid | PRMT5; AHCY |
| Metabolism | |
| Sphingolipid Metabolism | SPHK1; SPHK2 |
| Aminophosphonate | PRMT5 |
| Metabolism | |
| Androgen and Estrogen | PRMT5 |
| Metabolism | |
| Ascorbate and Aldarate | ALDH1A1 |
| Metabolism | |
| Bile Acid Biosynthesis | ALDH1A1 |
| Cysteine Metabolism | LDHA |
| Fatty Acid Biosynthesis | FASN |
| Glutamate Receptor | GNB2L1 |
| Signaling | |
| NRF2-mediated Oxidative | PRDX1 |
| Stress Response | |
| Pentose Phosphate | GPI |
| Pathway | |
| Pentose and Glucuronate | UCHL1 |
| Interconversions | |
| Retinol Metabolism | ALDH1A1 |
| Riboflavin Metabolism | TYR |
| Tyrosine Metabolism | PRMT5, TYR |
| Ubiquinone Biosynthesis | PRMT5 |
| Valine, Leucine and | ALDH1A1 |
| Isoleucine Degradation | |
| Glycine, Serine and | CHKA |
| Threonine Metabolism | |
| Lysine Degradation | ALDH1A1 |
| Pain/Taste | TRPM5; TRPA1 |
| Pain | TRPM7; TRPC5; TRPC6; TRPC1; |
| | Cnr1; cnr2; Grk2; Trpa1; |
| | Pomc; Cgrp; Crf; Pka; |
| | Era; Nr2b; TRPM5; Prkaca; |
| | Prkacb; Prkar1a; Prkar2a |
| Mitochondrial Function | AIF; CytC; SMAC (Diablo); Aifm-1; |
| | Aifm-2 |
| Developmental Neurology | BMP-4; Chordin (Chrd); Noggin |
| | (Nog); WNT (Wnt2; Wnt2b; Wnt3a; |
| | Wnt4; Wnt5a; Wnt6; Wnt7b; Wnt8b; |
| | Wnt9a; Wnt9b; Wnt10a; Wnt10b; |
| | Wnt16); beta-catenin; Dkk-1; |
| | Frizzled related proteins; Otx-2; |
| | Gbx2; FGF-8; Ree1in; Dab1; unc-86 |
| | (Pou4fl or Brn3a); Numb; Re1n |

Further non-limiting examples of disease-associated genes and polynucleotides and disease specific information that can be treated with the engineered therapeutic polynucleotides of the present invention is available from McKusick-Nathans Institute of Genetic Medicine, Johns Hopkins University (Baltimore, Md.) and National Center for Biotechnology Information, National Library of Medicine (Bethesda, Md.), available on the World Wide Web.

In an aspect, the invention provides a method of individualized or personalized treatment of a genetic disease in a subject in need of such treatment comprising: (a) introducing one or more mutations ex vivo in a tissue, organ or a cell line, or in vivo in a transgenic non-human mammal, comprising delivering to cell(s) of the tissue, organ, cell or mammal a composition comprising the particle delivery system or the delivery system or the virus particle of any one of the above embodiment or the cell of any one of the above embodiment, wherein the specific mutations or precise sequence substitutions are or have been correlated to the genetic disease; (b) testing treatment(s) for the genetic disease on the cells to which the vector has been delivered that have the specific mutations or precise sequence substitutions correlated to the genetic disease; and (c) treating the subject based on results from the testing of treatment(s) of step (b).

In an embodiment, one or more molecules of the engineered delivery system, engineered targeting moieties, polypeptides, viral (e.g., AAV) particles, and/or other particles, polynucleotides, vectors, systems thereof, engineered cells, and/or formulations thereof described herein can be delivered to a subject in need thereof as a therapy for one or more diseases. In an embodiment, the disease to be treated is a genetic or epigenetic based disease. In an embodiment, the disease to be treated is not a genetic or epigenetic based disease. In an embodiment, one or more molecules of the engineered delivery system, engineered targeting moieties, polypeptides, viral (e.g., AAV) particles, and/or other particles, polynucleotides, vectors, and systems thereof, engineered cells, and/or formulations thereof described herein can be delivered to a subject in need thereof as a treatment or prevention (or as a part of a treatment or prevention) of a disease. It will be appreciated that the specific disease to be treated and/or prevented by delivery of an engineered cell and/or engineered can be dependent on the cargo molecule packaged into an engineered AAV capsid particle.

In an embodiment, the engineered therapeutic polynucleotides of the present invention of the present invention can be used in a therapy for treating or preventing a CNS disease, disorder, or a symptom thereof. It will be appreciated that a CNS disease or disorder refers to any disease or disorder whose pathology involves or affects one or more cell types of the central nervous system. In an embodiment, the CNS disease or disorder is one whose primary pathology involves one or more cell types of the CNS. In an embodiment, one or more other cell types outside of the CNS are involved in the pathology of the CNS disease, such as a muscle cell or a peripheral nervous system cell. In an embodiment, the CNS disease or disorder can be caused by one or more genetic abnormalities. In an embodiment, the CNS disease or disorder is not caused by a genetic abnormality. Non-genetic causes of diseases include infection, cancer, physical trauma and others that will be appreciated by those of skill in the art. It also will be apricated that gene modification approaches to treating disease can be applied to treat and/or prevent both genetic diseases and non-genetic diseases. For example, in the case of non-genetic diseases, a gene therapy approach can be used to modify the cause of the non-genetic disease (e.g., a cancer or infectious organism) such that the cause is no longer disease causing (e.g., by eliminating or rendering non-functional the cancer cells or infectious organism).

Exemplary CNS diseases and disorders include, without limitation, Friedreich's Ataxia, Dravet Syndrome, Spinocerebellar Ataxia Type 3, Niemann Pick Type C, Huntington's Disease, Pompe Disease, Myotonic Dystrophy Type 1, Glut1 Deficiency Syndrome (De Vivo Syndrome), Tay-Sachs, Spinal Muscular Atrophy, Alzheimer's disease, Amyotrophic lateral sclerosis (ALS), Danon disease, Rett Syndrome, Angleman Syndrome, infantile neuronal dystorpy, Gaucher's disease, Krabbe disease, metachromatic leukodystrophy, Salla disease, Farber disease or Spinal Musular Atrophy with progressive myoclonic Epilepsy (also reffered to as Jankovic-Rivera syndrome, Unverricht-Lundborg disease, AADC deficiency, Parkinson's disease, Batten disease, a neuronal ceroid lipofuscinosis disease, giant axonal neuropathy, a mucopolysaccharidosis disease (e.g., Hurler syndrome, MPS III A-D), neurofibromatosis, a spinocerebellar ataxia disease, Sandoff disease, GM2 gangliosidosis, Canavan disease, Cockayne syndrome, a pain disease or disorder, a neuropathy or nerve damage, or any combination thereof. Others are described elsewhere herein and/or will be appreciated by those of ordinary skill in the art in view of the description provided herein.

In an embodiment, the compositions described herein can be used for treating or preventing an eye disease or disorder. It will be appreciated that an eye disease or disorder is a disease or disorder that has a pathology or clinical symptom that involves one or more cells or cell types of the eye, including but not limited to, the optic nerve, rods, cones, retinal cells (e.g., photoreceptors, bipolar cells, ganglion cells, horizontal cells, and amacrine cells), and/or the like. The eye disease or disorder can be of genetic or non-genetic origin. Exemplary eye diseases and disoreders include, without limitation, Stargardt disease, a Leber's congenital amaurosis (LCA) (e.g., Leber's congenital amaurosis type 2, LEBER CONGENITALAMAUROSIS (LCA) ANDEARLY-ONSET SEVERE RETINALDYSTROPHY (EOSRD)), Choroideremia, a macular degeneration, diabetic retinopathy, a retinopathy, vitelliform macular dystrophy, a macular dystrophy, Sorsby's fundus dystrophy, cataracts, glaucoma, optic neuropathies, Marfan syndrome, myopia, polypoidal choroidal vasculopathies, retinitis pigmentosa, uveal melanoma, X-linked retinoschisis, pattern dystrophy, achromatopsia, Blue cone monochromatism, Bornholm eye disease, ADGUCAIA-associated COD/CORD, autosomal dominant PRPH2 associated CORD, X-linkedRPGR-associatedCOD/CORD, fundus albipunctatus, Enhanced S-conesyndrome, Bietti crystalline corneoretinaldystorphy, or any combination thereof.

In an embodiment, the compositions described herein can be used for treating or preventing an inner ear disease or disorder. It will be appreciated that an eye disease or disorder is a disease or disorder that has a pathology or clinical symptom that involves one or more cells or cell types of the ear, and more particularly the inner ear, including but not limited to, hair cells, pillar cells, Boettcher's cells, Claudius' cells, spiral ganglion neurons, and Deiters' cells (phalangeal cells). The inner ear disease or disorder can be of genetic or non-genetic origin. Exemplary inner ear disease and disorders include, without limitation, GJB-2 deafness, Jeryell and Lange-Nielsen syndrome, Usher syndrome, Alport syndrome, Branchio-oto-renal syndrome, Waardenburg syndrome, Pendred syndrome, Stickler syndrome, Treacher Collins syndrome, CHARGE syndrome, Norrie disease, Perrault syndrome, Autosomal dominant Nonsyndromic hearing loss, utosomal Recessive Nonsyndromic Hearing Loss, X-linked nonsyndromic hearing loss, an auditory neuropathy, a congenital hearing loss, or any combination thereof.

In an embodiment, the compositions comprising a CNS specific targeting moiety of the present invention and/or cargos that can be delivered by such compositions can be used to treat or prevent pain or a pain disease or disorder in a subject. In an embodiment, a cargo is capable of modulating sensitivity to or pain sensation/perception in a subject. It will be appreciated that depending on the disease or condition, it can be desirable to increase pain sensitivity or perception (e.g., in the case of disease where there is no pain sensitivity) or decrease pain sensitivity, sensation, and/or perception (e.g., neuropathies and others).

In an embodiment, the cargo molecule can treat or prevent a Pain disease or disorder or pain resulting from a disease or disorder. In an embodiment, the pain disease or disorder causes a deleterious insensitivity or lack of sensitivity to pain. In an embodiment, the pain is due to trauma or damage to a tissue and/or nerve(s)/neurons that can be the result of disease (e.g., ischemia, virus, etc.) or external trauma or mechanical pain (e.g., acute injury, surgical wounds and/or amputation, thermal exposure, etc. In an embodiment, the pain disease or disorder involves dysfunction of one or more neurons, ganglions, or other cells of the CNS and/or peripheral nervous system. In an embodiment, the disease or disorder generates inappropriate, hyper-, or other wise deleterious pain negatively impacting quality of life. Exemplary pain diseases or disorders include, without limitation, HSAN-1, HSAN-2, HSAN-3 (familial dysautonomia-pain free phenotype), HSAN-4 (CIPA), mutilated foot, erythermalagia, paroxysmal extreme pain, and other insensitivities to pain, neuropathic pain, other chronic pain, and/or the like. Exemplary targets for genetic modifications for pain modulation include those involved in signal transduction and/or conduction and/or synaptic transmission (TRPV1/2/3/4, P2XR3, TRPM8, TRPA1, P2RX3, P2RY, BDKRB1/2, Htr3A, ACCNs, TRPV4, TRPC/P, ACCN1/2, SCNIOA, SCNIIA, SCN1,3, 4A, SCN9A, KCNQ, (other K+ channel genes), NR1,2, GRIA1-4, GRIC1-5, NKIR, CACNAIA-S, CACNA2D1; genes of the microglia (e.g., TLR2/4. P2RX4/7, CCL2, CX3CRNI), genes of the CNS (e.g., BDNF, OPRDI/K1/M1, CNR1, GABRs, TNF, PLA2), genes of the PNS (e.g., IL1/6/12/18, COX-2, NTRK1, NGF, GDNF, TNF, LIF, CCL2, CNR2), genes and/or any one or more of the SNPs set forth in Table 1 of Foulkes and Wood. PLOS Genetics. 2008. doi.org/10.1371/journal.pgen. 1000086; any one or more genes associated with a heritable pain condition (e.g., SPTLC1, IkbKAP protein gene, CCT4, Nav1.7 gene); ion channel related genes (e.g., (SCN9A, CACNG2, ZSCAN20, SCN11A), Neurotransmission (OPRM1, COMT, PRKCA, SLCA4, MPZ, GCH1), Metabolism (GCH1, TF, CP, TFRC, ACO1, FXN, SLC11A2, B2M, BMP6), Immune Response (HLA-A, HLA-B, HLA-DQB1, HLA-DRB1, IL6, ILIR2, IL10, TNF-α, GFRA2, HMGB1P46), SCN9A (NaV1.7), SCN10A (NaV1.8) and SCN11A (NaV1.9), GAD, or any combination thereof. In an embodiment, the cargo is a glutamic acid decarboxylase (GAD) which can provide GABA to recue pain, such as neuropathic pain. In an embodiment, the pain-associated genes are modified using a CRISPRi approach (e.g., the engineered therapeutic polynucleotides of the present invention can contain CRISPRi molecule(s). In an embodiment, the pain-associated genes are modified using a CRISPRi-KRAB approach. See also e.g., Wolfe et al., Pain Medicine, Volume 10, Issue 7, October 2009, Pages 1325-1330, Moreno A M, Glaucilene F C, Alemán F et al. Long-lasting analgesia via targeted in vivoepigenetic repression of Nav1.7. bioRxiv711812 (2019). biorxiv.org/content/10.1101/71, Foulkes and Wood. PLOS Genetics. 2008. doi.org/10.1371/journal.pgen.1000086, the teachings of which can be adapted for use with the present invention.

Genetic diseases that can be treated are discussed in greater detail elsewhere herein. Other diseases that can be treated by the compositions of the present invention can include, but are not limited to, any of the following: cancer (such as glioblastoma or other brain or CNS cancers), Acubetivacter infections, actinomycosis, African sleeping sickness, AIDS/HIV, ameobiasis, Anaplasmosis, Angiostrongyliasis, Anisakiasis, Anthrax, Acranobacterium haemolyticum infection, Argentine hemorrhagic fever, Ascariasis, Aspergillosis, Astrovirus infection, Babesiosis, Bacterial meningitis, Bacterial pneumonia, Bacterial vaginosis, Bacteroides infection, balantidiasis, Bartonellosis, Baylisascaris infection, BK virus infection, Black Piedra, Blastocytosis, Blastomycosis, Bolivian hemorrhagic fever, Botulism, Brazillian hemmorhagic fever, brucellosis, Bubonic plague, Burkholderia infection, buruli ulcer, calicivirus invention, campylobacteriosis, Candidasis, Capillariasis, Carrion's disease, Cat-scratch disease, cellulitis, Chagas Disease, Chancroid, Chickenpox, Chikungunya, Chlamydia, Chlamydia pneumoniae, Cholera, Chromoblastomycosis, Chytridiomycosis, Clonochiasis, Clostridium difficile colitis, Coccidioidomycosis, Colorado tick fever, rhinovirus/coronavirus invection (common cold), Cretzfeldt-Jakob disease, Crimean-congo hemorrhagic fever, Cryptococcosis, Cryptosporidosis, Cutaneous larva migrans (CLM), cyclosporiasis, cysticercosis, cytomegalovirus infection, Dengue fever, Desmodesmus infection, Dientamoebiasis, Diptheria, Diphylobothriasis, Dracunculiasis, Ebola, Echinococcosis, Ehrlichiosis, Enterobiasis, Enterococcus infection, Enterovirus infection, Epidemic typhus, Erthemia Infectisoum, Exanthem subitum, Fasciolasis, Fasciolopsiasis, fatal familial insomnia, filarisis, Clostridum perfingens infection, Fusobacterium infection, Gas gangrene (clostridial myonecrosis), Geotrichosis, Gerstmann-Straussler-Scheinker syndrome, Giardasis, Glanders, Gnathostomiasis, Gonorrhea, Granuloma inguinales, Group A streptococcal infection, Group B streptococcal infection, Haemophilus influenzae infection, Hand, foot, and mouth disease, hanta virus pulmonary syndrome, heartland virus disease, Helicobacter pylori infection, hemorrhagi fever with renal syndrome, Hendra virus infection, Hepatitis (all groups A, B, C, D, E), hepes simplex, histoplasmosis, hookworm infection, human bocavirus infection, human ewingii erlichosis, Human granulocytic anaplasmosis, human metapneymovirus infection, human monocytic ehrlichosis, human papaloma virus, Hymenolepiasis, Epstein-Barr infection, mononucleosis, influenza, isoporisis, Kawasaki disease, Kingell kingae infection, Kuru, Lasas fever, Leginollosis (Legionnaires's disease and Potomac Fever), Leishmaniasis, Leprosy, Leptospirosis, Listeriosis, Lyme disease, lymphatic filariasis, lymphocytic choriomeningitis, Malaria, Marburg hemorrhagic feaver, measals, Middle East respiratory syndrome, Meliodosis, menigitis, Menigococcal disease, Metagonimiasis, Microsporidosis, Molluscum contagiosum, Monkeypox, Mumps, Murine typhus, Mycoplasma pneumonia, Mycoplasma genitalium infection, Mycetoma, Myiasis, Conjunctivitis, Nipah virus infection, Norovirus, Variant Creutzfeldt-Jakob disease, Nocardosis, Onchocerciasis, Opisthorchiasis, Paracoccidioidomycosis, Paragonimiasis, Pasteurellosis, Pdiculosisi capitis, Pediculosis corpis, Pediculosis pubis, pelvic inflammatory disease, pertussis, plague, pneumococcal infection, pneumocystis pneumonia, pneumonia, poliomyelitis, prevotella infection, primary amoebic menigoencephalitis, progressive multifocal leukoencephalopathy, Psittacosis, Qfever, rabies, relapsing fever, respiratory syncytial virus infection, rhinovirus infection, rickettsial infection, Rickettsialpox, Rift Valley Fever, Rocky Mountain Spotted Fever, Rotavirus infection, Rubella, Salmonellosis, SARS, Scabies, Scarlet fever, Schistosomiais, Sepsis, Shigellosis, Shingles, Smallpox, Sporotrichosisi, Staphlococcol infection (including MRSA), strongyloidiasis, subacute sclerosing panecephalitis, Syphillis, Taeniasis, tetanus, Trichophyton species infection, Tocariasis, Toxoplasmosis, Trachoma, Trichinosis, Trichuriasis, Tuberculosis, Tularemia, Typhoid Fever, Typhus Fever, Ureaplasma urealyticum infection, Valley fever, Venezuelan equine encephalitis, Venezuelan hemorrhagic fever, Vibrio species infection, Viral pneumonia, West Nile Fever, White Piedra, Yersinia pseudotuberculosis, Yersiniosis, Yellow fever, Zeaspora, Zika fever, Zygomycosis and combinations thereof.

Other diseases and disorders or symptoms thereof that can be treated using embodiments of the present invention include, but are not limited to, endocrine diseases (e.g., Type I and Type II diabetes, gestational diabetes, hypoglycemia. Glucagonoma, Goitre, Hyperthyroidism, hypothyroidism, thyroiditis, thyroid cancer, thyroid hormone resistance, parathyroid gland disorders, Osteoporosis, osteitis deformans, rickets, ostomalacia, hypopituitarism, pituitary tumors, etc.), skin conditions of infections and non-infection origin, eye diseases of infectious or non-infectious origin, gastrointestinal disorders of infectious or non-infectious origin, cardiovascular diseases of infectious or non-infectious origin, brain and neuron diseases of infectious or non-infectious origin, nervous system diseases of infectious or non-infectious origin, muscle diseases of infectious or non-infectious origin, bone diseases of infectious or non-infectious origin, reproductive system diseases of infectious or non-infectious origin, renal system diseases of infectious or non-infectious origin, blood diseases of infectious or non-infectious origin, lymphatic system diseases of infectious or non-infectious origin, immune system diseases of infectious or non-infectious origin, mental-illness of infectious or non-infectious origin and the like.

In an embodiment, the disease to be treated is a CNS or CNS related disease or disorder, such as a genetic CNS disease or disorder. Such CNS or CNS related disease (including genetic CNS disease or disorders) are described in greater detail elsewhere herein. Other diseases and disorders will be appreciated by those of skill in the art.

### Infectious Diseases

In an embodiment, the compositions of the present invention thereof can be used to diagnose, prognose, treat, and/or prevent an infectious disease caused by a microorganism, such as bacteria, virus, fungi, parasites, or combinations thereof.

In an embodiment, the engineered therapeutic polynucleotides of the present invention can be capable of targeting pathogenic and/or drug-resistant microorganisms, such as bacteria, virus, parasites, and fungi. In an embodiment, the engineered therapeutic polynucleotides of the present invention can be capable of targeting and modifying one or more polynucleotides in a pathogenic microorganism such that the microorganism is less virulent, killed, inhibited, or is otherwise rendered incapable of causing disease and/or infecting and/or replicating in a host cell.

In an embodiment, the pathogenic bacteria that can be targeted and/or modified by the engineered therapeutic polynucleotides of the present invention described herein include, but are not limited to, those of the genus Actinomyces (e.g. A. israelii), Bacillus (e.g. B. anthracis, B. cereus), Bactereoides (e.g. B. fragilis), Bartonella (B. henselae, B. quintana), Bordetella (B. pertussis), Borrelia (e.g. B. burgdorferi, B. garinii, B. afzelii, and B. recurreentis), Brucella (e.g. B. abortus, B. canis, B. melitensis, and B. suis), Campylobacter (e.g. C. jejuni), Chlamydia (e.g. C. pneumoniae and C. trachomatis), Chlamydophila (e.g. C. psittaci), Clostridium (e.g. C. botulinum, C. difficile, C. perfringens. C. tetani), Corynebacterium (e.g. C. diptheriae), Enterococcus (e.g. E. Faecalis, E. faecium), Ehrlichia (E. canis and E. chaffensis) Escherichia (e.g. E. coli), Francisella (e.g. F. tularensis), Haemophilus (e.g. H. influenzae), Helicobacter (H. pylori), Klebsiella (E.g. K. pneumoniae), Legionella (e.g. L. pneumophila), Leptospira (e.g. L. interrogans, L. santarosai, L. weilii, L. noguchii), Listereia (e.g. L. monocytogeenes), Mycobacterium (e.g. M. leprae, M. tuberculosis, M. ulcerans), Mycoplasma (M. pneumoniae), Neisseria (N. gonorrhoeae and N. menigitidis), Nocardia (e.g. N. asteeroides), Pseudomonas (P. aeruginosa), Rickettsia (R. rickettsia), Salmonella (S. typhi and S. typhimurium), Shigella (S. sonnei and S. dysenteriae), Staphylococcus (S. aureus, S. epidermidis, and S. saprophyticus), Streeptococcus (S. agalactiaee, S. pneumoniae, S. pyogenes), Treponema (T. pallidum), Ureeaplasma (e.g. U. urealyticum), Vibrio (e.g. V. cholerae), Yersinia (e.g. Y. pestis, Y. enteerocolitica, and Y. pseudotuberculosis).

In an embodiment, the pathogenic virus that can be targeted and/or modified by the CRISPR-Cas system(s) and/or component(s) thereof described herein include, but are not limited to, a double-stranded DNA virus, a partly double-stranded DNA virus, a single-stranded DNA virus, a positive single-stranded RNA virus, a negative single-stranded RNA virus, or a double stranded RNA virus. In an embodiment, the pathogenic virus can be from the family Adenoviridae (e.g. Adenovirus), Herpeesviridae (e.g. Herpes simplex, type 1, Herpes simplex, type 2, Varicella-zoster virus, Epstein-Barr virus, Human cytomegalovirus, Human herpesvirus, type 8), Papillomaviridae (e.g. Human papillomavirus), Polyomaviridae (e.g. BK virus, JC virus), Poxviridae (e.g. smallpox), Hepadnaviridae (e.g. Hepatitis B), Parvoviridae (e.g. Parvovirus B19), Astroviridae (e.g. Human astrovirus), Caliciviridae (e.g. Norwalk virus), Picornaviridae (e.g. coxsackievirus, hepatitis A virus, poliovirus, rhinovirus), Coronaviridae (e.g. Severe acute respiratory syndrome-related coronavirus, strains: Severe acute respiratory syndrome virus, Severe acute respiratory syndrome coronavirus 2 (COVID-19)), Flaviviridae (e.g. Hepatitis C virus, yellow fever virus, dengue virus, West Nile virus, TBE virus), Togaviridae (e.g. Rubella virus), Hepeviridae (e.g. Hepatitis E virus), Retroviridae (Human immunodeficiency virus (HIV)), Orthomyxoviridae (e.g. Influenza virus), Arenaviridae (e.g. Lassa virus), Bunyaviridae (e.g. Crimean-Congo hemorrhagic fever virus, Hantaan virus), Filoviridae (e.g. Ebola virus and Marburg virus), Paramyxoviridae (e.g. Measles virus, Mumps virus, Parainfluenza virus, Respiratory syncytial virus), Rhabdoviridae (Rabies virus), Hepatits D virus, Reoviridae (e.g. Rotavirus, Orbivirus, Coltivirus, Banna virus).

In an embodiment, the pathogenic fungi that can be targeted and/or modified by the CRISPR-Cas system(s) and/or component(s) thereof described herein include, but are not limited to, those of the genus Candida (e.g. C. albicans), Aspergillus (e.g. A. fumigatus, A. flavus, A. clavatus), Cryptococcus (e.g. C. neoformans, C. gattii), Histoplasma (H. capsulatum), Pneumocystis (e.g. P. jiroveecii), Stachybotrys (e.g. S. chartarum).

In an embodiment, the pathogenic parasites that can be targeted and/or modified by the engineered therapeutic polynucleotides of the present invention include, but are not limited to, protozoa, helminths, and ectoparasites. In an embodiment, the pathogenic protozoa that can be targeted and/or modified by the engineered therapeutic polynucleotides of the present invention include, but are not limited to, those from the groups Sarcodina (e.g. ameba such as Entamoeba), Mastigophora (e.g. flagellates such as Giardia and Leishmania), Cilophora (e.g. ciliates such as Balantidum), and sporozoa (e.g. plasmodium and cryptosporidium). In an embodiment, the pathogenic helminths that can be targeted and/or modified by the engineered therapeutic polynucleotides of the present invention include, but are not limited to, flatworms (platyhelminths), thorny-headed worms (acanthoceephalins), and roundworms (nematodes). In an embodiment, the pathogenic ectoparasites that can be targeted and/or modified by the engineered therapeutic polynucleotides of the present invention include, but are not limited to, ticks, fleas, lice, and mites.

In an embodiment, the pathogenic parasite that can be targeted and/or modified by the engineered therapeutic polynucleotides of the present invention include, but are not limited to, Acanthamoeba spp., Balamuthia mandrillaris, Babesiosis spp. (e.g. Babesia B. divergens, B. bigemina, B. equi, B. microfti, B. duncani), Balantidiasis spp. (e.g. Balantidium coli), Blastocystis spp., Cryptosporidium spp., Cyclosporiasis spp. (e.g. Cyclospora cayetanensis), Dientamoebiasis spp. (e.g. Dientamoeba fragilis), Amoebiasis spp. (e.g. Entamoeba histolytica), Giardiasis spp. (e.g. Giardia lamblia), Isosporiasis spp. (e.g. Isospora belli), Leishmania spp., Naegleria spp. (e.g. Naegleria fowleri), Plasmodium spp. (e.g. Plasmodium falciparum, Plasmodium vivax, Plasmodium ovale curtisi, Plasmodium ovale wallikeri, Plasmodium malariae, Plasmodium knowlesi), Rhinosporidiosis spp. (e.g. Rhinosporidium seeberi), Sarcocystosis spp. (e.g. Sarcocystis bovihominis, Sarcocystis suihominis), Toxoplasma spp. (e.g. Toxoplasma gondii), Trichomonas spp. (e.g. Trichomonas vaginalis), Trypanosoma spp. (e.g. Trypanosoma brucei), Trypanosoma spp. (e.g. Trypanosoma cruzi), Tapeworm (e.g. Cestoda, Taenia multiceps, Taenia saginata, Taenia solium), Diphyllobothrium latum spp., Echinococcus spp. (e.g. Echinococcus granulosus, Echinococcus multilocularis, E. vogeli, E. oligarthrus), Hymenolepis spp. (e.g. Hymenolepis nana, Hymenolepis diminuta), Bertiella spp. (e.g. Bertiella mucronata, Bertiella studeri), Spirometra (e.g. Spirometra erinaceieuropaei), Clonorchis spp. (e.g. Clonorchis sinensis; Clonorchis viverrini), Dicrocoelium spp. (e.g. Dicrocoelium dendriticum), Fasciola spp. (e.g. Fasciola hepatica, Fasciola gigantica), Fasciolopsis spp. (e.g. Fasciolopsis buski), Metagonimus spp. (e.g. Metagonimus yokogawai), Metorchis spp. (e.g. Metorchis conjunctus), Opisthorchis spp. (e.g. Opisthorchis viverrini, Opisthorchis felineus), Clonorchis spp. (e.g. Clonorchis sinensis), Paragonimus spp. (e.g. Paragonimus westermani; Paragonimus africanus; Paragonimus caliensis; Paragonimus kellicotti; Paragonimus skrjabini; Paragonimus uterobilateralis), Schistosoma sp., Schistosoma spp. (e.g. Schistosoma mansoni, Schistosoma haematobium, Schistosoma japonicum, Schistosoma mekongi, and Schistosoma intercalatum), Echinostoma spp. (e.g. E. echinatum), Trichobilharzia spp. (e.g. Trichobilharzia regent), Ancylostoma spp. (e.g. Ancylostoma duodenale), Necator spp. (e.g. Necator americanus), Angiostrongylus spp., Anisakis spp., Ascaris spp. (e.g. Ascaris lumbricoides), Baylisascaris spp. (e.g. Baylisascaris procyonis), Brugia spp. (e.g. Brugia malayi, Brugia timori), Dioctophyme spp. (e.g. Dioctophyme renale), Dracunculus spp. (e.g. Dracunculus medinensis), Enterobius spp. (e.g. Enterobius vermicularis, Enterobius gregorii), Gnathostoma spp. (e.g. Gnathostoma spinigerum, Gnathostoma hispidum), Halicephalobus spp. (e.g. Halicephalobus gingivalis), Loa loa spp. (e.g. Loa loa filaria), Mansonella spp. (e.g. Mansonella streptocerca), Onchocerca spp. (e.g. Onchocerca volvulus), Strongyloides spp. (e.g. Strongyloides stercoralis), Thelazia spp. (e.g. Thelazia californiensis, Thelazia callipaeda), Toxocara spp. (e.g. Toxocara canis, Toxocara cati, Toxascaris leonine), Trichinella spp. (e.g. Trichinella spiralis, Trichinella britovi, Trichinella nelsoni, Trichinella nativa), Trichuris spp. (e.g. Trichuris trichiura, Trichuris vulpis), Wuchereria spp. (e.g. Wuchereria bancrofti), Dermatobia spp. (e.g. Dermatobia hominis), Tunga spp. (e.g. Tunga penetrans), Cochliomyia spp. (e.g. Cochliomyia hominivorax), Linguatula spp. (e.g. Linguatula serrata), Archiacanthocephala sp., Moniliformis sp. (e.g. Moniliformis moniliformis), Pediculus spp. (e.g. Pediculus humanus capitis, Pediculus humanus humanus), Pthirus spp. (e.g. Pthirus pubis), Arachnida spp. (e.g. Trombiculidae, Ixodidae, Argaside), Siphonaptera spp (e.g. Siphonaptera: Pulicinae), Cimicidae spp. (e.g. Cimex lectularius and Cimex hemipterus), Diptera spp., Demodex spp. (e.g. Demodex folliculorum/brevis/canis), Sarcoptes spp. (e.g. Sarcoptes scabiei), Dermanyssus spp. (e.g. Dermanyssus gallinae), Ornithonyssus spp. (e.g. Ornithonyssus sylviarum, Ornithonyssus bursa, Ornithonyssus bacoti), Laelaps spp. (e.g. Laelaps echidnina), Liponyssoides spp. (e.g. Liponyssoides sanguineus).

In an embodiment the gene targets can be any of those as set forth in Table 1 of Strich and Chertow. 2019. J. Clin. Microbio. 57:4 e01307-18, which is incorporated herein as if expressed in its entirety herein.

In an embodiment, the method can include delivering and/or expressing the engineered therapeutic polynucleotides of the present invention to a pathogenic organism described herein, allowing the engineered therapeutic polynucleotides of the present invention modify one or more targets in the pathogenic organism, whereby the modification kills, inhibits, reduces the pathogenicity of the pathogenic organism, or otherwise renders the pathogenic organism non-pathogenic. In an embodiment, delivery occurs in vivo (i.e., in the subject being treated). In an embodiment occurs by an intermediary, such as microorganism or phage that is non-pathogenic to the subject but is capable of transferring polynucleotides and/or infecting the pathogenic microorganism. In an embodiment, the intermediary microorganism can be an engineered bacteria, virus, or phage that contains the composition of the present invention. The method can include administering an intermediary microorganism containing the composition of the present invention to the subject to be treated. The intermediary microorganism can then produce a therapeutic polynucleotide or gene product therefrom or transfer a therapeutic polynucleotide or gene product therefrom to the pathogenic organism. In embodiments, where the therapeutic polynucleotide or gene product therefrom is transferred to the pathogenic microorganism, the genetic modification system or component thereof is then produced in the pathogenic microorganism and modifies the pathogenic microorganism such that it is less virulent, killed, inhibited, or is otherwise rendered incapable of causing disease and/or infecting and/or replicating in a host or cell thereof.

In an embodiment, where the pathogenic microorganism inserts its genetic material into the host cell's genome (e.g. a virus), the engineered therapeutic polynucleotide can be designed such that it modifies the host cell's genome such that the viral DNA or cDNA cannot be replicated by the host cell's machinery into a functional virus. In an embodiment, where the pathogenic microorganism inserts its genetic material into the host cell's genome (e.g. a virus), the CRISPR-Cas system can be designed such that it modifies the host cell's genome such that the viral DNA or cDNA is deleted from the host cell's genome.

It will be appreciated that inhibiting or killing the pathogenic microorganism, the disease and/or condition that its infection causes in the subject can be treated or prevented. Thus, also provided herein are methods of treating and/or preventing one or more diseases or symptoms thereof caused by any one or more pathogenic microorganisms, such as any of those described herein.

### Example Microbes

In an embodiment, the engineered polynucleotides of the present intention disclosed herein may be used to detect and/or kill a number of different microbes. The term microbe as used herein includes bacteria, fungus, protozoa, parasites and viruses. Exemplary microbes are now described.

### Bacteria

The following provides an example list of the types of microbes that might be detected using the embodiments disclosed herein. In certain example embodiments, the microbe is a bacterium. Examples of bacteria that can be detected in accordance with the disclosed methods include without limitation any one or more of (or any combination of) Acinetobacter baumanii, Actinobacillus sp., Actinomycetes, Actinomyces sp. (such as Actinomyces israelii and Actinomyces naeslundii), Aeromonas sp. (such as Aeromonas hydrophila, Aeromonas veronii biovar sobria (Aeromonas sobria), and Aeromonas caviae), Anaplasma phagocytophilum, Anaplasma marginale Alcaligenes xylosoxidans, Acinetobacter baumanii, Actinobacillus actinomycetemcomitans, Bacillus sp. (such as Bacillus anthracis, Bacillus cereus, Bacillus subtilis, Bacillus thuringiensis, and Bacillus stearothermophilus), Bacteroides sp. (such as Bacteroides fragilis), Bartonella sp. (such as Bartonella bacilliformis and Bartonella henselae, Bifidobacterium sp., Bordetella sp. (such as Bordetella pertussis, Bordetella parapertussis, and Bordetella bronchiseptica), Borrelia sp. (such as Borrelia recurrentis, and Borrelia burgdorferi), Brucella sp. (such as Brucella abortus, Brucella canis, Brucella melintensis and Brucella suis), Burkholderia sp. (such as Burkholderia pseudomallei and Burkholderia cepacia), Campylobacter sp. (such as Campylobacter jejuni, Campylobacter coli, Campylobacter lari and Campylobacter fetus), Capnocytophaga sp., Cardiobacterium hominis, Chlamydia trachomatis, Chlamydophila pneumoniae, Chlamydophila psittaci, Citrobacter sp. Coxiella burnetii, Corynebacterium sp. (such as, Corynebacterium diphtheriae, Corynebacterium jeikeum and (orynebacterium), Clostridium sp. (such as Clostridium perfringens, Clostridium difficile, Clostridium botulinum and Clostridium tetani), Eikenella corrodens, Enterobacter sp. (such as Enterobacter aerogenes, Enterobacter agglomerans, Enterobacter cloacae and Escherichia coli, including opportunistic Escherichia coli, such as enterotoxigenic E. coli, enteroinvasive E. coli, enteropathogenic E. coli, enterohemorrhagic E. coli, enteroaggregative E. coli and uropathogenic E. coli) Enterococcus sp. (such as Enterococcus faecalis and Enterococcus faecium) Ehrlichia sp. (such as Ehrlichia chafeensia and Ehrlichia canis), Epidermophyton floccosum, Erysipelothrix rhusiopathiae, Eubacterium sp., Francisella tularensis, Fusobacterium nucleatum, Gardnerella vaginalis, Gemella morbillorum, Haemophilus sp. (such as Haemophilus influenzae, Haemophilus ducreyi, Haemophilus aegyptius, Haemophilus parainfluenzae, Haemophilus haemolyticus and Haemophilus parahaemolyticus, Helicobacter sp. (such as Helicobacter pylori, Helicobacter cinaedi and Helicobacter fennelliae), Kingella kingii, Klebsiella sp. (such as Klebsiella pneumoniae, Klebsiella granulomatis and Klebsiella oxytoca), Lactobacillus sp., Listeria monocytogenes, Leptospira interrogans, Legionella pneumophila, Leptospira interrogans, Peptostreptococcus sp., Mannheimia hemolytica, Microsporum canis, Moraxella catarrhalis, Morganella sp., Mobiluncus sp., Micrococcus sp., Mycobacterium sp. (such as Mycobacterium leprae, Mycobacterium tuberculosis, Mycobacterium paratuberculosis, Mycobacterium intracellulare, Mycobacterium avium, Mycobacterium bovis, and Mycobacterium marinum), Mycoplasm sp. (such as Mycoplasma pneumoniae, Mycoplasma hominis, and Mycoplasma genitalium), Nocardia sp. (such as Nocardia asteroides, Nocardia cyriacigeorgica and Nocardia brasiliensis), Neisseria sp. (such as Neisseria gonorrhoeae and Neisseria meningitidis), Pasteurella multocida, Pityrosporum orbiculare (Malassezia furfur), Plesiomonas shigelloides. Prevotella sp., Porphyromonas sp., Prevotella melaninogenica, Proteus sp. (such as Proteus vulgaris and Proteus mirabilis), Providencia sp. (such as Providencia alcalifaciens, Providencia rettgeri and Providencia stuartii), Pseudomonas aeruginosa, Propionibacterium acnes, Rhodococcus equi, Rickettsia sp. (such as Rickettsia rickettsii, Rickettsia akari and Rickettsia prowazekii, Orientia tsutsugamushi (formerly: Rickettsia tsutsugamushi) and Rickettsia typhi), Rhodococcus sp., Serratia marcescens, Stenotrophomonas maltophilia, Salmonella sp. (such as Salmonella enterica, Salmonella typhi, Salmonella paratyphi, Salmonella enteritidis, Salmonella cholerasuis and Salmonella typhimurium), Serratia sp. (such as Serratia marcesans and Serratia liquifaciens), Shigella sp. (such as Shigella dysenteriae, Shigella flexneri, Shigella boydii and Shigella sonnei), Staphylococcus sp. (such as Staphylococcus aureus, Staphylococcus epidermidis, Staphylococcus hemolyticus, Staphylococcus saprophyticus), Streptococcus sp. (such as Streptococcus pneumoniae (for example chloramphenicol-resistant serotype 4 Streptococcus pneumoniae, spectinomycin-resistant serotype 6B Streptococcus pneumoniae, streptomycin-resistant serotype 9V Streptococcus pneumoniae, erythromycin-resistant serotype 14 Streptococcus pneumoniae, optochin-resistant serotype 14 Streptococcus pneumoniae, rifampicin-resistant serotype 18C Streptococcus pneumoniae, tetracycline-resistant serotype 19F Streptococcus pneumoniae, penicillin-resistant serotype 19F Streptococcus pneumoniae, and trimethoprim-resistant serotype 23F Streptococcus pneumoniae, chloramphenicol-resistant serotype 4 Streptococcus pneumoniae, spectinomycin-resistant serotype 6B Streptococcus pneumoniae, streptomycin-resistant serotype 9V Streptococcus pneumoniae, optochin-resistant serotype 14 Streptococcus pneumoniae, rifampicin-resistant serotype 18C Streptococcus pneumoniae, penicillin-resistant serotype 19F Streptococcus pneumoniae, or trimethoprim-resistant serotype 23F Streptococcus pneumoniae), Streptococcus agalactiae, Streptococcus mutans, Streptococcus pyogenes, Group A streptococci, Streptococcus pyogenes, Group B streptococci, Streptococcus agalactiae, Group (streptococci, Streptococcus anginosus, Streptococcus equismilis, Group D) streptococci, Streptococcus bovis, Group F streptococci, and Streptococcus anginosus Group G streptococci), Spirillum minus, Streptobacillus moniliformi, Treponema sp. (such as Treponema carateum, Treponema petemie, Treponema pallidum and Treponema endemicum, Trichophyton rubrum, T. mentagrophytes, Tropheryma whippelii, Ureaplasma urealyticum, Veillonella sp., Vibrio sp. (such as Vibrio cholerae, Vibrio parahemolyticus, Vibrio vulnificus, Vibrio parahaemolyticus, Vibrio vulnificus, Vibrio alginolyticus, Vibrio mimicus, Vibrio hollisae, Vibrio fluvialis, Vibrio metchnikovii, Vibrio damsela and Vibrio furnisii), Yersinia sp. (such as Yersinia enterocolitica, Yersinia pestis, and Yersinia pseudotuberculosis) and Xanthomonas maltophilia among others.

Near-real-time microbial diagnostics are needed for food, clinical, industrial, and other environmental settings (see e.g., Lu T K, Bowers J, and Koeris M S., Trends Biotechnol. 2013 June; 31 (6): 325-7). In certain embodiments, the assay described herein is configured for detection of foodborne pathogens using guide RNAs specific to a pathogen (e.g., Campylobacter jejuni, Clostridium perfringens, Salmonella spp., Escherichia coli, Bacillus cereus, Listeria monocytogenes, Shigella spp., Staphylococcus aureus, Staphylococcal enteritis, Streptococcus, Vibrio cholerae, Vibrio parahaemolyticus, Vibrio vulnificus, Yersinia enterocolitica and Yersinia pseudotuberculosis, Brucella spp., Corynebacterium ulcerans, Coxiella burnetii, or Plesiomonas shigelloides).

### Fungi

In certain example embodiments, the microbe is a fungus or a fungal species. Examples of fungi that can be detected in accordance with the disclosed methods include without limitation any one or more of (or any combination of), Aspergillus, Blastomyces, Candidiasis, Coccidiodomycosis, Cryptococcus neoformans, Cryptococcus gatti, sp. Histoplasma sp. (such as Histoplasma capsulatum), Pneumocystis sp. (such as Pneumocystis jirovecii), Stachybotrys (such as Stachybotrys chartarum), Mucroymcosis, Sporothrix, fungal eye infections ringworm, Exserohilum, Cladosporium.

In certain example embodiments, the fungus is a yeast. Examples of yeast that can be detected in accordance with disclosed methods include without limitation one or more of (or any combination of), Aspergillus species (such as Aspergillus fumigatus, Aspergillus flavus and Aspergillus clavatus), Cryptococcus sp. (such as Cryptococcus neoformans, Cryptococcus gattii, Cryptococcus laurentii and Cryptococcus albidus), a Geotrichum species, a Saccharomyces species, a Hansemila species, a Candida species (such as Candida albicans), a Kluyveromyces species, a Debaryomyces species, a Pichia species, or combination thereof. In certain example embodiments, the fungi is a mold. Example molds include, but are not limited to, a Penicillium species, a Cladosporium species, a Byssochlamys species, or a combination thereof.

### Protozoa

In certain example embodiments, the microbe is a protozoan. Examples of protozoa that can be detected in accordance with the disclosed methods and devices include without limitation any one or more of (or any combination of), Euglenozoa, Heterolobosea, Diplomonadida, Amoebozoa, Blastocystic, and Apicomplexa. Example Euglenoza include, but are not limited to, Trypanosoma cruzi (Chagas disease), T. brucei gambiense, T. brucei rhodesiense, Leishmania braziliensis, L. infantum, L. mexicana, L. major, L. tropica, and L. donovani. Example Heterolobosea include, but are not limited to, Naegleria fowleri. Example Diplomonadid include, but are not limited to, Giardia intestinalis (G. lamblia, G. duodenalis). Example Amoebozoa include, but are not limited to, Acanthamoeba castellanii, Balamuthia madrillaris, Entamoeba histolytica. Example Blastocystis include, but are not limited to, Blastocystic hominis. Example Apicomplexa include, but are not limited to, Babesia microti, Cryptosporidium parvum, Cyclospora cayetanensis, Plasmodium falciparum, P. vivax, P. ovale, P. malariae, and Toxoplasma gondii.Babesia microti, Cryptosporidium parvum, Cyclospora cayetanensis, Plasmodium falciparum, P. vivax, P. ovale, P. malariae, and Toxoplasma gondii.

### Parasites

In certain example embodiments, the microbe is a parasite. Examples of parasites that can be detected in accordance with disclosed methods include without limitation one or more of (or any combination of), an Onchocerca species and a Plasmodium species.

### Viruses

In certain example embodiments, the systems, devices, and methods, disclosed herein are directed to detecting viruses in a sample. The embodiments disclosed herein may be used to detect viral infection (e.g. of a subject or plant), or determination of a viral strain, including viral strains that differ by a single nucleotide polymorphism. The virus may be a DNA virus, a RNA virus, or a retrovirus. Non-limiting example of viruses useful with the present invention include, but are not limited to Ebola, measles, SARS, Chikungunya, hepatitis, Marburg, yellow fever, MERS, Dengue, Lassa, influenza, rhabdovirus or HIV. A hepatitis virus may include hepatitis A, hepatitis B, or hepatitis C. An influenza virus may include, for example, influenza A or influenza B. An HIV may include HIV 1 or HIV 2. In certain example embodiments, the viral sequence may be a human respiratory syncytial virus, Sudan ebola virus, Bundibugyo virus, Tai Forest ebola virus, Reston ebola virus, Achimota, Aedes flavivirus, Aguacate virus, Akabane virus, Alethinophid reptarenavirus, Allpahuayo mammarenavirus, Amapari mmarenavirus, Andes virus, Apoi virus, Aravan virus, Aroa virus, Arumwot virus, Atlantic salmon paramyoxivirus, Australian bat lyssavirus, Avian bornavirus, Avian metapneumovirus, Avian paramyoxviruses, penguin or Falkland Islandsvirus, BK polyomavirus, Bagaza virus, Banna virus, Bat hepevirus, Bat sapovirus, Bear Canon mammarenavirus, Beilong virus, Betacoronoavirus, Betapapillomavirus 1-6, Bhanja virus, Bokeloh bat lyssavirus, Borna disease virus, Bourbon virus, Bovine hepacivirus, Bovine parainfluenza virus 3, Bovine respiratory syncytial virus, Brazoran virus, Bunyamwere virus, Caliciviridae virus. California encephalitis virus, Candiru virus, Canine distemper virus, Canaine pneumovirus, Cedar virus, Cell fusing agent virus, Cetacean morbillivirus, Chandipura virus, Chaoyang virus, Chapare mammarenavirus, Chikungunya virus, Colobus monkey papillomavirus, Colorado tick fever virus, Cowpox virus, Crimean-Congo hemorrhagic fever virus, Culex flavivirus, Cupixi mammarenavirus, Dengue virus, Dobrava-Belgrade virus, Donggang virus, Dugbe virus, Duvenhage virus, Eastern equine encephalitis virus, Entebbe bat virus, Enterovirus A-D, European bat lyssavirus 1-2, Eyach virus, Feline morbillivirus, Fer-de-Lance paramyxovirus, Fitzroy River virus, Flaviviridae virus, Flexal mammarenavirus, GB virus C, Gairo virus, Gemycircularvirus, Goose paramyoxiviurs SF02, Great Island virus, Guanarito mammarenavirus, Hantaan virus, Hantavirus Z10, Heartland virus, Hendra virus, Hepatitis A/B/C/E, Hepatitis delta virus, Human bocavirus, Human coronavirus, Human endogenous retrovirus K, Human enteric coronavirus, Human gential-associated circular DNA virus-1, Human herpesvirus 1-8, Human immunodeficiency virus 1/2, Huan mastadenovirus A-G, Human papillomavirus, Human parainfluenza virus 1-4, Human paraechovirus, Human picobirnavirus, Human smacovirus, Ikoma lyssavirus, Ilheus virus, Influenza A-C, Ippy mammarenavirus, Irkut virus, J-virus, JC polyomavirus, Japanses encephalitis virus, Junin mammarenavirus, KI polyomavirus, Kadipiro virus, Kamiti River virus, Kedougou virus, Khujand virus, Kokobera virus, Kyasanur forest disease virus, Lagos bat virus, Langat virus, Lassa mammarenavirus, Latino mammarenavirus, Leopards Hill virus, Liao ning virus, Ljungan virus, Lloviu virus, Louping ill virus, Lujo mammarenavirus, Luna mammarenavirus, Lunk virus, Lymphocytic choriomeningitis mammarenavirus, Lyssavirus Ozernoe, MSSI2\0.225 virus, Machupo mammarenavirus, Mamastrovirus 1, Manzanilla virus, Mapuera virus, Marburg virus, Mayaro virus, Measles virus, Menangle virus, Mercadeo virus, Merkel cell polyomavirus, Middle East respiratory syndrome coronavirus, Mobala mammarenavirus, Modoc virus, Moijang virus, Mokolo virus, Monkeypox virus, Montana myotis leukoenchalitis virus, Mopeia lassa virus reassortant 29, Mopeia mammarenavirus, Morogoro virus, Mossman virus, Mumps virus, Murine pneumonia virus, Murray Valley encephalitis virus, Nariva virus, Newcastle disease virus, Nipah virus, Norwalk virus, Norway rat hepacivirus, Ntaya virus, O′nyong-nyong virus, Oliveros mammarenavirus, Omsk hemorrhagic fever virus, Oropouche virus, Parainfluenza virus 5, Parana mammarenavirus, Parramatta River virus, Peste-des-petits-ruminants virus, Pichande mammarenavirus, Picornaviridae virus, Pirital mammarenavirus, Piscihepevirus A, Procine parainfluenza virus 1, porcine rubulavirus, Powassan virus, Primate T-lymphotropic virus 1-2, Primate erythroparvovirus 1, Punta Toro virus, Puumala virus, Quang Binh virus, Rabies virus, Razdan virus, Reptile bornavirus 1, Rhinovirus A-B, Rift Valley fever virus, Rinderpest virus, Rio Bravo virus, Rodent Torque Teno virus, Rodent hepacivirus, Ross River virus, Rotavirus A-I, Royal Farm virus, Rubella virus, Sabia mammarenavirus, Salem virus, Sandfly fever Naples virus, Sandfly fever Sicilian virus, Sapporo virus, Sathuperi virus, Seal anellovirus, Semliki Forest virus, Sendai virus, Seoul virus, Sepik virus, Severe acute respiratory syndrome-related coronavirus, Severe fever with thrombocytopenia syndrome virus, Shamonda virus, Shimoni bat virus, Shuni virus, Simbu virus, Simian torque teno virus, Simian virus 40-41, Sin Nombre virus, Sindbis virus, Small anellovirus, Sosuga virus, Spanish goat encephalitis virus, Spondweni virus, St. Louis encephalitis virus, Sunshine virus, TTV-like mini virus, Tacaribe mammarenavirus, Taila virus, Tamana bat virus, Tamiami mammarenavirus, Tembusu virus, Thogoto virus, Thottapalayam virus, Tick-borne encephalitis virus, Tioman virus, Togaviridae virus, Torque teno canis virus, Torque teno douroucouli virus, Torque teno felis virus, Torque teno midi virus, Torque teno sus virus, Torque teno tamarin virus, Torque teno virus, Torque teno zalophus virus, Tuhoko virus, Tula virus, Tupaia paramyxovirus, Usutu virus, Uukuniemi virus, Vaccinia virus, Variola virus, Venezuelan equine encephalitis virus, Vesicular stomatitis Indiana virus, WU Polyomavirus, Wesselsbron virus, West Caucasian bat virus, West Nile virus, Western equine encephalitis virus, Whitewater Arroyo mammarenavirus, Yellow fever virus, Yokose virus, Yug Bogdanovac virus, Zaire ebolavirus, Zika virus, or Zygosaccharomyces bailii virus Z viral sequence. Examples of RNA viruses that may be detected include one or more of (or any combination of) Coronaviridae virus, a Picornaviridae virus, a Caliciviridae virus, a Flaviviridae virus, a Togaviridae virus, a Bornaviridae, a Filoviridae, a Paramyxoviridae, a Pneumoviridae, a Rhabdoviridae, an Arenaviridae, a Bunyaviridae, an Orthomyxoviridae, or a Deltavirus. In certain example embodiments, the virus is Coronavirus, SARS, Poliovirus, Rhinovirus, Hepatitis A, Norwalk virus, Yellow fever virus, West Nile virus, Hepatitis C virus, Dengue fever virus, Zika virus, Rubella virus, Ross River virus, Sindbis virus, Chikungunya virus, Borna disease virus, Ebola virus, Marburg virus, Measles virus, Mumps virus, Nipah virus, Hendra virus, Newcastle disease virus, Human respiratory syncytial virus, Rabies virus, Lassa virus, Hantavirus, Crimean-Congo hemorrhagic fever virus, Influenza, or Hepatitis D virus.

In certain example embodiments, the virus may be a plant virus selected from the group comprising Tobacco mosaic virus (TMV), Tomato spotted wilt virus (TSWV), Cucumber mosaic virus (CMV), Potato virus Y (PVY), the RT virus Cauliflower mosaic virus (CaMV), Plum pox virus (PPV), Brome mosaic virus (BMV), Potato virus X (PVX), Citrus tristeza virus (CTV), Barley yellow dwarf virus (BYDV), Potato leafroll virus (PLRV), Tomato bushy stunt virus (TBSV), rice tungro spherical virus (RTSV), rice yellow mottle virus (RYMV), rice hoja blanca virus (RHBV), maize rayado fino virus (MRFV), maize dwarf mosaic virus (MDMV), sugarcane mosaic virus (SCMV), Sweet potato feathery mottle virus (SPFMV), sweet potato sunken vein closterovirus (SPSVV), Grapevine fanleaf virus (GFLV), Grapevine virus A (GVA), Grapevine virus B (GVB), Grapevine fleck virus (GFkV), Grapevine leafroll-associated virus-1, -2, and -3, (GLRaV-1, -2, and -3), Arabis mosaic virus (ArMV), or Rupestris stem pitting-associated virus (RSPaV). In a preferred embodiment, the target RNA molecule is part of said pathogen or transcribed from a DNA molecule of said pathogen.

In certain example embodiments, the virus may be a retrovirus. Example retroviruses that may be detected using the embodiments disclosed herein include one or more of or any combination of viruses of the Genus Alpharetrovirus, Betaretrovirus, Gammaretrovirus, Deltaretrovirus, Epsilonretrovirus, Lentivirus, Spumavirus, or the Family Metaviridae, Pseudoviridae, and Retroviridae (including HIV), Hepadnaviridae (including Hepatitis B virus), and Caulimoviridae (including Cauliflower mosaic virus).

In certain example embodiments, the virus is a DNA virus. Example DNA viruses that may be detected using the embodiments disclosed herein include one or more of (or any combination of) viruses from the Family Myoviridae, Podoviridae, Siphoviridae, Alloherpesviridae, Herpesviridae (including human herpes virus, and Varicella Zozter virus), Malocoherpesviridae, Lipothrixviridae, Rudiviridae, Adenoviridae, Ampullaviridae, Ascoviridae, Asfarviridae (including African swine fever virus), Baculoviridae, Cicaudaviridae, Clavaviridae, Corticoviridae, Fuselloviridae, Globuloviridae, Guttaviridae, Hytrosaviridae, Iridoviridae, Maseilleviridae, Mimiviridae, Nudiviridae, Nimaviridae, Pandoraviridae, Papillomaviridae, Phycodnaviridae, Plasmaviridae, Polydnaviruses, Polyomaviridae (including Simian virus 40, JC virus, BK virus), Poxviridae (including Cowpox and smallpox), Sphaerolipoviridae, Tectiviridae, Turriviridae, Dinodnavirus, Salterprovirus, Rhizidovirus, among oIn an embodiment, a method of diagnosing a species-specific bacterial infection in a subject suspected of having a bacterial infection is described as obtaining a sample comprising bacterial ribosomal ribonucleic acid from the subject; contacting the sample with one or more of the probes described, and detecting hybridization between the bacterial ribosomal ribonucleic acid sequence present in the sample and the probe, wherein the detection of hybridization indicates that the subject is infected with Escherichia coli, Klebsiella pneumoniae, Pseudomonas aeruginosa, Staphylococcus aureus, Acinetobacter baumannii, Candida albicans, Enterobacter cloacae, Enterococcus faecalis, Enterococcus faecium, Proteus mirabilis, Staphylococcus agalactiae, or Staphylococcus maltophilia or a combination thereof.

In certain example embodiments, the infectious agent is a virus. In certain example embodiments, the virus is a DNA virus or an RNA virus. In certain example embodiments, the virus is a double stranded DNA virus, single stranded DNA virus, double-stranded RNA virus, a positive sense RNA virus, a negative sense RNA virus, or a retrovirus (which is inclusive of lentiviruses). In an embodiment the virus is a Group I, Group II, Group III, Group IV, Group V, Group VI, or Group VII virus according to the Baltimore classification system.

In an embodiment, the virus is an RNA virus.

In an embodiment, the RNA virus can infect human and/or non-human vertebrates and is in the family of Birnaviridae, Arteriviridae, Bornaviridae, Nodaviridae, Picobirnaviridae, Reoviridae, Coronaviridae, Astroviridaee, Caliciviridae, Flaviviridae, Hepeviridae, Matonaviridae, Picornaviridae, Togaviridae, Filoviridae, Paramyxoviridae, Pneumoviridae, Rhabdoviridae, Arenaviridae, Hantaviridae, Nairoviridae, Peribunyaviridae, Phenuiviridae, or Orthomyxoviridae.

In an embodiment, the RNA virus can infect a human and/or non-human vertebrates and is in the genus Aquabirnavirus, Avibirnavirus, Blosnavirus, Picobirnavirus, Aquareovirus, Coltivirus, Orthoreovirus, Orbivirus, Rotavirus, Seadornavirus, Orthohepevirus, Piscihepevirus, Alphaartervirus, Lambdaartervirus, Deltavirus, Etaaterivirus, Epsilonaterivirus, Iotaarterivirus, Thetaartereivirus, Zetaartervirius, Betaarterivirus, Gammaatervirus, Kappaarterivirus, Alphacoronavirus, Betacoronavirus, Gammacoronavirus, Deltacoronavirus, Torovirus, Bafinivirus, Ailurivirus, Ampivirus, Aphtovirus, Aquamavirus, Avihepatovirus, Avisivirus, Cardiovirus, Cosavirus, Crohivirus, Dicipivirus, Enterovirus, Erbovirus, Gallivirus, Harkavirus, Hepatovirus, Hunnivirus, Kobuvirus, Kunsagivirus, Limnipivirus, Megrivirus, Mosavirus, Oscivirus, Parechovirus, Pasivirus, Passerivirus, Potamipvirus, Rabovirus, Rosavirus, Sakobuvirus, Salivirus, Sapelovirus, Senecavirus, Sicinivirus, Teschovirus, Torchivirus, Tremovirus, Avastrovirus, Mamastrovirus, Lagovirus, Nebovirus, Norovirus, Sapovirus, Vesivirus, Flavivirus, Hepacivirus, Pegivirus, Pestivirus, Rubivirus, Alphanodavirus, Betanodivirus, Alphavirus, Orthobornavirus, Carbovirus, Nyavirus, Ephemerovirus, Ephemerovirus, Hapavirus, Ledantevirus, Perhabdovirus, Sprivivirus, Tibrovirus, Tupavirus, Vesiculovirus, Cuevavirus, Ebolavirus, Marburgvirus, Aquaparamyxovirus, Avulavirus, Ferlavirus, Henipavirus, Morbillivirus, Respirovirus, Rubulavirus, Metapneumonvirus, Orthopneumonvirus, Hartmanivirus, Mammarenvirus, Reptarenavirus, Orthohantavirus, Orthonairovirus, Phlebovirus, Alphainfluenzavirus, Betainfluenzavirus, Gammainfluenzavirus, Deltainfluenzavirus, Thogotovirus, Isavirus, Quaranjavirus, Orthobunyavirus, Sunshinevirus, Tilapinevirus, or Deltavirus.

In an embodiment, the RNA virus can infect a plant and is in the family Amalgaviridae, Endornaviridae, Partitiviridae, Reoviridae, Secoviridae, Alpha-flexiviridae, Beta-flexiviridae, Tymoviridae, Virgaviridae, Bromoviridae, Closteroviridae, Luteoviridae, Potyviridae, Solemoviridae, Tombusviridae, Benyviridae, Rhabdoviridae, Fimoviridae, Phenuiviridae, Tospoviridae, Aspiviridae, Avsunviroidae, or Pospiviroidae.

In an embodiment, the RNA virus can infect a plant and is in the genus Amalgavirus, Alphaendoma, Alphapartitivirus, Betapartitivrus, Deltapartitivirus, Fijivirus, Oryzavirus, Phytoreovirus, Cheravirus, Comovirus, FAbavirus, Nepovirus, Sadwavirus, Sequivirus, Torradovirus, Waikavirus, Allexivirus, Mandarivirus, Platpuvirus, Potexivirus, Lolavirus, Capillovirus, Carlavirus, Chordovirus, Citrivirus, Divavirus, Foveavirus, Prunevirus, Robigovirus, Tepovirus, Trichovirus, Vitivirus, Maculavirus, Marafivirus, Tymovirus, Furovirus, Goravirurs, Hordeivirus, Pecluvirus, Pmovirus, Tobamovirus, Tobravirus, Alfamovirus. Anulavirus, Bromovirus, Cucumovirus, Ilarviurs, Oleavirus, Ampelovirus, Closterovirus, Crinivirus, Velarivirus, Enamovirus, Leutovirus, Polerovirus, Bevemovirus. Brambyvirus, Bymovirus, Ipomovirus, Macluravirus, Poacevirus, Potyvirus, Roymovirus, Rymovirus, Tritimovirus, Polemovirus, Sobmovirus, Alphacamovirus, Aplhanecrovirus, Aureusvirus, Avenavirus, Betavarmovirus, Betanecrovirus, Dianthovirus, Gallantivirus, Gamma-carmovirus, Macanavirus, Machlomovirus, Panicovirus, Pelarspovirus, Umbravirus, Zeavirus, Benyvirus, Albetovirus, Aumavirus, Blunervirus, Cilevirus, Higrevirus, Idaeovirus, Ourmiavirus, Papanivirus, Sinavirus, Virtovirus, Cytorhabdovirus, Dichorhavirus, Nucleorhabdo-virus, Varicosavirus, Emaravirus, Tenuivirus, Orthotospovirus, Ophiovirus, Avsunvirioid, Elaviroid, Pelamoviroid, Apscaviroid, Cocadviroid, Coleviroid, Hostuviroid, or Pospiviroid.

In an embodiment, the virus is a DNA virus.

In an embodiment, the DNA virus can infect humans and/or non-human vertebrates and is in the family Herpesviridae, Alloherpesviridae, Adenoviridae, Papillomaviridae, Polomaviridae, Asfarviridae, Iridoviridae, Poxviridae, Anelloviridae, Circoviridae, Genomoviridae, or Parvoviridae.

In an embodiment, the DNA virus can infect humans and/or non-human vertebrates and is in the genus Simplexvirus, Varicellovirus, Mardivirus, Scutavirus, Iltovirus, Cytomegalovirus, Muromegalovirus, Roseolivirus, Proboscivirus, Lymphocrypto-virus, Rhadinovirus, Macavirus, Percavirus, Batrachovirus, Cyprinivirus, Ictalurivirus, Salmonivirus, Mastadenovirus, Aviadenovirus, Atadenovirus, Ichtadenovirus, Siadenovirus, Alphapapillomavirus, Betapapillomavirus, Chipapillomavirus, Deltapapillomavirus, Dyochipapillomavirus, Dyoepsilonpapillomavirus, Dyodeltapapillomavirus, Dyoetapapillomavirus, Dyiotapapillomavirus, Dyokappapapillomavirus, Dyonupapillomavirus, Dyophipapillomavirus, Dyorhopapillomavirus, Dyothetapapillomavirus, Dyolambdapapillomavirus, Dyomupapillomavirus, Dyoomegapapillomavirus, Dyopipapillomavirus, Dyoomikronpapillomavirus, Dyopsipapillomavirus, Dyosigmapapillomavirus, Dyotaupapillomavirus, Dyoupsilonpapillomavirus, Dyoxipapillomavirus, Dyozetapapillomavirus, Epsilonpapillomavirus, Etapapillomavirus, Gammapapillomavirus, Iotapapillomavirus, Kappapapillomavirus, Lambdapapillomavirus, Mupapillomavirus, Nupapillomavirus, Omegapapillomavirus, Omikronpapillomavirus, Phipapillomavirus, Psipapillomavirus, Rhopapillomavirus, Sigmapapillomavirus, Taupapillomavirus, Thetapapillomavirus, Treisdeltapapillomavirus, Treisiotapapillomavirus, Treisepsilonpapilomavirus, Treiskappapapillomavirus, Treisthetapapillomavirus, Treiszetapapillomavirus, Treiszetapapillomavirus, Upsilonpapillomavirus, Xipapillomavirus, Zetapapillomavirus, Alefpapillomavirus, Alpha-polyomavirus, Beta-polyomavirus, Gamma-polyomavirus, Delta-polyomavirus, Asfivirus, Lymphocystivirus, Megalocytivirus, Ranavirus, Avipoxvirus, Capripoxvirus, Cervidopoxvirus, Crocodylidpoxvirus, Leporipoxvirus, Molluscopoxvirus, Orthopoxvirus, Parpoxvirus, Suipoxvirus, Yatapoxvirus, Alphatorquevirus, Betatorquevirus, Gammatorquevirus, Deltatorquevirus, Epsilontorquevirus, Lambdatorquevirus, Kappatorquevirus, Zetatorquevirus, Etatorquevirus, Thetatorquevirus, Iotatorquevirus, Gyrovirus, Circovirus, Cyclovirus, Gemycicular-virus, Gemygorvirus, Gemykibivirus, Gemykolovirus, Gemykrogvirus, Gemykroznavirus, Gemytondvirus, Gemyvongvirus, Amdoparvovirus, Aveparvovirus, Protoparvovirus, Copiparvoirus, Erythroparvovirus, Dependoparvovirus, Tetraparvovirus, or Bocaparvovirus.

In an embodiment, the virus is a retrovirus. Exemplary retroviruses include, but are not limited to, any of those of the genus Alpharetrovirus, Betaretrovirus, Gammaretrovirus, Deltaretrovirus, Epsilonretrovirus, Lentivirus, Spumavirus, or the Family Metaviridae, Pseudoviridae, and Retroviridae (including HIV), Hepadnaviridae (including Hepatitis B virus), and Caulimoviridae (including Cauliflower mosaic virus).

In certain example embodiments, the virus is a coronavirus, an Ebola virus, measles, SARS, Chikungunya virus, Marburg, MERS, Dengue, Lassa, influenza, rhabdovirus, HIV, a hepatitis virus (including hepatitis A, B, C, D, or E), an influenza virus (including an influenza A or influenza B), a human respiratory syncytial virus, Sudan ebola virus, Bundibugyo virus, Tai Forest ebola virus, Reston ebola virus, Achimota virus, Aedes flavivirus, Aguacate virus, Akabane virus, Alethinophid reptarenavirus, Allpahuayo mammarenavirus, Amapari mmarenavirus, Andes virus, Apoi virus, Aravan virus, Aroa virus, Arumwot virus, Atlantic salmon paramyxovirus, Australian bat lyssavirus, Avian bornavirus, Avian metapneumovirus, Avian paramyxoviruses, penguin or Falkland Islandsvirus, BK polyomavirus, Bagaza virus, Banna virus, Bat herpesvirus, Bat sapovirus, Bear Canon mammarenavirus, Beilong virus, Betacoronavirus, Betapapillomavirus 1-6, Bhanja virus, Bokeloh bat lyssavirus, Borna disease virus, Bourbon virus, Bovine hepacivirus, Bovine parainfluenza virus 3, Bovine respiratory syncytial virus, Brazoran virus, Bunyamwera virus, Caliciviridae virus. California encephalitis virus, Candiru virus, Canine distemper virus, Canine pneumovirus, Cedar virus, Cell fusing agent virus, Cetacean morbillivirus, Chandipura virus, Chaoyang virus, Chapare mammarenavirus, Chikungunya virus, Colobus monkey papillomavirus, Colorado tick fever virus, Cowpox virus, Crimean-Congo hemorrhagic fever virus, Culex flavivirus, Cupixi mammarenavirus, Dengue virus, Dobrava-Belgrade virus, Donggang virus, Dugbe virus, Duvenhage virus, Eastern equine encephalitis virus, Entebbe bat virus, Enterovirus A-D, European bat lyssavirus 1-2, Eyach virus, Feline morbillivirus, Fer-de-Lance paramyxovirus, Fitzroy River virus, Flaviviridae virus, Flexal mammarenavirus, GB virus C, Gairo virus, Gemycircularvirus, Goose paramyxovirus SF02, Great Island virus, Guanarito mammarenavirus, Hantaan virus, Hantavirus Z10, Heartland virus, Hendra virus, Hepatitis A/B/C/E, Hepatitis delta virus, Human bocavirus, Human coronavirus, Human endogenous retrovirus K, Human enteric coronavirus, Human genital-associated circular DNA virus-1, Human herpesvirus 1-8, Human mastadenovirus A-G, Human papillomavirus, Human parainfluenza virus 1-4, Human paraechovirus, Human picornavirus, Human smacovirus, Ikoma lyssavirus, Ilheus virus, Influenza A-C, Ippy mammarenavirus, Irkut virus, J-virus, JC polyomavirus, Japanese encephalitis virus, Junin mammarenavirus, KI polyomavirus, Kadipiro virus, Kamiti River virus, Kedougou virus, Khujand virus, Kokobera virus, Kyasanur forest disease virus, Lagos bat virus, Langat virus, Lassa mammarenavirus, Latino mammarenavirus, Leopards Hill virus, Liao ning virus, Ljungan virus, Lloviu virus, Louping ill virus, Lujo mammarenavirus, Luna mammarenavirus, Lunk virus, Lymphocytic choriomeningitis mammarenavirus, Lyssavirus Ozernoe, MSSI2Y225 virus, Machupo mammarenavirus, Mamastrovirus 1, Manzanilla virus, Mapuera virus, Marburg virus, Mayaro virus, Measles virus, Menangle virus, Mercadeo virus, Merkel cell polyomavirus, Middle East respiratory syndrome coronavirus, Mobala mammarenavirus, Modoc virus, Moijang virus, Mokolo virus, Monkeypox virus, Montana myotis leukoenchalitis virus, Mopeia lassa virus reassortant 29, Mopeia mammarenavirus, Morogoro virus, Mossman virus, Mumps virus, Murine pneumonia virus, Murray Valley encephalitis virus, Nariva virus, Newcastle disease virus, Nipah virus, Norwalk virus, Norway rat hepacivirus, Ntaya virus, O′nyong-nyong virus, Oliveros mammarenavirus, Omsk hemorrhagic fever virus, Oropouche virus, Parainfluenza virus 5, Parana mammarenavirus, Parramatta River virus, Peste-des-petits-ruminants virus, Pichande mammarenavirus, Picornaviridae virus, Pirital mammarenavirus, Piscihepevirus A, Porcine parainfluenza virus 1, porcine rubulavirus, Powassan virus, Primate T-lymphotropic virus 1-2, Primate erythroparvovirus 1, Punta Toro virus, Puumala virus, Quang Binh virus, Rabies virus, Razdan virus, Reptile bornavirus 1, Rhinovirus A-B, Rift Valley fever virus, Rinderpest virus, Rio Bravo virus, Rodent Torque Teno virus, Rodent hepacivirus, Ross River virus, Rotavirus A-I, Royal Farm virus, Rubella virus, Sabia mammarenavirus, Salem virus, Sandfly fever Naples virus, Sandfly fever Sicilian virus, Sapporo virus, Sathuperi virus, Seal anellovirus, Semliki Forest virus, Sendai virus, Seoul virus, Sepik virus, Severe acute respiratory syndrome-related coronavirus, Severe fever with thrombocytopenia syndrome virus, Shamonda virus, Shimoni bat virus, Shuni virus, Simbu virus, Simian torque teno virus, Simian virus 40-41, Sin Nombre virus, Sindbis virus, Small anellovirus, Sosuga virus, Spanish goat encephalitis virus, Spondweni virus, St. Louis encephalitis virus, Sunshine virus, TTV-like mini virus, Tacaribe mammarenavirus, Taila virus, Tamana bat virus, Tamiami mammarenavirus, Tembusu virus, Thogoto virus, Thottapalayam virus, Tick-borne encephalitis virus, Tioman virus, Togaviridae virus, Torque teno canis virus, Torque teno douroucouli virus, Torque teno felis virus, Torque teno midi virus, Torque teno sus virus, Torque teno tamarin virus, Torque teno virus, Torque teno zalophus virus, Tuhoko virus, Tula virus, Tupaia paramyxovirus, Usutu virus, Uukuniemi virus, Vaccinia virus, Variola virus, Venezuelan Vesicular stomatitis Indiana virus, WU Polyomavirus, Wesselsbron virus, West Caucasian bat virus, West Nile virus, Western equine encephalitis virus, Whitewater Arroyo mammarenavirus, Yellow fever virus, Yokose virus, Yug Bogdanovac virus, Zaire ebolavirus, Zika virus, or Zygosaccharomyces bailii virus Z viral sequence, or a combination thereof.

In certain example embodiments, the virus is a coronavirus. In certain example embodiments, the virus is SARS-COV-2. In an embodiment, the SARS-COV-2 is strain G, strain GR, strain GH, stain L, strain V, or strain S, or a variant thereof, or a mutant thereof (see e.g. Daniele Mercatelli, Federico M. Giorgi. Geographic and Genomic Distribution of SARS-CoV-2 Mutations. Frontiers in Microbiology, 2020; 11 DOI: 10.3389/fmicb.2020.01800, particularly at e.g. Tables 1 and 2, Supplementary Files 6-7 and 9).

### Mitochondrial Diseases

Some of the most challenging mitochondrial disorders arise from mutations in mitochondrial DNA (mtDNA), a high copy number genome that is maternally inherited. In an embodiment, mtDNA mutations can be modified using a composition of the present invention described herein. In an embodiment, the mitochondrial disease that can be diagnosed, prognosed, treated, and/or prevented can be MELAS (mitochondrial myopathy encephalopathy, and lactic acidosis and stroke-like episodes), CPEO/PEO (chronic progressive external ophthalmoplegia syndrome/progressive external ophthalmoplegia), KSS (Kearns-Sayre syndrome), MIDD (maternally inherited diabetes and deafness), MERRF (myoclonic epilepsy associated with ragged red fibers), NIDDM (noninsulin-dependent diabetes mellitus), LHON (Leber hereditary optic neuropathy), LS (Leigh Syndrome) an aminoglycoside induced hearing disorder, NARP (neuropathy, ataxia, and pigmentary retinopathy), Extrapyramidal disorder with akinesia-rigidity, psychosis and SNHL, Nonsyndromic hearing loss a cardiomyopathy, an encephalomyopathy, Pearson's syndrome, a disease identified as being caused or attributed to a mtDNA mutation set forth at mitomap.org, or a combination thereof.

In an embodiment, the mtDNA of a subject can be modified in vivo or ex vivo. In an embodiment, where the mtDNA is modified ex vivo, after modification the cells containing the modified mitochondria can be administered back to the subject. In an embodiment, the engineered therapeutic polynucleotide is of correcting an mtDNA mutation such as any one or more of those that can be found at mitomap.org.

In an embodiment, at least one of the one or more mtDNA mutations is selected from the group consisting of: A3243G, C3256T, T3271C, G1019A, A1304T, A15533G, C1494T, C4467A, T1658C, G12315A, A3421G, A8344G, T8356C, G8363A, A13042T, T3200C, G3242A, A3252G, T3264C, G3316A, T3394C, T14577C, A4833G, G3460A, G9804A, G11778A, G14459A, A14484G, G15257A, T8993C, T8993G, G10197A, G13513A, T1095C, C1494T, A1555G, G1541A, C1634T, A3260G, A4269G, T7587C, A8296G, A8348G, G8363A, T9957C, T9997C, G12192A, C12297T, A14484G, G15059A, duplication of CCCCCTCCCC-tandem (SEQ ID NO: 25) repeats at positions 305-314 and/or 956-965, deletion at positions from 8,469-13,447, 4,308-14,874, and/or 4,398-14,822, 961ins/delC, the mitochondrial common deletion (e.g. mtDNA 4,977 bp deletion), and combinations thereof.

In an embodiment, the mitochondrial mutation can be any mutation as set forth in or as identified by use of one or more bioinformatic tools available at Mitomap available at mitomap.org. Such tools include, but are not limited to, “Variant Search, aka Market Finder”, Find Sequences for Any Haplogroup, aka “Sequence Finder”, “Variant Info”, “POLG Pathogenicity Prediction Server”, “MITOMASTER”, “Allele Search”, “Sequence and Variant Downloads”, “Data Downloads”. MitoMap contains reports of mutations in mtDNA that can be associated with disease and maintains a database of reported mitochondrial DNA Base Substitution Diseases: rRNA/tRNA mutations.

In an embodiment, the method includes delivering a CRISPR-Cas system and/or a component thereof to a cell, and more specifically one or more mitochondria in a cell, allowing the CRISPR-Cas system and/or component thereof to modify one or more target polynucleotides in the cell, and more specifically one or more mitochondria in the cell. The target polynucleotides can correspond to a mutation in the mtDNA, such as any one or more of those described herein. In an embodiment, the modification can alter a function of the mitochondria such that the mitochondria functions normally or at least is/are less dysfunctional as compared to an unmodified mitochondria. Modification can occur in vivo or ex vivo. Where modification is performed ex vivo, cells containing modified mitochondria can be administered to a subject in need thereof in an autologous or allogenic manner.

Further embodiments are illustrated in the following Examples which are given for illustrative purposes only and are not intended to limit the scope of the invention.

### EXAMPLES

Now having described the embodiments of the present disclosure, in general, the following Examples describe some additional embodiments of the present disclosure. While embodiments of the present disclosure are described in connection with the following examples and the corresponding text and figures, there is no intent to limit embodiments of the present disclosure to this description. On the contrary, the intent is to cover all alternatives, modifications, and equivalents included within the spirit and scope of embodiments of the present disclosure. The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

### Example 1—Genome-Wide Models of Cis-Regulatory Activity and Synthetic Enhancers

The gene is a fundamental unit of information essential for life, and a genome is the collection of genes and regulatory instructions that compose the “blueprint” for the life of an organism. It is replicated and inherited both across generations of a species and during cellular di-vision and differentiation within multi-cellular organisms. The canonical coding gene executes its role when its DNA sequence is transcribed to RNA, and RNA is translated to a protein that exerts a biochemical or physical function. In metazoans, multi-cellular animals composed of differentiated cell types, tight regulation of the genome allows specialized cells to produce the necessary proteins for executing their function. Proper protein production through exquisitely controlled gene regulation of a given cell is essential to an organism's healthy development and continued survival.

Chromatin organization and gene regulation in eukaryotes is a complex process partly governed by the interactions of trans-acting factors, such as transcription factors (TFs), with cis-regulatory elements (CREs), which are DNA modules in the genome that specify the rules for gene regulation. Four important classes of CREs are promoters, enhancers, silencers, and insulators (Hardison, R. C. & Taylor, J. (2012). Genomic approaches towards finding cis-regulatory modules in animals. Nature Reviews Genetics, 13 (7), 469-483). Promoters are a core component of protein coding genes, generally located directly upstream of every transcription start site, where transcription is initiated through the binding of transcription factors (TFs) and the assembly of the RNA polymerase (Haberle, V. & Stark, A. (2018). Eukaryotic core promoters and the functional basis of transcription initiation. Nature Reviews Molecular Cell Biology, 19 (10), 621-637). Enhancers are short sequences composed of one or more TF binding sites that recruit co-activators of gene expression and, similar to promoters, participate in transcription initiation (Hardison, R. C. & Taylor, J. (2012). Genomic approaches towards finding cis-regulatory modules in animals. Nature Reviews Genetics, 13 (7), 469-483; Kim, T.-K. & Shiekhattar, R. (2015). Architectural and functional commonalities between enhancers and promoters. Cell, 162 (5), 948-959; and Long, H. K., Prescott, S. L., & Wysocka, J. (2016). Ever-changing landscapes: Transcriptional enhancers in development and evolution. Cell, 167 (5), 1170-1187). The two features that distinguish promoters from enhancers are: (i) enhancers can act over highly variable distances (kilobase to megabase scale), and (ii) one enhancer can interact with multiple genes and vice versa (Fulco, C. P., et al. (2019). Activity-by-contact model of enhancer-promoter regulation from thousands of crispr perturbations. Nature Genetics, 51 (12), 1664-1669). Few silencers have been comprehensively validated in vivo, so their prevalence is debated, but they are thought to be similar to enhancers except that they recruit repressors of transcription (Hardison, R. C. & Taylor, J. (2012). Genomic approaches towards finding cis-regulatory modules in animals. Nature Reviews Genetics, 13 (7), 469-483). Insulators establish boundaries for the action of other long-range CREs (Hardison, R. C. & Taylor, J. (2012). Genomic approaches towards finding cis-regulatory modules in animals. Nature Reviews Genetics, 13 (7), 469-483). Together, complex CRE and gene interaction networks are foundational to fine spatio-temporal tuning of gene expression, and decades of research show that these mechanisms enabled the development of morphologically complex organisms.

Massively parallel reporter assays (MPRAs) directly characterize cis-regulatory function of DNA sequences with the sensitivity required to measure the impacts of genetic variants accurately. However, it remains intractable to test every element in the human genome using MPRAs. Applicant presents Malinois, a convolutional neural network model of MPRA activity using data from 3 cell lines: erythroleukemia (K562), hepactocellular carcinoma (HepG2), and neuroblastoma (SK-N-SH) cells. Malinois generalizes well to held-out sequences (Pearson's r=0.88) and can simulate data from various assay designs, including MPRA tiling, saturation mutagenesis, and variant effect screens. Malinois infers a genome-wide map of regulatory function, which is well associated with DNase and H3K27ac signals. Applicant also shows that Malinois variant effect predictions (VEPs) are more concordant with MPRA allelic skew measurements than VEPs provided by a highly accurate chromatin state model. Applicant analyzed 15,634,266 non-coding somatic mutations identified in human cancers and found variants near genes implicated in cancer disproportionately affect predicted regulatory elements. Applicant also generated VEPs for 707,933,985 human germline variants in gnomAD, observing variants at conserved nucleotides in regulatory elements exhibit significantly higher functional impact. Finally, Applicant harnessed Malinois to design tens of thousands of synthetic cell type-specific regulatory elements ab initio. These synthetic sequences, which have no significant match in the genome, exhibit high MPRA-measured cell type specificity, dramatically outperforming DNase I Hypersensitivity (DHS) or Malinois informed selection of enhancer sequences from the genome.

Introduction. Quantifying the gene-regulatory potential of DNA at nucleotide resolution remains a difficult problem in genomics. This limited understanding of “regulatory grammar”—the complex pat-tern of sequences that interact with transcription factors (TFs) to control gene expression-hinders interpretation of human genetic variation. The past decade has seen acceleration of experimental tools to interrogate the genome 64 alongside rapid adoption of cutting-edge machine learning (ML) methods to model chromatin state to overcome this hurdle [6], [163], [77], [52], [87], [113], [76]. Today, there are several models that can infer TF binding, DNA accessibility, transcription initiation, and histone modifications for hundreds of cell types from DNA sequence alone [164].

The stunning accuracy of recent ML models enables the in silico interpretation of genetic variants by way of predicted changes in chromatin state. Expression quantitative trait loci (eQTL) are genetic variants that explain differences in gene expression between tissue samples collected from different individuals [61], [62] and can serve as empirical positive controls for variant effect prediction (VEP). Several studies show ML model-based VEP can accurately distinguish expression quantitative trait loci (eQTL) from negative control variants [6],[163] and correlates significantly with eQTL summary statistics [6], [76]. However, as these models predict changes in chromatin state not regulatory potential of DNA sequence, there is an opportunity to further improve VEP by training models on direct functional characterizations of CREs.

While mapping biochemical markers associated with CRE location and function using techniques such as DNase I Hypersensitivity (DHS) and H3K27ac ChIP-seq, respectively, are useful to identify candidate CREs [64], direct activity characterization is essential to quantify function ([35], [43], [44], [100], [118], [141], [147]). Episomal reporter assays are a crucial tool to validate the potential of a DNA regulatory element to regulate gene expression [93], [159]. Recently, these methods have been supercharged to expand throughput dramatically [100], [78]. Technical improvements to DNA microarray synthesis have enabled the simultaneous programming of 100,000s of 150-250 bp DNA elements. Massively parallel reporter assays (MPRAs) insert these synthesized elements into barcoded reporter constructs which are transfected into cells. High-throughput sequencing of the barcodes is then used to simultaneously measure activity and identify each element in the assay (FIG. 1A). MPRAs are now used for targeted functional characterization of hundreds of thousands of CREs and because of their programmability, can quantify the effects of sequence perturbation on CRE function at nucleotide resolution [100], [141], [147], [82]. MPRAs are now widely used to rapidly expand Applicant's understanding of the non-coding genome using direct measurements of regulatory element function. Given the performance and scale of MPRAS, they provide an exciting resource to build direct models of CREs. Modestly accurate deep learning models have been used to extract biologically meaningful patterns from early MPRA data [102]. However, with the recent release of Phase 4 of ENCODE, Applicant now has the necessary volume of high-quality MPRA data to generate sufficiently accurate models to interpret individual regulatory elements, characterize putative causal alleles, and generate synthetic CREs.

While mapping biochemical markers associated with CRE location and function using techniques such as DNase I Hypersensitivity (DHS) and H3K27ac ChIP-seq, respectively, are useful to identify candidate CREs [64], direct activity characterization is essential to quantify function [35], [43], [44], [100], [118], [141], [147]. Episomal reporter assays are a crucial tool to validate the potential of a DNA regulatory element to regulate gene expression [93], [159]. Recently, these methods have been supercharged to expand throughput dramatically [100], [78]. Technical improvements to DNA microarray synthesis have enabled the simultaneous programming of 100,000s of 150-250 bp DNA elements. Massively parallel reporter assays (MPRAs) insert these synthesized elements into barcoded reporter constructs which are transfected into cells. High-throughput sequencing of the barcodes is then used to simultaneously measure activity and identify each element in the assay (FIG. 1A). MPRAs are now used for targeted functional characterization of hundreds of thousands of CREs and because of their programmability, can quantify the effects of sequence perturbation on CRE function at nucleotide resolution [100], [141], [147], [82]. MPRAs are now widely used to rapidly expand Applicant's understanding of the non-coding genome using direct measurements of regulatory element function. Given the performance and scale of MPRAS, they provide an exciting resource to build direct models of CREs. Modestly accurate deep learning models have been used to extract biologically meaningful patterns from early MPRA data [102]. However, with the recent release of Phase 4 of ENCODE, Applicant now has the necessary volume of high-quality MPRA data to generate sufficiently accurate models to interpret individual regulatory elements, characterize putative causal alleles, and generate synthetic CREs.

Results. Malinois accurately predicts regulatory activity. Applicant set out to design a highly accurate model of DNA regulatory activity measured by MPRAs of short sequences (≤200nt) (FIG. 1A). This can be framed as a multi-task regression problem using inputs with consistent dimensions. Applicant collected data from a cohort of MPRA experiments conducted by a single lab using a consistent library design strategy to avoid technical confounding effects. To enable Applicant's model to learn the impact of sequence variation on CRE activity, Applicant trained on an MPRA containing fine-mapped GWAS alleles from the UK Biobank and GTEx projects [134]. This data set is composed of ˜400, 000 pairs of sequences, the vast majority of which diverge by one base pair. All sequences originating from chromosomes 7, 13, 19, 21, and X were held out from the training set to prevent closely related sequences from contaminating Applicant's performance estimates on the held out test set. In total Applicant's model is trained using roughly 66 Mb of sequence derived from the genome and tested by MPRA.

Applicant implemented a neural-network architecture search to automatically test modifications on the original Basset design [77]. Applicant used Bayesian Optimization to select the best final neural-network architecture and optimize hyperparameters for training a model on MPRA data (Methods) [135]. The resulting model, Malinois, provides accurate predictions of MPRA activity in K562, HepG2, and SK-N-SH cells (FIG. 1B, Pearson's r≥0.87 and Spearman's ρ>0.80). Malinois performs favorably compared to MPRA-DragoNN, the prior state-of-the-art for MPRA prediction in K562 and HepG2 (Spearman's ρ=0.14 0.28) [102]. This large improvement is due in most part to the higher experimental reproducibility in Applicant's data set (Spearman's ρ>0.90) compared to the Sharpr-MPRA data (average Spearman's ρ=0.40) [35,102].

Malinois predicts MPRA genome-wide. MPRAs are targeted, high-resolution, and reproducible assays, but lack enough throughput to provide dense, genome-wide maps of regulatory activity. Thus, Applicant assessed if Malinois could extrapolate MPRA signal genome-wide. First Applicant tested if Malinois could reproduce the results of an MPRA assay in K562 to test every nucleotide from a 2 Mb region on Chromosome X surrounding the GATA1 gene tiled at 50 bp resolution using 200 bp oligos (FIG. 2A). Malinois predictions were highly correlated (Pearson's r=0.91) with the empirically observed signal in this screen, approaching the reproducibility between experimental replicates (Pearson's r=0.99) (FIG. 2B). Predictive accuracy is further improved in regions with high chromatin accessibility where active CREs are more likely present, resulting in improved signal: noise ratios (FIG. 2C-2D). Malinois was trained using a low-resolution library in which two overlapping oligos were used to test each element, however, the high concordance to tiling studies suggests Malinois will still generate accurate high-resolution genome-wide prediction maps.

Next, Applicant explored simulated patterns of MPRA activity genome-wide using 50 bp tiled Malinois predictions. Applicant examined if Malinois predictions for K562 were concordant with DHS and H3K27ac ChIP signals, the canonical biochemical marks for active CREs and enhancers, respectively. Applicant found chromosome-wide correlation between Malinois and DHS can vary substantially (Pearson's r=0.2-0.6), while correlation of Malinois with H3K27ac is low (Pearson's r≤0.18) (FIG. 3A). Low genome-wide correlations can be difficult to interpret because Malinois evaluates a sequence's potential to regulate gene expression disregarding chromatin accessibility. Additionally, most nucleotides in the genome have low Malinois, DHS, and H3K27ac scores, resulting in poor signal: noise. H2K27ac poses particular challenges because: (i) it is a diffuse marker and, (ii) can be depleted directly at CREs where active TF binding causes general histone displacement [78].

Based on Applicant's results at the GATA1 locus, Applicant homed in on peaks to improve the signal to noise ratio. Applicant also restricted this analysis to Chromosome 7 to avoid conflicts with the training data. Applicant found Malinois predictions to be significantly higher within annotated DHS and H3K27ac peaks (FIG. 3B, Welch's t-test, p≤10-300). Self-transcribing active regulatory region sequencing (STARR-seq) is another reporter assay that enables genome-wide functional characterization of enhancer activity, albeit at lower resolution than MPRA. Similar to DHS and H3K27ac, Malinois predictions were significantly higher inside STARR-seq peaks (FIG. 3B, Welch's t-test, p≤10-300). Applicant further scrutinized signal patterns from Malinois, STARR-seq, DHS, and H3K27ac at all DHS peaks on Chromosome 7 to confirm reasonable bp-resolution patterns in Malinois signal. DHS signal is high in these regions, as expected, and overlaps with a dip in H3K27ac signal which is caused by general histone depletion rather than de-enrichment of H3K27ac, specifically (FIG. 3C). This, combined with positive STARR-seq signal in the visualized regions indicate these are likely enhancers. Accordingly, Malinois predictions are generally high at these DHS sites. These results show Malinois predictions are a credible indicator of CRE function genome-wide.

Malinois identifies functional effects of genetic variants. There are more candidate variants responsible for phenotypic diversity in humans than can possibly be interrogated experimentally [1], [42], [67], [75]. Therefore, it is critical to develop precise in silico methods to prioritize genetic variants for functional characterization. Applicant converted MPRA activity predictions into variant effect predictions (VEPs) by computing the differences in predicted activity between sequences containing the alternate allele and sequences containing the reference allele. Here Applicant defines “allelic skew” as the difference in a measurement or prediction between alternate and reference alleles. Applicant compared Malinois VEPs to an MPRA saturation mutagenesis of PKIR, F9, and LDLR promoters and a SORTI enhancer from the CAGI5 competition data set (FIG. 4A-4D).

Overall, Malinois VEPs are well correlated with empirically measured MPRA allelic skews, on average matching previous state-of-the-art results computed by Enformer (Table 7 shows Pearson correlation coefficients of MPRA saturation mutagenesis screens with in silico saturation mutagenesis using Malinois or Enformer) [6]. While encouraging, these results focus on dissecting the activity of well characterized promoters and enhancers where Applicant expected to see an enrichment of variants that have an effect on expression. Effective methods for variant prioritization must make accurate predictions for solitary variants scattered throughout the genome.

TABLE 7

| Gene | Malinois | Enformer |
|---|---|---|
| PKLR | 0.70 | 0.79 |
| F9 | 0.69 | 0.59 |
| LDLR | 0.59 | 0.58 |
| SORT1 | 0.53 | 0.52 |

The MPRA data set that Applicant collected from ENCODE to train and test Malinois is predominately composed of reference/alternate allele pairs from the UK Biobank and GTEx, enabling us to further scrutinize VEP accuracy beyond known promoters and enhancers, and quantify the effectiveness of a model for variant prioritization. Applicant compared VEPs calculated by Malinois for 4000 alleles tested on Chromosome 7 with empirical MPRA allelic skew measurements (FIG. 5A). For comparison, Applicant also calculated VEPs for all of these variants using Enformer [6] (FIG. 5B). Applicant found Malinois to be substantially more accurate than Enformer for predicting variant effects measured by MPRA (FIG. 5C). Malinois directly models MPRA and is better suited to predict the outcome of a functional characterization experiment than Enformer which was trained on bio-chemical features indirectly associated with CRE function.

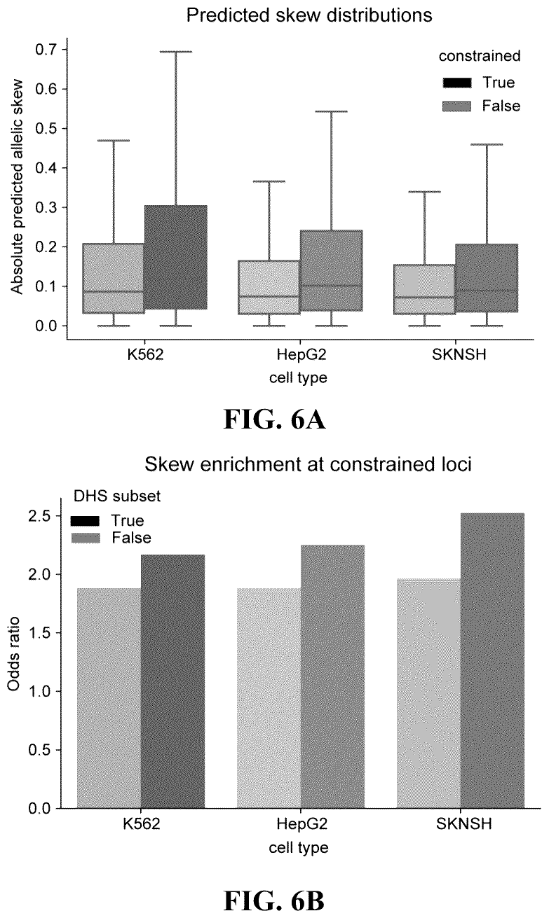

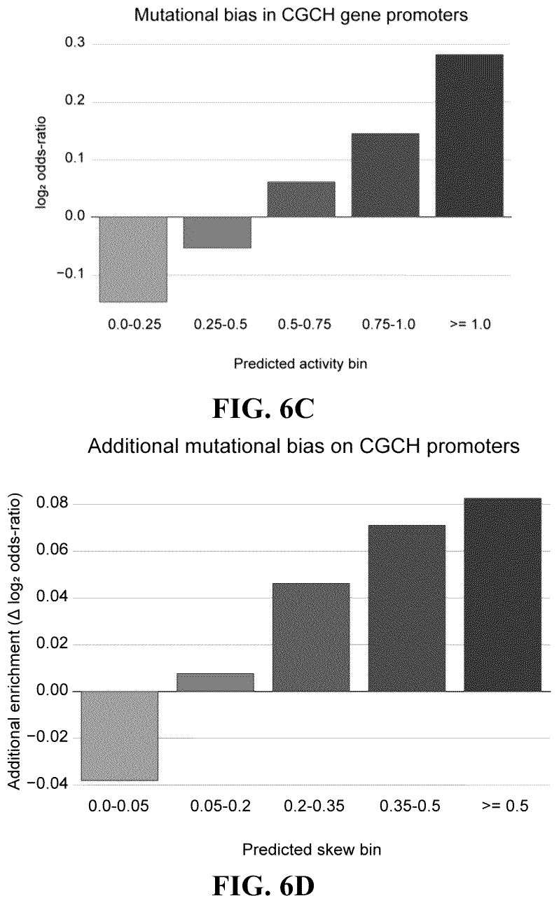

Applicant used Malinois to create a reference set of MPRA allelic skew predictions in K562, HepG2, and SK-N-SH for 707,933,985 variants from the Genome Aggregation Database (gnomAD) [75]. The Zoonomia Consortium recently provided nucleotide resolution estimates of evolutionary constraint based on a comparative analysis of 241 mammals; these phyloP scores can pinpoint important nucleotides for CRE function [49]. In each cell type, Applicant showed variants in open chromatin have larger impacts on allelic skew when they perturb conserved versus non-conserved nucleotides (FIG. 6A, Welch's t-test, p≤10-300 for all 3 cell types). This increased allelic skew at conserved positions translates to an enrichment of strong allelic skew variants (i.e., |skew|≥1, FIG. 6B, Fisher's ex-act test p≤10-80 for all conditions). Overall, Applicant found Malinois remains concordant with biological indicators of function, further encouraging us to use Malinois for variant prioritization.

Non-coding driver mutations are relatively rare in cancer and are difficult to identify due to the high background of passenger mutations [18], [120]. Functional characterization models can thus help us prioritize candidate drivers for future experimental investigation. Applicant applied Malinois to 15,634,266 non-coding somatic mutations from the Catalogue of Somatic Mutations In Cancer (COSMIC) [41]. Applicant compared the number of observed mutations on promoters for Cancer Gene Census Hallmark (CGCH) genes against all other mutated promoters. Applicant found an enrichment of observed mutations in CGCH gene promoters in regions with increasing gene expression also enhanced activity in K562 (FIG. 6C). Furthermore, Applicant found that mutations with larger K562 allelic skew predictions were further enriched in CGCH gene promoters after controlling for high baseline predicted activity (FIG. 6D).

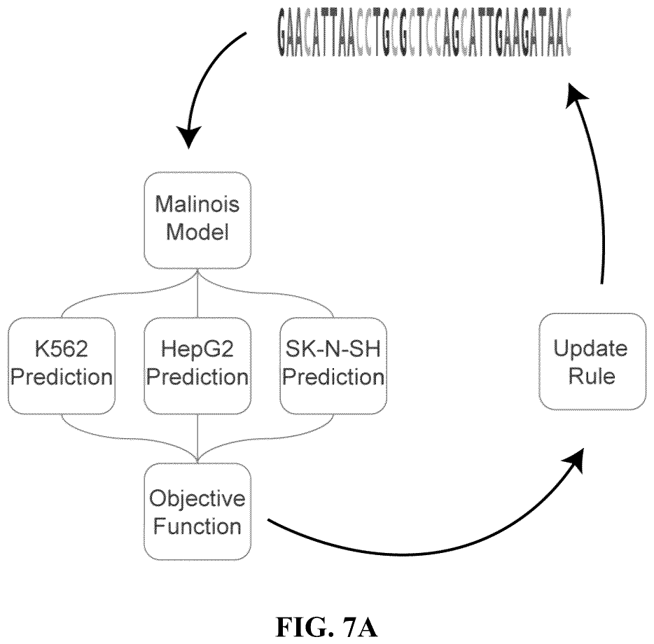

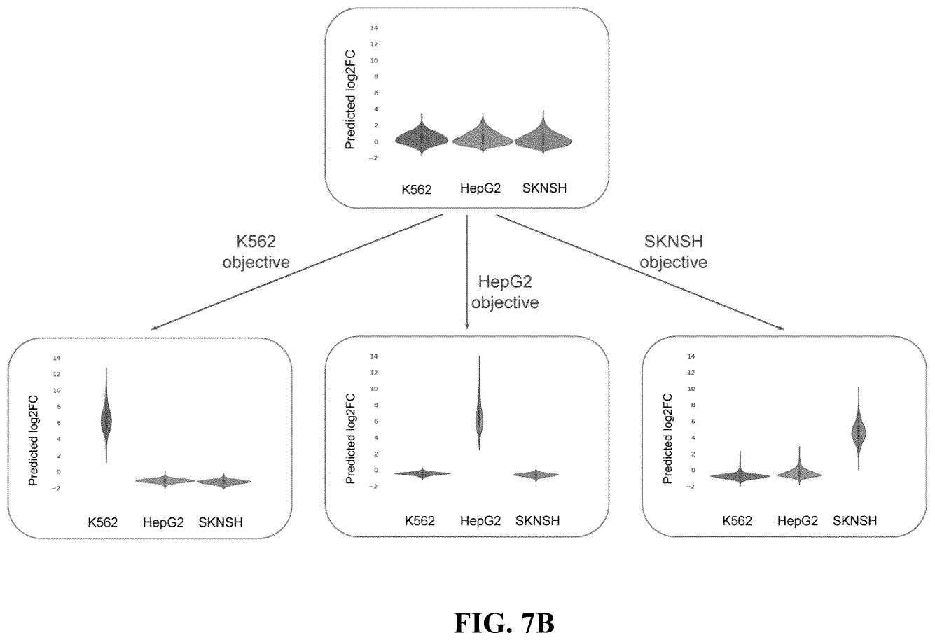

Malinois enables rational design of cell type specific enhancers. Finally, Applicant sought to rationally design synthetic sequences using Malinois. This will serve, in part, as the ultimate prospective validation experiment: capable of both exposing modeling pathologies and able test the credibility of extreme predictions. Applicant plugged Malinois into four sequence generation algorithms for rational sequence design: AdaLead [133], Fast SeqProp [92], simulated annealing [148], [11], and gradient based updates with random momentum (GURM described herein). These methods sequentially modify a starting sequence by computing a model prediction-based objective function and applying updates based on the result (FIG. 7A). The intention is to convert arbitrary sequences with uniform gene regulatory activity across K562, HepG2, and SK-N-SH to cell type specific (CTS) enhancers (FIG. 7B). Applicant generated 48,000 candidate sequences to drive CTS expression in each of three cell types using four generative algorithms. Applicant also extracted 12,000 naturally derived CTS sequences from the human genome using each DHS signal and Malinois predictions.

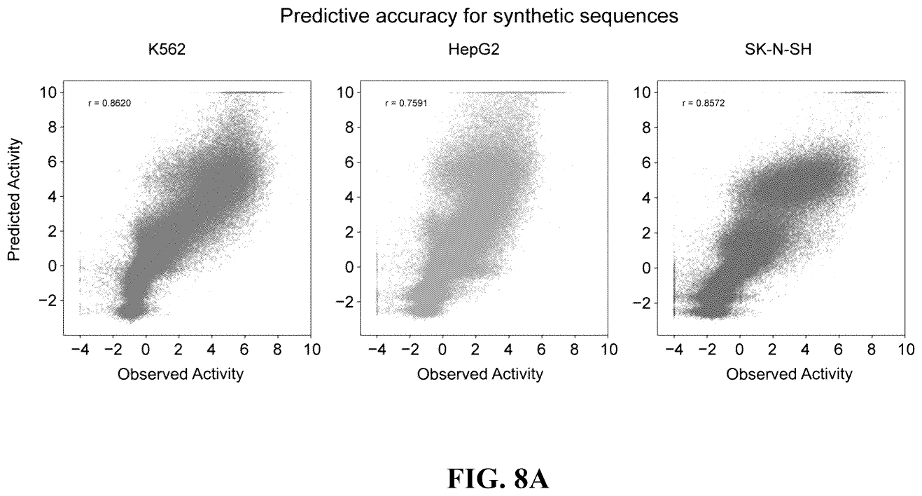

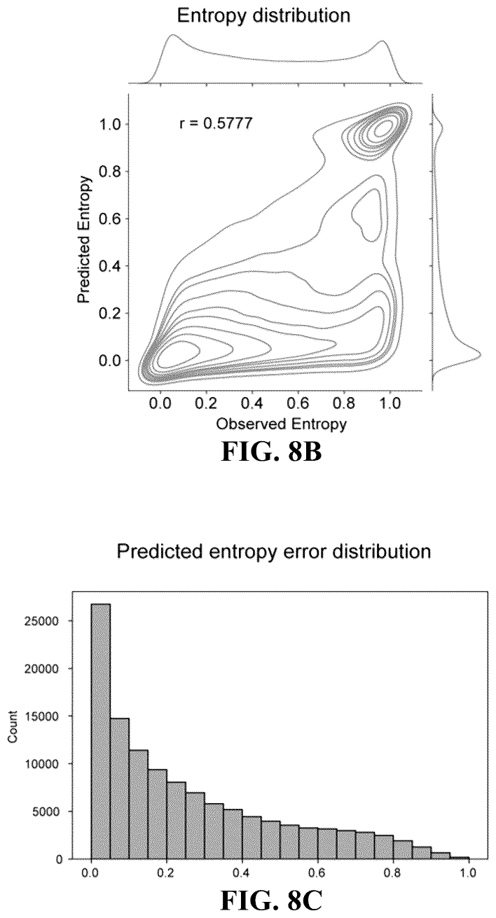

Next, Applicant performed an MPRA using this library in K562, HepG2, and SK-N-SH. Malinois pre-dictions were well correlated, and at similar levels to the initial test set, with the observed sequence activity in K562 (Pearson's r=0.86) and SK-N-SH (Pearson's r=0.85) (FIG. 8A). However, Applicant observed a substantial drop in prediction correlation for HepG2 (FIG. 8A, Pearson's r=0.76). To summarize CTS Applicant used entropy (H) of activity over 3 cell types:

$${p}_{i} = \frac{{e}^{{x}_{i}}}{{\sum }_{i} {e}^{{x}_{i}}} ,$$

$$H = - {\sum }_{i} {p}_{i} log {p}_{i} ,$$

where xᵢ corresponds to the predicted or measured MPRA activity in the i-th cell type. For this study 0≤H<1.1, and 0 indicates perfect cell type specificity. Using this metric, despite the drop in HepG2 accuracy, Malinois generally makes accurate predictions of entropy (FIG. 8B-8C).

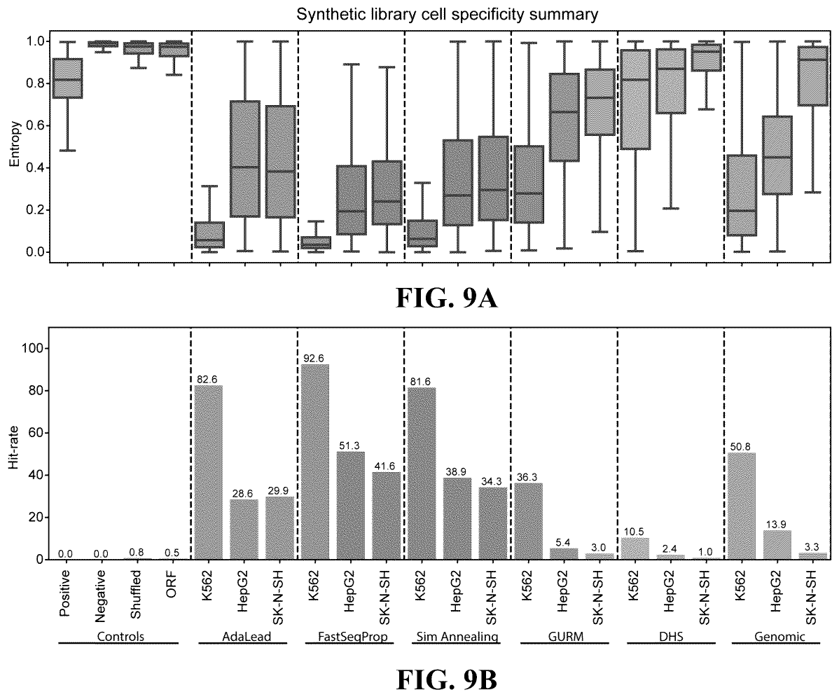

Overall, Applicant found that sequences selected based on Malinois predictions usually drive greater cell type specificity compared to sequences selected based on DHS signal (FIG. 9A). Furthermore, for 3 out of 4 generative algorithms, in silico designed sequences were on aggregate more specific than sequences chosen from the genome using Malinois. Applicant categorized sequences with H≤0.2 as CTS hits. Based on this cutoff, 3 generative algorithms produced CTS sequences at a far higher frequency than the genomic selection methods (FIG. 9B). Applicant's results indicate that deep learning models can reliably generate completely novel sequences that execute an intended function.

Discussion. The ability to quickly and accurately predict cis-regulatory function from DNA sequence alone would revolutionize Applicant's interpretation of genetic variation in humans. This would both aid Applicant's interpretation of loci associated with complex diseases and demystify the regulatory variation underpinning human evolution. Despite the prevalence of accurate chromatin state models based on vast troves of biochemical data, functional characterization models have languished due to relatively smaller data sets from a new class of still-evolving assays. In this study, Applicant has presented Malinois, a deep learning functional characterization model, trained on a comparably large and high-quality MPRA data set that was recently released in Phase 4 of ENCODE.

Malinois accurately reconstructs MPRA activity signal for three cell types, in silico enabling genome-wide extrapolation of MPRA. Applicant has shown genome-wide predictions are closely associated with biochemical markers of CRE identity and display similar resolution to DHS signal. Importantly, genome-wide MPRA predictions also correspond well with STARR-seq signal, a related functional characterization method that enables genome-wide analysis at lower resolution. Crucially, Applicant has shown Malinois identifies changes in CRE function induced by genetic variation found in humans. Thus, Applicant has shown deep learning models can rapidly expand the scope of insights gleaned from a targeted MPRA.

Deep learning models fit data remarkably well, including for genomics applications [77], [76], [125], [6]. However, this commonly leads to overfitting when models exploit spurious patterns in the training data, leading to poor generalizability for practical applications. In this study, Applicant tested the activity of synthetic sequences generated solely based on model predictions. Surprisingly, Applicant found Malinois accuracy remains mostly high for these artificially derived sequences. Most striking is the effective use of Fast SeqProp for sequence optimization. This method manipulates sequences by exploiting gradients calculated by Malinois to alter predicted activity. This is compelling; however, it can be confounded by model pathologies, and is similar to adversarial attacks by generative adversarial networks [57]. Further characterization of Applicant's model and results on synthetic sequences revealed the extent to which this affected Applicant's study. However, it remains that Applicant was able to effectively engineer a large number of cell type specific enhancer sequences ab initio. Overall, Applicant showed that MPRA can be used to train trust-worthy models that can utilized for biologically relevant applications.

Methods. Data. Applicant collected functional genomics data used in this study from the ENCODE portal [95]. This includes: MPRA analysis of UKBB/GTEx variants and the GATA1 locus (Tewhey Lab), STARR-seq of K562 (Reddy Lab), DHS signals (Stamatoyannopoulos and Crawford Labs), H3K27ac ChIP-seq (Bernstein Lab). Saturation mutagenesis MPRA was obtained from the Kircher Lab website [82].

Methods. Modeling. First, Applicant re-implemented Basset [77], a chromatin state classification model originally written in torch7, in PyTorch. This enabled Applicant to pre-train convolutional and linear layers on roughly 2 million DNA sites to predict DHS in 164 cell types per instruction at (github.com/davek44/Basset). Next, Applicant established a model selection framework that would allow us to test variable architectures which partially inherit weights from Applicant's PyTorch implementation of Basset. This framework makes two key modifications to Basset: (1) Applicant allowed a variable length stack of fully connected layers following the convolutional layers, and (2) Applicant added a variable length stack of branched linear layers which terminates at the output, with one dedicated branch per prediction task. While Applicant's final model architecture is substantially different from Basset, weights can be inherited prior to training when layers are the appropriate dimensions.

Applicant conducted hyperparameter optimization using the Google AI platform on the Google Cloud Platform. Applicant's final model with full architecture and hyperparameter specification can be accessed via a Google storage bucket//syrgoth/aip_ui_test/model_artifacts 20211113_021200 287348.tar.gz.

Sequence Generation. Applicant constructed a simple objective function to maximize predicted expression of a given sequence, x(s) in the ith cell type while reducing expression in the other j=i cells:

$${F}_{i} ( s ) = {x}_{i} ( s ) - {( max )}_{j \neq i} ( {x}_{j} ( s ) ) .$$

Applicant implemented four generation algorithms to propose DNA sequences that would maximize this function.

Fast SeqProp. Fast SeqProp (FSP) utilizes the straight though estimator [7] to optimize a distribution of sequences via gradient updates based on the output of a deep learning model. Applicant implemented FSP as described by Linder & Seelig except that Applicant excluded instance normalization, which impeded convergence in Applicant's hands.

AdaLead. Applicant implemented AdaLead, a simple genetic algorithm for black-box model-based sequence optimization as described by Sinai et al. [133].

Simulated Annealing. Applicant implemented simulated annealing (SA) based on Van Laarhoven & Aarts [148]. F, serves as the energy function when accepting proposals. Proposals were generated by first generating 1-3 random substitutions in the sequence. Proposals are accepted by a Metropolis-Hastings process where the energy of the system is tempered by Tₜ, temperature at a given iteration t. Tₜ is reduced exponentially to 0.

Gradient-based updates with random momentum. Applicant tried to implement a method that would provide a distribution of sequences based on the un-normalized probability distribution:

$$P ( s ) ∝ {e}^{{F}_{i} ( s )} .$$

To enable backpropagation to the inputs, Applicant reparameterized discrete nucleotide sequences using the Gumbel-Softmax trick [73]. Applicant then sampled reparameterized inputs using the No-U-Turn Sampler [68], from which Applicant in turn sampled discrete DNA sequences. Applicant calls this strategy gradient-based updates with random momentum (GURM).

Model-based selection from genomic sequences. Applicant scored the entire human genome (GRCh38) by applying Malinois to 200-nt windows using a 50-nt sliding window step size. Applicant selected the top sequences for the ith cell type based on Fᵢ.

DHS-based selection from genomics sequences. Applicant repeated the process used in Model-based selection from genomic sequences, except with DHS scores collected from the ENCODE portal [95].

MPRA using a synthetic sequence library. Design. Applicant generated 4000 sequence proposals to maximize cell type specific expression in each of K562, HepG2, and SK-N-SH, cells using each of the methods described in Sequence Generation (60000=4000 [oligos]×3 [cell types]×5 [algorithms]). Additionally, Applicant added ˜700 control sequences shared with the UKBB/GTEx library [134].

Assay. The proposal library was used to conduct an MPRA in K562, HepG2, and SK-N-SH using previously described methods [134], [141].

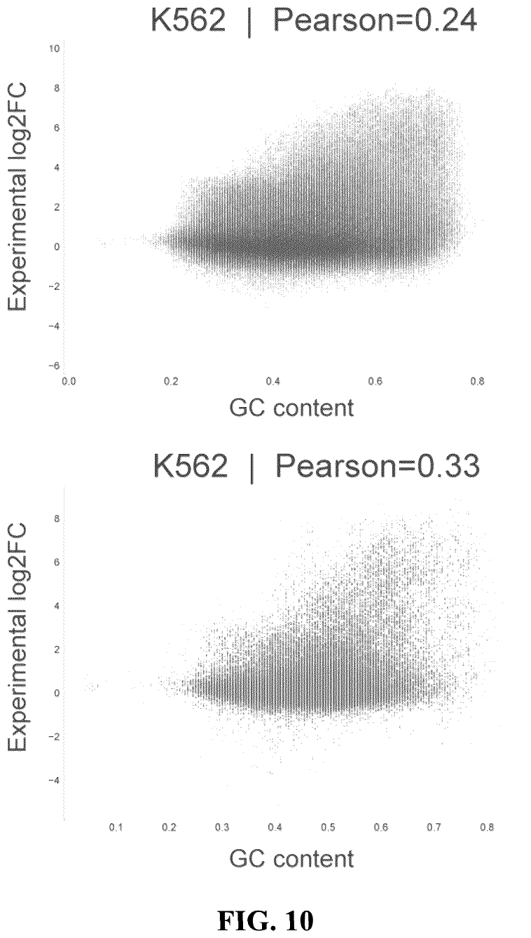

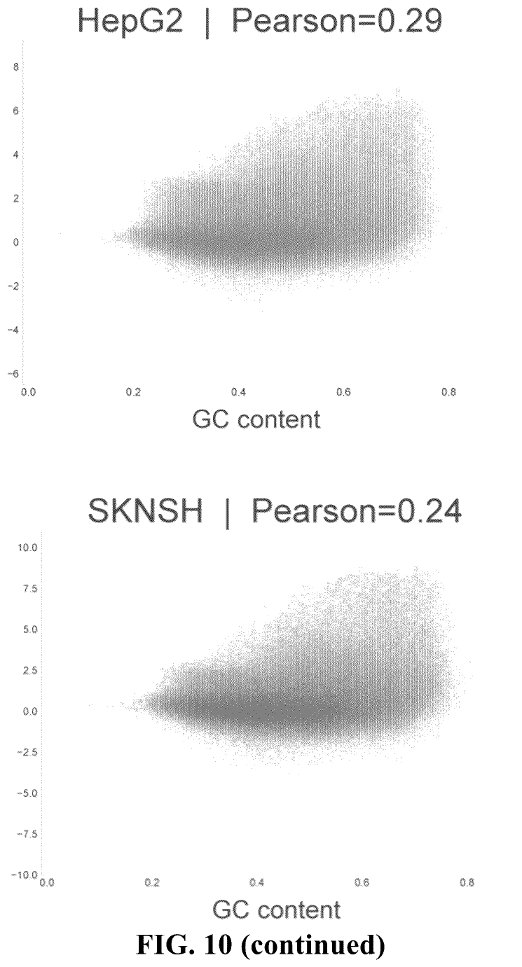

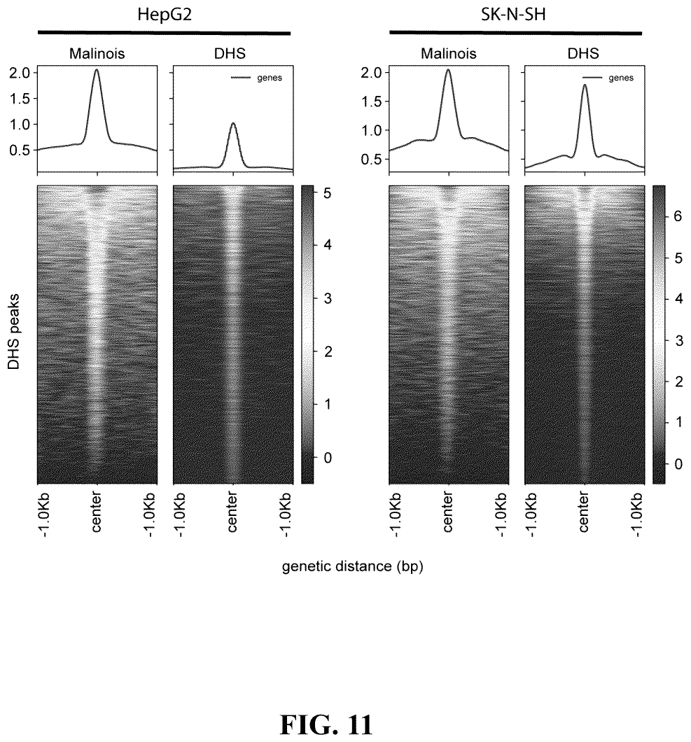

FIG. 10 shows the accuracy of GC content as a predictor of CRE activity in MPRA. (top row) GC analysis of test set [134]; (bottom) GC analysis of GATA1 tiling screen. FIG. 11 shows a comparison of Malinois predictions in HepG2 and SK-N-SH with DHS signal in the corresponding cell type 95.

### REFERENCES RELATED TO EXAMPLE 1

[1] 1000 Genomes Project Consortium, T. (2015). A global reference for human genetic varia-tion. Nature, 526 (7571), 68-74.
[5] Andersson, R., Gebhard, C., Miguel-Escalada, I., Hoof, I., Bornholdt, J., Boyd, M., Chen, Y., Zhao, X., Schmidl, C., Suzuki, T., Ntini, E., Arner, E., Valen, E., Li, K., Schwarzfischer, L., Glatz, D., Raithel, J., Lilje, B., Rapin, N., Bagger, F. O., Jørgensen, M., Andersen, P. R., Bertin, N., Rackham, O., Burroughs, A. M., Baillie, J. K., Ishizu, Y., Shimizu, Y., Furuhata, E., Maeda, S., Negishi, Y., Mungall, C. J., Meehan, T. F., Lassmann, T., Itoh, M., Kawaji, H., Kondo, N., Kawai, J., Lennartsson, A., Daub, C. O., Heutink, P., Hume, D. A., Jensen, T. H., Suzuki, H., Hayashizaki, Y., Müller, F., Forrest, A. R. R., Carninci, P., Rehli, M., Sandelin, A., & Consortium, T. F. (2014). An atlas of active enhancers across human cell types and tissues. Nature, 507 (7493), 455-461.
[6] Avsec, Z., Agarwal, V., Visentin, D., Ledsam, J. R., Grabska-Barwinska, A., Taylor, K. R., Assael, Y., Jumper, J., Kohli, P., & Kelley, D. R. (2021). Effective gene expression prediction from sequence by integrating long-range interactions. Nature Methods, 18 (10), 1196-1203.
[7] Bengio, Y., Leonard, N., & Courville, A. (2013). Estimating or propagating gradients through stochastic neurons for conditional computation. arXiv preprint ar Xiv: 1308.3432.
[11] Biswas, S., Kuznetsov, G., Ogden, P. J., Conway, N. J., Adams, R. P., & Church, G. M. (2018). Toward machine-guided design of proteins. BioRxiv.
[18] Campbell, P. J., Getz, G., Korbel, J. O., Stuart, J. M., Jennings, J. L., Stein, L. D., Perry, M. D., Nahal-Bose, H. K., Ouellette, B. F. F., Li, C. H., Rheinbay, E., Nielsen, G. P., Sgroi, D. C., Wu, C.-L., Faquin, W. C., Deshpande, V., Boutros, P. C., Lazar, A. J., Hoadley, K. A., Louis, D. N., Dursi, L. J., Yung, C. K., Bailey, M. H., Saksena, G., Raine, K. M., Buchhalter, I., Kleinheinz, K., Schlesner, M., Zhang, J., Wang, W., Wheeler, D. A., Ding, L., Simpson, J. T., O'Connor, B. D., Yakneen, S., Ellrott, K., Miyoshi, N., Butler, A. P., Royo, R., Shorser, S. I., Vazquez, M., Rausch, T., Tiao, G., Waszak, S. M., Rodriguez-Martin, B., Shringarpure, S., Wu, D. Y., Demidov, G. M., Delaneau, O., Hayashi, S., Imoto, S., Habermann, N., Segre, A. V., Garrison, E., Cafferkey, A., Alvarez, E. G., Heredia-Genestar, J. M., Muyas, F., Drech-sel, O., Bruzos, A. L., Temes, J., Zamora, J., Baez-Ortega, A., Kim, H.-L., Mashl, R. J., Ye, K., DiBiase, A., Huang, K. -l., Letunic, I., Mclellan, M. D., Newhouse, S. J., Shmaya, T., Kumar, S., Wedge, D. C., Wright, M. H., Yellapantula, V. D., Gerstein, M., Khurana, E., Marques-Bonet, T., Navarro, A., Bustamante, C. D., Siebert, R., Nakagawa, H., Easton, D. F., Ossowski, S., Tubio, J. M. C., De La Vega, F. M., Estivill, X., Yuen, D., Mihaiescu, G. L., Omberg, L., Ferretti, V., Sabarinathan, R., Pich, O., Gonzalez-Perez, A., Taylor-Weiner, A., Fittall, M. W., Demeulemeester, J., Tarabichi, M., Roberts, N. D., Van Loo, P., Cortés-Ciriano, I., Urban, L., Park, P., Zhu, B., Pitkänen, E., Li, Y., Saini, N., Klimczak, L. J., Weischenfeldt, J., Sidiropoulos, N., Alexandrov, L. B., Rabionet, R., Escaramis, G., Bosio, M., Holik, A. Z., Susak, H., Prasad, A., Erkek, S., Calabrese, C., Raeder, B., Harrington, E., Mayes, S., Turner, D., Juul, S., Roberts, S. A., Song, L., Koster, R., Mirabello, L., Hua, X., Tanskanen, T. J., Tojo, M., Chen, J., Aaltonen, L. A., Rätsch, G., Schwarz, R. F., Butte, A. J., Brazma, A., Chanock, S. J., Chatterjee, N., Stegle, O., Harismendy, O., Bova, G. S., Gor-denin, D. A., Haan, D., Sieverling, L., Feuerbach, L., Chalmers, D., Joly, Y., Knoppers, B., Molnár-Gabor, F., Phillips, M., Thorogood, A., Townend, D., Goldman, M., Fonseca, N. A., Xiang, Q., Craft, B., Piñeiro-Yáñez, E., Muñoz, A., Petryszak, R., Füllgrabe, A., Al-Shahrour, F., Keays, M., Haussler, D., Weinstein, J., Huber, W., Valencia, A., Papatheodorou, I., Zhu, J., Fan, Y., Torrents, D., Bieg, M., Chen, K., Chong, Z., Cibulskis, K., Eils, R., Fulton, R. S., Gelpi, J. L., Gonzalez, S., Gut, I. G., Hach, F., Heinold, M., Hu, T., Huang, V., Hutter, B., Jäger, N., Jung, J., Kumar, Y., Lalansingh, C., Leshchiner, I., Livitz, D., Ma, E. Z., Maruvka, Y. E., Milovanovic, A., Nielsen, M. M., Paramasivam, N., Pedersen, J. S., Puiggròs, M., Sahi-nalp, S. C., Sarrafi, I., Stewart, C., Stobbe, M. D., Wala, J. A., Wang, J., Wendl, M., Werner, J., Wu, Z., Xue, H., Yamaguchi, T. N., Yellapantula, V., Davis-Dusenbery, B. N., Grossman, R. L., Kim, Y., Heinold, M. C., Hinton, J., Jones, D. R., Menzies, A., Stebbings, L., Hess, J. M., Rosenberg, M., Dunford, A. J., Gupta, M., Imielinski, M., Meyerson, M., Beroukhim, R., Reimand, J., Dhingra, P., Favero, F., Dentro, S., Wintersinger, J., Rudneva, V., Park, J. W., Hong, E. P., Heo, S. G., Kahles, A., Lehmann, K.-V., Soulette, C. M., Shiraishi, Y., Liu, F., He, Y., Demircioğlu, D., Davidson, N. R., Greger, L., Li, S., Liu, D., Stark, S. G., Zhang, F., Amin, S. B., Bailey, P., Chateigner, A., Frenkel-Morgenstern, M., Hou, Y., Huska,M. R., Kilpinen, H., Lamaze, F. C., Li, C., Li, X., Li, X., Liu, X., Marin, M. G., Markowski, J., Nandi, T., Ojesina, A. I., Pan-Hammarström, Q., Park, P. J., Pedamallu, C. S., Su, H., Tan, P., Teh, B. T., Wang, J., Xiong, H., Ye, C., Yung, C., Zhang, X., Zheng, L., Zhu, S., Awadalla, P., Creighton, C. J., Wu, K., Yang, H., Göke, J., Zhang, Z., Brooks, A. N., Martin-corena, I., Rubio-Perez, C., Juul, M., Schumacher, S., Shapira, O., Tamborero, D., Mularoni, L., Hornshøj, H., Deu-Pons, J., Muiños, F., Bertl, J., Guo, Q., The ICGC/TCGA Pan-Cancer Analysis of Whole Genomes Consortium. Pan-cancer analysis of whole genomes. Nature (2020), 578 (7793), 82-93.
[35] Ernst, J., Melnikov, A., Zhang, X., Wang, L., Rogov, P., Mikkelsen, T. S., & Kellis, M. (2016). Genome-scale high-resolution mapping of activating and repressive nucleotides in regulatory regions. Nature Biotechnology, 34 (11), 1180-1190.
[41] Forbes, S. A., Beare, D., Boutselakis, H., Bamford, S., Bindal, N., Tate, J., Cole, C. G., Ward, S., Dawson, E., Ponting, L., Stefancsik, R., Harsha, B., Kok, C. Y., Jia, M., Jubb, H., Sondka, Z., Thompson, S., De, T., & Campbell, P. J. (2016). COSMIC: somatic cancer genetics at high-resolution. Nucleic Acids Research, 45 (D1), D777-D783.
[42] Fraser, H. B. (2013). Gene expression drives local adaptation in humans. Genome research, 23 (7), 1089-1096.
[43] Fulco, C. P., Munschauer, M., Anyoha, R., Munson, G., Grossman, S. R., Perez, E. M., Kane, M., Cleary, B., Lander, E. S., & Engreitz, J. M. (2016). Systematic mapping of func-tional enhancer-promoter connections with CRISPR interference. Science, (pp. aag2445).
[44] Fulco, C. P., Nasser, J., Jones, T. R., Munson, G., Bergman, D. T., Subramanian, V., Gross-man, S. R., Anyoha, R., Doughty, B. R., Patwardhan, T. A., Nguyen, T. H., Kane, M., Perez, E. M., Durand, N. C., Lareau, C. A., Stamenova, E. K., Aiden, E. L., Lander, E. S., & En-greitz, J. M. (2019). Activity-by-contact model of enhancer-promoter regulation from thousands of crispr perturbations. Nature Genetics, 51 (12), 1664-1669.
[49] Genereux, D. P., Serres, A., Armstrong, J., Johnson, J., Marinescu, V. D., Muren, E., Juan, D., Bejerano, G., Casewell, N. R., Chemnick, L. G., Damas, J., Di Palma, F., Diekhans, M., Fiddes, I. T., Garber, M., Gladyshev, V. N., Goodman, L., Haerty, W., Houck, M. L., Hubley, R., Kivioja, T., Koepfli, K.-P., Kuderna, L. F. K., Lander, E. S., Meadows, J. R. S., Murphy, W. J., Nash, W., Noh, H. J., Nweeia, M., Pfenning, A. R., Pollard, K. S., Ray, D. A., Shapiro, B., Smit, A. F. A., Springer, M. S., Steiner, C. C., Swofford, R., Taipale, J., Teeling, E. C., Turner-Maier, J., Alfoldi, J., Birren, B., Ryder, O. A., Lewin, H. A., Paten, B., Marques-Bonet, T., Lindblad-Toh, K., Karlsson, E. K., & Consortium, Z. (2020). A comparative genomics multitool for scientific discovery and conservation. Nature, 587 (7833), 240-245.
[52] Ghandi, M., Mohammad-Noori, M., Ghareghani, N., Lee, D., Garraway, L., & Beer, M. A. (2016). gkmSVM: an R package for gapped-kmer SVM. Bioinformatics, 32 (14), 2205-2207.
[57] Goodfellow, I., Pouget-Abadie, J., Mirza, M., Xu, B., Warde-Farley, D., Ozair, S., Courville, A., & Bengio, Y. (2014). Generative adversarial nets. In Z. Ghahramani, M. Welling, C. Cortes, N. Lawrence, & K. Weinberger (Eds.), Advances in Neural Information Processing Systems, volume 27: Curran Associates, Inc.
[61] GTEx Consortium (2015). Human genomics. the Genotype-Tissue expression (GTEx) pilot analysis: multitissue gene regulation in humans. Science, 348 (6235), 648-660.
[62] GTEx Consortium, Laboratory, Data Analysis &Coordinating Center (LDACC)—Analysis Working Group, Statistical Methods groups-Analysis Working Group, Enhancing GTEx (eGTEx) groups, NIH Common Fund, NIH/NCI, NIH/NHGRI, NIH/NIMH, NIH/NIDA, Biospecimen Collection Source Site-NDRI, Biospecimen Collection Source Site—RPCI, Biospecimen Core Resource—VARI, Brain Bank Repository—University of Miami Brain Endowment Bank, Leidos Biomedical-Project Management, ELSI Study, Genome Browser Data Integration & Visualization—EBI, Genome Browser Data Integration & Visualization—UCSC Genomics Institute, University of California Santa Cruz, Lead analysts: Laboratory, Data Analysis &Coordinating Center (LDACC): NIH program management: Biospecimen collection: Pathology: eQTL manuscript working group: Battle, A., Brown, C. D., Engelhardt, B. E., & Montgomery, S. B. (2017). Genetic effects on gene expression across human tissues. Nature, 550 (7675), 204-213.
[64] Hardison, R. C. & Taylor, J. (2012). Genomic approaches towards finding cis-regulatory modules in animals. Nature Reviews Genetics, 13 (7), 469-483.
[67] Hindorff, L. A., Sethupathy, P., Junkins, H. A., Ramos, E. M., Mehta, J. P., Collins, F. S., & Manolio, T. A. (2009). Potential etiologic and functional implications of genome-wide association loci for human diseases and traits. Proceedings of the National Academy of Sciences, 106 (23), 9362-9367.
[68] Hoffman, M. D. & Gelman, A. (2014). The no-u-turn sampler: Adaptively setting path lengths in hamiltonian monte carlo. Journal of Machine Learning Research, 15 (47), 1593-1623.
[73] Jang, E., Gu, S., & Poole, B. (2017). Categorical reparameterization with gumbel-softmax. In International Conference on Learning Representations.
[75] Karczewski, K. J., Francioli, L. C., Tiao, G., Cummings, B. B., Alföldi, J., Wang, Q., Collins, R. L., Laricchia, K. M., Ganna, A., Birnbaum, D. P., Gauthier, L. D., Brand, H., Solomon-son, M., Watts, N. A., Rhodes, D., Singer-Berk, M., England, E. M., Seaby, E. G., Kosmicki, J. A., Walters, R. K., Tashman, K., Farjoun, Y., Banks, E., Poterba, T., Wang, A., Seed, C., Whiffin, N., Chong, J. X., Samocha, K. E., Pierce-Hoffman, E., Zappala, Z., O'Donnell-Luria, A. H., Minikel, E. V., Weisburd, B., Lek, M., Ware, J. S., Vittal, C., Armean, I. M., Bergelson, L., Cibulskis, K., Connolly, K. M., Covarrubias, M., Donnelly, S., Ferriera, S., Gabriel, S., Gentry, J., Gupta, N., Jeandet, T., Kaplan, D., Llanwarne, C., Munshi, R., Novod, S., Petrillo, N., Roazen, D., Ruano-Rubio, V., Saltzman, A., Schleicher, M., Soto, J., Tibbetts, K., Tolonen, C., Wade, G., Talkowski, M. E., Aguilar Salinas, C. A., Ahmad, T., Albert, C. M., Ardissino, D., Atzmon, G., Barnard, J., Beaugerie, L., Benjamin, E. J., Boehnke, M., Bonnycastle, L. L., Bottinger, E. P., Bowden, D. W., Bown, M. J., Cham-bers, J. C., Chan, J. C., Chasman, D., Cho, J., Chung, M. K., Cohen, B., Correa, A., Dabelea, D., Daly, M. J., Darbar, D., Duggirala, R., Dupuis, J., Ellinor, P. T., Elosua, R., Erdmann, J., Esko, T., Färkkilä, M., Florez, J., Franke, A., Getz, G., Glaser, B., Glatt, S. J., Gold-stein, D., Gonzalez, C., Groop, L., Haiman, C., Hanis, C., Harms, M., Hiltunen, M., Holi, M. M., Hultman, C. M., Kallela, M., Kaprio, J., Kathiresan, S., Kim, B.-J., Kim, Y. J., Kirov, G., Kooner, J., Koskinen, S., Krumholz, H. M., Kugathasan, S., Kwak, S. H., Laakso, M., Lehtimäki, T., Loos, R. J. F., Lubitz, S. A., Ma, R. C. W., MacArthur, D. G., Marrugat, J., Mattila, K. M., McCarroll, S., Mccarthy, M. I., McGovern, D., McPherson, R., Meigs, J. B., Melander, O., Metspalu, A., Neale, B. M., Nilsson, P. M., O'Donovan, M. C., Ongur, D., Orozco, L., Owen, M. J., Palmer, C. N. A., Palotie, A., Park, K. S., Pato, C., Pulver, A. E., Rahman, N., Remes, A. M., Rioux, J. D., Ripatti, S., Roden, D. M., Saleheen, D., Salomaa, V., Samani, N. J., Scharf, J., Schunkert, H., Shoemaker, M. B., Sklar, P., Soininen, H., Sokol, H., Spector, T., Sullivan, P. F., Suvisaari, J., Tai, E. S., Teo, Y. Y., Tiinamaija, T., Tsuang, M., Turner, D., Tusie-Luna, T., Vartiainen, E., Vawter, M. P., Watkins, H., Weersma, R. K., Wessman, M., Wilson, J. G., Xavier, R. J., & Consortium, G. A. D. (2020). The mutational constraint spectrum quantified from variation in 141,456 humans. Nature, 581 (7809), 434-443.
[76] Kelley, D. R., Reshef, Y. A., Belanger, D., McLean, C., Snoek, J., & Bileschi, M. (2018). Se-quential regulatory activity prediction across chromosomes with convolutional neural networks. bioRxiv, (pp. 161851).
[77] Kelley, D. R., Snoek, J., & Rinn, J. L. (2016). Basset: Learning the regulatory code of the accessible genome with deep convolutional neural networks. Genome Research, 26 (7), 990-999.
Kheradpour, P., Ernst, J., Melnikov, A., Rogov, P., Wang, L., Zhang, X., Alston, J., Mikkelsen, T. S., & Kellis, M. (2013). Systematic dissection of regulatory motifs in 2000 predicted human enhancers using a massively parallel reporter assay. Genome research, 23 (5), 800-811.
Kircher, M., Xiong, C., Martin, B., Schubach, M., Inoue, F., Bell, R. J. A., Costello, J. F., Shendure, J., & Ahituv, N. (2019). Saturation mutagenesis of twenty disease-associated regulatory elements at single base-pair resolution. Nature Communications, 10 (1), 3583.
[87] Lee, D., Gorkin, D. U., Baker, M., Strober, B. J., Asoni, A. L., McCallion, A. S., & Beer, M. A. (2015). A method to predict the impact of regulatory variants from DNA sequence. Nature Genetics, 47 (8), 955-961.
[89] LeProust, E. M., Peck, B. J., Spirin, K., McCuen, H. B., Moore, B., Namsaraev, E., & Caruthers, M. H. (2010). Synthesis of high-quality libraries of long (150mer) oligonucleotides by a novel depurination controlled process. Nucleic acids research, 38 (8), 2522-2540.
[92] Linder, J. & Seelig, G. (2021). Fast activation maximization for molecular sequence design. BMC Bioinformatics, 22 (1), 510.
[95] Long, H. K., Prescott, S. L., & Wysocka, J. (2016). Ever-changing landscapes: Transcriptional enhancers in development and evolution. Cell, 167 (5), 1170-1187.
[100] Luo, Y., Hitz, B. C., Gabdank, I., Hilton, J. A., Kagda, M. S., Lam, B., Myers, Z., Sud, P., Jou, J., Lin, K., Baymuradov, U. K., Graham, K., Litton, C., Miyasato, S. R., Strattan, J. S., Jolanki, O., Lee, J.-W., Tanaka, F. Y., Adenekan, P., O'Neill, E., & Cherry, J. M. (2019). New developments on the Encyclopedia of DNA Elements (ENCODE) data portal. Nucleic Acids Research, 48 (D1), D882-D889.
[102] Melnikov, A., Murugan, A., Zhang, X., Tesileanu, T., Wang, L., Rogov, P., Feizi, S., Gnirke, A., Callan, C. G., Kinney, J. B., Kellis, M., Lander, E. S., & Mikkelsen, T. S. (2012). Sys-tematic dissection and optimization of inducible enhancers in human cells using a massively parallel reporter assay. Nature Biotechnology, 30 (3), 271-277.
[102] Movva, R., Greenside, P., Marinov, G. K., Nair, S., Shrikumar, A., & Kundaje, A. (2019). Deciphering regulatory dna sequences and noncoding genetic variants using neural network models of massively parallel reporter assays. PLOS ONE, 14 (6), 1-20.
[113] Quang, D. & Xie, X. (2016). DanQ: A hybrid convolutional and recurrent deep neural network for quantifying the function of DNA sequences. Nucleic Acids Research, 44 (11), e107-e107.
[115] Ramirez, F., Ryan, D. P., Grüning, B., Bhardwaj, V., Kilpert, F., Richter, A. S., Heyne, S., Dündar, F., & Manke, T. (2016). deepTools2: a next generation web server for deep-sequencing data analysis. Nucleic Acids Research, 44 (W1), W160-W165.
[118] Reilly, S. K., Gosai, S. J., Gutierrez, A., Mackay-Smith, A., Ulirsch, J. C., Kanai, M., Mouri, K., Berenzy, D., Kales, S., Butler, G. M., Gladden-Young, A., Bhuiyan, R. M., Stitzel, M. L., Finucane, H. K., Sabeti, P. C., & Tewhey, R. (2021). Direct characterization of cis-regulatory elements and functional dissection of complex genetic associations using hcr-flowfish. Na-ture 1166-1176. Genetics, 53 (8),
[120] Rheinbay, E., Nielsen, M. M., Abascal, F., Wala, J. A., Shapira, O., Tiao, G., Hornshøj, H., Hess, J. M., Juul, R. I., Lin, Z., Feuerbach, L., Sabarinathan, R., Madsen, T., Kim, J., Mularoni, L., Shuai, S., Lanzós, A., Herrmann, C., Maruvka, Y. E., Shen, C., Amin, S. B., Ban-dopadhayay, P., Bertl, J., Boroevich, K. A., Busanovich, J., Carlevaro-Fita, J., Chakravarty, D., Chan, C. W. Y., Craft, D., Dhingra, P., Diamanti, K., Fonseca, N. A., Gonzalez-Perez, A., Guo, Q., Hamilton, M. P., Haradhvala, N. J., Hong, C., Isaev, K., Johnson, T. A., Juul, M., Kahles, A., Kahraman, A., Kim, Y., Komorowski, J., Kumar, K., Kumar, S., Lee, D., Lehmann, K.-V., Li, Y., Liu, E. M., Lochovsky, L., Park, K., Pich, O., Roberts, N. D., Sak-sena, G., Schumacher, S. E., Sidiropoulos, N., Sieverling, L., Sinnott-Armstrong, N., Stew-art, C., Tamborero, D., Tubio, J. M. C., Umer, H. M., Uuskula-Reimand, L., Wadelius, C., Wadi, L., Yao, X., Zhang, C.-Z., Zhang, J., Haber, J. E., Hobolth, A., Imielinski, M., Kellis, M., Lawrence, M. S., von Mering, C., Nakagawa, H., Raphael, B. J., Rubin, M. A., Sander, C., Stein, L. D., Stuart, J. M., Tsunoda, T., Wheeler, D. A., Johnson, R., Reimand, J., Gerstein, M., Khurana, E., Campbell, P. J., López-Bigas, N., Bader, G. D., Barenboim, J., Beroukhim, R., Brunak, S., Chen, K., Choi, J. K., Deu-Pons, J., Fink, J. L., Frigola, J., Gambacorti-Passerini, C., Garsed, D. W., Getz, G., Gut, I. G., Haan, D., Harmanci, A. O., Helmy, M., Hodzic, E., Izarzugaza, J. M. G., Kim, J. K., Korbel, J. O., Larsson, E., Li, S., Li, X., Lou, S., Marchal, K., Martincorena, I., Martinez-Fundichely, A., McGillivray, P. D., Mey-erson, W., Muiños, F., Paczkowska, M., Park, K., Pedersen, J. S., Pons, T., Pulido-Tamayo, S., Reyes-Salazar, I., Reyna, M. A., Rubio-Perez, C., Sahinalp, S. C., Salichos, L., Shackleton, M., Shrestha, R., Valencia, A., Vazquez, M., Verbeke, L. P. C., Wang, J., Warrell, J., Waszak, S. M., Weischenfeldt, J., Wu, G., Yu, J., Zhang, X., Zhang, Y., Zhao, Z., Zou, L., Akdemir, K. C., Alvarez, E. G., Baez-Ortega, A., Boutros, P. C., Bowtell, D. D. L., Brors, B., Burns, K. H., Chan, K., Cortes-Ciriano, I., Dueso-Barroso, A., Dunford, A. J., Edwards, P. A., Estivill, X., Etemadmoghadam, D., Frenkel-Morgenstern, M., Gordenin, D. A., Hutter, B., Jones, D. T. W., Ju, Y. S., Kazanov, M. D., Klimczak, L. J., Koh, Y., Lee, E. A., Lee, J. J.-K., Lynch, A. G., Macintyre, G., Markowetz, F., Meyerson, M., Miyano, S., Navarro, F. C. P., Ossowski, S., Park, P. J., Pearson, J. V., Puiggròs, M., Rippe, K., Roberts, S. A., Rodriguez-Martin, B., Scully, R., Torrents, D., Villasante, I., Waddell, N., Yang, L., Yoon, S.-S., Zamora, J., Drivers, P. C. A. W. G., Group, F. I. W., Group, P. S. V. W., & Consortium, P. C. A. W. G. (2020). Analyses of non-coding somatic drivers in 2,658 cancer whole genomes. Na-ture, 578 (7793), 102-111.
[125] Sample, P. J., Wang, B., Reid, D. W., Presnyak, V., McFadyen, I. J., Morris, D. R., & Seelig, G. (2019). Human 5′UTR design and variant effect prediction from a massively parallel translation assay. Nature Biotechnology, 37 (7), 803-809.
[130] Shigaki, D., Adato, O., Adhikari, A. N., Dong, S., Hawkins-Hooker, A., Inoue, F., Juven-Gershon, T., Kenlay, H., Martin, B., Patra, A., Penzar, D. D., Schubach, M., Xiong, C., Yan, Z., Boyle, A. P., Kreimer, A., Kulakovskiy, I. V., Reid, J., Unger, R., Yosef, N., Shendure, J., Ahituv, N., Kircher, M., & Beer, M. A. (2019). Integration of multiple epigenomic marks improves prediction of variant impact in saturation mutagenesis reporter assay. Human Mutation, 40 (9), 1280-1291.
[133] Sinai, S., Wang, R., Whatley, A., Slocum, S., Locane, E., & Kelsic, E. D. (2020). Adalead: A simple and robust adaptive greedy search algorithm for sequence design.
[134] Siraj, L., Ulirsch, J., Dewey, H., Kales, S., Kanai, M., Berenzy, D., Mouri, K., Reilly, S., Fin-ucane, H., & Tewhey, R. (2022). Quantifying the functional effects of 234,448 likely causal regulatory variants underlying complex human traits. in preparation.
[135] Snoek, J., Larochelle, H., & Adams, R. P. (2012). Practical bayesian optimization of machine learning algorithms. In F. Pereira, C. Burges, L. Bottou, & K. Weinberger (Eds.), Advances in Neural Information Processing Systems, volume 25: Curran Associates, Inc.
[136] Sondka, Z., Bamford, S., Cole, C. G., Ward, S. A., Dunham, I., & Forbes, S. A. (2018). The cosmic cancer gene census: describing genetic dysfunction across all human cancers. Nature Reviews Cancer, 18 (11), 696-705.
[141] Tewhey, R., Kotliar, D., Park, D. S., Liu, B., Winnicki, S., Reilly, S. K., Andersen, K. G., Mikkelsen, T. S., Lander, E. S., Schaffner, S. F., & Sabeti, P. C. (2016). Direct Identification of Hundreds of Expression-Modulating Variants using a Multiplexed Reporter Assay. Cell, 165 (6), 1519-1529.
[147] Ulirsch, J. C., Nandakumar, S. K., Wang, L., Giani, F. C., Zhang, X., Rogov, P., Melnikov, A., McDonel, P., Do, R., Mikkelsen, T. S., & Sankaran, V. G. (2016). Systematic functional dissection of common genetic variation affecting red blood cell traits. Cell, 165 (6), 1530-1545.
[148] Van Laarhoven, P. J. & Aarts, E. H. (1987). Simulated annealing. In Simulated annealing: Theory and applications (pp. 7-15). Springer.
[159] Wittkopp, P. J. & Kalay, G. (2012). Cis-regulatory elements: molecular mechanisms and evolutionary processes underlying divergence. Nature Reviews Genetics, 13 (1), 59-69.
[163] Zhou, J. & Troyanskaya, O. G. (2015). Predicting effects of noncoding variants with deep learning-based sequence model. Nature Methods, 12 (10), 931-934.
[164] Zou, J., Huss, M., Abid, A., Mohammadi, P., Torkamani, A., & Telenti, A. (2019). A primer on deep learning in genomics. Nature Genetics, 51 (1), 12-18.

### Example 2—Functional Prediction and Machine-Guided Design of Cis-Regulatory Elements from Massively Parallel Reporter Assays

Biological sequence models accurately learn the logic underlying cis-regulatory elements (CREs) and have many promising applications in medicine and biotechnology. Here, Applicant combines Malinois, a convolutional neural network that predicts CRE function based on massively parallel reporter assays (MPRAs) in three cell types, with several algorithms for biological sequence design (Fast SeqProp, Simulated Annealing, and AdaLead) to engineer thousands of synthetic CREs with cell-type-specific regulatory activity. Applicant showed by MPRA that the vast majority of designed sequences from all three design algorithms confer the expected CRE activity. These sequences employ novel combinations of transcription factor binding motifs to simultaneously increase gene expression in one cell type while reducing expression in others. As such, synthetic sequences can achieve higher cell-type-specific regulatory activity than any natural sequences we tested. Finally, we selected two synthetic neuron-specific CREs to drive the expression of an integrated LacZ transgene in mice. One of these sequences reliably drives expression in the brains of 15-day-old mouse embryos. This work provides a generalizable approach to rationally design CREs that can jointly refine transgene expression across several cell types.

### Example 3—Genome-Wide, Nucleotide-Resolution Maps of Cis-Regulatory Element Function Using Deep-Learning Models of Massively Parallel Reporter Assays

Comprehensively quantifying the gene-regulatory potential of DNA remains a challenge in genomics limiting our understanding of regulatory grammar. A Massively Parallel Reporter Assay (MPRA) is a high-throughput functional genomic experimental platform that directly measures the activity of cis-regulatory elements (CREs) with the sensitivity to identify single-nucleotide variants that modulate regulatory activity. However, applying this in-vitro framework to provide nucleotide-resolution dissection of CRE function genome-wide is intractable. To circumvent this constraint, Applicant developed Malinois, a convolutional neural network with independent task-specific linear layers trained to predict the cis-regulatory activity of DNA sequences in three cell types using high-quality MPRA data. Malinois accurately reproduces reporter assays (minimum Pearson's r=0.87), as well as tiling and saturation mutagenesis screens, and is well associated with chromatin accessibility and H3K27ac signals. Leveraging Malinois, Applicant constructed a genome-wide track of single-nucleotide contribution scores for each prediction task by using Sampled Integrated Gradients, a novel adaptation of the feature attribution method Integrated Gradients that efficiently approximates the linearly-interpolated gradients over discrete-input spaces avoiding non-one-hot input evaluations and averaging gradients sampled from the path to the background distribution. This work provides an unprecedented dataset that extrapolates the MPRA cis-regulatory signal in three cell types to the whole human genome at a nucleotide level, advancing our means for investigating regulatory grammar.

### Example 4

### Introduction

Since the completion of the human genome, a major goal of genomics has been to achieve literacy of the genome. This includes the 98% of the-genome that does not code for protein-coding genes and instead controls the temporal and cell-specific expression of genes. Major efforts have sought to define the ‘regulatory grammar’ and the logical rules underlying how cis-regulatory elements (CREs) impart biochemical function on gene expression. CRE activity arises through the combinatorial action of transcription factor (TF) binding, genome looping, epigenetic modifications, and more, all of which can be directed by features encoded in the genetic sequence. The regulatory grammar conferring cell-type specific activity is thought to arise through the higher order semantic and syntactic combinations of activating and repressing TF vocabularies, however, this combinatorial logic has not been fully solved.

The ability to engineer CREs with specified function ab initio would be a display of regulatory code literacy with biotechnology and clinical applications. Designed, highly precise, cell-type specific transcriptional control would find use in specialized reporters, medicinal transgenes, and gene therapies, but has been largely elusive at scale for most tissues. Millions of putative CREs with diverse patterns of tissue-activity have been discovered and used over the past decade yet pleiotropic expression remains a major obstacle limiting their utility for clinical applications¹. Furthermore, the reservoir of potential CRE sequences in our genome and the selection constraints that shape them may not match desired expression objectives. Our ability to design CRE sequences with cell-type specific activity is currently limited in three areas: 1) accurate regulatory grammar models of how genetic sequences lead to CRE activity, 2) precision of such models across cell types, and 3) the ability to efficiently search and validate a large search space, as a 200-bp nucleotide can encodes 2.58×10\hat{ }120 distinct sequences.

Recent advancements in both measuring and modeling CREs have allowed us to overcome barriers to design. First, deep learning has recently emerged as an effective tool to accurately model the relationship between genetic sequences and biological features by exploiting large data sets²⁻⁸. Convolutional neural networks in particular have been highly effective for modeling diverse epigenomic signatures in many different cells and tissues from DNA sequence. While these sequence models are promising tools to interpret genetic sequences^{5, 6, 9}, they have largely been trained off of, and predict epigenomic signatures rather than CRE activity.

Secondly, massively parallel reporter assays (MPRAs) have become a powerful approach to directly characterize cis-regulatory activity potential for thousands of sequences simultaneously and across cell types. This technology has been used to functionally characterize hundreds of thousands of CREs in a programmable fashion; and such data has been shown to serve as a valuable training set on which to train models of CRE activity, extract regulatory syntax, and provide insights into transcriptional specificity. Computational models of CRE function, while millions of times faster than experimentation, are still only capable of characterizing a fraction of possible CRE sequences. Therefore, when designing new elements, it is essential to efficiently explore the candidate sequence space.

This example at least demonstrates a successful method to engineer novel synthetic CREs which Applicant used to create CREs that are capable of driving gene expression with highly cell-type specificity. Applicant achieved this by leveraging innovations in modeling regulatory grammar across cell types, efficient sequence space searching, and an experimental system that can validate thousands of CREs in parallel. Using a recently generated database of uniformly processed MPRA experiments which characterized an unprecedented number of CREs, we train an accurate deep-learning model that can rapidly predict activity for any sequence in silico. Coupled to sequence generation algorithms, we deploy our model to generate thousands of cell-type specific, synthetic CREs, which Applicant functionally validate using MPRAs. Together Applicant provides a generalizable framework to prospectively engineer CREs and demonstrate an ability to “write” regulatory code that has desired function across vertebrates in-vivo.

### Results

### Deep Learning can Accurately Model Cis-Regulatory Activity of DNA

Applicant endeavored to design an accurate model of regulatory DNA sequence function specifically tailored to predict cis-regulatory element (CRE) activity, rather than indirect epigenetic correlates. Applicant chose to train on model on the regulatory output of 776,475 200 nucleotide sequences assayed by MPRA, which directly measures CRE activity. These MPRAs were conducted by a single lab using consistent experimental and analytical pipelines. In total, Applicant collected functional CRE measurements from 67,480,007 bp of sequence derived from the genome in three cell types K562, HepG2, and SK-N-SH (FIG. 1A, left side).

Applicant's model, Malinois, was trained on this data in order to enable in silico, cell-type informed CRE activity of any arbitrary sequence. Applicant constructed a model, which framed this as a multi-task regression problem using fixed length, one-hot inputs. See also Example 1. Prior attempts to model functional characterization of CRE activity using deep learning were limited by small data sets which tested relatively few independent elements in the genome.

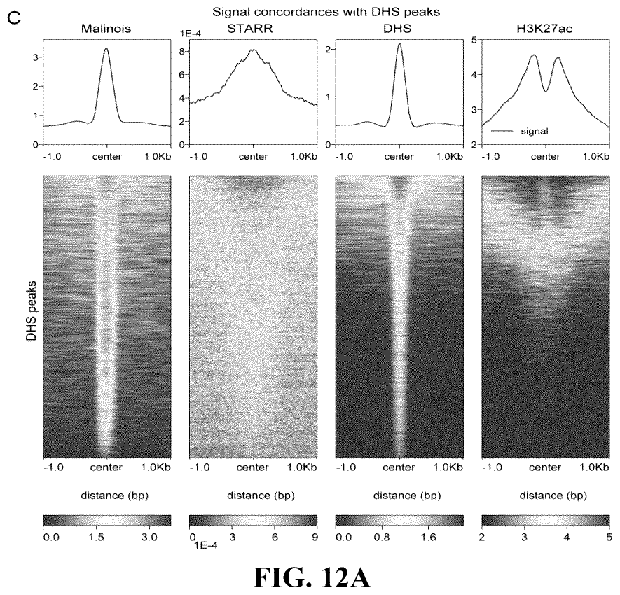

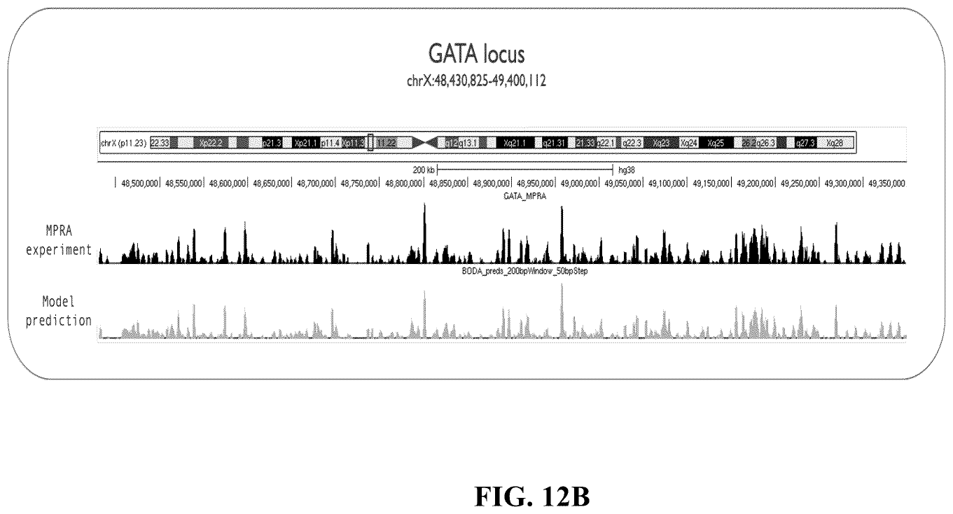

Malinois accurately predicts MPRA activity across cell types and successfully recapitulates biologically meaningful regulatory potential of genomic loci. For sequences held out from training, Malinois predictions in K562, HepG2, and SK-N-SH are highly correlated with empirical measurements (FIG. 1B; Pearson's r≥0.88; Spearman's p ≥0.81), and demonstrated cell specificity on par with experimental results. In other words, pairwise cell-type signal/prediction analysis and fraction correctly identified sequence as cell specific. In addition, we observed a strong correlation (Pearson's r=0.91) with predictions made for K562 in an orthogonal MPRA study that comprehensively tested all sequences from a 1 Mb window encompassing GATA1 (FIG. 12B).

Given Malinois can accurately model MPRA activity, we investigated the correspondence between a genome-wide prediction map and orthogonal approaches for characterizing CREs. Applicant found that Malinois predictions of activity in K562 are significantly associated with CREs determined by genome-wide functional characterization (STARR-seq) and candidate CREs identified by active chromatin maps (DHS-seq and H3K27ac ChIP-seq) (FIG. 12A). This gives us confidence that functional sequences identified as active by Malinois correspond to known endogenous measures of CRE while providing a more direct biochemical readout of transcriptional activity.

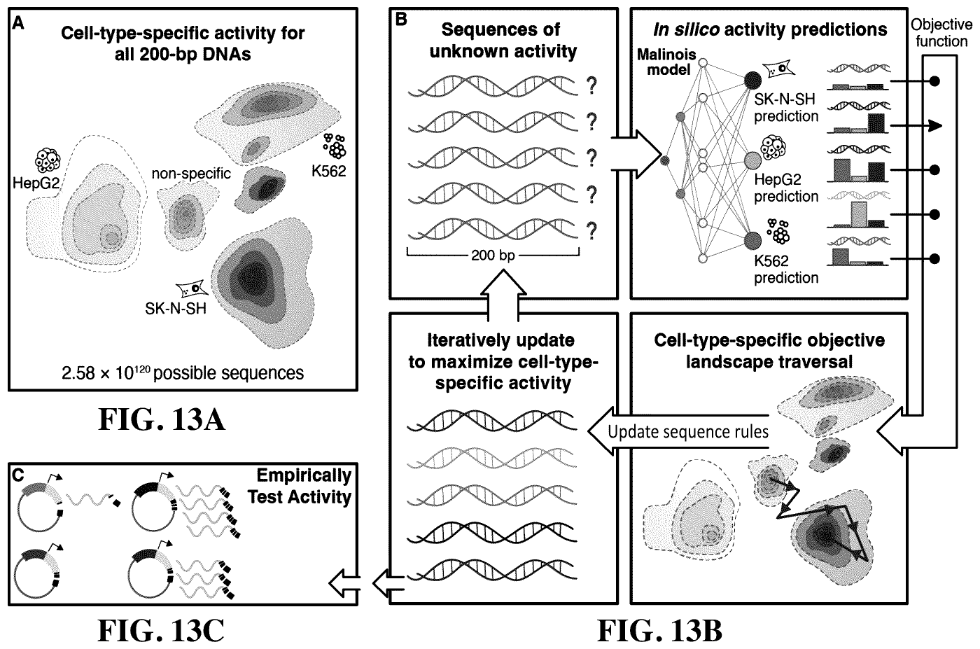

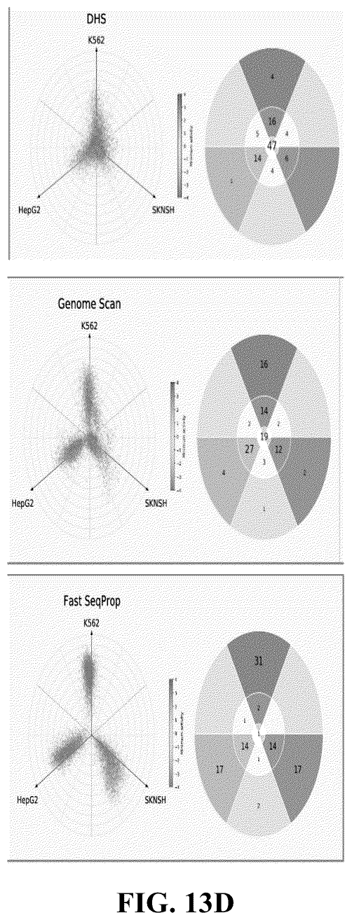

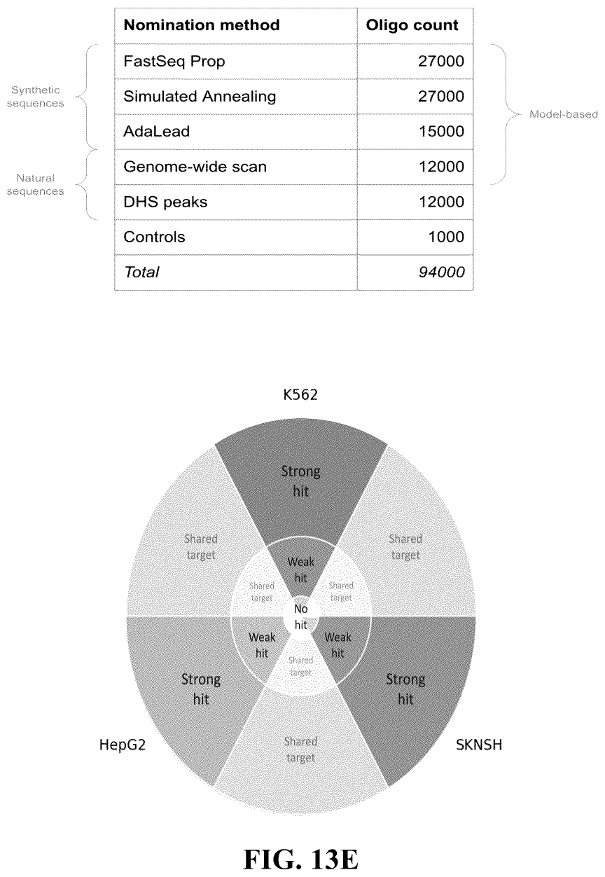

### Malinois Successfully Designs Cell-Specific Enhancers (FIG. 13A-13D)

Equipped with an accurate, cell-type informed surrogate model for regulatory function, Applicant next aimed to generate novel synthetic CREs with desired functions. To achieve this Applicant developed CODA (computational optimized DNA activity), a platform for machine-guided design of synthetic sequences for any objective. CODA follows an iterative set of three fundamental steps (FIG. 13A). Starting with a set of 200-mer sequences Applicant (i) predicted CRE activity of each sequence using Malinois. (ii) CRE activity predictions are combined by an objective function into a single fitness value which quantifies how well the sequence fulfills the design goals. (iii) The sequence set is modified in-silico to eventually optimize fitness. Applicant continued iterating until a batch of designed sequences reaches a fitness plateau.

Applicant deployed CODA to rationally design transcriptional enhancers with cell-type specific activity across our three tested cell lines, and empirically tested them. Applicant optimized cell specificity by expressing fitness as the minimum gap between predicted activity in the targeted cell-type and the two off-target cell-types. Applicant initialized random 200-mer sequences to start exploration in novel sequence space and iteratively update these to maximize fitness in silico using evolutionary, probabilistic, and gradient-based sequence design algorithms (FIG. 13B). Applicant generated 5,000 synthetic sequences predicted to be specific in each of K562, HEPG2, and SK-N-SH cells with CODA.

Applicant also compared how natural capable sequences were at driving cell-type specific activity versus synthetics. Chromatin accessibility is a common proxy for putative CRE activity, so Applicant identified 12,000 DHS-natural sequences' with cell-type specific DNAse signal in each of K562, HEPG2, and SK-N-SH cell lines (4,000 per line). Applicant then scanned the entire human genome for 200-mers predicted to be cell-specific by Malinois to identify ‘Malinois-natural sequences’, which notably takes <2 hours of compute time. Applicant selected 12,000 total sequences with the greatest on-target expression and minimal off-target expression in each of the three cell lines. Notably, few Malinois-natural sequences overlapped DHS-natural sequences in their own cell type (% k562, % hep, % SK), and were in predominately in repeat and X(sei analysis) elements of the genome). In total, Applicant proposed a library composed of 24,000 natural and 69,000 synthetic sequences. Applicant experimentally tested these sequences using MPRA in the three target cell types to empirically evaluate CODAs generative ability.

Empirical MPRA measurements were well correlated (Pearson's r≥0.86; Spearman's ρ≥0.89) with model predictions, and each class of sequences showed varying levels of success for cell-type specificity. To quantify the degrees of success for each approach we summarized cell type specific activity by measuring the distance between the on-target and off-target activities. Applicant defines success in achieving cell specificity when the log2FC separation between the maximum and minimum cell types is at least 1, and at least twice the separation between the median and the minimum. The success rate of the synthetic sequences ranged from 91% to 95%, while the Malinois-natural and DHS-natural sequences showed success rates of 75% and 41%, respectively (FIG. 13D). When increasing stringency between the on-target and minimum off-target to 4, synthetic sequences showed even greater performance gains compared to both classes of natural sequences (synthetic: 48%-65%; Malinois-natural: 22%; DHS-natural: 5%).

To understand the reason behind the performance differences, Applicant compared activity of on-target and off-target measurements between classes (FIG. 13E). Synthetic sequences consistently displayed greater separation between target and non-target cell types primarily due to repressive effects in non-target cell types (median off-target log2FC: synthetic −0.69; DHS-natural 0.41; Malinois-natural 0.09). Synthetic sequences also drove higher activity for on-target sequences when designed for expression in SK-N-SH (SK-N-SH median on-target log2FC: synthetic 3.20; DHS-natural 0.64; Malinois-natural 0.84). Together, this suggests a striking reservoir of genomic elements in the genome that can act as highly active and somewhat specific elements CREs, while DHS elements largely retain high levels of pleiotropy. Similarly, synthetic CREs, with no homology to the human genome, can drive the most consistently robust cell-specific activity through increases in on-target activity and off-target repression.

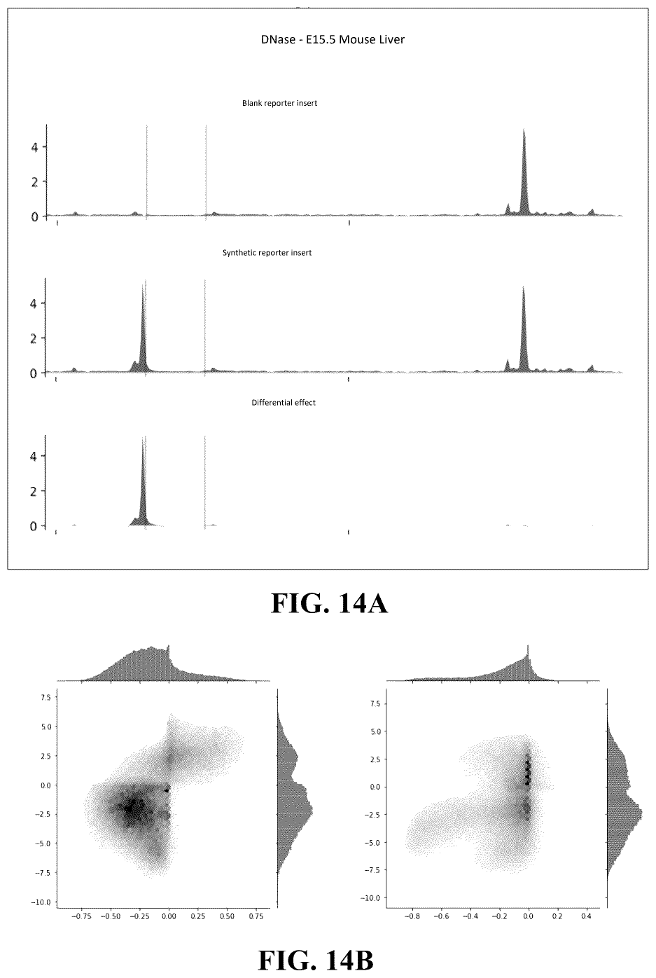

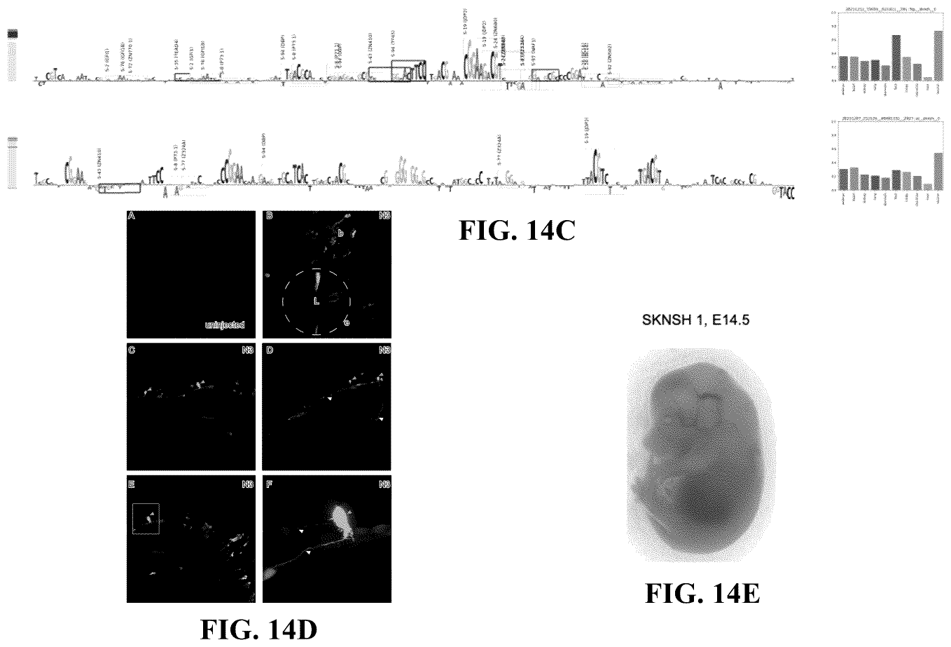

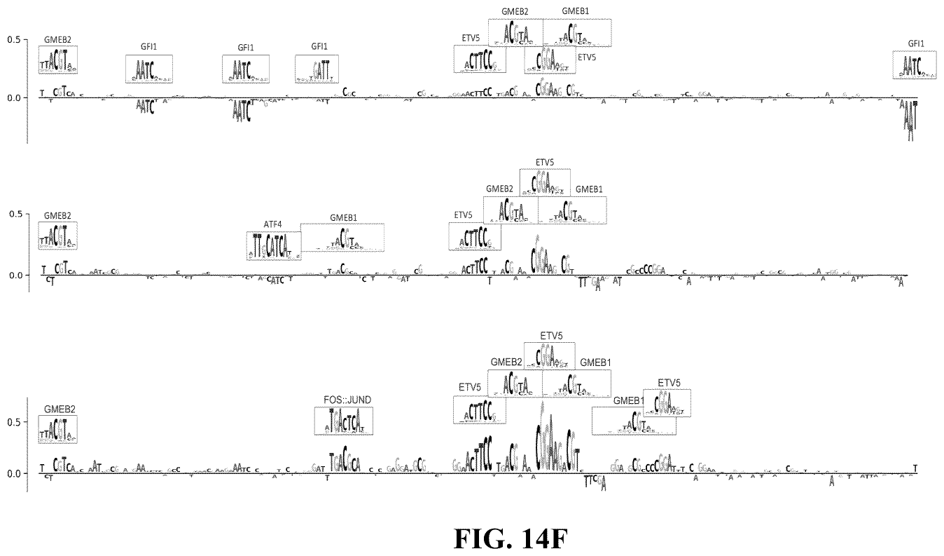

### Designed Synthetic CREs Drive Desired Cell-Type Specific Activity In-Vivo (FIG. 14A-14F)

To assess our synthetic CREs' specificity beyond an episomal reporter context in cell-lines, Applicant selected sequences for testing in an in vivo zebrafish model. Applicant first predicted in silico epigenetic features changes of Applicant's synthetic CREs when integrated into non-human genome in order to simulate cross-species, endogenous effects of candidate CREs (Enformer) (FIG. 14A). Applicant simulated a CRE's impact on DNAse and H3K27ac in 10 different tissue types, including hepatocytes and neurons to ensure agreement with MPRA empirical findings. Simulated tissue-type specificity for hepatocyte- and neural epigenetic features were well correlated with MPRA measurements overall (FIG. 14B). Using empirical MPRA results, in-silico tissue-specificity predictions, element vocabulary, and Malinois contribution scores, Applicant nominated three liver and three neuronal CREs for in-vivo characterization in zebrafish embryos (FIG. 14C-14F)

### Example 5—Machine-Guided Design of Synthetic Cell Type-Specific Cis-Regulatory Elements

The understanding of how CREs impact gene expression has been primarily derived from those elements that exist naturally in the human genome¹⁻⁴. Major efforts over the past decade have identified millions of putative CREs, yet these sequences generated by evolution represent only a small subset of possible genetic sequences and may not meet expression objectives favorable for therapeutic applications⁵⁻⁷. Indeed, 200 base pairs of DNA can encompass over 2.58×10¹²⁰ possible sequences, more combinations than atoms in the observable universe. This unexplored CRE sequence space, combined with our current poor understanding of the underlying principles driving CRE function, limit our ability to leverage CREs for clinical or biotechnological applications⁸. Bridging the gap in knowledge of ‘regulatory grammar’—the syntax of activating and repressing transcription factor (TF) vocabularies, their combinatorial effects, and higher order rules of TF cooperativity—has been a major goal of genomics for the past decade^{6, 7, 9-12}.

Recent advances are reshaping our ability to design CRE sequences with cell type-specific activity by overcoming three gaps in knowledge: (1) scalable methods to functionally characterize natural and synthetic CREs to produce generalizable insights (2) accurate ‘regulatory grammar’ models of how genetic sequences lead to CRE activity across cell types, and (3) the ability to repurpose predictive models for directed CRE generation. First, MPRAs can directly characterize CRE activity potential at-scale and across cell types¹³⁻¹⁸. Hundreds of thousands of CREs have been functionally characterized by MPRA, providing initial insights into regulatory syntax and transcriptional specificityl⁹⁻²³. Second, deep learning has emerged as an effective tool to accurately model the relationship between genetic sequences and biological phenotypes²⁴⁻³². While these sequence models are promising tools for the interpretation of genetic sequences^{27, 28, 31, 33}, they have largely been trained on, and predict, proxies of regulatory activity such as regions of open chromatin demarcated by DNAse Hypersensitivity sites (DHS), rather than direct CRE activity. Lastly, although computational models are millions of times faster than experimentation, they are incapable of global searches over all possible sequence combinations within the size of a typical human CRE. Efficient frameworks to generate sequences from predictive models could enable rational and interpretable design of candidate CREs^{4, 34-39, 34-41} designing synthetic CREs to drive cell type specificity in drosophila^{40,41}. However, synthetic CREs designed using predictive models are untested in vertebrates, and their effectiveness compared to natural sequences remains unknown.

Programmed, highly precise, cell type-specific transcriptional control CREs would contribute to development of specialized reporters, CRISPR therapeutics, gene replacement approaches, and more. In particular, advances in gene therapies offer a route to ameliorating a rapidly growing list of human genetic diseases, but their widespread use is hindered by a lack of robust, cell type-targeted delivery⁴². While current nanoparticle⁴³ and viral vector⁴⁴ technologies have shown some promise in better targeting of clinically actionable tissues like brain and muscle, they often display many undesirable cell type off-target effects^{45,46} Being able to fabricate synthetic CREs with programmable, highly tissue-specific functions could provide orthogonal tools for such clinical applications as well as basic research.

Here Applicant presents a method to engineer novel synthetic CREs capable of driving gene expression with cell type specificity. Applicant leverages innovations in modeling regulatory grammar across cell types, efficient sequence space searching, and the MPRA experimental system that can validate thousands of CREs in parallel. Applicant used a recently generated database of uniformly processed MPRA experiments which characterized an unprecedented number of CREs to train an accurate deep-learning model that can rapidly predict activity for any sequence in silico.

Coupled to sequence generation algorithms, Applicant deploys a model to generate thousands of cell type-specific, synthetic CREs, which we functionally validate using MPRAs and in vivo using mouse and zebrafish.

### Results

### Deep Learning Models can Accurately Predict DNA Cis-Regulatory Activity

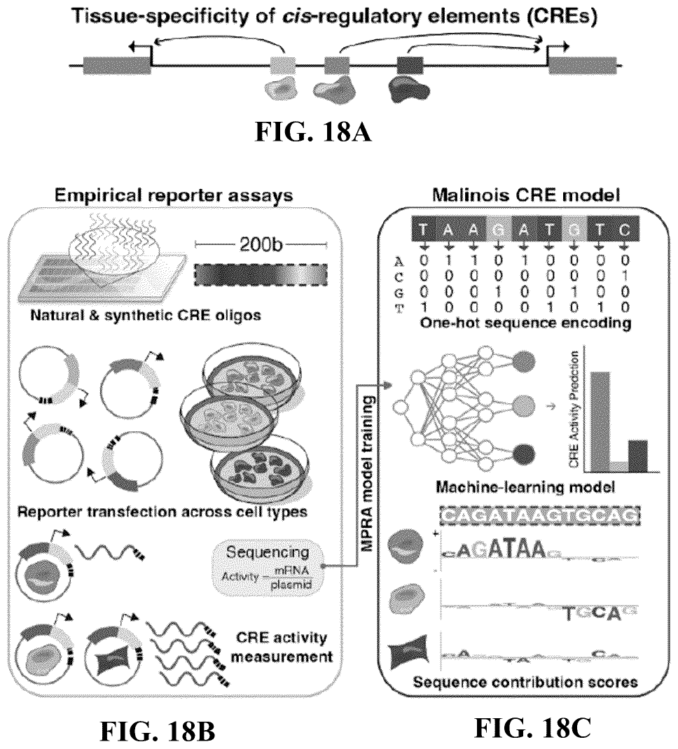

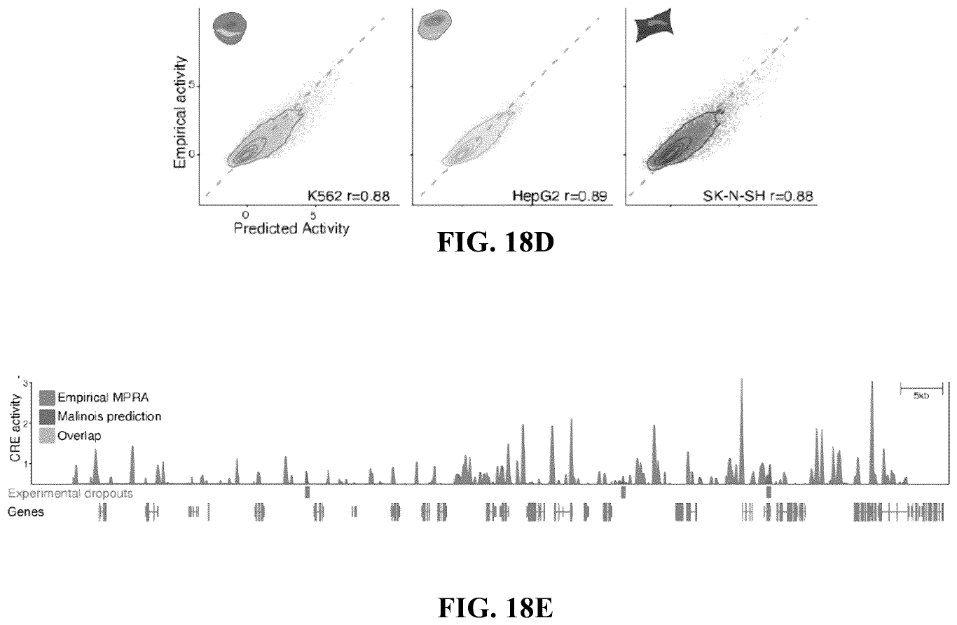

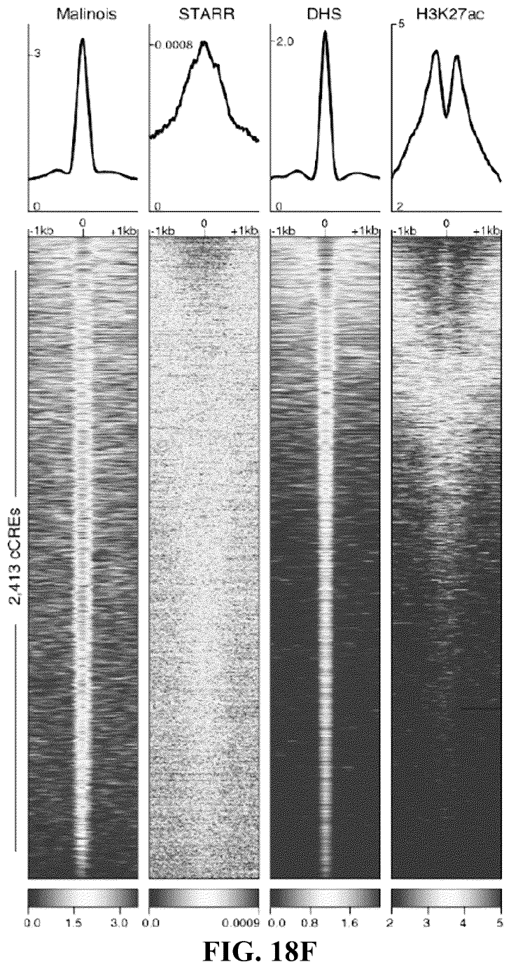

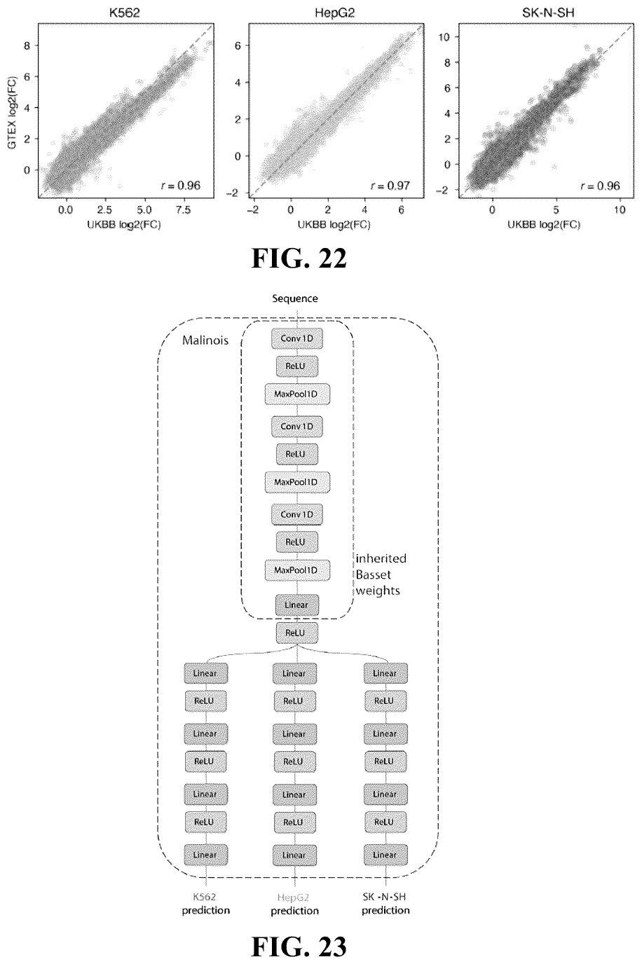

Applicant first built an accurate model of CRE activity from DNA sequence alone (FIG. 18A). While previous models of CRE activity have primarily used epigenetic states correlated to CRE function^{28, 29, 33, 47, 48}, Applicant trained the model on the regulatory output of 776,474 200-nucleotide sequences directly, as assayed by MPRA, a high-throughput reporter system that quantifies the effect of a given sequence on gene transcription (Supplementary Tables 1 and 2 of Gosai et al. “Machine-guided design of synthetic cell type-specific cis-regulatory elements” BioRxiv doi: doi.org/10.1101/2023.08.08.552077 (2023), which are incorporated by reference as if expressed in their entireties herein, Methods). These MPRAs were conducted by a single lab using a consistent experimental and analytical pipeline, yielding highly reproducible measurements (FIG. 22, Supplementary Table 2 of Gosai et al. “Machine-guided design of synthetic cell type-specific cis-regulatory elements” BioRxiv doi: doi.org/10.1101/2023.08.08.552077 (2023), which are incorporated by reference as if expressed in its entirety herein23, FIG. 18B). In total, Applicant collected functional CRE measurements from 155.3 Mbp of unique genomic sequence in each of three human cell types: K562 (erythroid precursors), HepG2 (hepatocytes), and SK-N-SH (neuroblastoma). These well-studied cell types are ideal for high-throughput method development and can provide useful insight for the growing body of experimental gene therapies that target blood cells⁴⁹⁻⁵² and neurons53, but that can induce toxicity in the liver⁵⁴⁻⁵⁶.

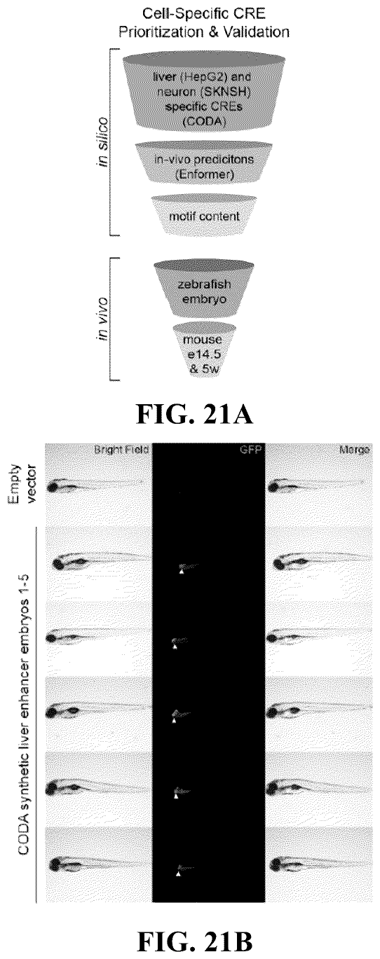

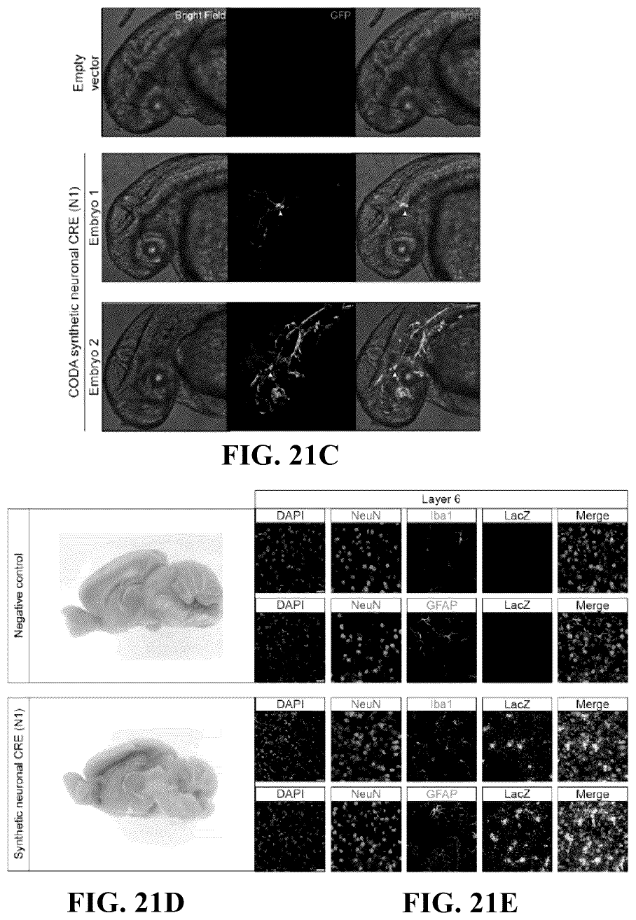

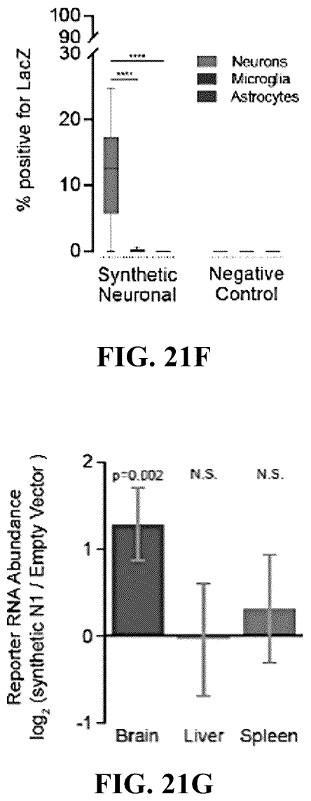

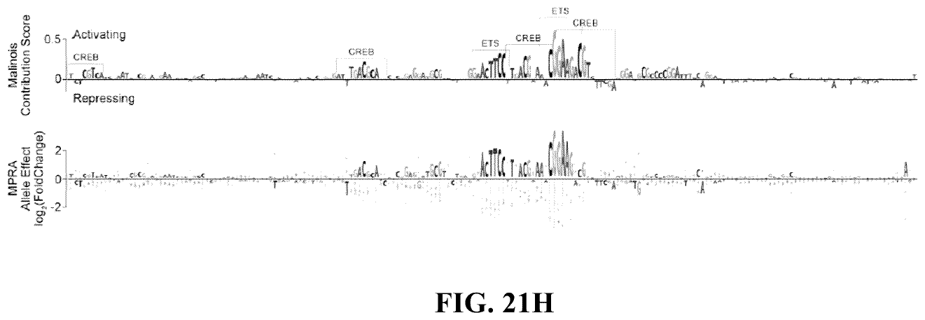

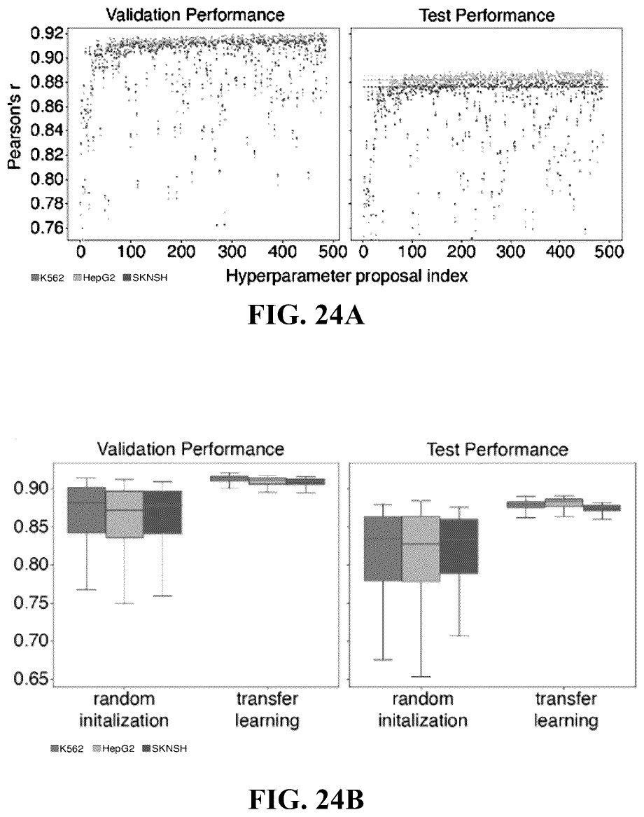

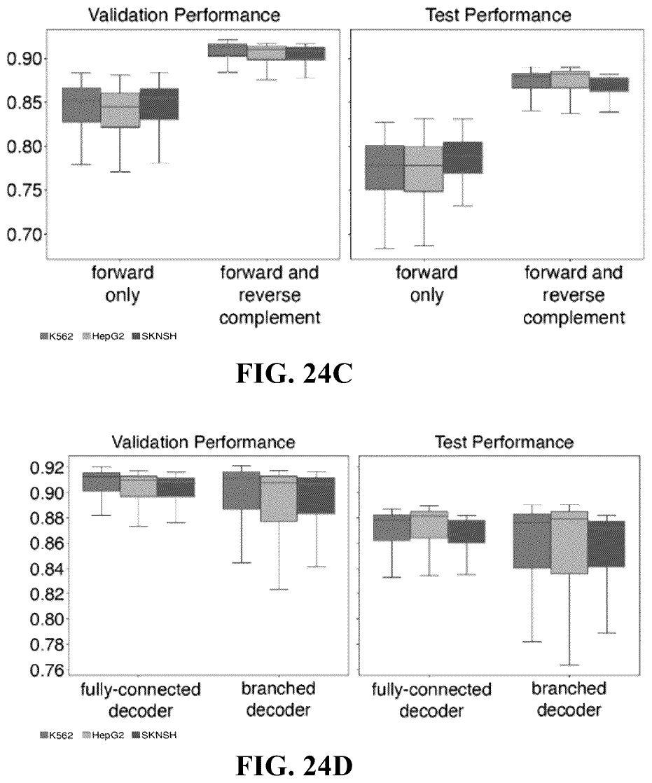

Applicant created Malinois, a deep convolutional neural network (CNN) for prediction of cell type-informed CRE activity of any arbitrary sequence as measured by MPRA. Applicant adapted architectural components from Basset47, a model of chromatin accessibility (FIG. 18C, FIG. 23, Methods), and leveraged Bayesian optimization57.58 to iterate over hyperparameter settings to identify a high performing model (FIG. 24A). Applicant observed several design choices that impacted the model including the use of transfer learning from Basset (FIG. 24B-24D), Table 8, Methods). Malinois accurately models episomal CRE activity across cell types. For sequences held out from training (62,582 elements on chromosomes 7 and 13), Malinois predictions in K562, HepG2, and SK-N-SH correlate highly with empirical activity measurements (Pearson's r 0.88-0.89; Spearman's ρ 0.81-0.83) (FIG. 18D) and demonstrate cell specificity on par with experimental results (FIG. 21A-21H).

TABLE 8

| Row ID | hepg2_test | hepg2_val | sknsh_test | sknsh_val | k562_test | k562_val |
|---|---|---|---|---|---|---|
| 1 | 0.8727607096904124 | 0.9023702169821205 | 0.8662966550841862 | 0.9030436309939325 | 0.8710547481505082 | 0.9091108526662749 |
| 2 | 0.8829122618904712 | 0.9121659955072344 | 0.8767985432132154 | 0.9105064959634173 | 0.8816199086256657 | 0.9131994196964852 |
| 3 | 0.8760190440542672 | 0.9059016695249689 | 0.8682576998529813 | 0.9033961814519633 | 0.871077827 | 0.9083479487926271 |
| 4 | 0.8602000996248133 | 0.8927564343693984 | 0.8560287694136948 | 0.8916253643152865 | 0.8574495872750535 | 0.8979196665504764 |
| 5 | 0.8872204060648252 | 0.9141043745428152 | 0.8795242368463076 | 0.9136023114949707 | 0.8837060274721964 | 0.9164280108651381 |
| 6 | 0.8772839256475958 | 0.9052793505653851 | 0.8729504595416628 | 0.9066545436743294 | 0.8767601547772628 | 0.9123551735695794 |
| 7 | 0.7172750088040758 | 0.7896191947547798 | 0.7424231234458162 | 0.8024699352153396 | 0.6953525217718264 | 0.7846896518822131 |
| 8 | 0.8865582136049526 | 0.9140934273573762 | 0.8791758430545711 | 0.9123511525154548 | 0.8829392904311977 | 0.917344915 |
| 9 | 0.8518581764181142 | 0.8873213240761841 | 0.8480725024109393 | 0.8882854706754312 | 0.849262372 | 0.891402367 |
| 10 | 0.8879041604926643 | 0.913636787 | 0.8796488164957837 | 0.9125278300885576 | 0.8844781152185158 | 0.9158190493845204 |
| 11 | 0.8868698842224502 | 0.913960693 | 0.8790759640476212 | 0.9130273598406775 | 0.8843350732649233 | 0.9169083559832679 |
| 12 | 0.8875094656868334 | 0.9167993268077582 | 0.8785901148276133 | 0.91612087 | 0.8843588200714718 | 0.9187093515358334 |
| 13 | 0.8707643954707355 | 0.9017628839659775 | 0.8644930861911574 | 0.9021857801860005 | 0.8688634581284607 | 0.9067224497619589 |
| 14 | 0.8379941095729764 | 0.8765800340819887 | 0.835313969 | 0.8789053896620658 | 0.839462908 | 0.8820670480995855 |
| 15 | 0.8837446537105634 | 0.912934734 | 0.8761086458857477 | 0.9107830856790234 | 0.8833054317792107 | 0.9154234019694723 |
| 16 | 0.886307328 | 0.9150344730008738 | 0.8779236965737272 | 0.9141664371089018 | 0.8836733452812793 | 0.9175031666194784 |
| 17 | 0.8854925423482046 | 0.9144008242641273 | 0.8769216883156161 | 0.9132057971427809 | 0.8822338864553634 | 0.9163553989606424 |
| 18 | 0.749684415 | 0.8242616666394046 | 0.7703629923345802 | 0.8347449639738642 | 0.7545714164798052 | 0.8319942139572228 |
| 19 | 0.45006885414850034 | 0.5739225559432466 | 0.45912348315380813 | 0.5720392176270276 | 0.46128560739519425 | 0.6092257750914258 |
| 20 | 0.8874348009909312 | 0.913657762 | 0.8781334858661598 | 0.9120931243759783 | 0.8822047048913345 | 0.915108523 |
| 21 | 0.8874974089207786 | 0.9153001589680574 | 0.8799048635979445 | 0.9138405577932908 | 0.884933073 | 0.9175309596472301 |
| 22 | 0.8853841554058235 | 0.9127779614615155 | 0.8767286281451296 | 0.9122064683112104 | 0.8811638763252756 | 0.9163647501539018 |
| 23 | 0.7883142289333331 | 0.8359471404537457 | 0.7910226254086367 | 0.8413283639414224 | 0.7886573852246793 | 0.8449914895089785 |
| 24 | 0.878991933 | 0.9073325677400969 | 0.8709180113008979 | 0.9072335650681844 | 0.8755591048499031 | 0.910430177 |
| 25 | 0.8829072689501629 | 0.910602155 | 0.8755119275404578 | 0.9096629156037342 | 0.878734956 | 0.9133502872528505 |
| 26 | 0.8798891111283765 | 0.9093497620806474 | 0.8744440268684319 | 0.9096316817400999 | 0.8761450437574916 | 0.9111795071186783 |
| 27 | 0.8402182756708936 | 0.8828026831154537 | 0.841517784 | 0.8857377661156078 | 0.8404787780859322 | 0.8890427340038672 |
| 28 | 0.8774009164819592 | 0.9059923044526169 | 0.8706665075881584 | 0.9052699094010204 | 0.8760876229620236 | 0.9110776453125085 |
| 29 | 0.8670592026308428 | 0.8995751365104625 | 0.8627193485657572 | 0.9018371439477235 | 0.8653126485660709 | 0.903134811 |
| 30 | 0.8712584698579198 | 0.9004806927667002 | 0.8663547329868146 | 0.8999219401057162 | 0.8689498239242466 | 0.9047309586315603 |
| 31 | 0.8740038221609642 | 0.9054506542356955 | 0.8683977168312563 | 0.9057777740459619 | 0.8706273129739781 | 0.9091506140934799 |
| 32 | 0.8779145541899129 | 0.9050223093396658 | 0.8690337864140001 | 0.9036082554860139 | 0.8755888085455772 | 0.9097818734966021 |
| 33 | 0.8485035855878132 | 0.8868061495079165 | 0.8475691312923571 | 0.8897672574785164 | 0.8526691220622721 | 0.8962938097060532 |
| 34 | 0.8821976377111931 | 0.9117457111394072 | 0.8760358915924614 | 0.9107496916300257 | 0.8795548292213996 | 0.9142868616223868 |
| 35 | 0.8823387949439869 | 0.9090974094613951 | 0.8754651377062914 | 0.9072912085215455 | 0.8788360920838098 | 0.9109706353296496 |
| 36 | 0.6532996002807422 | 0.7496447376853805 | 0.6763623291037939 | 0.7623185954075642 | 0.6356196053826335 | 0.7496825445611022 |
| 37 | 0.8816662480439068 | 0.9089247142986093 | 0.8746441906158419 | 0.9080532415214363 | 0.8774327042969601 | 0.9116995746313115 |
| 38 | 0.8087032626446956 | 0.8562786230392142 | 0.8156444772025295 | 0.8637400270158823 | 0.8133689422333245 | 0.8676839035114576 |
| 39 | 0.87576541 | 0.9064997935653973 | 0.8696550055708342 | 0.9052114481176474 | 0.8743647054022015 | 0.9098669207067415 |
| 40 | 0.7828640818166979 | 0.851407665 | 0.7864935675565013 | 0.8567867336627125 | 0.7712818798378311 | 0.8517644235474003 |
| 41 | 0.8738962187354928 | 0.9017787248819683 | 0.8658589589700573 | 0.8964257315510302 | 0.8670720554663269 | 0.9052799735544984 |
| 42 | 0.8842386392115397 | 0.9132729976927658 | 0.8775706182326437 | 0.912429676 | 0.8811578786203903 | 0.9163243258186788 |
| 43 | 0.2372154947216864 | 0.3279124525701941 | 0.3116266441331428 | 0.38234022313617927 | 0.19916982823367135 | 0.31405196232495675 |
| 44 | 0.8896077726162914 | 0.9169429671206552 | 0.8816485822205327 | 0.9151541695761632 | 0.8871000282120072 | 0.9185324298810799 |
| 45 | 0.777195283 | 0.8465258623275527 | 0.7911122929295958 | 0.8544326458452863 | 0.7799291675281569 | 0.8553021678231424 |
| 46 | 0.785287064 | 0.8376818005040244 | 0.7893156921853139 | 0.8416622382730217 | 0.7881778800720416 | 0.8481317568774608 |
| 47 | 0.8824104799348612 | 0.9115675422599356 | 0.8749461213257067 | 0.9106617083574421 | 0.8780965100161144 | 0.9140322759235732 |
| 48 | 0.8696034252088385 | 0.9035023599357495 | 0.8662530771501447 | 0.9027503707043563 | 0.8686557747290997 | 0.9064912621506873 |
| 49 | 0.8847543800905243 | 0.9118582130718039 | 0.877595295 | 0.9110616375454269 | 0.8813596352832518 | 0.9154924450725187 |
| 50 | 0.8563874137485092 | 0.8900180207195855 | 0.8572105972188744 | 0.8922382579600704 | 0.8572445290380311 | 0.898833196 |
| 51 | 0.879711744 | 0.9044090352509923 | 0.8701380747987875 | 0.9010183862846546 | 0.8728663959777812 | 0.9072212040487492 |
| 52 | 0.8884834363330306 | 0.9149277831835638 | 0.8799180174811505 | 0.9134969683916097 | 0.8862314741662736 | 0.9177061008048921 |
| 53 | 0.8868345052306688 | 0.9136045193202863 | 0.8792297338772791 | 0.9127659180148525 | 0.8833521423024157 | 0.9152574042358943 |
| 54 | 0.8841368642904769 | 0.910041274 | 0.8759157280234132 | 0.9082835040210039 | 0.8789459863678168 | 0.9132785823079452 |
| 55 | 0.8105054652856896 | 0.8493005176055346 | 0.8167211475481442 | 0.8551900843811201 | 0.8108551887032214 | 0.8545087336356187 |
| 56 | 0.8676814724560422 | 0.898159202 | 0.8622626348754634 | 0.8974069793021354 | 0.8670577734486667 | 0.9035907986666925 |
| 57 | 0.8800084118112147 | 0.9071524980795362 | 0.8730473585576125 | 0.9032992335792968 | 0.8785027801162909 | 0.9100525754706231 |
| 58 | 0.8878820477083755 | 0.9158813074946952 | 0.8795028017034068 | 0.9138464966747039 | 0.8870628695865518 | 0.9193127201487246 |
| 59 | 0.8882718476382749 | 0.9152804702711366 | 0.8809693997426431 | 0.9141173461544558 | 0.8894004291037501 | 0.9202224346583422 |
| 60 | 0.8825538596500269 | 0.910808659 | 0.8743926586873959 | 0.9110117357009029 | 0.8825216057007603 | 0.9149611025441269 |
| 61 | 0.883488585 | 0.9139616932383203 | 0.8774138283583178 | 0.9119446774646929 | 0.8801911908479996 | 0.916119827 |
| 62 | 0.7647896998817183 | 0.8361006455510753 | 0.7788444938865754 | 0.8436841855057483 | 0.764602512 | 0.8422659624163908 |
| 63 | 0.8872941788593693 | 0.9141581815723603 | 0.8793238367686612 | 0.9133939582756921 | 0.8860308502048281 | 0.9181475054564117 |
| 64 | 0.8866584893489695 | 0.9139266497393511 | 0.8782475379786119 | 0.9132562728568264 | 0.8857692568925369 | 0.9179696862719584 |
| 65 | 0.8711100361653635 | 0.9038173288710041 | 0.865775688 | 0.9030042970656025 | 0.8683008790637774 | 0.906312396 |
| 66 | 0.8812254425255762 | 0.9092484441265583 | 0.8733950426032386 | 0.9095321785745083 | 0.8801197229592602 | 0.9136806405729548 |
| 67 | 0.8798383502525634 | 0.9071735950635779 | 0.8719100445011604 | 0.9054525202548208 | 0.8771348283310139 | 0.9118897706187945 |
| 68 | 0.8769337208519533 | 0.904816898 | 0.8730642927090901 | 0.906495284 | 0.8772382794670566 | 0.9099558787997014 |
| 69 | 0.8891004269284071 | 0.9142890981771474 | 0.8789243392206121 | 0.9127831760199121 | 0.8850977823554255 | 0.9176014862598925 |
| 70 | 0.8886002153966591 | 0.9153698697400255 | 0.8805259736720636 | 0.9149319704763857 | 0.885570297 | 0.9174384083499916 |
| 71 | 0.888183234 | 0.91592815 | 0.8808264176657136 | 0.9151826780675814 | 0.8855239072624197 | 0.9194779084948937 |
| 72 | 0.8647431536288778 | 0.8944813926496896 | 0.8606074277454596 | 0.8947851889714971 | 0.8632960785995616 | 0.9003138469906504 |
| 73 | 0.7880604413338755 | 0.8334586495537764 | 0.7899970087628878 | 0.8391360712978579 | 0.7855396 | 0.8416918849913134 |
| 74 | 0.12655458619591578 | 0.13699074010078066 | 0.11174139426605151 | 0.14425530639622766 | 0.094254003 | 0.095264341 |
| 75 | 0.883714484 | 0.91148597 | 0.8761797025667508 | 0.9102282573306274 | 0.8807688444459445 | 0.9156250792772339 |
| 76 | 0.8526473530021743 | 0.8884834383308071 | 0.8506907475923591 | 0.8912981611413016 | 0.8504761789704862 | 0.8958447364797137 |
| 77 | 0.8373109058130586 | 0.8731303357064352 | 0.8355125208730908 | 0.8762572416692823 | 0.8386892577367271 | 0.8788875701366237 |
| 78 | 0.49835715851930684 | 0.6317761218693239 | 0.4579364308007235 | 0.5775879902512403 | −0.454238822 | −0.626913674 |
| 79 | 0.7923775013277073 | 0.8566669566335241 | 0.7984832302353776 | 0.8612493412625473 | 0.7905301915691305 | 0.859934864 |
| 80 | 0.8787422104203158 | 0.9050026701519731 | 0.872632082 | 0.90346241 | 0.8761176293545097 | 0.9099951172472126 |
| 81 | 0.8865841370533358 | 0.9137075924602335 | 0.876907375 | 0.9137692861480978 | 0.8839468712315638 | 0.916724999 |
| 82 | 0.8871716297452904 | 0.9150569659583733 | 0.8797663434973257 | 0.9138792336556856 | 0.8859123272255965 | 0.9178565784940799 |
| 83 | 0.8873024020640191 | 0.915389503 | 0.8792412416316926 | 0.913597695 | 0.8863025997763188 | 0.9181694795375224 |
| 84 | 0.881193573 | 0.9086072741930898 | 0.8758351743120987 | 0.9074790525585084 | 0.8792323788566058 | 0.9115510316920076 |
| 85 | 0.8867288576896275 | 0.9153433363315353 | 0.8790631612254549 | 0.9146197864834811 | 0.8838216865186693 | 0.9185403839042512 |
| 86 | 0.8776543435538786 | 0.9062961282331343 | 0.8708819101886193 | 0.9053637978570684 | 0.8736020692249984 | 0.9097364432245616 |
| 87 | 0.8848992648129845 | 0.912510821 | 0.8782217478149078 | 0.9114952759684298 | 0.8826043376534779 | 0.9160913501182057 |
| 88 | 0.8863120861084598 | 0.9133937520341933 | 0.8796295421294511 | 0.9117471759757076 | 0.8843557019884433 | 0.9166513922184565 |
| 89 | 0.88178964 | 0.9109050873687837 | 0.874884595 | 0.9090981557291941 | 0.8796498511410649 | 0.9134891023515475 |
| 90 | 0.7978993939825861 | 0.860165427 | 0.8023683847955763 | 0.8651357655429703 | 0.8006236084174996 | 0.865611316 |
| 91 | 0.8428659705710941 | 0.8826642994138282 | 0.8456708281603946 | 0.8867740547720788 | 0.8482627748476423 | 0.8944592269517676 |
| 92 | 0.8871478109281019 | 0.9143718104370828 | 0.8781028111576875 | 0.9118986816185554 | 0.883700154 | 0.9167849772370396 |
| 93 | 0.8849177388144907 | 0.9127656903542796 | 0.8756570368983068 | 0.9114850596739985 | 0.8816759708980435 | 0.9147343346674209 |
| 94 | 0.8885772045714847 | 0.9143541968942629 | 0.880551607 | 0.9123925741416195 | 0.8847224329050598 | 0.9169195029337951 |
| 95 | 0.7151905473896164 | 0.7730207754764593 | 0.7123660644248604 | 0.763169658 | 0.6517956064026985 | 0.7363801890294319 |
| 96 | 0.8059091511902348 | 0.8701128131018969 | 0.8144640581542428 | 0.8745266194421523 | 0.804573866 | 0.8735678535936486 |
| 97 | 0.8868602503318123 | 0.9152456502425044 | 0.8805785021335859 | 0.9143995798010985 | 0.8849445377180228 | 0.9186587731898723 |
| 98 | 0.8820327971909324 | 0.9120087711723333 | 0.8750244934245613 | 0.9118878726840629 | 0.8785933763768871 | 0.9133439812329274 |
| 99 | 0.8843612398771833 | 0.9129474398366458 | 0.8777094628698164 | 0.9089934690683037 | 0.8828255162795321 | 0.91576861 |
| 100 | 0.8502713180708886 | 0.8879882800017278 | 0.8490898794597794 | 0.8927268885216184 | 0.8474141847208723 | 0.8950373578956929 |
| 101 | 0.8847447960615804 | 0.9122127516023475 | 0.8776888727801349 | 0.9122953957525818 | 0.8805105618895659 | 0.9136911775550013 |
| 102 | 0.8803219280373458 | 0.9087270990035762 | 0.8751407823092955 | 0.9074786931994656 | 0.8774851320492872 | 0.9120552692983335 |
| 103 | | | 0.8369051925576133 | 0.8781271231853571 | 0.8425272866516043 | 0.8890468742065574 |
| 104 | 0.8825295571776159 | 0.9119248399515658 | 0.8753952660134204 | 0.9113740549887202 | 0.8801277278698226 | 0.9135748336449624 |
| 105 | 0.8853270552320385 | 0.9134532374477354 | 0.8767494594809756 | 0.9119094349103525 | 0.882939542 | 0.9154562242320551 |
| 106 | −0.096148653 | −0.096063225 | −0.048269454 | −0.065374859 | −0.080574571 | −0.079271049 |
| 107 | 0.8830269756318898 | 0.9105377718629456 | 0.8760017646800963 | 0.908695593 | 0.8819734535562721 | 0.9145229941576491 |
| 108 | 0.8862196581775211 | 0.9125253867635428 | 0.8798805408360133 | 0.9110786579588867 | 0.8831077231886039 | 0.9155558891998694 |
| 109 | −0.006082486 | 0.001342397 | −0.005124669 | 0.001835879 | | |
| 110 | 0.7850504367741831 | 0.8370186896367844 | 0.7983677957695507 | 0.8460862572065899 | 0.7864415987457241 | 0.8442334148485966 |
| 111 | 0.8832059832989514 | 0.913182188 | 0.8750852670376745 | 0.9132685795644647 | 0.8821266128336199 | 0.9167634065026955 |
| 112 | 0.8257347084289665 | 0.8777697814112302 | 0.827024424 | 0.8812240720756528 | 0.8240276004996325 | 0.8818750137752038 |
| 113 | 0.8561254362146946 | 0.8922109819494788 | 0.8518256858111861 | 0.8937006846591388 | 0.8550696358467533 | 0.8982170835906585 |
| 114 | 0.8878507617759566 | 0.9153015286894659 | 0.8791769497724178 | 0.9130513194217551 | 0.8864678123715051 | 0.9186601003654136 |
| 115 | 0.8855136386493853 | 0.9137379273920101 | 0.8784872816767539 | 0.9126016981027114 | 0.8830424380134944 | 0.9162916728563163 |
| 116 | 0.8853252961524211 | 0.9143562541311739 | 0.8790928025476201 | 0.9127435837720284 | 0.883205227 | 0.9167234631254761 |
| 117 | 0.8745649150579542 | 0.9039942541693158 | 0.8695277628805522 | 0.9034927588849715 | 0.8726221835901105 | 0.907869487 |
| 118 | 0.8824421117877868 | 0.9106421255086734 | 0.8744820958830464 | 0.9096738153629998 | 0.8775583587818256 | 0.91272078 |
| 119 | 0.8819255483674364 | 0.9110886524785231 | 0.8751315271386231 | 0.9109926207533183 | 0.8831759898932625 | 0.916085235 |
| 120 | 0.7559681112207924 | 0.8234314135176256 | 0.7706306667997397 | 0.8318970127645449 | 0.7506809967284873 | 0.8274715085171491 |
| 121 | 0.8601240983485215 | 0.8937316827407082 | 0.8577635559904824 | 0.8908796456201942 | 0.8611020253134347 | 0.9001433935999147 |
| 122 | 0.8181220071449421 | 0.8653444281590514 | 0.8285723692253619 | 0.8724294434838729 | 0.8241728779665737 | 0.8785825266918572 |
| 123 | 0.8866039972174499 | 0.9143081939406377 | 0.8778464279540947 | 0.9134569690146329 | 0.8842176447557584 | 0.9179387972412478 |
| 124 | 0.8861774248441048 | 0.912571835 | 0.875969393 | 0.911282885 | 0.8840173695792478 | 0.9166140578385583 |
| 125 | 0.8766389183170638 | 0.9086995290896879 | 0.8709725329130175 | 0.9080945608868743 | 0.8748460970051342 | 0.9128900699312585 |
| 126 | 0.8739670582706272 | 0.9044474148838262 | 0.8709284255982257 | 0.9060042261831447 | 0.8740800109805953 | 0.9101331237628105 |
| 127 | 0.8832982613646427 | 0.9111314447930448 | 0.8750620942845602 | 0.9097393298930756 | 0.8800054593422586 | 0.912603547 |
| 128 | 0.8784379438763047 | 0.9082137555447065 | 0.874016707 | 0.9070275860206654 | 0.8775675428382302 | 0.911969306 |
| 129 | 0.8798525473065419 | 0.9114941391373034 | 0.8737865631831893 | 0.9115753683601925 | 0.8795106115426012 | 0.9150444899009623 |
| 130 | 0.8277918161572724 | 0.8711654755801357 | 0.831542837 | 0.8771858627048504 | 0.8256880518100733 | 0.8779160987012223 |
| 131 | 0.8880536508237296 | 0.9143623867840099 | 0.8792546780802706 | 0.9136787723189721 | 0.8845021395699999 | 0.9186407759240017 |
| 132 | 0.885721044 | 0.9127277099621277 | 0.8771822020025432 | 0.912169604 | 0.8817775632175835 | 0.9151955782172091 |
| 133 | 0.8849523920117128 | 0.9119915156006273 | 0.8777028716613521 | 0.9114836297958364 | 0.880985143 | 0.9149718839000678 |
| 134 | 0.884937363 | 0.9142379794460567 | 0.8771586189908287 | 0.9124401366225333 | 0.8838074284053874 | 0.9165033147659187 |
| 135 | 0.871612791 | 0.9023856183460854 | 0.8648395841631601 | 0.9034046789739982 | 0.8686660565142836 | 0.9085197080443681 |
| 136 | 0.8351776945949962 | 0.8760994398360997 | 0.8437450711088971 | 0.8837206238172866 | 0.8406140125621318 | 0.8877632220752159 |
| 137 | 0.791003938 | 0.8334911323239773 | 0.8006938120447994 | 0.8400231436788064 | 0.7891444723129285 | 0.8418248415526155 |
| 138 | 0.8608718498668835 | 0.891167444 | 0.8551545629847155 | 0.8919674310135794 | 0.8578420365590266 | 0.8989364425737191 |
| 139 | 0.8785708266151431 | 0.9064899657185284 | 0.8723777417456042 | 0.9067241798868677 | 0.8755814036265919 | 0.9116895113250554 |
| 140 | 0.868332067 | 0.8967500119927714 | 0.8621574059851782 | 0.8980577177368886 | 0.8653601771260271 | 0.901918833 |
| 141 | 0.883858026 | 0.9119070508558434 | 0.8765832304893915 | 0.9098389012035215 | 0.8806043978372388 | 0.9136389295749284 |
| 142 | 0.8868487596584258 | 0.9171177071905521 | 0.8797205481029394 | 0.9149722648905153 | 0.8851429353024711 | 0.9191002070783062 |
| 143 | 0.7676264798388313 | 0.8183446327630037 | 0.7732799298558999 | 0.8231535193692732 | 0.7589912489049077 | 0.8212253870992278 |
| 144 | 0.8868709496563278 | 0.9152712252665469 | 0.8794221658945084 | 0.9148842104693391 | 0.8835222560558855 | 0.9183333884950209 |
| 145 | 0.8724366162163056 | 0.9061239145218627 | 0.868199428 | 0.9067603221704345 | 0.8731015185149407 | 0.9089784248872788 |
| 146 | 0.7682673997202916 | 0.8349481168572382 | 0.7798980628010465 | 0.8429116350523042 | 0.7652511522561778 | 0.8400381022086305 |
| 147 | 0.8784306557846269 | 0.9069501441346381 | 0.8724025065521366 | 0.907766603 | 0.8758775651828399 | 0.9123780608880231 |
| 148 | 0.8803228764266189 | 0.9085638725241709 | 0.8722190394385564 | 0.9075185609814609 | 0.8784110908928247 | 0.9114325083026383 |
| 149 | 0.8808228697871596 | 0.9089883380315164 | 0.8736875602778295 | 0.9070021977465159 | 0.8793863001575674 | 0.9130994059609536 |
| 150 | 0.8823316723359218 | 0.9126747359511825 | 0.8744612702232837 | 0.9120479209585918 | 0.8786452191289458 | 0.9162632736034526 |
| 151 | 0.8790014748347275 | 0.9074602842335756 | 0.8719239416554134 | 0.9074836630576328 | 0.8753092500654471 | 0.9116556170358111 |
| 152 | 0.8182553171403061 | 0.8734986411666381 | 0.8235875056550787 | 0.8785040390049269 | 0.8143171513739984 | 0.8781698546960421 |
| 153 | 0.8720711838878095 | 0.9016539181017218 | 0.8658427921433182 | 0.9005679240008758 | 0.8703866699549309 | 0.9081605607446659 |
| 154 | 0.8743844093665513 | 0.9030825145699075 | 0.8664833166183218 | 0.9006426741278191 | 0.8674458829850936 | 0.9060302263825317 |
| 155 | 0.8601520911799643 | 0.8938905813964938 | 0.8561585405941735 | 0.893931093 | 0.8580758077644285 | 0.8980461869046266 |
| 156 | 0.8681528924710827 | 0.9025903971914155 | 0.8637867672936159 | 0.9032845101264936 | 0.8664425072467499 | 0.907427604 |
| 157 | 0.8834009760694063 | 0.9105012465528841 | 0.8763806104628045 | 0.9091181347794801 | 0.8805777879172659 | 0.912737867 |
| 158 | 0.8793658445068974 | 0.9088361653962965 | 0.8714796996541669 | 0.9065842557494712 | 0.8773864248276793 | 0.9121614115249402 |
| 159 | 0.8854735157295515 | 0.9128521378394302 | 0.877188674 | 0.9123736071799761 | 0.8823499968827779 | 0.9157911391977511 |
| 160 | 0.8000844210250868 | 0.8392646499791288 | 0.8049214537140796 | 0.8428753383801874 | 0.8009057452565403 | 0.8444195658854998 |
| 161 | 0.7168860624202653 | 0.7952976244773243 | 0.7322781928350794 | 0.8043661674813721 | 0.7103641015426996 | 0.8028691101530928 |
| 162 | 0.8488668428823222 | 0.8835843274356866 | 0.8494547817274364 | 0.8894018798489345 | 0.8527860097436609 | 0.8942820488763618 |
| 163 | 0.88011468 | 0.9109389533705271 | 0.8731843142340572 | 0.9107497605961268 | 0.8780334173358251 | 0.91345538 |
| 164 | 0.886157702 | 0.9123981229600648 | 0.8780917517543672 | 0.9111031283517026 | 0.8823084340510583 | 0.9148015933725898 |
| 165 | 0.8812462775647119 | 0.9095931731006285 | 0.8749694504185039 | 0.9087070515901438 | 0.8783005080866043 | 0.9127681543685969 |
| 166 | 0.5264865997882515 | 0.6436861882914962 | 0.5404357272772714 | 0.6475272398928443 | 0.48806850622597836 | 0.6246097699271732 |
| 167 | 0.8690793252751395 | 0.9008093871170179 | 0.8643273091186245 | 0.8995473248814524 | 0.8664126790687131 | 0.9050039577208736 |
| 168 | 0.8424341829815356 | 0.8777482110799202 | 0.8451661258867643 | 0.8830735074700533 | 0.8465234584487288 | 0.8868681258700094 |
| 169 | 0.7957489228859658 | 0.8382547980235117 | 0.8030535693900411 | 0.8424357722605457 | 0.8005554415918777 | 0.845728424 |
| 170 | 0.8305432246908296 | 0.8724443354456828 | 0.8366950394590486 | 0.8792651457857956 | 0.8367843191816258 | 0.8836748135823373 |
| 171 | 0.8773688366136263 | 0.9058597272623041 | 0.8711428916223632 | 0.9059304938733218 | 0.8740882410116391 | 0.910958452 |
| 172 | 0.8819489571383764 | 0.9084936495807954 | 0.8750837694877642 | 0.908331853 | 0.8784210077974631 | 0.9109028838805238 |
| 173 | 0.8854500239482768 | 0.9126301001868722 | 0.8790960294274902 | 0.9125598106670907 | 0.8859107065799001 | 0.9181179227941765 |
| 174 | 0.8858239849807148 | 0.9143932237221245 | 0.8777652714016287 | 0.9124838726426578 | 0.8844944086800206 | 0.9174977363219565 |
| 175 | 0.8861490203576999 | 0.9134715370876585 | 0.8802915866031998 | 0.9123968104954427 | 0.8844083033899622 | 0.9149651139020932 |
| 176 | 0.884406226 | 0.9134494333816026 | 0.876332981 | 0.9128701693026822 | 0.8828019492699059 | 0.9157530042468196 |
| 177 | 0.8761432847564872 | 0.9044282173739522 | 0.8694217779508349 | 0.9039135964587545 | 0.8744366666054202 | 0.9094965261044122 |
| 178 | 0.8105590319069711 | 0.8653375675285894 | 0.8198267245232405 | 0.8748772610582263 | 0.8116721851510772 | 0.8733377972033372 |
| 179 | 0.8774601019847315 | 0.904783875 | 0.871094472 | 0.9048845395439017 | 0.8762933364940754 | 0.9104528756417885 |
| 180 | 0.8863331881887264 | 0.9137893787878072 | 0.8790661658391673 | 0.9127633452745387 | 0.8851584983952554 | 0.9167048717768407 |
| 181 | 0.8827905533619083 | 0.9107892048039111 | 0.8751933231521709 | 0.9103871244028596 | 0.880332401 | 0.9145705780562711 |
| 182 | 0.7445226018593173 | 0.7937398065438392 | 0.7551894298718528 | 0.7989224873454042 | 0.7271467985834169 | 0.79601996 |
| 183 | 0.8850122784372281 | 0.9147337736375456 | 0.8774061601836669 | 0.9143759618289692 | 0.8836099075840811 | 0.9186460979061695 |
| 184 | 0.8811219886065808 | 0.9091218886632898 | 0.874357055 | 0.9079544687653308 | 0.8803796092273142 | 0.9128247681852116 |
| 185 | 0.8764115862087715 | 0.9059474484865375 | 0.8712664942645504 | 0.9039850551428085 | 0.875162845 | 0.9088027148721838 |
| 186 | 0.8843024391898462 | 0.9115150080999314 | 0.8768374784241277 | 0.9101255071726109 | 0.8803544909253042 | 0.9132149779968767 |
| 187 | 0.6173297218484419 | 0.7113320237395442 | 0.610437563 | 0.7000200726558091 | 0.5829899605294913 | 0.6969406950881436 |
| 188 | 0.8815675748916596 | 0.9104863345221201 | 0.874859471 | 0.9090860569883255 | 0.878592068 | 0.9140948747316013 |
| 189 | 0.8738166321986396 | 0.905703163 | 0.8681219422112662 | 0.9050634142081854 | 0.8732110247352065 | 0.9098218115065744 |
| 190 | 0.867576369 | 0.9016142673945473 | 0.8661903140070153 | 0.9043734505525868 | 0.8674614100257892 | 0.905963706 |
| 191 | 0.8755623426914927 | 0.9028156997581598 | 0.8671203613997899 | 0.8990033359012829 | 0.8685639501624334 | 0.9033667306524468 |
| 192 | 0.886523151 | 0.9154153628014748 | 0.8777329992858938 | 0.9144890021769945 | 0.8845266635349487 | 0.918637738 |
| 193 | 0.867517374 | 0.9000015177265177 | 0.8621246030632095 | 0.9000694240308285 | 0.8648623019993207 | 0.9037997156873334 |
| 194 | 0.8613879340017624 | 0.8938097342023235 | 0.8552660525966168 | 0.8935142650799537 | 0.8542264422299745 | 0.8976078978132292 |
| 195 | 0.8865392719447417 | 0.9152006036885242 | 0.8778336663784773 | 0.9133430949160379 | 0.8825530215795463 | 0.9174866005805966 |
| 196 | 0.877456612 | 0.9052787696842415 | 0.8716686601302205 | 0.9029939050378339 | 0.8766222002669161 | 0.909942221 |
| 197 | 0.8839537727206863 | 0.913293148 | 0.8777857084250813 | 0.9123388264919133 | 0.8813410670356624 | 0.9149866156715569 |
| 198 | 0.8791241566967701 | 0.9074879510973085 | 0.8713981095579012 | 0.9050163274719455 | 0.8755606574999876 | 0.9118661043995522 |
| 199 | 0.8844295353579501 | 0.9119413024367884 | 0.8749405406279706 | 0.9107393602472325 | 0.8815141134897808 | 0.9165009141070961 |
| 200 | 0.8844063708845767 | 0.912891795 | 0.8777616997552413 | 0.9130155945462006 | 0.8819753889412787 | 0.915293647 |
| 201 | 0.8821097936958265 | 0.9135330588471775 | 0.875821167 | 0.9106982465923026 | 0.8796927494386051 | 0.9160537852342492 |
| 202 | 0.8238034924221669 | 0.8702946347222469 | 0.8319166244791009 | 0.8783025760662051 | 0.8328514851635482 | 0.8835130515912347 |
| 203 | 0.8453118865654138 | 0.8837549064692155 | 0.8451331981051088 | 0.8869118853811047 | 0.8458857388057015 | 0.8907418146738355 |
| 204 | 0.8790658961373827 | 0.9078176205379811 | 0.8706690494784289 | 0.9056950362274598 | 0.8732415108128062 | 0.9100326918183269 |
| 205 | 0.8852739101845082 | 0.9126204913707641 | 0.8774677749510066 | 0.9122568975659556 | 0.8813739720037301 | 0.9160966626726293 |
| 206 | 0.8870046723407738 | 0.9157723348819597 | 0.8788978320783958 | 0.9135498619800404 | 0.8819707953045837 | 0.9172719652033752 |
| 207 | 0.5869558985545191 | 0.6718870157318693 | 0.6013098570218898 | 0.6814770684911522 | 0.5581895523787554 | 0.6659815691135651 |
| 208 | 0.8801533300891706 | 0.9096027904375192 | 0.8743147093917611 | 0.9090922834334635 | 0.8787707268255442 | 0.9139422486745139 |
| 209 | 0.8884275662691602 | 0.9146906362274576 | 0.8799542795581392 | 0.9133504303685467 | 0.884935769 | 0.9168875173299265 |
| 210 | 0.8495234292056555 | 0.8890458572790809 | 0.8507708788646315 | 0.8901378836069922 | 0.8523329941719744 | 0.8970142187416659 |
| 211 | 0.8758743902349652 | 0.9062261093337943 | 0.8701516323749373 | 0.9052411132280471 | 0.8731804266691234 | 0.9086344596077562 |
| 212 | 0.8828885326745721 | 0.9109654010413699 | 0.8745581855935172 | 0.9086186823951419 | 0.8801370924366712 | 0.9139717982116752 |
| 213 | 0.7441132980953604 | 0.805015463 | 0.7499739958485879 | 0.8074915270112197 | 0.7298802122167996 | 0.8064643673563155 |
| 214 | 0.8880019443005265 | 0.9145763102460922 | 0.8796841309289529 | 0.9128975419830091 | 0.8846414958160915 | 0.9154741459559808 |
| 215 | 0.7917806757277409 | 0.856249636 | 0.7898732968505829 | 0.8571295486442988 | 0.7819419610644953 | 0.8542067700523492 |
| 216 | 0.8783920578265954 | 0.9061138714995194 | 0.871693978 | 0.9053938048962611 | 0.8766454744180825 | 0.9107162076539427 |
| 217 | 0.8840686759504988 | 0.9120184418818906 | 0.8753572186118665 | 0.9110317003196549 | 0.8822258316569909 | 0.9159212851302547 |
| 218 | 0.8880018205455782 | 0.9127652283610255 | 0.8807446187829514 | 0.9124244913622378 | 0.8839968709230002 | 0.9151060792053604 |
| 219 | 0.8876983205989976 | 0.9142959975942472 | 0.8795145523474759 | 0.9126114915013619 | 0.8846205194559379 | 0.9162693774367507 |
| 220 | 0.8854928407090121 | 0.9125064935008624 | 0.8766824490966256 | 0.9111206184702709 | 0.8818288915574415 | 0.9145891593219646 |
| 221 | 0.8862288347017776 | 0.913614027 | 0.8787062110192714 | 0.9114537193754169 | 0.883002649 | 0.9166588188174669 |
| 222 | 0.8869394945753093 | 0.9128836225952521 | 0.8791029967734251 | 0.9098113778352576 | 0.8850518552132929 | 0.915038765 |
| 223 | 0.7993699427477562 | 0.8583801812204079 | 0.8046107960441478 | 0.8617748718904504 | 0.8008425253023688 | 0.8664796232467025 |
| 224 | 0.8838715680989864 | 0.9126204907710762 | 0.8769189994379873 | 0.9112977787293184 | 0.8816727286823265 | 0.9142335467829465 |
| 225 | 0.8345797716796555 | 0.8771296187194395 | 0.8420906442945666 | 0.8865958756001842 | 0.8438223124140853 | 0.893192473 |
| 226 | 0.8347155533298598 | 0.877269734 | 0.8408158634956439 | 0.8831296785912044 | 0.8412719535805633 | 0.8880137160269896 |
| 227 | 0.8583392421341367 | 0.8934727848860474 | 0.8521809188450056 | 0.8954802598667544 | 0.8569995749126422 | 0.8967723170770674 |
| 228 | 0.8855845615373648 | 0.9146351394639162 | 0.8761826453893997 | 0.9147303631108659 | 0.8848352826540726 | 0.9179770147860994 |
| 229 | 0.8307254173803507 | 0.8716809817494628 | 0.8390230656427091 | 0.8796536985326415 | 0.8369819852635391 | 0.881847936 |
| 230 | 0.8279588472496837 | 0.8728033120491824 | 0.8327966682258845 | 0.8790549544423505 | 0.8346001670084192 | 0.8862356338237233 |
| 231 | −0.439316469 | −0.561099752 | 0.42803354453440456 | 0.5456162097979104 | −0.41308367 | −0.542181753 |
| 232 | | | | | | |
| 233 | 0.8867490487360757 | 0.9144034991437826 | 0.8783564701766383 | 0.9131514093812355 | 0.8851061907221465 | 0.9182383121969014 |
| 234 | 0.8058586619106511 | 0.8651029846740961 | 0.8162788298640982 | 0.8717295480931181 | 0.8044711975401582 | 0.8709640572296473 |
| 235 | 0.8869738393882433 | 0.9147580459131073 | 0.8783968578482294 | 0.9136034460096525 | 0.8871119776122727 | 0.9187749101259504 |
| 236 | 0.829719025 | 0.8817251530915746 | 0.8295062801987965 | 0.8833949663759608 | 0.8274377804801181 | 0.8833315413885732 |
| 237 | 0.8820221667825343 | 0.9091768289120615 | 0.8760368044439006 | 0.9069970470987867 | 0.8800279932337367 | 0.9116824149071254 |
| 238 | 0.8869860947817128 | 0.9144800848358102 | 0.8803056960020819 | 0.9132277015462622 | 0.8832330619535378 | 0.9174618466948278 |
| 239 | 0.88735798 | 0.9151203328095615 | 0.8801132837498626 | 0.9144124381587583 | 0.8849192250411598 | 0.9184830345889631 |
| 240 | 0.8766191978895803 | 0.9064228798235539 | 0.8697662020472534 | 0.9055303884304635 | 0.8753926274144609 | 0.9108338407107146 |
| 241 | 0.8495967590834071 | 0.8861862412820251 | 0.8530764911561569 | 0.8906148926180391 | 0.8545620053192072 | 0.8965646636173529 |
| 242 | 0.8878537951163079 | 0.9147506555421423 | 0.8794413897429038 | 0.9138618508118337 | 0.8841738210277906 | 0.9179490653619664 |
| 243 | 0.8839643355338083 | 0.911473549 | 0.8765741609971475 | 0.9097394734815256 | 0.8806087432256662 | 0.9139293180621164 |
| 244 | 0.8280627062205019 | 0.8693623774733631 | 0.827706602 | 0.8753257569177223 | 0.8257605759445138 | 0.8741302930161103 |
| 245 | 0.6331136541407403 | 0.7162835536711509 | 0.6567664397032078 | 0.7311462771578534 | 0.6058315901730239 | 0.7083698290091064 |
| 246 | 0.8819239768437126 | 0.9098597144474426 | 0.8746717249460142 | 0.908975859 | 0.8805003371900909 | 0.9127901992289261 |
| 247 | 0.7059356066657102 | 0.779168353 | 0.7177316422118203 | 0.7826290619131127 | 0.6750830510638903 | 0.7674331013070349 |
| 248 | 0.8826226578833272 | 0.9124050332356803 | 0.8765954184400073 | 0.9113355491920452 | 0.8810568094071115 | 0.916633098 |
| 249 | 0.8227039780774231 | 0.8691349084788298 | 0.823587197 | 0.8785764190467329 | 0.8225537051463401 | 0.8760766002693443 |
| 250 | 0.7986440586750944 | 0.8587903880413035 | 0.7991563225224514 | 0.8626376429402801 | 0.794567509 | 0.8621723353752333 |
| 251 | 0.8796809910075176 | 0.9068019887364809 | 0.8720989215951571 | 0.9045358864351374 | 0.8737422029050312 | 0.9096715314687526 |
| 252 | 0.8429468358738215 | 0.8811994072682287 | 0.8402019609410939 | 0.885647077 | 0.8442537316675468 | 0.8879660341777088 |
| 253 | 0.8855010218714935 | 0.9119419740003815 | 0.87753209 | 0.9114073253310209 | 0.8831917038512848 | 0.9152530022392552 |
| 254 | 0.8145007204642153 | 0.855366806 | 0.8268731956637879 | 0.8673639093025516 | 0.8262570628215099 | 0.8734486509870394 |
| 255 | 0.6864064355226529 | 0.7707965546157076 | 0.7072630712251083 | 0.7812576164398447 | 0.6832010126368018 | 0.7791618809240487 |
| 256 | 0.8886991714465177 | 0.9166399400251888 | 0.8793946652390581 | 0.9158011842159559 | 0.8855188325132922 | 0.9195172250500635 |
| 257 | 0.8530784654477285 | 0.8884221111718403 | 0.8488330519772795 | 0.8884906241694294 | 0.8518509181394994 | 0.8948103942132264 |
| 258 | 0.878408008 | 0.9054311493797875 | 0.8713215066299628 | 0.9020927312573279 | 0.8721795642152035 | 0.9075382530188072 |
| 259 | 0.874401955 | 0.9027762618563872 | 0.8662314007212377 | 0.9021323551977032 | 0.870445467 | 0.9064232467824295 |
| 260 | 0.8873790356543387 | 0.9132553896942062 | 0.878015362 | 0.9112046919080836 | 0.883417728 | 0.913798125 |
| 261 | 0.8798845330780212 | 0.9089073649172661 | 0.8711069695363091 | 0.9059886010350424 | 0.8725632236354643 | 0.9102687667085038 |
| 262 | 0.8833507098264393 | 0.9120684574290919 | 0.8754354388839496 | 0.9100458456187764 | 0.879042041 | 0.9134724318128621 |
| 263 | 0.8884850997699816 | 0.9169619516731364 | 0.8783768172055709 | 0.9156784180761367 | 0.8853621920399004 | 0.9200844935228045 |
| 264 | 0.8792807789847199 | 0.907317323 | 0.8704724937096213 | 0.9067551679157475 | 0.875782633 | 0.9103259778154453 |
| 265 | 0.8775738184777071 | 0.9057699880285084 | 0.8709588649687773 | 0.9054190827990406 | 0.8750430082673605 | 0.9105758833924847 |
| 266 | 0.8772920159181273 | 0.9066794176601891 | 0.8679342036318828 | 0.9030754503085177 | 0.8707872692459061 | 0.9096427943742941 |
| 267 | 0.8826236190916266 | 0.9097302179141353 | 0.8760404060462288 | 0.9092504794372949 | 0.877427862 | 0.9121407965466525 |
| 268 | 0.8545548262749577 | 0.891146354 | 0.852951672 | 0.8934641697913283 | 0.8555262216556254 | 0.8989878282697018 |
| 269 | 0.8705081446155568 | 0.8984816648135532 | 0.8643172182294075 | 0.8992443599744515 | 0.8687887115787893 | 0.9034540074655802 |
| 270 | 0.8277663035490026 | 0.8692744042332281 | 0.8351363453457681 | 0.8793351372795405 | 0.8301864571739943 | 0.87913563 |
| 271 | 0.8727183152429779 | 0.9036958897609197 | 0.8654236023693469 | 0.9034845601880654 | 0.8703073132128429 | 0.9061290274548126 |
| 272 | 0.8835172762443987 | 0.9127437325543046 | 0.877557278 | 0.9129785720286593 | 0.8828324670980654 | 0.9156525595839067 |
| 273 | 0.8534203322908301 | 0.8880725905879675 | 0.8474175116184817 | 0.887031002 | 0.8449150123732865 | 0.8906504327121365 |
| 274 | 0.8793729996259702 | 0.9060882701373197 | 0.8717599064333903 | 0.9049356252273072 | 0.8745991542293784 | 0.9091531674331041 |
| 275 | 0.8832504378557653 | 0.9121994438562468 | 0.8759384756194301 | 0.910227129 | 0.8817105102385916 | 0.9140433386849356 |
| 276 | 0.8830430452728759 | 0.9106197403759104 | 0.875166905 | 0.9098947569071737 | 0.8781862113740545 | 0.913859126 |
| 277 | 0.7472506068582933 | 0.7979536069789911 | 0.7517220827592522 | 0.8063231627538621 | 0.7393814045131758 | 0.8048056456170983 |
| 278 | 0.8764558837327103 | 0.9056338601613045 | 0.8699478120349364 | 0.9067593143098552 | 0.8749819998773116 | 0.9100255646400679 |
| 279 | 0.8227327869610792 | 0.8650904289613991 | 0.8202665339344599 | 0.8663909193367334 | 0.8197313884445602 | 0.8714498892526694 |
| 280 | 0.8838888701879086 | 0.9116710298091816 | 0.8756497858740282 | 0.9102367151960256 | 0.880639476 | 0.9133204029658863 |
| 281 | 0.8857857206532282 | 0.9162524700502973 | 0.8779098126466736 | 0.9151327497727477 | 0.8853364775277147 | 0.917825724 |
| 282 | 0.8808892138528559 | 0.9102458454241141 | 0.8756328638402275 | 0.9100054682221952 | 0.8801251864714583 | 0.9141407660980723 |
| 283 | 0.8824056890748151 | 0.9102151034367681 | 0.8759061561901176 | 0.9092730741873504 | 0.8805368370780284 | 0.9130567543351943 |
| 284 | 0.5003938036955834 | 0.6320429244801712 | 0.5108786736000825 | 0.6293146359185466 | −0.465640548 | −0.607683554 |
| 285 | 0.7651436059034701 | 0.8362779308456737 | 0.7739189385503076 | 0.841962131 | 0.765794524 | 0.8424325090141931 |
| 286 | 0.8878066552631281 | 0.9155574439003897 | 0.8807312680803918 | 0.9148634778007187 | 0.8856512339154913 | 0.9181336227622454 |
| 287 | 0.8865343341795724 | 0.9136307339385025 | 0.878634028 | 0.9126940423754181 | 0.8838626797640964 | 0.9180226445839083 |
| 288 | 0.8802619539298241 | 0.909794292 | 0.8744972754698193 | 0.9079799377143518 | 0.8771152427723575 | 0.9125705719853093 |
| 289 | 0.8827604449908732 | 0.9116049452493585 | 0.8774289997378831 | 0.9103842209285603 | 0.8822729361037117 | 0.9149484277347188 |
| 290 | 0.8871331163547264 | 0.9144720205491507 | 0.881082362 | 0.9129205537043948 | 0.882719483 | 0.9163740748821378 |
| 291 | 0.8651094822985497 | 0.8966846339061674 | 0.8613323116562799 | 0.8946339543859443 | 0.8659387828704196 | 0.9022695133632275 |
| 292 | 0.8842314251777875 | 0.911360356 | 0.8775705790345779 | 0.9100765738541731 | 0.8840257232728116 | 0.9143437930560038 |
| 293 | 0.8844795598907026 | 0.9120486670962362 | 0.8780346448207673 | 0.9097390645565383 | 0.8829709239518089 | 0.9144770083870747 |
| 294 | 0.8641006063869007 | 0.8973330037977439 | 0.8582898372565004 | 0.8987416615831365 | 0.8609569760834123 | 0.9020525601933234 |
| 295 | 0.8792873542179493 | 0.9096381385655301 | 0.8732287834266913 | 0.908633575 | 0.8775322777490624 | 0.9132890311781109 |
| 296 | 0.8849701094705988 | 0.9128985944594831 | 0.8761187999984492 | 0.9106936714934228 | 0.8812894818134869 | 0.9137835836491252 |
| 297 | 0.8870317852307233 | 0.9139513882170942 | 0.8789499905411191 | 0.9140379796609279 | 0.8847830045200235 | 0.9181354271413481 |
| 298 | 0.8865308090350006 | 0.9121593737426633 | 0.8783849213858053 | 0.911410216 | 0.8830332134993616 | 0.9150891712342055 |
| 299 | 0.8844542619999826 | 0.9121812370851539 | 0.8767617694487385 | 0.9117142227467778 | 0.8818985719968497 | 0.9140628379035898 |
| 300 | 0.88406542 | 0.9103550191118321 | 0.8764647802664121 | 0.908471355 | 0.8812042706013552 | 0.9146973075451227 |
| 301 | 0.8855766478969509 | 0.9135542943725705 | 0.8786485695411894 | 0.9126877086899433 | 0.8817548880260155 | 0.917365406 |
| 302 | 0.8844251841512728 | 0.9127113410514476 | 0.8771218098147542 | 0.9104295986642046 | 0.8816020260265213 | 0.9155240455757966 |
| 303 | 0.8854784704306182 | 0.9132431807421235 | 0.8773053699276423 | 0.910712539 | 0.8828401453219171 | 0.9161838391783348 |
| 304 | 0.8579421338795843 | 0.8893025435636177 | 0.8525739090592342 | 0.8893712179181936 | 0.8583880018676904 | 0.8956831267605596 |
| 305 | 0.8006786483305603 | 0.8459083660877409 | 0.8076815884928225 | 0.8544204505644493 | 0.7953801094597326 | 0.8515092040902184 |
| 306 | 0.8701241978192478 | 0.9030666662394096 | 0.8639102717693005 | 0.9013950093253591 | 0.8678639133622885 | 0.9065475210406624 |
| 307 | 0.7259126681186532 | 0.7848936937954398 | 0.7346419587196458 | 0.7869098348202035 | 0.7050009380928327 | 0.7743919430952776 |
| 308 | 0.8079716862853596 | 0.8694368293892681 | 0.8161781610000804 | 0.8742490894019441 | 0.8064997593532472 | 0.8716956800078598 |
| 309 | 0.885480257 | 0.9135686116328973 | 0.879068567 | 0.9112373736131865 | 0.8828475538930176 | 0.9145790459539295 |
| 310 | 0.882419647 | 0.9100356052095125 | 0.8744020373137023 | 0.9087834590091086 | 0.8791704787598251 | 0.9137075081207446 |
| 311 | 0.8148178546702807 | 0.8619404000600389 | 0.8285930293988344 | 0.8715146165539812 | 0.8261133263022675 | 0.8809683831665204 |
| 312 | 0.8006194691463324 | 0.849487594 | 0.8071785010306068 | 0.8574342890006488 | 0.7994082033460662 | 0.8570699632303618 |
| 313 | 0.8879727458182349 | 0.9146114521365799 | 0.8801785960311038 | 0.9138359355959734 | 0.886112398 | 0.9172405792179048 |
| 314 | 0.7781545277793258 | 0.8266629826450108 | 0.7837698338972714 | 0.8345558312265013 | 0.7749795644826551 | 0.8334885774043266 |
| 315 | 0.8766584215518523 | 0.9074185065557356 | 0.8686115940714726 | 0.9036053679741886 | 0.8752920909844882 | 0.9114297772322397 |
| 316 | 0.8829130028653562 | 0.9132641389252238 | 0.8777296364540995 | 0.912595895 | 0.8801222893542395 | 0.9151977671925955 |
| 317 | 0.5552569601745182 | 0.6535296000113241 | 0.5692820996728296 | 0.6617699543486357 | 0.5095041370374684 | 0.6389568966319291 |
| 318 | 0.8863872194354009 | 0.916298574 | 0.8786935239289222 | 0.9130891999465182 | 0.885004598 | 0.9198011063487646 |
| 319 | −0.46705757 | −0.58993815 | −0.459194272 | −0.569178968 | 0.41557488031971357 | 0.5464349567954271 |
| 320 | 0.47401049039644927 | 0.6300000662195774 | 0.4414031419275021 | 0.599885277 | −0.454315938 | −0.623863751 |
| 321 | 0.8849866477311104 | 0.911561402 | 0.8781604457522104 | 0.9099090302467407 | 0.8837527489053645 | 0.9164777127445562 |
| 322 | 0.815365029 | 0.8607492390565753 | 0.8223435998924438 | 0.8671861134801911 | 0.822837322 | 0.8718181528615027 |
| 323 | 0.8855290779916011 | 0.9136742713294782 | 0.8781345997623854 | 0.9128961947179358 | 0.8811566569229042 | 0.9145158331631227 |
| 324 | 0.8312863239056473 | 0.8810556484264145 | 0.8309440217347088 | 0.8840946982086764 | 0.826147592 | 0.880923755 |
| 325 | 0.8839586897963942 | 0.9115062914925438 | 0.8772038908764233 | 0.9104058872482694 | 0.8818761519810878 | 0.9152336218820235 |
| 326 | 0.8775233985034822 | 0.9062125713654158 | 0.8712786856644978 | 0.9051718034505832 | 0.8763385638405744 | 0.9104477910773092 |
| 327 | 0.8790255840104007 | 0.90912197 | 0.8725043770502512 | 0.9081359404024805 | 0.8770477232506989 | 0.9126246503750186 |
| 328 | 0.8827378412630607 | 0.9121373559392846 | 0.8734574290034083 | 0.908984002 | 0.8764217449396023 | 0.9138001219924239 |
| 329 | 0.8868021951471156 | 0.9152342442490468 | 0.8793060719414163 | 0.9131320998759077 | 0.8853311397926937 | 0.9170900352350861 |
| 330 | 0.8863999429207874 | 0.9156585167966332 | 0.879752058 | 0.9141398488606145 | 0.8850163548984992 | 0.9189100552880958 |
| 331 | 0.8819547156686581 | 0.9126554681563678 | 0.8750625402669601 | 0.9114391235798636 | 0.8806060950746593 | 0.9157386003190884 |
| 332 | 0.8788923278124536 | 0.9061102088085174 | 0.8717906582188565 | 0.905505866 | 0.8776570474354352 | 0.9100289489221212 |
| 333 | 0.8586979401085408 | 0.8920000864850498 | 0.8580787963889648 | 0.8949165144508457 | 0.8612398907427945 | 0.9005263805726228 |
| 334 | 0.8809614486490416 | 0.9116508679672904 | 0.8745059595102344 | 0.9094212486910671 | 0.8784129533812562 | 0.9148045290321685 |
| 335 | 0.1689078855560262 | 0.2811996680269637 | 0.19922216465686748 | 0.28013624 | 0.13688160476190966 | 0.23201220033388661 |
| 336 | 0.5847728531393609 | 0.6998370481495393 | 0.6074404783418182 | 0.7127047559406828 | 0.5611367406576014 | 0.6985111487978082 |
| 337 | 0.881827398 | 0.909086937 | 0.8762146276888609 | 0.9092961563377124 | 0.8798539304373173 | 0.9133239270676976 |
| 338 | 0.8820752174683335 | 0.9104826646429891 | 0.8757862493449688 | 0.9096406248260663 | 0.8811978779952296 | 0.9141867585198739 |
| 339 | 0.7511538702032207 | 0.8267093488064217 | 0.7725343443448517 | 0.8371978169408889 | 0.7601637034828561 | 0.8349834280224766 |
| 340 | 0.8796426185179678 | 0.9085434338942364 | 0.8730134921230599 | 0.9078778597703522 | 0.8760870499282851 | 0.9115207284391525 |
| 341 | 0.8745968655789569 | 0.9059721837074293 | 0.8690846001808837 | 0.9063768568580749 | 0.8708010887574464 | 0.9092652372746435 |
| 342 | 0.778259352 | 0.8446348412731015 | 0.7885078728284838 | 0.8539109695625036 | 0.7779388039440757 | 0.8479658010287467 |
| 343 | 0.8877892784495272 | 0.9148703869210553 | 0.8812594623088457 | 0.9140974604098204 | 0.8863968404091348 | 0.9180574011121161 |
| 344 | 0.8732270230888657 | 0.9008446723525458 | 0.8637544459377537 | 0.8970892672384219 | 0.8707952393591949 | 0.9038433857995878 |
| 345 | 0.8827746469599446 | 0.9142964850787023 | 0.8757745014844442 | 0.9120366592020827 | 0.8814755110546805 | 0.9168743646689747 |
| 346 | 0.8851716877596816 | 0.9124647131139525 | 0.8791848506205997 | 0.9114875374583511 | 0.8830218177736331 | 0.9148100748153294 |
| 347 | 0.8817160751025024 | 0.9100546229778382 | 0.8741644955400775 | 0.9085121187576308 | 0.8780287361495325 | 0.9134241963050547 |
| 348 | 0.835932286 | 0.8767094239292588 | 0.8422211617714954 | 0.8833066086537174 | 0.8449446049029482 | 0.8902474926557502 |
| 349 | 0.8722713781175003 | 0.9033701411947365 | 0.8689420701758754 | 0.9053971114182671 | 0.8739655910915449 | 0.908985192 |
| 350 | 0.889298159 | 0.9153404732416732 | 0.8795654048453723 | 0.9144571304608782 | 0.8870678699776158 | 0.9173601656055164 |
| 351 | 0.8837756043956085 | 0.9103692092690513 | 0.8748810380381258 | 0.9081059850558931 | 0.8794765558731119 | 0.9134909740822188 |
| 352 | 0.8845959468873287 | 0.9100471807358121 | 0.876594516 | 0.9093235796013641 | 0.8792487904870407 | 0.9124745465029199 |
| 353 | 0.7633020927842065 | 0.8372860034216933 | 0.7887114758617917 | 0.8545857742435121 | 0.7717816949136072 | 0.8525886336615139 |
| 354 | 0.88570024 | 0.9137783605109489 | 0.8764269868426251 | 0.9117723819171208 | 0.8848224296909539 | 0.9174241743708805 |
| 355 | 0.829510535 | 0.8727856536125098 | 0.8356301556815084 | 0.8792599767648765 | 0.8355841079783939 | 0.883455842 |
| 356 | 0.8450602074300227 | 0.8852925827037882 | 0.8478218369973352 | 0.8896735381229128 | 0.8499489917920684 | 0.8946125434803356 |
| 357 | 0.826802189 | 0.869269634 | 0.8335590003441655 | 0.8744361736747237 | 0.8262321908145963 | 0.8757738249765997 |
| 358 | 0.8874604530597103 | 0.9159561757273682 | 0.8794347227934362 | 0.9158247766858372 | 0.8856743117306491 | 0.9198898589087847 |
| 359 | 0.7703658788366716 | 0.8170724316133926 | 0.7773250766005353 | 0.8233009438305936 | 0.7676088059459389 | 0.8255151585753905 |
| 360 | 0.8805347709226914 | 0.9095539162358979 | 0.8742691948927794 | 0.9086998253328575 | 0.8760537459775264 | 0.9127683175160469 |
| 361 | 0.7652649199065716 | 0.8296587679600987 | 0.7754052628140068 | 0.8345241224799917 | 0.760818081 | 0.8290357590038231 |
| 362 | 0.8814227036906049 | 0.9080578324117013 | 0.8733434163184723 | 0.9060018720045124 | 0.8783661076774836 | 0.9110605179448517 |
| 363 | 0.8833812057058474 | 0.9122416357475895 | 0.8767737389183635 | 0.9118932331704522 | 0.8827840677500669 | 0.9149058670606456 |
| 364 | 0.8881827135186322 | 0.915750734 | 0.8808951840696098 | 0.9139994443895645 | 0.8864544398305136 | 0.9191460149561953 |
| 365 | 0.8874576856898944 | 0.9151050417548535 | 0.8791850576642879 | 0.9136147927405889 | 0.885311648 | 0.9170549235304177 |
| 366 | 0.8825267536578709 | 0.9097640310426105 | 0.8750451818624636 | 0.9066357583868212 | 0.8792220321278374 | 0.9120941455436374 |
| 367 | 0.8860277525085362 | 0.9144168737527206 | 0.8782985277805159 | 0.9128791148343727 | 0.883143835 | 0.9154922749416986 |
| 368 | 0.8407893545854085 | 0.8804362926999093 | 0.8444836754822738 | 0.8854585282979338 | 0.8449025486850357 | 0.8897775719062797 |
| 369 | 0.7324913798417804 | 0.7886265897362178 | 0.7417237364011222 | 0.7939680283274464 | 0.7283248508783966 | 0.7943308716035525 |
| 370 | 0.8864577188805829 | 0.9143022881227914 | 0.8776690186502375 | 0.9143044822743127 | 0.8854049397288014 | 0.9177492448122472 |
| 371 | 0.7159530446275689 | 0.7758587897697479 | 0.7304465754335773 | 0.7891950848654248 | 0.706470497 | 0.7796048615534502 |
| 372 | 0.8845924001793802 | 0.911790008 | 0.8771485023655454 | 0.9088978075073194 | 0.882788597 | 0.9136408362457167 |
| 373 | 0.878310604 | 0.9071883720026541 | 0.8710114705634409 | 0.9052033820733452 | 0.8737546736299733 | 0.9093251134202215 |
| 374 | 0.8723683841576253 | 0.9038517037802344 | 0.8658575215548248 | 0.9051339245732708 | 0.868990646 | 0.9077686598262812 |
| 375 | 0.8830723507107745 | 0.9112609594054992 | 0.8749487677839182 | 0.9090615762004846 | 0.8789299725219816 | 0.9133204762524456 |
| 376 | 0.8344119929199776 | 0.8764032561317645 | 0.8372458422425753 | 0.8804640301854272 | 0.8341810783020773 | 0.8845364483156044 |
| 377 | 0.8516881780751463 | 0.8900409492234261 | 0.8552084730887974 | 0.8944765046248735 | 0.8573753867649379 | 0.9004692116221679 |
| 378 | 0.8863051185030475 | 0.9149601853806079 | 0.880791506 | 0.9150049305349509 | 0.8859939710356707 | 0.9198139506171429 |
| 379 | 0.8250439236198439 | 0.8670149073393537 | 0.8239591086601774 | 0.867235588 | 0.8194916974239467 | 0.8692262273179125 |
| 380 | 0.6625032337910464 | 0.7506003089511346 | 0.6784536139644681 | 0.7597569599153786 | 0.6377368580821018 | 0.7514005010829421 |
| 381 | 0.8881035810046187 | 0.9137854849301228 | 0.877903178 | 0.9125309524711991 | 0.8851867646749754 | 0.9175078813552443 |
| 382 | 0.8729151252262589 | 0.9034916148859606 | 0.867219879 | 0.9049977238648351 | 0.8707088945539753 | 0.9085353118656794 |
| 383 | 0.8657152003368288 | 0.89702295 | 0.862319843 | 0.9010402365338079 | 0.8643691125470838 | 0.9040292374312149 |
| 384 | 0.8726117120781058 | 0.9025392519151336 | 0.8675027898999476 | 0.9032549104125835 | 0.8740984740901299 | 0.9086277459442558 |
| 385 | 0.8810467576320516 | 0.9097009595470751 | 0.8733823004792735 | 0.9080883818655153 | 0.8787334111981022 | 0.9128125749226795 |
| 386 | 0.8678169382773084 | 0.9019781853085981 | 0.8619906380831173 | 0.9016794784926686 | 0.866351245 | 0.9054478816283145 |
| 387 | 0.8861528321667734 | 0.9132600352724349 | 0.8785438363548898 | 0.9128355925957511 | 0.8833856610621215 | 0.9181736951918469 |
| 388 | 0.8232587324431583 | 0.8662245274974079 | 0.8304782390287055 | 0.8754211774018512 | 0.8264464065703306 | 0.8786604398324727 |
| 389 | 0.8062352639699115 | 0.856165953 | 0.828946245 | 0.8724927112684374 | 0.8296329618857002 | 0.8808195418435735 |
| 390 | 0.7118623539607715 | 0.7846640009317731 | 0.7263018094151839 | 0.7941812099122644 | 0.6978563282046435 | 0.7889027636275872 |
| 391 | 0.733521645 | 0.7896529317223473 | 0.7487849138466038 | 0.7997988330236083 | 0.7387119067527639 | 0.7922059541837331 |
| 392 | 0.8851174416053432 | 0.9147379608357309 | 0.8777281023705127 | 0.913826393 | 0.880725202 | 0.9156982299480517 |
| 393 | 0.886799016 | 0.9143898284189942 | 0.8797592198745456 | 0.9139150065419952 | 0.885711159 | 0.9175676801280683 |
| 394 | 0.8818332712095664 | 0.9072537678760901 | 0.8724707726709586 | 0.9042500593865902 | 0.8783773491770591 | 0.9111451274146714 |
| 395 | 0.8861356080940338 | 0.9119794167114887 | 0.8800008384853018 | 0.9110251554996708 | 0.8836444294513089 | 0.9160891565169675 |
| 396 | 0.7908284215654378 | 0.8547704210949405 | 0.8050570955752983 | 0.8648586161554335 | 0.7918818782710378 | 0.8629640678840148 |
| 397 | 0.8887857887194782 | 0.9156384883234889 | 0.8793528161391582 | 0.9148590885733378 | 0.8842143904446114 | 0.9180860287442107 |
| 398 | 0.8477516611181972 | 0.8867959648361046 | 0.8464803833386798 | 0.8901041076009051 | 0.8484621139004895 | 0.8934231748185176 |
| 399 | 0.8755860525077639 | 0.9067176962325917 | 0.869882502 | 0.9052965238559944 | 0.8730749981952187 | 0.9086724906723527 |
| 400 | 0.883853776 | 0.9140684345816091 | 0.876525582 | 0.913107652 | 0.8834648501619071 | 0.9165053387051436 |
| 401 | 0.8827883683884208 | 0.9140072315030983 | 0.8767803626249795 | 0.9141100512382335 | 0.8830797988077049 | 0.9182198296168969 |
| 402 | 0.8198902864738179 | 0.8701602343969043 | 0.8250151458259588 | 0.8772466673492102 | 0.8257729083387758 | 0.8746343066117811 |
| 403 | 0.8794111219221497 | 0.9100634144606317 | 0.8728622435165828 | 0.9109334123529826 | 0.8755726566342704 | 0.9140473200064216 |
| 404 | −0.401110309 | −0.514300943 | −0.423591713 | −0.550090088 | 0.4312671936236627 | 0.5682720126755452 |
| 405 | 0.8835898843165205 | 0.9103811910770628 | 0.876576535 | 0.9100950952549441 | 0.8802992653521831 | 0.9139396199043954 |
| 406 | 0.885721929 | 0.9146947160768458 | 0.8771318339016494 | 0.913421209 | 0.883317613 | 0.9161229673099507 |
| 407 | 0.8801409682009034 | 0.9075994596121378 | 0.8733885993181709 | 0.9063944229665009 | 0.876165712 | 0.9103336077965523 |
| 408 | 0.8848993024025886 | 0.9139298478766908 | 0.8779699232800533 | 0.9126953172209056 | 0.8850837472866064 | 0.9178479223748941 |
| 409 | 0.8862508794591144 | 0.9117450333966768 | 0.8780858467618244 | 0.9092061204929005 | 0.8840596101917821 | 0.9149965898650798 |
| 410 | 0.8903794814518727 | 0.9171284186172282 | 0.8818818996739721 | 0.9156756522454311 | 0.8865353911531901 | 0.920921654 |
| 411 | 0.8640228550719335 | 0.899110682 | 0.8587831466174312 | 0.9006069021130475 | 0.8663432346960875 | 0.90282497 |
| 412 | 0.777745482 | 0.8421614718320901 | 0.7880006530726167 | 0.8507070117837382 | 0.7796871401403146 | 0.850558308 |
| 413 | 0.8357428895659729 | 0.8759506934546107 | 0.8407188699988761 | 0.8787370633243291 | 0.8401898708069849 | 0.8840664934663036 |
| 414 | 0.8858051729208496 | 0.9133527303759266 | 0.8769266172073868 | 0.9118098753006132 | 0.8815417783240542 | 0.9152873765803257 |
| 415 | 0.8757654595155552 | 0.9075770289397469 | 0.8706093897810049 | 0.9054984796043222 | 0.8741754138720563 | 0.9085768292154652 |
| 416 | 0.8835934492124089 | 0.9108902037087016 | 0.8774734840941767 | 0.9092717336098629 | 0.8818483764651572 | 0.9135887044510714 |
| 417 | 0.8865075604699487 | 0.9149492895805904 | 0.878859579 | 0.9127442487547154 | 0.8829065055043905 | 0.9160145124783279 |
| 418 | 0.8791101621664499 | 0.9087398970727789 | 0.8726473144802507 | 0.9067501187760643 | 0.8791479835296173 | 0.9124014616398302 |
| 419 | 0.8882187851916687 | 0.9162688780813537 | 0.8819695164630561 | 0.914996553 | 0.8866031165758745 | 0.9185190774237251 |
| 420 | 0.8846947987481593 | 0.9127817794765881 | 0.8768311559998784 | 0.9115792194848571 | 0.8809000994621996 | 0.9148005064473329 |
| 421 | 0.8835809834066837 | 0.9112160949070376 | 0.8762058200931465 | 0.9105855645397809 | 0.8801213208307852 | 0.9134496616794555 |
| 422 | 0.8822402585353686 | 0.9108316276021221 | 0.8753854269419115 | 0.9107412476371493 | 0.8792447062279048 | 0.9144842857562954 |
| 423 | 0.8875392243328725 | 0.9140189791048273 | 0.8802253215427933 | 0.9114564740634573 | 0.8842672834800411 | 0.9160630028676886 |
| 424 | 0.8845673496271219 | 0.9122453252108841 | 0.8774712075210838 | 0.9104632624304438 | 0.8823483165377529 | 0.9144975776984899 |
| 425 | 0.8704949287333833 | 0.9036862786305584 | 0.8652293893525095 | 0.9020625416490964 | 0.8674060459343466 | 0.9070012239571029 |
| 426 | 0.8840974163193394 | 0.9089109030213743 | 0.8766034905034695 | 0.9080439161913836 | 0.8806817823920766 | 0.9112695254760107 |
| 427 | 0.8867306247104293 | 0.9140087824495319 | 0.8798491365995094 | 0.9139130831293792 | 0.8850798643123877 | 0.9184040819019705 |
| 428 | 0.8221191830822894 | 0.8633910044592227 | 0.8214641740472405 | 0.8682296542750996 | 0.8223495251926829 | 0.8693584152815704 |
| 429 | 0.8870001607213771 | 0.9134098169828616 | 0.8795569128652913 | 0.9120119700869235 | 0.8843557831096861 | 0.9173697674456914 |
| 430 | 0.8622699321148386 | 0.8922475393808548 | 0.8600132463561517 | 0.8927518913894449 | 0.8648122020068634 | 0.9005654329295428 |
| 431 | 0.8843101941957388 | 0.9129314587145068 | 0.8762404658561995 | 0.9121043057034148 | 0.8810266849376641 | 0.9147530592440721 |
| 432 | 0.856934033 | 0.8919867615594704 | 0.8562390667381933 | 0.8982189375645632 | 0.8588728777880289 | 0.8984865296673751 |
| 433 | 0.8624523779733546 | 0.8953718724522017 | 0.8603822969460934 | 0.8972651373819461 | 0.8622515971684088 | 0.9017906545156177 |
| 434 | 0.42813226840766405 | 0.5607614964052766 | 0.4446386208477283 | 0.5715158332735699 | 0.412285376 | 0.5664744214165827 |
| 435 | 0.8230877778789611 | 0.8669791706262017 | 0.8311634538140643 | 0.8748978707353727 | 0.831746413 | 0.8816545483750627 |
| 436 | 0.8879622200331714 | 0.9162657226956598 | 0.8811811683044617 | 0.914638409 | 0.8873400881763208 | 0.9183918843916666 |
| 437 | 0.886075087 | 0.914319765 | 0.8793017631722854 | 0.9136659003083082 | 0.8835421866966937 | 0.9178699607596463 |
| 438 | 0.8881424766710512 | 0.9148506114001018 | 0.8771516256393279 | 0.9136671361580705 | 0.883604864 | 0.9170014664663424 |
| 439 | 0.8517225665442685 | 0.8849752790612886 | 0.8494501611404108 | 0.885993992 | 0.8496784309515075 | 0.8889463054881933 |
| 440 | 0.8831079170357218 | 0.9111750492736238 | 0.8765999454288533 | 0.9103393598400322 | 0.8792717597218679 | 0.9142040738579522 |
| 441 | 0.8850547326205991 | 0.9130772552781304 | 0.8775266688115577 | 0.9105899143512035 | 0.883135376 | 0.9164665695457328 |
| 442 | 0.3021014400869836 | 0.39367141454696636 | 0.41283374079727203 | 0.5336939651889082 | 0.37095181508198916 | 0.5301915198253337 |
| 443 | 0.8851135998585726 | 0.9113388509665723 | 0.8771958049048694 | 0.9112172523477848 | 0.8814142118529371 | 0.9145108958427468 |
| 444 | 0.8838584527146653 | 0.9104952747588531 | 0.8748698406999048 | 0.9071956759831509 | 0.8766196893931969 | 0.9114285318600788 |
| 445 | 0.8868045330218755 | 0.9148221843540755 | 0.8781610054443758 | 0.9141089116342533 | 0.8836515669612918 | 0.9181313159258961 |
| 446 | 0.8844716464489635 | 0.9107072738303273 | 0.8762066274418587 | 0.908555217 | 0.8811982825643665 | 0.9129190634657947 |
| 447 | 0.8872785771615785 | 0.9146521207650143 | 0.8800732591172501 | 0.91439569 | 0.8852313405593492 | 0.9171591743341598 |
| 448 | 0.882555408 | 0.9123248914698519 | 0.8759494937184743 | 0.911015359 | 0.8828006462500584 | 0.9161716876205559 |
| 449 | 0.8840550391008719 | 0.912974428 | 0.8759589699554446 | 0.9127224569078114 | 0.8784681699558538 | 0.9160938417483323 |
| 450 | 0.8897205912646098 | 0.9171364613522912 | 0.8802607976828849 | 0.9151908734782316 | 0.8901061809617571 | 0.9201692977870897 |
| 451 | 0.7675461724274757 | 0.8162660818052221 | 0.7756715825154823 | 0.8192842368563489 | 0.7580138712582081 | 0.8173521213924357 |
| 452 | 0.886073622 | 0.9141471521129035 | 0.8788800636317738 | 0.9123296796102097 | 0.8822403528505378 | 0.9159771840655355 |
| 453 | 0.857401676 | 0.8917593709458775 | 0.8560284022490681 | 0.8925842446633419 | 0.8553495466250342 | 0.8970319181696317 |
| 454 | 0.8805337468385102 | 0.9101523012040568 | 0.8732594087159471 | 0.9078618397110915 | 0.8775258835031228 | 0.9123454734188519 |
| 455 | 0.8832400052308814 | 0.9107243904436169 | 0.8771072405946101 | 0.9095195070537888 | 0.880995356 | 0.9151326182999825 |
| 456 | 0.8856258370254226 | 0.9157276354636001 | 0.8783295866200794 | 0.9138404118908946 | 0.8820921493819869 | 0.916643828 |
| 457 | 0.7480836652549466 | 0.8214699085159218 | 0.7696626359903559 | 0.8298467644251556 | 0.7535229315522338 | 0.828758567 |
| 458 | 0.8876317337101103 | 0.9138082432790593 | 0.8800338218549016 | 0.9113454491772338 | 0.884795311 | 0.9158079297242498 |
| 459 | 0.8876235266771864 | 0.9154777894761328 | 0.8788655400904462 | 0.9149489466178988 | 0.8849377095418277 | 0.9186724907282088 |
| 460 | 0.7556996267792003 | 0.8012790283457244 | 0.7592851873559533 | 0.8058364871746961 | 0.7414160617608047 | 0.8008673322293959 |
| 461 | 0.8852900508435476 | 0.9125578493472862 | 0.8785318575458221 | 0.911418924 | 0.8830613812787346 | 0.9147618703393746 |
| 462 | 0.8848052456647704 | 0.9132322327501794 | 0.8778674945159273 | 0.9121693454231694 | 0.8817585495329698 | 0.9154184167770825 |
| 463 | 0.8774257145503105 | 0.9086539197547356 | 0.8711109626335549 | 0.907693613 | 0.8749509218257207 | 0.9112012000604742 |
| 464 | 0.8803983662561984 | 0.9117245362521204 | 0.8736019191506124 | 0.910674349 | 0.8785328794869014 | 0.9143292004020336 |
| 465 | 0.8860702760176924 | 0.9120220030371258 | 0.8783389107363317 | 0.9104191162661246 | 0.8825788588403832 | 0.9142135973243012 |
| 466 | 0.8773421980150112 | 0.9068924224444712 | 0.8708201840240855 | 0.9054273998043214 | 0.8749237540487823 | 0.9116786593405716 |
| 467 | 0.88750849 | 0.9155506462967762 | 0.8796606986185767 | 0.9140835389332812 | 0.885470288 | 0.9187492408032335 |
| 468 | 0.8832316217939273 | 0.9099467969065538 | 0.8758770361921495 | 0.9091156602326218 | 0.878578981 | 0.912868215 |
| 469 | 0.8537493979126469 | 0.8910729025857734 | 0.8517005922974613 | 0.8921597420581181 | 0.8556011842687749 | 0.8985562717208544 |
| 470 | 0.8693327741917347 | 0.9033032280991314 | 0.8644392098184932 | 0.9026057238322289 | 0.8700438304956728 | 0.9073524445962968 |
| 471 | 0.8336077870673222 | 0.8644313562712147 | 0.838938169 | 0.8754644193269066 | | |
| 472 | 0.8850635110973422 | 0.9134160568632554 | 0.877484153 | 0.9116340883889994 | 0.8834866240974607 | 0.9154763496741622 |
| 473 | 0.8768008020206072 | 0.903705672 | 0.8710534392154493 | 0.9022400920834864 | 0.8751718966725922 | 0.909518439 |
| 474 | 0.8852821041467094 | 0.912581375 | 0.8776914589326034 | 0.9112261205047656 | 0.8814667289245686 | 0.9159029259727094 |
| 475 | 0.8744140752111207 | 0.9046288593608717 | 0.8683229510788705 | 0.9033744803182642 | 0.8733999990641612 | 0.9090835255700193 |
| 476 | 0.8666130779082835 | 0.9004531075317141 | 0.8601873917129662 | 0.9011248568177839 | 0.8664982227802756 | 0.9065608406023585 |
| 477 | 0.8811440500095413 | 0.91079859 | 0.8746017693694534 | 0.9087741051210763 | 0.8767607999177562 | 0.9120953043235684 |
| 478 | 0.8727306714782964 | 0.9044833550635365 | 0.8657716841105152 | 0.9050021258721328 | 0.871271684 | 0.910259308 |
| 479 | 0.7797980377676265 | 0.8479619610780885 | 0.7925191827342312 | 0.8580052820355879 | 0.7832209327205985 | 0.856327666 |
| 480 | 0.8779631020600448 | 0.907479646 | 0.8723812963666931 | 0.9067901447143394 | 0.8738770557489113 | 0.910215325 |
| 481 | 0.8187709086526347 | 0.8666795267948308 | 0.8268890422482924 | 0.8727727443423305 | 0.8245235548165114 | 0.8773499988003206 |
| 482 | 0.7891183760126173 | 0.833526009 | 0.7952424270180543 | 0.8402794565737406 | 0.7878431385707425 | 0.8383408105851534 |
| 483 | 0.8149004026463271 | 0.8711273459829907 | 0.8218548991297374 | 0.8740135821272063 | 0.8135170273904506 | 0.8732924692409566 |
| 484 | 0.8644685144866108 | 0.8976374432502819 | 0.8597058547973548 | 0.8948008981760369 | 0.8572375887090089 | 0.8996277021307623 |
| 485 | 0.8856521293452432 | 0.9135765561336308 | 0.8790284532506751 | 0.9124410359848739 | 0.8830935572688705 | 0.9163392662661097 |
| 486 | 0.8842080492138695 | 0.9114642987559467 | 0.8768770873416664 | 0.9093789942832207 | 0.8822267652170168 | 0.9141272124522288 |
| 487 | 0.8784682447803036 | 0.9074343830638169 | 0.8703869217030854 | 0.9038575410260561 | 0.8741469956931534 | 0.9092197036938157 |
| 488 | 0.7993349408195423 | 0.8603439188336934 | 0.8033401434986405 | 0.8654099574411245 | 0.7907018273646954 | 0.8612095435190443 |
| 489 | 0.8825728787959366 | 0.9137824727376409 | 0.8765834008599213 | 0.91350804 | 0.8802265658016618 | 0.9159747105459384 |
| 490 | 0.883341617 | 0.9108701215108055 | 0.8755802328641179 | 0.9096299694391883 | 0.8818764232552851 | 0.9136892495566904 |
| 491 | 0.8714384493519755 | 0.9007281551946958 | 0.8673668699601663 | 0.9009719838765868 | 0.8672436530827256 | 0.9045238146772994 |
| 492 | 0.8638242505652202 | 0.8992516466404433 | 0.8622216898171036 | 0.9007416673015616 | 0.8660299015120706 | 0.9056632544542278 |

| Row ID | batch_size | padded_seq_len | duplication_cutoff | use_reverse_complements | input_len | conv1_channels |
|---|---|---|---|---|---|---|
| 1 | 597 | 600 | 2.585933251919139 | TRUE | 600 | 300 |
| 2 | 1078 | 600 | 5 | TRUE | 600 | 300 |
| 3 | 1653 | 216 | 0.5001945393938663 | TRUE | 216 | 2045 |
| 4 | 282 | 600 | 3.815109304417796 | TRUE | 600 | 300 |
| 5 | 853 | 600 | 5 | TRUE | 600 | 300 |
| 6 | 561 | 600 | 5 | TRUE | 600 | 300 |
| 7 | 281 | 216 | 2.7454392114716346 | FALSE | 216 | 386 |
| 8 | 838 | 600 | 4.981160405731163 | TRUE | 600 | 300 |
| 9 | 1175 | 216 | 2.785568563088108 | TRUE | 216 | 404 |
| 10 | 842 | 600 | 4.999700554937823 | TRUE | 600 | 300 |
| 11 | 961 | 600 | 4.941710859726525 | TRUE | 600 | 300 |
| 12 | 727 | 600 | 4.874908836556234 | TRUE | 600 | 300 |
| 13 | 279 | 600 | 3.833654543969396 | TRUE | 600 | 300 |
| 14 | 302 | 216 | 1.6131032412469506 | TRUE | 216 | 799 |
| 15 | 588 | 600 | 4.089524615894734 | TRUE | 600 | 300 |
| 16 | 1011 | 600 | 4.690770772565839 | TRUE | 600 | 300 |
| 17 | 801 | 600 | 5 | TRUE | 600 | 300 |
| 18 | 320 | 600 | 3.553449280190897 | FALSE | 600 | 300 |
| 19 | 898 | 600 | 4.81141737 | TRUE | 600 | 300 |
| 20 | 831 | 600 | 5 | TRUE | 600 | 300 |
| 21 | 738 | 600 | 4.957800668 | TRUE | 600 | 300 |
| 22 | 1019 | 600 | 4.978798126374305 | TRUE | 600 | 300 |
| 23 | 1154 | 216 | 3.157772257 | TRUE | 216 | 810 |
| 24 | 835 | 600 | 4.670920291670139 | TRUE | 600 | 300 |
| 25 | 1190 | 600 | 5 | TRUE | 600 | 300 |
| 26 | 381 | 600 | 3.3618274450389167 | TRUE | 600 | 300 |
| 27 | 415 | 600 | 3.429858100361803 | TRUE | 600 | 300 |
| 28 | 740 | 600 | 4.997607633 | TRUE | 600 | 300 |
| 29 | 749 | 600 | 3.6134315521983664 | TRUE | 600 | 300 |
| 30 | 573 | 600 | 4.547884615201551 | TRUE | 600 | 300 |
| 31 | 506 | 600 | 3.747011399755711 | TRUE | 600 | 300 |
| 32 | 748 | 600 | 5 | TRUE | 600 | 300 |
| 33 | 573 | 216 | 2.7953308005478505 | TRUE | 216 | 152 |
| 34 | 613 | 600 | 5 | TRUE | 600 | 300 |
| 35 | 820 | 600 | 5 | TRUE | 600 | 300 |
| 36 | 385 | 216 | 4.99380867 | FALSE | 216 | 428 |
| 37 | 854 | 600 | 4.973498613084123 | TRUE | 600 | 300 |
| 38 | 1326 | 216 | 0.5 | TRUE | 216 | 2048 |
| 39 | 600 | 600 | 4.950009671783063 | TRUE | 600 | 300 |
| 40 | 439 | 600 | 2.758406722594748 | FALSE | 600 | 300 |
| 41 | 1542 | 216 | 0.5 | TRUE | 216 | 2034 |
| 42 | 822 | 600 | 5 | TRUE | 600 | 300 |
| 43 | 1002 | 216 | 0.6125038261518716 | FALSE | 216 | 2048 |
| 44 | 901 | 600 | 4.994840852431167 | TRUE | 600 | 300 |
| 45 | 780 | 216 | 3.064987980775413 | FALSE | 216 | 119 |
| 46 | 674 | 216 | 3.4588159945562618 | TRUE | 216 | 361 |
| 47 | 709 | 600 | 4.636706437705137 | TRUE | 600 | 300 |
| 48 | 425 | 600 | 3.772643686534841 | TRUE | 600 | 300 |
| 49 | 908 | 600 | 4.797583138673221 | TRUE | 600 | 300 |
| 50 | 713 | 600 | 2.6335253922252257 | TRUE | 600 | 300 |
| 51 | 1402 | 216 | 0.5 | TRUE | 216 | 1651 |
| 52 | 723 | 600 | 4.989067632167879 | TRUE | 600 | 300 |
| 53 | 842 | 600 | 5 | TRUE | 600 | 300 |
| 54 | 686 | 600 | 5 | TRUE | 600 | 300 |
| 55 | 3071 | 600 | 0.5002567983413346 | TRUE | 600 | 300 |
| 56 | 703 | 216 | 2.620953847 | TRUE | 216 | 472 |
| 57 | 572 | 600 | 0.9695679312047828 | TRUE | 600 | 300 |
| 58 | 729 | 600 | 4.890314998473463 | TRUE | 600 | 300 |
| 59 | 964 | 600 | 5 | TRUE | 600 | 300 |
| 60 | 1018 | 600 | 4.379636639194498 | TRUE | 600 | 300 |
| 61 | 520 | 600 | 5 | TRUE | 600 | 300 |
| 62 | 1106 | 600 | 2.923038526933244 | FALSE | 600 | 300 |
| 63 | 1070 | 600 | 4.577723574469978 | TRUE | 600 | 300 |
| 64 | 911 | 600 | 5 | TRUE | 600 | 300 |
| 65 | 550 | 600 | 4.527418812184491 | TRUE | 600 | 300 |
| 66 | 552 | 600 | 4.147409478969409 | TRUE | 600 | 300 |
| 67 | 654 | 600 | 5 | TRUE | 600 | 300 |
| 68 | 609 | 600 | 5 | TRUE | 600 | 300 |
| 69 | 1693 | 600 | 5 | TRUE | 600 | 300 |
| 70 | 829 | 600 | 5 | TRUE | 600 | 300 |
| 71 | 814 | 600 | 4.893448659359432 | TRUE | 600 | 300 |
| 72 | 1861 | 216 | 0.5401845527028297 | TRUE | 216 | 1657 |
| 73 | 974 | 216 | 3.330537711874809 | TRUE | 216 | 126 |
| 74 | 771 | 216 | 4.942105384069814 | TRUE | 216 | 259 |
| 75 | 971 | 600 | 5 | TRUE | 600 | 300 |
| 76 | 1045 | 600 | 4.966283573388623 | TRUE | 600 | 300 |
| 77 | 136 | 216 | 3.057824390404506 | TRUE | 216 | 257 |
| 78 | 1147 | 600 | 4.617251146735552 | TRUE | 600 | 300 |
| 79 | 872 | 216 | 3.018220565668408 | FALSE | 216 | 2027 |
| 80 | 822 | 600 | 5 | TRUE | 600 | 300 |
| 81 | 940 | 600 | 4.694147800061571 | TRUE | 600 | 300 |
| 82 | 1210 | 600 | 4.144106660452646 | TRUE | 600 | 300 |
| 83 | 578 | 600 | 4.993985999874193 | TRUE | 600 | 300 |
| 84 | 1193 | 216 | 0.5976544751294006 | TRUE | 216 | 928 |
| 85 | 931 | 600 | 4.952392958 | TRUE | 600 | 300 |
| 86 | 587 | 600 | 4.992794354750044 | TRUE | 600 | 300 |
| 87 | 998 | 600 | 4.999714272618476 | TRUE | 600 | 300 |
| 88 | 837 | 600 | 5 | TRUE | 600 | 300 |
| 89 | 774 | 600 | 4.99834823 | TRUE | 600 | 300 |
| 90 | 627 | 600 | 2.75 | FALSE | 600 | 300 |
| 91 | 480 | 216 | 2.6790944222076005 | TRUE | 216 | 223 |
| 92 | 808 | 600 | 5 | TRUE | 600 | 300 |
| 93 | 730 | 600 | 5 | TRUE | 600 | 300 |
| 94 | 1486 | 600 | 5 | TRUE | 600 | 300 |
| 95 | 2999 | 600 | 5 | TRUE | 600 | 300 |
| 96 | 1145 | 216 | 2.069884019 | FALSE | 216 | 1140 |
| 97 | 712 | 600 | 4.9698343297827385 | TRUE | 600 | 300 |
| 98 | 799 | 600 | 4.846592852679191 | TRUE | 600 | 300 |
| 99 | 874 | 600 | 4.998892674140092 | TRUE | 600 | 300 |
| 100 | 1334 | 600 | 4.999791859973659 | TRUE | 600 | 300 |
| 101 | 620 | 600 | 4.792328318916778 | TRUE | 600 | 300 |
| 102 | 1237 | 600 | 4.567492879341553 | TRUE | 600 | 300 |
| 103 | 1046 | 600 | 4.506281374758976 | TRUE | 600 | 300 |
| 104 | 526 | 600 | 4.382859280195694 | TRUE | 600 | 300 |
| 105 | 937 | 600 | 5 | TRUE | 600 | 300 |
| 106 | 713 | 600 | 5 | TRUE | 600 | 300 |
| 107 | 806 | 600 | 4.994725057503162 | TRUE | 600 | 300 |
| 108 | 1436 | 600 | 5 | TRUE | 600 | 300 |
| 109 | 1020 | 600 | 4.200577288329518 | FALSE | 600 | 300 |
| 110 | 1864 | 600 | 0.5274777675335236 | TRUE | 600 | 300 |
| 111 | 988 | 600 | 4.2861104951327365 | TRUE | 600 | 300 |
| 112 | 844 | 600 | 5 | FALSE | 600 | 300 |
| 113 | 708 | 600 | 3.443871353820903 | TRUE | 600 | 300 |
| 114 | 864 | 600 | 4.9416892933 | TRUE | 600 | 300 |
| 115 | 876 | 600 | 5 | TRUE | 600 | 300 |
| 116 | 841 | 600 | 5 | TRUE | 600 | 300 |
| 117 | 276 | 600 | 3.211471567872478 | TRUE | 600 | 300 |
| 118 | 592 | 600 | 4.949732137866485 | TRUE | 600 | 300 |
| 119 | 961 | 600 | 4.316303106095663 | TRUE | 600 | 300 |
| 120 | 903 | 600 | 3.2507911867348187 | FALSE | 600 | 300 |
| 121 | 1014 | 600 | 2.6308362093460964 | TRUE | 600 | 300 |
| 122 | 567 | 600 | 4.943921099771269 | TRUE | 600 | 300 |
| 123 | 702 | 600 | 4.990684941734229 | TRUE | 600 | 300 |
| 124 | 728 | 600 | 5 | TRUE | 600 | 300 |
| 125 | 821 | 600 | 5 | TRUE | 600 | 300 |
| 126 | 1046 | 600 | 5 | TRUE | 600 | 300 |
| 127 | 841 | 600 | 5 | TRUE | 600 | 300 |
| 128 | 884 | 600 | 5 | TRUE | 600 | 300 |
| 129 | 989 | 600 | 4.588491609011296 | TRUE | 600 | 300 |
| 130 | 882 | 600 | 3.118441952204173 | TRUE | 600 | 300 |
| 131 | 766 | 600 | 5 | TRUE | 600 | 300 |
| 132 | 639 | 600 | 5 | TRUE | 600 | 300 |
| 133 | 1118 | 600 | 4.545210947258029 | TRUE | 600 | 300 |
| 134 | 802 | 600 | 4.840907905255451 | TRUE | 600 | 300 |
| 135 | 636 | 216 | 2.518673328742951 | TRUE | 216 | 245 |
| 136 | 254 | 600 | 3.267297263125552 | TRUE | 600 | 300 |
| 137 | 2705 | 216 | 2.5606876927498066 | TRUE | 216 | 64 |
| 138 | 322 | 216 | 0.581689347 | TRUE | 216 | 1000 |
| 139 | 768 | 216 | 1.060740445565917 | TRUE | 216 | 1243 |
| 140 | 538 | 216 | 2.3337366463335334 | TRUE | 216 | 249 |
| 141 | 838 | 600 | 4.9981681709208985 | TRUE | 600 | 300 |
| 142 | 1011 | 600 | 4.6639350271020215 | TRUE | 600 | 300 |
| 143 | 598 | 216 | 3.0484556410902943 | TRUE | 216 | 957 |
| 144 | 898 | 600 | 5 | TRUE | 600 | 300 |
| 145 | 717 | 600 | 4.995676016359573 | TRUE | 600 | 300 |
| 146 | 128 | 216 | 2.375646473277874 | FALSE | 216 | 565 |
| 147 | 989 | 600 | 5 | TRUE | 600 | 300 |
| 148 | 946 | 216 | 0.5 | TRUE | 216 | 360 |
| 149 | 648 | 600 | 3.496266131141591 | TRUE | 600 | 300 |
| 150 | 556 | 600 | 3.221834647357455 | TRUE | 600 | 300 |
| 151 | 706 | 600 | 4.843666121 | TRUE | 600 | 300 |
| 152 | 2097 | 216 | 0.5 | FALSE | 216 | 1442 |
| 153 | 668 | 600 | 4.956323574167541 | TRUE | 600 | 300 |
| 154 | 1509 | 216 | 0.5 | TRUE | 216 | 2048 |
| 155 | 1452 | 216 | 0.5007913172807241 | TRUE | 216 | 2004 |
| 156 | 643 | 600 | 4.055383147379758 | TRUE | 600 | 300 |
| 157 | 916 | 600 | 4.888486296187698 | TRUE | 600 | 300 |
| 158 | 626 | 600 | 2.6601621686112464 | TRUE | 600 | 300 |
| 159 | 746 | 600 | 4.970146668717257 | TRUE | 600 | 300 |
| 160 | 2289 | 216 | 2.561125813271956 | TRUE | 216 | 220 |
| 161 | 1033 | 216 | 1.6732398152133403 | FALSE | 216 | 54 |
| 162 | 781 | 216 | 0.5977781681995396 | TRUE | 216 | 2048 |
| 163 | 998 | 600 | 5 | TRUE | 600 | 300 |
| 164 | 887 | 600 | 5 | TRUE | 600 | 300 |
| 165 | 531 | 600 | 3.862928724584727 | TRUE | 600 | 300 |
| 166 | 604 | 216 | 4.296684744319404 | FALSE | 216 | 114 |
| 167 | 1327 | 216 | 0.8556572072461723 | TRUE | 216 | 1381 |
| 168 | 654 | 216 | 1.3894750079604248 | TRUE | 216 | 109 |
| 169 | 369 | 216 | 2.442170665 | TRUE | 216 | 96 |
| 170 | 947 | 216 | 0.5008597690424667 | TRUE | 216 | 2011 |
| 171 | 923 | 216 | 0.5 | TRUE | 216 | 1056 |
| 172 | 893 | 216 | 0.5388560175380243 | TRUE | 216 | 1725 |
| 173 | 987 | 600 | 5 | TRUE | 600 | 300 |
| 174 | 1128 | 600 | 4.594840157836396 | TRUE | 600 | 300 |
| 175 | 829 | 600 | 5 | TRUE | 600 | 300 |
| 176 | 720 | 600 | 5 | TRUE | 600 | 300 |
| 177 | 978 | 600 | 5 | TRUE | 600 | 300 |
| 178 | 576 | 600 | 4.451172711679753 | FALSE | 600 | 300 |
| 179 | 537 | 600 | 4.148762318423421 | TRUE | 600 | 300 |
| 180 | 1155 | 600 | 4.480109837498329 | TRUE | 600 | 300 |
| 181 | 316 | 600 | 2.557320010855224 | TRUE | 600 | 300 |
| 182 | 193 | 216 | 3.7239830126744193 | TRUE | 216 | 291 |
| 183 | 885 | 600 | 5 | TRUE | 600 | 300 |
| 184 | 759 | 600 | 4.998924363939628 | TRUE | 600 | 300 |
| 185 | 629 | 600 | 4.999557514204478 | TRUE | 600 | 300 |
| 186 | 861 | 600 | 4.998923106801472 | TRUE | 600 | 300 |
| 187 | 223 | 216 | 2.6402467105758936 | TRUE | 216 | 140 |
| 188 | 645 | 600 | 4.949344233740176 | TRUE | 600 | 300 |
| 189 | 789 | 600 | 4.9967476929855446 | TRUE | 600 | 300 |
| 190 | 939 | 216 | 3.337457140281324 | TRUE | 216 | 260 |
| 191 | 1212 | 216 | 0.5129119679235525 | TRUE | 216 | 1887 |
| 192 | 661 | 600 | 5 | TRUE | 600 | 300 |
| 193 | 598 | 600 | 5 | TRUE | 600 | 300 |
| 194 | 130 | 600 | 4.224378713276479 | TRUE | 600 | 300 |
| 195 | 778 | 600 | 5 | TRUE | 600 | 300 |
| 196 | 486 | 600 | 3.8367990348940815 | TRUE | 600 | 300 |
| 197 | 1042 | 600 | 5 | TRUE | 600 | 300 |
| 198 | 822 | 600 | 5 | TRUE | 600 | 300 |
| 199 | 377 | 600 | 4.145111395224619 | TRUE | 600 | 300 |
| 200 | 599 | 600 | 4.871324829 | TRUE | 600 | 300 |
| 201 | 595 | 600 | 5 | TRUE | 600 | 300 |
| 202 | 373 | 216 | 2.334399879 | TRUE | 216 | 194 |
| 203 | 1074 | 600 | 4.086968735 | TRUE | 600 | 300 |
| 204 | 1316 | 216 | 0.5 | TRUE | 216 | 2045 |
| 205 | 744 | 600 | 4.860342274641504 | TRUE | 600 | 300 |
| 206 | 858 | 600 | 5 | TRUE | 600 | 300 |
| 207 | 1540 | 216 | 2.113278368856591 | TRUE | 216 | 61 |
| 208 | 598 | 600 | 3.705215753749509 | TRUE | 600 | 300 |
| 209 | 1074 | 600 | 4.869775473892759 | TRUE | 600 | 300 |
| 210 | 447 | 216 | 3.335557509709549 | TRUE | 216 | 903 |
| 211 | 434 | 600 | 3.254171746995819 | TRUE | 600 | 300 |
| 212 | 1058 | 600 | 4.926645411425534 | TRUE | 600 | 300 |
| 213 | 599 | 216 | 3.1650311742034285 | TRUE | 216 | 179 |
| 214 | 775 | 600 | 5 | TRUE | 600 | 300 |
| 215 | 611 | 600 | 2.529043098915113 | FALSE | 600 | 300 |
| 216 | 549 | 600 | 5 | TRUE | 600 | 300 |
| 217 | 800 | 600 | 4.249114153824346 | TRUE | 600 | 300 |
| 218 | 834 | 600 | 5 | TRUE | 600 | 300 |
| 219 | 812 | 600 | 5 | TRUE | 600 | 300 |
| 220 | 848 | 600 | 4.818705990996169 | TRUE | 600 | 300 |
| 221 | 967 | 600 | 4.99477869 | TRUE | 600 | 300 |
| 222 | 789 | 600 | 5 | TRUE | 600 | 300 |
| 223 | 438 | 600 | 2.7074790200201004 | FALSE | 600 | 300 |
| 224 | 593 | 600 | 4.889864282806045 | TRUE | 600 | 300 |
| 225 | 526 | 216 | 3.2563553283560944 | TRUE | 216 | 155 |
| 226 | 687 | 216 | 3.1293344189459136 | TRUE | 216 | 101 |
| 227 | 1124 | 216 | 2.5358888076124506 | TRUE | 216 | 245 |
| 228 | 1114 | 600 | 4.200002323720642 | TRUE | 600 | 300 |
| 229 | 359 | 216 | 3.376852897907632 | TRUE | 216 | 1652 |
| 230 | 677 | 216 | 2.559958633333142 | TRUE | 216 | 105 |
| 231 | 1533 | 600 | 4.992101205119068 | TRUE | 600 | 300 |
| 232 | 1027 | 600 | 4.146258527288671 | TRUE | 600 | 300 |
| 233 | 761 | 600 | 3.575390889455974 | TRUE | 600 | 300 |
| 234 | 970 | 216 | 0.5 | FALSE | 216 | 2048 |
| 235 | 772 | 600 | 4.989671443285182 | TRUE | 600 | 300 |
| 236 | 1311 | 600 | 4.111849573162523 | FALSE | 600 | 300 |
| 237 | 976 | 600 | 5 | TRUE | 600 | 300 |
| 238 | 841 | 600 | 5 | TRUE | 600 | 300 |
| 239 | 920 | 600 | 5 | TRUE | 600 | 300 |
| 240 | 211 | 600 | 4.408452987741098 | TRUE | 600 | 300 |
| 241 | 554 | 216 | 2.2815813689835287 | TRUE | 216 | 261 |
| 242 | 1199 | 600 | 4.993639234703457 | TRUE | 600 | 300 |
| 243 | 657 | 600 | 4.718676922457268 | TRUE | 600 | 300 |
| 244 | 790 | 216 | 2.7620150068456537 | TRUE | 216 | 259 |
| 245 | 814 | 216 | 2.287422964398144 | TRUE | 216 | 285 |
| 246 | 798 | 600 | 5 | TRUE | 600 | 300 |
| 247 | 770 | 216 | 3.686294029311824 | TRUE | 216 | 123 |
| 248 | 649 | 600 | 4.990630804734721 | TRUE | 600 | 300 |
| 249 | 410 | 216 | 2.8785318037569247 | TRUE | 216 | 767 |
| 250 | 607 | 216 | 0.6784920784274352 | FALSE | 216 | 1002 |
| 251 | 682 | 600 | 5 | TRUE | 600 | 300 |
| 252 | 273 | 216 | 0.663807698 | TRUE | 216 | 1956 |
| 253 | 793 | 600 | 5 | TRUE | 600 | 300 |
| 254 | 1293 | 216 | 0.5 | TRUE | 216 | 1453 |
| 255 | 1267 | 216 | 4.697511587533644 | FALSE | 216 | 76 |
| 256 | 1518 | 600 | 4.981992582743865 | TRUE | 600 | 300 |
| 257 | 1239 | 216 | 1.0470039027066187 | TRUE | 216 | 2048 |
| 258 | 834 | 216 | 0.5 | TRUE | 216 | 1834 |
| 259 | 236 | 600 | 4.191856120155613 | TRUE | 600 | 300 |
| 260 | 818 | 600 | 4.991436448707101 | TRUE | 600 | 300 |
| 261 | 1214 | 216 | 0.5 | TRUE | 216 | 1588 |
| 262 | 806 | 600 | 5 | TRUE | 600 | 300 |
| 263 | 906 | 600 | 5 | TRUE | 600 | 300 |
| 264 | 834 | 216 | 0.5 | TRUE | 216 | 1174 |
| 265 | 728 | 216 | 0.5060899222527042 | TRUE | 216 | 1213 |
| 266 | 1465 | 216 | 0.5047432104487863 | TRUE | 216 | 1802 |
| 267 | 644 | 600 | 4.998722873021777 | TRUE | 600 | 300 |
| 268 | 270 | 600 | 4.999470401565364 | TRUE | 600 | 300 |
| 269 | 563 | 216 | 1.7150001929175602 | TRUE | 216 | 1331 |
| 270 | 178 | 216 | 2.961258437512001 | TRUE | 216 | 399 |
| 271 | 508 | 600 | 2.8159692702335475 | TRUE | 600 | 300 |
| 272 | 995 | 600 | 5 | TRUE | 600 | 300 |
| 273 | 1450 | 216 | 1.1615963720224567 | TRUE | 216 | 2047 |
| 274 | 617 | 600 | 4.997020077538673 | TRUE | 600 | 300 |
| 275 | 559 | 600 | 4.986593315491613 | TRUE | 600 | 300 |
| 276 | 746 | 600 | 4.997446351720846 | TRUE | 600 | 300 |
| 277 | 2008 | 216 | 3.932893868734707 | TRUE | 216 | 185 |
| 278 | 262 | 600 | 4.235723074653879 | TRUE | 600 | 300 |
| 279 | 631 | 216 | 0.5125872577572907 | TRUE | 216 | 868 |
| 280 | 1124 | 600 | 4.756160549154127 | TRUE | 600 | 300 |
| 281 | 912 | 600 | 4.987669455671698 | TRUE | 600 | 300 |
| 282 | 435 | 600 | 4.400228994673315 | TRUE | 600 | 300 |
| 283 | 611 | 600 | 5 | TRUE | 600 | 300 |
| 284 | 744 | 600 | 4.985692541153688 | TRUE | 600 | 300 |
| 285 | 398 | 216 | 3.074686409019592 | FALSE | 216 | 117 |
| 286 | 1053 | 600 | 4.985329468366589 | TRUE | 600 | 300 |
| 287 | 1063 | 600 | 4.908360653698164 | TRUE | 600 | 300 |
| 288 | 569 | 600 | 4.876752689301244 | TRUE | 600 | 300 |
| 289 | 570 | 600 | 4.991705037 | TRUE | 600 | 300 |
| 290 | 862 | 600 | 4.778742384440984 | TRUE | 600 | 300 |
| 291 | 1549 | 216 | 0.5 | TRUE | 216 | 686 |
| 292 | 697 | 600 | 4.999234509222721 | TRUE | 600 | 300 |
| 293 | 834 | 600 | 4.988523765847357 | TRUE | 600 | 300 |
| 294 | 661 | 216 | 1.4469394878471362 | TRUE | 216 | 204 |
| 295 | 633 | 600 | 4.997062669 | TRUE | 600 | 300 |
| 296 | 765 | 600 | 4.994798510691007 | TRUE | 600 | 300 |
| 297 | 779 | 600 | 4.929532550618685 | TRUE | 600 | 300 |
| 298 | 810 | 600 | 5 | TRUE | 600 | 300 |
| 299 | 829 | 600 | 4.999660780521265 | TRUE | 600 | 300 |
| 300 | 721 | 600 | 4.984456165786825 | TRUE | 600 | 300 |
| 301 | 533 | 600 | 4.729833159155193 | TRUE | 600 | 300 |
| 302 | 827 | 600 | 4.924623238326591 | TRUE | 600 | 300 |
| 303 | 827 | 600 | 5 | TRUE | 600 | 300 |
| 304 | 222 | 600 | 2.2534612531316047 | TRUE | 600 | 300 |
| 305 | 240 | 216 | 4.6726732898215335 | TRUE | 216 | 1886 |
| 306 | 457 | 216 | 1.0979605940071182 | TRUE | 216 | 2023 |
| 307 | 356 | 600 | 3.1895225151113453 | TRUE | 600 | 300 |
| 308 | 1006 | 216 | 0.6556962249896598 | FALSE | 216 | 2025 |
| 309 | 761 | 600 | 4.927401530345983 | TRUE | 600 | 300 |
| 310 | 563 | 600 | 4.605826199652198 | TRUE | 600 | 300 |
| 311 | 346 | 600 | 4.688025217585381 | TRUE | 600 | 300 |
| 312 | 185 | 216 | 2.5020558160401585 | TRUE | 216 | 1797 |
| 313 | 714 | 600 | 4.950231109027204 | TRUE | 600 | 300 |
| 314 | 1578 | 216 | 0.5010592290988579 | TRUE | 216 | 2012 |
| 315 | 556 | 600 | 4.898067266553962 | TRUE | 600 | 300 |
| 316 | 905 | 600 | 4.99858466 | TRUE | 600 | 300 |
| 317 | 293 | 216 | 0.5 | FALSE | 216 | 222 |
| 318 | 1045 | 600 | 4.457143938780231 | TRUE | 600 | 300 |
| 319 | 792 | 600 | 4.188751947577948 | TRUE | 600 | 300 |
| 320 | 1042 | 600 | 4.0655739978072685 | TRUE | 600 | 300 |
| 321 | 802 | 600 | 4.983933608970125 | TRUE | 600 | 300 |
| 322 | 520 | 216 | 1.6891330950651278 | TRUE | 216 | 941 |
| 323 | 823 | 600 | 4.994119468536007 | TRUE | 600 | 300 |
| 324 | 692 | 600 | 4.747186448166698 | FALSE | 600 | 300 |
| 325 | 1150 | 600 | 4.9818802572536764 | TRUE | 600 | 300 |
| 326 | 326 | 600 | 2.407190747847167 | TRUE | 600 | 300 |
| 327 | 541 | 600 | 4.9570174621664265 | TRUE | 600 | 300 |
| 328 | 1462 | 216 | 0.5039995816212988 | TRUE | 216 | 1896 |
| 329 | 864 | 600 | 4.802361085147316 | TRUE | 600 | 300 |
| 330 | 839 | 600 | 4.937296026668228 | TRUE | 600 | 300 |
| 331 | 588 | 600 | 2.800742573354102 | TRUE | 600 | 300 |
| 332 | 602 | 600 | 4.970455682 | TRUE | 600 | 300 |
| 333 | 497 | 600 | 2.9767272463976933 | TRUE | 600 | 300 |
| 334 | 757 | 600 | 2.798385390230251 | TRUE | 600 | 300 |
| 335 | 3072 | 216 | 3.555565514860937 | FALSE | 216 | 640 |
| 336 | 562 | 216 | 3.788598434316656 | FALSE | 216 | 471 |
| 337 | 705 | 600 | 4.930708442915843 | TRUE | 600 | 300 |
| 338 | 614 | 600 | 4.935103802231278 | TRUE | 600 | 300 |
| 339 | 128 | 216 | 3.764059951617332 | FALSE | 216 | 851 |
| 340 | 320 | 600 | 3.2953157379938096 | TRUE | 600 | 300 |
| 341 | 301 | 600 | 3.155441291346403 | TRUE | 600 | 300 |
| 342 | 652 | 216 | 0.5 | FALSE | 216 | 1936 |
| 343 | 877 | 600 | 4.795451763138481 | TRUE | 600 | 300 |
| 344 | 1077 | 216 | 0.5390352433961922 | TRUE | 216 | 306 |
| 345 | 1018 | 600 | 4.936587860466565 | TRUE | 600 | 300 |
| 346 | 791 | 600 | 5 | TRUE | 600 | 300 |
| 347 | 909 | 600 | 4.907264163557268 | TRUE | 600 | 300 |
| 348 | 1340 | 216 | 3.219451318534938 | TRUE | 216 | 148 |
| 349 | 568 | 216 | 1.6479536680868605 | TRUE | 216 | 531 |
| 350 | 1035 | 600 | 4.992180109978972 | TRUE | 600 | 300 |
| 351 | 686 | 600 | 5 | TRUE | 600 | 300 |
| 352 | 571 | 600 | 5 | TRUE | 600 | 300 |
| 353 | 307 | 216 | 2.912622640041571 | FALSE | 216 | 299 |
| 354 | 767 | 600 | 4.787262553285323 | TRUE | 600 | 300 |
| 355 | 1717 | 216 | 1.3006766757531762 | TRUE | 216 | 98 |
| 356 | 431 | 216 | 1.804066985948928 | TRUE | 216 | 976 |
| 357 | 327 | 600 | 3.610654392358285 | TRUE | 600 | 300 |
| 358 | 905 | 600 | 5 | TRUE | 600 | 300 |
| 359 | 1159 | 216 | 3.313250328232061 | TRUE | 216 | 79 |
| 360 | 581 | 600 | 4.946343136004145 | TRUE | 600 | 300 |
| 361 | 359 | 216 | 1.7940330043235264 | FALSE | 216 | 251 |
| 362 | 1353 | 600 | 5 | TRUE | 600 | 300 |
| 363 | 961 | 600 | 4.394068862972704 | TRUE | 600 | 300 |
| 364 | 878 | 600 | 5 | TRUE | 600 | 300 |
| 365 | 848 | 600 | 5 | TRUE | 600 | 300 |
| 366 | 812 | 600 | 5 | TRUE | 600 | 300 |
| 367 | 777 | 600 | 5 | TRUE | 600 | 300 |
| 368 | 427 | 216 | 3.834870768177062 | TRUE | 216 | 527 |
| 369 | 916 | 216 | 3.9208199673712856 | TRUE | 216 | 97 |
| 370 | 1038 | 600 | 4.196593575881632 | TRUE | 600 | 300 |
| 371 | 704 | 216 | 2.6866559837429884 | TRUE | 216 | 125 |
| 372 | 842 | 600 | 5 | TRUE | 600 | 300 |
| 373 | 1362 | 216 | 0.5091413549452658 | TRUE | 216 | 1864 |
| 374 | 375 | 600 | 3.639448803404649 | TRUE | 600 | 300 |
| 375 | 1227 | 216 | 0.5278184884414753 | TRUE | 216 | 2048 |
| 376 | 390 | 216 | 3.402599482224166 | TRUE | 216 | 160 |
| 377 | 516 | 216 | 2.048298061594422 | TRUE | 216 | 560 |
| 378 | 880 | 600 | 4.994501974154146 | TRUE | 600 | 300 |
| 379 | 1181 | 216 | 0.502065947 | TRUE | 216 | 621 |
| 380 | 1515 | 216 | 5 | TRUE | 216 | 35 |
| 381 | 1177 | 600 | 5 | TRUE | 600 | 300 |
| 382 | 316 | 600 | 3.1161049753149017 | TRUE | 600 | 300 |
| 383 | 1010 | 600 | 4.775110554002692 | TRUE | 600 | 300 |
| 384 | 960 | 600 | 3.8770612463517584 | TRUE | 600 | 300 |
| 385 | 864 | 600 | 5 | TRUE | 600 | 300 |
| 386 | 462 | 600 | 3.6723718869666166 | TRUE | 600 | 300 |
| 387 | 786 | 600 | 4.900295225714911 | TRUE | 600 | 300 |
| 388 | 1622 | 216 | 3.123314102558143 | TRUE | 216 | 135 |
| 389 | 581 | 216 | 3.9139214783122656 | TRUE | 216 | 105 |
| 390 | 189 | 216 | 3.1041932671596184 | TRUE | 216 | 219 |
| 391 | 2909 | 600 | 0.8111034500763327 | FALSE | 600 | 300 |
| 392 | 549 | 600 | 4.787603370621028 | TRUE | 600 | 300 |
| 393 | 679 | 600 | 4.926593422374405 | TRUE | 600 | 300 |
| 394 | 556 | 600 | 4.948176976138139 | TRUE | 600 | 300 |
| 395 | 686 | 600 | 4.998082606578591 | TRUE | 600 | 300 |
| 396 | 439 | 216 | 1.9037091029377824 | FALSE | 216 | 121 |
| 397 | 963 | 600 | 4.9986118606403735 | TRUE | 600 | 300 |
| 398 | 519 | 216 | 1.3160050099684435 | TRUE | 216 | 183 |
| 399 | 680 | 600 | 2.6837060484966613 | TRUE | 600 | 300 |
| 400 | 965 | 600 | 5 | TRUE | 600 | 300 |
| 401 | 1026 | 600 | 5 | TRUE | 600 | 300 |
| 402 | 355 | 600 | 4.702991920153124 | FALSE | 600 | 300 |
| 403 | 440 | 600 | 3.329288090023353 | TRUE | 600 | 300 |
| 404 | 327 | 600 | 3.0386135120137525 | TRUE | 600 | 300 |
| 405 | 530 | 600 | 3.279656831253829 | TRUE | 600 | 300 |
| 406 | 654 | 600 | 4.425899351396507 | TRUE | 600 | 300 |
| 407 | 867 | 216 | 0.5 | TRUE | 216 | 1926 |
| 408 | 1112 | 600 | 4.168403484418555 | TRUE | 600 | 300 |
| 409 | 695 | 600 | 5 | TRUE | 600 | 300 |
| 410 | 901 | 600 | 4.776607034319936 | TRUE | 600 | 300 |
| 411 | 1493 | 216 | 2.8600162206478768 | TRUE | 216 | 140 |
| 412 | 775 | 216 | 3.061847456414164 | FALSE | 216 | 120 |
| 413 | 338 | 216 | 2.206918201421881 | TRUE | 216 | 429 |
| 414 | 835 | 600 | 4.999599645881936 | TRUE | 600 | 300 |
| 415 | 1396 | 600 | 3.141711684079307 | TRUE | 600 | 300 |
| 416 | 647 | 600 | 4.9483110415573694 | TRUE | 600 | 300 |
| 417 | 817 | 600 | 5 | TRUE | 600 | 300 |
| 418 | 586 | 600 | 4.963052745552937 | TRUE | 600 | 300 |
| 419 | 1097 | 600 | 4.892320645989617 | TRUE | 600 | 300 |
| 420 | 771 | 600 | 4.976339310455634 | TRUE | 600 | 300 |
| 421 | 717 | 600 | 5 | TRUE | 600 | 300 |
| 422 | 850 | 600 | 4.940640253521899 | TRUE | 600 | 300 |
| 423 | 802 | 600 | 4.972560471140315 | TRUE | 600 | 300 |
| 424 | 946 | 600 | 4.895742459196234 | TRUE | 600 | 300 |
| 425 | 1244 | 600 | 2.664732909212218 | TRUE | 600 | 300 |
| 426 | 715 | 600 | 4.998736060521181 | TRUE | 600 | 300 |
| 427 | 933 | 600 | 5 | TRUE | 600 | 300 |
| 428 | 778 | 216 | 4.495084725 | TRUE | 216 | 346 |
| 429 | 878 | 600 | 4.964016209752554 | TRUE | 600 | 300 |
| 430 | 388 | 600 | 3.3668798582448005 | TRUE | 600 | 300 |
| 431 | 850 | 600 | 4.695253038144988 | TRUE | 600 | 300 |
| 432 | 790 | 216 | 0.6533014834544647 | TRUE | 216 | 544 |
| 433 | 809 | 600 | 5 | TRUE | 600 | 300 |
| 434 | 128 | 216 | 2.0499078277951908 | TRUE | 216 | 70 |
| 435 | 877 | 216 | 0.6675189670152127 | TRUE | 216 | 80 |
| 436 | 891 | 600 | 4.927155464072142 | TRUE | 600 | 300 |
| 437 | 921 | 600 | 5 | TRUE | 600 | 300 |
| 438 | 818 | 600 | 4.999878830318461 | TRUE | 600 | 300 |
| 439 | 1632 | 216 | 0.509672094 | TRUE | 216 | 200 |
| 440 | 793 | 600 | 5 | TRUE | 600 | 300 |
| 441 | 1104 | 600 | 5 | TRUE | 600 | 300 |
| 442 | 1076 | 600 | 4.337359767252176 | TRUE | 600 | 300 |
| 443 | 869 | 600 | 5 | TRUE | 600 | 300 |
| 444 | 1538 | 216 | 0.5167081507425422 | TRUE | 216 | 2048 |
| 445 | 825 | 600 | 4.988432713443188 | TRUE | 600 | 300 |
| 446 | 573 | 600 | 4.902568381279489 | TRUE | 600 | 300 |
| 447 | 841 | 600 | 4.999644645 | TRUE | 600 | 300 |
| 448 | 731 | 600 | 5 | TRUE | 600 | 300 |
| 449 | 884 | 600 | 4.883397307436822 | TRUE | 600 | 300 |
| 450 | 734 | 600 | 5 | TRUE | 600 | 300 |
| 451 | 801 | 216 | 1.217926274143726 | TRUE | 216 | 1899 |
| 452 | 804 | 600 | 4.957624690984805 | TRUE | 600 | 300 |
| 453 | 136 | 600 | 3.1865925804881803 | TRUE | 600 | 300 |
| 454 | 3072 | 600 | 5 | TRUE | 600 | 300 |
| 455 | 678 | 600 | 4.998071067576215 | TRUE | 600 | 300 |
| 456 | 853 | 600 | 4.981919978 | TRUE | 600 | 300 |
| 457 | 1022 | 600 | 2.303156378384101 | FALSE | 600 | 300 |
| 458 | 1094 | 600 | 4.9604038215980895 | TRUE | 600 | 300 |
| 459 | 911 | 600 | 4.965585403839819 | TRUE | 600 | 300 |
| 460 | 975 | 216 | 1.0029949117549397 | TRUE | 216 | 511 |
| 461 | 843 | 600 | 5 | TRUE | 600 | 300 |
| 462 | 823 | 600 | 5 | TRUE | 600 | 300 |
| 463 | 463 | 600 | 3.0390427184967352 | TRUE | 600 | 300 |
| 464 | 663 | 600 | 4.310014811521936 | TRUE | 600 | 300 |
| 465 | 670 | 600 | 4.907122704493322 | TRUE | 600 | 300 |
| 466 | 283 | 600 | 3.494455639356576 | TRUE | 600 | 300 |
| 467 | 1455 | 600 | 4.595442697080213 | TRUE | 600 | 300 |
| 468 | 744 | 600 | 5 | TRUE | 600 | 300 |
| 469 | 137 | 600 | 3.111051932616059 | TRUE | 600 | 300 |
| 470 | 163 | 600 | 4.147231132358007 | TRUE | 600 | 300 |
| 471 | 1303 | 600 | 4.1090182078734365 | TRUE | 600 | 300 |
| 472 | 545 | 600 | 4.293371423383739 | TRUE | 600 | 300 |
| 473 | 530 | 600 | 4.905012336159218 | TRUE | 600 | 300 |
| 474 | 808 | 600 | 4.998874772391473 | TRUE | 600 | 300 |
| 475 | 1100 | 600 | 4.223220668 | TRUE | 600 | 300 |
| 476 | 586 | 600 | 2.7593427045331977 | TRUE | 600 | 300 |
| 477 | 395 | 600 | 5 | TRUE | 600 | 300 |
| 478 | 597 | 600 | 4.650208833814583 | TRUE | 600 | 300 |
| 479 | 527 | 600 | 3.2409195273351097 | FALSE | 600 | 300 |
| 480 | 522 | 600 | 4.334004074745083 | TRUE | 600 | 300 |
| 481 | 1472 | 216 | 3.5584722002724063 | TRUE | 216 | 1709 |
| 482 | 1488 | 216 | 0.6363928734832068 | TRUE | 216 | 1999 |
| 483 | 1534 | 600 | 4.859327317624337 | FALSE | 600 | 300 |
| 484 | 1456 | 216 | 0.5185050757489857 | TRUE | 216 | 2048 |
| 485 | 582 | 600 | 4.900441028 | TRUE | 600 | 300 |
| 486 | 555 | 600 | 3.903187187914503 | TRUE | 600 | 300 |
| 487 | 870 | 216 | 1.123961413242951 | TRUE | 216 | 423 |
| 488 | 1408 | 216 | 0.5116464287465591 | FALSE | 216 | 2036 |
| 489 | 646 | 600 | 4.510651476411479 | TRUE | 600 | 300 |
| 490 | 779 | 600 | 3.6304416944747127 | TRUE | 600 | 300 |
| 491 | 644 | 600 | 4.995813681630708 | TRUE | 600 | 300 |
| 492 | 652 | 600 | 4.727044230250208 | TRUE | 600 | 300 |

| Row ID | conv1_kernel_size | conv2_channels | conv2_kernel_size | conv3_channels | conv3_kernel_size | n_linear_layers |
|---|---|---|---|---|---|---|
| 1 | 19 | 200 | 11 | 200 | 7 | 2 |
| 2 | 19 | 200 | 11 | 200 | 7 | 1 |
| 3 | 13 | 2030 | 5 | 16 | 25 | 2 |
| 4 | 19 | 200 | 11 | 200 | 7 | 3 |
| 5 | 19 | 200 | 11 | 200 | 7 | 1 |
| 6 | 19 | 200 | 11 | 200 | 7 | 2 |
| 7 | 10 | 58 | 13 | 59 | 22 | 2 |
| 8 | 19 | 200 | 11 | 200 | 7 | 3 |
| 9 | 6 | 182 | 9 | 26 | 14 | 2 |
| 10 | 19 | 200 | 11 | 200 | 7 | 1 |
| 11 | 19 | 200 | 11 | 200 | 7 | 3 |
| 12 | 19 | 200 | 11 | 200 | 7 | 3 |
| 13 | 19 | 200 | 11 | 200 | 7 | 2 |
| 14 | 7 | 72 | 12 | 30 | 15 | 2 |
| 15 | 19 | 200 | 11 | 200 | 7 | 2 |
| 16 | 19 | 200 | 11 | 200 | 7 | 2 |
| 17 | 19 | 200 | 11 | 200 | 7 | 1 |
| 18 | 19 | 200 | 11 | 200 | 7 | 3 |
| 19 | 19 | 200 | 11 | 200 | 7 | 4 |
| 20 | 19 | 200 | 11 | 200 | 7 | 1 |
| 21 | 19 | 200 | 11 | 200 | 7 | 3 |
| 22 | 19 | 200 | 11 | 200 | 7 | 1 |
| 23 | 16 | 199 | 12 | 21 | 14 | 2 |
| 24 | 19 | 200 | 11 | 200 | 7 | 1 |
| 25 | 19 | 200 | 11 | 200 | 7 | 1 |
| 26 | 19 | 200 | 11 | 200 | 7 | 1 |
| 27 | 19 | 200 | 11 | 200 | 7 | 1 |
| 28 | 19 | 200 | 11 | 200 | 7 | 2 |
| 29 | 19 | 200 | 11 | 200 | 7 | 2 |
| 30 | 19 | 200 | 11 | 200 | 7 | 3 |
| 31 | 19 | 200 | 11 | 200 | 7 | 1 |
| 32 | 19 | 200 | 11 | 200 | 7 | 1 |
| 33 | 17 | 136 | 24 | 377 | 15 | 3 |
| 34 | 19 | 200 | 11 | 200 | 7 | 2 |
| 35 | 19 | 200 | 11 | 200 | 7 | 1 |
| 36 | 6 | 33 | 6 | 117 | 5 | 1 |
| 37 | 19 | 200 | 11 | 200 | 7 | 3 |
| 38 | 16 | 1968 | 6 | 16 | 25 | 1 |
| 39 | 19 | 200 | 11 | 200 | 7 | 1 |
| 40 | 19 | 200 | 11 | 200 | 7 | 3 |
| 41 | 14 | 244 | 5 | 21 | 17 | 2 |
| 42 | 19 | 200 | 11 | 200 | 7 | 1 |
| 43 | 25 | 118 | 8 | 1672 | 25 | 3 |
| 44 | 19 | 200 | 11 | 200 | 7 | 3 |
| 45 | 13 | 86 | 15 | 88 | 12 | 2 |
| 46 | 18 | 205 | 10 | 53 | 10 | 3 |
| 47 | 19 | 200 | 11 | 200 | 7 | 1 |
| 48 | 19 | 200 | 11 | 200 | 7 | 2 |
| 49 | 19 | 200 | 11 | 200 | 7 | 1 |
| 50 | 19 | 200 | 11 | 200 | 7 | 2 |
| 51 | 8 | 1484 | 5 | 28 | 19 | 2 |
| 52 | 19 | 200 | 11 | 200 | 7 | 3 |
| 53 | 19 | 200 | 11 | 200 | 7 | 1 |
| 54 | 19 | 200 | 11 | 200 | 7 | 1 |
| 55 | 19 | 200 | 11 | 200 | 7 | 2 |
| 56 | 13 | 206 | 16 | 56 | 20 | 2 |
| 57 | 19 | 200 | 11 | 200 | 7 | 1 |
| 58 | 19 | 200 | 11 | 200 | 7 | 3 |
| 59 | 19 | 200 | 11 | 200 | 7 | 3 |
| 60 | 19 | 200 | 11 | 200 | 7 | 3 |
| 61 | 19 | 200 | 11 | 200 | 7 | 1 |
| 62 | 19 | 200 | 11 | 200 | 7 | 4 |
| 63 | 19 | 200 | 11 | 200 | 7 | 2 |
| 64 | 19 | 200 | 11 | 200 | 7 | 4 |
| 65 | 19 | 200 | 11 | 200 | 7 | 1 |
| 66 | 19 | 200 | 11 | 200 | 7 | 2 |
| 67 | 19 | 200 | 11 | 200 | 7 | 3 |
| 68 | 19 | 200 | 11 | 200 | 7 | 1 |
| 69 | 19 | 200 | 11 | 200 | 7 | 2 |
| 70 | 19 | 200 | 11 | 200 | 7 | 1 |
| 71 | 19 | 200 | 11 | 200 | 7 | 2 |
| 72 | 6 | 574 | 11 | 16 | 24 | 3 |
| 73 | 5 | 37 | 12 | 98 | 11 | 1 |
| 74 | 24 | 52 | 13 | 527 | 22 | 4 |
| 75 | 19 | 200 | 11 | 200 | 7 | 4 |
| 76 | 19 | 200 | 11 | 200 | 7 | 1 |
| 77 | 6 | 175 | 23 | 49 | 21 | 4 |
| 78 | 19 | 200 | 11 | 200 | 7 | 3 |
| 79 | 5 | 742 | 9 | 16 | 22 | 2 |
| 80 | 19 | 200 | 11 | 200 | 7 | 3 |
| 81 | 19 | 200 | 11 | 200 | 7 | 2 |
| 82 | 19 | 200 | 11 | 200 | 7 | 3 |
| 83 | 19 | 200 | 11 | 200 | 7 | 3 |
| 84 | 17 | 434 | 10 | 19 | 21 | 2 |
| 85 | 19 | 200 | 11 | 200 | 7 | 2 |
| 86 | 19 | 200 | 11 | 200 | 7 | 2 |
| 87 | 19 | 200 | 11 | 200 | 7 | 5 |
| 88 | 19 | 200 | 11 | 200 | 7 | 1 |
| 89 | 19 | 200 | 11 | 200 | 7 | 1 |
| 90 | 19 | 200 | 11 | 200 | 7 | 3 |
| 91 | 13 | 95 | 15 | 72 | 18 | 2 |
| 92 | 19 | 200 | 11 | 200 | 7 | 1 |
| 93 | 19 | 200 | 11 | 200 | 7 | 1 |
| 94 | 19 | 200 | 11 | 200 | 7 | 3 |
| 95 | 19 | 200 | 11 | 200 | 7 | 3 |
| 96 | 11 | 308 | 12 | 23 | 21 | 2 |
| 97 | 19 | 200 | 11 | 200 | 7 | 3 |
| 98 | 19 | 200 | 11 | 200 | 7 | 1 |
| 99 | 19 | 200 | 11 | 200 | 7 | 1 |
| 100 | 19 | 200 | 11 | 200 | 7 | 2 |
| 101 | 19 | 200 | 11 | 200 | 7 | 1 |
| 102 | 19 | 200 | 11 | 200 | 7 | 2 |
| 103 | 19 | 200 | 11 | 200 | 7 | 2 |
| 104 | 19 | 200 | 11 | 200 | 7 | 1 |
| 105 | 19 | 200 | 11 | 200 | 7 | 1 |
| 106 | 19 | 200 | 11 | 200 | 7 | 4 |
| 107 | 19 | 200 | 11 | 200 | 7 | 1 |
| 108 | 19 | 200 | 11 | 200 | 7 | 1 |
| 109 | 19 | 200 | 11 | 200 | 7 | 2 |
| 110 | 19 | 200 | 11 | 200 | 7 | 2 |
| 111 | 19 | 200 | 11 | 200 | 7 | 4 |
| 112 | 19 | 200 | 11 | 200 | 7 | 1 |
| 113 | 19 | 200 | 11 | 200 | 7 | 3 |
| 114 | 19 | 200 | 11 | 200 | 7 | 4 |
| 115 | 19 | 200 | 11 | 200 | 7 | 1 |
| 116 | 19 | 200 | 11 | 200 | 7 | 1 |
| 117 | 19 | 200 | 11 | 200 | 7 | 2 |
| 118 | 19 | 200 | 11 | 200 | 7 | 1 |
| 119 | 19 | 200 | 11 | 200 | 7 | 3 |
| 120 | 19 | 200 | 11 | 200 | 7 | 3 |
| 121 | 19 | 200 | 11 | 200 | 7 | 1 |
| 122 | 19 | 200 | 11 | 200 | 7 | 2 |
| 123 | 19 | 200 | 11 | 200 | 7 | 3 |
| 124 | 19 | 200 | 11 | 200 | 7 | 3 |
| 125 | 19 | 200 | 11 | 200 | 7 | 1 |
| 126 | 19 | 200 | 11 | 200 | 7 | 1 |
| 127 | 19 | 200 | 11 | 200 | 7 | 1 |
| 128 | 19 | 200 | 11 | 200 | 7 | 2 |
| 129 | 19 | 200 | 11 | 200 | 7 | 2 |
| 130 | 19 | 200 | 11 | 200 | 7 | 4 |
| 131 | 19 | 200 | 11 | 200 | 7 | 3 |
| 132 | 19 | 200 | 11 | 200 | 7 | 2 |
| 133 | 19 | 200 | 11 | 200 | 7 | 1 |
| 134 | 19 | 200 | 11 | 200 | 7 | 3 |
| 135 | 12 | 846 | 16 | 42 | 18 | 2 |
| 136 | 19 | 200 | 11 | 200 | 7 | 2 |
| 137 | 7 | 497 | 13 | 113 | 13 | 2 |
| 138 | 23 | 124 | 6 | 1660 | 24 | 2 |
| 139 | 24 | 1020 | 11 | 682 | 21 | 1 |
| 140 | 14 | 893 | 17 | 16 | 24 | 1 |
| 141 | 19 | 200 | 11 | 200 | 7 | 1 |
| 142 | 19 | 200 | 11 | 200 | 7 | 2 |
| 143 | 11 | 1632 | 11 | 41 | 20 | 3 |
| 144 | 19 | 200 | 11 | 200 | 7 | 1 |
| 145 | 19 | 200 | 11 | 200 | 7 | 1 |
| 146 | 6 | 65 | 17 | 36 | 21 | 3 |
| 147 | 19 | 200 | 11 | 200 | 7 | 2 |
| 148 | 17 | 130 | 6 | 433 | 21 | 2 |
| 149 | 19 | 200 | 11 | 200 | 7 | 4 |
| 150 | 19 | 200 | 11 | 200 | 7 | 2 |
| 151 | 19 | 200 | 11 | 200 | 7 | 2 |
| 152 | 22 | 934 | 7 | 17 | 24 | 1 |
| 153 | 19 | 200 | 11 | 200 | 7 | 2 |
| 154 | 8 | 2048 | 5 | 16 | 15 | 1 |
| 155 | 19 | 2048 | 6 | 17 | 24 | 2 |
| 156 | 19 | 200 | 11 | 200 | 7 | 5 |
| 157 | 19 | 200 | 11 | 200 | 7 | 1 |
| 158 | 19 | 200 | 11 | 200 | 7 | 2 |
| 159 | 19 | 200 | 11 | 200 | 7 | 2 |
| 160 | 6 | 243 | 11 | 38 | 12 | 2 |
| 161 | 16 | 1480 | 18 | 16 | 24 | 2 |
| 162 | 6 | 794 | 21 | 187 | 25 | 3 |
| 163 | 19 | 200 | 11 | 200 | 7 | 1 |
| 164 | 19 | 200 | 11 | 200 | 7 | 1 |
| 165 | 19 | 200 | 11 | 200 | 7 | 1 |
| 166 | 16 | 97 | 21 | 107 | 11 | 2 |
| 167 | 15 | 254 | 13 | 98 | 25 | 1 |
| 168 | 14 | 773 | 21 | 25 | 22 | 3 |
| 169 | 17 | 58 | 17 | 41 | 9 | 1 |
| 170 | 16 | 149 | 6 | 315 | 23 | 2 |
| 171 | 24 | 223 | 6 | 717 | 24 | 2 |
| 172 | 18 | 190 | 7 | 500 | 22 | 1 |
| 173 | 19 | 200 | 11 | 200 | 7 | 3 |
| 174 | 19 | 200 | 11 | 200 | 7 | 2 |
| 175 | 19 | 200 | 11 | 200 | 7 | 1 |
| 176 | 19 | 200 | 11 | 200 | 7 | 3 |
| 177 | 19 | 200 | 11 | 200 | 7 | 1 |
| 178 | 19 | 200 | 11 | 200 | 7 | 1 |
| 179 | 19 | 200 | 11 | 200 | 7 | 1 |
| 180 | 19 | 200 | 11 | 200 | 7 | 2 |
| 181 | 19 | 200 | 11 | 200 | 7 | 2 |
| 182 | 12 | 20 | 24 | 46 | 12 | 4 |
| 183 | 19 | 200 | 11 | 200 | 7 | 4 |
| 184 | 19 | 200 | 11 | 200 | 7 | 2 |
| 185 | 19 | 200 | 11 | 200 | 7 | 1 |
| 186 | 19 | 200 | 11 | 200 | 7 | 1 |
| 187 | 24 | 87 | 23 | 1224 | 12 | 5 |
| 188 | 19 | 200 | 11 | 200 | 7 | 1 |
| 189 | 19 | 200 | 11 | 200 | 7 | 1 |
| 190 | 8 | 95 | 12 | 75 | 11 | 1 |
| 191 | 9 | 2048 | 5 | 41 | 24 | 1 |
| 192 | 19 | 200 | 11 | 200 | 7 | 3 |
| 193 | 19 | 200 | 11 | 200 | 7 | 3 |
| 194 | 19 | 200 | 11 | 200 | 7 | 2 |
| 195 | 19 | 200 | 11 | 200 | 7 | 1 |
| 196 | 19 | 200 | 11 | 200 | 7 | 3 |
| 197 | 19 | 200 | 11 | 200 | 7 | 1 |
| 198 | 19 | 200 | 11 | 200 | 7 | 3 |
| 199 | 19 | 200 | 11 | 200 | 7 | 2 |
| 200 | 19 | 200 | 11 | 200 | 7 | 2 |
| 201 | 19 | 200 | 11 | 200 | 7 | 2 |
| 202 | 19 | 140 | 16 | 45 | 11 | 1 |
| 203 | 19 | 200 | 11 | 200 | 7 | 4 |
| 204 | 15 | 2048 | 6 | 16 | 22 | 1 |
| 205 | 19 | 200 | 11 | 200 | 7 | 2 |
| 206 | 19 | 200 | 11 | 200 | 7 | 1 |
| 207 | 18 | 30 | 19 | 93 | 12 | 1 |
| 208 | 19 | 200 | 11 | 200 | 7 | 2 |
| 209 | 19 | 200 | 11 | 200 | 7 | 1 |
| 210 | 18 | 734 | 21 | 36 | 24 | 2 |
| 211 | 19 | 200 | 11 | 200 | 7 | 3 |
| 212 | 19 | 200 | 11 | 200 | 7 | 3 |
| 213 | 9 | 44 | 16 | 60 | 12 | 3 |
| 214 | 19 | 200 | 11 | 200 | 7 | 1 |
| 215 | 19 | 200 | 11 | 200 | 7 | 4 |
| 216 | 19 | 200 | 11 | 200 | 7 | 4 |
| 217 | 19 | 200 | 11 | 200 | 7 | 2 |
| 218 | 19 | 200 | 11 | 200 | 7 | 1 |
| 219 | 19 | 200 | 11 | 200 | 7 | 1 |
| 220 | 19 | 200 | 11 | 200 | 7 | 1 |
| 221 | 19 | 200 | 11 | 200 | 7 | 3 |
| 222 | 19 | 200 | 11 | 200 | 7 | 1 |
| 223 | 19 | 200 | 11 | 200 | 7 | 1 |
| 224 | 19 | 200 | 11 | 200 | 7 | 1 |
| 225 | 14 | 674 | 16 | 227 | 17 | 1 |
| 226 | 14 | 69 | 16 | 50 | 14 | 3 |
| 227 | 11 | 1247 | 21 | 31 | 21 | 1 |
| 228 | 19 | 200 | 11 | 200 | 7 | 2 |
| 229 | 9 | 2048 | 16 | 16 | 24 | 1 |
| 230 | 16 | 157 | 15 | 265 | 10 | 2 |
| 231 | 19 | 200 | 11 | 200 | 7 | 3 |
| 232 | 19 | 200 | 11 | 200 | 7 | 2 |
| 233 | 19 | 200 | 11 | 200 | 7 | 2 |
| 234 | 18 | 562 | 11 | 24 | 25 | 2 |
| 235 | 19 | 200 | 11 | 200 | 7 | 3 |
| 236 | 19 | 200 | 11 | 200 | 7 | 2 |
| 237 | 19 | 200 | 11 | 200 | 7 | 1 |
| 238 | 19 | 200 | 11 | 200 | 7 | 1 |
| 239 | 19 | 200 | 11 | 200 | 7 | 4 |
| 240 | 19 | 200 | 11 | 200 | 7 | 1 |
| 241 | 11 | 133 | 16 | 88 | 14 | 2 |
| 242 | 19 | 200 | 11 | 200 | 7 | 2 |
| 243 | 19 | 200 | 11 | 200 | 7 | 1 |
| 244 | 11 | 2048 | 17 | 16 | 25 | 2 |
| 245 | 19 | 117 | 25 | 409 | 18 | 3 |
| 246 | 19 | 200 | 11 | 200 | 7 | 3 |
| 247 | 20 | 51 | 24 | 1004 | 16 | 4 |
| 248 | 19 | 200 | 11 | 200 | 7 | 3 |
| 249 | 5 | 57 | 15 | 47 | 12 | 2 |
| 250 | 7 | 156 | 12 | 149 | 25 | 3 |
| 251 | 19 | 200 | 11 | 200 | 7 | 1 |
| 252 | 5 | 460 | 18 | 98 | 25 | 3 |
| 253 | 19 | 200 | 11 | 200 | 7 | 1 |
| 254 | 10 | 44 | 5 | 17 | 10 | 2 |
| 255 | 10 | 29 | 15 | 60 | 5 | 2 |
| 256 | 19 | 200 | 11 | 200 | 7 | 3 |
| 257 | 21 | 62 | 5 | 1947 | 25 | 3 |
| 258 | 13 | 2048 | 6 | 16 | 24 | 1 |
| 259 | 19 | 200 | 11 | 200 | 7 | 3 |
| 260 | 19 | 200 | 11 | 200 | 7 | 1 |
| 261 | 12 | 1991 | 5 | 16 | 17 | 1 |
| 262 | 19 | 200 | 11 | 200 | 7 | 1 |
| 263 | 19 | 200 | 11 | 200 | 7 | 3 |
| 264 | 21 | 148 | 8 | 359 | 25 | 3 |
| 265 | 20 | 324 | 9 | 445 | 24 | 2 |
| 266 | 22 | 1440 | 5 | 17 | 21 | 2 |
| 267 | 19 | 200 | 11 | 200 | 7 | 1 |
| 268 | 19 | 200 | 11 | 200 | 7 | 1 |
| 269 | 6 | 290 | 10 | 27 | 25 | 1 |
| 270 | 5 | 212 | 18 | 89 | 20 | 3 |
| 271 | 19 | 200 | 11 | 200 | 7 | 1 |
| 272 | 19 | 200 | 11 | 200 | 7 | 2 |
| 273 | 9 | 912 | 6 | 16 | 21 | 1 |
| 274 | 19 | 200 | 11 | 200 | 7 | 1 |
| 275 | 19 | 200 | 11 | 200 | 7 | 1 |
| 276 | 19 | 200 | 11 | 200 | 7 | 1 |
| 277 | 5 | 153 | 8 | 24 | 12 | 2 |
| 278 | 19 | 200 | 11 | 200 | 7 | 3 |
| 279 | 14 | 156 | 6 | 839 | 20 | 2 |
| 280 | 19 | 200 | 11 | 200 | 7 | 1 |
| 281 | 19 | 200 | 11 | 200 | 7 | 2 |
| 282 | 19 | 200 | 11 | 200 | 7 | 1 |
| 283 | 19 | 200 | 11 | 200 | 7 | 1 |
| 284 | 19 | 200 | 11 | 200 | 7 | 3 |
| 285 | 19 | 147 | 9 | 63 | 13 | 2 |
| 286 | 19 | 200 | 11 | 200 | 7 | 2 |
| 287 | 19 | 200 | 11 | 200 | 7 | 2 |
| 288 | 19 | 200 | 11 | 200 | 7 | 3 |
| 289 | 19 | 200 | 11 | 200 | 7 | 2 |
| 290 | 19 | 200 | 11 | 200 | 7 | 1 |
| 291 | 8 | 193 | 10 | 16 | 17 | 2 |
| 292 | 19 | 200 | 11 | 200 | 7 | 2 |
| 293 | 19 | 200 | 11 | 200 | 7 | 4 |
| 294 | 19 | 540 | 11 | 46 | 8 | 2 |
| 295 | 19 | 200 | 11 | 200 | 7 | 2 |
| 296 | 19 | 200 | 11 | 200 | 7 | 1 |
| 297 | 19 | 200 | 11 | 200 | 7 | 2 |
| 298 | 19 | 200 | 11 | 200 | 7 | 1 |
| 299 | 19 | 200 | 11 | 200 | 7 | 1 |
| 300 | 19 | 200 | 11 | 200 | 7 | 3 |
| 301 | 19 | 200 | 11 | 200 | 7 | 2 |
| 302 | 19 | 200 | 11 | 200 | 7 | 1 |
| 303 | 19 | 200 | 11 | 200 | 7 | 1 |
| 304 | 19 | 200 | 11 | 200 | 7 | 1 |
| 305 | 5 | 1484 | 13 | 19 | 24 | 1 |
| 306 | 24 | 1885 | 6 | 46 | 25 | 2 |
| 307 | 19 | 200 | 11 | 200 | 7 | 1 |
| 308 | 11 | 284 | 12 | 16 | 24 | 2 |
| 309 | 19 | 200 | 11 | 200 | 7 | 1 |
| 310 | 19 | 200 | 11 | 200 | 7 | 2 |
| 311 | 19 | 200 | 11 | 200 | 7 | 3 |
| 312 | 6 | 16 | 16 | 29 | 12 | 2 |
| 313 | 19 | 200 | 11 | 200 | 7 | 2 |
| 314 | 21 | 2048 | 5 | 85 | 25 | 1 |
| 315 | 19 | 200 | 11 | 200 | 7 | 1 |
| 316 | 19 | 200 | 11 | 200 | 7 | 1 |
| 317 | 5 | 25 | 17 | 16 | 25 | 2 |
| 318 | 19 | 200 | 11 | 200 | 7 | 3 |
| 319 | 19 | 200 | 11 | 200 | 7 | 3 |
| 320 | 19 | 200 | 11 | 200 | 7 | 4 |
| 321 | 19 | 200 | 11 | 200 | 7 | 4 |
| 322 | 13 | 104 | 5 | 101 | 19 | 2 |
| 323 | 19 | 200 | 11 | 200 | 7 | 1 |
| 324 | 19 | 200 | 11 | 200 | 7 | 1 |
| 325 | 19 | 200 | 11 | 200 | 7 | 3 |
| 326 | 19 | 200 | 11 | 200 | 7 | 4 |
| 327 | 19 | 200 | 11 | 200 | 7 | 2 |
| 328 | 14 | 1394 | 5 | 29 | 20 | 1 |
| 329 | 19 | 200 | 11 | 200 | 7 | 3 |
| 330 | 19 | 200 | 11 | 200 | 7 | 2 |
| 331 | 19 | 200 | 11 | 200 | 7 | 2 |
| 332 | 19 | 200 | 11 | 200 | 7 | 3 |
| 333 | 19 | 200 | 11 | 200 | 7 | 3 |
| 334 | 19 | 200 | 11 | 200 | 7 | 2 |
| 335 | 6 | 75 | 6 | 93 | 16 | 2 |
| 336 | 25 | 1973 | 24 | 150 | 24 | 1 |
| 337 | 19 | 200 | 11 | 200 | 7 | 2 |
| 338 | 19 | 200 | 11 | 200 | 7 | 4 |
| 339 | 8 | 16 | 24 | 16 | 11 | 4 |
| 340 | 19 | 200 | 11 | 200 | 7 | 1 |
| 341 | 19 | 200 | 11 | 200 | 7 | 1 |
| 342 | 5 | 2048 | 25 | 704 | 25 | 4 |
| 343 | 19 | 200 | 11 | 200 | 7 | 2 |
| 344 | 9 | 2048 | 5 | 23 | 19 | 1 |
| 345 | 19 | 200 | 11 | 200 | 7 | 2 |
| 346 | 19 | 200 | 11 | 200 | 7 | 1 |
| 347 | 19 | 200 | 11 | 200 | 7 | 1 |
| 348 | 11 | 526 | 15 | 44 | 17 | 2 |
| 349 | 11 | 169 | 15 | 60 | 24 | 2 |
| 350 | 19 | 200 | 11 | 200 | 7 | 2 |
| 351 | 19 | 200 | 11 | 200 | 7 | 1 |
| 352 | 19 | 200 | 11 | 200 | 7 | 1 |
| 353 | 22 | 108 | 20 | 649 | 15 | 4 |
| 354 | 19 | 200 | 11 | 200 | 7 | 3 |
| 355 | 12 | 386 | 19 | 62 | 24 | 2 |
| 356 | 15 | 429 | 15 | 63 | 17 | 1 |
| 357 | 19 | 200 | 11 | 200 | 7 | 4 |
| 358 | 19 | 200 | 11 | 200 | 7 | 4 |
| 359 | 5 | 16 | 12 | 31 | 9 | 1 |
| 360 | 19 | 200 | 11 | 200 | 7 | 1 |
| 361 | 9 | 2048 | 14 | 16 | 24 | 2 |
| 362 | 19 | 200 | 11 | 200 | 7 | 1 |
| 363 | 19 | 200 | 11 | 200 | 7 | 2 |
| 364 | 19 | 200 | 11 | 200 | 7 | 3 |
| 365 | 19 | 200 | 11 | 200 | 7 | 2 |
| 366 | 19 | 200 | 11 | 200 | 7 | 1 |
| 367 | 19 | 200 | 11 | 200 | 7 | 1 |
| 368 | 13 | 248 | 15 | 45 | 15 | 1 |
| 369 | 12 | 76 | 16 | 83 | 9 | 3 |
| 370 | 19 | 200 | 11 | 200 | 7 | 2 |
| 371 | 15 | 367 | 20 | 147 | 13 | 3 |
| 372 | 19 | 200 | 11 | 200 | 7 | 1 |
| 373 | 15 | 1995 | 6 | 16 | 20 | 2 |
| 374 | 19 | 200 | 11 | 200 | 7 | 2 |
| 375 | 15 | 1954 | 5 | 22 | 21 | 2 |
| 376 | 12 | 50 | 20 | 90 | 12 | 3 |
| 377 | 17 | 918 | 15 | 107 | 12 | 2 |
| 378 | 19 | 200 | 11 | 200 | 7 | 3 |
| 379 | 8 | 285 | 9 | 912 | 20 | 2 |
| 380 | 5 | 16 | 16 | 22 | 6 | 2 |
| 381 | 19 | 200 | 11 | 200 | 7 | 2 |
| 382 | 19 | 200 | 11 | 200 | 7 | 2 |
| 383 | 19 | 200 | 11 | 200 | 7 | 1 |
| 384 | 19 | 200 | 11 | 200 | 7 | 4 |
| 385 | 19 | 200 | 11 | 200 | 7 | 1 |
| 386 | 19 | 200 | 11 | 200 | 7 | 2 |
| 387 | 19 | 200 | 11 | 200 | 7 | 1 |
| 388 | 6 | 117 | 12 | 68 | 11 | 2 |
| 389 | 22 | 78 | 18 | 625 | 19 | 4 |
| 390 | 9 | 84 | 21 | 59 | 17 | 4 |
| 391 | 19 | 200 | 11 | 200 | 7 | 2 |
| 392 | 19 | 200 | 11 | 200 | 7 | 1 |
| 393 | 19 | 200 | 11 | 200 | 7 | 3 |
| 394 | 19 | 200 | 11 | 200 | 7 | 1 |
| 395 | 19 | 200 | 11 | 200 | 7 | 2 |
| 396 | 21 | 298 | 10 | 30 | 5 | 2 |
| 397 | 19 | 200 | 11 | 200 | 7 | 3 |
| 398 | 10 | 100 | 15 | 17 | 24 | 2 |
| 399 | 19 | 200 | 11 | 200 | 7 | 3 |
| 400 | 19 | 200 | 11 | 200 | 7 | 2 |
| 401 | 19 | 200 | 11 | 200 | 7 | 3 |
| 402 | 19 | 200 | 11 | 200 | 7 | 1 |
| 403 | 19 | 200 | 11 | 200 | 7 | 2 |
| 404 | 19 | 200 | 11 | 200 | 7 | 2 |
| 405 | 19 | 200 | 11 | 200 | 7 | 2 |
| 406 | 19 | 200 | 11 | 200 | 7 | 3 |
| 407 | 21 | 193 | 5 | 938 | 20 | 1 |
| 408 | 19 | 200 | 11 | 200 | 7 | 4 |
| 409 | 19 | 200 | 11 | 200 | 7 | 2 |
| 410 | 19 | 200 | 11 | 200 | 7 | 2 |
| 411 | 8 | 230 | 13 | 70 | 14 | 2 |
| 412 | 14 | 86 | 14 | 88 | 13 | 1 |
| 413 | 12 | 1948 | 15 | 18 | 24 | 1 |
| 414 | 19 | 200 | 11 | 200 | 7 | 1 |
| 415 | 19 | 200 | 11 | 200 | 7 | 1 |
| 416 | 19 | 200 | 11 | 200 | 7 | 2 |
| 417 | 19 | 200 | 11 | 200 | 7 | 1 |
| 418 | 19 | 200 | 11 | 200 | 7 | 2 |
| 419 | 19 | 200 | 11 | 200 | 7 | 3 |
| 420 | 19 | 200 | 11 | 200 | 7 | 1 |
| 421 | 19 | 200 | 11 | 200 | 7 | 1 |
| 422 | 19 | 200 | 11 | 200 | 7 | 1 |
| 423 | 19 | 200 | 11 | 200 | 7 | 1 |
| 424 | 19 | 200 | 11 | 200 | 7 | 1 |
| 425 | 19 | 200 | 11 | 200 | 7 | 2 |
| 426 | 19 | 200 | 11 | 200 | 7 | 1 |
| 427 | 19 | 200 | 11 | 200 | 7 | 2 |
| 428 | 7 | 51 | 9 | 60 | 8 | 2 |
| 429 | 19 | 200 | 11 | 200 | 7 | 3 |
| 430 | 19 | 200 | 11 | 200 | 7 | 3 |
| 431 | 19 | 200 | 11 | 200 | 7 | 4 |
| 432 | 6 | 311 | 20 | 411 | 23 | 2 |
| 433 | 19 | 200 | 11 | 200 | 7 | 1 |
| 434 | 24 | 41 | 25 | 2048 | 10 | 4 |
| 435 | 14 | 1960 | 18 | 89 | 25 | 2 |
| 436 | 19 | 200 | 11 | 200 | 7 | 2 |
| 437 | 19 | 200 | 11 | 200 | 7 | 3 |
| 438 | 19 | 200 | 11 | 200 | 7 | 1 |
| 439 | 23 | 101 | 6 | 2022 | 24 | 1 |
| 440 | 19 | 200 | 11 | 200 | 7 | 1 |
| 441 | 19 | 200 | 11 | 200 | 7 | 3 |
| 442 | 19 | 200 | 11 | 200 | 7 | 4 |
| 443 | 19 | 200 | 11 | 200 | 7 | 1 |
| 444 | 13 | 1637 | 5 | 21 | 21 | 1 |
| 445 | 19 | 200 | 11 | 200 | 7 | 3 |
| 446 | 19 | 200 | 11 | 200 | 7 | 1 |
| 447 | 19 | 200 | 11 | 200 | 7 | 2 |
| 448 | 19 | 200 | 11 | 200 | 7 | 3 |
| 449 | 19 | 200 | 11 | 200 | 7 | 1 |
| 450 | 19 | 200 | 11 | 200 | 7 | 3 |
| 451 | 5 | 247 | 17 | 51 | 25 | 3 |
| 452 | 19 | 200 | 11 | 200 | 7 | 1 |
| 453 | 19 | 200 | 11 | 200 | 7 | 2 |
| 454 | 19 | 200 | 11 | 200 | 7 | 1 |
| 455 | 19 | 200 | 11 | 200 | 7 | 2 |
| 456 | 19 | 200 | 11 | 200 | 7 | 1 |
| 457 | 19 | 200 | 11 | 200 | 7 | 4 |
| 458 | 19 | 200 | 11 | 200 | 7 | 1 |
| 459 | 19 | 200 | 11 | 200 | 7 | 4 |
| 460 | 13 | 264 | 13 | 56 | 20 | 1 |
| 461 | 19 | 200 | 11 | 200 | 7 | 1 |
| 462 | 19 | 200 | 11 | 200 | 7 | 1 |
| 463 | 19 | 200 | 11 | 200 | 7 | 2 |
| 464 | 19 | 200 | 11 | 200 | 7 | 2 |
| 465 | 19 | 200 | 11 | 200 | 7 | 1 |
| 466 | 19 | 200 | 11 | 200 | 7 | 3 |
| 467 | 19 | 200 | 11 | 200 | 7 | 2 |
| 468 | 19 | 200 | 11 | 200 | 7 | 1 |
| 469 | 19 | 200 | 11 | 200 | 7 | 2 |
| 470 | 19 | 200 | 11 | 200 | 7 | 2 |
| 471 | 19 | 200 | 11 | 200 | 7 | 2 |
| 472 | 19 | 200 | 11 | 200 | 7 | 2 |
| 473 | 19 | 200 | 11 | 200 | 7 | 2 |
| 474 | 19 | 200 | 11 | 200 | 7 | 1 |
| 475 | 19 | 200 | 11 | 200 | 7 | 2 |
| 476 | 19 | 200 | 11 | 200 | 7 | 2 |
| 477 | 19 | 200 | 11 | 200 | 7 | 1 |
| 478 | 19 | 200 | 11 | 200 | 7 | 2 |
| 479 | 19 | 200 | 11 | 200 | 7 | 3 |
| 480 | 19 | 200 | 11 | 200 | 7 | 1 |
| 481 | 8 | 591 | 17 | 16 | 21 | 2 |
| 482 | 19 | 1464 | 6 | 42 | 25 | 1 |
| 483 | 19 | 200 | 11 | 200 | 7 | 1 |
| 484 | 14 | 1733 | 5 | 16 | 25 | 2 |
| 485 | 19 | 200 | 11 | 200 | 7 | 2 |
| 486 | 19 | 200 | 11 | 200 | 7 | 3 |
| 487 | 11 | 222 | 10 | 187 | 18 | 1 |
| 488 | 11 | 1872 | 6 | 16 | 20 | 2 |
| 489 | 19 | 200 | 11 | 200 | 7 | 2 |
| 490 | 19 | 200 | 11 | 200 | 7 | 1 |
| 491 | 19 | 200 | 11 | 200 | 7 | 1 |
| 492 | 19 | 200 | 11 | 200 | 7 | 2 |

| Row ID | linear_channels | linear_activation | linear_dropout_p | n_branched_layers | branched_channels | branched_activation |
|---|---|---|---|---|---|---|
| 1 | 1000 | ReLU | 0.3751173384603823 | 4 | 492 | ELU |
| 2 | 1000 | ReLU | 0.1625694487888689 | | | |
| 3 | 784 | ReLU | 0.5185600481782722 | | | |
| 4 | 1000 | ReLU | 0.5384056069932659 | 3 | 590 | ReLU6 |
| 5 | 1000 | ReLU | 0.05 | | | |
| 6 | 1000 | ReLU | 0.1254125501486332 | | | |
| 7 | 41 | ReLU | 0.3627219668454897 | | | |
| 8 | 1000 | ReLU | 0.4172768803695889 | 3 | 1023 | ELU |
| 9 | 928 | ReLU6 | 0.3061681593577892 | 1 | 170 | ReLU |
| 10 | 1000 | ReLU | 0.05 | | | |
| 11 | 1000 | ReLU | 0.0507988 | 4 | 1016 | ReLU6 |
| 12 | 1000 | ReLU | 0.05222308 | 3 | 1019 | ELU |
| 13 | 1000 | ReLU | 0.4857811657897323 | 2 | 731 | ReLU6 |
| 14 | 331 | ReLU6 | 0.4366309396025913 | 2 | 57 | ELU |
| 15 | 1000 | ReLU | 0.32336047 | 3 | 1021 | ReLU6 |
| 16 | 1000 | ReLU | 0.05 | 3 | 1024 | ReLU6 |
| 17 | 1000 | ReLU | 0.1317183817724209 | | | |
| 18 | 1000 | ReLU | 0.4657049394759744 | 5 | 617 | ReLU6 |
| 19 | 1000 | ReLU | 0.2499990502800381 | 3 | 1001 | ReLU6 |
| 20 | 1000 | ReLU | 0.05 | | | |
| 21 | 1000 | ReLU | 0.3648710904254541 | 2 | 1024 | ReLU6 |
| 22 | 1000 | ReLU | 0.1784180794489028 | 2 | 1024 | ReLU |
| 23 | 4096 | ReLU6 | 0.5635884976935613 | 1 | 16 | ReLU |
| 24 | 1000 | ReLU | 0.5013813357677481 | | | |
| 25 | 1000 | ReLU | 0.2125010238307914 | | | |
| 26 | 1000 | ReLU | 0.3438965527271348 | 5 | 598 | ReLU6 |
| 27 | 1000 | ReLU | 0.3834253482445222 | 3 | 558 | ReLU6 |
| 28 | 1000 | ReLU | 0.2006085650565899 | | | |
| 29 | 1000 | ReLU | 0.4638069959850518 | 4 | 680 | ReLU6 |
| 30 | 1000 | ReLU | 0.5437569560112212 | | | |
| 31 | 1000 | ReLU | 0.2279671 | | | |
| 32 | 1000 | ReLU | 0.05 | | | |
| 33 | 238 | ReLU6 | 0.6173961688315655 | 1 | 411 | ReLU6 |
| 34 | 1000 | ReLU | 0.2531060816423453 | 2 | 1023 | ReLU6 |
| 35 | 1000 | ReLU | 0.3902575797188325 | 3 | 1023 | ELU |
| 36 | 472 | ReLU | 0.05 | 2 | 772 | ReLU |
| 37 | 1000 | ReLU | 0.3672417052530124 | 2 | 1024 | ELU |
| 38 | 4094 | ReLU6 | 0.4967338154203973 | | | |
| 39 | 1000 | ReLU | 0.2627468380584772 | | | |
| 40 | 1000 | ReLU | 0.3831357066118553 | 2 | 528 | ELU |
| 41 | 368 | ReLU6 | 0.4723241437079822 | | | |
| 42 | 1000 | ReLU | 0.1495485550353351 | | | |
| 43 | 3742 | ReLU6 | 0.31587082 | 1 | 16 | ReLU6 |
| 44 | 1000 | ReLU | 0.05785734 | | | |
| 45 | 357 | ReLU | 0.5149715003359562 | | | |
| 46 | 72 | ELU | 0.4965029466342708 | | | |
| 47 | 1000 | ReLU | 0.1726104209368024 | | | |
| 48 | 1000 | ReLU | 0.4652904712729834 | 1 | 577 | ReLU6 |
| 49 | 1000 | ReLU | 0.1780788605295452 | | | |
| 50 | 1000 | ReLU | 0.5908324161669398 | 2 | 750 | ReLU |
| 51 | 1174 | ReLU6 | 0.5027651592866531 | | | |
| 52 | 1000 | ReLU | 0.1223558037239061 | 2 | 925 | ELU |
| 53 | 1000 | ReLU | 0.05 | | | |
| 54 | 1000 | ReLU | 0.05 | | | |
| 55 | 1000 | ReLU | 0.74822072 | | | |
| 56 | 1551 | ReLU6 | 0.6882370617283113 | 2 | 18 | ReLU |
| 57 | 1000 | ReLU | 0.2909322495485693 | | | |
| 58 | 1000 | ReLU | 0.07019088 | 3 | 1022 | ELU |
| 59 | 1000 | ReLU | 0.05029167 | 3 | 1019 | ReLU6 |
| 60 | 1000 | ReLU | 0.11637872 | 3 | 1016 | ReLU6 |
| 61 | 1000 | ReLU | 0.3519882478045145 | | | |
| 62 | 1000 | ReLU | 0.3988638987616758 | | | |
| 63 | 1000 | ReLU | 0.05 | 3 | 1011 | ReLU6 |
| 64 | 1000 | ReLU | 0.05012421 | | | |
| 65 | 1000 | ReLU | 0.39266065 | | | |
| 66 | 1000 | ReLU | 0.2896875 | 3 | 1019 | ReLU6 |
| 67 | 1000 | ReLU | 0.3611473573716173 | 3 | 1022 | ELU |
| 68 | 1000 | ReLU | 0.05 | | | |
| 69 | 1000 | ReLU | 0.05 | 2 | 1024 | ReLU |
| 70 | 1000 | ReLU | 0.05 | | | |
| 71 | 1000 | ReLU | 0.05 | 2 | 1024 | ELU |
| 72 | 17 | ReLU6 | 0.05047864 | | | |
| 73 | 109 | ReLU6 | 0.2089205584146037 | 1 | 211 | ReLU6 |
| 74 | 1030 | ELU | 0.60397016 | 3 | 29 | ReLU6 |
| 75 | 1000 | ReLU | 0.05 | | | |
| 76 | 1000 | ReLU | 0.2043437745847601 | 3 | 1024 | ReLU6 |
| 77 | 3862 | ReLU | 0.08641264 | | | |
| 78 | 1000 | ReLU | 0.05 | 3 | 976 | ReLU6 |
| 79 | 206 | ReLU6 | 0.06116112 | | | |
| 80 | 1000 | ReLU | 0.3352051113057671 | 2 | 1024 | ReLU6 |
| 81 | 1000 | ReLU | 0.07484842 | 3 | 1015 | ReLU6 |
| 82 | 1000 | ReLU | 0.07609943 | 4 | 1024 | ReLU6 |
| 83 | 1000 | ReLU | 0.08741988 | 4 | 998 | ELU |
| 84 | 403 | ReLU6 | 0.2747313692221874 | | | |
| 85 | 1000 | ReLU | 0.05005929 | 3 | 1024 | ReLU6 |
| 86 | 1000 | ReLU | 0.4486131618463236 | | | |
| 87 | 1000 | ReLU | 0.05 | | | |
| 88 | 1000 | ReLU | 0.06501265 | | | |
| 89 | 1000 | ReLU | 0.1236907084822667 | | | |
| 90 | 1000 | ReLU | 0.3999999999999999 | 3 | 520 | ELU |
| 91 | 161 | ReLU | 0.4071747801654821 | | | |
| 92 | 1000 | ReLU | 0.14576656 | | | |
| 93 | 1000 | ReLU | 0.05 | 2 | 1024 | ELU |
| 94 | 1000 | ReLU | 0.1622710153040233 | 3 | 1024 | ReLU6 |
| 95 | 1000 | ReLU | 0.2013230175562613 | 2 | 1024 | ELU |
| 96 | 995 | ReLU6 | 0.4953352685508664 | 1 | 16 | ReLU |
| 97 | 1000 | ReLU | 0.07512258 | 3 | 1023 | ELU |
| 98 | 1000 | ReLU | 0.08569846 | | | |
| 99 | 1000 | ReLU | 0.3051403825444136 | 2 | 1024 | ReLU |
| 100 | 1000 | ReLU | 0.5003974700337533 | | | |
| 101 | 1000 | ReLU | 0.20100977 | | | |
| 102 | 1000 | ReLU | 0.19179641 | 3 | 1024 | ReLU6 |
| 103 | 1000 | ReLU | 0.07226063 | 4 | 1010 | ReLU6 |
| 104 | 1000 | ReLU | 0.2032590177080204 | | | |
| 105 | 1000 | ReLU | 0.1408189552846019 | | | |
| 106 | 1000 | ReLU | 0.35312833 | 3 | 1023 | ELU |
| 107 | 1000 | ReLU | 0.05418374 | 4 | 1022 | ELU |
| 108 | 1000 | ReLU | 0.06525058 | | | |
| 109 | 1000 | ReLU | 0.07372703 | 5 | 1022 | ReLU6 |
| 110 | 1000 | ReLU | 0.75 | | | |
| 111 | 1000 | ReLU | 0.05 | 4 | 1022 | ReLU6 |
| 112 | 1000 | ReLU | 0.05 | | | |
| 113 | 1000 | ReLU | 0.5212736590524594 | 3 | 638 | ReLU |
| 114 | 1000 | ReLU | 0.0500711 | 2 | 1023 | ReLU6 |
| 115 | 1000 | ReLU | 0.1732766097627989 | | | |
| 116 | 1000 | ReLU | 0.1085843241777083 | | | |
| 117 | 1000 | ReLU | 0.34013933 | 4 | 612 | ELU |
| 118 | 1000 | ReLU | 0.3748455622533664 | | | |
| 119 | 1000 | ReLU | 0.07195771 | 3 | 1015 | ReLU6 |
| 120 | 1000 | ReLU | 0.4429848804779906 | 3 | 565 | ELU |
| 121 | 1000 | ReLU | 0.6113370341266768 | 5 | 1013 | ReLU6 |
| 122 | 1000 | ReLU | 0.3474723032069655 | 2 | 1024 | ReLU6 |
| 123 | 1000 | ReLU | 0.05270334 | 1 | 1024 | ReLU |
| 124 | 1000 | ReLU | 0.07663133 | 3 | 1011 | ELU |
| 125 | 1000 | ReLU | 0.05 | | | |
| 126 | 1000 | ReLU | 0.05 | | | |
| 127 | 1000 | ReLU | 0.4712354055915851 | | | |
| 128 | 1000 | ReLU | 0.05873024 | 3 | 1023 | ReLU |
| 129 | 1000 | ReLU | 0.0561075 | 3 | 1012 | ReLU6 |
| 130 | 1000 | ReLU | 0.59505379 | 2 | 755 | ReLU |
| 131 | 1000 | ReLU | 0.09558277 | 2 | 1019 | ReLU |
| 132 | 1000 | ReLU | 0.3464374350532389 | 3 | 1024 | ReLU6 |
| 133 | 1000 | ReLU | 0.1436253871763486 | | | |
| 134 | 1000 | ReLU | 0.07687712 | 3 | 1015 | ELU |
| 135 | 1147 | ReLU6 | 0.4834306442862945 | 1 | 160 | ReLU |
| 136 | 1000 | ReLU | 0.3267003765429385 | 3 | 535 | ReLU6 |
| 137 | 3779 | ReLU6 | 0.5119260883028861 | 2 | 24 | ReLU |
| 138 | 4095 | ReLU | 0.4060172563677027 | 3 | 24 | ELU |
| 139 | 4066 | ReLU6 | 0.5263606078969667 | 1 | 16 | ReLU |
| 140 | 2127 | ReLU | 0.3838333535286931 | 2 | 16 | ReLU |
| 141 | 1000 | ReLU | 0.05130757 | | | |
| 142 | 1000 | ReLU | 0.05081826 | 4 | 1023 | ReLU6 |
| 143 | 628 | ELU | 0.4822089735067524 | 2 | 79 | ELU |
| 144 | 1000 | ReLU | 0.05 | | | |
| 145 | 1000 | ReLU | 0.1837141293446532 | | | |
| 146 | 2770 | ReLU | 0.1807278232541296 | | | |
| 147 | 1000 | ReLU | 0.05130305 | | | |
| 148 | 4090 | ReLU6 | 0.3892075817166221 | 2 | 69 | ELU |
| 149 | 1000 | ReLU | 0.4314347021324866 | | | |
| 150 | 1000 | ReLU | 0.34804321 | 3 | 1022 | ELU |
| 151 | 1000 | ReLU | 0.4168213363016659 | 2 | 739 | ReLU6 |
| 152 | 835 | ReLU6 | 0.3539570820537278 | | | |
| 153 | 1000 | ReLU | 0.3665410848142287 | 2 | 1024 | ReLU6 |
| 154 | 587 | ReLU6 | 0.4128501504942942 | | | |
| 155 | 3692 | ReLU6 | 0.75 | | | |
| 156 | 1000 | ReLU | 0.4580205455247561 | | | |
| 157 | 1000 | ReLU | 0.1850768691263353 | | | |
| 158 | 1000 | ReLU | 0.2495422149496411 | 4 | 1022 | ReLU6 |
| 159 | 1000 | ReLU | 0.0603654 | 2 | 1014 | ReLU |
| 160 | 3163 | ReLU6 | 0.4745494279560511 | 1 | 17 | ELU |
| 161 | 3408 | ReLU6 | 0.3582814236840186 | 3 | 16 | ReLU |
| 162 | 16 | ReLU6 | 0.05 | | | |
| 163 | 1000 | ReLU | 0.08848714 | | | |
| 164 | 1000 | ReLU | 0.05 | | | |
| 165 | 1000 | ReLU | 0.2161266745401021 | | | |
| 166 | 449 | ReLU | 0.4583254834592234 | | | |
| 167 | 874 | ReLU6 | 0.3105444374360082 | 2 | 16 | ReLU6 |
| 168 | 3380 | ReLU6 | 0.5611969373168134 | 2 | 119 | ReLU |
| 169 | 473 | ReLU | 0.2912371487425154 | | | |
| 170 | 4096 | ReLU6 | 0.3856325584060475 | 2 | 31 | ELU |
| 171 | 4075 | ReLU6 | 0.32987711 | 1 | 16 | ReLU6 |
| 172 | 3248 | ReLU6 | 0.33242375 | 1 | 26 | ELU |
| 173 | 1000 | ReLU | 0.05086496 | 4 | 1024 | ReLU6 |
| 174 | 1000 | ReLU | 0.05132999 | 3 | 1023 | ReLU6 |
| 175 | 1000 | ReLU | 0.05 | | | |
| 176 | 1000 | ReLU | 0.1057295207427898 | 3 | 1009 | ReLU |
| 177 | 1000 | ReLU | 0.05 | | | |
| 178 | 1000 | ReLU | 0.3185524775557042 | 4 | 1013 | ReLU6 |
| 179 | 1000 | ReLU | 0.2098805337278699 | 3 | 987 | ELU |
| 180 | 1000 | ReLU | 0.07268184 | 4 | 1022 | ReLU6 |
| 181 | 1000 | ReLU | 0.3216328395162902 | 2 | 455 | ReLU6 |
| 182 | 4096 | ELU | 0.4015400047723333 | | | |
| 183 | 1000 | ReLU | 0.05 | | | |
| 184 | 1000 | ReLU | 0.3268112089784679 | 2 | 1024 | ReLU6 |
| 185 | 1000 | ReLU | 0.2946202004230411 | 4 | 1024 | ELU |
| 186 | 1000 | ReLU | 0.05 | | | |
| 187 | 53 | ReLU6 | 0.7257540059142138 | 2 | 39 | ELU |
| 188 | 1000 | ReLU | 0.1999506928590634 | | | |
| 189 | 1000 | ReLU | 0.08081133 | 3 | 1024 | ELU |
| 190 | 410 | ReLU6 | 0.3114514486831573 | 1 | 367 | ReLU |
| 191 | 305 | ReLU | 0.6022440634962009 | | | |
| 192 | 1000 | ReLU | 0.1319805080765406 | 3 | 1022 | ELU |
| 193 | 1000 | ReLU | 0.3166464018121155 | | | |
| 194 | 1000 | ReLU | 0.4862821259967815 | | | |
| 195 | 1000 | ReLU | 0.05 | | | |
| 196 | 1000 | ReLU | 0.5927777484994392 | | | |
| 197 | 1000 | ReLU | 0.2258590444593469 | | | |
| 198 | 1000 | ReLU | 0.3342764164649137 | 3 | 1024 | ReLU6 |
| 199 | 1000 | ReLU | 0.1866029190584974 | 4 | 1006 | ReLU |
| 200 | 1000 | ReLU | 0.4018067815243984 | 2 | 1024 | ReLU |
| 201 | 1000 | ReLU | 0.4284091636372312 | 3 | 1024 | ReLU6 |
| 202 | 1373 | ReLU6 | 0.33668181 | | | |
| 203 | 1000 | ReLU | 0.4530648436611826 | | | |
| 204 | 2151 | ReLU6 | 0.5525930465600614 | | | |
| 205 | 1000 | ReLU | 0.09372443 | 4 | 1017 | ELU |
| 206 | 1000 | ReLU | 0.05 | | | |
| 207 | 107 | ReLU | 0.3649311039442539 | | | |
| 208 | 1000 | ReLU | 0.5483220139951677 | 3 | 893 | ReLU6 |
| 209 | 1000 | ReLU | 0.09693187 | | | |
| 210 | 417 | ReLU6 | 0.6359846806326994 | 1 | 16 | ReLU |
| 211 | 1000 | ReLU | 0.3685237985577642 | 3 | 462 | ReLU6 |
| 212 | 1000 | ReLU | 0.1344421515643654 | 1 | 1024 | ReLU6 |
| 213 | 455 | ReLU6 | 0.5123614 | 3 | 515 | ELU |
| 214 | 1000 | ReLU | 0.1113402717401607 | | | |
| 215 | 1000 | ReLU | 0.37395296 | 3 | 514 | ReLU6 |
| 216 | 1000 | ReLU | 0.48146671 | | | |
| 217 | 1000 | ReLU | 0.06747324 | 4 | 1023 | ReLU6 |
| 218 | 1000 | ReLU | 0.11222836 | | | |
| 219 | 1000 | ReLU | 0.05 | | | |
| 220 | 1000 | ReLU | 0.1701952030033958 | | | |
| 221 | 1000 | ReLU | 0.3135092877378645 | 3 | 1024 | ReLU6 |
| 222 | 1000 | ReLU | 0.05 | | | |
| 223 | 1000 | ReLU | 0.4782827956809436 | 1 | 594 | ELU |
| 224 | 1000 | ReLU | 0.1731281296103286 | | | |
| 225 | 2568 | ReLU6 | 0.6370720082976709 | 1 | 274 | ReLU |
| 226 | 170 | ReLU6 | 0.4671726978680857 | | | |
| 227 | 2966 | ReLU6 | 0.5425578970235747 | 2 | 103 | ReLU |
| 228 | 1000 | ReLU | 0.05016112 | 3 | 962 | ReLU6 |
| 229 | 4096 | ReLU6 | 0.4872761687273896 | 2 | 88 | ReLU |
| 230 | 1137 | ReLU6 | 0.5757300375610255 | 1 | 332 | ReLU6 |
| 231 | 1000 | ReLU | 0.2170117694479105 | 3 | 1023 | ReLU |
| 232 | 1000 | ReLU | 0.0807837 | 5 | 1021 | ReLU6 |
| 233 | 1000 | ReLU | 0.05607979 | 2 | 1022 | ELU |
| 234 | 1083 | ReLU6 | 0.56818685 | 2 | 16 | ELU |
| 235 | 1000 | ReLU | 0.1506035707291938 | 3 | 1024 | ELU |
| 236 | 1000 | ReLU | 0.06935986 | 4 | 1024 | ReLU6 |
| 237 | 1000 | ReLU | 0.06751821 | | | |
| 238 | 1000 | ReLU | 0.1079725486391216 | | | |
| 239 | 1000 | ReLU | 0.05 | 2 | 1024 | ReLU6 |
| 240 | 1000 | ReLU | 0.4729820066074196 | 4 | 1007 | ReLU6 |
| 241 | 330 | ReLU6 | 0.4725301770941048 | 1 | 265 | ELU |
| 242 | 1000 | ReLU | 0.1985422914905666 | 2 | 1024 | ReLU |
| 243 | 1000 | ReLU | 0.0544142 | 4 | 1021 | ReLU |
| 244 | 2620 | ReLU6 | 0.4892106200680247 | 1 | 17 | ReLU6 |
| 245 | 701 | ReLU6 | 0.4663603627155543 | 1 | 569 | ReLU6 |
| 246 | 1000 | ReLU | 0.4271783103692763 | 2 | 1017 | ReLU6 |
| 247 | 212 | ELU | 0.75 | 1 | 203 | ReLU6 |
| 248 | 1000 | ReLU | 0.1137759669924336 | 3 | 1021 | ELU |
| 249 | 717 | ReLU6 | 0.39595536 | 2 | 429 | ELU |
| 250 | 40 | ReLU6 | 0.050598 | | | |
| 251 | 1000 | ReLU | 0.4603215878020662 | 3 | 1024 | ReLU6 |
| 252 | 23 | ReLU6 | 0.05 | | | |
| 253 | 1000 | ReLU | 0.05825524 | | | |
| 254 | 50 | ReLU | 0.5203827124733706 | | | |
| 255 | 714 | ReLU6 | 0.6598202635345763 | | | |
| 256 | 1000 | ReLU | 0.05020158 | 3 | 1022 | ReLU6 |
| 257 | 3123 | ReLU6 | 0.08001663 | 2 | 16 | ReLU6 |
| 258 | 702 | ReLU | 0.4711334065373665 | | | |
| 259 | 1000 | ReLU | 0.5313218000778814 | | | |
| 260 | 1000 | ReLU | 0.05 | | | |
| 261 | 799 | ReLU | 0.45708482 | | | |
| 262 | 1000 | ReLU | 0.1629465696229217 | | | |
| 263 | 1000 | ReLU | 0.05 | | | |
| 264 | 2676 | ReLU | 0.4902279109627583 | 2 | 76 | ReLU |
| 265 | 2679 | ReLU6 | 0.36187629 | 1 | 30 | ReLU |
| 266 | 1148 | ReLU6 | 0.2129575103573911 | | | |
| 267 | 1000 | ReLU | 0.05 | 2 | 1017 | ELU |
| 268 | 1000 | ReLU | 0.1403951236467163 | | | |
| 269 | 71 | ReLU | 0.1387917562642852 | | | |
| 270 | 48 | ReLU6 | 0.1720283160102884 | | | |
| 271 | 1000 | ReLU | 0.5516470708860638 | 2 | 646 | ReLU6 |
| 272 | 1000 | ReLU | 0.05009959 | 3 | 988 | ReLU6 |
| 273 | 182 | ReLU6 | 0.3356216551939365 | | | |
| 274 | 1000 | ReLU | 0.2360127883310405 | | | |
| 275 | 1000 | ReLU | 0.2284422754580748 | | | |
| 276 | 1000 | ReLU | 0.3994245653292712 | 2 | 1024 | ReLU6 |
| 277 | 691 | ReLU6 | 0.1855110486497475 | 2 | 279 | ELU |
| 278 | 1000 | ReLU | 0.4402627755451269 | | | |
| 279 | 3283 | ReLU6 | 0.2646310949426411 | 4 | 37 | ReLU |
| 280 | 1000 | ReLU | 0.05019002 | | | |
| 281 | 1000 | ReLU | 0.06398718 | 3 | 1023 | ReLU6 |
| 282 | 1000 | ReLU | 0.1182914884236928 | 4 | 1024 | ReLU6 |
| 283 | 1000 | ReLU | 0.4319634827078589 | 3 | 1024 | ELU |
| 284 | 1000 | ReLU | 0.05 | 2 | 1014 | ELU |
| 285 | 232 | ReLU6 | 0.3671279741388354 | | | |
| 286 | 1000 | ReLU | 0.05095882 | 2 | 958 | ReLU6 |
| 287 | 1000 | ReLU | 0.05 | 2 | 784 | ReLU6 |
| 288 | 1000 | ReLU | 0.4945858040944448 | 4 | 1023 | ReLU |
| 289 | 1000 | ReLU | 0.3105096800387474 | | | |
| 290 | 1000 | ReLU | 0.08359481 | | | |
| 291 | 672 | ReLU6 | 0.74806303 | 3 | 70 | ReLU |
| 292 | 1000 | ReLU | 0.4225690361797186 | 2 | 1009 | ReLU6 |
| 293 | 1000 | ReLU | 0.3213425923252694 | 2 | 1024 | ReLU6 |
| 294 | 387 | ReLU6 | 0.2148951210600898 | | | |
| 295 | 1000 | ReLU | 0.4437763033975703 | 3 | 1024 | ReLU6 |
| 296 | 1000 | ReLU | 0.1562000803218945 | | | |
| 297 | 1000 | ReLU | 0.05104046 | 2 | 1024 | ReLU6 |
| 298 | 1000 | ReLU | 0.05 | | | |
| 299 | 1000 | ReLU | 0.1366427291261992 | | | |
| 300 | 1000 | ReLU | 0.4030944837977042 | 2 | 1021 | ReLU6 |
| 301 | 1000 | ReLU | 0.3645129321700112 | 3 | 1022 | ELU |
| 302 | 1000 | ReLU | 0.05 | | | |
| 303 | 1000 | ReLU | 0.1113065947621472 | | | |
| 304 | 1000 | ReLU | 0.4109635833391865 | 3 | 439 | ReLU6 |
| 305 | 3339 | ReLU6 | 0.4627822199185563 | 4 | 16 | ReLU6 |
| 306 | 2084 | ReLU6 | 0.4354867847225696 | | | |
| 307 | 1000 | ReLU | 0.4200006678490979 | 3 | 757 | ReLU6 |
| 308 | 1131 | ReLU | 0.6006825114385437 | 2 | 67 | ReLU6 |
| 309 | 1000 | ReLU | 0.05156137 | 3 | 1016 | ELU |
| 310 | 1000 | ReLU | 0.3721202419400265 | 3 | 1020 | ELU |
| 311 | 1000 | ReLU | 0.4657784961580156 | 3 | 941 | ReLU6 |
| 312 | 845 | ReLU6 | 0.4423567464014015 | 3 | 401 | ReLU |
| 313 | 1000 | ReLU | 0.09980571 | 3 | 1014 | ELU |
| 314 | 1598 | ELU | 0.05 | | | |
| 315 | 1000 | ReLU | 0.4213939777736042 | 4 | 1014 | ReLU |
| 316 | 1000 | ReLU | 0.05276103 | | | |
| 317 | 98 | ReLU6 | 0.05 | | | |
| 318 | 1000 | ReLU | 0.05086819 | 3 | 996 | ReLU6 |
| 319 | 1000 | ReLU | 0.2003747881999175 | 2 | 1024 | ELU |
| 320 | 1000 | ReLU | 0.0517968 | 3 | 1019 | ReLU6 |
| 321 | 1000 | ReLU | 0.2266720282659386 | 3 | 1024 | ReLU6 |
| 322 | 2937 | ReLU6 | 0.3421290217423798 | 2 | 32 | ELU |
| 323 | 1000 | ReLU | 0.19782511 | | | |
| 324 | 1000 | ReLU | 0.2485570751121776 | 3 | 1006 | ELU |
| 325 | 1000 | ReLU | 0.3681811908453477 | 2 | 1023 | ELU |
| 326 | 1000 | ReLU | 0.2551403663425927 | | | |
| 327 | 1000 | ReLU | 0.14607766 | 3 | 974 | ReLU6 |
| 328 | 893 | ReLU6 | 0.4388237135551531 | | | |
| 329 | 1000 | ReLU | 0.08602462 | 4 | 1014 | ReLU6 |
| 330 | 1000 | ReLU | 0.05 | | | |
| 331 | 1000 | ReLU | 0.1799060974377135 | | | |
| 332 | 1000 | ReLU | 0.4854940735893416 | 4 | 1024 | ELU |
| 333 | 1000 | ReLU | 0.4022736695671571 | 1 | 483 | ELU |
| 334 | 1000 | ReLU | 0.4509914089516004 | 4 | 753 | ReLU6 |
| 335 | 3131 | ReLU6 | 0.2878705512969984 | 1 | 278 | ELU |
| 336 | 700 | ELU | 0.7302473096405017 | 2 | 16 | ReLU |
| 337 | 1000 | ReLU | 0.4995712361848424 | 3 | 1024 | ELU |
| 338 | 1000 | ReLU | 0.3547316753894512 | 2 | 1024 | ReLU |
| 339 | 4096 | ReLU | 0.4385950787435006 | | | |
| 340 | 1000 | ReLU | 0.4661509161920739 | 3 | 926 | ReLU6 |
| 341 | 1000 | ReLU | 0.4297085765152351 | 3 | 786 | ELU |
| 342 | 16 | ReLU | 0.05 | | | |
| 343 | 1000 | ReLU | 0.05088077 | 4 | 1024 | ReLU6 |
| 344 | 1051 | ReLU | 0.5178541921641974 | | | |
| 345 | 1000 | ReLU | 0.09925 | 3 | 1023 | ReLU6 |
| 346 | 1000 | ReLU | 0.05 | | | |
| 347 | 1000 | ReLU | 0.1764338832998441 | | | |
| 348 | 487 | ReLU6 | 0.1956537254407229 | 2 | 38 | ReLU |
| 349 | 81 | ReLU6 | 0.1891552680964646 | | | |
| 350 | 1000 | ReLU | 0.0514382 | 5 | 1014 | ELU |
| 351 | 1000 | ReLU | 0.2956350940197257 | 2 | 1017 | ReLU |
| 352 | 1000 | ReLU | 0.05 | | | |
| 353 | 162 | ReLU6 | 0.58646978 | 1 | 245 | ReLU6 |
| 354 | 1000 | ReLU | 0.05 | 4 | 1024 | ELU |
| 355 | 3749 | ReLU6 | 0.40805227 | 3 | 16 | ELU |
| 356 | 3172 | ReLU6 | 0.4669993045250104 | 2 | 70 | ELU |
| 357 | 1000 | ReLU | 0.4466828731150701 | 3 | 629 | ReLU6 |
| 358 | 1000 | ReLU | 0.05 | | | |
| 359 | 96 | ReLU6 | 0.05 | 1 | 63 | ELU |
| 360 | 1000 | ReLU | 0.4252080858998709 | 2 | 1024 | ELU |
| 361 | 3392 | ReLU6 | 0.6824504188694449 | 2 | 16 | ReLU |
| 362 | 1000 | ReLU | 0.4460869034441748 | 2 | 1024 | ReLU6 |
| 363 | 1000 | ReLU | 0.05 | 3 | 1024 | ReLU6 |
| 364 | 1000 | ReLU | 0.05 | | | |
| 365 | 1000 | ReLU | 0.06343189 | | | |
| 366 | 1000 | ReLU | 0.3393304776817132 | 3 | 1024 | ELU |
| 367 | 1000 | ReLU | 0.1180851001311002 | | | |
| 368 | 665 | ReLU6 | 0.3960454086364462 | 2 | 169 | ELU |
| 369 | 437 | ELU | 0.5348908468541166 | | | |
| 370 | 1000 | ReLU | 0.05 | 4 | 1019 | ReLU6 |
| 371 | 411 | ELU | 0.6241094076772719 | 1 | 549 | ELU |
| 372 | 1000 | ReLU | 0.08865773 | | | |
| 373 | 554 | ReLU | 0.3949227242792817 | | | |
| 374 | 1000 | ReLU | 0.4486278247609791 | 2 | 714 | ReLU6 |
| 375 | 1626 | ReLU | 0.4442110644484177 | | | |
| 376 | 1200 | ReLU | 0.4012830953550043 | | | |
| 377 | 3876 | ReLU6 | 0.5073003175147818 | 2 | 16 | ReLU |
| 378 | 1000 | ReLU | 0.05 | | | |
| 379 | 3932 | ReLU6 | 0.3216109194010267 | 1 | 65 | ReLU6 |
| 380 | 1292 | ReLU6 | 0.75 | | | |
| 381 | 1000 | ReLU | 0.2194382 | 2 | 1024 | ReLU6 |
| 382 | 1000 | ReLU | 0.4271159811730626 | 2 | 859 | ReLU6 |
| 383 | 1000 | ReLU | 0.7075288215486202 | 3 | 1024 | ReLU6 |
| 384 | 1000 | ReLU | 0.07413487 | 5 | 1024 | ReLU6 |
| 385 | 1000 | ReLU | 0.05 | | | |
| 386 | 1000 | ReLU | 0.3870371141600563 | 2 | 723 | ReLU6 |
| 387 | 1000 | ReLU | 0.05160227 | 3 | 1022 | ELU |
| 388 | 913 | ReLU | 0.3280954343249836 | 1 | 169 | ReLU |
| 389 | 288 | ReLU6 | 0.6076678795816601 | 2 | 62 | ELU |
| 390 | 1675 | ELU | 0.2374208549208644 | | | |
| 391 | 1000 | ReLU | 0.7489930735760684 | | | |
| 392 | 1000 | ReLU | 0.1849604698272525 | | | |
| 393 | 1000 | ReLU | 0.08787256 | 4 | 1024 | ELU |
| 394 | 1000 | ReLU | 0.3551515282950795 | 5 | 1024 | ReLU6 |
| 395 | 1000 | ReLU | 0.2740128966576018 | 5 | 1024 | ReLU6 |
| 396 | 4089 | ReLU | 0.46425008 | | | |
| 397 | 1000 | ReLU | 0.05012944 | | | |
| 398 | 96 | ReLU6 | 0.1065179699851303 | | | |
| 399 | 1000 | ReLU | 0.6062560925838472 | | | |
| 400 | 1000 | ReLU | 0.1114444426517973 | 3 | 1024 | ReLU |
| 401 | 1000 | ReLU | 0.05215168 | 4 | 1024 | ReLU6 |
| 402 | 1000 | ReLU | 0.5525445812830517 | 2 | 746 | ReLU |
| 403 | 1000 | ReLU | 0.4144793388943764 | 3 | 1004 | ReLU6 |
| 404 | 1000 | ReLU | 0.4298079425522201 | 4 | 644 | ELU |
| 405 | 1000 | ReLU | 0.4379492218069534 | 4 | 1022 | ELU |
| 406 | 1000 | ReLU | 0.09774057 | 2 | 1024 | ReLU |
| 407 | 2374 | ReLU | 0.4248926726335804 | 1 | 16 | ELU |
| 408 | 1000 | ReLU | 0.05 | 3 | 1011 | ReLU6 |
| 409 | 1000 | ReLU | 0.32427092 | 3 | 1024 | ELU |
| 410 | 1000 | ReLU | 0.05 | 4 | 1024 | ReLU6 |
| 411 | 1393 | ReLU6 | 0.3462570409085128 | 2 | 131 | ELU |
| 412 | 355 | ReLU | 0.5132710835686372 | | | |
| 413 | 1493 | ReLU6 | 0.4672910744930151 | 2 | 16 | ELU |
| 414 | 1000 | ReLU | 0.09308173 | | | |
| 415 | 1000 | ReLU | 0.6935484689542757 | 1 | 897 | ReLU6 |
| 416 | 1000 | ReLU | 0.3868023750319643 | 4 | 1024 | ReLU6 |
| 417 | 1000 | ReLU | 0.05 | | | |
| 418 | 1000 | ReLU | 0.3749653 | 2 | 997 | ReLU6 |
| 419 | 1000 | ReLU | 0.05058104 | 3 | 1024 | ReLU6 |
| 420 | 1000 | ReLU | 0.05450856 | | | |
| 421 | 1000 | ReLU | 0.2357298345549947 | | | |
| 422 | 1000 | ReLU | 0.07693084 | 5 | 1023 | ELU |
| 423 | 1000 | ReLU | 0.07476299 | 3 | 1024 | ELU |
| 424 | 1000 | ReLU | 0.17257128 | | | |
| 425 | 1000 | ReLU | 0.5197466847206341 | 2 | 669 | ReLU |
| 426 | 1000 | ReLU | 0.10279858 | | | |
| 427 | 1000 | ReLU | 0.05 | | | |
| 428 | 391 | ReLU | 0.2309472 | 2 | 620 | ReLU |
| 429 | 1000 | ReLU | 0.05 | 3 | 1020 | ReLU6 |
| 430 | 1000 | ReLU | 0.5048140313224942 | 3 | 474 | ReLU6 |
| 431 | 1000 | ReLU | 0.05 | 4 | 1015 | ReLU6 |
| 432 | 21 | ReLU6 | 0.15655426 | | | |
| 433 | 1000 | ReLU | 0.05 | | | |
| 434 | 17 | ReLU | 0.7462346847013794 | 2 | 32 | ReLU6 |
| 435 | 4096 | ReLU6 | 0.2266502321079893 | 2 | 20 | ReLU6 |
| 436 | 1000 | ReLU | 0.06466727 | 2 | 977 | ELU |
| 437 | 1000 | ReLU | 0.05 | | | |
| 438 | 1000 | ReLU | 0.07121229 | | | |
| 439 | 1997 | ReLU6 | 0.1655865244215542 | 1 | 100 | ReLU |
| 440 | 1000 | ReLU | 0.1373528409459063 | | | |
| 441 | 1000 | ReLU | 0.2725437420726872 | 3 | 1022 | ReLU6 |
| 442 | 1000 | ReLU | 0.08141877 | 3 | 1012 | ReLU6 |
| 443 | 1000 | ReLU | 0.1049617766475056 | | | |
| 444 | 978 | ReLU6 | 0.5480492841863563 | | | |
| 445 | 1000 | ReLU | 0.05 | | | |
| 446 | 1000 | ReLU | 0.05 | | | |
| 447 | 1000 | ReLU | 0.07698057 | 3 | 1015 | ReLU6 |
| 448 | 1000 | ReLU | 0.05 | 3 | 1024 | ReLU |
| 449 | 1000 | ReLU | 0.1416395372974489 | | | |
| 450 | 1000 | ReLU | 0.05666705 | 3 | 1023 | ELU |
| 451 | 29 | ReLU6 | 0.05607526 | | | |
| 452 | 1000 | ReLU | 0.2603618159875546 | | | |
| 453 | 1000 | ReLU | 0.4399878916152405 | | | |
| 454 | 1000 | ReLU | 0.1589603333872718 | | | |
| 455 | 1000 | ReLU | 0.3270447985798737 | 3 | 1024 | ReLU6 |
| 456 | 1000 | ReLU | 0.05044406 | | | |
| 457 | 1000 | ReLU | 0.5519369788388859 | | | |
| 458 | 1000 | ReLU | 0.05 | | | |
| 459 | 1000 | ReLU | 0.05 | 4 | 1021 | ReLU6 |
| 460 | 1464 | ELU | 0.3407182251637595 | 2 | 16 | ReLU |
| 461 | 1000 | ReLU | 0.05 | | | |
| 462 | 1000 | ReLU | 0.065797 | | | |
| 463 | 1000 | ReLU | 0.5673561873501224 | 5 | 922 | ELU |
| 464 | 1000 | ReLU | 0.4292348601392928 | 3 | 764 | ReLU6 |
| 465 | 1000 | ReLU | 0.1005311477522733 | | | |
| 466 | 1000 | ReLU | 0.4259109158153608 | | | |
| 467 | 1000 | ReLU | 0.17388594 | 2 | 1024 | ReLU6 |
| 468 | 1000 | ReLU | 0.30012676 | | | |
| 469 | 1000 | ReLU | 0.1361555737653917 | 2 | 565 | ReLU6 |
| 470 | 1000 | ReLU | 0.5186868140627227 | 2 | 829 | ReLU6 |
| 471 | 1000 | ReLU | 0.05 | 5 | 1020 | ReLU6 |
| 472 | 1000 | ReLU | 0.4046118209445426 | | | |
| 473 | 1000 | ReLU | 0.40776645 | 4 | 1002 | ReLU6 |
| 474 | 1000 | ReLU | 0.08843638 | | | |
| 475 | 1000 | ReLU | 0.07245094 | 4 | 1010 | ReLU6 |
| 476 | 1000 | ReLU | 0.4071261585378397 | 4 | 535 | ELU |
| 477 | 1000 | ReLU | 0.1698483933526843 | | | |
| 478 | 1000 | ReLU | 0.05106901 | | | |
| 479 | 1000 | ReLU | 0.4647512828363357 | | | |
| 480 | 1000 | ReLU | 0.2794694401066447 | | | |
| 481 | 778 | ReLU6 | 0.5236716760104897 | 1 | 25 | ReLU |
| 482 | 1099 | ELU | 0.6577884082411262 | | | |
| 483 | 1000 | ReLU | 0.05 | 3 | 1024 | ReLU |
| 484 | 822 | ReLU6 | 0.4606846776667519 | | | |
| 485 | 1000 | ReLU | 0.3982296132119892 | | | |
| 486 | 1000 | ReLU | 0.4394602964737412 | 3 | 869 | ELU |
| 487 | 4096 | ReLU6 | 0.3716885445662743 | 2 | 31 | ReLU |
| 488 | 139 | ReLU6 | 0.6907705343735194 | | | |
| 489 | 1000 | ReLU | 0.4112712887806351 | | | |
| 490 | 1000 | ReLU | 0.09402108 | 2 | 1012 | ReLU6 |

| | | | |
|---|---|---|---|
| 491 | 1000 | ReLU | 0.3390923214470112 |
| 492 | 1000 | ReLU | 0.1399806085307181 |

| Row ID | branched_dropout_p | loss_criterion | parent_weights | frozen_epochs | model_module | graph_module |
|---|---|---|---|---|---|---|
| 1 | 0.39883856 | MSEKLmixed | gs://syrgoth/my- | 35 | BassetBranched | CNNTransferLearning |
| | | | model.epoch_5- | | | |
| | | | step_19885.pkl | | | |
| 2 | | L1KLmixed | gs://syrgoth/my- | 20 | BassetVL | CNNTransferLearning |
| | | | model.epoch_5- | | | |
| | | | step_19885.pkl | | | |
| 3 | | L1KLmixed | | | BassetVL | CNNBasicTraining |
| 4 | 0.2016548939078657 | L1KLmixed | gs://syrgoth/my- | 48 | BassetBranched | CNNTransferLearning |
| | | | model.epoch_5- | | | |
| | | | step_19885.pkl | | | |
| 5 | | L1KLmixed | gs://syrgoth/my- | 49 | BassetVL | CNNTransferLearning |
| | | | model.epoch_5- | | | |
| | | | step_19885.pkl | | | |
| 6 | | L1KLmixed | gs://syrgoth/my- | 55 | BassetVL | CNNTransferLearning |
| | | | model.epoch_5- | | | |
| | | | step_19885.pkl | | | |
| 7 | | L1KLmixed | | | BassetVL | CNNBasicTraining |
| 8 | 0.4455681237419353 | L1KLmixed | gs://syrgoth/my- | 24 | BassetBranched | CNNTransferLearning |
| | | | model.epoch_5- | | | |
| | | | step_19885.pkl | | | |
| 9 | 0.1384091850680277 | L1KLmixed | | | BassetBranched | CNNTransferLearning |
| 10 | | L1KLmixed | gs://syrgoth/my- | 46 | BassetVL | CNNTransferLearning |
| | | | model.epoch_5- | | | |
| | | | step_19885.pkl | | | |
| 11 | 0.3811294556270088 | L1KLmixed | gs://syrgoth/my- | 33 | BassetBranched | CNNTransferLearning |
| | | | model.epoch_5- | | | |
| | | | step_19885.pkl | | | |
| 12 | 0.5051434644305528 | L1KLmixed | gs://syrgoth/my- | 31 | BassetBranched | CNNTransferLearning |
| | | | model.epoch_5- | | | |
| | | | step_19885.pkl | | | |
| 13 | 0.3632227826345389 | MSEKLmixed | gs://syrgoth/my- | 42 | BassetBranched | CNNTransferLearning |
| | | | model.epoch_5- | | | |
| | | | step_19885.pkl | | | |
| 14 | 0.1522463672538527 | MSEKLmixed | | | BassetBranched | CNNBasicTraining |
| 15 | 0.5459701742768861 | L1KLmixed | gs://syrgoth/my- | 37 | BassetBranched | CNNTransferLearning |
| | | | model.epoch_5- | | | |
| | | | step_19885.pkl | | | |
| 16 | 0.48884509 | L1KLmixed | gs://syrgoth/my- | 22 | BassetBranched | CNNTransferLearning |
| | | | model.epoch_5- | | | |
| | | | step_19885.pkl | | | |
| 17 | | L1KLmixed | gs://syrgoth/my- | 52 | BassetVL | CNNTransferLearning |
| | | | model.epoch_5- | | | |
| | | | step_19885.pkl | | | |
| 18 | 0.2924177582903065 | L1KLmixed | gs://syrgoth/my- | 38 | BassetBranched | CNNTransferLearning |
| | | | model.epoch_5- | | | |
| | | | step_19885.pkl | | | |
| 19 | 0.3543928831110102 | L1KLmixed | gs://syrgoth/my- | 27 | BassetBranched | CNNTransferLearning |
| | | | model.epoch_5- | | | |
| | | | step_19885.pkl | | | |
| 20 | | L1KLmixed | gs://syrgoth/my- | 46 | BassetVL | CNNTransferLearning |
| | | | model.epoch_5- | | | |
| | | | step_19885.pkl | | | |
| 21 | 0.3455186670640106 | L1KLmixed | gs://syrgoth/my- | 16 | BassetBranched | CNNTransferLearning |
| | | | model.epoch_5- | | | |
| | | | step_19885.pkl | | | |
| 22 | 0.4370676068779432 | L1KLmixed | gs://syrgoth/my- | 24 | BassetBranched | CNNTransferLearning |
| | | | model.epoch_5- | | | |
| | | | step_19885.pkl | | | |
| 23 | 0.05 | MSEKLmixed | | | BassetBranched | CNNBasicTraining |
| 24 | | L1KLmixed | gs://syrgoth/my- | 38 | BassetVL | CNNTransferLearning |
| | | | model.epoch_5- | | | |
| | | | step_19885.pkl | | | |
| 25 | | L1KLmixed | gs://syrgoth/my- | 46 | BassetVL | CNNTransferLearning |
| | | | model.epoch_5- | | | |
| | | | step_19885.pkl | | | |
| 26 | 0.3829009658825631 | L1KLmixed | gs://syrgoth/my- | 37 | BassetBranched | CNNTransferLearning |
| | | | model.epoch_5- | | | |
| | | | step_19885.pkl | | | |
| 27 | 0.6523004207821921 | MSEKLmixed | gs://syrgoth/my- | 39 | BassetBranched | CNNTransferLearning |
| | | | model.epoch_5- | | | |
| | | | step_19885.pkl | | | |
| 28 | | L1KLmixed | gs://syrgoth/my- | 53 | BassetVL | CNNTransferLearning |
| | | | model.epoch_5- | | | |
| | | | step_19885.pkl | | | |
| 29 | 0.41952019 | MSEKLmixed | gs://syrgoth/my- | 38 | BassetBranched | CNNTransferLearning |
| | | | model.epoch_5- | | | |
| | | | step_19885.pkl | | | |
| 30 | | L1KLmixed | gs://syrgoth/my- | 55 | BassetVL | CNNTransferLearning |
| | | | model.epoch_5- | | | |
| | | | step_19885.pkl | | | |
| 31 | | L1KLmixed | gs://syrgoth/my- | 47 | BassetVL | CNNTransferLearning |
| | | | model.epoch_5- | | | |
| | | | step_19885.pkl | | | |
| 32 | | L1KLmixed | gs://syrgoth/my- | 60 | BassetVL | CNNTransferLearning |
| | | | model.epoch_5- | | | |
| | | | step_19885.pkl | | | |
| 33 | 0.4227020529346248 | MSEKLmixed | | | BassetBranched | CNNBasicTraining |
| 34 | 0.4032213119950632 | L1KLmixed | gs://syrgoth/my- | 32 | BassetBranched | CNNTransferLearning |
| | | | model.epoch_5- | | | |
| | | | step_19885.pkl | | | |
| 35 | 0.4703388610685092 | L1KLmixed | gs://syrgoth/my- | 37 | BassetBranched | CNNTransferLearning |
| | | | model.epoch_5- | | | |
| | | | step_19885.pkl | | | |
| 36 | 0.3461697948838907 | MSEKLmixed | | | BassetBranched | CNNBasicTraining |
| 37 | 0.4278172962346585 | L1KLmixed | gs://syrgoth/my- | 29 | BassetBranched | CNNTransferLearning |
| | | | model.epoch_5- | | | |
| | | | step_19885.pkl | | | |
| 38 | | L1KLmixed | | | BassetVL | CNNBasicTraining |
| 39 | | L1KLmixed | gs://syrgoth/my- | 36 | BassetVL | CNNTransferLearning |
| | | | model.epoch_5- | | | |
| | | | step_19885.pkl | | | |
| 40 | 0.38450757 | MSEKLmixed | gs://syrgoth/my- | 13 | BassetBranched | CNNTransferLearning |
| | | | model.epoch_5- | | | |
| | | | step_19885.pkl | | | |
| 41 | | L1KLmixed | | | BassetVL | CNNBasicTraining |
| 42 | | L1KLmixed | gs://syrgoth/my- | 46 | BassetVL | CNNTransferLearning |
| | | | model.epoch_5- | | | |
| | | | step_19885.pkl | | | |
| 43 | 0.05241947 | L1KLmixed | | | BassetBranched | CNNBasicTraining |
| 44 | | L1KLmixed | gs://syrgoth/my- | 46 | BassetVL | CNNTransferLearning |
| | | | model.epoch_5- | | | |
| | | | step_19885.pkl | | | |
| 45 | | L1KLmixed | | | BassetVL | CNNBasicTraining |
| 46 | | MSEKLmixed | | | BassetVL | CNNBasicTraining |
| 47 | | L1KLmixed | gs://syrgoth/my- | 41 | BassetVL | CNNTransferLearning |
| | | | model.epoch_5- | | | |
| | | | step_19885.pkl | | | |
| 48 | 0.4194859104331949 | L1KLmixed | gs://syrgoth/my- | 40 | BassetBranched | CNNTransferLearning |
| | | | model.epoch_5- | | | |
| | | | step_19885.pkl | | | |
| 49 | | L1KLmixed | gs://syrgoth/my- | 49 | BassetVL | CNNTransferLearning |
| | | | model.epoch_5- | | | |
| | | | step_19885.pkl | | | |
| 50 | 0.3407393029263886 | L1KLmixed | gs://syrgoth/my- | 31 | BassetBranched | CNNTransferLearning |
| | | | model.epoch_5- | | | |
| | | | step_19885.pkl | | | |
| 51 | | L1KLmixed | | | BassetVL | CNNBasicTraining |
| 52 | 0.4481088019821662 | L1KLmixed | gs://syrgoth/my- | 31 | BassetBranched | CNNTransferLearning |
| | | | model.epoch_5- | | | |
| | | | step_19885.pkl | | | |
| 53 | | L1KLmixed | gs://syrgoth/my- | 60 | BassetVL | CNNTransferLearning |
| | | | model.epoch_5- | | | |
| | | | step_19885.pkl | | | |
| 54 | | L1KLmixed | gs://syrgoth/my- | 48 | BassetVL | CNNTransferLearning |
| | | | model.epoch_5- | | | |
| | | | step_19885.pkl | | | |
| 55 | | L1KLmixed | gs://syrgoth/my- | 2 | BassetVL | CNNTransferLearning |
| | | | model.epoch_5- | | | |
| | | | step_19885.pkl | | | |
| 56 | 0.05265957 | L1KLmixed | | | BassetBranched | CNNBasicTraining |
| 57 | | L1KLmixed | gs://syrgoth/my- | 54 | BassetVL | CNNTransferLearning |
| | | | model.epoch_5- | | | |
| | | | step_19885.pkl | | | |
| 58 | 0.4541626408139299 | L1KLmixed | gs://syrgoth/my- | 30 | BassetBranched | CNNTransferLearning |
| | | | model.epoch_5- | | | |
| | | | step_19885.pkl | | | |
| 59 | 0.4259850687744869 | L1KLmixed | gs://syrgoth/my- | 29 | BassetBranched | CNNTransferLearning |
| | | | model.epoch_5- | | | |
| | | | step_19885.pkl | | | |
| 60 | 0.4442052351579614 | L1KLmixed | gs://syrgoth/my- | 26 | BassetBranched | CNNTransferLearning |
| | | | model.epoch_5- | | | |
| | | | step_19885.pkl | | | |
| 61 | | L1KLmixed | gs://syrgoth/my- | 57 | BassetVL | CNNTransferLearning |
| | | | model.epoch_5- | | | |
| | | | step_19885.pkl | | | |
| 62 | | MSEKLmixed | gs://syrgoth/my- | 30 | BassetVL | CNNTransferLearning |
| | | | model.epoch_5- | | | |
| | | | step_19885.pkl | | | |
| 63 | 0.4553591979886291 | L1KLmixed | gs://syrgoth/my- | 28 | BassetBranched | CNNTransferLearning |
| | | | model.epoch_5- | | | |
| | | | step_19885.pkl | | | |
| 64 | | L1KLmixed | gs://syrgoth/my- | 51 | BassetVL | CNNTransferLearning |
| | | | model.epoch_5- | | | |
| | | | step_19885.pkl | | | |
| 65 | | L1KLmixed | gs://syrgoth/my- | 38 | BassetVL | CNNTransferLearning |
| | | | model.epoch_5- | | | |
| | | | step_19885.pkl | | | |
| 66 | 0.3518673376078081 | L1KLmixed | gs://syrgoth/my- | 40 | BassetBranched | CNNTransferLearning |
| | | | model.epoch_5- | | | |
| | | | step_19885.pkl | | | |
| 67 | 0.3594715874412376 | L1KLmixed | gs://syrgoth/my- | 22 | BassetBranched | CNNTransferLearning |
| | | | model.epoch_5- | | | |
| | | | step_19885.pkl | | | |
| 68 | | L1KLmixed | gs://syrgoth/my- | 48 | BassetVL | CNNTransferLearning |
| | | | model.epoch_5- | | | |
| | | | step_19885.pkl | | | |
| 69 | 0.4464619257915677 | L1KLmixed | gs://syrgoth/my- | 20 | BassetBranched | CNNTransferLearning |
| | | | model.epoch_5- | | | |
| | | | step_19885.pkl | | | |
| 70 | | L1KLmixed | gs://syrgoth/my- | 44 | BassetVL | CNNTransferLearning |
| | | | model.epoch_5- | | | |
| | | | step_19885.pkl | | | |
| 71 | 0.4424991452791332 | L1KLmixed | gs://syrgoth/my- | 27 | BassetBranched | CNNTransferLearning |
| | | | model.epoch_5- | | | |
| | | | step_19885.pkl | | | |
| 72 | | L1KLmixed | | | BassetVL | CNNBasicTraining |
| 73 | 0.30977916 | L1KLmixed | | | BassetBranched | CNNBasicTraining |
| 74 | 0.6184177844133639 | MSEKLmixed | | | BassetBranched | CNNBasicTraining |
| 75 | | L1KLmixed | gs://syrgoth/my- | 46 | BassetVL | CNNTransferLearning |
| | | | model.epoch_5- | | | |
| | | | step_19885.pkl | | | |
| 76 | 0.3601840061280594 | L1KLmixed | gs://syrgoth/my- | 19 | BassetBranched | CNNTransferLearning |
| | | | model.epoch_5- | | | |
| | | | step_19885.pkl | | | |
| 77 | | L1KLmixed | | | BassetVL | CNNBasicTraining |
| 78 | 0.18495034 | L1KLmixed | gs://syrgoth/my- | 24 | BassetBranched | CNNTransferLearning |
| | | | model.epoch_5- | | | |
| | | | step_19885.pkl | | | |
| 79 | | L1KLmixed | | | BassetVL | CNNBasicTraining |
| 80 | 0.41428374 | L1KLmixed | gs://syrgoth/my- | 16 | BassetBranched | CNNTransferLearning |
| | | | model.epoch_5- | | | |
| | | | step_19885.pkl | | | |
| 81 | 0.4254372055662117 | L1KLmixed | gs://syrgoth/my- | 25 | BassetBranched | CNNTransferLearning |
| | | | model.epoch_5- | | | |
| | | | step_19885.pkl | | | |
| 82 | 0.4934913477819971 | L1KLmixed | gs://syrgoth/my- | 22 | BassetBranched | CNNTransferLearning |
| | | | model.epoch_5- | | | |
| | | | step_19885.pkl | | | |
| 83 | 0.4580092768093109 | L1KLmixed | gs://syrgoth/my- | 27 | BassetBranched | CNNTransferLearning |
| | | | model.epoch_5- | | | |
| | | | step_19885.pkl | | | |
| 84 | | L1KLmixed | | | BassetVL | CNNBasicTraining |
| 85 | 0.4525780232137547 | L1KLmixed | gs://syrgoth/my- | 32 | BassetBranched | CNNTransferLearning |
| | | | model.epoch_5- | | | |
| | | | step_19885.pkl | | | |
| 86 | | L1KLmixed | gs://syrgoth/my- | 52 | BassetVL | CNNTransferLearning |
| | | | model.epoch_5- | | | |
| | | | step_19885.pkl | | | |
| 87 | | L1KLmixed | gs://syrgoth/my- | 47 | BassetVL | CNNTransferLearning |
| | | | model.epoch_5- | | | |
| | | | step_19885.pkl | | | |
| 88 | | L1KLmixed | gs://syrgoth/my- | 45 | BassetVL | CNNTransferLearning |
| | | | model.epoch_5- | | | |
| | | | step_19885.pkl | | | |
| 89 | | L1KLmixed | gs://syrgoth/my- | 49 | BassetVL | CNNTransferLearning |
| | | | model.epoch_5- | | | |
| | | | step_19885.pkl | | | |
| 90 | 0.3999999999999999 | MSEKLmixed | gs://syrgoth/my- | 30 | BassetBranched | CNNTransferLearning |
| | | | model.epoch_5- | | | |
| | | | step_19885.pkl | | | |
| 91 | | L1KLmixed | | | BassetVL | CNNBasicTraining |
| 92 | | L1KLmixed | gs://syrgoth/my- | 53 | BassetVL | CNNTransferLearning |
| | | | model.epoch_5- | | | |
| | | | step_19885.pkl | | | |
| 93 | 0.5380587237823136 | L1KLmixed | gs://syrgoth/my- | 26 | BassetBranched | CNNTransferLearning |
| | | | model.epoch_5- | | | |
| | | | step_19885.pkl | | | |
| 94 | 0.3432200456417136 | L1KLmixed | gs://syrgoth/my- | 16 | BassetBranched | CNNTransferLearning |
| | | | model.epoch_5- | | | |
| | | | step_19885.pkl | | | |
| 95 | 0.5346314020541238 | L1KLmixed | gs://syrgoth/my- | 25 | BassetBranched | CNNTransferLearning |
| | | | model.epoch_5- | | | |
| | | | step_19885.pkl | | | |
| 96 | 0.1205532523363524 | MSEKLmixed | | | BassetBranched | CNNBasicTraining |
| 97 | 0.4502071598140416 | L1KLmixed | gs://syrgoth/my- | 32 | BassetBranched | CNNTransferLearning |
| | | | model.epoch_5- | | | |
| | | | step_19885.pkl | | | |
| 98 | | L1KLmixed | gs://syrgoth/my- | 48 | BassetVL | CNNTransferLearning |
| | | | model.epoch_5- | | | |
| | | | step_19885.pkl | | | |
| 99 | 0.4476157786764963 | L1KLmixed | gs://syrgoth/my- | 0 | BassetBranched | CNNTransferLearning |
| | | | model.epoch_5- | | | |
| | | | step_19885.pkl | | | |
| 100 | | L1KLmixed | gs://syrgoth/my- | 37 | BassetVL | CNNTransferLearning |
| | | | model.epoch_5- | | | |
| | | | step_19885.pkl | | | |
| 101 | | L1KLmixed | gs://syrgoth/my- | 52 | BassetVL | CNNTransferLearning |
| | | | model.epoch_5- | | | |
| | | | step_19885.pkl | | | |
| 102 | 0.58705267 | L1KLmixed | gs://syrgoth/my- | 20 | BassetBranched | CNNTransferLearning |
| | | | model.epoch_5- | | | |
| | | | step_19885.pkl | | | |
| 103 | 0.4718703264199602 | L1KLmixed | gs://syrgoth/my- | 20 | BassetBranched | CNNTransferLearning |
| | | | model.epoch_5- | | | |
| | | | step_19885.pkl | | | |
| 104 | | L1KLmixed | gs://syrgoth/my- | 45 | BassetVL | CNNTransferLearning |
| | | | model.epoch_5- | | | |
| | | | step_19885.pkl | | | |
| 105 | | L1KLmixed | gs://syrgoth/my- | 37 | BassetVL | CNNTransferLearning |
| | | | model.epoch_5- | | | |
| | | | step_19885.pkl | | | |
| 106 | 0.3994299004050705 | L1KLmixed | gs://syrgoth/my- | 4 | BassetBranched | CNNTransferLearning |
| | | | model.epoch_5- | | | |
| | | | step_19885.pkl | | | |
| 107 | 0.4554369636678926 | L1KLmixed | gs://syrgoth/my- | 28 | BassetBranched | CNNTransferLearning |
| | | | model.epoch_5- | | | |
| | | | step_19885.pkl | | | |
| 108 | | L1KLmixed | gs://syrgoth/my- | 56 | BassetVL | CNNTransferLearning |
| | | | model.epoch_5- | | | |
| | | | step_19885.pkl | | | |
| 109 | 0.4947616728548538 | L1KLmixed | gs://syrgoth/my- | 22 | BassetBranched | CNNTransferLearning |
| | | | model.epoch_5- | | | |
| | | | step_19885.pkl | | | |
| 110 | | MSEKLmixed | gs://syrgoth/my- | 0 | BassetVL | CNNTransferLearning |
| | | | model.epoch_5- | | | |
| | | | step_19885.pkl | | | |
| 111 | 0.4127297205736704 | L1KLmixed | gs://syrgoth/my- | 23 | BassetBranched | CNNTransferLearning |
| | | | model.epoch_5- | | | |
| | | | step_19885.pkl | | | |
| 112 | | L1KLmixed | gs://syrgoth/my- | 50 | BassetVL | CNNTransferLearning |
| | | | model.epoch_5- | | | |
| | | | step_19885.pkl | | | |
| 113 | 0.3602965584742966 | L1KLmixed | gs://syrgoth/my- | 20 | BassetBranched | CNNTransferLearning |
| | | | model.epoch_5- | | | |
| | | | step_19885.pkl | | | |
| 114 | 0.4768886646608617 | L1KLmixed | gs://syrgoth/my- | 32 | BassetBranched | CNNTransferLearning |
| | | | model.epoch_5- | | | |
| | | | step_19885.pkl | | | |
| 115 | | L1KLmixed | gs://syrgoth/my- | 48 | BassetVL | CNNTransferLearning |
| | | | model.epoch_5- | | | |
| | | | step_19885.pkl | | | |
| 116 | | L1KLmixed | gs://syrgoth/my- | 55 | BassetVL | CNNTransferLearning |
| | | | model.epoch_5- | | | |
| | | | step_19885.pkl | | | |
| 117 | 0.2851460861435649 | L1KLmixed | gs://syrgoth/my- | 45 | BassetBranched | CNNTransferLearning |
| | | | model.epoch_5- | | | |
| | | | step_19885.pkl | | | |
| 118 | | L1KLmixed | gs://syrgoth/my- | 34 | BassetVL | CNNTransferLearning |
| | | | model.epoch_5- | | | |
| | | | step_19885.pkl | | | |
| 119 | 0.5265813839011152 | L1KLmixed | gs://syrgoth/my- | 20 | BassetBranched | CNNTransferLearning |
| | | | model.epoch_5- | | | |
| | | | step_19885.pkl | | | |
| 120 | 0.53491241 | MSEKLmixed | gs://syrgoth/my- | 24 | BassetBranched | CNNTransferLearning |
| | | | model.epoch_5- | | | |
| | | | step_19885.pkl | | | |
| 121 | 0.4481044000075117 | L1KLmixed | gs://syrgoth/my- | 49 | BassetBranched | CNNTransferLearning |
| | | | model.epoch_5- | | | |
| | | | step_19885.pkl | | | |
| 122 | 0.2832605640064339 | L1KLmixed | gs://syrgoth/my- | 34 | BassetBranched | CNNTransferLearning |
| | | | model.epoch_5- | | | |
| | | | step_19885.pkl | | | |
| 123 | 0.5278840403687162 | L1KLmixed | gs://syrgoth/my- | 45 | BassetBranched | CNNTransferLearning |
| | | | model.epoch_5- | | | |
| | | | step_19885.pkl | | | |
| 124 | 0.45093202 | L1KLmixed | gs://syrgoth/my- | 26 | BassetBranched | CNNTransferLearning |
| | | | model.epoch_5- | | | |
| | | | step_19885.pkl | | | |
| 125 | | L1KLmixed | gs://syrgoth/my- | 24 | BassetVL | CNNTransferLearning |
| | | | model.epoch_5- | | | |
| | | | step_19885.pkl | | | |
| 126 | | L1KLmixed | gs://syrgoth/my- | 42 | BassetVL | CNNTransferLearning |
| | | | model.epoch_5- | | | |
| | | | step_19885.pkl | | | |
| 127 | | L1KLmixed | gs://syrgoth/my- | 60 | BassetVL | CNNTransferLearning |
| | | | model.epoch_5- | | | |
| | | | step_19885.pkl | | | |
| 128 | 0.3699153708453486 | L1KLmixed | gs://syrgoth/my- | 32 | BassetBranched | CNNTransferLearning |
| | | | model.epoch_5- | | | |
| | | | step_19885.pkl | | | |
| 129 | 0.5462974616103523 | L1KLmixed | gs://syrgoth/my- | 20 | BassetBranched | CNNTransferLearning |
| | | | model.epoch_5- | | | |
| | | | step_19885.pkl | | | |
| 130 | 0.3756893340651077 | MSEKLmixed | gs://syrgoth/my- | 14 | BassetBranched | CNNTransferLearning |
| | | | model.epoch_5- | | | |
| | | | step_19885.pkl | | | |
| 131 | 0.3380185194693155 | L1KLmixed | gs://syrgoth/my- | 42 | BassetBranched | CNNTransferLearning |
| | | | model.epoch_5- | | | |
| | | | step_19885.pkl | | | |
| 132 | 0.3670477190614801 | L1KLmixed | gs://syrgoth/my- | 16 | BassetBranched | CNNTransferLearning |
| | | | model.epoch_5- | | | |
| | | | step_19885.pkl | | | |
| 133 | | L1KLmixed | gs://syrgoth/my- | 25 | BassetVL | CNNTransferLearning |
| | | | model.epoch_5- | | | |
| | | | step_19885.pkl | | | |
| 134 | 0.4534637557799335 | L1KLmixed | gs://syrgoth/my- | 29 | BassetBranched | CNNTransferLearning |
| | | | model.epoch_5- | | | |
| | | | step_19885.pkl | | | |
| 135 | 0.0788459 | L1KLmixed | | | BassetBranched | CNNBasicTraining |
| 136 | 0.2788282426750011 | L1KLmixed | gs://syrgoth/my- | 51 | BassetBranched | CNNTransferLearning |
| | | | model.epoch_5- | | | |
| | | | step_19885.pkl | | | |
| 137 | 0.1256315633171712 | L1KLmixed | | | BassetBranched | CNNBasicTraining |
| 138 | 0.1474199621874418 | L1KLmixed | | | BassetBranched | CNNBasicTraining |
| 139 | 0.05290452 | L1KLmixed | | | BassetBranched | CNNBasicTraining |
| 140 | 0.09221454 | L1KLmixed | | | BassetBranched | CNNBasicTraining |
| 141 | | L1KLmixed | gs://syrgoth/my- | 48 | BassetVL | CNNTransferLearning |
| | | | model.epoch_5- | | | |
| | | | step_19885.pkl | | | |
| 142 | 0.48828775 | L1KLmixed | gs://syrgoth/my- | 33 | BassetBranched | CNNTransferLearning |
| | | | model.epoch_5- | | | |
| | | | step_19885.pkl | | | |
| 143 | 0.0509318 | L1KLmixed | | | BassetBranched | CNNBasicTraining |
| 144 | | L1KLmixed | gs://syrgoth/my- | 52 | BassetVL | CNNTransferLearning |
| | | | model.epoch_5- | | | |
| | | | step_19885.pkl | | | |
| 145 | | MSEKLmixed | gs://syrgoth/my- | 54 | BassetVL | CNNTransferLearning |
| | | | model.epoch_5- | | | |
| | | | step_19885.pkl | | | |
| 146 | | MSEKLmixed | | | BassetVL | CNNBasicTraining |
| 147 | | L1KLmixed | gs://syrgoth/my- | 31 | BassetVL | CNNTransferLearning |
| | | | model.epoch_5- | | | |
| | | | step_19885.pkl | | | |
| 148 | 0.05 | L1KLmixed | | | BassetBranched | CNNBasicTraining |
| 149 | | L1KLmixed | gs://syrgoth/my- | 42 | BassetVL | CNNTransferLearning |
| | | | model.epoch_5- | | | |
| | | | step_19885.pkl | | | |
| 150 | 0.3276404 | L1KLmixed | gs://syrgoth/my- | 40 | BassetBranched | CNNTransferLearning |
| | | | model.epoch_5- | | | |
| | | | step_19885.pkl | | | |
| 151 | 0.4336905413867709 | L1KLmixed | gs://syrgoth/my- | 29 | BassetBranched | CNNTransferLearning |
| | | | model.epoch_5- | | | |
| | | | step_19885.pkl | | | |
| 152 | | L1KLmixed | | | BassetVL | CNNBasicTraining |
| 153 | 0.4399575579906737 | L1KLmixed | gs://syrgoth/my- | 32 | BassetBranched | CNNTransferLearning |
| | | | model.epoch_5- | | | |
| | | | step_19885.pkl | | | |
| 154 | | L1KLmixed | | | BassetVL | CNNBasicTraining |
| 155 | | L1KLmixed | | | BassetVL | CNNBasicTraining |
| 156 | | L1KLmixed | gs://syrgoth/my- | 60 | BassetVL | CNNTransferLearning |
| | | | model.epoch_5- | | | |
| | | | step_19885.pkl | | | |
| 157 | | L1KLmixed | gs://syrgoth/my- | 41 | BassetVL | CNNTransferLearning |
| | | | model.epoch_5- | | | |
| | | | step_19885.pkl | | | |
| 158 | 0.3415005386955621 | L1KLmixed | gs://syrgoth/my- | 38 | BassetBranched | CNNTransferLearning |
| | | | model.epoch_5- | | | |
| | | | step_19885.pkl | | | |
| 159 | 0.4900891558004834 | L1KLmixed | gs://syrgoth/my- | 45 | BassetBranched | CNNTransferLearning |
| | | | model.epoch_5- | | | |
| | | | step_19885.pkl | | | |
| 160 | 0.05332208 | L1KLmixed | | | BassetBranched | CNNBasicTraining |
| 161 | 0.12871921 | L1KLmixed | | | BassetBranched | CNNBasicTraining |
| 162 | | L1KLmixed | | | BassetVL | CNNBasicTraining |
| 163 | | MSEKLmixed | gs://syrgoth/my- | 44 | BassetVL | CNNTransferLearning |
| | | | model.epoch_5- | | | |
| | | | step_19885.pkl | | | |
| 164 | | L1KLmixed | gs://syrgoth/my- | 46 | BassetVL | CNNTransferLearning |
| | | | model.epoch_5- | | | |
| | | | step_19885.pkl | | | |
| 165 | | L1KLmixed | gs://syrgoth/my- | 39 | BassetVL | CNNTransferLearning |
| | | | model.epoch_5- | | | |
| | | | step_19885.pkl | | | |
| 166 | | L1KLmixed | | | BassetVL | CNNBasicTraining |
| 167 | 0.05128954 | L1KLmixed | | | BassetBranched | CNNBasicTraining |
| 168 | 0.05272222 | L1KLmixed | | | BassetBranched | CNNBasicTraining |
| 169 | | L1KLmixed | | | BassetVL | CNNBasicTraining |
| 170 | 0.1000486563445668 | L1KLmixed | | | BassetBranched | CNNBasicTraining |
| 171 | 0.05033796 | L1KLmixed | | | BassetBranched | CNNBasicTraining |
| 172 | 0.05284977 | L1KLmixed | | | BassetBranched | CNNBasicTraining |
| 173 | 0.3951046414834008 | L1KLmixed | gs://syrgoth/my- | 31 | BassetBranched | CNNTransferLearning |
| | | | model.epoch_5- | | | |
| | | | step_19885.pkl | | | |
| 174 | 0.4873144760691454 | L1KLmixed | gs://syrgoth/my- | 31 | BassetBranched | CNNTransferLearning |
| | | | model.epoch_5- | | | |
| | | | step_19885.pkl | | | |
| 175 | | L1KLmixed | gs://syrgoth/my- | 47 | BassetVL | CNNTransferLearning |
| | | | model.epoch_5- | | | |
| | | | step_19885.pkl | | | |
| 176 | 0.47965351 | L1KLmixed | gs://syrgoth/my- | 26 | BassetBranched | CNNTransferLearning |
| | | | model.epoch_5- | | | |
| | | | step_19885.pkl | | | |
| 177 | | L1KLmixed | gs://syrgoth/my- | 36 | BassetVL | CNNTransferLearning |
| | | | model.epoch_5- | | | |
| | | | step_19885.pkl | | | |
| 178 | 0.3408185649334015 | L1KLmixed | gs://syrgoth/my- | 38 | BassetBranched | CNNTransferLearning |
| | | | model.epoch_5- | | | |
| | | | step_19885.pkl | | | |
| 179 | 0.3247539257693671 | L1KLmixed | gs://syrgoth/my- | 37 | BassetBranched | CNNTransferLearning |
| | | | model.epoch_5- | | | |
| | | | step_19885.pkl | | | |
| 180 | 0.3585748458287149 | L1KLmixed | gs://syrgoth/my- | 22 | BassetBranched | CNNTransferLearning |
| | | | model.epoch_5- | | | |
| | | | step_19885.pkl | | | |
| 181 | 0.4349252613471183 | L1KLmixed | gs://syrgoth/my- | 39 | BassetBranched | CNNTransferLearning |
| | | | model.epoch_5- | | | |
| | | | step_19885.pkl | | | |
| 182 | | L1KLmixed | | | BassetVL | CNNBasicTraining |
| 183 | | L1KLmixed | gs://syrgoth/my- | 46 | BassetVL | CNNTransferLearning |
| | | | model.epoch_5- | | | |
| | | | step_19885.pkl | | | |
| 184 | 0.3907929225916775 | L1KLmixed | gs://syrgoth/my- | 24 | BassetBranched | CNNTransferLearning |
| | | | model.epoch_5- | | | |
| | | | step_19885.pkl | | | |
| 185 | 0.75 | L1KLmixed | gs://syrgoth/my- | 34 | BassetBranched | CNNTransferLearning |
| | | | model.epoch_5- | | | |
| | | | step_19885.pkl | | | |
| 186 | | L1KLmixed | gs://syrgoth/my- | 45 | BassetVL | CNNTransferLearning |
| | | | model.epoch_5- | | | |
| | | | step_19885.pkl | | | |
| 187 | 0.2647668779974836 | L1KLmixed | | | BassetBranched | CNNBasicTraining |
| 188 | | L1KLmixed | gs://syrgoth/my- | 54 | BassetVL | CNNTransferLearning |
| | | | model.epoch_5- | | | |
| | | | step_19885.pkl | | | |
| 189 | 0.38812137 | MSEKLmixed | gs://syrgoth/my- | 29 | BassetBranched | CNNTransferLearning |
| | | | model.epoch_5- | | | |
| | | | step_19885.pkl | | | |
| 190 | 0.2296230903094931 | L1KLmixed | | | BassetBranched | CNNBasicTraining |
| 191 | | L1KLmixed | | | BassetVL | CNNBasicTraining |
| 192 | 0.4631163338905634 | L1KLmixed | gs://syrgoth/my- | 38 | BassetBranched | CNNTransferLearning |
| | | | model.epoch_5- | | | |
| | | | step_19885.pkl | | | |
| 193 | | L1KLmixed | gs://syrgoth/my- | 33 | BassetVL | CNNTransferLearning |
| | | | model.epoch_5- | | | |
| | | | step_19885.pkl | | | |
| 194 | | L1KLmixed | gs://syrgoth/my- | 22 | BassetVL | CNNTransferLearning |
| | | | model.epoch_5- | | | |
| | | | step_19885.pkl | | | |
| 195 | | L1KLmixed | gs://syrgoth/my- | 44 | BassetVL | CNNTransferLearning |
| | | | model.epoch_5- | | | |
| | | | step_19885.pkl | | | |
| 196 | | L1KLmixed | gs://syrgoth/my- | 50 | BassetVL | CNNTransferLearning |
| | | | model.epoch_5- | | | |
| | | | step_19885.pkl | | | |
| 197 | | L1KLmixed | gs://syrgoth/my- | 60 | BassetVL | CNNTransferLearning |
| | | | model.epoch_5- | | | |
| | | | step_19885.pkl | | | |
| 198 | 0.2262653416385505 | L1KLmixed | gs://syrgoth/my- | 0 | BassetBranched | CNNTransferLearning |
| | | | model.epoch_5- | | | |
| | | | step_19885.pkl | | | |
| 199 | 0.3265599351677913 | L1KLmixed | gs://syrgoth/my- | 38 | BassetBranched | CNNTransferLearning |
| | | | model.epoch_5- | | | |
| | | | step_19885.pkl | | | |
| 200 | 0.3934476905632549 | L1KLmixed | gs://syrgoth/my- | 39 | BassetBranched | CNNTransferLearning |
| | | | model.epoch_5- | | | |
| | | | step_19885.pkl | | | |
| 201 | 0.3458614673609552 | L1KLmixed | gs://syrgoth/my- | 39 | BassetBranched | CNNTransferLearning |
| | | | model.epoch_5- | | | |
| | | | step_19885.pkl | | | |
| 202 | | L1KLmixed | | | BassetVL | CNNBasicTraining |
| 203 | | L1KLmixed | gs://syrgoth/my- | 39 | BassetVL | CNNTransferLearning |
| | | | model.epoch_5- | | | |
| | | | step_19885.pkl | | | |
| 204 | | L1KLmixed | | | BassetVL | CNNBasicTraining |
| 205 | 0.4657199193591799 | L1KLmixed | gs://syrgoth/my- | 28 | BassetBranched | CNNTransferLearning |
| | | | model.epoch_5- | | | |
| | | | step_19885.pkl | | | |
| 206 | | L1KLmixed | gs://syrgoth/my- | 38 | BassetVL | CNNTransferLearning |
| | | | model.epoch_5- | | | |
| | | | step_19885.pkl | | | |
| 207 | | MSEKLmixed | | | BassetVL | CNNBasicTraining |
| 208 | 0.3644342742785668 | L1KLmixed | gs://syrgoth/my- | 40 | BassetBranched | CNNTransferLearning |
| | | | model.epoch_5- | | | |
| | | | step_19885.pkl | | | |
| 209 | | L1KLmixed | gs://syrgoth/my- | 42 | BassetVL | CNNTransferLearning |
| | | | model.epoch_5- | | | |
| | | | step_19885.pkl | | | |
| 210 | 0.1498661562249081 | L1KLmixed | | | BassetBranched | CNNBasicTraining |
| 211 | 0.4704425378036096 | L1KLmixed | gs://syrgoth/my- | 27 | BassetBranched | CNNTransferLearning |
| | | | model.epoch_5- | | | |
| | | | step_19885.pkl | | | |
| 212 | 0.5786708964865738 | L1KLmixed | gs://syrgoth/my- | 33 | BassetBranched | CNNTransferLearning |
| | | | model.epoch_5- | | | |
| | | | step_19885.pkl | | | |
| 213 | 0.2425958574808762 | L1KLmixed | | | BassetBranched | CNNBasicTraining |
| 214 | | L1KLmixed | gs://syrgoth/my- | 50 | BassetVL | CNNTransferLearning |
| | | | model.epoch_5- | | | |
| | | | step_19885.pkl | | | |
| 215 | 0.4064863906426788 | MSEKLmixed | gs://syrgoth/my- | 32 | BassetBranched | CNNTransferLearning |
| | | | model.epoch_5- | | | |
| | | | step_19885.pkl | | | |
| 216 | | L1KLmixed | gs://syrgoth/my- | 14 | BassetVL | CNNTransferLearning |
| | | | model.epoch_5- | | | |
| | | | step_19885.pkl | | | |
| 217 | 0.4279687249212062 | L1KLmixed | gs://syrgoth/my- | 22 | BassetBranched | CNNTransferLearning |
| | | | model.epoch_5- | | | |
| | | | step_19885.pkl | | | |
| 218 | | L1KLmixed | gs://syrgoth/my- | 47 | BassetVL | CNNTransferLearning |
| | | | model.epoch_5- | | | |
| | | | step_19885.pkl | | | |
| 219 | | L1KLmixed | gs://syrgoth/my- | 45 | BassetVL | CNNTransferLearning |
| | | | model.epoch_5- | | | |
| | | | step_19885.pkl | | | |
| 220 | | L1KLmixed | gs://syrgoth/my- | 49 | BassetVL | CNNTransferLearning |
| | | | model.epoch_5- | | | |
| | | | step_19885.pkl | | | |
| 221 | 0.3216323362044054 | L1KLmixed | gs://syrgoth/my- | 13 | BassetBranched | CNNTransferLearning |
| | | | model.epoch_5- | | | |
| | | | step_19885.pkl | | | |
| 222 | | L1KLmixed | gs://syrgoth/my- | 46 | BassetVL | CNNTransferLearning |
| | | | model.epoch_5- | | | |
| | | | step_19885.pkl | | | |
| 223 | 0.3514613712542899 | MSEKLmixed | gs://syrgoth/my- | 55 | BassetBranched | CNNTransferLearning |
| | | | model.epoch_5- | | | |
| | | | step_19885.pkl | | | |
| 224 | | L1KLmixed | gs://syrgoth/my- | 41 | BassetVL | CNNTransferLearning |
| | | | model.epoch_5- | | | |
| | | | step_19885.pkl | | | |
| 225 | 0.2396904077593232 | L1KLmixed | | | BassetBranched | CNNBasicTraining |
| 226 | | MSEKLmixed | | | BassetVL | CNNBasicTraining |
| 227 | 0.05 | MSEKLmixed | | | BassetBranched | CNNBasicTraining |
| 228 | 0.4574166323441865 | L1KLmixed | gs://syrgoth/my- | 33 | BassetBranched | CNNTransferLearning |
| | | | model.epoch_5- | | | |
| | | | step_19885.pkl | | | |
| 229 | 0.05780124 | MSEKLmixed | | | BassetBranched | CNNBasicTraining |
| 230 | 0.3302541730698451 | MSEKLmixed | | | BassetBranched | CNNBasicTraining |
| 231 | 0.4134181609028136 | L1KLmixed | gs://syrgoth/my- | 27 | BassetBranched | CNNTransferLearning |
| | | | model.epoch_5- | | | |
| | | | step_19885.pkl | | | |
| 232 | 0.4748724430400129 | L1KLmixed | gs://syrgoth/my- | 24 | BassetBranched | CNNTransferLearning |
| | | | model.epoch_5- | | | |
| | | | step_19885.pkl | | | |
| 233 | 0.4177989004155676 | L1KLmixed | gs://syrgoth/my- | 30 | BassetBranched | CNNTransferLearning |
| | | | model.epoch_5- | | | |
| | | | step_19885.pkl | | | |
| 234 | 0.05128666 | MSEKLmixed | | | BassetBranched | CNNBasicTraining |
| 235 | 0.4459855764079179 | L1KLmixed | gs://syrgoth/my- | 27 | BassetBranched | CNNTransferLearning |
| | | | model.epoch_5- | | | |
| | | | step_19885.pkl | | | |
| 236 | 0.4836818683159359 | L1KLmixed | gs://syrgoth/my- | 20 | BassetBranched | CNNTransferLearning |
| | | | model.epoch_5- | | | |
| | | | step_19885.pkl | | | |
| 237 | | L1KLmixed | gs://syrgoth/my- | 59 | BassetVL | CNNTransferLearning |
| | | | model.epoch_5- | | | |
| | | | step_19885.pkl | | | |
| 238 | | L1KLmixed | gs://syrgoth/my- | 60 | BassetVL | CNNTransferLearning |
| | | | model.epoch_5- | | | |
| | | | step_19885.pkl | | | |
| 239 | 0.4765367369713966 | L1KLmixed | gs://syrgoth/my- | 18 | BassetBranched | CNNTransferLearning |
| | | | model.epoch_5- | | | |
| | | | step_19885.pkl | | | |
| 240 | 0.3025085660548822 | L1KLmixed | gs://syrgoth/my- | 38 | BassetBranched | CNNTransferLearning |
| | | | model.epoch_5- | | | |
| | | | step_19885.pkl | | | |
| 241 | 0.3115316151928443 | L1KLmixed | | | BassetBranched | CNNBasicTraining |
| 242 | 0.4265881931583789 | L1KLmixed | gs://syrgoth/my- | 22 | BassetBranched | CNNTransferLearning |
| | | | model.epoch_5- | | | |
| | | | step_19885.pkl | | | |
| 243 | 0.4883570982424499 | L1KLmixed | gs://syrgoth/my- | 29 | BassetBranched | CNNTransferLearning |
| | | | model.epoch_5- | | | |
| | | | step_19885.pkl | | | |
| 244 | 0.05563484 | MSEKLmixed | | | BassetBranched | CNNBasicTraining |
| 245 | 0.4640533621132366 | MSEKLmixed | | | BassetBranched | CNNBasicTraining |
| 246 | 0.2805868132033031 | L1KLmixed | gs://syrgoth/my- | 37 | BassetBranched | CNNTransferLearning |
| | | | model.epoch_5- | | | |
| | | | step_19885.pkl | | | |
| 247 | 0.43742695 | L1KLmixed | | | BassetBranched | CNNBasicTraining |
| 248 | 0.4594863828090411 | L1KLmixed | gs://syrgoth/my-28 | | BassetBranched | CNNTransferLearning |
| | | | model.epoch_5- | | | |
| | | | step_19885.pkl | | | |
| 249 | 0.1778713677330271 | MSEKLmixed | | | BassetBranched | CNNBasicTraining |
| 250 | | L1KLmixed | | | BassetVL | CNNBasicTraining |
| 251 | 0.4215479128184451 | L1KLmixed | gs://syrgoth/my-36 | | BassetBranched | CNNTransferLearning |
| | | | model.epoch_5- | | | |
| | | | step_19885.pkl | | | |
| 252 | | MSEKLmixed | | | BassetVL | CNNBasicTraining |
| 253 | | L1KLmixed | gs://syrgoth/my- | 46 | BassetVL | CNNTransferLearning |
| | | | model.epoch_5- | | | |
| | | | step_19885.pkl | | | |
| 254 | | L1KLmixed | | | BassetVL | CNNBasicTraining |
| 255 | | MSEKLmixed | | | BassetVL | CNNBasicTraining |
| 256 | 0.4704449987197819 | L1KLmixed | gs://syrgoth/my- | 33 | BassetBranched | CNNTransferLearning |
| | | | model.epoch_5- | | | |
| | | | step_19885.pkl | | | |
| 257 | 0.05116522 | L1KLmixed | | | BassetBranched | CNNBasicTraining |
| 258 | | L1KLmixed | | | BassetVL | CNNBasicTraining |
| 259 | | L1KLmixed | gs://syrgoth/my- | 30 | BassetVL | CNNTransferLearning |
| | | | model.epoch_5- | | | |
| | | | step_19885.pkl | | | |
| 260 | | L1KLmixed | gs://syrgoth/my- | 60 | BassetVL | CNNTransferLearning |
| | | | model.epoch_5- | | | |
| | | | step_19885.pkl | | | |
| 261 | | L1KLmixed | | | BassetVL | CNNBasicTraining |
| 262 | | L1KLmixed | gs://syrgoth/my- | 50 | BassetVL | CNNTransferLearning |
| | | | model.epoch_5- | | | |
| | | | step_19885.pkl | | | |
| 263 | | L1KLmixed | gs://syrgoth/my- | 49 | BassetVL | CNNTransferLearning |
| | | | model.epoch_5- | | | |
| | | | step_19885.pkl | | | |
| 264 | 0.08272623 | L1KLmixed | | | BassetBranched | CNNBasicTraining |
| 265 | 0.07957457 | MSEKLmixed | | | BassetBranched | CNNBasicTraining |
| 266 | | L1KLmixed | | | BassetVL | CNNBasicTraining |
| 267 | 0.4547848872397854 | L1KLmixed | gs://syrgoth/my-32 | | BassetBranched | CNNTransferLearning |
| | | | model.epoch_5- | | | |
| | | | step_19885.pkl | | | |
| 268 | | L1KLmixed | gs://syrgoth/my-54 | | BassetVL | CNNTransferLearning |
| | | | model.epoch_5- | | | |
| | | | step_19885.pkl | | | |
| 269 | | L1KLmixed | | | BassetVL | CNNBasicTraining |
| 270 | | L1KLmixed | | | BassetVL | CNNBasicTraining |
| 271 | 0.1880010394762066 | MSEKLmixed | gs://syrgoth/my- | 41 | BassetBranched | CNNTransferLearning |
| | | | model.epoch_5- | | | |
| | | | step_19885.pkl | | | |
| 272 | 0.4349560958708773 | L1KLmixed | gs://syrgoth/my- | 30 | BassetBranched | CNNTransferLearning |
| | | | model.epoch_5- | | | |
| | | | step_19885.pkl | | | |
| 273 | | MSEKLmixed | | | BassetVL | CNNBasicTraining |
| 274 | | L1KLmixed | gs://syrgoth/my- | 51 | BassetVL | CNNTransferLearning |
| | | | model.epoch_5- | | | |
| | | | step_19885.pkl | | | |
| 275 | | L1KLmixed | gs://syrgoth/my- | 50 | BassetVL | CNNTransferLearning |
| | | | model.epoch_5- | | | |
| | | | step_19885.pkl | | | |
| 276 | 0.3924638811542661 | L1KLmixed | gs://syrgoth/my- | 36 | BassetBranched | CNNTransferLearning |
| | | | model.epoch_5- | | | |
| | | | step_19885.pkl | | | |
| 277 | 0.1478422233747757 | MSEKLmixed | | | BassetBranched | CNNBasicTraining |
| 278 | | L1KLmixed | gs://syrgoth/my- | 25 | BassetVL | CNNTransferLearning |
| | | | model.epoch_5- | | | |
| | | | step_19885.pkl | | | |
| 279 | 0.05112477 | MSEKLmixed | | | BassetBranched | CNNBasicTraining |
| 280 | | L1KLmixed | gs://syrgoth/my- | 60 | BassetVL | CNNTransferLearning |
| | | | model.epoch_5- | | | |
| | | | step_19885.pkl | | | |
| 281 | 0.5536594474963844 | L1KLmixed | gs://syrgoth/my- | 33 | BassetBranched | CNNTransferLearning |
| | | | model.epoch_5- | | | |
| | | | step_19885.pkl | | | |
| 282 | 0.3024084857535395 | L1KLmixed | gs://syrgoth/my- | 33 | BassetBranched | CNNTransferLearning |
| | | | model.epoch_5- | | | |
| | | | step_19885.pkl | | | |
| 283 | 0.2877418991807431 | L1KLmixed | gs://syrgoth/my- | 40 | BassetBranched | CNNTransferLearning |
| | | | model.epoch_5- | | | |
| | | | step_19885.pkl | | | |
| 284 | 0.39318804 | L1KLmixed | gs://syrgoth/my- | 36 | BassetBranched | CNNTransferLearning |
| | | | model.epoch_5- | | | |
| | | | step_19885.pkl | | | |
| 285 | | L1KLmixed | | | BassetVL | CNNBasicTraining |
| 286 | 0.5007415299195562 | L1KLmixed | gs://syrgoth/my- | 33 | BassetBranched | CNNTransferLearning |
| | | | model.epoch_5- | | | |
| | | | step_19885.pkl | | | |
| 287 | 0.4839308535282552 | L1KLmixed | gs://syrgoth/my- | 36 | BassetBranched | CNNTransferLearning |
| | | | model.epoch_5- | | | |
| | | | step_19885.pkl | | | |
| 288 | 0.3845682118339318 | L1KLmixed | gs://syrgoth/my- | 31 | BassetBranched | CNNTransferLearning |
| | | | model.epoch_5- | | | |
| | | | step_19885.pkl | | | |
| 289 | | L1KLmixed | gs://syrgoth/my- | 35 | BassetVL | CNNTransferLearning |
| | | | model.epoch_5- | | | |
| | | | step_19885.pkl | | | |
| 290 | | L1KLmixed | gs://syrgoth/my- | 41 | BassetVL | CNNTransferLearning |
| | | | model.epoch_5- | | | |
| | | | step_19885.pkl | | | |
| 291 | 0.05072517 | L1KLmixed | | | BassetBranched | CNNBasicTraining |
| 292 | 0.4781362149556876 | L1KLmixed | gs://syrgoth/my- | 25 | BassetBranched | CNNTransferLearning |
| | | | model.epoch_5- | | | |
| | | | step_19885.pkl | | | |
| 293 | 0.3884271975554163 | L1KLmixed | gs://syrgoth/my- | 24 | BassetBranched | CNNTransferLearning |
| | | | model.epoch_5- | | | |
| | | | step_19885.pkl | | | |
| 294 | | L1KLmixed | | | BassetVL | CNNBasicTraining |
| 295 | 0.1988402298882677 | L1KLmixed | gs://syrgoth/my- | 44 | BassetBranched | CNNTransferLearning |
| | | | model.epoch_5- | | | |
| | | | step_19885.pkl | | | |
| 296 | | L1KLmixed | gs://syrgoth/my- | 50 | BassetVL | CNNTransferLearning |
| | | | model.epoch_5- | | | |
| | | | step_19885.pkl | | | |
| 297 | 0.4746518836862812 | L1KLmixed | gs://syrgoth/my- | 33 | BassetBranched | CNNTransferLearning |
| | | | model.epoch_5- | | | |
| | | | step_19885.pkl | | | |
| 298 | | L1KLmixed | gs://syrgoth/my- | 40 | BassetVL | CNNTransferLearning |
| | | | model.epoch_5- | | | |
| | | | step_19885.pkl | | | |
| 299 | | L1KLmixed | gs://syrgoth/my- | 51 | BassetVL | CNNTransferLearning |
| | | | model.epoch_5- | | | |
| | | | step_19885.pkl | | | |
| 300 | 0.4239638999104125 | L1KLmixed | gs://syrgoth/my- | 35 | BassetBranched | CNNTransferLearning |
| | | | model.epoch_5- | | | |
| | | | step_19885.pkl | | | |
| 301 | 0.2306276262962407 | L1KLmixed | gs://syrgoth/my- | 37 | BassetBranched | CNNTransferLearning |
| | | | model.epoch_5- | | | |
| | | | step_19885.pkl | | | |
| 302 | | L1KLmixed | gs://syrgoth/my- | 47 | BassetVL | CNNTransferLearning |
| | | | model.epoch_5- | | | |
| | | | step_19885.pkl | | | |
| 303 | | L1KLmixed | gs://syrgoth/my- | 55 | BassetVL | CNNTransferLearning |
| | | | model.epoch_5- | | | |
| | | | step_19885.pkl | | | |
| 304 | 0.05 | L1KLmixed | gs://syrgoth/my- | 51 | BassetBranched | CNNTransferLearning |
| | | | model.epoch_5- | | | |
| | | | step_19885.pkl | | | |
| 305 | 0.07056246 | L1KLmixed | | | BassetBranched | CNNBasicTraining |
| 306 | | L1KLmixed | | | BassetVL | CNNBasicTraining |
| 307 | 0.3438547871803173 | L1KLmixed | gs://syrgoth/my- | 49 | BassetBranched | CNNTransferLearning |
| | | | model.epoch_5- | | | |
| | | | step_19885.pkl | | | |
| 308 | 0.05 | MSEKLmixed | | | BassetBranched | CNNBasicTraining |
| 309 | 0.4648205533036629 | L1KLmixed | gs://syrgoth/my- | 31 | BassetBranched | CNNTransferLearning |
| | | | model.epoch_5- | | | |
| | | | step_19885.pkl | | | |
| 310 | 0.3012959552827633 | L1KLmixed | gs://syrgoth/my- | 23 | BassetBranched | CNNTransferLearning |
| | | | model.epoch_5- | | | |
| | | | step_19885.pkl | | | |
| 311 | 0.3187373827537472 | MSEKLmixed | gs://syrgoth/my- | 42 | BassetBranched | CNNTransferLearning |
| | | | model.epoch_5- | | | |
| | | | step_19885.pkl | | | |
| 312 | 0.1345442562848544 | MSEKLmixed | | | BassetBranched | CNNBasicTraining |
| 313 | 0.4670807703355645 | L1KLmixed | gs://syrgoth/my- | 27 | BassetBranched | CNNTransferLearning |
| | | | model.epoch_5- | | | |
| | | | step_19885.pkl | | | |
| 314 | | L1KLmixed | | | BassetVL | CNNBasicTraining |
| 315 | 0.3713182360870709 | L1KLmixed | gs://syrgoth/my- | 36 | BassetBranched | CNNTransferLearning |
| | | | model.epoch_5- | | | |
| | | | step_19885.pkl | | | |
| 316 | | L1KLmixed | gs://syrgoth/my- | 47 | BassetVL | CNNTransferLearning |
| | | | model.epoch_5- | | | |
| | | | step_19885.pkl | | | |
| 317 | | L1KLmixed | | | BassetVL | CNNBasicTraining |
| 318 | 0.4807960794795886 | L1KLmixed | gs://syrgoth/my- | 33 | BassetBranched | CNNTransferLearning |
| | | | model.epoch_5- | | | |
| | | | step_19885.pkl | | | |
| 319 | 0.4516970544303772 | L1KLmixed | gs://syrgoth/my- | 28 | BassetBranched | CNNTransferLearning |
| | | | model.epoch_5- | | | |
| | | | step_19885.pkl | | | |
| 320 | 0.4038359239139629 | L1KLmixed | gs://syrgoth/my- | 21 | BassetBranched | CNNTransferLearning |
| | | | model.epoch_5- | | | |
| | | | step_19885.pkl | | | |
| 321 | 0.43963812 | L1KLmixed | gs://syrgoth/my- | 12 | BassetBranched | CNNTransferLearning |
| | | | model.epoch_5- | | | |
| | | | step_19885.pkl | | | |
| 322 | 0.05712258 | L1KLmixed | | | BassetBranched | CNNBasicTraining |
| 323 | | L1KLmixed | gs://syrgoth/my- | 49 | BassetVL | CNNTransferLearning |
| | | | model.epoch_5- | | | |
| | | | step_19885.pkl | | | |
| 324 | 0.4363311507859165 | L1KLmixed | gs://syrgoth/my- | 10 | BassetBranched | CNNTransferLearning |
| | | | model.epoch_5- | | | |
| | | | step_19885.pkl | | | |
| 325 | 0.5123253031152822 | L1KLmixed | gs://syrgoth/my- | 30 | BassetBranched | CNNTransferLearning |
| | | | model.epoch_5- | | | |
| | | | step_19885.pkl | | | |
| 326 | | L1KLmixed | gs://syrgoth/my- | 26 | BassetVL | CNNTransferLearning |
| | | | model.epoch_5- | | | |
| | | | step_19885.pkl | | | |
| 327 | 0.2100355455965437 | L1KLmixed | gs://syrgoth/my- | 41 | BassetBranched | CNNTransferLearning |
| | | | model.epoch_5- | | | |
| | | | step_19885.pkl | | | |
| 328 | | L1KLmixed | | | BassetVL | CNNBasicTraining |
| 329 | 0.4291413437949328 | L1KLmixed | gs://syrgoth/my- | 30 | BassetBranched | CNNTransferLearning |
| | | | model.epoch_5- | | | |
| | | | step_19885.pkl | | | |
| 330 | | L1KLmixed | gs://syrgoth/my- | 49 | BassetVL | CNNTransferLearning |
| | | | model.epoch_5- | | | |
| | | | step_19885.pkl | | | |
| 331 | | L1KLmixed | gs://syrgoth/my- | 57 | BassetVL | CNNTransferLearning |
| | | | model.epoch_5- | | | |
| | | | step_19885.pkl | | | |
| 332 | 0.40945422 | L1KLmixed | gs://syrgoth/my- | 27 | BassetBranched | CNNTransferLearning |
| | | | model.epoch_5- | | | |
| | | | step_19885.pkl | | | |
| 333 | 0.3654071989506303 | L1KLmixed | gs://syrgoth/my- | 42 | BassetBranched | CNNTransferLearning |
| | | | model.epoch_5- | | | |
| | | | step_19885.pkl | | | |
| 334 | 0.2461932864945035 | L1KLmixed | gs://syrgoth/my- | 41 | BassetBranched | CNNTransferLearning |
| | | | model.epoch_5- | | | |
| | | | step_19885.pkl | | | |
| 335 | 0.1386816697741569 | L1KLmixed | | | BassetBranched | CNNBasicTraining |
| 336 | 0.22100854 | MSEKLmixed | | | BassetBranched | CNNBasicTraining |
| 337 | 0.3695765086580481 | L1KLmixed | gs://syrgoth/my- | 50 | BassetBranched | CNNTransferLearning |
| | | | model.epoch_5- | | | |
| | | | step_19885.pkl | | | |
| 338 | 0.3180360253000116 | L1KLmixed | gs://syrgoth/my- | 22 | BassetBranched | CNNTransferLearning |
| | | | model.epoch_5- | | | |
| | | | step_19885.pkl | | | |
| 339 | | MSEKLmixed | | | BassetVL | CNNBasicTraining |
| 340 | 0.40472751 | L1KLmixed | gs://syrgoth/my- | 49 | BassetBranched | CNNTransferLearning |
| | | | model.epoch_5- | | | |
| | | | step_19885.pkl | | | |
| 341 | 0.3633127340347955 | L1KLmixed | gs://syrgoth/my- | 49 | BassetBranched | CNNTransferLearning |
| | | | model.epoch_5- | | | |
| | | | step_19885.pkl | | | |
| 342 | | MSEKLmixed | | | BassetVL | CNNBasicTraining |
| 343 | 0.5019360193101412 | L1KLmixed | gs://syrgoth/my- | 31 | BassetBranched | CNNTransferLearning |
| | | | model.epoch_5- | | | |
| | | | step_19885.pkl | | | |
| 344 | | L1KLmixed | | | BassetVL | CNNBasicTraining |
| 345 | 0.3760973410930088 | L1KLmixed | gs://syrgoth/my- | 30 | BassetBranched | CNNTransferLearning |
| | | | model.epoch_5- | | | |
| | | | step_19885.pkl | | | |
| 346 | | L1KLmixed | gs://syrgoth/my- | 46 | BassetVL | CNNTransferLearning |
| | | | model.epoch_5- | | | |
| | | | step_19885.pkl | | | |
| 347 | | L1KLmixed | gs://syrgoth/my- | 45 | BassetVL | CNNTransferLearning |
| | | | model.epoch_5- | | | |
| | | | step_19885.pkl | | | |
| 348 | 0.1295928542151724 | L1KLmixed | | | BassetBranched | CNNBasicTraining |
| 349 | | L1KLmixed | | | BassetVL | CNNBasicTraining |
| 350 | 0.4163206966658165 | L1KLmixed | gs://syrgoth/my- | 31 | BassetBranched | CNNTransferLearning |
| | | | model.epoch_5- | | | |
| | | | step_19885.pkl | | | |
| 351 | 0.4391960023796268 | L1KLmixed | gs://syrgoth/my- | 37 | BassetBranched | CNNTransferLearning |
| | | | model.epoch_5- | | | |
| | | | step_19885.pkl | | | |
| 352 | | L1KLmixed | gs://syrgoth/my- | 37 | BassetVL | CNNTransferLearning |
| | | | model.epoch_5- | | | |
| | | | step_19885.pkl | | | |
| 353 | 0.3039628033569987 | MSEKLmixed | | | BassetBranched | CNNBasicTraining |
| 354 | 0.5424143515616658 | L1KLmixed | gs://syrgoth/my- | 21 | BassetBranched | CNNTransferLearning |
| | | | model.epoch_5- | | | |
| | | | step_19885.pkl | | | |
| 355 | 0.05 | L1KLmixed | | | BassetBranched | CNNBasicTraining |
| 356 | 0.07981 | L1KLmixed | | | BassetBranched | CNNBasicTraining |
| 357 | 0.2395410139925942 | MSEKLmixed | gs://syrgoth/my- | 43 | BassetBranched | CNNTransferLearning |
| | | | model.epoch_5- | | | |
| | | | step_19885.pkl | | | |
| 358 | | L1KLmixed | gs://syrgoth/my- | 50 | BassetVL | CNNTransferLearning |
| | | | model.epoch_5- | | | |
| | | | step_19885.pkl | | | |
| 359 | 0.3331773047036576 | L1KLmixed | | | BassetBranched | CNNBasicTraining |
| 360 | 0.4225789814035663 | L1KLmixed | gs://syrgoth/my- | 33 | BassetBranched | CNNTransferLearning |
| | | | model.epoch_5- | | | |
| | | | step_19885.pkl | | | |
| 361 | 0.1275431706898486 | L1KLmixed | | | BassetBranched | CNNBasicTraining |
| 362 | 0.4041190059491187 | L1KLmixed | gs://syrgoth/my- | 31 | BassetBranched | CNNTransferLearning |
| | | | model.epoch_5- | | | |
| | | | step_19885.pkl | | | |
| 363 | 0.5257827706171863 | L1KLmixed | gs://syrgoth/my- | 20 | BassetBranched | CNNTransferLearning |
| | | | model.epoch_5- | | | |
| | | | step_19885.pkl | | | |
| 364 | | L1KLmixed | gs://syrgoth/my- | 45 | BassetVL | CNNTransferLearning |
| | | | model.epoch_5- | | | |
| | | | step_19885.pkl | | | |
| 365 | | L1KLmixed | gs://syrgoth/my- | 44 | BassetVL | CNNTransferLearning |
| | | | model.epoch_5- | | | |
| | | | step_19885.pkl | | | |
| 366 | 0.3119242792582852 | L1KLmixed | gs://syrgoth/my- | 28 | BassetBranched | CNNTransferLearning |
| | | | model.epoch_5- | | | |
| | | | step_19885.pkl | | | |
| 367 | | L1KLmixed | gs://syrgoth/my- | 49 | BassetVL | CNNTransferLearning |
| | | | model.epoch_5- | | | |
| | | | step_19885.pkl | | | |
| 368 | 0.1082567802798217 | L1KLmixed | | | BassetBranched | CNNBasicTraining |
| 369 | | L1KLmixed | | | BassetVL | CNNBasicTraining |
| 370 | 0.4362595387791459 | L1KLmixed | gs://syrgoth/my- | 29 | BassetBranched | CNNTransferLearning |
| | | | model.epoch_5- | | | |
| | | | step_19885.pkl | | | |
| 371 | 0.4393015430899498 | MSEKLmixed | | | BassetBranched | CNNBasicTraining |
| 372 | | L1KLmixed | gs://syrgoth/my- | 41 | BassetVL | CNNTransferLearning |
| | | | model.epoch_5- | | | |
| | | | step_19885.pkl | | | |
| 373 | | L1KLmixed | | | BassetVL | CNNBasicTraining |
| 374 | 0.3703572478706459 | MSEKLmixed | gs://syrgoth/my- | 41 | BassetBranched | CNNTransferLearning |
| | | | model.epoch_5- | | | |
| | | | step_19885.pkl | | | |
| 375 | | L1KLmixed | | | BassetVL | CNNBasicTraining |
| 376 | | MSEKLmixed | | | BassetVL | CNNBasicTraining |
| 377 | 0.1404715 | L1KLmixed | | | BassetBranched | CNNBasicTraining |
| 378 | | L1KLmixed | gs://syrgoth/my- | 44 | BassetVL | CNNTransferLearning |
| | | | model.epoch_5- | | | |
| | | | step_19885.pkl | | | |
| 379 | 0.05 | L1KLmixed | | | BassetBranched | CNNBasicTraining |
| 380 | | MSEKLmixed | | | BassetVL | CNNBasicTraining |
| 381 | 0.4752900532366484 | L1KLmixed | gs://syrgoth/my- | 22 | BassetBranched | CNNTransferLearning |
| | | | model.epoch_5- | | | |
| | | | step_19885.pkl | | | |
| 382 | 0.4010489866978929 | L1KLmixed | gs://syrgoth/my- | 28 | BassetBranched | CNNTransferLearning |
| | | | model.epoch_5- | | | |
| | | | step_19885.pkl | | | |
| 383 | 0.3456452571786925 | L1KLmixed | gs://syrgoth/my- | 41 | BassetBranched | CNNTransferLearning |
| | | | model.epoch_5- | | | |
| | | | step_19885.pkl | | | |
| 384 | 0.4963233419758096 | L1KLmixed | gs://syrgoth/my- | 23 | BassetBranched | CNNTransferLearning |
| | | | model.epoch_5- | | | |
| | | | step_19885.pkl | | | |
| 385 | | L1KLmixed | gs://syrgoth/my- | 60 | BassetVL | CNNTransferLearning |
| | | | model.epoch_5- | | | |
| | | | step_19885.pkl | | | |
| 386 | 0.5219938050420329 | MSEKLmixed | gs://syrgoth/my- | 56 | BassetBranched | CNNTransferLearning |
| | | | model.epoch_5- | | | |
| | | | step_19885.pkl | | | |
| 387 | 0.43065007 | L1KLmixed | gs://syrgoth/my- | 28 | BassetBranched | CNNTransferLearning |
| | | | model.epoch_5- | | | |
| | | | step_19885.pkl | | | |
| 388 | 0.07308526 | L1KLmixed | | | BassetBranched | CNNBasicTraining |
| 389 | 0.4584471408502827 | MSEKLmixed | | | BassetBranched | CNNBasicTraining |
| 390 | | MSEKLmixed | | | BassetVL | CNNBasicTraining |
| 391 | | MSEKLmixed | gs://syrgoth/my- | 15 | BassetVL | CNNTransferLearning |
| | | | model.epoch_5- | | | |
| | | | step_19885.pkl | | | |
| 392 | | L1KLmixed | gs://syrgoth/my- | 58 | BassetVL | CNNTransferLearning |
| | | | model.epoch_5- | | | |
| | | | step_19885.pkl | | | |
| 393 | 0.4881147615220848 | L1KLmixed | gs://syrgoth/my- | 28 | BassetBranched | CNNTransferLearning |
| | | | model.epoch_5- | | | |
| | | | step_19885.pkl | | | |
| 394 | 0.3537636950888108 | L1KLmixed | gs://syrgoth/my- | 13 | BassetBranched | CNNTransferLearning |
| | | | model.epoch_5- | | | |
| | | | step_19885.pkl | | | |
| 395 | 0.3806274271267775 | L1KLmixed | gs://syrgoth/my- | 54 | BassetBranched | CNNTransferLearning |
| | | | model.epoch_5- | | | |
| | | | step_19885.pkl | | | |
| 396 | | L1KLmixed | | | BassetVL | CNNBasicTraining |
| 397 | | L1KLmixed | gs://syrgoth/my- | 59 | BassetVL | CNNTransferLearning |
| | | | model.epoch_5- | | | |
| | | | step_19885.pkl | | | |
| 398 | | MSEKLmixed | | | BassetVL | CNNBasicTraining |
| 399 | | L1KLmixed | gs://syrgoth/my- | 20 | BassetVL | CNNTransferLearning |
| | | | model.epoch_5- | | | |
| | | | step_19885.pkl | | | |
| 400 | 0.4290866310022818 | L1KLmixed | gs://syrgoth/my- | 22 | BassetBranched | CNNTransferLearning |
| | | | model.epoch_5- | | | |
| | | | step_19885.pkl | | | |
| 401 | 0.4140304107438479 | L1KLmixed | gs://syrgoth/my- | 31 | BassetBranched | CNNTransferLearning |
| | | | model.epoch_5- | | | |
| | | | step_19885.pkl | | | |
| 402 | 0.3879775288767202 | MSEKLmixed | gs://syrgoth/my- | 56 | BassetBranched | CNNTransferLearning |
| | | | model.epoch_5- | | | |
| | | | step_19885.pkl | | | |
| 403 | 0.4303621897263714 | L1KLmixed | gs://syrgoth/my- | 35 | BassetBranched | CNNTransferLearning |
| | | | model.epoch_5- | | | |
| | | | step_19885.pkl | | | |
| 404 | 0.3543409726847017 | L1KLmixed | gs://syrgoth/my- | 43 | BassetBranched | CNNTransferLearning |
| | | | model.epoch_5- | | | |
| | | | step_19885.pkl | | | |
| 405 | 0.5447229759996803 | L1KLmixed | gs://syrgoth/my- | 40 | BassetBranched | CNNTransferLearning |
| | | | model.epoch_5- | | | |
| | | | step_19885.pkl | | | |
| 406 | 0.5696829050617286 | L1KLmixed | gs://syrgoth/my- | 27 | BassetBranched | CNNTransferLearning |
| | | | model.epoch_5- | | | |
| | | | step_19885.pkl | | | |
| 407 | 0.05116386 | L1KLmixed | | | BassetBranched | CNNBasicTraining |
| 408 | 0.4523574609156489 | L1KLmixed | gs://syrgoth/my- | 25 | BassetBranched | CNNTransferLearning |
| | | | model.epoch_5- | | | |
| | | | step_19885.pkl | | | |
| 409 | 0.39827845 | L1KLmixed | gs://syrgoth/my- | 21 | BassetBranched | CNNTransferLearning |
| | | | model.epoch_5- | | | |
| | | | step_19885.pkl | | | |
| 410 | 0.4899004908291405 | L1KLmixed | gs://syrgoth/my- | 33 | BassetBranched | CNNTransferLearning |
| | | | model.epoch_5- | | | |
| | | | step_19885.pkl | | | |
| 411 | 0.1616551342706564 | L1KLmixed | | | BassetBranched | CNNBasicTraining |
| 412 | | L1KLmixed | | | BassetVL | CNNBasicTraining |
| 413 | 0.05285301 | L1KLmixed | | | BassetBranched | CNNBasicTraining |
| 414 | | L1KLmixed | gs://syrgoth/my- | 40 | BassetVL | CNNTransferLearning |
| | | | model.epoch_5- | | | |
| | | | step_19885.pkl | | | |
| 415 | 0.05696916 | L1KLmixed | gs://syrgoth/my- | 24 | BassetBranched | CNNTransferLearning |
| | | | model.epoch_5- | | | |
| | | | step_19885.pkl | | | |
| 416 | 0.4345945407475841 | L1KLmixed | gs://syrgoth/my- | 24 | BassetBranched | CNNTransferLearning |
| | | | model.epoch_5- | | | |
| | | | step_19885.pkl | | | |
| 417 | | L1KLmixed | gs://syrgoth/my- | 39 | BassetVL | CNNTransferLearning |
| | | | model.epoch_5- | | | |
| | | | step_19885.pkl | | | |
| 418 | 0.5449122698347293 | L1KLmixed | gs://syrgoth/my- | 39 | BassetBranched | CNNTransferLearning |
| | | | model.epoch_5- | | | |
| | | | step_19885.pkl | | | |
| 419 | 0.4279638410767037 | L1KLmixed | gs://syrgoth/my- | 31 | BassetBranched | CNNTransferLearning |
| | | | model.epoch_5- | | | |
| | | | step_19885.pkl | | | |
| 420 | | L1KLmixed | gs://syrgoth/my- | 59 | BassetVL | CNNTransferLearning |
| | | | model.epoch_5- | | | |
| | | | step_19885.pkl | | | |
| 421 | | L1KLmixed | gs://syrgoth/my- | 53 | BassetVL | CNNTransferLearning |
| | | | model.epoch_5- | | | |
| | | | step_19885.pkl | | | |
| 422 | 0.4472501201418772 | L1KLmixed | gs://syrgoth/my- | 28 | BassetBranched | CNNTransferLearning |
| | | | model.epoch_5- | | | |
| | | | step_19885.pkl | | | |
| 423 | 0.4671609507231666 | L1KLmixed | gs://syrgoth/my- | 28 | BassetBranched | CNNTransferLearning |
| | | | model.epoch_5- | | | |
| | | | step_19885.pkl | | | |
| 424 | | L1KLmixed | gs://syrgoth/my- | 40 | BassetVL | CNNTransferLearning |
| | | | model.epoch_5- | | | |
| | | | step_19885.pkl | | | |
| 425 | 0.2708553089493036 | MSEKLmixed | gs://syrgoth/my- | 36 | BassetBranched | CNNTransferLearning |
| | | | model.epoch_5- | | | |
| | | | step_19885.pkl | | | |
| 426 | | L1KLmixed | gs://syrgoth/my- | 44 | BassetVL | CNNTransferLearning |
| | | | model.epoch_5- | | | |
| | | | step_19885.pkl | | | |
| 427 | | L1KLmixed | gs://syrgoth/my- | 46 | BassetVL | CNNTransferLearning |
| | | | model.epoch_5- | | | |
| | | | step_19885.pkl | | | |
| 428 | 0.2589136848515133 | L1KLmixed | | | BassetBranched | CNNBasicTraining |
| 429 | 0.4814119694841025 | L1KLmixed | gs://syrgoth/my- | 33 | BassetBranched | CNNTransferLearning |
| | | | model.epoch_5- | | | |
| | | | step_19885.pkl | | | |
| 430 | 0.3597544064981436 | L1KLmixed | gs://syrgoth/my- | 40 | BassetBranched | CNNTransferLearning |
| | | | model.epoch_5- | | | |
| | | | step_19885.pkl | | | |
| 431 | 0.5143944527496688 | L1KLmixed | gs://syrgoth/my- | 29 | BassetBranched | CNNTransferLearning |
| | | | model.epoch_5- | | | |
| | | | step_19885.pkl | | | |
| 432 | | L1KLmixed | | | BassetVL | CNNBasicTraining |
| 433 | | L1KLmixed | gs://syrgoth/my- | 59 | BassetVL | CNNTransferLearning |
| | | | model.epoch_5- | | | |
| | | | step_19885.pkl | | | |
| 434 | 0.1315620604264926 | MSEKLmixed | | | BassetBranched | CNNBasicTraining |
| 435 | 0.05315233 | L1KLmixed | | | BassetBranched | CNNBasicTraining |
| 436 | 0.4575063301037451 | L1KLmixed | gs://syrgoth/my- | 30 | BassetBranched | CNNTransferLearning |
| | | | model.epoch_5- | | | |
| | | | step_19885.pkl | | | |
| 437 | | L1KLmixed | gs://syrgoth/my- | 46 | BassetVL | CNNTransferLearning |
| | | | model.epoch_5- | | | |
| | | | step_19885.pkl | | | |
| 438 | | L1KLmixed | gs://syrgoth/my- | 40 | BassetVL | CNNTransferLearning |
| | | | model.epoch_5- | | | |
| | | | step_19885.pkl | | | |
| 439 | 0.1262838186860034 | L1KLmixed | | | BassetBranched | CNNBasicTraining |
| 440 | | L1KLmixed | gs://syrgoth/my- | 49 | BassetVL | CNNTransferLearning |
| | | | model.epoch_5- | | | |
| | | | step_19885.pkl | | | |
| 441 | 0.4160225879946357 | L1KLmixed | gs://syrgoth/my- | 31 | BassetBranched | CNNTransferLearning |
| | | | model.epoch_5- | | | |
| | | | step_19885.pkl | | | |
| 442 | 0.6356996149344187 | L1KLmixed | gs://syrgoth/my- | 19 | BassetBranched | CNNTransferLearning |
| | | | model.epoch_5- | | | |
| | | | step_19885.pkl | | | |
| 443 | | L1KLmixed | gs://syrgoth/my- | 60 | BassetVL | CNNTransferLearning |
| | | | model.epoch_5- | | | |
| | | | step_19885.pkl | | | |
| 444 | | L1KLmixed | | | BassetVL | CNNBasicTraining |
| 445 | | L1KLmixed | gs://syrgoth/my- | 51 | BassetVL | CNNTransferLearning |
| | | | model.epoch_5- | | | |
| | | | step_19885.pkl | | | |
| 446 | | L1KLmixed | gs://syrgoth/my- | 48 | BassetVL | CNNTransferLearning |
| | | | model.epoch_5- | | | |
| | | | step_19885.pkl | | | |
| 447 | 0.4864151965259362 | L1KLmixed | gs://syrgoth/my- | 34 | BassetBranched | CNNTransferLearning |
| | | | model.epoch_5- | | | |
| | | | step_19885.pkl | | | |
| 448 | 0.4492932480214883 | L1KLmixed | gs://syrgoth/my- | 28 | BassetBranched | CNNTransferLearning |
| | | | model.epoch_5- | | | |
| | | | step_19885.pkl | | | |
| 449 | | L1KLmixed | gs://syrgoth/my- | 46 | BassetVL | CNNTransferLearning |
| | | | model.epoch_5- | | | |
| | | | step_19885.pkl | | | |
| 450 | 0.4568292372759414 | L1KLmixed | gs://syrgoth/my- | 27 | BassetBranched | CNNTransferLearning |
| | | | model.epoch_5- | | | |
| | | | step_19885.pkl | | | |
| 451 | | L1KLmixed | | | BassetVL | CNNBasicTraining |
| 452 | | L1KLmixed | gs://syrgoth/my- | 51 | BassetVL | CNNTransferLearning |
| | | | model.epoch_5- | | | |
| | | | step_19885.pkl | | | |
| 453 | | L1KLmixed | gs://syrgoth/my- | 7 | BassetVL | CNNTransferLearning |
| | | | model.epoch_5- | | | |
| | | | step_19885.pkl | | | |
| 454 | | L1KLmixed | gs://syrgoth/my- | 38 | BassetVL | CNNTransferLearning |
| | | | model.epoch_5- | | | |
| | | | step_19885.pkl | | | |
| 455 | 0.4616960178513773 | L1KLmixed | gs://syrgoth/my- | 13 | BassetBranched | CNNTransferLearning |
| | | | model.epoch_5- | | | |
| | | | step_19885.pkl | | | |
| 456 | | L1KLmixed | gs://syrgoth/my- | 54 | BassetVL | CNNTransferLearning |
| | | | model.epoch_5- | | | |
| | | | step_19885.pkl | | | |
| 457 | | L1KLmixed | gs://syrgoth/my- | 25 | BassetVL | CNNTransferLearning |
| | | | model.epoch_5- | | | |
| | | | step_19885.pkl | | | |
| 458 | | L1KLmixed | gs://syrgoth/my- | 36 | BassetVL | CNNTransferLearning |
| | | | model.epoch_5- | | | |
| | | | step_19885.pkl | | | |
| 459 | 0.48102134 | L1KLmixed | gs://syrgoth/my- | 36 | BassetBranched | CNNTransferLearning |
| | | | model.epoch_5- | | | |
| | | | step_19885.pkl | | | |
| 460 | 0.05 | L1KLmixed | | | BassetBranched | CNNBasicTraining |
| 461 | | L1KLmixed | gs://syrgoth/my- | 41 | BassetVL | CNNTransferLearning |
| | | | model.epoch_5- | | | |
| | | | step_19885.pkl | | | |
| 462 | | L1KLmixed | gs://syrgoth/my- | 47 | BassetVL | CNNTransferLearning |
| | | | model.epoch_5- | | | |
| | | | step_19885.pkl | | | |
| 463 | 0.5813728847121891 | L1KLmixed | gs://syrgoth/my- | 42 | BassetBranched | CNNTransferLearning |
| | | | model.epoch_5- | | | |
| | | | step_19885.pkl | | | |
| 464 | 0.1315077785901701 | L1KLmixed | gs://syrgoth/my- | 60 | BassetBranched | CNNTransferLearning |
| | | | model.epoch_5- | | | |
| | | | step_19885.pkl | | | |
| 465 | | L1KLmixed | gs://syrgoth/my- | 39 | BassetVL | CNNTransferLearning |
| | | | model.epoch_5- | | | |
| | | | step_19885.pkl | | | |
| 466 | | L1KLmixed | gs://syrgoth/my- | 22 | BassetVL | CNNTransferLearning |
| | | | model.epoch_5- | | | |
| | | | step_19885.pkl | | | |
| 467 | 0.4621287615769158 | L1KLmixed | gs://syrgoth/my- | 26 | BassetBranched | CNNTransferLearning |
| | | | model.epoch_5- | | | |
| | | | step_19885.pkl | | | |
| 468 | | L1KLmixed | gs://syrgoth/my- | 58 | BassetVL | CNNTransferLearning |
| | | | model.epoch_5- | | | |
| | | | step_19885.pkl | | | |
| 469 | 0.4096056271222179 | MSEKLmixed | gs://syrgoth/my- | 40 | BassetBranched | CNNTransferLearning |
| | | | model.epoch_5- | | | |
| | | | step_19885.pkl | | | |
| 470 | 0.3664419461382699 | MSEKLmixed | gs://syrgoth/my- | 48 | BassetBranched | CNNTransferLearning |
| | | | model.epoch_5- | | | |
| | | | step_19885.pkl | | | |
| 471 | 0.48621636 | L1KLmixed | gs://syrgoth/my- | 24 | BassetBranched | CNNTransferLearning |
| | | | model.epoch_5- | | | |
| | | | step_19885.pkl | | | |
| 472 | | L1KLmixed | gs://syrgoth/my- | 31 | BassetVL | CNNTransferLearning |
| | | | model.epoch_5- | | | |
| | | | step_19885.pkl | | | |
| 473 | 0.4250076956800191 | L1KLmixed | gs://syrgoth/my- | 38 | BassetBranched | CNNTransferLearning |
| | | | model.epoch_5- | | | |
| | | | step_19885.pkl | | | |
| 474 | | L1KLmixed | gs://syrgoth/my- | 46 | BassetVL | CNNTransferLearning |
| | | | model.epoch_5- | | | |
| | | | step_19885.pkl | | | |
| 475 | 0.6453874107634983 | L1KLmixed | gs://syrgoth/my- | 16 | BassetBranched | CNNTransferLearning |
| | | | model.epoch_5- | | | |
| | | | step_19885.pkl | | | |
| 476 | 0.2309627992390157 | MSEKLmixed | gs://syrgoth/my- | 34 | BassetBranched | CNNTransferLearning |
| | | | model.epoch_5- | | | |
| | | | step_19885.pkl | | | |
| 477 | | L1KLmixed | gs://syrgoth/my- | 57 | BassetVL | CNNTransferLearning |
| | | | model.epoch_5- | | | |
| | | | step_19885.pkl | | | |
| 478 | | L1KLmixed | gs://syrgoth/my- | 34 | BassetVL | CNNTransferLearning |
| | | | model.epoch_5- | | | |
| | | | step_19885.pkl | | | |
| 479 | | MSEKLmixed | gs://syrgoth/my- | 24 | BassetVL | CNNTransferLearning |
| | | | model.epoch_5- | | | |
| | | | step_19885.pkl | | | |
| 480 | | L1KLmixed | gs://syrgoth/my- | 59 | BassetVL | CNNTransferLearning |
| | | | model.epoch_5- | | | |
| | | | step_19885.pkl | | | |
| 481 | 0.2302184911226216 | L1KLmixed | | | BassetBranched | CNNTransferLearning |
| 482 | | L1KLmixed | | | BassetVL | CNNBasicTraining |
| 483 | 0.4548607559719325 | L1KLmixed | gs://syrgoth/my- | 18 | BassetBranched | CNNTransferLearning |
| | | | model.epoch_5- | | | |
| | | | step_19885.pkl | | | |
| 484 | | L1KLmixed | | | BassetVL | CNNBasicTraining |
| 485 | | L1KLmixed | gs://syrgoth/my- | 39 | BassetVL | CNNTransferLearning |
| | | | model.epoch_5- | | | |
| | | | step_19885.pkl | | | |
| 486 | 0.5510061299912571 | L1KLmixed | gs://syrgoth/my- | 40 | BassetBranched | CNNTransferLearning |
| | | | model.epoch_5- | | | |
| | | | step_19885.pkl | | | |
| 487 | 0.05022254 | L1KLmixed | | | BassetBranched | CNNBasicTraining |
| 488 | | L1KLmixed | | | BassetVL | CNNBasicTraining |
| 489 | | L1KLmixed | gs://syrgoth/my- | 41 | BassetVL | CNNTransferLearning |
| | | | model.epoch_5- | | | |
| | | | step_19885.pkl | | | |
| 490 | 0.3800969667215317 | L1KLmixed | gs://syrgoth/my- | 26 | BassetBranched | CNNTransferLearning |
| | | | model.epoch_5- | | | |
| | | | step_19885.pkl | | | |
| 491 | | L1KLmixed | gs://syrgoth/my- | 39 | BassetVL | CNNTransferLearning |
| | | | model.epoch_5- | | | |
| | | | step_19885.pkl | | | |
| 492 | | L1KLmixed | gs://syrgoth/my- | 50 | BassetVL | CNNTransferLearning |
| | | | model.epoch_5- | | | |
| | | | step_19885.pkl | | | |

| Row ID | lr | weight_decay | amsgrad | T_0 | beta | betas |
|---|---|---|---|---|---|---|
| 1 | 0.00107252 | 0.00014562 | FALSE | 8884 | 1.0013121665830635 | [0.9037276610467211, |
| | | | | | | 0.9041494581425669] |
| 2 | 0.00212223 | 0.00023738 | TRUE | 2989 | 0.2 | [0.8412842739400004, |
| | | | | | | 0.9600483641249183] |
| 3 | 0.01 | 1.6442845805384335e−05 | TRUE | 2055 | 1.079909333789426 | [0.8000000000000002, |
| | | | | | | 0.8580983969691933] |
| 4 | 0.00083912 | 0.00022512 | FALSE | 14914 | 0.9370852578618748 | [0.9264948443531243, |
| | | | | | | 0.8251684953948799] |
| 5 | 0.00206645 | 0.00021465 | TRUE | 3222 | 0.2055522875457384 | [0.8470722952879045, |
| | | | | | | 0.9165489695569012] |
| 6 | 0.00141465 | 0.0002815 | TRUE | 7512 | 0.3168564478868103 | [0.8902953202616644, |
| | | | | | | 0.9999] |
| 7 | 0.00943885 | 0.00013048 | FALSE | 11209 | 0.31603797 | [0.8027821929876005, |
| | | | | | | 0.9446350535718394] |
| 8 | 0.00221723 | 0.00025713 | TRUE | 4763 | 2.737500735722491 | [0.9514919176365978, |
| | | | | | | 0.8460988323800336] |
| 9 | 0.00144318 | 0.00027415 | TRUE | 2916 | 0.453737748 | [0.9312750971835905, |
| | | | | | | 0.8005413882619896] |
| 10 | 0.00203856 | 0.00023424 | TRUE | 3128 | 0.2 | [0.853629704975785, |
| | | | | | | 0.9392499392464815] |
| 11 | 0.00204849 | 0.00019508 | TRUE | 2851 | 1.990005367578669 | [0.9541889058419742, |
| | | | | | | 0.8037678490641568] |
| 12 | 0.00192816 | 0.00026028 | TRUE | 4271 | 2.533953564401113 | [0.949873083420064, |
| | | | | | | 0.8000000000000002] |
| 13 | 0.00203717 | 0.00021156 | TRUE | 5020 | 0.962065684 | [0.934574740036491, |
| | | | | | | 0.8769788467602946] |
| 14 | 0.00090212 | 0.00028854 | TRUE | 24007 | 1.9263546857852103 | [0.9315284375302461, |
| | | | | | | 0.8002182335017651] |
| 15 | 0.00177205 | 0.00025151 | TRUE | 6672 | 0.7434145293562109 | [0.9370129377328579, |
| | | | | | | 0.8817598075054247] |
| 16 | 0.00210375 | 0.00017053 | TRUE | 2110 | 3.784169343635343 | [0.9548195525232648, |
| | | | | | | 0.8001189489290699] |
| 17 | 0.00198119 | 0.00023829 | TRUE | 3901 | 0.2093632862185297 | [0.8468797211734592, |
| | | | | | | 0.9548741178843074] |
| 18 | 0.00058555 | 6.813058298039242e−05 | TRUE | 13709 | 1.063664228981821 | [0.9056515382749053, |
| | | | | | | 0.8757024115532658] |
| 19 | 0.00221966 | 0.00025896 | TRUE | 2048 | 3.6450212110776152 | [0.9033096565934918, |
| | | | | | | 0.816149433554705] |
| 20 | 0.00206553 | 0.00023008 | TRUE | 3045 | 0.2 | [0.8478014202330515, |
| | | | | | | 0.9375885918405165] |
| 21 | 0.00191042 | 0.0001834 | TRUE | 6346 | 1.6793235342753825 | [0.942773199056052, |
| | | | | | | 0.8490723829063853] |
| 22 | 0.00212111 | 0.00020171 | TRUE | 2205 | 1.521220339512037 | [0.964099815592594, |
| | | | | | | 0.8067691162419544] |
| 23 | 0.00064927 | 0.00046304 | TRUE | 10634 | 0.2413659335758672 | [0.9194351368142883, |
| | | | | | | 0.803625120197886] |
| 24 | 0.00136103 | 0.00012066 | TRUE | 2048 | 1.0046325578561988 | [0.9667540273438266, |
| | | | | | | 0.9998709231848714] |
| 25 | 0.00208478 | 0.00023123 | TRUE | 3770 | 0.2 | [0.8482056476741471, |
| | | | | | | 0.9999] |
| 26 | 0.00108913 | 0.00022905 | TRUE | 5297 | 1.0320039612792005 | [0.8935357576128001, |
| | | | | | | 0.9010914896888753] |
| 27 | 0.00072466 | 0.00016696 | TRUE | 12943 | 0.9741854560285448 | [0.9437579435708604, |
| | | | | | | 0.887004271236215] |
| 28 | 0.00141312 | 0.00018364 | TRUE | 18913 | 0.3190118457223561 | [0.8663193386581503, |
| | | | | | | 0.9769649297044349] |
| 29 | 0.00175082 | 0.00020073 | TRUE | 32110 | 1.0204291138903034 | [0.9455266667398735, |
| | | | | | | 0.8996956273700046] |
| 30 | 0.00090498 | 3.5715304588269204e−05 | FALSE | 4749 | 1.9564704996886204 | [0.997273234928916, |
| | | | | | | 0.965364983380587] |
| 31 | 0.00096593 | 0.00020802 | TRUE | 2132 | 0.3187234229513374 | [0.8585726076082126, |
| | | | | | | 0.9628943280682023] |
| 32 | 0.00199229 | 0.00023108 | TRUE | 3433 | 0.2 | [0.8000000000000002, |
| | | | | | | 0.9999] |
| 33 | 0.00241339 | 6.844076756115446e−05 | FALSE | 17574 | 1.0510300174446507 | [0.9552896209295152, |
| | | | | | | 0.8460749522523828] |
| 34 | 0.00233344 | 0.00035933 | TRUE | 7004 | 0.9755247672632734 | [0.9533582715603637, |
| | | | | | | 0.8359243772711261] |
| 35 | 0.00183006 | 0.00021569 | TRUE | 3552 | 0.7865990230672733 | [0.9506802460664354, |
| | | | | | | 0.8320499345494324] |
| 36 | 0.0001159 | 2.5124643661548227e−05 | FALSE | 2048 | 0.8671623787998819 | [0.9016798930561275, |
| | | | | | | 0.8028661674566973] |
| 37 | 0.00236371 | 0.00028933 | TRUE | 2627 | 4.325990870247543 | [0.958749090920905, |
| | | | | | | 0.8636115516762066] |
| 38 | 0.00987746 | 0.00019375 | FALSE | 2052 | 1.0999860723595636 | [0.8000000000000002, |
| | | | | | | 0.8208071508686474] |
| 39 | 0.00147564 | 0.00026276 | TRUE | 6235 | 0.9585524464224164 | [0.9978047070542482, |
| | | | | | | 0.9064829384217509] |
| 40 | 0.00101151 | 9.47146930725473e−05 | FALSE | 15335 | 0.5618452296683608 | [0.906768973039806, |
| | | | | | | 0.9002043550641564] |
| 41 | 0.01 | 1.3238434548478024e−05 | TRUE | 2090 | 0.5677361028217186 | [0.8000000000000002, |
| | | | | | | 0.8287056176233385] |
| 42 | 0.00254172 | 0.00012409 | TRUE | 3198 | 0.2002002027205982 | [0.8356177307179509, |
| | | | | | | 0.9587669062856029] |
| 43 | 0.00472602 | 0.00098928 | FALSE | 2586 | 4.638666596089936 | [0.8022519291452622, |
| | | | | | | 0.8798692706073398] |
| 44 | 0.00204581 | 0.00024007 | TRUE | 2832 | 0.2 | [0.8569764996514223, |
| | | | | | | 0.9232373053730729] |
| 45 | 0.00183315 | 6.317629916462515e−05 | FALSE | 10938 | 1.5353452723539764 | [0.9225614929787525, |
| | | | | | | 0.9269161345961707] |
| 46 | 0.0016218 | 3.55499842811678e−05 | FALSE | 7717 | 2.445252578786903 | [0.9033596943588349, |
| | | | | | | 0.9345480052927999] |
| 47 | 0.00141908 | 0.00025742 | TRUE | 5735 | 0.2813233912092243 | [0.956914195173362, |
| | | | | | | 0.9422820550282409] |
| 48 | 0.0008021 | 0.00014865 | TRUE | 7038 | 0.9192600135424503 | [0.9153851533838852, |
| | | | | | | 0.8254113249382486] |
| 49 | 0.00195169 | 0.00025234 | TRUE | 2048 | 0.3151811187768616 | [0.8437396050576431, |
| | | | | | | 0.9045746133764433] |
| 50 | 0.00154856 | 0.00023159 | FALSE | 6637 | 0.8744528719043387 | [0.9326398180116798, |
| | | | | | | 0.8782475518019319] |
| 51 | 0.00999091 | 1.003756931235674e−05 | TRUE | 2048 | 3.429225443784137 | [0.8168634463845814, |
| | | | | | | 0.8427692033052794] |
| 52 | 0.00187404 | 0.00026636 | TRUE | 4345 | 2.339160113775064 | [0.9538310901762066, |
| | | | | | | 0.8022304202485961] |
| 53 | 0.00193292 | 0.00024328 | TRUE | 4155 | 0.2015798522276965 | [0.8550844444851641, |
| | | | | | | 0.9145831629412235] |
| 54 | 0.00161072 | 0.00024554 | TRUE | 5795 | 0.3103027534134119 | [0.8232247725580437, |
| | | | | | | 0.9999] |
| 55 | 0.00010012 | 0.00053847 | FALSE | 28529 | 4.341406511394386 | [0.8062616845290816, |
| | | | | | | 0.9262726689822832] |
| 56 | 0.0016481 | 0.001 | TRUE | 10185 | 0.6901780460300206 | [0.8591639916556244, |
| | | | | | | 0.8755754687776208] |
| 57 | 0.0014838 | 0.00031065 | TRUE | 2048 | 0.267956911 | [0.8340984482541494, |
| | | | | | | 0.9945263137603022] |
| 58 | 0.00187352 | 0.00023822 | TRUE | 3242 | 2.007336502 | [0.9560940238139183, |
| | | | | | | 0.8024297217442923] |
| 59 | 0.00201585 | 0.00019077 | TRUE | 2048 | 2.2937948741938303 | [0.9499555009459703, |
| | | | | | | 0.8033122312422204] |
| 60 | 0.00208099 | 0.00019727 | TRUE | 2696 | 2.485618212625176 | [0.9464890293006014, |
| | | | | | | 0.8019665469162125] |
| 61 | 0.00174909 | 0.00012522 | TRUE | 2048 | 0.3443536130132954 | [0.9046236219612106, |
| | | | | | | 0.9791630983012988] |
| 62 | 0.00052727 | 0.00011702 | FALSE | 12676 | 0.5027022322221513 | [0.8977445633490422, |
| | | | | | | 0.8928932527339819] |
| 63 | 0.00198318 | 0.00020746 | TRUE | 2724 | 3.170568655415821 | [0.9540400136005054, |
| | | | | | | 0.8041315342504137] |
| 64 | 0.00215911 | 0.00030223 | TRUE | 2399 | 0.2 | [0.8886209906208205, |
| | | | | | | 0.9162331938478894] |
| 65 | 0.00181341 | 9.697365621627012e−05 | TRUE | 2454 | 0.4471206527575207 | [0.9568367482993249, |
| | | | | | | 0.9692994563564861] |
| 66 | 0.00169686 | 0.00019438 | TRUE | 4765 | 0.714102429 | [0.9405829430456496, |
| | | | | | | 0.9104180992337946] |
| 67 | 0.0020778 | 0.00025349 | TRUE | 4082 | 1.7912491320403303 | [0.9457624587089066, |
| | | | | | | 0.8597362077690449] |
| 68 | 0.00149574 | 0.00026793 | TRUE | 4225 | 0.3140104838679494 | [0.889796222075978, |
| | | | | | | 0.9999] |
| 69 | 0.00205352 | 0.00023565 | TRUE | 5470 | 3.088322402292682 | [0.9475942844614795, |
| | | | | | | 0.8000000000000002] |
| 70 | 0.00207722 | 0.00023782 | TRUE | 4140 | 0.2110873306074968 | [0.8422007951133597, |
| | | | | | | 0.933421709843558] |
| 71 | 0.00186907 | 0.00026429 | TRUE | 3824 | 2.645403782607953 | [0.9482441654175875, |
| | | | | | | 0.8025835196148035] |
| 72 | 0.0098956 | 0.00010274 | TRUE | 3509 | 0.3686726675725216 | [0.8002909711065209, |
| | | | | | | 0.8205538154642033] |
| 73 | 0.00256676 | 8.299132947908603e−05 | TRUE | 9525 | 0.816667713 | [0.9195989837668099, |
| | | | | | | 0.8000000000000002] |
| 74 | 0.00997985 | 0.00014569 | FALSE | 24625 | 0.5455848025478808 | [0.8483079244037813, |
| | | | | | | 0.8000000000000002] |
| 75 | 0.00212623 | 0.00019594 | TRUE | 2143 | 0.2 | [0.8816372701806863, |
| | | | | | | 0.9031717228698966] |
| 76 | 0.00183074 | 0.00022172 | FALSE | 2645 | 2.435735979801242 | [0.9461620610179613, |
| | | | | | | 0.8492821608794461] |
| 77 | 0.00010707 | 0.00030398 | FALSE | 65536 | 2.955309654699069 | [0.8153186869373202, |
| | | | | | | 0.9455329928243384] |
| 78 | 0.00204453 | 0.00017966 | TRUE | 2252 | 3.781955263189385 | [0.945299522686097, |
| | | | | | | 0.8039774014426411] |
| 79 | 0.00671641 | 2.0693912249133628e−05 | TRUE | 12252 | 0.7861633109542052 | [0.8000000000000002, |
| | | | | | | 0.9478897911118735] |
| 80 | 0.00177621 | 0.0001822 | TRUE | 4977 | 1.1381139113637933 | [0.9441949949342753, |
| | | | | | | 0.8530994045416938] |
| 81 | 0.00189918 | 0.00019403 | TRUE | 2829 | 3.080984839557133 | [0.9434746236732581, |
| | | | | | | 0.8012197555761466] |
| 82 | 0.00208561 | 0.00017523 | TRUE | 2665 | 4.147335751453281 | [0.937159763337735, |
| | | | | | | 0.8000560469540585] |
| 83 | 0.00187266 | 0.00025108 | TRUE | 4865 | 2.033390147818729 | [0.9580877683806612, |
| | | | | | | 0.8006582594047105] |
| 84 | 0.00934569 | 0.00015002 | TRUE | 4821 | 0.5924233981188345 | [0.8017523622803153, |
| | | | | | | 0.8725314920980449] |
| 85 | 0.00192803 | 0.00021127 | TRUE | 3482 | 2.0056448610439985 | [0.9580585427626991, |
| | | | | | | 0.8047277576165102] |
| 86 | 0.00160011 | 0.00016207 | TRUE | 2532 | 0.2956613124918137 | [0.8217387566800157, |
| | | | | | | 0.9994815748005619] |
| 87 | 0.00200158 | 0.00021709 | TRUE | 3047 | 0.2 | [0.8933209567822509, |
| | | | | | | 0.8000000000000002] |
| 88 | 0.00206208 | 0.00023539 | TRUE | 3267 | 0.2 | [0.8540307478981208, |
| | | | | | | 0.9310931273617786] |
| 89 | 0.00294427 | 0.00025196 | TRUE | 3977 | 0.2 | [0.8466943600370971, |
| | | | | | | 0.9414189969560947] |
| 90 | 0.001 | 0.0001 | FALSE | 11585 | 1 | [0.9055175314777241, |
| | | | | | | 0.9055175314777241] |
| 91 | 0.00305251 | 9.541140636093356e−05 | FALSE | 11919 | 0.6870850397805762 | [0.8351603911740684, |
| | | | | | | 0.9358441018205317] |
| 92 | 0.00200769 | 0.00024157 | TRUE | 4240 | 0.2487231357754866 | [0.8565293064726871, |
| | | | | | | 0.9360809270278696] |
| 93 | 0.00221421 | 0.00019931 | TRUE | 2374 | 2.724893283147204 | [0.9180943324826658, |
| | | | | | | 0.8000000000000002] |
| 94 | 0.00183212 | 0.00018979 | TRUE | 4689 | 1.054218015102799 | [0.9429839920305733, |
| | | | | | | 0.8565956853024088] |
| 95 | 0.00182498 | 0.00027233 | TRUE | 2048 | 2.542284308455172 | [0.9555204111587772, |
| | | | | | | 0.8344327070460842] |
| 96 | 0.00283865 | 0.001 | TRUE | 4465 | 0.7550458537411148 | [0.853886088347632, |
| | | | | | | 0.9094067981726359] |
| 97 | 0.00193818 | 0.00025766 | TRUE | 3510 | 2.1614358590195226 | [0.9515100261527163, |
| | | | | | | 0.8007217559204189] |
| 98 | 0.00205031 | 0.00023638 | TRUE | 3486 | 0.2 | [0.8552427569734735, |
| | | | | | | 0.90477635681313] |
| 99 | 0.00189446 | 0.0002595 | TRUE | 5478 | 1.4403195640356985 | [0.9581154826952613, |
| | | | | | | 0.8403158440361439] |
| 100 | 0.00096915 | 1.2610473443878312e−05 | FALSE | 4898 | 0.6736280026213268 | [0.9385778635807318, |
| | | | | | | 0.9997696800436455] |
| 101 | 0.00146796 | 0.0003315 | TRUE | 3838 | 0.2854799717657287 | [0.8015268790753343, |
| | | | | | | 0.9569646558071104] |
| 102 | 0.00091334 | 0.00024404 | TRUE | 4282 | 2.637714189438057 | [0.9374570388791174, |
| | | | | | | 0.8260910667933302] |
| 103 | 0.00169691 | 0.00034283 | TRUE | 2370 | 5 | [0.936356880485175, |
| | | | | | | 0.8000000000000002] |
| 104 | 0.00151391 | 0.00028382 | TRUE | 7163 | 0.5133362185442419 | [0.938452833649133, |
| | | | | | | 0.9213167592239322] |
| 105 | 0.00224279 | 0.00025252 | TRUE | 5210 | 0.2 | [0.8511949917816688, |
| | | | | | | 0.983963197690045] |
| 106 | 0.00220672 | 0.00018507 | TRUE | 5294 | 1.4920736606114415 | [0.9430985004744069, |
| | | | | | | 0.8444121138452809] |
| 107 | 0.00192575 | 0.0002444 | TRUE | 4515 | 2.372166536942165 | [0.9914192629350086, |
| | | | | | | 0.8000027501202792] |
| 108 | 0.00157582 | 0.00034116 | TRUE | 2219 | 0.2534868039495842 | [0.816253986717725, |
| | | | | | | 0.9540411247461046] |
| 109 | 0.00213569 | 0.0001796 | TRUE | 3134 | 4.1748873537877405 | [0.938231266432264, |
| | | | | | | 0.8010484015586916] |
| 110 | 0.0001 | 0.001 | TRUE | 60138 | 5 | [0.8000000000000002, |
| | | | | | | 0.9550084940763793] |
| 111 | 0.00209527 | 0.00022652 | TRUE | 2052 | 3.912706524172416 | [0.9359647636913262, |
| | | | | | | 0.8032610660230153] |
| 112 | 0.00234988 | 0.0002297 | TRUE | 3642 | 0.2 | [0.8588889784003445, |
| | | | | | | 0.9311061270646593] |
| 113 | 0.00071651 | 5.928145553232412e−05 | TRUE | 21190 | 1.1460241943869756 | [0.8922516775446651, |
| | | | | | | 0.9384048227325351] |
| 114 | 0.00192421 | 0.00028807 | TRUE | 3567 | 1.9343362014598044 | [0.9563113579675467, |
| | | | | | | 0.8000000000000002] |
| 115 | 0.00193154 | 0.00023513 | TRUE | 3721 | 0.4075393994328196 | [0.8490262123580098, |
| | | | | | | 0.959045725769643] |
| 116 | 0.00197632 | 0.00023203 | TRUE | 4049 | 0.2 | [0.8488665233250812, |
| | | | | | | 0.9316628345149028] |
| 117 | 0.00184104 | 0.00041553 | TRUE | 5223 | 0.3935535388039304 | [0.8781594195756147, |
| | | | | | | 0.8543887097641896] |
| 118 | 0.00166754 | 0.00026045 | TRUE | 2426 | 0.379285949 | [0.8623265620912255, |
| | | | | | | 0.969465846964484] |
| 119 | 0.00173868 | 4.817623612345993e−05 | TRUE | 2629 | 4.483917027642813 | [0.9367873241579399, |
| | | | | | | 0.8000000000000002] |
| 120 | 0.00105131 | 1.8968308120566413e−05 | TRUE | 7227 | 0.4204858274956163 | [0.8589627828697415, |
| | | | | | | 0.9665471753595335] |
| 121 | 0.00179401 | 0.00021507 | TRUE | 10079 | 3.428664435206284 | [0.966188615059257, |
| | | | | | | 0.9999] |
| 122 | 0.00179088 | 0.00023427 | TRUE | 10086 | 0.6231897175073273 | [0.9998964709320668, |
| | | | | | | 0.8381787876596429] |
| 123 | 0.00192247 | 0.00022138 | TRUE | 2189 | 1.9025047805520876 | [0.9556837781261733, |
| | | | | | | 0.8000000000000002] |
| 124 | 0.00182312 | 0.00026969 | TRUE | 4131 | 2.2844864765918085 | [0.9567367171930193, |
| | | | | | | 0.8019904680789671] |
| 125 | 0.0022158 | 0.00023641 | TRUE | 4491 | 0.2 | [0.817732073042486, |
| | | | | | | 0.9216872952416935] |
| 126 | 0.0024737 | 0.00024482 | TRUE | 3658 | 0.2523368429339937 | [0.8553820208142129, |
| | | | | | | 0.9998985221657056] |
| 127 | 0.00244727 | 0.00020251 | TRUE | 2048 | 0.2657648175965939 | [0.8000000000000002, |
| | | | | | | 0.9024025990997528] |
| 128 | 0.00157575 | 0.00021371 | TRUE | 2932 | 1.8238683760142944 | [0.9519869172189895, |
| | | | | | | 0.8003881806849538] |
| 129 | 0.00099636 | 0.00017089 | TRUE | 2397 | 3.625102142643079 | [0.932253483721798, |
| | | | | | | 0.8001752358819146] |
| 130 | 0.00074056 | 2.2305433719239025e−05 | FALSE | 15919 | 0.6719894937387761 | [0.8790452619412308, |
| | | | | | | 0.923281196326954] |
| 131 | 0.00185413 | 0.00024524 | TRUE | 2962 | 2.1979601648452083 | [0.952199686355158, |
| | | | | | | 0.8002071950752861] |
| 132 | 0.00189483 | 0.00019368 | TRUE | 5536 | 1.2090097605390897 | [0.9419966586488273, |
| | | | | | | 0.8520890144537906] |
| 133 | 0.00250107 | 0.0002191 | TRUE | 3871 | 0.582982824 | [0.8375977415421102, |
| | | | | | | 0.995685923164099] |
| 134 | 0.00183986 | 0.00018902 | TRUE | 3931 | 2.918770391526907 | [0.9493826837091317, |
| | | | | | | 0.8000305122903533] |
| 135 | 0.00133622 | 0.00087515 | TRUE | 8401 | 0.653657874 | [0.8753157262983575, |
| | | | | | | 0.8551466159649364] |
| 136 | 0.00120301 | 0.00030775 | FALSE | 3925 | 0.9510306227051076 | [0.8849769905595751, |
| | | | | | | 0.8734210226489965] |
| 137 | 0.00902915 | 0.00099175 | FALSE | 15998 | 0.2786162893416954 | [0.9346144727489335, |
| | | | | | | 0.8000000000000002] |
| 138 | 0.00160133 | 0.00098377 | TRUE | 5828 | 4.778810598638802 | [0.8005898366155124, |
| | | | | | | 0.8176915467946264] |
| 139 | 0.00136156 | 0.00099832 | TRUE | 2050 | 1.703347144907556 | [0.8173009371201436, |
| | | | | | | 0.9206072751814451] |
| 140 | 0.00085532 | 0.00097845 | TRUE | 6329 | 0.2526332734200429 | [0.8196781206890653, |
| | | | | | | 0.853964663686142] |
| 141 | 0.00198862 | 0.00031137 | TRUE | 4126 | 0.2002112315716494 | [0.8572346679382056, |
| | | | | | | 0.8930550037613786] |
| 142 | 0.00191599 | 0.00018626 | TRUE | 2616 | 1.9027465848565817 | [0.9471956153194435, |
| | | | | | | 0.8004417667057989] |
| 143 | 0.00113179 | 0.00097455 | TRUE | 4846 | 0.6497049585798531 | [0.8507872912290662, |
| | | | | | | 0.8010094521751874] |
| 144 | 0.00212099 | 0.00024595 | TRUE | 3315 | 0.2 | [0.8302227338503188, |
| | | | | | | 0.8653060349118264] |
| 145 | 0.00181166 | 0.00027645 | TRUE | 4189 | 0.418962063 | [0.8480114234275768, |
| | | | | | | 0.9410158862115604] |
| 146 | 0.00095668 | 9.53531420090476e−05 | FALSE | 40694 | 0.8775626061957631 | [0.8196187384253871, |
| | | | | | | 0.9588572094485014] |
| 147 | 0.00195142 | 0.00035828 | TRUE | 3218 | 0.2 | [0.8314404531969308, |
| | | | | | | 0.8990600500697233] |
| 148 | 0.00118245 | 0.0009478 | TRUE | 4367 | 1.922509659 | [0.8000000000000002, |
| | | | | | | 0.8126800543321062] |
| 149 | 0.00102311 | 4.249484891267758e−05 | FALSE | 5768 | 2.481682184778312 | [0.9472724911835012, |
| | | | | | | 0.9153123774384327] |
| 150 | 0.00149046 | 0.00020872 | TRUE | 6179 | 0.9674994223339436 | [0.9469291396325041, |
| | | | | | | 0.8898974841209237] |
| 151 | 0.00214548 | 0.00022157 | TRUE | 3955 | 1.0125178097000995 | [0.9489169798615851, |
| | | | | | | 0.8470730400898544] |
| 152 | 0.00990588 | 0.00026935 | TRUE | 2939 | 0.356411349 | [0.8000000000000002, |
| | | | | | | 0.8083038451723954] |
| 153 | 0.00326854 | 0.00027521 | TRUE | 3923 | 2.794023666178441 | [0.9510332730068295, |
| | | | | | | 0.844652918807292] |
| 154 | 0.00997599 | 1.0105807207870293e−05 | TRUE | 2066 | 0.7734578548549064 | [0.8000000000000002, |
| | | | | | | 0.8580910391201705] |
| 155 | 0.01 | 0.00034867 | TRUE | 2048 | 0.8492163109763009 | [0.8075336740191639, |
| | | | | | | 0.8209239519801791] |
| 156 | 0.00104256 | 1.8299723788908377e−05 | FALSE | 2058 | 1.0439759855559598 | [0.9788251342235514, |
| | | | | | | 0.9993931152684032] |
| 157 | 0.00214707 | 0.00022687 | TRUE | 3327 | 0.2078399911433376 | [0.8524275386327355, |
| | | | | | | 0.9666242499024097] |
| 158 | 0.00170969 | 6.785443225637707e−05 | TRUE | 5168 | 0.8136763282807475 | [0.8800988447081215, |
| | | | | | | 0.886384916644419] |
| 159 | 0.00187999 | 0.00022162 | TRUE | 2060 | 2.4401257536479664 | [0.9588813980074112, |
| | | | | | | 0.8032651671047636] |
| 160 | 0.00617643 | 0.0009832 | FALSE | 19873 | 0.3538584471778903 | [0.9211189216644516, |
| | | | | | | 0.8009316161271927] |
| 161 | 0.00119541 | 0.0005579 | TRUE | 6752 | 0.2001363651604012 | [0.8000000000000002, |
| | | | | | | 0.8388914483777157] |
| 162 | 0.01 | 0.000152 | TRUE | 2692 | 0.205149399 | [0.8000015551412387, |
| | | | | | | 0.8340958932226975] |
| 163 | 0.00213201 | 0.00023129 | TRUE | 2994 | 0.2341434339809353 | [0.8168503422504392, |
| | | | | | | 0.9714736377117587] |
| 164 | 0.00207262 | 0.00022649 | TRUE | 3648 | 0.2 | [0.8487215832873992, |
| | | | | | | 0.9015522191559018] |
| 165 | 0.00153955 | 0.00031999 | TRUE | 2122 | 0.2946620007203478 | [0.8323096108723481, |
| | | | | | | 0.9498882741794956] |
| 166 | 0.00032808 | 6.53965658913583e−05 | FALSE | 9175 | 1.1029604480036437 | [0.9397045754878226, |
| | | | | | | 0.9370157017664749] |
| 167 | 0.00324048 | 0.00073344 | TRUE | 3867 | 2.029164839618618 | [0.8459071453165166, |
| | | | | | | 0.8389505268680604] |
| 168 | 0.00044363 | 0.00087918 | FALSE | 11109 | 0.8321563475603924 | [0.8000000000000002, |
| | | | | | | 0.8238650650497448] |
| 169 | 0.00445671 | 0.00012903 | FALSE | 30584 | 2.8887923083721763 | [0.8520620863488197, |
| | | | | | | 0.9660183762195138] |
| 170 | 0.00198397 | 0.001 | FALSE | 4363 | 1.4819065958237667 | [0.800754508732578, |
| | | | | | | 0.8769490680626087] |
| 171 | 0.00140759 | 0.00099077 | TRUE | 2049 | 3.5032235872655826 | [0.8005314799544351, |
| | | | | | | 0.8605539287890019] |
| 172 | 0.00149717 | 0.00099747 | TRUE | 3853 | 1.671448905 | [0.8058578976876403, |
| | | | | | | 0.8601183164377151] |
| 173 | 0.00200558 | 0.0001938 | TRUE | 2639 | 2.3276060897632878 | [0.9481107699646132, |
| | | | | | | 0.8000000000000002] |
| 174 | 0.00196083 | 0.0001843 | TRUE | 2426 | 2.769809803143423 | [0.9488200435733002, |
| | | | | | | 0.8001436731904735] |
| 175 | 0.00223353 | 0.00022965 | TRUE | 3329 | 0.2 | [0.8442409731225812, |
| | | | | | | 0.9327788640568453] |
| 176 | 0.00189899 | 0.00025911 | TRUE | 3429 | 2.9615063179802723 | [0.9419300846176939, |
| | | | | | | 0.8031869140966319] |
| 177 | 0.00231341 | 0.00025421 | TRUE | 2906 | 0.310966961 | [0.8527180257551268, |
| | | | | | | 0.9999] |
| 178 | 0.00178569 | 0.00020455 | TRUE | 4963 | 0.8210917052013724 | [0.9467314787323224, |
| | | | | | | 0.8591820965113482] |
| 179 | 0.0013378 | 0.00020283 | TRUE | 2573 | 0.7720271596749725 | [0.9468102349126484, |
| | | | | | | 0.9273098618983352] |
| 180 | 0.0019911 | 0.00019075 | TRUE | 2050 | 3.857454418390027 | [0.9326403155225229, |
| | | | | | | 0.8062982666012967] |
| 181 | 0.00126013 | 0.00020436 | TRUE | 9895 | 1.716152045267386 | [0.9232042941793208, |
| | | | | | | 0.90293909564695] |
| 182 | 0.00093126 | 0.00017315 | TRUE | 24271 | 0.8212950229922447 | [0.8719966505120729, |
| | | | | | | 0.977471848435329] |
| 183 | 0.00213077 | 0.00021551 | TRUE | 2277 | 0.2 | [0.8897411209984485, |
| | | | | | | 0.9277697037178605] |
| 184 | 0.00213032 | 0.00021639 | TRUE | 2096 | 1.8661368030405008 | [0.9482090979359388, |
| | | | | | | 0.8770976908825284] |
| 185 | 0.00164626 | 0.00032179 | TRUE | 4001 | 3.975152280355255 | [0.9591117118248703, |
| | | | | | | 0.8578811574865394] |
| 186 | 0.002087 | 0.0002056 | TRUE | 3427 | 0.2 | [0.8561078822829585, |
| | | | | | | 0.8971969585853388] |
| 187 | 0.00997231 | 1.0825268632211208e−05 | FALSE | 10286 | 0.7806665694223998 | [0.9761511127614714, |
| | | | | | | 0.8003091760642806] |
| 188 | 0.00165906 | 0.00010757 | TRUE | 3212 | 0.2 | [0.8627698686295936, |
| | | | | | | 0.9549410628649656] |
| 189 | 0.00200088 | 0.00017748 | TRUE | 3343 | 2.613624263397274 | [0.943757626033547, |
| | | | | | | 0.8000402771209307] |
| 190 | 0.00109402 | 0.00012131 | FALSE | 6522 | 0.7105536071473617 | [0.9252429410189927, |
| | | | | | | 0.8017909236520193] |
| 191 | 0.01 | 1e−05 | TRUE | 2075 | 0.301933507 | [0.8000000000000002, |
| | | | | | | 0.9228596147746587] |
| 192 | 0.00168697 | 0.00023801 | TRUE | 2995 | 2.229685940625433 | [0.9549461980298101, |
| | | | | | | 0.8060208247639614] |
| 193 | 0.00058538 | 4.28669407768576e−05 | FALSE | 2048 | 1.0707570980446846 | [0.9709696909015356, |
| | | | | | | 0.9996755039254026] |
| 194 | 0.00080864 | 7.788622705872575e−05 | FALSE | 4405 | 0.671019875 | [0.8651706782359306, |
| | | | | | | 0.8895603731384302] |
| 195 | 0.00213798 | 0.00023988 | TRUE | 3184 | 0.2 | [0.9011304570693249, |
| | | | | | | 0.8957463432685372] |
| 196 | 0.00139168 | 3.279330276421203e−05 | FALSE | 10109 | 2.085544934851844 | [0.9370683598293525, |
| | | | | | | 0.9648336793830159] |
| 197 | 0.0011466 | 0.00024933 | TRUE | 2048 | 0.2982237050467003 | [0.8654962449900416, |
| | | | | | | 0.9888546101156531] |
| 198 | 0.00164426 | 0.00017811 | TRUE | 26715 | 3.320320454472375 | [0.9461322850544215, |
| | | | | | | 0.8558630077608341] |
| 199 | 0.00204204 | 0.00012981 | TRUE | 20840 | 0.9021856216303494 | [0.9300911875072968, |
| | | | | | | 0.8685684037638635] |
| 200 | 0.00182854 | 0.0002187 | TRUE | 6295 | 0.8273155900307304 | [0.9571272168217039, |
| | | | | | | 0.8477298549602592] |
| 201 | 0.00211759 | 0.00016677 | TRUE | 5720 | 0.7910716961163551 | [0.9425887980819002, |
| | | | | | | 0.8416257707681646] |
| 202 | 0.00295054 | 0.00019983 | FALSE | 7535 | 1.251624387116013 | [0.8718236867268194, |
| | | | | | | 0.9630163891354875] |
| 203 | 0.00136216 | 1.307298659023908e−05 | TRUE | 2401 | 2.452572082350289 | [0.9933551652678898, |
| | | | | | | 0.8791532903151079] |
| 204 | 0.00996533 | 8.239426299434142e−05 | TRUE | 2053 | 0.9578343686274394 | [0.8000000000000002, |
| | | | | | | 0.8590510546477249] |
| 205 | 0.00188528 | 0.00025722 | TRUE | 3868 | 2.0871727627232155 | [0.9819547554533603, |
| | | | | | | 0.8041586096947009] |
| 206 | 0.00221024 | 0.00023505 | TRUE | 3341 | 0.2 | [0.8000000000000002, |
| | | | | | | 0.9531967694537232] |
| 207 | 0.00078745 | 0.00010924 | TRUE | 10556 | 0.7651920510994988 | [0.8861885563621766, |
| | | | | | | 0.9400321521276552] |
| 208 | 0.00156585 | 0.00041806 | TRUE | 3461 | 0.7599842631040458 | [0.9361352066275413, |
| | | | | | | 0.8500280450393388] |
| 209 | 0.00221961 | 0.00024059 | TRUE | 3824 | 0.2 | [0.8535012021694187, |
| | | | | | | 0.9522027871846404] |
| 210 | 0.00155625 | 0.00096632 | TRUE | 28465 | 1.769287066936219 | [0.8163499862491364, |
| | | | | | | 0.9241777667311157] |
| 211 | 0.0004407 | 7.387646127284622e−05 | FALSE | 13632 | 1.1509856696691687 | [0.8964711424878258, |
| | | | | | | 0.9151853565542438] |
| 212 | 0.00191653 | 0.00019935 | TRUE | 2048 | 4.304504630808991 | [0.9524240743551221, |
| | | | | | | 0.8000000000000002] |
| 213 | 0.000555 | 0.00013751 | TRUE | 6226 | 0.6642935094860098 | [0.9469568786542346, |
| | | | | | | 0.8007666019158524] |
| 214 | 0.00224209 | 0.00025436 | TRUE | 4245 | 0.2 | [0.8340614822177741, |
| | | | | | | 0.9310723010649382] |
| 215 | 0.00104611 | 8.959583527646822e−05 | FALSE | 11244 | 0.9891481510732096 | [0.847598100844541, |
| | | | | | | 0.8999393011490633] |
| 216 | 0.0011859 | 3.579276149699725e−05 | FALSE | 3566 | 3.933467721807022 | [0.9318663651596354, |
| | | | | | | 0.9976073220100004] |
| 217 | 0.00206781 | 0.00017285 | TRUE | 2048 | 4.622397193 | [0.9347420579444129, |
| | | | | | | 0.8000000000000002] |
| 218 | 0.00224005 | 0.00023998 | TRUE | 4046 | 0.2337242326775332 | [0.8495921104014195, |
| | | | | | | 0.931880666129576] |
| 219 | 0.00212477 | 0.00024115 | TRUE | 4156 | 0.2 | [0.845594952256873, |
| | | | | | | 0.9368818397470198] |
| 220 | 0.00209077 | 0.00024567 | TRUE | 3857 | 0.3105151289423642 | [0.848951933999361, |
| | | | | | | 0.9474366698854051] |
| 221 | 0.00178028 | 0.0002137 | TRUE | 4855 | 1.5731030461805189 | [0.9367984872791856, |
| | | | | | | 0.8593722635935144] |
| 222 | 0.00206841 | 0.0002411 | TRUE | 4298 | 0.2 | [0.8000000000000002, |
| | | | | | | 0.9454765677645698] |
| 223 | 0.00240842 | 0.00048744 | FALSE | 9642 | 0.5946045395146358 | [0.8954583718711475, |
| | | | | | | 0.8334351335143892] |
| 224 | 0.0017895 | 0.00031267 | TRUE | 4305 | 0.2 | [0.8426856128939524, |
| | | | | | | 0.9588300888231509] |
| 225 | 0.0019676 | 0.00031134 | FALSE | 3749 | 0.4377686045303768 | [0.8998658489430568, |
| | | | | | | 0.859732819765515] |
| 226 | 0.00527696 | 6.382211079671948e−05 | FALSE | 14195 | 1.291183058466122 | [0.8847055317646872, |
| | | | | | | 0.9704249891517648] |
| 227 | 0.00168625 | 0.00098926 | TRUE | 8896 | 0.794810698 | [0.8973033307244034, |
| | | | | | | 0.8000378767440072] |
| 228 | 0.00209362 | 0.0001891 | TRUE | 2620 | 3.013890142999793 | [0.9434195389051188, |
| | | | | | | 0.8019727434704593] |
| 229 | 0.00114353 | 0.00095861 | FALSE | 7118 | 0.7192169120584965 | [0.8000000000000002, |
| | | | | | | 0.8816863371163384] |
| 230 | 0.00382723 | 0.00031691 | FALSE | 12320 | 1.165547722423343 | [0.9360748512787486, |
| | | | | | | 0.8389816317035557] |
| 231 | 0.00249936 | 0.00011115 | TRUE | 2305 | 4.9116642649294056 | [0.958269610413446, |
| | | | | | | 0.818101379555709] |
| 232 | 0.00197463 | 0.00019884 | FALSE | 2802 | 4.218503661564895 | [0.932715893185091, |
| | | | | | | 0.8023785065305442] |
| 233 | 0.00185715 | 0.00027422 | TRUE | 3205 | 2.644022417531636 | [0.9542041035592325, |
| | | | | | | 0.8000053858575608] |
| 234 | 0.00304937 | 0.001 | TRUE | 2055 | 1.6668218779664563 | [0.8000000000000002, |
| | | | | | | 0.9532421543327876] |
| 235 | 0.00189669 | 0.0002319 | TRUE | 3175 | 2.8150086433182744 | [0.9684854226817264, |
| | | | | | | 0.8014817243473892] |
| 236 | 0.00210593 | 0.00018913 | TRUE | 2620 | 4.119004679484906 | [0.9347559417497648, |
| | | | | | | 0.8009710343917052] |
| 237 | 0.00218971 | 0.00026061 | TRUE | 21961 | 0.2057074931808732 | [0.9103716988476861, |
| | | | | | | 0.9643569346202179] |
| 238 | 0.00200974 | 0.00023787 | TRUE | 3792 | 0.2 | [0.8384340355672419, |
| | | | | | | 0.9417419828375422] |
| 239 | 0.00194454 | 0.0002288 | TRUE | 2287 | 1.695837165679794 | [0.978648947092536, |
| | | | | | | 0.8000000000000002] |
| 240 | 0.00178735 | 0.00011473 | TRUE | 5901 | 0.7893897432538889 | [0.9433096773442571, |
| | | | | | | 0.8347934902755982] |
| 241 | 0.00149983 | 0.0001604 | FALSE | 14665 | 0.998716033 | [0.9281513005522495, |
| | | | | | | 0.8003602162609936] |
| 242 | 0.00211822 | 0.00010463 | TRUE | 2466 | 3.552352829476125 | [0.9540169803140534, |
| | | | | | | 0.8091981545036496] |
| 243 | 0.00191293 | 0.00019721 | TRUE | 2717 | 2.467366218258768 | [0.94936976844401, |
| | | | | | | 0.8024872956487182] |
| 244 | 0.00034251 | 0.001 | TRUE | 5329 | 0.6139248364276209 | [0.8001824320495297, |
| | | | | | | 0.9014482709943804] |
| 245 | 0.000906 | 4.13446343921614e−05 | TRUE | 24950 | 0.908382894 | [0.9130676199641594, |
| | | | | | | 0.8854676153217722] |
| 246 | 0.00258826 | 0.00019558 | TRUE | 12975 | 0.645102533 | [0.9449129988175075, |
| | | | | | | 0.8433084019405418] |
| 247 | 0.00548832 | 0.00010432 | TRUE | 31195 | 1.452290489557885 | [0.9427331207560877, |
| | | | | | | 0.8757117277333634] |
| 248 | 0.0019027 | 0.00026765 | TRUE | 4865 | 2.0009780807549995 | [0.967560485097487, |
| | | | | | | 0.8133678873565907] |
| 249 | 0.00248319 | 6.013611373759829e−05 | FALSE | 4933 | 0.3883684218311475 | [0.9426572831811301, |
| | | | | | | 0.8003932392218788] |
| 250 | 0.00567955 | 4.999021122940766e−05 | TRUE | 31403 | 0.2 | [0.8000000000000002, |
| | | | | | | 0.8977449606246674] |
| 251 | 0.00224359 | 0.00025636 | TRUE | 4542 | 1.0360990813473294 | [0.9532950808580262, |
| | | | | | | 0.8928266541241026] |
| 252 | 0.0097612 | 0.00014879 | TRUE | 5125 | 0.2026845033218955 | [0.8000000000000002, |
| | | | | | | 0.8609521439239823] |
| 253 | 0.00221728 | 0.00023868 | TRUE | 4591 | 0.205691515 | [0.8000000000000002, |
| | | | | | | 0.935472976269334] |
| 254 | 0.01 | 1.0247967465322156e−05 | TRUE | 2048 | 0.680612709 | [0.8000000000000002, |
| | | | | | | 0.8054437173405853] |
| 255 | 0.00264363 | 2.5455992132417927e−05 | FALSE | 6275 | 4.5009158977188575 | [0.9534610695289354, |
| | | | | | | 0.903835014772039] |
| 256 | 0.00181654 | 0.00019831 | TRUE | 2056 | 1.721056481 | [0.9567771884275383, |
| | | | | | | 0.8045203013738096] |
| 257 | 0.00183482 | 0.00049944 | FALSE | 2118 | 4.311080876763394 | [0.8014762401889008, |
| | | | | | | 0.949714413869369] |
| 258 | 0.01 | 2.700253611269488e−05 | TRUE | 2048 | 2.038541454009113 | [0.8000000000000002, |
| | | | | | | 0.8260682845268084] |
| 259 | 0.00085368 | 1e−05 | FALSE | 2048 | 2.543782232866928 | [0.87771309206127, |
| | | | | | | 0.9579202134217231] |
| 260 | 0.0020679 | 0.00022815 | TRUE | 4107 | 0.2 | [0.8559352081161625, |
| | | | | | | 0.9536165137711954] |
| 261 | 0.0099448 | 1.0947649883227862e−05 | TRUE | 2051 | 1.7798995157797113 | [0.8000000000000002, |
| | | | | | | 0.8993655989018154] |
| 262 | 0.00210276 | 0.00025623 | TRUE | 4516 | 0.2272478413787645 | [0.8435850453356396, |
| | | | | | | 0.9431220565025004] |
| 263 | 0.00219562 | 0.000122 | TRUE | 2048 | 0.2 | [0.8761942210895387, |
| | | | | | | 0.9389274369231738] |
| 264 | 0.0013111 | 0.00093244 | TRUE | 2842 | 2.3090042386268945 | [0.8000000000000002, |
| | | | | | | 0.9152619738515275] |
| 265 | 0.00356578 | 0.00095828 | TRUE | 2048 | 2.3460756400556644 | [0.801539765292606, |
| | | | | | | 0.8930085056760022] |
| 266 | 0.0080935 | 1e−05 | TRUE | 2075 | 0.8587108335834547 | [0.8021039054762484, |
| | | | | | | 0.8811498811118438] |
| 267 | 0.00205745 | 0.00024182 | TRUE | 3490 | 2.378590314 | [0.9508132047470532, |
| | | | | | | 0.8000986270750118] |
| 268 | 0.00135499 | 0.00030656 | TRUE | 2048 | 0.3086098503419782 | [0.8831847135836257, |
| | | | | | | 0.9999] |
| 269 | 0.00986538 | 2.5705419692429e−05 | TRUE | 8179 | 0.3082929509418508 | [0.8011095615428763, |
| | | | | | | 0.9045942899361124] |
| 270 | 0.00429673 | 7.913661711714932e−05 | TRUE | 4274 | 0.5401191732683704 | [0.8000000000000002, |
| | | | | | | 0.9568802296175773] |
| 271 | 0.00522109 | 0.0003708 | TRUE | 18838 | 1.0562503818063336 | [0.9008570102939278, |
| | | | | | | 0.8810905112909202] |
| 272 | 0.0019972 | 0.00019736 | TRUE | 2679 | 2.1805391390508397 | [0.9484344889225514, |
| | | | | | | 0.8000000000000002] |
| 273 | 0.00987949 | 2.75339378017434e−05 | TRUE | 3256 | 0.5663164606074172 | [0.8003578196359195, |
| | | | | | | 0.9099152581507877] |
| 274 | 0.00140425 | 0.0006694 | TRUE | 5974 | 0.3096604570275235 | [0.8579678508735005, |
| | | | | | | 0.9804133089599352] |
| 275 | 0.00180804 | 0.00012999 | TRUE | 3180 | 0.6286522547401729 | [0.8441193455274515, |
| | | | | | | 0.9770285691742987] |
| 276 | 0.00209704 | 0.00027802 | TRUE | 5142 | 0.8638238327642077 | [0.9580320084786101, |
| | | | | | | 0.8450800949233743] |
| 277 | 0.00128557 | 0.00019168 | TRUE | 2867 | 0.3719868070761263 | [0.9117262138788952, |
| | | | | | | 0.8000000000000002] |
| 278 | 0.00081987 | 2.7820803884752427e−05 | FALSE | 3367 | 1.1513649847848813 | [0.9270689068048653, |
| | | | | | | 0.9609515377237313] |
| 279 | 0.00020216 | 0.00099464 | TRUE | 4264 | 2.063806074426212 | [0.8000000000000002, |
| | | | | | | 0.8515454233900246] |
| 280 | 0.0024542 | 0.00017232 | TRUE | 3139 | 0.2 | [0.8004278521076212, |
| | | | | | | 0.999753293161775] |
| 281 | 0.00191329 | 0.00020632 | TRUE | 2630 | 1.441252007808819 | [0.9493131811597789, |
| | | | | | | 0.8000000000000002] |
| 282 | 0.00118048 | 0.00037092 | TRUE | 6374 | 1.1181808147924694 | [0.9350724427167326, |
| | | | | | | 0.9094154640984596] |
| 283 | 0.00176363 | 0.00029504 | TRUE | 4602 | 0.7342206196447397 | [0.9663192582143875, |
| | | | | | | 0.8582222949332801] |
| 284 | 0.00194873 | 0.00029206 | TRUE | 3260 | 3.496549298189868 | [0.942844074376569, |
| | | | | | | 0.8029914263749531] |
| 285 | 0.00068998 | 9.83467714109348e−05 | FALSE | 5370 | 1.9753511366045189 | [0.8792248926968911, |
| | | | | | | 0.9463836496654117] |
| 286 | 0.00199165 | 0.00016805 | TRUE | 2919 | 1.9406724868392664 | [0.9475518373807311, |
| | | | | | | 0.8000000000000002] |
| 287 | 0.00197126 | 0.00019349 | TRUE | 2322 | 2.438842278729476 | [0.9547256917380261, |
| | | | | | | 0.8020988359007961] |
| 288 | 0.00213448 | 0.00030816 | TRUE | 6726 | 0.8443572268683408 | [0.9559865142682026, |
| | | | | | | 0.8839824188113764] |
| 289 | 0.00156555 | 0.00023874 | TRUE | 2724 | 2.114739618521933 | [0.9239225590443247, |
| | | | | | | 0.9570174211436929] |
| 290 | 0.00207281 | 0.00023436 | TRUE | 3349 | 0.2 | [0.8740053552098368, |
| | | | | | | 0.9046202121359186] |
| 291 | 0.00204396 | 0.00069553 | TRUE | 2492 | 0.2231546529790191 | [0.8223385505392915, |
| | | | | | | 0.9252223527025828] |
| 292 | 0.00268798 | 0.00018744 | TRUE | 4448 | 0.8927350750365483 | [0.9548646662966314, |
| | | | | | | 0.8593591790778645] |
| 293 | 0.0019475 | 0.0001806 | TRUE | 2773 | 4.068236321406869 | [0.9459647176679716, |
| | | | | | | 0.835242849624972] |
| 294 | 0.00999329 | 5.584165272264053e−05 | TRUE | 3911 | 1.520415125153255 | [0.8701720371898268, |
| | | | | | | 0.9760729074323716] |
| 295 | 0.00170277 | 0.00015546 | TRUE | 6301 | 1.121945228430974 | [0.938247789635858, |
| | | | | | | 0.800814196394611] |
| 296 | 0.0022854 | 0.00025253 | TRUE | 4147 | 0.2 | [0.8522370319576985, |
| | | | | | | 0.9216153001702585] |
| 297 | 0.00189029 | 0.00019978 | TRUE | 2694 | 1.921028574965412 | [0.9532359239408881, |
| | | | | | | 0.801831077610076] |
| 298 | 0.00204276 | 0.00024574 | TRUE | 4871 | 0.2000294396287332 | [0.8388726759566878, |
| | | | | | | 0.9188745338781845] |
| 299 | 0.00198215 | 0.00022786 | TRUE | 4144 | 0.2452873981577459 | [0.8507760867897658, |
| | | | | | | 0.9286378132789301] |
| 300 | 0.00311154 | 0.00022369 | TRUE | 5707 | 0.9638008153183328 | [0.9436296527149636, |
| | | | | | | 0.8473835180003249] |
| 301 | 0.00126794 | 0.00025565 | TRUE | 6157 | 1.083850865500296 | [0.9407853376648875, |
| | | | | | | 0.9032456653958714] |
| 302 | 0.00213737 | 0.00023026 | TRUE | 3429 | 0.2 | [0.8461652174970001, |
| | | | | | | 0.9292927228846286] |
| 303 | 0.00201058 | 0.0002436 | TRUE | 4025 | 0.2 | [0.840868560550553, |
| | | | | | | 0.9215754736405412] |
| 304 | 0.00544714 | 0.00020429 | TRUE | 26028 | 2.712968382632664 | [0.880977857392436, |
| | | | | | | 0.8358668823304647] |
| 305 | 0.00176928 | 0.00099899 | TRUE | 8036 | 0.656094093 | [0.8072574904889791, |
| | | | | | | 0.9068490348929005] |
| 306 | 0.01 | 8.243148114735886e−05 | TRUE | 3029 | 0.3618869628060276 | [0.8029189519491164, |
| | | | | | | 0.8552018164175477] |
| 307 | 0.01 | 0.00099253 | TRUE | 7012 | 0.8205253274992828 | [0.8961924588751525, |
| | | | | | | 0.8439587721054291] |
| 308 | 0.00311012 | 0.00099955 | TRUE | 2048 | 0.6883721105184857 | [0.8019746172296329, |
| | | | | | | 0.933031862597213] |
| 309 | 0.00195339 | 0.00025428 | TRUE | 3667 | 2.580274353897934 | [0.9461204975043325, |
| | | | | | | 0.800083990671623] |
| 310 | 0.00177451 | 0.00027533 | TRUE | 8959 | 1.207602793161897 | [0.9473965576775422, |
| | | | | | | 0.8852976216960355] |
| 311 | 0.00313587 | 0.00020199 | FALSE | 12329 | 1.561574406093906 | [0.9267691886317411, |
| | | | | | | 0.8757211808756192] |
| 312 | 0.00189686 | 6.122300563497622e−05 | FALSE | 6395 | 0.9013030894215516 | [0.9619225572352453, |
| | | | | | | 0.8000000000000002] |
| 313 | 0.00185619 | 0.00024888 | TRUE | 4627 | 2.1304155461935688 | [0.9503007984783236, |
| | | | | | | 0.8009575291629945] |
| 314 | 0.00998722 | 1e−05 | TRUE | 2048 | 0.5585516334742174 | [0.8102866080685893, |
| | | | | | | 0.8682600045023553] |
| 315 | 0.00211153 | 0.0002329 | TRUE | 4867 | 0.854382794 | [0.9492899746023192, |
| | | | | | | 0.8607656770224723] |
| 316 | 0.00213123 | 0.00017659 | TRUE | 2751 | 0.2 | [0.8416815810930567, |
| | | | | | | 0.8778210405610088] |
| 317 | 0.01 | 0.001 | TRUE | 17885 | 0.3934321654156649 | [0.8000000000000002, |
| | | | | | | 0.9999] |
| 318 | 0.00213328 | 0.00019451 | TRUE | 2651 | 2.133355295137511 | [0.9439629587890355, |
| | | | | | | 0.802142927451578] |
| 319 | 0.00193017 | 9.276614542637109e−05 | TRUE | 3201 | 2.7043759049396527 | [0.9508798755208017, |
| | | | | | | 0.8199477709578993] |
| 320 | 0.00348025 | 0.00017236 | TRUE | 2053 | 5 | [0.934750681235816, |
| | | | | | | 0.8000463396471866] |
| 321 | 0.00204602 | 0.00013618 | TRUE | 2065 | 1.3693943810380729 | [0.9437514263130031, |
| | | | | | | 0.8371502630794061] |
| 322 | 0.00553474 | 0.00095585 | TRUE | 2562 | 2.7756570712056536 | [0.8025983098659233, |
| | | | | | | 0.8278206219077615] |
| 323 | 0.00192478 | 0.00024838 | TRUE | 3814 | 0.3315919695197215 | [0.8532891548397488, |
| | | | | | | 0.9455461737878545] |
| 324 | 0.00198853 | 0.00025165 | TRUE | 3858 | 2.336578889735605 | [0.9571348477847363, |
| | | | | | | 0.823549302014404] |
| 325 | 0.00142603 | 0.00025157 | TRUE | 6426 | 2.113539566083453 | [0.9550631072108332, |
| | | | | | | 0.8489034382192601] |
| 326 | 0.00086599 | 3.2792979635752144e−05 | FALSE | 2641 | 1.4316513908859507 | [0.908154267548771, |
| | | | | | | 0.8844873062128732] |
| 327 | 0.00167236 | 0.00021205 | TRUE | 2048 | 1.5564816494851572 | [0.9895027910284289, |
| | | | | | | 0.8541667047709355] |
| 328 | 0.01 | 1e−05 | TRUE | 2053 | 0.6622730573241394 | [0.8019713646415404, |
| | | | | | | 0.8569320669960518] |
| 329 | 0.00196246 | 0.00019695 | TRUE | 3177 | 1.9125990639883907 | [0.9523710541097783, |
| | | | | | | 0.8002880884089009] |
| 330 | 0.00216564 | 0.00023192 | TRUE | 2841 | 0.2 | [0.8438477386357656, |
| | | | | | | 0.8000000000000002] |
| 331 | 0.00170025 | 0.00022955 | TRUE | 4338 | 0.3226662132820181 | [0.8579498185019211, |
| | | | | | | 0.9732723618905245] |
| 332 | 0.00211007 | 0.00034563 | TRUE | 3821 | 0.7997360677841746 | [0.9586864411567997, |
| | | | | | | 0.9118629749474708] |
| 333 | 0.00123837 | 0.00024394 | FALSE | 9275 | 1.120347139291063 | [0.9005366343982159, |
| | | | | | | 0.9014271147489153] |
| 334 | 0.00169936 | 0.00022682 | TRUE | 13553 | 0.9354313649391104 | [0.9458275122684849, |
| | | | | | | 0.9068245441701334] |
| 335 | 0.00474937 | 5.250519453416549e−05 | TRUE | 2048 | 0.20115134 | [0.9395914445271552, |
| | | | | | | 0.8054945819603612] |
| 336 | 0.00266219 | 0.00096256 | TRUE | 53589 | 2.560281656371431 | [0.8064007265659967, |
| | | | | | | 0.984388689487496] |
| 337 | 0.00235576 | 0.00027124 | TRUE | 3539 | 0.9165072342632452 | [0.9573034368416345, |
| | | | | | | 0.8908057886027216] |
| 338 | 0.00197496 | 0.00027926 | TRUE | 6843 | 1.1325018736190786 | [0.9505377867589679, |
| | | | | | | 0.8879935809141223] |
| 339 | 0.00090707 | 0.00045871 | FALSE | 52058 | 0.9098292315219464 | [0.8243561523812672, |
| | | | | | | 0.9999] |
| 340 | 0.00177223 | 0.00034492 | TRUE | 6888 | 0.2007728534111474 | [0.9413824759202439, |
| | | | | | | 0.8675805711352893] |
| 341 | 0.00151492 | 0.00033005 | TRUE | 2048 | 0.9645645111575568 | [0.9228602781496725, |
| | | | | | | 0.8164609829015794] |
| 342 | 0.01 | 0.00016985 | TRUE | 2048 | 0.2 | [0.8000000000000002, |
| | | | | | | 0.8000000000000002] |
| 343 | 0.00191853 | 0.00020235 | TRUE | 3038 | 2.001170315 | [0.9517291125108689, |
| | | | | | | 0.8007743153279084] |
| 344 | 0.01 | 1.0088277465327492e−05 | TRUE | 2048 | 1.440224496039605 | [0.8153842925489193, |
| | | | | | | 0.916409010758249] |
| 345 | 0.00193736 | 0.00018659 | TRUE | 3150 | 2.047677641180887 | [0.9448216558339287, |
| | | | | | | 0.813994335996673] |
| 346 | 0.00216395 | 0.0002251 | TRUE | 3309 | 0.2 | [0.8531318960870035, |
| | | | | | | 0.9237697624255783] |
| 347 | 0.00194982 | 0.00024425 | TRUE | 3975 | 0.2 | [0.8595739130601252, |
| | | | | | | 0.9513242681119012] |
| 348 | 0.00500635 | 0.00083886 | TRUE | 24976 | 0.4692515671484629 | [0.902449661777865, |
| | | | | | | 0.8356655558959877] |
| 349 | 0.01 | 9.829840286897574e−05 | TRUE | 12775 | 0.3472676115819989 | [0.803494594112892, |
| | | | | | | 0.9132733990390844] |
| 350 | 0.0019753 | 0.00021383 | TRUE | 2048 | 2.541428996854996 | [0.951619373940146, |
| | | | | | | 0.8000000000000002] |
| 351 | 0.00205658 | 0.00025886 | TRUE | 4646 | 0.866948806 | [0.9601149195757127, |
| | | | | | | 0.8464823425050607] |
| 352 | 0.00152136 | 0.00026948 | TRUE | 6868 | 0.3595995798918193 | [0.993062232526138, |
| | | | | | | 0.9071639841832526] |
| 353 | 0.0045953 | 2.81844373581666e−05 | FALSE | 8625 | 0.7353200805398769 | [0.9385858120598367, |
| | | | | | | 0.800797381515934] |
| 354 | 0.00187886 | 0.0002224 | TRUE | 2048 | 3.0842815633172425 | [0.9609688365039042, |
| | | | | | | 0.8004019709635809] |
| 355 | 0.00148207 | 0.00098927 | FALSE | 5646 | 1.9504774418931417 | [0.8282100962389727, |
| | | | | | | 0.8141532084974004] |
| 356 | 0.00383649 | 0.00098906 | TRUE | 9373 | 0.819427553 | [0.8634532343328837, |
| | | | | | | 0.8505835007197464] |
| 357 | 0.00035486 | 0.00012446 | TRUE | 12959 | 1.1897297641255242 | [0.9165639394170865, |
| | | | | | | 0.8730329984238938] |
| 358 | 0.00211411 | 0.00017128 | TRUE | 2048 | 0.2 | [0.8733658534349091, |
| | | | | | | 0.9131378816005348] |
| 359 | 0.00277116 | 5.0828458210509095e−05 | FALSE | 5943 | 1.2289138094606615 | [0.9076992961333697, |
| | | | | | | 0.801419535437292] |
| 360 | 0.002656 | 0.00029805 | TRUE | 5865 | 0.8534304298471691 | [0.9515061184514934, |
| | | | | | | 0.8567666455300307] |
| 361 | 0.00044389 | 0.00099577 | TRUE | 2048 | 0.5765858134967047 | [0.8102512109137363, |
| | | | | | | 0.9548144880309639] |
| 362 | 0.00218432 | 0.00030519 | TRUE | 3871 | 0.9321002922291056 | [0.9561166743295029, |
| | | | | | | 0.8654864520849275] |
| 363 | 0.00220864 | 0.00021039 | TRUE | 2048 | 3.3155774601716845 | [0.9541727202495393, |
| | | | | | | 0.8000000000000002] |
| 364 | 0.00218486 | 0.00024626 | TRUE | 2608 | 0.2 | [0.8469335964030023, |
| | | | | | | 0.9169343786118365] |
| 365 | 0.00212918 | 0.00023959 | TRUE | 3489 | 0.2 | [0.8474465151124739, |
| | | | | | | 0.9352138338074153] |
| 366 | 0.00195639 | 0.00028072 | TRUE | 2162 | 1.4138065270066866 | [0.9674456380981487, |
| | | | | | | 0.8001456112793104] |
| 367 | 0.00212467 | 0.00024732 | TRUE | 4144 | 0.2 | [0.8225086979592381, |
| | | | | | | 0.9369596020921763] |
| 368 | 0.00078818 | 0.001 | FALSE | 6225 | 0.6976153633775148 | [0.8749763653690902, |
| | | | | | | 0.8193976582572732] |
| 369 | 0.00207351 | 3.201662204689214e−05 | TRUE | 9501 | 2.8589822258731235 | [0.9396895779032997, |
| | | | | | | 0.9004009404311469] |
| 370 | 0.00212443 | 0.00023367 | TRUE | 2582 | 3.776479594450775 | [0.9379510182993183, |
| | | | | | | 0.8005454046520041] |
| 371 | 0.00279087 | 0.00015734 | TRUE | 20248 | 1.3410278768173658 | [0.9594796067637764, |
| | | | | | | 0.863541405780337] |
| 372 | 0.0023078 | 0.00023781 | TRUE | 3684 | 0.2653429237775517 | [0.8511553239400121, |
| | | | | | | 0.9661633069461792] |
| 373 | 0.00996793 | 1.0005043395082234e−05 | TRUE | 2048 | 1.05325029 | [0.8000000000000002, |
| | | | | | | 0.8000960076441008] |
| 374 | 0.00202744 | 0.00018664 | TRUE | 12506 | 0.8832164824151815 | [0.9199624735373596, |
| | | | | | | 0.8794207431638301] |
| 375 | 0.01 | 1.1460549484426974e−05 | TRUE | 2048 | 1.341052900917711 | [0.8000000000000002, |
| | | | | | | 0.8798771774273829] |
| 376 | 0.00093436 | 0.00013177 | FALSE | 15709 | 0.924883212 | [0.8955643281013979, |
| | | | | | | 0.9359987672354628] |
| 377 | 0.00334676 | 0.00066539 | TRUE | 7853 | 0.6290878995276129 | [0.8734008477655921, |
| | | | | | | 0.8000000000000002] |
| 378 | 0.00209283 | 0.0002427 | TRUE | 2048 | 0.2 | [0.861087468541516, |
| | | | | | | 0.9086844174787254] |
| 379 | 0.00025054 | 0.00095511 | TRUE | 5113 | 0.9640128140179774 | [0.8000000000000002, |
| | | | | | | 0.8383848589361955] |
| 380 | 0.00351297 | 1.0249480211465788e−05 | TRUE | 3114 | 5 | [0.9878026765987542, |
| | | | | | | 0.9036851458171723] |
| 381 | 0.00197237 | 0.00018085 | TRUE | 3058 | 2.571121611707213 | [0.9456176383168717, |
| | | | | | | 0.8185700978031313] |
| 382 | 0.0013769 | 0.00017924 | TRUE | 3267 | 0.7401545931042667 | [0.993496666552196, |
| | | | | | | 0.9178350070859933] |
| 383 | 0.00206738 | 4.141885941188268e−05 | TRUE | 4753 | 0.8815944340629361 | [0.926579269128204, |
| | | | | | | 0.879631449319179] |
| 384 | 0.00150579 | 0.00015912 | TRUE | 3522 | 3.522292721910105 | [0.9353751533078858, |
| | | | | | | 0.8000000000000002] |
| 385 | 0.00187349 | 0.00021556 | FALSE | 3644 | 0.2 | [0.8000000000000002, |
| | | | | | | 0.9999] |
| 386 | 0.00089321 | 0.00095786 | TRUE | 13454 | 0.6411185206266622 | [0.9310440708755766, |
| | | | | | | 0.8009182167093888] |
| 387 | 0.00197198 | 0.00017527 | TRUE | 5383 | 3.117612125358181 | [0.9516741507461184, |
| | | | | | | 0.8008023315388401] |
| 388 | 0.00276455 | 0.00045689 | FALSE | 8779 | 0.4023014398878742 | [0.9251845588623057, |
| | | | | | | 0.8000415220263171] |
| 389 | 0.00564058 | 7.790921537504328e−05 | FALSE | 16926 | 0.9481451573039446 | [0.8949578331614472, |
| | | | | | | 0.8000000000000002] |
| 390 | 0.00031831 | 0.00015302 | FALSE | 34100 | 1.636530531770929 | [0.8618096144007624, |
| | | | | | | 0.9313438350500556] |
| 391 | 0.00012113 | 0.000208 | TRUE | 14389 | 2.632873401350232 | [0.8515500327436237, |
| | | | | | | 0.9242861513399108] |
| 392 | 0.00153756 | 0.00027 | TRUE | 4088 | 0.2819302790572038 | [0.8635512539225516, |
| | | | | | | 0.9644340299196841] |
| 393 | 0.00185429 | 0.00024422 | TRUE | 4120 | 2.0514677280612696 | [0.9606839290672329, |
| | | | | | | 0.8000484752197727 |
| 394 | 0.00170387 | 0.00040315 | TRUE | 7472 | 0.7430691253970926 | [0.9362151784332212, |
| | | | | | | 0.8627142905776304] |
| 395 | 0.00147973 | 0.00024689 | TRUE | 4230 | 0.9443139425507172 | [0.9102490405960353, |
| | | | | | | 0.8769118894395909] |
| 396 | 0.00454485 | 0.00021339 | TRUE | 3102 | 1.9978112901339968 | [0.899424008769877, |
| | | | | | | 0.9945450964411813] |
| 397 | 0.00222619 | 0.00023085 | TRUE | 2211 | 0.2005010718357147 | [0.8748554927972148, |
| | | | | | | 0.8689302817845463] |
| 398 | 0.00997384 | 0.00035888 | TRUE | 23677 | 0.2881806472759844 | [0.8000000000000002, |
| | | | | | | 0.9632576794524373] |
| 399 | 0.00090244 | 6.922594041699108e−05 | FALSE | 3275 | 0.7286874650937162 | [0.8572960017880037, |
| | | | | | | 0.9486744096803981] |
| 400 | 0.00194973 | 0.00023647 | TRUE | 2048 | 2.2779927354743634 | [0.9548264014959691, |
| | | | | | | 0.8105643320699284] |
| 401 | 0.00207131 | 0.0002031 | TRUE | 2504 | 2.291729261967838 | [0.9484522618887318, |
| | | | | | | 0.8021594653191157] |
| 402 | 0.00224986 | 0.00023311 | TRUE | 12360 | 1.2900843314384212 | [0.9386362011771616, |
| | | | | | | 0.8671541215122143] |
| 403 | 0.00151206 | 0.00012023 | TRUE | 10140 | 1.0769287514288148 | [0.969956713840701, |
| | | | | | | 0.8967942250960211] |
| 404 | 0.01 | 0.00024137 | FALSE | 8423 | 0.9006615142824006 | [0.9102354108409242, |
| | | | | | | 0.8766648362461233] |
| 405 | 0.00117301 | 0.00020838 | TRUE | 6090 | 0.4041206415082796 | [0.9388155361775424, |
| | | | | | | 0.9216847598171473] |
| 406 | 0.00104921 | 0.00022062 | TRUE | 2601 | 2.4189625492074587 | [0.9533526576807068, |
| | | | | | | 0.8000000000000002] |
| 407 | 0.00050408 | 0.001 | TRUE | 2048 | 1.8116867470522116 | [0.8000000000000002, |
| | | | | | | 0.9103787052408868] |
| 408 | 0.00210337 | 0.00018522 | TRUE | 2509 | 3.434613829174925 | [0.9339670306705157, |
| | | | | | | 0.8064726156499465] |
| 409 | 0.00180801 | 0.00021291 | TRUE | 4523 | 1.5444554373085573 | [0.9452367222972062, |
| | | | | | | 0.855327199631144] |
| 410 | 0.00193491 | 0.00019963 | TRUE | 3512 | 1.5108101458180505 | [0.9532963313781776, |
| | | | | | | 0.8006505529938938] |
| 411 | 0.00308119 | 0.00035521 | TRUE | 8798 | 0.3322549020866303 | [0.9227460384031004, |
| | | | | | | 0.8036000771936201] |
| 412 | 0.00180909 | 6.242779144291292e−05 | FALSE | 10924 | 1.5207887700163087 | [0.9219131882907365, |
| | | | | | | 0.9272567646179813] |
| 413 | 0.00079288 | 0.00093865 | FALSE | 6792 | 1.0682927759483507 | [0.817545984733405, |
| | | | | | | 0.8691170822777115] |
| 414 | 0.00209648 | 0.00023442 | TRUE | 4450 | 0.2415534508819299 | [0.8220726452900962, |
| | | | | | | 0.9281875802361017] |
| 415 | 0.00123667 | 0.00076045 | TRUE | 6682 | 0.6595356953416133 | [0.9647030204995154, |
| | | | | | | 0.916193938273341] |
| 416 | 0.00214207 | 0.00018785 | TRUE | 4305 | 0.9152709216794174 | [0.9466485866324994, |
| | | | | | | 0.8539810867734787] |
| 417 | 0.00212495 | 0.00023502 | TRUE | 4132 | 0.2 | [0.8368478199937681, |
| | | | | | | 0.9239689069322589] |
| 418 | 0.00212402 | 0.00023242 | TRUE | 5145 | 1.3413323858746606 | [0.9536421879673709, |
| | | | | | | 0.8345210943822794] |
| 419 | 0.00201199 | 0.00017981 | TRUE | 2697 | 2.176550098228624 | [0.9482575627823348, |
| | | | | | | 0.8000114773655594] |
| 420 | 0.00206129 | 0.00027258 | TRUE | 5940 | 0.783713495 | [0.8310022866126849, |
| | | | | | | 0.925752994439539] |
| 421 | 0.00218191 | 0.00026235 | TRUE | 6302 | 0.2 | [0.8292022250890175, |
| | | | | | | 0.9499536732229688] |
| 422 | 0.00183936 | 0.00022965 | TRUE | 2309 | 2.7104798687034286 | [0.9537081025945695, |
| | | | | | | 0.8029424209595741] |
| 423 | 0.0019067 | 0.00025266 | TRUE | 3860 | 2.697060598236169 | [0.9494957475849771, |
| | | | | | | 0.8002846451414596] |
| 424 | 0.002248 | 0.00024431 | TRUE | 4169 | 0.243076407 | [0.8537666454825759, |
| | | | | | | 0.9900737085330368] |
| 425 | 0.00307185 | 0.00028612 | TRUE | 17037 | 1.119840659678308 | [0.9044189134462849, |
| | | | | | | 0.8862358786229311] |
| 426 | 0.00225819 | 0.00021351 | TRUE | 3629 | 0.200015728 | [0.8416679905624315, |
| | | | | | | 0.9498474733615869] |
| 427 | 0.00214608 | 0.00024706 | TRUE | 4325 | 0.2 | [0.8513977477643015, |
| | | | | | | 0.9052072487900773] |
| 428 | 0.00037052 | 8.214778486516948e−05 | FALSE | 3882 | 1.0786521926169723 | [0.9289068557524631, |
| | | | | | | 0.8000000000000002] |
| 429 | 0.00192482 | 0.00021774 | TRUE | 3476 | 1.3153691830730645 | [0.95269684701934, |
| | | | | | | 0.8011959803362222] |
| 430 | 0.00089772 | 0.00022121 | FALSE | 11879 | 0.9859994980947604 | [0.9252947304992931, |
| | | | | | | 0.8614238689270021] |
| 431 | 0.00204001 | 0.00019628 | TRUE | 2344 | 2.105845786 | [0.9502534688151835, |
| | | | | | | 0.8000000000000002] |
| 432 | 0.01 | 6.593669578081655e−05 | TRUE | 6924 | 0.3206782655432074 | [0.8595494021352802, |
| | | | | | | 0.8382786285220063] |
| 433 | 0.00324192 | 0.00021132 | FALSE | 3940 | 0.2 | [0.8000000000000002, |
| | | | | | | 0.9998955898118271] |
| 434 | 0.00958176 | 1.0363721130352134e−05 | TRUE | 7745 | 0.396041889 | [0.9999, |
| | | | | | | 0.8000000000000002] |
| 435 | 0.00389223 | 0.00099553 | TRUE | 3208 | 1.261858495617807 | [0.8292881643843719, |
| | | | | | | 0.8051872239236079] |
| 436 | 0.00197451 | 0.00024083 | TRUE | 3492 | 2.412576933173653 | [0.9510118838241657, |
| | | | | | | 0.8026789103035026] |
| 437 | 0.00211862 | 0.0002272 | TRUE | 2474 | 0.2 | [0.9695650486415204, |
| | | | | | | 0.9173926376386905] |
| 438 | 0.00209496 | 0.00023735 | TRUE | 4459 | 0.2110678485147896 | [0.8228269659758887, |
| | | | | | | 0.928835910938008] |
| 439 | 0.00093215 | 0.001 | TRUE | 2081 | 4.945007811323316 | [0.8008159041673254 |
| | | | | | | 0.8000000000000002] |
| 440 | 0.00204286 | 0.00023817 | TRUE | 3915 | 0.2 | [0.8343545606749458, |
| | | | | | | 0.943169406883615] |
| 441 | 0.00198322 | 0.00014789 | TRUE | 4294 | 1.6037532117962308 | [0.9444714889663801, |
| | | | | | | 0.8474082978518087] |
| 442 | 0.00239637 | 0.0002438 | TRUE | 2245 | 4.841615712442299 | [0.9347826382210274, |
| | | | | | | 0.8000000000000002] |
| 443 | 0.00223819 | 0.00023022 | TRUE | 3596 | 0.2 | [0.8605239190422296, |
| | | | | | | 0.973547653835692] |
| 444 | 0.01 | 1.9825787855472984e−05 | TRUE | 2119 | 1.202594106978828 | [0.8011590459430878, |
| | | | | | | 0.865933772255555] |
| 445 | 0.00220727 | 0.00021153 | TRUE | 2295 | 0.2 | [0.8926089445202718, |
| | | | | | | 0.923074063779513] |
| 446 | 0.00155611 | 0.00021566 | TRUE | 2755 | 0.2441690849651707 | [0.8548002964096708, |
| | | | | | | 0.9961740946989832] |
| 447 | 0.00191613 | 0.00019899 | TRUE | 2635 | 2.161169522399787 | [0.9558050382432688, |
| | | | | | | 0.8002074179192072] |
| 448 | 0.00186701 | 0.00026133 | TRUE | 3573 | 2.475326172469776 | [0.9449275424717694, |
| | | | | | | 0.8000000000000002] |
| 449 | 0.00184969 | 0.00021466 | TRUE | 4677 | 0.2744882453431891 | [0.8000000000000002, |
| | | | | | | 0.8136265400011842] |
| 450 | 0.00181834 | 0.00027928 | TRUE | 3950 | 2.664484816482072 | [0.9512078572191207, |
| | | | | | | 0.8000000000000002] |
| 451 | 0.01 | 0.00011117 | FALSE | 6268 | 0.232098318 | [0.8016553851081829, |
| | | | | | | 0.8764431175345707] |
| 452 | 0.00195589 | 0.00024658 | TRUE | 4310 | 0.2162647106966547 | [0.8362710853814066, |
| | | | | | | 0.9513662134476776] |
| 453 | 0.00098853 | 5.139940007827596e−05 | FALSE | 2820 | 1.4452066648620217 | [0.8370121552574149, |
| | | | | | | 0.8948073789661728] |
| 454 | 0.00242207 | 0.00022994 | TRUE | 3864 | 0.225206743 | [0.8467810883698368, |
| | | | | | | 0.9795646992033349] |
| 455 | 0.00177798 | 0.00018898 | TRUE | 4937 | 1.8483974314618437 | [0.9442042980506731, |
| | | | | | | 0.8565322817552552] |
| 456 | 0.00219823 | 0.000339 | TRUE | 7593 | 0.2005298073119752 | [0.8225991366729839, |
| | | | | | | 0.906149518166058] |
| 457 | 0.00042678 | 4.5489151836610726e−05 | TRUE | 6392 | 1.0184938146880096 | [0.9192624049747429, |
| | | | | | | 0.9079493547797112] |
| 458 | 0.00219993 | 0.00025294 | TRUE | 3918 | 0.203906942 | [0.879834569379536, |
| | | | | | | 0.8952964764159913] |
| 459 | 0.00194763 | 0.00021091 | TRUE | 3743 | 1.849864670483302 | [0.9592016803326041, |
| | | | | | | 0.8000000000000002] |
| 460 | 0.00267174 | 0.001 | TRUE | 4002 | 1.6813068784185283 | [0.860765121987508, |
| | | | | | | 0.8344196329390046] |
| 461 | 0.00231587 | 0.00023393 | TRUE | 3491 | 0.259583956 | [0.82954965469096, |
| | | | | | | 0.9579644829733183] |
| 462 | 0.00214384 | 0.00024088 | TRUE | 3196 | 0.2089107864244288 | [0.8426393482579355, |
| | | | | | | 0.9022416541288876] |
| 463 | 0.0014862 | 0.00021532 | TRUE | 5225 | 1.130880286 | [0.935331765904192, |
| | | | | | | 0.9653247267085816] |
| 464 | 0.00196418 | 0.00014093 | TRUE | 5114 | 0.6599820887494174 | [0.9648488421855856, |
| | | | | | | 0.8201275276639831] |
| 465 | 0.00177118 | 0.00022087 | TRUE | 3456 | 0.8656219614021254 | [0.9351671941799231, |
| | | | | | | 0.9362284698033626] |
| 466 | 0.00165637 | 4.170509801512538e−05 | TRUE | 4098 | 2.050635016719212 | [0.8583290039415187, |
| | | | | | | 0.9126662005758823] |
| 467 | 0.00193684 | 0.00016758 | TRUE | 2048 | 3.950923522351877 | [0.9475973205073611, |
| | | | | | | 0.8000000000000002] |
| 468 | 0.00191228 | 0.00025773 | TRUE | 3044 | 0.2952226881381748 | [0.8644809142273967, |
| | | | | | | 0.9810884806457834] |
| 469 | 0.0012824 | 0.00016198 | FALSE | 13401 | 1.0116123007679407 | [0.913926050802429, |
| | | | | | | 0.8508796497997471] |
| 470 | 0.00253504 | 0.00024673 | TRUE | 11866 | 0.9079618907810012 | [0.9400293657561112, |
| | | | | | | 0.9057667840860519] |
| 471 | 0.00202358 | 0.00018917 | TRUE | 2818 | 3.7116708178578857 | [0.9206343753817604, |
| | | | | | | 0.8006962028440838] |
| 472 | 0.00167891 | 0.00018233 | TRUE | 8907 | 0.8943761603361069 | [0.9413389189678104, |
| | | | | | | 0.980885657053606] |
| 473 | 0.0020345 | 0.00058705 | TRUE | 5650 | 0.5497449586396926 | [0.9328177162041192, |
| | | | | | | 0.8538284984016307] |
| 474 | 0.00216537 | 0.00023929 | TRUE | 2726 | 0.2 | [0.8468336431468741, |
| | | | | | | 0.910579195783829] |
| 475 | 0.00209408 | 4.938141678471807e−05 | TRUE | 2322 | 4.795397779251042 | [0.9371107170101927, |
| | | | | | | 0.8000000000000002] |
| 476 | 0.00136449 | 0.00021616 | FALSE | 10097 | 1.019045791054079 | [0.9105757443697251, |
| | | | | | | 0.8890088258811207] |
| 477 | 0.00170797 | 0.00025778 | TRUE | 2048 | 0.2621329602379211 | [0.8459882907810261, |
| | | | | | | 0.9331749146625906] |
| 478 | 0.0017108 | 0.00017718 | TRUE | 2239 | 0.9669689498198256 | [0.9954513369671125, |
| | | | | | | 0.9804923821529372] |
| 479 | 0.00058262 | 7.348201222861329e−05 | FALSE | 7189 | 1.536505112393235 | [0.9045164845897729, |
| | | | | | | 0.9466627242139578] |
| 480 | 0.0013433 | 0.00031157 | TRUE | 2048 | 0.2 | [0.8766246473525467, |
| | | | | | | 0.9789433494262156] |
| 481 | 0.00401261 | 0.0009023 | TRUE | 7668 | 1.136436896367475 | [0.8986444592413075, |
| | | | | | | 0.9346875380535185] |
| 482 | 0.01 | 2.586636343594856e−05 | TRUE | 2075 | 0.8077813523704749 | [0.8000000000000002, |
| | | | | | | 0.8755435922571194] |
| 483 | 0.00187039 | 0.00025534 | TRUE | 2154 | 4.725038783998376 | [0.9583231839592784, |
| | | | | | | 0.8000000000000002] |
| 484 | 0.01 | 1e−05 | TRUE | 2048 | 0.2 | [0.8049815119834016, |
| | | | | | | 0.8760627808044873] |
| 485 | 0.00160627 | 0.00023492 | TRUE | 5578 | 0.3926961182521655 | [0.9346410449278106, |
| | | | | | | 0.9462039740175736] |
| 486 | 0.00194869 | 0.00022531 | TRUE | 4488 | 1.0343336393301492 | [0.943707928736631, |
| | | | | | | 0.952005181434806] |
| 487 | 0.00087356 | 0.00070209 | TRUE | 6684 | 0.8736472463549945 | [0.8270614291929191, |
| | | | | | | 0.8441066829584032] |
| 488 | 0.01 | 1e−05 | TRUE | 2276 | 1.1790315019132274 | [0.8000000000000002, |
| | | | | | | 0.8027456395659724] |
| 489 | 0.00183855 | 0.00018549 | TRUE | 3926 | 0.6092945593885375 | [0.9245613118842732, |
| | | | | | | 0.9121686837816303] |
| 490 | 0.00184107 | 0.00020431 | TRUE | 2766 | 3.775743708944719 | [0.9543213181791081, |
| | | | | | | 0.8051471788487738] |
| 491 | 0.00141344 | 0.00091747 | TRUE | 2853 | 0.5939574330576871 | [0.9644156021786713, |
| | | | | | | 0.980884923254181] |
| 492 | 0.00174064 | 0.00079722 | TRUE | 2052 | 0.2 | [0.8313102065831719, |
| | | | | | | 0.9666205671088738] |

| Row ID | timestamp |
|---|---|
| 1 | 20231230_234615 |
| 2 | 20240101_182906 |
| 3 | 20240104_183427 |
| 4 | 20231231_030342 |
| 5 | 20240105_031736 |
| 6 | 20240101_133621 |
| 7 | 20240102_033238 |
| 8 | 20240102_191530 |
| 9 | 20240102_145238 |
| 10 | 20240105_084540 |
| 11 | 20240104_091336 |
| 12 | 20240104_030146 |
| 13 | 20231231_034400 |
| 14 | 20240102_104635 |
| 15 | 20231231_214937 |
| 16 | 20240103_125049 |
| 17 | 20240102_195621 |
| 18 | 20231231_010734 |
| 19 | 20240103_202813 |
| 20 | 20240105_074323 |
| 21 | 20240102_064347 |
| 22 | 20240103_094751 |
| 23 | 20240104_200948 |
| 24 | 20231231_155355 |
| 25 | 20240102_011026 |
| 26 | 20231231_044616 |
| 27 | 20231231_015401 |
| 28 | 20240101_104406 |
| 29 | 20231231_035919 |
| 30 | 20231231_151318 |
| 31 | 20240101_152717 |
| 32 | 20240104_020527 |
| 33 | 20231231_005103 |
| 34 | 20240101_101838 |
| 35 | 20240101_185040 |
| 36 | 20240103_032950 |
| 37 | 20240103_005745 |
| 38 | 20240104_175927 |
| 39 | 20240101_000711 |
| 40 | 20231230_235149 |
| 41 | 20240106_132306 |
| 42 | 20240103_195331 |
| 43 | 20240106_044832 |
| 44 | 20240106_024527 |
| 45 | 20231230_194028 |
| 46 | 20240101_155820 |
| 47 | 20240101_013931 |
| 48 | 20231231_061941 |
| 49 | 20240101_200726 |
| 50 | 20231231_074821 |
| 51 | 20240105_072315 |
| 52 | 20240104_074602 |
| 53 | 20240102_155106 |
| 54 | 20240101_191130 |
| 55 | 20231230_223326 |
| 56 | 20240104_070204 |
| 57 | 20240101_151416 |
| 58 | 20240105_100142 |
| 59 | 20240104_231328 |
| 60 | 20240103_223031 |
| 61 | 20240101_012658 |
| 62 | 20231230 214427 |
| 63 | 20240104_023942 |
| 64 | 20240106_144850 |
| 65 | 20231231_211251 |
| 66 | 20240101_003424 |
| 67 | 20240102_180457 |
| 68 | 20240101_111958 |
| 69 | 20240103_065959 |
| 70 | 20240102_085502 |
| 71 | 20240103_134415 |
| 72 | 20240102_141410 |
| 73 | 20240103_002126 |
| 74 | 20240102_043532 |
| 75 | 20240106_151914 |
| 76 | 20240102_204631 |
| 77 | 20240102_074852 |
| 78 | 20240104_203343 |
| 79 | 20240103_063719 |
| 80 | 20240102_153629 |
| 81 | 20240104_052441 |
| 82 | 20240103_073917 |
| 83 | 20240105_181318 |
| 84 | 20240103_025024 |
| 85 | 20240106_020127 |
| 86 | 20240101_103351 |
| 87 | 20240106_154257 |
| 88 | 20240104_062919 |
| 89 | 20240102_224754 |
| 90 | 20231230_200830 |
| 91 | 20240101_211812 |
| 92 | 20240102_121706 |
| 93 | 20240103_201748 |
| 94 | 20240102_175207 |
| 95 | 20240103_082132 |
| 96 | 20240105_012828 |
| 97 | 20240103_193005 |
| 98 | 20240104_132711 |
| 99 | 20240102_013915 |
| 100 | 20231231_163856 |
| 101 | 20240101_032628 |
| 102 | 20240103_095634 |
| 103 | 20240103_134215 |
| 104 | 20240101_024903 |
| 105 | 20240102_003013 |
| 106 | 20240102_164347 |
| 107 | 20240105_135908 |
| 108 | 20240101_151304 |
| 109 | 20240103_135719 |
| 110 | 20231230_214456 |
| 111 | 20240103_130752 |
| 112 | 20240103_155541 |
| 113 | 20231231_002104 |
| 114 | 20240105_082019 |
| 115 | 20240101_194918 |
| 116 | 20240102_115314 |
| 117 | 20231231_075537 |
| 118 | 20231231_232840 |
| 119 | 20240103_135114 |
| 120 | 20231230_224110 |
| 121 | 20231231_144608 |
| 122 | 20240101_030716 |
| 123 | 20240105_122227 |
| 124 | 20240104_170553 |
| 125 | 20240102_073129 |
| 126 | 20240102_053911 |
| 127 | 20240101_210138 |
| 128 | 20240104_153521 |
| 129 | 20240103_115245 |
| 130 | 20231230_205440 |
| 131 | 20240104_114955 |
| 132 | 20240102_141705 |
| 133 | 20240101_223129 |
| 134 | 20240103_184824 |
| 135 | 20240103_081903 |
| 136 | 20231231_070858 |
| 137 | 20240102_182916 |
| 138 | 20240105_220205 |
| 139 | 20240106_150709 |
| 140 | 20240104_012417 |
| 141 | 20240104_124411 |
| 142 | 20240105_081648 |
| 143 | 20240104_033520 |
| 144 | 20240105_135326 |
| 145 | 20240103_165241 |
| 146 | 20240102_023316 |
| 147 | 20240106_000416 |
| 148 | 20240105_033500 |
| 149 | 20231231_124410 |
| 150 | 20240101_045508 |
| 151 | 20240101_155156 |
| 152 | 20240103_101719 |
| 153 | 20240103_201849 |
| 154 | 20240106_165741 |
| 155 | 20240104_225204 |
| 156 | 20231231_154633 |
| 157 | 20240101_221606 |
| 158 | 20231231_155727 |
| 159 | 20240105_175428 |
| 160 | 20240102_192951 |
| 161 | 20240104_224040 |
| 162 | 20240102_231157 |
| 163 | 20240101_212804 |
| 164 | 20240105_062103 |
| 165 | 20240101_161225 |
| 166 | 20240101_075225 |
| 167 | 20240105_125159 |
| 168 | 20240104_171606 |
| 169 | 20240102_055029 |
| 170 | 20240106_172202 |
| 171 | 20240106_005740 |
| 172 | 20240106_121235 |
| 173 | 20240104_201809 |
| 174 | 20240105_173729 |
| 175 | 20240104_122102 |
| 176 | 20240103_211035 |
| 177 | 20240102_023123 |
| 178 | 20231231_224700 |
| 179 | 20240101_022715 |
| 180 | 20240103_142954 |
| 181 | 20231231_040345 |
| 182 | 20240101_042050 |
| 183 | 20240106_102735 |
| 184 | 20240103_005252 |
| 185 | 20240102_030955 |
| 186 | 20240105_092548 |
| 187 | 20240101_064513 |
| 188 | 20240101_170821 |
| 189 | 20240103_230832 |
| 190 | 20240102_093658 |
| 191 | 20240106_004053 |
| 192 | 20240104_012046 |
| 193 | 20231231_140254 |
| 194 | 20231231_124846 |
| 195 | 20240105_220719 |
| 196 | 20231231_172303 |
| 197 | 20240101_133649 |
| 198 | 20240102_094901 |
| 199 | 20231231_114359 |
| 200 | 20240101_061249 |
| 201 | 20231231_134656 |
| 202 | 20240102_065852 |
| 203 | 20231231_125506 |
| 204 | 20240104_135647 |
| 205 | 20240105_215955 |
| 206 | 20240102_060205 |
| 207 | 20231231_082507 |
| 208 | 20231231_185744 |
| 209 | 20240101_225324 |
| 210 | 20240104_181052 |
| 211 | 20231230_233811 |
| 212 | 20240103_173721 |
| 213 | 20240103_044025 |
| 214 | 20240103_092329 |
| 215 | 20231230_214216 |
| 216 | 20231231_170612 |
| 217 | 20240103_122110 |
| 218 | 20240103_034425 |
| 219 | 20240102_065754 |
| 220 | 20240101_214521 |
| 221 | 20240102_125016 |
| 222 | 20240102_063213 |
| 223 | 20231230_223056 |
| 224 | 20240101_183703 |
| 225 | 20240104_091022 |
| 226 | 20240101_093546 |
| 227 | 20240104_202408 |
| 228 | 20240104_075612 |
| 229 | 20240104_083437 |
| 230 | 20240102_005949 |
| 231 | 20240103_053257 |
| 232 | 20240103_135306 |
| 233 | 20240104_182813 |
| 234 | 20240106_051433 |
| 235 | 20240103_200335 |
| 236 | 20240103_152438 |
| 237 | 20240103_224858 |
| 238 | 20240102_153412 |
| 239 | 20240106_134915 |
| 240 | 20231231_204448 |
| 241 | 20240102_051445 |
| 242 | 20240103_061039 |
| 243 | 20240105_161057 |
| 244 | 20240104_041531 |
| 245 | 20240101_094716 |
| 246 | 20240102_012328 |
| 247 | 20231231_110114 |
| 248 | 20240105_142950 |
| 249 | 20240103_060521 |
| 250 | 20240102_094539 |
| 251 | 20240101_140830 |
| 252 | 20240102_213416 |
| 253 | 20240102_072634 |
| 254 | 20240106_130038 |
| 255 | 20231230_192458 |
| 256 | 20240106_063717 |
| 257 | 20240106_060405 |
| 258 | 20240105_225809 |
| 259 | 20231231_122633 |
| 260 | 20240101_195658 |
| 261 | 20240106_071703 |
| 262 | 20240102_224506 |
| 263 | 20240106_124120 |
| 264 | 20240106_113359 |
| 265 | 20240106_083022 |
| 266 | 20240105_180825 |
| 267 | 20240104_004656 |
| 268 | 20240101_135727 |
| 269 | 20240103_025336 |
| 270 | 20240102_233803 |
| 271 | 20231231_020328 |
| 272 | 20240104_224257 |
| 273 | 20240103_091616 |
| 274 | 20240101_120603 |
| 275 | 20240101_162544 |
| 276 | 20240101_051557 |
| 277 | 20240102_121006 |
| 278 | 20231231_060349 |
| 279 | 20240105_043505 |
| 280 | 20240102_000822 |
| 281 | 20240104_183427 |
| 282 | 20231231_143920 |
| 283 | 20231231_184032 |
| 284 | 20240104_132855 |
| 285 | 20231231_055300 |
| 286 | 20240106_024450 |
| 287 | 20240105_225921 |
| 288 | 20240101_061100 |
| 289 | 20231231_213203 |
| 290 | 20240104_114356 |
| 291 | 20240106_095605 |
| 292 | 20240101_133005 |
| 293 | 20240102_230319 |
| 294 | 20240102_170049 |
| 295 | 20231231_171732 |
| 296 | 20240103_004733 |
| 297 | 20240105_024556 |
| 298 | 20240102_102938 |
| 299 | 20240102_130017 |
| 300 | 20240102_031703 |
| 301 | 20240101_040849 |
| 302 | 20240104_174140 |
| 303 | 20240102_145045 |
| 304 | 20231231_065020 |
| 305 | 20240104_151632 |
| 306 | 20240103_143229 |
| 307 | 20231231_075008 |
| 308 | 20240105_214817 |
| 309 | 20240103_222333 |
| 310 | 20240101_081045 |
| 311 | 20231230_235320 |
| 312 | 20240103_083115 |
| 313 | 20240105_061415 |
| 314 | 20240105_155722 |
| 315 | 20240101_061218 |
| 316 | 20240104_065131 |
| 317 | 20240102_215322 |
| 318 | 20240104_023224 |
| 319 | 20240103_190445 |
| 320 | 20240103_115038 |
| 321 | 20240102_181957 |
| 322 | 20240105_160603 |
| 323 | 20240101_202119 |
| 324 | 20240103_155440 |
| 325 | 20240103_023148 |
| 326 | 20231231_112948 |
| 327 | 20240101_013918 |
| 328 | 20240105_211623 |
| 329 | 20240104_062632 |
| 330 | 20240105_020851 |
| 331 | 20240101_123647 |
| 332 | 20240101_055336 |
| 333 | 20231231_051859 |
| 334 | 20231231_115320 |
| 335 | 20240103_232414 |
| 336 | 20240105_023106 |
| 337 | 20240101_111954 |
| 338 | 20240102_082516 |
| 339 | 20240101_000637 |
| 340 | 20231231_101700 |
| 341 | 20231231_101118 |
| 342 | 20240103_051842 |
| 343 | 20240105_120841 |
| 344 | 20240106_025419 |
| 345 | 20240104_072221 |
| 346 | 20240104_104330 |
| 347 | 20240102_002834 |
| 348 | 20240104_133611 |
| 349 | 20240102_074445 |
| 350 | 20240104_144445 |
| 351 | 20240101_100200 |
| 352 | 20240101_004551 |
| 353 | 20240101_033317 |
| 354 | 20240104_223220 |
| 355 | 20240105_111511 |
| 356 | 20240105_193512 |
| 357 | 20231231_004348 |
| 358 | 20240106_143230 |
| 359 | 20240103_022108 |
| 360 | 20240101_062833 |
| 361 | 20240103_220336 |
| 362 | 20240101_082120 |
| 363 | 20240103_104836 |
| 364 | 20240106_011227 |
| 365 | 20240105_145153 |
| 366 | 20240102_225110 |
| 367 | 20240103_033837 |
| 368 | 20240104_051237 |
| 369 | 20231230_202605 |
| 370 | 20240103_165022 |
| 371 | 20231230_225235 |
| 372 | 20240102_051419 |
| 373 | 20240106_113017 |
| 374 | 20231230_211618 |
| 375 | 20240105_111014 |
| 376 | 20231231_205318 |
| 377 | 20240104_043258 |
| 378 | 20240105_193739 |
| 379 | 20240105_084512 |
| 380 | 20231230_202319 |
| 381 | 20240103_002019 |
| 382 | 20231231_141329 |
| 383 | 20231231_164621 |
| 384 | 20240103_124835 |
| 385 | 20240103_175049 |
| 386 | 20231231_025701 |
| 387 | 20240103_170016 |
| 388 | 20240102_185719 |
| 389 | 20240102_040234 |
| 390 | 20240101_222512 |
| 391 | 20231230_211025 |
| 392 | 20240101_085801 |
| 393 | 20240105_114053 |
| 394 | 20231231_215444 |
| 395 | 20231231_193236 |
| 396 | 20240102_072632 |
| 397 | 20240106_105426 |
| 398 | 20240102_205508 |
| 399 | 20231231_111754 |
| 400 | 20240102_234452 |
| 401 | 20240104_193930 |
| 402 | 20231231_000719 |
| 403 | 20231231_050831 |
| 404 | 20231231_072158 |
| 405 | 20240101_071918 |
| 406 | 20240104_023521 |
| 407 | 20240106_170259 |
| 408 | 20240104_101531 |
| 409 | 20240102_151834 |
| 410 | 20240106_062017 |
| 411 | 20240102_092932 |
| 412 | 20231230_204819 |
| 413 | 20240103_201940 |
| 414 | 20240102_105000 |
| 415 | 20231231_123342 |
| 416 | 20240101_074907 |
| 417 | 20240102_105310 |
| 418 | 20240101_154735 |
| 419 | 20240104_113317 |
| 420 | 20240102_204115 |
| 421 | 20240102_202602 |
| 422 | 20240105_050030 |
| 423 | 20240103_183833 |
| 424 | 20240101_234452 |
| 425 | 20231231_050514 |
| 426 | 20240104_011641 |
| 427 | 20240105_211639 |
| 428 | 20240103_034449 |
| 429 | 20240106_000326 |
| 430 | 20231231_064244 |
| 431 | 20240106_023248 |
| 432 | 20240102_174334 |
| 433 | 20240103_205916 |
| 434 | 20240101_090926 |
| 435 | 20240106_080240 |
| 436 | 20240105_010130 |
| 437 | 20240106_050101 |
| 438 | 20240102_122929 |
| 439 | 20240105_092851 |
| 440 | 20240103_012142 |
| 441 | 20240103_205155 |
| 442 | 20240103_124740 |
| 443 | 20240101_181324 |
| 444 | 20240103_154647 |
| 445 | 20240106_170539 |
| 446 | 20240101_052810 |
| 447 | 20240105_210530 |
| 448 | 20240103_171620 |
| 449 | 20240101_202444 |
| 450 | 20240104_071417 |
| 451 | 20240102_142015 |
| 452 | 20240102_190512 |
| 453 | 20231231_095810 |
| 454 | 20240102_003612 |
| 455 | 20240102_110054 |
| 456 | 20240103_041000 |
| 457 | 20231230_193800 |
| 458 | 20240102_041513 |
| 459 | 20240106_035300 |
| 460 | 20240106_010525 |
| 461 | 20240102_032435 |
| 462 | 20240105_154823 |
| 463 | 20231231_141852 |
| 464 | 20231231_173925 |
| 465 | 20231231_195445 |
| 466 | 20231231_085552 |
| 467 | 20240103_090531 |
| 468 | 20240101_130929 |
| 469 | 20231231_052258 |
| 470 | 20231231_055111 |
| 471 | 20240106_173052 |
| 472 | 20231231_234511 |
| 473 | 20231231_194408 |
| 474 | 20240105_224719 |
| 475 | 20240103_101419 |
| 476 | 20231231_062954 |
| 477 | 20240101_165134 |
| 478 | 20231231_231518 |
| 479 | 20231230_210925 |
| 480 | 20240101_130848 |
| 481 | 20240104_210429 |
| 482 | 20240105_121628 |
| 483 | 20240103_055422 |
| 484 | 20240106_090107 |
| 485 | 20231231_192451 |
| 486 | 20240101_024342 |
| 487 | 20240104_233244 |
| 488 | 20240105_031052 |
| 489 | 20231231_213957 |
| 490 | 20240103_162308 |
| 491 | 20231231_204537 |
| 492 | 20240101_131206 |

Table Headers:
hepg2_test = test set performance for HepG2;
hepg2_val = validation set performance for HepG2;
sknsh_test = test set performance for SK—N—SH;
sknsh_val = validation set performance for SK—N—SH;
k562_test = test set performance for K562;
k562_val = validation set performance for K562;
batch_size = training loop batch size;
padded_seq_len = total sequence length for model inputs after padding;
duplication_cutoff = minimum activity cutoff for training set duplication;
use_reverse_complements = training data augmentation, train on both forward and reverse complements of padded sequences;
input_len = nput length for model, should match padded_seq_len;
conv1_channels = out_channels for torch.nn.Conv1d at the first layer;
conv1_kernel_size = kernel_size for torch.nn.Conv1d at the first layer;
conv2_channels = out_channels for torch.nn.Conv1d at the second layer;
conv2_kernel_size = kernel size for torch.nn.Conv1d at the second layer;
conv3_channels = out_channels for torch.nn.Conv1d at the third layer;
conv3_kernel_size = kernel size for torch.nn.Conv1d at the third layer;
n_linear_layers = number of fully connected layers folowing convolutional stack;
linear_channels = out_channels for each fully connected layer folowing convolutional stack;
linear_activation = activation function intervening fully connected layers;
linear_dropout_p = dropout probability between fully connected linear layers;
n_branched_layers = number of branched linear layers after fully connected stack and before output;
branched_channels = number of output channels for each branch of the branched linear layers;
branched_activation = activation function intervening branched linear layers;
branched_dropout_p = dropout probability between branched linear layers;
loss_criterion = loss function to use during training (see torch.nn.loss and custom loss functions in boda2);
parent_weights = path to pytorch state dict to initialze weights for transfer learning;
frozen_epochs = number of epochs at the start of training where transfer learned weights are frozen;
model_module = boda model module used for training;
graph_module = boda graph module used for training;
lr = learning rate;
weight_decay = weight decay regularization;
amsgrad = optimizer setting;
T_0 = scheduler argument;
beta = loss funtion setting;
betas = optimizer settings;
timestamp = YYYYMMDD_HHMMSS timestamp

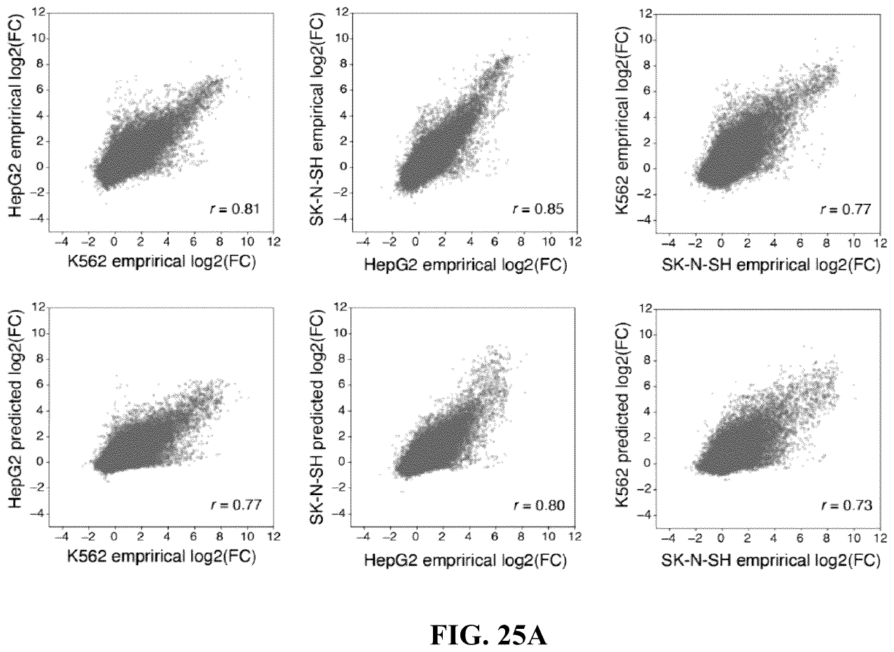

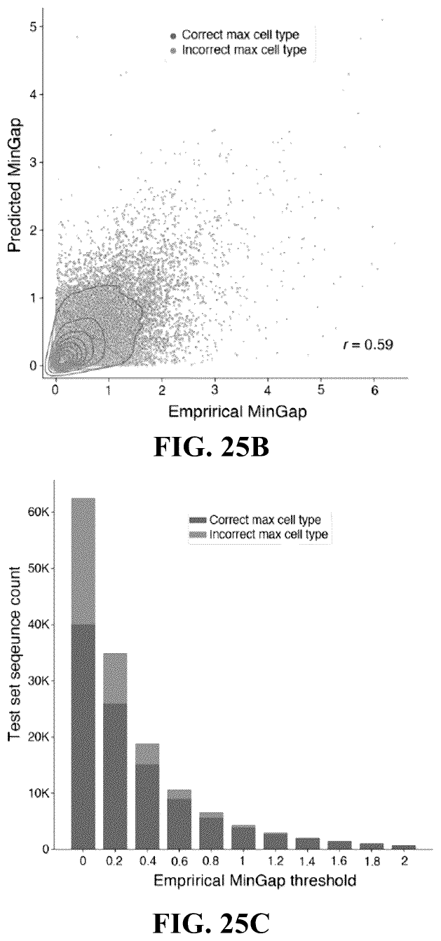

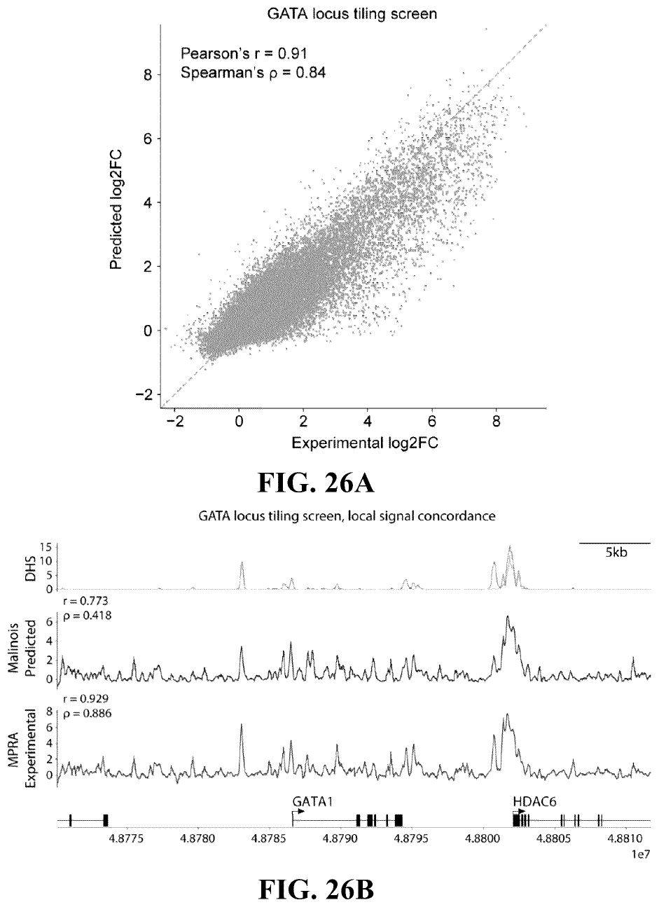

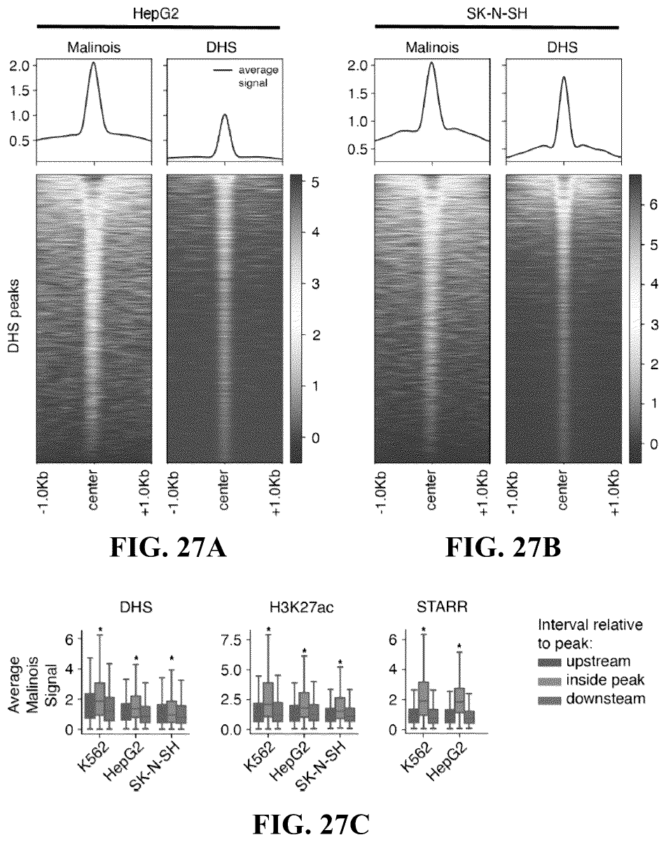

Given Malinois can accurately and rapidly model CRE activity, we generated genome-wide predictions of sequence activity to compare with orthogonal approaches for characterizing CREs. FIG. 25A-25C demonstrates cell type accuracy of model. Applicant observed a strong correlation (Pearson's r=0.91) between Malinois predictions and a comprehensive MPRA of sequences tiling a 2.1 Mb window encompassing GATA1 (FIG. 18E and FIG. 26A-26B). Applicant also found Malinois K562 predictions to have strong activity at known markers of CREs identified by DHS sites59 (p<10⁻³⁰⁰, two-sided paired t-test) and H3K27ac ChIP-seq peaks^{60,61} (p<10-114, two-sided paired t-test), and are correlated with STARR-seq peaks^{60,62} (p<10-178, two-sided paired t-test), an orthogonal measure of CRE activity (FIG. 18F, FIG. 27A-27C, Supplementary Table 1 of Gosai et al. “Machine-guided design of synthetic cell type-specific cis-regulatory elements” BioRxiv doi: doi.org/10.1101/2023.08.08.552077 (2023), which is incorporated by reference as if expressed in its entirety herein)^{5, 63-65}. This finding is consistent in HepG2 and SK-S-SH cells as well (FIG. 27A-27C). Together, this suggests Malinois predictions provide accurate measurements of CREs, approaching the biological reproducibility of empirical measures.

CODA Designs CREs with Desired Functions

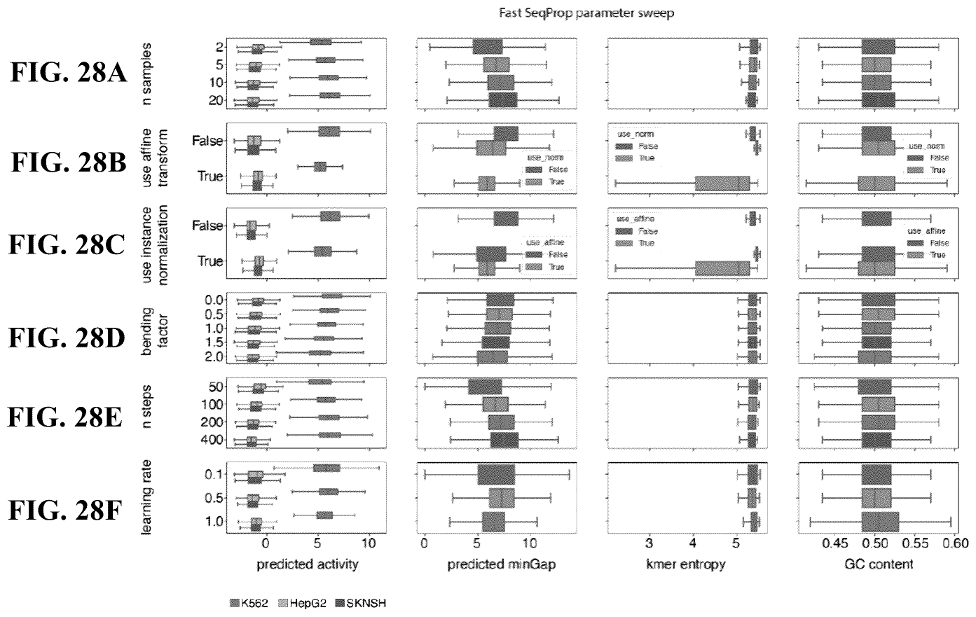

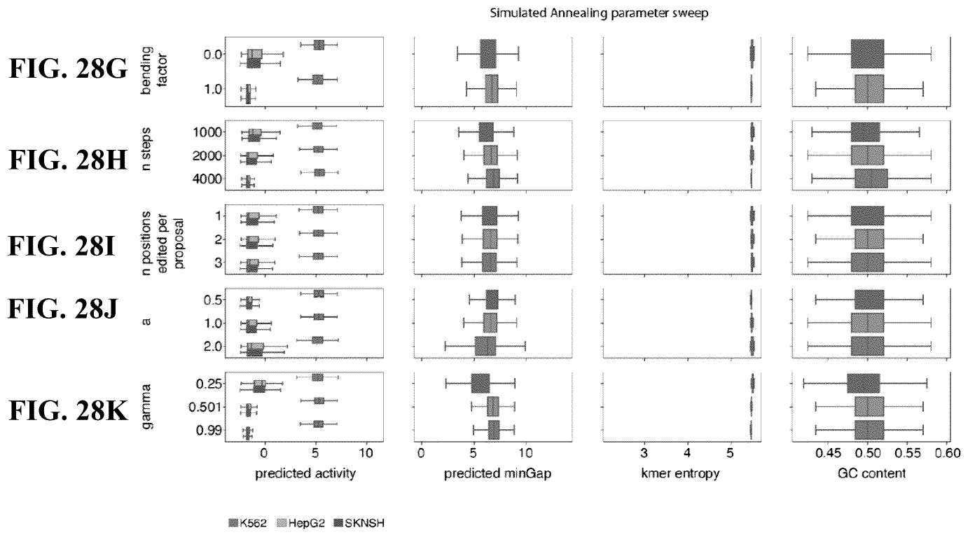

Applicant next developed CODA (Computational Optimization of DNA Activity), a modular platform for designing novel CREs with programmed functionality. CODA follows an iterative loop of predicting the activity of sequences, quantifying how well sequences fit the design goals using an objective function, and then updating sequences to increase the objective value. Here, the goal was to design CREs that drive cell-specific transcription in one of the modeled cell lines, as measured by MPRA. Sequence updates in CODA can be controlled using different classes of sequence design algorithms. We implemented three algorithms representative of three broad classes of optimization techniques (evolutionary: AdaLead³⁵, probabilistic: Simulated Annealing⁶⁶, and gradient-based: Fast SeqProp³⁶) for sequence generation. Applicant selected these methodologies based on their ease of implementation, prior documented successes, or their ability to exploit the structure of deep-learning models. Here, CODA uses Malinois as a fast and accurate measure of CRE activity, efficiently testing millions of CRE designs within the optimization loop. Applicant found the overall ability of these algorithms to design cell-specific elements is generally robust to hyperparameter choices. However, adjustments can be made to balance the tradeoff between maximizing the objective and maintaining k-mer diversity in the set of designed elements (FIG. 28A-28K).

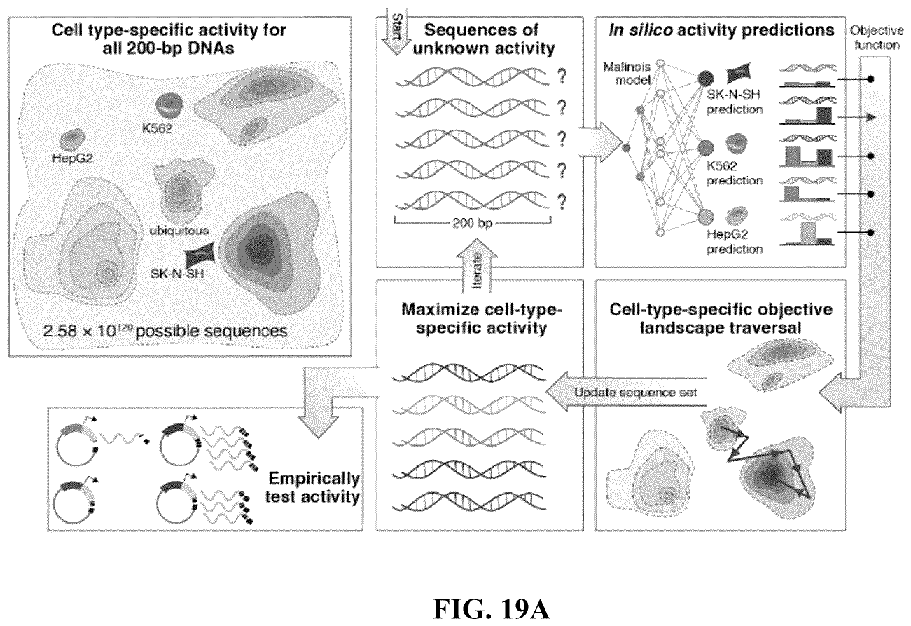

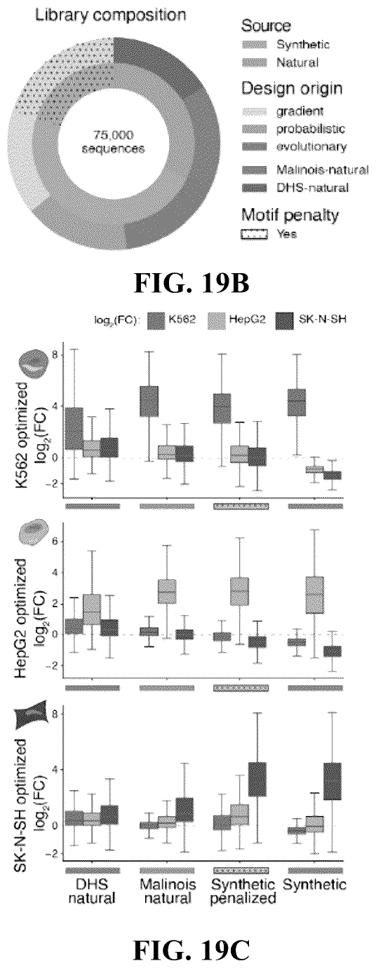

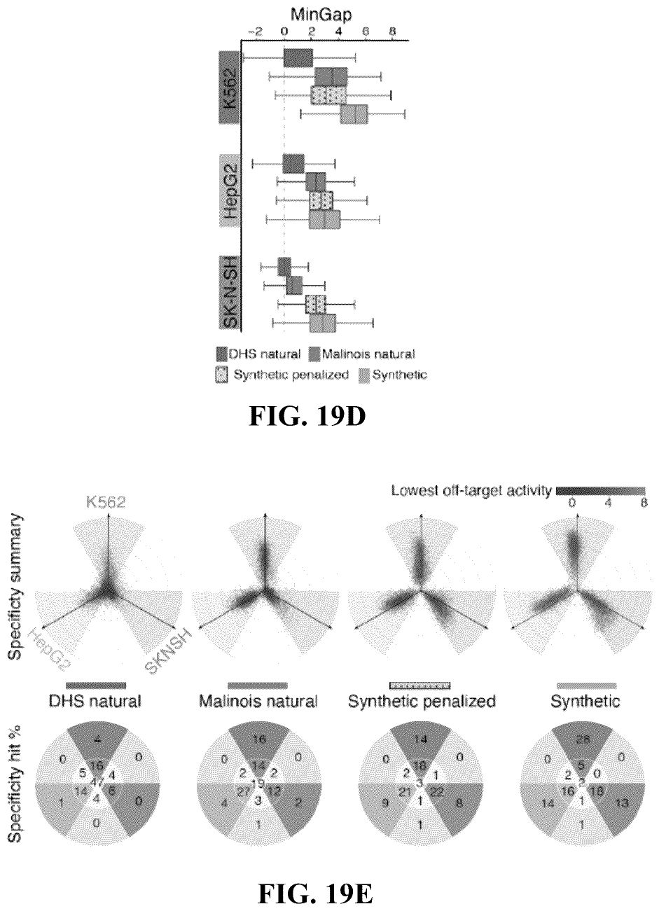

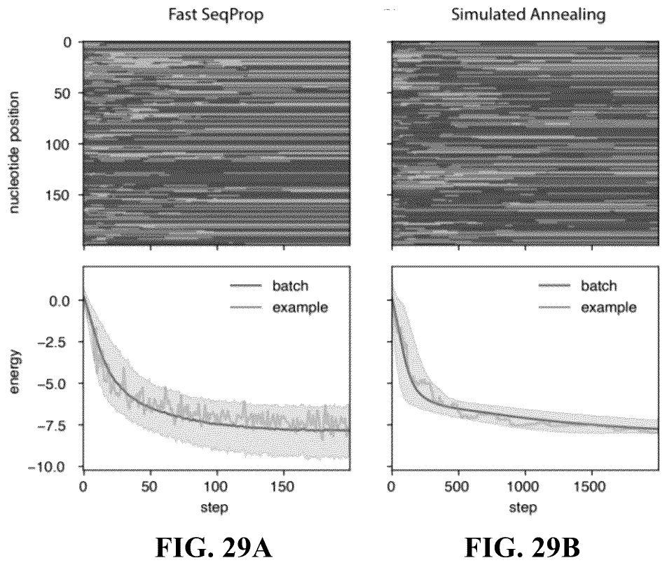

Applicant deployed CODA to rationally design CREs with cell type-specific activity in K562, HepG2, and SK-N-SH cell lines (FIG. 19A). This process involves six steps. Applicant: (i) generated a set of random 200-mer sequences; (ii) predicted regulatory activity of each sequence, in each cell type, using Malinois; (iii) transformed these predictions using an objective function into a single value of cell specificity; (iv) traversed the objective landscape towards specificity by (v) modified the sequence set in silico using one of the design algorithms (FIG. 29A-29B); and (vi) continued iterating until additional updates stop substantially improving the objective value. Applicant defined the objective as a function of the gap observed between predicted MPRA activity in the targeted cell type and the maximum of the two off-target cell types, herein referred to as MinGap (Methods).

To empirically test the effectiveness of CODA, Applicant performed an MPRA to measure activity of the synthetic sequences. For each cell type, Applicant generated 4,000 cell type-specific sequences from each of the three sequence design algorithms in CODA, yielding a total of 36,000 synthetic candidates (FIG. 19B, Table 9, Methods). Applicant observed that Malinois induced strong preferences for certain sequence motifs when maximizing specificity (Supplementary Table 4 of Gosai et al. “Machine-guided design of synthetic cell type-specific cis-regulatory elements” BioRxiv doi: doi.org/10.1101/2023.08.08.552077 (2023), which is incorporated by reference as if expressed in its entirety herein, Table 10, and FIG. 30A). For this reason, Applicant decided to also explore alternative solutions by encouraging CODA to modify the utilization of highly preferred motifs despite the potential decrease in predicted cell type specificity (Methods). Using Fast SeqProp, Applicant designed a second group of synthetic sequences with a motif penalty incorporated into the objective function (FIG. 19B). Over five iterative rounds, Applicant generated a total of 15,000 ‘synthetic-penalized’ CREs, with 1,000 sequences per round per cell type, while penalizing the top motifs from the preceding rounds in each iteration (Supplementary Table 4 of Gosai et al. “Machine-guided design of synthetic cell type-specific cis-regulatory elements” BioRxiv doi: doi.org/10.1101/2023.08.08.552077 (2023)). Applicant observed successful reduction in initially enriched motifs and a simultaneous increase in motifs underutilized in earlier rounds (FIG. 30B), diversifying the syntax of CODA-proposed sequences for experimental evaluation.

TABLE 9

| Axes to parse on: | | | | | | | | |
|---|---|---|---|---|---|---|---|---|
| | | | | | | notes: | floor | |
| Model Type | Basset Branched | | | | | | | |
| Cell Type | K562 | SKNSH | HepG2 | | | Balanced | | |
| Training data | boda/ukbb/gtex | | | | | | | |
| Penalization | none | motif penalization | | | | 24k sequences | 1000 | FastSeqProp/ |
| Strategy | | | | | | | | SimulatedAnnealing |
| Activity score | | | | | | | | |
| bin | | | | | | | | |
| Generator | FastSeqProp | AdaLead | SimulatedAnnealing | | | | | |
| Controls | Negative | Postive | | | | GTEx provides best gold standard | | |
| | | | | | | controls | | |
| | Generators | Cell types | Bins | Penalization | Oligos | In analysis | In experiment | Expected n oligos |
| Primary | 3 | 3 | 1 | 1 | 4000 | TRUE | TRUE | 36000 |
| Penalization | 1 | 3 | 1 | 5 | 1000 | TRUE | TRUE | 15000 |
| Genome-Wide | 1 | 3 | 1 | 1 | 4000 | TRUE | TRUE | 12000 |
| scan | | | | | | | | |
| Best DHS | 1 | 3 | 1 | 1 | 4000 | TRUE | TRUE | 12000 |
| Controls | | | | | | | | 2157 |
| | | | | | | | Total | 77157 |

SUPPLEMENTARY TABLE 10
EME version 4
ALPHABET = ACGT
strands: +−
Background letter frequencies:
A 0.25 C 0.25 G 0.25 T 0.25
MOTIF pos_core_0b
letter-probability matrix: alength = 4 w = 9 nsites = 100
0.17816435 0.334663 0.23974006 0.24743254
0.12733586 0.49374366 0.24161348 0.13730706
0.05902787 0.07433206 0.054291822 0.8123482
0.01262795 0.0053066136 0.004533662 0.9775318
0.99610364 0.0010892533 0.0017191285 0.0010878969
0.0023878522 0.0024950744 0.0022988073 0.99281824
0.0013124568 0.9958475 0.0013886447 0.0014513689
0.27266115 0.09245703 0.22661424 0.4082676
0.19545767 0.26547316 0.3311691 0.20790008
MOTIF pos_core_1
letter-probability matrix: alength = 4 w = 10 nsites = 100
0.1610841 0.41524202 0.21329607 0.21037775
0.17370263 0.38100892 0.23142432 0.21386409
0.45473188 0.14494121 0.25844014 0.14188677
0.06587284 0.6596886 0.098208934 0.17622966
0.10482954 0.038671236 0.053720895 0.8027783
0.0074143144 0.00570726 0.007440896 0.97943753
0.0060008573 0.9838933 0.0051898975 0.0049159084
0.0012205633 0.9961892 0.0013843601 0.0012059436
0.00690395 0.012154835 0.9625836 0.018357603
0.07546303 0.15394221 0.66266894 0.10792586
MOTIF pos_core_2
letter-probability matrix: alength = 4 w = 11 nsites = 100
0.14966147 0.17347696 0.5186751 0.15818645
0.54311657 0.18695611 0.19707936 0.07284798
0.0016418096 0.0030548812 0.0017302632 0.993573
0.0043194336 0.0037881334 0.97901046 0.012881988
0.99059826 0.0038175196 0.0024203113 0.0031638255
0.082036175 0.31956956 0.52785677 0.07053753
0.0016611599 0.0014126666 0.001897866 0.9950283
0.0048230463 0.9917048 0.0016486993 0.0018234885
0.99603736 0.0010520908 0.0018816809 0.0010288189
0.06714473 0.27690476 0.1498188 0.50613177
0.20048784 0.3951135 0.22071043 0.1836882
MOTIF pos_core_3
letter-probability matrix: alength = 4 w = 17 nsites = 100
0.17501636 0.20333841 0.19338939 0.4282558
0.2651902 0.1402877 0.47101128 0.12351086
0.017376112 0.011286033 0.9600697 0.011268217
0.0072430396 0.012085855 0.008060891 0.9726102
0.0112905055 0.013537751 0.009145576 0.9660262
0.99602616 0.00089306873 0.0018444812 0.0012364151
0.9632284 0.016222075 0.009381054 0.01116844
0.028081868 0.017840918 0.010868295 0.94320893
0.16330816 0.45149553 0.21895857 0.1662378
0.94348687 0.011253556 0.019111523 0.026148072
0.015185637 0.012944347 0.020294745 0.9515753
0.0031914972 0.0061197495 0.0027347726 0.987954
0.91879976 0.020631004 0.03241348 0.028155774
0.9300247 0.021554727 0.02940216 0.019018307
0.024149783 0.920474 0.021075686 0.034300555
0.1300228 0.501761 0.15413399 0.21408217
0.4225101 0.19982354 0.20188388 0.17578256
MOTIF pos_core_4
letter-probability matrix: alength = 4 w = 13 nsites = 100
0.3653938 0.1618999 0.33244428 0.14026211
0.030962996 0.025847485 0.91206574 0.031123834
0.18703333 0.14909574 0.22539397 0.438477
0.14448814 0.3339411 0.1757876 0.34578317
0.007319549 0.96713567 0.010996006 0.014548738
0.9752804 0.0053852983 0.012565375 0.0067688865
0.95895815 0.010703972 0.017773824 0.012564065
0.968759 0.009044201 0.013883344 0.008313379
0.0011348622 0.0013291704 0.9961851 0.0013508488
0.029452953 0.020640362 0.058197953 0.89170873
0.08505876 0.5960831 0.09482258 0.2240356
0.006965336 0.9693731 0.010435582 0.01322593
0.69772094 0.066781245 0.1595241 0.07597368
MOTIF pos_core_5
letter-probability matrix: alength = 4 w = 9 nsites = 100
0.1975799 0.23953249 0.17661424 0.38627335
0.08106435 0.10471431 0.18763816 0.62658316
0.7233933 0.046996184 0.17061926 0.058991197
0.001893978 0.9940246 0.0017327095 0.0023488405
0.0017356465 0.001221809 0.9960819 0.00096057495
0.0055106673 0.008008429 0.004581938 0.9818989
0.035039295 0.9312334 0.017306985 0.016420377
0.9220351 0.019953338 0.03831036 0.019701142
0.09335949 0.2894024 0.17924115 0.43799695
MOTIF pos_core_6
letter-probability matrix: alength = 4 w = 12 nsites = 100
0.1503471 0.44651905 0.21840018 0.18473366
0.085507445 0.3396035 0.5077336 0.06715551
0.001069496 0.0014585484 0.9961659 0.0013061874
0.003322414 0.0028124019 0.9895483 0.004316826
0.9623164 0.0104734255 0.016100995 0.011109215
0.9378971 0.023176964 0.016324855 0.022601174
0.650956 0.076112114 0.09833148 0.17460048
0.039053086 0.042346135 0.04331407 0.87528676
0.10600056 0.19957411 0.104580395 0.589845
0.028574595 0.925185 0.02263631 0.023604205
0.017391954 0.9448353 0.021017218 0.016755529
0.13610515 0.5299886 0.20308337 0.13082287
MOTIF pos_core_7
letter-probability matrix: alength = 4 w = 11 nsites = 100
0.21784274 0.15710764 0.48072258 0.14432704
0.22965826 0.13224453 0.36850566 0.26959154
0.07446076 0.019091211 0.8889061 0.017541926
0.0015509648 0.0017390195 0.9951757 0.0015343251
0.0012569824 0.0012048861 0.9961926 0.0013454461
0.118818514 0.71857125 0.05188143 0.11072889
0.0047774445 0.004404932 0.9858007 0.00501694
0.029570302 0.042872537 0.7622395 0.16531768
0.21082008 0.13534759 0.5278807 0.1259516
0.1144279 0.10477987 0.6730265 0.10776567
0.11084818 0.6156607 0.10291517 0.17057592
MOTIF pos_core_10b
letter-probability matrix: alength = 4 w = 9 nsites = 100
0.55166715 0.13936757 0.12564611 0.18331915
0.060188204 0.038695768 0.8810829 0.02003311
0.01678224 0.012998299 0.9573616 0.012857962
0.8107663 0.091922045 0.026426714 0.070885025
0.99618006 0.0014412092 0.0011611512 0.0012176102
0.0010978112 0.002721898 0.0014501434 0.9947301
0.025362272 0.06800303 0.79163545 0.11499925
0.08020247 0.59161586 0.059096087 0.26908556
0.15943572 0.23911873 0.44381258 0.15763296
MOTIF pos_core_12
letter-probability matrix: alength = 4 w = 18 nsites = 100
0.38874015 0.14419936 0.28631604 0.18074451
0.0466431 0.82989913 0.051024213 0.072433524
0.47873336 0.14739934 0.1682708 0.20559652
0.14878803 0.11707767 0.10803543 0.6260989
0.006673383 0.006384567 0.9809534 0.0059887003
0.10951434 0.4764957 0.061437428 0.3525525
0.09805068 0.70006436 0.07957786 0.12230713
0.10376617 0.5297761 0.16894919 0.19750856
0.13381566 0.1024062 0.6929604 0.07081766
0.060170352 0.040510237 0.8498613 0.049458075
0.22861785 0.033510827 0.6674823 0.07038895
0.0011892723 0.99617445 0.0011630416 0.0014731274
0.8317261 0.044687875 0.054046143 0.069539905
0.07942353 0.071828134 0.05939574 0.7893526
0.008363268 0.0056874724 0.98080325 0.0051460247
0.12410478 0.4556528 0.07287836 0.34736404
0.09673545 0.6914375 0.08551416 0.12631291
0.123308636 0.5309995 0.15021718 0.19547471
MOTIF pos_core_14
letter-probability matrix: alength = 4 w = 14 nsites = 100
0.09909686 0.6652199 0.11660817 0.119075075
0.018622985 0.015599828 0.95243007 0.013347154
0.88070405 0.031151524 0.06031665 0.02782785
0.9742285 0.0063699875 0.008088473 0.011312985
0.9724813 0.00932038 0.0075370595 0.010661322
0.15563966 0.41922694 0.3344221 0.090711236
0.03271836 0.8696506 0.028143607 0.06948742
0.0018553905 0.0010711062 0.9960485 0.0010249083
0.9088211 0.027520413 0.041198492 0.022459915
0.9776357 0.0076974365 0.006316203 0.008350653
0.9696623 0.0106461225 0.009139668 0.010551881
0.06250976 0.58490705 0.29873276 0.05385045
0.1124483 0.26541558 0.12727833 0.49485782
0.3361936 0.1346162 0.39538226 0.13380794
MOTIF pos_core_15
letter-probability matrix: alength = 4 w = 9 nsites = 100
0.004395649 0.0049052117 0.003948499 0.98675066
0.0068291454 0.0024122344 0.003146879 0.9876117
0.0017004297 0.9957814 0.0012117224 0.0013063141
0.0370126 0.7267218 0.07734962 0.15891603
0.2414788 0.24108876 0.269268 0.24816442
0.3011007 0.11199723 0.53044254 0.056459498
0.0011616687 0.001100523 0.9961442 0.001593661
0.9890532 0.0029721465 0.0022525562 0.0057221507
0.9874708 0.003661307 0.0048492067 0.0040186574
MOTIF pos_core_16
letter-probability matrix: alength = 4 w = 16 nsites = 100
0.17405045 0.12708826 0.11016002 0.58870125
0.28171986 0.13970117 0.45579153 0.12278743
0.27149642 0.13092215 0.4274667 0.17011477
0.10895455 0.08981868 0.6429116 0.1583152
0.010552374 0.06443112 0.008262444 0.91675407
0.98372525 0.008302046 0.0044063944 0.003566257
0.9949344 0.0024657547 0.001187729 0.0014121515
0.97012335 0.007394201 0.0083588315 0.014123706
0.004743873 0.0401233 0.008457256 0.9466756
0.9955317 0.00082842336 0.0027457655 0.0008940469
0.008221525 0.006748938 0.007568204 0.9774613
0.0014572719 0.0018234948 0.001775919 0.9949433
0.22935095 0.06152223 0.33396825 0.37515855
0.93956614 0.010870725 0.038626183 0.010936985
0.016250553 0.94480616 0.016363963 0.02257932
0.1539142 0.31969473 0.15139575 0.3749953
MOTIF pos_core_21
letter-probability matrix: alength = 4 w = 14 nsites = 100
0.4482465 0.20987359 0.19085008 0.15102981
0.19648725 0.19792683 0.4485148 0.15707113
0.37756616 0.16022076 0.31256068 0.14965245
0.0522985 0.052617528 0.8427693 0.05231465
0.17410126 0.20415692 0.28381127 0.3379305
0.100409895 0.19919217 0.12108208 0.57931584
0.019250007 0.9410296 0.021411102 0.018309245
0.98985845 0.0020966704 0.0049107363 0.0031341582
0.97513944 0.008457946 0.010041032 0.006361583
0.007185264 0.0061259368 0.98217195 0.004516901
0.0012275928 0.0009600109 0.99608386 0.0017284969
0.023271887 0.024663234 0.018116271 0.93394864
0.0037345996 0.9831298 0.0052040555 0.007931514
0.8231561 0.04907273 0.088783346 0.038987797
MOTIF pos_core_22
letter-probability matrix: alength = 4 w = 12 nsites = 100
0.15002903 0.19716169 0.49858132 0.15422794
0.20278077 0.16595334 0.5521984 0.079067506
0.0037438986 0.0047116936 0.0036343008 0.98791015
0.0038650688 0.0045303367 0.012616263 0.9789883
0.8810043 0.00955444 0.09260082 0.016840475
0.031682365 0.68745035 0.035274364 0.24559292
0.012413612 0.0055320105 0.9772563 0.0047981096
0.009393497 0.037624653 0.004240187 0.9487417
0.98666763 0.008130946 0.0031455024 0.0020558753
0.99617577 0.0012875787 0.0014302114 0.001106483
0.08451716 0.5395513 0.17237918 0.20355241
0.08595402 0.6951153 0.101750165 0.11718039
MOTIF pos_core_23b
letter-probability matrix: alength = 4 w = 9 nsites = 100
0.06217687 0.7161003 0.10874109 0.112981774
0.06369643 0.7293516 0.10316513 0.103786856
0.18864253 0.0969781 0.12514648 0.58923286
0.023234379 0.027586607 0.025802271 0.92337674
0.0011055195 0.0016803086 0.0010966973 0.9961175
0.01025656 0.005731306 0.980336 0.0036761125
0.018282808 0.011393676 0.006325125 0.9639984
0.11544264 0.112009905 0.3671631 0.40538433
0.10108936 0.30500284 0.087063946 0.50684386
MOTIF pos_core_26
letter-probability matrix: alength = 4 w = 10 nsites = 100
0.37875023 0.2524608 0.26159373 0.10719528
0.03723438 0.04684496 0.034572665 0.881348
0.0054432233 0.9849555 0.004833947 0.0047673346
0.4715066 0.09280047 0.33165026 0.104042634
0.00095861184 0.99609214 0.0012522571 0.0016969665
0.0017992284 0.001288816 0.99598503 0.0009268906
0.11238127 0.1635169 0.068935655 0.6551662
0.0055022817 0.0060078264 0.9815391 0.006950721
0.9390138 0.017135818 0.025385741 0.01846458
0.10160371 0.33362088 0.17550157 0.38927385
MOTIF pos_core_27b
letter-probability matrix: alength = 4 w = 7 nsites = 100
0.008930705 0.0047842385 0.9809724 0.00531258
0.0022499475 0.013384568 0.0015181557 0.98284733
0.99566156 0.0025172788 0.001055825 0.0007654614
0.99518627 0.0026654592 0.0010498507 0.0010984492
0.95408636 0.010802367 0.018859323 0.016251866
0.0029363553 0.96535814 0.004903136 0.02680235
0.9737269 0.007125256 0.011173654 0.007974188
MOTIF pos_core_30
letter-probability matrix: alength = 4 w = 12 nsites = 100
0.46826458 0.17179239 0.20462447 0.15531851
0.018578393 0.017634591 0.9480214 0.015765699
0.7338242 0.064923085 0.09734839 0.10390438
0.03867621 0.02894882 0.032426137 0.8999489
0.0008038029 0.9958871 0.0012972085 0.0020117701
0.9960582 0.0009854559 0.0018218327 0.001134539
0.9916415 0.0022283725 0.0035143315 0.0026157186
0.97552425 0.0076013613 0.009350869 0.0075234715
0.0052790577 0.0060352213 0.98456347 0.004122235
0.17063299 0.1471736 0.51972485 0.16246857
0.16342089 0.24870533 0.31831276 0.269561
0.10701995 0.6242544 0.11921174 0.14951392
MOTIF pos_core_31
letter-probability matrix: alength = 4 w = 10 nsites = 100
0.73727494 0.0743956 0.11366854 0.07466101
0.017507013 0.91422033 0.032366194 0.035906505
0.028756753 0.015060974 0.020949233 0.935233
0.006716262 0.005022585 0.006545207 0.981716
0.003962563 0.9890837 0.0035102833 0.0034435373
0.0011928742 0.9961882 0.0013898573 0.0012290528
0.055914365 0.11780155 0.3076706 0.5186135
0.10829734 0.28764668 0.46321312 0.14084291
0.17431608 0.23373519 0.17371382 0.41823488
0.17287739 0.20024747 0.15783796 0.46903723
MOTIF pos_core_32b
letter-probability matrix: alength = 4 w = 10 nsites = 100
0.25461814 0.14753139 0.12020085 0.47764957
0.29669812 0.09774903 0.5277308 0.07782202
0.6840216 0.1009836 0.11173443 0.10326044
0.63195086 0.06241314 0.19628863 0.109347396
0.001884017 0.9878028 0.0023513408 0.007961861
0.996097 0.001678534 0.0012650492 0.0009593523
0.99147487 0.003575745 0.002607856 0.0023416027
0.98078716 0.004706923 0.0072322083 0.0072736754
0.020262832 0.87317264 0.041593 0.064971544
0.93334186 0.021686893 0.028599247 0.016371889
MOTIF pos_core_33
letter-probability matrix: alength = 4 w = 11 nsites = 100
0.12457308 0.06912253 0.72863823 0.07766613
0.1602027 0.6550117 0.0934468 0.09133881
0.09306046 0.0648685 0.68106395 0.1610071
0.07260999 0.77601665 0.072266 0.079107314
0.121893376 0.048705176 0.76283485 0.06656664
0.013257212 0.9382223 0.017518582 0.031001918
0.001566153 0.0010669695 0.99614763 0.0012192584
0.002012467 0.99358726 0.0016512532 0.0027490144
0.0054045254 0.004037403 0.986075 0.0044830544
0.0998678 0.69080955 0.07416753 0.13515513
0.10993971 0.11684404 0.66373485 0.10948139
MOTIF pos_core_34
letter-probability matrix: alength = 4 w = 18 nsites = 100
0.48937804 0.16320428 0.17542914 0.17198853
0.48581803 0.15470074 0.1935097 0.16597153
0.2587028 0.42004105 0.21819401 0.1030621
0.026386015 0.9398073 0.021627035 0.012179721
0.0034338566 0.005082067 0.98766893 0.0038150616
0.0029983788 0.0026277215 0.9917481 0.0026257976
0.9950765 0.0016230394 0.0017129662 0.0015875568
0.99264824 0.0014952276 0.0018764061 0.003980137
0.90247023 0.031401616 0.04182188 0.024306282
0.16642609 0.41164646 0.22505072 0.1968767
0.056830067 0.7983315 0.0614692 0.083369285
0.0017935598 0.0012058215 0.9960588 0.00094181724
0.92093194 0.026708288 0.029727733 0.022632059
0.96232164 0.013092604 0.010321448 0.01426417
0.95055836 0.017064072 0.015408924 0.016968682
0.0614243 0.6701676 0.20984408 0.05856397
0.12029012 0.25774026 0.13734102 0.48462856
0.32395482 0.14335857 0.39803195 0.1346547
MOTIF pos_core_39
letter-probability matrix: alength = 4 w = 12 nsites = 100
0.16103019 0.21175674 0.20009118 0.42712194
0.0048968415 0.005703658 0.98514855 0.004250976
0.053841222 0.045921452 0.78918004 0.11105725
0.9258569 0.023480574 0.025736108 0.024926404
0.8731243 0.043522626 0.039333586 0.044019554
0.5753467 0.0775065 0.07992967 0.26721713
0.06153038 0.0428962 0.036159974 0.8594134
0.014065132 0.0115712015 0.012711817 0.9616518
0.006246099 0.005859581 0.005118038 0.9827763
0.0065031787 0.9864184 0.0035417038 0.003536641
0.0010970038 0.99615884 0.0015306879 0.001213395
0.48974752 0.14572906 0.25313175 0.111391656
MOTIF pos_core_44
letter-probability matrix: alength = 4 w = 12 nsites = 100
0.108613275 0.094612405 0.6285591 0.16821522
0.19726983 0.54137444 0.13866888 0.12268687
0.03424452 0.9118052 0.0342554 0.019694757
0.005404559 0.003981784 0.98219126 0.008422385
0.015296945 0.96463335 0.008967864 0.011101839
0.0013464176 0.99619246 0.0012597598 0.0012013601
0.9863732 0.004254047 0.0057872524 0.0035854261
0.001684374 0.0018133993 0.0015470134 0.99495524
0.15488566 0.5002993 0.15300536 0.19180976
0.045149878 0.027888238 0.032623768 0.89433813
0.019845394 0.033679657 0.020739894 0.925735
0.1692198 0.15923232 0.50300574 0.16854209
MOTIF pos_core_46
letter-probability matrix: alength = 4 w = 14 nsites = 100
0.17749749 0.15507284 0.49949172 0.16793798
0.30166686 0.22626114 0.3113278 0.16074422
0.09500752 0.6674628 0.12794755 0.109582074
0.11220833 0.32703352 0.17529996 0.3854582
0.10932248 0.27593458 0.5866719 0.028071053
0.003017608 0.99245036 0.0025770029 0.001955024
0.0027776018 0.0012113863 0.9936953 0.0023156728
0.0011200099 0.9961747 0.0012509208 0.0014543389
0.32130134 0.6186595 0.033437237 0.026601892
0.028982555 0.09892306 0.036733378 0.83536094
0.06174186 0.04189989 0.8634882 0.032870114
0.014891138 0.94606096 0.012335702 0.026712231
0.05203027 0.09555454 0.76254934 0.08986586
0.06840011 0.6905692 0.09828658 0.14274411
MOTIF pos_core_51b
letter-probability matrix: alength = 4 w = 10 nsites = 100
0.6052561 0.10510636 0.2176261 0.07201149
0.041793697 0.15410958 0.08444101 0.71965575
0.98194855 0.007828429 0.00582871 0.0043942477
0.03164713 0.025314914 0.024465451 0.9185725
0.0013823808 0.002182635 0.9931486 0.0032863854
0.02301807 0.95481455 0.009625119 0.01254234
0.109138645 0.05428503 0.045630954 0.79094535
0.976789 0.0075930697 0.00969695 0.005920949
0.99584794 0.001572585 0.0018970452 0.0006823367
0.038914908 0.18170722 0.31012937 0.46924853
MOTIF pos_core_57b
letter-probability matrix: alength = 4 w = 12 nsites = 100
0.16466296 0.112373725 0.5405273 0.18243603
0.010144853 0.96345586 0.010473545 0.015925739
0.0021512855 0.007120418 0.004376704 0.9863516
0.99387604 0.0015594471 0.0020677394 0.0024968018
0.11938184 0.05072834 0.045691606 0.78419816
0.25426662 0.043474626 0.05757848 0.64468026
0.5299475 0.0977388 0.058436204 0.3138775
0.94037104 0.012516135 0.015020688 0.03209202
0.0014273445 0.0014014862 0.0010185223 0.9961526
0.9806497 0.0053778077 0.011089957 0.0028825356
0.02155501 0.013489874 0.9520031 0.012952057
0.14022776 0.6695926 0.095476605 0.09470309
MOTIF neg_core_0
letter-probability matrix: alength = 4 w = 10 nsites = 100
0.22131267 0.4346475 0.12998493 0.21405491
0.30852643 0.28538677 0.11843044 0.2876564
0.19202177 0.24589434 0.30749145 0.25459236
0.36636448 0.102234796 0.13085277 0.400548
0.004070597 0.0025918346 0.9901494 0.0031880748
0.99415994 0.0019568868 0.0020502182 0.0018329474
0.0014595657 0.0013260519 0.001052732 0.9961617
0.010407034 0.006587373 0.009019843 0.97398573
0.1382535 0.18597871 0.19513977 0.48062804
0.3375276 0.2178901 0.20401049 0.2405718
MOTIF neg_core_5
letter-probability matrix: alength = 4 w = 11 nsites = 100
0.20647885 0.21032862 0.22029686 0.3628956
0.64494646 0.09864594 0.12040697 0.13600054
0.13391477 0.6825644 0.07426748 0.10925338
0.97904223 0.0074928263 0.0058584902 0.0076064565
0.011561807 0.012518921 0.96528983 0.010629374
0.006710817 0.007082491 0.9800846 0.006122063
0.001395003 0.0013868061 0.0010532084 0.99616504
0.028014038 0.011403819 0.94467753 0.015904678
0.1570082 0.20513453 0.1196332 0.51822406
0.2879343 0.1611573 0.374847 0.1760614
0.44619107 0.21101202 0.14408958 0.19870733
MOTIF neg_core_6
letter-probability matrix: alength = 4 w = 10 nsites = 100
0.08942345 0.76296115 0.08711561 0.06049988
0.0830795 0.7386743 0.058359995 0.11988617
0.006561341 0.0034126656 0.0072841 0.9827419
0.0046821157 0.002852532 0.989253 0.0032123413
0.0014389225 0.0011261707 0.99617755 0.001257416
0.015184319 0.8665877 0.010525396 0.107702576
0.9937448 0.0017270258 0.0025068454 0.0020213288
0.05528609 0.7695993 0.049760364 0.12535422
0.13229133 0.6472725 0.092757136 0.12767902
0.2131249 0.23983076 0.17462055 0.37242374
MOTIF streme_1
letter-probability matrix: alength = 4 w = 13 nsites = 100
0.65934277 0.05562047 0.14862372 0.13641301
0.301757 0.30395383 0.18330325 0.21098596
0.10880358 0.60481477 0.10585493 0.18052666
0.077333905 0.7763427 0.047317687 0.09900564
0.14466675 0.13900168 0.4739317 0.24239986
0.0024837193 0.00092170946 0.0008980784 0.9956965
0.0022335716 0.9923137 0.0025143 0.0029383276
0.02436304 0.026836155 0.8957319 0.053068917
0.97353154 0.0054967036 0.0091102915 0.011861463
0.60999274 0.0847427 0.18113643 0.124128096
0.12123869 0.1026756 0.66159064 0.114495076
0.4853594 0.1436117 0.18617982 0.18484916
0.28003588 0.11632246 0.18319169 0.42045
MOTIF streme_2
letter-probability matrix: alength = 4 w = 11 nsites = 100
0.55500627 0.11693044 0.13414098 0.19392222
0.5626846 0.07291685 0.14908041 0.21531808
0.40451723 0.20813233 0.16493738 0.22241308
0.011798373 0.0075626746 0.97118187 0.009457054
0.9779549 0.004471908 0.009728917 0.00784419
0.0012527746 0.0014718835 0.0011061857 0.99616915
0.040588174 0.028644836 0.89297897 0.037788074
0.061256796 0.7860406 0.079122335 0.07358029
0.106997766 0.1596274 0.06552356 0.66785127
0.40856084 0.26951185 0.13496117 0.1869661
0.32518893 0.17250574 0.24257809 0.25972724
MOTIF streme_3
letter-probability matrix: alength = 4 w = 10 nsites = 100
0.09456632 0.70929873 0.07636977 0.11976516
0.9779026 0.0052758874 0.0075321435 0.009289323
0.1404783 0.2903089 0.48112592 0.08808693
0.084407054 0.7049119 0.13331926 0.07736188
0.0013604835 0.0022823557 0.0011213734 0.99523586
0.0048341216 0.003137381 0.98796797 0.0040606107
0.0022942682 0.0020194084 0.0016596651 0.99402666
0.007854589 0.96948177 0.008731938 0.013931673
0.8776236 0.03703934 0.03812121 0.04721576
0.94621503 0.012902666 0.01751546 0.023366863
MOTIF streme_4
letter-probability matrix: alength = 4 w = 10 nsites = 100
0.27050126 0.1464786 0.20029129 0.38272884
0.11872582 0.062019594 0.18903537 0.63021916
0.011748171 0.007736609 0.97077 0.009745207
0.0040338514 0.002127119 0.9919259 0.001913116
0.0010734556 0.0018967098 0.0010003297 0.9960295
0.9960295 0.0010003297 0.0018967098 0.0010734551
0.001913116 0.9919259 0.002127119 0.0040338514
0.009745207 0.97077 0.007736609 0.011748166
0.6302192 0.18903539 0.062019594 0.11872583
0.38272884 0.20029129 0.1464786 0.27050126
MOTIF streme_5
letter-probability matrix: alength = 4 w = 10 nsites = 100
0.19747405 0.078936234 0.07432188 0.64926785
0.16309533 0.23241328 0.072963566 0.5315278
0.11716555 0.6677018 0.08649356 0.12863912
0.0031156053 0.0009733278 0.0006700297 0.99524105
0.02643124 0.0399616 0.006936386 0.92667073
0.055430055 0.17185 0.72906303 0.043656897
0.97203875 0.003426894 0.011308105 0.013226194
0.9889563 0.0033514316 0.0027380614 0.0049542524
0.05007354 0.025153922 0.8829504 0.041822195
0.65327996 0.05345966 0.14651528 0.14674515
File follows MEME motif format: meme-suite.org/meme/doc/meme-format.html

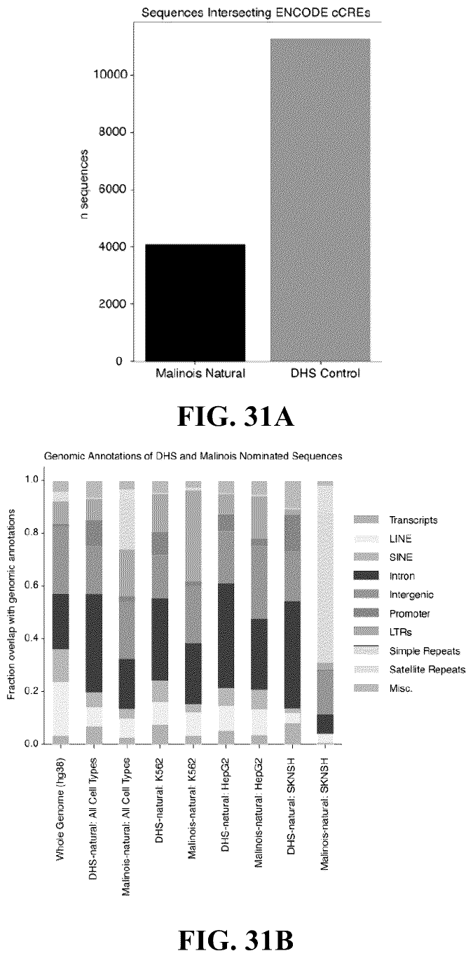

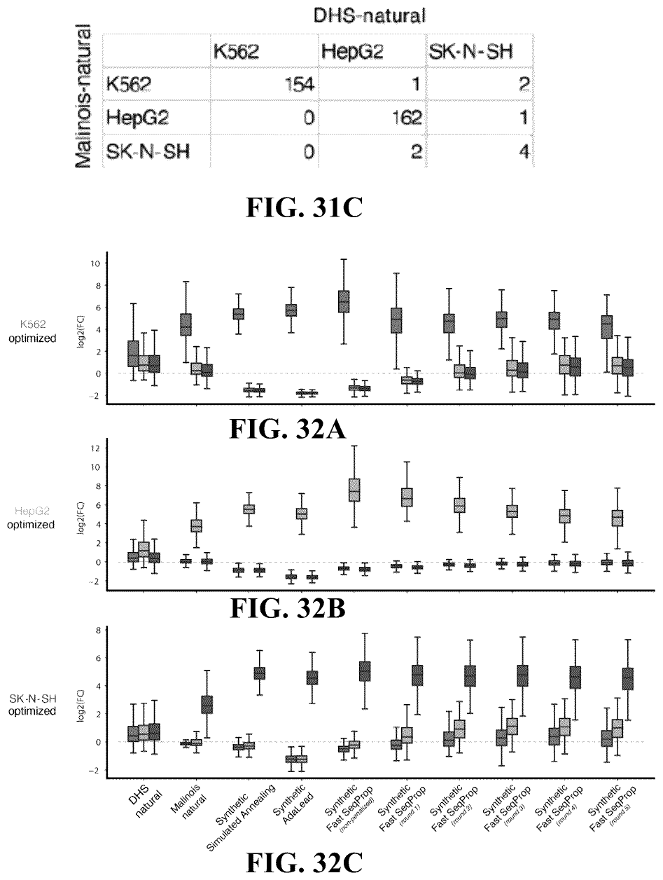

Applicant also selected naturally occurring CREs from the human genome to investigate how well these sequences drive cell type-specific activity compared to our synthetic designs. H3K27ac histone marks and chromatin accessibility as measured by DHS are common proxies for active CREs^{6,59}. Thus, for each cell line we identified 4,000 ‘DHS-natural’ sequences with cell type-specific chromatin accessibility and overlapping H3K27ac signals (12,000 total) (Methods). Applicant then scanned the entire human genome for 200-mers predicted to be cell type-specific by Malinois and selected 4,000 ‘Malinois-natural’ sequences with the greatest on-target expression and minimal off-target expression in each of the three cell lines (Methods, FIG. 31A). Notably, there was low overlap between elements identified using DHS or Malinois (0.10%-4.1% intersection depending on cell type of interest, FIG. 31C). Although DHS-natural sequences displayed high levels of chromatin accessibility, Malinois-natural and both synthetic groups were predicted to have greater cell type specificity, with non-penalized synthetic sequences surpassing all groups (FIG. 32A-32C).

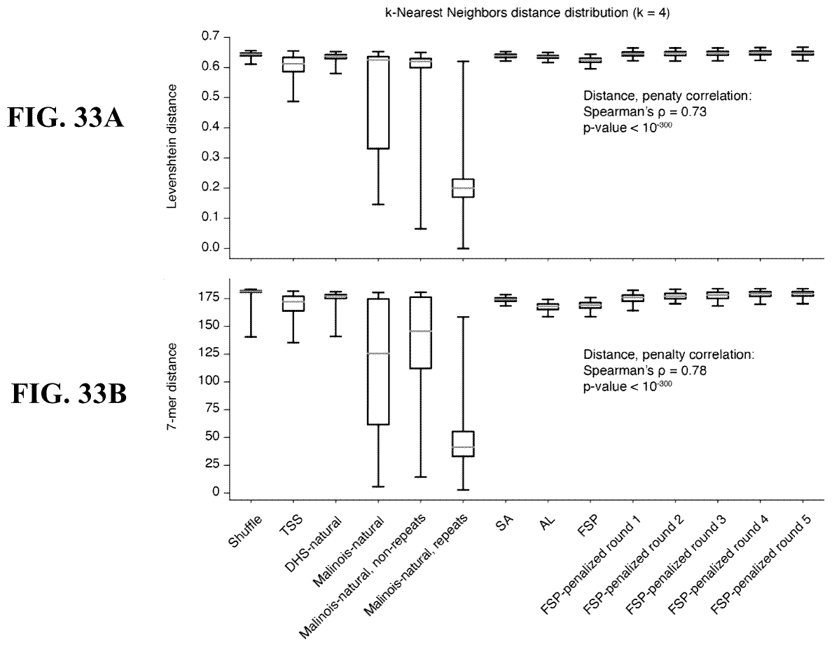

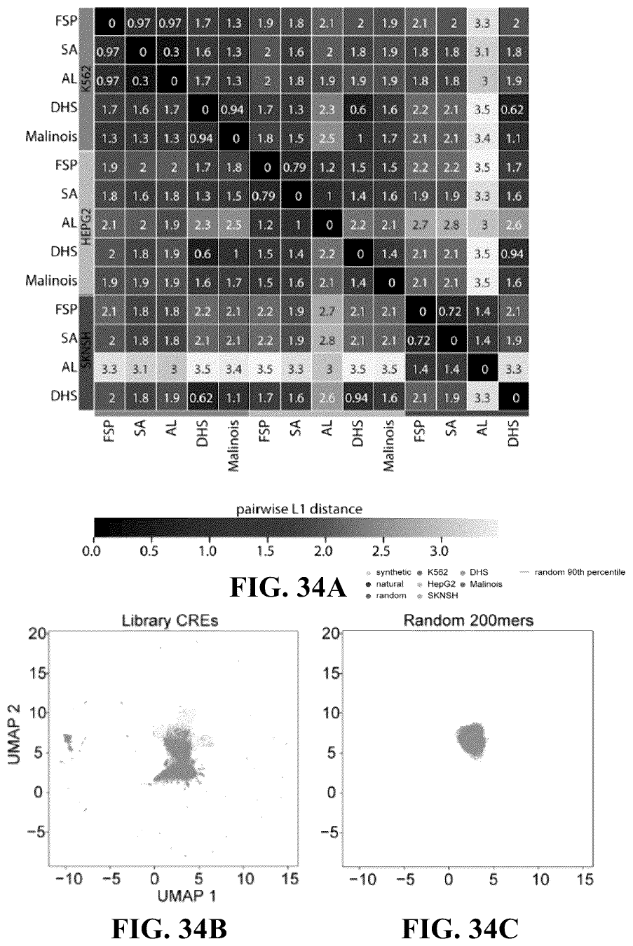

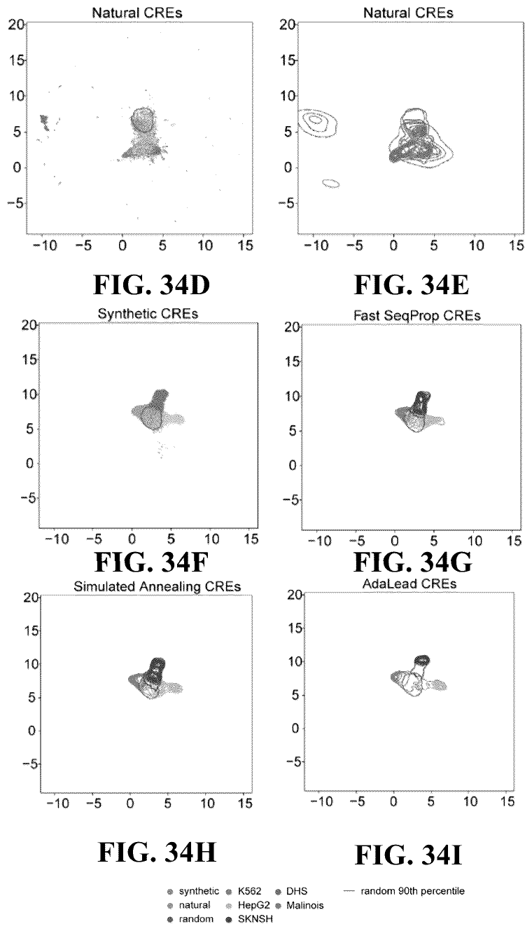

All methods used to generate synthetic CREs resulted in groups of sufficiently diverse sequences. Applicant first quantified single-nucleotide similarity by calculating the average Levenshtein distance of each sequence to its 4 nearest neighbors within the corresponding design group, and repeated this process for human promoters and shuffled sequences from the library as controls (FIG. 33A). DHS-natural, and non-repetitive Malinois-natural sequences were respectively 1.2%, and 11.8% closer to neighbors than shuffled controls. Depending on the generative algorithm, non-penalized synthetic sequences were 0.57%-2.9% closer to neighbors. Interestingly, synthetic-penalized sequences were on average 0.45%-0.89% further away from their 4 nearest neighbors than shuffled controls, with distances increasing during successive penalization rounds (Spearman's ρ=0.73 p<10-300). In contrast, promoters were 8.9% closer to neighbors than shuffled controls, implying that synthetic sequences are substantially more diverse than promoters. As a more stringent assessment of diversity that can capture reuse of individual sequence motifs, we also quantified the average distance of 7-mer content to the 4 nearest neighbors for all oligos. On average, non-repetitive natural sequences selected by DHS and Malinois were 3.0% and 24.4% closer to their nearest neighbors, respectively, than shuffled sequences. Synthetic sequence pairs showed median levels of 7-mer diversity in between groups of natural sequences, being on average 3.6%-7.2% closer to nearest neighbors than shuffled sequences. Motif penalization significantly reduced neighbor closeness from 6.5% to 0.82% relative to shuffled controls (Spearman's ρ=0.75, p<10⁻³⁰⁰, FIG. 33B). On the other hand, despite the modest reductions compared to shuffle sequences, all groups except Malinois-natural showed less 7-mer similarity than promoters (on average 9.7% closer to nearest neighbors than shuffled sequences), showing synthetic sequences provide a diverse collection of CREs. Finally, embedding the 4-mer content of the sequences into two-dimensions using UMAP we observed synthetic elements separated by target cell type and from natural elements (FIG. 34A-341) supporting the observation that the synthetic sequences are distinct to sequences found in the human genome⁶⁷.

CODA Successfully Generates Synthetic CREs with High Cell Type Specificity

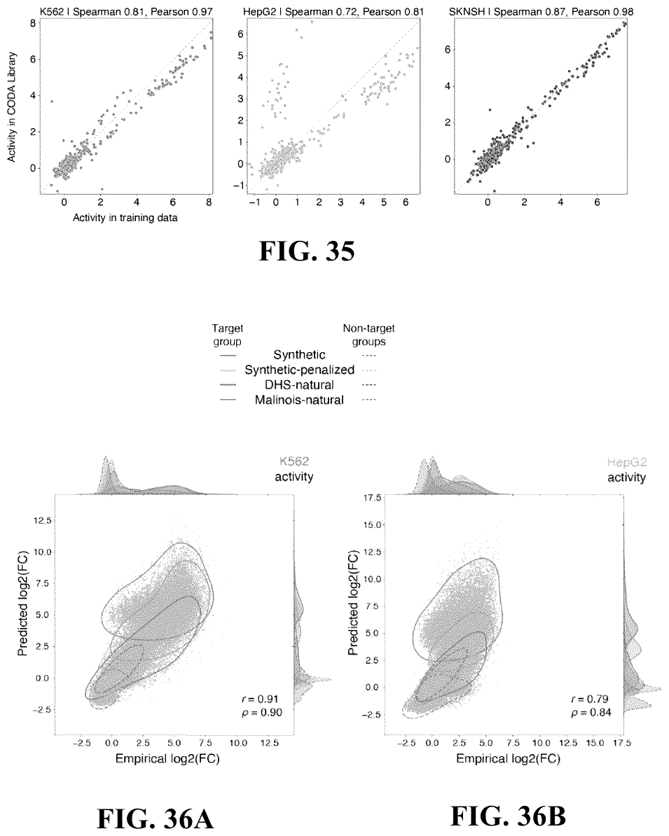

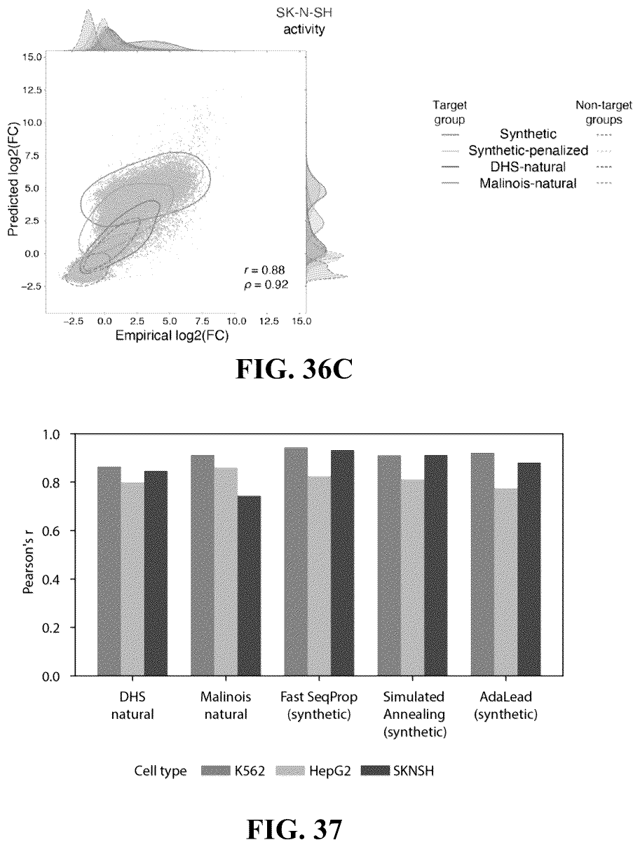

Applicant experimentally tested the library of 77,157 natural and synthetic sequences (FIG. 19B) to determine if machine-guided sequence design could reliably generate biologically functional elements with desired activity. In total, the library included 51,000 synthetic sequences (36,000 standard and 15,000 motif-penalized), 24,000 natural sequences (12,000 DHS-natural and 12,000 Malinois-natural), and 2,157 experimental controls. Applicant quantified activity of an individual CRE as the log₂ fold change (log ₂FC) of expression of the reporter gene driven by the CRE compared to a set of negative controls (FIG. 19B-19C). A set of 594 control elements shared with the training data libraries confirms the high reproducibility of MPRA measurements across experiments (Pearson's r 0.97, 0.81, and 0.98 for K562, HepG2, and SK-N-SH, respectively; FIG. 35). Malinois prospectively predicted empirical MPRA measurements of this library with high accuracy (Pearson's r 0.79-0.91; Spearman's ρ 0.84-0.92; FIGS. 36A-36C and FIG. 37), suggesting Malinois' predictive accuracy is not limited to natural sequences.

Applicant was able to identify naturally occurring sequences with cell type specificity, with Malinois-natural sequences significantly outperforming DHS-natural sequences, suggesting that DHS and H3K27ac peaks are a poor predictor of specificity in MPRA. To quantify cell type-specific expression between design groups we used the MinGap score, which is the log₂FC in the target cell type minus the maximum off-target log₂FC. Consistent with a priori Malinois activity predictions of genomic sequences, DHS-natural sequences in all three cell types performed poorly as cell type-specific CREs compared to natural sequences identified by Malinois (median MinGap difference Malinois-natural vs DHS-natural: K562 2.78, HepG2 1.84, SK-N-SH 0.57; p<10-258 for all, one-sided Wilcoxon rank-sum test) (FIG. 19D, FIGS. 32A-32C, FIGS. 38A-38C, and FIGS. 39A-39C). These differences in MinGap were primarily driven by weaker on-target activity for DHS-natural sequences compared to Malinois-natural in K562 (median log₂FC: DHS-natural 2.06, Malinois-natural 4.54) and HepG2 cells (DHS-natural 1.44, Malinois-natural 2.72), while low on-target activity in SK-N-SH in both groups (DHS-natural 0.64, Malinois-natural 0.84) resulted in a lower MinGap difference and reduced SK-N-SH specificity observed in natural sequences in general.

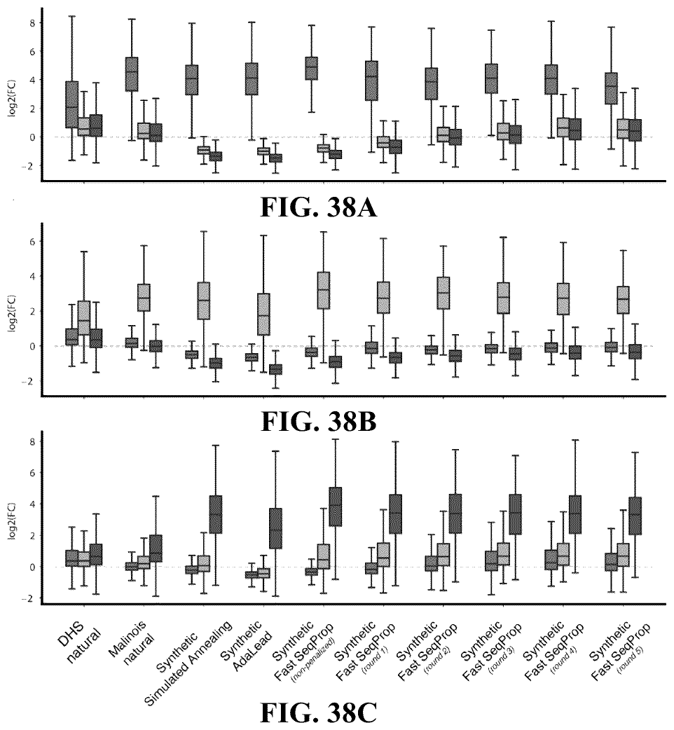

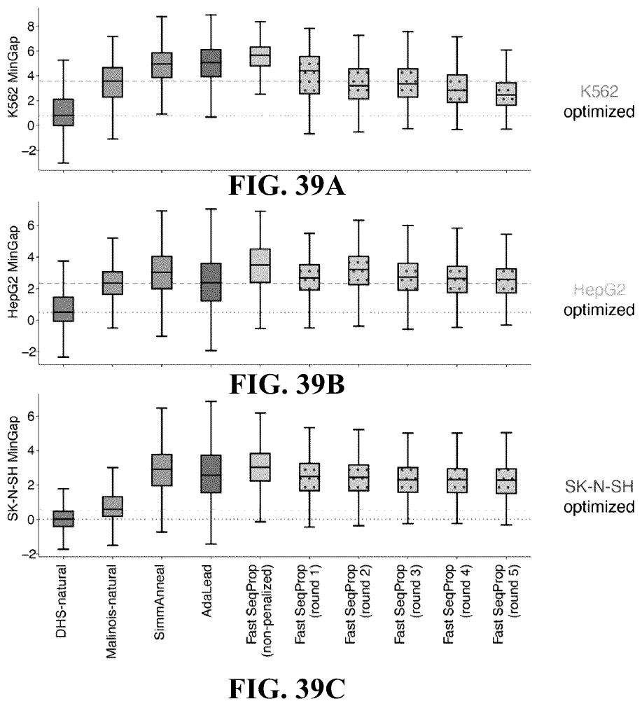

Synthetic sequences from all three algorithms outperformed both groups of natural sequences as cell type-specific CREs in all three cell types. Compared to Malinois-natural, the best performing natural sequence group, all synthetic designs displayed a higher MinGap for all target cell types (median MinGap difference synthetics vs Malinois-natural: K562 1.70, HepG2 0.65, SK-N-SH 2.28; p<10-121 for all, one-sided Wilcoxon rank-sum test) (FIG. 19D, FIGS. 38A-38C, and FIGS. 39A-39C). Between design methodologies, Fast SeqProp demonstrated greater consistency and slightly higher MinGap across all cell types (Mean MinGap difference Fast SeqProp: 0.41 over Simulated Annealing, 0.62 over AdaLead; p-adj<10⁻³⁰⁰, Tukey's HSD test). Performance gains for all synthetic groups were primarily driven by greater repression in off-target cell types (median off-target log₂FC: synthetic −0.69, Malinois-natural 0.09, DHS-natural 0.41). In addition, synthetic sequences had a higher on-target activity in SK-N-SH (median log₂FC 3.20) compared to both natural groups, and higher on-target activity for HepG2 and K562 compared to DHS-natural sequences (FIG. 19C). In summary, synthetic sequences consistently achieved the largest quantitative separation between target and off-target cell types when compared to both classes of naturally derived sequences.

In addition to evaluating specificity using MinGap, Applicant quantified and visualized specificity utilizing all three cell measurements. Applicant developed a radial coordinate system where the most specific sequences trend outwards along one of the three cell type axes, while sequences with uniform activity across cell types are drawn toward the origin (FIG. 19E, Methods). The system incorporates both the MinGap and the MaxGap (log₂FC separation between the target cell type and minimum off-target) scores. Applicant categorized CREs as cell type-specific if two conditions are met: (i) the MaxGap is greater than 1, and (ii) the MinGap: MaxGap ratio is greater than 0.5. These two requirements prioritize sequences with on-target preference while avoiding sequences in which one off-target cell type is closer to the target cell type than the other off-target cell type (Methods).

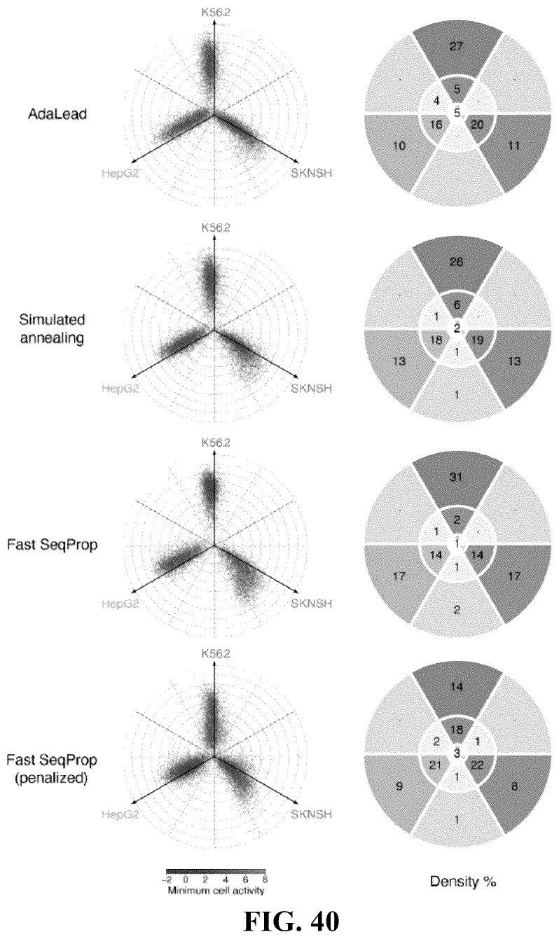

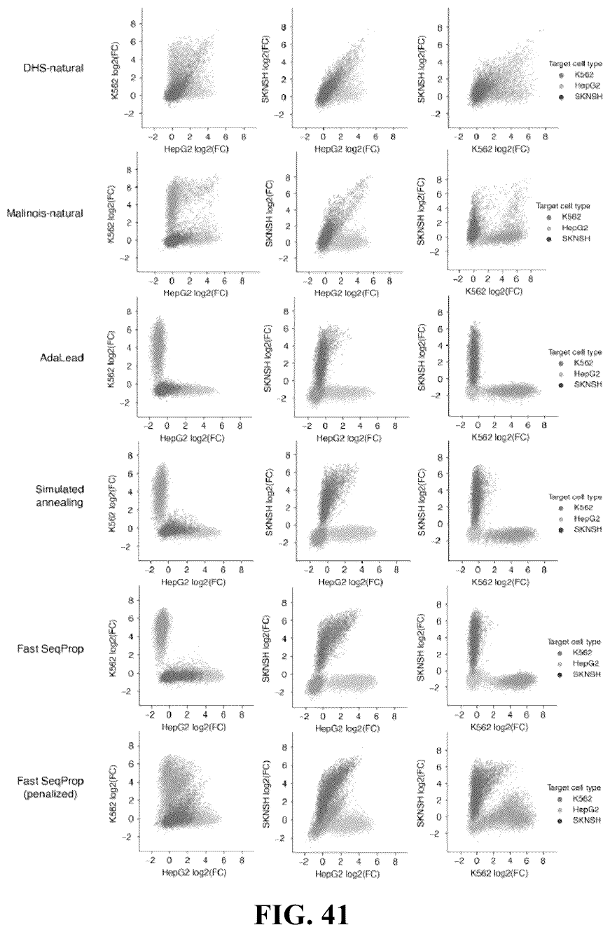

Using Applicant's criteria to categorize cell type-specific CREs, Applicant observed that most (94.1%) synthetic sequences designed by CODA successfully drive cell type specificity (FIG. 19E, FIG. 40, and FIG. 41). Depletion of the most optimal motifs did not impact success substantially, with 92.4% of motif-penalized sequences still driving specificity. Comparatively, we observe that Malinois-natural (73.6%) and DHS-natural sequences (40.6%) were less successful (FIG. 19E). When increasing the stringency of the MaxGap four-fold, synthetic sequences (54.7% specific) further outperformed Malinois-natural (21.5%) and DHS-natural (4.7%) sequences, as well as motif-penalized sequences (30.8%). Overall, synthetic CREs lacking any homology to the human genome (Methods) more consistently drive robust cell-specific activity in large part through repression of off-target activity, as well as through some increases in on-target activity.

### The TF Vocabulary of Synthetic Sequences Drives Cell Type-Specific CRE Activity

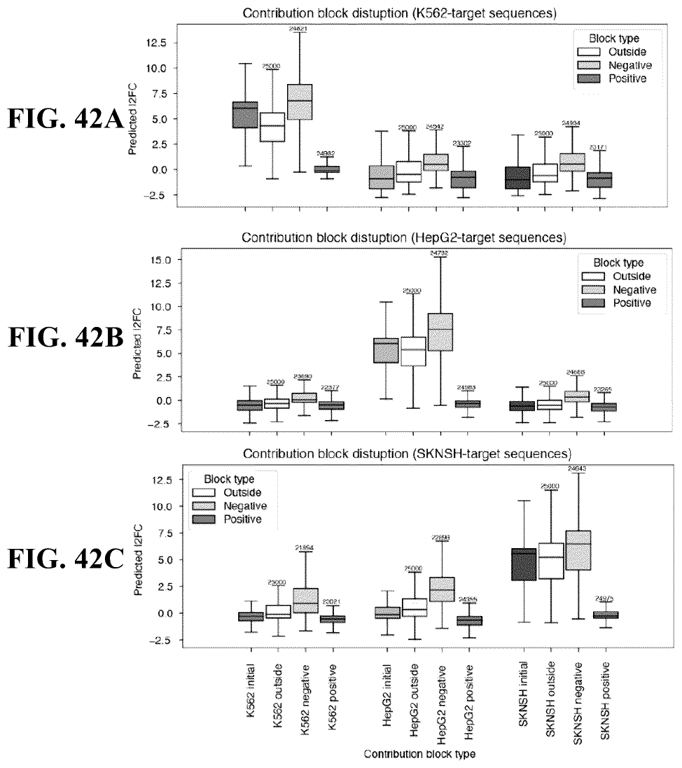

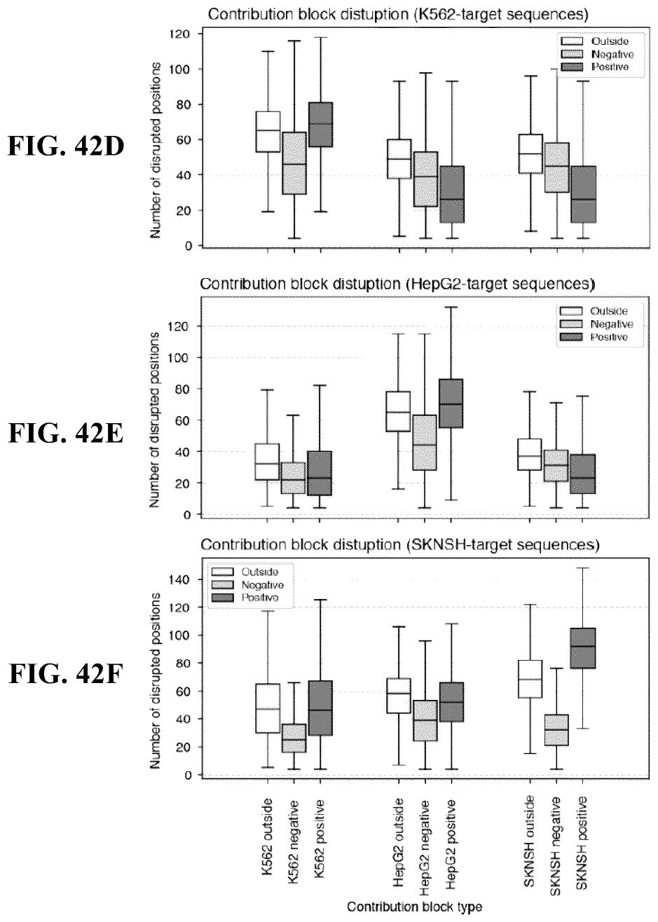

Having found that synthetic CREs are more cell type-specific than both classes of natural sequences, Applicant sought to link sequence content to the responsible regulatory syntax. Transcription is controlled in part by individual TF binding to sequence motifs as well as interactions between TFs10. First, Applicant used Malinois to predict nucleotide-resolution activity contribution scores for each sequence in the three cell types using a modified version of Integrated Gradients (Methods) 68. Applicant consistently observed that disrupting blocks of positive contribution led to a decrease in predicted activity, while disrupting blocks of negative contribution resulted in an increase (FIG. 42A-42F, Methods). This alignment with expected prediction effects supports the functional relevance of the contribution scores as perceived by the model. Next, we employed TF-MoDISco Lite^{69,70} to identify 66 motif patterns informed by contribution scores, from which Applicant extracted 36 non-redundant core motifs (7-18 bp) enriched in our MPRA-tested library, with 31 confidently aligning to a known human TF binding motif (FIG. 43A-43D, Methods, Table 10) ^{71,72}.

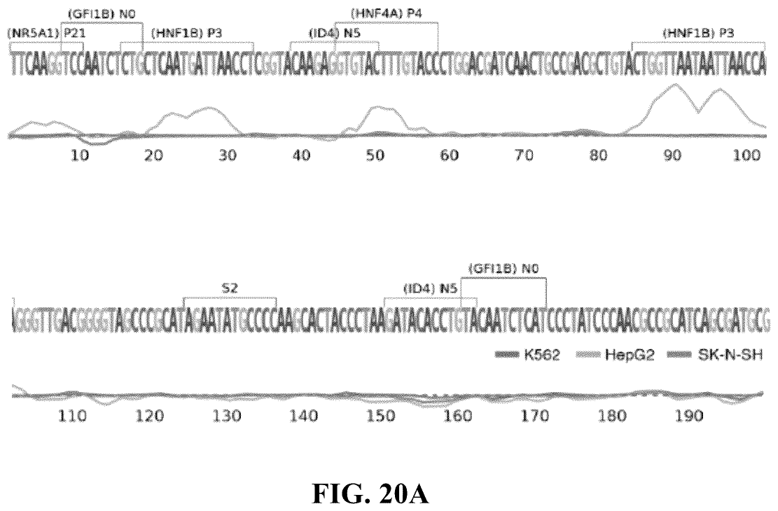

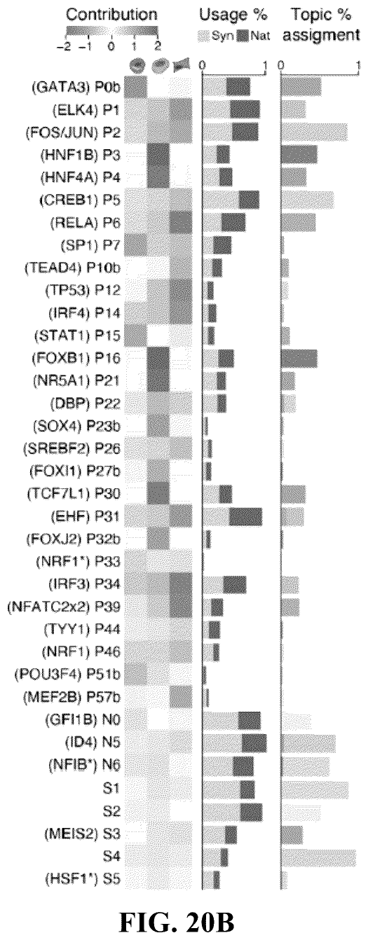

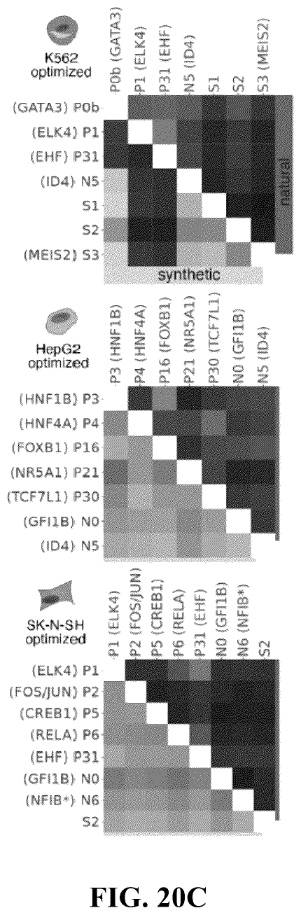

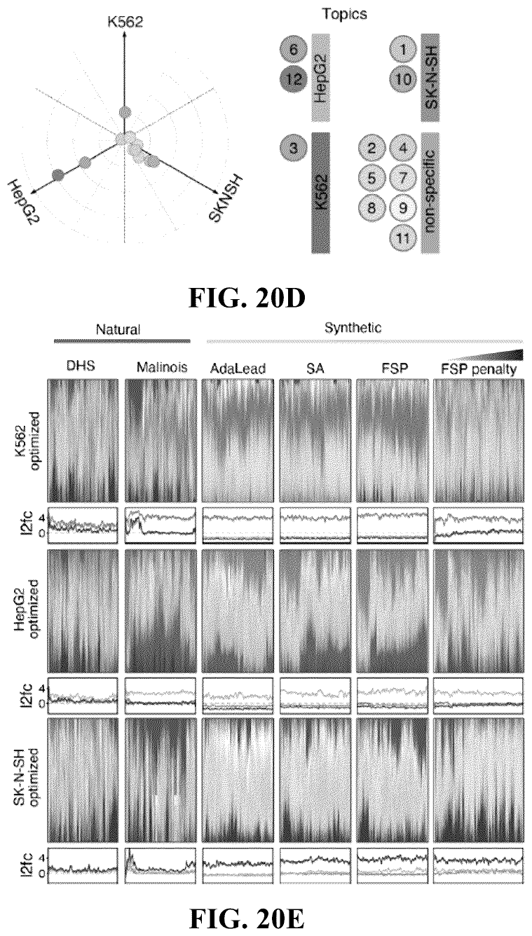

The regulatory activity contribution scores identify the overall magnitude and direction of the effect of each motif in each of our three cell lines (FIG. 20A). Of the 36 core motifs, 28 had positive predicted contributions to sequence activity while the remaining 8 were repressive. This included well-known activators such as GATA73, a heavily utilized and essential TF expressed in K562, which is correctly predicted by Malinois to drive activity exclusively in K562 (FIG. 20B).

Likewise, HNFIB and HNF4A, master regulators expressed in hepatocyte development⁷⁴⁻⁷⁷, are used to drive transcription in HepG2 cells and their contributions are exclusive to HepG2. Motifs displaying negative contributions included the repressors GFI1B in K562⁷⁸⁻⁸⁰, and MEIS2 in HepG2 and SK-N-SH⁸¹⁻⁸³. All motifs demonstrated predicted effects in accordance with their assigned contribution when embedded in a random background, as well as when replacing their instances in the library with random sequences (FIG. 43A-43D, FIG. 44A-44C, Methods).

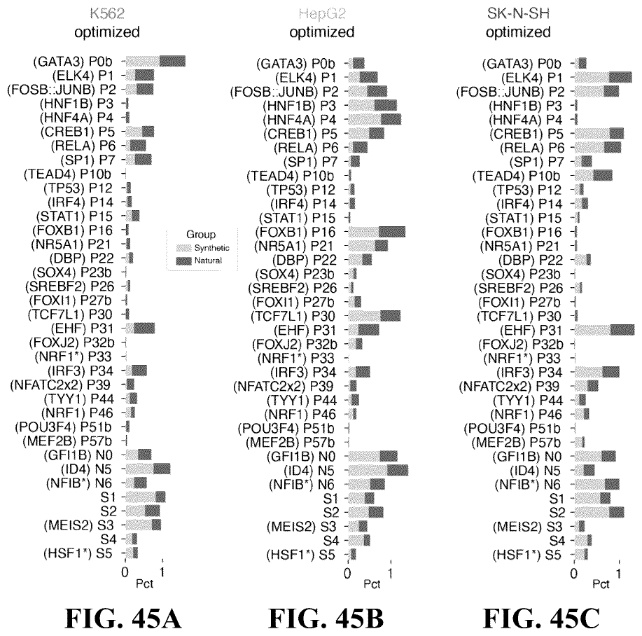

Applicant examined whether motif use differed between natural and synthetic sequences using a contribution score-based motif hit mapping (Methods, Supplementary Table 7 of Gosi et al. “Machine-guided design of synthetic cell type-specific cis-regulatory elements. Nature. In Review. 2024, which is incorporated by reference as if expressed in its entirety herein). All of the 36 core motifs occur at least once in both synthetic and natural sequences, suggesting a shared vocabulary between the two classes (FIG. 20B, FIG. 45A-45C). However, the utilization of motifs differed. For example, motifs for transcriptional activators GATA in K562 and HNF4A in HepG2 were deployed at higher rates in synthetic sequences (all synthetics: 92.3%, 77.1%, respectively; all naturals: 69.8%, 47.2%, respectively), as well as the repressors MEIS2 in K562 and GFI1 B in HepG2 (all synthetics: 71.4%, 74.5%, respectively; all naturals: 24.6%, 40.8%, respectively) (FIG. 45A-45C).

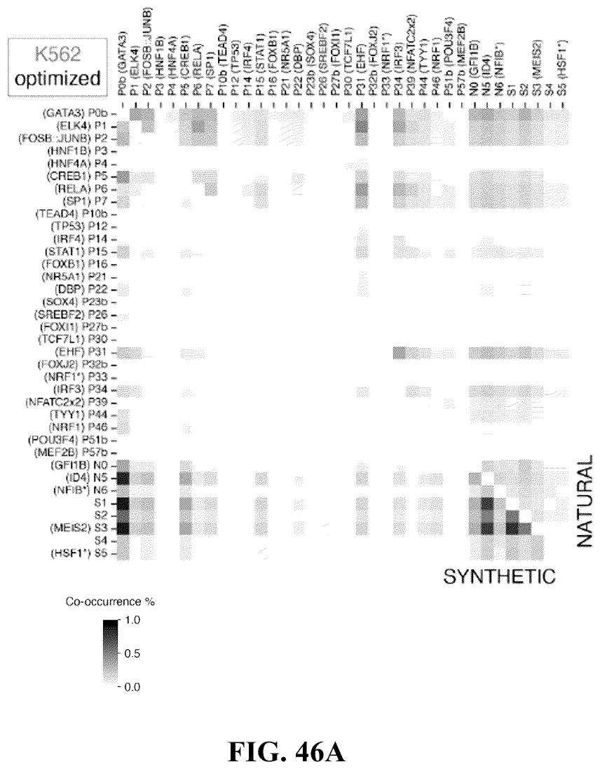

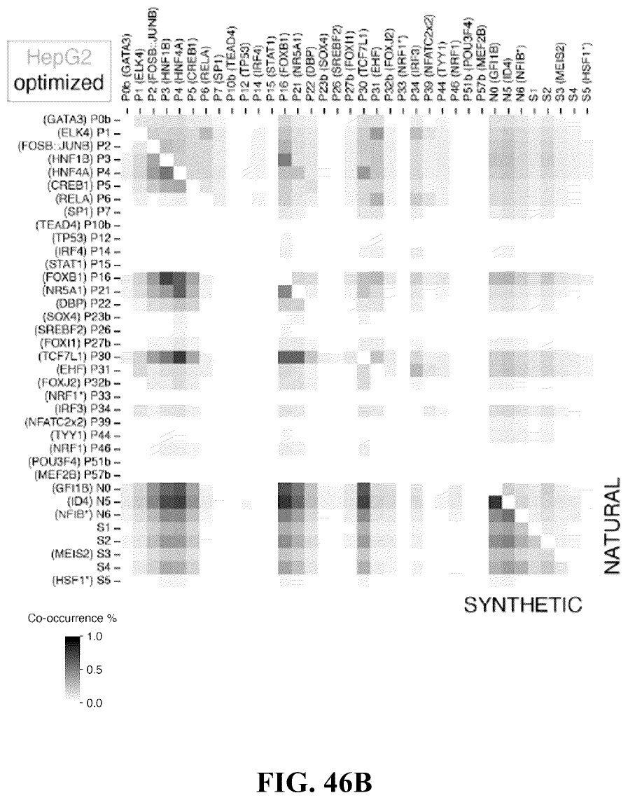

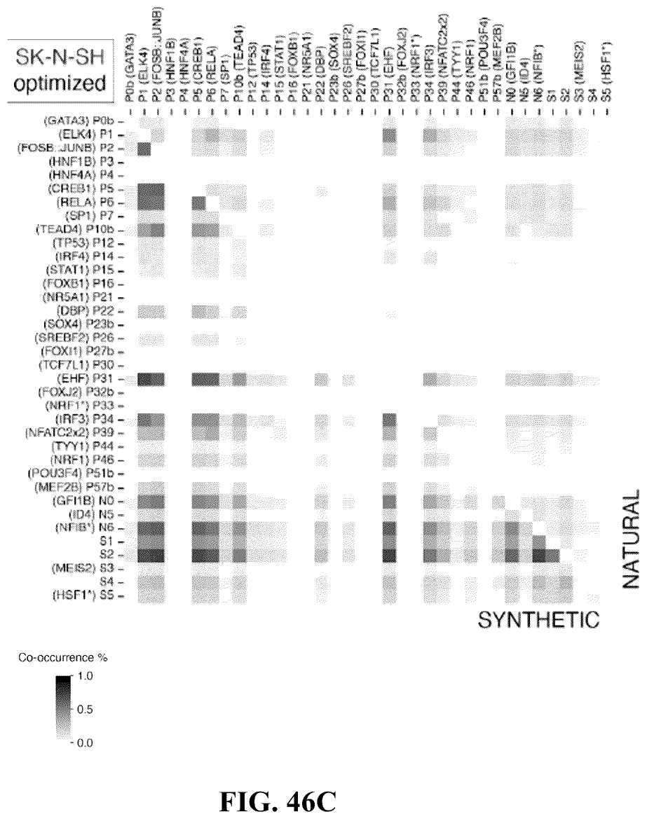

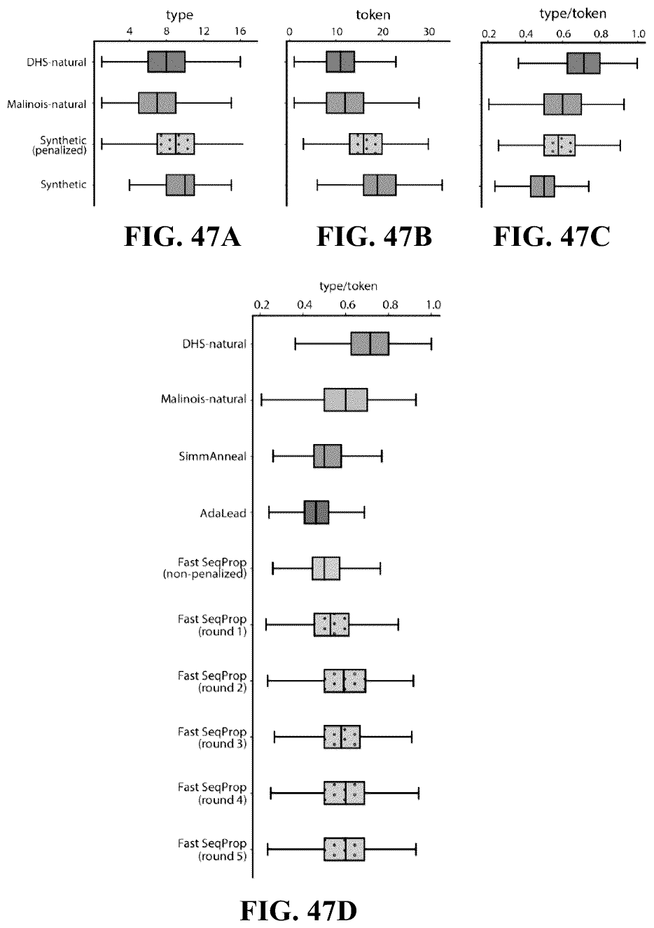

Notably, Applicant also observed a higher use of particular motif combinations in synthetic sequences that were subtly present in natural sequences. For example, among non-penalized synthetic sequences, Applicant see higher rates of GATA/MEIS2 in K562 (89.2%) and HNF4A/GFI1 B in HepG2 (64.6%), compared to natural sequences (17.9%, 18.8% respectively) (FIG. 20C, FIG. 46A-46C, Methods). Combinations of two distinct activating motifs were observed in most non-penalized synthetic and Malinois-natural sequences (95.7% and 93.4%, respectively), while activating-repressive and repressive-repressive motif pairs were observed at lower rates in the natural group (activating-repressive: synthetic 99.9%, Malinois-natural 83.1%; repressive-repressive: synthetic 98.9%, Malinois-natural 57.6%), suggesting that natural sequences are less likely to use repressive grammar in constructing cell type-specific CREs. Further emphasizing the increased use of individual and combinations of motifs in synthetic sequences, we observe that non-penalized synthetic elements showed a greater diversity of unique motifs (types) per sequence (2 more types in median vs natural; p<10-300, one-sided Wilcoxon rank-sum test) as well as a greater number of total motif instances (tokens) (7 more tokens in median vs natural; p<10-300, one-sided Wilcoxon rank-sum test) per sequence (FIG. 47A-47B). As expected, penalization rounds for synthetic sequences reduce some individual motif instances, reducing both types and tokens (1 more type in median vs natural; 4 more tokens in median vs natural). However, the type: token ratio, a measure of non-redundant motif deployment, is higher in penalized synthetic sequences than in non-penalized ones due to reduced motif redundancy (median type: token 0.58 vs 0.5 respectively; p<10-300, one-sided Wilcoxon rank-sum test; FIG. 47C-47D). As these sequences remain highly specific, CODA is able to explore alternative regulatory mechanisms successfully despite increased syntactical constraints posed by penalization.

### Complex Semantic Architectures are Syntactically Differentially Deployed in Natural and Synthetic Sequences

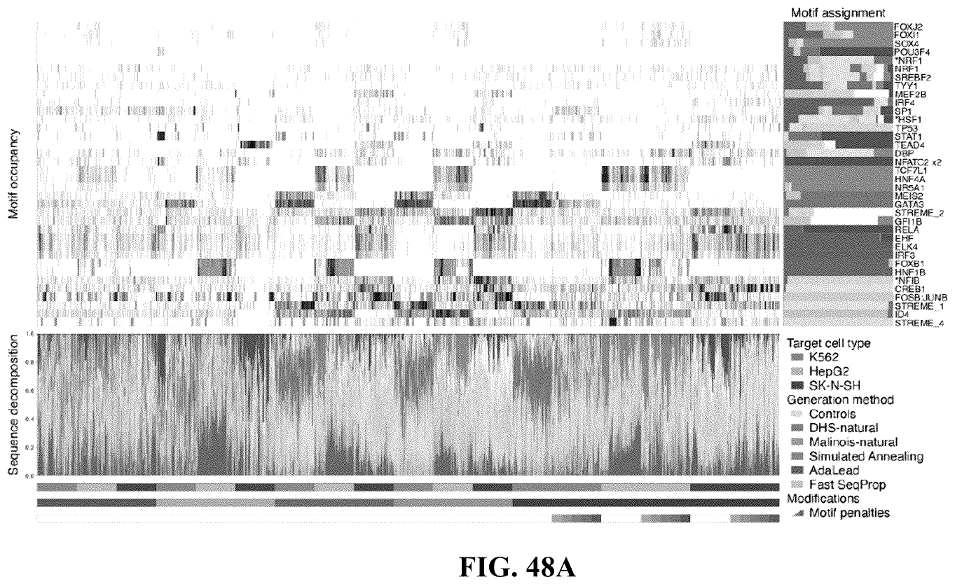

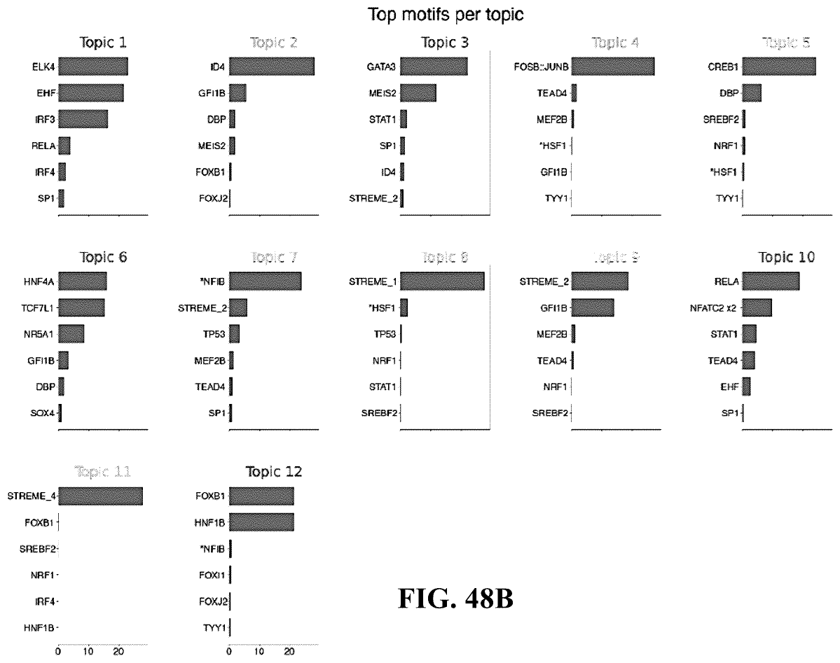

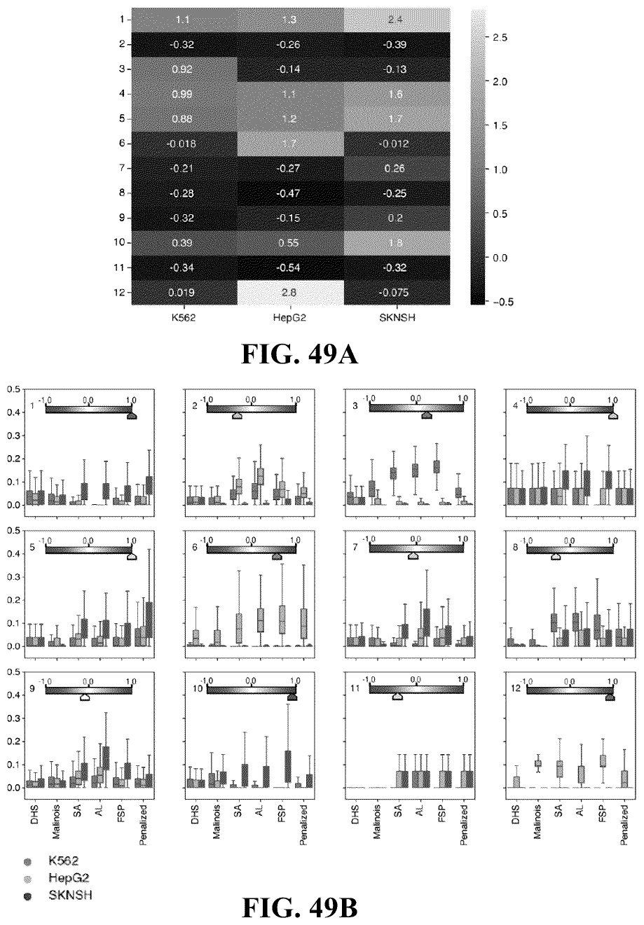

In addition to single TF-motif usage and pair-wise co-occurrence, cell type specificity is thought to arise through higher-order motif semantics, which can mediate the complex organization of many TFs to impart CRE activity^{7, 8, 10, 11}. To aggregate semantically-related motifs into functional programs, Applicant used Non-negative Matrix Factorization (NMF) 84 to decompose sequences in our library into a mixture of 12 functional programs based on motif content calculated using contribution score-based motif mapping (FIG. 48A-48B, Methods). These programs broadly describe related sequences found in the elements Applicant tested. NMF identified 5 programs associated with clear cell type-specific activity (1 program in K562, and 2 in each HepG2 and SK-N-SH), with the 7 remaining programs associated with pleiotropic activation and/or repression (FIG. 20D, FIG. 49A).

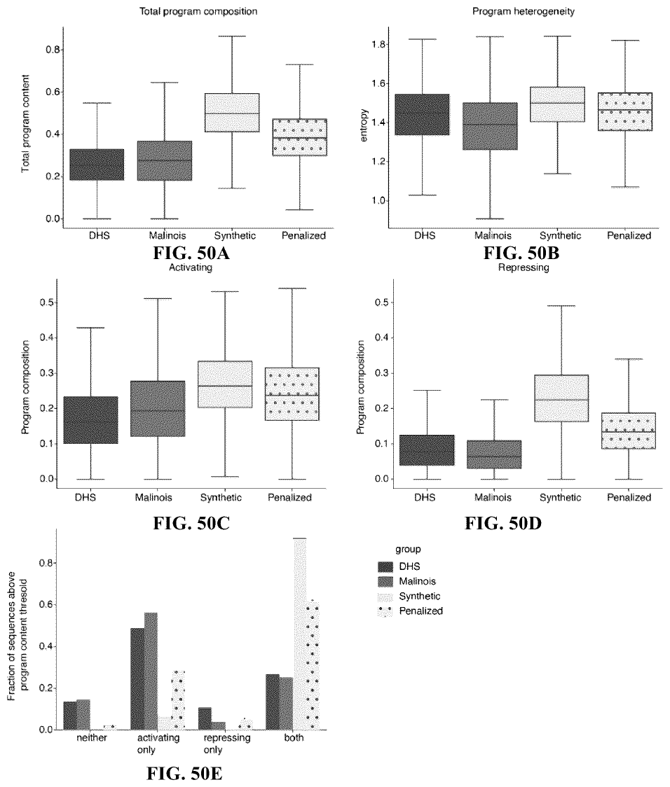

Natural and synthetic sequences deploy distinct distributions of semantic programs (FIG. 20E, FIG. 49B). While there are quantitative differences in program preference between the different synthetic sequence design methods, there are no programs unique to one method. Overall, synthetic elements have higher program content and program heterogeneity compared to natural CREs (FIG. 50A-50B). Applicant also found that natural sequences primarily rely on activating programs while synthetic sequences also frequently utilize programs with repressive effects in off-target cell types (median repressing program content: DHS-natural 0.077; Malinois-natural 0.064; synthetic 0.123) (FIG. 50C-50D). The vast majority of synthetic sequences (91.9%) are composed of both activating and repressing programs each exceeding a threshold of 0, while relatively fewer DHS (26.9%) and Malinois (25.3%) natural sequences show this combination (Methods, FIG. 50E). These results support Applicant's motif-based observations that the improved performance of synthetic sequences is due to a combination of on-target activations and off-target repression.

### Selected Synthetic CREs Drive Desired Tissue-Specific Activity In Vivo

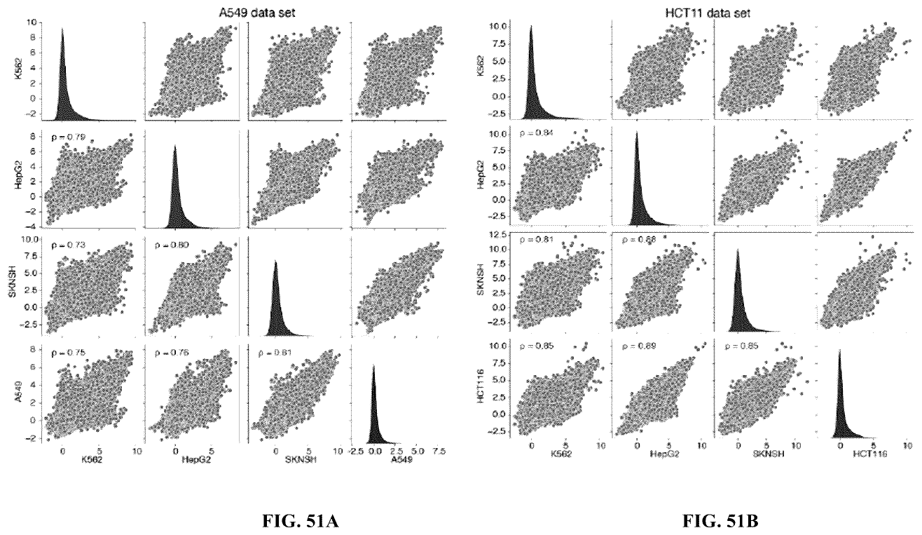

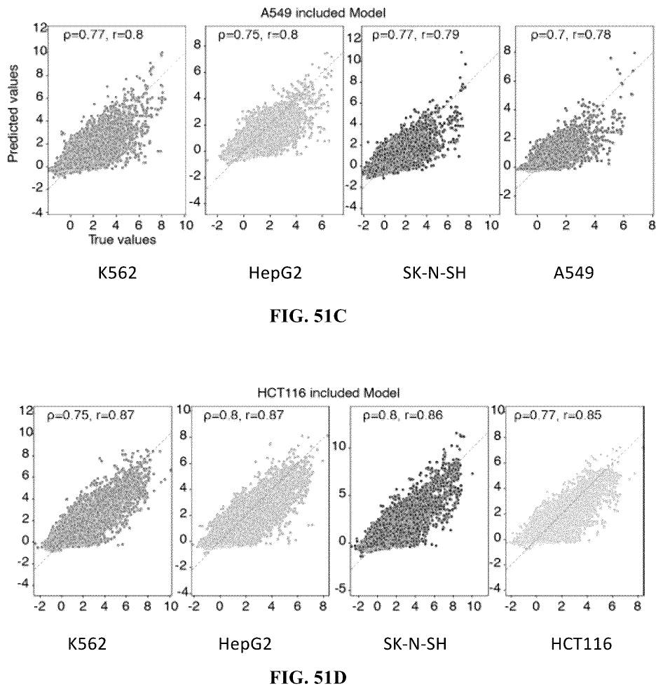

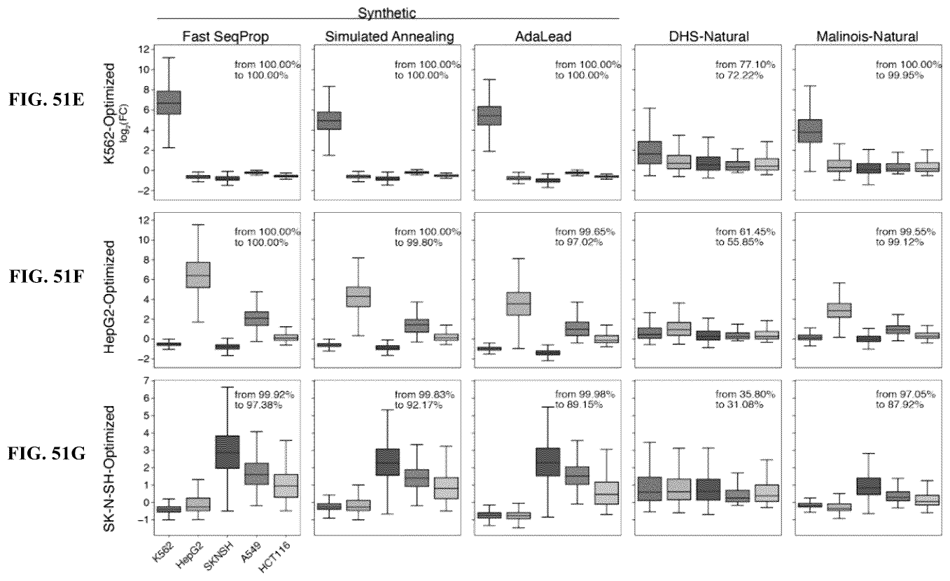

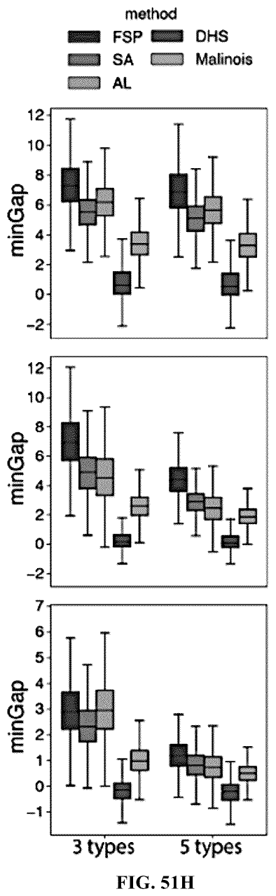

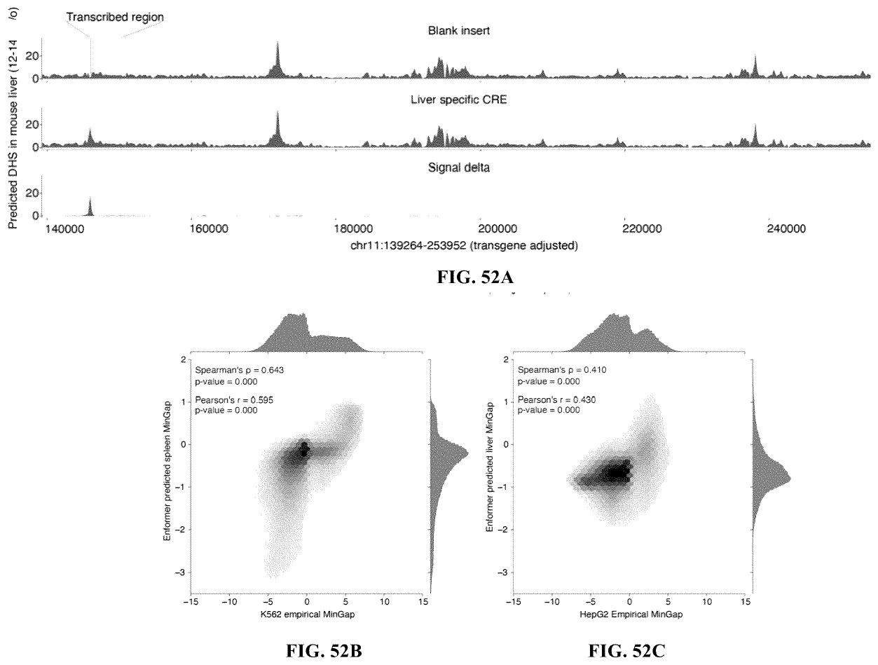

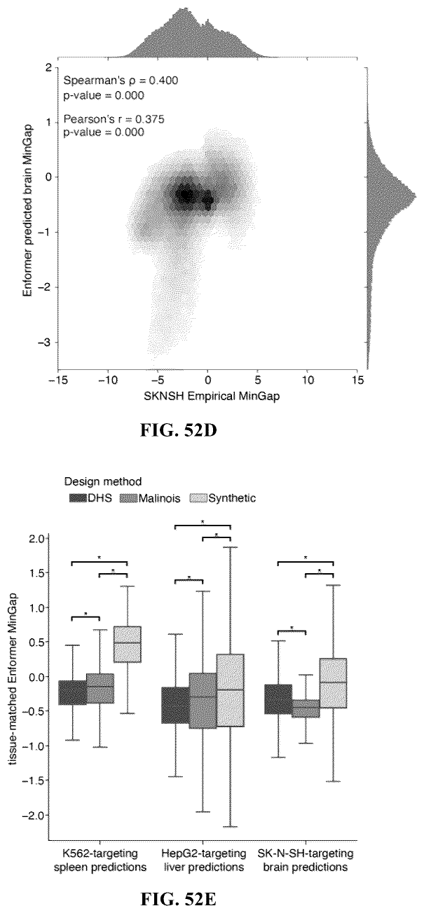

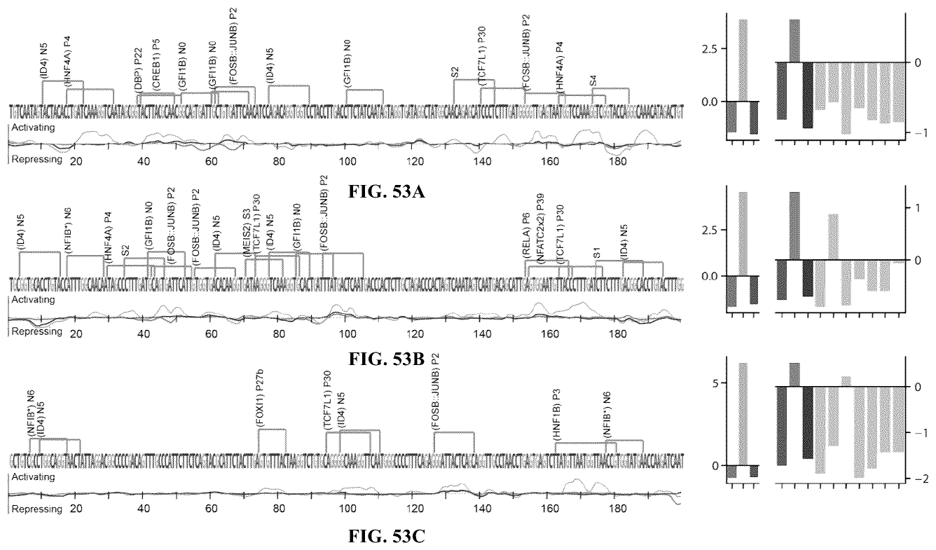

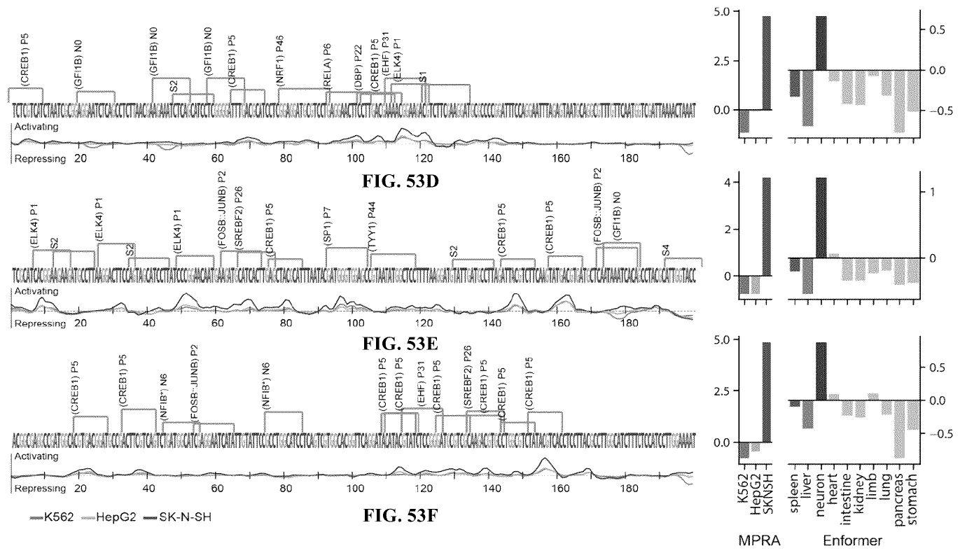

Applicant next sought to assess if the specificity of synthetic CREs would generalize beyond the initial three cell lines used for design. To determine if low off-target activity is maintained in additional cell lines we trained two new CNN models for A549 (lung epithelial cancer; prediction Pearson's r=0.78) and HCT116 (colon epithelial cancer; prediction Pearson's r=0.84) cells, which were not included in the original model used for CODA (FIG. 51A-51D, Methods). Synthetic CREs maintained maximum activity for their target cell type after inclusion of A549 and HCT116, especially those generated using Fast SeqProp (FIG. 51E-51H). To assess specificity of synthetic CREs beyond an episomal reporter context in vitro, Applicant evaluated selected sequences for their ability to drive cell type-specific expression in vivo. Using Enformer, a deep learning model trained on gene regulatory signatures from primary tissues, Applicant predicted the impact of synthetic CREs on epigenetic and transcriptional markers for gene activation (Methods, Supplementary Table 8 of Gosai et al. “Machine-guided design of synthetic cell type-specific cis-regulatory elements” BioRxiv doi: doi.org/10.1101/2023.08.08.552077 (2023), FIG. 52A) 33. Specificity as measured by MPRA in K562, HepG2, and SK-N-SH was significantly correlated with tissue specific Enformer scores in spleen, liver, and neural structures, respectively (FIG. 52B-52D) and was higher in synthetic elements than both groups of natural sequences (FIG. 52E). Encouraged by in vivo specificity of synthetic CREs as measured by in silico approaches, Applicant established a pipeline to nominate and evaluate sequences directly in vertebrate models. Using empirical MPRA results, Malinois contribution scores, in silico predictions of tissue-specific epigenetic signals, and element syntax, we nominated three liver- and three neuronal-specific CREs for in vivo characterization in zebrafish embryos (FIG. 21A, Methods, FIG. 53A-53F).

Applicant inserted synthetic sequences upstream of a minimal promoter driving GFP to emulate the vector design utilized by CODA during in vitro testing85. Applicant injected transposon vectors into embryos and integrated them into the zebrafish genome. To identify the unique expression patterns of each regulatory element, Applicant performed high-resolution, whole-animal imaging at 48 and 96 hours post fertilization for neuronal and liver targets respectively. For sequences designed to drive activity specifically in the liver, 2 of 3 sequences demonstrated strong, consistent expression in the developing liver (FIG. 21B, FIGS. 54A-54B, and FIGS. 55A-55C). Remarkably, Applicant detected minimal off-target expression in non-targeted cell types. Sequences designed for neuronal specificity showed similar success (2 of 3), driving expression in a subset of neuronal cell types (FIG. 21C, FIGS. 56A-56L). For both successful neuronal-nominated CREs, Applicant observed GFP expression within cell bodies and axonal projections of the developing brain and spinal cord (FIG. 21C, FIG. 56H).

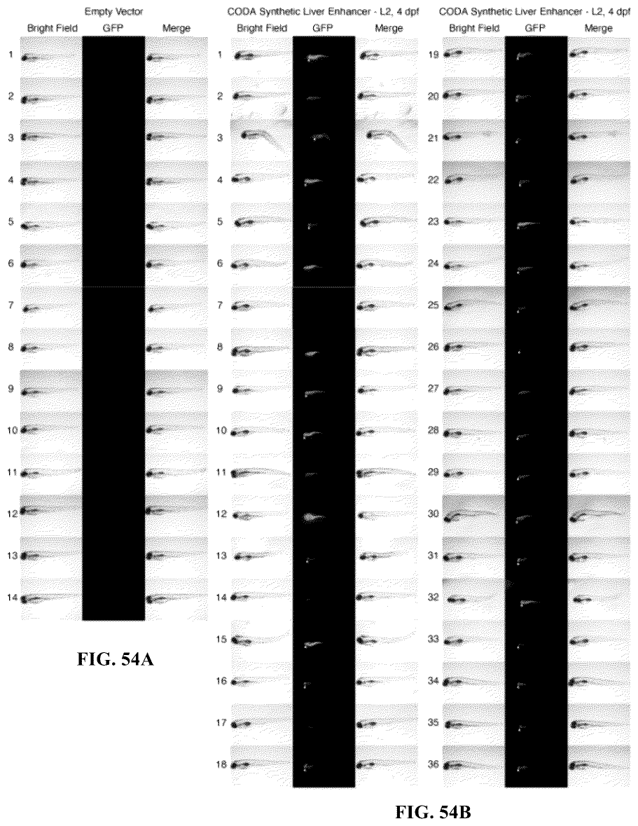

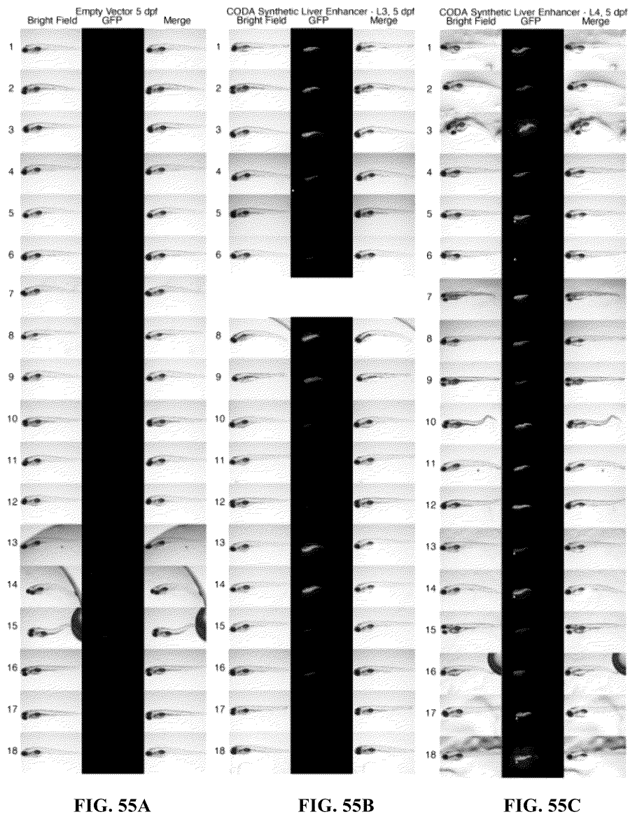

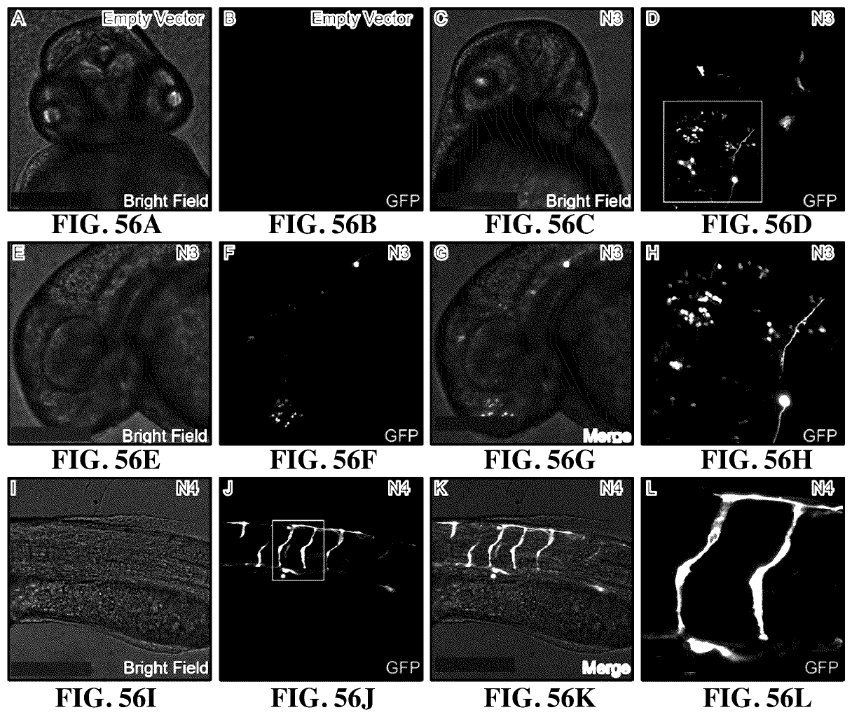

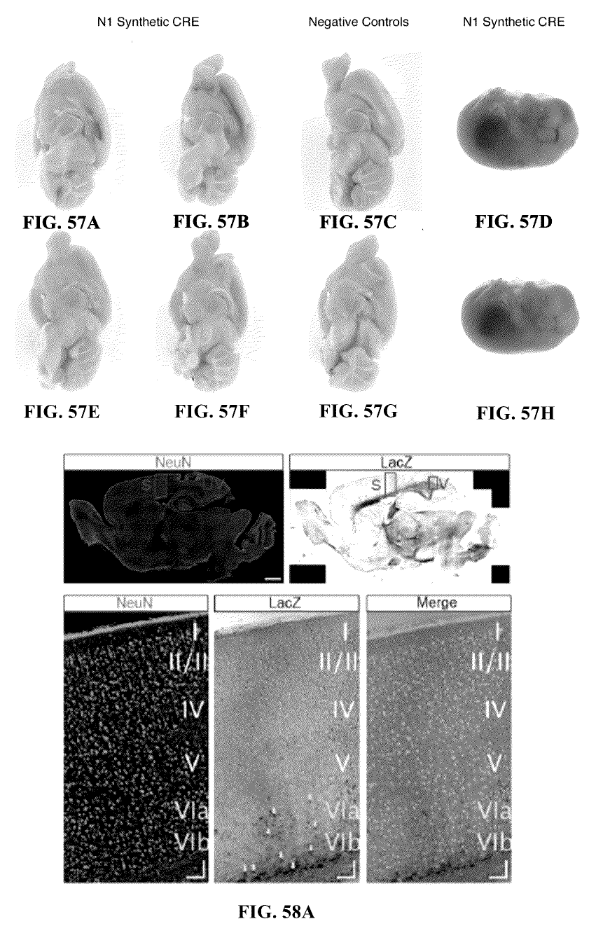

Applicant next evaluated if the activity of the two sequences with neuronal specificity in zebrafish extended to a mammalian mouse model system. Applicant placed each synthetic CRE sequence into a targeting vector upstream of a minimal promoter driving lacZ and GFP, and integrated the construct at the H11 safe harbor locus of the mouse through zygote microinjection86. Applicant harvested embryos at embryonic day 14.5, a time point roughly equivalent to that used in zebrafish, and used lacZ staining to the transgenic embryos to examine expression patterns of the reporter construct driven by the synthetic CRE. Applicant observed specific expression for neuronal #1 (N1) with localized expression in the developing cortex and no additional expression observed elsewhere (FIGS. 57A-57B). To localize the expression patterns further within the cortex, Applicant repeated the reporter assay with the N1 CRE and performed in situ staining of the whole brain at 5 weeks postnatal (FIG. 21D, FIG. 57C-57H). Applicant confirmed cortex specific expression with focal activity occurring in the neurons at neocortical layer 6 and at subplate neurons (FIG. 21E-21G, FIG. 58A-58B).

Having designed and validated a novel CRE with strong neuronal specificity, Applicant sought to further elucidate the factors responsible for transcriptional activity in neuronal cells. Using Malinois' single-nucleotide contributions generated for neuronal N1 in SK-N-SH, Applicant observed two categorically distinct motif classes as contributors to sequence activity: (i) two primary ETS GGA(A/T) binding domains, and (ii) four CREB-like TGACGCA binding domains (FIG. 21H). ETS factors constitute one of the largest transcription factor families, and its members exhibit highly similar binding motifs. Previous work has reported the potential of ETS factors to form heterodimers with CREB87, and Applicant's contribution scores provided support for two heterodimer pairings in the sequence (FIG. 21H, Methods). To assess contribution scores from Malinois Applicant conducted an empirical saturation mutagenesis MPRA in SK-N-SH, which confirmed high-contribution regions and supported motif assignments identified from the contribution scores (FIG. 21H, Methods). In the off-target cell types, contribution scores showed ETS and CREB-like motifs were either reduced or absent, with the presence of two additional negatively contributing motifs, closely matching the repressor GFI1 (FIG. 53D). This suggests that the specificity of neuronal N1 could be partly attributed to the on-target transcriptional activity of cooperative heterodimers and off-target repression by GFI1.

### DISCUSSION

In this study, Applicant developed CODA, an effective strategy to design new synthetic CREs that can direct cell type-specific gene expression by understanding the complex combinatorial rules of cis-regulatory control. CODA builds on previous sequenced-based methods that learned fundamental logics of regulatory grammar to identify cell-type specific CREs from natural or rationally designed sequences^{18, 88-90}, as well as more recent approaches for fully synthetic CREs^{40,41}. This approach is unique in the use of our model Malinois, a direct model of a CRE's transcriptional output in humans, and large-scale testing of synthetic alongside genomic elements which allowed us to directly compare specificity.

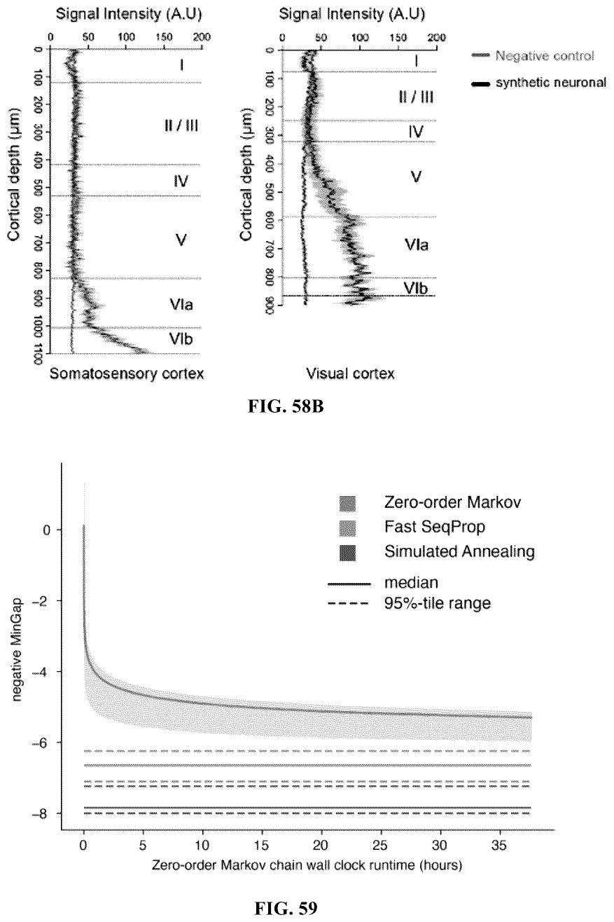

Synthetic sequences designed by CODA easily outperform natural sequences in driving cell type-specific gene expression in a reporter system, which suggests that new functions can be programmed into CREs and interpreted by human cells. Due to the intractability of fully searching sequence space, CODA cannot assuredly identify global specificity maxima, but our exhaustive evaluation of natural sequences demonstrates the design methods we used can identify synthetic sequences that regularly outperform natural ones with 1000-fold greater efficiency compared to previous methods using a zero-order Markov approach (FIG. 59)^{40,41}. By combining high-throughput characterization methods and in vivo reporters, Applicant empirically validated that CODA can efficiently design specific CREs with high success rates, including in mammals.

The dearth of natural sequences capable of achieving exquisite specificity in a desired cell type in this study highlights the difficulty of using human genomic sequences to achieve non-natural objectives for which evolution may not have acted on. Furthermore, DHS elements exhibite both weak on-target activity and poor specificity. This is possibly a reflection of selective pressure that has shaped DHS elements across mammalian evolution to be optimized for redundancy, versatility, and modular function^{91,92}, or alternatively, a weak correlation between quantitative DHS signal and CRE activity. Without human input, CODA deploys unique combinations of strongly on-target activating and off-target repressing TFs within a short sequence that are not commonly found in the human genome, to yield highly specific synthetic CREs. This suggests that Applicant's models have learned a component of the foundational rules governing CREs, and possess the ability to extrapolate this knowledge to unobserved or rarely observed syntax combinations. Future empirical analysis of motif ablation or embedding could be used to further validate how the model interprets regulatory sequences and improve training.

Using Malinois, Applicant were able to identify natural sequences in the genome with moderate proficiency for cell-specific activity, albeit to a lesser degree than synthetics. It was striking that these cell-specific natural sequences represented a broad range of genomic annotations and were less likely to be attributed to known CREs that were found using epigenomic signatures. This highlights the need to carefully consider sequences outside the typically studied candidate CREs when generating libraries with the intent to train high-performance models.

Applicant's high success rate in modeling, generating, and testing sequences in vitro prompted us to extend assessment in vivo. Despite potential challenges of incomplete conservation of tissue types, heterochrony, and lineage-specific regulatory grammar, Applicant's CREs displayed conserved cross-species activity in zebrafish and mice. Applicant's results suggest that CREs designed for tissue-specific targeting can work across species, even in the brain, which has been an ongoing challenge to target with viral-based delivery approaches⁴². An integrated framework leveraging human cell lines in conjunction with whole organism models may thus be a viable approach to rapidly identify CREs to execute novel functions in humans.

Applicant expects that the CODA platform can be extended by integrating additional advancements in deep learning and generative AI, conditioning models on orthogonal data modalities, modeling CRE function in more tissue types, and tasking different biological objectives. While Applicant only tested three cell types here, there is a growing list of clinically actionable tissues that could be benefited, as well as cell types that suffer toxic off-target tropism that could be mitigated by engineered CREs paired with delivery systems. The system here can be applied to these cells based on the exemplary cell systems demonstrated here. Applying MPRA in additional cell types with greater clinical relevance and training new models on these data could enable CODA to better design CREs with specificity tailored for therapeutic applications. As the technology underlying sequence-to-function models continues to evolve, are mechanistically interrogated through ablation studies, and are trained on high-quality MPRA data sets, Applicant expects synthetic element designs to become even more reliable and reduce the experimental burden for in vitro and in vivo validation. With increasingly complex models, it will be essential to determine the bounds of reliable predictions across sequence space to ensure synthetic sequence designs are not based on pathological model predictions.

While Applicant successfully deployed CODA for cell type specificity, the platform is designed to be flexible to any objective function. By combining alternative experimental platforms and models with CODA one could design CREs for drug responsiveness (e.g. glucocorticoids), fine tune expression outputs, or to respond to the complex syntax specific to cancer cells. CODA has improved our ability to write regulatory code tailored to diverse purposes, and could serve as a valuable platform for improving specificity of gene therapies.

### Methods

### Training Malinois, a Model of MPRA Activity of CREs

To enable systematic evaluation of parameters governing data preprocessing, model architecture, and training we developed tools for limited automatic machine learning in PyTorch (github.com/sjgosai/boda2). Applicant implemented support for regression based on DNA sequences using convolutional neural networks. Applicant deployed a containerized application based on this library in conjunction with the Vertex AI platform on Google Cloud to tune all hyperparameters using Bayesian Optimization.

### Data Preprocessing

To construct the train/validation/test dataset to train Malinois, Applicant aggregated the log₂FC output of sequences tested in K562, HepG2, and SK-N-SH from multiple projects (OL indexed reference files in Supplementary Table 1 of Gosai et al. “Machine-guided design of synthetic cell type-specific cis-regulatory elements” BioRxiv doi: doi.org/10.1101/2023.08.08.552077 (2023)). The majority of projects focused on testing the allelic effects of human genetic variation with the remaining projects testing only the reference sequences of the human genome. In total, 776,474 (813,051 before applying filters) unique oligos were aggregated, originating from 10 independent experiments (from three different projects: UKBB [OL27, OL28, OL29, OL30, OL31, OL32, OL33], GTEx [OL41, OL42], OL15). Oligos with a plasmid count less than 20 or no RNA count in any cell type were discarded. The log₂FC of oligos present in more than one UKBB library was averaged across libraries. If an oligo in UKBB was also found in GTEx or OL15, only the UKBB readout was collected and the others were discarded. If an oligo in GTEx (but not in UKBB) was also found in OL15, only the GTEx readout is collected and the OL15 readout was discarded. Non-natural sequences from OL 15 were discarded. Also, oligos with a log₂FC 6 standard deviations below the global mean were discarded (less than 10 oligos). Sequences were padded on both sides with constant sequences from the reporter vector backbone to form 600-bp sequences and converted into one-hot arrays (i.e., A:=[1,0,0,0], C:=[0,1,0,0], G:=[0,0,1,0], T:=[0,0,0,1], N:=[0,0,0,0]). Oligos from chromosomes 19, 21, and X were held out from the parameter training loop as a validation set guide hyperparameter tuning. Oligos from chromosomes 7, 13 were held out from both parameter training and hyperparameter tuning loops as a test set for reporting performance. Data augmentation was performed by including into the training set the reverse complement of the (600-bp) sequences, and duplicating oligos that had a log₂FC greater than 0.5 in any cell type. For locus-specific benchmarking, Applicant aggregated the log₂FC of oligos that tile the GATA1 locus (OL43) following the same counts filtering steps as described above. Applicant generated per-genome-base activity measurements by averaging the MPRA activity of each oligo that overlaps that base pair. Applicant removed oligos genomic coordinates which overlap those in the UKBB and GTEx libraries in scatterplots and correlation calculations. Applicant also aggregated the log₂FC output of 318,247 and 442,482 sequences tested in A549 (OL27, OL28, OL29, OL30, OL31, OL32, OL33) and HCT116 (OL41, OL42), respectively following the same counts filtering steps as described above.

### Model Architecture

The final Malinois model is composed of three functional segments: (1) three convolutional layers with batch normalization and maximum value pooling, (2) a linear layer to integrate positional and feature information from the previous layers, and (3) a stack of branched linear layers such that each output feature is a function of 4 independent transformations. As the first two segments are replicated from the Basset architecture47, Malinois accepts batches of 4×600 arrays corresponding to one-hot encoded DNA sequences, so predictions for 200-nt MPRA oligos are made by padding inputs on both sides with constant sequences from the reporter vector backbone. This strict input sizing requirement ensures hidden states are appropriately shaped when transitioning between segments (1) and (2) of the model. At training initiation weights were initialized using pre-trained weights from a PyTorch implementation of Basset when (1) and (2) were appropriately configured.

### Model Fitting

Applicant trained Malinois using the Vertex AI API on the Google Cloud Platform (GCP). This enabled optimization of all tunable parameters controlling data preprocessing, model architecture, and model training. To do this, Applicant first generated a docker container (gcr.io/sabeti-encode/boda/production: 0.0.11) with an installation of CODA using a GCP VM with the following specifications: Debian based Deep Learning VM for Pytorch CPU/GPU operating system, a2-highgpu-1g machine type, and 1 NVIDIA Tesla A100 40G GPU. The container entrypoint was set to a python script for model training (boda2/src/main.py). Using this container, Applicant deployed Hyperparameter Tuning Jobs using the default algorithm to optimize the indicated hyperparameters (Supplementary Table 7 of Gosai et al. “Machine-guided design of synthetic cell type-specific cis-regulatory elements” BioRxiv doi: doi.org/10.1101/2023.08.08.552077 (2023)). Applicant included a notebook for deploying a Hyperparameter Tuning using Job the Vertex AI SDK (boda2/tutorials/vertex_sdk_launch.ipynb). Applicant finalized model selection for Malinois by benchmarking candidates on the validation set using predictions calculated as described in the next section. All test set benchmarking was retrospective and did not impact decision making in the study. Two additional models were fitted using a subset of sequences tested in either A549 or HCT116 using identical hyperparameter configurations to Malinois.

### Optimization of Cell Specificity

The objective function to guide the sequence design with Simulated Annealing (minimize energy) was the MinGap (Malinois log₂FC prediction in the target cell type minus the maximum off-target cell type log₂FC prediction). The objective function used with the algorithms Fast SeqProp and AdaLead (minimize or maximize respectively) was the bent-MinGap, which is defined as follows. Let y+be the Malinois log₂FC prediction on the target cell type, and y− the maximum of the log₂FC predictions on the off-target cell types of a given sequence (so MinGap=y₊−y₋). We constructed a bending function g(x)=x−e^{-x}+1 to preprocess predictions such that the objective function becomes bent-MinGap=g(y+)−g(y−). We applied g(x) to the predictions to incentivize greater MinGaps with low expression in the off-target cell types. For three generative algorithms to prevent pathologically extreme activity predictions that are common in deep learning methods when computing on sequences highly divergent from the training data, we constrained predictions to a limited interval (default: [−2, 6]) when generating sequences.

### Iterative Maximization of Sequence Function Using Iterative, Generative, and Evolutionary Sequence Generation Algorithms

Fast SeqProp³⁶ was selected as a representative gradient-based local optimization method that exploits the structure of deep learning models to conduct greedy search while retaining the ability to pass true one-hot encoded inputs to the model. Applicant implemented this algorithm as described in previous work but Applicant removed the learnable affine transformation in the instance normalization layer and drew many one-hot encoded samples from the categorical nucleotide probability distribution in each optimization step to more confidently estimate the gradients of the learnable re-parameterized input sequence. The input parameters were randomly initialized (drawn from a normal distribution) and optimized using the Pytorch implementation of the Adam optimization algorithm with a learning rate of 0.5, along with a Cosine Annealing scheduler with a minimum learning rate of 10-6 over 300 training steps. In each training step, the loss function value was the negative average bent-MinGap of 20 sequence samples drawn from the categorical nucleotide probability distribution at that step. Once optimization is finalized, instance normalization is applied to the learned input and 20 sequences were sampled from the obtained distribution, and the sequence with the highest predicted bent-MinGap was collected unless the value was less than 3.6.

AdaLead³⁵, another greedy search algorithm, was selected as a representative evolutionary optimization algorithm for its ease of implementation and previously reported success in DNA sequence optimization. Applicant implemented this algorithm as written in the GitHub repository associated with the original paper. In each run, 20 randomly initialized sequences are optimized over 30 generations with mu=1, recomb_rate=0.1, threshold=0.25, rho=2, using bent-MinGap as the fitness (objective) function. Once optimization is finalized, only the sequence with the highest predicted bent-MinGap is collected unless the MinGap was less than 2. Applicant chose to collect only one sequence per run to maximize diversity in the global batch collected from all runs.

Simulated Annealing⁶⁶ was selected as a representative probabilistic optimization algorithm based on a decades-long history of successful application to a wide range of domains for non-convex optimization. Simulated Annealing starts by jumping between regions with different local optima by occasionally accepting proposals that deteriorate the objective when the sampling temperature is high early in the algorithm. In later stages, the algorithm shifts toward greedy hill climbing as low sampling temperatures only allow proposals that improve the objective to be accepted. Applicant implemented Simulated Annealing based on the Metropolis-Hastings algorithm for Markov Chain Monte Carlo simulations. Proposals were generated symmetrically at each step by mutating 3 random bases. Applicant used negative MinGap (without bending) to simulate the energy landscape of the theoretical system. During optimization the temperature term was reduced using a monotonically decreasing function with a diverging infinite sum (Eq. 1):

$$τ = \frac{1}{1 + {s}^{0.501}} \qquad ( Eq . 1 )$$

To produce sequences with high target-specific activity we used negative MinGap (without bending) to simulate energy of the system.

### Motif Penalization

In order to design a batch of sequences penalizing the enrichment of given motifs in the batch, we introduced to the loss function an additional term explained below. To penalize a single motif of length I, we construct the motif PWM (position-weight matrix, a.k.a. Position-Specific Scoring Matrix, or log probabilities) and use it to score all possible subsequences x of length l in the batch. Let sⱼ=PWM(xⱼ) be the motif score of the subsequence xⱼ, n the number of sequences in the batch, and t a score threshold. Then, the motif penalty is defined as (Eq. 2)

$$\frac{1}{n} {\sum }_{j : {s}_{j} \geq t} {s}_{j} \qquad ( Eq . 2 )$$

where j iterates over all the possible subsequences including their reverse complements. In other words, we sum all the motif scores above the score threshold and divide by the size of the batch. When penalizing m motifs, the term we introduce i s very close to simply averaging the m motif penalties, except that we introduce a weighting factor for each motif penalty to emphasize the penalization of motifs with lower indices (or in our case below, to prioritize motifs based on their order of inclusion to the motif pool). If we let s/=PWM⁽ⁱ⁾(xⱼ) be the motif score of motif i of the subsequence xⱼ, and t⁽ⁱ⁾ the score threshold of motif i, then the total motif penalty given a motif pool {PWM⁽¹⁾, . . . , PWM^{(m)}} is defined as (Eq. 3)

$$\frac{1}{mn} {\sum }_{i ∈ [ m ]} {( m - i + 1 )}^{\frac{1}{3}} {\sum }_{j : {s}_{j}^{( i )} \geq {t}^{( i} )} {s}_{j}^{( i )} \qquad ( Eq . 3 )$$

where the term (m−i+1)^{1/3} is the weighting factor increasing the value of the motif penalties with lower index i.

Applicant used this motif penalty expression to iteratively design sequences subject to an increasing pool of motifs. Applicants call these iterations penalization tracks. A single penalization track starts with the generation of a batch of 500 (non-penalized) sequences, which is then analyzed for motif enrichment (top 10 motifs of length 8 to 15) using STREME via a python wrapper function. Applicant collected the top motif PWM⁽¹⁾ from the analysis and design a second batch of 250 sequences (which we call round-1 penalized sequences) penalizing the motif pool PWM⁽¹⁾}. Then Applicant extracts the top motif PWM⁽¹⁾ enriched in the round-1 penalized sequences and design a third batch of 250 sequences (round-2 penalized sequences) penalizing the motif pool {PWM⁽¹⁾, PWM⁽²⁾}.

Applicant generated 4 penalization tracks for each target cell type, for all three cell types. Applicant defined the score threshold for each motif as a percentage of the motif score of its consensus sequence. The percentages used were 0 for K562-target sequences, and 0.25 for HepG2- and SK-N-SH-target sequences. The reason behind the different choice for K562 is that Applicant found that the optimization process could more easily escape the penalization of GATA by still using suboptimal instances of the motif, so a more stringent penalty was of interest for us. The motivation for using a weighting factor was that Applicant hypothesized that sequence design optimization gravitates more strongly to motifs captured in enrichment analyses of early penalization rounds, so Applicant sought to keep emphasizing the penalization of motifs extracted from earlier rounds.

In FIG. 30B, the motif-presence score (y-axis) of a motif in each sequence was calculated by summing all the motif-match scores that pass the Patser score threshold (as defined in Biopython93), and then dividing by the maximum possible motif score (the match score of the motif consensus sequence).

### K-Mer Analysis

Applicant calculated 4-mer and 7-mer content for sequences in the CODA MPRA library as well as various other sets of reference sequences including 200-mers upstream of RefGene annotated transcription start sites, shuffled CODA sequences, and random 200-mers. Applicant calculated the average Manhattan distance to the k-nearest neighbors distances for 200-mers (k=4) by splitting sequences into groups based on design method, target cell line, and penalty level and using the NearestNeighbors module from scikit-learn (version 1.2.2). Applicant embedded sequences in two-dimensional space based on 4-mer content using the uniform manifold approximation and projection (UMAP) implemented by the umap-learn (version 0.5.2) python package.

### Homology Search Using Nucleotide Blast

Applicant conducted a homology search using NCBI ElasticBLAST to determine if synthetic sequences had measurable homology to any sequences in Nucleotide Collection. Applicant used the blastn algorithm, the dc-megablast task, and a word size of 11 and maintained the defaults for all other settings.

### Selection of Naturally Occurring Cell-Specific Sequences by DNase and Malinois Driven Genome Scan

DHS-natural. To identify CREs broadly replicating across experimental approaches, Applicant first took DNAse peaks from each of the three cell lines (K562, HepG2, and SK-N-SH), and subsetted peaks that intersect with H3K27ac peaks from the same cell type. For the DHS-H3K27ac peaks, in each cell type, we scored the average K562, HepG2, and SK-N-SH DHS signal in the peak. Applicant then calculated the MinGap score for each target cell type using the DHS signal, and selected the 4000 peaks with the largest MinGap score in each cell type.

Malinois-natural. To nominate cell-specific natural sequences with Malinois, we tiled the whole human genome into 200-bp windows using a 50-bp stride and generated predictions for each window sequence. The cell specificity of each sequence was obtained by evaluating the objective function mentioned above (bent-MinGap), and the top 4000 best performing sequences were selected for each cell type.

### Genome Annotation of Natural Sequences

Malinois-natural sequences capture a unique component of the genome compared to
DHS-natural, with 2.7% of Malinois-natural sequences overlapping sequences in our DHS-natural set, and 65.8% residing outside any previously annotated CREs. cCRE BED files for promoter-like sequences, proximal enhancer-like sequences, distal enhancer-like sequences, and CTCF-only were downloaded from the ENCODE SCREEN Portal5 and concatenated into a single BED file for intersection with DHS-natural and Malinois-natural BED files using a custom script. Intersections were done with bedtools 2.30.0 94 and pybedtools 0.9.0 95 with the following command ‘Malinois/DHS-natural BED.intersect (ENCODE_cCRE_BED, wa=True, u=True) and the number of intersections were reported. To determine the genomic features overlapping DHS-natural and Malinois-natural sequences, the same BED files were used as input for ‘annotatePeaks.pl from the homer suite v4.11 96 with the following command’ annotatePeaks.pl inputBED hg38-annStats annStats.txt>annotatePeaksOut.txt′. Annotations for the whole genome (hg38) were generated by dividing the genome into 200-bp intervals using the bedtools makewindows command ‘bedtools makewindows-g hg38.txt-w 200>hg38_200 bp.bed’. Annotations were generated for each cell type (K562, HepG2, SK-N-SH) and sequence selection method (DHS-natural, Malinois-natural.)

### Sampled Integrated Gradients to Compute Contribution Scores of Malinois Predictions

Applicant calculated nucleotide contribution scores for each sequence in the proposed library using an adaptation of the input attribution method Integrated Gradients⁶⁸. Sampled Integrated Gradients considers the expected gradients along the linear path in log-probability space from the background distribution to the distribution that samples the input sequence almost surely. In each point of the linear path, a sequence probability distribution (a.k.a. Position Probability Matrix) is obtained from the log-probability space parameters by applying the Softmax function along the nucleotide axis, and a batch of sequences is sampled from that distribution to be fed into the model. Applicant then calculate the gradients of the batch model predictions with respect to the parameters in the log-probability space, using the straight-through estimator to backpropagate through the sampling operation. The batch gradients are averaged for each point in the path and approximate the gradient integral as in the original formulation of the method. In this case, the subtraction of the baseline input from the input of interest involves the parameters in log-probability space. This adaptation of Integrated Gradients provides two useful features. First, the sequence inputs being fed to the model are always in one-hot form, avoiding evaluations of inputs thatoff the vertices of the simplex on which the model was trained which could more easily lead to pathological predictions. Second, the original method relies on choosing an appropriate single baseline input against which to compare the input of interest which might not always be straight forward, whereas our adaptation uses a background distribution of sequences as the baseline. Favorably, when choosing the uniform background (0.25, 0.25, 0.25, 0.25), the parameters in log-probability space where the line path is traversed become the zero matrix, which removes the need to subtract the baseline from the input of interest. Applicant can then more easily extract integrated gradients for all tokens in all positions (by omitting masking the gradients with the one-hot input), which we found useful as hypothetical scores for TF-MoDISco.

### Contribution Block Ablation

To test the value of contribution scores obtained with Sampled Integrated Gradients, Applicant conducted an in silico ablation study of the library sequences using contribution blocks (to be defined below) to randomize segments of the sequences. The goal of the study was to investigate the predicted log2FoldChange effects of randomizing positions within the sequences corresponding to blocks of either positive or negative contribution, or random positions outside blocks. The result of the study is summarized in FIG. 42A-42F. Overall, randomizing segments of the sequences associated with negative contribution resulted in an increase of predicted activity in either the target or off-target cell type, while randomizing those associated with positive contribution completely destroyed the activity in the target cell type, and marginally decreased the (already repressed) activity in off-target cell types. In order to make calls of contribution blocks in any given sequence, Applicant took the 200 contribution scores and built a smoothed contribution signal using a ID Gaussian Filter (scipy.ndimage.gaussian_filterld) with a sigma of 1.15. Applicant defined a positive contribution block whenever the smoothed signal was above a threshold of 0.015 for 4 contiguous positions or more, and negative whenever it was below 0.015 for 4 contiguous positions or more. Outside positions were those not assigned to a contribution block. For each target cell type group (25,000 sequences), contribution block calls and ablations were performed for all three prediction tasks. For example, taking the K562-target sequences, three different ablations and call sets were carried out: (i) block calls using contribution scores in K562 assessing the K562 activity effect (target cell type), (ii) block calls using contribution scores in HepG2 assessing the HepG2 activity effect (off-target cell type), and block calls using contribution scores in SKNSH assessing the SKNSH activity effect (off-target cell type). This resulted in a total of 9 sets of calls and ablations. When assessing the effect of disrupting positions outside contribution blocks, we subsampled the outside coverage (number of positions not in blocks) to match the upper half of the distribution of coverage sizes of positive and negative contribution blocks together, whenever possible. For the SK-N-SH-target group, for example, such a distribution match was not possible since the total number of available positions from which to sample was simply not large enough globally. The same was true for the target cell type outside ablation in K562 and HepG2, which might be expected since positive contribution blocks alone have large coverages. Applicant performed this outside subsampling to have comparable ablation sizes across categories, but also because disrupting all the positions outside blocks that have low coverage (resulting in very high outside coverages) introduces too much noise into the sequence when most of the sequence is disrupted. Applicant set a minimum of 5 positions to be disrupted by outside coverages.

### Propeller Plots

A propeller dot plot (top row of FIG. 19E) is a 2-dimensional plot scheme of our own device which seeks to elucidate the cross-dimensional non-uniformity of 3-dimensional points. In this coordinate system, a point's radial distance from the origin corresponds to the difference between the maximum and minimum values. Its deviant angle from the axis corresponding to the maximum value quantifies the position of the median value within the range of the minimum and maximum values. Namely, the angle is proportional to the ratio between two differences: (i) the difference of the median and minimum values, and (ii) the difference of the maximum and minimum values. This ratio represents the 60-degree-angle fraction deviating from the axis corresponding to the maximum value towards the axis corresponding to the median value. A higher angle of deviation (maximum of 60 degrees) indicates that the median value is closer to the maximum value, while a lower angle (minimum of 0 degrees) of deviation indicates that the median value is closer to the minimum value.

This can also be formulated in terms of the MinGap (maximum-median) and MaxGap (maximum-minimum). In our coordinate system, the MaxGap corresponds to the radial distance. The difference (1-MinGap/MaxGap) corresponds to the 60-degree-angle fraction deviating from the axis corresponding to the maximum value towards the axis corresponding to the median value. The MinGap: MaxGap ratio controls how much a point gravitates toward a main axis and away from the in-between-axis areas. A ratio of 0 means that the MinGap is zero and therefore the median value is equal to the maximum, so the point will be exactly between two axes. If the ratio is 1, it means that the median and the minimum values are equal, therefore the point will fall exactly in the axis corresponding to the maximum value. Note that, in order for this point of view to work with target and off-target cell type activities, we assume that the maximum cell type activity is the intended target cell type. This implies that, when counting sequences that pass specificity thresholds in FIG. 19E, some sequences get their target cell type reassigned to the cell type with the maximum activity, with DHS-natural sequences being the group that most benefits from the reassignment. A total of 652 sequences pass the lenient specificity threshold of MaxGap>1 and MinGap/MaxGap>0.5 by getting their target cell type reassigned (DHS-natural: 565, Malinois-natural: 39, AdaLead: 12, Simulated Annealing: 5, Fast SeqProp: 0, Fast SeqProp penalized: 4). However, only 16 sequences pass the stringent specificity threshold of MaxGap>4 and MinGap/MaxGap>0.5 by getting their target cell type reassigned (DHS-natural: 15, Malinois-natural: 0, AdaLead: 1, Simulated Annealing: 0, Fast SeqProp: 0, Fast SeqProp penalized: 0).

As an example of coordinate calculation, take the point (5, 3, 1). This point would have a radial distance of 5−1=4 and an angle of deviation from the axis of the first dimension of (3−1)/(5−1) * (60 deg)=30 deg (in the direction of the axis of the second dimension). In terms of the MinGap: MaxGap ratio, the angle of deviation from the axis of the first dimension (the dimension of the maximum value) towards the axis of the second dimension would be (1−(5−3)/(5−1)*(60 deg)=30 deg. Observe that all the points of the form (x+4, x+2, x), for any real value of x, will have the same coordinates as the point (5, 3, 1).

A propeller count plot (bottom row of FIG. 19E) shows the percentage of points that fall in each given area of a propeller dot plot. The teal, yellow, and red regions capture sequences in which the median value is closer to the minimum value than to the maximum value. The two synthetic groups in FIG. 19E were randomly subsampled to have exactly 12,000 sequences each and avoid over-plotting compared to the plots of the two natural groups. FIG. 40 shows the complete propeller plots broken down by design method.

Oligos with a replicate log₂FC standard error greater than 1 in any cell type were omitted from the plots.

### Motif Discovery

Applicant used TF-MoDISco Lite^{69,70} to extract sequence motifs to be predicted as functional by Malinois through contribution scores obtained through Sampled Integrated Gradients (SIG). As described above, SIG naturally provides hypothetical contribution scores (as defined by TF-MoDISco) when selecting the uniform random background by simply carrying out the equivalent of the full process minus masking out using the input sequence one-hot matrix. The final contribution scores can then be retrieved masking out the hypothetical contribution using the input sequence one-hot matrices, as required by TF-MoDISco. Applicant computed hypothetical contribution scores for each of the three prediction tasks and ran TF-MoDISco Lite with 100,000 seqlets and a window size of 200 (equivalent results were obtained using 1,000,000 seqlets). Applicant aggregated the discovered patterns across prediction tasks following their provided example using modiscolite.aggregator.SimilarPatternsCollapser. TF-MoDISco Lite results are provided as positive and negative patterns.

### TF-MoDISco Patterns to PWMs

To convert a TF-MoDISco positive pattern living in the hypothetical-contribution-score space into a Position-Weight Matrix (PWM), Applicant divided the pattern scores by the maximum position score sum and multiplied by 10. To obtain the Position-Probability Matrix (PPM) Applicant applied the Softmax function to each position vector. Some of our TF-MoDISco negative patterns are a combination of a negative pattern (negative contributions) and a positive one (positive contributions). Thus, in order to convert a TF-MoDISco negative pattern into a PWM, Applicant first reversed the sign directionality of the negative portions (as informed by the pattern scores living in contribution-score space, not hypothetical) and compensated their magnitude by multiplying by 1.2 (because our negative contribution scores are in general smaller in magnitude than positive ones perhaps due to the nature of the training data target distribution that has a positive bias). Then, Applicant proceeded as with the positive patterns.

### Core Motifs (TF-MoDISco)

Since TF-MoDISco, in addition to capturing isolated ungapped motifs, is able to capture patterns that are combinations of motifs, Applicant heuristically extracted core ungapped patterns that, to varying degrees, account for all the combinations observed in the TF-MoDISco merged results. To manually define the starts and stops of core motifs, Applicant relied on scoring the full pattern PWMs against themselves using TOMTOM97, information content contours, and visual examination. The core motif IDs are derived from the IDs of the original patterns from which they were extracted. To convert the patterns into PWMs and PPMs, we applied the same operations as described above. Matches to human known TF binding motifs were assigned using TOMTOM with default parameters against the databases JASPAR CORE (2022)⁷¹ and HOCOMOCO Human (v11 FULL) ⁷².

### Core Motifs (STREME)

In addition to extracting sequence motifs with TF-MoDISco, Applicant also performed a motif enrichment analysis using STREME. First, to assess the agreement between a given STREME motif and its predicted functionality as measured by contribution scores, Applicant weighted-averaged the hypothetical contribution scores corresponding to all the sequence segments determined to be a match to the motif (as provided by FIMO with default parameters, using motif scores as weights), and compared the score averages (one set of averages per each prediction task) to the motif's Information-Content Matrix (ICM). Applicant refers to the weighted average hypothetical scores as the “contribution-score” projection. All motifs with overall positive contribution scores that had a strong agreement with their contribution-score projection had been already captured by TF-MoDISco, suggesting that the TF-MoDISco positive pattern results are very comprehensive. However, Applicant found a small number of STREME motifs with negative contribution scores that had a strong agreement with their contribution-score projection, so Applicant decided to include them to the list of core motifs. It is worth noting that these motifs had negative contribution scores with moderate-to-low magnitude. Applicant speculated that the reason TF-MoDISco might not have been able to detect them is because the contribution allocated in the seqlets that would correspond to these motifs too often falls below the threshold of the distribution of negative scores, making it hard to discriminate them from noise or insignificant scores. Running TF-MoDISco with 1M seqlets did not change the results. Applicant retrieved 11 such STREME motifs with strong agreement with their contribution-score projection not captured by TF-MoDISco, 9 of which were clustered together into 3 groups with nearly identical contribution-score projection (up to 1 or 2 additional positions to the left or right). This gave us a total of 5 STREME negative patterns in contribution-score projection form that were included to the list of core motifs. Their conversion to PWM and PPM forms followed the same process as with the TF-MoDISco patterns. Matches to human known TF binding motifs were assigned using TOMTOM with default parameters against the databases JASPAR CORE (2022)⁷¹ and HOCOMOCO Human (v11 FULL)⁷².

### Contribution Score-Based Motif Hit Mapping

To find instances of the core motifs present in the CODA sequence library, Applicant leveraged the hypothetical contribution scores of the sequences to match sequence segments to the core motifs in hypothetical-contribution-score form. First, we padded with zeros left and right all the sequence hypothetical contribution scores, yielding a matrix of dimensions 3×75000×4×210. Second, for a core motif of length l, Applicant computed all the Pearson correlation coefficients between every possible subsequence hypothetical contribution scores of length l (matrices of size 75000×4× l) and the core motif's hypothetical contribution scores in forward and reverse complement orientations. For each cell type dimension, Applicant randomly sampled 500,000 Pearson correlation coefficients (arising from a single core motif) to obtain the value min (0. 75, μ+4σ) to serve as a coefficient threshold, where μ, σ represent the mean and the standard deviation, respectively, of the subsampled distribution. All subsequences for which their hypothetical contribution scores scored above their coefficient threshold were collected as motif hits for the given core motif. Applicant repeated this process for all core motifs across all cell types.

### Motifs Embedded in Random Background

Applicant embedded single motifs in random sequences to measure their standalone predicted effect compared to fully random sequences. For each motif, Applicant built a 200×4 Position-Probability Matrix (PPM) consisting of the motif's PPM in the middle and random background ([0.25, 0.25, 0.25, 0.25]) everywhere else. Applicant sampled 5000 sequences from it and fed them to Malinois to obtain predictions in each cell type. Applicant also sampled 5000 sequences from a 200×4 PPM of uniform background everywhere (no motif in the middle), and fed them to Malinois to serve as baseline.

### Motif Ablation

Applicant sought to assess the predicted effect of disrupting all instances of a single motif in Applicant's sequence library. For each motif, Applicant collected the particular batch of sequences that had at least one instance of such motif, replaced all the instances with random segments (sampled from uniform background), and fed them to Malinois to obtain predictions in each cell type. Applicant performed this step 5 times, averaged the 5 predictions of each disrupted sequence, and subtracted from the average the batch's original predicted activities to obtain the predicted disrupting effect. For example, say that a sequence has one instance of a given motif in positions 20-32. Applicant inserted a random sequence segment in those positions and got the disrupted sequence's predictions. We did this 5 times, so 5 different random segments (with 5 different predictions) in positions 20-32, and averaged the 5 predictions (to mildly marginalize potential effects of replacing with random segments). The disrupting effect would be this average prediction minus the sequence's original predicted activity. Applicant aggregated the disrupting effects by motif presence (as defined above in the last paragraph of motif penalization in this section). To find instances of core motifs, Applicant used the contribution score-based motif hit mapping described above. To find instances of the original TF-MoDISco patterns, Applicant used FIMO (with the default parameters), since our contribution score-based motif hit mapping might not handle gapped patterns as well as FIMO. When submitting the pattern PPMs to FIMO, Applicant trimmed the patterns at both ends such that the start/stop of the pattern is the first/last position to have an information content of at least 0.15 bits.

### Motif Contributions

To get a motif's overall contribution, we performed a weighted average of the contribution score sums contained in all the motif instances provided by our motif hit method across the three prediction tasks. The average was weighted using the motif scores corresponding to the Pearson correlation coefficients mentioned above. The overall regulatory directionality of a motif (activator or repressor) is given by the sign of the mean of the weighted averages across cell types. For all motifs, the overall regulatory directionality agrees with the original TF-MoDISco designation as a positive or negative pattern.

### Motif Co-Occurrence

Applicant says a pair of motifs co-occur whenever a sequence has at least one instance of each motif. By co-occurrence percentage of a motif pair Applicant means the percentage of sequences in a given group in which the motif pair co-occurs.

### Non-Negative Matrix Factorization Analysis of Motif Programs

Applicant used non-negative matrix factorization (NMF) to model semantic relationships between motifs in our sequence library (scikit-learn version 1.2.2, initialized with NNDSVD AR, Frobenius loss). First Applicant counted motif matches in each sequence with the contribution score-based motif hit mapping described above98 to generate where rows represent sequences in the library and columns correspond to motifs. The sample matrix X can then be decomposed into the coefficients and features matrices and, respectively. Applicant tested decomposing sequences into k∈[8,28] programs using bi-cross-validation99 and identified an “elbow” in the reconstruction error at k=1214 (data not shown). For when plotting the coefficient matrix comparative analysis, we normalize the coefficient matrix such that the rows to sum to 1. Applicant quantified the function of each decomposed program by calculating a weighted average of motif contributions (see Methods subsection: Motif contributions above) for each program using the motif weights in the features matrix. Motif contributions were clipped to an upper bound of 3 to mitigate the impact of extreme outliers.

### MPRA Saturation Mutagenesis Plot

The saturation mutagenesis study (Table 11) of the sequence in FIG. 21G consisted in empirically testing the activity of all the possible 600 variants of the sequence (3 variants per position, 200 positions). Applicant followed an identical protocol to the previous MPRAs in SK-N-SH with this saturation mutagenesis library. Applicant visualized the effect of each variant as the subtraction of the activity of the original sequence from each variant-sequence's activity, resulting in the lollipops in FIG. 21H. The mean variant effect is represented in the height of the logo sequence letters but in the opposite direction.

TABLE 11

| ID | sat_mut | log2FoldChange | lfcSE | celltype |
|---|---|---|---|---|
| 20211212_75659_621411_391::fsp_sknsh_0 | m0 | 5.071070921 | 0.16452305 | sknsh |
| 20211212_75659_621411_391::fsp_sknsh_0:mA107C | mA107C | 3.801058599 | 0.05206037 | sknsh |
| 20211212_75659_621411_391::fsp_sknsh_0:mA107G | mA107G | 3.821344042 | 0.05627328 | sknsh |
| 20211212_75659_621411_391::fsp_sknsh_0:mA107T | mA107T | 4.198405081 | 0.04836139 | sknsh |
| 20211212_75659_621411_391::fsp_sknsh_0:mA110C | mA110C | 5.406644179 | 0.04754692 | sknsh |
| 20211212_75659_621411_391::fsp_sknsh_0:mA110G | mA110G | 4.83917943 | 0.05048339 | sknsh |
| 20211212_75659_621411_391::fsp_sknsh_0:mA110T | mA110T | 5.531245895 | 0.04691714 | sknsh |
| 20211212_75659_621411_391::fsp_sknsh_0:mA111C | mA111C | 4.464740852 | 0.05254641 | sknsh |
| 20211212_75659_621411_391::fsp_sknsh_0:mA111G | mA111G | 3.566544572 | 0.05385883 | sknsh |
| 20211212_75659_621411_391::fsp_sknsh_0:mA111T | mA111T | 3.503878103 | 0.04961137 | sknsh |
| 20211212_75659_621411_391::fsp_sknsh_0:mA112C | mA112C | 3.762780786 | 0.046879 | sknsh |
| 20211212_75659_621411_391::fsp_sknsh_0:mA112G | mA112G | 3.738844966 | 0.06174608 | sknsh |
| 20211212_75659_621411_391::fsp_sknsh_0:mA112T | mA112T | 4.098763526 | 0.05566272 | sknsh |
| 20211212_75659_621411_391::fsp_sknsh_0:mA113C | mA113C | 5.979884187 | 0.05029184 | sknsh |
| 20211212_75659_621411_391::fsp_sknsh_0:mA113G | mA113G | 6.408982715 | 0.04648689 | sknsh |
| 20211212_75659_621411_391::fsp_sknsh_0:mA113T | mA113T | 3.573925128 | 0.05705898 | sknsh |
| 20211212_75659_621411_391::fsp_sknsh_0:mA117C | mA117C | 1.760961835 | 0.08189524 | sknsh |
| 20211212_75659_621411_391::fsp_sknsh_0:mA117G | mA117G | 1.550612507 | 0.07283672 | sknsh |
| 20211212_75659_621411_391::fsp_sknsh_0:mA117T | mA117T | 1.30743711 | 0.08672812 | sknsh |
| 20211212_75659_621411_391::fsp_sknsh_0:mA118C | mA118C | 1.455198552 | 0.0652866 | sknsh |
| 20211212_75659_621411_391::fsp_sknsh_0:mA118G | mA118G | 1.587687678 | 0.08003853 | sknsh |
| 20211212_75659_621411_391::fsp_sknsh_0:mA118T | mA118T | 3.943841826 | 0.04672204 | sknsh |
| 20211212_75659_621411_391::fsp_sknsh_0:mA120C | mA120C | 5.591083561 | 0.04704007 | sknsh |
| 20211212_75659_621411_391::fsp_sknsh_0:mA120G | mA120G | 4.896127628 | 0.05010297 | sknsh |
| 20211212_75659_621411_391::fsp_sknsh_0:mA120T | mA120T | 6.166467592 | 0.04661521 | sknsh |
| 20211212_75659_621411_391::fsp_sknsh_0:mA129C | mA129C | 5.681960896 | 0.04880471 | sknsh |
| 20211212_75659_621411_391::fsp_sknsh_0:mA129G | mA129G | 6.161445786 | 0.05078104 | sknsh |
| 20211212_75659_621411_391::fsp_sknsh_0:mA129T | mA129T | 5.606024981 | 0.05400939 | sknsh |
| 20211212_75659_621411_391::fsp_sknsh_0:mA12C | mA12C | 5.35487844 | 0.05325765 | sknsh |
| 20211212_75659_621411_391::fsp_sknsh_0:mA12G | mA12G | 5.067520857 | 0.05177678 | sknsh |
| 20211212_75659_621411_391::fsp_sknsh_0:mA12T | mA12T | 5.629088293 | 0.05682092 | sknsh |
| 20211212_75659_621411_391::fsp_sknsh_0:mA130C | mA130C | 4.630031329 | 0.05932171 | sknsh |
| 20211212_75659_621411_391::fsp_sknsh_0:mA130G | mA130G | 4.932022026 | 0.04884801 | sknsh |
| 20211212_75659_621411_391::fsp_sknsh_0:mA130T | mA130T | 4.993503004 | 0.04779409 | sknsh |
| 20211212_75659_621411_391::fsp_sknsh_0:mA133C | mA133C | 5.348174042 | 0.05019479 | sknsh |
| 20211212_75659_621411_391::fsp_sknsh_0:mA133G | mA133G | 5.438554848 | 0.05389028 | sknsh |
| 20211212_75659_621411_391::fsp_sknsh_0:mA133T | mA133T | 5.214873964 | 0.04759135 | sknsh |
| 20211212_75659_621411_391::fsp_sknsh_0:mA13C | mA13C | 5.051045324 | 0.05337468 | sknsh |
| 20211212_75659_621411_391::fsp_sknsh_0:mA13G | mA13G | 5.007983916 | 0.05010452 | sknsh |
| 20211212_75659_621411_391::fsp_sknsh_0:mA13T | mA13T | 5.004172563 | 0.0434321 | sknsh |
| 20211212_75659_621411_391::fsp_sknsh_0:mA144C | mA144C | 4.825675323 | 0.05244857 | sknsh |
| 20211212_75659_621411_391::fsp_sknsh_0:mA144G | mA144G | 5.059622603 | 0.04986405 | sknsh |
| 20211212_75659_621411_391::fsp_sknsh_0:mA144T | mA144T | 4.816240986 | 0.04792876 | sknsh |
| 20211212_75659_621411_391::fsp_sknsh_0:mA150C | mA150C | 5.624811198 | 0.045927 | sknsh |
| 20211212_75659_621411_391::fsp_sknsh_0:mA150G | mA150G | 7.006894881 | 0.04594957 | sknsh |
| 20211212_75659_621411_391::fsp_sknsh_0:mA150T | mA150T | 5.660539678 | 0.0485742 | sknsh |
| 20211212_75659_621411_391::fsp_sknsh_0:mA153C | mA153C | 5.491268587 | 0.04983468 | sknsh |
| 20211212_75659_621411_391::fsp_sknsh_0:mA153G | mA153G | 5.288834126 | 0.04752418 | sknsh |
| 20211212_75659_621411_391::fsp_sknsh_0:mA153T | mA153T | 5.432409778 | 0.04589729 | sknsh |
| 20211212_75659_621411_391::fsp_sknsh_0:mA154C | mA154C | 5.410752157 | 0.05002978 | sknsh |
| 20211212_75659_621411_391::fsp_sknsh_0:mA154G | mA154G | 5.230542723 | 0.15571542 | sknsh |
| 20211212_75659_621411_391::fsp_sknsh_0:mA154T | mA154T | 5.208463948 | 0.40279742 | sknsh |
| 20211212_75659_621411_391::fsp_sknsh_0:mA158C | mA158C | 4.996647313 | 0.05248285 | sknsh |
| 20211212_75659_621411_391::fsp_sknsh_0:mA158G | mA158G | 4.993356545 | 0.04593987 | sknsh |
| 20211212_75659_621411_391::fsp_sknsh_0:mA158T | mA158T | 5.025730591 | 0.04678247 | sknsh |
| 20211212_75659_621411_391::fsp_sknsh_0:mA160C | mA160C | 5.21740664 | 0.06953725 | sknsh |
| 20211212_75659_621411_391::fsp_sknsh_0:mA160G | mA160G | 4.840774572 | 0.05369668 | sknsh |
| 20211212_75659_621411_391::fsp_sknsh_0:mA160T | mA160T | 4.810358775 | 0.05088828 | sknsh |
| 20211212_75659_621411_391::fsp_sknsh_0:mA163C | mA163C | 5.299199641 | 0.0497119 | sknsh |
| 20211212_75659_621411_391::fsp_sknsh_0:mA163G | mA163G | 5.139912945 | 0.05018709 | sknsh |
| 20211212_75659_621411_391::fsp_sknsh_0:mA163T | mA163T | 4.985231791 | 0.04664913 | sknsh |
| 20211212_75659_621411_391::fsp_sknsh_0:mA164C | mA164C | 5.057745436 | 0.04802616 | sknsh |
| 20211212_75659_621411_391::fsp_sknsh_0:mA164G | mA164G | 5.080189378 | 0.04570854 | sknsh |
| 20211212_75659_621411_391::fsp_sknsh_0:mA164T | mA164T | 4.902129443 | 0.05480827 | sknsh |
| 20211212_75659_621411_391::fsp_sknsh_0:mA168C | mA168C | 5.131413486 | 0.04603165 | sknsh |
| 20211212_75659_621411_391::fsp_sknsh_0:mA168G | mA168G | 5.022343379 | 0.04589874 | sknsh |
| 20211212_75659_621411_391::fsp_sknsh_0:mA168T | mA168T | 4.846928963 | 0.04823318 | sknsh |
| 20211212_75659_621411_391::fsp_sknsh_0:mA180C | mA180C | 5.094106155 | 0.05190643 | sknsh |
| 20211212_75659_621411_391::fsp_sknsh_0:mA180G | mA180G | 4.550568391 | 0.05267733 | sknsh |
| 20211212_75659_621411_391::fsp_sknsh_0:mA180T | mA180T | 5.040456404 | 0.05062254 | sknsh |
| 20211212_75659_621411_391::fsp_sknsh_0:mA181C | mA181C | 5.137170805 | 0.05141102 | sknsh |
| 20211212_75659_621411_391::fsp_sknsh_0:mA181G | mA181G | 5.063395029 | 0.04963271 | sknsh |
| 20211212_75659_621411_391::fsp_sknsh_0:mA181T | mA181T | 5.670803465 | 0.04458383 | sknsh |
| 20211212_75659_621411_391::fsp_sknsh_0:mA188C | mA188C | 5.099936294 | 0.04341855 | sknsh |
| 20211212_75659_621411_391::fsp_sknsh_0:mA188G | mA188G | 5.026227051 | 0.04640098 | sknsh |
| 20211212_75659_621411_391::fsp_sknsh_0:mA188T | mA188T | 5.045443113 | 0.04907824 | sknsh |
| 20211212_75659_621411_391::fsp_sknsh_0:mA191C | mA191C | 5.096671826 | 0.04618176 | sknsh |
| 20211212_75659_621411_391::fsp_sknsh_0:mA191G | mA191G | 5.142033733 | 0.04892737 | sknsh |
| 20211212_75659_621411_391::fsp_sknsh_0:mA191T | mA191T | 4.968712029 | 0.04651551 | sknsh |
| 20211212_75659_621411_391::fsp_sknsh_0:mA192C | mA192C | 5.169637456 | 0.05204425 | sknsh |
| 20211212_75659_621411_391::fsp_sknsh_0:mA192G | mA192G | 5.034568697 | 0.05563467 | sknsh |
| 20211212_75659_621411_391::fsp_sknsh_0:mA192T | mA192T | 5.061263934 | 0.04957076 | sknsh |
| 20211212_75659_621411_391::fsp_sknsh_0:mA193C | mA193C | 4.975119388 | 0.04878102 | sknsh |
| 20211212_75659_621411_391::fsp_sknsh_0:mA193G | mA193G | 5.117395148 | 0.0496161 | sknsh |
| 20211212_75659_621411_391::fsp_sknsh_0:mA193T | mA193T | 4.908564883 | 0.04626499 | sknsh |
| 20211212_75659_621411_391::fsp_sknsh_0:mA194C | mA194C | 4.71150257 | 0.36500118 | sknsh |
| 20211212_75659_621411_391::fsp_sknsh_0:mA194G | mA194G | 5.132982937 | 0.05083032 | sknsh |
| 20211212_75659_621411_391::fsp_sknsh_0:mA194T | mA194T | 5.136926503 | 0.16621487 | sknsh |
| 20211212_75659_621411_391::fsp_sknsh_0:mA197C | mA197C | 4.992435077 | 0.05130971 | sknsh |
| 20211212_75659_621411_391::fsp_sknsh_0:mA197G | mA197G | 4.976220774 | 0.28962852 | sknsh |
| 20211212_75659_621411_391::fsp_sknsh_0:mA197T | mA197T | 4.910931897 | 0.04762544 | sknsh |
| 20211212_75659_621411_391::fsp_sknsh_0:mA198C | mA198C | 4.140204633 | 0.20823749 | sknsh |
| 20211212_75659_621411_391::fsp_sknsh_0:mA198G | mA198G | 5.084098891 | 0.22374342 | sknsh |
| 20211212_75659_621411_391::fsp_sknsh_0:mA198T | mA198T | 2.234624443 | 3.1607391 | sknsh |
| 20211212_75659_621411_391::fsp_sknsh_0:mA199C | mA199C | 4.815920896 | 0.19126195 | sknsh |
| 20211212_75659_621411_391::fsp_sknsh_0:mA199G | mA199G | 5.196917635 | 0.19861559 | sknsh |
| 20211212_75659_621411_391::fsp_sknsh_0:mA199T | mA199T | 5.698254622 | 0.41849892 | sknsh |
| 20211212_75659_621411_391::fsp_sknsh_0:mA20C | mA20C | 5.146390227 | 0.05380903 | sknsh |
| 20211212_75659_621411_391::fsp_sknsh_0:mA20G | mA20G | 4.595694657 | 0.04805055 | sknsh |
| 20211212_75659_621411_391::fsp_sknsh_0:mA20T | mA20T | 4.712908759 | 0.04736352 | sknsh |
| 20211212_75659_621411_391::fsp_sknsh_0:mA23C | mA23C | 4.799334222 | 0.04796855 | sknsh |
| 20211212_75659_621411_391::fsp_sknsh_0:mA23G | mA23G | 4.733757174 | 0.05124779 | sknsh |
| 20211212_75659_621411_391::fsp_sknsh_0:mA23T | mA23T | 4.717552043 | 0.05128658 | sknsh |
| 20211212_75659_621411_391::fsp_sknsh_0:mA24C | mA24C | 4.679352264 | 0.0534486 | sknsh |
| 20211212_75659_621411_391::fsp_sknsh_0:mA24G | mA24G | 4.806565811 | 0.05432204 | sknsh |
| 20211212_75659_621411_391::fsp_sknsh_0:mA24T | mA24T | 4.664366683 | 0.05186475 | sknsh |
| 20211212_75659_621411_391::fsp_sknsh_0:mA29C | mA29C | 5.702315315 | 0.05302726 | sknsh |
| 20211212_75659_621411_391::fsp_sknsh_0:mA29G | mA29G | 4.946612013 | 0.05014932 | sknsh |
| 20211212_75659_621411_391::fsp_sknsh_0:mA29T | mA29T | 4.879408212 | 0.05237647 | sknsh |
| 20211212_75659_621411_391::fsp_sknsh_0:mA37C | mA37C | 5.121150454 | 0.05203106 | sknsh |
| 20211212_75659_621411_391::fsp_sknsh_0:mA37G | mA37G | 4.99928984 | 0.04950041 | sknsh |
| 20211212_75659_621411_391::fsp_sknsh_0:mA37T | mA37T | 5.14312616 | 0.04893923 | sknsh |
| 20211212_75659_621411_391::fsp_sknsh_0:mA38C | mA38C | 4.906412072 | 0.05427173 | sknsh |
| 20211212_75659_621411_391::fsp_sknsh_0:mA38G | mA38G | 5.187964401 | 0.04685243 | sknsh |
| 20211212_75659_621411_391::fsp_sknsh_0:mA38T | mA38T | 4.660842096 | 0.05439704 | sknsh |
| 20211212_75659_621411_391::fsp_sknsh_0:mA41C | mA41C | 5.312756878 | 0.04995481 | sknsh |
| 20211212_75659_621411_391::fsp_sknsh_0:mA41G | mA41G | 5.103587638 | 0.05388598 | sknsh |
| 20211212_75659_621411_391::fsp_sknsh_0:mA41T | mA41T | 5.261592847 | 0.0559283 | sknsh |
| 20211212_75659_621411_391::fsp_sknsh_0:mA42C | mA42C | 5.274428968 | 0.05093992 | sknsh |
| 20211212_75659_621411_391::fsp_sknsh_0:mA42G | mA42G | 5.169684047 | 0.05086177 | sknsh |
| 20211212_75659_621411_391::fsp_sknsh_0:mA42T | mA42T | 5.237903244 | 0.04701355 | sknsh |
| 20211212_75659_621411_391::fsp_sknsh_0:mA44C | mA44C | 5.122259016 | 0.04990389 | sknsh |
| 20211212_75659_621411_391::fsp_sknsh_0:mA44G | mA44G | 4.92477926 | 0.17298518 | sknsh |
| 20211212_75659_621411_391::fsp_sknsh_0:mA44T | mA44T | 4.952406708 | 0.04990936 | sknsh |
| 20211212_75659_621411_391::fsp_sknsh_0:mA45C | mA45C | 4.897123534 | 0.05236983 | sknsh |
| 20211212_75659_621411_391::fsp_sknsh_0:mA45G | mA45G | 5.507929077 | 0.04643123 | sknsh |
| 20211212_75659_621411_391::fsp_sknsh_0:mA45T | mA45T | 4.863144998 | 0.05165277 | sknsh |
| 20211212_75659_621411_391::fsp_sknsh_0:mA46C | mA46C | 5.097130261 | 0.05012514 | sknsh |
| 20211212_75659_621411_391::fsp_sknsh_0:mA46G | mA46G | 5.013300916 | 0.05260428 | sknsh |
| 20211212_75659_621411_391::fsp_sknsh_0:mA46T | mA46T | 5.093740685 | 0.05323517 | sknsh |
| 20211212_75659_621411_391::fsp_sknsh_0:mA51C | mA51C | 5.176986114 | 0.0537424 | sknsh |
| 20211212_75659_621411_391::fsp_sknsh_0:mA51G | mA51G | 5.498381862 | 0.05000677 | sknsh |
| 20211212_75659_621411_391::fsp_sknsh_0:mA51T | mA51T | 5.125108752 | 0.04602407 | sknsh |
| 20211212_75659_621411_391::fsp_sknsh_0:mA54C | mA54C | 5.387565487 | 0.04804636 | sknsh |
| 20211212_75659_621411_391::fsp_sknsh_0:mA54G | mA54G | 5.301861638 | 0.04886586 | sknsh |
| 20211212_75659_621411_391::fsp_sknsh_0:mA54T | mA54T | 5.357057283 | 0.04899076 | sknsh |
| 20211212_75659_621411_391::fsp_sknsh_0:mA64C | mA64C | 5.127479515 | 0.05021385 | sknsh |
| 20211212_75659_621411_391::fsp_sknsh_0:mA64G | mA64G | 5.190130202 | 0.0470517 | sknsh |
| 20211212_75659_621411_391::fsp_sknsh_0:mA64T | mA64T | 5.218831703 | 0.04720115 | sknsh |
| 20211212_75659_621411_391::fsp_sknsh_0:mA69C | mA69C | 4.192597446 | 0.05891807 | sknsh |
| 20211212_75659_621411_391::fsp_sknsh_0:mA69G | mA69G | 4.561690275 | 0.04891904 | sknsh |
| 20211212_75659_621411_391::fsp_sknsh_0:mA69T | mA69T | 3.922652645 | 0.05449283 | sknsh |
| 20211212_75659_621411_391::fsp_sknsh_0:mA73C | mA73C | 3.446816044 | 0.04884218 | sknsh |
| 20211212_75659_621411_391::fsp_sknsh_0:mA73G | mA73G | 4.470681263 | 0.04918209 | sknsh |
| 20211212_75659_621411_391::fsp_sknsh_0:mA73T | mA73T | 4.268910434 | 0.05256148 | sknsh |
| 20211212_75659_621411_391::fsp_sknsh_0:mA81C | mA81C | 5.558274562 | 0.0450022 | sknsh |
| 20211212_75659_621411_391::fsp_sknsh_0:mA81G | mA81G | 3.918355179 | 0.04662144 | sknsh |
| 20211212_75659_621411_391::fsp_sknsh_0:mA81T | mA81T | 4.475827493 | 0.04887868 | sknsh |
| 20211212_75659_621411_391::fsp_sknsh_0:mA84C | mA84C | 5.183904762 | 0.0521072 | sknsh |
| 20211212_75659_621411_391::fsp_sknsh_0:mA84G | mA84G | 4.463927364 | 0.05153879 | sknsh |
| 20211212_75659_621411_391::fsp_sknsh_0:mA84T | mA84T | 4.860381937 | 0.05384162 | sknsh |
| 20211212_75659_621411_391::fsp_sknsh_0:mA8C | mA8C | 4.80299597 | 0.05535714 | sknsh |
| 20211212_75659_621411_391::fsp_sknsh_0:mA8G | mA8G | 4.500994082 | 0.05350304 | sknsh |
| 20211212_75659_621411_391::fsp_sknsh_0:mA8T | mA8T | 4.830515272 | 0.24807046 | sknsh |
| 20211212_75659_621411_391::fsp_sknsh_0:mA94C | mA94C | 5.347204426 | 0.05308041 | sknsh |
| 20211212_75659_621411_391::fsp_sknsh_0:mA94G | mA94G | 4.681381384 | 0.05041156 | sknsh |
| 20211212_75659_621411_391::fsp_sknsh_0:mA94T | mA94T | 4.556110356 | 0.05242688 | sknsh |
| 20211212_75659_621411_391::fsp_sknsh_0:mA97C | mA97C | 5.51827806 | 0.04661324 | sknsh |
| 20211212_75659_621411_391::fsp_sknsh_0:mA97G | mA97G | 4.64728433 | 0.0497048 | sknsh |
| 20211212_75659_621411_391::fsp_sknsh_0:mA97T | mA97T | 5.477226575 | 0.04862679 | sknsh |
| 20211212_75659_621411_391::fsp_sknsh_0:mA98C | mA98C | 2.669808317 | 0.0489046 | sknsh |
| 20211212_75659_621411_391::fsp_sknsh_0:mA98G | mA98G | 3.662621199 | 0.04905342 | sknsh |
| 20211212_75659_621411_391::fsp_sknsh_0:mA98T | mA98T | 2.97272935 | 0.05339521 | sknsh |
| 20211212_75659_621411_391::fsp_sknsh_0:mC102A | mC102A | 2.546953667 | 0.06170143 | sknsh |
| 20211212_75659_621411_391::fsp_sknsh_0:mC102G | mC102G | 3.231645135 | 0.04713284 | sknsh |
| 20211212_75659_621411_391::fsp_sknsh_0:mC102T | mC102T | 2.879199523 | 0.05374829 | sknsh |
| 20211212_75659_621411_391::fsp_sknsh_0:mC103A | mC103A | 3.289264653 | 0.04756933 | sknsh |
| 20211212_75659_621411_391::fsp_sknsh_0:mC103G | mC103G | 3.563975711 | 0.04608872 | sknsh |
| 20211212_75659_621411_391::fsp_sknsh_0:mC103T | mC103T | 3.401700217 | 0.05233133 | sknsh |
| 20211212_75659_621411_391::fsp_sknsh_0:mC108A | mC108A | 4.075696123 | 0.05571151 | sknsh |
| 20211212_75659_621411_391::fsp_sknsh_0:mC108G | mC108G | 3.339572879 | 0.05493554 | sknsh |
| 20211212_75659_621411_391::fsp_sknsh_0:mC108T | mC108T | 4.117564824 | 0.06160169 | sknsh |
| 20211212_75659_621411_391::fsp_sknsh_0:mC10A | mC10A | 5.027150562 | 0.04880333 | sknsh |
| 20211212_75659_621411_391::fsp_sknsh_0:mC10G | mC10G | 5.121063303 | 0.05070539 | sknsh |
| 20211212_75659_621411_391::fsp_sknsh_0:mC10T | mC10T | 4.878473865 | 0.05027398 | sknsh |
| 20211212_75659_621411_391::fsp_sknsh_0:mC114A | mC114A | 2.756251439 | 0.05946436 | sknsh |
| 20211212_75659_621411_391::fsp_sknsh_0:mC114G | mC114G | 2.060066317 | 0.07169616 | sknsh |
| 20211212_75659_621411_391::fsp_sknsh_0:mC114T | mC114T | 2.317197177 | 0.06913216 | sknsh |
| 20211212_75659_621411_391::fsp_sknsh_0:mC121A | mC121A | 4.627106527 | 0.05517583 | sknsh |
| 20211212_75659_621411_391::fsp_sknsh_0:mC121G | mC121G | 4.669294776 | 0.0501191 | sknsh |
| 20211212_75659_621411_391::fsp_sknsh_0:mC121T | mC121T | 3.832788201 | 0.04947818 | sknsh |
| 20211212_75659_621411_391::fsp_sknsh_0:mC124A | mC124A | 5.114624754 | 0.04988736 | sknsh |
| 20211212_75659_621411_391::fsp_sknsh_0:mC124G | mC124G | 5.123231267 | 0.04942028 | sknsh |
| 20211212_75659_621411_391::fsp_sknsh_0:mC124T | mC124T | 5.15630168 | 0.05052896 | sknsh |
| 20211212_75659_621411_391::fsp_sknsh_0:mC127A | mC127A | 5.587680638 | 0.0558699 | sknsh |
| 20211212_75659_621411_391::fsp_sknsh_0:mC127G | mC127G | 5.435051529 | 0.05533987 | sknsh |
| 20211212_75659_621411_391::fsp_sknsh_0:mC127T | mC127T | 5.451002812 | 0.05287237 | sknsh |
| 20211212_75659_621411_391::fsp_sknsh_0:mC136A | mC136A | 5.132131064 | 0.04980248 | sknsh |
| 20211212_75659_621411_391::fsp_sknsh_0:mC136G | mC136G | 5.080181644 | 0.04915253 | sknsh |
| 20211212_75659_621411_391::fsp_sknsh_0:mC136T | mC136T | 5.292708256 | 0.04524648 | sknsh |
| 20211212_75659_621411_391::fsp_sknsh_0:mC138A | mC138A | 4.960080506 | 0.04876288 | sknsh |
| 20211212_75659_621411_391::fsp_sknsh_0:mC138G | mC138G | 4.804356419 | 0.05251189 | sknsh |
| 20211212_75659_621411_391::fsp_sknsh_0:mC138T | mC138T | 4.928158634 | 0.04959942 | sknsh |
| 20211212_75659_621411_391::fsp_sknsh_0:mC139A | mC139A | 4.840986985 | 0.042204 | sknsh |
| 20211212_75659_621411_391::fsp_sknsh_0:mC139G | mC139G | 4.665596737 | 0.05381121 | sknsh |
| 20211212_75659_621411_391::fsp_sknsh_0:mC139T | mC139T | 4.653525507 | 0.05292332 | sknsh |
| 20211212_75659_621411_391::fsp_sknsh_0:mC140A | mC140A | 4.946970235 | 0.05044145 | sknsh |
| 20211212_75659_621411_391::fsp_sknsh_0:mC140G | mC140G | 5.107124899 | 0.04870306 | sknsh |
| 20211212_75659_621411_391::fsp_sknsh_0:mC140T | mC140T | 4.854710153 | 0.04685663 | sknsh |
| 20211212_75659_621411_391::fsp_sknsh_0:mC141A | mC141A | 4.812268631 | 0.05280416 | sknsh |
| 20211212_75659_621411_391::fsp_sknsh_0:mC141G | mC141G | 4.960800128 | 0.04594982 | sknsh |
| 20211212_75659_621411_391::fsp_sknsh_0:mC141T | mC141T | 4.871059389 | 0.04809242 | sknsh |
| 20211212_75659_621411_391::fsp_sknsh_0:mC148A | mC148A | 5.33980835 | 0.04884905 | sknsh |
| 20211212_75659_621411_391::fsp_sknsh_0:mC148G | mC148G | 5.299019844 | 0.05221407 | sknsh |
| 20211212_75659_621411_391::fsp_sknsh_0:mC148T | mC148T | 4.889869646 | 0.04803471 | sknsh |
| 20211212_75659_621411_391::fsp_sknsh_0:mC149A | mC149A | 4.826148358 | 0.05646656 | sknsh |
| 20211212_75659_621411_391::fsp_sknsh_0:mC149G | mC149G | 4.083257981 | 0.05921337 | sknsh |
| 20211212_75659_621411_391::fsp_sknsh_0:mC149T | mC149T | 4.156283387 | 0.05089836 | sknsh |
| 20211212_75659_621411_391::fsp_sknsh_0:mC15A | mC15A | 4.634270146 | 0.05182635 | sknsh |
| 20211212_75659_621411_391::fsp_sknsh_0:mC15G | mC15G | 4.720095066 | 0.05223465 | sknsh |
| 20211212_75659_621411_391::fsp_sknsh_0:mC15T | mC15T | 4.666596609 | 0.05324782 | sknsh |
| 20211212_75659_621411_391::fsp_sknsh_0:mC167A | mC167A | 4.717244583 | 0.0527155 | sknsh |
| 20211212_75659_621411_391::fsp_sknsh_0:mC167G | mC167G | 5.370814636 | 0.04665724 | sknsh |
| 20211212_75659_621411_391::fsp_sknsh_0:mC167T | mC167T | 4.711944566 | 0.04807293 | sknsh |
| 20211212_75659_621411_391::fsp_sknsh_0:mC171A | mC171A | 4.7619877 | 0.04901078 | sknsh |
| 20211212_75659_621411_391::fsp_sknsh_0:mC171G | mC171G | 4.82720019 | 0.05068723 | sknsh |
| 20211212_75659_621411_391::fsp_sknsh_0:mC171T | mC171T | 4.093669588 | 0.05467967 | sknsh |
| 20211212_75659_621411_391::fsp_sknsh_0:mC179A | mC179A | 5.027868342 | 0.05271844 | sknsh |
| 20211212_75659_621411_391::fsp_sknsh_0:mC179G | mC179G | 4.979413323 | 0.04980879 | sknsh |
| 20211212_75659_621411_391::fsp_sknsh_0:mC179T | mC179T | 4.981484532 | 0.04819719 | sknsh |
| 20211212_75659_621411_391::fsp_sknsh_0:mC17A | mC17A | 4.453137923 | 0.05523259 | sknsh |
| 20211212_75659_621411_391::fsp_sknsh_0:mC17G | mC17G | 4.643052196 | 0.05519633 | sknsh |
| 20211212_75659_621411_391::fsp_sknsh_0:mC17T | mC17T | 4.54880268 | 0.04892366 | sknsh |
| 20211212_75659_621411_391::fsp_sknsh_0:mC186A | mC186A | 4.946151224 | 0.04494804 | sknsh |
| 20211212_75659_621411_391::fsp_sknsh_0:mC186G | mC186G | 5.140550053 | 0.05103032 | sknsh |
| 20211212_75659_621411_391::fsp_sknsh_0:mC186T | mC186T | 4.797121415 | 0.0501182 | sknsh |
| 20211212_75659_621411_391::fsp_sknsh_0:mC195A | mC195A | 4.86334775 | 0.05743639 | sknsh |
| 20211212_75659_621411_391::fsp_sknsh_0:mC195G | mC195G | 4.861203119 | 0.05036687 | sknsh |
| 20211212_75659_621411_391::fsp_sknsh_0:mC195T | mC195T | 5.214083158 | 0.20146131 | sknsh |
| 20211212_75659_621411_391::fsp_sknsh_0:mC26A | mC26A | 5.028709764 | 0.04716835 | sknsh |
| 20211212_75659_621411_391::fsp_sknsh_0:mC26G | mC26G | 4.723321898 | 0.04841036 | sknsh |
| 20211212_75659_621411_391::fsp_sknsh_0:mC26T | mC26T | 4.954900061 | 0.0542461 | sknsh |
| 20211212_75659_621411_391::fsp_sknsh_0:mC28A | mC28A | 4.874052747 | 0.04786883 | sknsh |
| 20211212_75659_621411_391::fsp_sknsh_0:mC28G | mC28G | 5.033091917 | 0.04977788 | sknsh |
| 20211212_75659_621411_391::fsp_sknsh_0:mC28T | mC28T | 4.865132556 | 0.04893091 | sknsh |
| 20211212_75659_621411_391::fsp_sknsh_0:mC2A | mC2A | 5.14484045 | 0.04974203 | sknsh |
| 20211212_75659_621411_391::fsp_sknsh_0:mC2G | mC2G | 5.633822216 | 0.05355652 | sknsh |
| 20211212_75659_621411_391::fsp_sknsh_0:mC2T | mC2T | 5.682470796 | 0.05124243 | sknsh |
| 20211212_75659_621411_391::fsp_sknsh_0:mC31A | mC31A | 4.843436528 | 0.04975239 | sknsh |
| 20211212_75659_621411_391::fsp_sknsh_0:mC31G | mC31G | 4.826838621 | 0.04689197 | sknsh |
| 20211212_75659_621411_391::fsp_sknsh_0:mC31T | mC31T | 4.785311115 | 0.05330682 | sknsh |
| 20211212_75659_621411_391::fsp_sknsh_0:mC32A | mC32A | 4.406576711 | 0.04943877 | sknsh |
| 20211212_75659_621411_391::fsp_sknsh_0:mC32G | mC32G | 4.925352706 | 0.04781672 | sknsh |
| 20211212_75659_621411_391::fsp_sknsh_0:mC32T | mC32T | 4.732956307 | 0.0547475 | sknsh |
| 20211212_75659_621411_391::fsp_sknsh_0:mC34A | mC34A | 6.165226698 | 0.0498326 | sknsh |
| 20211212_75659_621411_391::fsp_sknsh_0:mC34G | mC34G | 5.067146202 | 0.05011359 | sknsh |
| 20211212_75659_621411_391::fsp_sknsh_0:mC34T | mC34T | 4.856363471 | 0.05302901 | sknsh |
| 20211212_75659_621411_391::fsp_sknsh_0:mC39A | mC39A | 5.120420003 | 0.04628552 | sknsh |
| 20211212_75659_621411_391::fsp_sknsh_0:mC39G | mC39G | 5.155163526 | 0.05146915 | sknsh |
| 20211212_75659_621411_391::fsp_sknsh_0:mC39T | mC39T | 4.641722652 | 0.04859311 | sknsh |
| 20211212_75659_621411_391::fsp_sknsh_0:mC48A | mC48A | 4.989781872 | 0.05095711 | sknsh |
| 20211212_75659_621411_391::fsp_sknsh_0:mC48G | mC48G | 4.850412561 | 0.05072476 | sknsh |
| 20211212_75659_621411_391::fsp_sknsh_0:mC48T | mC48T | 4.923764144 | 0.05094092 | sknsh |
| 20211212_75659_621411_391::fsp_sknsh_0:mC4A | mC4A | 4.523163588 | 0.05117722 | sknsh |
| 20211212_75659_621411_391::fsp_sknsh_0:mC4G | mC4G | 4.545728211 | 0.331864 | sknsh |
| 20211212_75659_621411_391::fsp_sknsh_0:mC4T | mC4T | 5.079157539 | 0.24119478 | sknsh |
| 20211212_75659_621411_391::fsp_sknsh_0:mC50A | mC50A | 4.943940681 | 0.04839714 | sknsh |
| 20211212_75659_621411_391::fsp_sknsh_0:mC50G | mC50G | 5.66130645 | 0.04486496 | sknsh |
| 20211212_75659_621411_391::fsp_sknsh_0:mC50T | mC50T | 4.852787292 | 0.05988482 | sknsh |
| 20211212_75659_621411_391::fsp_sknsh_0:mC53A | mC53A | 5.14565636 | 0.04964772 | sknsh |
| 20211212_75659_621411_391::fsp_sknsh_0:mC53G | mC53G | 5.168874214 | 0.04566955 | sknsh |
| 20211212_75659_621411_391::fsp_sknsh_0:mC53T | mC53T | 5.113415204 | 0.04783286 | sknsh |
| 20211212_75659_621411_391::fsp_sknsh_0:mC56A | mC56A | 5.51130413 | 0.04827158 | sknsh |
| 20211212_75659_621411_391::fsp_sknsh_0:mC56G | mC56G | 5.060079708 | 0.05103246 | sknsh |
| 20211212_75659_621411_391::fsp_sknsh_0:mC56T | mC56T | 5.521164781 | 0.05102474 | sknsh |
| 20211212_75659_621411_391::fsp_sknsh_0:mC57A | mC57A | 5.384472759 | 0.05028643 | sknsh |
| 20211212_75659_621411_391::fsp_sknsh_0:mC57G | mC57G | 4.853284068 | 0.04765934 | sknsh |
| 20211212_75659_621411_391::fsp_sknsh_0:mC57T | mC57T | 5.007522851 | 0.05336779 | sknsh |
| 20211212_75659_621411_391::fsp_sknsh_0:mC59A | mC59A | 5.112374239 | 0.04952708 | sknsh |
| 20211212_75659_621411_391::fsp_sknsh_0:mC59G | mC59G | 5.247989893 | 0.05060867 | sknsh |
| 20211212_75659_621411_391::fsp_sknsh_0:mC59T | mC59T | 4.973849214 | 0.04774661 | sknsh |
| 20211212_75659_621411_391::fsp_sknsh_0:mC70A | mC70A | 3.506328543 | 0.05560972 | sknsh |
| 20211212_75659_621411_391::fsp_sknsh_0:mC70G | mC70G | 3.623854502 | 0.05173036 | sknsh |
| 20211212_75659_621411_391::fsp_sknsh_0:mC70T | mC70T | 4.136088435 | 0.05339058 | sknsh |
| 20211212_75659_621411_391::fsp_sknsh_0:mC72A | mC72A | 5.025593495 | 0.04878394 | sknsh |
| 20211212_75659_621411_391::fsp_sknsh_0:mC72G | mC72G | 3.78367105 | 0.04603298 | sknsh |
| 20211212_75659_621411_391::fsp_sknsh_0:mC72T | mC72T | 5.226363195 | 0.04899206 | sknsh |
| 20211212_75659_621411_391::fsp_sknsh_0:mC75A | mC75A | 5.419219305 | 0.04737326 | sknsh |
| 20211212_75659_621411_391::fsp_sknsh_0:mC75G | mC75G | 6.371190939 | 0.04757731 | sknsh |
| 20211212_75659_621411_391::fsp_sknsh_0:mC75T | mC75T | 4.972101426 | 0.05038805 | sknsh |
| 20211212_75659_621411_391::fsp_sknsh_0:mC76A | mC76A | 5.110894025 | 0.04532713 | sknsh |
| 20211212_75659_621411_391::fsp_sknsh_0:mC76G | mC76G | 5.042224822 | 0.04499454 | sknsh |
| 20211212_75659_621411_391::fsp_sknsh_0:mC76T | mC76T | 4.761283969 | 0.04961844 | sknsh |
| 20211212_75659_621411_391::fsp_sknsh_0:mC78A | mC78A | 4.357232638 | 0.04595692 | sknsh |
| 20211212_75659_621411_391::fsp_sknsh_0:mC78G | mC78G | 4.675320781 | 0.05118424 | sknsh |
| 20211212_75659_621411_391::fsp_sknsh_0:mC78T | mC78T | 4.513354397 | 0.04934105 | sknsh |
| 20211212_75659_621411_391::fsp_sknsh_0:mC7A | mC7A | 4.814353215 | 0.17704686 | sknsh |
| 20211212_75659_621411_391::fsp_sknsh_0:mC7G | mC7G | 5.278067463 | 0.04672512 | sknsh |
| 20211212_75659_621411_391::fsp_sknsh_0:mC7T | mC7T | 4.544659789 | 0.32918676 | sknsh |
| 20211212_75659_621411_391::fsp_sknsh_0:mC87A | mC87A | 3.991173506 | 0.04862659 | sknsh |
| 20211212_75659_621411_391::fsp_sknsh_0:mC87G | mC87G | 3.825993132 | 0.05595834 | sknsh |
| 20211212_75659_621411_391::fsp_sknsh_0:mC87T | mC87T | 4.432933858 | 0.0492735 | sknsh |
| 20211212_75659_621411_391::fsp_sknsh_0:mC90A | mC90A | 6.041503797 | 0.04809264 | sknsh |
| 20211212_75659_621411_391::fsp_sknsh_0:mC90G | mC90G | 4.755855546 | 0.05173558 | sknsh |
| 20211212_75659_621411_391::fsp_sknsh_0:mC90T | mC90T | 4.540293315 | 0.05544715 | sknsh |
| 20211212_75659_621411_391::fsp_sknsh_0:mC91A | mC91A | 6.099096961 | 0.04594866 | sknsh |
| 20211212_75659_621411_391::fsp_sknsh_0:mC91G | mC91G | 5.52075085 | 0.04830336 | sknsh |
| 20211212_75659_621411_391::fsp_sknsh_0:mC91T | mC91T | 4.864565725 | 0.0488413 | sknsh |
| 20211212_75659_621411_391::fsp_sknsh_0:mC99A | mC99A | 2.993322457 | 0.05281403 | sknsh |
| 20211212_75659_621411_391::fsp_sknsh_0:mC99G | mC99G | 4.850794507 | 0.05771427 | sknsh |
| 20211212_75659_621411_391::fsp_sknsh_0:mC99T | mC99T | 3.588851668 | 0.05065987 | sknsh |
| 20211212_75659_621411_391::fsp_sknsh_0:mG106A | mG106A | 4.403749293 | 0.05486375 | sknsh |
| 20211212_75659_621411_391::fsp_sknsh_0:mG106C | mG106C | 4.867521803 | 0.05011395 | sknsh |
| 20211212_75659_621411_391::fsp_sknsh_0:mG106T | mG106T | 6.04327902 | 0.05250398 | sknsh |
| 20211212_75659_621411_391::fsp_sknsh_0:mG109A | mG109A | 3.464006325 | 0.04776751 | sknsh |
| 20211212_75659_621411_391::fsp_sknsh_0:mG109C | mG109C | 3.594043176 | 0.06142384 | sknsh |
| 20211212_75659_621411_391::fsp_sknsh_0:mG109T | mG109T | 3.864692184 | 0.05546199 | sknsh |
| 20211212_75659_621411_391::fsp_sknsh_0:mG115A | mG115A | 1.495166577 | 0.07258129 | sknsh |
| 20211212_75659_621411_391::fsp_sknsh_0:mG115C | mG115C | 1.331912271 | 0.07202787 | sknsh |
| 20211212_75659_621411_391::fsp_sknsh_0:mG115T | mG115T | 1.594851983 | 0.0674065 | sknsh |
| 20211212_75659_621411_391::fsp_sknsh_0:mG116A | mG116A | 2.87519374 | 0.05818199 | sknsh |
| 20211212_75659_621411_391::fsp_sknsh_0:mG116C | mG116C | 2.04181255 | 0.08072797 | sknsh |
| 20211212_75659_621411_391::fsp_sknsh_0:mG116T | mG116T | 1.997090658 | 0.0868108 | sknsh |
| 20211212_75659_621411_391::fsp_sknsh_0:mG119A | mG119A | 3.604082489 | 0.05831575 | sknsh |
| 20211212_75659_621411_391::fsp_sknsh_0:mG119C | mG119C | 3.401173703 | 0.05649928 | sknsh |
| 20211212_75659_621411_391::fsp_sknsh_0:mG119T | mG119T | 2.179935457 | 0.06613606 | sknsh |
| 20211212_75659_621411_391::fsp_sknsh_0:mG122A | mG122A | 3.755551354 | 0.04845467 | sknsh |
| 20211212_75659_621411_391::fsp_sknsh_0:mG122C | mG122C | 4.104707309 | 0.06127901 | sknsh |
| 20211212_75659_621411_391::fsp_sknsh_0:mG122T | mG122T | 3.530913388 | 0.05776979 | sknsh |
| 20211212_75659_621411_391::fsp_sknsh_0:mG128A | mG128A | 5.349030223 | 0.05308978 | sknsh |
| 20211212_75659_621411_391::fsp_sknsh_0:mG128C | mG128C | 5.337976419 | 0.05205717 | sknsh |
| 20211212_75659_621411_391::fsp_sknsh_0:mG128T | mG128T | 5.47233221 | 0.04722058 | sknsh |
| 20211212_75659_621411_391::fsp_sknsh_0:mG131A | mG131A | 5.275536526 | 0.04791568 | sknsh |
| 20211212_75659_621411_391::fsp_sknsh_0:mG131C | mG131C | 5.312695557 | 0.04799822 | sknsh |
| 20211212_75659_621411_391::fsp_sknsh_0:mG131T | mG131T | 5.210376658 | 0.04570911 | sknsh |
| 20211212_75659_621411_391::fsp_sknsh_0:mG132A | mG132A | 4.810793904 | 0.04919704 | sknsh |
| 20211212_75659_621411_391::fsp_sknsh_0:mG132C | mG132C | 6.256497277 | 0.04445606 | sknsh |
| 20211212_75659_621411_391::fsp_sknsh_0:mG132T | mG132T | 5.17478714 | 0.04562488 | sknsh |
| 20211212_75659_621411_391::fsp_sknsh_0:mG135A | mG135A | 6.793300143 | 0.04786703 | sknsh |
| 20211212_75659_621411_391::fsp_sknsh_0:mG135C | mG135C | 6.934734332 | 0.05189824 | sknsh |
| 20211212_75659_621411_391::fsp_sknsh_0:mG135T | mG135T | 4.915285561 | 0.04565065 | sknsh |
| 20211212_75659_621411_391::fsp_sknsh_0:mG137A | mG137A | 4.702991864 | 0.04958257 | sknsh |
| 20211212_75659_621411_391::fsp_sknsh_0:mG137C | mG137C | 4.700844166 | 0.05060026 | sknsh |
| 20211212_75659_621411_391::fsp_sknsh_0:mG137T | mG137T | 4.702409679 | 0.04810001 | sknsh |
| 20211212_75659_621411_391::fsp_sknsh_0:mG142A | mG142A | 4.731742905 | 0.05450011 | sknsh |
| 20211212_75659_621411_391::fsp_sknsh_0:mG142C | mG142C | 4.823113503 | 0.04927791 | sknsh |
| 20211212_75659_621411_391::fsp_sknsh_0:mG142T | mG142T | 4.792051791 | 0.0523595 | sknsh |
| 20211212_75659_621411_391::fsp_sknsh_0:mG143A | mG143A | 4.552309467 | 0.0542996 | sknsh |
| 20211212_75659_621411_391::fsp_sknsh_0:mG143C | mG143C | 4.836679825 | 0.05741645 | sknsh |
| 20211212_75659_621411_391::fsp_sknsh_0:mG143T | mG143T | 4.900753924 | 0.04952038 | sknsh |
| 20211212_75659_621411_391::fsp_sknsh_0:mG151A | mG151A | 4.681607159 | 0.05797431 | sknsh |
| 20211212_75659_621411_391::fsp_sknsh_0:mG151C | mG151C | 5.15514106 | 0.05578499 | sknsh |
| 20211212_75659_621411_391::fsp_sknsh_0:mG151T | mG151T | 4.972115897 | 0.05336808 | sknsh |
| 20211212_75659_621411_391::fsp_sknsh_0:mG152A | mG152A | 4.937776079 | 0.05419851 | sknsh |
| 20211212_75659_621411_391::fsp_sknsh_0:mG152C | mG152C | 5.256123307 | 0.05549412 | sknsh |
| 20211212_75659_621411_391::fsp_sknsh_0:mG152T | mG152T | 5.240689636 | 0.075879 | sknsh |
| 20211212_75659_621411_391::fsp_sknsh_0:mG159A | mG159A | 4.819500755 | 0.0529595 | sknsh |
| 20211212_75659_621411_391::fsp_sknsh_0:mG159C | mG159C | 5.041784656 | 0.12810813 | sknsh |
| 20211212_75659_621411_391::fsp_sknsh_0:mG159T | mG159T | 4.793130254 | 0.05830746 | sknsh |
| 20211212_75659_621411_391::fsp_sknsh_0:mG161A | mG161A | 4.984208227 | 0.0462394 | sknsh |
| 20211212_75659_621411_391::fsp_sknsh_0:mG161C | mG161C | 4.842721346 | 0.05432754 | sknsh |
| 20211212_75659_621411_391::fsp_sknsh_0:mG161T | mG161T | 4.810108077 | 0.0502712 | sknsh |
| 20211212_75659_621411_391::fsp_sknsh_0:mG166A | mG166A | 4.729367596 | 0.04783738 | sknsh |
| 20211212_75659_621411_391::fsp_sknsh_0:mG166C | mG166C | 4.755695586 | 0.05826415 | sknsh |
| 20211212_75659_621411_391::fsp_sknsh_0:mG166T | mG166T | 4.621128103 | 0.05433322 | sknsh |
| 20211212_75659_621411_391::fsp_sknsh_0:mG169A | mG169A | 4.780341675 | 0.05410358 | sknsh |
| 20211212_75659_621411_391::fsp_sknsh_0:mG169C | mG169C | 4.745930155 | 0.04922569 | sknsh |
| 20211212_75659_621411_391::fsp_sknsh_0:mG169T | mG169T | 4.641364618 | 0.05548388 | sknsh |
| 20211212_75659_621411_391::fsp_sknsh_0:mG16A | mG16A | 4.55107966 | 0.04700523 | sknsh |
| 20211212_75659_621411_391::fsp_sknsh_0:mG16C | mG16C | 4.556031599 | 0.05147461 | sknsh |
| 20211212_75659_621411_391::fsp_sknsh_0:mG16T | mG16T | 4.726791038 | 0.04992858 | sknsh |
| 20211212_75659_621411_391::fsp_sknsh_0:mG170A | mG170A | 4.84766021 | 0.05109021 | sknsh |
| 20211212_75659_621411_391::fsp_sknsh_0:mG170C | mG170C | 4.925932557 | 0.0521661 | sknsh |
| 20211212_75659_621411_391::fsp_sknsh_0:mG170T | mG170T | 4.843299096 | 0.05266348 | sknsh |
| 20211212_75659_621411_391::fsp_sknsh_0:mG172A | mG172A | 4.810505695 | 0.04956228 | sknsh |
| 20211212_75659_621411_391::fsp_sknsh_0:mG172C | mG172C | 4.918266952 | 0.05351953 | sknsh |
| 20211212_75659_621411_391::fsp_sknsh_0:mG172T | mG172T | 4.917805696 | 0.05088618 | sknsh |
| 20211212_75659_621411_391::fsp_sknsh_0:mG176A | mG176A | 4.928370207 | 0.05434144 | sknsh |
| 20211212_75659_621411_391::fsp_sknsh_0:mG176C | mG176C | 5.085963875 | 0.04964232 | sknsh |
| 20211212_75659_621411_391::fsp_sknsh_0:mG176T | mG176T | 4.990075368 | 0.06351763 | sknsh |
| 20211212_75659_621411_391::fsp_sknsh_0:mG183A | mG183A | 4.726757186 | 0.05509722 | sknsh |
| 20211212_75659_621411_391::fsp_sknsh_0:mG183C | mG183C | 4.947255646 | 0.05364475 | sknsh |
| 20211212_75659_621411_391::fsp_sknsh_0:mG183T | mG183T | 4.928312961 | 0.05038882 | sknsh |
| 20211212_75659_621411_391::fsp_sknsh_0:mG184A | mG184A | 4.889590999 | 0.04680632 | sknsh |
| 20211212_75659_621411_391::fsp_sknsh_0:mG184C | mG184C | 5.238957315 | 0.04844108 | sknsh |
| 20211212_75659_621411_391::fsp_sknsh_0:mG184T | mG184T | 4.938471935 | 0.05318188 | sknsh |
| 20211212_75659_621411_391::fsp_sknsh_0:mG187A | mG187A | 4.800378722 | 0.05410019 | sknsh |
| 20211212_75659_621411_391::fsp_sknsh_0:mG187C | mG187C | 4.781395918 | 0.05361523 | sknsh |
| 20211212_75659_621411_391::fsp_sknsh_0:mG187T | mG187T | 4.922141401 | 0.04991082 | sknsh |
| 20211212_75659_621411_391::fsp_sknsh_0:mG18A | mG18A | 4.70714973 | 0.05977398 | sknsh |
| 20211212_75659_621411_391::fsp_sknsh_0:mG18C | mG18C | 4.62628932 | 0.0590389 | sknsh |
| 20211212_75659_621411_391::fsp_sknsh_0:mG18T | mG18T | 4.6753102 | 0.0554303 | sknsh |
| 20211212_75659_621411_391::fsp_sknsh_0:mG19A | mG19A | 4.706050602 | 0.04909407 | sknsh |
| 20211212_75659_621411_391::fsp_sknsh_0:mG19C | mG19C | 6.181070603 | 0.05056552 | sknsh |
| 20211212_75659_621411_391::fsp_sknsh_0:mG19T | mG19T | 5.10408505 | 0.05185313 | sknsh |
| 20211212_75659_621411_391::fsp_sknsh_0:mG21A | mG21A | 5.114379833 | 0.04924068 | sknsh |
| 20211212_75659_621411_391::fsp_sknsh_0:mG21C | mG21C | 5.414207003 | 0.05251248 | sknsh |
| 20211212_75659_621411_391::fsp_sknsh_0:mG21T | mG21T | 5.063428018 | 0.05283389 | sknsh |
| 20211212_75659_621411_391::fsp_sknsh_0:mG22A | mG22A | 4.662891733 | 0.05512232 | sknsh |
| 20211212_75659_621411_391::fsp_sknsh_0:mG22C | mG22C | 4.806389004 | 0.05565593 | sknsh |
| 20211212_75659_621411_391::fsp_sknsh_0:mG22T | mG22T | 4.988495713 | 0.04671515 | sknsh |
| 20211212_75659_621411_391::fsp_sknsh_0:mG30A | mG30A | 4.857706745 | 0.05662812 | sknsh |
| 20211212_75659_621411_391::fsp_sknsh_0:mG30C | mG30C | 4.741510592 | 0.05115343 | sknsh |
| 20211212_75659_621411_391::fsp_sknsh_0:mG30T | mG30T | 4.820441723 | 0.05093231 | sknsh |
| 20211212_75659_621411_391::fsp_sknsh_0:mG40A | mG40A | 5.320080197 | 0.05258142 | sknsh |
| 20211212_75659_621411_391::fsp_sknsh_0:mG40C | mG40C | 5.059708552 | 0.04962961 | sknsh |
| 20211212_75659_621411_391::fsp_sknsh_0:mG40T | mG40T | 5.101222632 | 0.05245363 | sknsh |
| 20211212_75659_621411_391::fsp_sknsh_0:mG43A | mG43A | 5.075990883 | 0.04749958 | sknsh |
| 20211212_75659_621411_391::fsp_sknsh_0:mG43C | mG43C | 5.294228242 | 0.04791534 | sknsh |
| 20211212_75659_621411_391::fsp_sknsh_0:mG43T | mG43T | 4.984317384 | 0.05297361 | sknsh |
| 20211212_75659_621411_391::fsp_sknsh_0:mG52A | mG52A | 5.235529738 | 0.05604024 | sknsh |
| 20211212_75659_621411_391::fsp_sknsh_0:mG52C | mG52C | 5.181440769 | 0.04920512 | sknsh |
| 20211212_75659_621411_391::fsp_sknsh_0:mG52T | mG52T | 5.350539256 | 0.04385856 | sknsh |
| 20211212_75659_621411_391::fsp_sknsh_0:mG5A | mG5A | 4.767338538 | 0.04933727 | sknsh |
| 20211212_75659_621411_391::fsp_sknsh_0:mG5C | mG5C | 4.749904317 | 0.05585108 | sknsh |
| 20211212_75659_621411_391::fsp_sknsh_0:mG5T | mG5T | 4.715948838 | 0.04962951 | sknsh |
| 20211212_75659_621411_391::fsp_sknsh_0:mG60A | mG60A | 5.146003067 | 0.05510669 | sknsh |
| 20211212_75659_621411_391::fsp_sknsh_0:mG60C | mG60C | 5.565229662 | 0.05044989 | sknsh |
| 20211212_75659_621411_391::fsp_sknsh_0:mG60T | mG60T | 5.293390513 | 0.05108689 | sknsh |
| 20211212_75659_621411_391::fsp_sknsh_0:mG61A | mG61A | 4.684711346 | 0.04910585 | sknsh |
| 20211212_75659_621411_391::fsp_sknsh_0:mG61C | mG61C | 5.328867958 | 0.05199375 | sknsh |
| 20211212_75659_621411_391::fsp_sknsh_0:mG61T | mG61T | 4.571519604 | 0.05897506 | sknsh |
| 20211212_75659_621411_391::fsp_sknsh_0:mG62A | mG62A | 5.002277192 | 0.05491472 | sknsh |
| 20211212_75659_621411_391::fsp_sknsh_0:mG62C | mG62C | 5.068183241 | 0.04849175 | sknsh |
| 20211212_75659_621411_391::fsp_sknsh_0:mG62T | mG62T | 5.114712914 | 0.05135036 | sknsh |
| 20211212_75659_621411_391::fsp_sknsh_0:mG63A | mG63A | 5.393503928 | 0.0467058 | sknsh |
| 20211212_75659_621411_391::fsp_sknsh_0:mG63C | mG63C | 4.924048529 | 0.05035458 | sknsh |
| 20211212_75659_621411_391::fsp_sknsh_0:mG63T | mG63T | 4.894836028 | 0.04846528 | sknsh |
| 20211212_75659_621411_391::fsp_sknsh_0:mG68A | mG68A | 4.03776776 | 0.06580739 | sknsh |
| 20211212_75659_621411_391::fsp_sknsh_0:mG68C | mG68C | 4.272273689 | 0.05203249 | sknsh |
| 20211212_75659_621411_391::fsp_sknsh_0:mG68T | mG68T | 4.782969328 | 0.04917434 | sknsh |
| 20211212_75659_621411_391::fsp_sknsh_0:mG71A | mG71A | 4.026753632 | 0.05238851 | sknsh |
| 20211212_75659_621411_391::fsp_sknsh_0:mG71C | mG71C | 4.166132363 | 0.05395793 | sknsh |
| 20211212_75659_621411_391::fsp_sknsh_0:mG71T | mG71T | 5.304590122 | 0.04692065 | sknsh |
| 20211212_75659_621411_391::fsp_sknsh_0:mG79A | mG79A | 5.045006283 | 0.04950654 | sknsh |
| 20211212_75659_621411_391::fsp_sknsh_0:mG79C | mG79C | 4.71290592 | 0.04989 | sknsh |
| 20211212_75659_621411_391::fsp_sknsh_0:mG79T | mG79T | 5.047364939 | 0.04390122 | sknsh |
| 20211212_75659_621411_391::fsp_sknsh_0:mG80A | mG80A | 3.35466443 | 0.05614685 | sknsh |
| 20211212_75659_621411_391::fsp_sknsh_0:mG80C | mG80C | 4.534882553 | 0.04998885 | sknsh |
| 20211212_75659_621411_391::fsp_sknsh_0:mG80T | mG80T | 4.555748723 | 0.05188712 | sknsh |
| 20211212_75659_621411_391::fsp_sknsh_0:mG82A | mG82A | 4.594537548 | 0.0467725 | sknsh |
| 20211212_75659_621411_391::fsp_sknsh_0:mG82C | mG82C | 4.4500478 | 0.04721538 | sknsh |
| 20211212_75659_621411_391::fsp_sknsh_0:mG82T | mG82T | 4.619578265 | 0.046866 | sknsh |
| 20211212_75659_621411_391::fsp_sknsh_0:mG83A | mG83A | 5.109205871 | 0.05081804 | sknsh |
| 20211212_75659_621411_391::fsp_sknsh_0:mG83C | mG83C | 6.600608236 | 0.04467935 | sknsh |
| 20211212_75659_621411_391::fsp_sknsh_0:mG83T | mG83T | 5.527829359 | 0.04975703 | sknsh |
| 20211212_75659_621411_391::fsp_sknsh_0:mG86A | mG86A | 4.407249074 | 0.05914554 | sknsh |
| 20211212_75659_621411_391::fsp_sknsh_0:mG86C | mG86C | 3.456349156 | 0.05387298 | sknsh |
| 20211212_75659_621411_391::fsp_sknsh_0:mG86T | mG86T | 3.959005054 | 0.05286518 | sknsh |
| 20211212_75659_621411_391::fsp_sknsh_0:mG88A | mG88A | 3.744956037 | 0.06231246 | sknsh |
| 20211212_75659_621411_391::fsp_sknsh_0:mG88C | mG88C | 3.521618274 | 0.05211657 | sknsh |
| 20211212_75659_621411_391::fsp_sknsh_0:mG88T | mG88T | 3.97384603 | 0.05093901 | sknsh |
| 20211212_75659_621411_391::fsp_sknsh_0:mG93A | mG93A | 4.951711727 | 0.04705593 | sknsh |
| 20211212_75659_621411_391::fsp_sknsh_0:mG93C | mG93C | 4.846468178 | 0.05270426 | sknsh |
| 20211212_75659_621411_391::fsp_sknsh_0:mG93T | mG93T | 4.625416691 | 0.04919134 | sknsh |
| 20211212_75659_621411_391::fsp_sknsh_0:mG95A | mG95A | 4.545346585 | 0.0501507 | sknsh |
| 20211212_75659_621411_391::fsp_sknsh_0:mG95C | mG95C | 6.608760338 | 0.04999111 | sknsh |
| 20211212_75659_621411_391::fsp_sknsh_0:mG95T | mG95T | 4.912225589 | 0.05088393 | sknsh |
| 20211212_75659_621411_391::fsp_sknsh_0:mG96A | mG96A | 3.891999758 | 0.0527492 | sknsh |
| 20211212_75659_621411_391::fsp_sknsh_0:mG96C | mG96C | 5.149713114 | 0.05176421 | sknsh |
| 20211212_75659_621411_391::fsp_sknsh_0:mG96T | mG96T | 5.039285475 | 0.04936177 | sknsh |
| 20211212_75659_621411_391::fsp_sknsh_0:mT100A | mT100A | 2.992468192 | 0.06800356 | sknsh |
| 20211212_75659_621411_391::fsp_sknsh_0:mT100C | mT100C | 2.518216692 | 0.04712008 | sknsh |
| 20211212_75659_621411_391::fsp_sknsh_0:mT100G | mT100G | 3.357219949 | 0.05870014 | sknsh |
| 20211212_75659_621411_391::fsp_sknsh_0:mT101A | mT101A | 2.361565048 | 0.05185 | sknsh |
| 20211212_75659_621411_391::fsp_sknsh_0:mT101C | mT101C | 2.908385715 | 0.04454346 | sknsh |
| 20211212_75659_621411_391::fsp_sknsh_0:mT101G | mT101G | 3.307245806 | 0.05554658 | sknsh |
| 20211212_75659_621411_391::fsp_sknsh_0:mT104A | mT104A | 4.963253698 | 0.05833077 | sknsh |
| 20211212_75659_621411_391::fsp_sknsh_0:mT104C | mT104C | 4.58486248 | 0.05142229 | sknsh |
| 20211212_75659_621411_391::fsp_sknsh_0:mT104G | mT104G | 6.248263933 | 0.04210731 | sknsh |
| 20211212_75659_621411_391::fsp_sknsh_0:mT105A | mT105A | 3.328381662 | 0.05717986 | sknsh |
| 20211212_75659_621411_391::fsp_sknsh_0:mT105C | mT105C | 3.155351458 | 0.05603805 | sknsh |
| 20211212_75659_621411_391::fsp_sknsh_0:mT105G | mT105G | 4.435345918 | 0.04603043 | sknsh |
| 20211212_75659_621411_391::fsp_sknsh_0:mT11A | mT11A | 5.297500989 | 0.05307229 | sknsh |
| 20211212_75659_621411_391::fsp_sknsh_0:mT11C | mT11C | 5.313547664 | 0.04974874 | sknsh |
| 20211212_75659_621411_391::fsp_sknsh_0:mT11G | mT11G | 4.923901674 | 0.04755085 | sknsh |
| 20211212_75659_621411_391::fsp_sknsh_0:mT123A | mT123A | 4.873903827 | 0.0519414 | sknsh |
| 20211212_75659_621411_391::fsp_sknsh_0:mT123C | mT123C | 4.836774797 | 0.04935688 | sknsh |
| 20211212_75659_621411_391::fsp_sknsh_0:mT123G | mT123G | 4.976347861 | 0.05479185 | sknsh |
| 20211212_75659_621411_391::fsp_sknsh_0:mT125A | mT125A | 6.84471489 | 0.04506104 | sknsh |
| 20211212_75659_621411_391::fsp_sknsh_0:mT125C | mT125C | 4.991346311 | 0.05176631 | sknsh |
| 20211212_75659_621411_391::fsp_sknsh_0:mT125G | mT125G | 4.923420926 | 0.05660487 | sknsh |
| 20211212_75659_621411_391::fsp_sknsh_0:mT126A | mT126A | 5.326609421 | 0.05063599 | sknsh |
| 20211212_75659_621411_391::fsp_sknsh_0:mT126C | mT126C | 5.680274159 | 0.05061319 | sknsh |
| 20211212_75659_621411_391::fsp_sknsh_0:mT126G | mT126G | 5.633952678 | 0.04750331 | sknsh |
| 20211212_75659_621411_391::fsp_sknsh_0:mT134A | mT134A | 5.382327634 | 0.04710687 | sknsh |
| 20211212_75659_621411_391::fsp_sknsh_0:mT134C | mT134C | 5.955193816 | 0.04555476 | sknsh |
| 20211212_75659_621411_391::fsp_sknsh_0:mT134G | mT134G | 5.874031862 | 0.04963664 | sknsh |
| 20211212_75659_621411_391::fsp_sknsh_0:mT145A | mT145A | 4.77348597 | 0.04824604 | sknsh |
| 20211212_75659_621411_391::fsp_sknsh_0:mT145C | mT145C | 5.094190194 | 0.05123681 | sknsh |
| 20211212_75659_621411_391::fsp_sknsh_0:mT145G | mT145G | 5.20530649 | 0.04946747 | sknsh |
| 20211212_75659_621411_391::fsp_sknsh_0:mT146A | mT146A | 5.652135131 | 0.0473783 | sknsh |
| 20211212_75659_621411_391::fsp_sknsh_0:mT146C | mT146C | 5.266584842 | 0.05098239 | sknsh |
| 20211212_75659_621411_391::fsp_sknsh_0:mT146G | mT146G | 5.849585321 | 0.04722303 | sknsh |
| 20211212_75659_621411_391::fsp_sknsh_0:mT147A | mT147A | 5.207907289 | 0.05273664 | sknsh |
| 20211212_75659_621411_391::fsp_sknsh_0:mT147C | mT147C | 4.977841463 | 0.05009687 | sknsh |
| 20211212_75659_621411_391::fsp_sknsh_0:mT147G | mT147G | 5.037228402 | 0.04873902 | sknsh |
| 20211212_75659_621411_391::fsp_sknsh_0:mT14A | mT14A | 5.01157588 | 0.0503767 | sknsh |
| 20211212_75659_621411_391::fsp_sknsh_0:mT14C | mT14C | 5.129302076 | 0.05768623 | sknsh |
| 20211212_75659_621411_391::fsp_sknsh_0:mT14G | mT14G | 5.059637016 | 0.04933101 | sknsh |
| 20211212_75659_621411_391::fsp_sknsh_0:mT155A | mT155A | 4.905147756 | 0.05436637 | sknsh |
| 20211212_75659_621411_391::fsp_sknsh_0:mT155C | mT155C | 5.277394161 | 0.04892737 | sknsh |
| 20211212_75659_621411_391::fsp_sknsh_0:mT155G | mT155G | 5.370780306 | 0.05142991 | sknsh |
| 20211212_75659_621411_391::fsp_sknsh_0:mT156A | mT156A | 5.202138143 | 0.08073295 | sknsh |
| 20211212_75659_621411_391::fsp_sknsh_0:mT156C | mT156C | 5.168631306 | 0.04486834 | sknsh |
| 20211212_75659_621411_391::fsp_sknsh_0:mT156G | mT156G | 5.074798627 | 0.05066782 | sknsh |
| 20211212_75659_621411_391::fsp_sknsh_0:mT157A | mT157A | 5.052399644 | 0.04867166 | sknsh |
| 20211212_75659_621411_391::fsp_sknsh_0:mT157C | mT157C | 5.217539469 | 0.05022587 | sknsh |
| 20211212_75659_621411_391::fsp_sknsh_0:mT157G | mT157G | 5.145074946 | 0.04580188 | sknsh |
| 20211212_75659_621411_391::fsp_sknsh_0:mT162A | mT162A | 5.01765024 | 0.05494135 | sknsh |
| 20211212_75659_621411_391::fsp_sknsh_0:mT162C | mT162C | 5.24378932 | 0.05175626 | sknsh |
| 20211212_75659_621411_391::fsp_sknsh_0:mT162G | mT162G | 5.07246048 | 0.05293961 | sknsh |
| 20211212_75659_621411_391::fsp_sknsh_0:mT165A | mT165A | 4.935735522 | 0.04755313 | sknsh |
| 20211212_75659_621411_391::fsp_sknsh_0:mT165C | mT165C | 5.069031719 | 0.05418896 | sknsh |
| 20211212_75659_621411_391::fsp_sknsh_0:mT165G | mT165G | 4.98278583 | 0.050616 | sknsh |
| 20211212_75659_621411_391::fsp_sknsh_0:mT173A | mT173A | 4.904738514 | 0.05558712 | sknsh |
| 20211212_75659_621411_391::fsp_sknsh_0:mT173C | mT173C | 5.0413252 | 0.04933589 | sknsh |
| 20211212_75659_621411_391::fsp_sknsh_0:mT173G | mT173G | 4.990472225 | 0.0494336 | sknsh |
| 20211212_75659_621411_391::fsp_sknsh_0:mT174A | mT174A | 4.85539324 | 0.04995469 | sknsh |
| 20211212_75659_621411_391::fsp_sknsh_0:mT174C | mT174C | 5.01454466 | 0.04960424 | sknsh |
| 20211212_75659_621411_391::fsp_sknsh_0:mT174G | mT174G | 5.017401741 | 0.04896286 | sknsh |
| 20211212_75659_621411_391::fsp_sknsh_0:mT175A | mT175A | 4.984941997 | 0.04941188 | sknsh |
| 20211212_75659_621411_391::fsp_sknsh_0:mT175C | mT175C | 5.093796934 | 0.05677646 | sknsh |
| 20211212_75659_621411_391::fsp_sknsh_0:mT175G | mT175G | 4.940139502 | 0.04979779 | sknsh |
| 20211212_75659_621411_391::fsp_sknsh_0:mT177A | mT177A | 4.964890384 | 0.051322 | sknsh |
| 20211212_75659_621411_391::fsp_sknsh_0:mT177C | mT177C | 5.103935708 | 0.05187509 | sknsh |
| 20211212_75659_621411_391::fsp_sknsh_0:mT177G | mT177G | 4.688221144 | 0.10354807 | sknsh |
| 20211212_75659_621411_391::fsp_sknsh_0:mT178A | mT178A | 5.001967606 | 0.05574256 | sknsh |
| 20211212_75659_621411_391::fsp_sknsh_0:mT178C | mT178C | 5.028133126 | 0.05606972 | sknsh |
| 20211212_75659_621411_391::fsp_sknsh_0:mT178G | mT178G | 4.971770514 | 0.05526356 | sknsh |
| 20211212_75659_621411_391::fsp_sknsh_0:mT182A | mT182A | 5.063305589 | 0.0477424 | sknsh |
| 20211212_75659_621411_391::fsp_sknsh_0:mT182C | mT182C | 4.948560767 | 0.04613726 | sknsh |
| 20211212_75659_621411_391::fsp_sknsh_0:mT182G | mT182G | 5.088532826 | 0.05990757 | sknsh |
| 20211212_75659_621411_391::fsp_sknsh_0:mT185A | mT185A | 5.074667546 | 0.05284578 | sknsh |
| 20211212_75659_621411_391::fsp_sknsh_0:mT185C | mT185C | 5.281174164 | 0.04661161 | sknsh |
| 20211212_75659_621411_391::fsp_sknsh_0:mT185G | mT185G | 5.100873369 | 0.05380858 | sknsh |
| 20211212_75659_621411_391::fsp_sknsh_0:mT189A | mT189A | 4.946093148 | 0.05046009 | sknsh |
| 20211212_75659_621411_391::fsp_sknsh_0:mT189C | mT189C | 5.018040251 | 0.05036124 | sknsh |
| 20211212_75659_621411_391::fsp_sknsh_0:mT189G | mT189G | 5.007116839 | 0.05208253 | sknsh |
| 20211212_75659_621411_391::fsp_sknsh_0:mT190A | mT190A | 4.966479086 | 0.04757965 | sknsh |
| 20211212_75659_621411_391::fsp_sknsh_0:mT190C | mT190C | 5.114341585 | 0.04911223 | sknsh |
| 20211212_75659_621411_391::fsp_sknsh_0:mT190G | mT190G | 4.969708072 | 0.04914812 | sknsh |
| 20211212_75659_621411_391::fsp_sknsh_0:mT196A | mT196A | 5.114292265 | 0.24974348 | sknsh |
| 20211212_75659_621411_391::fsp_sknsh_0:mT196C | mT196C | 5.490581569 | 0.30592643 | sknsh |
| 20211212_75659_621411_391::fsp_sknsh_0:mT196G | mT196G | 5.275639431 | 0.32161002 | sknsh |
| 20211212_75659_621411_391::fsp_sknsh_0:mT1A | mT1A | 5.04767645 | 0.35243175 | sknsh |
| 20211212_75659_621411_391::fsp_sknsh_0:mT1C | mT1C | 4.391094247 | 0.26858528 | sknsh |
| 20211212_75659_621411_391::fsp_sknsh_0:mT1G | mT1G | 4.765197696 | 0.05085989 | sknsh |
| 20211212_75659_621411_391::fsp_sknsh_0:mT200A | mT200A | 5.019698447 | 0.17916047 | sknsh |
| 20211212_75659_621411_391::fsp_sknsh_0:mT200C | mT200C | 5.02363295 | 0.46303681 | sknsh |
| 20211212_75659_621411_391::fsp_sknsh_0:mT200G | mT200G | 4.965556494 | 0.25375962 | sknsh |
| 20211212_75659_621411_391::fsp_sknsh_0:mT25A | mT25A | 4.656375945 | 0.05568583 | sknsh |
| 20211212_75659_621411_391::fsp_sknsh_0:mT25C | mT25C | 4.577358552 | 0.05417409 | sknsh |
| 20211212_75659_621411_391::fsp_sknsh_0:mT25G | mT25G | 5.147305797 | 0.05254208 | sknsh |
| 20211212_75659_621411_391::fsp_sknsh_0:mT27A | mT27A | 4.888250334 | 0.04456588 | sknsh |
| 20211212_75659_621411_391::fsp_sknsh_0:mT27C | mT27C | 5.033007972 | 0.04995417 | sknsh |
| 20211212_75659_621411_391::fsp_sknsh_0:mT27G | mT27G | 4.811653691 | 0.04582016 | sknsh |
| 20211212_75659_621411_391::fsp_sknsh_0:mT33A | mT33A | 5.399827759 | 0.04915392 | sknsh |
| 20211212_75659_621411_391::fsp_sknsh_0:mT33C | mT33C | 4.942874326 | 0.04820795 | sknsh |
| 20211212_75659_621411_391::fsp_sknsh_0:mT33G | mT33G | 5.055980364 | 0.04851773 | sknsh |
| 20211212_75659_621411_391::fsp_sknsh_0:mT35A | mT35A | 5.171276283 | 0.04777721 | sknsh |
| 20211212_75659_621411_391::fsp_sknsh_0:mT35C | mT35C | 4.908745977 | 0.05202014 | sknsh |
| 20211212_75659_621411_391::fsp_sknsh_0:mT35G | mT35G | 5.022641352 | 0.05119698 | sknsh |
| 20211212_75659_621411_391::fsp_sknsh_0:mT36A | mT36A | 4.976266357 | 0.0498108 | sknsh |
| 20211212_75659_621411_391::fsp_sknsh_0:mT36C | mT36C | 5.037705237 | 0.05433823 | sknsh |
| 20211212_75659_621411_391::fsp_sknsh_0:mT36G | mT36G | 5.035176251 | 0.05192615 | sknsh |
| 20211212_75659_621411_391::fsp_sknsh_0:mT3A | mT3A | 5.571211293 | 0.66445723 | sknsh |
| 20211212_75659_621411_391::fsp_sknsh_0:mT3C | mT3C | 5.089300178 | 0.18277205 | sknsh |
| 20211212_75659_621411_391::fsp_sknsh_0:mT3G | mT3G | 6.254463281 | 0.43913095 | sknsh |
| 20211212_75659_621411_391::fsp_sknsh_0:mT47A | mT47A | 5.042614739 | 0.04922756 | sknsh |
| 20211212_75659_621411_391::fsp_sknsh_0:mT47C | mT47C | 5.069334356 | 0.04985615 | sknsh |
| 20211212_75659_621411_391::fsp_sknsh_0:mT47G | mT47G | 5.074980136 | 0.04683602 | sknsh |
| 20211212_75659_621411_391::fsp_sknsh_0:mT49A | mT49A | 5.167909574 | 0.05606279 | sknsh |
| 20211212_75659_621411_391::fsp_sknsh_0:mT49C | mT49C | 6.863714528 | 0.04800189 | sknsh |
| 20211212_75659_621411_391::fsp_sknsh_0:mT49G | mT49G | 5.136300809 | 0.05272463 | sknsh |
| 20211212_75659_621411_391::fsp_sknsh_0:mT55A | mT55A | 5.105311029 | 0.04733681 | sknsh |
| 20211212_75659_621411_391::fsp_sknsh_0:mT55C | mT55C | 4.936395995 | 0.04423658 | sknsh |
| 20211212_75659_621411_391::fsp_sknsh_0:mT55G | mT55G | 5.475094199 | 0.04694622 | sknsh |
| 20211212_75659_621411_391::fsp_sknsh_0:mT58A | mT58A | 5.229445865 | 0.04751685 | sknsh |
| 20211212_75659_621411_391::fsp_sknsh_0:mT58C | mT58C | 5.33394932 | 0.0535694 | sknsh |
| 20211212_75659_621411_391::fsp_sknsh_0:mT58G | mT58G | 5.706843534 | 0.04753225 | sknsh |
| 20211212_75659_621411_391::fsp_sknsh_0:mT65A | mT65A | 4.923986794 | 0.05017541 | sknsh |
| 20211212_75659_621411_391::fsp_sknsh_0:mT65C | mT65C | 4.902831239 | 0.0526227 | sknsh |
| 20211212_75659_621411_391::fsp_sknsh_0:mT65G | mT65G | 5.290534918 | 0.05298097 | sknsh |
| 20211212_75659_621411_391::fsp_sknsh_0:mT66A | mT66A | 6.527931429 | 0.0475819 | sknsh |
| 20211212_75659_621411_391::fsp_sknsh_0:mT66C | mT66C | 5.623996232 | 0.05193098 | sknsh |
| 20211212_75659_621411_391::fsp_sknsh_0:mT66G | mT66G | 6.548669926 | 0.04965617 | sknsh |
| 20211212_75659_621411_391::fsp_sknsh_0:mT67A | mT67A | 4.320895791 | 0.05381778 | sknsh |
| 20211212_75659_621411_391::fsp_sknsh_0:mT67C | mT67C | 4.174829274 | 0.05913548 | sknsh |
| 20211212_75659_621411_391::fsp_sknsh_0:mT67G | mT67G | 5.750200439 | 0.04842793 | sknsh |
| 20211212_75659_621411_391::fsp_sknsh_0:mT6A | mT6A | 4.656352239 | 0.42927082 | sknsh |
| 20211212_75659_621411_391::fsp_sknsh_0:mT6C | mT6C | 4.857189235 | 0.04636612 | sknsh |
| 20211212_75659_621411_391::fsp_sknsh_0:mT6G | mT6G | 4.220253 | 0.4727579 | sknsh |
| 20211212_75659_621411_391::fsp_sknsh_0:mT74A | mT74A | 5.40262574 | 0.0589043 | sknsh |
| 20211212_75659_621411_391::fsp_sknsh_0:mT74C | mT74C | 4.73252564 | 0.04689727 | sknsh |
| 20211212_75659_621411_391::fsp_sknsh_0:mT74G | mT74G | 5.462662506 | 0.05208417 | sknsh |
| 20211212_75659_621411_391::fsp_sknsh_0:mT77A | mT77A | 5.089765202 | 0.05457064 | sknsh |
| 20211212_75659_621411_391::fsp_sknsh_0:mT77C | mT77C | 4.837167295 | 0.05501434 | sknsh |
| 20211212_75659_621411_391::fsp_sknsh_0:mT77G | mT77G | 5.522798753 | 0.04724438 | sknsh |
| 20211212_75659_621411_391::fsp_sknsh_0:mT85A | mT85A | 4.569793478 | 0.05404591 | sknsh |
| 20211212_75659_621411_391::fsp_sknsh_0:mT85C | mT85C | 4.173866864 | 0.05362963 | sknsh |
| 20211212_75659_621411_391::fsp_sknsh_0:mT85G | mT85G | 4.825257021 | 0.05233225 | sknsh |
| 20211212_75659_621411_391::fsp_sknsh_0:mT89A | mT89A | 3.639687152 | 0.05429684 | sknsh |
| 20211212_75659_621411_391::fsp_sknsh_0:mT89C | mT89C | 5.77956098 | 0.05129396 | sknsh |
| 20211212_75659_621411_391::fsp_sknsh_0:mT89G | mT89G | 4.061718106 | 0.05243462 | sknsh |
| 20211212_75659_621411_391::fsp_sknsh_0:mT92A | mT92A | 4.79606354 | 0.05237738 | sknsh |
| 20211212_75659_621411_391::fsp_sknsh_0:mT92C | mT92C | 4.349517708 | 0.05122382 | sknsh |
| 20211212_75659_621411_391::fsp_sknsh_0:mT92G | mT92G | 4.988633816 | 0.04835698 | sknsh |
| 20211212_75659_621411_391::fsp_sknsh_0:mT9A | mT9A | 4.260349157 | 0.50534136 | sknsh |
| 20211212_75659_621411_391::fsp_sknsh_0:mT9C | mT9C | 5.159879328 | 0.1951413 | sknsh |
| 20211212_75659_621411_391::fsp_sknsh_0:mT9G | mT9G | 4.901092727 | 0.0494383 | sknsh |

Table Header Descriptions:
ID = oligo ID;
sat_mut = allele ID: m{reference allele}{position}{alternate allele};
log2FoldChange = mean across replicates of the log2(Fold Change) in SKNSH;
IfcSE = standar error of the log2(Fold Change) across replicates;
celltype = cell type where MPRA was conducted

### CODA MPRA

MPRA library construction: CODA MPRA library was constructed following protocols previously described in Tewhey et al. 2016 13. In brief, oligos were synthesized (Twist Bioscience) as 230 bp sequences containing 200 bp of genomic sequences and 15 bp of adaptor sequence on either end. The oligo library was PCR amplified with primers MPRA_v3_F and MPRA_v3_20I_R to add unique 20 bp barcodes along with arms for Gibson assembly into a backbone vector. The oligonucleotide library was assembled into pMPRAv3: Aluc: Axbal (Addgene plasmid #109035) and expanded by electroporation into E. coli. Seven of the ten expanded cultures were purified using Qiagen Plasmid Plus Midi Kit to reach 200-300 colony-forming units (barcodes) per oligonucleotide. The expanded plasmid library was sequenced on an Illumina NovaSeq using 2×150 bp chemistry to acquire oligo-barcode pairings. The library underwent AsiSI restriction digestion, and GFP with a minimal promoter amplified from pMPRAv3: minP-GFP (Addgene plasmid #109036) using primers MPRA_v3_GFP_Fusion_F and MPRA_v3_GFP_Fusion_R was inserted by Gibson assembly resulting in the 200 bp oligo sequence positioned directly upstream of the promoter and the 20 bp barcode falling in 3′ UTR of GFP. Finally, the library was expanded within E. coli and purified using the Qiagen Plasmid Plus Giga Kit.

MPRA library transfection into cells: Two hundred million cells were transfected using the Neon Transfection System 100ul Kit with 5ug or 10ug of the MPRA library per ten million cells. Cells were harvested 24 hours post transfection, rinsed with PBS and collected by centrifugation. After adding RLT buffer (Rneasy Maxi kit), dithiothreitol and homogenization, cell pellets were frozen at −80° C. until further processing. For each cell type, 3 biological replicates performed on different days.

RNA isolation and MPRA RNA library generation: RNA was extracted from frozen cell homogenates using the Qiagen RNeasy Maxi kit. Following DNase treatment, a mixture of 3 GFP-specific biotinylated primers were used to capture GFP transcripts using Sera Mag Beads (Fisher Scientific). After a second round of DNase treatment, cDNA was synthesized using SuperScript III (Life Technologies) and GFP mRNA abundance was quantified by qPCR to determine the cycle at which linear amplification begins for each replicate. Replicates were diluted to approximately the same concentration based on the qPCR results, and first round PCR (8 or 9 cycles) with primers MPRA_Illumina_GFP_F_v2 and Ilmn P5_1stPCR_v2 were used to amplify barcodes associated with GFP mRNA sequences for each replicate. A second round of PCR (6 cycles) was used to add Illumina sequencing adaptors to the replicates. The resulting Illumina indexed MPRA barcode libraries were sequenced on an Illumina NovaSeq using 1×20 bp chemistry.

### CRE Prioritization for In Vivo Validation

Enformer analysis of epigenetic signatures: To simulate epigenetic and gene expression signatures i n silico we collected the nucleotide sequence from chr11:3, 101, 137-3,493,091 of the mouse reference genome (mm 10). The expected insertion sequence using an H11 targeting vector with a lacZ: P2A: GFP open reading frame was added. As a control, the expected CRE insertion site was simulated as a 200 nucleotide sequence of N. We simulated all possible CRE insertions corresponding to our cell type-specific MPRA by replacing the oligo-N sequence with 200-mers from our library. We inferred epigenetic signatures for all of these sequences using Enformer by modifying the notebook provided by this link (colab.research.google.com/github/deepmind/deepmind_research/blob/master/enformer/enformer-usage.ipynb). To estimate CRE induced transcriptional activation in various tissues we collected 128 nucleotide resolution DHS, H3K27ac, ATAC, and CAGE datasets overlapping the expected insertion (35 bins). To calculate an aggregate effect for each tissue, we calculated the max signal for each feature over the insertion, followed by a feature-specific Yeo-Johnson power transformation. Normalized features were then selected based on tissue correspondence (Supplementary Table 8 of Gosai et al. “Machine-guided design of synthetic cell type-specific cis-regulatory elements” BioRxiv doi: doi.org/10.1101/2023.08.08.552077 (2023)) and averaged to estimate CRE activity in 10 different tissues. Applicant calculated MinGap values for spleen, liver, and brain using these 10 measurements for each CRE.

Manual sequence prioritization: Sequences were prioritized based on review of empirical MPRA measurements, contribution scores, motif matches, sequence content, and predicted epigenetic signatures. Applicant looked for sequences that displayed a high separation between the MPRA measures of the target and the off-target cell types. Applicant also looked to capture variations of combinations of motif matches, and we used the contribution scores to visually examine the motif matches and other potentially important sequence content. Finally, Applicant selected sequences with at least moderate tissue specificity in predicted epigenetic signatures.

### Transgenics

Transient zebrafish synthetic enhancer assay. To build the synthetic CRE eGFP reporter, double-stranded oligonucleotides corresponding to synthetic CREs (200 bp) were synthesized by IDT (GeneBlock). Synthetic CREs were amplified by PCR with primers that included homology to the plasmid vector E1b-GFP-Tol2 (Addgene plasmid #37845) 85 and were cloned upstream of the minimal promoter (E1b) to generate the synthetic enhancer eGFP plasmid reporter (pTol2-synthetic CRE-E1b-eGFP-Tol2) using HiFi DNA Assembly following manufacturer's instructions (New England Biolabs). Applicant also created ‘empty vectors’ which were identical to CODA CRE vectors except for the lack of a 200-bp insert. Reporter plasmid sequences were verified by Sanger sequencing. To transiently express the synthetic CRE reporter in zebrafish, plasmids were co-injected with tol2 transposase mRNA into 1-cell stage zebrafish embryos following established methods 100. Injected embryos were imaged at the indicated days (2 or 4 days-post-fertilization) either by dissecting (Olympus) or confocal fluorescence (Leica SP 8) microscope. All zebrafish procedures were approved by the Yale University Institutional Animal Care and Use Committee (IACUC) (Protocol Number 2022-20274).

Mouse transgenic reporter assay. An H11 targeting vector with an lacZ: P2A: GFP open reading frame was linearized using PCR containing 2 ng of template, 1 ul of KOD Xtreme Hot Start DNA Polymerase (Sigma 71975), 25 ul of Xtreme buffer, and 0.5 μM forward and reverse primers (H11_bxb_lacZ: GFP_lin_F, pGL_minP_GFP_R; Supplementary Table 9 of Gosai et al. “Machine-guided design of synthetic cell type-specific cis-regulatory elements” BioRxiv doi: doi.org/10.1101/2023.08.08.552077 (2023) cycled with the following conditions: 94° C. for 2 min, 20 cycles of 98° C. for 10 s, 56° C. for 30 s, and 68° C. for 13 min, and then 68° C. for 5 min. Amplified fragments were treated with 0.5 uL of DpnI (NEB, R0176S) for 30 min at 37° C., purified using 1× volume of AMPure XP (Beckman Coulter, A63881) and eluted with water. Double-stranded oligonucleotides corresponding to synthetic enhancers with gibson arms were synthesized by IDT (GeneBlock) and assembled into targeting vector using 5 μl of NEBuilder HiFi DNA Assembly Master Mix (NEB, E2621S), 36 ng of linearized vector, and 10 ng of the synthesized fragment in 20 μl total volume for 45 min at 50° C. Transgenic mice were created following the enSERT protocol86. A mixture of 20 ng/μl Cas9 protein (IDT 1074181), 50 ng/μl single guide RNA (sgRNA_H1llacZ; Supplementary Table 9 of Gosai et al. “Machine-guided design of synthetic cell type-specific cis-regulatory elements” BioRxiv doi: doi.org/10.1101/2023.08.08.552077 (2023)), 25 ng/μl donor plasmid, 10 mM Tris, pH 7.5, and 0.1 mM EDTA was injected into pronuclear of FBV zygotes. The whole embryo at E14.5 or isolated brain at 5 weeks postnatal were fixed at 4° C. for 1 hour in PBS supplemented with 2% paraformaldehyde, 0.2% glutaraldehyde, and 0.2% IGEPAL CA-630. After washing with PBS, the embryos were stained at 37° C. overnight in a solution in PBS supplemented with 0.5 mg/ml X-gal (Sigma, B4252), 5 mM potassium hexacyanoferrate (II) trihydrate, 5 mM potassium hexacyanoferrate (III), 2 mM MgCl2, and 0.2% IGEPAL CA-630. The images were taken using Leica M165 for embryos or Leica M125 for brains. All mouse procedures were performed in accordance with the National Institutes of Health Guide for the Care and Use of Laboratory Animals, and were approved by the Institutional Animal Care and Use Committees of The Jackson Laboratory (protocol number 18038).

Histology and immunofluorescence staining. Following LacZ staining, mouse brains were sectioned with a vibratome (Leica VT100s) and free-floating 70 μm-thick sagittal sections were collected in ice-cold PBS. The sections were then rinsed in 1×PBS for 5 minutes and incubated for 30 min in a blocking solution consisting of 0.3% Triton-X, 0.3% mouse on mouse blocking reagent (Vector laboratories, MKB-2213-1), 10% normal goat serum (abcam, ab7481) and 5% BSA in 1×PBS with gentle agitation at room temperature. Immunostaining was then performed with a mixture of primary antibodies in the blocking solution at 4° C. on a shaker overnight. Sections were rinsed in 1×PBS 3 times for 5 minutes each and then incubated with corresponding fluorescence conjugated secondary antibodies for 2 h. After treatment with secondary antibodies, slices were then further rinsed with PBS 3 times, followed by staining for nuclei with DAPI (ThermoFisher Scientific Cat: 62248). Sections were mounted on slides with Prolong Gold antifade reagent (Cell Signalling Technology, #9071). The following primary antibodies were used during the staining procedure: mouse anti-NeuN (abcam ab 104224), chicken anti-GFAP (OriGene Technologies TA309150), rabbit anti-Ibal (abcam ab178846). Secondary antibodies used were Goat anti-mouse Alexa Flour 488 (ThermoFisher Scientific, AB_2534069), Goat anti-chicken Alexa Flour 568 (ThermoFisher Scientific, AB_2534098), Goat anti-rabbit Alexa fluor 568 (abcam, ab175471). All primary and secondary antibodies were used at 1:500 dilutions. Image acquisition Whole-brain sagittal slice mosaic images were acquired with the Thunder Imager (Leica Microsystems) using 10x/NA 0.8 dry lens. Fluorescent imaging was combined with brightfield imaging to visualize LacZ staining. Computational tissue clearing was applied systematically to reduce background noise (Leica acquisition software). After obtaining mosaic scans, higher magnification images of regions of interest (ROI) were acquired on the Stellaris 8 (Leica Microsystems) equipped with a Diode, Ar-gas and He/Ne adjustable wavelength lasers using 40x/NA 1.2 and 63x/NA 1.4 oil objectives for quantification and representative images respectively. Pinhole size was set to 1A.U. and samples were i Illuminated with 405, 488, 561, and 633 nm lasers sequentially. Six-m z-stack images of 2 μm z-step size with 4096×4096-pixel resolution were acquired using HyD detectors with a line average of 3. Fluorescent LacZ staining was visualized with the confocal microscope using the 633 nm laser101. For representative images shown, bright outliers were removed using the default 2-pixel radius and 20 threshold. A gaussian blur was then applied with a sigma radius of 1.

LacZ layer intensity analysis. Acquired mosaic brightfield images underwent auto-thresholding using the Default algorithm in the FIJI software (NIH). Quantification of LacZ signal intensity was achieved using the plot profile tool with ROIs drawn from superficial cortical layers down to the corpus callosum. Depth information for cortical layers was acquired from the Allen Brain atlas. Multiple ROIs were taken in different cortical areas to verify the distribution of the signal. Representative images are ROIs taken from the somatosensory and visual cortices. Cell quantification and overlap analysis To quantify cell populations, using FIJI software, maximum intensity projection of the z-stack of images acquired with a confocal microscope was performed, and background removal was applied with rolling ball radius of 50. The images were then subject to auto-thresholding using the Moments algorithm. SNR was uniform across ROIs and a single thresholding algorithm yielded reproducible results. Cells were then quantified using the Analyze particle function. By varying particle size, accurate quantification of neurons, astrocytes, and microglia was achieved. To calculate the overlap between LacZ expression and the cell-type specific markers, each binarized LacZ image was multiplied with corresponding binarized neuronal, astrocytic and microglia ROIs and the residual signals were quantified using the Analyze particle function. In total, 5 sagittal slices were analyzed per mouse and a total of n=3 mice were used for both controls and LacZ positive brains.

RNA-seq. Three replicates each from transgenic mice of CODA-designed SK-N-SH-specific CRE and empty vector are harvested at 5 weeks postnatal. Liver, spleen and the right half of the brain are soaked into RNA later (Thermo Fisher) overnight at 4° C. and homogenized in QIAzol, followed by a total RNA isolation using RNeasy mini (QIAGEN) with on-column DNase treatment. RNAseq library is generated from 1 μg of total RNA using NEBNext Ultra II RNA Library Prep Kit for Illumina (NEB) and NEBNext Poly (A) mRNA Magnetic Isolation Module

(NEB) following manufacturer's protocol. The libraries are indexed using i7 and i5 primers with the following conditions: 98° C. for 30 s and 10 cycles of (98° C. for 10 s, 65° C. for 75 s), 65° C. for 5 min . . . . Indexed samples were purified using 0.9× volume of AMpure XP, eluted in 20 μL of EB, pooled equimolarly, and sequenced using 2×150 bp chemistry on an Illumina NovaSeq X+ instrument at the Jackson Laboratory. The sequence reads are mapped on a modified mouse genome (GRCm38/mm10) with LacZ-GFP sequence as an additional chromosome using STAR 102 (version 2.5.2b). After removed duplicates using picard MarkDuplicates (MIT, v3.1.1), the mapped reads are counted using featureCount (v2.0.6, options:-p-B-Q 20-T 16-s 2—countReadPairs) DESeq2 (v1.32.0) 103 i s used to normalize the read counts and calculate log₂ fold change, standard error and p-values for Wald test.

### Data Availability

Reference data sets used in this study are linked and annotated in Supplementary Table 1 of Gosai et al. “Machine-guided design of synthetic cell type-specific cis-regulatory elements” BioRxiv doi: doi.org/10.1101/2023.08.08.552077 (2023). Processed MPRA data used to train Malinois is available in Supplementary Table 2 of Gosai et al. “Machine-guided design of synthetic cell type-specific cis-regulatory elements” BioRxiv doi: doi.org/10.1101/2023.08.08.552077 (2023). Processed MPRA data and Malinois predictions for the cell type-specific CRE library designed for this study are available in Supplementary Table 10 of Gosai et al. “Machine-guided design of synthetic cell type-specific cis-regulatory elements” BioRxiv doi: doi.org/10.1101/2023.08.08.552077 (2023). Sequencing reads for RNA-seq are available in NCBI GEO (PRJNA1075667).

### Code Availability

CODA is available at github.com/sjgosai/boda2.

### REFERENCES FOR EXAMPLE 5

1. Wittkopp, P. J. & Kalay, G. Cis-regulatory elements: molecular mechanisms and evolutionary processes underlying divergence. Nat. Rev. Genet. 13, 59-69 (2011).
2. Gasperini, M., Tome, J. M. & Shendure, J. Towards a comprehensive catalogue of validated and target-linked human enhancers. Nat. Rev. Genet. 21, 292-310 (2020).
3. Heinz, S., Romanoski, C. E., Benner, C. & Glass, C. K. The selection and function of cell type-specific enhancers. Nat. Rev. Mol. Cell Biol. 16, 144-154 (2015).
4. de Boer, C. G. & Taipale, J. Hold out the genome: a roadmap to solving the cis-regulatory code. Nature 625, 41-50 (2024).
5. ENCODE Project Consortium et al. Expanded encyclopaedias of DNA elements in the human and mouse genomes. Nature 583, 699-710 (2020).
6. Meuleman, W. et al. Index and biological spectrum of human DNase I hypersensitive sites. Nature 584, 244-251 (2020).
7. Donohue, L. K. H. et al. A cis-regulatory lexicon of DNA motif combinations mediating cell-type-specific gene regulation. Cell Genom 2, (2022).
8. Levo, M. & Segal, E. In pursuit of design principles of regulatory sequences. Nat. Rev. Genet. 15, 453-468 (2014).
9. Avsec, Ž. et al. Base-resolution models of transcription-factor binding reveal soft motif syntax. Nat. Genet. 53, 354-366 (2021).
10. Lambert, S. A. et al. The Human Transcription Factors. Cell 172, 650-665 (2018).
11. Kim, D. S. et al. The dynamic, combinatorial cis-regulatory lexicon of epidermal differentiation. Nat. Genet. 53, 1564-1576 (2021).
12. Shrikumar, A., Greenside, P. & Kundaje, A. Learning Important Features Through Propagating Activation Differences. in Proceedings of the 34th International Conference on Machine Learning (eds. Precup, D. & Teh, Y. W.) vol. 70 3145-3153 (PMLR, 06-11 Aug. 2017).
13. Tewhey, R. et al. Direct Identification of Hundreds of Expression-Modulating Variants using a Multiplexed Reporter Assay. Cell 165, 1519-1529 (2016).
14. Ulirsch, J. C. et al. Systematic Functional Dissection of Common Genetic Variation Affecting Red Blood Cell Traits. Cell 165, 1530-1545 (2016).
15. Ernst, J. et al. Genome-scale high-resolution mapping of activating and repressive nucleotides in regulatory regions. Nat. Biotechnol. 34, 1180-1190 (2016).
16. Melnikov, A. et al. Systematic dissection and optimization of inducible enhancers in human cells using a massively parallel reporter assay. Nat. Biotechnol. 30, 271-277 (2012).
17. Klein, J. C. et al. A systematic evaluation of the design and context dependencies of massively parallel reporter assays. Nat. Methods 17, 1083-1091 (2020).
18. Lawler, A. J. et al. Machine learning sequence prioritization for cell type-specific enhancer design. Elife 11, (2022).
19. Movva, R. et al. Deciphering regulatory DNA sequences and noncoding genetic variants using neural network models of massively parallel reporter assays. PLoS One 14, e0218073 (2019).
20. Vaishnav, E. D. et al. The evolution, evolvability and engineering of gene regulatory DNA. Nature 603, 455-463 (2022).
21. Agarwal, V. et al. Massively parallel characterization of transcriptional regulatory elements i n three diverse human cell types. bioRxiv (2023) doi: 10.1101/2023.03.05.531189
22. Xue, J. R. et al. The functional and evolutionary i mpacts of human-specific deletions in conserved elements. Science 380, eabn2253 (2023).
23. Siraj, L. & Ulirsch, J. Functional dissection of complex and molecular trait variants at single nucleotide resolution. In Preparation (2023).
24. Rosenberg, A. B., Patwardhan, R. P., Shendure, J. & Seelig, G. Learning the sequence determinants of alternative splicing from millions of random sequences. Cell 163, 698-711 (2015).
25. Bogard, N., Linder, J., Rosenberg, A. B. & Seelig, G. A Deep Neural Network for Predicting and Engineering Alternative Polyadenylation. Cell 178, 91-106.e23 (2019).
26. Sample, P. J. et al. Human 5′ UTR design and variant effect prediction from a massively parallel translation assay. Nat. Biotechnol. 37, 803-809 (2019).
27. Kelley, D. R. et al. Sequential regulatory activity prediction across chromosomes with convolutional neural networks. Genome Res. 28, 739-750 (2018).
28. Zhou, J. & Troyanskaya, O. G. Predicting effects of noncoding variants with deep 1 earning-based sequence model. Nat. Methods 12, 931-934 (2015).
29 Quang, D. & Xie, X. DanQ: a hybrid convolutional and recurrent deep neural network for quantifying the function of DNA sequences. Nucleic Acids Res. 44, e107 (2016).
30. Jaganathan, K. et al. Predicting Splicing from Primary Sequence with Deep Learning. Cell 176, 535-548.e24 (2019).
31. de Almeida, B. P., Reiter, F., Pagani, M. & Stark, A. DeepSTARR predicts enhancer activity from DNA sequence and enables the de novo design of synthetic enhancers. Nat. Genet. 54, 613-624 (2022).
32. Penzar, D. et al. LegNet: a best-in-class deep 1 earning model for short DNA regulatory regions. Bioinformatics 39, (2023).
33. Avsec, Ž. et al. Effective gene expression prediction from sequence by integrating 1 ong-range interactions. Nat. Methods 18, 1196-1203 (2021).
34. Sinai, S. & Kelsic, E. D. A primer on model-guided exploration of fitness landscapes for biological sequence design. arXiv [q-bio.QM] (2020).
35. Sinai, S. et al. AdaLead: A simple and robust adaptive greedy search algorithm for sequence design. arXiv [cs.LG] (2020).
36. Linder, J. & Seelig, G. Fast activation maximization for molecular sequence design. BMC Bioinformatics 22, 510 (2021).
37 Zrimec, J. et al. Controlling gene expression with deep generative design of regulatory DNA. Nat. Commun. 13, 5099 (2022).
38. Gupta, A. & Kundaje, A. Targeted optimization of regulatory DNA sequences with neural editing architectures. bioRxiv 714402 (2019) doi: 10.1101/714402.
39. Killoran, N., Lee, L. J., Delong, A., Duvenaud, D. & Frey, B. J. Generating and designing DNA with deep generative models. arXiv [cs.LG] (2017).
40. de Almeida, B. P. et al. Targeted design of synthetic enhancers for selected tissues in the Drosophila embryo. Nature 626, 207-211 (2024).
41. Taskiran, I. I. et al. Cell-type-directed design of synthetic enhancers. Nature 626, 212-220 (2023).
42. Deverman, B. E., Ravina, B. M., Bankiewicz, K. S., Paul, S. M. & Sah, D. W. Y. Gene therapy for neurological disorders: progress and prospects. Nat. Rev. Drug Discov. 17, 767 (2018).
43. Mitchell, M. J. et al. Engineering precision nanoparticles for drug delivery. Nat. Rev. Drug Discov. 20, 101-124 (2020).
44. Tabebordbar, M. et al. Directed evolution of a family of AAV capsid variants enabling potent muscle-directed gene delivery across species. Cell 184, 4919-4938.e22 (2021).
45. Morales, L., Gambhir, Y., Bennett, J. & Stedman, H. H. Broader Implications of Progressive Liver Dysfunction and Lethal Sepsis in Two Boys following Systemic High-Dose AAV. Mol. Ther. 28, 1753-1755 (2020).
46. Hinderer, C. et al. Severe Toxicity in Nonhuman Primates and Piglets Following High-Dose Intravenous Administration of an Adeno-Associated Virus Vector Expressing Human SMN. Hum. Gene Ther. 29, 285-298 (2018).
47. Kelley, D. R., Snoek, J. & Rinn, J. L. Basset: 1 earning the regulatory code of the accessible genome with deep convolutional neural networks. Genome Res. 26, 990-999 (2016).
48. Cazares, T. A. et al. maxATAC: Genome-scale transcription-factor binding prediction from ATAC-seq with deep neural networks. PLoS Comput. Biol. 19, e1010863 (2023).
49. Locatelli, F. et al. Lentiglobin Gene Therapy for Patients with Transfusion-Dependent B-Thalassemia (TDT): Results from the Phase 3 Northstar-2 and Northstar-3 Studies. Blood 132, 1025 (2018).
50. Locatelli, F. et al. Betibeglogene Autotemcel Gene Therapy for Non-BO/BO Genotype B-Thalassemia. N. Engl. J. Med. 386, 415-427 (2022).
51. Wong, R. L. et al. Lentiviral gene therapy for X-linked chronic granulomatous disease recapitulates endogenous CYBB regulation and expression. Blood 141, 1007-1022 (2023).
52. Kohn, D. B. et al. Lentiviral gene therapy for X-linked chronic granulomatous disease. Nat. Med. 26, 200-206 (2020).
53. Mendell, J. R. et al. Single-Dose Gene-Replacement Therapy for Spinal Muscular Atrophy. N. Engl. J. Med. 377, 1713-1722 (2017).
54. Siders, W. M. et al. Cytotoxic T lymphocyte responses to transgene product, not adeno-associated viral capsid protein, limit transgene expression in mice. Hum. Gene Ther. 20, 11-20 (2009).
55. Tao, N. et al. Sequestration of adenoviral vector by Kupffer cells leads to a nonlinear dose response of transduction in liver. Mol. Ther. 3, 28-35 (2001).
56 Ganesan, L. P. et al. Rapid and efficient clearance of blood-borne virus by liver sinusoidal endothelium. PLoS Pathog. 7, e1002281 (2011).
57. Golovin, D. et al. Google Vizier: A Service for Black-Box Optimization, in Proceedings of the 23rd ACM SIGKDD International Conference on Knowledge Discovery and Data Mining 1487-1495 (Association for Computing Machinery, 2017).
58. Snoek, J., Larochelle, H. & Adams, R. P. Practical bayesian optimization of machine 1 earning algorithms. Adv. Neural Inf. Process. Syst. 25, (2012).
59. Thurman, R. E. et al. The accessible chromatin 1 andscape of the human genome. Nature 489, 75-82 (2012).
60. Zhang, J. et al. An integrative ENCODE resource for cancer genomics. Nat. Commun. 11, 3696 (2020).
61. Hardison, R. C. & Taylor, J. Genomic approaches towards finding cis-regulatory modules in animals. Nat. Rev. Genet. 13, 469-483 (2012).
62. Liu, Y. et al. Functional assessment of human enhancer activities using whole-genome STARR-sequencing. Genome Biol. 18, 219 (2017).
63. Luo, Y. et al. New developments on the Encyclopedia of DNA Elements (ENCODE) data portal. Nucleic Acids Res. 48, D882-D889 (2020).
64. Kagda, M. S. et al. Data navigation on the ENCODE portal. arXiv [q-bio.GN] (2023).
65. Hitz, B. C. et al. The ENCODE Uniform Analysis Pipelines. bioRxiv (2023) doi: 10.1101/2023.04.04.535623.
66. van Laarhoven, P. J. M. & Aarts, E. H. L. Simulated annealing. in Simulated Annealing: Theory and Applications (eds. van Laarhoven, P. J. M. & Aarts, E. H. L.) 7-15 (Springer Netherlands, 1987).
67. Gupta, A., Lal, A., Gunsalus, L., Biancalani, T. & Eraslan, G. Polygraph: A Software Framework for the Systematic Assessment of Synthetic Regulatory DNA Elements. bioRxiv 2023.11.27.568764 (2023) doi: 10.1101/2023.11.27.568764.
68. Sundararajan, M., Taly, A. & Yan, Q. Axiomatic Attribution for Deep Networks. in Proceedings of the 34th International Conference on Machine Learning (eds. Precup, D. & Teh, Y. W.) vol. 70 3319-3328 (PMLR, 06-11 Aug. 2017).
69. Schreiber, J. tfmodisco-lite: A lite implementation of tfmodisco, a motif discovery algorithm for genomics experiments. (Github).
70. Shrikumar, A. et al. Technical Note on Transcription Factor Motif Discovery from Importance Scores (TF-MoDISco) version 0.5.6.5. arXiv [cs.LG] (2018).
71 Castro-Mondragon, J. A. et al. JASPAR 2022: the 9th release of the open-access database of transcription factor binding profiles. Nucleic Acids Res. 50, D165-D173 (2022).
72. Kulakovskiy, I. V. et al. HOCOMOCO: towards a complete collection of transcription factor binding models for human and mouse via large-scale ChIP-Seq analysis. Nucleic Acids Res. 46, D252-D259 (2018).
73. Fulco, C. P. et al. Systematic mapping of functional enhancer-promoter connections with CRISPR interference. Science 354, 769-773 (2016).
74. Parviz, F. et al. Hepatocyte nuclear factor 4alpha controls the development of a hepatic epithelium and liver morphogenesis. Nat. Genet. 34, 292-296 (2003).
75. Harries, L. W., Brown, J. E. & Gloyn, A. L. Species-specific differences in the expression of the HNFIA, HNF1B and HNF4A genes. PLoS One 4, e7855 (2009).
76. El-Khairi, R. & Vallier, L. The role of hepatocyte nuclear factor 1ß in disease and development. Diabetes Obes. Metab. 18 Suppl 1, 23-32 (2016).
77 Odom, D. T. et al. Core transcriptional regulatory circuitry in human hepatocytes. Mol. Syst. Biol. 2, 2006.0017 (2006).
78. Zweidler-Mckay, P. A., Grimes, H. L., Flubacher, M. M. & Tsichlis, P. N. Gfi-1 encodes a nuclear zinc finger protein that binds DNA and functions as a transcriptional repressor. Mol. Cell. Biol. 16, 4024-4034 (1996).
79 Huang, D.-Y., Kuo, Y.-Y. & Chang, Z.-F. GATA-1 mediates auto-regulation of Gfi-1B transcription in K562 cells. Nucleic Acids Res. 33, 5331-5342 (2005).
80. Beauchemin, H. & Moroy, T. Multifaceted Actions of GFI1 and GFI1B in Hematopoietic Stem Cell Self-Renewal and Lineage Commitment. Front. Genet. 11, 591099 (2020).
81. Agoston, Z. & Schulte, D. Meis2 competes with the Groucho co-repressor Tle4 for binding to Otx2 and specifies tectal fate without induction of a secondary midbrain-hindbrain boundary organizer. Development 136, 3311-3322 (2009).
82. Machon, O., Masek, J., Machonova, O., Krauss, S. & Kozmik, Z. Meis2 is essential for cranial and cardiac neural crest development. BMC Dev. Biol. 15, 40 (2015).
83. Zha, Y. et al. MEIS2 is essential for neuroblastoma cell survival and proliferation by transcriptional control of M-phase progression. Cell Death Dis. 5, e1417 (2014).
84. Lee, D. D. & Seung, H. S. Learning the parts of objects by non-negative matrix factorization. Nature 401, 788-791 (1999).
85. Birnbaum, R. Y. et al. Coding exons function as tissue-specific enhancers of nearby genes. Genome Res. 22, 1059-1068 (2012).
86. Kvon, E. Z. et al. Comprehensive In Vivo Interrogation Reveals Phenotypic Impact of Human Enhancer Variants. Cell 180, 1262-1271.e15 (2020).
87. Chatterjee, R. et al. Overlapping ETS and CRE Motifs ((G/C) CGGAAGTGACGTCA (SEQ ID NO: 26)) preferentially bound by GABPa and CREB proteins. G3 2, 1243-1256 (2012).
88. Fornes, O. et al. OnTarget: in silico design of MiniPromoters for targeted delivery of expression. Nucleic Acids Res. 51, W379-W386 (2023).
89. Korecki, A. J. et al. Human MiniPromoters for ocular-rAAV expression in ON bipolar, cone, corneal, endothelial, Müller glial, and PAX6 cells. Gene Ther. 28, 351-372 (2021).
90. Hrvatin, S. et al. A scalable platform for the development of cell-type-specific viral drivers. Elife 8, (2019).
91. Farley, E. K., Olson, K. M., Zhang, W., Rokhsar, D. S. & Levine, M. S. Syntax compensates for poor binding sites to encode tissue specificity of developmental enhancers. Proceedings of the National Academy of Sciences of the United States of America vol. 113 6508-6513 (2016).
92. Farley, E. K. et al. Suboptimization of developmental enhancers. Science 350, 325-328 (2015).
93. Cock, P. J. A. et al. Biopython: freely available Python tools for computational molecular biology and bioinformatics. Bioinformatics 25, 1422-1423 (2009).
94. Quinlan, A. R. & Hall, I. M. BEDTools: a flexible suite of utilities for comparing genomic features. Bioinformatics 26, 841-842 (2010).
95. Dale, R. K., Pedersen, B. S. & Quinlan, A. R. Pybedtools: a flexible Python library for manipulating genomic datasets and annotations. Bioinformatics 27, 3423-3424 (2011).
96. Heinz, S. et al. Simple combinations of lineage-determining transcription factors prime cis-regulatory elements required for macrophage and B cell identities. Mol. Cell 38, 576-589 (2010).
97 Bailey, T. L., Johnson, J., Grant, C. E. & Noble, W. S. The MEME Suite. Nucleic Acids Res. 43, W39-49 (2015).
98. Grant, C. E., Bailey, T. L. & Noble, W. S. FIMO: scanning for occurrences of a given motif. Bioinformatics 27, 1017-1018 (2011).
99. Owen, A. B. & Perry, P. O. Bi-cross-validation of the SVD and the nonnegative matrix factorization. aoas 3, 564-594 (2009).
100.
Kawakami, K. et al. A transposon-mediated gene trap approach identifies developmentally regulated genes in zebrafish. Dev. Cell 7, 133-144 (2004).
101. Levitsky, K. L., Toledo-Aral, J. J., López-Barneo, J. & Villadiego, J. Direct confocal acquisition of fluorescence from X-gal staining on thick tissue sections. Sci. Rep. 3, 2937 (2013).
102. Dobin, A. et al. STAR: ultrafast universal RNA-seq aligner. Bioinformatics 29, 15-21 (2013).
103. Love, M. I., Huber, W. & Anders, S. Moderated estimation of fold change and dispersion for RNA-seq data with DESeq2. Genome Biol. 15, 550 (2014).

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

Further attributes, features, and embodiments of the present invention can be understood by reference to the following numbered aspects of the disclosed invention. Reference to disclosure in any of the preceding aspects is applicable to any preceding numbered aspect and to any combination of any number of preceding aspects, as recognized by appropriate antecedent disclosure in any combination of preceding aspects that can be made. The following numbered aspects are provided:
1. A computer-implemented method to identify or design cis-regulatory elements with cell-type, cell state, tissue type, and/or environment specific activity comprising:
   a. receiving, by one or more computing devices, one or more nucleic acid sequences;
   b. transferring, by one or more computing devices, the one or more nucleic acid sequences to a deployed machine learning network;
   c. processing the one or more nucleic acid sequences with the deployed machine learning network, the deployed machine learning network generated and deployed from a training machine learning network trained on CRE-activity from a massively parallel reporter assay (MPRA) data set that provides empirical cell, tissue, and/or environment specific and/or non-specific MPRA CRE-activity measurements to a model,
   d. generating, by the deployed machine learning network, a prediction of a CRE activity of the one or more nucleic acid sequences; and
   e. transmitting, by one or more computing devices, the predicted CRE activity to a user device associated with a user.
2. The method of aspect 1, wherein the CRE activity is cell type, cell state, tissue type, or environment specific MPRA CRE-activity.
3. The method of any one of aspects 1-2, wherein the one or more nucleic acid sequences is a genome or a portion thereof or an epigenome or portion thereof.
4. The method of any one of aspects 1-2, wherein the one or more nucleic acid sequences is a DNA sequence generated from a suitable DNA sequence generation algorithm, optionally evolutionary, probabilistic, simulated annealing, or gradient based updates with random momentum (GRUM).
5. The method of any one of aspects 1-4, wherein processing further comprises iterative cell, tissue, or environment specific regulatory optimization of the one or more nucleic acid sequences, wherein iterative cell, tissue, or environment specific regulatory optimization comprises sequentially modifying the one or more nucleic acid sequences in each iteration.
6. The method of any one of aspects 1-5, wherein processing further comprises passing the prediction to a cell, tissue, or environment specific regulatory optimizing objective function that maximizes cell specific regulatory activity.
7. The method of aspect 6, wherein the cell specific regulatory optimizing objective function maximizes a predicted expression of a given sequence in one cell type, cell state, tissue type, or environment while reducing expression in all other cell types, cell states, tissue types, or environments.
8. The method of aspect 6 or 7, further comprising updating the one or more nucleic acid sequences in each iteration based on an output of the cell, tissue, or environment specific regulatory optimizing objective function.
9. The method of any one of aspects 6-8, wherein the objective function prioritizes nucleic acid sequences with cell type, cell state, tissue type, or environment specific promoter activity, enhancer activity, silencer activity, or insulator activity.
10. The method of any one of aspects 6-8, wherein the cell type, cell state, tissue type, or environment specific regulatory activity comprises promoter activity, enhancer activity, silencer activity, or insulator activity.
11. The method of any of aspects 1-10, wherein the machine learning network comprises a neural network, Bayesian network, random forest, matrix factorization, hidden Markov model, support vector machine, K-means clustering, K-nearest neighbor, linear classifiers, logistic classifiers, or any combination thereof.
12. The method of aspect 11, wherein the neural network comprises deep learning, a convolutional neural network, or a recurrent neural network.
13. The method of aspect 12, wherein the neural network comprises the convolutional neural network.
14. The method of any one of aspects 1-13, wherein the cell, tissue, or environment specific CRE-activity MPRA data set is obtained from a suitable database, optionally CREs centered on variants from the UK Biobank and/or GTEx.
15. The method of any one of aspects 1-14, wherein the cell type, cell state, tissue type, or environment specific CRE-activity MPRA data set comprises a plurality of pairs of reference and alternate alleles.
16. The method of any one of aspects 1-15, wherein the cell, tissue, or environment specific engineered CREs are cell type, cell state, tissue type, or environment specific engineered CREs.
17. The method of any one of aspects 1-16, wherein the cell type, cell state, tissue type, or environment specific CRE-activity MPRA data set was generated using vertebrate cells or invertebrate cells.
18. The method of any one of aspects 1-17, wherein the cell type, cell state, tissue type, or environment specific CRE-activity MPRA data set was generated using mammalian, avian, reptilian, fish, or amphibian cells.
19. The method of any one of aspects 1-18, wherein the cell type, cell state, tissue type, or environment specific CRE-activity MPRA data set was generated using human or non-human primate cells.
20. The method of any one of aspects 1-19, wherein the cell type, cell state, tissue type, or environment specific CRE-activity MPRA data set was generated using plant cells.
21. The method of any one of aspects 1-20, wherein the one or more nucleic acid sequence is 200 bases or less.
22. The method of any one of aspects 1-21, wherein the training machine learning network comprises unsupervised learning, supervised learning, semi-supervised learning, reinforcement learning, transfer learning, incremental learning, curriculum learning, learning to learn, contrastive learning, or any combination thereof.
23. A system to identify or design cis-regulatory elements with cell-type, cell state, tissue type, and/or environment specific activity, comprising:
   a storage device; and
   a processor communicatively coupled to the storage device, wherein the processor executes application code instructions that are stored in the storage device to cause the system to:
   a. receive, by one or more computing devices, one or more nucleic acid sequences;
   b. transfer, by one or more computing devices, the one or more nucleic acid sequences to a deployed machine learning network;
   c. process the one or more nucleic acid sequences with the deployed machine learning network, the deployed machine learning network generated and deployed from a training machine learning network trained on CRE-activity from a massively parallel reporter assay (MPRA) data set that provides empirical cell, tissue, or environment specific and non-specific MPRA CRE-activity measurements to a model,
   d. generate, by the deployed machine learning network, a prediction of a CRE activity of the one or more nucleic acid sequences; and
   e. transmit, by one or more computing devices, the predicted CRE activity to a user device associated with a user.
24. The system of aspect 23, wherein the CRE activity is cell type, cell state, tissue type, or environment specific MPRA CRE-activity.
25. The system of any one of aspects 23-24, wherein the one or more nucleic acid sequences is a genome or a portion thereof or an epigenome or portion thereof.
26. The system of any one of aspects 23-24, wherein the one or more nucleic acid sequence is a DNA sequence generated from a suitable DNA sequence generation algorithm, optionally evolutionary, probabilistic, simulated annealing, or gradient based updates with random momentum (GRUM).
27. The system of any one of aspects 23-26, wherein processing further comprises iterative cell, tissue, or environment specific regulatory optimization of the one or more nucleic acid sequence, wherein iterative cell, tissue, or environment specific regulatory optimization comprises sequentially modifying the nucleic acid sequence in each iteration.
28. The system of any one of aspects 23-27, wherein processing further comprises passing the prediction to a cell, tissue, or environment specific regulatory optimizing objective function that maximizes cell specific regulatory activity.
29. The system of aspect 28, wherein the cell specific regulatory optimizing objective function maximizes a predicted expression of a given sequence in one cell type, cell state, tissue type, or environment while reducing expression in all other cell types, cell states, tissue types, or environments.
30. The system of aspect 28 or 29, further comprising updating the one or more nucleic acid sequences in each iteration based on an output of the cell, tissue, or environment specific regulatory optimizing objective function.
31. The system of any one of aspects 28-30, wherein the objective function prioritizes nucleic acid sequences with cell type, cell state, tissue type, or environment specific promoter activity, enhancer activity, silencer activity, or insulator activity.
32. The system of any one of claim aspects 28-30, wherein the cell type, cell state, tissue type, or environment specific regulatory activity comprises promoter activity, enhancer activity, silencer activity, or insulator activity.
33. The system of any of aspects 23-32, wherein the machine learning network comprises a neural network, Bayesian network, random forest, matrix factorization, hidden Markov model, support vector machine, K-means clustering, K-nearest neighbor, linear classifiers, logistic classifiers, or any combination thereof.
34. The system of aspect 33, wherein the neural network comprises deep learning, a convolutional neural network, or a recurrent neural network.
35. The system of aspect 34, wherein the neural network comprises the convolutional neural network.
36. The system of any one of aspects 23-35, wherein the cell, tissue, or environment specific CRE-activity MPRA data set is obtained from a suitable database, optionally CREs centered on variants from the UK Biobank and/or GTEx.
37. The system of any one of aspects 23-36, wherein the cell type, cell state, tissue type, or environment specific CRE-activity MPRA data set comprises a plurality of pairs of reference and alternate alleles.
38. The system of any one of aspects 23-37, wherein the cell, tissue, or environment specific engineered CREs are cell type, cell state, tissue type, or environment specific engineered CREs.
39. The system of any one of aspects 23-38, wherein the cell type, cell state, tissue type, or environment specific CRE-activity MPRA data set was generated using vertebrate cells or invertebrate cells.
40. The system of any one of aspects 23-39, wherein the cell type, cell state, tissue type, or environment specific CRE-activity MPRA data set was generated using mammalian, avian, reptilian, fish, or amphibian cells.
41. The system of any one of aspects 23-40, wherein the cell type, cell state, tissue type, or environment specific CRE-activity MPRA data set was generated using human or non-human primate cells.
42. The system of any one of aspects 23-41, wherein the cell type, cell state, tissue type, or environment specific CRE-activity MPRA data set was generated using plant cells.
43. The system of any one of claims 23-42, wherein the one or more nucleic acid sequence is 200 bases or less.
44. The system of any one of aspects 23-43, wherein the training machine learning network comprises unsupervised learning, supervised learning, semi-supervised learning, reinforcement learning, transfer learning, incremental learning, curriculum learning, learning to learn, contrastive learning, or any combination thereof.
45. A computer program product, comprising:
   a non-transitory computer-readable storage device having computer-executable program instructions embodied thereon that when executed by a computer cause the computer to identify or design cis-regulatory elements with cell-type, cell state, tissue type, and/or environment specific activity, the computer-executable program instructions comprising:
   a. computer-executable program instructions to receive, by one or more computing devices, one or more nucleic acid sequences;
   b. computer-executable program instructions to transfer, by one or more computing devices, the one or more nucleic acid sequences to a deployed machine learning network;
   c. computer-executable program instructions to process the one or more nucleic acid sequences with the deployed machine learning network, the deployed machine learning network generated and deployed from a training machine learning network trained on CRE-activity from a massively parallel reporter assay (MPRA) data set that provides empirical cell, tissue, or environment specific and non-specific MPRA CRE-activity measurements to a model,
   d. computer-executable program instructions to generate, by the deployed machine learning network, a prediction of a CRE activity of the one or more nucleic acid sequences; and
   e. computer-executable program instructions to transmit, by one or more computing devices, the predicted CRE activity to a user device associated with a user.
46. The computer program product of aspect 45, wherein the CRE activity is cell type, cell state, tissue type, or environment specific MPRA CRE-activity.
47. The computer program product of any one of aspects 45-46, wherein the one or more nucleic acid sequences is a genome or a portion thereof or an epigenome or portion thereof.
48. The computer program product of aspect 45-46, wherein the one or more nucleic acid sequence is a DNA sequence generated from a suitable DNA sequence generation algorithm, optionally evolutionary, probabilistic, simulated annealing, or gradient based updates with random momentum (GRUM).
49. The computer program product of any one of aspects 45-48, wherein processing further comprises iterative cell, tissue, or environment specific regulatory optimization of the one or more nucleic acid sequence, wherein iterative cell, tissue, or environment specific regulatory optimization comprises sequentially modifying the nucleic acid sequence in each iteration.
50. The computer program product of any one of aspects 45-49, wherein processing further comprises passing the prediction to a cell, tissue, or environment specific regulatory optimizing objective function that maximizes cell specific regulatory activity.
51. The computer program product of aspect 50, wherein the cell specific regulatory optimizing objective function maximizes a predicted expression of a given sequence in one cell type, cell state, tissue type, or environment while reducing expression in all other cell types, cell states, tissue types, or environments.
52. The computer program product of aspect 50 or 51, further comprising updating the one or more nucleic acid sequences in each iteration based on an output of the cell, tissue, or environment specific regulatory optimizing objective function.
53. The computer program product of any one of aspects 50-52, wherein the objective function prioritizes nucleic acid sequences with cell type, cell state, tissue type, or environment specific promoter activity, enhancer activity, silencer activity, or insulator activity.
54. The computer program product of any one of aspects 50-52, wherein the cell type, cell state, tissue type, or environment specific regulatory activity comprises promoter activity, enhancer activity, silencer activity, or insulator activity.
55. The computer program product of any of aspects 45-54, wherein the machine learning network comprises a neural network, Bayesian network, random forest, matrix factorization, hidden Markov model, support vector machine, K-means clustering, K-nearest neighbor, linear classifiers, logistic classifiers, or any combination thereof.
56. The computer program product of aspect 55, wherein the neural network comprises deep learning, a convolutional neural network, or a recurrent neural network.
57. The computer program product of aspect 56, wherein the neural network comprises the convolutional neural network.
58. The computer program product of any one of aspects 45-57, wherein the cell, tissue, or environment specific CRE-activity MPRA data set is obtained from a suitable database, optionally CREs centered on variants from the UK Biobank and/or GTEx.
59. The computer program product of any one of aspects 45-58, wherein the cell type, cell state, tissue type, or environment specific CRE-activity MPRA data set comprises a plurality of pairs of reference and alternate alleles.
60. The computer program product of any one of aspects 45-59, wherein the cell, tissue, or environment specific engineered CREs are cell type, cell state, tissue type, or environment specific engineered CREs.
61. The computer program product of any one of aspects 45-60, wherein the cell type, cell state, tissue type, or environment specific CRE-activity MPRA data set was generated using vertebrate cells or invertebrate cells.
62. The computer program product of any one of aspects 45-61, wherein the cell type, cell state, tissue type, or environment specific CRE-activity MPRA data set was generated using mammalian, avian, reptilian, fish, or amphibian cells.
63. The computer program product of any one of aspects 45-62, wherein the cell type, cell state, tissue type, or environment specific CRE-activity MPRA data set was generated using human or non-human primate cells.
64. The computer program product of any one of aspects 45-63, wherein the cell type, cell state, tissue type, or environment specific CRE-activity MPRA data set was generated using plant cells.
65. The computer program product of any one of aspects 45-64, wherein the one or more nucleic acid sequence is 200 bases or less.
66. The computer program product of any one of aspects 45-65, wherein the training machine learning network comprises unsupervised learning, supervised learning, semi-supervised learning, reinforcement learning, transfer learning, incremental learning, curriculum learning, learning to learn, contrastive learning, or any combination thereof.
67. A cis-regulatory element (CRE), wherein the CRE is identified or designed using a method as in any one of aspects 1-21, optionally wherein the CRE is an engineered CRE.
68. The CRE of aspect 67, wherein the CRE comprises two or more CREs designed using a method as in any one of aspects 1-21, optionally where one or more of the two or more CREs are an engineered CRE.
69. The engineered CRE of any one of aspects 67-68 wherein the engineered CRE is cell type, cell state, tissue type, and/or environment specific.
70. The engineered CRE of any one of aspects 67-69, wherein the engineered CRE does not have a significant match in a genome of an organism.
71. The engineered CRE of aspect 70, wherein the organism is a vertebrate or invertebrate.
72. The engineered CRE of any one of aspects 70-71, wherein the organism is a mammal, avian, reptile, fish, or amphibian.
73. The engineered CRE of any one of aspects 70-72, wherein the organism is a human or non-human primate.
74. The engineered CRE of aspect 70, wherein the organism is a plant.
75. The CRE, optionally engineered CRE, of any one of aspects 67-74, wherein the CRE is specific for a diseased or abnormal cell type and/or cell state.
76. An engineered therapeutic polynucleotide comprising:
   a CRE, optionally an engineered CRE, of any one of aspects 1-75; and
   a therapeutic polynucleotide, wherein the CRE is operatively coupled to the therapeutic polynucleotide.
77. The engineered therapeutic polynucleotide of aspect 76, wherein the therapeutic polynucleotide
   a. comprises a replacement gene;
   b. encodes a therapeutic gene product;
   c. comprises or encodes a genetic modification system or component thereof;
   d. comprises or encodes an RNAi molecule;
   e. comprises or encodes an aptamer;
   f. any combination of (a)-(e).
78. An engineered reporter polynucleotide comprising:
   a CRE, optionally an engineered CRE, of any one of aspects 67-75; and
   a reporter polynucleotide, wherein the reporter polynucleotide is operatively coupled to the CRE.
79. The engineered reporter polynucleotide of aspect 78, wherein expression of the reporter polynucleotide produces a detectable signal.
80. The engineered reporter polynucleotide of aspect 79, wherein the reporter polynucleotide
   a. encodes a reporter gene product;
   b. comprises or encodes a genetic modification system or component thereof;
   c. comprises a transcribable barcode;
   d. comprises a DNA barcode;
   e. comprises a target sequence for a sequence-specific binding molecule or system;
   f. comprises a DNA origami reporter system or a component thereof;
   g. comprises or encodes an RNAi molecule;
   h. comprises or encodes an aptamer;
   i. or any combination of (a)-(h).
81. A vector comprising a CRE as in any one of aspects 67-75.
82. A vector comprising an engineered therapeutic polynucleotide and/or an engineered reporter polynucleotide of any one of aspects 76-80.
83. A delivery vehicle comprising an engineered therapeutic polynucleotide and/or an engineered reporter polynucleotide of any one of aspects 76-80 and/or a vector as in any one of aspects 84-85.
84. A cell comprising:
   a. the engineered therapeutic polynucleotide and/or an engineered reporter polynucleotide of any one of aspects 76-80;
   b. the vector of any one of aspects 81-82,
   c. the delivery vehicle of aspect 83; or
   d. any combination of (a)-(c).
85. A pharmaceutical formulation comprising:
   a. the engineered therapeutic polynucleotide and/or an engineered reporter polynucleotide of any one of aspects 76-80;
   b. the vector of any one of aspects 81-82,
   c. the delivery vehicle of aspect83;
   d. the cell of aspect 84; or
   e. any combination of (a)-(d); and
      a pharmaceutically acceptable carrier.
86. A device configured to detect a specific cell type and/or cell state of one or more cells comprising:
   an engineered reporter polynucleotide of any one of aspects 78-80 and/or a delivery vehicle comprising the same.
87. The device of aspect 86, wherein the device comprises microfluidic device, a lateral flow device, a tangential flow device, a normal flow device, a micro-electromechanical system, or any combination thereof.
88. The device of any one of aspects 86-87, further comprising a detection reagent, wherein the detection reagent comprises a sequence-specific binding molecule or system capable of specifically binding the reporter polynucleotide, optionally at a target sequence for a sequence-specific binding molecule or system.
89. The device of aspect 88, wherein the sequence-specific binding molecule or system comprises a programmable nuclease or system thereof, optionally wherein the programmable nuclease or system thereof is a Cas or Cas-based system, or an OMEGA system.
90. A method of detecting a specific cell type, cell state, tissue type, and/or environment of one or more cells in a sample comprising:
   delivering to one or more cells an engineered reporter polynucleotide of any one of aspects 78-80 and/or a delivery vehicle comprising the same under conditions sufficient for expression of the engineered reporter polynucleotide,
   wherein expression of the reporter polynucleotide occurs substantially only in the specific cell type, cell state, tissue type, and/or environment in which the CRE is active in.
91. The method of aspect 90, wherein expression of the reporter polynucleotide generates a detectable signal.
92. The method of aspect 90, further comprising contacting the one or more cells with a detection reagent, wherein the detection reagent comprises a sequence-specific binding molecule or system capable of specifically binding the reporter polynucleotide, optionally at the target sequence for a sequence-specific binding molecule or system.
93. The method of aspect 92, wherein the sequence-specific binding molecule or system comprises a programmable nuclease or system thereof, optionally wherein the programmable nuclease or system thereof is a Cas or Cas-based system or an OMEGA system.
94. The method of any one of aspects 92-93, wherein binding of the sequence-specific binding molecule or system to specifically binding the reporter polynucleotide produces a detectable signal.
95. The method of aspect 90 or 94, further comprising detecting the detectable signal.
96. The method of aspect 95, wherein the detectable signal indicates a specific cell type, cell state, tissue type, and/or environment.
97. The method of any one of aspects 95-96, wherein the detectable signal is an optical signal, a genetic perturbation, a change in gene expression of a target gene, expression of a barcode, change in genotype, change in phenotype, or any combination thereof.
98. The method of aspect 97, wherein detecting comprises optical detection of the detectable signal, DNA sequencing, RNA sequencing, a hybridization-based gene expression analysis, mass-spectrometry, immunodetection, or any combination thereof.
99. The method of any one of aspects 97-98, wherein detecting comprises a single-cell resolved assay.
100. The method of any one of aspects 90-99, wherein the sample comprises a biofluid optionally selected from saliva, urine, blood or portion thereof, sweat, milk, semen, lymph, mucus, or feces.
101 The method of any one of aspects 90-99, wherein the sample comprises a tissue or portion thereof.
102. The method of any one of aspects 90-99, wherein the method comprises in situ spatial detection of expression of the reporter polynucleotide.
103. The method of any one of aspects 90-102, wherein one or more of the steps of the method are performed in vitro, in vivo, in situ, or ex vivo.
104. A method of cell type, cell state, tissue type, and/or environment specific delivery of a therapeutic polynucleotide comprising:
   delivering to one or more cells an engineered therapeutic polynucleotide of any one of aspects 76-77, a delivery vehicle comprising the same, or a pharmaceutical formulation thereof under conditions sufficient for expression of the engineered therapeutic polynucleotide.
105. The method of aspect 104, wherein expression of the therapeutic polynucleotide occurs substantially only in a specific cell type, cell state, tissue type, and/or environment in which the CRE is active in.
106. The method of any one of aspects 104-105, wherein delivering occurs in vivo or ex vivo.
107. The method of any one of aspects 104-105, wherein the one or more cells are present in a subject in need thereof.
108. The method of any one of aspects 104-107, wherein delivery is systemic or local.
109. The method of any one of aspects 104-108, wherein the one or more cells are delivered to a subject in need thereof after delivering to the one or more cells an engineered therapeutic polynucleotide of any one of aspects 78-79, a delivery vehicle comprising the same, or a pharmaceutical formulation thereof.
110. The method of aspect 109, wherein the one or more cells allogenic to the subject in need thereof or are autologous.
111. A method of treating a disease or disorder or a symptom thereof in a subject in need thereof comprising:
   delivering to one or more cells of the subject in need thereof an engineered therapeutic polynucleotide of any one of aspects 76-77, a delivery vehicle comprising the same, or a pharmaceutical formulation thereof under conditions sufficient for expression of the engineered therapeutic polynucleotide.
112. The method of aspect 111, wherein expression of the therapeutic polynucleotide occurs substantially only in a specific cell type, cell state, tissue type, and/or environment in which the CRE is active in.
113. The method of any one of aspects 111-112, wherein delivering occurs in vivo or ex vivo.
114. The method of any one of aspects 111-113, wherein delivery is systemic or local.
115. The method of any one of aspects 104-114, further comprising delivering the one or more cells to the subject in need thereof after delivering to the one or more cells an engineered therapeutic polynucleotide of any one of claims 76-77, a delivery vehicle comprising the same, or a pharmaceutical formulation thereof.
116. The method of any one of aspects 104-115, wherein the therapeutic polynucleotide (a) generates one or more genetic or epigenetic mutations, (b) generates a replacement gene product, (c) modulates gene and/or gene product expression, (d) kills or inhibits the growth or infection by a pathogen, (e) modulates one or more cellular activities, functions, or interactions, (f) kills or inhibits cell growth, differentiation, and/or proliferation, or (g) any combination of (a)-(f) in/of the one or more cells in which the therapeutic polynucleotide is expressed.
117. The method of any one of aspects 90-116, wherein the one or more cells comprises or consists of vertebrate cells or invertebrate cells.
118. The method of any one of aspects 90-117, wherein the one or more cells comprises or consists of mammalian, avian, reptilian, fish, amphibian cells, or insect cells.
119. The method of any one of aspects 90-118, wherein the one or more cells comprises or consists of human or non-human primate cells.
120. The method of any one of aspects 90-116, wherein the one or more cells comprises or consists of plant cells.
121. The method of any one of aspects 90-116, wherein the one or more cells comprises or consists of prokaryotic cells.
122. The method of any one of aspects 107-116, wherein the subject in need thereof is a vertebrate or invertebrate.
123. The method of aspect 122, wherein the subject in need thereof is a mammal, avian, reptile, fish, amphibian, or insect.
124. The method of any one of aspects 121-123, wherein the subject in need thereof is a human or non-human primate.
125. The method of any one of aspects 107-116, wherein the one or more cells comprises or consists of plant cells.

1. A computer-implemented method to identify or design cis-regulatory elements with cell-type, cell state, tissue type, and/or environment specific activity comprising:
a receiving, by one or more computing devices, one or more nucleic acid sequences;
b. transferring, by one or more computing devices, the one or more nucleic acid sequences to a deployed machine learning network;
c. processing the one or more nucleic acid sequences with the deployed machine learning network, the deployed machine learning network generated and deployed from a training machine learning network trained on CRE-activity from a massively parallel reporter assay (MPRA) data set that provides empirical cell, tissue, and/or environment specific and/or non-specific MPRA CRE-activity measurements to a model,
d. generating, by the deployed machine learning network, a prediction of a CRE activity of the one or more nucleic acid sequences; and
e. transmitting, by one or more computing devices, the predicted CRE activity to a user device associated with a user.

2. The method of claim 1, wherein the CRE activity is cell type, cell state, tissue type, or environment specific MPRA CRE-activity.

3. The method of claim 1, wherein the one or more nucleic acid sequences is a genome or a portion thereof or an epigenome or portion thereof.

4. The method of claim 1, wherein the one or more nucleic acid sequences is a DNA sequence generated from a suitable DNA sequence generation algorithm, optionally evolutionary, probabilistic, simulated annealing, or gradient based updates with random momentum (GRUM).

5. The method of claim 1, wherein processing further comprises iterative cell, tissue, or environment specific regulatory optimization of the one or more nucleic acid sequences, wherein iterative cell, tissue, or environment specific regulatory optimization comprises sequentially modifying the one or more nucleic acid sequences in each iteration.

6. The method of claim 1, wherein processing further comprises passing the prediction to a cell, tissue, or environment specific regulatory optimizing objective function that maximizes cell specific regulatory activity.

7. The method of claim 6, wherein the cell specific regulatory optimizing objective function maximizes a predicted expression of a given sequence in one cell type, cell state, tissue type, or environment while reducing expression in all other cell types, cell states, tissue types, or environments.

8. The method of claim 6, further comprising updating the one or more nucleic acid sequences in each iteration based on an output of the cell, tissue, or environment specific regulatory optimizing objective function.

9. The method of claim 6, wherein the objective function prioritizes nucleic acid sequences with cell type, cell state, tissue type, or environment specific promoter activity, enhancer activity, silencer activity, or insulator activity.

10. The method of claim 6, wherein the cell type, cell state, tissue type, or environment specific regulatory activity comprises promoter activity, enhancer activity, silencer activity, or insulator activity.

11. The method of claim 1, wherein the machine learning network comprises a neural network, Bayesian network, random forest, matrix factorization, hidden Markov model, support vector machine, K-means clustering, K-nearest neighbor, linear classifiers, logistic classifiers, or any combination thereof.

12. The method of claim 11, wherein the neural network comprises deep learning, a convolutional neural network, or a recurrent neural network.

13. The method of claim 12, wherein the neural network comprises the convolutional neural network.

14. The method of claim 1, wherein the cell, tissue, or environment specific CRE-activity MPRA data set is obtained from a suitable database, optionally CREs centered on variants from the UK Biobank and/or GTEx.

15. The method of claim 1, wherein the cell type, cell state, tissue type, or environment specific CRE-activity MPRA data set comprises a plurality of pairs of reference and alternate alleles.

16. The method of claim 1, wherein the cell, tissue, or environment specific engineered CREs are cell type, cell state, tissue type, or environment specific engineered CREs.

17. The method of claim 1, wherein the cell type, cell state, tissue type, or environment specific CRE-activity MPRA data set was generated using vertebrate cells or invertebrate cells.

18. The method of claim 1, wherein the cell type, cell state, tissue type, or environment specific CRE-activity MPRA data set was generated using mammalian, avian, reptilian, fish, or amphibian cells.

19. The method of claim 1, wherein the cell type, cell state, tissue type, or environment specific CRE-activity MPRA data set was generated using human or non-human primate cells.

20. The method of claim 1, wherein the cell type, cell state, tissue type, or environment specific CRE-activity MPRA data set was generated using plant cells.

21. The method of claim 1, wherein the one or more nucleic acid sequence is 200 bases or less.

22. The method of claim 1, wherein the training machine learning network comprises unsupervised learning, supervised learning, semi-supervised learning, reinforcement learning, transfer learning, incremental learning, curriculum learning, learning to learn, contrastive learning, or any combination thereof.

23. A system to identify or design cis-regulatory elements with cell-type, cell state, tissue type, and/or environment specific activity, comprising:
a storage device; and
a processor communicatively coupled to the storage device, wherein the processor executes application code instructions that are stored in the storage device to cause the system to:
a receive, by one or more computing devices, one or more nucleic acid sequences;
b. transfer, by one or more computing devices, the one or more nucleic acid sequences to a deployed machine learning network;
c. process the one or more nucleic acid sequences with the deployed machine learning network, the deployed machine learning network generated and deployed from a training machine learning network trained on CRE-activity from a massively parallel reporter assay (MPRA) data set that provides empirical cell, tissue, or environment specific and non-specific MPRA CRE-activity measurements to a model,
d. generate, by the deployed machine learning network, a prediction of a CRE activity of the one or more nucleic acid sequences; and
e. transmit, by one or more computing devices, the predicted CRE activity to a user device associated with a user.

24. The system of claim 23, wherein the CRE activity is cell type, cell state, tissue type, or environment specific MPRA CRE-activity.

25. The system of claim 23, wherein the one or more nucleic acid sequences is a genome or a portion thereof or an epigenome or portion thereof, or a DNA sequence generated from a suitable DNA sequence generation algorithm, optionally evolutionary, probabilistic, simulated annealing, or gradient based updates with random momentum (GRUM).

26. (canceled)

27. The system of claim 23, wherein processing comprises:
a) iterative cell, tissue, or environment specific regulatory optimization of the one or more nucleic acid sequence, wherein iterative cell, tissue, or environment specific regulatory optimization comprises sequentially modifying the nucleic acid sequence in each iteration; and
b) processing further comprises passing the prediction to a cell, tissue, or environment specific regulatory optimizing objective function that maximizes cell specific regulatory activity, wherein the objective function optionally:
i) maximizes a predicted expression of a given sequence in one cell type, cell state, tissue type, or environment while reducing expression in all other cell types, cell states, tissue types, or environments;
ii) prioritizes nucleic acid sequences with cell type, cell state, tissue type, or environment specific promoter activity, enhancer activity, silencer activity, or insulator activity:
c) and further comprising updating the one or more nucleic acid sequences in each iteration based on an output of the cell, tissue, or environment specific regulatory optimizing objective function.

28. (canceled)

29. (canceled)

30. (canceled)

31. (canceled)

32. (canceled)

33. The system of claim 23, wherein the machine learning network comprises a neural network, Bayesian network, random forest, matrix factorization, hidden Markov model, support vector machine, K-means clustering, K-nearest neighbor, linear classifiers, logistic classifiers, or any combination thereof, optionally wherein the neural network comprises deep learning, a convolutional neural network, or a recurrent neural network.

34. (canceled)

35. (canceled)

36. The system of claim 23, wherein the cell, tissue, or environment specific CRE-activity MPRA data set is obtained from a suitable database, optionally CREs centered on variants from the UK Biobank and/or GTEx, and optionally wherein the MPRA data set comprises a plurality of pairs of reference and alternate alleles.

37. (canceled)

38. The system of claim 23, wherein the cell, tissue, or environment specific engineered CREs are cell type, cell state, tissue type, or environment specific engineered CREs.

39. The system of claim 23, wherein the cell type, cell state, tissue type, or environment specific CRE-activity MPRA data set was generated using cells selected from: vertebrate cells invertebrate cells, mammalian cells, avian cells, reptilian cells, fish cells, amphibian cells, insect cells, human cells, non-human primate cells, or plant cells.

40. (canceled)

41. (canceled)

42. (canceled)

43. The system of claim 23, wherein the one or more nucleic acid sequence is 200 bases or less; and the training machine learning network comprises unsupervised learning, supervised learning, semi-supervised learning, reinforcement learning, transfer learning, incremental learning, curriculum learning, learning to learn, contrastive learning, or any combination thereof.

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. (canceled)

58. (canceled)

59. (canceled)

60. (canceled)

61. (canceled)

62. (canceled)

63. (canceled)

64. (canceled)

65. (canceled)

66. (canceled)

67. A cis-regulatory element (CRE), wherein the CRE is identified or designed using a system as in claim 23, optionally wherein the CRE is an engineered CRE.

68. The CRE of claim 67, wherein the CRE comprises two or more CREs designed using a system as in claim 23, optionally where one or more of the two or more CREs are an engineered CRE.

69. The engineered CRE of claim 67, wherein the engineered CRE is cell type, cell state, tissue type, and/or environment specific.

70. The engineered CRE of claim 67, wherein the engineered CRE does not have a significant match in a genome of an organism selected from: vertebrate, invertebrate, mammal, avian, reptile, fish, amphibian, human, non-human primate, or plant.

71. (canceled)

72. (canceled)

73. (canceled)

74. (canceled)

75. The CRE, optionally engineered CRE, of claim 67, wherein the CRE is specific for a diseased or abnormal cell type and/or cell state.

76. An engineered therapeutic polynucleotide comprising:
a CRE, optionally an engineered CRE, of claim 67; and
a therapeutic polynucleotide, wherein the CRE is operatively coupled to the therapeutic polynucleotide.

77. The engineered therapeutic polynucleotide of claim 76, wherein the therapeutic polynucleotide
a. comprises a replacement gene;
b. encodes a therapeutic gene product;
c. comprises or encodes a genetic modification system or component thereof;
d. comprises or encodes an RNAi molecule;
e. comprises or encodes an aptamer;
f. any combination of (a)-(e).

78. An engineered reporter polynucleotide comprising:
a CRE, optionally an engineered CRE, of any one of claim 67; and
a reporter polynucleotide, wherein the reporter polynucleotide is operatively coupled to the CRE, wherein expression of the reporter polynucleotide produces a detectable signal.

79. (canceled)

80. The engineered reporter polynucleotide of claim 78, wherein the reporter polynucleotide
a. encodes a reporter gene product;
b. comprises or encodes a genetic modification system or component thereof;
c. comprises a transcribable barcode;
d. comprises a DNA barcode;
e. comprises a target sequence for a sequence-specific binding molecule or system;
f. comprises a DNA origami reporter system or a component thereof;
g. comprises or encodes an RNAi molecule;
h. comprises or encodes an aptamer;
i. or any combination of (a)-(h).

81. A vector or delivery vehicle comprising:
a CRE as in claim 67;
an engineered therapeutic polynucleotide and/or an engineered reporter polynucleotide of claim 76;
an engineered reporter polynucleotide of claim 78; or
any combination thereof.

82. (canceled)

83. (canceled)

84. (canceled)

85. (canceled)

86. (canceled)

87. (canceled)

88. (canceled)

89. (canceled)

90. A method of detecting a specific cell type, cell state, tissue type, and/or environment of one or more cells in a sample comprising:
delivering to one or more cells an engineered reporter polynucleotide of any one of claims 80-82 and/or a delivery vehicle comprising the same under conditions sufficient for expression of the engineered reporter polynucleotide,
wherein expression of the reporter polynucleotide occurs substantially only in the specific cell type, cell state, tissue type, and/or environment in which the CRE is active in; and
optionally wherein the method further comprises:
contacting the one or more cells with a detection reagent comprising a sequence-specific binding molecule or system capable of specifically binding the reporter polynucleotide, optionally wherein the sequence-specific binding molecule or system comprises a programmable nuclease or system thereof (optionally a Cas or Cas-based system, IscB or IscB system, or OMEGA system), and optionally wherein binding produces a detectable signal.

91. The method of claim 90, wherein expression of the reporter polynucleotide generates a detectable signal.

92. (canceled)

93. (canceled)

94. (canceled)

95. The method of claim 90, further comprising detecting the detectable signal, wherein
the detectable signal indicates a specific cell type, cell state, tissue type, and/or environment;
the detectable signal is an optical signal, a genetic perturbation, a change in gene expression of a target gene, expression of a barcode, change in genotype, change in phenotype, or any combination thereof; and
detecting comprises optical detection of the detectable signal, DNA sequencing, RNA sequencing, a hybridization-based gene expression analysis, mass-spectrometry, immunodetection, single-cell resolved assay, or any combination thereof.

96. (canceled)

97. (canceled)

98. (canceled)

99. (canceled)

100. The method of claim 90, wherein;
the sample comprises a biofluid optionally selected from saliva, urine, blood or portion thereof, sweat, milk, semen, lymph, mucus, or feces; or
the sample comprises a tissue or portion thereof; or
the method comprises in situ spatial detection of expression of the reporter polynucleotide.

101. (canceled)

102. (canceled)

103. The method of claim 90, wherein one or more of the steps of the method are performed in vitro, in vivo, in situ, or ex vivo.

104. A method of cell type, cell state, tissue type, and/or
environment specific delivery of a therapeutic polynucleotide comprising:
delivering to one or more cells an engineered therapeutic polynucleotide of any one of claim 76, a delivery vehicle comprising the same, or a pharmaceutical formulation thereof under conditions sufficient for expression of the engineered therapeutic polynucleotide.

105. The method of claim 104, wherein;
expression of the therapeutic polynucleotide occurs substantially only in a specific cell type, cell state, tissue type, and/or environment in which the CRE is active in;
delivering occurs in vivo or ex vivo;
the one or more cells are present in a subject in need thereof;
delivery is systemic or local; and
the one or more cells are optionally delivered to a subject in need thereof after delivering the engineered therapeutic polynucleotide, wherein the one or more cells are allogenic to the subject or are autologous.

106. (canceled)

107. (canceled)

108. (canceled)

109. (canceled)

110. (canceled)

111. A method of treating a disease or disorder or a symptom thereof in a subject in need thereof comprising:
delivering to one or more cells of the subject in need thereof an engineered therapeutic polynucleotide of claim 76, a delivery vehicle comprising the same, or a pharmaceutical formulation thereof under conditions sufficient for expression of the engineered therapeutic polynucleotide.

112. The method of claim 111, wherein;
expression of the therapeutic polynucleotide occurs substantially only in a specific cell type, cell state, tissue type, and/or environment in which the CRE is active in;
delivering occurs in vivo or ex vivo; and
delivery is systemic or local.

113. (canceled)

114. (canceled)

115. (canceled)

116. The method of claim 104, wherein the therapeutic polynucleotide (a) generates one or more genetic or epigenetic mutations, (b) generates a replacement gene product, (c) modulates gene and/or gene product expression, (d) kills or inhibits the growth or infection by a pathogen, (e) modulates one or more cellular activities, functions, or interactions, (f) kills or inhibits cell growth, differentiation, and/or proliferation, or (g) any combination of (a)-(f) in/of the one or more cells in which the therapeutic polynucleotide is expressed.

117. The method of claim 90, wherein the one or more cells comprises or consists of cells selected from: vertebrate cells, invertebrate cells, mammalian cells, avian cells, reptilian cells, fish cells, amphibian cells, insect cells, human cells, non-human primate cells, plant cells, or prokaryotic cells.

118. (canceled)

119. (canceled)

120. (canceled)

121. (canceled)

122. (canceled)

123. (canceled)

124. (canceled)

125. (canceled)
